(19) United States
(12) Patent Application Publication
(10) Pub. No.: US 2004/0067490 A1
(43) Pub. Date: Apr. 8, 2004

(54) Therapeutic polypeptides, nucleic acids encoding same, and methods of use

(76) Inventors: Mei Zhong, Branford, CT (US); Li Li, Branford, CT (US); Linda Gorman, Branford, CT (US); Kimberly A. Spytek, New Haven, CT (US); Ramesh Kekuda, Norwalk, CT (US); Raymond J. Taupier JR., East Haven, CT (US); David W. Anderson, Branford, CT (US); Corine A.M. Vernet, Branford, CT (US); Elina Catterton, Madison, CT (US); Charles E. Miller, Guilford, CT (US); Suresh G. Shenoy, Branford, CT (US); Meera Patturajan, Branford, CT (US); Carol E. A. Pena, New Haven, CT (US); Velizar T. Tchernev, Branford, CT (US); Muralidhara Padigaru, Branford, CT (US); Vladimir Y. Gusev, Madison, CT (US); Uriel M. Malyankar, Branford, CT (US); Catherine E. Burgess, Wethersfield, CT (US); Valerie Gerlach, Branford, CT (US); Stacie J. Casman, North Haven, CT (US); Daniel K. Rieger, Branford, CT (US); William M. Grosse, Branford, CT (US); Glennda Smithson, Guilford, CT (US); John A. Peyman, New Haven, CT (US); Gary Starling, Middletown, CT (US); Mark E. Rothenberg, Clinton, CT (US); William J. LaRochelle, Madison, CT (US); Richard A. Shimkets, Guilford, CT (US); Julie Crabtree, Gainesville, FL (US); Luca Rastelli, Guilford, CT (US); Edward Z. Voss, Wallingford, CT (US); Ferenc L. Boldog, North Haven, CT (US); Shlomit R. Edinger, New Haven, CT (US); Isabelle Millet, Milford, CT (US); John R. MacDougall, Hamden, CT (US); Karen Ellerman, Branford, CT (US); Andrei Chapoval, Branford, CT (US)

Correspondence Address: MINTZ, LEVIN, COHN,, FERRIS, GLOVSKY and POPEO, P.C., One Financial Center, Boston, MA, 02111, US

(21) Appl. No.: 10/236,392

(22) Filed: Sep. 6, 2002

Related U.S. Application Data
(60) Provisional application No. 60/390,155, filed on Jun. 19, 2002.
(60) Provisional application No. 60/318,765, filed on Sep. 12, 2001.
(60) Provisional application No. 60/357,303, filed on Feb. 15, 2002.
(60) Provisional application No. 60/367,753, filed on Mar. 25, 2002.
(60) Provisional application No. 60/369,479, filed on Apr. 2, 2002.
(60) Provisional application No. 60/318,120, filed on Sep. 7, 2001.
(60) Provisional application No. 60/318,130, filed on Sep. 7, 2001.
(60) Provisional application No. 60/381,672, filed on May 17, 2002.
(60) Provisional application No. 60/318,219, filed on Sep. 7, 2001.
(60) Provisional application No. 60/318,430, filed on Sep. 10, 2001.
(60) Provisional application No. 60/322,781, filed on Sep. 17, 2001.
(60) Provisional application No. 60/322,816, filed on Sep. 17, 2001.
(60) Provisional application No. 60/323,519, filed on Sep. 19, 2001.
(60) Provisional application No. 60/384,012, filed on May 29, 2002.
(60) Provisional application No. 60/323,631, filed on Sep. 20, 2001.
(60) Provisional application No. 60/323,636, filed on Sep. 20, 2001.
(60) Provisional application No. 60/360,973, filed on Feb. 28, 2002.
(60) Provisional application No. 60/366,131, filed on Mar. 20, 2002.
(60) Provisional application No. 60/324,969, filed on Sep. 25, 2001.
(60) Provisional application No. 60/383,651, filed on May 28, 2002.
(60) Provisional application No. 60/325,091, filed on Sep. 25, 2001.
(60) Provisional application No. 60/324,990, filed on Sep. 26, 2001.
(60) Provisional application No. 60/381,664, filed on May 17, 2002.
(60) Provisional application No. 60/379,532, filed on May 10, 2002.

(51) Int. Cl. C12Q 1/68; G01N 33/53; A61K 38/17; C12P 21/02; C12N 5/06; C12N 9/00; C07H 21/04

(52) U.S. Cl. 435/6; 435/7.1; 435/69.1; 435/183; 435/320.1; 435/325; 514/12; 530/388.1; 536/23.2

(57) ABSTRACT

Disclosed herein are nucleic acid sequences that encode novel polypeptides. Also disclosed are polypeptides encoded by these nucleic acid sequences, and antibodies that immunospecifically bind to the polypeptide, as well as derivatives, variants, mutants, or fragments of the novel polypeptide, polynucleotide, or antibody specific to the polypeptide. Vectors, host cells, antibodies and recombinant methods for producing the polypeptides and polynucleotides, as well as methods for using same are also included. The invention further discloses therapeutic, diagnostic and research methods for diagnosis, treatment, and prevention of disorders involving any one of these novel human nucleic acids and proteins.

### RELATED APPLICATIONS

This application claims priority to provisional patent applications U.S. Ser. No. 09/540,763 (Cura 43), filed Mar. 30, 2000; U.S. S. No. 60/390,155 (Cura 43 U-A), filed Jun. 19, 2002; U.S. Ser. No. 09/635,949 (Cura 59), filed Aug. 10, 2000; U.S. S. No. 60/318,765 (Cura 59U-A), filed Sep. 12, 2001; U.S. S. No. 60/357,303 (Cura 59U-B), filed Feb. 15, 2002; U.S. S. No. 60/367,753 (Cura 59U-C), filed Mar. 25, 2002; U.S. S. No. 60/369,479 (Cura 59U-D), filed Apr. 2, 2002; U.S. Ser. No. 09/659,634 (Cura 67), filed Sep. 12, 2000; U.S. S. No. 60/318,120 (Cura 742), filed Sep. 7, 2001; U.S. S. No. 60/318,130 (Cura 743), filed Sep. 7, 2001; U.S. S. No. 60/381,672 (Cura 743 JFC-01), filed May 17, 2002; U.S. S. No. 60/318,219 (Cura 744), filed Sep. 7, 2001; U.S. S. No. 60/318,430 (Cura 746), filed Sep. 10, 2001; U.S. S. No. 60/322,781 (Cura 747), filed Sep. 17, 2001; U.S. S. No. 60/322,816 (Cura 751), filed Sep. 17, 2001; U.S. S. No. 60/323,519 (Cura 752), filed Sep. 19, 2001; U.S. S. No. 60/384,012 (Cura 752DI), filed May 29, 2002; U.S. S. No. 60/323,631 (Cura 753), filed Sep. 20, 2001; U.S. S. No. 60/323,636 (Cura 754), filed Sep. 20, 2001; U.S. S. No. 60/360,973 (Cura 754 IFC-01) filed Feb. 28, 2002; U.S. S. No. 60/366,131 (Cura 754G1), filed Mar. 20, 2002; U.S. S. No. 60/324,969 (Cura 755), filed Sep. 25, 2001; U.S. S. No. 60/383,651(Cura 755GJ) filed May 28, 2002; U.S. S. No. 60/325,091(Cura 756), filed Sep. 25, 2001; U.S. S. No. 60/324,990 (Cura 757), filed Sep. 26, 2001; U.S. S. No. 60/381,664 (Cura 757 IFC-01), filed May 17, 2002; U.S. S. No. 60/379,532 (Cura 757H1), filed May 10, 2002, each of which is incorporated herein by reference in its entirety.

### FIELD OF THE INVENTION

The present invention relates to novel polypeptides, and the nucleic acids encoding them, having properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND OF THE INVENTION

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins, and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as

diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Antibodies are multichain proteins that bind specifically to a given antigen, and bind poorly, or not at all, to substances deemed not to be cognate antigens. Antibodies are comprised of two short chains termed light chains and two long chains termed heavy chains. These chains are constituted of immunoglobulin domains, of which generally there are two classes: one variable domain per chain, one constant domain in light chains, and three or more constant domains in heavy chains. The antigen-specific portion of the immunoglobulin molecules resides in the variable domains; the variable domains of one light chain and one heavy chain associate with each other to generate the antigen-binding moiety. Antibodies that bind immunospecifically to a cognate or target antigen bind with high affinities. Accordingly, they are useful in assaying specifically for the presence of the antigen in a sample. In addition, they have the potential of inactivating the activity of the antigen.

Therefore there is a need to assay for the level of a protein effector of interest in a biological sample from such a subject, and to compare this level with that characteristic of a nonpathological condition. In particular, there is a need for such an assay based on the use of an antibody that binds immunospecifically to the antigen. There further is a need to inhibit the activity of the protein effector in cases where a pathological condition arises from elevated or excessive levels of the effector based on the use of an antibody that binds immunospecifically to the effector. Thus, there is a need for the antibody as a product of manufacture. There further is a need for a method of treatment of a pathological condition brought on by an elevated or excessive level of the protein effector of interest based on administering the antibody to the subject.

### SUMMARY OF THE INVENTION

The invention is based in part upon the discovery of isolated polypeptides including amino acid sequences selected from mature forms of the amino acid sequences selected from the group consisting of SEQ ID NO:2n, wherein n is an integer between 1 and 127. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid or polypeptide sequences.

The invention also is based in part upon variants of a mature form of the amino acid sequence selected from the group consisting of SEQ ID NO:2n, wherein n is an integer between 1 and 127, wherein any amino acid in the mature form is changed to a different amino acid, provided that no more than 15% of the amino acid residues in the sequence of the mature form are so changed. In another embodiment, the invention includes the amino acid sequences selected from the group consisting of SEQ ID NO:2n, wherein n is an integer between 1 and 127. In another embodiment, the invention also comprises variants of the amino acid sequence selected from the group consisting of SEQ ID NO:2n, wherein n is an integer between 1 and 127, wherein any amino acid specified in the chosen sequence is changed to a different amino acid, provided that no more than 15% of the amino acid residues in the sequence are so changed. The invention also involves fragments of any of the mature forms of the amino acid sequences selected from the group consisting of SEQ ID NO:2n, wherein n is an integer between 1 and 127, or any other amino acid sequence selected from this group. The invention also comprises fragments from these groups in which up to 15% of the residues are changed.

In another embodiment, the invention encompasses polypeptides that are naturally occurring allelic variants of the sequence selected from the group consisting of SEQ ID NO:2n, wherein n is an integer between 1 and 127. These allelic variants include amino acid sequences that are the translations of nucleic acid sequences differing by a single nucleotide from nucleic acid sequences selected from the group consisting of SEQ ID NOS: 2n−1, wherein n is an integer between 1 and 127. The variant polypeptide where any amino acid changed in the chosen sequence is changed to provide a conservative substitution.

In another embodiment, the invention comprises a pharmaceutical composition involving a polypeptide with an amino acid sequence selected from the group consisting of SEQ ID NO:2n, wherein n is an integer between 1 and 127, and a pharmaceutically acceptable carrier. In another embodiment, the invention involves a kit, including, in one or more containers, this pharmaceutical composition.

In another embodiment, the invention includes the use of a therapeutic in the manufacture of a medicament for treating a syndrome associated with a human disease, the disease being selected from a pathology associated with a polypeptide with an amino acid sequence selected from the group consisting of SEQ ID NO:2n, wherein n is an integer between 1 and 127, wherein said therapeutic is the polypeptide selected from this group.

In another embodiment, the invention comprises a method for determining the presence or amount of a polypeptide with an amino acid sequence selected from the group consisting of SEQ ID NO:2n, wherein n is an integer between 1 and 127, in a sample, the method involving providing the sample; introducing the sample to an antibody that binds immunospecifically to the polypeptide; and determining the presence or amount of antibody bound to the polypeptide, thereby determining the presence or amount of polypeptide in the sample.

In another embodiment, the invention includes a method for determining the presence of or predisposition to a disease associated with altered levels of a polypeptide with an amino acid sequence selected from the group consisting of SEQ ID NO:2n, wherein n is an integer between 1 and 127, in a first mammalian subject, the method involving measuring the level of expression of the polypeptide in a sample from the first mammalian subject; and comparing the amount of the polypeptide in this sample to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In another embodiment, the invention involves a method of identifying an agent that binds to a polypeptide with an amino acid sequence selected from the group consisting of SEQ ID NO:2n, wherein n is an integer between 1 and 127, the method including introducing the polypeptide to the agent; and determining whether the agent binds to the polypeptide. The agent could be a cellular receptor or a downstream effector.

In another embodiment, the invention involves a method for identifying a potential therapeutic agent for use in treatment of a pathology, wherein the pathology is related to aberrant expression or aberrant physiological interactions of a polypeptide with an amino acid sequence selected from the group consisting of SEQ ID NO:2n, wherein n is an integer between 1 and 127, the method including providing a cell expressing the polypeptide of the invention and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent.

In another embodiment, the invention involves a method for screening for a modulator of activity or of latency or predisposition to a pathology associated with a polypeptide having an amino acid sequence selected from the group consisting of SEQ ID NO:2n, wherein n is an integer between 1 and 127, the method including administering a test compound to a test animal at increased risk for a pathology associated with the polypeptide of the invention, wherein the test animal recombinantly expresses the polypeptide of the invention; measuring the activity of the polypeptide in the test animal after administering the test compound; and comparing the activity of the protein in the test animal with the activity of the polypeptide in a control animal not administered the polypeptide, wherein a change in the activity of the polypeptide in the test animal relative to the control animal indicates the test compound is a modulator of latency of, or predisposition to, a pathology associated with the polypeptide of the invention. The recombinant test animal could express a test protein transgene or express the transgene under the control of a promoter at an increased level relative to a wild-type test animal The promoter may or may not b the native gene promoter of the transgene.

In another embodiment, the invention involves a method for modulating the activity of a polypeptide with an amino acid sequence selected from the group consisting of SEQ ID NO:2n, wherein n is an integer between 1 and 127, the method including introducing a cell sample expressing the polypeptide with a compound that binds to the polypeptide in an amount sufficient to modulate the activity of the polypeptide.

In another embodiment, the invention involves a method of treating or preventing a pathology associated with a polypeptide with an amino acid sequence selected from the group consisting of SEQ ID NO:2n, wherein n is an integer between 1 and 127, the method including administering the polypeptide to a subject in which such treatment or prevention is desired in an amount sufficient to treat or prevent the pathology in the subject. The subject could be human.

In another embodiment, the invention involves a method of treating a pathological state in a mammal, the method including administering to the mammal a polypeptide in an amount that is sufficient to alleviate the pathological state, wherein the polypeptide is a polypeptide having an amino acid sequence at least 95% identical to a polypeptide having the amino acid sequence selected from the group consisting of SEQ ID NO:2n, wherein n is an integer between 1 and 127, or a biologically active fragment thereof.

In another embodiment, the invention involves an isolated nucleic acid molecule comprising a nucleic acid sequence encoding a polypeptide having an amino acid sequence selected from the group consisting of a mature form of the amino acid sequence given SEQ ID NO:2n, wherein n is an integer between 1 and 127; a variant of a mature form of the amino acid sequence selected from the group consisting of SEQ ID NO:2n, wherein n is an integer between 1 and 127, wherein any amino acid in the mature form of the chosen sequence is changed to a different amino acid, provided that no more than 15% of the amino acid residues in the sequence of the mature form are so changed; the amino acid sequence selected from the group consisting of SEQ ID NO:2n, wherein n is an integer between 1 and 127; a variant of the amino acid sequence selected from the group consisting of SEQ ID NO:2n, wherein n is an integer between 1 and 127, in which any amino acid specified in the chosen sequence is changed to a different amino acid, provided that no more than 15% of the amino acid residues in the sequence are so changed; a nucleic acid, fragment encoding at least a portion of a polypeptide comprising the amino acid sequence selected from the group consisting of SEQ ID NO:2n, wherein n is an integer between 1 and 127, or any variant of the polypeptide wherein any amino acid of the chosen sequence is changed to a different amino acid, provided that no more than 10% of the amino acid residues in the sequence are so changed; and the complement of any of the nucleic acid molecules.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 127, wherein the nucleic acid molecule comprises the nucleotide sequence of a naturally occurring allelic nucleic acid variant.

In another embodiment, the invention involves an isolated nucleic acid molecule including a nucleic acid sequence encoding a polypeptide having an amino acid sequence selected from the group consisting of a mature form of the amino acid sequence given SEQ ID NO:2n, wherein n is an integer between 1 and 127, that encodes a variant polypeptide, wherein the variant polypeptide has the polypeptide sequence of a naturally occurring polypeptide variant.

In another embodiment, the invention comprises an isolated nucleic acid molecule having a nucleic acid sequence encoding a polypeptide comprising an amino acid sequence selected from the group consisting of a mature form of the amino acid sequence given SEQ ID NO:2n, wherein n is an integer between 1 and 127, wherein the nucleic acid molecule differs by a single nucleotide from a nucleic acid sequence selected from the group consisting of SEQ ID NOS: 2n−1, wherein n is an integer between 1 and 127.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 127, wherein the nucleic acid molecule comprises a nucleotide sequence selected from the group consisting of the nucleotide sequence selected from the group consisting of SEQ ID NO:2n−1, wherein n is an integer between 1 and 127; a nucleotide sequence wherein one or more nucleotides in the nucleotide sequence selected from the group consisting of SEQ ID NO:2n−1, wherein n is an integer between 1 and 127, is changed from that selected from the group consisting of the chosen sequence to a different nucleotide provided that no more than 15% of the nucleotides are so changed; a nucleic acid fragment of the sequence selected from the group consisting of SEQ ID NO:2n−1, wherein n is an integer between 1 and 127; and a nucleic acid fragment wherein one or more nucleotides in the nucleotide sequence selected from the group consisting of SEQ ID NO:2n−1, wherein n is an integer between 1 and 127, is changed from that selected from the group consisting of the chosen sequence to a different nucleotide provided that no more than 15% of the nucleotides are so changed.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 127, wherein the nucleic acid molecule hybridizes under stringent conditions to the nucleotide sequence selected from the group consisting of SEQ ID NO:2n−1, wherein n is an integer between 1 and 127, or a complement of the nucleotide sequence.

In another embodiment, the invention includes an isolated nucleic acid molecule having a nucleic acid sequence encoding a polypeptide including an amino acid sequence selected from the group consisting of a mature form of the amino acid sequence given SEQ ID NO:2n, wherein n is an integer between 1 and 127, wherein the nucleic acid molecule has a nucleotide sequence in which any nucleotide specified in the coding sequence of the chosen nucleotide sequence is changed from that selected from the group consisting of the chosen sequence to a different nucleotide provided that no more than 15% of the nucleotides in the chosen coding sequence are so changed, an isolated second polynucleotide that is a complement of the first polynucleotide, or a fragment of any of them.

In another embodiment, the invention includes a vector involving the nucleic acid molecule having a nucleic acid sequence encoding a polypeptide including an amino acid sequence selected from the group consisting of a mature form of the amino acid sequence given SEQ ID NO:2n, wherein n is an integer between 1 and 127. This vector can have a promoter operably linked to the nucleic acid molecule This vector can be located within a cell.

In another embodiment, the invention involves a method for determining the presence or amount of a nucleic acid molecule having a nucleic acid sequence encoding a polypeptide including an amino acid sequence selected from the group consisting of a mature form of the amino acid sequence given SEQ ID NO:2n, wherein n is an integer between 1 and 127, in a sample, the method including providing the sample; introducing the sample to a probe that binds to the nucleic acid molecule; and determining the presence or amount of the probe bound to the nucleic acid molecule, thereby determining the presence or amount of the nucleic acid molecule in the sample. The presence or amount of the nucleic acid molecule is used as a marker for cell or tissue type. The cell type can be cancerous.

In another embodiment, the invention involves a method for determining the presence of or predisposition for a disease associated with altered levels of a nucleic acid molecule having a nucleic acid sequence encoding a polypeptide including an amino acid sequence selected from the group consisting of a mature form of the amino acid sequence given SEQ ID NO:2n, wherein n is an integer between 1 and 127, in a first mammalian subject, the method including measuring the amount of the nucleic acid in a sample from the first mammalian subject; and comparing the amount of the nucleic acid in the sample of step (a) to the amount of the nucleic acid present in a control sample from a second mammalian subject known not to have or not be predisposed to, the disease; wherein an alteration in the level of the nucleic acid in the first subject as compared to the control sample indicates the presence of or predisposition to the disease.

The invention further provides an antibody that binds immunospecifically to a NOVX polypeptide. The NOVX antibody may be monoclonal, humanized, or a fully human antibody. Preferably, the antibody has a dissociation constant for the binding of the NOVX polypeptide to the antibody less than 1×10⁻⁹ M. More preferably, the NOVX antibody neutralizes the activity of the NOVX polypeptide.

In a further aspect, the invention provides for the use of a therapeutic in the manufacture of a medicament for treating a syndrome associated with a human disease, associated with a NOVX polypeptide. Preferably the therapeutic is a NOVX antibody.

In yet a further aspect, the invention provides a method of treating or preventing a NOVX-associated disorder, a method of treating a pathological state in a mammal, and a method of treating or preventing a pathology associated with a polypeptide by administering a NOVX antibody to a subject in an amount sufficient to treat or prevent the disorder.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and are not intended to be limiting.

Other features and advantages of the invention will be apparent from the following detailed description and claims.

### BRIEF DESCRIPTION OF THE FIGURES

FIG. 1 is a Western blot showing expression of NOV30b (CG51117-05) immunoreactive polypeptide in human embryonic kidney 293 cells.

FIG. 2 is a schematic diagram of the x-ray crystal structure of porcine colipase and tetra ethylene glycol monooctyl ether inhibitor.

FIG. 3 is a schematic diagram showing the interfacial binding domain of colipase.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| NOVX | Internal | NO | NO | |
| Assignment | Identification | (nucleic acid) | (amino acid) | Homology |
| NOV1a | CG108440- | 1 | 2 | Fibronectin precursor protein-like |
| | 01 | | | protein |
| NOV1b | CG108440- | 3 | 4 | Fibronectin precursor protein-like |
| | 02 | | | protein |
| NOV2a | CG122589- | 5 | 6 | Asialoglycoprotein receptor 2-like |
| | 01 | | | protein |
| NOV2b | CG122589- | 7 | 8 | Asialoglycoprotein receptor 2-like |
| | 02 | | | protein |
| NOV2c | CG122589- | 9 | 10 | Asialoglycoprotein receptor 2-like |
| | 03 | | | protein |
| NOV3a | CG133274- | 11 | 12 | Induced myeloid leukemia cell |
| | 01 | | | differentiation protein MCL-1-like |
| | | | | protein |
| NOV3b | CG133274- | 13 | 14 | Induced myeloid leukemia cell |
| | 02 | | | differentiation protein MCL-1-like |
| | | | | protein |
| NOV3c | 278876765 | 15 | 16 | Induced myeloid leukemia cell |
| | | | | differentiation protein MCL-1-like |
| | | | | protein |
| NOV3d | 278881214 | 17 | 18 | Induced myeloid leukemia cell |
| | | | | differentiation protein MCL-1-like |
| | | | | protein |
| NOV4a | CG134430- | 19 | 20 | RIKEN cDNA 2310034104-like |
| | 01 | | | protein |
| NOV5a | CG137677- | 21 | 22 | RIKEN 5730409G15-like protein |
| | 01 | | | |
| NOV6a | CG137697- | 23 | 24 | RIKEN 5730409G15-like protein |
| | 01 | | | |
| NOV7a | CG137717- | 25 | 26 | FLJ37712 fis protein-like protein |
| | 01 | | | |
| NOV8a | CG137793- | 27 | 28 | High affinity immunoglobulin |
| | 01 | | | epsilon receptor alpha subunit |
| | | | | precursor protein-like protein |
| NOV8b | CG137793- | 29 | 30 | High affinity immunoglobulin |
| | 02 | | | epsilon receptor alpha subunit |
| | | | | precursor protein-like protein |
| NOV9a | CG137873- | 31 | 32 | Fibrinogen alpha chain precursor |
| | 01 | | | protein-like protein |
| NOV9b | CG137873- | 33 | 34 | Fibrinogen alpha chain precursor |
| | 03 | | | protein-like protein |
| NOV9c | CG137873- | 35 | 36 | Fibrinogen alpha chain precursor |
| | 02 | | | protein-like protein |
| NOV10a | CG137882- | 37 | 38 | FLJ21269-like protein |
| | 01 | | | |
| NOV10b | CG137882- | 39 | 40 | FLJ21269-like protein |
| | 02 | | | |
| NOV11a | CG137910- | 41 | 42 | FLJ21432-like protein |
| | 01 | | | |
| NOV12a | CG138013- | 43 | 44 | Sialic acid-binding |
| | 01 | | | immunoglobulin-like lectin-9-like |
| | | | | protein |
| NOV13a | CG138074- | 45 | 46 | RIKEN 2310012P03-like protein |
| | 01 | | | |
| NOV14a | CG138573- | 47 | 48 | Folate receptor 3-like protein |
| | 01 | | | |
| NOV15a | CG138606- | 49 | 50 | Brush border 61.9 kDa protein |
| | 01 | | | precursor-like protein |
| NOV16a | CG138751- | 51 | 52 | cAMP inducible 2 protein-like |
| | 01 | | | protein |
| NOV16b | CG138751- | 53 | 54 | cAMP inducible 2 protein-like |
| | 02 | | | protein |
| NOV17a | CG139062- | 55 | 56 | Jagged 1 precursor protein-like |
| | 01 | | | protein |
| NOV17b | CG139062- | 57 | 58 | Jagged 1 precursor protein-like |
| | 02 | | | protein |
| NOV18a | CG139363- | 59 | 60 | Transmembrane protein HTMP10- |
| | 01 | | | like protein |
| NOV18b | CG139363- | 61 | 62 | Transmembrane protein HTMP10- |
| | 02 | | | like protein |
| NOV19a | CG140188- | 63 | 64 | DC2-like protein |
| | 01 | | | |
| NOV20a | CG140305- | 65 | 66 | Complement-c1q tumor necrosis |
| | 01 | | | factor-related protein-like protein |
| NOV20b | CG140305- | 67 | 68 | Complement-c1q tumor necrosis |
| | 02 | | | factor-related protein-like protein |
| NOV21a | CG140639- | 69 | 70 | Flotillin-2 (Reggie-1) (REG-1)- |
| | 01 | | | like protein |
| NOV21b | CG140639- | 71 | 72 | Flotillin-2 (Reggie-1) (REG-1)- |
| | 02 | | | like protein |
| NOV22a | CG140843- | 73 | 74 | Integrin beta-5 precursor protein- |
| | 01 | | | like protein |
| NOV23a | CG141540- | 75 | 76 | IL1 receptor-type 2-like protein |
| | 01 | | | |
| NOV23b | CG141540- | 77 | 78 | IL1 receptor-type 2-like protein |
| | 02 | | | |
| NOV24a | CG141580- | 79 | 80 | KIAA 1467 protein-like protein |
| | 01 | | | |
| NOV25a | CG141643- | 81 | 82 | RIKEN 2010001CC9 protein-like |
| | 01 | | | protein |
| NOV26a | CG142003- | 83 | 84 | Plasma protease C1 inhibitor |
| | 01 | | | precursor protein-like protein |
| NOV26b | 306076006 | 85 | 86 | Plasma protease C1 inhibitor |
| | | | | precursor protein-like protein |
| NOV26c | 278889088 | 87 | 88 | Plasma protease C1 inhibitor |
| | | | | precursor protein-like protein |
| NOV26d | CG142003- | 89 | 90 | Plasma protease C1 inhibitor |
| | 02 | | | precursor protein-like protein |
| NOV27a | CG142023- | 91 | 92 | 6230421J19Rik protein-like protein |
| | 01 | | | |
| NOV28a | CG142092- | 93 | 94 | C4b-binding protein alpha chain |
| | 01 | | | precursor protein-like protein |
| NOV28b | CG142092- | 95 | 96 | C4b-binding protein alpha chain |
| | 02 | | | precursor protein-like protein |
| NOV28c | CG142092- | 97 | 98 | C4b-binding protein alpha chain |
| | 03 | | | precursor protein-like protein |
| NOV29a | CG171681- | 99 | 100 | Sushi repeat-containing protein |
| | 01 | | | |
| NOV29b | CG171681- | 101 | 102 | Sushi repeat-containing protein |
| | 03 | | | |
| NOV29c | CG171681- | 103 | 104 | Sushi repeat-containing protein |
| | 02 | | | |
| NOV30a | CG51117-01 | 105 | 106 | Nephronectin-like protein |
| NOV30b | CG51117-05 | 107 | 108 | Nephronectin-like protein |
| NOV30c | CG51117-06 | 109 | 110 | Nephronectin-like protein |
| NOV30d | CG51117-07 | 111 | 112 | Nephronectin-like protein |
| NOV30e | CG51117-03 | 113 | 114 | Nephronectin-like protein |
| NOV30f | CG51117-02 | 115 | 116 | Nephronectin-like protein |
| NOV30g | CG51117-04 | 117 | 118 | Nephronectin-like protein |
| NOV30h | CG51117-08 | 119 | 120 | Nephronectin-like protein |
| NOV30i | CG51117-09 | 121 | 122 | Nephronectin-like protein |
| NOV31a | CG51264-01 | 123 | 124 | ST7-like protein |
| NOV31b | CG51264-03 | 125 | 126 | ST7-like protein |
| NOV31c | CG51264-04 | 127 | 128 | ST7-like protein |
| NOV31d | CG51264-06 | 129 | 130 | ST7-like protein |
| NOV31e | CG51264-07 | 131 | 132 | ST7-like protein |
| NOV31f | CG51264-02 | 133 | 134 | ST7-like protein |
| NOV31g | CG51264-05 | 135 | 136 | ST7-like protein |
| NOV31h | CG51264-08 | 137 | 138 | ST7-like protein |
| NOV31i | CG51264-09 | 139 | 140 | ST7-like protein |
| NOV31j | CG51264-10 | 141 | 142 | ST7-like protein |
| NOV31k | CG51264-11 | 143 | 144 | ST7-like protein |
| NOV31l | CG51264-12 | 145 | 146 | ST7-like protein |
| NOV31m | CG51264-13 | 147 | 148 | ST7-like protein |
| NOV31n | CG51264-14 | 149 | 150 | ST7-like protein |
| NOV31o | CG51264-15 | 151 | 152 | ST7-like protein |
| NOV31p | CG51264-16 | 153 | 154 | ST7-like protein |
| NOV32a | CG52423-01 | 155 | 156 | PV-1-like protein |
| NOV33a | CG52919-01 | 157 | 158 | Sez-6-like protein |
| NOV33b | CG52919-02 | 159 | 160 | Sez-6-like protein |
| NOV33c | CG52919-03 | 161 | 162 | Sez-6-like protein |
| NOV33d | CG52919-04 | 163 | 164 | Sez-6-like protein |
| NOV33e | CG52919-05 | 165 | 166 | Sez-6-like protein |
| NOV33f | CG52919-06 | 167 | 168 | Sez-6-like protein |
| NOV33g | CG52919-01 | 169 | 170 | Sez-6-like protein |
| NOV33h | CG52919-07 | 171 | 172 | Sez-6-like protein |
| NOV33i | CG52919-08 | 173 | 174 | Sez-6-like protein |
| NOV33j | CG52919-09 | 175 | 176 | Sez-6-like protein |
| NOV34a | CG55698-01 | 177 | 178 | Colipase precursor protein-like |
| | | | | protein |
| NOV34b | CG55698-02 | 179 | 180 | Colipase precursor protein-like |
| | | | | protein |
| NOV35a | CG55832-01 | 181 | 182 | Tenascin-C precursor protein-like |
| | | | | protein |
| NOV35b | CG55832-03 | 183 | 184 | Tenascin-C precursor protein-like |
| | | | | protein |
| NOV35c | CG55832-02 | 185 | 186 | Tenascin-C precursor protein-like |
| | | | | protein |
| NOV36a | CG56054-01 | 187 | 188 | Integrin alpha 7-like protein |
| NOV36b | CG56054-03 | 189 | 190 | Integrin alpha 7-like protein |
| NOV36c | CG56054-04 | 191 | 192 | Integrin alpha 7-like protein |
| NOV36d | CG56054-05 | 193 | 194 | Integrin alpha 7-like protein |
| NOV36e | CG56054-06 | 195 | 196 | Integrin alpha 7-like protein |
| NOV36f | CG56054-07 | 197 | 198 | Integrin alpha 7-like protein |
| NOV36g | CG56054-08 | 199 | 200 | Integrin alpha 7-like protein |
| NOV36h | CG56054-09 | 201 | 202 | Integrin alpha 7-like protein |
| NOV36i | CG56054-10 | 203 | 204 | Integrin alpha 7-like protein |
| NOV36j | CG56054-11 | 205 | 206 | Integrin alpha 7-like protein |
| NOV36k | CG56054-12 | 207 | 208 | Integrin alpha 7-like protein |
| NOV36l | CG56054-13 | 209 | 210 | Integrin alpha 7-like protein |
| NOV36m | CG56054-14 | 211 | 212 | Integrin alpha 7-like protein |
| NOV36n | CG56054-15 | 213 | 214 | Integrin alpha 7-like protein |
| NOV36o | CG56054-16 | 215 | 216 | Integrin alpha 7-like protein |
| NOV36p | CG56054-17 | 217 | 218 | Integrin alpha 7-like protein |
| NOV36q | CG56054-18 | 219 | 220 | Integrin alpha 7-like protein |
| NOV36r | CG56054-19 | 221 | 222 | Integrin alpha 7-like protein |
| NOV36s | CG56054-02 | 223 | 224 | Integrin alpha 7-like protein |
| NOV37a | CG88634-01 | 225 | 226 | KIAA1219-like protein |
| NOV38a | CG97012-01 | 227 | 228 | Seizure 6 precursor protein-like |
| | | | | protein |
| NOV38b | CG97012-02 | 229 | 230 | Seizure 6 precursor protein-like |
| | | | | protein |
| NOV38c | CG97012-03 | 231 | 232 | Seizure 6 precursor protein-like |
| | | | | protein |
| NOV38d | CG97012-01 | 233 | 234 | Seizure 6 precursor protein-like |
| | | | | protein |
| NOV38e | 210120300 | 235 | 236 | Seizure 6 precursor protein-like |
| | | | | protein |
| NOV38f | 210120376 | 237 | 238 | Seizure 6 precursor protein-like |
| | | | | protein |
| NOV38g | 210120463 | 239 | 240 | Seizure 6 precursor protein-like |
| | | | | protein |
| NOV38h | 210120269 | 241 | 242 | Seizure 6 precursor protein-like |
| | | | | protein |
| NOV38i | CG97012-04 | 243 | 244 | Seizure 6 precursor protein-like |
| | | | | protein |
| NOV38j | CG97012-05 | 245 | 246 | Seizure 6 precursor protein-like |
| | | | | protein |
| NOV39a | CG99754-01 | 247 | 248 | RIKEN protein-like protein |
| NOV39b | CG99754-02 | 249 | 250 | RIKEN protein-like protein |
| NOV40a | CG99777-01 | 251 | 252 | CD30 ligand-like protein |
| NOV40b | CG99777-02 | 253 | 254 | CD30 ligand-like protein |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders, conditions, and the like that are associated with NOVX sequences include, but are not limited to: e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, adrenoleukodystrophy, congenital adrenal hyperplasia, prostate cancer, diabetes, metabolic disorders, neoplasm, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, neurodegenerative disorders, Alzheimer's Disease, Parkinson's Disease, immune disorders, hematopoietic disorders, and the various dyslipidemias, the metabolic syndrome X, wasting disorders associated with chronic diseases, cancer, e.g., uterine cancer, lymphoma, adenocarcinoma, as well as conditions such as transplantation, neuroprotection, fertility, or regeneration (in vitro and in vivo).

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 127; (b) a variant of a mature form of the amino acid sequence selected from the group consisting of SEQ ID NO: 2n, wherein n is an integer between 1 and 127, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 127; (d) a variant of the amino acid sequence selected from the group consisting of SEQ ID NO:2n, wherein n is an integer between 1 and 127, wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 127; (b) a variant of a mature form of the amino acid sequence selected from the group consisting of SEQ ID NO: 2n, wherein n is an integer between 1 and 127, wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 127; (d) a variant of the amino acid sequence selected from the group consisting of SEQ ID NO: 2n, wherein n is an integer between 1 and 127, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 127, or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 127; (b) a nucleotide sequence wherein one or more nucleotides in the nucleotide sequence selected from the group consisting of SEQ ID NO: 2n−1, wherein n is an integer between 1 and 127 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 127; and (d) a nucleic acid fragment wherein one or more nucleotides in the nucleotide sequence selected from the group consisting of SEQ ID NO: 2n−1, wherein It is an integer between 1 and 127, is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues I to N, where residue I is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb, or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 127, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 127, as a hybridization probe, NOVX molecules can be isolated using standard hybridization and cloning techniques (e.g., as described in Sambrook, et al., (eds.), MOLECULAR CLONING: A LABORATORY MANUAL 2,d Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 127, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 127, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 127, is one that is sufficiently complementary to the nucleotide sequence of SEQ ID NO:2n−1, wherein n is an integer between 1 and 127, that it can hydrogen bond with few or no mismatches to the nucleotide sequence shown in SEQ ID NO:2n−1, wherein n is an integer between 1 and 127, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 127, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologs in other cell types, e.g. from other tissues, as well as NOVX homologs from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 127; or an anti-sense strand nucleotide sequence of SEQ ID NO:2n−1, wherein n is an integer between 1 and 127; or of a naturally occurring mutant of SEQ ID NO:2n−1, wherein n is an integer between 1 and 127.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 127, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 127, due to degeneracy of the genetic code and thus encode the same NOVX proteins as that encoded by the nucleotide sequences of SEQ ID NO:2n−1, wherein n is an integer between 1 and 127. In another embodiment, an isolated nucleic acid molecule of the invention has a nucleotide sequence encoding a protein having an amino acid sequence of SEQ ID NO:2n, wherein n is an integer between 1 and 127.

In addition to the human NOVX nucleotide sequences of SEQ ID NO:2n−1, wherein n is an integer between 1 and 127, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 127, are intended to be within the scope of the invention. Nucleic acid molecules corresponding to natural allelic variants and homologs of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 127. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the tern “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 127, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 127, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that maybe used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 127, or fragments, analogs or derivatives thereof under conditions of low stringency, is provided. A non-limiting example of low stringency hybridization conditions are hybridization in 35% formamide, 5×SSC, 50 mM Tris-HCl (p117.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 127, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 127. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 127, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 127. Preferably, the protein encoded by the nucleic acid molecule is at least about 60% homologous to SEQ ID NO:2n, wherein l is an integer between 1 and 127; more preferably at least about 70% homologous to SEQ ID NO:2n, wherein ii is an integer between 1 and 127; still more preferably at least about 80% homologous to SEQ ID NO:2n, wherein n is an integer between 1 and 127; even more preferably at least about 90% homologous to SEQ ID NO:2n, wherein n is an integer between 1 and 127; and most preferably at least about 95% homologous to SEQ ID NO:2n, wherein n is an integer between 1 and 127.

An isolated nucleic acid molecule encoding a NOVX protein homologous to the protein of SEQ ID NO:2n, wherein n is an integer between 1 and 127, can be created by introducing one or more nucleotide substitutions, additions or deletions into the nucleotide sequence of SEQ ID NO:2n−1, wherein n is an integer between 1 and 127, such that one or more amino acid substitutions, additions or deletions are introduced into the encoded protein.

Mutations can be introduced any one of SEQ ID NO:2n−1, wherein n is an integer between 1 and 127, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 127, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, Such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-it antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H 1. One example of a vector system is the GeneSuppressor RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stein-loop transcript.

A siRNA vector appears to have an advantage over synthetic siRNAs where long tern knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N 19) residues (e.g., AA(N 19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target in RNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX⁻) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al. Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2× proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×10⁵ cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO:2n−1, wherein n is an integer between 1 and 127, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 127, or antisense nucleic acids complementary to a NOVX nucleic acid sequence of SEQ ID NO:2n−1, wherein n is an integer between 1 and 127, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5⁹′-methoxycarboxymethyluracil, 2-methylthio-N-6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an a-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 127). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996, supra; Perry-O'Keefe, et al., 1996, Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996, supra); or as probes or primers for DNA sequence and hybridization (See, Hyrup, et al, 1996, supra; Perry-O'Keefe, et al., 1996, supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996, supra). The synthesis of PNA-DNA chimeras can be performed as described in Hyrup, et al., 1996, supra and Finn, et al., 1996, Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, el al, 1996, supra. Alternatively, chimeric molecules can be synthesized with a 5′ DNA segment and a 3′ PNA segment. See, e.g., Petersen, et al, 1975. Bioorg. Med. Chem. Lett. 5: 1119-11124.

In other embodiments, the oligonucleotide may include other appended groups such as peptides (e.g., for targeting host cell receptors in viva), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 127. The invention also includes a mutant or variant protein any of whose residues may be changed from the corresponding residues shown in any one of SEQ ID NO:2n, wherein n is an integer between 1 and 127, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 127) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 127. In other embodiments, the NOVX protein is substantially homologous to SEQ ID NO:2n, wherein n is an integer between 1 and 127, and retains the functional activity of the protein of SEQ ID NO:2n, wherein n is an integer between 1 and 127, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 127, and retains the functional activity of the NOVX proteins of SEQ ID NO:2n, wherein n is an integer between 1 and 127.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package fee, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 127.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 127, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Youvan, 1992, Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The tern “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 127, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, el al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of Fab expression libraries (see e.g., Huse, et al., 1989 Science 246: 1275-1281) to allow rapid and effective identification of monoclonal Fab fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published May 13, 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fe region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fe regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or Immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-in microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscatawvay, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier etc l., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Nucl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1 and 127, can be introduced as a transgene into the genome of a non-human animal. Alternatively, a non-human homolog of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 127), but more preferably, is a non-human homolog of a human NOVX gene. For example, a mouse homolog of human NOVX gene of SEQ ID NO:2n−1, wherein n is an integer between 1 and 127, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then Cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e g, inhalation), transdermal (ie., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X, as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl-N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14:920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 127, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 127, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 127, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in silt hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al., 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996, Human Mutation 7: 244-255; Kozal, et al., 1996, Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996, Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, el al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996, Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof, or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | SEQ ID NO: 1 | 6988 bp | |
|---|---|---|---|
| NOV1a,CG108 | GAAGAGCAAGAGGCAGGCTCAGCAAATGGTTCAGCCCCAGTCCCCGGTGGCTGTCAGTCAA | | |
| 44O-01 DNA | | | |
| Sequence | AGCAAGCCCGGTTGTTATGACAATGGAAAACACTATCAGATAAATCAACAGTGGGAGCGGA | | |
| | CCTACCTAGGTAATGTGTTGGTTTGTACTTGTTATGGAGGAAGCCGAGGTTTTAACTGCGA | | |
| | AAGTAAACCTGAAGCTGAAGAGACTTGCTTTGACAAGTACACTGGGAACACTTACCGAGTG | | |
| | GGTGACACTTATGAGCGTCCTAAAGACTCCATGATCTGGGACTGTACCTGCATCGGGGCTG | | |
| | GGCGAGGGAGAATAAGCTGTACCATCGCAAACCGCTGCCATGAAGGGGGTCAGTCCTACAA | | |
| | GATTGGTGACACCTGGAGCAGACCACATGAGACTGGTGGTTACATGTTAGAGTGTGTCTGT | | |
| | CTTGGTAATGGAAAAGGAGAATGGACCTGCAAGCCCATAGCTGAGAAGTGTTTTGATCATG | | |
| | CTGCTGGGACTTCCTATGTGGTCGGAGAAACGTGGGAGAAGCCCTACCAAGGCTGGATGAT | | |
| | GGTAGATTGTACTTGCCTGGGAGAAGGCAGCGGACGCATCACTTGCACTTCTAGAAATAGA | | |
| | TGCAACGATCAGGACACAAGGACATCCTATAGAATTGGAGACACCTGGAGCAAGAAGGATA | | |
| | ATCGAGGAAACCTGCTCCAGTGCATCTGCACAGGCAACGGCCGAGGAGAGTGGAAGTGTGA | | |
| | GAGGCACACCTCTGTGCAGACCACATCGAGCGGATCTGGCCCCTTCACCGATGTTCGTGCA | | |
| | GCTGTTTACCAACCGCAGCCTCACCCCCAGCCTCCTCCCTATGGCCACTGTGTCACAGACA | | |
| | GTGGTGTGGTCTACTCTGTGGGGATGCAGTGGTTGAAGACACAAGGAAATAAGCAAATGCT | | |
| | TTGCACGTCCCTGGGCAACGGAGTCACCTGCCAAGAGACAGCTGTAACCCAGACTTACGGT | | |
| | GGCAACTTAAATGGAGAGCCATGTGTCTTACCATTCACCTACAATGGCAGGACGTTCTACT | | |
| | CCTGCACCACGGAAGCGCGACACGACGGACATCTTTGGTGCAGCACAACTTCGAATTATGA | | |
| | GCAGGACCAGAAATACTCTTTCTGCACAGACCACACTGTTTTGGTTCAGACTCAAGCAGGA | | |
| | AATTCCAATGGTGCCTTGTGCCACTTCCCCTTCCTATACAACAACCACAATTACACTGATT | | |
| | GCACTTCTGAGGGCAGAAGAGACAACATGAAGTGGTGTGGGACCACACAGAACTATGATGC | | |
| | CGACCAGAAGTTTGGGTTCTGCCCCATCGCTGCCCACGAGGAAATCTGCACAACCAATGAA | | |
| | GGGGTCATGTACCGCATTGGAGATCACTCGCATAAGCACCATGACATGGGTCACATGATGA | | |
| | GGTGCACGTGTGTTGGCAATGGTCGTGGGGAATGGACATGCATTGCCTACTCGCAACTTCC | | |
| | AGATCAGTGCATTGTTGATGACATCACTTACAATGTGAACGACACATTCCACAAGCGTCAT | | |
| | GAAGAGCGGCACATGCTGAACTGTACATGCTTCGGTCAGGGTCGGGGCAGGTGGAAGTGTG | | |
| | ATCCCGTCGACCAATGCCAGGATTCAGAGACTGGGACGTTTTATCAAATTGGAGATTCATG | | |
| | GGAGAAGTATGTGCATGGTGTCAGATACCAGTGCTACTGCTATGGCCGTGGCATTGGGGAG | | |
| | TGGCATTGCCAACCTTTACAGACCTATCCAAGCTCAAGTGGTCCTGTCGAAGTATTTATCA | | |
| | CTGAGACTCCGAGTCAGCCCAACTCCCACCCCATCCAGTGGAATGCACCACAGCCATCTCA | | |
| | CATTTCCAAGTACATTCTCAGGTGGAGACCTAAAAATTCTGTAGGCCGTTGGAAGGAAGCT | | |
| | ACCATACCAGGCCACTTAAACTCCTACACCATCAAAGGCCTCAACCCTGGTGTGGTATACG | | |
| | AGGGCCAGCTCATCAGCATCCAGCAGTACGGCCACCAAGAAGTCACTCGCTTTGACTTCAC | | |
| | CACCACCAGCACCAGCACACCTCTGACCAGCAACACCGTGACAGGAGAGACGACTCCCTTT | | |
| | TCTCCTCTTGTGGCCACTTCTGAATCTGTGACCGAAATCACAGCCAGTAGCTTTGTGGTCT | | |
| | CCTGGGTCTCAGCTTCCGACACCGTGTCGGGATTCCGGGTGGAATATGAGCTGAGTGAGGA | | |
| | GGGAGATGAGCCACAGTACCTGGATCTTCCAAGCACAGCCACTTCTGTGAACATCCCTGAC | | |
| | CTGCTTCCTGGCCGAAAATACATTGTAAATGTCTATCAGATATCTGAGGATGGGGAGCAGA | | |
| | GTTTGATCCTGTCTACTTCACAAACAACAGCGCCTGATGCCCCTCCTGACCCGACTGTGGA | | |
| | CCAAGTTCATGACACCTCAATTGTTCTTCGCTGGAGCAGACCCCAGGCTCCCATCACAGGG | | |
| | TACAGAATAGTCTATTCGCCATCAGTAGAAGGTAGCAGCACAGAACTCAACCTTCCTGAAA | | |
| | CTGCAAACTCCGTCACCCTCAGTGACTTGCAACCTGGTGTTCAGTATAACATCACTATCTA | | |
| | TGCTGTGGAAGAAAATCAACAAAGTACACCTGTTGTCATTCAACAACAAACCACTGGCACC | | |
| | CCACGCTCAGATACAGTGCCCTCTCCCAGGCACCTGCAGTTTGTGGAAGTGACAGACGTGA | | |
| | AGGTCACCATCATGTGGACACCGCCTGAGAGTGCAGTGACCGGCTACCGTGTGGATGTGAT | | |
| | CCCCGTCAACCTCCCTGGCGAGCACCGGCAGAGGCTGCCCATCAGCAGGAACACCTTTGCA | | |
| | GAAGTCACCGGGCTGTCCCCTGGGGTCACCTATTACTTCAAAGTCTTTGCAGTGAGCCATG | | |
| | GGAGGGAGAGCAAGCCTCTGACTGCTCAACAGACAACCAAACTGGATGCTCCCACTAACCT | | |
| | CCAGTTTGTCAATGAAACTGATTCTACTGTCCTGGTGAGATGGACTCCACCTCGGCCCCAG | | |
| | ATAACAGGATACCGACTGACCGTGGGCCTTACCCGAAGAGGCCAGCCCAGGCAGTACAATG | | |
| | TGGGTCCCTCTGTCTCCAAGTACCCCCTGAGGAATCTGCAGCCTGCATCTGAGTACACCGT | | |
| | ATCCCTCGTGGCCATAAAGGGCAACCAAGAGAGCCCCAAAGCCACTGGAGTCTTTACCACA | | |
| | CTGCAGCCTGGGAGCTCTATTCCACCTTACAACACCGAGGTGACTGAGACCACCATCCTGA | | |
| | TCACATGGACGCCTGCTCCAACAATTGGTTTTAAGCTGGGTGTACGACCAAGCCAGGGAGG | | |
| | AGAGGCACCACGAGAAGTGACTTCAGACTCAGGAAGCATCGTTGTGTCCGGCTTGACTCCA | | |
| | GGAGTAGAATACGTCTACACCATCCAAGTCCTGAGAGATGGACAGGAAAGAGATGCGCCAA | | |
| | TTGTAAACAAAGTGGTGACACCATTGTCTCCACCAACAAACTTGCATCTGGAGGCAAACCC | | |
| | TGACACTGGAGTGCTCACAGTCTCCTGGGAGAGGAGCACCACCCCAGACATTACTGGTTAT | | |
| | AGAATTACCACAACCCCTACAAACGGCCAGCAGGGAAATTCTTTGGAAGAAGTGGTCCATG | | |
| | CTGATCAGACCTCCTGCACTTTTGATAACCTGAGTCCCGGCCTGGAGTACAATGTCAGTGT | | |
| | TTACACTGTCAAGGATGACAAGGAAAGTGTCCCTATCTCTGATACCATCATCCCAGCTGTT | | |
| | CCTCCTCCCACTGACCTGCGATTCACCAACATTGGTCCAGACACCATGCGTGTCACCTGGG | | |
| | CTCCACCCCCATCCATTGATTTAACCAACTTCCTGGTGCGTTACTCACCTGTGAAAAATGA | | |
| | GGAAGATGTTGCACAGTTGTCAATTTCTCCTTCAGACAATGCAGTGGTCTTAACAAATCTC | | |
| | CTGCCTGGTACAGAATATGTAGTGAGTGTCTCCAGTGTCTACGAACAACATGAGAGCACAC | | |
| | CTCTTAGAGGAACACAGAAAACAGGTCTTGATTCCCCAACTGGCATTGACTTTTCTGATAT | | |
| | TACTGCCAACTCTTTTACTGTGCACTGGATTGCTCCTCGAGCCACCATCACTGGCTACAGG | | |
| | ATCCGCCATCATCCCGAGCACTTCAGTGGGAGACCTCGAGAAGATCGGGTGCCCCACTCTC | | |
| | ATCCGCCATCATCCCGAGCACTTCAGTCGGAGACCTCGAGAAGATCGGGTGCCCCACTCTC | | |
| | GGAATTCCATCACCCTCACCAACCTCACTCCAGGCACAGAGTATGTGGTCAGCATCGTTGC | | |
| | TCTTAATGGCAGAGAGGAAAGTCCCTTATTGATTGGCCAACAATCAACAGTTTCTGATGTT | | |
| | CCGAGGGACCTGGAAGTTGTTGCTGCGACCCCCACCAGCCTACTGATCAGCTGGGATGCTC | | |
| | CTGCTGTCACAGTGAGATATTACAGGATCACTTACGGAGAAACAGGAGGAAATAGCCCTGT | | |
| | CCAGGACTTCACTGTGCCTGCGACCAAGTCTACAGCTACCATCAGCCCCCTTPAACCTGGA | | |
| | GTTGATTATACCATCACTGTGTATCCTGTCACTGCCCGTGGAGACACCCCCGCAAGCAGCA | | |
| | AGCCAATTTCCATTAATTACCGAACAGAAATTCACAAACCATCCCAGATCCAAGTGACCGA | | |
| | TGTTCAGGACAACAGCATTAGTGTCAAGTGGCTGCCTTCAACTTCCCCTGTTACTGGTTAC | | |
| | AGAGTAACCACCACTCCCAAAAATGCACCAGCACCAACAAAAACTAAAACTGCAGCTCCAG | | |
| | ATCAAACAGAAATGACTATTCAAGCCTTGCAGCCCACAGTGCAGTATGTGGTTAGTGTCTA | | |
| | TGCTCAGAATCCAAGCGGAGAGAGTCAGCCTCTGGTTCAGACTGCAGTAACCAACATTGAT | | |
| | CGCCCTAAAGGACTGGCATTCACTGATGTGGATGTCGATTCCATCAAAATTGCTTCCGAAA | | |
| | GCCCACAGGGGCAAGTTTCCAGGTACACGGTGACCTACTCCAGCCCTCAGGATCGAATCCA | | |
| | TGAGCTATTCCCTGCACCTGATGGTGAAGAAGACACTGCAGACCTCCAAGGCCTCACACCG | | |
| | GGTTCTGAGTACACAGTCAGTGTGGTTCCCTTGCACCATGATATCGACAGCCACCCCCTGA | | |
| | TTGGAACCCAGTCCACAGCTATTCCTGCACCAACTGACCTGAAGTTCACTCAGGTCACACC | | |
| | CACAAGCCTGAGCGCCCAGTGCACACCACCCAATCTTCAGCTCACTGGATATCGAGTGCGG | | |
| | GTGACCCCCAAGGAGAAGACCGGACCAATGAAAGAAATCAACCTTGCTCCTGACAGCTCAT | | |
| | CCGTGGTTGTATCACGACTTATGGTCGCCACCAAATATGAACTGAGTGTCTATGCTCTTAA | | |
| | GGACACTTTGACAAGCAGACCAGCTCAGCGTGTTGTCACCACTCTGGAGAATGTCAGCCCA | | |
| | CCAAGAAGGGCTCGTGTCACAGATGCTACTGAGACCACCATCACCATTAGCTGGAGAACCA | | |
| | AGACTGAGACGATCACTGGCTTCCAAGTTGATGCCGTTCCACCCAATGGCCAGACTCCAAT | | |
| | CCAGAGAACCATCAAGCCAGATGTCAGAAGCTACACCATCACAGGTTTACAACCAGGCACT | | |
| | GACTACAAGATCTACCTGTACACCTTGAATGACAATGCTCGGACCTCCCCTGTGCTCATCG | | |
| | ACGCCTCCACTGCCATTCATGCACCATCCAACCTGCGTTTCCTCCCCACCACACCCAATTC | | |
| | CTTGCTGGTATCATGGCAGCCGCCACGTGCCAGGATTACCGGCTACATCATCAAGTATGAG | | |
| | AAGCCTGGGTCTCCTCCCAGAGAAGTGGTCCCTCGGCCCCCCCCTCGTCTCACACAGGCTA | | |
| | CTATTACTGGCCTGGAACCGCCAACCGAATATACAATTTATGTCATTGCCCTGAAGAATAA | | |
| | TCAGAAGAGCGAGCCCCTGATTGCAAGGAAAAAGACAGACCAGCTTCCCCAACTGGTAACC | | |
| | CTTCCACACCCCAATCTTCATGGACCACAGATCTTGGATGTTCCTTCCACAGTTCAAAAGA | | |
| | CCCCTTTCGTCACCCACCCTGCGTATGACACTGGAAATCGTATTCAGCTTCCTGGCACTTC | | |
| | TGGTCACCAACCCAGTGTTGGGCAACAAATGATCTTTGAGGAACATGGTTTTAGGCGCACC | | |
| | ACACCGCCCACAACGCCCACCCCCATAAGGCATAGGCCAAGACCATICCCGCCGAATGTAG | | |
| | GACAAGAAGCTCTCTCTCAGACAACCATCTCATGGGCCCCATTCCAGGACACTTCTGAGTA | | |
| | CATCATTTCATGTCATCCTGTTCGCACTGATGAAGAACCCTTACAGTTCAGGGTTCCTGCA | | |
| | ACTTCTACCAGTGCCACTCTGACACGCCTCACCAGACGTGCCACCTACAACATCATAGTGG | | |
| | AGGCACTGAAAGACCACCAGAGGCATAAGCTTCCGGAAGACGTTGTTACCGTGGGCAACTC | | |
| | TGTCAACGAAGGCTTGAACCAACCTACGGATGACTCGTGCTTTGACCCCTACACAGTTTCC | | |
| | CATTATGCCGTTGGAGATGAGTGGGAACGAATGTCTGAATCAGGCTTTAAACTGTTGTGCC | | |
| | AGTGCTTAGCCTTTGGAAGTGGTCATTTCAGATGTGATTCATCTAGATGGTGCCATGACAA | | |
| | TGGTGTGAACTACAAGATTGGAGAGAACTGCGACCGTCAGGGAGAA1ATGGCCAGATGATG | | |
| | AGCTGCACATGTCTTGGGAACGGAAAACGAGAATTCAAGTGTGACCCTCATGAGGCAACGT | | |
| | GTTACGATGATGGGAAGACATACCACGTAGGAGAACAGTGGCAGAAGGAATATCTCGGTGC | | |
| | CATTTGCTCCTGCACATGCTTTGGAGGCCAGCGGGGCTGGCGCTGTGACAACTGCCGCAGA | | |
| | CCTGGCGGTGAACCCAGTCCCGAAGGCACTACTGGCCAGTCCTACAACCAGTATTCTCAGA | | |
| | GATACCATCAGAGAACAAACACTAATGTTAATTGCCCAATTGAGTGCTTCATGCCTTTAGA | | |
| | TGTACAGGCTGACAGAGAAGATTCCCGAGAGTAA | | |
| | ORF Start: ATG at 26 | | ORF Stop: TAA at 6986 |
| | SEQ ID NO: 2 | 2320 aa | MW at 255732.8kD |
| NOV1a,CG108 | MVQPQSPVAVSQSKPGCYDNGKNYQINQQWERTYLGNVLVCTCYGGSRGFNCESKPEAEET | | |
| 440-01 Protein | | | |
| Sequence | CFDKYTGNTYRVGDTYERPKDSMIWDCTCIGAGRGRISCTIANRCHEGGQSYKIGDTWRRP | | |
| | HETGGYMLECVCLGNGKGEWTCKPIAEKCFDHAAGTSYVVGETWEKPYQGWMMVDCTCLGE | | |
| | GSGRITCTSRNRCNDQDTRTSYRIGDTWSKKDNRGNLLQCICTGNGRGEWKCERMTSVQTT | | |
| | SSGSGPFTDVRAAVYQPQPHPQPPPYGHCVTDSGVVYSVGMQWLKTQGNKQMLCTCLGNGV | | |
| | SCQETAVTQTYGCNLNGEPCVLPFTYNGRTFYSCTTEGRQDGHLWCSTTSNYEQDQKYSFC | | |
| | TDHTVLVQTQGGNSNGALCHFPFLYNNHNYTDCTSEGRRDNMKWCGTTQNYDADQKFCFCP | | |
| | MAAHEEICTTNEGVMYRIGDQWDKQHDMGHMMRCTCVGNGRCEWTCIAYSQLRDQCIVDDI | | |
| | TYNVNDTFHKRHEEGHMLNCTCFGQGRGRWKCDPVDQCQDSETGTFYQICDSWEKYVHGVP | | |
| | YQCYCYGRGIGEWHCQPLQTYPSSSGPVEVFITETPSQPNSHPIQWNAPQPSHISKYTLRW | | |
| | RPKNSVGRWKEATIPGHLNSYTIKGLKPGVVYEGQLISIQQYGHQEVTRFDFTTTSTSTPV | | |
| | TSNTVTGETTPFSPLVATSESVTEITASSFVVSWVSASDTVSGFRVEYELSEEGDEPQYLD | | |
| | LPSTATSVNIPDLLPGRKYIVNVYQISEDGEQSLILSTSQTTAPDAPPDPTVDQVDDTSIV | | |
| | VRWSRFQAPTTGYRIVYSPSVEGSSTELNLPETANSVTLSDLQPGVQYNITIYAVEENQES | | |
| | TPVVIQQETTGTPRSDTVPSPRDLQFVEVTDVKVTIMWTPPESAVTGYRVDVIPVNLPGEH | | |
| | GQRLPISRNTFAEVTGLSPGVTYYFKVFAVSHGRESKPLTAQQTTKLDAPTNLQFVNETDS | | |
| | TVLVRWTPPRAQITCYRLTVGLTRRGQPRQYNVGPSVSKYPLRNLQPASEYTVSLVAIKGN | | |
| | QESPKATGVFTTLQPGSSIPPYNTEVTETTTVITWTPAPRIGFKLGVRPSQGGEAPREVTS | | |
| | DSGSIVVSGLTPGVEYVYTIQVLRDGQERDAPIVNKVVTPLSPPTNLHLEANPDTGVLTVS | | |
| | WERSTTPDITGYRITTTPTNGQQGNSLEEVVHADQSSCTFDNLSPCLEYNVSVYTVKDDKE | | |
| | SVPISDTIIPAVPPPTDLRFTNIGPDTMRVTWAPPPSIDLTNFLVRYSPVKNEEDVAELST | | |
| | SPSDNAVVLTNLLPGTEYVVSVSSVYEQHESTPLRGRQKTGLDSPTGIDFSDITANSFTVH | | |
| | WIAPRATITGYRIRHHPEHFSGRPREDRVPHSRNSITLTNLTPGTEYVVSIVALNCREESP | | |
| | LLIGQQSTVSDVPRDLEVVAATPTSLLISWDAPAVTVRYYRITYGETGGNSPVQEFTVPGS | | |
| | KSTATISGLKPGVDYTTTVYAVTGRGDSPASSKPISINYRTEIDKPSQMQVTDVQDNSISV | | |
| | KWLPSSSPVTGYRVTTTPKNGPGPTKTKTAGPDQTEMTIEGLQPTVEYVVSVYAQNPSGES | | |
| | QPLVQTAVTNIDRPKGLAFTDVDVDSIKIAWESPQCQVSRYRVTYSSPEDGIHELFPAPDG | | |
| | EEDTAELQGLRPGSEYTVSVVALHDDMESQPLIGTQSTAIPAPTDLKFTQVTPTSLSAQWT | | |
| | PPNVQLTGYRVRVTPKEKTGPMKEINLAPDSSSVVVSCLMVATKYEVSVYALKDTLTSRPA | | |
| | QGVVTTLENVSPPRRARVTDATETTITISWRTKTETITGFQVDAVPANGQTPIQRTIKPDV | | |
| | TSYTITGLQPGTDYKIYLYTLNDNARSSPVVIDASTAIDAPSNLRFLATTPNSLLVSWQPP | | |
| | RARITGYIIKYEKPGSPPREVVPRPRPGVTEATITGLEPGTEYTIYVIALKNNQKSEPLIG | | |
| | RKKTDELPQLVTLPHPNLHGPEILDVPSTVQKTPFVTHPGYDTGNGIQLPGTSGQQPSVGQ | | |
| | QMIFEEHGFRRTTPPTTATPIRHRPRPYPPNVGQEALSQTTISWAPFQDTSEYIISCIPVG | | |
| | TDEEPLQFRVPGTSTSATLTCLTRGATYNTIVEALKDQQRHKVREEVVTVGNSVNEGLNQP | | |
| | TDDSCFDPYTVSHYAVGDEWERMSESGFKLLCQCLGFGSGHFRCDSSRWCHDNGVNYKIGE | | |
| | KWDRQCENCQMMSCTCLGNCKGEFKCDPHEATCYDDCKTYHVGEQWQKEYLGAICSCTCFC | | |
| | GQRGWRCDNCRRPGGEPSPEGTTGQSYNQYSQRYHQRTNTNVNCPIECFMPLDVQADREDS | | |
| | RE | | |
| | SEQ ID NO: 3 | 7361 bp | |
| NOV1b,CG108 | TCAACATGCTTAGGCGTCCGCGGCCCCGCCTGCTCCTCCTCCCCGTCCAGTGCCTGGCGAC | | |
| 440-02 DNA | | | |
| Sequence | AGCGGTGCCCTCCACGGGAGCCTCGAAGAGCAAGAGGCAGGCTCAGCAAATGGTTCAGCCC | | |
| | CAGTCCCCGGTGGCTGTCAGTCAAAGCAAGCCCGGTTGTTATGACAATGGAAAACACTATC | | |
| | AGATAAATCAACAGTGCCACCGGACCTACCTACGCAATCCCTTGCTTTGTACTTCTTATCC | | |
| | AGGAAGCCGAGGTTTTAACTGCGAGAGTAAACCTGAAGCTGAAGAGACTTGCTTTGACAAG | | |
| | TACACTCGCAACACTTACCGAGTGCCTGACACTTATGAGCCTCCTAAAGACTCCATGATCT | | |
| | GGGACTGTACCTGCATCGGGGCTGGGCGAGGGAGAATAAGCTGTACCATCGCAAACCGCTG | | |
| | CCATGAAGGCGGTCACTCCTACAAGATTGGTCACACCTCGACCACACCACATCACACTCCT | | |
| | GGTTACATGTTAGAGTGTGTGTGTCTTGGTAATGGAAAAGGAGAATGGACCTGCAAGCCCA | | |
| | TAGCTGAGAAGTGTTTTGATCATGCTGCTGGGACTTCCTATGTGGTCGGAGAAACGTGGGA | | |
| | GAAGCCCTACCAAGGCTGGATGATGGTAGATTGTACTTGCCTGGGAGAAGGCAGCGGACGC | | |
| | ATCACTTGCACTTCTAGAAATAGATGCAACGATCAGGACACAAGGACATCCTATAGAATTG | | |
| | GAGACACCTGGAGCAAGAAGGATAATCGAGGAAACCTGCTCCAGTGCATCTGCACAGGCAA | | |
| | CGGCCGAGCAGAGTGGAAGTGTGAGACCCACACCTCTGTGCACACCACATCGAGCCCATCT | | |
| | GGCCCCTTCACCCATGTTCGTGCAGCTGTTTACCAACCCCAGCCTCACCCCCAGCCTCCTC | | |
| | CCTATCGCCACTCTCTCACACACACTCCTGTCCTCTACTCTCTCCCCATCCACTCCCTGAA | | |
| | GACACAAGGAAATAAGCAAATGCTTTGCACGTGCCTGGGCAACGGAGTCAGCTGCCAAGAG | | |
| | ACAGCTGTAACCCAGACTTACCCTGGCAACTCAAATCCACACCCATGTCTCTTACCATTCA | | |
| | CCTACAATGGCAGGACGTTCTACTCCTCCACCACACAAGGCCGACACCACGGACATCTTTC | | |
| | GTGCACCACAACTTCGAATTATCACCAGCACCACAAATACTCTTTCTCCACAGACCACACT | | |
| | GTTTTGGTTCAGACTCGACCAGCAAATTCCAATCCTGCCTTCTCCCACTTCCCCTTCCTAT | | |
| | ACAACAACCACAATTACACTGATTGCACTTCTGAGGGCAGAAGAGACAACATGAAGTGGTG | | |
| | TGGGACCACACACAACTATGATGCCCACCACAAGTTTCGGTTCTCCCCCATGGCTCCCCAC | | |
| | GAGGAAATCTGCACAACCAATGAAGGGGTCATGTACCGCATTGGAGATCAGTGGGATAAGC | | |
| | AGCATGACATGGGTCACATGATGAGGTGCACGTGTGTTGGGAATGGTCGTGGGGAATGGAC | | |
| | ATGCATTGCCTACTCGCAGCTTCGAGATCAGTGCATTGTTGATGACATCACTTACAATGTG | | |
| | AACCACACATTCCACAAGCGTCATCAACACCCCCACATPGTGAACTCTACATCCTTCCGTC | | |
| | AGGGTCCCCGCAGGTGGAACTGTGATCCCGTCCACCAATGCCAGGATTCACAGACTGCCAC | | |
| | GTTTTATCAAATTGGACATTCATGGCAGAACTATCTCCATGCTCTCACATACCACTCCTAC | | |
| | TGCTATGGCCGTGGCATTGGGGAGTGGCATTGCCAACCTTTACAGACCTATCCAAGCTCAA | | |
| | GTGGTCCTGTCGAAGTATTTATCACTGAGACTCCGAGTCAGCCCAACTCCCACCCCATCCA | | |
| | GTGGAATGCACCACAGCCATCTCACATTTCCAAGTACATTCTCAGGTGGAGACCTAAAAAT | | |
| | TCTCTAGGCCGTTGGAAGGAACCTACCATACCAGGCCACTTAAACTCCTACACCATCAAAG | | |
| | GCCTGAAGCCTGGTGTGCTATACCACGCCCAGCTCATCAGCATCCAGCAGTACGGCCACCA | | |
| | AGAAGTGACTCCCTTTGACTTCACCACCACCAGCACCAGCACACCTGTGACCAGCAACACC | | |
| | GTGACAGGAGAGACGACTCCCTTTTCTCCTCTTGTGGCCACTTCTGAATCTGTGACCGAAA | | |
| | TCACAGCCAGTAGCTTTCTGGTCTCCTGGGTCTCAGCTTCCGACACCGTGTCGGGATTCCG | | |
| | GGTGGAATATGAGCTGAGTGAGGAGCCACATCAGCCACAGTACCTGGATCTTCCAAGCACA | | |
| | GCCACTTCTGTGAACATCCCTGACCTGCTTCCTGGCCGAAAATACATTCTAAATGTCTATC | | |
| | AGATATCTGAGGATGGGGACCAGAGTTTGATCCTGTCTACTTCACAA1CAACAGCGCCTGA | | |
| | TGCCCCNCCTGACCCGACTCTGGACCAAGTTGATGACACCTCAATTGTTGTTCCCTGGAGC | | |
| | AGACCCCAGGCTCCCATCACAGCGTACAGAATAGTCTATTCGCCATCAGTAGAAGGTAGCA | | |
| | GCACAGAACTCAACCTTCCTGAAACTGCAAACTCCGTCACCCTCAGTGACTTGCAACCTGG | | |
| | TGTTCACTATAACATCACTATCTATGCTCTGGAACAAAATCAAGAAAGTACACCTGTTGTC | | |
| | ATTCAACAAGAAACCACTGGCACCCCACCCTCAGATACAGTGCCCTCTCCCAGGGACCTGC | | |
| | AGTTTGTGGAACTGACAGACGTGAAGCTCACCATCATGTGGACACCGCCTCAGAGTGCAGT | | |
| | GACCCGCTACCGTCTGGATCTGATCCCCGTCAACCTGCCTGCCGAGCACGGCCAGAGGCTG | | |
| | CCCATCAGCAGGAACACCTTTGCAGAAGTCACCGGGCTGTCCCCTGGGCTCACCTATTACT | | |
| | TCAAAGTCTTTGCAGTCAGCCATGGGACGGAGAGCAAGCCTCTGACTGCTCAACAGACAAC | | |
| | CAAACTGGATGCTCCCACTAACCTCCAGTTTGTCAATGAAACTGATTCTACTCTCCTGGTG | | |
| | AGATGGACTCCACCTCGGGCCCAGATAACAGGATACCGACTGACCGTGGGCCTTACCCGAA | | |
| | GAGGNCAGCCCAGGCAGTACAATGTGGGTCCCTCTGTCTCCAAGTACCCNCTGAGGAATCT | | |
| | GCAGCCTGCATCTGAGTACACCGTATCCCTCGTGGCCATAAAGGGCAACCAACAGAGCCCC | | |
| | AAAGCCACTGGAGTCTTTACCACACTGCAGCCTGGGACCTCTATTCCACCTTACAACACCG | | |
| | AGGTGACTGAGACCACCATTGTGATCACATGGACGCCTGCTCCAAGAATTGGTTTTAAGCT | | |
| | GGGTGTACGACCAAGCCAGGGAGGAGAGGCACCACGAGAAGTGACTTCAGACTCAGGAAGC | | |
| | ATCGTTGTGTCCGGCTTGACTCCAGGAGTAGAATACGTCTACACCATCCAAGTCCTGAGAG | | |
| | ATGGACAGGAAAGAGATGCGCCAATTGTAAACAAAGTGGTGACACCATTGTCTCCACCAAC | | |
| | AAACTTGCATCTGGAGGCAAACCCTGACACTGGACTGCTCACAGTCTCCTGGGAGAGGAGC | | |
| | ACCACCCCAGACATTACTGGTTATAGAATTACCACAACCCCTACAAACGGCCAGCAGGGAA | | |
| | ATTCTTTCGAAGAAGTCGTCCATGCTGATCAGAGCTCCTGCACTTTTGATAACCTCAGTCC | | |
| | CGGCCTGGAGTACAATGTCAGTCTTTACACTGTCAAGGATGACAAGGAAACTCTCCCTATC | | |
| | TCTCATACCATCATCCCAGAGCTGCCCCAACTCACTGACCTAAGCTTTGTTCATATAACCG | | |
| | ATTCAAGCATCGGCCTCAGGTCGACCCCGCTAAACTCTTCCACCATTATTGCCTACCGCAT | | |
| | CACAGTAGTTGCCCCAGGAGAAGGTATCCCTATTTTTCAAGATTTTGTGGACTCCTCAGTA | | |
| | GGATACTACACAGTCACAGGGCTGGAGCCGGGCATTGACTATGATATCAGCGTTATCACTC | | |
| | TCATTAATGGCGGCGAGAGTGCCCCTACTACACTGACACAACAAACCGCTGTTCCTCCTCC | | |
| | CACTGACCTGCGATTCACCAACATTGGTCCAGACACCATGCGTGTCACCTGGGCTCCACCC | | |
| | CCATCCATTGATTTAACCAACTTCCTGGTGCGTTACTCACCTGTGAAAAATGAGGAAGATG | | |
| | TTGCAGAGTTGTCAATTTCTCCTTCAGACAATGCAGTGCTCTTAACAAATCTCCTCCCTGC | | |
| | TACAGAATATGTAGTGAGTGTCTCCAGTGTCTACGAACAACATGAGAGCACACCTCTTAGA | | |
| | GGAAGACAGAAAACAGGTCTTGATTCCCCAACTGGCATTGACTTTTCTGATATTACTGCCA | | |
| | ACTCTTTTACTGTGCACTGGATTGCTCCTCGAGCCACCATCACTGGCTACAGGATCCGCCA | | |
| | TCATCCCGAGCACTTCAGTGGGAGACCTCGAGAAGATCGCGTGCCCCACTCTCGGAATTCC | | |
| | ATCACCCTCACCAACCTCACTCCAGGCACAGAGTATCTGGTCAGCATCGTTGCTCTTAATG | | |
| | GCAGAGAGGAAAGTCCCTTATTGATTGGCCAACAATCAACAGTTTCTGATGTTCCCAGCGA | | |
| | CCTGGAAGTTGTTCCTGCGACCCCCACCAGCCTACTGATCAGCTGGGATGCTCCTGCTGTC | | |
| | ACAGTGAGATATTACAGGATCACTTACGGAGAAACAGGAGGAAATAGCCCTGTCCAGGAGT | | |
| | TCACTGTGCCTGGGAGCAAGTCTACAGCTACCATCAGCGGCCTTAAACCTGGAGTTGATTA | | |
| | TACCATCACTGTGTATGCTGTCACTGGCCGTGGAGACAGCCCCGCAAGCAGCAAGCCAATT | | |
| | TCCATTAATTACCGAACAGAAATTGACAAACCATCCCAGATGCAAGTGACCGATGTTCAGG | | |
| | ACAACAGCATTAGTGTCAAGTGGCTCCCTTCAAGTTCCCCTGTTACTGGTTACAGAGTAAC | | |
| | CACCACTCCCAAAAATGGACCAGGACCAACAAAAACTAAAACTGCAGGTCCAGATCAAACA | | |
| | GAAATGACTATTGAAGGCTTGCAGCCCACAGTGGAGTATGTGGTTAGTGTCTATGCTCAGA | | |
| | ATCCAAGCGGAGAGAGTCAGCCTCTGGTTCAGACTGCAGTAACCAACATTGATCGCCCTAA | | |
| | AGGACTGGCATTCACTGATGTGGATGTCGATTCCATCAAAATTGCTTGGGAAAGCCCACAG | | |
| | GGGCAAGTTTCCAGGTACAGGGTGACCTACTCGAGCCCTGAGGATGGAATCCATGACCTAT | | |
| | TCCCTGCACCTGATGGTGAAGAAGACACTGCAGAGCTGCAAGGCCTCAGACCGGGTTCTGA | | |
| | GTACACAGTCAGTGTGGTTGCCTTGCACGATGATATGGAGAGCCAGCCCCTGATTGGAACC | | |
| | CAGTCCACAGCTATTCCTGCACCAACTGACCTGAAGTTCACTCAGGTCACACCCACAAGCC | | |
| | TGAGCGCCCAGTGGACACCACCCAATGTTCAGCTCACTGGATATCGAGTGCGGGTGACCCC | | |
| | CAAGGAGAAGACCGGACCAATGAAAGAAATCAACCTTGCTCCTGACAGCTCATCCGTGGTT | | |
| | GTATCAGGACTTATGGTCGCCACCAAATATCAAGTGAGTGTCTATGCTCTTAAGGACACTT | | |
| | TGACAAGCAGACCAGCTCAGGGNGTTGTCACCACTCTGGAGAATGTCAGCCCACCAAGAAG | | |
| | GGCTCGTGTGACAGATGCTACTGAGACCACCATCACCATTAGCTGGAGAACCAAGACTGAG | | |
| | ACGATCACTGGCTTCCAAGTTGATGCCGTTCCAGCCAATGGCCAGACTCCAATCCAGAGAA | | |
| | CCATCAAGCCAGATGTCAGAAGCTACACCATCACAGGTTTACAACCAGGCACTGACTACAA | | |
| | GATCTACCTGTACACCTTCAATGACAATGCTCGGAGCTCCCCTGTGGTCATCGACGCCTCC | | |
| | ACTGCCATTGATGCACCATCCAACCTGCGTTTCCTGGCCACCACACCCAATTCCTTGCTGG | | |
| | TATCATGGCAGCCGCCACGTGCCAGGATTACCGGCTACATCATCAAGTATGAGAAGCCTGG | | |
| | GTCTCCTCCCACAGAAGTGGTCCCTCGCCCCCGCCCTGGTGTCACAGACGCTACTATTACT | | |
| | GGCCTGGAACCGGGAACCGAATATACAATTTATGTCATTGCCCTGAAGAATAATCAGAAGA | | |
| | GCGAGCCCCTGATTGGAAGGAAAAAGACAGGATGGTGCCATGACAATGGTGTGAACTACAA | | |
| | GATTGGAGAGAAGTGGGACCGTCACGGAGAAAATGGCCAGATGATGAGCTGCACATGTCTT | | |
| | GGGAACGGAAAAGGAGAATTCAAGTGTGACCCTCATGAGGCAACGTGTTATGATGATGGGA | | |
| | AGACATACCACGTAGGAGAACAGTGGCAGAACGAATATCTCGGTGCCATTTGCTCCTGCAC | | |
| | ATGCTTTGGAGGCCAGCGGGGCTGGCGCTGTGACAACTGCCGCAGACCTGGGGGTGAACCC | | |
| | AGTCCCGAAGGCACTACTGGCCAGTCCTACAACCAGTATTCTCAGAGATACCATCAGAGAA | | |
| | CAAACACTAATGTTAATTGCCCAATTGAGTCCTTCATGCCTTTAGATGTACACGCTGACAG | | |
| | AGAAGATTCCCGAGAGTAAATCATCTTTCCAATCCAGAGGAACAAGCATGTCTCTCTGCCA | | |
| | AGATCCATCTAAACTGGAGTGATGTTAGCAGACCCAGCTTAGAGTTCTTCTTTCTTTCTTA | | |
| | AGCCCTTTGCTCTGGAGGAAGTTCTCCAGCTTCAGCTCAACTCACAGCTTCTCCAAGCATC | | |
| | ACCCTGGGAGTTTCCTGAGGGTTTTCTCATAAATGAGGGCTGCACATTGCCTGTTCTGCTT | | |
| | CGAAGTATTCAATACCGCTCAGTATTTTAAATGAAGTGATTCTAAGATTTGGTTTGGGATC | | |
| | AATAGGAAAGCATATGCAGCCAACCAAGATGCAAATGTTTTGAAATGATATGACCAAAATT | | |
| | TTAAGTAGGAAAGTCACCCAAACACTTCTGCTTTCACTTAAGTGTCTGGCCCGCAATACTG | | |
| | TAGGAACAAGCATGATCTTGTTACTGTGATATTTTAAATATCCACACTACTCACTTTTTCC | | |
| | AAATGATCCTAGTAATTGCCTAGAAATATCTTTCTCTTACCTGTTATTTATCAATTTTTCC | | |
| | CAGTATTTTTATACGGAAAAAATTGTATTGAAAACACTTAGTATGCAGTTGATAAGAGGAA | | |
| | TTTGGTATAATTATGGTGGGTGATTATTTTTTATACTGTATGTGCCAAAGCTTTACTACTG | | |
| | TGGAAAGACAACTGTTTTAATAAAAGATTTACATTCCACAA | | |
| | ORF Start: at 3 | | ORF Stop: at 6663 |
| | SEQ ID NO: 4 | 2220 aa | MW at 243994.0kD |

| | |
|---|---|
| NOV1b,CG108 | MLRGPGPGLLLLAVQCLGTAVPSTGASKSKRQAQQMVQPQSPVAVSQSKPGCYDNGKHYQI |
| 440-02 Protein | |
| Sequence | NQQWERTYLGNALVCTCYGGSRGFNCESKPEAEETCFDKYTGNTYRVGDTYERPKDSMIWD |
| | CTCIGAGRGRISCTIANRCHEGGQSYKIGDTWRRPHETGGYMLECVCLGNGKGEWTCKPIA |
| | EKCFDHAAGTSYVVGETWEKPYQGWMMVDCTCLGEGSGRITCTSRNRCNDQDTRTSYRIGD |
| | TWSKKDNRGNLLQCICTGNGRGEWKCERHTSVQTTSSGSGPFTDVRAAVYQPQPHPQPPPY |
| | GHCVTDSGVVYSVGMQWLKTQGNKQMLCTCLGNGVSCQETAVTQTYGGNSNGEPCVLPFTY |
| | NGRTFYSCTTEGRQDGHLWCSTTSNYEQDQKYSFCTDJTVLVQTRGGNSNGALCHFPFLYN |
| | NYNYTDCTSEGRRDNMKWCGTTQNYDADQKFGFCPMAAHEEICTTNEGVMYRIGDQWDKQH |
| | DMGHMMRCTCVGNGRGEWTCIAYSQLRDQCIVDDITYNVNDTFHKRHEEGHMLNCTCFGQG |
| | RGRWKCDPVDQCQDSETGTFYQIGDSWEKYVHGVRYQCYCYGRGIGEWHCQPLQTYPSSSG |
| | PVEVFITETPSQPNSHPIQWNAPQPSHISKYILRWRPKNSVGRWKEATIPGHLNSYTIKGL |
| | KPGVVYEGQLISIQQYGHQEVTRFDFTTTSTSTPVTSNTVTGETTPFSPLVATSESVTEIT |
| | ASSFVVSWVSASDTVSGFRVEYELSEEGDEPQYLDLPSTATSVNIPDLLPGRKYIVNVYQI |
| | SEDGEQSLILSTSQTTAPDAPPDPTVDQVDDTSIVVRWSRPQAPITGYRIVYSPSVEGSST |
| | ELNLPETANSVTLSDLQPGVQYNITIYAVEENQESTPVVIQQETTGTPRSDTVPSPRDLQF |
| | VEVTDVKVTIMWTPPESAVTGYRVDVIPVNLPGEHGQRLPISRNTFAEVTGLSPGVTYYFK |
| | VFAVSHGRESKPLTAQQTTKLDAPTNLQFVNETDSTVLVRWTPPRAQITGYRLTVGLTRRG |
| | QPRQYNVGPSVSKYPLRNLQPASEYTVSLVAIKGNQESPKATGVFTTLQPGSSIPPYNTEV |
| | TETTIVITWTPAPRIGFKLGVRPSQGGEAPREVTSDSGSIVVSGLTPGVEYVYTIQVLRDG |
| | QERDAPIVNKVVTPLSPPTNLHLEANPDTGVLTVSWERSTTPDITGYRITTTPTNGQQGNS |
| | LEEVVHADQSSCTFDNLSPGLEYNVSVYTVKDDKESVPISDTIIPEVPQLTDLSFVDITDS |
| | SIGLRWTPLNSSTIIGYRITVVAAGEGIPIFEDFVDSSVGYYTVTGLEPGIDYDISVITLI |
| | NGGESAPTTLTQQTAVPPPTDLRFTNIGPDTMRVTWAPPPSIDLTNFLVRYSPVKNEEDVA |
| | ELSISPSDNAVVLTNLLPGTEYVVSVSSVYEQHESTPLRGRQKTGLDSPTGIDFSDITANS |
| | FTVHWIAPRATITGYRIRHHPEHFSGRPREDRVPHSRNSITLTNLTPGTEYVVSIVALNGR |
| | EESPLLIGQQSTVSDVPRDLEVVAATPTSLLISWDAPAVTVRYYRITYGETGGNSPVQEFT |
| | VPGSKSTATISGLKPGVDYTITVYAVTGRGDSPASSKPISINYRTEIDKPSQMQVTDVQDN |
| | SISVKWLPSSSPVTGYRVTTTPKNGPGPTKTKTAGPDQTEMTIEGLQPTVEYVVSVVAQNP |
| | SGESQPLVQTAVTNIDRPKGLAFTDVDVDSIKTAWESPQGQVSRYRVTYSSFEDGIHELFP |
| | APDCEEDTAELQGLRPGSEYTVSVVALHDDMESQPLIGTQSTAIPAPTDLKFTQVTPTSLS |
| | AQWTPPNVQLTGYRVRVTPKEKTGPMKEINLAPDSSSVVVSGLMVATKYEVSVYALKDTLT |
| | SRPAQGVVTTLENVSPPRRARVTDATETTITISWRTKTETITCFQVDAVPANCQTPTQRTI |
| | KPDVISYTTTGLQPGTDYKIYLYTLNDNARSSPVVIDASTAIDAPSNLRFLATTPNSLLVS |
| | WQPPRARITGYIIKYEKPGSPPREVVPRPRPGVTEATITGLEPGTEYTIYVIALKNNQKSE |
| | PLICRKKTGWCHDNCVNYKIGEKWDRQGENGQMMSCTCLCNGKGEFKCDPHEATCYDDCKT |
| | YHVCEQWQKEYLCAICSCTCFGGQRCWRCDNCRRPGGEPSPEGTTCQSYNQYSQRYHQRTN |
| | TNVNCPIECFMPLDVQADREDSRE |

Sequence comparison of the above protein sequences yields the following sequence relationships shown in Table 1B.

TABLE 1B
Comparison of NOV1a against NOV1b.

| Protein | NOV1a Residues/ | Identities/Similarities |
|---|---|---|
| Sequence | Match Residues | for the Matched Region |
| NOV1b | 1 . . . 1951 | 1370/1961 (69%) |
| | 36 . . . 1987 | 1496/1961 (75%) |

Three polymorphic variants of NOV1b have been identified and are shown in Table 41A

Further analysis of the NOV1a protein yielded the following properties shown in Table 1C.

TABLE 1C
Protein Sequence Properties NOV1a

| | |
|---|---|
| PSort analysis: | 0.8800 probability located in nucleus; |
| | 0.1695 probability located in lysosome |
| | (lumen); 0.1000 probability located in |
| | mitochondrial matrix space; 0.0000 |
| | probability located in endoplasmic |
| | reticulum (membrane) |
| SignalP analysis: | No Known Signal Sequence Predicted |

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAU74674 | Human fibronectin protein - | 1 . . . 2320 | 2320/2320 (100%) | 0.0 |
| | Homo sapiens, 2324 aa. | 5 . . . 2324 | 2320/2320 (100%) | |
| | [WO200187071-A1, 22- | | | |
| | NOV-2001] | | | |
| AAG68182 | Fibronectin protein SEQ ID | 1 . . . 2320 | 2320/2320 (100%) | 0.0 |
| | NO: 98 - Homo sapiens, | 9 . . . 2328 | 2320/2320 (100%) | |
| | 2328 aa. [WO200177327- | | | |
| | A1, 18-OCT-2001] | | | |
| AAR92778 | Human fibronectin - Homo | 1 . . . 2320 | 2318/2320 (99%) | 0.0 |
| | sapiens, 2324 aa. | 5 . . . 2324 | 2318/2320 (99%) | |
| | [WO9604304-A1, 15-FEB- | | | |
| | 1996] | | | |
| AAP70373 | Human fibronectin gene | 1 . . . 2320 | 2318/2320 (99%) | 0.0 |
| | product - Homo sapiens, | 8 . . . 2327 | 2318/2320 (99%) | |
| | 2327 aa. [EP207751-A, 07- | | | |
| | JAN-1987] | | | |
| AAM38649 | Human polypeptide SEQ ID | 1 . . . 2320 | 2316/2320 (99%) | 0.0 |
| | NO 1794 - Homo sapiens, | 36 . . . 2355 | 2317/2320 (99%) | |
| | 2355 aa. [WO200153312- | | | |
| | A1, 26-JUL-2001] | | | |

In a BLAST search of public sequence datbases, the NOV1a protein was found to have homology to the proteins shown in the BLASTP data in Table 1E.

TABLE 1E
Public BLASTP Results for NOV1a

| | | NOV1a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P02751 | Fibronectin precursor (FN) | 1 . . . 2320 | 2318/2351 (98%) | 0.0 |
| | (Cold-insoluble globulin) | 36 . . . 2386 | 2318/2351 (98%) | |
| | (CIG) - Homo sapiens | | | |
| | (Human), 2386 aa. | | | |
| FNHU | fibronectin precursor | 1 . . . 2320 | 2318/2351 (98%) | 0.0 |
| | [validated] - human, 2386 aa. | 36 . . . 2386 | 2318/2351 (98%) | |
| E981236 | FN PLASMID PFHDEL1 | 1 . . . 1946 | 1703/2026 (84%) | 0.0 |
| | MATURE PROTEIN FROM | 5 . . . 2025 | 1765/2026 (87%) | |
| | PATENT WO9013653 - | | | |
| | vectors, 2231 aa. | | | |
| P07589 | Fibronectin (FN) - Bos | 1 . . . 2183 | 1642/2239 (73%) | 0.0 |
| | taurus (Bovine), 2265 aa. | 5 . . . 2213 | 1786/2239 (79%) | |
| P04937 | Fibronectin precursor (FN) - | 1 . . . 2114 | 1393/2128 (65%) | 0.0 |
| | Rattus norvegicus (Rat), 2477 | 37 . . . 2071 | 1584/2128 (73%) | |
| | aa. | | | |

PFam analysis predicts that the NOV1a protein contains the domains shown in Table 1F.

TABLE 1F
Domain Analysis of NOV1a

| | | Identities/ | |
|---|---|---|---|
| | | Similarities | |
| Pfam | NOV1a | for the Matched | |
| Domain | Match Region | Region | Expect Value |
| fn1 | 17 . . . 52 | 19/41 (46%) | 7.9e−17 |
| | | 35/41 (85%) | |
| fn1 | 62 . . . 100 | 21/41 (51%) | 3.2e−19 |
| | | 39/41 (95%) | |
| fn1 | 106 . . . 144 | 21/41 (51%) | 1.6e−17 |
| | | 36/41 (88%) | |
| fn1 | 151 . . . 190 | 23/41 (56%) | 4.7e−21 |
| | | 37/41 (90%) | |
| fn1 | 196 . . . 235 | 26/41 (63%) | 4.6e−20 |
| | | 38/41 (93%) | |
| fn1 | 273 . . . 307 | 14/41 (34%) | 8.1e−13 |
| | | 31/41 (76%) | |
| fn2 | 325 . . . 366 | 27/42 (64%) | 8e−35 |
| | | 42/42 (100%) | |
| fn2 | 385 . . . 426 | 26/42 (62%) | 4.3e−37 |
| | | 42/42 (100%) | |
| fn1 | 435 . . . 473 | 21/41 (51%) | 4.4e−20 |
| | | 39/41 (95%) | |
| fn1 | 483 . . . 520 | 20/41 (49%) | 2.3e−16 |
| | | 35/41 (85%) | |
| fn1 | 526 . . . 564 | 22/41 (54%) | 1.7e−18 |
| | | 37/41 (90%) | |
| fn3 | 573 . . . 656 | 28/87 (32%) | 1.7e−12 |
| | | 65/87 (75%) | |
| fn3 | 685 . . . 765 | 25/85 (29%) | 1.7e−14 |
| | | 64/85 (75%) | |
| fn3 | 776 . . . 854 | 34/84 (40%) | 1.9e−25 |
| | | 70/84 (83%) | |
| fn3 | 872 . . . 951 | 28/86 (33%) | 5.5e−22 |
| | | 63/86 (73%) | |
| fn3 | 962 . . . 1040 | 27/84 (32%) | 8.7e−21 |
| | | 67/84 (80%) | |
| fn3 | 1052 . . . 1127 | 26/86 (30%) | 0.0035 |
| | | 60/86 (70%) | |
| fn3 | 1139 . . . 1221 | 32/87 (37%) | 5.9e−19 |
| | | 66/87 (76%) | |
| fn3 | 1232 . . . 1312 | 27/85 (32%) | 1.8e−21 |
| | | 69/85 (81%) | |
| fn3 | 1323 . . . 1402 | 32/84 (38%) | 1.9e−22 |
| | | 68/84 (81%) | |
| fn3 | 1413 . . . 1495 | 33/86 (38%) | 4e−27 |
| | | 72/86 (84%) | |
| fn3 | 1507 . . . 1586 | 32/85 (38%) | 4.3e−21 |
| | | 69/85 (81%) | |
| fn3 | 1597 . . . 1676 | 29/86 (34%) | 2.7e−15 |
| | | 63/86 (73%) | |
| fn3 | 1687 . . . 1766 | 31/85 (36%) | 2.6e−20 |
| | | 64/85 (75%) | |
| fn3 | 1779 . . . 1857 | 30/84 (36%) | 1.6e−21 |
| | | 66/84 (79%) | |
| fn3 | 1868 . . . 1947 | 31/86 (36%) | 1.8e−24 |
| | | 69/86 (80%) | |
| fn3 | 2038 . . . 2115 | 25/87 (29%) | 5.2e−06 |
| | | 61/87 (70%) | |
| fn1 | 2140 . . . 2179 | 19/41 (46%) | 3.1e−20 |
| | | 40/41 (98%) | |
| fn1 | 2185 . . . 2222 | 21/41 (51%) | 9.4e−19 |
| | | 37/41 (90%) | |
| fn1 | 2229 . . . 2264 | 18/41 (44%) | 7.6e−16 |
| | | 36/41 (88%) | |

### Example 2

The NOV2 clone was analyzed, and the nucleotide and encoded polypeptide sequences are shown in Table 2A.

TABLE 2A
NOV2 Sequence Analysis

| | SEQ ID NO: 5 | 1309 bp | |
|---|---|---|---|
| NOV2a, | GCCAGGGTTGCCTGCGGGAGCCACGCGTCCGCTCTCCACACCTTTCACAGCCCCACCCCTC | | |
| CG122589-01 | | | |
| DNA Sequence | AGAGCAACCTCAGCCCAGCCCAGCCCAGCTCCAGCTCCAGCTCCAGCCCGGGCCCCATCAT | | |
| | GGCCAAGGACTTTCAAGATATCCAGCAGCTCAGCTCGGAGGAAAATGACCATCCTTTCCAT | | |
| | CAAGGTGAGGGGCCAGGCACTCGCAGGCTGAATCCCAGCACAGGAAATCCATTTTTGAAAG | | |
| | GGCCACCTCCTGCCCAGCCCCTGGCACAGCGTCTCTGCTCCATGGTCTGCTTCAGTCTGCT | | |
| | TGCCCTGAGCTTCAACATCCTGCTGCTGGTGGTCATCTGTGTGACTGGGTCCCAAAGTGAG | | |
| | GGTCACAGAGGTGCACAGCTGCAAGCCGAGCTGCGGAGCCTGAAGGAAGCTTTCAGCAACT | | |
| | TCTCCTCGAGCACCCTGACGGAGGTCCAGGCAATCAGCACCCACGGAGGCAGCGTGGGTGA | | |
| | CAAGATCACATCCCTAGGAGCCAAGCTCGAGAAACAGCAGCAGGACCTGAAAGCAGATCAC | | |
| | GATGCCCTCCTCTTCCATCTCAAGCACTTCCCCGTGGACCTGCGCTTCGTGGCCTCCCAGA | | |
| | TGGAGCTCCTCCACACCAACGGCTCCCAAAGGACCTGCTGCCCCGTCAACTGCGTGGAGCA | | |
| | CCAAGGCAGCTGCTACTGGTTCTCTCACTCCGGGAAGGCCTCGGCTGACCCGGAGAAGTAC | | |
| | TGCCACCTGGAGAACGCACACCTGGTGGTCATCAACTCCTGGGACGAGCAGAAATTCATTG | | |
| | TACAACACACGAACCCCTTCAATACCTGGATACCTCTCACGGACAGTGATGGCTCTTGGAA | | |
| | ATGGGTGGATGGCACAGACTATAGGCACAACTACAAGAACTGGGCTGTCACTCAGCCAGAT | | |
| | AATTGGCACGGGCACGAGCTGGGTGGAAGTGAAGACTGTGTTCAAGTCCAGCCGGATGGCC | | |
| | GCTGGAACGATGACTTCTGCCTGCAGGTGTACCGCTGGGTGTGTGAGAAAAGGCGGAATGC | | |
| | CACCGGCGAGGTGGCCTGACCCCAGCACACCTCTGGCTAACCCATACCCCACACCTGCCCA | | |
| | GCTCTGGCTTCTCTGTTGAGGATTTTGAGGAAAGGAAGAAACACTGAGACAGGGGTATGGG | | |
| | GAAGAGCTGAGCAAAGAGAGAAAGGAGGTAGTTTAAGAGTCCCTGACCCTCCAGGACTGAG | | |
| | ATCCCACCTCCTTCTGTAATTCATTGTAATTATTATAATCGTCAGCCTCTTCAATGGCGTA | | |
| | GGAAAGAAGAAACAAATGCTTGAATCTC | | |
| | ORF Start: ATG at 121 | | ORF Stop: TGA at 1054 |
| | SEQ ID NO: 6 | 311 aa | MW at 35191.1kD |
| NOV2a, | MAKDFQDIQQLSSEENDHPFHQGEGPGTRRLNPRRGNPFLKGPPPAQPLAQRLCSMVCFSL | | |
| CG122589-01 | | | |
| Protein Sequence | LALSFNILLLVVICVTGSQSEGHRCAQLQAELRSLKEAFSNFSSSTLTEVQAISTHGCSVG | | |
| | DKITSLGAKLEKQQQDLKADHDALLFHLKHFPVDLRFVACQMELLHSNGSQRTCCPVNWVE | | |
| | HQGSCYWFSHSGKAWAEAEKYCQLENAHLVVINSWEEQKFTVQHTNPFNTWTCLTDSDGSW | | |
| | KWVDGTDYRHNYKNWAVTQPDNWHGHELGCSEDCVEVQPDGRWNDDFCLQVYRWVCEKRRN | | |
| | ATGEVA | | |
| | SEQ ID NO: 7 | 1112 bp | |
| NOV2b, | GCCAGGGTTGCCTCCGGGAGCCAGGCGTCCGCTCTCCACACCTTTCACAGCCCCAGCCCTC | | |
| CG122589-02 | | | |
| DNA Sequence | AGAGCAACCTCAGCCCAGCCCAGCCCAGCTCCAGCTCCAGCTCCAGCCCGGGCCCCATCAT | | |
| | GGCCAAGGACTTTCAAGATATCCAGCAGCTGAGCTCGGAGGAAAATGACCATCCTTTCCAT | | |
| | CAAGGTGAGGGGCCAGGCACTCGCGGGCTGAATCCCAGGAGAGGAAATCCATTTTTGAAAG | | |
| | GGCCACCTCCTCCCCAGCCCCTGGCACACCGTCTCTGCTCCATGGTCTGCTTCAGTCTGCT | | |
| | TGCCCTGAGCTTCAACATCCTGCTGCTGGTGGTCATCTGTGTGACTGGGTCCCAAAGTGCA | | |
| | CAGCTGCAAGCCGAGCTGCGGAGCCTGAAGGAAGCTTTCAGCAACTTCTCCTCGAGCACCC | | |
| | TGACGGAGGTTCAGGCAATCAGCACCCACGGAGGCAGCGTGGGTGACAAGATCACATCCCT | | |
| | AGGAGCCAAGCTGCAGAAACAGCAGCACGACCTGAAAGCAGATCACGATGCCCTGCTCTTC | | |
| | CATCTGAAGCACTTCCCCGTGGACCTGCGCTTCGTGGCCTGCCAGATGGACCTCCTCCACA | | |
| | GCAACGGCTCCCAAAGGACCTGCTGCCCCGTCAACTGGGTGGAGCACCAAGGCAGCTGCTA | | |
| | CTGGTTCTCTCACTCCGGGAAGGCCTGGGCTGAGGCGGAGAAGTACTGCCTGCTGGAGAAC | | |
| | GCACACCTGGTGGTCATCAACTCCTGGGAGGAGCAGAAATTCATTGTACAACACACGAACC | | |
| | CCTTCAATACCTGGATAGGTCTCACGGACAGTGATGGCTCTTGGAAATGGGTGGATGGCAC | | |
| | AGACTATAGGCACAACTACAAGAACTGGGCTGTCACTCAGCCAGATAATTGGCACGGGCAC | | |
| | GAGCTGGGTGGAAGTGAAGACTGTGTTGAAGTCCAGCCGGATGGCCGCTGGAACGATGACT | | |
| | TCTGCCTGCAGGTGTACCGATGGGTGTGTGAGAAAAGGCGGAATGCCACCGGCGAGGTGGC | | |
| | CTGACCCCAGCACACCTCTGGCTAACCCATACCCCACACCTGCCCAGCTCTGCCTTCTCTC | | |
| | TTGAGGATTTTGAG | | |
| | ORF Start: ATG at 121 | | ORF Stop: TGA at 1039 |
| | SEQ ID NO: 8 | 306 aa | MW at 34540.4kD |
| NOV2b, | MAKDFQDIQQLSSEENDHPFHQGEGPGTRGLNPRRGNPFLKGPPPAQPLAQRLCSMVCFSL | | |
| CG122589-02 | | | |
| Protein Sequence | LALSFNILLLVVICVTGSQSAQLQAELRSLKEAFSNFSSSTLTEVQAISTHGGSVGDKITS | | |
| | LGAKLEKQQQDLKADHDALLFHLKHFPVDLRFVACQMELLHSNGSQRTCCPVNWVEHQGSC | | |
| | YWFSHSGKAWAEAEKYCLLENAHLVVINSWEEQKFIVQHTNPFNTWIGLTDSDGSWKWVDG | | |
| | TDYRHNYKNWAVTQPDNWHGHELGGSEDCVEVQPDGRWNDDFCLQVYRWVCEKRRNATGEV | | |
| | A | | |
| | SEQ ID NO: 9 | 1055 bp | |
| NOV2c, | GCCAGGGTTGCCTGCGGGAGCCAGGCGTCCGCTCTCCACACCTTTCACAGCCCCAGCCCTC | | |
| CG122589-03 | | | |
| DNA Sequence | AGAGCAACCTCAGCCCAGCCCAGCCCAGCTCCACCTCCAGCTCCACCCCGGGCCCCATCAT | | |
| | GGCCAAGGACTTTCAAGATATCCAGCAGCTGAGCTCGGAGGAAAATGACCATCCTTTCCAT | | |
| | CAAGGGCCACCTCCTGCCCAGCCCCTCGCACAGCGTCTCTGCTCCATGCTCTCCTTCAGTC | | |
| | TGCTTGCCCTGAGCTTCAACATCCTGCTGCTGGTGGTCATCTGTGTGACTGGGTCCCAAAG | | |
| | TGCACAGCTGCAAGCCGAGCTGCGGAGCCTGAAGGAAGCTTTCAGCAACTTCTCCTCGAGC | | |
| | ACCCTGACGGAGGTCCAGGCAATCAGCACCCACGGAGGCAGCGTGGGTGACAAGATCACAT | | |
| | CCCTAGGAGCCAAGCTGGAGAAACAGCAGCAGGACCTGAAAGCAGATCACGATGCCCTGCT | | |
| | CTTCCATCTGAAGCACTTCCCCGTGGACCTGCGCTTCGTGGCCTGCCAGATGGAGCTCCTC | | |
| | CACAGCAACGGCTCCCAAAGGACCTGCTGCCCCGTCAACTGGGTGGAGCACCAAGGCAGCT | | |
| | GCTACTGGTTCTCTCACTCCGGGAAGGCCTGGGCTGAGGCGGAGAAGTACTGCCAGCTGCA | | |
| | GAACGCACACCTGGTGGTCATCAACTCCTGGGAGGAGCAGAAATTCATTGTACAACACACG | | |
| | AACCCCTTCAATACCTGGATAGCTCTCACGCACACTGATGGCTCTTGGAAATGCCTGGATG | | |
| | GCACAGACTATAGGCACAACTACAAGAACTGGGCTGTCACTCAGCCAGATAATTGGCACGG | | |
| | GCACGAGCTGCGTGGAAGTGAAGACTGTGTTGAAGTCCAGCCGGATGGCCGCTCGAACGAT | | |
| | GACTTCTGCCTGCAGGTGTACCGCTGGGTGTGTGAGAAAAGGCGGAATGCCACCGCCGAGG | | |
| | TGGCCTGACCCCAGCACACCTCTGGCTAACCCATACCCCACACCTGCCCAGCTCTGGCTTC | | |
| | TCTGTTGAGGATTTTGAG | | |
| | ORF Start: ATG at 121 | | ORF Stop: TGA at 982 |
| | SEQ ID NO: 10 | 287 aa | MW at 32550.1kD |

| | |
|---|---|
| NOV2c, | MAKDFQDIQQLSSEENDHPFHQGPPPAQPLAQRLCSMVCFSLLALSFNILLLVVICVTGSQ |
| CG122589-03 | |
| Protein Sequence | SAQLQAELRSLKEAPSNFSSSTLTEVQATSTHGGSVGDKITSLGAKLEKQQQDLKADHDAL |
| | LFHLKHFPVDLRFVACQMELLHSNGSQRTCCPVNWVEHQCSCYWFSHSGKAWAEAEKYCQL |
| | ENAHLVVINSWEEQKFIVQHTNPFNTWIGLTDSDGSWKWVDGTDYRHNYKNWAVTQPDNWH |
| | GHELGGSEDCVEVQPDGRWNDDFCLQVYRWVCEKRRNATGEVA |

Sequence comparison of the above protein sequences yields the following sequence relationships shown in Table 2B.

TABLE 2B
Comparison of NOV2a against NOV2b and NOV2c.

| Protein | NOV2a Residues/ | Identities/Similarities |
|---|---|---|
| Sequence | Match Residues | for the Matched Region |
| NOV2b | 1 . . . 311 | 291/311 (93%) |
| | 1 . . . 306 | 291/311 (93%) |
| NOV2c | 1 . . . 311 | 274/311 (88%) |
| | 1 . . . 287 | 274/311 (88%) |

Further analysis of the NOV2a protein yielded the following properties shown in Table 2C.

TABLE 2C
Protein Sequence Properties NOV2a

| | |
|---|---|
| PSort analysis: | 0.7900 probability located in plasma |
| | membrane; 0.7060 probability located in |
| | microbody (peroxisome); 0.3000 probability |
| | located in Golgi body; 0.2000 |
| | probability located in endoplasmic reticulum |
| | (membrane) |
| SignalP analysis: | Cleavage site between residues 3 and 4 |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW15246 | Asialoglycoprotein receptor | 1 . . . 311 | 287/311 (92%) | e−171 |
| | L-H2 - Homo sapiens, 287 aa. | 1 . . . 287 | 287/311 (92%) | |
| | [EP773289-A2, 14-MAY- | | | |
| | 1997] | | | |
| AAW15252 | Asialoglycoprotein receptor | 1 . . . 311 | 270/311 (86%) | e−159 |
| | L-H2 | 1 . . . 270 | 270/311 (86%) | |
| | cytoplasmic + extracellular | | | |
| | domains - Chimeric Homo | | | |
| | sapiens, 270 aa. [EP773289- | | | |
| | A2, 14-MAY-1997] | | | |
| AAW15251 | Asialoglycoprotein receptor | 83 . . . 311 | 226/229 (98%) | e−140 |
| | L-H2 extracellular domain - | 1 . . . 229 | 227/229 (98%) | |
| | Chimeric Homo sapiens, 229 | | | |
| | aa. [EP773289-A2, 14-MAY- | | | |
| | 1997] | | | |
| AAW15245 | Asialoglycoprotein receptor | 1 . . . 301 | 173/301 (57%) | e−103 |
| | H1 - Homo sapiens, 291 aa. | 1 . . . 278 | 214/301 (70%) | |
| | [EP773289-A2, 14-MAY- | | | |
| | 1997] | | | |
| AAW15250 | Asialoglycoprotein receptor | 1 . . . 301 | 162/301 (53%) | 1e−95 |
| | H1 cytoplasmic + extracellular | 1 . . . 261 | 200/301 (65%) | |
| | domains - Chimeric Homo | | | |
| | sapiens, 274 aa. [EP773289- | | | |
| | A2, 14-MAY-1997] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P07307 | Asialoglycoprotein receptor 2 | 1 . . . 311 | 311/311 (100%) | 0.0 |
| | (Hepatic lectin H2) (ASGP- | 1 . . . 311 | 311/311 (100%) | |
| | R) (ASGPR) - Homo sapiens | | | |
| | (Human), 311 aa. | | | |
| P24721 | Asialoglycoprotein receptor 2 | 1 . . . 307 | 198/307 (64%) | e−114 |
| | (Hepatic lectin 2) (MHL-2) | 1 . . . 300 | 225/307 (72%) | |
| | (ASGP-R) (ASGPR) - Mus | | | |
| | musculus (Mouse), 301 aa. | | | |
| LNRT2 | hepatic lectin 2 - rat, 301 aa. | 1 . . . 307 | 191/307 (62%) | e−112 |
| | | 1 . . . 300 | 225/307 (73%) | |
| P08290 | Asialoglycoprotein receptor | 1 . . . 307 | 189/307 (61%) | e−109 |
| | R2/3 (Hepatic lectin 2/3) | 1 . . . 300 | 223/307 (72%) | |
| | (RHL-2) (ASGP-R) | | | |
| | (ASGPR) - Rattus norvegicus | | | |
| | (Rat), 301 aa. | | | |
| AAH32130 | Asialoglycoprotein receptor 1 - | 1 . . . 301 | 173/301 (57%) | e−103 |
| | Homo sapiens (Human), | 1 . . . 278 | 213/301 (70%) | |
| | 291 aa. | | | |

PFam analysis predicts that the NOV2a protein contains the domains shown in Table 2F.

TABLE 2F
Domain Analysis of NOV2a

| | | Identities/ | |
|---|---|---|---|
| Pfam | NOV2a | Similarities for | Expect |
| Domain | Match Region | the Matched Region | Value |
| lectin_c | 194 . . . 302 | 51/127 (40%) | 9e−50 |
| | | 99/127 (78%) | |

### Example 3

The NOV3 clone was analyzed, and the nucleotide and encoded polypeptide sequences are shown in Table 3A.

TABLE 3A
NOV3 Sequence Analysis

| | SEQ ID NO: 11 | 3934 bp | |
|---|---|---|---|
| NOV3a, | TCCAGTAAGGAGTCGGGGTCTTCCCCAGTTTTCTCAGCCACCCCGCGGCGGCGACTCGCAA | | |
| CG133274-01 | | | |
| DNA Sequence | TGTTTGGCCTCAAAAGAAACGCGGTAATCGGACTCAACCTCTACTCTGGGGGGGCCGGCTT | | |
| | GGGGGCCGGCAGCGGCGGCGCCACCCGCCCGGGAGGGCGACTTTTGGCTACGGAGAAGGAG | | |
| | GCCTCGGCCCGGCGAGAGATAGGGGGAGGGGAGGCCGGCGCGGTGATTGGCGGAAGCGCCG | | |
| | GCGCAAGCCCCCCGTCCACCCTCACGCCAGACTCCCGGAGGGTCGCGCGGCCCCCGCCCAT | | |
| | TGGCGCCGAGGTCCCCGACGTCACCGCGACCCCCGCGAGGCTGCTTTTCTTCGCGCCCACC | | |
| | CGCCGCGCGGCGCCGCTTGAGGAGATGGAAGCCCCGGCCGCTGACGCCATCATGTCGCCCG | | |
| | AAGAGGAGCTGGACGCGTACGAGCCGGAGCCTCTCGGGAAGCGGCCGGCTGTCCTGCCGCT | | |
| | GCTGGACTTGGTCGGGGAATCTGGTAATAACACCAGTACGGACGGGTCACTACCCTCGACG | | |
| | CCGCCGCCAGCAGAGGAGGAGCAGGACGAGTTGTACCGGCAGTCGCTGGAGATTATCTCTC | | |
| | GGTACCTTCGGGAGCAGGCCACCGGCGCCAAGGACACAAAGCCAATGGGCAGGTCTGGGGC | | |
| | CACCAGCAGGAAGGCGCTGGAGACCTTACGACGGGTTGGGGATGGCGTGCAGCGCAACCAC | | |
| | GAGACGGTCTTCCAAGGCATGCTTCGGAAACTGGACATCAAAAACGAAGACGATGTGAAAT | | |
| | CGTTGTCTCGAGTGATGATCCATGTTTTCAGCGACGGCGTAACAAACTGGCGCAGGATTGT | | |
| | GACTCTCATTTCTTTTGGTGCCTTTGTGGCTAAACACTTGAAGACCATAAACCAAGAAAGC | | |
| | TGCATCGAACCATTAGCAGAAAGTATCACAGACGTTCTCGTAAGGACAAAACGGGACTGGC | | |
| | TAGTTAAACAAAGAGGCTGGGATGGGTTTGTGGAGTTCTTCCATGTAGAGGACCTAGAAGG | | |
| | TGCCATCAGGAATGTGCTCCTGGCTTTTGCAGGTGTTGCTGGAGTAGGAGCTGGTTTGCCA | | |
| | TATCTAATAAGATAGCCTTACTGTAAGTGCAATAGTTGACTTTTAACCAACCACCACCACC | | |
| | ACCAAAACCAGTTTATGCAGTTGGACTCCAAGCTGTAACTTCCTAGAGTTGCACCCTAGCA | | |
| | ACCTAGCCAGAAAAGCAAGTGGCAAGAGGATTATGGCTAACAAGAATAAATACATGGGAAG | | |
| | AGTGCTCCCCATTGATTGAAGAGTCACTGTCTGAAAGAAGCAAAGTTCAGTTTCAGCAACA | | |
| | AACAAACTTTGTTTGGGAAGCTATGGAGGAGGACTTTTAGATTTAGTGAAGATGGTAGGGT | | |
| | GGAAAGACTTAATTTCCTTGTTGAGAACAGGAAAGTGGCCAGTAGCCAGGCAAGTCATAGA | | |
| | ATTGATTACCCGCCGAATTCATTAATTTACTGTAGTAGTGTTAAGAGAAGCACTAAGAATG | | |
| | CCAGTGACCTGTGTAAAAGTTACAAGTAATAGAACTATGACTGTAAGCCTCAGTACTGTAC | | |
| | AAGGGAAGCTTTTCCTCTCTCTAATTAGCTTTCCCAGTATACTTCTTAGAAAGTCCAAGTG | | |
| | TTCAGGACTTTTATACCTCTTATACTTTGGCTTGGTTCCATGATTCTTACTTTATTAGCCT | | |
| | AGTTTATCACCAATAATACTTGACGGAAGGCTCAGTAATTAGTTATGAATATGGATATCCT | | |
| | CAATTCTTAAGACAGCTTGTAAATGTATTTGTAAAAATTGTATATATTTTTACAGAAAGTC | | |
| | TATTTCCTTGAAACGAAGGAAGTATCGAATTTACATTAGTTTTTTTCATACCCTTTTGAAC | | |
| | TTTGCAACTTCCGTAATTAGGAACCTGTTTCTTACAGCTTTTCTATGCTAAACTTTGTTCT | | |
| | GTTCAGTTCTAGAGTGTATACAGAACGAATTGATGTGTAACTGTATGCAGACTGGTTGTAG | | |
| | TGGAACAAATCTGATAACTATGCAGGTTTAAATTTTCTTATCTGATTTTGGTAAGTATTCC | | |
| | TTAGATAGGTTTTCTTTGAAAACCTGGGATTGAGAGGTTGATGAATGGAAATTCTTTCACT | | |
| | TCATTATATGCAAGTTTTCAATAATTAGGTCTAAGTGGAGTTTTAAGGTTACTGATGACTT | | |
| | ACAAATAATGGGCTCTGATTGGGCAATACTCATTTGAGTTCCTTCCATTTGACCTAATTTA | | |
| | ACTGGTGAAATTTAAAGTGAATTCATGGGCTCATCTTTAAAGCTTTTACTAAAAGATTTTC | | |
| | AGCTGAATGGAACTCATTAGCTGTGTGCATATAAAAAGATCACATCAGGTGGATGCAGAGA | | |
| | CATTTCATCCCTTGTTTCCTTAATAAATTATAAAATGATCGCTTGGAAAAGCAGCCTAGTC | | |
| | TAACCATGGTGCTATTATTAGGCTTGCTTGTTACACACACAGGTCTAAGCCTAGTATGTCA | | |
| | ATAAAGCAAATACTTACTGTTTTGTTTCTATTAATGATTCCCAAACCTTGTTGCAAGTTTT | | |
| | TGCATTGGCATCTTTGGATTTCAGTCTTGATGTTTGTTCTATCAGACTTAACCTTTTATTT | | |
| | CCTGTCCTTCCTTGAAATTGCTGATTGTTCTGCTCCCTCTACAGATATTTATATCAATTCC | | |
| | TACAGCTTTCCCCTGCCATCCCTGAACTCTTTCTAGCCCTTTTAGATTTTGGCACTGTCAA | | |
| | ACCCCTGCTGGAAACCTGAGTGACCCTCCCTCCCCACCAAGAGTCCACAGACCTTTCATCT | | |
| | TTCACGAACTTGATCCTGTTACCAGGTGGTAATACCATGGGTGCTGTGACACTAACAGTCA | | |
| | TTGAGACGTGCGACGAAGTCCCTTTTCCTTGGACTCGTATCTTTTCAACTATTGTTTTATC | | |
| | CTGTCTTTGGCGGCAATGTGTCAAAAGTCCCCTCAGGAATTTTCAGAGCAAAGAACATTTT | | |
| | ATGAGGCTTTCTCTAAAGTTTCCTTTGTATAGGAGTATGCTCACTTAAATTTACAGAAAGA | | |
| | GGTGAGCTGTGTTAAACCTCAGACTTTAAAAGCTACTGATAAACTGAAGAAAGTGTCTATA | | |
| | TTGGAACTAGGGTCATTTGAAAGCTTCAGTCTCGGAACATGACCTTTAGTCTGTGGACTCC | | |
| | ATTTAAAAATAGGTATGAATAAGATGACTAAGAATGTAATGGGGAAGAACTGCCCTGCCTG | | |
| | CCCATCTCAGAGCCATAAGGTCATCTTTGCTAGAGCTATTTTTACCTATGTATTTATCGTT | | |
| | CTTGATCATAAGCCGCTTATTTATATCATGTATCTCTAAGGACCTAAAAGCACTTTATGTA | | |
| | GTTTTTAATTAATCTTAAGATCTGGTTACGGTAACTAAAAGCCTGTCTGCCAAATCCAGTG | | |
| | GAAACAAGTGCATAGATGTGAATTGGTTTTTAGGGGCCCCACTTCCCAATTCATTAGGTAT | | |
| | GACTGTGGAAATACAGACAACGACTTAGTTCATATTTTGGGCTTGCGGCAGTCAGGGCTTA | | |
| | GGACACCCCPAGTGGTTTGGCAAAGGAGGAGGGAGTGGTGGGTTTATAGGGGAGGAGGAGG | | |
| | CAGGTGGTCTAAGTGCTGACTGGCTACGTAGTTCGGGCAAATCCTCCAAAAGGGAAAGGGA | | |
| | GGATTTGCTTAGAAGGATGGGGCTCCCAGTGACTACTTTTTGACTTCTGTTTGTCTTACGC | | |
| | TTCTCTCAGGGAAAAACATGCAGTCCTCTAGTGTTTCATGTACATTCTGTGGGGGGTGAAC | | |
| | ACCTTGGTTCTGGTTAAACAGCTGTACTTTTGATAGCTGTGCCAGGAAGGGTTAGGACCAA | | |
| | CTACAAATTAATCTTGGTTCTCAAATGTAGTGTGTTTCCCTAACTTTCTCTTTTTCCTGAG | | |
| | AAAAAAAAATAAATCTTTTATTCAAATAAA | | |
| | ORF Start: ATG at 61 | | ORF Stop: TAG at 1111 |
| | SEQ ID NO: 12 | 350 aa | MW at 37364.9kD |
| NOV3a, | MFGLKRNAVIGLNLYCGGACLGAGSGGATRPCGRLLATEKEASARREIGGGEAGAVIGGSA | | |
| CG133274-01 | | | |
| Protein | GASPPSTLTPDSRRVARPPPIGAEVPDVTATPARLLFFAPTRRAAPLEEMEAPAADAIMSP | | |
| Sequence | | | |
| | EEELDGYEPEPLGKRPAVLPLLELVGESGNNTSTDGSLPSTPPPAEEEEDELYRQSLEIIS | | |
| | RYLREQATGAKDTKPMGRSGATSRKALETLRRVGDGVQRNHETVFQGMLRKLDIKNEDDVK | | |
| | SLSRVMINVFSDGVTNWGRIVTLISFCAFVAKHLKTINQESCIEPLAESITDVLVRTKRDW | | |
| | LVKQRGWDGFVEFFHVEDLEGCIRNVLLAFAGVAGVGACLAYLIR | | |
| | SEQ ID NO: 13 | 724 bp | |
| NOV3b, | ATGTTTGGCCTCAAAAGAAACGCGGTAATCGGACTCAACCTCTACTGTGGGGGGGCCGGCT | | |
| CG133274-02 | | | |
| DNA Sequence | TGGGGGCCGGCAGCGGCGGCGCCACCCGCCCGGGAGGGCGACTTTTGGCTACGGAGAAGGA | | |
| | GGCCTCGGCCCGGCGAGAGATAGGGGGAGGGGAGGCCGGCGCGGTGATTGGCGCCAAGGAC | | |
| | ACAAAGCCAATGGGCAGGTCTGGGGCCACCAGCAGGAAGGCGCTGGAGACCTTACGACGGG | | |
| | TTGGGGATGGCGTGCAGCGCAACCACGAGACGGCCTTCCAAGGCATGCTTCGGAAACTGGA | | |
| | CATCAAAAACGAAGACGATGTGAAATCGTTGTCTCGAGTGATGATCCATGTTTTCAGCGAC | | |
| | GGCGTAACAAACTGGGGCAGGATTGTGACTCTCATTTCTTTTGGTGCCTTTGTGGCTAAAC | | |
| | ACTTGAAGACCATAAACCAAGAAAGCTGCATCGAACCATTAGCAGAAAGTATCACAGACGT | | |
| | TCTCGTAAGGACAAAACGCGACTGGCTAGTTAAACAAAGAGGCTGGCATGGGTTTGTGGAG | | |
| | TTCTTCCATGTACACGACCTAGAAGGTGGCATCAGGAATGTGCTCCTGGCTTTTGCAGGTG | | |
| | TTGCTGGAGTAGGAGCTGGTTTGGCATATCTAATAAGATAGCCTTACTGTAAGTGCGATAG | | |
| | TTGACTTTTAACCAACCACCACCACCACCAAAACCAGTTTATGCAGTTGGACT | | |
| | ORF Start: ATG at 1 | | ORF Stop: TAG at 649 |
| | SEQ ID NO: 14 | 216 aa | MW at 23108.3kD |
| NOV3b, | MFGLKRNAVIGLNLYCGGAGLGAGSGGATRPGGRLLATEKEASARREIGGGEAGAVIGAKD | | |
| CG133274-02 | | | |
| Protein | TKPMGRSGATSRKALETLRRVGDGVQRNHETAFQGMLRKLDIKNEDDVKSLSRVMIHVFSD | | |
| Sequence | | | |
| | GVTNWGRIVTLISFGAFVAKHLKTINQESCTEPLAESITDVLVRTKRDWLVKQRGWDGFVE | | |
| | FFHVEDLEGGIRNVLLAFACVACVGACLAYLIR | | |
| | SEQ ID NO: 15 | 667 bp | |
| NOV3c, | CACCGGATCCATGTTTGCCCTCAAAAGAAACGCGGTAATCGGACTCAACCTCTACTGTCGC | | |
| 278876765 | | | |
| DNA Sequence | GGGGCCGGCTTGGGGGCCGGCACCGGCCGCGCCACCCGCCCGGGAGGGCCACTTTTGGCTA | | |
| | CGCAGAAGGAGGCCTCGGCCCGGCGACACATAGGGGGAGGGGAGGCCGCCGCCGTGATTGG | | |
| | CGCCAAGGACACAAAGCCAATGGGCAGGTCTGGGGCCACCAGCAGGAAGCCGCTGGAGACC | | |
| | TTACGACGGGTTGGGGATGGCCTGCAGCGCAACCACCAGACGGCCTTCCAAGGCATGCTTC | | |
| | GGAAACTGGACATCAAAAACCAAGACGATGTGAAATCGTTCTCTCGAGTCATCATCCATGT | | |
| | TTTCAGCGACGGCGTAACAAACTGGGGCAGGATTGTGACTCTCATTTCTTTTGGTGCCTTT | | |
| | GTGGCTAAACACTTGAAGACCATAAACCAAGAAACCTCCATCGAACCATTAGCAGAAAGTA | | |
| | TCACAGACGTTCTCGTAAGGACAAAACGGGACTGGCTAGTTAAACAAAGAGGCTGGGATGG | | |
| | GTTTGTCGAGTTCTTCCATGTAGAGCACCTAGAAGGTCGCATCAGGAATGTGCTGCTGGCT | | |
| | TTTGCAGGTGTTGCTGGAGTAGGAGCTGGTTTGGCATATCTAATAAGAGTCGACGGC | | |
| | ORF Start: at 2 | | ORF Stop: end of sequence |
| | SEQ ID NO: 16 | 222 aa | MW at 23624.8kD |
| NOV3c, | TGSMFGLKRNAVIGLNLYCGGAGLGAGSGGATRPGGRLLATEKEASARREIGGGEAGAVTG | | |
| 278876765 | | | |
| Protein | AKDTKPMGRSGATSRKALETLRRVGDGVQRNHETAFQGMLRKLDIKNEDDVKSLSRVMIHV | | |
| Sequence | | | |
| | FSDGVTNWGRIVTLISFGAFVAKHLKTTNQESCIEPLAESTTDVLVRTKRDWLVKQRGWDG | | |
| | FVEFFHVEDLEGGIRNVLLAFAGVAGVGAGLAYLIRVDG | | |
| | SEQ ID NO: 17 | 610 bp | |
| NOV3d, | CACCGGATCCGGCTTGGGGGCCGGCAGCGGCGGCGCCACCCGCCCGGGAGGGCGACTTTTG | | |
| 278881214 | | | |
| DNA Sequence | GCTACGGAGAAGGAGGCCTCGGCCCGGCCACAGATAGGCCGAGGGGAGCCCGGCGCGGTGA | | |
| | TTGGCGCCAAGGACACAAACCCAATGGCCACGTCTGGGGCCACCAGCAGGAAGCCGCTGGA | | |
| | GACCTTACGACGGGTTGGGGATGGCGTGCAGCGCAACCACGAGACGGCCTTCCAAGGCATG | | |
| | CTTCGGAAACTGGACATCAAAAACGAAGACGATGTGAAATCGTTGTCTCGAGTGATGATCC | | |
| | ATGTTTTCACCGACGCCGTAACAAACTCG3CCAGGATTCTGACTCTCATTTCTTTTGGTGC | | |
| | CTTTGTGGCTAAACACTTGAAGACCATAAACCAAGAAAGCTGCATCCAACCATTACCAGAA | | |
| | AGTATCACAGACGTTCTCGTAAGGACAAAACGGGACTGGCTAGTTAAACAAAGAGGCTGGG | | |
| | ATGGGTTTGTGGAGTTCTTCCATGTAGAGGACCTAGAAGGTGGCATCAGGAATGTGCTGCT | | |
| | GGCTTTTGCAGGTCTTGCTGGACTAGGAGCTCCTTTGGCATATCTAATAAGAGTCGACGGC | | |
| | ORF Start: at 2 | | ORF Stop: end of sequence |
| | SEQ ID NO: 18 | 203 aa | MW at 21645.5kD |

| | |
|---|---|
| NOV3d, | TGSGLGAGSGGATRPGGRLLATEKEASARREIGGGEAGAVIGAKDTKPMGRSGATSRKALE |
| 278881214 | |
| Protein | TLRRVGDCVQRNHETAFQGMLRKLDIKNEDDVKSLSRVMIHVFSDGVTNWGRIVTLISFGA |
| Sequence | |
| | FVAKHLKTINQESCIEPLAESITDVLVRTKRDWLVKQRCWDGFVEFFHVEDLEGGIRNVLL |
| | AFAGVAGVGAGLAYLIRVDG |

Sequence comparison of the above protein sequences yields the following sequence relationships shown in Table 3B.

TABLE 3B
Comparison of NOV3a against NOV3b through NOV3d.

| Protein | NOV3a Residues/ | Identities/Similarities |
|---|---|---|
| Sequence | Match Residues | for the Matched Region |
| NOV3b | 194 . . . 350 | 140/157 (89%) |
| | 60 . . . 216 | 140/157 (89%) |
| NOV3c | 194 . . . 350 | 140/157 (89%) |
| | 63 . . . 219 | 140/157 (89%) |
| NOV3d | 194 . . . 350 | 140/157 (89%) |
| | 44 . . . 200 | 140/157 (89%) |

Further analysis of the NOV3a protein yielded the following properties shown in Table 3C.

TABLE 3C
Protein Sequence Properties NOV3a

| | |
|---|---|
| PSort analysis: | 0.7300 probability located in plasma |
| | membrane; 0.6400 probability located in |
| | endoplasmic reticulum (membrane); |
| | 0.1000 probability located in |
| | endoplasmic reticulum (lumen); |
| | 0.1000 probability located in outside |
| SignalP analysis: | Cleavage site between residues 20 and 21 |

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAE02462 | Human Mcl-1 protein - | 1 . . . 350 | 350/350 (100%) | 0.0 |
| | Homo sapiens, 350 aa. | 1 . . . 350 | 350/350 (100%) | |
| | [WO200136594-A1, 25- | | | |
| | MAY-2001] | | | |
| AAR68814 | Human mcl-1 gene product - | 1 . . . 350 | 349/350 (99%) | 0.0 |
| | Homo sapiens, 350 aa. | 1 . . . 350 | 349/350 (99%) | |
| | [WO9429330-A, 22-DEC- | | | |
| | 1994] | | | |
| ABB57224 | Mouse ischaemic condition | 1 . . . 350 | 266/350 (76%) | e−144 |
| | related protein sequence SEQ | 1 . . . 331 | 289/350 (82%) | |
| | ID NO: 570 - Mus musculus, | | | |
| | 331 aa. [WO200188188-A2, | | | |
| | 22-NOV-2001] | | | |
| AAE02463 | Human Mcl-1s/deltaTM | 1 . . . 230 | 230/230 (100%) | e−129 |
| | variant protein - Homo | 1 . . . 230 | 230/230 (100%) | |
| | sapiens, 271 aa. | | | |
| | [WO200136594-A1, 25- | | | |
| | MAY-2001] | | | |
| AAU76554 | Murine Bcl-2 polypeptide - | 193 . . . 319 | 45/139 (32%) | 2e−08 |
| | Mus sp, 236 aa. | 66 . . . 199 | 65/139 (46%) | |
| | [WO200205835-A2, 24- | | | |
| | JAN-2002] | | | |

In a BLAST search of public sequence datbases, the NOV3a protein was found to have homology to the proteins shown in the BLASTP data in Table 3E.

TABLE 3E
Public BLASTP Results for NOV3a

| | | NOV3a | | |
|---|---|---|---|---|
| Protein | | Residues/ | Identities/ | |
| Accession | | Match | Similarities for the | Expect |
| Number | Protein/Organism/Length | Residues | Matched Portion | Value |
| A47476 | BCL2 homolog MCL1 - human, | 1 . . . 350 | 350/350 (100%) | 0.0 |
| | 350 aa. | 1 . . . 350 | 350/350 (100%) | |
| Q9UNJ1 | Myeloid cell differentiation | 1 . . . 350 | 349/350 (99%) | 0.0 |
| | protein (Myeloid cell | 1 . . . 350 | 349/350 (99%) | |
| | leukemia protein 1) (Myeloid | | | |
| | cell leukemia sequence 1) | | | |
| | (BCL2-related) - Homo | | | |
| | sapiens (Human), 350 aa. | | | |
| Q07820 | Induced myeloid leukemia | 1 . . . 350 | 348/350 (99%) | 0.0 |
| | cell differentiation protein | 1 . . . 350 | 349/350 (99%) | |
| | Mcl-1 - Homo sapiens (Human), 350 aa. | | | |
| Q9Z1P3 | Mcl-1 protein - Rattus | 1 . . . 350 | 271/350 (77%) | e−144 |
| | norvegicus (Rat), 330 aa. | 1 . . . 330 | 286/350 (81%) | |
| P97287 | EAT/MCL-1 protein (MCL1) | 1 . . . 350 | 266/350 (76%) | e−144 |
| | (Myeloid cell leukemia | 1 . . . 331 | 289/350 (82%) | |
| | sequence 1) - Mus musculus | | | |
| | (Mouse), 331 aa. | | | |

PFam analysis predicts that the NOV3a protein contains the domains shown in Table 3F.

TABLE 3F
Domain Analysis of NOV3a

| | | Identities/ | |
|---|---|---|---|
| Pfam | NOV3a | Similarities for | Expect |
| Domain | Match Region | the Matched Region | Value |
| Bcl-2 | 213 . . . 312 | 35/108 (32%) | 1.3e−46 |
| | | 100/108 (93%) | |

### Example 4

The NOV4 clone was analyzed, and the nucleotide and encoded polypeptide sequences are shown in Table 4A.

TABLE 4A
NOV4 Sequence Analysis

| | SEQ ID NO: 19 | 1076 bp | |
|---|---|---|---|
| NOV4a, | TCGTGGTGCTTGGGTGGTCGCCACCAAGAAGACTTTGGTGGGGTAGTCTCGGGGCAGCTCA | | |
| CG134430-01 | | | |
| DNA Sequence | GCGCCCCCCTGTCCCCCTTTCTGGCCTCGCTCCCACCTTGCACGTCGAGACTCGTAGCCCC | | |
| | CACCGTAGGGCGACCCTGCGGGTCGCCGCCGCGGCCGCCTCGGGCTCTGGGCCCAGCCGCA | | |
| | GCCTCTTCTACCGCGGCCGGTTGGGAGTCGCCGCCAGATGCAGCCTCCGGGCCCGCCCCCG | | |
| | GCCTATGCCCCCACTAACCCCGACTTCACCTTTCTCTCCTCACCACACCCCGAACATCTCA | | |
| | GTGGTTCAATAGCATCCCCAGATGTCAAATTAAATCTTGGTGGAGATTTTATCAAAGAATC | | |
| | TACACCTACTACATTTCTGACACAAAGAGGTTATGGCTCGCTTCTGCAAGTTGAAGATGAT | | |
| | GATCCTCAAGATAACAAGCCACTCTTGCAAGAATTCGACATTGATCTAAACGATATTTACT | | |
| | ACAAAATCCGATGTGTTTTGATGCCAATGCCATCACTTGGTTTTAATAGACAAGTGGTGAG | | |
| | AGACAATCCTGACTTTTGGCGTCCTCTCCCTGTTGTTCTTTTCTTTTCCATCATATCATTA | | |
| | TATGGACAGTTTAGGGTGGTCTCATGGATTATAACCATTTGGATATTTGGTTCACTAACAA | | |
| | TTTTCTTACTGGCCAGAGTTCTTGGTGGAGAAGTTGCATATGGCCAAGTCCTTGGAGTTAT | | |
| | AGGATATTCATTACTTCCTCTCATTGTAATACCCCCTCTACTTTTGCTGCTTGGATCATTT | | |
| | GAACTGGTCTCTACACTTATAAAAGTGAGAAGCACCAGAGGGACAGCACTTCTAGAAGTTA | | |
| | GAATAATATGAAGTAATCAGGAAATATCTATGCCTACAGAAGCAGCAACCGTAAGATAAAC | | |
| | ATTTGTTACACTTAAGAAATTGCTGACGTTAATACTTTCTTATAATGGATTATAATATTTG | | |
| | ACATTCATACTGTTGACCCTGGAATCTTTCACACAAAGCTTGGGCCTCAGGACCACCACGT | | |
| | AGAATTTTACAAGGCAATAAATGAAGGTCTTTTAAGATC | | |
| | ORF Start: ATG at 221 | | ORF Stop: TGA at 863 |
| | SEQ ID NO: 20 | 214 aa | MW at 23585.1kD |

| | |
|---|---|
| NOV4a | MQPPGPPPAYAPTNGDFTFVSSADAEDLSGSIASPDVKLNLGGDFIKESTATTFLRQRGYG |
| CG134430-01 | |
| Protein Sequence | WLLEVEDDDPEDNKPLLEELDIDLKDIYYKIRCVLMPMPSLCFNRQVVRDNPDFWGPLAVV |
| | LFFSMISLYGQFRVVSWIITIWIFGSLTIFLLARVLGGEVAYGQVLCVIGYSLLPLIVIAP |
| | VLLVVGSFEVVSTLIKVRSTRGTGLLEVRII |

One polymorphic variant of NOV4a has been identified and is shown in Table 41B. Further analysis of the NOV4a protein yielded the following properties shown in Table 4B.

TABLE 4B
Protein Sequence Properties NOV4a

| | |
|---|---|
| PSort analysis: | 0.6000 probability located in plasma |
| | membrane; 0.4000 probability located in |
| | Golgi body; 0.3000 probability located |
| | in endoplasmic reticulum (membrane); |
| | 0.1000 probability located in mitochondrial |
| | inner membrane |
| SignalP analysis: | No Known Signal Sequence Predicted |

A search of the NOV4a protein against the Geneseq database, a proprietary database that contains sequences published in patents and patent publication, yielded several homologous proteins shown in Table 4C.

TABLE 4C
Geneseq Results for NOV4a

| | | NOV4a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB89547 | Human polypeptide SEQ ID | 1 . . . 200 | 199/200 (99%) | e−113 |
| | NO 1923 - Homo sapiens, | 1 . . . 200 | 200/200 (99%) | |
| | 244 aa. [WO200190304-A2, | | | |
| | 29-NOV-2001] | | | |
| AAM40701 | Human polypeptide SEQ ID | 1 . . . 200 | 199/200 (99%) | e−113 |
| | NO 5632 - Homo sapiens, | 73 . . . 272 | 200/200 (99%) | |
| | 316 aa. [WO200153312-A1, | | | |
| | 26-JUL-2001] | | | |
| AAM38915 | Human polypeptide SEQ ID | 1 . . . 200 | 199/200 (99%) | e−113 |
| | NO 2060 - Homo sapiens, | 98 . . . 297 | 200/200 (99%) | |
| | 341 aa. [WO200153312-A1, | | | |
| | 26-JUL-2001] | | | |
| ABB11939 | Human secreted protein | 1 . . . 200 | 199/200 (99%) | e−113 |
| | homolog, SEQ ID NO: 2309 - | 31 . . . 230 | 200/200 (99%) | |
| | Homo sapiens, 274 aa. | | | |
| | [WO200157188-A2, 09- | | | |
| | AUG-2001] | | | |
| ABG02475 | Novel human diagnostic | 20 . . . 108 | 82/89 (92%) | 2e−42 |
| | protein #2466 - Homo | 209 . . . 297 | 85/89 (95%) | |
| | sapiens, 297 aa. | | | |
| | [WO200175067-A2, 11- | | | |
| | OCT-2001] | | | |

In a BLAST search of public sequence datbases, the NOV4a protein was found to have homology to the proteins shown in the BLASTP data in Table 4D.

TABLE 4D
Public BLASTP Results for NOV4a

| | | NOV4a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9BSR8 | Similar to RIKEN cDNA | 1 . . . 200 | 199/200 (99%) | e−112 |
| | 2310034L04 gene - Homo | 1 . . . 200 | 200/200 (99%) | |
| | sapiens (Human), 244 aa. | | | |
| Q99KZ9 | Hypothetical 32.8 kDa protein - | 26 . . . 200 | 169/177 (95%) | 2e−92 |
| | Mus musculus (Mouse), 289 | 69 . . . 245 | 174/177 (97%) | |
| | aa. | | | |
| Q9CYG0 | 2310034L04Rik protein - Mus | 1 . . . 138 | 135/140 (96%) | 2e−74 |
| | musculus (Mouse), 140 aa. | 1 . . . 140 | 137/140 (97%) | |
| Q9U1Y8 | Y60A3A.19 protein - | 29 . . . 195 | 89/168 (52%) | 7e−46 |
| | Caenorhabditis elegans, 255 | 40 . . . 206 | 118/168 (69%) | |
| | aa. | | | |
| Q9XTX4 | T08D2.6 protein - | 59 . . . 112 | 33/54 (61%) | 2e−11 |
| | Caenorhabditis elegans, 69 | 13 . . . 65 | 40/54 (73%) | |
| | aa. | | | |

PFam analysis predicts that the NOV4a protein contains the domains shown in Table 4E.

TABLE 4E
Domain Analysis of NOV4a

| | | | |
|---|---|---|---|
| Pfam | NOV4a | Identities/ | Expect |
| Domain | Match Region | Similarities | Value |
| | | for the Matched | |
| | | Region | |

### Example 5

The NOV5 clone was analyzed, and the nucleotide and encoded polypeptide sequences are shown in Table 5A.

TABLE 5A
NOV5 Sequence Analysis

| | SEQ ID NO: 21 | 1050 bp | |
|---|---|---|---|
| NOV5a, | TCCACGCAACGCTGCGGCTCCGCCCACGTCATGGCGCCCGAGGAGAACGCGGGGACAGAAC | | |
| CG137677-01 | | | |
| DNA Sequence | TCTGGCTCCAGGCTTTCGAGCGCCGCTTCCTGCCGGCGCCCTCACTGCGCTCCTTCCCCTG | | |
| | GCACAGCTTACAGGCAAAGTTAAGACACTCATCAGATTCTGAGCTGCTCCGGGATATTTTG | | |
| | CAGAACACTGTGAAGCATCCCGTGTGTGTGAAGCACCCGCCATCAGTCAAGTATGCCCGGT | | |
| | GCTTTCTCTCACAACTCATCAAAAAGGTCAGTGCTCTCCACACCGAGCCTTTGGACGAGCT | | |
| | GTACGAGGTGCTCGCGGAGACTCTGATGGCCAAGGACTCCACCCACGGCCACCGGAGCTAT | | |
| | TTGCTCCCCTCGGGAGCCTCGTTCACACTTTCCGAGATCACAGCCATCATCTCCCATGGTA | | |
| | CTACACGCCTGGTCACATGCGACGCCACCCTCTACCTTGCAGAATGGGCCATCGACAACCC | | |
| | AGCACCCTTCACTAACAGGGGTGTCCTAGAGCTTGGCAGTGGCGCTGGCCTCACACGCCTC | | |
| | GCCATCTGCAAGATGTGTCGCCCCCAGGCATACATCTTCAGCGACTGTCACAGCCGGGTCC | | |
| | TCGAGCAGCTCCGAGGGAATGTCCTTCTCAATGGCCTCTCATTAGAGGCAGACATCACTGC | | |
| | CAACTTACACGCCCCAAGGGTGACAGTGGCCCAGCTCGACTGGGACGTAGCGACAGTCCAT | | |
| | CAGCTCTCTGCCTTCCAGCCAGATATTGTCATTGCAGCAGACGTGCTGTATTGCCCAGAAG | | |
| | CCATCGTGTCACTGGTCGGGGTCCTGCGGAGGCTGGCTGCCTGCCGGGAGCACAACCACGC | | |
| | TCCTGAGGTCTACCTGGCCTTTACCGTCCGCAACCCAGAGACGTGCCAGCTCTTCACCACC | | |
| | GAGCTAGGTTGGACTCGGATCAGATGCGAAGTGGAAGCTCATCATGACCAGAAACTGTTTC | | |
| | CCTACAGAGAGCACTTCGAGATGGCAATGCTGAACCTCACACTGTAGGACTCACACACGAC | | |
| | TCCAACGGGCTTG | | |
| | ORF Start: ATG at 31 | | ORF Stop: TAG at 1021 |
| | SEQ ID NO: 22 | 330 aa | MW at 36826.8kD |

| | |
|---|---|
| NOV5a, | MAPEENAGTELWLQGFERRFLAARSLRSFPWQSLEAKLRDSSDSELLRDILQKTVKHPVCV |
| CG137677-01 | |
| Protein sequence | KHPPSVKYARCFLSELIKKVSAVHTEPLDELYEVLAETLMAKESTQCHRSYLLPSGGSFTL |
| | SEITAIISHGTTGLVTWDATLYLAEWAIENPAAFTNRGVLELGSGAGLTGLAICKMCRPQA |
| | YIFSDCHSRVLEQLRGNVLLNGLSLEADITANLDAPRVTVAQLDWDVATVHQLSAFQPDIV |
| | IAADVLYCPEAIVSLVGVLRRLAACREHKQAPEVYLAFTVRNPETCQLFTTELGWTGIRWE |
| | VEAHHDQKLFPYREHLEMAMLNLTL |

Further analysis of the NOV5a protein yielded the following properties shown in Table 5B.

TABLE 5B
Protein Sequence Properties NOV5a

| | |
|---|---|
| PSort analysis: | 0.7000 probability located in plasma |
| | membrane; 0.3902 probability located in |
| | microbody (peroxisome); 0.2000 probability |
| | located in endoplasmic reticulum |
| | (membrane); 0.1000 probability located |
| | in mitochondrial inner membrane |
| SignalP analysis: | No Known Signal Sequence Predicted |

A search of the NOV5a protein against the Geneseq database, a proprietary database that contains sequences published in patents and patent publication, yielded several homologous proteins shown in Table 5C.

TABLE 5C
Geneseq Results for NOV5a

| | | NOV5a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB36613 | Human FLEXHT-35 protein | 1 . . . 330 | 302/330 (91%) | e−174 |
| | sequence SEQ ID NO: 35 - | 1 . . . 330 | 312/330 (94%) | |
| | Homo sapiens, 330 aa. | | | |
| | [WO200070047-A2, 23- | | | |
| | NOV-2000] | | | |
| ABG13115 | Novel human diagnostic | 1 . . . 297 | 274/297 (92%) | e−158 |
| | protein #13106 - Homo | 23 . . . 319 | 284/297 (95%) | |
| | sapiens, 425 aa. | | | |
| | [WO200175067-A2, 11- | | | |
| | OCT-2001] | | | |
| ABG09575 | Novel human diagnostic | 1 . . . 330 | 259/379 (68%) | e−134 |
| | protein #9566 - Homo | 1 . . . 379 | 277/379 (72%) | |
| | sapiens, 379 aa. | | | |
| | [WO200175067-A2, 11- | | | |
| | OCT-2001] | | | |
| ABG13114 | Novel human diagnostic | 1 . . . 297 | 227/346 (65%) | e−113 |
| | protein #13105 - Homo | 1 . . . 346 | 245/346 (70%) | |
| | sapiens, 490 aa. | | | |
| | [WO200175067-A2, 11- | | | |
| | OCT-2001] | | | |
| AAU33207 | Novel human secreted protein | 33 . . . 297 | 209/266 (78%) | e−108 |
| | #3698 - Homo sapiens, 352 | 8 . . . 246 | 217/266 (81%) | |
| | aa. [WO200179449-A2, 25- | | | |
| | OCT-2001] | | | |

In a BLAST search of public sequence datbases, the NOV5a protein was found to have homology to the proteins shown in the BLASTP data in Table 5D.

TABLE 5D
Public BLASTP Results for NOV5a

| | | NOV5a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96G04 | Similar to RIKEN cDNA | 1 . . . 330 | 302/330 (91%) | e−174 |
| | 5730409G15 gene - Homo | 1 . . . 330 | 312/330 (94%) | |
| | sapiens (Human), 330 aa. | | | |
| Q96S85 | Hypothetical 33.0 kDa protein - | 1 . . . 330 | 272/330 (82%) | e−152 |
| | Homo sapiens (Human), 296 | 1 . . . 296 | 282/330 (85%) | |
| | aa. | | | |
| Q9CS89 | 5730409G15Rik protein - | 1 . . . 298 | 214/298 (71%) | e−117 |
| | Mus musculus (Mouse), 319 | 1 . . . 297 | 242/298 (80%) | |
| | aa (fragment). | | | |
| BAC05241 | CDNA FLJ40819 fis, clone | 1 . . . 159 | 113/159 (71%) | 1e−53 |
| | TRACH2010771 - Homo | 1 . . . 125 | 116/159 (72%) | |
| | sapiens (Human), 153 aa. | | | |
| Q9NVL1 | CDNA FLJ10661 fis, clone | 1 . . . 114 | 79/114 (69%) | 4e−33 |
| | NT2RP2006106 - Homo | 1 . . . 87 | 83/114 (72%) | |
| | sapiens (Human), 165 aa. | | | |

PFam analysis predicts that the NOV5a protein contains the domains shown in Table 5E.

TABLE 5E
Domain Analysis of NOV5a

| | | | |
|---|---|---|---|
| Pfam | NOV5a | Identities/ | Expect |
| Domain | Match Region | Similarities | Value |
| | | for the Matched | |
| | | Region | |

### Example 6

The NOV6 clone was analyzed, and the nucleotide and encoded polypeptide sequences are shown in Table 6A.

TABLE 6A
NOV6 Sequence Analysis

| | SEQ ID NO: 23 | 948 bp | |
|---|---|---|---|
| NOV6a, | TCCAGGCAACGCTCCGGCTCCGCCCACGTCATGGCGCCCGAGGAGAACGCGGGGACAGAAC | | |
| CG137697-01 | | | |
| DNA Sequence | TCTGGCTGCAGGGTTTCGAGCGCCGCTTCCTGGCGGCGCGCTCACTGCGCTCCTTCCCCTG | | |
| | GCAGAGCTTAGAGGCAAAGTTAAGAGACTCATCAGATTCTGAGCTGCTGCGGGATATTTTG | | |
| | CAGAAGACTGTGAAGCATCCCGTGTGTGTGAAGCACCCGCCATCAGTCAAGTATGCCCGGT | | |
| | GCTTTCTCTCAGAACTCATCAAAAAGCCCTCGGGAGGCTCGTTCACACTTTCCGAGATCAC | | |
| | AGCCATCATCTCCCATGGTACTACAGGCCTGGTCACATGGGACGCCACCCTCTACCTTGCA | | |
| | GAATGGGCCATCGAGAACCCAGCAGCCTTCACTAACAGGGGTGTCCTAGACCTTGGCAGTG | | |
| | GCGCTGGCCTCACAGGCCTGGCCATCTGCAAGATGTGTCGCCCCCAGGCATACATCTTCAG | | |
| | CGACTGTCACAGCCGGGTCCTCGAGCAGCTCCGAGGGAATGTCCTTCTCAATGGCCTCTCA | | |
| | TTAGAGGCAGACATCACTGCCAACTTAGACGCCCCAAGGGTGACAGTGGCCCAGCTGGACT | | |
| | GGGACGTAGCGACAGTCCATCAGCTCTCTGCCTTCCAGCCAGATATTGTCATTGCAGCAGA | | |
| | CGTGCTGTATTGCCCAGAAGCCATCGTGTCACTGGTCGGGGTCCTGCGGAGGCTCGCTGCC | | |
| | TGCCGGGAGCACAAGCAGGCTCCTGAGGTCTACCTGGCCTTTACCGTCCGCAACCCAGAGA | | |
| | CGTGCCAGCTGTTCACCACCGAGCTAGGTTGGACTGGGATCAGATGGGAAGTGGAAGCTCA | | |
| | TCATGACCAGAAACTGTTTCCCTACAGAGAGCACTTGGAGATGGCAATGCTGAACCTCACA | | |
| | CTGTAGGACTCACACACGACTCCAACGGGCTTG | | |
| | ORF Start: ATG at 31 | | ORF Stop: TAG at 919 |
| | SEQ ID NO: 24 | 296 aa | MW at 33013.5kD |

| | |
|---|---|
| NOV6a, | MAPEENAGTELWLQGFERRFLAARSLRSFPWQSLEAKLRDSSDSELLRDILQKTVKHPVCV |
| CG137697-01 | |
| Protein Sequence | KHPPSVKYARCFLSELIKKPSGGSFTLSEITAIISHGTTGLVTWDATLYLAEWAIENPAAP |
| | TNRGVLELGSGAGLTGLAICKMCRPQAYIFSDCHSRVLEQLRGNVLLNGLSLEADITANLD |
| | APRVTVAQLDWDVATVHQLSAFQPDIVIAADVLYCPEAIVSLVGVLRRLAACREHKQAPEV |
| | YLAFTVRNPETCQLFTTELGWTGIRWEVEAHHDQKLFPYREHLEMANLNLTL |

Further analysis of the NOV6a protein yielded the following properties shown in Table 6B.

TABLE 6B
Protein Sequence Properties NOV6a

| | |
|---|---|
| PSort analysis: | 0.7000 probability located in plasma |
| | membrane; 0.4382 probability located in |
| | microbody (peroxisome); 0.2000 probability |
| | located in endoplasmic reticulum |
| | (membrane); 0.1000 probability located |
| | in mitochondrial inner membrane |
| SignalP analysis: | No Known Signal Sequence Predicted |

A search of the NOV6a protein against the Geneseq database, a proprietary database that contains sequences published in patents and patent publication, yielded several homologous proteins shown in Table 6C.

TABLE 6C
Geneseq Results for NOV6a

| | | NOV6a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB36613 | Human FLEXHT-35 protein | 1 . . . 296 | 271/330 (82%) | e−151 |
| | sequence SEQ ID NO: 35 - | 1 . . . 330 | 281/330 (85%) | |
| | Homo sapiens, 330 aa. | | | |
| | [WO200070047-A2, 23-NOV-2000] | | | |
| ABG13115 | Novel human diagnostic | 1 . . . 263 | 243/297 (81%) | e−135 |
| | protein #13106 - Homo | 23 . . . 319 | 253/297 (84%) | |
| | sapiens, 425 aa. | | | |
| | [WO200175067-A2, 11- | | | |
| | OCT-2001] | | | |
| ABG09575 | Novel human diagnostic | 19 . . . 296 | 220/299 (73%) | e−114 |
| | protein #9566 - Homo | 89 . . . 379 | 233/299 (77%) | |
| | sapiens, 379 aa. | | | |
| | [WO200175067-A2, 11- | | | |
| | OCT-2001] | | | |
| ABG13114 | Novel human diagnostic | 19 . . . 263 | 188/266 (70%) | 7e−94 |
| | protein #13105 - Homo | 89 . . . 346 | 203/266 (75%) | |
| | sapiens, 490 aa. | | | |
| | [WO200175067-A2, 11- | | | |
| | OCT-2001] | | | |
| AAU33207 | Novel human secreted protein | 33 . . . 263 | 183/242 (75%) | 9e−92 |
| | #3698 - Homo sapiens, 352 | 8 . . . 246 | 194/242 (79%) | |
| | aa. [WO200179449-A2, 25- | | | |
| | OCT-2001] | | | |

In a BLAST search of public sequence datbases, the NOV6a protein was found to have homology to the proteins shown in the BLASTP data in Table 6D.

TABLE 6D
Public BLASTP Results for NOV6a

| | | NOV6a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96S85 | Hypothetical 33.0 kDa protein - | 1 . . . 296 | 272/296 (91%) | e−157 |
| | Homo sapiens (Human), 296 aa. | 1 . . . 296 | 282/296 (94%) | |
| Q96G04 | Similar to RIKEN cDNA | 1 . . . 296 | 271/330 (82%) | e−151 |
| | 5730409G15 gene - Homo | 1 . . . 330 | 281/330 (85%) | |
| | sapiens (Human), 330 aa. | | | |
| Q9CS89 | 5730409G15Rik protein - | 1 . . . 264 | 189/298 (63%) | 5e−98 |
| | Mus musculus (Mouse), 319 | 1 . . . 297 | 216/298 (72%) | |
| | aa (fragment). | | | |
| BAC05241 | CDNA FLJ40819 fis, clone | 1 . . . 125 | 113/125 (90%) | 6e−59 |
| | TRACH2010771 - Homo | 1 . . . 125 | 116/125 (92%) | |
| | sapiens (Human), 153 aa. | | | |
| AAH32519 | Similar to hypothetical | 1 . . . 70 | 51/70 (72%) | 7e−20 |
| | protein FLJ10661 - Homo sapiens | 1 . . . 66 | 58/70 (82%) | |
| | (Human), 131 aa. | | | |

PFam analysis predicts that the NOV6a protein contains the domains shown in Table 6E.

TABLE 6E
Domain Analysis of NOV6a

| | | | |
|---|---|---|---|
| Pfam | NOV6a | Identities/ | Expect |
| Domain | Match Region | Similarities | Value |
| | | for the Matched | |
| | | Region | |

### Example 7

The NOV7 clone was analyzed, and the nucleotide and encoded polypeptide sequences are shown in Table 7A.

TABLE 7A
NOV7 Sequence Analysis

| | SEQ ID NO: 25 | 1525 bp | |
|---|---|---|---|
| NOV7a, | GCGGCCGCCGCAGTGAGCAACGCGGCAACCCCACCCCGGCGGGCAGCCGGCGACGCCCGCA | | |
| CG137717-01 | | | |
| DNA Sequence | CTGAGAGGGCCGAGCCGCTCCTGCTCCCCCCCGGCAGAGGCCCGCGTCGCCCACGGGCCCG | | |
| | GGAGAGACCCGCTCCAGCCGGCCCCAGGATGTAGCCGATCGGCGGCAGCGCTCCTGCAGCC | | |
| | GGCCCGCTCATCATGAACAAGCACTCGCCCCGGGTCCCCCCGCTCAGCGCCTGCAACAGTC | | |
| | CGGTCCTGACCCTTACCAAAGTGGAAGGGGAGGAGCGCCCCCGGGACTCCCCGGGCCCGGC | | |
| | GGAGGCCCAGGCACCGGCCGGGGTGGAGGCCGGCGGGAGAGCGAGTCGCCGCTGCTGGACG | | |
| | TGCTCCCGGGCGCAACTCAAGAAGATCTTCTGGGGCGTGGCGGTCGTGCTGTGCGTGTGCT | | |
| | CCTCGTGGGCGGGCTCCACGCAGCTCGCCAAGCTGACCTTCAGGAAGTTCGACGCGCCCTT | | |
| | CACCCTCACGTGGTTTGCCACCAACTGGAACTTTTTATTCTTCCCGTTGTACTACGTGGGG | | |
| | CACGTCTGCAAGTCCACAGAGAAGCAGTCTGTGAAGCAGCGATACAGGGAATGCTCTCGAT | | |
| | TTTTTGGAGACAATGGCTTGACTTTGAAGGTGTTTTTTACCAAGGCAGCACCCTTTGGTGT | | |
| | TCTTTGGACACTCACAAACTACCTGTACTTACATGCAATAAAGAAAATAAACACTACGGAT | | |
| | GTCTCCGTGTTGTTCTGCTGCAACAAAGCTTTTGTGTTCTTGCTCTCATGGATCCTTCTCA | | |
| | GGGACAGATTCATGGGAGTGATTGTGGCCGCCATCCTCGCCATCGCTGGCATTGTGATGAT | | |
| | GACCTACGCTGATGGCTTCCACAGCCACTCCGTCATCGGCATCGCACTGGTGGTGGCCTCA | | |
| | GCATCGGTTTTGTTCAAGCTCCTCCTGGGCAGTGCTAAGTTTGGAGAAGCCGCCTTATTTT | | |
| | TGTCCATCTTGGGTGTGTTTAACATCCTCTTCATCACCTGCATTCCTATTATCCTCTACTT | | |
| | TACCAAAGTCGAATACTCGAGCTCTTTTGATGACATTCCATGGGGAAACCTTTCTGGATTT | | |
| | TCACTTCTTTTATTGGCATTCAATATTGTATTAAATTTTGGAATTCCCGTTACATATCCCA | | |
| | CTCTGATGTCTCTTGGAATCGTCCTCACCATACCTGTGAATGCAGTCATTGATCACTACAC | | |
| | CAGTCAGATCGTCTTCAATGCCGTCCGGGTCATCGCCATCATCATCATCGGCCTGCGTTTT | | |
| | CTCCTCCTGCTCCTGCCAGAGGAGTGGCATGTCTCCTTGATCAAGCTCCTCACCCGACTCA | | |
| | AAGTGAGGAAGAAGGAGCAGCCTGCAGACGCCGCTGCCGACCTGAGCTCAGCACCTCACAG | | |
| | CAAGAACACAAGAGCCCGGCCTTCCTTCGCCCGCTAACACCACTCCTCTAGAACTCGGTGG | | |
| | TAATGACTCGGAGGTCTATTCCTCCCGGGACCAACCTCAGTTGGGTAAGGTGTACATACCT | | |
| | ORF Start: ATG at 196 | | ORF Stop: TAA at 1438 |
| | SEQ ID NO: 26 | 414 aa | MW at 45936.7kD |

| | |
|---|---|
| NOV7a | MKKHSARVAPLSACNSPVLTLTKVEGEERPRDSPGPAEAQAPAGVEAGGRASRRCWTCSRA |
| CG137717-01 | |
| Protein Sequence | QLKKIFWGVAVVLCVCSSWAGSTQLAKLTFRKFDAPFTLTWFATNWNFLFFPLYYVGHVCK |
| | STEKQSVKQRYRECCRFFGDNGLTLKVFFTKAAPFGVLWTLTNYLYLHAIKKINTTDVSVL |
| | FCCNKAFVFLLSWIVLRDRFMGVIVAAILAIAGIVMMTYADGFHSHSVIGIALVVASASVL |
| | FKLLLCSAKFGEAALFLSILGVFNILFITCIPIILYFTKVEYWSSFDDIPWCNLCGFSVLL |
| | LAFNIVLNFGIAVTYPTLMSLGIVLSIPVNAVIDHYTSQIVFNOVRVIAIIIIGLGFLLLL |
| | LPEEWDVWLIKLLTRLKVRKKEEPAEGAADLSSGPQSKNRRARPSFAR |

Further analysis of the NOV7a protein yielded the following properties shown in Table 7B.

TABLE 7B
Protein Sequence Properties NOV7a

| | |
|---|---|
| PSort analysis: | 0.6000 probability located in plasma |
| | membrane; 0.4663 probability located in |
| | mitochondrial inner membrane; |
| | 0.4000 probability located in Golgi body; |
| | 0.3000 probability located in endoplasmic |
| | reticulum (membrane) |
| SignalP analysis: | No Known Signal Sequence Predicted |

A search of the NOV7a protein against the Geneseq database, a proprietary database that contains sequences published in patents and patent publication, yielded several homologous proteins shown in Table 7C.

TABLE 7C
Geneseq Results for NOV7a

| | | NOV7a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABG16671 | Novel human diagnostic | 5 . . . 284 | 160/329 (48%) | 2e−80 |
| | protein #16662 - Homo | 168 . . . 492 | 208/329 (62%) | |
| | sapiens, 531 aa. | | | |
| | [WO200175067-A2, 11- | | | |
| | OCT-2001] | | | |
| ABB89266 | Human polypeptide SEQ ID | 1 . . . 134 | 134/134 (100%) | 1e−76 |
| | NO 1642 - Homo sapiens, | 1 . . . 134 | 134/134 (100%) | |
| | 134 aa. [WO200190304-A2, | | | |
| | 29-NOV-2001] | | | |
| AAM36449 | Peptide #10486 encoded by | 338 . . . 414 | 77/77 (100%) | 5e−37 |
| | probe for measuring | 1 . . . 77 | 77/77 (100%) | |
| | placental gene expression - | | | |
| | Homo sapiens, 77 aa. | | | |
| | [WO200157272-A2, 09- | | | |
| | AUG-2001] | | | |
| AAM76340 | Human bone marrow | 338 . . . 414 | 77/77 (100%) | 5e−37 |
| | expressed probe encoded | 1 . . . 77 | 77/77 (100%) | |
| | protein SEQ ID NO: 36646 - | | | |
| | Homo sapiens, 77 aa. | | | |
| | [WO200157276-A2, 09- | | | |
| | AUG-2001] | | | |
| AAM63526 | Human brain expressed | 338 . . . 414 | 77/77 (100%) | 5e−37 |
| | single exon probe encoded | 1 . . . 77 | 77/77 (100%) | |
| | protein SEQ ID NO: 35631 - | | | |
| | Homo sapiens, 77 aa. | | | |
| | [WO200157275-A2, 09- | | | |
| | AUG-2001] | | | |

In a BLAST search of public sequence datbases, the NOV7a protein was found to have homology to the proteins shown in the BLASTP data in Table 7D.

TABLE 7D
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAC04479 | CDNA FLJ37712 fis, clone | 27 . . . 414 | 387/395 (97%) | 0.0 |
| | BRHIP2018369 - Homo sapiens | 96 . . . 490 | 387/395 (97%) | |
| | (Human), 490 aa. | | | |
| Q9JJG8 | Brain cDNA, clone MNCb- | 114 . . . 406 | 179/300 (59%) | 1e−99 |
| | 0335 - Mus musculus | 26 . . . 325 | 227/300 (75%) | |
| | (Mouse), 335 aa. | | | |
| Q8T0 m8 | GH20388p - Drosophila | 94 . . . 379 | 102/295 (34%) | 7e−46 |
| | melanogaster (Fruit fly), 578 | 245 . . . 536 | 165/295 (55%) | |
| | aa. | | | |
| Q95XC7 | Hypothetical 37.3 kDa | 66 . . . 368 | 110/320 (34%) | 5e−39 |
| | protein - Caenorhabditis | 16 . . . 326 | 170/320 (52%) | |
| | elegans, 339 aa. | | | |
| Q9VDJ2 | CG15688 protein - | 94 . . . 211 | 47/119 (39%) | 2e−17 |
| | Drosophila melanogaster | 245 . . . 361 | 70/119 (58%) | |
| | (Fruit fly), 365 aa. | | | |

PFam analysis predicts that the NOV7a protein contains the domains shown in Table 7E.

TABLE 7E
Domain Analysis of NOV7a

| | | Identities/ | |
|---|---|---|---|
| Pfam | NOV7a | Similarities for | Expect |
| Domain | Match Region | the Matched Region | Value |
| DUF6 | 78 . . . 222 | 24/147 (16%) | 0.053 |
| | | 99/147 (67%) | |

### Example 8

The NOV8 clone was analyzed, and the nucleotide and encoded polypeptide sequences are shown in Table 8A.

TABLE 8A
NOV8 Sequence Analysis

| | SEQ ID NO: 27 | 898 bp | |
|---|---|---|---|
| NOV8a, | TAAGCACCAGGAGTCCATGAAGAAGATGGCTCCTGCCATGGAATCCCCTACTCTACTGTGT | | |
| CG137793-01 | | | |
| DNA Sequence | GTAGCCTTACTGTTCTTCGCTCCAGATGGCGTGTTAGCAGTCCCTCAGAAACCTAAGGTCT | | |
| | CCTTGAACCCTCCATGGAATAGAATATTTAAAGGAGAGAATGTGACTCTTACATGTAATGG | | |
| | GAACAATTTCTTTGAAGTCAGTTCCACCAAATGGTTCCACAATGGCAGCCTTTCAGAAGAG | | |
| | ACAAATTCAAGTTTGAATATTGTGAATGCCAAATTTGAAGACAGTGGAGAATACAAATGTC | | |
| | AGCACCAACAAGTTAATGAGAGTGAACCTGTGTACCTGGAACTCTTCAGTGACTGCCTGCT | | |
| | CCTTCAGGCCTCTGCTGAGGTGGTGATGGAGGGCCAGCCCCTCTTCCTCAGGTGCCATGGT | | |
| | TGGAGGAACTGGGATGTGTACAAGGTGATCTATTATAAGGATGGTGAAGCTCTCAAGTACT | | |
| | GGTATGAGAACCACAACATCTCCATTACAAATGCCACAGTTGAAGACAGTGGAACCTACTA | | |
| | CTGTACGGGCAAAGTGTGGCAGCTGGACTATGAGTCTGAGCCCCTCAACATTACTGTAATA | | |
| | AAAGCTCCGCGTGAGAAGTACTGGCTACAATTTTTTATCCCATTGTTGGTGGTGATTCTGT | | |
| | TTGCTGTCGACACAGGATTATTTATCTCAACCCAGCAGCAGGTCACATTTCTCTTGAACAT | | |
| | TAACAGAACCACGAAAGGCTTCAGACTTCTGAACCCACATCCTAAGCCAAACCCCAAAAAC | | |
| | AACTGATATAATTACTCAAGAAATATTTGCAACATTAGTTTTTTTCCACCATCAGCAATTG | | |
| | CTACTCAATTGTCAAACACACCTTGCAATAAAGGGCGATTCCAG | | |
| | ORF Start: ATG at 26 | | ORF Stop: TGA at 797 |
| | SEQ ID NO: 28 | 257 aa | MW at 29595.6kD |
| NOV8a | MAPAMESPTLLCVALLFFAPDGVLAVPQKPKVSLNPPWNRIFKGENVTLTCNGNNFFEVSS | | |
| CG137793-01 | | | |
| Protein Sequence | TKWFHNGSLSEETNSSLNIVNAKFEDSGEYKCQHQQVNESEPVYLEVFSDWLLLQASAEVV | | |
| | MEGQPLFLRCHGWRNWDVYKVTYYKDGEALKYWYENHNISITNATVEDSGTYYCTGKVWQL | | |
| | DYESEPLNITVIKAPREKYWLQFFIPLLVVILFAVDTGLFISTQQQVTFLLKIKRTRKGFR | | |
| | LLNPHPKPNPKNN | | |
| | SEQ ID NO: 29 | 757 bp | |
| NOV8b, | TAACCACCAGGAGTCCATGAAGAAGATGGCTCCTGCCATGCAATCCCCTACTCTACTGTGT | | |
| CG137793-02 | | | |
| DNA Sequence | GTAGCCTTACTCTTCTTCGCTCCAGATGGCGTGTTAGCAGTCCCTCAGAAACCTAAGGTCT | | |
| | CCTTGAACCCTCCATGGAATAGAATATTTAAAGGAGAGAATGTGACTCTTACATGTAATGG | | |
| | GAACAATTTCTTTGAAGTCACTTCCACCAAATGGTTCCACAATGGCAGCCTTTCAGAAGAC | | |
| | ACAAATTCAAGTTTGAATATTGTGAATGCCAAATTTCAAGACAGTGGAGAATACAAATGCC | | |
| | ATGGTTGGAGGAACTGGGATGTGTACAAGGTGATCTATTATAAGGATGGTGAAGCTCTCAA | | |
| | GTACTGGTATGAGAACCACAACATCTCCATTACAAATGCCACAGTTGAAGACAGTCGAACC | | |
| | TACTACTGTACGGGCAAAGTGTGGCAGCTGGACTATGAGTCTCAGCCCCTCAACATTACTG | | |
| | TAATAAAAGCTCCGCGTGAGAAGTACTCGCTACAATTTTTTATCCCATTGTTGCTGGTGAT | | |
| | TCTGTTTGCTGTGGACACAGCATTATTTATCTCAACTCAGCAGCAGGTCACATTTCTCTTC | | |
| | AAGATTAAGAGAACCAGGAAAGGCTTCAGACTTCTGAACCCACATCCTAAGCCAAACCCCA | | |
| | AAAACAACTGATATAATTACTCAACAAATATTTGCAACATTAGTTTTTTTCCAGCATCAGC | | |
| | AATTGCTACTCAATTGTCAAACACA | | |
| | ORF Start: ATG at 26 | | ORF Stop: TGA at 680 |
| | SEQ ID NO: 30 | 218 aa | MW at 25079.5kD |

| | |
|---|---|
| NOV8b, | MAPANESPTLLCVALLFFAPDGVLAVPQKPKVSLNPPWNRIFKGENVTLTCNQNNFFEVSS |
| CG137793-02 | |
| Protein Sequence | TKWFHNCSLSEETNSSLNIVNAKFEDSGEYKCHGWRNWDVYKVIYYKDGEALKYWYENHNI |
| | SITNATVEDSGTYYCTGKVWQLDYESEPLNITVIKAPREKYWLQFFIPLLVVILFAVDTGL |
| | FISTQQQVTFLLKIKRTRKGFRLLNPHPKPNPKNN |

Sequence comparison of the above protein sequences yields the following sequence relationships shown in Table 8B.

TABLE 8B
Comparison of NOV8a against NOV8b.

| Protein | NOV8a Residues/ | Identities/Similarities |
|---|---|---|
| Sequence | Match Residues | for the Matched Region |
| NOV8b | 1 . . . 246 | 207/246 (84%) |
| | 1 . . . 207 | 207/246 (84%) |

Twenty polymorphic variants of NOV8b have been identified and are shown in Table 41C.

Further analysis of the NOV8a protein yielded the following properties shown in Table 8C.

TABLE 8C
Protein Sequence Properties NOV8a

| | |
|---|---|
| PSort analysis: | 0.4600 probability located in plasma |
| | membrane; 0.1594 probability located in |
| | microbody (peroxisome); 0.1000 |
| | probability located in endoplasmic reticulum |
| | (membrane); 0.1000 probability located |
| | in endoplasmic reticulum (lumen) |
| SignalP analysis: | Cleavage site between residues 26 and 27 |

A search of the NOV8a protein against the Geneseq database, a proprietary database that contains sequences published in patents and patent publication, yielded several homologous proteins shown in Table 8D.

TABLE 8D
Geneseq Results for NOV8a

| | | NOV8a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAB31584 | Amino acid sequence of a | 1 . . . 257 | 257/257 (100%) | e−155 |
| | human Fc epsilon receptor | 1 . . . 257 | 257/257 (100%) | |
| | alpha-chain - Homo sapiens, | | | |
| | 257 aa. [WO200104310-A1, | | | |
| | 18-JAN-2001] | | | |
| AAB74667 | Human immunoglobulin E | 1 . . . 257 | 257/257 (100%) | e−155 |
| | receptor I alpha subunit | 1 . . . 257 | 257/257 (100%) | |
| | protein - Homo sapiens, 257 | | | |
| | aa. [WO200111010-A2, 15- | | | |
| | FEB-2001] | | | |
| AAY96230 | Human Fc receptor, | 1 . . . 257 | 257/257 (100%) | e−155 |
| | FcepsilonRIa - Homo | 4 . . . 260 | 257/257 (100%) | |
| | sapiens, 260 aa. | | | |
| | [EP1006183-A1, 07-JUN- | | | |
| | 2000] | | | |
| AAW61190 | The alpha chain of a Fc | 1 . . . 257 | 257/257 (100%) | e−155 |
| | epsilon receptor - Homo | 1 . . . 257 | 257/257 (100%) | |
| | sapiens, 257 aa. | | | |
| | [WO9823964-A1, 04-JUN- | | | |
| | 1998] | | | |
| AAW24066 | Alpha subunit of human high | 1 . . . 257 | 257/257 (100%) | e−155 |
| | affinity receptor for IgE | 1 . . . 257 | 257/257 (100%) | |
| | (human FcERI) - Homo | | | |
| | sapiens, 257 aa. | | | |
| | [US5639660-A, 17-JUN- | | | |
| | 1997] | | | |

In a BLAST search of public sequence datbases, the NOV8a protein was found to have homology to the proteins shown in the BLASTP data in Table 8E.

TABLE 8E
Public BLASTP Results for NOV8a

| | | NOV8a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P12319 | High affinity | 1 . . . 257 | 257/257 (100%) | e−154 |
| | immunoglobulin epsilon | 1 . . . 257 | 257/257 (100%) | |
| | receptor alpha-subunit | | | |
| | precursor (FcERI) (IgE Fc | | | |
| | receptor, alpha-subunit) (Fc- | | | |
| | epsilon RI-alpha) - Homo | | | |
| | sapiens (Human), 257 aa. | | | |
| AAH15195 | Fc IgE, high affinity I, | 1 . . . 257 | 256/257 (99%) | e−154 |
| | receptor for, alpha | 1 . . . 257 | 256/257 (99%) | |
| | polypeptide - Homo sapiens | | | |
| | (Human), 257 aa. | | | |
| CAC28464 | Sequence 4 from Patent | 26 . . . 257 | 232/232 (100%) | e−139 |
| | WO0104310 - Homo sapiens | 1 . . . 232 | 232/232 (100%) | |
| | (Human), 232 aa (fragment). | | | |
| CAC28471 | Sequence 26 from Patent | 1 . . . 197 | 197/197 (100%) | e−117 |
| | WO0104310 - Cloning vector | 1 . . . 197 | 197/197 (100%) | |
| | pINT1, 660 aa. | | | |
| CAC28468 | Sequence 17 from Patent | 1 . . . 197 | 197/197 (100%) | e−117 |
| | WO0104310 - Cloning vector | 1 . . . 197 | 197/197 (100%) | |
| | pINT1, 756 aa (fragment). | | | |

PFam analysis predicts that the NOV8a protein contains the domains shown in Table 8F.

TABLE 8F
Domain Analysis of NOV8a

| | | Identities/ | |
|---|---|---|---|
| | | Similarities | |
| Pfam | NOV8a | for the Matched | Expect |
| Domain | Match Region | Region | Value |
| ig | 44 . . . 95 | 19/54 (35%) | 1.4e−10 |
| | | 37/54 (69%) | |
| ig | 125 . . . 178 | 14/56 (25%) | 0.00018 |
| | | 37/56 (66%) | |

### Example 9

The NOV9 clone was analyzed, and the nucleotide and encoded polypeptide sequences are shown in Table 9A.

TABLE 9A
NOV9 Sequence Analysis

| | SEQ ID NO: 31 | 4330 bp | |
|---|---|---|---|
| NOV9a, | TCTAGGAGCCAGCCCCACCCTTAGAAAAGATGTTTTCCATGAGGATCGTCTGCCTGGTCCT | | |
| CG137873-01 | | | |
| DNA Sequence | AAGTGTGGTGGGCACAGCATGGACTGCAGATAGTGGTGAAGGTGACTTTCTAGCTGAAGGA | | |
| | GGAGGCGTGCGTGGCCCAAGGGTTGTGGAAAGACATCAATCTGCCTGCAAAGATTCAGACT | | |
| | GGCCCTTCTGCTCTGATGAAGACTGGAACTACAAATGCCCTTCTGGCTGCAGGATGAAAGG | | |
| | GTTGATTGATGAAGTCAATCAAGATTTTACAAACAGAATAAATAAGCTCAAAAATTCACTA | | |
| | TTTGAATATCAGAAGAACAATAAGGATTCTCATTCGTTGACCACTAATATAATGGAAATTT | | |
| | TGAGAGGCGATTTTTCCTCAGCCAATAACCGTGATAATACCTACAACCGAGTGTCAGAGGA | | |
| | TCTGAGAAGCAGAATTGAAGTCCTGAAGCGCAAAGTCATAGAAAAAGTACAGCATATCCAG | | |
| | CTTCTGCAGAAAAATGTTAGAGCTCAGTTGGTTGATATGAAACGACTGGAGGTGGACATTG | | |
| | ATATTAAGATCCGATCTTGTCGAGGGTCATGCAGTAGGGCTTTAGCTCGTGAAGTAGATCT | | |
| | GAAGGACTATGAAGATCAGCAGAAGCAACTTGAACAGGTCATTGCCAAAGACTTACTTCCC | | |
| | TCTAGAGATAGGCAACACTTACCACTGAThAAAATCAAACCAGTTCGAGACTTGGTTCCCG | | |
| | GAAATTTTAAGAGCCAGCTTCAGAAGGTACCCCCAGAGTGGAAGGCATTAACAGACATGCC | | |
| | GCAGATGAGAATGGAGTTAGAGACACCTGGTGGAAATGAGATTACTCGAGGAGGCTCCACC | | |
| | TCTAGAGATAGGCAACACTTACCACTGATAAAAATGAAACCAGTTCCAGACTTGGTTCCCG | | |
| | ACTCTGGGAGCTCTGGACCTGGIAGTACTGGAAACCGAAACCCTGGGAGCTCTGGGACTGG | | |
| | AGGGACTGCAACCTGGAAACCTGGGAGCTCTGGACCTGGAAGTACTGGAAGCTGGAACTCT | | |
| | GGGAGCTCTGGAACTGGAAGTACTGGAAACCAAAACCCTGGGAGCCCTAGACCTGGTAGTA | | |
| | CCGGAACCTGGAATCCTGGCAGCTCTGAACGCGGAAGTGCTGGGCACTGGACCTCTGAGAG | | |
| | CTCTGTATCTGGTAGTACTGGACAATGGCACTCTGAATCTGGAAGTTTTAGGCCAGATAGC | | |
| | CCAGGCTCTGGGAACGCGAGGCCTAACAACCCAGACTGGGGCACATTTGAAGAGGTGTCAG | | |
| | GAAATGTAAGTCCAGGGACAAGGAGAGAGTACCACACAGAAAAACTGGTGACTTCTAAAGG | | |
| | AGATAAAGAGCTCAGGACTGGTAAAGAGAAGGTCACCTCTGGTAGCACAACCACCACGCGT | | |
| | CGTTCATGCTCTAAAACCGTTACTAAGACTGTTATTGGTCCTGATGGTCACAAAGAAGTTA | | |
| | CCAAAGAAGTGGTGACCTCCGAAGATGGTTCTGACTGTCCCGAGGCAATGGATTTAGGCAC | | |
| | ATTGTCTGGCATAGGTACTCTGGATGGGTTCCGCCATAGGCACCCTGATGAAGCTGCCTTC | | |
| | TTCGACACTGCCTCAACTGGAAAAACATTCCCAGGTTTCTTCTCACCTATGTTAGGAGAGT | | |
| | TTGTCAGTGAGACTGAGTCTAGGGGCTCAGAATCTGGCATCTTCACAAATACAAAGGAATC | | |
| | CAGTTCTCATCACCCTGGGATAGCTGAATTCCCTTCCCGTGGTAAATCTTCAAGTTACAGC | | |
| | AAACAATTTACTAGTAGCACGAGTTACAACAGAGGAGACTCCACATTTGAAAGCAAGAGCT | | |
| | ATAAAATGGCAGATGAGGCCGGAAGTGAAGCCGATCATGAAGGAACACATAGCACCAAGAG | | |
| | AGGCCATGCTAAATCTCGCCCTGTCAGAGGTATCCACACTTCTCCTTTGGGGAAGCCTTCC | | |
| | CTGTCCCCCTAGACTAAGTTAAATATTTCTGCACAGTGTTCCCATGGCCCCTTGCATTTCC | | |
| | TTCTTAACTCTCTGTTACACGTCATTGAAACTACACTTTTTTGGTCTGTTTTTGTGCTAGA | | |
| | CTGTAAGTTCCTTGGGGGCAGGGCCTTTGTCTGTCTCATCTCTGTATTCCCAAATGCCTAA | | |
| | CAGTACAGAGCCATGACTCAATAAATACATGTTAAATGGATGAATGAATTCCTCTGAAACT | | |
| | CTATTTGAGCTTATTTAGTCAAATTCTTTCACTATTCAAAGTGTGTGCTATTAGAATTGTC | | |
| | ACCCAACTGATTAATCACATTTTTAGTATGTGTCTCAGTTGACATTTAGGTCAGGCTAAAT | | |
| | ACAAGTTGTGTTAGTATTAAGTGATGCTTAGCTACCTGTACTGGTTACTTGCTATTAGTTT | | |
| | GTGCAAGTAAAATTCCAAATACATTTGAGGAAAATCCCCCTTTGCAATTTGTAGGTATAAT | | |
| | AACCGCTTATTTGCATAAGTTCTATCCCACTGTAAGTGCATCCTTTCCCTATGGAGGGAAG | | |
| | GAAAGGAGGAAGAAAGAAAGGAAGGGAAAGAAACAGTATTTGCCTTATTTAATCTGAGCCG | | |
| | TGCCTATCTTTGTAAAGTTAAATGAGAATAACTTCTTCCAACCAGCTTAATTTTTTTTTTA | | |
| | GACTGTGATGATGTCCTCCAAACACATCCTTCAGGTACCCAAAGTGGCATTTTCAATATCA | | |
| | AGCTACCGGGATCCAGTAAGATTTTTTCTGTTTATTGCGATCAAGAGACCAGTTTGGGAGG | | |
| | ATGGCTTTTGATCCAGCAAAGAATGGATGGATCACTGAATTTTAACCGGACCTGGCAAGAC | | |
| | TACAAGAGAGGTTTCGGCAGCCTGAATGACGAGGGGGAAGGAGAATTCTGGCTAGGCAATG | | |
| | ACTACCTCCACTTACTAACCCAAAGGGGCTCTGTTCTTAGGGTTGAATTAGAGGACTGGGC | | |
| | TGGGAATGAAGCTTATGCAGAATATCACTTCCGGGTAGGCTCTGAGGCTGAAGGCTATGCC | | |
| | CTCCAAGTCTCCTCCTATGAAGGCACTGCGGGTGATGCTCTGATTGAGGGTTCCGTAGAGG | | |
| | AAGGGGCAGAGTACACCTCTCACAACAACATGCAGTTCAGCACCTTTGACAGGGATGCAGA | | |
| | CCAGTGGGAAGAGAACTGTGCAGAAGTCTATGGGGGAGGCTGGTGGTATAATAACTGCCAA | | |
| | GCAGCCAATCTCAATGGAATCTACTACCCTGGGGGCTCCTATGACCCAAGGAATAACAGTC | | |
| | CTTATGAGATTGAGAATGGAGTGGTCTGGGTTTCCTTTAGAGGGGCAGATTATTCCCTCAG | | |
| | GGCTGTTCGCATGAAAATTAGGCCCCTTGTGACCCAATAGGCTGAAGAAGTGGGAATGGGA | | |
| | GCACTCTGTCTTCTTTGCTAGAGAAGTGGAGAGAAAATACAAAAGGTAAAGCAGTTGAGAT | | |
| | TCTCTACAACCTAAAAAATTCCTAGGTGCTATTTTCTTATCCTTTGTACTGTAGCTAAATG | | |
| | TACCTGAGACATATTAGTCTTTGAAAAATAAAGTTATGTAAGGTTTTTTTTATCTTTAAAT | | |
| | AGCTCTGTGGGTTTTAACATTTTTGTAAAGATATACCAAGGGCCATTCAGTACATCAGGAA | | |
| | AGTGGCAGACAGAAGCTTCTCTCTGCAACCTTGAAGACTATTGGTTTGAGAACTTCTCTTC | | |
| | CCATACCACCCAAAATCATAATGCCATTGGAAAGCAAAAAGTTGTTTTATCCATTTGATTT | | |
| | GAATTGTTTTAAGCCAATATTTTAAGGTAAAACTCACTGAATCTAACCATAGCTGACCTTT | | |
| | GTAGTAGAATTTACAACTTATAATTACAATGCACAATTTATAATTACAATATGTATTTATG | | |
| | TCTTTTGCTATGGAGCAAATCCAGGAAGGCAAGAGAAACATTCTTTCCTAAATATAAATGA | | |
| | AAATCTATCCTTTAAACTCTTCCACTAGACGTTGTAATGCACACTTATTTTTTTCCCAAGG | | |
| | AGTAACCAATTTCTTTCTAAAACACATTTAAAATTTTAAAACTATTTATGAATATTAAAAA | | |
| | AAGACATAATTCACACATTAATAAACAATCTCCCAAGTATTGATTTAACTTCATTTTTCTA | | |
| | ATAATCATAAACTATATTCTGTGACATGCTAATTATTATTAAATGTAAGTCGTTAGTTCGA | | |
| | AAGCCTCTCACTAAGTATGATCTATGCTATATTCAAAATTCAACCCATTTACTTTGGTCAA | | |
| | TATTTGATCTAAGTTGCATCTTTAATCCTGGTGGTCTTGCCTTCTGATTTTTAATTTGTAT | | |
| | CCTTTTCTATTAAGATATATTTGTCATTTTCTCTTGAATATGTATTAAAATATCCCAAGC | | |
| | ORF Start: ATG at 30 | | ORF Stop: TAG at 1962 |
| | SEQ ID NO: 32 | 644 aa | MW at 69756.0kD |
| NOV9a, | MFSMRIVCLVLSVVGTAWTADSGEGDFLAEGGGVRGPRVVERHQSACKDSDWPFCSDEDWN | | |
| CG137873-01 | | | |
| Protein Sequence | YKCPSGCRMKGLIDEVNQDFTNRINKLKNSLFEYQKNNKDSHSLTTNIMEILRGDFSSANN | | |
| | RDNTYNRVSEDLRSRIEVLKRKVIEKVQHIQLLQKNVRAQLVDMKRLEVDIDIKIRSCRGS | | |
| | CSRALAREVDLKDYEDQQKQLEQVIAKDLLPSRDRQHLPLIKMKPVPDLVPGNFKSQLQKV | | |
| | PPEWKALTDMPQMRMELERPGGNEITRGGSTSYCTGSETESPRNPSSAGSWNSGSSGPGST | | |
| | GNRNPGSSGTGGTATWKPGSSGPGSTGSWNSGSSGTGSTGNQNPGSPRPGSTGTWNPGSSE | | |
| | RGSACHWTSESSVSGSTCQWISESGSFRPDSPGSGNARPNNPDWGTFEEVSGNVSPCTRRE | | |
| | YGTEKLVTSKGDKELRTGKEKVTSGSTTTTRRSCSKTVTKTVIGPDGHKEVTKEVVTSEDG | | |
| | SDCPEAMDLCTLSGICTLDCFRHRHPDEAAFFDTASTGKTFPGFFSPMLCEFVSETESRGS | | |
| | ESGIFTNTKESSSHHPGIAEFPSRGKSSSYSKQFTSSTSYNRGDSTFESKSYKMADEAGSE | | |
| | ADHEGTHSTKRGHAKSRPVRCIHTSPLGKPSLSP | | |
| | SEQ ID NO: 33 | 1515 bp | |
| NOV9b, | AATCCTTTCTTTCAGCTGGAGTGTCCTCAGGAGCCAGCCCCACCCTTAGAAAAGATGTTTT | | |
| CG137873-03 | | | |
| DNA Sequence | CCATGAGGATCGTCTGCCTGCTCCTAAGTGTGGTGCGCACAGCATGGACTCCAGATAGTGG | | |
| | TGAAGGTGACTTTCTAGCTGAAGGAGGAGGCGTGCGTGGCCCAAGGGTTGTGGAAAGACAT | | |
| | CAATCTGCCTGCAAAGATTCAGACTGGCCCTTCTGCTCTGATGAAGACTGGAACTACAAAT | | |
| | GCCCTTCTGGCTGCAGGATGAAAGGGTTGATTGATGAAGTCAATCAAGATTTTACAAACAG | | |
| | AATAAATAAGCTCAAAAATTCACTATTTGAATATCAGAAGAACAATAAGGATTCTCATTCG | | |
| | TTGACCACTAATATAATGGAAATTTTGAGAGGCGATTTTTCCTCAGCCAATAACCGTGATA | | |
| | ATACCTACAACCGAGTGTCAGAGGATCTGAGAAGCAGAATTGAAGTCCTGAAGCGCAAAGT | | |
| | CATAGAAAAAGTACAGCATATCCAGCTTCTGCAAAAAAATGTTAGAGCTCAGTTGGTTGAT | | |
| | ATGAAACGACTGGAGGTGGACATTGATATTAAGATCCGATCTTGTCGAGGGTCATGCAGTA | | |
| | GGGCTTTAGCTCGTGAAGTAGATCTGAAGGACTATGAAGATCAGCAGAAGCAACTTGAACA | | |
| | GGTCATTGCCAAAGACTTACTTCCCTCTAGAGATAGGCAACACTTACCACTGATCAAAATG | | |
| | AAACCAGTTCCAGACTTGGTTCCCGGAAATTTTAAGAGCCAGCTTCAGAAGGTACCCCCAG | | |
| | AGTGGAAGGCATTAACAGACATGCCGCAGATGAGAATGGAGTTAGAGAGACCTGGTGGAAA | | |
| | TGAGATTACTCGAGGAGGCTCCACCTCTTATGGAACCGGATCAGAGACGGAAAGCCCCAGG | | |
| | AACCCTAGCAGTGCTGGAAGCTGGAACTCTGGGAGCTCTGGACCTGGAAGTACTGGAAGCT | | |
| | GGAAGCTGGAAGTACTGGAAACCAAAACCCTGGGAGCCCTAGACCTGGTAGTACCGGAACC | | |
| | TGGAATCCTGGCAGCTCTGAACGCGGAAGTGCTGGGCACTCCACCTCTGAGAGCTCTGTAT | | |
| | CTGGTAGTACTGGACAATGGCACTCTGAATCTGGAAGTTTTAGGCCAGATAGCCCAGGCTC | | |
| | TGGGAACGCGAGGCCTAACAACCCAGACTGGGGCACATTTGAAGAGGTGTCAGGAAATGTA | | |
| | AGTCCAGGGACAAGAGAGAGTACACACAGAAAACTGGTCCTTCTACAAGAGATAAGAGCTC | | |
| | GGACTGGTAAGAGAGGTCACTCTGGTACACAACACACGCGTGTCATCTCTAAACGTACTAG | | |
| | ACGTATGGCCGATGTCCAGAGTACAGAATGGAACCCAATGTCACTCCAGAAGATAGAATTT | | |
| | AGATTAATTAAGGTCCAAGCCGAATGCTAACTCATAAATGTTACCTAAAAATAGAAACTGA | | |
| | TAATCAATTACATAATAATAAAGATAAAGATAAAAAAAAGAATAAAAAAAA | | |
| | ORF Start: ATG at 55 | | ORF Stop: TAA at 1219 |
| | SEQ ID NO: 34 | 388 aa | MW at 43094.6kD |
| NOV9b, | MFSMRIVCLVLSVVGTAWTADSGEGDFLAEGGGVRGPRVVERHQSACKDSDWPFCSDEDWN | | |
| CG137873-03 | | | |
| Protein Sequence | YKCPSGCRMKGLIDEVNQDFTNRINKLKNSLFEYQKNNKDSHSLTTNIMEILRGDFSSANN | | |
| | RDNTYNRVSEDLRSRIEVLKRKVIEKVQHIQLLQKNVRAQLVDMKRLEVDIDIKIRSCRGS | | |
| | CSRALAREVDLKDYEDQQKQLEQVIAKDLLPSRGRQHLPLIKMKPVPDLVPGNFKSQLQKV | | |
| | PPEWKALTDMPQMRMELERPGGNEITRGGSTSYGTGSETESPRNPSSAGSWNSGSSGPCST | | |
| | GSWKLEVLETKTLGALDLVVPEPGILAALNAEVLGTGPLRALYLVVLDNGTLNLEVLGQIA | | |
| | QALGTRGLTTQTGAHLKRCQEM | | |
| | SEQ ID NO: 35 | 1734 bp | |
| NOV9c, | AATCCTTTCTTTCAGCTGGAGTGTCCTCAGGAGCCACCCCCACCCTTAGAAAAGATGTTTT | | |
| CG137873-02 | | | |
| DNA Sequence | CCATGAGGATCGTCTGCCTGGTCCTAAGTGTGGTGGGCACAGCATGGACTGCAGATAGTGG | | |
| | TGAAGGTGACTTTCTAGCTGAAGGAGGAGGCGTGCGTGCCCCAAGCGTTGTGGAAAGACAT | | |
| | CAATCTGCCTGCAAAGATTCAGACTGGCCCTTCTGCTCTGATGAAGACTGGAACTACAAAT | | |
| | GCCCTTCTGGCTGCAGGATGAAAGGGTTGATTGATGAAGTCAATCAAGATTTTACAAACAG | | |
| | AATAAATAACCTCAAAAATTCACTATTTGAATATCAGAAGAACAATAAGGATTCTCATTCC | | |
| | TTGACCACTAATATAATGGAAATTTTCAGACGCGATTTTTCCTCAGCCAATAACCGTGATA | | |
| | ATACCTACAACCGAGTGTCAGAGGATCTGAGAAGCAGAATTGAAGTCCTGAAGCGCAAAGT | | |
| | CATAGAAAAAGTACAGCATATCCAGCTTCTGCAAAAAAATCTTAGAGCTCAGTTGGTTGAT | | |
| | ATGAAACGACTGCAGGTGGACATTGATATTAAGATCCGATCTTGTCGAGGGTCATGCAGTA | | |
| | GGGCTTTAGCTCGTGAAGTAGATCTGAAGGACTATGAAGATCAGCAGAAGCAACTTGAACA | | |
| | GGTCATTGCCAAAGACTTACTTCCCTCTAGAGATAGGCAACACTTACCACTGATCAAAATG | | |
| | AAACCAGTTCCAGACTTGGTTCCCCGAAATTTTAACAGCCAGCTTCAGAAGGTACCCCCAG | | |
| | AGTCCAAGCCATTAACAGACATGCCCCAGATGAGAATGGAGTTAGAGAGACCTGGTGGAAA | | |
| | TGAGATTACTCGAGGAGGCTCCACTTCTTATGGAACCGCATCAGAGACCGAAAGCCCAAGG | | |
| | AACCCTAGCACTGCTGGAAGCTCGAACTCTGGCAGCTCTCCACCTGCAAGTACTCCAAGCT | | |
| | GGAACTCTGGGAGCTCTGGAACTGGAAGTACTGGAAACCAAAACCCTCGGAGCCCTAGACC | | |
| | TGGTAGTACCGGAACCTCGAATCCTGGCAGCTCTCAACGCGGAAGTGCTGGGCACTGGACC | | |
| | TCTGAGAGCTCTGTATCTGGTAGTACTGGACAATGGCACTCTGAATCTGGAAGTTTTAGGC | | |
| | CAGATAGCCCAGGCTCTGGGAACGCGAGGCCTAACAACCCAGACTGGGGCTCAGAATCTGG | | |
| | CATCTTCACAAATACAAAGCAATCCAGTTCTCATCACCCTGGGATACCTGAATTCCCTTCC | | |
| | CGTGGTAAATCTTCAAGTTACAGCAAACAATTTACTAGTAGCACGAGTTACAACAGAGGAG | | |
| | ACTCCACATTTGAAAGCAAGACCTATAAAATGGCAGATCAGGCCCGAAGTGAAGCCGATCA | | |
| | TGAAGGAACACATAGCACCAACAGAGCCCATGCTAAATCTCGCCCTGTCAGACGTATCCAC | | |
| | ACTTCTCCTTTGGGGAAGCCTTCCCTGTCCCCCTAGACTAAGTTAAATATTTCTGCACACT | | |
| | GTTCCCATGGCCCCTTGCATTTCCTTCTTAACTCTCTGTTACACGTCATTGAAACTACACT | | |
| | TTTTTGGTCTGTTTTTGTGCTAGACTGTAAGTTCCTTGGGCCCAGGGCCTTTGTCTGTCTC | | |
| | ATCTCTGTATTCCCAAATGCCTAACAGTACAGGCCCATGACTCAATAAATACATGTTAAAT | | |
| | GGATGAATGAATTCCTCTGAAACTCT | | |
| | ORF Start: ATG at 55 | | ORF Stop: TAG at 1498 |
| | SEQ ID NO: 36 | 481 aa | MW at 52648.5kD |

| | |
|---|---|
| NOV9c, | MFSMRIVCLVLSVVGTAWTADSGEGDFLAEGGGVRGPRVVERHQSACKDSDWPFCSDEDWN |
| CG137873-02 | |
| Protein Sequence | YKCPSGCRMKGLIDEVNQDFTNRINKLKNSLFEYQKNNKDSHSLTTNIMEILRGDFSSANN |
| | RDNTYNRVSEDLRSRIEVLKRKVIEKVQHIQLLQKNVRAQLVDMKRLEVDTDIKIRSCRCS |
| | CSRALAREVDLKDYEDQQKQLEQVIAKDLLPSRDRQHLPLIKMKPVPDLVPGNFKSQLQKV |
| | PPEWKALTDMPQMRMELERPGGNEITRGGSTSYGTGSETESPRNPSSAGSWNSGSSGPGST |
| | GSWNSGSSGTGSTGNQNPGSPRPGSTGTWNPGSSERGSAGHWTSESSVSGSTGQWHSESGS |
| | FRPDSPGSGNARPNNPDWGSESGIFTNTKESSSHHPGIAEFPSRGKSSSYSKQFTSSTSYN |
| | RGDSTFESKSYKMADEAGSEADHEGTHSTKRGHAKSRPVRGIHTSPLGKPSLSP |

Sequence comparison of the above protein sequences yields the following sequence relationships shown in Table 9B.

TABLE 9B
Comparison of NOV9a against NOV9b and NOV9c.

| Protein | NOV9a Residues/ | Identities/Similarities |
|---|---|---|
| Sequence | Match Residues | for the Matched Region |
| NOV9b | 1 . . . 289 | 260/289 (89%) |
| | 1 . . . 289 | 260/289 (89%) |
| NOV9c | 1 . . . 412 | 318/412 (77%) |
| | 1 . . . 386 | 319/412 (77%) |

Further analysis of the NOV9a protein yielded the following properties shown in Table 9C.

TABLE 9C
Protein Sequence Properties NOV9a

| | |
|---|---|
| PSort analysis: | 0.5087 probability located in outside; |
| | 0.1900 probability located in lysosome |
| | (lumen); 0.1000 probability located |
| | in endoplasmic reticulum (membrane); |
| | 0.1000 probability located in |
| | endoplasmic reticulum (lumen) |
| SignalP analysis: | Cleavage site between residues 20 and 21 |

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR82244 | Human fibrinogen A-alpha | 1 . . . 644 | 643/644 (99%) | 0.0 |
| | chain protein - Homo sapiens, | 1 . . . 644 | 643/644 (99%) | |
| | 644 aa. [WO9523868-A1, 08- | | | |
| | SEP-1995] | | | |
| AAR60020 | Fibronectin - Homo sapiens, | 1 . . . 644 | 641/644 (99%) | 0.0 |
| | 643 aa. [WO9416085-A, 21- | 1 . . . 643 | 641/644 (99%) | |
| | JUL-1994] | | | |
| AAY82891 | AlphaE subunit of human | 20 . . . 641 | 615/626 (98%) | 0.0 |
| | fibrinogen - Homo sapiens, | 1 . . . 626 | 616/626 (98%) | |
| | 847 aa. [WO200009562-A1, | | | |
| | 24-FEB-2000] | | | |
| AAR60019 | Tissue-binding hybrid protein - | 210 . . . 644 | 416/435 (95%) | 0.0 |
| | Homo sapiens, 1336 aa. | 910 . . . 1336 | 417/435 (95%) | |
| | [WO9416085-A, 21-JUL- | | | |
| | 1994] | | | |
| AAB54135 | Human pancreatic cancer | 1 . . . 307 | 301/307 (98%) | e−176 |
| | antigen protein sequence SEQ | 22 . . . 328 | 301/307 (98%) | |
| | ID NO: 587 - Homo sapiens, | | | |
| | 360 aa. [WO200055320-A1, | | | |
| | 21-SEP-2000] | | | |

In a BLAST search of public sequence datbases, the NOV9a protein was found to have homology to the proteins shown in the BLASTP data in Table 9E.

TABLE 9E
Public BLASTP Results for NOV9a

| | | NOV9a | | |
|---|---|---|---|---|
| Protein | | Residues/ | Identities/ | |
| Accession | | Match | Similarities for the | Expect |
| Number | Protein/Organism/Length | Residues | Matched Portion | Value |
| FGHUA | fibrinogen alpha chain | 1 . . . 644 | 644/644 (100%) | 0.0 |
| | precursor, short splice form | 1 . . . 644 | 644/644 (100%) | |
| | [validated] - human, 644 aa. | | | |
| P02671 | Fibrinogen alpha/alpha-E | 1 . . . 641 | 634/645 (98%) | 0.0 |
| | chain precursor [Contains: | 1 . . . 645 | 635/645 (98%) | |
| | Fibrinopeptide A] - Homo | | | |
| | sapiens (Human), 866 aa. | | | |
| P02672 | Fibrinogen alpha chain | 20 . . . 644 | 375/633 (59%) | 0.0 |
| | [Contains: Fibrinopeptide A] - | 4 . . . 596 | 442/633 (69%) | |
| | Bos taurus (Bovine), 596 aa | | | |
| | (fragment). | | | |
| Q99K47 | Fibrinogen A alpha | 1 . . . 634 | 371/637 (58%) | 0.0 |
| | polypeptide - Mus musculus | 1 . . . 557 | 436/637 (68%) | |
| | (Mouse), 557 aa. | | | |
| P06399 | Fibrinogen alpha/alpha-E | 1 . . . 626 | 359/629 (57%) | 0.0 |
| | chain precursor - Rattus | 1 . . . 544 | 428/629 (67%) | |
| | norvegicus (Rat), 782 aa. | | | |

PFam analysis predicts that the NOV9a protein contains the domains shown in Table 9F.

TABLE 9F
Domain Analysis of NOV9a

| | | | |
|---|---|---|---|
| Pfam | NOV9a Match Region | Identities/ | Expect Value |
| Domain | | Similarities | |
| | | for the Matched | |
| | | Region | |

### Example 10

The NOV10 clone was analyzed, and the nucleotide and encoded polypeptide sequences are shown in Table 10A.

TABLE 10A
NOV10 Sequence Analysis

| | SEQ ID NO: 37 | 730 bp | |
|---|---|---|---|
| NOV10a, | ATGCGAACACAAGTATATGAGGGGTTGTGTAAAAATTATTTTTCTCTTGCTGTACTACAA | | |
| CG137882-01 | | | |
| DNA Sequence | AGAGATACAATCAAACTGCTTTTTTTCGACATACTGGTTTTTCTTTCTCTTTTTCTTCTC | | |
| | TTTCTTCTATTTCTTGTGGATATTATGGCTAATAACACAACAAGTTTAGGGAGTCCATGG | | |
| | CCAGAAAACTTTTGGGAGGACCTTATCATGTCCTTCACTGTATCCATGGCAATCGGGCTG | | |
| | GTACTTGGAGGATTTATTTGGGCTGTGTTCATTTGTCTGTCTCGAAGAAGAAGAGCCAGT | | |
| | GCTCCCATCTCACAGTGGAGTTCAAGCACGAGATCTAGGTCTTCTTACACCCACCGCCTC | | |
| | AACAGAACTGGATTTTACCGCCACAGTGGCTGTGAACGTCGAAGCAACCTCAGCCTGGCC | | |
| | AGTCTCACCTTCCAGCGACAAGCTTCCCTGGAACAAGCAAATTCCTTTCCAAGAAAATCA | | |
| | GAGACTCAGCTGGTGACTCTCCCTTCTTCCAATATCTCTCCCACCATCAGCACTTCCCAC | | |
| | AGTCTGAGCCGTCCTGACTACTGGTCCAGTAACAGTCTTCGAGTGGGCCTTTCAACACCG | | |
| | CCCCCACCTGCCTATGAGTCCATCATCAAGGCATTCCCAGATTCCTGAGTAGGGTGGCTT | | |
| | TTGGTTTTTG | | |
| | ORF Start: ATG at 1 | | ORF Stop: TGA at 706 |
| | SEQ ID NO: 38 | 235 aa | MW at 26592.1kD |
| NOV10a, | MRTQVYEGLCKNYFSLAVLQRDRIKLLFFDILVFLSVFLLFLLFLVDIMANNTTSLGSPW | | |
| CG137882-01 | | | |
| Protein Sequence | PENFWEDLIMSFTVSMAIGLVLGGFIWAVFICLSRRRRASAPTSQWSSSRRSRSSYTHGL | | |
| | NRTGFYRHSCCERRSNLSLASLTFQRQASLEQANSFPRKSSFRASTFHPFLQCPPLPVET | | |
| | ESQLVTLPSSNISPTISTSHSLSRPDYWSSNSLRVGLSTPPPPAYESIIKAFPDS | | |
| | SEQ ID NO: 39 | 630 bp | |
| NOV10b, | ATGCGAACACAAGTATATGAGGGGTTGTGTAAAAATTATTTTTCTCTTGCTGTACTACAA | | |
| CG137882-02 | | | |
| DNA Sequence | AGAGATACAATCAAACTCCTTTTTTTCGACATACTGGTTTTTCTTTCTGTTTTTCTTCTC | | |
| | TTTCTTCTATTTCTTGTGGATATTATGGCTAATAACACAACAAGTTTAGGGAGTCCATGG | | |
| | CCAGAAAACTTTTGGGAGGACCTTATCATGTCCTTCACTGTATCCATGGCAATCGGGCTG | | |
| | GTTCTTGGAGGATTTATTTGGGCTGTGTTCATTTGTCTGTCTCGAAGAAGAAGAGCCAGT | | |
| | GCTCCCATCTCACAGTGCAGTTCAAGCAGGAGATCTAGGTCTTCTTACACCCACGGCCTC | | |
| | AACAGAACTGGATTTTACCGCCACAGTGGCTGTGAACGTCGAAGCAACCTCAGCCTGGCC | | |
| | AGTCTCACCTTCCAGCGACAAGCTTCCCTGGAACAAGCAAATTCCTTTCCAATATCTCTC | | |
| | CCACCATCAGCACTTCCCACAGTCTGAGCCGTCCTGACTACTGGTCCAGTAACAGTCTTC | | |
| | GAGTGGGCCTTTCAACACCGCCCCCACCTGCCTATGAGTCCATCATCAAGGCATTCCCAG | | |
| | ATTCCTGAGTAGGGTGGCTTTTGGTTTTTG | | |
| | ORF Start: ATG at 1 | | ORF Stop: TGA at 505 |
| | SEQ ID NO: 40 | 168 aa | MW at 19141.9kD |

| | |
|---|---|
| NOV10b, | MRTQVYEGLCKNYFSLAVLQRDRIKLLFFDILVFLSVFLLFLLFLVDIMANNTTSLGSPW |
| CG137882-02 | |
| Protein Sequence | PENFWEDLIMSFTVSMATGLVLGGFIWAVFICLSRRRRASAPISQWSSSRRSRSSYTHCL |
| | NRTGFYRHSGCERRSNLSLASLTFQRQASLEQANSFPISLPPSALPTV |

Sequence comparison of the above protein sequences yields the following sequence relationships shown in Table 10B.

TABLE 10B
Comparison of NOV10a against NOV10b.

| | NOV10a Residues/ | Identities/Similarities |
|---|---|---|
| Protein Sequence | Match Residues | for the Matched Region |
| NOV10b | 1 . . . 157 | 125/157 (79%) |
| | 1 . . . 157 | 125/157 (79%) |

Further analysis of the NOV10a protein yielded the following properties shown in Table 10C.

TABLE 10C
Protein Sequence Properties NOV10a

| | |
|---|---|
| PSort | 0.6000 probability located in nucleus; 0.6000 probability |
| analysis: | located in plasma membrane; 0.4000 probability located in |
| | Golgi body; 0.3000 probability |
| | located in endoplasmic reticulum (membrane) |
| SignalP | Cleavage site between residues 51 and 52 |
| analysis: | |

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAY59671 | Secreted protein 108-006-5- | 49 . . . 235 | 187/187 (100%) | e−107 |
| | 0-C2-FL - Homo sapiens, | 1 . . . 187 | 187/187 (100%) | |
| | 187 aa. [WO9940189-A2, | | | |
| | 12-AUG-1999] | | | |
| AAE01707 | Human gene 5 encoded | 70 . . . 235 | 166/166 (100%) | 1e−92 |
| | secreted protein HHBCS39, | 1 . . . 166 | 166/166 (100%) | |
| | SEQ ID NO: 119 - Homo | | | |
| | sapiens, 166 aa. | | | |
| | [WO200134767-A2, | | | |
| | 17-MAY-2001] | | | |
| AAE01676 | Human gene 5 encoded | 70 . . . 235 | 166/166 (100%) | 1e−92 |
| | secreted protein HHBCS39, | 1 . . . 166 | 166/166 (100%) | |
| | SEQ ID NO: 88 - Homo | | | |
| | sapiens, 166 aa. | | | |
| | [WO200134767-A2, | | | |
| | 17-MAY-2001] | | | |
| AAY65073 | Human 5′ EST related | 1 . . . 59 | 56/59 (94%) | 5e−24 |
| | polypeptide SEQ ID | 1 . . . 59 | 56/59 (94%) | |
| | NO: 1234 - Homo sapiens, 59 | | | |
| | aa. [WO9953051-A2, | | | |
| | 21-OCT-1999] | | | |
| AAG01373 | Human secreted protein, | 49 . . . 184 | 49/137 (35%) | 7e−11 |
| | SEQ ID NO: 5454 - Homo | 1 . . . 136 | 57/137 (40%) | |
| | sapiens, 136 aa. | | | |
| | [EP1033401-A2, | | | |
| | 06-SEP-2000] | | | |

In a BLAST search of public sequence datbases, the NOV10a protein was found to have homology to the proteins shown in the BLASTP data in Table 10E.

TABLE 10E
Public BLASTP Results for NOV10a

| | | NOV10a | | |
|---|---|---|---|---|
| Protein | | Residues/ | Identities/ | |
| Accession | | Match | Similarities for the | Expect |
| Number | Protein/Organism/Length | Residues | Matched Portion | Value |
| AAM88866 | MTLC - Homo sapiens | 1 . . . 235 | 235/235 (100%) | e−134 |
| | (Human), 235 aa. | 1 . . . 235 | 235/235 (100%) | |
| Q9H763 | CDNA: FLJ21269 fis, clone | 1 . . . 235 | 234/235 (99%) | e−133 |
| | COL01745 - Homo sapiens | 1 . . . 235 | 235/235 (99%) | |
| | (Human), 235 aa. | | | |
| CAD39158 | Hypothetical protein - Homo | 32 . . . 235 | 204/204 (100%) | e−115 |
| | sapiens (Human), 204 aa | 1 . . . 204 | 204/204 (100%) | |
| | (fragment). | | | |
| Q8TBE8 | Similar to RIKEN cDNA | 49 . . . 235 | 186/187 (99%) | e−105 |
| | 1110020B04 gene - Homo | 1 . . . 187 | 186/187 (99%) | |
| | sapiens (Human), 187 aa. | | | |
| Q8R411 | MT-MC1 - Mus musculus | 49 . . . 235 | 160/188 (85%) | 4e−90 |
| | (Mouse), 188 aa. | 1 . . . 188 | 173/188 (91%) | |

PFam analysis predicts that the NOV10a protein contains the domains shown in Table 10F.

TABLE 10F
Domain Analysis of NOV10a

| | | | |
|---|---|---|---|
| Pfam | NOV10a Match Region | Identities/ | Expect Value |
| Domain | | Similarities | |
| | | for the Matched | |
| | | Region | |

### Example 11

The NOV11 clone was analyzed, and the nucleotide and encoded polypeptide sequences are shown in Table 11A.

TABLE 11A
NOV11 Sequence Analysis

| | SEQ ID NO: 41 | 957 bp | |
|---|---|---|---|
| NOV11a, | CATCATGCTATGGAAAAAATGGAAGAATTTGTTTGTAAGGTATGGGAACCTCCGTGCCGA | | |
| CG137910-01 | | | |
| DNA Sequence | GTGATCCCTCATGATGTACTACCAGACTGGCTCAAGGATAATGACTTCCTCTTGCATGGA | | |
| | CACCCGCCTCCTATGCCTTCTTTCCGCGCCTGTTTTAAGAGCATTTTCAGAATACACACA | | |
| | GAAACAGGCAACATTTGGACACATCTCTTAGGTTGTGTATTCTTCCTGTCCCTGGGGATC | | |
| | TTTTATATGTTTCGCCCAAATATCTCCTTTGTGGCCCCTCTGCAAGAGAAGGTGGTCTTT | | |
| | GGATTATTTTTCTTAGGAGCCATTCTCTGCCTTTCTTTTTCATGGCTCTTCCACACAGTC | | |
| | TACTGCCACTCAGAGGGGGTCTCTCGGCTCTTCTCTAAACTGGATTACTCTGGTATTGCT | | |
| | CTTCTGATTATGGGAACTTTTGTTCCTTGGCTTTATTATTCTTTCTACTGTAATCCACAA | | |
| | CCTTGCTTCATCTACTTGATTGTCATCTGTGTGCTGGGCATTGCAGCCATTATAGTCTCC | | |
| | CAGTGGGACATGTTTCCCACCCCTCAGTATCGCGGAGTAAGAGCAGGAGTGTTTTTGGGC | | |
| | CTAGGCCTGAGTGGAATCATTCCTACCTTGCACTATGTCATCTCGGAGGGGTTCCTTAGG | | |
| | GCCCCCACCATAGGGCAGATAGGCTGGTTCATGCTGATGCCCAGCCTCTACATCACACGA | | |
| | GCTGCCCTGTATGCTCCCCGGATCCCCGAACGCTTTTTCCCTGGCAAATCTGACATCTCG | | |
| | TTTCACTCTCATCAGCTGTTTCATATCTTTGTGCTTGCTGGAGCTTTTGTTCACTTCCAT | | |
| | GGTCTCTCAAACCTCCAGGAGTTTCGTTTCATGATCGGCCCGCGCTGCAGTGAACAGGAT | | |
| | GCACTGTGATACCTACCAGTCTCCAGGGACTATGACCCTAAACCACGGCCTGCGCCA | | |
| | ORF Start: ATG at 10 | | ORF Stop: TGA at 907 |
| | SEQ ID NO: 42 | 299 aa | MW at 34157.9kD |

| | |
|---|---|
| NOV11a, | MEKMEEPVCKVWEGRWRVIPHDVLPDWLKDNDFLLHGHRPPMPSFRACFKSIFRIHTETG |
| CG137910-01 | |
| Protein Sequence | NIWTHLLGCVFFLCLCIFYMFRPNTSFVAPLQEKVVFGLFFLGAILCLSFSWLFHTVYCH |
| | SEGVSRLFSKLDYSGIALLTMCSFVPWLYYSFYCNPQPCFIYLIVICVLGIAAIIVSQWD |
| | MFATPQYRGVRAGVFLGLCLSGIIPTLHYVISEGFLRAATIGQIGWLMLMASLYITGAAL |
| | YAARIPERFFPGKCDIWFISHQLFHIFVVAGAFVHFHGVSNLQEFRFNIGGCCSEEDAL |

Further analysis of the NOV11a protein yielded the following properties shown in Table 11B.

TABLE 11B
Protein Sequence Properties NOV11a

| | |
|---|---|
| PSort analysis: | 0.6000 probability located in plasma membrane; |
| | 0.4000 probability located in |
| | Golgi body; 0.3000 probability located in endoplasmic |
| | reticulum (membrane); 0.3000 probability |
| | located in microbody (peroxisome) |
| SignalP analysis: | No Known Signal Sequence Predicted |

A search of the NOV11a protein against the Geneseq database, a proprietary database that contains sequences published in patents and patent publication, yielded several homologous proteins shown in Table 11C.

TABLE 11C
Geneseq Results for NOV11a

| | | NOV11a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| AAM79290 | Human protein SEQ ID NO | 42 . . . 299 | 256/258 (99%) | e−154 |
| | 1952 - Homo sapiens, 258 aa. | 1 . . . 258 | 257/258 (99%) | |
| | [WO200157190-A2, | | | |
| | 09-AUG-2001] | | | |
| ABB89913 | Human polypeptide SEQ ID | 1 . . . 299 | 238/299 (79%) | e−149 |
| | NO 2289 - Homo sapiens, | 77 . . . 375 | 269/299 (89%) | |
| | 375 aa. [WO200190304-A2, | | | |
| | 29-NOV-2001] | | | |
| AAB74699 | Human membrane associated | 1 . . . 299 | 238/299 (79%) | e−149 |
| | protein MEMAP-5 - Homo | 77 . . . 375 | 269/299 (89%) | |
| | sapiens, 375 aa. | | | |
| | [WO200112662-A2, | | | |
| | 22-FEB-2001] | | | |
| AAM79634 | Human protein SEQ ID NO | 1 . . . 299 | 238/299 (79%) | e−149 |
| | 3280 - Homo sapiens, 379 aa. | 81 . . . 379 | 269/299 (89%) | |
| | [WO200157190-A2, | | | |
| | 09-AUG-2001] | | | |
| AAM78650 | Human protein SEQ ID NO | 1 . . . 299 | 238/299 (79%) | e−149 |
| | 1312 - Homo sapiens, 375 aa. | 77 . . . 375 | 269/299 (89%) | |
| | [WO200157190-A2, | | | |
| | 09-AUG-2001] | | | |

In a BLAST search of public sequence datbases, the NOV11a protein was found to have homology to the proteins shown in the BLASTP data in Table 11D.

TABLE 11D
Public BLASTP Results for NOV11a

| | | NOV11a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q9H737 | CDNA: FLJ21432 fis, clone | 42 . . . 299 | 256/258 (99%) | e−153 |
| | COL04219 - Homo sapiens | 1 . . . 258 | 257/258 (99%) | |
| | (Human), 258 aa. | | | |
| Q91VH1 | Hypothetical 42.4 kDa protein - | 1 . . . 299 | 238/299 (79%) | e−149 |
| | Mus musculus (Mouse), 375 | 77 . . . 375 | 269/299 (89%) | |
| | aa. | | | |
| Q96A54 | Similar to CGI-45 protein | 1 . . . 299 | 238/299 (79%) | e−149 |
| | (Hypothetical 42.6 kDa | 77 . . . 375 | 269/299 (89%) | |
| | protein) - Homo sapiens | | | |
| | (Human), 375 aa. | | | |
| Q9Y360 | CGI-45 protein - Homo | 1 . . . 292 | 236/292 (80%) | e−147 |
| | sapiens (Human), 370 aa. | 77 . . . 368 | 264/292 (89%) | |
| Q9CZA0 | 2810031L11 Rik protein - Mus | 1 . . . 276 | 211/276 (76%) | e−126 |
| | musculus (Mouse), 352 aa. | 77 . . . 352 | 236/276 (85%) | |

PFam analysis predicts that the NOV11a protein contains the domains shown in Table 11E.

TABLE 11E
Domain Analysis of NOV11a

| | | Identities/ | |
|---|---|---|---|
| Pfam | NOV11a | Similarities | |
| Domain | Match Region | for the Matched Region | Expect Value |
| UPF0073 | 43 . . . 280 | 126/287 (44%) | 3.5e−125 |
| | | 220/287 (77%) | |

### Example 12

The NOV12 clone was analyzed, and the nucleotide and encoded polypeptide sequences are shown in Table 12A.

TABLE 12A
NOV12 Sequence Analysis

| | SEQ ID NO: 43 | 714 bp | |
|---|---|---|---|
| NOV12a, | TAACCCCAGAACATCTGGCACCTCTAACCCCAGACATCTGGCACCTCTAACCCCAGACAT | | |
| CG138013-01 | | | |
| DNA Sequence | CTGGCACCTCTAACCCCAGACATGCTCCTGCTGCTGCTGCCCCTGCTCTCGGCGAGGGAG | | |
| | AGGGCGGAAGGACAGACAAGTAAACTGCTGACGATGCAGAGTTCCGTGACGGTGCAGGAA | | |
| | GGCCTGTGTGTCCATGTGCCCTGCTCCTTCTCCTACCCCTCGCATGGCTGGATTTACCCT | | |
| | GGCCCAGTAGTTCATGGCTACTGGTTCCGGGAAGGGGCCAATACAGACCAGGATGCTCCA | | |
| | GTGGCCACAAACAACCCAGCTCGGGCAGTGTCGGAGGAGACTCCCGACCGATTCCACCTC | | |
| | CTTGGGGACCCACATACCAAGAATTGCACCCTGAGCATCAGAGATGCCAGAAGAAGTGAT | | |
| | GCGGGGAGATACTTCTTTCGTATGGAGAAAGGAAGTATAAAATGGAATTATAAACATCAC | | |
| | CGGCTCTCTGTGAATGTGACAGCCTTGACCCACAGCCCCAACATCCTCATCCCAGCCACC | | |
| | CTGGAGTCCGGCTGCCCCCAGAATCTGACCCACTCCTCAGTGGGGGAAGGAGAGCTCCAG | | |
| | TATGCATCCCTCAGCTTCCAGATGCTGAACCCTTCGCACTCACGGGCACAGCAGCCCACT | | |
| | GACACCGAGTACTCGGAGATCAAGATCCACAGATGAGAAACTGCAGAGACTCAC | | |
| | ORF Start: ATG at 82 | | ORF Stop: TGA at 694 |
| | SEQ ID NO: 44 | 204 aa | MW at 23190.9kD |

| | |
|---|---|
| NOV12a, | MLLLLLPLLWGRERAEGQTSKLLTMQSSVTVOEGLCVHVPCSPSYPSHGWIYPGPVVHGY |
| CG138013-01 | |
| Protein Sequence | WFREGANTDQDAPVATNNPARAVWEETRDRFHLLCDPHTKNCTLSIRDARRSDACRYFFR |
| | MEKGSIKWNYKHHRLSVNVTALTHRPNILIPGTLESGCPQNLTHSSVGEGELQYASLSPQ |
| | MVKPWDSRGQEATDTEYSEIKIHR |

Further analysis of the NOV12a protein yielded the following properties shown in Table 12B.

TABLE 12B
Protein Sequence Properties NOV12a

| | |
|---|---|
| PSort analysis: | 0.4170 probability located in lysosome (lumen); |
| | 0.3700 probability located in outside; 0.2303 |
| | probability located in microbody (peroxisome); 0.1000 |
| | probability located in endoplasmic reticulum |
| | (membrane) |
| SignalP analysis: | Cleavage site between residues 18 and 19 |

A search of the NOV12a protein against the Geneseq database, a proprietary database that contains sequences published in patents and patent publication, yielded several homologous proteins shown in Table 12C.

TABLE 12C
Geneseq Results for NOV12a

| | | Residues/ | Similarities for | |
|---|---|---|---|---|
| Geneseq | Protein/Organism/Length | Match | the Matched | |
| Identifier | [Patent #, Date] | Residues | Region | Expect Value |
| AAM49113 | Human dendritic cell | 1 . . . 165 | 164/165 (99%) | 5e−97 |
| | membrane protein Siglec-9 - | 1 . . . 165 | 164/165 (99%) | |
| | Homo sapiens, 463 aa. | | | |
| | [JP2001352977-A, | | | |
| | 25-DEC-2001] | | | |
| AAU87079 | Sialic acid-binding Ig-related | 1 . . . 165 | 164/165 (99%) | 5e−97 |
| | lectin, Siglec-BMS-L5a - | 1 . . . 165 | 164/165 (99%) | |
| | Homo sapiens, 463 aa. | | | |
| | [WO200208257-A2, | | | |
| | 31-JAN-2002] | | | |
| AAB29186 | OB binding protein like | 1 . . . 165 | 164/165 (99%) | 5e−97 |
| | protein #1 - Homo sapiens, | 31 . . . 195 | 164/165 (99%) | |
| | 444 aa. [WO200053747-A1, | | | |
| | 14-SEP-2000] | | | |
| AAB66137 | Protein of the invention #49 - | 1 . . . 165 | 164/165 (99%) | 5e−97 |
| | Unidentified, 463 aa. | 1 . . . 165 | 164/165 (99%) | |
| | [WO200078961-A1, | | | |
| | 28-DEC-2000] | | | |
| AAB87568 | Human PRO1302 - Homo | 1 . . . 165 | 164/165 (99%) | 5e−97 |
| | sapiens, 463 aa. | 1 . . . 165 | 164/165 (99%) | |
| | [WO200116318-A2, | | | |
| | 08-MAR-2001] | | | |

In a BLAST search of public sequence datbases, the NOV12a protein was found to have homology to the proteins shown in the BLASTP data in Table 12D.

TABLE 12D
Public BLASTP Results for NOV12a

| | | NOV12a | Identities/ | |
|---|---|---|---|---|
| Protein | | Residues/ | Similarities for | |
| Accession | | Match | the Matched | |
| Number | Protein/Organism/Length | Residues | Portion | Expect Value |
| AAF87223 | Sialic acid-binding | 1 . . . 165 | 164/165 (99%) | 1e−96 |
| | immunoglobulin-like lectin-9 - | 1 . . . 165 | 164/165 (99%) | |
| | Homo sapiens (Human), | | | |
| | 463 aa. | | | |
| Q9Y336 | OB binding protein-like | 1 . . . 165 | 164/165 (99%) | 1e−96 |
| | protein (Sialic acid-binding | 1 . . . 165 | 164/165 (99%) | |
| | lectin) - Homo sapiens | | | |
| | (Human), 463 aa. | | | |
| Q9BYI9 | FOAP-9 - Homo sapiens | 1 . . . 165 | 163/165 (98%) | 4e−96 |
| | (Human), 463 aa. | 1 . . . 165 | 164/165 (98%) | |
| Q9Y286 | QA79 membrane protein, | 1 . . . 165 | 132/169 (78%) | 3e−68 |
| | allelic variant AIRM-1B | 2 . . . 169 | 138/169 (81%) | |
| | precursor - Homo sapiens | | | |
| | (Human), 467 aa. | | | |
| Q9Y502 | QA79 membrane protein, | 1 . . . 140 | 109/144 (75%) | 6e−55 |
| | splice product AIRM-2 | 2 . . . 144 | 115/144 (79%) | |
| | precursor - Homo sapiens | | | |
| | (Human), 374 aa. | | | |

PFam analysis predicts that the NOV12a protein contains the domains shown in Table 12E.

TABLE 12E
Domain Analysis of NOV12a

| | | | |
|---|---|---|---|
| Pfam | NOV12a Match Region | Identities/ | Expect Value |
| Domain | | Similarities | |
| | | for the Matched | |
| | | Region | |

### Example 13

The NOV13 clone was analyzed, and the nucleotide and encoded polypeptide sequences are shown in Table 13A.

TABLE 13A
NOV13 Sequence Analysis

| | SEQ ID NO: 45 | 1240 bp | |
|---|---|---|---|
| NOV13a, | GGTCACTACGTGCTCTGGCCCCTCCACCTCCTGGGTCAAAGCACCCAAGCCAGGGCTGAG | | |
| CG138O74-O1 | | | |
| DNA Sequence | GAGGTTGGGCCAGAACCGGTTGGCGGGCAGGTGGGCACATTGTCAGAGATGGCTATCAGC | | |
| | CTCCTCTCCCTTCTTTCTCTGTTGCAGCTGCCTTGGGGTGCGGTGCAGGGGTCCAGAGCC | | |
| | ATTTCGGACCTGTTACTACTGGGCCTCACAGGTGGCCTGACTCTGTTACTGCTCCTGACA | | |
| | CTGCTGGCCTTTCCCGCCTACTCACCGCTGTTGGCTCGCGTGCCAGTGAGTGCCGGCTCA | | |
| | CCCCCCATCCCCCTCACCCCCCATCCCTACAAGTTCCATGTGGACCCCTATGCTGAGACT | | |
| | GGGTGGCTTTTTCACCAGAGCTGCAGCATCTCCCCCAAGCTCTGCTCCATCGCTGTCCAC | | |
| | GTTCCTCTTGGCAACTCCTGTTGTGTCCTGGGCAGCCTCCTGAGTGAAGGTGAGGAATCT | | |
| | CCCTCCCCTGAGCTCATCCACATCTACCAGAAATTTGACTTCAAGGCATTCTCCTTCCAG | | |
| | GCACCCAGCCACGTGGTGACAGCCACCTTCCCCTACACCACCATCCTGTCCATCTGGGTC | | |
| | GCTGCCTGCCATATCCATTCTGCCTTGGACACCTACATCAAGGGAACTGACATGATGAGT | | |
| | GACACGAGTTCTGGAAGCTTGGAGGTGAGCCCTGGTAGCCGGGAGACTTCATTTGCTACC | | |
| | GTGTCACCTGGGGAGAGCGGCCGCGGCTGGGAGGATGGTGACACCTGCAGTGAGTGCAGC | | |
| | TGCAGAGAGTCAGGTGCCAGCGGCTCCTCTTTTGAGGAGCTGGACCTGGAGGGTGAGGGG | | |
| | CCCTTGGACGAACCACGGCTGCACCCTGAGACTGAGCCCCTGGGGGCTACCAAGTGGCCC | | |
| | TGAGAGCCCAGTACCCTGAGAAGGGCAAGGAGTAACCCATGACCAGCCCCCTCCTGCGGG | | |
| | GCAGGGCTGCGGAACCGAGCAGACTCTCCAGCCATCTTCCTCCTTCTTCTGCCCCCGAGG | | |
| | GGTTCCCAGGGGACGTAACTCCCCCTGCTCTAGGCCTCTTGTGAAGCCTTCTCCTCACTG | | |
| | TCCTTTAGGCTCCCAGGGCCAAAGCAGCCAAAGACTGTATCCTGCACCAGCCCTGTGGGC | | |
| | CGACACTCCTGTTGTATCTCTTTTTCAGACTGTCACTGGAGCTTCCAGGACCCAGAATAA | | |
| | AGCCAATGACTTACTTGTTTCAAAAAAAAAAAAAAAAAAG | | |
| | ORF Start: ATG at 109 | | ORF Stop: TGA at 901 |
| | SEQ ID NO: 46 | 264 aa | MW at 28038.5kD |

| | |
|---|---|
| NOV13a, | MAISLLSLLSLLQLPWGAVQGSRAISDLLLLGLTGGLTLLLLLTLLAFAGYSGLLAGVAV |
| CG138074-01 | |
| Protein Sequence | SAGSPPIRLTPHPYKFHVEPYGETGWLFHQSCSISPKLCSIAVHVPLGKCCCVLGSLLSE |
| | GEESPSPELIJIYQKFDFKAFSFQAPSHVVTATFPYTTMLSIWVAACHINSALDTYIKGT |
| | DMMSDTSSGSLEVSPGSRETSFATVSPGESGRGWEDGDTCSECSCRESGASGSSFEELDL |
| | EGEGPLEEPRLDPETEPLGATKWP |

Further analysis of the NOV13a protein yielded the following properties shown in Table 13B.

TABLE 13B
Protein Sequence Properties NOV13a

| | |
|---|---|
| PSort analysis: | 0.4600 probability located in plasma membrane; |
| | 0.1197 probability located in microbody (peroxisome); |
| | 0.1000 probability located in endoplasmic reticulum |
| | (membrane); 0.1000 probability located in |
| | endoplasmic reticulum (lumen) |
| SignalP analysis: | Cleavage site between residues 50 and 51 |

A search of the NOV13a protein against the Geneseq database, a proprietary database that contains sequences published in patents and patent publication, yielded several homologous proteins shown in Table 13C.

TABLE 13C
Geneseq Results for NOV13a

| | | NOV13a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| AAE06730 | Human CASB765 protein - | 25 . . . 264 | 208/318 (65%) | e−100 |
| | Homo sapiens, 311 aa. | 1 . . . 311 | 215/318 (67%) | |
| | [WO200157077-A1, | | | |
| | 09-AUG-2001] | | | |
| AAU81960 | Human PRO536 - Homo | 25 . . . 263 | 174/302 (57%) | 8e−79 |
| | sapiens, 313 aa. | 1 . . . 301 | 187/302 (61%) | |
| | [WO200109327-A2, | | | |
| | 08-FEB-2001] | | | |
| AAB65173 | Human PRO536 (UNQ337) | 25 . . . 263 | 174/302 (57%) | 8e−79 |
| | protein sequence SEQ ID | 1 . . . 301 | 187/302 (61%) | |
| | NO: 97 - Homo sapiens, 313 | | | |
| | aa. [WO200073454-A1, | | | |
| | 07-DEC-2000] | | | |
| AAB94830 | Human protein sequence | 25 . . . 263 | 174/302 (57%) | 8e−79 |
| | SEQ ID NO: 15991 - Homo | 1 . . . 301 | 187/302 (61%) | |
| | sapiens, 313 aa. [EP1074617- | | | |
| | A2, 07-FEB-2001] | | | |
| AAU12370 | Human PRO536 polypeptide | 25 . . . 263 | 174/302 (57%) | 8e−79 |
| | sequence - Homo sapiens, | 1 . . . 301 | 187/302 (61%) | |
| | 313 aa. [WO200140466-A2, | | | |
| | 07-JUN-2001] | | | |

In a BLAST search of public sequence datbases, the NOV13a protein was found to have homology to the proteins shown in the BLASTP data in Table 13D.

TABLE 13D
Public BLASTP Results for NOV13a

| | | NOV13a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q99LS5 | Similar to putative secreted | 27 . . . 259 | 173/294 (58%) | 2e−81 |
| | protein (Unknown) (Protein | 3 . . . 296 | 188/294 (63%) | |
| | for MGC: 7091) - Mus | | | |
| | musculus (Mouse), 309 aa. | | | |
| Q9D7D9 | Adult male tongue cDNA, | 27 . . . 259 | 172/294 (58%) | 1e−80 |
| | RIKEN full-length enriched | 3 . . . 296 | 187/294 (63%) | |
| | library, clone: 2310012P03, | | | |
| | full insert sequence - Mus | | | |
| | musculus (Mouse), 309 aa. | | | |
| Q9Y619 | Putative secreted protein | 25 . . . 263 | 174/302 (57%) | 2e−78 |
| | ZSIG11 precursor - Homo | 1 . . . 301 | 187/302 (61%) | |
| | sapiens (Human), 313 aa. | | | |
| CAC25002 | Sequence 46 from Patent | 25 . . . 263 | 173/302 (57%) | 2e−76 |
| | WO0100806 precursor - | 1 . . . 300 | 186/302 (61%) | |
| | Homo sapiens (Human), 312 | | | |
| | aa. | | | |
| Q9UKD7 | Hypothetical 9.7 kDa protein - | 183 . . . 263 | 67/81 (82%) | 4e−30 |
| | Homo sapiens (Human), 93 | 1 . . . 81 | 69/81 (84%) | |
| | aa. | | | |

PFam analysis predicts that the NOV13a protein contains the domains shown in Table 13E.

TABLE 13E
Domain Analysis of NOV13a

| | | | |
|---|---|---|---|
| Pfam Domain | NOV13a Match | Identities/ | Expect Value |
| | Region | Similarities | |
| | | for the Matched | |
| | | Region | |

### Example 14

The NOV14 clone was analyzed, and the nucleotide and encoded polypeptide sequences are shown in Table 14A.

TABLE 14A
NOV14 Sequence Analysis

| | SEQ ID NO: 47 | 843 bp | |
|---|---|---|---|
| NOV14a, | GGGGTGGAGTGGGGTGTCATTTCCATCAAGTGTGCAGCATGGGTCTCTCTGTAGCACGCC | | |
| CG138573-01 | | | |
| DNA Sequence | ATGGCATGCTGGTGGCCGCTCCTGCTAGAGCTGTGGACAGTCATGCCCACCTGGGCTGGG | | |
| | GACGAGCTGCTCAACATCTGCATGAATGCCAAACACCACAAGAGAGTGCCCAGCCCAGAA | | |
| | GACAAGCTCTATGAGGAGTGCATCCCCTGGAAGGACAATGCCTGCTGCACCCTCACGACA | | |
| | AGCTGGGAAGCCCATCTGGATGTATCCCCACTCTACAACTTCAGCCTCTTTCACTGTGGA | | |
| | CTGCTGATGCCTGGCTGTCGGAAGCACTTCATCCAGGCTATCTGCTTCTATGAGTGCTCC | | |
| | CCAAACCTGGGGCCCTGGATCCAGCCAGTCGCCCCGAGTGGGCAGGGAGAGCGAGTTGTG | | |
| | AATGTGCCGCTGTGCCAGGAGGACTGTGAGGAGTGGTGGGAAGACTGTCGCATGTCTTAC | | |
| | ACATGCAAATCCAACTGGCGTGGTGGCTGGGACTGGAGTCAGGGGAAGAACCGCTGCCCC | | |
| | AAAGGGGCCCACTGCCTCCCTTTCTCCCATTACTTCCCCACCCCAGCTGACCTGTGTGAG | | |
| | AAGACTTGGAGCAATTCCTTCAAAGCCAGCCCTGAGCGACGGAACAGTGGGCGGTGTCTC | | |
| | CAGAAGTGGTTTGACCCTGCTCAGGGCAACCCCAATCTGGCCGTGGCCCGCCTCTTCGCC | | |
| | AGCTCTGCCCCATCCTGGGAACTGTCCTACACCATCATGGTCTCCTCCCTGTTCCTGCCG | | |
| | TTCCTTTCCTGAGAGCCCTTCTTCTCCCACTCACATTCCTGCATGTCCACCAACTGTGGG | | |
| | TCA | | |
| | ORF Start: ATG at 61 | | ORF Stop: TGA at 790 |
| | SEQ ID NO: 48 | 243 aa | MW at 27942.7kD |

| | |
|---|---|
| NOV14a, | MACWWPLLLELWTVMPTWAGDELLNICMNAKHHKRVPSPEDKLYEECTPWKDNACCTLTT |
| CG138573-01 | |
| Protein Sequence | SWEAHLDVSPLYNFSLFHCGLLMPGCRKHFIQAICFYECSPNLGPWIQPVAPSGQGERVV |
| | NVPLCQEDCEEWWEDCRMSYTCKSNWRGCWDWSQGKNRCPKGAQCLPFSHYFPTPADLCE |
| | KTWSNSFKASPERRNSGRCLQKWFEPAQGNPNVAVARLFASSAPSWELSYTIMVCSLFLP |
| | FLS |

Further analysis of the NOV14a protein yielded the following properties shown in Table 14B.

TABLE 14B
Protein Sequence Properties NOV14a

| | |
|---|---|
| PSort | 0.7480 probability located in microbody (peroxisome); |
| analysis: | 0.4420 probabilityl ocated in mitochondrial matrix space; |
| | 0.1282 probability located in mitochondrial inner membrane; |
| | 0.1282 probability located in mitochondrial |
| | intermembrane space |
| SignalP | Cleavage site between residues 20 and 21 |
| analysis: | |

A search of the NOV14a protein against the Geneseq database, a proprietary database that contains sequences published in patents and patent publication, yielded several homologous proteins shown in Table 14C.

TABLE 14C
Geneseq Results for NOV14a

| | | NOV14a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| AAE09454 | Human sbg72825FOLATEa | 1 . . . 243 | 243/250 (97%) | e−156 |
| | protein - Homo sapiens, 250 | 1 . . . 250 | 243/250 (97%) | |
| | aa. [WO200160850-A1, | | | |
| | 23-AUG-2001] | | | |
| AAB50286 | Human folate receptor II | 4 . . . 222 | 130/222 (58%) | 8e−82 |
| | protein SEQ ID NO: 6 - | 5 . . . 226 | 158/222 (70%) | |
| | Homo sapiens, 255 aa. | | | |
| | [WO200071754-A1, | | | |
| | 30-NOV-2000] | | | |
| ABG19167 | Novel human diagnostic | 19 . . . 222 | 120/207 (57%) | 7e−70 |
| | protein #19158 - Homo | 29 . . . 235 | 144/207 (68%) | |
| | sapiens, 248 aa. | | | |
| | [WO200175067-A2, | | | |
| | 11-OCT-2001] | | | |
| ABG04155 | Novel human diagnostic | 46 . . . 242 | 101/205 (49%) | 5e−54 |
| | protein #4146 - Homo | 1 . . . 204 | 128/205 (62%) | |
| | sapiens, 206 aa. | | | |
| | [WO200175067-A2, | | | |
| | 11-OCT-2001] | | | |
| ABG19166 | Novel human diagnostic | 19 . . . 153 | 66/151 (43%) | 9e−30 |
| | protein #19157 - Homo | 27 . . . 176 | 81/151 (52%) | |
| | sapiens, 187 aa. | | | |
| | [WO200175067-A2, | | | |
| | 11-OCT-2001] | | | |

In a BLAST search of public sequence datbases, the NOV14a protein was found to have homology to the proteins shown in the BLASTP data in Table 14D.

TABLE 14D
Public BLASTP Results for NOV14a

| Protein | | Residues/ | Similarities for | |
|---|---|---|---|---|
| Accession | | Match | the Matched | |
| Number | Protein/Organism/Length | Residues | Portion | Expect Value |
| Q9EQF4 | Folate receptor 3 (Folate | 1 . . . 241 | 166/242 (68%) | e−104 |
| | receptor 4) (Delta) - Mus | 1 . . . 242 | 191/242 (78%) | |
| | musculus (Mouse), 244 aa. | | | |
| P15328 | Folate receptor alpha | 7 . . . 242 | 140/246 (56%) | 1e−84 |
| | precursor (FR-alpha) (Folate | 10 . . . 255 | 169/246 (67%) | |
| | receptor 1) (Folate receptor, | | | |
| | adult) (Adult folate-binding | | | |
| | protein) (FBP) (Ovarian | | | |
| | tumor-associated antigen | | | |
| | MOv18) (KB cells FBP) - | | | |
| | Homo sapiens (Human), 257 | | | |
| | aa. | | | |
| Q9XSH1 | Membrane-bound folate | 7 . . . 239 | 138/240 (57%) | 4e−84 |
| | binding protein - Sus scrofa | 8 . . . 247 | 167/240 (69%) | |
| | (Pig), 249 aa. | | | |
| P41439 | Folate receptor gamma | 19 . . . 222 | 129/204 (63%) | 5e−82 |
| | precursor (FR-gamma) (Folate | 27 . . . 230 | 152/204 (74%) | |
| | receptor 3) - Homo sapiens | | | |
| | (Human), 243 aa. | | | |
| P35846 | Folate receptor alpha | 7 . . . 242 | 135/242 (55%) | 7e−82 |
| | precursor (FR-alpha) (Folate | 10 . . . 251 | 168/242 (68%) | |
| | receptor 1) (Folate-binding | | | |
| | protein 1) - Mus musculus | | | |
| | (Mouse), 255 aa. | | | |

PFam analysis predicts that the NOV14a protein contains the domains shown in Table 14E.

TABLE 14E
Domain Analysis of NOV14a

| | | Identities/ | |
|---|---|---|---|
| Pfam | | Similarities for | Expect |
| Domain | NOV14a Match Region | the Matched Region | Value |
| Folate_rec | 4 . . . 238 | 133/243 (55%) | 4e−110 |
| | | 181/243 (74%) | |

### Example 15

The NOV15 clone was analyzed, and the nucleotide and encoded polypeptide sequences are shown in Table 15A.

TABLE 15A
NOV15 Sequence Analysis

| | SEQ ID NO: 49 | 1885 bp | |
|---|---|---|---|
| NOV15a, | TCCTCAAATACAATGCTTCAAAAAACGCTGCTGATCTTGATCTCTTTTTCAGTAGTAACC | | |
| CG138606-01 | | | |
| DNA Sequence | TGGATGATTTTTATAATTTCTCAGAACTTCACAAAGCTTTGGTCTGCTCTAAACTTATCC | | |
| | ATCTCTGTCCATTACTGGAACAACTCCGCAAAGTCCTTATTCCCTAAAACATCACTGATA | | |
| | CCATTAAAGCCACTAACAGAGACTGAACTCAGAATAAAGGAAATCATAGAGAAACTAGAT | | |
| | CAGCAGATCCCACCCAGACCTTTCACCCATGTGAACACCACCACCAGTGCCACACACAGC | | |
| | ACAGCCACCATCCTCAACCCTCGAGATACATACTGCAGGGGAGACCAGCTGGACATCCTA | | |
| | CTGGAGGTGAGGGACCACTTGGGACAGAGGAAGCAATATGGTGGGGATTTCCTGAGGGCC | | |
| | AGGATGTCCTCCCCAGCACTGACGGCAGGTGCTTCAGGAAAGGTGATGGACTTCAACAAT | | |
| | GGCACCTACCTGCTCAGCTTCACTCTGTTCTGGGACGGCCAGGTCTCCCTGTCTCTGCTG | | |
| | CTCATCCACCCCAGTGAAGGGGCGTCGGCTCTCTGGAGGGCAAGGAACCAAGGCTATGAT | | |
| | AAAATTATTTTCAAAGGCAAATTTGTTAATGGCACCTCTCATGTCTTCACTGAATGTGGC | | |
| | CTGACCCTAAACTCAAATGCTGAACTCTGTGAATATCTGGATGACAGAGACCAAGAAGCC | | |
| | TTCTATTGTATGAAGCCTCAACACATGCCCTGTGAGGCTCTGACCTACATGACCACCCGG | | |
| | AATAGAGAGGTATCTTATCTTACAGACAAGGAAAACAGCCTTTTCCACAGGTCCAAAGTG | | |
| | GGAGTTGAAATGATGAAGGATCGTAAACACATTGATGTCACTAATTGTAACAAGAGAGAA | | |
| | AAAATAGAAGAGACATGCCAAGTTGCAATGAAGCCTCCTGTCCCTGGTGGTTATACTTTA | | |
| | CAAGGAAAATGGATAACAACATTTTGCAACCAGGTTCAGTTAGACACAATTAAGATAAAT | | |
| | GGCTGTTTGAAAGGCAAACTCATTTACCTCCTGGGAGACTCTACACTACGTCAGTGGATC | | |
| | TACTACTTCCCCAAAGTTGTAAAAACACTGAAGTTTTTTGATCTTCATGAAACTGGAATC | | |
| | TTTAAGAAACATTTGCTTCTGGATGCAGAAAGACACACTCAGATTCAATGGAAAAAACAT | | |
| | AGCTATCCCTTCGTCACTTTCCAGCTCTACTCTCTGATACATCATGATTATATCCCTCGG | | |
| | GAAATTGACCGGCTATCAGGTGACAAAAACACAGCCATCGTCATCACCTTTGGCCAGCAC | | |
| | TTTAGACCATTTCCCATTGACATTTTTATTCGCAGGGCCATCGGTGTTCAAAAGGCTATT | | |
| | GAAAGACTGTTCCTAAGAAGCCCAGCCACTAAAGTGATTATTAAGACAGAAAACATCAGG | | |
| | GAGATGCACATAGAGACAGAGAGGTTTCGAGACTTCCATGGTTATATTCACTATCTTATC | | |
| | ATGAAGGATATTTTCAAAGACCTCAACGTGGGCATCATTGATGCCTGGGACATGACCATT | | |
| | GCATATGGCACTGACACTATCCACCCACCTGATCATGTGATTGGAAATCAGATTAACATG | | |
| | TTCTTAAACTACATTTGCTAAGGGATAAATACTATACAAAATCACTAGGAACCAATCTCT | | |
| | GCACATAATCCCACATGTATTGTAAAGTAAGTTTTACTCATTTTAGGAACTAAGGAAAAT | | |
| | AAATTTAAAAGAATCTGTTTGGGGAGGAAGGCTATGTAAGGACAATGACAACTGATAAGG | | |
| | GATGCAAAACCAAGAGAATCATTCATGAAGAATGACTATACCATGCCTGGTTCTGATGCT | | |
| | CGTTTAAAATATTAAAAAAGTTTTT | | |
| | ORF Start: ATG at 13 | | ORF Stop: TAA at 1639 |
| | SEQ ID NO: 50 | 542 aa | MW at 62656.8kD |

| | |
|---|---|
| NOV15a, | MLQKTLLILISFSVVTWMTFIISQNFTKLWSALNLSISVHYWNNSAKSLFPKTSLIPLKP |
| CG1386O6-01 | |
| Protein Sequence | LTETELRIKEIIEKLDQQIPPRPFTHVNTTTSATHSTATILNPRDTYCRGDQLDILLEVR |
| | DHLGQRKQYGGDFLRARMSSPALTAGASGKVMDFNNGTYLVSFTLFWEGQVSLSLLLIHP |
| | SEGASALWRARNQGYDKIIFKGKFVNCTSHVFTECGLTLNSNAELCEYLDDRDQEAFYCM |
| | KPQHMPCEALTYMTTRNREVSYLTDKENSLFHRSKVGVEMMKDRKHIDVTNCNKREKIEE |
| | TCQVGMKPPVPGGYTLQGKWITTFCNQVQLDTIKINGCLKGKLIYLLGDSTLRQWIYYFP |
| | KVVKTLKFFDLHETGIFKKHLLLDAERHTQIQWKKHSYPFVTFQLYSLIDHDYIPREIDR |
| | LSGDKNTAIVITFGQHFRPFPIDIFIRRAIGVQKAIERLFLRSPATKVIIKTENIREMHI |
| | ETERFGDFHGYIHYLIMKDIFKDLNVGIIDAWDMTIAYGTDTIHPPDHVIGNQINMFLNY |
| | IC |

Further analysis of the NOV15a protein yielded the following properties shown in Table 15B.

TABLE 15B
Protein Sequence Properties NOV15a

| | |
|---|---|
| PSort | 0.6850 probability located in plasma membrane; 0.6400 |
| analysis: | probability located inendoplasmic reticulum (membrane); |
| | 0.3700 probability located in Golgi body; 0.2923 probability |
| | located in microbody (peroxisome) |
| SignalP | Cleavage site between residues 19 and 20 |
| analysis: | |

A search of the NOV15a protein against the Geneseq database, a proprietary database that contains sequences published in patents and patent publication, yielded several homologous proteins shown in Table 15C.

TABLE 15C
Geneseq Results for NOV15a

| | | Residues/ | Identities/ | |
|---|---|---|---|---|
| Geneseq | Protein/Organism/Length | Match | Similarities for the | Expect |
| Identifier | [Patent #, Date] | Residues | Matched Region | Value |
| AAU96185 | Human secreted protein, | 1 . . . 542 | 542/542 (100%) | 0.0 |
| | SEQ ID No 87 - Homo | 6 . . . 547 | 542/542 (100%) | |
| | sapiens, 547 aa. | | | |
| | [WO200224721-A1, | | | |
| | 28-MAR-2002] | | | |
| ABG27904 | Novel human diagnostic | 26 . . . 542 | 515/517 (99%) | 0.0 |
| | protein #27895 - Homo | 74 . . . 590 | 515/517 (99%) | |
| | sapiens, 590 aa. | | | |
| | [WO200175067-A2, | | | |
| | 11-OCT-2001] | | | |
| AAU83597 | Human PRO protein, Seq ID | 4 . . . 542 | 372/540 (68%) | 0.0 |
| | No 12 - Homo sapiens, 544 | 9 . . . 544 | 441/540 (80%) | |
| | aa. [WO200208288-A2, | | | |
| | 31-JAN-2002] | | | |
| AAU96219 | Human secreted protein, | 1 . . . 298 | 291/298 (97%) | e−170 |
| | SEQ ID No 121 - Homo | 6 . . . 303 | 291/298 (97%) | |
| | sapiens, 303 aa. | | | |
| | [WO200224721-A1, | | | |
| | 28-MAR-2002] | | | |
| AAB74709 | Human membrane associated | 4 . . . 273 | 220/270 (81%) | e−129 |
| | protein MEMAP-15 - Homo | 9 . . . 277 | 245/270 (90%) | |
| | sapiens, 277 aa. | | | |
| | [WO200112662-A2, | | | |
| | 22-FEB-2001] | | | |

In a BLAST search of public sequence datbases, the NOV15a protein was found to have homology to the proteins shown in the BLASTP data in Table 15D.

TABLE 15D
Public BLASTP Results for NOV15a

| | | NOV15a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q05004 | Brush border 61.9 kDa | 1 . . . 542 | 427/542 (78%) | 0.0 |
| | protein precursor - | 1 . . . 540 | 486/542 (88%) | |
| | Oryctolagus cuniculus | | | |
| | (Rabbit), 540 aa. | | | |
| AAH29049 | Hypothetical 46.9 kDa | 138 . . . 542 | 404/405 (99%) | 0.0 |
| | protein - Homo sapiens | 1 . . . 405 | 404/405 (99%) | |
| | (Human), 405 aa. | | | |
| Q9CX72 | 4432416J03Rik protein - Mus | 6 . . . 542 | 339/539 (62%) | 0.0 |
| | musculus (Mouse), 558 aa. | 24 . . . 558 | 416/539 (76%) | |
| Q96DL1 | CDNA FLJ25224 fis, clone | 2 . . . 292 | 205/291 (70%) | e−116 |
| | STM00905 - Homo sapiens | 18 . . . 308 | 239/291 (81%) | |
| | (Human), 365 aa. | | | |
| Q969Y0 | CDNA FLJ30102 fis, clone | 18 . . . 542 | 168/543 (30%) | 3e−69 |
| | BNGH41000137, weakly | 19 . . . 555 | 287/543 (51%) | |
| | similar to brush border 61.9 kDa | | | |
| | protein precursor | | | |
| | (Unknown) (Protein for | | | |
| | MGC: 15606) - Homo sapiens | | | |
| | (Human), 559 aa. | | | |

PFam analysis predicts that the NOV15a protein contains the domains shown in Table 15E.

TABLE 15E
Domain Analysis of NOV15a

| | | | |
|---|---|---|---|
| Pfam Domain | NOV15a | Identities/ | Expect Value |
| | Match Region | Similarities | |
| | | for the Matched | |
| | | Region | |

### Example 16

The NOV16 clone was analyzed, and the nucleotide and encoded polypeptide sequences are shown in Table 16A.

TABLE 16A
NOV16 Sequence Analysis

| | SEQ ID NO: 51 | 1638 bp | |
|---|---|---|---|
| NOV16a, | ACACGCGCCCAGCTCTGTAGCCTCCTCCGTCGACTCAGCCTTAGGTACCGGTCAGGCAAA | | |
| CG138751-01 | | | |
| DNA Sequence | ATGCGGTCCTCCCTGGCTCCGGGAGTCTGGTTCTTCCGGGCCTTCTCCAGGCACAGCTCG | | |
| | TTCCGAGGCCTCATCCTGCTGCTGACCTTCCTAATTTACGCCTGCTATCACATGTCCAGG | | |
| | AAGCCTATCAGTATCGTCAAGAGCCGTCTGCACCAGAACTGCTCGGAGCAGATCAAACCC | | |
| | ATCAATGATACTCACAGTCTCAATGACACCATGTGGTGCAGCTGGGCCCCATTTGACAAG | | |
| | GACAACTATAAGGAGTTACTAGGGGGCGTGGACAACGCCTTCCTCATCGCCTATGCCATC | | |
| | GGCATGTTCATCAGTGGGGTTTTTCGGGAGCGGCTTCCGCTCCGTTACTACCTCTCACCT | | |
| | GGAATGCTGCTCAGTGGCCTTTTCACCTCGCTCTTTGGCCTGGGATATTTCTGGAACATC | | |
| | CACGAGCTCTGGTACTTTGTGGTCATCCAGGTCTGTAATGGACTCGTCCAGACCACAGGC | | |
| | TGGCCCTCTGTGGTGACCTGTGTTGGCAACTGGTTCGGGAAGGGGAAGCGGGGGTTCATC | | |
| | ATGGGCATCTGGAATTCCCACACATCTGTGGGCAACATCCTGGGCTCCCTGATCGCCGGC | | |
| | ATCTGGGTGAACGCGCAGTGGCGCCTGTCGTTCATCGTCCCTGGCATCATTACTGCCGTC | | |
| | ATGGGCGTCATCACCTTCCTCTTCCTCATCGAACACCCAGAAGATGTGGACTGCGCCCCT | | |
| | CCTCAGCACCACGGTGAGCCAGCTGAGAACCAGGACAACCCTGAGGACCCTGGGAACAGT | | |
| | CCCTGCTCTATCAGGGAGAGCGGCCTTGAGACTGTGGCCAAATGCTCCAAGGGGCCATGC | | |
| | GAAGAGCCTCCTGCCATCAGCTTCTTTGGCGCGCTCCCGATCCCAGGCGTGGTCGAGTTC | | |
| | TCTCTGTGTCTGCTGTTTGCCAAGCTGGTCAGTTACACCTTCCTCTACTGGCTGCCCCTC | | |
| | TACATCGCCAATGTGGCTCACTTTAGTGCCAAGGAGGCTGGGGACCTGTCTACACTCTTC | | |
| | GATGTTGGTGGCATCATAGGCGGCATCGTGGCAGGGCTCGTCTCTGACTACACCAATGGC | | |
| | AGGGCCACCACTTGCTGTGTCATGCTCATCTTGGCTGCCCCCATGATGTTCCTGTACAAC | | |
| | TACATTGCCCAGGACGGGATTGCCAGCTCCATAGGTGAGGTCCCAGTGATGCTGATCATC | | |
| | TGTGCGGGCCTGGTCAATGGCCCATACCCGCTCATCACCACTGCTGTCTCTCCTGATCTG | | |
| | GGGACTCACAAGAGCCTGAAGGGCACAGCCAAAGCCCTGTCCACGGTCACGGCCATCATT | | |
| | GACCGCACCGGCTCCATAGGTGCGGCTCTGGGGCCTCTGCTGGCTGGGCTCATCTCCCCC | | |
| | ACGGGCTGGAACAATGTCTTCTACATGCTCATCTCTGCCGACGTCCTAGCCTGCTTGGTC | | |
| | CTTTGCCGGTTAGTATACAAAGAGATCTTGGCCTGGAAGGTGTCCCTGAGCAGAGGCAGC | | |
| | GGGTGAGTCCGGGGAGCTGAAGCTGCCCCTCTACCAACCTCATTTCTCGTGGGAATCAGC | | |
| | CCAGCGCTCAGTTTCTCC | | |
| | ORF Start: ATG at 61 | | ORF Stop: TGA at 1564 |
| | SEQ ID NO: 52 | 501 aa | MW at 54257.6kD |
| NOV16a, | MRSSLAPGVWFFRAPSRDSWFRGLILLLTFLIYACYHMSRKPISIVKSRLHQNCSEQIKP | | |
| CG138751-01 | | | |
| Protein Sequence | INDTHSLNDTMWCSWAPFDKDNYKELLGCVDNAFLIAYAIGMFISGVFGERLPLRYYLSA | | |
| | GMLLSGLFTSLFGLGYFWNIHELWYFVVIQVCNGLVQTTGWPSVVTCSGNWFGKGKRGFI | | |
| | MGIWNSHTSVGNILGSLIAGIWVNGQWGLSFIVPGIITAVMGVITFLFLIEHPEDVDCAP | | |
| | PQHHGEPAENQDNPEDPGNSPCSIRESGLETVAKCSKGPCEEPAAISFFGALRIPGVVEF | | |
| | SLCLLFAKLVSYTFLYWLPLYIANVAHFSAKEAGDLSTLFDVGGIIGGIVAGLVSDYTNG | | |
| | RATTCCVMLILAAPMMFLYNYIGQDGIASSIGEVPVMLIICGGLVNGPYALITTAVSADL | | |
| | GTHKSLKGTAKALSTVTAIIDGTGSIGAALGPLLAGLISPTGWNNVFYMLISADVLACLV | | |
| | LCRLVYKEILAWKVSLSRGSG | | |
| | SEQ ID NO: 53 | 1573 bp | |
| NOV16b, | GACTCAGCCTTAGGTACCGGTCAGGCAAAATGCGGTCCTCCCTGGCTCCGGGAGTCTGGT | | |
| CG138751-02 | | | |
| DNA Sequence | TCTTCCGGGCCTTCTCCAGGGACAGCTCGTTCCGAGGCCTCATCCTCCTGCTGACCTTCC | | |
| | TAATTTACGCCTGCTATCACATCTCCACCAAGCCTATCAGTATCGTCAAGAGCCGTCTGC | | |
| | ACCAGAACTGCTCGGAGCAGATCAAACCCATCAATGATACTCACAGTCTCAATGACACCA | | |
| | TGTGCTGCACCTGGGCCCCATTTGACAAGGACAACTATAACGAGTTACTAGCGGGCGTCG | | |
| | ACAACGCCTTCCTCATCGCCTATGCCATCGGCATGTTCATCAGTGGGGTTTTTGGGCAGC | | |
| | GGCTTCCGCTCCGTTACTACCTCTCAGCTGGAATGCTGCTCACTGGCCTTTTCACCTCGC | | |
| | TGTTTGGCCTGGGATATTTCTGGAACATCCACCAGCTCTCGTACTTTGTGGTCATCCAGG | | |
| | TCTGTAATGGACTCGTCCAGACCACAGGCTCGCCCTCTGTGGTGACCTGTGTTGCCAACT | | |
| | GGTTCGGGAAGGGGAACCCGGGGTTCATCATGGGCATCTGGAATTCCCACACATCTGTGG | | |
| | GCAACATCCTCGGCTCCCTGATCGCCGGCATCTGGGTGAACGGGCACTGGGGCCTGTCGT | | |
| | TCATCGTGCCTGCCATCATTACTGCCGTCATGGGCGTCATCACCTTCCTCTTCCTCATCG | | |
| | AACACCCAGAAGATGTGGACTGCGCCCCTCCTCAGCACCACGGTGAGCCAGCTGAGAACC | | |
| | AGGACAACCCTGAGGACCCTGGGAACAGTCCCTGCTCTATCAGGGAGAGCGGCCTTGAGA | | |
| | CTGTGGCCAAATGCTCCAAGGGGCCATGCGAAGAGCCTGCTGCCATCACCTTCTTTGGGG | | |
| | CGCTCCGGATCCCAGGCGTGGTCGAGTTCTCTCTGTGTCTGCTGTTTGCCAAGCTGGTCA | | |
| | GTTACACCTTCCTCTACTGGCTGCCCCTCTACATCGCCAATGTGGCTCACTTTACTGCCA | | |
| | AGGAGGCTGGGGACCTGTCTACACTCTTCGATGTTGGTGGCATCATAGGCGGCATCGTGG | | |
| | CAGGGCTCGTCTCTGACTACACCAATGGCAGGCCCACCACTTCCTCTGTCATGCTCATCT | | |
| | TGGCTGCCCCCATGATGTTCCTGTACAACTACATTGCCCAGGACGGGATTGCCACCTCCA | | |
| | TAGTGATCCTGATCATCTGTGGGGGCCTGGTCAATGGCCCATACGCGCTCATCACCACTG | | |
| | CTGTCTCTGCTGATCTGGGGACTCACAACAGCCTGAACGGCAACGCCAAAGCCCTGTCCA | | |
| | CGGTCACGGCCATCATTGACCGCACCGCCTCCATAGGTCCGGCTCTGGGGCCTCTGCTGG | | |
| | CTGGGCTCATCTCCCCCACGGGCTGGAACAATGTCTTCTACATCCTCATCTCTGCCGACG | | |
| | TCCTAGCCTGCTTCCTCCTTTGCCGCTTAGTATACAAAGAGATCTTCGCCTCCAACGTGT | | |
| | CCCTGAGCAGAGGCAGCGGGTGAGTCCGGGGAGCTGAAGCTGCCCCTCTACCAACCTCAT | | |
| | TTCTCGTGGGAAT | | |
| | ORF Start: ATG at 30 | | ORF Stop: TGA at 1521 |
| | SEQ ID NO: 54 | 497 aa | MW at 53902.2kD |

| | |
|---|---|
| NOV16bM | MRSSLAPCVWFFRAFSRDSWFRGLILLLTFLIYACYHMSRKPISIVKSRLHQNCSEQIKP |
| CG18751-02 | |
| Protein Sequence | INDTHSLNDTMWCSWAPFDKDNYKELLGGVDNAFLIAYAIGMFISCVFGERLPLRYYLSA |
| | GMLLSGLFTSLFGLGYFWNIHELWYFVVIQVCNGLVQTTGWPSVVTCVCTWFGKGKRGFI |
| | MGIWNSHTSVGNILGSLIAGIWVNGQWGLSFIVPGIITAVDGVITFLFLIEHPEDVDCAP |
| | PQHHGEPAENQDNPEDPGNSPCSIRESGLETVAKCSKGPCEEPAAISFFGALRIPGVVEF |
| | SLCLLFAKLVSYTFLYWLPLYIANVAHFSAKEACDLSTLFDVGGIIGGIVAGLVSDYTNG |
| | RATTCCVMLILAAPMMFLYNYIGQDGIASSIVMLIICGGLVNGPYALITTAVSADLGTHK |
| | SLKGNAKALSTVTAIIDGTGSIGAALGPLLAGLISPTGWNNVFYMLISADVLACLLLCRL |
| | VYKEILAWKVSLSRGSG |

Sequence comparison of the above protein sequences yields the following sequence relationships shown in Table 16B.

TABLE 16B
Comparison of NOV16a against NOV16b.

| | NOV16a Residues/Match | Identities/Similarities |
|---|---|---|
| Protein Sequence | Residues | for the Matched Region |
| NOV16b | 1 . . . 501 | 450/501 (89%) |
| | 1 . . . 497 | 451/501 (89%) |

Two polymorphic variants of NOV16a have been identified and are shown in Table 41D. Further analysis of the NOV16a protein yielded the following properties shown in Table 16C.

TABLE 16C
Protein Sequence Properties NOV16a

| | |
|---|---|
| PSort | 0.6318 probability located in mitochondrial inner membrane; |
| analysis: | 0.6000 probability located in plasma membrane; |
| | 0.4778 probability located in mitochondrial intermembrane |
| | space; 0.4262 probability located in mitochondrial |
| | matrix space |
| SignalP | Cleavage site between residues 37 and 38 |
| analysis: | |

A search of the NOV16a protein against the Geneseq database, a proprietary database that contains sequences published in patents and patent publication, yielded several homologous proteins shown in Table 16D.

TABLE 16D
Geneseq Results for NOV16a

| | | NOV16a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| AAM00776 | Human bone marrow protein, | 181 . . . 391 | 205/211 (97%) | e−118 |
| | SEQ ID NO: 139 - Homo | 1 . . . 211 | 206/211 (97%) | |
| | sapiens, 211 aa. | | | |
| | [WO200153453-A2, | | | |
| | 26-JUL-2001] | | | |
| AAM00889 | Human bone marrow protein, | 170 . . . 368 | 193/199 (96%) | e−113 |
| | SEQ ID NO: 365 - Homo | 3 . . . 201 | 195/199 (97%) | |
| | sapiens, 201 aa. | | | |
| | [WO200153453-A2, | | | |
| | 26-JUL-2001] | | | |
| AAG31980 | Arabidopsis thaliana protein | 24 . . . 489 | 220/470 (46%) | e−110 |
| | fragment SEQ ID NO: 38498 - | 31 . . . 462 | 296/470 (62%) | |
| | Arabidopsis thaliana, 476 | | | |
| | aa. [EP1033405-A2, | | | |
| | 06-SEP-2000] | | | |
| AAB42327 | Human ORFX ORF2091 | 295 . . . 489 | 185/195 (94%) | e−100 |
| | polypeptide sequence SEQ | 2 . . . 192 | 187/195 (95%) | |
| | ID NO: 4182 - Homo sapiens, | | | |
| | 192 aa. [WO200058473-A2, | | | |
| | 05-OCT-2000] | | | |
| ABB64855 | Drosophila melanogaster | 145 . . . 491 | 192/352 (54%) | 4e−98 |
| | polypeptide SEQ ID NO | 80 . . . 421 | 232/352 (65%) | |
| | 21357 - Drosophila | | | |
| | melanogaster, 432 aa. | | | |
| | [WO200171042-A2, | | | |
| | 27-SEP-2001] | | | |

In a BLAST search of public sequence datbases, the NOV16a protein was found to have homology to the proteins shown in the BLASTP data in Table 16E.

TABLE 16E
Public BLASTP Results for NOV16a

| | | NOV16a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q8TED4 | CDNA FLJ23627 fis, clone | 1 . . . 501 | 494/501 (98%) | 0.0 |
| | ADSU02391, highly similar | 1 . . . 497 | 495/501 (98%) | |
| | to Mus musculus cAMP | | | |
| | inducible 2 protein (Ci2) | | | |
| | mRNA - Homo sapiens | | | |
| | (Human), 501 aa. | | | |
| Q9WU81 | cAMP inducible 2 protein - | 1 . . . 501 | 435/501 (86%) | 0.0 |
| | Mus musculus (Mouse), 501 | 1 . . . 497 | 461/501 (91%) | |
| | aa. | | | |
| Q8TEM2 | FLJ00171 protein - Homo | 1 . . . 346 | 346/346 (100%) | 0.0 |
| | sapiens (Human), 396 aa | 12 . . . 357 | 346/346 (100%) | |
| | (fragment). | | | |
| Q8R070 | Similar to solute carrier | 5 . . . 489 | 308/516 (59%) | e−173 |
| | family 37 (glycerol-3- | 4 . . . 515 | 377/516 (72%) | |
| | phosphate transporter), | | | |
| | member 1 - Mus musculus | | | |
| | (Mouse), 531 aa. | | | |
| AAF46705 | CG10069-PA - Drosophila | 17 . . . 491 | 257/489 (52%) | e−136 |
| | melanogaster (Fruit fly), 516 | 30 . . . 505 | 320/489 (64%) | |
| | aa. | | | |

PFam analysis predicts that the NOV16a protein contains the domains shown in Table 16F.

TABLE 16F
Domain Analysis of NOV16a

| | | Identities/ | |
|---|---|---|---|
| | NOV16a | Similarities | Expect |
| Pfam Domain | Match Region | for the Matched Region | Value |
| sugar_tr | 9 . . . 494 | 66/553 (12%) | 0.28 |
| | | 308/553 (56%) | |

### Example 17

The NOV17 clone was analyzed, and the nucleotide and encoded polypeptide sequences are shown in Table 17A.

TABLE 17A
NOV17 Sequence Analysis

| | SEQ ID NO: 55 | 5590 bp | |
|---|---|---|---|
| NOV17a, | CTGCGGCCGGCCCCCGAGCTAGCCTGGGTTTTTTTTTTTCTCCCCTCCCTCCCCCCTTTT | | |
| CC139062-01 | | | |
| DNA Sequence | TCCATGCAGCTGATCTAAAAGGGAATAAAAGGCTGCGCATAATCATAATAATAAAAGAAG | | |
| | GGGAGCGCGAGAGAAGGAAAGAAAGCCGGGAGGTGGAAGAGGAGGGGGAGCGTCTCAAAG | | |
| | AAGCCATCAGAATAATAAAAGGAGGCCGCGCTCTTTGCCTTCTGCAACGGGCCGCTCTTG | | |
| | AAAGGGCTTTTGAAAAGTGGTGTTGTTTTCCAGTCGTGCATGCTCCAATCGGCGGAGTAT | | |
| | ATTAGAGCCGGGACGCGGCGGCCGCACGGGCAGCGGCGACGGCAGCACCGCCGCCAGCAC | | |
| | CAGCGCGAACAGCAGCGGCGGCGTCCCGAGTGCCCGCGGCCCCCCGCGCAGCGATGCGTT | | |
| | CCCCACGGACGCGCGGCCGGTCCGGGCGCCCCCTAAGCCTCCTGCTCGCCCTGCTCTGTC | | |
| | CCCTCCGAGCCAAGGTGTGTGGGGCCTCCCGTCAGTTCGACTTGGAGATCCTGTCCATGC | | |
| | AGAACGTGAACGGGCAGCTCCACAACGGGAACTGCTGCGCCGGCGCCCGCAACCCGGGAG | | |
| | ACCGCAAGTGCACCCGCGACGAGTGTGACACATACTTCAAAGTGTGCCTCAAGGAGTATC | | |
| | AGTCCCGCGTCACGGCCGGGGGGCCCTGCAGCTTCGGCTCAGGGTCCACGCCTGTCATCG | | |
| | GGGGCAACACCTTCAACCTCAAGGCCAGCCGCGGCAACGACCGCAACCGCATCGTGCTGC | | |
| | CTTTCAGTTTCGCCTGGCCGAGGTCCTATACGTTGCTTGTCGAGCCGTGGGATTCCAGTA | | |
| | ATGACACCGTTCAACCTGACAGTATTATTGAAAAGGCTTCTCACTCGGGCATGATCAACC | | |
| | CCAGCCCGCAGTGCCACACGCTGAAGCAGAACACGGGCGTTGCCCACTTTGAGTATCAGA | | |
| | TCCGCGTGACCTGTGATGACTACTACTATGGCTTTGGCTGCAATAAGTTCTGCCGCCCCA | | |
| | GAGATGACTTCTTTGGACACTATGCCTGTGACCAGAATGGCAACAAAACTTGCATGGAAG | | |
| | GCTGGATGGGCCCCGAATGTAACAGAGCTATTTGCCGACAAGGCTGCAGTCCTAAGCATG | | |
| | GGTCTTGCAAACTCCCAGGTGACTGCAGGTCCCAGTATGGCTGGCAACGCCTGTACTGTG | | |
| | ATAAGTGCATCCCACACCCGGGATCCGTCCACCGCATCTGTAATGAGCCCTGGCAGTCCC | | |
| | TCTGTGAGACCTACTGGGCCGGCCACCTCTGTGACAAAGATCTCAATTACTGTGGCACTC | | |
| | ATCAGCCGTGTCTCAACGGGGGAACTTGTAGCAACACAGGCCCTGACAAATATCAGTGTT | | |
| | CCTGCCCTGAGGGGTATTCAGGACCCAACTGTGAAATTCCTGAGCACGCCTGCCTCTCTG | | |
| | ATCCCTCTCACAACAGAGGCACCTCTAAGGAGACCTCCCTGGGCTTTGAGTGTGAGTGTT | | |
| | CCCCAGGCTGGACCGGCCCCACATGCTCTACAAACATTGATGACTGTTCTCCTAATAACT | | |
| | GTTCCCACGGGGGCACCTGCCACCACCTGGTTAACGGATTTAAGTGTGTGTGCCCCCCAC | | |
| | AGTGGACTGGGAAAACGTGCCAGTTAGATGCAAATGAATGTGAGGCCAAACCTTGTGTAA | | |
| | ACGCCAAATCCTGTAAGAATCTCATTGCCAGCTACTACTGCGACTGTCTTCCCGGCTGGA | | |
| | TGGGTCAGAATTGTGACATAAATATTAATGACTGCCTTGGCCAGTGTCAGAATGACGCCT | | |
| | CCTGTCGGGATTTGGTTAATGGTTATCGCTGTATCTGTCCACCTGGCTATGCAGGCGATC | | |
| | ACTGTGAGAGAGACATCGATGAATGTGCCAGCAACCCCTGTTTGAATGGGGGTCACTGTC | | |
| | AGAATGAAATCAACAGATTCCAGTGTCTGTGTCCCACTGGTTTCTCTGGAAACCTCTGTC | | |
| | AGCTGGACATCGATTATTGTGAGCCTAATCCCTGCCAGAACGGTGCCCAGTGCTACAACC | | |
| | GTGCCAGTGACTATTTCTGCAAGTGCCCCGAGGACTATGAGGGCAAGAACTGCTCACACC | | |
| | TGAAAGACCACTGCCGCACGACCCCCTGTGAAGTGATTGACAGCTGCACAGTGGCCATGG | | |
| | CTTCCAACGACACACCTGAAGGGGTCCGGTATATTTCCTCCAACGTCTGTGGTCCTCACG | | |
| | GGAAGTGCAAGAGTCAGTCGGGAGGCAAATTCACCTGTGACTGTAACAAAGGCTTCACGG | | |
| | GAACATACTGCCATGAAAATATTAATGACTGTGAGAGCAACCCTTGTAGAAACGGTGGCA | | |
| | CTTGCATCGATGGTGTCAACTCCTACAAGTGCATCTGTAGTGACGGCTGGGAGGGGGCCT | | |
| | ACTGTGAAACCAATATTAATGACTGCAGCCAGAACCCCTGCCACAATGGGCCCACGTGTC | | |
| | GCGACCTGGTCAATGACTTCTACTGTGACTGTAAAAATGGGTGGAAAGGAAAGACCTGCC | | |
| | ACTCACGTGACAGTCAGTGTGATGAGGCCACGTGCAACAACGGTGGCACCTGCTATGATG | | |
| | AGGGGGATGCTTTTAAGTGCATGTGTCCTGGCGGCTGGGAAGGAACAACCTGTAACATAG | | |
| | CCCGAAACAGTAGCTGCCTGCCCAACCCCTGCCATAATGGGGGCACATGTGTGGTCAACG | | |
| | GCGAGTCCTTTACGTGCGTCTGCAAGGAAGGCTGGGAGGGGCCCATCTGTGCTCAGAATA | | |
| | CCAATGACTGCAGCCCTCATCCCTGTTACAACAGCGGCACCTGTGTGGATGGAGACAACT | | |
| | GGTACCGGTGCGAATGTGCCCCGGGTTTTGCTGGGCCCGACTGCAGAATAAACATCAATG | | |
| | AATGCCAGTCTTCACCTTGTGCCTTTGGAGCGACCTGTGTGGATGAGATCAATGGCTACC | | |
| | GGTCTGTCTGCCCTCCAGGGCACAGTGGTGCCAAGTGCCAGGAAGTTTCAGCCAGACCTT | | |
| | GCATCCACCATGGGAGTGTGATACCAGATGGGGCCAAATGGGATGATGACTGTAATACCT | | |
| | GCCAGTGCCTGAATGGACGGATCGCCTGCTCAAAGCTCTGGTGTGGCCCTCGACCTTGCC | | |
| | TGCTCCACAAAGGCCACAGCGAGTGCCCCAGCGGGCAGAGCTGCATCCCCATCCTGGACG | | |
| | ACCAGTGCTTCGTCCACCCCTGCACTGGTGTGGGCGAGTGTCGGTCTTCCAGTCTCCAGC | | |
| | CGGTGAAGACAAAGTGCACCTCTGACTCCTATTACCAGGATAACTGTGCGAACATCACAT | | |
| | TTACCTTTAACAAGGAGATGATGTCACCAGGTCTTACTACGGAGCACATTTGCAGTGAAT | | |
| | TGAGGAATTTGAATATTTTGAAGAATGTTTCCGCTGAATATTCAATCTACATCGCTTGCG | | |
| | AGCCTTCCCCTTCAGCGAACAATGAAATACATGTGGCCATTTCTGCTGAAGATATACGGG | | |
| | ATGATGGGAACCCGATCAAGGAAATCACTGACAAAATAATCGATCTTGTTAGTAAACGTG | | |
| | ATGCAAACAGCTCGCTGATTGCTGCCCTTGCAGAAGTAAGAGTTCAGAGGCGGCCTCTGA | | |
| | AGAACAGAACAGATTTCCTTGTTCCCTTGCTGAGCTCTGTCTTAACTGTGGCTTGGATCT | | |
| | GTTGCTTGGTGACGGCCTTCTACTGGTGCCTGCGGAAGCGGCGGAAGCCGGGCAGCCACA | | |
| | CACACTCAGCCTCTGAGGACAACACCACCAACAACGTGCGGGAGCAGCTGAACCAGATCA | | |
| | AAAACCCCATTGAGAAACATGGGGCCAACACGGTCCCCATCAAGGATTACGAGAACAAGA | | |
| | ACTCCAAAATGTCTAAAATAAGGACACACAATTCTGAAGTAGAAGAGGACGACATGGACA | | |
| | AACACCAGCAGAAAGCCCGGTTTGCCAAGCAGCCGGCGTATACGCTGGTAGACAGAGAAG | | |
| | AGAAGCCCCCCAACGGCACGCCGACAAAACACCCAAACTGGACAAACAAACAGGACAACA | | |
| | GAGACTTGGAAAGTGCCCAGAGCTTAAACCGAATGGACTACATCGTATACCAGACCCCGG | | |
| | GCACTGCCGCCGCTAGGTAGAGTCTGAGGGCTTGTAGTTCTTTAAACTGTCGTGTCATAC | | |
| | TCGAGTCTGAGGCCGTTGCTGACTTAGAATCCCTGTGTTAATTTAAGTTTTGACAAGCTG | | |
| | GCTTACACTGGCAATGGTAGTTTCTGTGGTTGGCTGGGAAATCGAGTGCCGCATCTCACA | | |
| | GCTATGCAAAAAGCTAGTCAACACTACCCTGCTTGTGTGTCCCCTTGCAGCCGACACGCT | | |
| | CTCGGATCACGCTCCCAGGAGCCTGCCCACCCCCCTGGTCTTTGAGCTCCCACTTCTGCC | | |
| | AGATGTCCTAATGGTGATGCAGTCTTAGATCATAGTTTTATTTATATTTATTGACTCTTG | | |
| | AGTTGTTTTTGTATATTGGTTTTATGATGACGTACAAGTAGTTCTGTATTTGAAAGTGCC | | |
| | TTTGCAGCTCAGAACCACAGCAACGATCACAAATGACTTTATTATTTATTTTTTTAATTG | | |
| | TATTTTTGTTGTTGGGGGAGGGGAGACTTTGATGTCAGCAGTTGCTGGTAAAATGAAGAA | | |
| | TTTAAAGAAAAAAATGTCAAAAGTAGAACTTTGTATAGTTATGTAAATAATTCTTTTTTA | | |
| | TTAATCACTGTGTATATTTGATTTATTAACTTAATAATCAAGAGCCTTAAAACATCATTC | | |
| | CTTTTTATTTATATGTATGTGTTTAGAATTGAAGGTTTTTGATAGCATTGTAAGCGTATG | | |
| | GCTTTATTTTTTTGAACTCTTCTCATTACTTGTTGCCTATAAGCCAAAATTAAGGTGTTT | | |
| | GAAAATAGTTTATTTTAAAACAATAGGATGGGCTTCTGTGCCCAGAATACTGATGGAATT | | |
| | TTTTTTGTACGACGTCAGATGTTTAAAACACCTTCTATAGCATCACTTAAAACACGTTTT | | |
| | AAGGACTGACTGAGGCAGTTTGAGGATTAGTTTAGAACACGTTTTTTTGTTTGTTTGTTT | | |
| | TTTGTTTTTCTGCTTTAGACTTGAAAAGAGACAGGCAGGTGATCTGCTGCAGAGCAGTAA | | |
| | GGGAACAAGTTGAGCTATGACTTAACATAGCCAAAATGTGAGTGGTTGAATATGATTAAA | | |
| | AATATCAAATTAATTGTGTGAACTTGGAAGCACACCAATCTGACTTTGTAAATTCTGATT | | |
| | TCTTTTCACCATTCGTACATAATACTGAACCACTTGTAGATTTGATTTTTTTTTTAATCT | | |
| | ACTGCATTTAGGGAGTATTCTAATAAGCTAGTTGAATACTTGAACCATAAAATGTCCAGT | | |
| | AAGATCACTGTTTAGATTTGCCATAGAGTACACTGCCTGCCTTAAGTGAGGAAATCAAAG | | |
| | TGCTATTACGAAGTTCAAGATCAAAAAGGCTTATAAAACAGAGT1ATCTTGTTGGTTCAC | | |
| | CATTGAGACCGTGAAGATACTTTGTATTGTCCTATTAGTGTTATATGAACATACAAATGC | | |
| | ATCTTTGATGTGTTGTTCTTGGCAATAAATTTTGAAAAGTAATATTATTAAATTTTTTTT | | |
| | GTATGAAAAC | | |
| | ORF Start: ATG at 414 | | ORF Stop: TAG at 4068 |
| | SEQ ID NO: 56 | 1218 aa | MW at 133797.1kD |
| NOV17a, | MRSPRTRGRSGRPLSLLLALLCALRAKVCGASGQFELEILSMQNVNGELQNGNCCGGARN | | |
| CG139062-01 | | | |
| Protein Sequence | PGDRKCTRDECDTYFKVCLKEYQSRVTAGGPCSFGSGSTPVIGGNTFNLKASRGNDRNRI | | |
| | VLPFSFAWPRSYTLLVEAWDSSNDTVQPDSIIEKASHSGMINPSRQWQTLKQNTGVAHPE | | |
| | YQIRVTCDDYYYGFGCNKFCRPRDDFFGHYACDQNGNKTCMEGWMGPECNRAICRQGCSP | | |
| | KHGSCKLPGDCRCQYGWQGLYCDKCIPHPGCVHGICNEPWQCLCETNWGGQLCDKDLNYC | | |
| | GTHQPCLNGGTCSNTGPDKYQCSCPEGYSGPNCEIAEHACLSDPCHNRGSCKETSLCFEC | | |
| | ECSPGWTGPTCSTNIDDCSPNNCSHGGTCQDLVNGFKCVCPPQWTGKTCQLDANECEAKP | | |
| | CVNAKSCKNLIASYYCDCLPGWMGQNCDININDCLGQCQNDASCRDLVNGYRCICPPGYA | | |
| | GDHCERDIDECASNPCLNGGHCQNEINRFQCLCPTGFSGNLCQLDIDYCEPNPCQNGAQC | | |
| | YNRASDYFCKCPEDYEGKNCSHLKDHCRTTPCEVIDSCTVAMASNDTPEGVRYISSNVCG | | |
| | PHGKCKSQSGGKFTCDCNKGFTGTYCHENINDCESNPCRNGGTCIDGVNSYKCICSDGWE | | |
| | GAYCETNINDCSQNPCHNGGTCRDLVNDFYCDCKNCWKGKTCHSRDSQCDEATCNNGGTC | | |
| | YDEGDAFKCMCPGGWEGTTCNIARNSSCLPNPCHNGGTCVVNGESFTCVCKEGWEGPICA | | |
| | QNTNDCSPHPCYNSGTCVDGDNWYRCECAPCFAGPDCRININECQSSPCAFGATCVDEIN | | |
| | GYRCVCPPGHSGAKCQEVSGRPCITMGSVIPDGAKWDDDCNTCQCLNGRIACSKVWCGPR | | |
| | PCLLHKGHSECPSGQSCIPILDDQCFVHPCTGVGECRSSSLQPVKTKCTSDSYYQDNCAN | | |
| | ITFTFNKEMMSPGLTTEHICSELRNLNILKNVSAEYSIYIACEPSPSANNEIHVAISAED | | |
| | IRDDGNPIKEITDKIIDLVSKRDGNSSLIAAVAEVRVQRRPLKNRTDFLVPLLSSVLTVA | | |
| | WICCLVTAFYWCLRKRRKPGSHTHSASEDNTTNNVREQLNQIKNPIEKHGANTVPIKDYE | | |
| | NKNSKMSKIRTHNSEVEEDDMDKHQQKARFAKQPAYTLVDREEKPPNGTPTKHPNWTNKQ | | |
| | DNRDLESAQSLNRMEYIV | | |
| | SEQ ID NO: 57 | 4333 bp | |
| NOV17b | CTGCCCCCGGCCCCCGAGCTAGGCTGCGTTTTTTTTTTTCTCCCCTCCCTCCCCCCTTTT | | |
| CG139062-02 | | | |
| DNA Sequence | TCCATGCAGCTGATCTAAAAGGGAATAAAAGGCTGCGCATAATCATAATAATAAAAGAAG | | |
| | GGGAGCGCGAGAGAAGGAAAGAAAGCCGGGAGGTGGAAGAGGAGGGGGAGCGTCTCAAAG | | |
| | AAGCGATCAGAATAATAAAAGGAGGCCGGGCTCTTTGCCTTCTGGAACGGGCCGCTCTTG | | |
| | AAAGGGCTTTTGAAAAGTGGTGTTGTTTTCCAGTCGTGCATGCTCCAATCGGCGGAGTAT | | |
| | ATTAGAGCCGGGACGCCGCGGCCGCAGGCGCAGCCGCGACGGCACCACCGGCGGCACCAC | | |
| | CACCGCGAACAGCAGCGGCGGCGTCCCGAGTGCCCGCGGCGCGCGGCGCAGCGATGCGTT | | |
| | CCCCACGGACGCGCGGCCGGTCCGGGCGCCCCCTAAGCCTCCTGCTCGCCCTGCTCTGTG | | |
| | CCCTGCGAGCCAAGGTGTGTGCGGCCTCCGGTCAGTTCCAGTTGGACATCCTGTCCATGC | | |
| | AGAACGTGAACGGGGAGCTGCAGAACGGGAACTGCTGCGGCGGCGCCCGGAACCCGGGAG | | |
| | ACCGCAAGTGCACCCGCGACGAGTGTGACACATACTTCAAAGTGTGCCTCAAGGAGTATC | | |
| | AGTCCCGCCTCACGGCCCCGGGGCCCTGCACCTTCGGCTCAGGGTCCACGCCTGTCATCG | | |
| | GGGGCAACACCTTCAACCTCAAGGCCAGCCGCGGCAACGACCGCAACCGCATCGTGCTGC | | |
| | CTTTCAGTTTCGCCTGGCCGAGGTCCTATACGTTGCTTGTGGAGCCGTGGGATTCCAGTA | | |
| | ATGACACCGTTCAACCTGACAGTATTATTGAAAAGGCTTCTCACTCGGGCATGATCAACC | | |
| | CCAGCCGGCAGTGCCAGACCCTGAAGCAGAACACGGGCGTTGCCCACTTTGAGTATCACA | | |
| | TCCGCGTGACCTGTGATGACTACTACTATGGCTTTGGCTGCAATAAGTTCTGCCGCCCCA | | |
| | GAGATGACTTCTTTGGACACTATGCCTGTGACCAGAATGGCAACAAAACTTGCATGGAAG | | |
| | GCTGGATGGGCCCCGAATGTAACAGAGCTATTTGCCGACAAGGCTGCAGTCCTAAGCATG | | |
| | GGTCTTGCAAACTCCCAGGTGACTGCAGGTGCCAGTATGGCTGGCAAGGCCTGTACTGTG | | |
| | ATAAGTGCATCCCACACCCGGGATGCGTCCACGGCATCTGTAATGAGCCCTGGCAGTGCC | | |
| | TCTGTGAGACCAACTGGGGCGGCCAGCTCTGTGACAAAGATCTCAATTACTGTGGGACTC | | |
| | ATCAGCCGTGTCTCAACGGGGGAACTTGTAGCAACACAGGCCCTGACAAATATCAGTGTT | | |
| | CCTGCCCTGAGGGGTATTCAGGACCCAACTGTGAAATTGCTGAGCACGCCTGCCTCTCTG | | |
| | ATCCCTGTCACAACAGAGGCACCTCTAACGAGACCTCCCTGGGCTTTGAGTGTGAGTGTT | | |
| | CCCCAGGCTGGACCGGCCCCACATGCTCTACAAACATTGATGACTGTTCTCCTAATAACT | | |
| | GTTCCCACGGGGGCACCTGCCAGGACCTGGTTAACGGATTTAAGTGTGTGTGCCCCCCAC | | |
| | AGTGGACTGGGAAAACGTGCCAGTTAGATGCAAATGAATGTGAGGCCAAACCTTGTGTAA | | |
| | ACGCCAAATCCTGTAAGAATCTCATTGCCAGCTACTACTGCGACTGTCTTCCCGGCTGGA | | |
| | TGGGTCAGAATTGTGACATAAATATTAATGACTGCCTTGGCCAGTGTCAGAATGACGCCT | | |
| | CCTGTCGGGATTTGGTTAATGCTTATCGCTGTATCTGTCCACCTGGCTATGCAGGCGATC | | |
| | ACTGTGAGAGAGACATCGATGAATGTGCCAGCAACCCCTGTTTGAATGGGGGTCACTGTC | | |
| | AGAATGAAATCAACAGATTCCAGTGTCTGTGTCCCACTGGTTTCTCTGGAAACCTCTGTC | | |
| | AGCTGGACATCGATTATTGTGAGCCTAATCCCTGCCAGAACGGTGCCCAGTGCTACAACC | | |
| | GTGCCAGTGACTATTTCTGCAAGTGCCCCGAGGACTATGAGGGCAAGAACTGCTCACACC | | |
| | TGAAAGACCACTGCCGCACGACCCCCTGTGAAGTGATTGACAGCTGCACAGTGGCCATGG | | |
| | CTTCCAACGACACACCTGAAGGGGTGCGGTATATTTCCTCCAACGTCTGTGGTCCTCACG | | |
| | GGAAGTGCAAGAGTCAGTCGGGAGGCAAATTCACCTGTGACTGTAACAAAGGCTTCACGG | | |
| | GAACATACTGCCATGAAAATATTAATGACTGTGAGAGCAACCCTTGTAGAAACGGTGGCA | | |
| | CTTGCATCGATGGTGTCAACTCCTACAAGTGCATCTGTAGTGACGGCTGGGAGGGCGCCT | | |
| | ACTCACGTGACAGTCAGTGTGATGAGGCCAACACGGTCCCCATCAAGGATTACGAGAACA | | |
| | GCGACCTGGTCAATGACTTCTACTGTGGCTGTAAAAATGGGTGGAAAGGAAAGACCTGCC | | |
| | ACTCACGTGACAGTCAGTGTGATGAGGCCAACACGGTCCCCATCAAGGATTACGAGAACA | | |
| | AGAACTCCAAAATGTCTAAAATAAGGACACACAATTCTGAAGTAGAAGAGGACGACATGG | | |
| | ACAAACACCAGCAGAAAGCCCGGTTTGCCAAGCAGCCGGCGTACACGCTGGTAGACAGAG | | |
| | AAGAGAAGCCCCCCAACGGCACGCCGACAAAACACCCAAACTGGACAAACAAACAGGACA | | |
| | ACAGAGACTTGGAAAGTCCCCAGAGCTTAAACCGAATGGAGTACATCGTATAGCACACCG | | |
| | CGGGCACTGCCGCCGCTAGGTAGAGTCTGAGGGCTTGTAGTTCTTTAAACTGTCGTGTCA | | |
| | TACTCGAGTCTGAGGCCGTTGCTGACTTAGAATCCCTGTGTTAATTTAAGTTTTGACAAG | | |
| | CTGGCTTACACTGGCAATGGTAGTTTCTGTGGTTGGCTGGGAAATCGAGTGCCGCATCTC | | |
| | ACAGCTATGCAAAAAGCTAGTCAACAGTACCCTGGTTGTGTGTCCCCTTGCAGCCGACAC | | |
| | GGTCTCGGATCAGGCTCCCAGGAGCCTGCCCAGCCCCCTGGTCTTTGAGCTCCCACTTCT | | |
| | GCCAGATGTCCTAATGGTGATGCAGTCTTAGATCATAGTTTTATTTATATTTATTGACTC | | |
| | TTGAGTTGTTTTTGTATATTGGTTTTATGATGACGTACAAGTAGTTCTGTATTTGAAAGT | | |
| | GCCTTTGCAGCTCAGAACCACAGCAACGATCACAAATGACTTTATTATTTATTTTTTTAA | | |
| | TTGTATTTTTGTTGTTGGGGGAGGGGAGACTTTGATGTCAGCAGTTGCTGGTAAAATGAA | | |
| | GAATTTAAAGAAAAAAATGTCAAAAGTAGAACTTTGTATAGTTATGTAAATAATTCTTTT | | |
| | TTATTAATCACTGTGTATATTTGATTTATTAACTTAATAATCAAGAGCCTTAAAACATCA | | |
| | TTCCTTTTTATTTATATGTATGTGTTTAGAATTGAAGGTTTTTGATAGCATTGTAAGCGT | | |
| | ATGGCTTTATTTTTTTGAACTCTTCTCATTACTTGTTGCCTATAAGCCAAAATTAAGGTG | | |
| | TTTGAAAATAGTTTATTTTAAAACAATAGGATGGGCTTCTGTGCCCAGAATACTGATGGA | | |
| | ATTTTTTTTGTACGACGTCAGATGTTTAAAACACCTTCTATAGCATCACTTAAAACACGT | | |
| | TTTAAGGACTGACTGAGGCAGTTTGAGGATTAGTTTAGAACAGGTTTTTTTGTTTGTTTG | | |
| | TTTTTTGTTTTTCTGCTTTAGACTTGAAAAGAGACAGGCACGTGATCTCCTCCAGACCAG | | |
| | TAAGGGAACAAGTTGAGCTATGACTTAACATAGCCAAAATGTGAGTGGTTGAATATGATT | | |
| | AAAAATATCAAATTAATTGTGTGAACTTGGAAGCACACCAATCTGACTTTGTAAATTCTG | | |
| | ATTTCTTTTCACCATTCGTACATAATACTGAACCACTTGTAGATTTGATTTTTTTTTTAA | | |
| | TCTACTGCATTTAGGGAGTATTCTAATAAGCTAGTTGAATACTTGAACCATAAAATGTCC | | |
| | AGTAAGATCACTGTTTAGATTTGCCATAGAGTACACTGCCTGCCTTAAGTGAGGAAATCA | | |
| | AAGTGCTATTACGAAGTTCAAGATCAAAAAGGCTTATAAAACAGAGTAATCTTGTTGGTT | | |
| | CACCATTGAGACCGTGAAGATACTTTGTATTGTCCTATTAGTGTTATATGAACATACAAA | | |
| | TGCATCTTTGATGTGTTGTTCTTGGCAATAAATTTTGAAAAGTAATATTTATTAAATTTT | | |
| | TTTGTATGAAAAC | | |
| | ORF Start: ATG at 414 | | ORF Stop: TAG at 2811 |
| | SEQ ID NO: 58 | 799 aa | MW at 88212.4kD |

| | |
|---|---|
| NOV17b, | MRSPRTRGRSGRPLSLLLALLCALRAKVCGASGQFELEILSMQNVNGELQNGNCCGGARN |
| CG139062-02 | |
| Protein Sequence | PGDRKCTRDECDTYFKVCLKEYQSRVTAGGPCSPGSGSTPVIGGNTFNLKASRGNDRNRI |
| | VLPFSFAWPRSYTLLVEAWDSSNDTVQPDSIIEKASHSGMINPSRQWQTLKQNTGVAHFE |
| | YQIRVTCDDYYYGPGCNKFCRPRDDFFGHYACDQNGNKTCMEGWMGPECNRAICRQGCSP |
| | KHGSCKLPGDCRCQYGWQGLYCDKCIPHPGCVHGICNEPWQCLCETNWGGQLCDKDLNYC |
| | GTHQPCLNGGTCSNTGPDKYQCSCPEGYSGPNCEIAEHACLSDPCHNRGSCKETSLGFEC |
| | ECSPGWTGPTCSTNIDDCSPNNCSHGGTCQDLVNGFKCVCPPQWTGKTCQLDANECEAKP |
| | CVNAKSCKNLIASYYCDCLPCWMGQNCDININDCLGQCQNDASCRDLVNGYRCICPPGYA |
| | GDHCERDIDECASNPCLNGGHCQNEINRFQCLCPTGFSGNLCQLDIDYCEPNPCQNGAQC |
| | YNIASDYFCKCPEDYEGKNCSHLKDHCRTTPCEVIDSCTVAMASNDTPECVRYISSNVCG |
| | PHGKCKSQSGGKFTCDCNKGFTCTYCHENINDCESNPCRNGGTCIDGVNSYKCICSDGWE |
| | GAYCETNINDCSQNPCHNGGTCRDLVNDFYCGCKNGWKGKTCHSRDSQCDEANTVPIKDY |
| | ENKNSKMSKTRTHNSEVEEDDMDKHQQKARFAKQPAYTLVDREEKPPNGTPTKHPNWTNK |
| | QDNRDLESAQSLNRMEYIV |

Sequence comparison of the above protein sequences yields the following sequence relationships shown in Table 17B.

TABLE 17B
Comparison of NOV17a against NOV17b.

| | NOV17a Residues/ | Identities/Similarities |
|---|---|---|
| Protein Sequence | Match Residues | for the Matched Region |
| NOV17b | 27 . . . 712 | 685/686 (99%) |
| | 27 . . . 712 | 685/686 (99%) |

Five polymorphic variants of NOV17b have been identified and are shown in Table 41E.

Further analysis of the NOV17a protein yielded the following properties shown in Table 17C.

TABLE 17C
Protein Sequence Properties NOV17a

| | |
|---|---|
| PSort | 0.4600 probability located in plasma membrane; |
| analysis: | 0.1000 probability located in endoplasmic reticulum |
| | (membrane); 0.1000 probability located in |
| | endoplasmic reticulum (lumen); 0.1000 probability located |
| | in outside |
| SignalP | Cleavage site between residues 34 and 35 |
| analysis: | |

A search of the NOV17a protein against the Geneseq database, a proprietary database that contains sequences published in patents and patent publication, yielded several homologous proteins shown in Table 17D.

TABLE 17D
Geneseq Results for NOV17a

| | | NOV17a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABB07822 | Human notch agonist ligand - | 1 . . . 1218 | 1218/1218 (100%) | 0.0 |
| | Homo sapiens, 1218 aa. | 1 . . . 1218 | 1218/1218 (100%) | |
| | [WO200218544-A2, | | | |
| | 07-MAR-2002] | | | |
| AAW87894 | Human JAGGED1 protein - | 1 . . . 1218 | 1218/1218 (100%) | 0.0 |
| | Homo sapiens, 1218 aa. | 1 . . . 1218 | 1218/1218 (100%) | |
| | [WO9858958-A2, | | | |
| | 30-DEC-1998] | | | |
| AAW44301 | Human serrate 1 - Homo | 1 . . . 1218 | 1218/1218 (100%) | 0.0 |
| | sapiens, 1218 aa. | 1 . . . 1218 | 1218/1218 (100%) | |
| | [WO9802458-A1, | | | |
| | 22-JAN-1998] | | | |
| AAU84344 | Protein JAG1 differentially | 1 . . . 1218 | 1217/1218 (99%) | 0.0 |
| | expressed in breast cancer | 1 . . . 1218 | 1217/1218 (99%) | |
| | tissue - Homo sapiens, 1218 | | | |
| | aa. [WO200210436-A2, | | | |
| | 07-FEB-2002] | | | |
| AAY59597 | Human Serrate protein | 1 . . . 1218 | 1215/1218 (99%) | 0.0 |
| | sequence - Homo sapiens, | 1 . . . 1218 | 1216/1218 (99%) | |
| | 1218 aa. [US6004924-A, | | | |
| | 21-DEC-1999] | | | |

In a BLAST search of public sequence datbases, the NOV17a protein was found to have homology to the proteins shown in the BLASTP data in Table 17E.

TABLE 17E
Public BLASTP Results for NOV17a

| | | NOV17a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P78504 | Jagged 1 precursor (Jagged 1) | 1 . . . 1218 | 1218/1218 (100%) | 0.0 |
| | (hJ1) - Homo sapiens | 1 . . . 1218 | 1218/1218 (100%) | |
| | (Human), 1218 aa. | | | |
| Q9QXX0 | Jagged 1 precursor (Jagged 1) - | 1 . . . 1218 | 1176/1218 (96%) | 0.0 |
| | Mus musculus (Mouse), | 1 . . . 1218 | 1194/1218 (97%) | |
| | 1218 aa. | | | |
| Q63722 | Jagged 1 precursor (Jagged 1) - | 1 . . . 1218 | 1175/1219 (96%) | 0.0 |
| | Rattus norvegicus (Rat), | 1 . . . 1219 | 1191/1219 (97%) | |
| | 1219 aa. | | | |
| A56136 | jagged protein precursor - | 1 . . . 1218 | 1168/1223 (95%) | 0.0 |
| | rat, 1220 aa. | 1 . . . 1220 | 1184/1223 (96%) | |
| Q90819 | C-Serate-1 protein - Gallus | 27 . . . 1218 | 1047/1193 (87%) | 0.0 |
| | gallus (Chicken), 1193 aa | 1 . . . 1193 | 1111/1193 (92%) | |
| | (fragment). | | | |

PFam analysis predicts that the NOV17a protein contains the domains shown in Table 17F.

TABLE 17F
Domain Analysis of NOV17a

| | | Identities/ | |
|---|---|---|---|
| | | Similarities | |
| | NOV17a | for the Matched | |
| Pfam Domain | Match Region | Region | Expect Value |
| DSL | 167 . . . 229 | 42/67 (63%) | 3.9e−40 |
| | | 63/67 (94%) | |
| EGF | 300 . . . 333 | 18/47 (38%) | 1e−06 |
| | | 28/47 (60%) | |
| EGF | 340 . . . 371 | 16/47 (34%) | 3.3e−08 |
| | | 26/47 (55%) | |
| EGF | 378 . . . 409 | 18/47 (38%) | 2.9e−09 |
| | | 30/47 (64%) | |
| EGF | 416 . . . 447 | 13/47 (28%) | 0.003 |
| | | 19/47 (40%) | |
| EGF | 454 . . . 484 | 14/47 (30%) | 4.6e−07 |
| | | 26/47 (55%) | |
| EGF | 491 . . . 522 | 16/47 (34%) | 1.7e−07 |
| | | 24/47 (51%) | |
| EGF | 529 . . . 560 | 17/47 (36%) | 2.5e−08 |
| | | 26/47 (55%) | |
| EGF | 595 . . . 626 | 13/47 (28%) | 0.19 |
| | | 24/47 (51%) | |
| EGF | 633 . . . 664 | 15/47 (32%) | 1.3e−08 |
| | | 25/47 (53%) | |
| EGF | 671 . . . 702 | 15/47 (32%) | 1.1e−09 |
| | | 30/47 (64%) | |
| EGF | 709 . . . 740 | 13/47 (28%) | 0.00072 |
| | | 23/47 (49%) | |
| EGF | 748 . . . 779 | 17/47 (36%) | 3.1e−09 |
| | | 27/47 (57%) | |
| EGF | 786 . . . 817 | 17/47 (36%) | 3.5e−07 |
| | | 28/47 (60%) | |
| EGF | 824 . . . 855 | 16/47 (34%) | 1.7e−05 |
| | | 25/47 (53%) | |
| vwc | 863 . . . 917 | 18/84 (21%) | 0.055 |
| | | 33/84 (39%) | |

### Example 18

The NOV18 clone was analyzed, and the nucleotide and encoded polypeptide sequences are shown in Table 18A.

TABLE 18A
NOV18 Sequence Analysis

| | SEQ ID NO: 59 | 587 bp | |
|---|---|---|---|
| NOV18a. | GGAGCTTGCTGACCATCCCTGGGAGCTTTAATGTTTACTTCTATCTTGCAGAGTTTTTCA | | |
| CG139363-01 | | | |
| DNA Sequence | CTGAACTTCACCCTGCCGGCGAACACAGTAAGTACAGCAGCCCCCATTCAGACATCTGGT | | |
| | AAGGGCCACTGTGGGCCCTCTCTTGGATTAGCGGCGGGCATACCATTCCTGGTGGCCACA | | |
| | GCCCTGCTGGTGGCTTTACTATTTACTTTGATTCACCGAAGAAGAAGCAGCATTGAGGCC | | |
| | ATGGAGGTGATTAGTCCATCTTGTATGAAAGAATTCTCTGCTGTAGTTTTTAAAAAACCT | | |
| | ATTTGTTTCCTTAAGAATCCTAGGAGATCACCCACACATGAGAAGAATACGATGGGAGCA | | |
| | CAAGAGGCCCACATATATGTGAAGACTGTAGCAGGAAGCGAGGAACCTGTGCATGACCGT | | |
| | TACCGTCCTACTATAGAAATGGAAAGAAGGAGGGGATTGTGGTGGCTTGTGCCCAGACTG | | |
| | AGCCTGGAATTGATGCAGCTCAGTCAAGGAGCAGCAGACCTGGCACTGGAACAGGGTTGA | | |
| | AAACCCAGGGTTTTGTACTTGGAGAGGAAAGATGCCAAGCTGCTTCT | | |
| | ORF Start: ATG at 31 | | ORF Stop: TGA at 538 |
| | SEQ ID NO: 60 | 169 aa | MW at 18578.4kD |
| NOV18a, | MFTSILQSFSLNFTLPANTVSTAAPIQTSGKGDCGPSLGLAAGIPLLVATALLVALLFTL | | |
| CG139363-01 | | | |
| Protein Sequence | IHRRRSSIEAMEVISPSCMKEFSAVVFKKPICFLKNPRRSPTHEKNTMGAQEAHIYVKTV | | |
| | AGSEEPVHDRYRPTIEMERRRGLWWLVPRLSLELMQLSQGAADLALEQG | | |
| | SEQ ID NO: 61 | 528 bp | |
| NOV18b, | GGGAGCTTTAATGTTTACTTCTATCTTGCAGAGTTTTTCACTGAACTTCACCCTGCCGGC | | |
| CG139363-02 | | | |
| DNA Sequence | GAACACAACGTCCTCTCCTGTCACAGGTGGGAAAGAAACGGACTGTGGGCCCTCTCTTGG | | |
| | ATTAGCGGCGGCCATACCATTGCTGGTGGCCACAGCCCTGCTGGTGGCTTTACTATTTAC | | |
| | TTTGATTCACCGAAGAAGAAGCAGCATTGAGGCCATGGAGGAAAGTGACAGACCATGTGA | | |
| | AATTTCAGAAATTGATGACAATCCCAAGATATCTGAGAATCCTAGGAGATCACCCACACA | | |
| | TGAGAAGAATACGATGGGAGCACAAGAGGCCCACATATATGTGAAGACTGTAGCAGGAAG | | |
| | CGAGGAACCTGTGCATGACCGTTACCGTCCTACTATAGAAATGGAAAGAAGGAGGGGATT | | |
| | GTGGTGGCTTGTGCCCAGACTGAGCCTGGAATGATGCAGCTCAGTCAAGGAGCAGCAGAC | | |
| | CTGGCACTGGAACAGGGTTGAAAACCCAGGGTTTTGTACTTGGAGAGG | | |
| | ORF Start: ATG at 11 | | ORF Stop: TGA at 452 |
| | SEQ ID NO: 62 | 147 aa | MW at 16372.4kD |

| | |
|---|---|
| NOV18b, | MFTSILQSFSLNFTLPANTTSSPVTGGKETDCGPSLGLAAGIPLLVATALLVALLFTLIH |
| CG139363-02 | |
| Protein Sequence | RRRSSIEAMEESDRPCEISEIDDNPKISENPRRSPTHEKNTMGAQEAHIYVKTVAGSEEP |
| | VHDRYRPTIEMERRRGLWWLVPRLSLE |

Sequence comparison of the above protein sequences yields the following sequence relationships shown in Table 18B.

TABLE 18B
Comparison of NOV18a against NOV18b.

| | NOV18a Residues/ | Identities/Similarities |
|---|---|---|
| Protein Sequence | Match Residues | for the Matched Region |
| NOV18b | 1 . . . 153 | 108/153 (70%) |
| | 1 . . . 147 | 114/153 (73%) |

Further analysis of the NOV18a protein yielded the following properties shown in Table 18C.

TABLE 18C
Protein Sequence Properties NOV18a

| | |
|---|---|
| PSort | 0.8569 probability located in mitochondrial inner membrane; |
| analysis: | 0.4456 probability located in mitochondrial intermembrane |
| | space; 0.2847 probability located in mitochondrial matrix |
| | space; 0.2847 probability located in mitochondrial outer |
| | membrane |
| SignalP | Cleavage site between residues 64 and 65 |
| analysis: | |

A search of the NOV18a protein against the Geneseq database, a proprietary database that contains sequences published in patents and patent publication, yielded several homologous proteins shown in Table 18D.

TABLE 18D
Geneseq Results for NOV18a

| | | NOV18a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| ABG23422 | Novel human diagnostic | 8 . . . 153 | 123/153 (80%) | 3e−58 |
| | protein #23413 - Homo | 15 . . . 163 | 127/153 (82%) | |
| | sapiens, 163 aa. | | | |
| | (WO200175067-A2, | | | |
| | 11-OCT-2001] | | | |
| AAM79058 | Human protein SEQ ID NO | 8 . . . 153 | 116/146 (79%) | 1e−56 |
| | 1720 - Homo sapiens, 141 aa. | 2 . . . 141 | 122/146 (83%) | |
| | [WO200157190-A2, | | | |
| | 09-AUG-2001] | | | |
| AAY94922 | Human secreted protein clone | 8 . . . 153 | 115/146 (78%) | 1e−55 |
| | pv6_1 protein sequence SEQ | 2 . . . 141 | 121/146 (82%) | |
| | ID NO: 50 - Homo sapiens, | | | |
| | 141 aa. [WO200009552-A1, | | | |
| | 24-FEB-2000] | | | |
| ABG23423 | Novel human diagnostic | 8 . . . 158 | 115/151 (76%) | 2e−55 |
| | protein #23414 - Homo | 35 . . . 179 | 122/151 (80%) | |
| | sapiens, 209 aa. | | | |
| | [WO200175067-A2, | | | |
| | 11-OCT-2001] | | | |
| AAM80042 | Human protein SEQ ID NO | 8 . . . 141 | 104/134 (77%) | 3e−47 |
| | 3688 - Homo sapiens, 133 aa. | 11 . . . 133 | 109/134 (80%) | |
| | [WO200157190-A2, | | | |
| | 09-AUG-2001] | | | |

In a BLAST search of public sequence datbases, the NOV18a protein was found to have homology to the proteins shown in the BLASTP data in Table 18E.

TABLE 18E
Public BLASTP Results for NOV18a

| | | | Identities/ | |
|---|---|---|---|---|
| | | NOV18a | Similarities | |
| Protein | | Residues/ | for the | |
| Accession | Protein/ | Match | Matched | Expect |
| Number | Organism/Length | Residues | Portion | Value |
| Q96PE5 | Transmembrane | 8 . . . 153 | 116/146 (79%) | 4e−56 |
| | protein | 2 . . . 141 | 122/146 (83%) | |
| | HTMP10 - Homo | | | |
| | sapiens (Human), | | | |
| | 141 aa. | | | |
| Q29102 | Transmembrane | 8 . . . 153 | 104/147 (70%) | 5e−50 |
| | protein sp83.5 - | 2 . . . 142 | 117/147 (78%) | |
| | Sus scrofa (Pig), | | | |
| | 142 aa. | | | |
| P54423 | Cell wall-associated | 91 . . . 167 | 22/77 (28%) | 2.7 |
| | protease precursor | 662 . . . 737 | 39/77 (50%) | |
| | (EC 3.4.21.-) | | | |
| | [Contains: Cell | | | |
| | wall-associated | | | |
| | polypeptides | | | |
| | CWBP23 and | | | |
| | CWBP52] - | | | |
| | Bacillus subtilis, | | | |
| | 894 aa. | | | |
| Q9A7Z7 | Hypothetical protein | 108 . . . 151 | 14/44 (31%) | 3.5 |
| | CC1570 - | 184 . . . 227 | 23/44 (51%) | |
| | Caulobacter | | | |
| | crescentus, 311 aa. | | | |
| Q8S9L6 | AT4g21410/ | 16 . . . 77 | 19/62 (30%) | 3.5 |
| | T6K22_140 - | 265 . . . 326 | 32/62 (50%) | |
| | Arabidopsis | | | |
| | thaliana | | | |
| | (Mouse-ear cress), | | | |
| | 679 aa. | | | |

PFam analysis predicts that the NOV18a protein contains the domains shown in Table 18F.

TABLE 18F
Domain Analysis of NOV18a

| | | | |
|---|---|---|---|
| Pfam Domain | NOV18a Match | Identities/ | Expect Value |
| | Region | Similarities | |
| | | for the Matched | |
| | | Region | |

### Example 19

The NOV19 clone was analyzed, and the nucleotide and encoded polypeptide sequences are shown in Table 19A.

TABLE 19A
NOV19 Sequence Analysis

| | SEQ ID NO: 63 | 471 bp | |
|---|---|---|---|
| NOV19a, | CCACCCTTGCTGCCACTAACATGGAGACTTTGTACCGTGTCCCATTCTTAGTGCTCGAAT | | |
| CG140188-01 | | | |
| DNA Sequence | GTCCCAACCTGAAGCTGAAGAAGCCCCCCTGGCTGCAAGTGCTGTCCGCCATGATTGTGT | | |
| | ATGCTCTGATGGTGGTGTCTTACTTCCTCGTCACTGGAGGAATAATTTATGATGTTATTG | | |
| | TTGAACCTCCAAGCATTGGCTCTATGACTGATGAACACGGGCATCAGAGGCCAGTAGCTT | | |
| | TCTTGGCCTACAGAGTAAATGAACAATGTATTATGGAAGGACTTGCATCCAGCTTCCTGT | | |
| | TTACAATAGGAGGTTTAGGTTTCATATTCCTGGACCGATGGAATGCACCAAATATCCCAA | | |
| | AACTCAATAGATTTCTTCTTCTATTCATTGGATTCGTTTGTGTCCTATTGAGCTTTTTCA | | |
| | TGGCTAGAGTATTCATGAGAATGAAACTGCCGGGCTATCTGATGGGTTAGA | | |
| | ORF Start: ATG at 21 | | ORF Stop: TAG at 468 |
| | SEQ ID NO: 64 | 149 aa | MW at 16975.3kD |

| | |
|---|---|
| NOV19a, | METLYRVPFLVLECPNLKLKKPPWLQVLSAMIVYALMVVSYFLVTGGIIYDVIVEPPSIG |
| CG140188-01 | |
| Protein Sequence | SMTDEHGHQRPVAFLAYRVNEQCIMEGLASSFLFTIGCLGFIFLDRWNAPNIPKLNRFLL |
| | LFIGFVCVLLSFFMARVFMRMKLPGYLMG |

Further analysis of the NOV19a protein yielded the following properties shown in Table 19B.

TABLE 19B
Protein Sequence Properties NOV19a

| | |
|---|---|
| PSort analysis: | 0.6000 probability located in plasma membrane; |
| | 0.4000 probability located in |
| | Golgi body; 0.3000 probability located in |
| | endoplasmic reticulum (membrane); |
| | 0.0300 probability located in mitochondrial |
| | inner membrane |
| SignalP analysis: | Cleavage site between residues 48 and 49 |

A search of the NOV19a protein against the Geneseq database, a proprietary database that contains sequences published in patents and patent publication, yielded several homologous proteins shown in Table 19C.

TABLE 19C
Geneseq Results for NOV19a

| | | NOV19a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAY53631 | A bone marrow | 1 . . . 149 | 137/149 (91%) | 1e−75 |
| | secreted protein | 1 . . . 149 | 142/149 (94%) | |
| | designated | | | |
| | BMS155 - Homo | | | |
| | sapiens, 149 aa | | | |
| | [WO9933979-A2, | | | |
| | 08-JUL-1999] | | | |
| AAY53042 | Human secreted | 1 . . . 149 | 137/149 (91%) | 1e−75 |
| | protein clone | 1 . . . 149 | 142/149 (94%) | |
| | pu282_10 protein | | | |
| | sequence SEQ ID | | | |
| | NO:90 - Homo | | | |
| | sapiens, 149 aa. | | | |
| | [WO9957132-A1, | | | |
| | 11-NOV-1999] | | | |
| AAB12143 | Hydrophobic | 1 . . . 149 | 137/149 (91%) | 1e−75 |
| | domain protein | 1 . . . 149 | 142/149 (94%) | |
| | isolated from | | | |
| | WERI-RB | | | |
| | cells - Homo | | | |
| | sapiens, 149 aa. | | | |
| | [WO200029448- | | | |
| | A2, 25-MAY- | | | |
| | 2000] | | | |
| AAY59670 | Secreted protein | 1 . . . 149 | 137/149 (91%) | 1e−75 |
| | 108-005-5-0-F6- | 1 . . . 149 | 142/149 (94%) | |
| | FL - Homo | | | |
| | sapiens, 149 aa. | | | |
| | [WO9940189-A2, | | | |
| | 12-AUG-1999] | | | |
| AAY60146 | Human | 1 . . . 149 | 137/149 (91%) | 1e−75 |
| | endometrium | 23 . . . 171 | 142/149 (94%) | |
| | tumour EST | | | |
| | encoded protein | | | |
| | 206 - Homo | | | |
| | sapiens, 171 aa. | | | |
| | [DE19817948- | | | |
| | A1, 21-OCT- | | | |
| | 1999] | | | |

In a BLAST search of public sequence datbases, the NOV19a protein was found to have homology to the proteins shown in the BLASTP data in Table 19D.

TABLE 19D
Public BLASTP Results for NOV19a

| | | NOV19a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9NRP0 | DC2 (Hydrophobic | 1 . . . 149 | 137/149 (91%) | 4e−75 |
| | protein HSF-28) | 1 . . . 149 | 142/149 (94%) | |
| | (Hypothetical 16.8 | | | |
| | kDa protein) - | | | |
| | Homo sapiens | | | |
| | (Human), 149 aa. | | | |
| Q9P075 | HSPC307 - Homo | 1 . . . 149 | 137/149 (91%) | 4e−75 |
| | sapiens (Human), | 19 . . . 167 | 142/149 (94%) | |
| | 167 aa (fragment). | | | |
| Q9CPZ2 | 2310008M10Rik | 1 . . . 149 | 136/149 (91%) | 9e−75 |
| | protein (RIKEN | 1 . . . 149 | 142/149 (95%) | |
| | cDNA 2310008M10 | | | |
| | gene) - Mus | | | |
| | musculus (Mouse), | | | |
| | 149 aa. | | | |
| Q9P1R4 | HDCMD45P - | 1 . . . 149 | 136/149 (91%) | 3e−74 |
| | Homo sapiens | 12 . . . 160 | 141/149 (94%) | |
| | (Human), 160 aa | | | |
| | (fragment). | | | |
| Q8TBU1 | Similar to DC2 | 31 . . . 149 | 118/119 (99%) | 4e−63 |
| | protein - Homo | 1 . . . 119 | 118/119 (99%) | |
| | sapiens (Human), | | | |
| | 119 aa. | | | |

PFam analysis predicts that the NOV19a protein contains the domains shown in Table 19E.

TABLE 19E
Domain Analysis of NOV19a

| | | | |
|---|---|---|---|
| Pfam Domain | NOV19a | Identities/ | Expect Value |
| | Match Region | Similarities | |
| | | for the Matched | |
| | | Region | |

### Example 20

The NOV20 clone was analyzed, and the nucleotide and encoded polypeptide sequences are shown in Table 20A.

TABLE 20A
NOV20 Sequence Analysis

| | SEQ ID NO: 65 | 755 bp | |
|---|---|---|---|
| NOV20a, | GGAGCTCTGCTGTCTTCTCAGGGAGACTCTGAGGCTCTGTTGAGAATCATGCTTTGGAGG | | |
| CG140305-01 | | | |
| DNA Sequence | CAGCTCATCTATTGGCAACTGCTGGCTTTGTTTTTCCTCCCTTTTTGCCTGTGTCAAGAT | | |
| | GAATACATGGAGTCTCCACAAACCGGAGGACTACCCCCAGACTGCAGTAAGTGTTGTCAT | | |
| | GGAGACTACAGCTTTCGAGGCTACCAAGGCCCCCCTGGGCCACCGGGCCCTCCTGGCATT | | |
| | CCAGGAAACCATGGAAACAATGGCAACAATGGAGCCACTGGTCATGAAGGAGCCAAAGGT | | |
| | GAGAAGGGCTACCCGGGGATTCCACCAGAACTTCAGATTGCATTCATGGCTTCTCTGGCA | | |
| | ACCCACTTCAGCAATCAGAACAGTGGGATTATCTTCAGCAGTGTTGAGACCAACATTGGA | | |
| | AACTTCTTTGATGTCATCACTGGTAGATTTGGGGCCCCAGTATCAGGTGTGTATTTCTTC | | |
| | ACCTTCAGCATGATGAAGCATGAGGATGTTGAGGAAGTGTATGTGTACCTTATGCACAAT | | |
| | GGCAACACAGTCTTCAGCATGTACAGCTATGAAATGAAGGGCAAATCAGATACATCCAGC | | |
| | AATCATGCTGTGCTCAAGCTAGCCAAAGGGGATGAGGTTTGGCTGCGAATGGGCAATGGC | | |
| | GCTCTCCATGGGGACCACCAACGCTTCTCCACCTTTGCAGGATTCCTGCTCTTTGAAACT | | |
| | AAGTAAATATATGACTAGAATACCTCCACTTTGGG | | |
| | ORF Start: ATG at 49 | | ORF Stop: TAA at 724 |
| | SEQ ID NO:66 | 225 aa | MW at 24836.9kD |
| NOV2Oa, | MLWRQLTYWQLLALFFLPFCLCQDEYMESPQTGGLPPDCSKCCHGDYSFRGYQGPPGPPG | | |
| CG140305-01 | | | |
| Protein Sequence | PPGIPGNHGNNGNNGATGHEGAKGEKGYPGIPPELQIAFMASLATHFSNQNSGIIFSSVE | | |
| | TNIGNFFDVMTGRFGAPVSGVYFFTFSMMKHEDVEEVYVYLMHNGNTVFSMYSYEMKCKS | | |
| | DTSSNHAVLKLAKGDEVWLRMGNGALHGDHQRFSTFAGFLLFETK | | |
| | SEQ ID NO: 67 | 842 bp | |
| NOV20b, | GGAGCTCTGCTGTCTTCTCAGGTACACTCTGAGGCTCTGTTGAGAATCATGCTTTGGAGC | | |
| CG140305-02 | | | |
| DNA Sequence | CAGCTCATCTATTGGCAACTGCTGCCTTTGTTTTTCCTCCCTTTTTGCCTGTGTCAAGAT | | |
| | GAATACATGGAGTCTCCACAAACCGGAGGACTACCCCCAGACTGCAGTAAGTGTTGTCAT | | |
| | GGAGACTACAGCTTTCGAGGCTACCAACGCCCCCCTGGGCCACCGGGCCCTCCTGGCATT | | |
| | CCAGGAAACCATGGAAACAATGGCAACAATGGAGCCACTGGTCATGAAGGAGCCAAAGGT | | |
| | GAGAAGGGCGACAAAGGTGACCTGGGGCCTCGAGGGGAGCGGGGGCAGCATGGCCCCAAA | | |
| | GGACAGAAGGGCTACCCCGGGATTCCACCACAACTTCAGATTGCATTCATGGCTTCTCTG | | |
| | GCAACCCACTTCAGCAATCAGAACAGTGGGATTATCTTCAGCAGTGTTGAGACCAACATT | | |
| | GGAAACTTCTTTGATGTCATGACTGCTAGATTTGGGCCCCCAGTATCACGTGTGTATTTC | | |
| | TTCACCTTCAGCATGATGAAGCATGAGGATGTTGAGGAAGTGTATGTGTACCTTATGCAC | | |
| | AATGGCAACACAGTCTTCAGCATGTACAGCTATGAAATGAAGGGCAAATCAGATACATCC | | |
| | AGCAATCATGCTGTGCTGAAGCTAGCCAAAGGGGATGAGGTTTGGCTGCGAATGGGCAAT | | |
| | GGCGCTCTCCATGGGGACCACCAACGCTTCTCCACCTTTGCAGGATTCCTGCTCTTTGAA | | |
| | ACTAAGTAAATATATGACTACAATAGCTCCACTTTGGGGAAGACTTGTAGCTGAGCTCAT | | |
| | AA | | |
| | ORF Start: ATG at 49 | | ORF Stop: TAA at 787 |
| | SEQ ID NO: 68 | 246 aa | MW at 26994.2kD |

| | |
|---|---|
| NOV2Ob, | MLWRQLIYWQLLALFFLPFCLCQDEYMESPQTCGLPPDCSKCCHGDYSFRGYQCPPCPPG |
| CG140305-02 | |
| Protein Sequence | PPGIPCNHCNNCNNGATGHEGAKGEKGDKGDLGPRCERGQHGPKGEKGYPGIPPELQIAF |
| | MASLATHFSNQNSGIIFSSVETNIGNFFDVMTGRFGAPVSGVYFFTFSMMKHEDVEEVYV |
| | YLMNNGNTVFSMYSYEMKGKSDTSSNHAVLKLAKGDEVWLRMGNGALHGDHQRFSTFAGF |
| | LLFETK |

Sequence comparison of the above protein sequences yields the following sequence relationships shown in Table 20B.

TABLE 20B
Comparison of NOV20a against NOV20b.

| | NOV20a Residues/ | Identities/Similarities |
|---|---|---|
| Protein Sequence | Match Residues | for the Matched Region |
| NOV20b | 1 . . . 225 | 188/246 (76%) |
| | 1 . . . 246 | 188/246 (76%) |

Two polymorphic variants of NOV20a have been identified and are shown in Table 41F. Further analysis of the NOV20a protein yielded the following properties shown in Table 20C.

TABLE 20C
Protein Sequence Properties NOV20a

| | |
|---|---|
| PSort analysis: | 0.7666 probability located in outside; |
| | 0.2383 probability located in microbody |
| | (peroxisome); 0.1000 probability located |
| | in endoplasmic reticulum (membrane); |
| | 0.1000 probability located in endoplasmic |
| | reticulum (lumen) |
| SignalP analysis: | Cleavage site between residues 23 and 24 |

A search of the NOV20a protein against the Geneseq database, a proprietary database that contains sequences published in patents and patent publication, yielded several homologous proteins shown in Table 20D.

TABLE 20D
Geneseq Results for NOV20a

| | | NOV20a | Identities/ | |
|---|---|---|---|---|
| | Protein/ | Residues/ | Similarities for | |
| Geneseq | Organism/Length | Match | the Matched | Expect |
| Identifier | [Patent #, Date] | Residues | Region | Value |
| AAU84371 | Novel human | 1 . . . 225 | 225/246 (91%) | e—134 |
| | secreted or | 1 . . . 246 | 225/246 (91%) | |
| | membrane- | | | |
| | associated protein | | | |
| | #10 - Homo | | | |
| | sapiens, 246 aa. | | | |
| | [WO200204600-A2, | | | |
| | 17-JAN-2002] | | | |
| AAB88447 | Human membrane | 1 . . . 225 | 225/246 (91%) | e—134 |
| | or secretory | 1 . . . 246 | 225/246 (91%) | |
| | protein clone | | | |
| | PSEC0232 - Homo | | | |
| | sapiens, 246 aa. | | | |
| | [EP1067182-A2, | | | |
| | 10-JAN-2001] | | | |
| AAB18909 | A novel polypeptide | 1 . . . 225 | 225/246 (91%) | e—134 |
| | designated | 1 . . . 246 | 225/246 (91%) | |
| | PRO1384 - Homo | | | |
| | sapiens, 246 aa. | | | |
| | [WO200056889-A2, | | | |
| | 28-SEP-2000] | | | |
| AAB29580 | Human adipocyte | 1 . . . 225 | 225/246 (91%) | e—134 |
| | complement related | 1 . . . 246 | 225/246 (91%) | |
| | protein homolog | | | |
| | zacrp3, SEQ ID | | | |
| | NO:2 - Homo | | | |
| | sapiens, 246 | | | |
| | aa. | | | |
| | [WO200063377-A1, | | | |
| | 26-OCT-2000] | | | |
| AAB15548 | Human immune | 1 . . . 225 | 225/246 (91%) | e—134 |
| | system molecule | 1 . . . 246 | 225/246 (91%) | |
| | from Incyte clone | | | |
| | 1890540 - Homo | | | |
| | sapiens, 246 aa. | | | |
| | [WO200060080-A2, | | | |
| | 12-OCT-2000] | | | |

In a BLAST search of public sequence datbases, the NOV20a protein was found to have homology to the proteins shown in the BLASTP data in Table 20E.

TABLE 20E
Public BLASTP Results for NOV20a

| | | NOV20a | Identities/ | |
|---|---|---|---|---|
| Protein | | Residues/ | Similarities for | |
| Accession | Protein/ | Match | the Matched | Expect |
| Number | Organism/Length | Residues | Portion | Value |
| Q9BXJ4 | Complement-clq | 1 . . . 225 | 225/246 (91%) | e−134 |
| | tumor necrosis | 1 . . . 246 | 225/246 (91%) | |
| | factor-related | | | |
| | protein 3 | | | |
| | precursor | | | |
| | (Secretory | | | |
| | protein | | | |
| | CORS26) - | | | |
| | Homo sapiens | | | |
| | (Human), 246 aa. | | | |
| Q9ES30 | Collagenous | 1 . . . 225 | 215/246 (87%) | e−127 |
| | repeat-containing | 1 . . . 246 | 217/246 (87%) | |
| | sequence of | | | |
| | 26 kDa | | | |
| | protein - Mus | | | |
| | musculus | | | |
| | (Mouse), 246 aa. | | | |
| CAC51163 | Sequence 59 | 28 . . . 126 | 98/120 (81%) | 2e−53 |
| | from Patent | 101 . . . 220 | 99/120 (81%) | |
| | WO0149728 - | | | |
| | Homo sapiens | | | |
| | (Human), 223 aa. | | | |
| Q9ESN4 | Gliacolin | 45 . . . 222 | 66/194 (34%) | 1e−22 |
| | precursor - Mus | 64 . . . 253 | 97/194 (49%) | |
| | musculus | | | |
| | (Mouse), 255 aa. | | | |
| Q8TE71 | EEGIL - Homo | 88 . . . 223 | 51/138 (36%) | 3e−22 |
| | sapiens (Human), | 940 . . . | 87/138 (62%) | |
| | 1077 aa. | 1076 | | |

PFam analysis predicts that the NOV20a protein contains the domains shown in Table 20F.

TABLE 20F
Domain Analysis of NOV20a

| | | Identities/ | |
|---|---|---|---|
| | | Similarities | |
| | NOV20a | for the Matched | |
| Pfam Domain | Match Region | Region | Expect Value |
| Collagen | 37 . . . 95 | 23/60 (38%) | 0.00032 |
| | | 37/60 (62%) | |
| Clq | 98 . . . 221 | 45/137 (33%) | 2.3e−17 |
| | | 76/137 (55%) | |

The NOV21 clone was analyzed, and the nucleotide and encoded polypeptide sequences are shown in Table 21A.

TABLE 21A
NOV21 Sequence Analysis

| | SEQ ID NO: 69 | 1725 bp | |
|---|---|---|---|
| NOV21a, | CGGCCGGCGCTGCACACCCGCTGCTCTTGTCCGGGTCTGTGCGGTCCCGAGGGCCCTCCG | | |
| CG140639-01 | | | |
| DNA Sequence | TGCCGCCGGCGCCATGGGCAATTGCCACACGGTGGGGCCCAACGAGGCGCTGGTGGTTTC | | |
| | AGGGGGCTGTTGTGGTTCCGACTATAAACAGTACGTGTTTGGCGGCTGGGCCTGGGCCTG | | |
| | GTGGTGTATCTCCGACACTCAGAGGATTTCCCTAGAGATTATGACGTTGCAGCCCCGCTG | | |
| | CGAGGACGTAGAGACGGCCGAGGGGGTAGCTTTAACTGTGACGGGTGTCGCCCAGGTGAA | | |
| | GATCATGACGGAGAAGGAACTCCTGGCCGTGGCTTGTGAGCAGTTTCTGGGTAAGAATGT | | |
| | GCAGGACATCAAAAACGTCGTCCTGCAGACCCTGGAGGGACATCTGCGCTCCATCCTCGG | | |
| | GACCCTGACAGTGGAGCAGATTTATCAGGACCGGGACCAGTTTGCCAAGCTGGTGCGGGA | | |
| | GGTGGCAGCCCCTGATGTTGGCCGCATGGGCATTGAGATCCTCAGCTTCACCATCAAGGA | | |
| | CGTGTATGACAAAGTGGACTATCTGAGCTCCCTGGGCAAGACGCAGACTGCCGTGGTGCA | | |
| | GAGAGATGCTGACATTGGCGTGGCCGAGGCTGAACGGGACGCAGGCATCCGGGAAGCTGA | | |
| | GTGCAAGAAGGAGATGCTGGATGTGAAGTTCATGGCAGACACCAAGATTGCTGACTCTAA | | |
| | GCGAGCCTTCGAGCTGCAAAAGTCAGCCTTCAGTGAGGAGGTTAACATCAAGACAGCTGA | | |
| | GGCCCAGTTGGCCTATGAGCTGCAGGGGGCCCGTGAACAGCAGAAGATCCGGCAGGAAGA | | |
| | GATTGAGATTGAGGTTGTGCAGCGCAAGAAACAGATTGCCGTGGAGGCACAGGAGATCCT | | |
| | GCGTACGGACAAGGAGCTCATCGCTACAGTGCGCCGGCCTGCCGAGGCCGAGGCCCACCG | | |
| | CATCCAGCAGATTGCCGAGGGTGAAAAGGTGAAGCAGGTCCTCTTGGCACAGGCAGAGGC | | |
| | TGAGAAGATCCGCAAAATCGGGGAGGCGGAAGCGGCAGTCATCGAGGCGATGGGCAAGGC | | |
| | AGAGGCTGAGCGGATGAAGCTCAAGGCAGAAGCCTACCAGAAATACGGGGATGCAGCCAA | | |
| | GATGGCCTTGGTGCTAGAGGCCCTGCCCCAGATTGCTGCCAAAATCGCTGCCCCACTTAC | | |
| | CAAGGTCGATGAGATTGTGGTCCTCAGTGGAGACAACAGTAAGGTCACATCAGAAGTGAA | | |
| | CCGACTGCTGGCCGAGCTGCCTGCCTCTGTGCATGCCCTCACAGGCGTGGACCTGTCTAA | | |
| | GATACCCCTGATCAAGAAGGCCACTGGTGTGCAGCTGTGAGGCTCCTGCAGGCCCACTCT | | |
| | CTTCAGCACCCACCCGGCCCTCCCTCCAGCACCCCTTTTAATCCCACAGAACAACGGGAA | | |
| | CGTTACTGACTCTCGTGCCTTATCTCCAAGGGACCACAAGTGCTGCGTGTTCAGGCCATC | | |
| | TCTGGCTGTCTTCCTGTCTCTCCTGTCTGTCCACCTCCTCCTCTTCCTCTCCTTTACCCC | | |
| | ACTTTCACTGCCACTTTCATCAGGTTTGTGTCTCATCTCCCTGCGTGTCTTTTCCTTTGT | | |
| | CTGTCTTTTTCTTTCCCCCATGCACATCATGTAGATTAAGCTGAAGATGTTTATTACAAT | | |
| | CACTCTCTGTGGGGGGTGGCCCTGCTGCTCCTCAGAATCCTGGTG | | |
| | ORF Start: ATG at 74 | | ORF Stop: TGA at 1358 |
| | SEQ ID NO: 70 | 428 aa | MW at 47063.7kD |
| NOV21a, | MGNCHTVGPNEALVVSGGCCGSDYKQYVFGGWAWAWWCISDTQRISLEIMTLQPRCEDVE | | |
| CG140639-O1 | | | |
| Protein Sequence | TAEGVALTVTGVAQVKIMTEKELLAVACEQFLGKNVQDIKNVVLQTLEGHLRSILGTLTV | | |
| | EQIYQDRDQFAKLVREVAAPDVCRMCIETLSPTIKDVYDKVDYLSSLGKTQTAVVQRDAD | | |
| | IGVAEAERDAGIREAECKKEMLDVKFMADTKIADSKRAFELQKSAFSEEVNTKTAEAQLA | | |
| | YELQGAREQQKIRQEEIEIEVVQRKKQIAVEAQEILRTDKELIATVRRPAEAEAHRIQQI | | |
| | AEGEKVKQVLLAQAEAEKIRKIGEAEAAVIEAMGKAEAERMKLKAEAYQKYGDAAKMALV | | |
| | LEALPQIAAKIAAPLTKVDEIVVLSGDNSKVTSEVNRLLAELPASVHALTGVDLSKIPLI | | |
| | KKATGVQV | | |
| | SEQ ID NO: 71 | 1389 bp | |
| NOV21b, | CTGCTGTTGTCCGGGTCTGTGCCGTCCCGAGCCCCCTCCCTGCCGCCGGCGCCATGGGCA | | |
| CG140639-02 | | | |
| DNA Sequence | ATTGCCACACGGTGCGGCCCAACGAGGCGCTGGTGGTTTCAGGGGGCTCTTGTGGTTCCG | | |
| | ACTATAAACAGTACGTGTTTGGCCGCTCGGCCTGCGCCTGGTGGTGTATCTCCGACACTC | | |
| | AGAGGATTTCCCTAGAGATTATGACGTTGCAGCCCCGCTGCGAGGACGTAGAGACGGCCG | | |
| | AGGGGGTAGCTTTAACTGTGACGGGTGTCGCCCAGGTGAAGATCATGACGGAGAAGGAAC | | |
| | TCCTGCAGACCCTCGAGGGACATCTGCGCTCCATCCTCGGCACCCTGACAGTGGAGCAGA | | |
| | TTTATCAGGACCGGGACCAGTTTGCCAAGCTGGTGCGCGAGGTGGCAGCCCCTGATGTTG | | |
| | GCCGCATGGGCATTGAGATCCTCAGCTTCACCATCAAGGACGTGTATGACAAAGTGGACT | | |
| | ATCTGAGCTCCCTGCGCAAGACGCAGACTGCCGTGGTGCAGAGAGATGCTGACATTGGCG | | |
| | TGGCCGAGGCTGAACGGGACGCAGGCATCCGGGAAGCTGAGTGCAAGAAGGAGATGCTGG | | |
| | ATGTGAAGTTCATGGCAGACACCAAGATTGCTGACTCTAAGCGAGCCTTCGAGCTGCAAA | | |
| | AGTCAGCCTTCAGTGAGGAGGTTAACATCAAGACAGCTGAGGCCCACTTGGCCTATGAGC | | |
| | TGCAGGGGGCCCGTGAACACCAGAAGATCCGGCAGGAAGAGATTGAGATTGACCTTGTGC | | |
| | AGCGCAAGAAACAGATTGCCGTGGAGGCACAGGAGATCCTGCGTACGGACAAGGAGCTCA | | |
| | TCGCTACAGTGCGCCGGCCTGCCGAGGCCGACGCCCACCGCATCCAGCAGATTGCCGAGG | | |
| | GTGAAAAGGTGAAGCAGGTCCTCTTGGCACAGGCAGAGGCTGAGAAGATCCGCAAAATCG | | |
| | GGGAGGCGGAAGCGGCAGTCATCGAGGCGATGGGCAAGGCAGAGGCTGAGCGGATGAAGC | | |
| | TCAAGGCAGAAGCCTACCAGAAATACGGGGATGCAGCCAAGATGGCCTTGGTCCTAGAGG | | |
| | CCCTGCCCCAGATTGCTGCCAAAATCGCTGCCCCACTTACCAAGGTCGATGACATTGTGG | | |
| | TCCTCAGTGGAGACAACAGTAAGGTCACATCAGAAGTGAACCGACTGCTCGCCGAGCTGC | | |
| | CTGCCTCTGTGCATGCCCCCACAGGCGTGGACCTGTCTAAGATACCCCTGATCAAGAAGG | | |
| | CCACTGGTCTGCAGGTGTGAGGCTCCTGCAGGCCCACTCTCTTCAGCAGCCACCCGGCCC | | |
| | TCCCTCCAG | | |
| | ORF Start: ATG at 54 | | ORF Stop: TGA at 1338 |
| | SEQ ID NO: 72 | 428 aa | MW at 47047.6kD |

| | |
|---|---|
| NOV21b, | MGNCHTVGPNEALVVSGGCCGSDYKQYVFGGWAWAWWCISDTQRISLEIMTLQPRCEDVE |
| CG140639-02 | |
| Protein Sequence | TAEGVALTVTGVAQVKIMTEKELLAVACEQFLGKNVQDIKNVVLQTLEGHLRSILGTLTV |
| | EQIYQDRDQFAKLVREVAAPDVGRMGIEILSFTIKDVYDKVDYLSSLGKTQTAVVQRDAD |
| | IGVAEAERDAGIREAECKKEMLDVKFMADTKIADSKRAFELQKSAFSEEVNIKTAEAQLA |
| | YELQGAREQQKIRQEEIEIEVVQRKKQIAVEAQEILRTDKELIATVRRPAEAEAHRIQQI |
| | AEGEKVKQVLLAQAEAEKIRKIGEAEAAVIEAMGKAEAERMKLKAEAYQKYGDAAKMALV |
| | LEALPQIAAKIAAPLTKVDEIVVLSGDNSKVTSEVNRLLAELPASVHAPTGVDLSKIPLI |
| | KKATGVQV |

Sequence comparison of the above protein sequences yields the following sequence relationships shown in Table 21B.

TABLE 21B
Comparison of NOV21a against NOV21b.

| | | Identities/ |
|---|---|---|
| | | Similarities |
| Protein | NOV21a Residues/ | for the Matched |
| Sequence | Match Residues | Region |
| NOV21b | 1 . . . 428 | 407/428 (95%) |
| | 1 . . . 428 | 407/428 (95%) |

Further analysis of the NOV21a protein yielded the following properties shown in Table 21C.

TABLE 21C
Protein Sequence Properties NOV21a

| | |
|---|---|
| PSort | 0.4500 probability located in cytoplasm; |
| analysis: | 0.3000 probability located in microbody |
| | (peroxisome); 0.1000 probability located |
| | in mitochondrial matrix space; 0.1000 |
| | probability located in lysosome (lumen) |
| SignalP | No Known Signal Sequence Predicted |
| analysis: | |

A search of the NOV21a protein against the Geneseq database, a proprietary database that contains sequences published in patents and patent publication, yielded several homologous proteins shown in Table 21D.

TABLE 21D
Geneseq Results for NOV21a

| | | | Identities/ | |
|---|---|---|---|---|
| | | | Similarities | |
| Geneseq | Protein/Organism/Length | NOV21a Residues/ | for the Matched | Expect |
| Identifier | [Patent #, Date] | Match Residues | Region | Value |
| AAW38288 | Epidermal surface antigen - | 50 . . . 428 | 377/379 (99%) | 0.0 |
| | Homo sapiens, 379 aa. | 1 . . . 379 | 377/379 (99%) | |
| | [US5691460-A, 25 NOV. | | | |
| | 1997] | | | |
| AAR51108 | Human epidermal surface | 50 . . . 326 | 276/277 (99%) | e−148 |
| | antigen - Homo sapiens, 291 | 1 . . . 277 | 276/277 (99%) | |
| | aa. [WO9407906-A, 14 | | | |
| | APR. 1994] | | | |
| ABB69326 | Drosophila melanogaster | 50 . . . 417 | 243/370 (65%) | e−134 |
| | polypeptide SEQ ID NO | 1 . . . 369 | 307/370 (82%) | |
| | 34770 - Drosophila | | | |
| | melanogaster, 378 aa. | | | |
| | [WO200171042-A2, 27 SEP. | | | |
| | 2001] | | | |
| ABB62956 | Drosophila melanogaster | 6 . . . 416 | 202/417 (48%) | e−104 |
| | polypeptide SEQ ID NO | 7 . . . 421 | 301/417 (71%) | |
| | 15660 - Drosophila | | | |
| | melanogaster, 426 aa. | | | |
| | [WO200171042-A2, 27 SEP. | | | |
| | 2001] | | | |
| ABB65943 | Drosophila melanogaster | 6 . . . 416 | 202/421 (47%) | e−102 |
| | polypeptide SEQ ID NO | 7 . . . 425 | 301/421 (70%) | |
| | 24621 - Drosophila | | | |
| | melanogaster, 430 aa. | | | |
| | [WO200171042-A2, 27 SEP. | | | |
| | 2001] | | | |

In a BLAST search of public sequence datbases, the NOV21a protein was found to have homology to the proteins shown in the BLASTP data in Table 21E.

TABLE 21E
Public BLASTP Results for NOV21a

| | | NOV21a | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q9Z2S9 | Flotillin-2 (Reggie-1) (REG- | 1 . . . 428 | 425/428 (99%) | 0.0 |
| | 1) - Rattus norvegicus (Rat), | 1 . . . 428 | 426/428 (99%) | |
| | 428 aa. | | | |
| Q9DC36 | Adult male lung cDNA, | 1 . . . 428 | 424/428 (99%) | 0.0 |
| | RIKEN full-length enriched | 1 . . . 428 | 425/428 (99%) | |
| | library, clone: 1200003P16, | | | |
| | full insert sequence - Mus | | | |
| | musculus (Mouse), 428 aa. | | | |
| Q9BTI6 | Similar to flotillin 2 - Homo | 1 . . . 375 | 374/375 (99%) | 0.0 |
| | sapiens (Human), 385 aa. | 1 . . . 375 | 374/375 (99%) | |
| Q14254 | Flotillin-2 (Epidermal surface | 50 . . . 428 | 379/379 (100%) | 0.0 |
| | antigen) (ESA) - Homo | 1 . . . 379 | 379/379 (100%) | |
| | sapiens (Human), 379 aa. | | | |
| Q60634 | Flotillin-2 (Epidermal surface | 50 . . . 428 | 376/379 (99%) | 0.0 |
| | antigen) (ESA) - Mus | 1 . . . 379 | 377/379 (99%) | |
| | musculus (Mouse), 379 aa. | | | |

PFam analysis predicts that the NOV21a protein contains the domains shown in Table 21F.

TABLE 21F
Domain Analysis of NOV21a

| | | Identities/ | |
|---|---|---|---|
| | | Similarities | |
| Pfam | NOV21a Match | for the Matched | Expect |
| Domain | Region | Region | Value |
| Band_7 | 12 . . . 190 | 37/215 (17%) | 0.28 |
| | | 99/215 (46%) | |

### Example 22

The NOV22 clone was analyzed, and the nucleotide and encoded polypeptide sequences are shown in Table 22A.

TABLE 22A
NOV22 Sequence Analysis

| | SEQ ID NO: 73 | 1201 bp | |
|---|---|---|---|
| NOV22a, | CCGCGGACTGCAGCGACCGCGCCGCCGCTGAGGGAGGCGCCCCACCATGCCGCGGGCCCC | | |
| CG140843-O1 | | | |
| DNA Sequence | GGCGCCGCTGTACGCCTGCCTCCTGGGGCTCTGCGCGCTCCTGCCCCCCCTCCCAGGTCT | | |
| | CAACATATGCACTAGTGGAAGTGCCACCTCATGTGAAGAATGTCTGCTAATCCACCCAAA | | |
| | ATGTGCCTGGTGCTCCAAAGAGGACTTCGGAAGCCCACGCTCCATCACCTCTCGCTCTGA | | |
| | TCTGACGCCAAACCTTGTCAAAAATGGCTGTCGAGGTGAGATAGAGACCCCAGCCAGCAC | | |
| | CTTCCATGTCCTGAGGACCCTGCCCCTCACCAGCAAGGGTTCGGGCTCTGCAGGCTGGGA | | |
| | CGTCATTCAGATGACACCACAGGAGATTGCCGTGAACCTCCGGCCCGGTGACAAGACCAC | | |
| | CTTCCAGCTACAGCTTCCCCAGGTGCAGGACTATCCTGTGGACCTGTACTACCTGATGGA | | |
| | CCTCTCCCTGTCCATGAAGGATGACTTGGACAATATCCGGAGCCTGGGCACCAAACTCGC | | |
| | GGAGGAGATGAGGAAGCTCACCAGCAACTTCCGGTTGGGATTTGGGTCTTTTGTTGATAA | | |
| | GGACATCTCTCCTTTCTCCTACACGGCACCGAGGTACCAGACCAATCCGTGCATTGGTTA | | |
| | CAAGTTGTTTCCAAATTGCGTCCCCTCCTTTGGGTTCCGCCATCTCCTGCCTCTCACAGA | | |
| | CAGAGTGGACAGCTTCAATGAGGAAGTTCGGAAACAGAGGGTGTCCCGGAACCGAGATGC | | |
| | CCCTGAGGGGGGCTTTGATGCAGTACTCCAGGCAGCCGTCTGCAAGGTAACTTTCCTTTC | | |
| | TGGTCCTGTCCCTGCATCGCGAGGTCAAGGTAGAGAGCGTCAGTGCGTGTTCGTACTTCC | | |
| | TGCAGGAGTCTTTGAGTGCCCCAGCATGTGGCTCCTGACCACTCTGAAGTCAGAGGGTGA | | |
| | GCTCAGTGGAACTTCTGGGAAATCTACAGCAGTCAAATCAGCCGGAGCTCGGGAATGGAT | | |
| | TGGGCTGGTCTGTGTCTCTGTGTCAGGGTGTGGTTGTGTGCAATGGAGTACTGTCTGCTA | | |
| | GAAGACAGCTGTCTGCATTTATACATTGGCTTTTTGGTTTATTTTCAGGGGGAAAAAGTA | | |
| | AAGGTCAAGTCATAGGCATAGAAGCTTGTAGAGCTTTCTGGACCAATTTTGGCAAACCTT | | |
| | A | | |
| | ORF Start: ATG at 47 | | ORF Stop: TAG at 1079 |
| | SEQ ID NO: 74 | 344 aa | MW at 37466.6kD |

| | |
|---|---|
| NOV22a, | MPRAPAPLYACLLGLCALLPRLAGLNICTSGSATSCEECLLIHPKCAWCSKEDFGSPRSI |
| CG140843-O1 | |
| Protein Sequence | TSRCDLRANLVKNGCGGEIESPASSGHVLRSLPLSSKGSGSAGWDVIQMTPQEIAVNLRP |
| | GDKTTFQLQVRQVEDYPVDLYYLMDLSLSMKDDLDNIRSLGTKLAEEMRKLTSNFRLGFG |
| | SFVDKDISPFSYTAPRYQTNPCIGYKLFPNCVPSFGFRHLLPLTDRVDSFNEEVRKQRVS |
| | RNRDAPEGGFDAVLQAAVCKVTFLSGPVPAWGGQGRERQWVLVLPAGVFECPSMWLLTTL |
| | KSEGELSGTSGKSTAVKSAGAREWIGLVCVSVSGCGCVQWSTVC |

One polymorphic variant of NOV22a has been identified and is shown in Table 41G. Further analysis of the NOV22a protein yielded the following properties shown in Table 22B.

TABLE 22B
Protein Sequence Properties NOV22a

| | |
|---|---|
| PSort | 0.4849 probability located in outside; |
| analysis: | 0.1000 probability located in endoplasmic |
| | reticulum (membrane); 0.1000 probability |
| | located in endoplasmic reticulum (lumen); |
| | 0.1000 probability located in lysosome |
| | (lumen) |
| SignalP | Cleavage site between residues 25 and 26 |
| analysis: | |

A search of the NOV22a protein against the Geneseq database, a proprietary database that contains sequences published in patents and patent publication, yielded several homologous proteins shown in Table 22C.

TABLE 22C
Geneseq Results for NOV22a

| | | NOV22a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAU76337 | Human anti-dual integrin | 1 . . . 260 | 260/260 (100%) | e−153 |
| | protein #3 - Homo sapiens, | 1 . . . 260 | 260/260 (100%) | |
| | 799 aa. [WO200212501-A2, | | | |
| | 14 FEB. 2002] | | | |
| AAW02194 | Human integrin beta subunit | 1 . . . 260 | 260/260 (100%) | e−153 |
| | protein, beta-5 - Homo | 1 . . . 260 | 260/260 (100%) | |
| | sapiens, 799 aa. | | | |
| | [US5527679-A, 18 JUN. | | | |
| | 1996] | | | |
| AAW13573 | Mouse beta-3 integrin - Mus | 5 . . . 259 | 149/260 (57%) | 5e−77 |
| | sp, 787 aa. [WO9708316- | 6 . . . 257 | 186/260 (71%) | |
| | A1, 06 MAR. 1997] | | | |
| AAW13574 | Mouse beta-3 integrin | 5 . . . 259 | 149/260 (57%) | 5e−77 |
| | (truncated) - Mus sp, 720 aa. | 6 . . . 257 | 186/260 (71%) | |
| | [WO9708316-A1, 06 MAR. | | | |
| | 1997] | | | |
| AAU76336 | Human anti-dual integrin | 5 . . . 259 | 149/260 (57%) | 1e−76 |
| | protein #2 - Homo sapiens, | 7 . . . 258 | 184/260 (70%) | |
| | 788 aa. [WO200212501-A2, | | | |
| | 14 FEB. 2002] | | | |

In a BLAST search of public sequence datbases, the NOV22a protein was found to have homology to the proteins shown in the BLASTP data in Table 22D.

TABLE 22D
Public BLASTP Results for NOV22a

| | | NOV22a | Identities/ | |
|---|---|---|---|---|
| Protein | | Residues/ | Similarities | |
| Accession | | Match | for the Matched | Expect |
| Number | Protein/Organism/Length | Residues | Portion | Value |
| A38308 | integrin beta-5 chain | 1 . . . 260 | 260/260 (100%) | e−153 |
| | precursor - human, 799 aa. | 1 . . . 260 | 260/260 (100%) | |
| P18084 | Integrin beta-5 precursor - | 1 . . . 260 | 260/260 (100%) | e−153 |
| | Homo sapiens (Human), 799 aa. | 1 . . . 260 | 260/260 (100%) | |
| O70309 | Integrin beta-5 precursor - | 1 . . . 260 | 241/260 (92%) | e−141 |
| | Mus musculus (Mouse), 798 aa. | 1 . . . 260 | 252/260 (96%) | |
| Q8SQB9 | Integrin beta 5 subunit | 1 . . . 260 | 235/260 (90%) | e−137 |
| | precursor protein - Bos taurus | 1 . . . 260 | 246/260 (94%) | |
| | (Bovine), 800 aa. | | | |
| Q9GK49 | Integrin beta-5 subunit - Bos | 11 . . . 260 | 225/250 (90%) | e−131 |
| | taurus (Bovine), 791 aa | 2 . . . 251 | 235/250 (94%) | |
| | (fragment). | | | |

PFam analysis predicts that the NOV22a protein contains the domains shown in Table 22E.

TABLE 22E
Domain Analysis of NOV22a

| | | Identities/ | |
|---|---|---|---|
| | | Similarities | |
| Pfam | NOV22a Match | for the Matched | Expect |
| Domain | Region | Region | Value |
| integrin_B | 35 . . . 260 | 142/230 (62%) | 1.4e−185 |
| | | 225/230 (98%) | |

### Example 23

The NOV23 clone was analyzed, and the nucleotide and encoded polypeptide sequences are shown in Table 23A.

TABLE 23A
NOV23 Sequence Analysis

| | SEQ ID NO: 75 | 1272 bp | |
|---|---|---|---|
| NOV23a, | CCTAGGCCACGTGCTGCTGGGTCTCAGTCCTCCACTTCCCGTGTCCTCTGGAAGTTGTCA | | |
| CG141540-01 | | | |
| DNA Sequence | GGAGCAATGTTGCGCTTGTACGTGTTGGTAATGGGAGTTTCTGCCTCCACCCTTCAGCCT | | |
| | GCGGCACACACAGGGGCTGCCAGAAGCTGCCGGTTTCGTGGGAGGCATTACAAGCGGGAG | | |
| | TTCAGGCTGGAAGGGGAGCCTGTAGCCCTGAGGTGCCCCCAGGTGCCCTACTGGTTGTGG | | |
| | CCAGGAGAAGAAGAGACACGGATGTGGGCCCAGGACGGTGCTCTGTGGCTTCTGCCAGCC | | |
| | TTGCAGGAGGACTCTGGCACCTACGTCTGCACTACTAGAAATGCTTCTTACTGTGACAAA | | |
| | ATGTCCATTGAGCTCAGAGTTTTTGAGAATACAGATGCTTTCCTCCCGTTCATCTCATAC | | |
| | CCGCAAATTTTAACCTTGTCAACCTCTGGGGTATTAGTATGCCCTGACCTGAGTGAATTC | | |
| | ACCCGTGACAAAACTGACGTGAAGATTCAATGGTACAAGGATTCTCTTCTTTTGGATAAA | | |
| | GACAATGAGAAATTTCTAAGTGTGAGGGCGACCACTCACTTACTCGTACACGATCTGGCC | | |
| | CTGGAAGATGCTGGCTATTACCGCTGTGTCCTGACATTTGCCCATGAAGGCCAGCAATAC | | |
| | AACATCACTAGGAGTATTGAGCTACGCATCAAGAGGTCAAGACTGACAATCCCGTGTAAG | | |
| | GTGTTTCTGGGAACCGGCACACCCTTAACCACCATGCTGTGGTGGACGGCCAATGACACC | | |
| | CACATAGAGAGCGCCTACCCGGGAGGCCGCGTGACCGAGGGGCCACGCCAGGAATATTCA | | |
| | GAAAATAATGAGAACTACATTGAAGTGCCATTGATTTTTGATCCTGTCACAAGAGAGGAT | | |
| | TTGCACATGGATTTTAAATGTGTTGTCCATAATACCCTGAGTTTTCAGACACTACGCACC | | |
| | ACAGTCAAGGAAGCCTCCTCCACGTTCTCCTGGGGCATTGTGCTGGCCCCACTTTCACTG | | |
| | GCCTTCTTGGTTTTGGGGGGAATATGGATGCACAGACGGTGCAAACACAGAACTGGAAAA | | |
| | GCAGATGGTCTGACTGTGCTATGGCCTCATCATCAAGACTTTCAATCCTATCCCAAGTGA | | |
| | AATAAATGGAATGAAATAATTCAAACACAAACTCCGTACGTCTTCTCTTATGGAAGTGGC | | |
| | TGTGTCTTTTTG | | |
| | ORF Start: ATG at 67 | | ORF Stop: TGA at 1198 |
| | SEQ ID NO: 76 | 377 aa | MW at 43181.9kD |
| NOV23a, | MLRLYVLVMGVSASTLQPAAHTGAARSCRFRGRHYKREFRLEGEPVALRCPQVPYWLWAS | | |
| CG141540-01 | | | |
| Protein Sequence | VSPRINLTWHKNDSARTVPGEEETRMWAQDGALWLLPALQEDSGTYVCTTRNASYCDKMS | | |
| | IELRVFENTDAFLPFISYPQILTLSTSGVLVCPDLSEFTRDKTDVKIQWYKDSLLLDKDN | | |
| | LGTGTPLTTMLWWTANDTHIESAYPGGRVTEGPRQEYSENNENYIEVPLTFDPVTREDLH | | |
| | MDFKCVVHNTLSFQTLRTTVKEASSTFSWGIVLAPLSLAFLVLGGIWMHRRCKHRTGKAD | | |
| | GLTVLWPHHQDFQSYPK | | |
| | SEQ ID NO: 77 | 1286 bp | |
| NOV23b, | GCCACGTGCTGCTGGGTCTCAGTCCTCCACTTCCCGTGTCCTCTGGAAGTTGTCAGGAGC | | |
| CG141540-02 | | | |
| DNA Sequence | AATGTTGCGCTTGTACGTGTTGGTAATGCGACTTTCTGCCTTCACCCTTCACCCTCCGCC | | |
| | ACACACAGGGGCTGCCAGAAGCTGCCGGTTTCGTGGGAGGCATTACAAGCGGGAGTTCAG | | |
| | GCTGGAAGGGGAGCCTGTAGCCCTGAGGTGCCCCCAGGTGCCCTACTGGTTGTGGGCCTC | | |
| | TGTCAGCCCCCGCATCAACCTGACATGGCATAAAAATGACTCTGCTAGGACGGTCCCAGG | | |
| | AGAACAAGACACACGGATGTGGGCCCAGGACGGTGCTCTGTGGCTTCTGCCACCCTTGCA | | |
| | GGAGGACTCTGGCACCTACGTCTGCACTACTAGAAATGCTTCTTACTGTGACAAAATGTC | | |
| | CATTGAGCTCACAGTTTTTGAGAATACACATGCTTTCCTGCCGTTCATCTCATACCCGCA | | |
| | AATTTTAACCTTGTCAACCTCTGGGGTATTAGTATGCCCTGACCTGAGTGAATTCACCCG | | |
| | TGACAAAACTGACGTGAAGATTCAATGGTACAAGGATTCTCTTCTTTTGGATAAAGACAA | | |
| | TGAGAAATTTCTAAGTGTGAGGGGGACCACTCACTTACTCGTACACGATGTGGCCCTGGA | | |
| | AGATGCTGGCTATTACCGCTGTGTCCTGACATTTGCCCATGAAGGCCAGCAATACAACAT | | |
| | CACTAGGAGTATTGAGCTACGCATCAAGAAAAAAAAAGAAGAGACCATTCCTGTGATCAT | | |
| | TTCCCCCCTCAAGACCATATCAGCTTCTCTGGGGTCAAGACTGACAATCCCGTGTAAGGT | | |
| | GTTTCTGGGAACCGGCACACCCTTAACCACCATGCTGTGGTGGACGGCCAATGACACCCA | | |
| | CATAGAGAGCGCCTACCCGGGAGGCCGCGTGACCGAGGGGCCACGCCAGGAATATTCAGA | | |
| | AAATAATGAGAACTACATTGAAGTGCCATTGATTTTTGATCCTGTCACAAGAGAGGATTT | | |
| | GCACATGGATTTTAAATGTGTTGTCCATAATACCCTGAGTTTTCAGACACTACGCACCAC | | |
| | AGTCAACGAAGCCTCCTCCACGTTCTCCTCGGGCATTCTGCTGGCCCCACTTTCACTGGC | | |
| | CTTCTTGGTTTTGGGGGGAATATGGATGCACAGACGGTGCAAACACAGAACTGGAAAAGC | | |
| | AGATGGTCTGACTGTGCTATGGCCTCATCATCAAGACTTTCAATCCTATCCCAAGTGAAA | | |
| | TAAATGGAATGAAATAATTCAAACAC | | |
| | ORF Start: ATG at 62 | | ORF Stop: TGA at 1256 |
| | SEQ ID NO: 78 | 398 aa | MW at 45420.6kD |

| | |
|---|---|
| NOV23b | MLRLYVLVMGVSAFTLQPAAHTGAARSCRFRGRHYKREFRLEGEPVALRCPQVPYWLWAS |
| CG141540-02 | |
| Protein Sequence | VSPRINLTWHKNDSARTVPGEEETRMWAQDGALWLLPALQEDSGTYVCTTRNASYCDKMS |
| | IELRVFENTDAFLPFISYPQILTLSTSGVLVCPDLSEFTRDKTDVKIQWYKDSLLLDKDN |
| | EKFLSVRGTTHLLVHDVALEDAGYYRCVLTFAHEGQQYNITRSIELRIKKKKEETIPVII |
| | SPLKTISASLGSRLTIPCKVFLGTGTPLTTMLWWTANDTHIESAYPGGRVTEGPRQEYSE |
| | NNENYIEVPLIFDPVTREDLHMDFKCVVHNTLSFQTLRTTVKEASSTFSWGIVLAPLSLA |
| | FLVLGGIWMHRRCKHRTGKADGLTVLWPHHQDFQSYPK |

Sequence comparison of the above protein sequences yields the following sequence relationships shown in Table 23B.

TABLE 23B
Comparison of NOV23a against NOV23b.

| | NOV23a | Identities/ |
|---|---|---|
| | Residues/ | Similarities |
| Protein | Match | for the Matched |
| Sequence | Residues | Region |
| NOV23b | 1 . . . 377 | 375/398 (94%) |
| | 1 . . . 398 | 376/398 (94%) |

Six plymorphic variants of NOV23a have been identified and are shown in Table 41H. Further analysis of the NOV23a protein yielded the following properties shown in Table 23C.

TABLE 23C
Protein Sequence Properties NOV23a

| | |
|---|---|
| PSort | 0.4600 probability located in plasma membrane; |
| analysis: | 0.2676 probability located in microbody |
| | (peroxisome); 0.1000 probability located in |
| | endoplasmic reticulum (membrane); 0.1000 |
| | probability located in endoplasmic reticulum |
| | (lumen) |
| SignalP | Cleavage site between residues 14 and 15 |
| analysis: | |

A search of the NOV23a protein against the Geneseq database, a proprietary database that contains sequences published in patents and patent publication, yielded several homologous proteins shown in Table 23D.

TABLE 23D
Geneseq Results for NOV23a

| | | NOV23a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| ABB08207 | Human type II Interleukin-1 | 1 . . . 377 | 375/398 (94%) | 0.0 |
| | receptor - Homo sapiens, 398 | 1 . . . 398 | 376/398 (94%) | |
| | aa. [WO200187328-A2, 22 | | | |
| | NOV. 2001] | | | |
| AAE16581 | Human interleukin-1 receptor | 1 . . . 377 | 375/398 (94%) | 0.0 |
| | DNAX designation 2 (IL- | 1 . . . 398 | 376/398 (94%) | |
| | 1RD2) protein - Homo | | | |
| | sapiens, 398 aa. | | | |
| | [US6326472-B1, 04 DEC. | | | |
| | 2001] | | | |
| AAU78089 | Human interleukin 1R2 (IL- | 1 . . . 377 | 375/398 (94%) | 0.0 |
| | 1R2) protein sequence - | 1 . . . 398 | 376/398 (94%) | |
| | Homo sapiens, 398 aa. | | | |
| | [WO200211767-A2, 14 | | | |
| | FEB. 2002] | | | |
| AAM24185 | Human EST encoded protein | 1 . . . 377 | 375/398 (94%) | 0.0 |
| | SEQ ID NO: 1710 - Homo | 1 . . . 398 | 376/398 (94%) | |
| | sapiens, 398 aa. | | | |
| | [WO200154477-A2, 02 | | | |
| | AUG. 2001] | | | |
| AAB37792 | Human interleukin-1 | 1 . . . 377 | 375/398 (94%) | 0.0 |
| | receptor, type II precursor - | 1 . . . 398 | 376/398 (94%) | |
| | Homo sapiens, 398 aa. | | | |
| | [WO200064479-A1, 02 | | | |
| | NOV. 2000] | | | |

In a BLAST search of public sequence datbases, the NOV23a protein was found to have homology to the proteins shown in the BLASTP data in Table 23E.

TABLE 23E
Public BLASTP Results for NOV23a

| | | NOV23a | Identities/ | |
|---|---|---|---|---|
| Protein | | Residues/ | Similarities | |
| Accession | | Match | for the Matched | Expect |
| Number | Protein/Organism/Length | Residues | Portion | Value |
| P27930 | Interleukin-1 receptor, type II | 1 . . . 377 | 375/398 (94%) | 0.0 |
| | precursor (IL-1R-2) (IL-1R- | 1 . . . 398 | 376/398 (94%) | |
| | beta) (Antigen CDw121b) - | | | |
| | Homo sapiens (Human), 398 | | | |
| | aa. | | | |
| Q29612 | Interleukin-1 receptor, type II | 1 . . . 372 | 342/393 (87%) | 0.0 |
| | precursor (IL-1R-2) (IL-1R- | 1 . . . 393 | 351/393 (89%) | |
| | beta) - Cercopithecus | | | |
| | aethiops (Green monkey) | | | |
| | (Grivet), 393 aa. | | | |
| AAB05878 | Soluble type II interleukin-1 | 1 . . . 275 | 273/296 (92%) | e−159 |
| | receptor - Homo sapiens | 1 . . . 296 | 274/296 (92%) | |
| | (Human), 296 aa. | | | |
| Q9N2H5 | Interleukin-1 receptor type II | 4 . . . 376 | 258/394 (65%) | e−147 |
| | precursor - Equus caballus | 4 . . . 396 | 297/394 (74%) | |
| | (Horse), 403 aa. | | | |
| P43303 | Interleukin-1 receptor, type II | 1 . . . 376 | 232/411 (56%) | e−127 |
| | precursor (IL-1R-2) - Rattus | 1 . . . 409 | 282/411 (68%) | |
| | norvegicus (Rat), 416 aa. | | | |

PFam analysis predicts that the NOV23a protein contains the domains shown in Table 23F.

TABLE 23F
Domain Analysis of NOV23a

| | | Identities/ | |
|---|---|---|---|
| | | Similarities | |
| Pfam | NOV23a Match | for the Matched | Expect |
| Domain | Region | Region | Value |
| ig | 43 . . . 110 | 13/70 (19%) | 0.00014 |
| | | 46/70 (66%) | |
| ig | 165 . . . 209 | 9/47 (19%) | 0.0011 |
| | | 35/47 (74%) | |
| ig | 230 . . . 307 | 14/78 (18%) | 4.3e−05 |
| | | 56/78 (72%) | |

### Example 24

The NOV24 clone was analyzed, and the nucleotide and encoded polypeptide sequences are shown in Table 24A.

TABLE 24A
NOV24 Sequence Analysis

| | SEQ ID NO: 79 | 4744 bp | |
|---|---|---|---|
| NOV24a, | GCTCGGAACTACACTTCCCGGCACAACGCCGGCGCGCGCACGCGCACCGGCCCCTCAGCC | | |
| CG141580-01 | | | |
| DNA Sequence | ATGGCGACCGTGCTCTCCAGGGCGCTCAACCTGCCGCCGAAGAAGAGCCCAGACCTAGCG | | |
| | GAGTATCATCCACTTACCCAGGCTGACAGTGATGAGAGCGAAGACCATCTGGTGCTTAAC | | |
| | CTGCAGAAGAATGGAGGGGTCAAAAATGGGAAGAGTCCTTTGGGAGAAGCGCCAGAACCC | | |
| | GACTCAGATGCTGAGGTTGCAGAGGCTGCAAAGCCACATCTTTCAGAAGTCACCACGGAG | | |
| | GGCTACCCCTCAGAACCCCTTGGGCCCCTGGAACAGAAGCCGGCCTCCTCCCTGGTGTCA | | |
| | TATCTGCGCACGTCTGTCTTCCTGCTGACTTTGGGGATCTCGATGATCCTGGTGCTCCTG | | |
| | TGTGCTTTCCTGATCCCCTGTCCTCCCAGAGATCTGCACAGCACCTGGAGCCGCCACTTG | | |
| | GGCTCCCAGGGAGGTGGGGACCTGTCTCCATTGGAATTGGCTGATGTGAATGGAGATGGC | | |
| | CTGCGTGATGTGCTTCTCTCCTTTGTGATGTCAACGAACGGCAGTGCACTAGCTGTCTCA | | |
| | AGACCAGCTGCTAATCTTGTATCCCTTTCCGCGATGAATGGCAGCACACTGTGGTCTAGT | | |
| | CTTCTCCCTGACGAGGCTCGAGATATCACATCTTTGGAGCTCATCCCAGGAAGCTTGCCT | | |
| | GAAACCATCTCCCTTGTGACAGGGACACACAAGATGCTCAGCGCATTCAATCCAACGTCA | | |
| | GGGAAAGCCATTTGGACTTTAAACCCAAACTACTTGTCCAACCGTACCTTGGCTCCCCCA | | |
| | GTTGTGGTACTGCCAGACTTGGATGAAGACGCTCTTCGAGACCTTCTGGTTCTGGCCATT | | |
| | GGGGAATTGCAGCCAGATCTGTGCTTTCTGCTGGTGTCTGGCCGCACCGGAAATCCAGTG | | |
| | GTTGTGGTACTGCCAGACTTGGATGAAGACGGTGTTCGAGACCTTGTGGTTCTGGCCATT | | |
| | ATCACCACAAATGGGGCTCTCTACATCCTGTTTGCCTTTGGAAATATACAAGCTGTCGCA | | |
| | CTGCGGGACATTTTTGTTCAGGCCCAAAATCGAGACAGCTCACCACCTTCTCTGCAGATA | | |
| | GAAGAGCCAGAATGGGAAAAGCGAAGATCCATCAACCTGTCTGAGCTCATTCATGTTTAC | | |
| | AGTGATGGTGTTCAACTACTCCAGATGGTGAAGGCACCAGATTCCAACTGCAGCAACCTT | | |
| | CTGATTACAACCAGACAAAGCCTTGTGCTGCTTCGGGGGCAAAATCTGACACCTTACTGG | | |
| | GCATTGAGACTTCAAGGCCTGCGCAGCCAGCCTACTCCTGGATATTTCACTGATGATCAG | | |
| | ACATTAGACTTCCTTCTGCAGATACAGGATGGAGTTGGGATGAAAAACATGATGGTTGTG | | |
| | GATGGTGACTCTGGCTCCATTGTTTGGAGTTACCGTGCTCCGTGTCACATGAAAGAAACG | | |
| | CCAGCCACCTCAGCAGTTACTTCAGACCAGAAGTCTGTCTTCCTCTTCTGGGCCGAAGGG | | |
| | CTGTCAGCTGCATCTCCCAATTCCGATATCATCCTAGGAACTGAGCCGCCCAGCCTTCAC | | |
| | CACCTTTACCTCCTGCATCCTGCGTTCCCCTCCATCCTTCTGGATCTGGCCAACACCACC | | |
| | GGCACAGTGACGGCTTCAGAGGTTGGAATTAACGACCTCTGGAAAGATGCCTTTTATGTT | | |
| | ACCAGGACAACAGGGCCAAGCTCCGAAGGCCATCCAGCAGCCCTGGTGGTCAGCAAGCTT | | |
| | AGTCTACGGTGGGCACTAATGGAGGGCCAGATGGCTCAGCTACAGGAGTCCACCCCCAAA | | |
| | ATTGGCCGTGGGCAGCTGCGAAGATTTCTCTCTAGGATAAAGTTTGTTGAAGCTCCCTAC | | |
| | GAGATCTAATCTGATGGAATCTTCAGTTGCAGAAGAAGTGAACAGAGTGGATACCCTCTC | | |
| | TACTCTCCTGTCACTGTAAAATCAGTTCTATGGAGAGAAGACTTCTTCGTCCTCATTTAC | | |
| | CACCTCCCTGATGGTTGCAAAGCCTTGGGAAGGCATGTTGGAGTCTTTGACGGCAGCATG | | |
| | ATCTATTTGGCTGGGGCATCTTACCTACCTTTTCAGTCCCTGCATTAATCCCCTCTAGGA | | |
| | ACTCTGCGTGGACCGTTTGGAAATGTGAATCTCTTAAGTATTTAATTTTTTTGGTATGTC | | |
| | TAATTTATGAAGTCTTGCTGGGAAAGCCAGTGAAGTCTATGACTAGGAAACATTTTGTTG | | |
| | TACATTGTGCTGTGTGTGTGTATATTTTAGTGTTGTGGTGAACTTATTTTCCAGGTATCT | | |
| | CCTAAGCTTCAGGGATCCAGTTTCTTGTCCTTCTGAAATATATCTGGTTTGTTTGGTCAT | | |
| | TTTGAGACTTCCAGATGCCCTACCTCTGATGTTGAGGGCCACTTATTTCTCTCCTTATTC | | |
| | TTTCCCACCTGTACCTTGGCTACTTCCAAATTGTAGACAGAATGAGAAAGATTTATACTG | | |
| | GAAGACTGAGTTAGCCATCCAAGCATTTTCATCTCTCTTCTTTTATATCCTATTTCCTTA | | |
| | GATTTTCCATCCATGTCTATTAACTGACCACAAGAATAACTATATTCCTATCACAAGGCG | | |
| | AGCAAGAGGATGTACTCTCAGTCACCCATCTCTGACCAAGTCCACATGTTGTGTTATATG | | |
| | TGGCTCTGATGGTTCTGCCAGTCATGATCTTTTTTCTGTCGCGACATCAGAAGTCTATGT | | |
| | TTGCATGCTCTCTTCAACTTAGAGGAGAACTGGAAGTCACGAGCCTTTGATGTCCTTATC | | |
| | CTGCTGTATGTCTTCTCTGCATCTTTTTCTATAGGGCACCCTCCTTACCTCCCCTCACTC | | |
| | TGTTTTCTCTTCTATTCAGGGATATGTTTCTGGACTTTTTCTTCTGCTACTTGAGTCCAG | | |
| | GATGCAACCATTTTGTCCTGCATCTCTTCTTTCCTCTACAGCCTTTGAAGCATTGTATTT | | |
| | TGGGAAAATTCTTCTGTAAATACTATAACTTTTATAAATGGTTAAGTTATTTAGAATTAT | | |
| | CTCCAGTGCTTACTTCTCCCTTCTTCTGTATAAATCTGCTACTTCAATTAAGTTCTCCTC | | |
| | TAAACTTTTACCTCATTGTTTATATAGCAGAAAATTCAATGTTAGCGGATCGAAAACTGC | | |
| | TTCTTGAATAACCTTGATAGGTCATCCCTGAGTGCACCTCAGGTTCTCTCTTTACCTGGC | | |
| | CTTGTATCTTTTTTTTTTTTTTTTTTTTTTTTGACACAGAGTTTTGCTCTTGTCGCCCAG | | |
| | GCTGGAGTGCACTGGCACAATCTCGGCTCACTGCAACCTTCGCCTCCTGGGTTCAAGCGA | | |
| | TTCTCCAGCCTTAGCCTCCCAAGTAGCTGGGACTACAGGTGCCCGCTACCATGCCTGGCT | | |
| | AATTTTTTTTTTTGTATTTTTAGTAGAGACGGGGTTTCACCATGTTGCCCAGGCTGGTCA | | |
| | CGAACTCCTGACCTCAGATAATCCACCTGCTTCTGCCTCCCAAAGTGCTGGGATTACAGG | | |
| | CGTGAGCCACCATGCCCGGCTGGGCTTGTATCTTTTAGCTTGTGTTAGTAAAACGATTCT | | |
| | AGAAAATTATCAAGTCCAGATTCAAAGGGATCTCTGTTAATTACCCACTGACAGGCATTA | | |
| | TGACCTAACAGCAGGTTGGTAGCAGTAGATCCAAGCATGCATGTTGCCTGGCCTGTAGAT | | |
| | TGGCCTTATCAGGTTTCTGGCTGCCTCTGCCTTAAGATCCTGAAGGCAAATTTTGTTTCA | | |
| | ACAGTTTGGAAGTCATCTGTGCGTCCAGCTTGACTTTGGACGAATAAGAAGATACTTCTA | | |
| | GAGTATGGCAATGATTCCAGATAATTTCTGGGATTTGAATCTACTTGAGTTTAAGGGCCT | | |
| | GGGACCTAATTTGGTTTAGTATAGAATTTGAAGAATTAATTTATAGGCAGCTGAATACCC | | |
| | AAAACTTGGGTGGTCGTCCTGTGGTTTGGCTGAGCTGTCCGGGCATAACCTGGTTCTCTC | | |
| | TTATGTTAAGGCTTTCTGGGAAGCCAGCCACTCTCCGCAGGAGTGAAACATGAAGTTGTT | | |
| | TTCTGAGGACCTGTTTTGGTCCGATTGTTTCCGCAGAGGACTGTGTTTATGCAGGGCAAA | | |
| | TCCCAGAAAGATAAGACGAAGCTAGAGAAACTTAATGTACCTGAATTCTTCATGGTGTAT | | |
| | TTGCAAACTAACTTAACATAGATTCTTTTGACTATGGTAAGTTTCAATCTCTCCTTGCCA | | |
| | AACAACATTATAAGTTTAGTTTTCTTCTTCCTCTTGCACCCGGTACGGAAAGGTGTAAGT | | |
| | GGTCGCTGAAAATTGAGGAACCTTCATCTGACCAATGTGGGTGCTCGTTTCTTGTGAAAT | | |
| | GTGTCCCTAAGCCTCCTTCTCCTTGCAGGCAGCCACCCACCCAGGTGTCTAAGATAGGAC | | |
| | ATGCTCCTTTCTTTCTCTAATCCCATCCTGAGCTTGCCGGCAAAGCCAATATGACCACTA | | |
| | CTGAGAAATAGTAATGACTTCTACAAATGCAAGGGTCTTACCCTCCTCTTTCCCTTAAAC | | |
| | ACCCTCCCTTTTCCTTACACCCCGTTTTTCCCATCCCCCAAATGTGTGGTATGGTGAAAC | | |
| | TAATCCCCTGAATGTGAATTGCTATCCTTATTGCCCTATTAAAGAAGAGCCAGCTGGTAT | | |
| | ATTGTCAGGAAGCACTATTTAAAATGTGAACTGTTATAGAGTAAATAAATAAATACTCTA | | |
| | CAGC | | |
| | ORF Start: ATG at 61 | | ORF Stop: TAA at 1927 |
| | SEQ ID NO: 80 | 622 aa | MW at 67037.7kD |

| | |
|---|---|
| NOV24a, | MATVLSRALKLPGKKSPDLGEYDPLTQADSDESEDDLVLNLQKNGGVKNGKSPLGEAPEP |
| CG141580-O1 | |
| Protein Sequence | DSDAEVAEAAKPHLSEVTTEGYPSEPLCGLEQKAASSLVSYVRTSVFLLTLGISMILVLL |
| | CAFLIPCPPRDLHSTWSRHLGSQGGGDLSPLELADVNGDGLRDVLLSFVMSRNGSAVGVS |
| | RPAANLVCLSGMNGSTLWSSLLPEEARDITCLELMPGSLAETICLVTGTHKMLSAFNATS |
| | GKAIWTLNPNYLSNGTLAAPVVVLPDLDEDGVRDLVVLAIGELQPDLCFLLVSGRTGNPV |
| | GRPVKYNIVGVGNLICPQVYITTNGAVYILFGFGNIQAVALRDIFVQAQNRDSSPPSLQI |
| | EEPEWEKRRSINLSELIDVYSDGVELLQMVKAPDSNCSNLLITTRQSLVLLRGQNLTPYW |
| | ALRLQGLRSQPTPGYFTDDQTLDFLLQIQDGVGMKKMMVVDGDSGSIVWSYRAPCHMKET |
| | PATSAVTSDQKSVFLFWAEGLSAASPNSDIILGTEPPSLHHLYLLHPAFPSILLDLANTT |
| | GTVTASEVGINDLWKDAFYVTRTTGPSSEGHPAALVVSKLSLRWALMEGQMAQLQESTPK |
| | IGRGELRRFLSRIKFVEAPYEI |

Two polymorphic variants of NOV24a have been identified and are shown in Table 411. Further analysis of the NOV24a protein yielded the following properties shown in Table 24B.

TABLE 24B
Protein Sequence Properties NOV24a

| | |
|---|---|
| PSort | 0.6000 probability located in plasma membrane; |
| analysis: | 0.4000 probability located in Golgi body; |
| | 0.3000 probability located in endoplasmic |
| | reticulum (membrane); 0.3000 probability |
| | located in microbody (peroxisome) |
| SignalP | No Known Signal Sequence Predicted |
| analysis: | |

A search of the NOV24a protein against the Geneseq database, a proprietary database that contains sequences published in patents and patent publication, yielded several homologous proteins shown in Table 24C.

TABLE 24C
Geneseq Results for NOV24a

| | | NOV24a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| ABB04610 | Human quinoprotein | 1 . . . 284 | 283/284 (99%) | e−160 |
| | dehydrogenase 33 protein | 1 . . . 284 | 283/284 (99%) | |
| | SEQ ID NO: 2 - Homo | | | |
| | sapiens, 302 aa. | | | |
| | [CN1307126-A, 08 AUG. | | | |
| | 2001] | | | |
| ABB05665 | Human transmembrane | 61 . . . 615 | 146/565 (25%) | 3e−46 |
| | protein clone amy2_{—} | 6 . . . 548 | 261/565 (45%) | |
| | 11d2 #2 - Homo sapiens, | | | |
| | 552 aa. [WO200198454- | | | |
| | A2, 27 DEC. 2001] | | | |
| ABB89951 | Human polypeptide SEQ ID | 61 . . . 615 | 145/565 (25%) | 1e−45 |
| | NO 2327 - Homo sapiens, | 6 . . . 548 | 260/565 (45%) | |
| | 552 aa. [WO200190304-A2, | | | |
| | 29 NOV. 2001] | | | |
| ABB89787 | Human polypeptide SEQ ID | 232 . . . 324 | 83/99 (83%) | 3e−39 |
| | NO 2163 - Homo sapiens, | 1 . . . 99 | 87/99 (87%) | |
| | 121 aa. [WO200190304-A2, | | | |
| | 29 NOV. 2001] | | | |
| ABB62154 | Drosophila melanogaster | 125 . . . 465 | 86/378 (22%) | 5e−14 |
| | polypeptide SEQ ID NO | 153 . . . 502 | 145/378 (37%) | |
| | 13254 - Drosophila | | | |
| | melanogaster, 989 aa. | | | |
| | [WO200171042-A2, 27 SEP. | | | |
| | 2001] | | | |

In a BLAST search of public sequence datbases, the NOV24a protein was found to have homology to the proteins shown in the BLASTP data in Table 24D.

TABLE 24D
Public BLASTP Results for NOV24a

| | | NOV24a | Identities/ | |
|---|---|---|---|---|
| Protein | | Residues/ | Similarities | |
| Accession | | Match | for the Matched | Expect |
| Number | Protein/Organism/Length | Residues | Portion | Value |
| Q9CXB0 | 8430419L09Rik protein - | 1 . . . 622 | 544/624 (87%) | 0.0 |
| | Mus musculus (Mouse), 624 | 1 . . . 624 | 580/624 (92%) | |
| | aa. | | | |
| Q9P261 | KIAA1467 protein - Homo | 191 . . . 622 | 432/432 (100%) | 0.0 |
| | sapiens (Human), 432 aa | 1 . . . 432 | 432/432 (100%) | |
| | (fragment). | | | |
| Q99L10 | Similar to RIKEN cDNA | 440 . . . 622 | 152/183 (83%) | 5e−84 |
| | 8430419L09 gene - Mus | 1 . . . 183 | 164/183 (89%) | |
| | musculus (Mouse), 183 aa | | | |
| | (fragment). | | | |
| Q96S30 | Hypothetical 69.3 kDa | 61 . . . 615 | 145/558 (25%) | 1e−46 |
| | protein - Homo sapiens | 72 . . . 605 | 261/558 (45%) | |
| | (Human), 636 aa. | | | |
| Q9HOX4 | Hypothetical 59.7 kDa | 61 . . . 615 | 146/565 (25%) | 8e−46 |
| | protein - Homo sapiens | 6 . . . 548 | 261/565 (45%) | |
| | (Human), 552 aa. | | | |

PFam analysis predicts that the NOV24a protein contains the domains shown in Table 24E.

TABLE 24E
Domain Analysis of NOV24a

| | | | |
|---|---|---|---|
| Pfam | NOV24a Match | Identities/ | Expect |
| Domain | Region | Similarities | Value |
| | | for the Matched | |
| | | Region | |

### Example 25

The NOV25 clone was analyzed, and the nucleotide and encoded polypeptide sequences are shown in Table 25A.

TABLE 25A
NOV25 Sequence Analysis

| | SEQ ID NO: 81 | 905 bp | |
|---|---|---|---|
| NOV25a, | AACAGCGGCCCTGCGGCTGGCGCGGCGGACGGGATGAGGCGCTGCAGTCTCTGCGCTTTC | | |
| CG141643-01 | | | |
| DNA Sequence | GACGCCGCCCGGGGGCCCAGGCGGCTGATGCGTGTGGGCCTCGCGCTCATCTTGGTGGGC | | |
| | CACGTGAACCTGCTGCTGGGGGCCGTGCTGCATGGCACCCTCCTGCGCCACCTCGCCAAT | | |
| | CCCCGCCGCGCTCTCACCCCGGAGTACACCGTAGCCAATGTCATCTCTGTCGGCTCGGGG | | |
| | CTGCTGGTGAGCGCGGCAGGCGACCCGGGCGGGGGCCGGGCTCCCGGAGAGCCCAGCAGG | | |
| | CCAAAGGCTTTGTGTCTTCCACAGAGCGTTTCCGTGGGACTTGTGGCCCTCCTGGCGTCC | | |
| | AGGAACCTTCTTCGCCCTCCACTGCACTGCCTCCTGCTGCCACTAGCTCTGGTGAACCTG | | |
| | CTCTTGTCCGTTGCCTGCTCCCTGGGCCTCCTTCTTCCTGTGTCACTCACTGTGGCcAAC | | |
| | GGTGGCCGCCGCCTTATTGCTGACTGCCACCCAGGACTGCTGGATCCTCTCGTACCACTG | | |
| | GATGAGGGGCCGGGACATACTGACTGCCCCTTTGACCCCACAAGAATCTATGATACAGCC | | |
| | TTGGCTCTCTCGATCCCTTCTTTGCTCATGTCTGCAGGGGAGGCTGCTCTATCTCGTTAC | | |
| | TGCTGTGTGGCTGCACTCACTCTACGTGGAGTTGGGCCCTGCAGGAAGGACGGACTTCAG | | |
| | GGGCAGGTAGTAGCTGGGTGTGACGCAAGAGTGAAACAGAAAGCCTGGCAGCCACGGTTT | | |
| | CCTGGCATTAAACTCAAAGCATTATGAATATGCCACTAAAGTGACTCAGCTACCAGACCA | | |
| | ATGATCCTGTAAGGCAGCCACAGAACTAAAAAACAACAATTATTATTAAACTGCTCTGGA | | |
| | TTCTC | | |
| | ORF Start: ATG at 34 | | ORF Stop: TGA at 805 |
| | SEQ ID NO: 82 | 257 aa | MW at 26717.2kD |

| | |
|---|---|
| NOV25a, | MRRCSLCAFDAARGPRRLMRVCLALILVGHVNLLLGAVLHGTVLRIVANPRGAVTPEYTV |
| CG141643-01 | |
| Protein Sequence | ANVISVGSGLLVSAAGDPGGGRAPGEPSRPKALCLPQSVSVGLVALLASRNLLRPPLHWV |
| | LLALALVNLLLSVACSLGLLLAVSLTVANGGRRLIADCHPGLLDPLVPLDEGPGHTDCPF |
| | DPTRIYDTALALWIPSLLMSAGEAALSGYCCVAALTLRGVGPCRKDGLQGQVVACCDARV |
| | KQKAWQPRFPGIKVKAL |

Further analysis of the NOV25a protein yielded the following properties shown in Table 25B.

TABLE 25B
Protein Sequence Properties NOV25a

| | |
|---|---|
| PSort | 0.6400 probability located in plasma membrane; |
| analysis: | 0.4600 probability located in Golgi body; |
| | 0.3700 probability located in endoplasmic |
| | reticulum (membrane); 0.1000 probability |
| | located in endoplasmic reticulum (lumen) |
| SignalP | Cleavage site between residues 37 and 38 |
| analysis: | |

A search of the NOV25a protein against the Geneseq database, a proprietary database that contains sequences published in patents and patent publication, yielded several homologous proteins shown in Table 25C.

TABLE 25C
Geneseq Results for NOV25a

| | | NOV25a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAY78805 | Hydrophobic domain | 1 . . . 257 | 231/257 (89%) | e−127 |
| | containing protein clone | 1 . . . 231 | 231/257 (89%) | |
| | HP10508 protein sequence - | | | |
| | Homo sapiens, 231 aa. | | | |
| | [WO200000506-A2, 06 | | | |
| | JAN. 2000] | | | |
| ABB90256 | Human polypeptide SEQ ID | 1 . . . 232 | 205/232 (88%) | e−111 |
| | NO 2632 - Homo sapiens, | 1 . . . 206 | 206/232 (88%) | |
| | 240 aa. [WO200190304-A2, | | | |
| | 29 NOV. 2001] | | | |
| AAU83615 | Human PRO protein, Seq ID | 19 . . . 232 | 187/214 (87%) | 1e−99 |
| | No 48 - Homo sapiens, 222 | 1 . . . 188 | 188/214 (87%) | |
| | aa. [WO200208288-A2, 31 | | | |
| | JAN. 2002] | | | |
| AAG81326 | Human AFP protein | 19 . . . 232 | 187/214 (87%) | 1e−99 |
| | sequence SEQ ID NO: 170 - | 1 . . . 188 | 188/214 (87%) | |
| | Homo sapiens, 222 aa. | | | |
| | [WO200129221-A2, 26 | | | |
| | APR. 2001] | | | |
| AAB43588 | Human cancer associated | 102 . . . 232 | 127/131 (96%) | 9e−70 |
| | protein sequence SEQ ID | 79 . . . 209 | 129/131 (97%) | |
| | NO: 1033 - Homo sapiens, | | | |
| | 243 aa. [WO200055350-A1, | | | |
| | 21 SEP. 2000] | | | |

In a BLAST search of public sequence datbases, the NOV25a protein was found to have homology to the proteins shown in the BLASTP data in Table 25D.

TABLE 25D
Public BLASTP Results for NOV25a

| | | NOV25a | Identities/ | |
|---|---|---|---|---|
| Protein | | Residues/ | Similarities for | |
| Accession | | Match | the Matched | Expect |
| Number | Protein/Organism/Length | Residues | Portion | Value |
| AAH27812 | Similar to RIKEN cDNA | 1 . . . 232 | 185/232 (79%) | e−100 |
| | 2010001C09 gene - Mus | 10 . . . 215 | 194/232 (82%) | |
| | musculus (Mouse), 249 aa. | | | |
| CAC38576 | Sequence 169 from Patent | 19 . . . 232 | 187/214 (87%) | 4e−99 |
| | WO0129221 - Homo sapiens | 1 . . . 188 | 188/214 (87%) | |
| | (Human), 222 aa. | | | |
| Q9D817 | 2010001C09Rik protein - | 1 . . . 232 | 163/232 (70%) | 3e−82 |
| | Mus musculus (Mouse), 223 | 10 . . . 189 | 171/232 (73%) | |
| | aa. | | | |
| Q969K7 | Hypothetical 23.8 kDa | 18 . . . 210 | 66/193 (34%) | 6e−26 |
| | protein (Similar to RIKEN | 17 . . . 177 | 104/193 (53%) | |
| | cDNA 1810017F10 gene) | | | |
| | (Beta-casein-like protein) - | | | |
| | Homo sapiens (Human), 222 | | | |
| | aa. | | | |
| Q8VCL0 | RIKEN cDNA 1810017F10 | 18 . . . 210 | 69/195 (35%) | 1e−24 |
| | gene - Mus musculus | 17 . . . 177 | 101/195 (51%) | |
| | (Mouse), 219 aa. | | | |

PFam analysis predicts that the NOV25a protein contains the domains shown in Table 25E.

TABLE 25E
Domain Analysis of NOV25a

| | | | |
|---|---|---|---|
| | | Identities/ | |
| | | Similarities | |
| | NOV25a | for the Matched | |
| Pfam Domain | Match Region | Region | Expect Value |

### Example 26

The NOV26 clone was analyzed, and the nucleotide and encoded polypeptide sequences are shown in Table 26A.

TABLE 26A
NOV26 Sequence Analysis

| | SEQ ID NO: 83 | 446 bp | |
|---|---|---|---|
| NOV26a, | CTGGGGATAGAGCCTCCTCAAATCCAAATGCTACCAGCTCCAGCTCCCAGGATCCAGACA | | |
| CG142003-01 | | | |
| DNA Sequence | GTTTGCAAGACAGAGGCGAAGGGAAGGTCGCAACAACAGTTATCTCCAAGATGCTATTCG | | |
| | TTGAACCCATCCTGGAGGTTTCCAGCTTGCCGACAACCAACTCAACAACCAATTCAGCCA | | |
| | CCAAAATAACAGCTAATACCACTGATGAACCCACCACACAACCCACCACAGAGGACCCAG | | |
| | ATCTTCACGTTTCTGCGATGCACCACCAGACAGTGCTGGAACTGACAGAGACTGGGGTGG | | |
| | AGGTGGCTCCAGCCTCCGCCATCTCTGTGGCCCGCACCCTGCTGGTCTTTGAACTCCAGC | | |
| | AGCCCTTCCTCTTCCTGCTCTCGGACCAGCACCACAAGTTCCCTGTCTTCATGGGGCGAG | | |
| | TATATGACCCCAGCGCCTGAGACAAG | | |
| | ORF Start: at 3 | | ORF Stop: TGA at 438 |
| | SEQ ID NO: 84 | 145 aa | MW at 15697.3kD |
| NOV26a, | GDRASSNPNATSSSSQDPESLQDRGECKVATTVTSKMLFVEPILEVSSLPTTNSTTNSAT | | |
| CG142003-01 | | | |
| Protein Sequence | KITANTTDEPTTQPTTEDPDLQVSAMQHQTVLELTETGVEVAAASAISVARTLLVFEVQQ | | |
| | PFLFVLWDQQHKFPVFMCRVYDPRA | | |
| | SEQ ID NO: 85 | 436 bp | |
| NOV26b, | CACCAAGCTTAATCCAAATGCTACCAGCTCCAGCTCCCAGGATCCAGAGAGTTTGCAAGA | | |
| 306076006 DNA | | | |
| Sequence | CAGAGGCGAAGGGAAGGTCGCAACAACAGTTATCTCCAAGATGCTATTCGTTGAACCCAT | | |
| | CCTGGAGGTTTCCAGCTTGCCGACAACCAACTCAACAACCAATTCAGCCACCAAAATAAC | | |
| | AGCTAATACCACTGATGAACCCACCACACAACCCACCACAGAGGACCCAGATCTTCAGGT | | |
| | TTCTGCGATGCAGCACCAGACAGTGCTGGAACTGACAGAGACTGGGGTGGAGGTGGCTGC | | |
| | AGCCTCCGCCATCTCTCTGGCCCGCACCCTGCTGGTCTTTGAAGTGCAGCAGCCCTTCCT | | |
| | CTTCGTGCTCTGGGACCAGCAGCACAAGTTCCCTCTCTTCATGGGGCGAGTATATGACCC | | |
| | CAGCGCCCTCGAGGGC | | |
| | ORF Start: at 2 | | ORF Stop: end of sequence |
| | SEQ ID NO: 86 | 145 aa | MW at 15765.5kD |
| NOV26b, | TKLNPNATSSSSQDPESLQDRGEGKVATTVISKMLFVEPILEVSSLPTTNSTTNSATKIT | | |
| 306076006 | | | |
| Protein Sequence | ANTTDEPTTQPTTEDPDLQVSADQHQTVLELTETGVEVAAASAISVARTLLVPEVQQPFL | | |
| | FVLWDQQHKFPVFMGRVYDPRALEG | | |
| | SEQ ID NO: 87 | 223 bp | |
| NOV26c, | CACCAAGCTTACAGAGGACCCAGATCTTCAGGTTTCTGCGATGCAGCACCAGACAGTGCT | | |
| 278889088 DNA | | | |
| Sequence | GGAACTGACAGAGACTGCCGTGGAGGTGGCTGCAGCCTCCGCCATCTCTGTGGCCCGCAC | | |
| | CCTGCTGGTCTTTGAAGTGCAGCAGCCCTTCCTCTTCGTGCTCTGGGACCAGCAGCACAA | | |
| | GTTCCCTGTCTTCATGGGGCGAGTATATGACCCCCTCGAGGGC | | |
| | ORF Start: at 2 | | ORF Stop: end of sequence |
| | SEQ ID NO: 88 | 74 aa | MW at 8317.5kD |
| NOV26c, | TKLTEDPDLQVSAMQHQTVLELTETGVEVAAASAISVARTLLVFEVQQPFLFVLWDQQHK | | |
| 278889088 | | | |
| Protein Sequence | FPVFMGRVYDPLEG | | |
| | SEQ ID NO: 89 | 529 bp | |
| NOV26d, | GAGGAGAAGTTTGGACTCCGCTGACGTCGCCGCCCAGATGGCCTCCAGGCTGACCCTGCT | | |
| CG142003-02 | | | |
| DNA Sequence | GACCCTCCTGCTGCTGCTGCTGGCTGGGGATAGAGCCTCCTCAAATCCAAATGCTACCAG | | |
| | CTCCAGCTCCCAGGATCCACAGAGTTTGCAAGACAGAGGCCAAGGGAAGGTCCCAACAAC | | |
| | AGTTATCTCCAACATGCTATTCGTTGAACCCATCCTGCAGGTTTCCAGCTTGCCGACAAC | | |
| | CAACTCAACAACCAATTCAGCCACCAAAATAACACCTAATACCACTGATGAACCCACCAC | | |
| | ACAACCCACCACACAGGACCCAGATCTTCAGGTTTCTGCCATGCAGCACCAGACAGTCCT | | |
| | GCAACTGACAGAGACTCCGGTCGAGGTGCCTGCACCCTCCGCCATCTCTGTGGCCCGCAC | | |
| | CCTGCTGGTCTTTGAAGTGCACCAGCCCTTCCTCTTCGTCCTCTGGGACCAGCACCACAA | | |
| | GTTCCCTGTCTTCATGGGGCGAGTATATGACCCCAGGGCCTGAGACAAG | | |
| | ORF Start: ATG at 38 | | ORF Stop: TGA at 521 |
| | SEQ ID NO: 90 | 161 aa | MW at 17434.5kD |

| | |
|---|---|
| NOV26d, | MASRLTLLTLLLLLLAGERASSNPNATSSSSQDPESLQDRGEGKVATTVISKMLFVEPTL |
| CG142003-02 | |
| Protein Sequence | EVSSLPTTNSTTNSATKITANTTDEPTTQPTTEDPDLQVSAMQHQTVLELTETGVEVAAA |
| | SAISVARTLLVFEVQQPFLFVLWDQQHKFPVFMGRVYDPRA |

Sequence comparison of the above protein sequences yields the following sequence relationships shown in Table 26B.

TABLE 26B
Comparison of NOV26a against NOV26b through NOV26d.

| | | Identities/ |
|---|---|---|
| | NOV26a Residues/ | Similarities for |
| Protein Sequence | Match Residues | the Matched Region |
| NOV26b | 7 . . . 145 | 99/139 (71%) |
| | 4 . . . 142 | 99/139 (71%) |
| NOV26c | 76 . . . 143 | 58/68 (85%) |
| | 4 . . . 71 | 58/68 (85%) |
| NOV26d | 1 . . . 145 | 93/145 (64%) |
| | 17 . . . 161 | 93/145 (64%) |

One polymorphic variant of NOV26a has been identified and is shown in Table 41J. Further analysis of the NOV26a protein yielded the following properties shown in Table 26C.

TABLE 26C
Protein Sequence Properties NOV26a

| | |
|---|---|
| PSort analysis: | 0.6500 probability located in cytoplasm; |
| | 0.1555 probability located in lysosome (lumen); |
| | 0.1000 probability located in mitochondrial matrix |
| | space; 0.0000 probability located in endoplasmic |
| | reticulum (membrane) |
| SignalP analysis: | No Known Signal Sequence Predicted |

A search of the NOV26a protein against the Geneseq database, a proprietary database that contains sequences published in patents and patent publication, yielded several homologous proteins shown in Table 26D.

TABLE 26D
Geneseq Results for NOV26a

| | | NOV26a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU02972 | Angiotensin converting | 1 . . . 94 | 81/94 (86%) | 3e−37 |
| | enzyme (ACEV) splice | 17 . . . 109 | 83/94 (88%) | |
| | variant protein #72 - Homo | | | |
| | sapiens, 636 aa. | | | |
| | [WO200136632-A2, 25- | | | |
| | MAY-2001] | | | |
| AAW18207 | Wild-type C1 inhibitor - | 1 . . . 94 | 81/94 (86%) | 3e−37 |
| | Homo sapiens, 500 aa. | 17 . . . 109 | 83/94 (88%) | |
| | [US5622930-A, 22-APR- | | | |
| | 1997] | | | |
| AAW18212 | Recombinant C1 inhibitor | 1 . . . 94 | 81/94 (86%) | 3e−37 |
| | mutein - Homo sapiens, 500 | 17 . . . 109 | 83/94 (88%) | |
| | aa. [US5622930-A, 22-APR- | | | |
| | 1997] | | | |
| AAW18218 | Recombinant C1 inhibitor | 1 . . . 94 | 81/94 (86%) | 3e−37 |
| | mutein - Homo sapiens, 500 | 17 . . . 109 | 83/94 (88%) | |
| | aa. [US5622930-A, 22-APR- | | | |
| | 1997] | | | |
| AAW18217 | Recombinant C1 inhibitor | 1 . . . 94 | 81/94 (86%) | 3e−37 |
| | mutein - Homo sapiens, 500 | 17 . . . 109 | 83/94 (88%) | |
| | aa. [US5622930-A, 22-APR- | | | |
| | 1997] | | | |

In a BLAST search of public sequence datbases, the NOV26a protein was found to have homology to the proteins shown in the BLASTP data in Table 26E.

TABLE 26E
Public BLASTP Results for NOV26a

| | | NOV26a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96FE0 | Serine (or cysteine) proteinase | 1 . . . 94 | 81/94 (86%) | 8e−37 |
| | inhibitor, clade G (C1 | 17 . . . 109 | 83/94 (88%) | |
| | inhibitor), member 1 - Homo | | | |
| | sapiens (Human), 500 aa. | | | |
| P05155 | Plasma protease C1 inhibitor | 1 . . . 94 | 81/94 (86%) | 8e−37 |
| | precursor (C1 Inh) (C1 Inh) - | 17 . . . 109 | 83/94 (88%) | |
| | Homo sapiens (Human), 500 | | | |
| | aa. | | | |
| Q95J12 | Complement C1 inhibitor - Pan- | 2 . . . 82 | 75/81 (92%) | 3e−34 |
| | troglodytes (Chimpanzee), 162 | 1 . . . 80 | 77/81 (94%) | |
| | aa (fragment). | | | |
| Q16304 | C1-inhibitor - Homo sapiens | 76 . . . 145 | 67/70 (95%) | 7e−32 |
| | (Human), 83 aa (fragment). | 14 . . . 83 | 68/70 (96%) | |
| P97290 | Plasma protease C1 inhibitor | 76 . . . 144 | 57/69 (82%) | 2e−27 |
| | precursor (C1 Inh) (C1 Inh) - | 435 . . . 503 | 65/69 (93%) | |
| | Mus musculus (Mouse), 504 | | | |
| | aa. | | | |

PFam analysis predicts that the NOV26a protein contains the domains shown in Table 26F.

TABLE 26F
Domain Analysis of NOV26a

| | | Identities/ | |
|---|---|---|---|
| | | Similarities | |
| | NOV26a | for the Matched | |
| Pfam Domain | Match Region | Region | Expect Value |
| serpin | 76 . . . 143 | 31/74 (42%) | 2.5e−25 |
| | | 61/74 (82%) | |

### Example 27

The NOV27 clone was analyzed, and the nucleotide and encoded polypeptide sequences are shown in Table 27A.

TABLE 7A
NOV27 Sequence Analysis

| | SEQ ID NO: 91 | 1356 bp | |
|---|---|---|---|
| NOV27a, | GGCGAGGCCGGCGCATGCGGCAGCTGTCCCGGGGCCGCGTGCTGGGCATCTCGGTGGCC | | |
| CG142023-01 | | | |
| DNA Sequence | ATCGCGCACGGGGTCTTCTCGGGCTCCCTCAACATCTTGCTCAAGTTCCTCATCAGCCGC | | |
| | TACCAGTTCTCCTTCCTGACCCTGGTGCAGTGCCTGACCAGCTCCACCGCGGCGCTGAGC | | |
| | CTGGAGCTGCTCCGGCGCCTCGGGCTCATCGCCGTGCCCCCCTTCGGTCTGAGCCTCGCG | | |
| | CGCTCCTTCGCGGGGGTCGCGGTGCTCTCCACGCTGCAGTCCAGCCTCACGCTCTGGTCC | | |
| | CTGCGCGGCCTCAGCCTGCCCATCTACGTGGTCTTCAAGCGCTGCCTGCCCCTGGTCACC | | |
| | ATGCTCATCGGCGTCCTGGTGCTCAAGAACGGCGCGCCCTCGCCAGGGGTGCTGGCGGCG | | |
| | GTGCTCATCACCACCTGCGGCGCCGCCCTGGCAGGTCCCGGCGACCTGACGGGCGACCCC | | |
| | ATCGGGTACGTCACGGGAGTGCTGGCGGTGCTGGTCCACGCTGCCTACCTGGTGCTCATC | | |
| | CAGAAGGCCAGCGCAGACACCGAGCACGGGCCGCTCACCGCGCAGTACGTCATCGCCGTC | | |
| | TCTGCCACCCCGCTGCTGGTCATCTGCTCCTTCGCCAGCACCGACTCCATCCACGCCTGG | | |
| | ACCTTCCCGGGCTGGAAGGACCCGGCCATGGTCTGCATCTTCGTGGCCTGCATCCTGATC | | |
| | GGCTGCGCCATGAACTTCACCACGCTGCACTGCACCTACATCAATTCGGCCGTGACCACC | | |
| | AGCTTCGTGGGTGTGGTGAAGAGCATCGCCACCATCACGGTGGGCATGGTGGCCTTCAGC | | |
| | GACGTGGACCCCACCTCTCTGTTCATTGCCGGCGTGGTGGTGAACACCCTGGGCTCTATC | | |
| | ATTTACTGTGTGGCCAAGTTCATGGAGACCAGAAAGCAAAGCAACTACGAGGACCTGGAG | | |
| | GCCCACCCTCGGGGAGAGGAGGCGCAGCTAAGTGGACACCAGCTGCCGTTCGTGATGGAG | | |
| | GAGCTGCCCGGGGACGGAGGAAATGGCCGGTCACAAGGTGGGGAGGCAGCAGGTGGCCCC | | |
| | GCTCAGGAGAGCAGCCAAGAGGTCAGGGGCAGCCCCCGACGAGTCCCGCTGGTGGCTGGG | | |
| | AGCTCTGAAGAAGGGAGCAGGAGGTCGTTAAAAGATGCTTACCTCGAGGTATGGAGGTTG | | |
| | GTTAGGGGAACCAGGTATATGAAGAAGGATTATTTGATAGAAAACGAGGAGTTACCCAGT | | |
| | CCTTGAGAAGGAGGTGCATGTACGTACCTATGTGCATACACTTATTTTATATGTTAGAAA | | |
| | TGACGTGTTTTAATGAGAGGCCTCCCCGTTTTATTC | | |
| | ORF Start: ATG at 16 | | ORF Stop: TGA at 1264 |
| | SEQ ID NO: 92 | 416 aa | MW at 44181.9kD |

| | |
|---|---|
| NOV27a, | MRQLCRGRVLGISVAIAHGVFSGSLNTLLKFLTSRYQFSFLTLVQCLTSSTAALSLELLR |
| CG142023-01 | |
| Protein Sequence | RLGLIAVPPFCLSLARSFAGVAVLSTLQSSLTLWSLRGLSLPMYVVFKRCLPLVTMLIGV |
| | LVLKNGAPSPGVLAAVLITTCGAALAGAGDLTGDPIGYVTGVLAVLVHAAYLVLIQKASA |
| | DTEHGPLTAQYVIAVSATPLLVICSFASTDSTHAWTFPGWKDPAMVCIFVACTLIGCAMN |
| | FTTLHCTYINSAVTTSFVGVVKSIATITVGMVAFSDVEPTSLFIAGVVVNTLGSIIYCVA |
| | KFMETRKQSNYEDLEAQPRGEEAQLSGDQLPFVMEELPGEGGNGRSEGGEAAGGPAQESR |
| | QEVRGSPRGVPLVAGSSEEGSRRSLKDAYLEVWRLVRGTRYMKKDYLIENEELPSP |

Further analysis of the NOV27a protein yielded the following properties shown in Table 27B.

TABLE 27B
Protein Sequence Properties NOV27a

| | |
|---|---|
| PSort analysis: | 0.6400 probability located in plasma membrane; |
| | 0.4600 probability located in Golgi body; |
| | 0.3700 probability located in endoplasmic reticulum |
| | (membrane); 0.1000 probability located in |
| | endoplasmic reticulum (lumen) |
| SignalP analysis: | Cleavage site between residues 20 and 21 |

A search of the NOV27a protein against the Geneseq database, a proprietary database that contains sequences published in patents and patent publication, yielded several homologous proteins shown in Table 27C.

TABLE 27C
Geneseq Results for NOV27a

| | | NOV27a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU81226 | Human lung cancer protein, | 1 . . . 416 | 391/416 (93%) | 0.0 |
| | Seq ID No 62 - Homo | 1 . . . 391 | 391/416 (93%) | |
| | sapiens, 391 aa. | | | |
| | [WO200192525-A2, 06- | | | |
| | DEC-2001] | | | |
| AAM47572 | Drosophila cell cycle | 12 . . . 321 | 87/316 (27%) | 3e−21 |
| | progression protein #1 - | 64 . . . 371 | 153/316 (47%) | |
| | Drosophila sp, 373 aa. | | | |
| | [WO200172774-A2, 04- | | | |
| | OCT-2001] | | | |
| ABB60236 | Drosophila melanogaster | 12 . . . 321 | 87/316 (27%) | 3e−21 |
| | polypeptide SEQ ID NO | 64 . . . 371 | 153/316 (47%) | |
| | 7500 - Drosophila | | | |
| | melanogaster, 373 aa. | | | |
| | [WO200171042-A2, 27-SEP- | | | |
| | 2001] | | | |
| AAB88597 | Human hydrophobic domain | 8 . . . 322 | 74/315 (23%) | 7e−14 |
| | containing protein clone | 24 . . . 329 | 137/315 (43%) | |
| | HP03670 #121 - Homo | | | |
| | sapiens, 337 aa. | | | |
| | [WO200112660-A2, 22- | | | |
| | FEB-2001] | | | |
| AAB56473 | Human prostate cancer | 8 . . . 322 | 74/315 (23%) | 1e−13 |
| | antigen protein sequence | 28 . . . 333 | 136/315 (42%) | |
| | SEQ ID NO: 1051 - Homo | | | |
| | sapiens, 341 aa. | | | |
| | [WO200055174-A1, 21-SEP- | | | |
| | 2000] | | | |

In a BLAST search of public sequence datbases, the NOV27a protein was found to have homology to the proteins shown in the BLASTP data in Table 27D.

TABLE 27D
Public BLASTP Results for NOV27a

| | | NOV27a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9CXD4 | 6230421J19Rik protein - Mus | 271 . . . 416 | 111/152 (73%) | 4e−55 |
| | musculus (Mouse), 152 aa. | 1 . . . 152 | 120/152 (78%) | |
| Q94B65 | Hypothetical 34.6 kDa protein - | 10 . . . 319 | 93/316 (29%) | 8e−34 |
| | Arabidopsis thaliana | 13 . . . 323 | 163/316 (51%) | |
| | (Mouse-ear cress), 323 aa. | | | |
| Q9SB76 | Hypothetical 31.9 kDa protein - | 30 . . . 319 | 90/296 (30%) | 1e−31 |
| | Arabidopsis thaliana | 6 . . . 296 | 151/296 (50%) | |
| | (Mouse-ear cress), 296 aa. | | | |
| Q95YI5 | UDP-sugar transporter | 12 . . . 321 | 87/316 (27%) | 9e−21 |
| | UST74c (Fringe connection | 64 . . . 371 | 153/316 (47%) | |
| | protein) - Drosophila | | | |
| | melanogaster (Fruit fly), 373 | | | |
| | aa. | | | |
| Q9NTN3 | UDP-glucuronic acid/UDP-N- | 18 . . . 309 | 80/295 (27%) | 1e−16 |
| | acetylgalactosamine | 49 . . . 341 | 132/295 (44%) | |
| | transporter (UDP- | | | |
| | GlcA/UDP-GalNAc | | | |
| | transporter) - Homo sapiens | | | |
| | (Human), 355 aa. | | | |

PFam analysis predicts that the NOV27a protein contains the domains shown in Table 27E.

TABLE 27E
Domain Analysis of NOV27a

| | | Identities/ | |
|---|---|---|---|
| | NOV27a | Similarities for the | |
| Pfam Domain | Match Region | Matched Region | Expect Value |
| DUF6 | 166 . . . 299 | 21/135 (16%) | 0.29 |
| | | 87/135 (64%) | |

### Example 28

The NOV28 clone was analyzed, and the nucleotide and encoded polypeptide sequences are shown in Table 28A.

TABLE 28A
NOV28 Sequence Analysis

| | SEQ ID NO: 93 | 785 bp | |
|---|---|---|---|
| NOV28a. | AAAAACTCCATCTGGGGCTCTTCATAGAAAAAGGAAAATGGCAGCCTGGCCCTTCTCCAG | | |
| CG142092-01 | | | |
| DNA Sequence | GCTCTGGAAAGTCTCTGATCCAATTCTCTTCCAAATGACCTTGATCCCTGCTCTCTTCCC | | |
| | TGCTGTTCTTGGCAATTGTGGTCCTCCACCCACTTTATCATTTGCTGCCCCGATCGATAT | | |
| | TACGTTGACTGAGACACGCTTCAAAACTGGAACTACTCTGAAATACACCTGCCTCCCTGG | | |
| | CTACGTCACATCCCATTCAACTCAGACGCTTACCTGTAATTCTGATGGCGAATGGGTGTA | | |
| | TAACACCTTCTGTATCTACAAACGATGCAGACACCCAGGAGAGTTACGTAATGGGCAAGT | | |
| | AGAGATTAAGACAGATTTATCTTTTGGATCACAAATAGAATTCAGCTGTTCAGAAGGATT | | |
| | TTTCTTAATTGGCTCAACCACTAGTCGTTGTGAAGTCCAAGATAGAGCAGTTGGCTGGGG | | |
| | TCATCCTCTCCCACAATGTGAAATTCTCAAGTGTAAGCCTCCTCCAGACATCAGGAATGG | | |
| | AAGGCACAGCGGTGAAGAAAATTTCTACGCATACGGCTTTTCTGTCACCTACAGCTGTGA | | |
| | ACAAGTGCTCACAGGCAAAAGACTCATGCAGTGTCTCCCAAACCCAGAGGATCTCAAAAT | | |
| | GGCCCTGGAGGTATATAAGCTGTCTCTGGAAATTGAACAACTGGAACTACAGAGAGACAG | | |
| | CGCAAGACAATCCACTTTGGATAAAGAACTATAATTTTTCTCAAAACAAGGAGGAAAAGG | | |
| | TGTCT | | |
| | ORF Start: at 2 | | ORF Stop: TAA at 752 |
| | SEQ ID NO: 94 | 250 aa | MW at 28139.0kD |
| NOV28a, | KTPSGALHRKRKMAAWPFSRLWKVSDPILFQMTLIAALLPAVLGNCGPPPTLSFAAPMDI | | |
| CG142092-01 | | | |
| Protein Sequence | TLTETRFKTGTTLKYTCLPGYVRSHSTQTLTCNSDGEWVYNTFCIYKRCRHPGELRNGQV | | |
| | EIKTDLSFGSQIEFSCSEGFFLIGSTTSRCEVQDRGVGWGHPLPQCEIVKCKPPPDIRNG | | |
| | RHSGEENFYAYGFSVTYSCEQVLTGKRLMQCLPNPEDVKMALEVYKLSLEIEQLELQRDS | | |
| | ARQSTLDKEL | | |
| | SEQ ID NO: 95 | 972 bp | |
| NOV28b, | AAAACTCTGATCTGGGGAGGAACCAGGACTACATAGATCAAGGCAGTTTTCTTCTTTGAG | | |
| CG142092-02 | | | |
| DNA Sequence | AAACTATCCCAGATATCATCATAGAGTCTTCTGCTCTTCCTCAACTACCAAAGAAAAACA | | |
| | TCAGCGAAGCAGCAGGCCATGCACCCCCCAAAAACTCCATCTGGGGCTCTTCATAGAAAA | | |
| | AGGAAAATGGCAGCCTGGCCCTTCTCCAGGCTGTGGAAAGTCTCTGATCCAATTCTCTTC | | |
| | CAAATCACCTTGATCGCTGCTCTGTTGCCTGCTGTTCTTGGCAATTGTGGTCCTCCACCC | | |
| | ACTTTATCATTTGCTGCCCCCATGGATATThCGTTCACTGAGACACGCTTCAAAACTCCA | | |
| | ACTACTCTCAAATACACCTGCCTCCCTGGCTACGTCACATCCCATTCAACTCAGACGCTT | | |
| | ACCTGTAATTCTCATGGCGAATGGGTCTATAACACCTTCTGTATCTACAAACGATCCAGA | | |
| | CACCCAGGAGAGTTACGTAATGGGCAAGTAGAGATTAAGACAGATTTATCTTTTGGATCA | | |
| | CAAATAGAATTCAGCTGTTCAGAAGGCTGTGAACAAGTGCTCACAGGCAAAAGACTCATG | | |
| | GAAATTGAACAACTGGAACTACAGAGAGACAGCGCAAGACAATCCACTTTCGATAAAGAA | | |
| | CTATAATTTTTCTCAAAACAAGGACGAAAACGTGTCTTGCTGGCTTGCCTCTTGCAATTC | | |
| | AATACAGATCAGTTTAGCAAATCTACTGTCAATTTCCCAGTGATATTCATCATAATAAAT | | |
| | ATCTAGAAATGATAATTTGCTAAAGTTTAGTGCTTTGAGATTGTGAAATTATTAATCATC | | |
| | CTCTGTGTGGCTCATGTTTTTGCTTTTCAACACACAAAGCACAAATTTTTTTTCGATTAA | | |
| | AAATGTATGTAT | | |
| | ORF Start: ATG at 139 | | ORF Stop: TAA at 724 |
| | SEQ ID NO: 96 | 195 aa | MW at 21984.2kD |
| NOV28b, | MHPPKTPSGALHRKRKMAAWPFSRLWKVSDPILFQMTLTAALLPAVLGNCCPPPTLSFAA | | |
| CG142092-02 | | | |
| Protein Sequence | PMDITLTETRFKTGTTLKYTCLPGYVRSHSTQTLTCNSDCEWVYNTFCIYKRCRHPGELR | | |
| | NGQVEIKTDLSFCSQIEFSCSEGCEQVLTCKRLMQCLPNPEDVKMALEVYKLSLEIEQLE | | |
| | LQRDSARQSTLDKEL | | |
| | SEQ ID NO: 97 | 681 bp | |
| NOV28c, | AAAACTCTGATCTGCGCACGAACCAGGACTACATAGATCAAGGCACTTTTCTTCTTTGAG | | |
| CG142092-03 | | | |
| DNA Sequence | AAACTATCCCAGATATCATCATAGACTCTTCTGCTCTTCCTCAACTACCAAAGAAAAACA | | |
| | TCAGCGAAGCAGCAGCCCATGCACCCCCCAAAAACTCCATCTGCGGCTCTTCATAGAAAA | | |
| | AGGAAAATGGCAGCCTGGCCCTTCTCCAGGCTGTGGAAAGTCTCTGATCCAATTCTCTTC | | |
| | CAAATGACCTTGATCGCTGCTCTGTTGCCTGCTGTTCTTGGCAATTGTGGTCCTCCACCC | | |
| | ACTTTATCATTTGCTGCCCCGATGGATATTACCTTGACTGAGACACGCTTCAAAACTGGA | | |
| | ACTACTCTGGAAATTGAACAACTGGAACTACACAGAGACAGCGCAAGACAATCCACTTTG | | |
| | GATAAAGAACTATAATTTTTCTCAAAAGAAGGAGGAAAAGGTGTCTTGCTGGCTTGCCTC | | |
| | TTGCAATTCAATACAGATCAGTTTAGCAAATCTACTGTCAATTTGGCAGTGATATTCATC | | |
| | ATAATAAATATCTACAAATGATAATTTCCTAAAGTTTAGTGCTTTGAGATTGTGAAATTA | | |
| | TTAATCATCCTCTGTGTGGCTCATGTTTTTGCTTTTCAACACACAAAGCACAAATTTTTT | | |
| | TTCGATTAAAAATGTATGTAT | | |
| | ORF Start: ATG at 139 | | ORF Stop: TAA at 433 |
| | SEQ ID NO: 98 | 98 aa | MW at 10927.6kD |

| | |
|---|---|
| NOV28c, | MHPPKTPSGALHRKRKMAAWPFSRLWKVSDPILFQMTLIAALLPAVLGNCCPPPTLSFAA |
| CG142092-03 | |
| Protein Sequence | PMDITLTETRFKTGTTLEIEQLELQRDSARQSTLDKEL |

Sequence comparison of the above protein sequences yields the following sequence relationships shown in Table 28B.

TABLE 28B
Comparison of NOV28a against NOV28b and NOV28c.

| | | Identities/ |
|---|---|---|
| | NOV28a Residues/ | Similarities for the |
| Protein Sequence | Match Residues | Matched Region |
| NOV28b | 1 . . . 250 | 185/250 (74%) |
| | 5 . . . 195 | 185/250 (74%) |
| NOV28c | 1 . . . 74 | 73/74 (98%) |
| | 5 . . . 78 | 74/74 (99%) |

Further analysis of the NOV28a protein yielded the following properties shown in Table 28C.

TABLE 28C
Protein Sequence Properties NOV28a

| | |
|---|---|
| PSort analysis: | 0.6500 probability located in plasma membrane; |
| | 0.5046 probability located in mitochondrial inner |
| | membrane; 0.3752 probability located in microbody |
| | (peroxisome); 0.3000 probability located in Golgi body |
| SignalP analysis: | Cleavage site between residues 45 and 46 |

A search of the NOV28a protein against the Geneseq database, a proprietary database that contains sequences published in patents and patent publication, yielded several homologous proteins shown in Table 28D.

TABLE 28D
Geneseq Results for NOV28a

| | | NOV28a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR13490 | Human C4 binding protein - | 13 . . . 218 | 190/208 (91%) | e−113 |
| | Homo sapiens, 581 aa. | 1 . . . 208 | 193/208 (92%) | |
| | [WO9111461-A, 08-AUG- | | | |
| | 1991] | | | |
| AAB57162 | Human prostate cancer | 62 . . . 170 | 107/109 (98%) | 1e−61 |
| | antigen protein sequence | 1 . . . 109 | 108/109 (98%) | |
| | SEQ ID NO: 1740 - Homo | | | |
| | sapiens, 110 aa. | | | |
| | [WO200055174-A1, 21-SEP- | | | |
| | 2000] | | | |
| AAW39924 | Amino acid sequence of a | 13 . . . 204 | 103/193 (53%) | 2e−57 |
| | mouse sperm protein | 1 . . . 192 | 132/193 (68%) | |
| | designated sp56 - Mus sp, | | | |
| | 579 aa. [WO9800440-A1, | | | |
| | 08-JAN-1998] | | | |
| AAG68150 | Codon modified human DAF | 32 . . . 217 | 74/191 (38%) | 3e−32 |
| | protein sequence SEQ ID | 22 . . . 212 | 106/191 (54%) | |
| | NO: 1 - Homo sapiens, 320 | | | |
| | aa. [JP2001211882-A, 07- | | | |
| | AUG-2001] | | | |
| ABB07542 | Amino acid sequence of | 45 . . . 217 | 68/177 (38%) | 2e−30 |
| | APT2334 - Synthetic, 271 aa. | 65 . . . 241 | 98/177 (54%) | |
| | [WO200204638-A1, 17- | | | |
| | JAN-2002] | | | |

In a BLAST search of public sequence datbases, the NOV28a protein was found to have homology to the proteins shown in the BLASTP data in Table 28E.

TABLE 28E
Public BLASTP Results for NOV28a

| | | NOV28a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P04003 | C4b-binding protein alpha | 1 . . . 218 | 202/220 (91%) | e−120 |
| | chain precursor (C4bp) | 5 . . . 224 | 205/220 (92%) | |
| | (Proline-rich protein) (PRP) - | | | |
| | Homo sapiens (Human), 597 | | | |
| | aa. | | | |
| Q28065 | C4b-binding protein alpha | 1 . . . 211 | 127/214 (59%) | 5e−71 |
| | chain precursor (C4bp) - Bos | 5 . . . 217 | 154/214 (71%) | |
| | taurus (Bovine), 610 aa. | | | |
| S53711 | C4BP alpha chain precursor - | 1 . . . 211 | 124/214 (57%) | 5e−68 |
| | rabbit, 597 aa. | 5 . . . 217 | 152/214 (70%) | |
| P08607 | C4b-binding protein precursor | 5 . . . 200 | 107/196 (54%) | 5e−59 |
| | (C4bp) - Mus musculus | 17 . . . 210 | 131/196 (66%) | |
| | (Mouse), 469 aa. | | | |
| Q91X48 | Complement component 4 | 5 . . . 200 | 107/196 (54%) | 8e−59 |
| | binding protein - Mus | 17 . . . 210 | 130/196 (65%) | |
| | musculus (Mouse), 469 aa. | | | |

PFam analysis predicts that the NOV28a protein contains the domains shown in Table 28F.

TABLE 28F
Domain Analysis of NOV28a

| | | Identities/ | |
|---|---|---|---|
| | | Similarities | |
| | NOV28a | for the Matched | |
| Pfam Domain | Match Region | Region | Expect Value |
| sushi | 46 . . . 104 | 16/68 (24%) | 1.3e−10 |
| | | 42/68 (62%) | |
| sushi | 109 . . . 166 | 20/64 (31%) | 6.2e−14 |
| | | 47/64 (73%) | |
| sushi | 171 . . . 216 | 20/64 (31%) | 0.012 |
| | | 38/64 (59%) | |

### Example 29

The NOV29 clone was analyzed, and the nucleotide and encoded polypeptide sequences are shown in Table 29A.

TABLE 29A
NOV29 Sequence Analysis

| | SEQ ID NO: 99 | 1356 bp | |
|---|---|---|---|
| NOV29a, | CTGCGCTCCCCAGGCGAGCTAACCGCCCGCTCGCCATGGGGAGCCCCGCACATCGGCCCG | | |
| CG171681-01 | | | |
| DNA Sequence | CGCTGCTGCTCCTGCTGCCGCCTCTGCTCCTGCTCCTGCTGCTGCGCGTCCCCCCCAGCC | | |
| | GCAGCTTCCCACATATGGAACCTCCTAGAATCAAGTGCCCAAGTGTGAAGGAACGCATTG | | |
| | CAGAACCCAACAAACTGACAGTCCGGGTGTCCTGGCAGACACCCGAAGGAAGACACACAG | | |
| | CAGATGGAATTCTTACTGATGTCATTCTAAAAGGCCTCCCCCCACGCTCCAACTTTCCAC | | |
| | AAGGAGACCACAAGATCCAGTACACAGTCTATCACAGAGCTGAGAATAAGGGCACTTGCA | | |
| | AATTTCCAGTTAAAGTAAGAGTCAAACCCTGTGGCAAACTCAATGCCCCAGAGAATCGTT | | |
| | ACATCAACTGCTCCAGCGACGGTGATAATTATGGAGCCACCTGTGAGTTCTCCTGCATCC | | |
| | GCGGCTATGACCTCCAGGGTAGCCCTCCCCGAGTATGTCAATCCAACCTGGCTTCGTCTG | | |
| | GCACGGACCCCACCTGTGCACCCATGAACCTCAATGTGGGTGTCACAACGGCAGCTGCAC | | |
| | TTCTGGATCAGTTTTATGAGAAAAGGACACTCCTCATTGTGTCCACACCCACAGCCCGAA | | |
| | ACCTCCTTTACCGGCTCCACCTAGGAATGCTGCAGCAAGCACAGTCTGGCCTTGATCTTC | | |
| | GACACATCACCGTGGTGGAGCTGGTGGGTGTGTTCCCGACTCTCATTGGCAGGATAGCAG | | |
| | CAAACATTATGCCTCCACCCCTAGCGCTGCAGCTCAGGCTGTTGCTGCCAATCCCACTCT | | |
| | ACTCCTTCAGTATGGTCCTACTGGATAAGCATGGCATGGACAAAGAGCGCTATGTCTCCC | | |
| | TGGTGATGCCTGTGGCCCTGTTCAACCTGATTGACACTTTTCCCTTGAGAAAAGAAGAGA | | |
| | TGGTCCTACAAGCCGAAATGACCCAGACCTGTAACACCTGACATGATGGTTCCTCTCTTG | | |
| | GCAATTCCTCTTCATTGTCTACATAGTGACATGCACACGGGAAAGCCTTAAAAATATCCT | | |
| | TGATGTACACATTTTATTTCTAATTTTAAAAGTCTATTTTATTATGAGCTTTCTTTGCAC | | |
| | TTAAAAATTAGCATCCTGCTTTTTGTACTTGGAAGTGTTTCAAAAAATTATATCACCATA | | |
| | TTTACTCTTTCTAACCTTTCTTTACTCCATCATGGCTGGTTGATTTGTAGAGAAATTAGA | | |
| | ACCCATAACCATACACAGGCTATCAACATGTTATTCAATGTCACACCTAACTCTTTTCTA | | |
| | TTTTGTTTTTTAAGTAAGACTTTTATTAATAAAACG | | |
| | ORF Start: ATG at 36 | | ORF Stop: TGA at 999 |
| | SEQ ID NO: 100 | 321 aa | MW at 35636.4kD |
| NOV29a, | MGSPAHRPALLLLLPPLLLLLLLRVPPSRSFPDMEPPRIKCPSVKERIAEPNKLTVRVSW | | |
| CG171681-01 | | | |
| Protein Sequence | ETPEGRDTADGILTDVILKGLPPCSNFPEGDHKIQYTVYDRAENKGTCKFRVKVRVKRCG | | |
| | KLNAPENGYMKCSSDGDNYGATCEFSCIGGYELQGSPARVCQSNLAWSGTEPTCAAINVN | | |
| | VGVRTAAALLDQFYEKRRLLIVSTPTARNLLYRLQLGMLQQAQCGLDLRHITVVELVGVF | | |
| | PTLICRICAKIMPPALALQLRLLLRIPLYSFSMVLVDKHGMDKERYVSLVMPVALFNLID | | |
| | TFPLRKEEMVLQAEMSQTCNT | | |
| | SEQ ID NO: 101 | 795 bp | |
| NOV29b, | CTTGGTCTCTTCGGTCTCCTGCCGCCCCCGGGAAGCGCGCTGCGCTGCCGAGGCGAGCTA | | |
| CG171681-03 | | | |
| DNA Sequence | AGCGCCCGCTCGCCATGCGGAGCCCCGCACATCGGCCCGCGCTGCTGCTGCTGCTGCCGC | | |
| | CTCTGCTGCTGCTGCTGCTGCGCGTCCCGCCCAGCCGCAGCTTCCCAGATACCCCGTGGT | | |
| | GCTCCCCCATCAAGGTGAAGTATGGGGATGTGTACTGCAGGGCCCCTCAAGGAGGATACT | | |
| | ACAAAACAGCCCTGGGAACCAGGTGCGACATTCGCTGCCAGAACGCCTACGAGCTGCATC | | |
| | GCTCTTCCCTACTGATCTGCCAGTCAAACAAACGATGGTCTGACAAGGTCATCTCCAAAC | | |
| | AAAAGCCATGTCCTACCCTTGCCATGCCAGCAAATGGACGGTTTAAGTGTGTAGATGGTG | | |
| | CCTACTTTAACTCCCGGTGTGAGTATTATTGTTCACCAGGATACACGTTGAAAGGGGAGC | | |
| | GGACCGTCACATGTATGGACAACAAGGCCTCCAGCGGCCGGCCAGCCTCCTGTGTGGATA | | |
| | TGGAACCTCCTAGAATCAAGTGCCCAAGTGTGAAGGAACGCATTGCACAACCCAACAAAC | | |
| | TGACAGTCCGGGTGTCCTGGGAGACACCCGAAGGAAGAGACACAGCAGATCCAATTCTTA | | |
| | CTGATGTCATTCTAAAAGGCCTCCCCCCAGGCTCCAACTTTCCAGAAGGAGACCACAAGA | | |
| | TCCAGTACACAGTCTATGACACAGCTGAGAATAAGGGCACTTGCAAATTTCGAGTTAAAG | | |
| | TAAGAGTCAAACGCTGTGGCAAACTCAATGCCCCAGAGAATGGTTACATGAAGTGCTCCA | | |
| | GCGACGGTGATAATTATGGAGCCACCTGTGAGTTCTCCTGCATCGGCGGCTATGAGCTCC | | |
| | AGGGTAGCCCTGCCCGAGTATGTCAATCCAACCTGGCTTGGTCTGGCACGGAGCCCACCT | | |
| | GTGCAGCCATCAACGTCAATGTGGGTGTCAGAACGGCAGCTGCACTTCTGGATCAGTTTT | | |
| | ATGAGAAAAGCAGACTCCTCATTGTGTCCACACCCACAGCCCGAAACCTCCTTTACCGGC | | |
| | TCCAGCTAGGAATGCTGCAGCAAGCACAGTGTCCCCTTGATCTTCGACACATCACCGTGG | | |
| | TGGAGCTGGTGGGTGTGTTCCCGACTCTCATTGGCAGGATAGGAGCAAAGATTATGCCTC | | |
| | CAGCCCTAGCGCTGCAGCTCAGGCTGTTGCTGCGAATCCCACTCTACTCCTTCAGTATGG | | |
| | TGCTAGTGGATAAGCATGGCATGGACAAAGAGCGCTATGTCTCCCTGGTGATCCCTGTGG | | |
| | CCCTGTTCAACCTCATTGACACTTTTCCCTTGAGAAAAGAAGAGATGGTCCTACAAGCCG | | |
| | AAATGAGCCAGACCTGTAACACCTGACATGATGGTTCCTCTCTTGGCAATTCCTCTTCAT | | |
| | TGTCTACATAGTGACATGCACACGGCAAAGCCTTAAAAATATCCTTGATGTACAGATTTT | | |
| | ATTTCTAATTTTAAAAGTCTATTTTATTATGAGCTTTCTTTGCACTTAAAAATTAGCATG | | |
| | CTGCTTTTTGTACTTGGAAGTGTTTCAAAAAATTATATGACCATATTTACTCTTTCTAAC | | |
| | TTTCTTTACTCCATCATGGCTGGTTGATTTTGTAGAGAAATTAGAACCCATAACCATACA | | |
| | CAGGCTATCAACATGTTATTCAATGTGACACCTAACTCTTTTCTATTTTGTTTTTTAAGT | | |
| | AAGACTTTTATTAATAAAACAAAATGTTTTGGAGCAAAAAAAAAAAAAAAAAAAA | | |
| | ORF Start: ATG at 75 | | ORF Stop: TGA at 1404 |
| | SEQ ID NO: 102 | 443 aa | MW at 49267.9kD |
| NOV29b, | MGSPAHRPALLLLLPPLLLLLLRVPPSRSFPDTPWCSPIKVKYGDVYCRAPQGCYYKTAL | | |
| CG171681-03 | | | |
| Protein Sequence | GTRCDIRCQKGYELHGSSLLTCQSNKRWSDKVICKQKRCPTLANPANGGFKCVDGAYFNS | | |
| | RCEYYCSPGYTLKCERTVTCMDNKAWSGRPASCVDMEPPRIKCPSVKERIAEPNKLTVRV | | |
| | SWETPEGRDTADGILTDVTLKGLPPGSNFPEGDHKIQYTVYDRAENKGTCKFRVKVRVKR | | |
| | CGKLNAPENGYMKCSSDGDNYGATCEFSCIGGYELQGSPARVCQSNLAWSGTEPTCAAMN | | |
| | VNVGVRTAAALLDQFYEKRRLLIVSTPTARNLLYRLQLCMLQQAQCGLDLRHITVVELVG | | |
| | VFPTLIGRIGAKIMPPALALQLRLLLRIPLYSFSMVLVDKHGMDKERYVSLVMPVALFNL | | |
| | IDTFPLRKEEMVLQAEMSQTCNT | | |
| | SEQ ID NO: 103 | 1798 bp | |
| NOV29c, | CTTGGTCTCTTCGGTCTCCTGCCGCCCCCCGGAAGCGCCCTCCGCTGCCGAGGCGAGCTA | | |
| CG171681-02 | | | |
| DNA Sequence | AGCCCCCGCTCGCCATGGGGAGCCCCGCACATCGGCCCGCGCTGCTGCTGCTGCTCCCGC | | |
| | CTCTGCTGCTGCTGCTGCTGCTGCGCGTCCCGCCCAGCCGCAGCTTCCCAGATACCCCGT | | |
| | GGTGCTCCCCCATCAAGGTGAAGTATGGGGATGTGTACTGCAGGGCCCCTCAAGGAGGAT | | |
| | ACTACAAAACAGCCCTGGGAACCAGGTGCGACATTCGCTGCCAGAAGGGCTACGAGCTGC | | |
| | ATGGCTCTTCCCTACTCATCTCCCACTCAAACAAACGATGGTCTCACAAGGTCATCTGCA | | |
| | AACAAAACCGATGTCCTACCCTTGCCATGCCAGCAAATGGAGGGTTTAAGTGTCTACATG | | |
| | GTGCCTACTTTAACTCCCGGTGTGACTATTATTCTTCACCAGGATACACGTTGAAAGGGG | | |
| | AGCGGACCGTCACATGTATGGACAACAAGGCCTGGAGCGGCCGGCCAGCCTCCTGTGTGG | | |
| | ATATGGAACCTCCTAGAATCAACTGCCCAAGTGTGAAGGAACGCATTGCAGAACCCAACA | | |
| | AACTGACAGTCCGGGTCTCCTCCGAGACACCCGAAGGAAGAGACACAGCAGATGGAATTC | | |
| | TTACTGATGTCATTCTAAAACCCCTCCCCCCACGCTCCAACTTTCCAGAAGCAGACCACA | | |
| | AGATCCAGTACACAGTCTATCACAGACCTCAGAATAAGGGCACTTGCAAATTTCGAGTTA | | |
| | AAGTAAGAGTCAAACCCTGTCGCAAACTCAATCCCCCAGAGAATGGTTACATGAAGTGCT | | |
| | CCAGCGACGCTGATAATTATGCAGCCACCTGTGAGTTCTCCTGCATCGGCGGCTATGAGC | | |
| | TCCAGGGTACCCCTGCCCGAGTATGTCAATCCAACCTGGCTTGGTCTGGCACCGAGCCCA | | |
| | CCTGTGCAGCCATGAACGTCAATGTGGGTGTCAGAACGGCAGCTCCACTTCTGGATCAGT | | |
| | TTTATGAGAAAAGCAGACTCCTCATTGTGTCCACACCCACAGCCCGAAACCTCCTTTACC | | |
| | GGCTCCAGCTAGGAATGCTGCAGCAAGCACAGTGTGGCCTTGATCTTCGACACATCACCC | | |
| | TGGTGGAGCTGGTGGGTGTGTTCCCGACTCTCATTGGCAGGATAGGAGCAAAGATTATGC | | |
| | CTCCAGCCCTAGCGCTGCAGCTCAGGCTGTTGCTGCGAATCCCACTCTACTCCTTCAGTA | | |
| | TGGTGCTAGTGGATAAGCATGGCATGGACAAAGAGCGCTATGTCTCCCTGGTGATGCCTC | | |
| | TGGCCCTGTTCAACCTGATTGACACTTTTCCCTTCACAAAAGAAGAGATGGTCCTACAAG | | |
| | CCGAAATGAGCCAGACCTGTAACACCTGACATGATGGTTCCTCTCTTGGCAATTCCTCTT | | |
| | CATTCTCTACATAGTGACATGCACACGGGAAAGCCTTAAAAATATCCTTGATGTACAGAT | | |
| | TTTATTTGTAATTTTAAAAGTCTATTTTATTATGAGCTTTCTTTGCACTTAAAAATTAGC | | |
| | ATGCTGCTTTTTGTACTTCGAAGTGTTTCAAAAAATTATATGACCATATTTACTCTTTCT | | |
| | AACTTTCTTTACTCCATCATGGCTGGTTGATTTTGTAGAGAAATTAGAACCCATAACCAT | | |
| | ACACAGGCTATCAACATGTTATTCAATGTGACACCTAACTCTTTTCTATTTTGTTTTTTA | | |
| | AGTAAGACTTTTATTAATAAAACAAAATGTTTTGGAGCAAAAAAAAAAAAAAAAAAAA | | |
| | ORF Start: ATG at 75 | | ORF Stop: TGA at 1407 |
| | SEQ ID NO: 104 | 444 aa | MW at 49381.1kD |

| | |
|---|---|
| NOV29c, | MGSPAHRPALLLLLPPLLLLLLLRVPPSRSFPDTPWCSPIKVKYGDVYCRAPQGGYYKTA |
| CG171681-02 | |
| Protein Sequence | LGTRCDIRCQKGYELHGSSLLICQSNKRWSDKVICKQKRCPTLAMPANGGFKCVDGAYFN |
| | SRCEYYCSPGYTLKGERTVTCMDNKAWSGRPASCVDMEPPRIKCPSVKERIAEPNKLTVR |
| | VSWETPEGRDTADGILTDVILKGLPPGSNFPEGDHKIQYTVYDRAENKGTCKFRVKVRVK |
| | RCGKLNAPENGYMKCSSDGDNYGATCEFSCIGGYELQGSPARVCQSNLAWSGTEPTCAAM |
| | NVNVGVRTAAALLDQFYEKRRLLIVSTPTARNLLYRLQLGMLQQAQCGLDLRHITVVELV |
| | GVPPTLIGRIGAKIMPPALALQLRLLLRIPLYSFSMVLVDKHGMDKERYVSLVMPVALFN |
| | LIDTFPLRKEEMVLQAEMSQTCNT |

Sequence comparison of the above protein sequences yields the following sequence relationships shown in Table 29B.

TABLE 29B
Comparison of NOV29a against NOV29b and NOV29c.

| | | Identities/ |
|---|---|---|
| | NOV29a Residues/ | Similarities for the |
| Protein Sequence | Match Residues | Matched Region |
| NOV29b | 33 . . . 321 | 273/289 (94%) |
| | 155 . . . 443 | 273/289 (94%) |
| NOV29c | 33 . . . 321 | 273/289 (94%) |
| | 156 . . . 444 | 273/289 (94%) |

Two polymorphic variants of NOV29c have been identified and are shown in Table 41K.

Further analysis of the NOV29a protein yielded the following properties shown in Table 29C.

TABLE 29C
Protein Sequence Properties NOV29a

| | |
|---|---|
| PSort analysis: | 0.8200 probability located in outside; |
| | 0.1000 probability located in endoplasmic reticulum |
| | (membrane); 0.1000 probability located in |
| | endoplasmic reticulum (lumen); 0.1000 probability |
| | located in lysosome (lumen) |
| SignalP analysis: | Cleavage site between residues 31 and 32 |

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB07747 | A human cancer-associated | 33 . . . 319 | 148/287 (51%) | 7e−89 |
| | protein-1 (CAP-1) - Homo | 178 . . . 464 | 205/287 (70%) | |
| | sapiens, 465 aa. | | | |
| | [WO200043508-A2, 27-JUL- | | | |
| | 2000] | | | |
| AAB59009 | Breast and ovarian cancer | 33 . . . 319 | 148/287 (51%) | 7e−89 |
| | associated antigen protein | 144 . . . 430 | 205/287 (70%) | |
| | sequence SEQ ID 717 - | | | |
| | Homo sapiens, 431 aa. | | | |
| | [WO200055173-A1, 21-SEP- | | | |
| | 2000] | | | |
| ABB72149 | Rat protein isolated from skin | 88 . . . 203 | 71/116 (61%) | 3e−38 |
| | cells SEQ ID NO: 188 - | 3 . . . 118 | 89/116 (76%) | |
| | Rattus sp, 118 aa. | | | |
| | [WO200190357-A1, 29- | | | |
| | NOV-2001] | | | |
| AAB55949 | Skin cell protein, SEQ ID | 88 . . . 203 | 71/116 (61%) | 3e−38 |
| | NO: 188 - Rattus sp, 118 aa. | 3 . . . 118 | 89/116 (76%) | |
| | [WO200069884-A2, 23- | | | |
| | NOV-2000] | | | |
| AAY76010 | Rat DRS protein homolog, | 88 . . . 203 | 71/116 (61%) | 3e−38 |
| | SEQ ID NO: 188 - Rattus sp, | 3 . . . 118 | 89/116 (76%) | |
| | 118 aa. [WO9955865-A1, | | | |
| | 04-NOV-1999] | | | |

In a BLAST search of public sequence datbases, the NOV29a protein was found to have homology to the proteins shown in the BLASTP data in Table 29E.

TABLE 29E
Public BLASTP Results for NOV29a

| | | NOV29a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P78539 | Sushi repeat-containing | 33 . . . 321 | 289/289 (100%) | e−168 |
| | protein SRPX precursor - | 176 . . . 464 | 289/289 (100%) | |
| | Homo sapiens (Human), 464 | | | |
| | aa. | | | |
| Q63769 | Sushi repeat-containing | 33 . . . 321 | 279/289 (96%) | e−164 |
| | protein SRPX precursor | 176 . . . 464 | 286/289 (98%) | |
| | (DRS protein) (Down- | | | |
| | regulated by V-SRC) - | | | |
| | Rattus norvegicus (Rat), 464 | | | |
| | aa. | | | |
| Q9R0 m3 | Sushi-repeat-containing | 33 . . . 320 | 276/288 (95%) | e−163 |
| | protein - Mus musculus | 176 . . . 463 | 285/288 (98%) | |
| | (Mouse), 464 aa. | | | |
| Q9R0 m2 | Sushi-repeat-containing | 33 . . . 320 | 276/288 (95%) | e−163 |
| | protein - Mus musculus | 92 . . . 379 | 285/288 (98%) | |
| | (Mouse), 380 aa. | | | |
| AAM73690 | Sushi-repeat containing | 33 . . . 319 | 152/287 (52%) | 2e−89 |
| | protein - Mus musculus | 123 . . . 409 | 203/287 (69%) | |
| | (Mouse), 410 aa (fragment). | | | |

PFam analysis predicts that the NOV29a protein contains the domains shown in Table 29F.

TABLE 29F
Domain Analysis of NOV29a

| | | Identities/ | |
|---|---|---|---|
| | | Similarities | |
| | NOV29a | for the Matched | |
| Pfam Domain | Match Region | Region | Expect Value |
| HYR | 33 . . . 114 | 27/86 (31%) | 2.2e−34 |
| | | 78/86 (91%) | |
| sushi | 119 . . . 174 | 19/64 (30%) | 2.7e−09 |
| | | 41/64 (64%) | |

### Example 30

The NOV30 clone was analyzed, and the nucleotide and encoded polypeptide sequences are shown in Table 30A.

TABLE 30A
NOV30 Sequence Analysis

| | SEQ ID NO: 105 | 1499 bp | |
|---|---|---|---|
| NOV30a, | ACGCGTGTAGGTGGCCCAGGCAAATAGTGTCATCGATTGGCCTATGTCGTTATGGTGGGA | | |
| CG51117-01 | | | |
| DNA Sequence | GGATTGACTGCTGCTGGGGCTGGGCTCGCCAGTCTTGGGGACAGTGTCAGCCTTTCTACG | | |
| | TCTTAAGGCAGAGAATAGCCAGGATAAGGTGCCAGCTCAAAGCTGTGTGCCAACCACGAT | | |
| | GCAAACATGGTGAATGTATCGGGCCAAACAAGTGCAAGTGTCATCCTGGTTATGCTGGAA | | |
| | AAACCTGGTATTCAAGTTTTAAATGAGTGTGGCCTGAAGCCCCGGCCCTGTAAGCACAGG | | |
| | TGCATGAACACTTACGGCAGCTACAAGTGCTACTGTCTCAACGGATATATGCTCATGCCG | | |
| | GATGGTTCCTGCTCAAGTGCCCTGACCTGCTCCATGGCAAACTGTCAGTATGGCTGTGAT | | |
| | GTTGTTAAAGGACAAATACGGTGCCAGTGCCCATCCCCTGGCCTGCAGCTGGCTCCTGAT | | |
| | GGGAGGACCTGTGTAGATGTTGATGAATGTGCTACAGGAAGAGCCTCCTGCCCTAGATTT | | |
| | AGGCAATGTGTCAACACTTTTGGGAGCTACATCTGCAAGTGTCATAAAGGCTTCGATCTC | | |
| | ATGTATATTGGAGGCAAATATCAATGTCATGACATAGACGAATGCTCACTTGGTCAGTAT | | |
| | CAGTGCAGCAGCTTTGCTCGATGTTATAACGTACGTGGGTCCTACAAGTGCAAATGTAAA | | |
| | GAAGGATACCAGGGTGATGGACTGACTTGTGTGTATATCCCAAAAGTTATGATTGAACCT | | |
| | TCAGGTCCAATTCATGTACCAAAGGGAAATGGTACCATTTTAAAGGGTGACACAGGAAAT | | |
| | AATAATTGGATTCCTGATGTTGGAAGTACTTGGTGGCCTCCGAAGACACCATATATTCCT | | |
| | CCTATCATTACCAACAGGCCTACTTCTAAGCCAACAACAAGACCTACACCAAAGCCAACA | | |
| | CCAATTCCTACTCCACCACCACCACCACCCCTGCCAACAGAGCTCAGAACACCTCTACCA | | |
| | CCTACAACCCCAGAAAGGCCAACCACCGGACTGACAACTATAGCACCAGCTGCCAGTACA | | |
| | GATGTGTTCATTCCACGGCAACCTTCAAATGACTTGTTTGAAATATTTGAAATAGAAAGA | | |
| | GGAGTCAGTGCAGACGATGAAGCAAAGGATGATCCAGGTGTTCTGGTACACAGTTGTAAT | | |
| | TTTGACCATGGACTTTGTGGATGGATCAGGGAGAAAGACAATGACTTGCACTGGGAACCA | | |
| | ATCAGGGACCCAGCAGGTGGACAATATCTGACAGTGTCGGCAGCCAAAGCCCCAGGGGGA | | |
| | AAAGCTGCACGCTTGGTGCTACCTCTCGGCCGCCTCATGCATTCAGGGGACCTGTGCCTG | | |
| | TCATTCAGGCACAAGGTGACGGGGCTGCACTCTGGCACACTCCAGGTGTTTGTGAGAAA | | |
| | ORF Start: at 148 | | ORF Stop: at 1498 |
| | SEQ ID NO: 106 | 450 aa | MW at 48855.5kD |
| NOV30a, | GASSKLCANHDANMVNVSGQTSASVILVMLEKPGIQVLNECGLKPRPCKHRCMNTYGSYK | | |
| CG51117-01 | | | |
| Protein Sequence | CYCLNGYMLMPDGSCSSALTCSMANCQYGCDVVKGQIRCQCPSPGLQLAPDGRTCVDVDE | | |
| | CATGRASCPRFRQCVNTFGSYICKCHKGFDLMYIGGKYQCHDIDECSLGQYQCSSFARCY | | |
| | NVRGSYKCKCKEGYQGDGLTCVYIPKVMIEPSGPIHVPKGNGTILKGDTGNNNWIPDVGS | | |
| | TWWPPKTPYIPPIITNRPTSKPTTRPTPKPTPIPTPPPPPPLPTELRTPLPPTTPERPTT | | |
| | GLTTIAPAASTPPGGITVDNRVQTDPQKPRGDVFIPRQPSNDLFEIFEIERGVSADDEAK | | |
| | DDPGVLVHSCNFDHGLCGWIREKDNDLHWEPIRDPAGGQYLTVSAAKAPGGKAARLVLPL | | |
| | GRLMHSGDLCLSFRHKVTGLHSGTLQVFVR | | |
| | SEQ ID NO: 107 | 1638 bp | |
| NOV30b, | GAGTTCGACGGGAGGTGGCCCAGGCAAATAGTGTCATCGATTGGCCTATGTCGTTATGGT | | |
| CG51117-05 | | | |
| DNA Sequence | GGGAGGATTGACTGCTGCTGGGGCTGGGCTCGCCAGTCTTGGGGACAGTGTCAGCCTGTG | | |
| | TGCCAACCACGATGCAAACATGGTGAATGTATCGGGCCAAACAAGTGCAAGTGTCATCCT | | |
| | GGTTATGCTGGAAAAACCTGTATTCAAGTTTTAAATGAGTGTGGCCTGAAGCCCCGGCCC | | |
| | TGTAAGCACAGGTGCATGAACACTTACGGCAGCTACAAGTGCTACTGTCTCAACGGATAT | | |
| | ATGCTCATGCCGGATGGTTCCTGCTCAAGTGCCCTGACCTGCTCCATGGCAAACTGTCAG | | |
| | TATGGCTGTGATGTTGTTAAAGGACAAATACGGTGCCAGTGCCCATCCCCTGGCCTGCAG | | |
| | CTGGCTCCTGATGGGAGGACCTGTGTAGATGTTGATGAATGTGCTACAGGAAGAGCCTCC | | |
| | TGCCCTAGATTTAGGCAATGTGTCAACACTTTTGGGAGCTACATCTGCAAGTGTCATAAA | | |
| | GGCTTCGATCTCATGTATATTGGAGGCAAATATCAATGTCATGACATAGACGAATGCTCA | | |
| | CTTGGTCAGTATCAGTGCAGCAGCTTTGCTCGATGTTATAACGTACGTGGGTCCTACAAG | | |
| | TGCAAATGTAAAGAAGGATACCAGGGTGATGGACTGACTTGTGTGTATATCCCAAAAGTT | | |
| | ATGATTGAACCTTCAGGTCCAATTCATGTACCAAAGGGAAATGGTACCATTTTAAAGGGT | | |
| | GACACAGGAAATAATAATTGGATTCCTGATGTTGGAAGTACTTGGTGGCCTCCGAAGACA | | |
| | CCATATATTCCTCCTATCATTACCAACAGGCCTACTTCTAAGCCAACAACAAGACCTACA | | |
| | CCAAAGCCAACACCAATTCCTACTCCACCACCACCACCACCCCTGCCAACAGAGCTCAGA | | |
| | ACACCTCTACCACCTACAACCCCAGAAAGGCCAACCACCGGACTGACAACTATAGCACCA | | |
| | GCTGCCAGTACACCTCCAGGAGGGATTACAGTTGACAACAGGGTACAGACAGACCCTCAG | | |
| | AAACCCAGAGGAGATGTGTTCATTCCACGGCAACCTTCAAATGACTTGTTTGAAATATTT | | |
| | GAAATAGAAAGAGGAGTCAGTGCAGACGATGAAGCAAAGGATGATCCAGGTGTTCTGGTA | | |
| | CACAGTTGTAATTTTGACCATGGACTTTGTGGATGGATCAGGGAGAAAGACAATGACTTG | | |
| | CACTGGGAACCAATCAGGGACCCAGCAGGTGGACAATATCTGACAGTGTCGGCAGCCAAA | | |
| | GCCCCAGGGGGAAAAGCTGCACGCTTGGTGCTACCTCTCGGCCGCCTTATGCATTCAGGG | | |
| | GACCTGTGCCTGTCATTCAGGCACAAGGTGACGGGGCTGCACTCTGGCACACTCCAGGTG | | |
| | TTTGTGAGAAAACACGGTGCCCACGGAGCAGCCCTGTGGGGAAGAAATGGTGGCCATGGC | | |
| | TGGAGGCAAACACAGATCACCTTGCGAGGGGCTGACATCAAGAGCGTCGTCTTCAAAGGT | | |
| | GAAAAAAGGCGTGGTCACACTGGGGAGATTGGATTAGATGATGTGAGCTTGAAAAAAGGC | | |
| | CACTGCTCTGAAGAACGC | | |
| | ORF Start: at 1 | | ORF Stop: end of sequence |
| | SEQ ID NO: 108 | 546 aa | MW at 59854.9kD |
| NOV30b, | EFDGRWPRQIVSSIGLCRYGGRIDCCWGWARQSWGQCQPVCQPRCKHGECIGPNKCKCHP | | |
| CG51117-05 | | | |
| Protein Sequence | GYAGKTCIQVLNECGLKPRPCKHRCMNTYGSYKCYCLNGYMLMPDGSCSSALTCSMANCQ | | |
| | YGCDVVKGQIRCQCPSPGLQLAPDGRTCVDVDECATGRASCPRFRQCVNTFGSYICKCHK | | |
| | GFDLMYIGGKYQCHDIDECSLGQYQCSSFARCYNVRGSYKCKCKEGYQGDGLTCVYIPKV | | |
| | MIEPSGPIHVPKGNGTILKGDTGNNNWIPDVGSTWWPPKTPYIPPIITNRPTSKPTTRPT | | |
| | PKPTPIPTPPPPPPLPTELRTPLPPTTPERPTTGLTTIAPASSTPPGGITVDNRVQTDPQ | | |
| | KPRGDVRIPRQPSNDLFEIFEIERGVSADDEAKDDPGVLVHSCNFDHGLCGWIREKDNDL | | |
| | HWEPIRDPAGGQYLTVSAAKAPGGKAARLVLPLGRLMHSGDLCLSFRHKVTGLHSGTLQV | | |
| | FVRKHGAHGAALWGRNGGHGWRQTQITLRGADIKSVVFKGEKRRGHTGEIGLDDVSLKKG | | |
| | HCSEER | | |
| | SEQ ID NO: 109 | 2245 bp | |
| NOV30c, | GGACACTGACATGGACTGAAGGAGTAGAAAAGAAGGGAGCGGGAGGGGGCTCCGGGCGCC | | |
| CG51117-06 | | | |
| DNA Sequence | GCGCAGCAGACCTGCTCCGGCCGCGCGCCTCGCCGCTGTCCTCCGGGAGCGGCAGCAGTA | | |
| | GCCCGGGCGGCGAGGGCTGGGGGTTCCTCGAGACTCTCAGAGGGGCGCCTCCCATCGGCG | | |
| | CCCACCACCCCAACCTGTTCCTCGCGCGCCACTGCGCTGCGCCCCAGGACCCGCTGCCCA | | |
| | ACATGGATTTTCTCCTGGCGCTGGTGCTGGTATCCTCGCTCTACCTGCAGGCGGCCGCCG | | |
| | AGTTCGACGGGAGTAGGTGGCCCAGGCAAATAGTGTCATCGATTGGCCTATGTCGTTATG | | |
| | GTGGGAGGATTGACTGCTGCTGGGGCTGGGCTCGCCAGTCTTGGGGACAGTGTCAGCCTT | | |
| | TCTACGTCTTAAGGCAGAGAATAGCCAGGATAAGGTGCCAGCTCAAAGCTGTGTGCCAAC | | |
| | CACGATGCAAACATGGTGAATGTATCGGGCCAAACAAGTGCAAGTGTCATCCTGGTTATG | | |
| | AGCCTCTTTTCCAACCCCTGGATCACCAAGCCACAAGTTTGCCTTCAAGGGATCTAAATG | | |
| | AGTGTGGCCTGAAGCCCCGGCCCTGTAAGCACAGGTGCATGAACACTTACGGCAGCTACA | | |
| | AGTGCTACTGTCTCAACGGATATATGCTCATGCCGGATGGTTCCTGCTCAAGTGCCCTGA | | |
| | CCTGCTCCATGGCAAACTGTCAGTATGGCTGTGATGTTGTTAAAGGACAAATACGGTGCC | | |
| | AGTGCCCATCCCCTGGCCTGCAGCTGGCTCCTGATGGGAGGACCTGTGTAGATGTTGATG | | |
| | AATGTGCTACAGGAAGAGCCTCCTGCCCTAGATTTAGGCAATGTGTCAACACTTTTGGGA | | |
| | GCTACATCTGCAAGTGTCATAAAGGCTTCCATCTCATGTATATTGGAGGCAAATATCAAT | | |
| | GTCATGACATAGACGAATGCTCACTTGGTCAGTATCAGTGCACCACCTTTCCTCGATGTT | | |
| | ATAACATACGTGGGTCCTACAACTGCAAATGTAAAGAACGATACCAGGGTGATGGACTGA | | |
| | CTTGTGTGTATATCCCAAAAGTTATGATTGAACCTTCAGGTCCAATTCATGTACCAAAGG | | |
| | GAAATGGTACCATTTTAAAGGGTGACACAGGAAATAATAATTGGATTCCTGATGTTGGAA | | |
| | GTACTTGGTGGCCTCCGAAGACACCATATATTCCTCCTATCATTACCAACAGGCCTACTT | | |
| | CTAAGCCAACAACAAGACCTACACCAAAGCCAACACCAATTCCTACTCCACCACCACCAC | | |
| | CACCCCTGCCAACAGACCTCAGAACACCTCTACCACCTACAACCCCAGAAAGGCCAACCA | | |
| | CCGGACTGACAACTATAGCACCAGCTGCCAGTACACCTCCAGGAGGGATTACAGTTGACA | | |
| | ACAGGGTACAGACAGACCCTCAGAAACCCAGAGGAGATGTGTTCATTCCACGGCAACCTT | | |
| | CAAATGACTTGTTTGAAATATTTGAAATAGAAAGAGGAGTCAGTGCAGACGATGAAGCAA | | |
| | AGGATGATCCAGGTGTTCTGGTACACAGTTGTAATTTTGACCATGGACTTTGTGGATGGA | | |
| | TCAGGGAGAAAGACAATGACTTGCACTGGGAACCAATCAGGGACCCAGCAGGTGGACAAT | | |
| | ATCTGACAGTGTCGGCAGCCAAAGCCCCAGGGGGAAAAGCTGCACGCTTGGTGCTACCTC | | |
| | TCGGCCGCCTTATGCATTCAGGGGACCTGTGCCTGTCATTCAGGCACAAGGTGACGGGGC | | |
| | TGCACTCTGGCACACTCCAGGTCTTTGTGAGAAAACACCCTGCCCACGGAGCAGCCCTGT | | |
| | GGGGAAGAAATGGTCGCCATGGCTGCAGGCAAACACAGATCACCTTGCCAGGGGCTGACA | | |
| | TCAAGAGCGTCGTCTTCAAAGGTGAAAAAAGCCCTGGTCACACTCGGGAGATTGGATTAG | | |
| | ATGATGTGAGCTTGAAAAAAGGCCACTGCTCTGAAGAACGCTAACAACTCCAGAACTAAC | | |
| | AATGAACTCCTATGTTGCTCTATCCTCTTTTTCCAATTCTCATCTTCTCTCCTCTTCTCC | | |
| | CTTTTATCAGGCCTAGGAGAAGAGTGGGTCAGTGGGTCAGAAGGAAGTCTATTTGGTGAC | | |
| | CCAGGTTCTTCTGGCCTGCTTTTGT | | |
| | ORF Start: ATG at 243 | | ORF Stop: TAA at 2082 |
| | SEQ ID NO: 110 | 613 aa | MW at 67416.5kD |
| NOV30c, | MDFLLALVLVSSLYLQAAAEFDGSRWPRQIVSSIGLCRYGGRIDCCWGWARQSWGQCQPF | | |
| CG51117-06 | | | |
| Protein Sequence | YVLRQRIARIRCQLKAVCQPRCKHGECIGPNKCKCHPGYAGKTCNQDEHIPAPLDQGSEQ | | |
| | PLFQPLDHQATSLPSRDLNECGLKPRPCKHRCMNTYGSYKCYCLNGYMLMPDGSCSSALT | | |
| | CSMANCQYGCDVVKGQIRCQCPSPGLQLAPDGRTCVDVDECATGPASCPRFRQCVNTFGS | | |
| | YICKCHKGFDLMYIGGKYQCHDIDECSLGQYQCSSFARCYNIRGSYKCKCKEGYQCDGLT | | |
| | CVYIPKVMIEPSGPIHVPKGNGTILKCDTGNNNWIPDVGSTWWPPKTPYIPPIITNRPTS | | |
| | KPTTRPTPKPTPIPTPPPPPPLPTELRTPLPPTTPERPTTGLTTIAPAASTPPGGITVDN | | |
| | RVQTDPQKPRGDVFIPRQPSNDLFEIFEIERGVSADDEAKDDPGVLVHSCNFDHGLCGWI | | |
| | REKDNDLHWEPIRDPAGGQYLTVSAAKAPGGKAARLVLPLGRLMHSGDLCLSFRHKVTGL | | |
| | HSGTLQVFVRKHGAHGAALWGRNGGHGWRQTQITLRGADIKSVVFKGEKRRGHTGEIGLD | | |
| | DVSLKKGHCSEER | | |
| | SEQ ID NO: 111 | 2194 bp | |
| NOV30d, | GGACACTGACATGGACTGAAGGAGTAGAAAAGAAGGGACCGGGAGGGGGCTCCGGGCGCC | | |
| CG51117-07 | | | |
| DNA Sequence | GCGCAGCAGACCTGCTCCGGCCCCGCGCCTCGCCGCTGTCCTCCGGGAGCGGCAGCAGTA | | |
| | GCCCGGGCGGCGACGCCTGGGGGTTCCTCGAGACTCTCAGAGGGGCGCCTCCCATCGGCG | | |
| | CCCACCACCCCAACCTGTTCCTCGCGCGCCACTGCGCTGCGCCCCAGGACCCGCTGCCCA | | |
| | ACATGGATTTTCTCCTGGCGCTGGTGCTGCTATCCTCGCTCTACCTGCAGGCGGCCGCCG | | |
| | AGTTCGACGGCAGTAGGTGGCCCAGGCAAATAGTGTCATCGATTGCCCTATGTCGTTATG | | |
| | GTGGGAGGATTGACTGCTGCTGGGGCTGGGCTCGCCAGTCTTGGGGACAGTGTCAGCCTG | | |
| | TGTGCCAACCACGATGCAAACATGGTGAATGTATCGGGCCAAACAAGTGCAAGTGTCATC | | |
| | CTGGTTATGCTGGAAAAACCTGTAATCAAGACGAGCACATCCCAGCTCCTCTTGACCAAG | | |
| | GCAGTGAACAGCCTCTTTTCCAACCCCTGGATCACCAAGCCACAAGTTTGCCTTCAAGGG | | |
| | ATCTAAATGAGTGTGGCCTGAAGCCCCGGCCCTGTAAGCACAGGTGCATGAACACTTACG | | |
| | GCAGCTACAAGTGCTACTGTCTCAACGGATATATGCTCATGCCGGATGGTTCCTCCTCAA | | |
| | GTGCCCTGACCTGCTCCATGGCAAACTGTCAGTATGGCTGTGATGTTGTTAAAGGACAAA | | |
| | TACGGTGCCACTGCCCATCCCCTCGCCTGCAGCTCGCTCCTGATGGGAGGACCTGTGTAG | | |
| | ATGTTCATCAATCTCCTACAGGAAGAGCCTCCTGCCCTAGATTTACGCAATGTGTCAACA | | |
| | CTTTTGGGAGCTACATCTGCAACTGTCATAAAGGCTTCGATCTCATGTATATTCGACCCA | | |
| | AATATCAATCTCATCACATACACGAATGCTCACTTGGTCAGTATCAGTCCAGCACCTTTG | | |
| | CTCGATGTTATAACATACCTGCGTCCTACAAGTGCAAATGTAAACAAGGATACCAGCGTG | | |
| | ATGGACTGACTTGTGTGTATATCCCAAAAGTTATCATTCAACCTTCACGTCCAATTCATG | | |
| | TACCAAAGGGAAATGGTACCATTTTAAAGGGTGACACACCAAATAATAATTGCATTCCTG | | |
| | ATGTTGGAAGTACTTGGTGGCCTCCGAAGACACCATATATTCCTCCTATCATTACCAACA | | |
| | GGCCTACTTCTAAGCCAACAACAAGACCTACACCAAAGCCAACACCAATTCCTACTCCAC | | |
| | CACCACCACCACCCCTCCCAACACAGCTCAGAACACCTCTACCACCTACAACCCCAGAAA | | |
| | GGCCAACCACCGGACTGACAACTATAGCACCAGCTGCCAGTACACCTCCAGGAGGGATTA | | |
| | CAGTTGACAACAGGGTACAGACAGACCCTCAGAAACCCAGAGCAGATGTGTTCATTCCAC | | |
| | GGCAACCTTCAAATGACTTGTTTGAAATATTTGAAATAGAAAGACGAGTCAGTGCAGACG | | |
| | ATGAAGCAAAGGATGATCCAGGTGTTCTCGTACACACTTGTAATTTTGACCATGGACTTT | | |
| | GTGGATGGATCAGGGAGAAAGACAATGACTTCCACTGGCAACCAATCACGGACCCAGCAG | | |
| | GTGGACAATATCTGACAGTGTCGGCAGCCAAAGCCCCAGGGGGAAAAGCTGCACGCTTGG | | |
| | TGCTACCTCTCGGCCGCCTTATGCATTCAGGGGACCTCTCCCTGTCATTCACGCACAAGG | | |
| | TGACGCGGCTGCACTCTGGCACACTCCAGGTGTTTGTCACAAAACACGGTGCCCACGGAG | | |
| | CAGCCCTGTGGGGAAGAAATGGTGGCCATGGCTGGAGGCAAACACAGATCACCTTGCGAG | | |
| | GGGCTGACATCAAGAGCGTCGTCTTCAAAGGTGAAAAAAGGCGTGGTCACACTGGGGAGA | | |
| | TTGGATTAGATGATGTGAGCTTGAAAAAAGGCCACTGCTCTGAAGAACGCTAACAACTCC | | |
| | AGAACTAACAATCAACTCCTATGTTGCTCTATCCTCTTTTTCCAATTCTCATCTTCTCTC | | |
| | CTCTTCTCCCTTTTATCAGGCCTAGGAGAAGAGTGGGTCAGTGGGTCAGAAGGAAGTCTA | | |
| | TTTGGTGACCCAGGTTCTTCTGGCCTGCTTTTGT | | |
| | ORF Start: ATG at 243 | | ORF Stop: TAA at 2031 |
| | SEQ ID NO: 112 | 596 aa | MW at 65299.9kD |
| NOV30d, | MDFLLALVLVSSLYLQAAAEFDGSRWPRQIVSSIGLCRYGGRIDCCWGWARQSWGQCQPV | | |
| CG51117-07 | | | |
| Protein Sequence | CQPRCKHGECIGPNKCKCHPGYAGKTCNQDEHIPAPLDQGSEQPLFQPLDHQATSLPSRD | | |
| | LNECGLKPRPCKHRCMNTYGSYKCYCLNGYMLMPDGSCSSALTCSMANCQYGCDVVKGQI | | |
| | RCQCPSPGLQLAPDGRTCVDVDECATGRASCPRFRQCVNTFGSYICKCHKGFDLMYIGGK | | |
| | YQCHDIDECSLGQYQCSSFARCYNIRGSYKCKCKEGYQGDGLTCVYIPKVMIEPSGPIHV | | |
| | PKGNGTILKGDTGNNNWIPDVGSTWWPPKTPYIPPIITNRPTSKPTTRPTPKPTPIPTPP | | |
| | PPPPLPTELRTPLPPTTPERPTTGLTTIAPAASTPPGGITVDNRVQTDPQKPRGDVFIPR | | |
| | QPSNDLFEIFEIERGVSADDEAKDDPGVLVHSCNFDHGLCGWIREKDNDLHWEPIRDPAG | | |
| | GQYLTVSAAKAPGGKAARLVLPLGRLMHSGDLCLSFRHKVTGLHSGTLQVFVRKHGAHGA | | |
| | ALWGRNGGHGWRQTQITLRGADIKSVVFKGEKRRGHTGEIGLDDVSLKKGHCSEER | | |
| | SEQ ID NO: 113 | 2112 bp | |
| NOV30e, | GGGAGGGGGCTCCGGGCGCCGCGCAGCAGACCTGCTCCCGCCCCGCGCCTCGCCGCTGTC | | |
| CG51117-03 | | | |
| DNA Sequence | CTCCGGGAGCGGCAGCAGTAGCCCGGGCGGCGAGGGCTGGGCGTTCCTCGAGACTCTCAG | | |
| | AGGGGCGCCTCCCATCGGCGCCCACCACCCCAACCTGTTCCTCGCGCGCCACTGCGCTGC | | |
| | GCCCCAGGACCCGCTGCCCAACATGGATTTTCTCCTGGCGCTGGTCCTGGTATCCTCGCT | | |
| | CTACCTGCACGCGGCCGCCGAGTTCGACCCGAGGTGGCCCAGGCAAATAGTGTCATCGAT | | |
| | TGGCCTATGTCGTTATGGTGGGAGGATTGACTGCTGCTGGGGCTGGGCTCGCCAGTCTTG | | |
| | GGGACAGTGTCAGCCTTTCTACGTCTTAAGGCAGAGAATAGCCAGCATAAGGTGCCAGCT | | |
| | CAAACCTGTGTGCCAACCACGATCCAAACATGGTGAATGTATCGGCCCAAACAAGTGCAA | | |
| | GTGTCATCCTGGTTATCCTGGAAAAACCTGTATTCAAGTTTTAAATGAGTGTGGCCTGAA | | |
| | GCCCCGGCCCTGTAACCACAGGTGCATGAACACTTACGGCACCTACAAGTGCTACTGTCT | | |
| | CAACGGATATATGCTCATGCCGGATGGTTCCTGCTCAAGTGCCCTGACCTGCTCCATGGC | | |
| | AAACTGTCAGTATGGCTGTGATGTTGTTAAAGGACAAATACGGTGCCAGTGCCCATCCCC | | |
| | TGGCCTGCAGCTGGCTCCTGATGGGAGGACCTGTCTAGATGTTGATGAATGTGCTACAGG | | |
| | AAGAGCCTCCTGCCCTAGATTTAGGCAATGTCTCAACACTTTTGGGACCTACATCTGCAA | | |
| | GTGTCATAAAGGCTTCGATCTCATGTATATTCGAGGCAAATATCAATCTCATGACATAGA | | |
| | CGAATGCTCACTTGGTCAGTATCAGTGCAGCAGCTTTGCTCGATGTTATAACGTACGTGG | | |
| | GTCCTACAAGTGCAAATGTAAAGAAGGATACCAGGGTGATGGACTGACTTGTGTGTATAT | | |
| | CCCAAAAGTTATGATTGAACCTTCAGGTCCAATTCATGTACCAAAGGGAAATGGTACCAT | | |
| | TTTAAAGGGTGACACAGGAAATAATAATTGGATTCCTGATGTTGGAAGTACTTGGTGGCC | | |
| | TCCGAAGACACCATATATTCCTCCTATCATTACCAACAGGCCTACTTCTAAGCCAACAAC | | |
| | AAGACCTACACCAAAGCCAACACCAATTCCTACTCCACCACCACCACCACCCCTGCCAAC | | |
| | AGAGCTCAGAACACCTCTACCACCTACAACCCCAGAAAGCCCAACCACCGGACTGACAAC | | |
| | TATAGCACCAGCTGCCAGTACACCTCCAGGACCGATTACAGTTGACAACACGGTACAGAC | | |
| | AGACCCTCAGAAACCCAGACCAGATGTGTTCATTCCACGGCAACCTTCAAATGACTTGTT | | |
| | TGAAATATTTGAAATAGAAACAGGAGTCAGTGCAGACGATCAACCAAAGGATGATCCAGG | | |
| | TGTTCTCCTACACACTTGTAATTTTGACCATGGACTTTCTGGATGGATCAGGGAGAAAGA | | |
| | CAATGACTTGCACTGGGAACCAATCAGCGACCCAGCAGGTGCACAATATCTGACAGTGTC | | |
| | GGCAGCCAAAGCCCCACGGGGAAAACCTGCACGCTTGGTCCTACCTCTCGGCCGCCTTAT | | |
| | GCATTCAGGGGACCTGTGCCTGTCATTCAGGCACAAGGTCACGCCCCTGCACTCTGGCAC | | |
| | ACTCCAGGTGTTTGTGAGAAAACACGGTCCCCACGGAGCAGCCCTGTGGCGAAGAAATGG | | |
| | TGGCCATGGCTGGAGCCAAACACAGATCACCTTCCGAGGGGCTCACATCAAGAGCGTCGT | | |
| | CTTCAAAGGTCAAAAAAGGCGTGGTCACACTGCGGAGATTCGATTAGATGATGTGAGCTT | | |
| | GAAAAAAGGCCACTGCTCTGAAGAACGCTAACAACTCCAGAACTAACAATGAACTCCTAT | | |
| | GTTGCTCTATCCTCTTTTTCCAATTCTCATCTTCTCTCCTCTTCTCCCTTTTATCAGGCC | | |
| | TAGGAGAAGACTGGGTCAGTGGGTCAGAACGAAGTCTATTTGGTGACCCAGGTTCTTCTG | | |
| | GCCTGCTTTTGT | | |
| | ORF Start: ATG at 203 | | ORF Stop: TAA at 1949 |
| | SEQ ID NO: 114 | 582 aa | MW at 63991.9kD |
| NOV30e, | MDFLLALVLVSSLYLQAAAEFDCRWPRQIVSSIGLCRYGCRIDCCWGWARQSWGQCQPFY | | |
| CG51117-03 | | | |
| Protein Sequence | VLRQRIARIRCQLKAVCQPRCKHGECIGPNKCKCHPGYAGKTCIQVLNECGLKPRPCKHR | | |
| | CMNTYGSYKCYCLNGYMLMPDGSCSSALTCSMANCQYGCDVVKGQIRCQCPSPGLQLAPD | | |
| | GRTCVDVDECATGRASCPRFRQCVNTFGSYICKCHKGFDLMYIGGKYQCHDIDECSLGQY | | |
| | QCSSFARCYNVRGSYKCKCKEGYQGDGLTCVYIPKVMIEPSGPIHVPKGNGTILKGDTGN | | |
| | NNWIPDVGSTWWPPKTPYIPPIITNRPTSKPTTRPTPKPTPIPTPPPPPPLPTELRTPLP | | |
| | PTTPERPTTGLTTIAPAASTPPGGITVDNRVQTDPQKPRGDVFIPRQPSNDLFEIFEIER | | |
| | GVSADDEAKDDPGVLVHSCNFDHGLCGWIREKDNDLHWEPIRDPAGGQYLTVSAAKAPGG | | |
| | KAARLVLPLGRLMHSGDLCLSFRHKVTGLHSGTLQVFVRKHGAHGAALWGRNGGHGWRQT | | |
| | QITLRGADIKSVVFKGEKRRGHTGEIGLDDVSLKKGHCSEER | | |
| | SEQ ID NO: 115 | 691 bp | |
| NOV30f, | GGGAGGGGGCTCCGGGCGCCGCGCAGCAGACCTGCTCCCGCCGCGCGCCTCGCCGCTGTC | | |
| CG51117-02 | | | |
| DNA Sequence | CTCCGGGAGCGGCAGCAGTAGCCCGGGCGGCGAGGGCTGGCGGTTCCTCGAGACTCTCAG | | |
| | AGGGGCGCCTCCCATCGGCGCCCACCACCCCAACCTGTTCCTCGCGCGCCACTGCGCTGC | | |
| | GCCCCAGGACCCGCTGCCCAACATGGATTTTCTCCTGGCGCTGGTCCTGGTATCCTCGCT | | |
| | CTACCTGCAGGCGCCCGCCGAGTACGACGGGAGGTGGCCCAGGCAAATACTGTCATCGAT | | |
| | TGGCCTATGTCGTTATGGTGGGAGGATTGACTGCTGCTGGGGCTGGGCTCGCCAGTCTTG | | |
| | GGGACAGTGTCAGCCTTTCTACGTCTTAAGCCAGAGAATAGCCAGGATAAGGTGCCAGCT | | |
| | CAAAGCTGTGTGCCAACCACGATGCAAACATGGTGAATGTATCGGGCCAAACAAGTGCAA | | |
| | GTGTCATCCTGGTTATGCTCGAAAAACCTGTAATCAAGCCGTAGGTTTTGAAACATGTAT | | |
| | GGTTCCAGCCGGGCGCCGTGGCTCTACCCTGTAATCCCAGCACTTTGGAAGGCCGAGGCG | | |
| | GGCGGATCACGACGTCAGGATATCGAGACCATCCTGGCTAACACGGTGAAACCCCATCTC | | |
| | TACTAAAAATACAAAAAAAAAAAAAAAAAAA | | |
| | ORF Start: ATG at 203 | | ORF Stop: TAA at 572 |
| | SEQ ID NO: 116 | 123 aa | MW at 13844.1kD |
| NOV30f, | MDFLLALVLVSSLYLQAAAEYDGRWPRQIVSSIGLCRYGGRIDCCWGWARQSWGQCQPFY | | |
| CG51117-02 | | | |
| Protein Sequence | VLRQRIARIRCQLKAVCQPRCKHGECIGPNKCKCHPGYAGKTCNQAVGFERCMVPAGRRG | | |
| | STL | | |
| | SEQ ID NO: 117 | 261 bp | |
| NOV30g, | GAGTACGACGGGAGGTGGCCCACGCAAATAGTGTCATCGATTGGCCTATGTCCTTATGGT | | |
| CG51117-04 | | | |
| DNA Sequence | GGGAGGATTGACTGCTGCTGGGGCTGCCCTCGCCACTCTTGGGGACAGTGTCAGCCTGTG | | |
| | TGCCAACCACGATGCAAACATGGTCAATGTATCGGGCCAAACAAGTGCAAGTGTCATCCT | | |
| | GGTTATGCTGGAAAAACCTCTAATCAAGCCGTAGGTTTTGAAAGATGTATGCTTCCAGCC | | |
| | GGGCGCCGTGGCTCTACCCTG | | |
| | ORF Start: at 1 | | ORF Stop: end of sequence |
| | SEQ ID NO: 118 | 87 aa | MW at 9707.1kD |
| NOV30g, | EYDGRWPRQIVSSIGLCRYGGRIDCCWGWARQSWGQCQPVCQPRCKHGECIGPN | | |
| CG51117-04 | | | |
| Protein Sequence | KCKC | | |
| | HPGYAGKTCNQAVGFERCMVPAGRRGSTL | | |
| | SEQ ID NO: 119 | 1804 bp | |
| NOV30h, | CACCGGATCCATGGATTTTCTCCTGGCGCTGGTGCTGGTATCCTCGCTCTACCTGCAGGC | | |
| CG51117-08 | | | |
| DNA Sequence | GGCCGCCGAGTTCGACGGGAGGTGGCCCAGGCAAATAGTGTCATCGATTGGCCTATGTCG | | |
| | TTATGGTGGGAGGATTGACTGCTGCTGGGGCTGGGCTCGCCAGTCTTGGGGACAGTGTCA | | |
| | GCCTGTGTGCCAACCACGATGCAAACATGGTGAATGTATCGGGCCAAACAAGTGCAAGTG | | |
| | TCATCCTGGTTATGCTGGAAAAACCTGTAATCAAGACGAGCACATCCCAGCTCCTCTTGA | | |
| | CCAAGGCAGTGAACAGCCTCTTTTCCAACCCCTGGATCACCAAGCCACAAGTTTGCCTTC | | |
| | AAGGGATCTAAATGAGTGTGGCCTGAAGCCCCGGCCCTGTAAGCACAGGTGCATGAACAC | | |
| | TTACGGCAGCTACAAGTGCTACTGTCTCAACGGATATATGCTCATGCCGGATGGTTCCTG | | |
| | CTCAAGTGCCCTGACCTGCTCCATGGCAAACTGTCAGTATGGCTGTGATGTTGTTAAAGG | | |
| | ACAAATACGGTGCCAGTGCCCATCCCCTGGCCTGCACCTGGCTCCTGATGGGAGGACCTG | | |
| | TGTAGATGTTGATGAATGTGCTACAGGAAGAGCCTCCTGCCCTAGATTTAGGCAATGTGT | | |
| | CAACACTTTTGGGAGCTACATCTGCAAGTGTCATAAAGGCTTCGATCTCATGTATATTGG | | |
| | AGGCAAATATCAATGTCATCACATAGACGAATGCTCACTTGGTCAGTATCAGTGCAGCAC | | |
| | CTTTGCTCGATGTTATAACGTACGTGGGTCCTACAAGTGCAAATGTAAAGAAGGATACCA | | |
| | GGGTGATGGACTGACTTGTGTGTATATCCCAAAAGTTATGATTGAACCTTCAGGTCCAAT | | |
| | TCATGTACCAAAGGGAAATGGTACCATTTTAAAGGGTGACACAGGAAATAATAATTGGAT | | |
| | TCCTGATGTTGGAAGTACTTGGTGGCCTCCGAAGACACCATATATTCCTCCTATCATTAC | | |
| | CAACAGGCCTACTTCTAAGCCAACAACAAGACCTACACCAAAGCCAACACCAATTCCTAC | | |
| | TCCACCACCACCACCACCCCTGCCAACAGAGCTCAGAACACCTCTACCACCTACAACCCC | | |
| | AGAAAGGCCAACCACCGGACTGACAACTATAGCACCAGCTGCCAGTACACCTCCAGGACG | | |
| | GATTACAGTTGACAACAGGCTACAGACACACCCTCAGAAACCCAGAGGAGATGTGTTCAT | | |
| | TCCACGGCAACCTTCAAATGACTTGTTTGAAATATTTGAAATAGAAAGAGGAGTCAGTGC | | |
| | AGACCATGAAGCAAAGGATCATCCAGCTGTTCTGGTACACAGTTGTAATTTTGACCATGG | | |
| | ACTTTGTGGATGGATCAGGGAGAAAGACAATGACTTGCACTGGGAACCAATCAGGGACCC | | |
| | AGCAGGTGGACAATATCTCACAGTGTCGGCAGCCAAAGCCCCAGGGGGAAAAGCTGCACG | | |
| | CTTGGTGCTACCTCTCGGCCGCCTCATGCATTCAGGGGACCTGTGCCTGTCATTCAGGCA | | |
| | CAAGGTGACGCGGCTGCACTCTGGCACACTCCACGTGTTTGTGAGAAAACACGGTGCCCA | | |
| | CGGAGCAGCCCTGTGGGGAAGAAATGGTGGCCATGGCTGGAGGCAAACACAGATCACCTT | | |
| | GCGAGGGGCTGACATCAAGAGCGTCGTCTTCAAAGGTGAAAAAAGGCGTGGTCACACTGG | | |
| | GGAGATTGGATTAGATGATCTGAGCTTGAAAAAAGGCCACTGCTCTGAAGAACCCGTC+E,unsGA | | |
| | CGGC | | |
| | ORF Start: ATG at 11 | | ORF Stop: at 1796 |
| | SEQ ID NO: 120 | 595 aa | MW at 65207.8kD |
| NOV30h, | MDFLLALVLVSSLYLQAAAEFDGRWPRQIVSSIGLCRYGGRIDCCWGWARQSWGQCQPVC | | |
| CG51117-08 | | | |
| Protein Sequence | QPRCKHGECIGPNKCKCHPGYAGKTCNQDEHIPAPLDQGSEQPLFQPLDHQATSLPSRDL | | |
| | NECGLKPRPCKHRCMNTYGSYKCYCLNGYMLMPDGSCSSALTCSMANCQYGCDVVKGQIR | | |
| | CQCPSPGLHLAPDGRTCVDVDECATGRASCPRFRQCVNTFGSYICKCHKGFDLMYIGGKY | | |
| | QCHDIDECSLGQYQCSSFARCYNVRGSYKCKCKEGYQGDGLTCVYIPKVMIEPSGPIHVP | | |
| | KGNGTILKGDTGNNNWIPDVGSTWWPPKTPYIPPIITNRPTSKPTTRPTPKPTPIPTPPP | | |
| | PPPLPTELRTPLPPTTPERPTTGLTTIAPAASTPPGGITVDNRVQTDPQKPRGDVFIPRQ | | |
| | PSNDLFEIFEIERGVSADDEAKDDPGVLVHSCNFDHGLCGWIREKDNDLHWEPIRDPAGG | | |
| | QYLTVSAAKAPGGKAARLVLPLGRLMHSGDLCLSFRHKVTGLHSGTLQVFVRKHGAHGAA | | |
| | LWGRNGGHGWRQTQITLRGADIKSVVFKGEKRRGHTGEIGLDDVSLKKGHCSEER | | |
| | SEQ ID NO: 121 | 1858 bp | |
| NOV30i, | CACCGGATCCATGGATTTTCTCCTGGCGCTGCTGCTGGTATCCTCGCTCTACCTGCAGGC | | |
| CG51117-09 | | | |
| DNA Sequence | GGCCGCCGAGTTCGACCCCAGTAGGTGGCCCAGGCAAATAGTGTCATCGATTGGCCTATG | | |
| | TCGTTATGGTGGGAGGATTGACTGCTGCTGGCCCTGGGCTCGCCAGTCTTGGGGACAGTG | | |
| | TCAGCCTTTCTACGTCTTAAGGCAGAGAATACCCAGGATAAGGTGCCAGCTCAAAGCTGT | | |
| | GTGCCAACCACGATGCAAACATGGTGAATGTATCGGGCCAAACAAGTGCAAGTGTCATCC | | |
| | TGGTTATGCTGGAAAAACCTGTAATCAAGACGAGCACATCCCAGCTCCTCTTGACCAAGG | | |
| | CAGTGAACAGCCTCTTTTCCAACCCCTGGATCACCAAGCCACAAGTTTGCCTTCAAGGGA | | |
| | TCTAAATGAGTGTGGCCTGAAGCCCCGGCCCTGTAAGCACAGGTGCATGAACACTTACGG | | |
| | CAGCTACAAGTGCTACTGTCTCAACGGATATATGCTCATGCCGGATGGTTCCTGCTCAAG | | |
| | TGCCCTGACCTGCTCCATGGCAAACTGTCAGTATGGCTGTGATGTTGTTAAAGGACAAAT | | |
| | ACGGTGCCAGTCCCCATCCCCTGCCCTGCAGCTGGCTCCTCATGGGAGGACCTGTGTAGA | | |
| | TGTTGATGAATCTGCTACAGGAAGACCCTCCTGCCCTACATTTAGGCAATGTGTCAACAC | | |
| | TTTTGGGAGCTACATCTGCAAGTGTCATAAACGCTTCGATCTCATGTATATTGCACGCAA | | |
| | ATATCAATGTCATGACATAGACGAATGCTCACTTGGTCAGTATCAGTGCAGCAGCTTTGC | | |
| | TCGATGTTATAACGTACGTGGGTCCTACAACTGCAAATGTAAAGAAGGATACCAGGGTGA | | |
| | TGGACTGACTTGTGTGTATATCCCAAAAGTTATGATTGAACCTTCACGTCCAATTCATGT | | |
| | ACCAAAGGGAAATGGTACCATTTTAAAGGGTGACACAGGAAATAATAATTGGATTCCTGA | | |
| | TGTTGGAAGTACTTGGTCGCCTCCGAAGACACCATATATTCCTCCTATCATTACCAACAG | | |
| | GCCTACTTCTAACCCAACAACAAGACCTACACCAAAGCCAACACCAATTCCTACTCCACC | | |
| | ACCACCACCACCCCTGCCAACAGAGCTCAGAACACCTCTACCACCTACAACCCCAGAAAG | | |
| | GCCAACCACCGGACTGACAACTATACCACCAGCTGCCAGTACACCTCCAGGAGGGATTAC | | |
| | AGTTGACAACAGGCTACAGACAGACCCTCAGAAACCCAGAGGAGATGTGTTCATTCCACG | | |
| | GCAACCTTCAAATGACTTGTTTGAAATATTTCAAATAGAAACAGGAGTCAGTGCAGACGA | | |
| | TGAAGCAAACGATGATCCAGGTGTTCTGGTACACAGTTCTAATTTTGACCATCGACTTTG | | |
| | TGGATGGATCAGGGAGAAAGACAATGACTTGCACTGGGAACCAATCAGGGACCCAGCAGG | | |
| | TGGACAATATCTGACAGTGTCGGCAGCCAAAGCCCCAGGGGGAAAAGCTGCACGCTTGGT | | |
| | GCTACCTCTCGGCCGCCTCATGCATTCAGGGGACCTGTGCCTGTCATTCAGGCACAAGGT | | |
| | GACGGGGCTGCACTCTGGCACACTCCAGGTGTTTGTGAGAAAACACGGTGCCCACGGAGC | | |
| | AGCCCTGTGGGGAAGAAATGGTGGCCATGGCTGGAGCCAAACACAGATCACCTTGCGAGG | | |
| | GGCTGACATCAAGAGCGTCGTCTTCAAAGGTGAAAAAAGGCGTGGTCACACTGGGGAGAT | | |
| | TGGATTAGATCATCTGAGCTTGAAAAAAGGCCACTGCTCTGAACAACCCGTCGACCGC | | |
| | ORF Start: ATG at 11 | | ORF Stop: at 1850 |
| | SEQ ID NO: 122 | 613 aa | MW at 67402.4kD |

| | |
|---|---|
| NOV30i, | MDFLLALVLVSSLYLQAAAEFDGSRWPRQIVSSIGLCRYGCRIDCCWGWARQSWGQCQPF |
| CG51117-09 | |
| Protein Sequence | VYLRQRIARIRCQLKAVCQPRCKHGECIGPNKCKCHPGYAGKTCNQDEHIPAPLDQGSEQ |
| | PLFQPLDHQATSLPSRDLNECGLKPRPCKHRCMNTYCSYKCYCLNGYMLMPDGSCSSALT |
| | CSMANCQYCCDVVKGQIRCQCPSPGLQLAPDGRTCVDVDECATGRASCPRPRQCVNTFGS |
| | YICKCHKGFDLMYIGGKYQCHDIDECSLGQYQCSSFARCYNVRGSYKCKCKEGYQGDGLT |
| | CVYIPKVMIEPSGPIHVPKGNGTILKGDTGNNNWIPDVGSTWWPPKTPYIPPIITNRPTS |
| | KPTTRPTPKPTPIPTPPPPPPLPTELRTPLPPTTPERPTTGLTTIAPAASTPPGGITVDN |
| | RVQTDPQKPRGDVFIPRQPSNDLFEIFEIERGVSADDEAKDDPGVLVHSCNFDHGLCGWI |
| | REKDNDLHWEPIRDPAGGQYLTVSAAKAPGGKAARLVLPLGRLMHSGDLCLSFRHKVTGL |
| | HSGTLQVFVRKHGAHGAALWGRNGGHGWRQTQITLRGADIKSVVFKGEKRRGHTGEIGLD |
| | DVSLKKGHCSEER |

Sequence comparison of the above protein sequences yields the following sequence relationships shown in Table 30B.

TABLE 30B
Comparison of NOV30a against NOV30b through NOV30i.

| | NOV30a Residues/ | Identities/Similarities |
|---|---|---|
| Protein Sequence | Match Residues | for the Matched Region |
| NOV30b | 32 . . . 240 | 207/209 (99%) |
| | 65 . . . 273 | 207/209 (99%) |
| NOV30c | 1 . . . 240 | 210/244 (86%) |
| | 98 . . . 340 | 217/244 (88%) |
| NOV30d | 1 . . . 240 | 210/244 (86%) |
| | 81 . . . 323 | 217/244 (88%) |
| NOV30e | 32 . . . 240 | 207/209 (99%) |
| | 101 . . . 309 | 207/209 (99%) |
| NOV30f | 184 . . . 196 | 8/13 (61%) |
| | 88 . . . 100 | 8/13 (61%) |
| NOV30g | 167 . . . 196 | 14/32 (43%) |
| | 33 . . . 64 | 15/32 (46%) |
| NOV30h | 1 . . . 240 | 210/244 (86%) |
| | 80 . . . 322 | 216/244 (88%) |
| NOV30i | 1 . . . 240 | 211/244 (86%) |
| | 98 . . . 340 | 217/244 (88%) |

Further analysis of the NOV30a protein yielded the following properties shown in Table 30C.

TABLE 30C
Protein Sequence Properties NOV30a

| | |
|---|---|
| PSort analysis: | 0.5500 probability located in endoplasmic |
| | reticulum (membrane); 0.1900 probability located in |
| | lysosome (lumen); 0.1000 probability located in |
| | endoplasmic reticulum (lumen); 0.1000 probability |
| | located in outside |
| SignalP analysis: | No Known Signal Sequence Predicted |

A search of the NOV30a protein against the Geneseq database, a proprietary database that contains sequences published in patents and patent publication, yielded several homologous proteins shown in Table 30D.

TABLE 30D
Geneseq Results for NOV30a

| | | NOV30a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB70549 | Clone 16467945.0.85-S261.D | 32 . . . 450 | 417/419 (99%) | 0.0 |
| | protein sequence SEQ ID | 65 . . . 483 | 417/419 (99%) | |
| | NO: 82 - Homo sapiens, 546 | | | |
| | aa. [WO200110902-A2, 15- | | | |
| | FEB-2001] | | | |
| AAB70547 | Human PRO17 protein | 32 . . . 450 | 417/419 (99%) | 0.0 |
| | sequence SEQ ID NO: 34 - | 101 . . . 519 | 417/419 (99%) | |
| | Homo sapiens, 582 aa. | | | |
| | [WO200110902-A2, 15- | | | |
| | FEB-2001] | | | |
| AAB80265 | Human PRO334 protein - | 36 . . . 450 | 383/415 (92%) | 0.0 |
| | Homo sapiens, 509 aa. | 88 . . . 473 | 383/415 (92%) | |
| | [WO200104311-A1, 18- | | | |
| | JAN-2001] | | | |
| AAU29049 | Human PRO polypeptide | 36 . . . 450 | 383/415 (92%) | 0.0 |
| | sequence #26 - Homo | 88 . . . 473 | 383/415 (92%) | |
| | sapiens, 509 aa. | | | |
| | [WO200168848-A2, 20-SEP- | | | |
| | 2001] | | | |
| AAY13397 | Amino acid sequence of | 36 . . . 450 | 383/415 (92%) | 0.0 |
| | protein PRO334 - Homo | 88 . . . 473 | 383/415 (92%) | |
| | sapiens, 509 aa. | | | |
| | [WO9914328-A2, 25-MAR- | | | |
| | 1999] | | | |

In a BLAST search of public sequence datbases, the NOV30a protein was found to have homology to the proteins shown in the BLASTP data in Table 30E.

TABLE 30E
Public BLASTP Results for NOV30a

| | | NOV30a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC33425 | Sequence 33 from Patent | 32 . . . 450 | 417/419 (99%) | 0.0 |
| | WO0110902 - Homo sapiens | 101 . . . 519 | 417/419 (99%) | |
| | (Human), 582 aa. | | | |
| Q91V88 | POEM (NEPHRONECTIN | 36 . . . 450 | 363/416 (87%) | 0.0 |
| | short isoform) - Mus | 88 . . . 502 | 386/416 (92%) | |
| | musculus (Mouse), 561 aa. | | | |
| Q91ZD3 | Nephronectin long isoform - | 36 . . . 450 | 363/416 (87%) | 0.0 |
| | Mus musculus (Mouse), 578 | 105 . . . 519 | 386/416 (92%) | |
| | aa. | | | |
| Q91XL5 | Nephronectin - Mus musculus | 38 . . . 450 | 362/414 (87%) | 0.0 |
| | (Mouse), 592 aa. | 121 . . . 533 | 385/414 (92%) | |
| Q923T5 | Nephronectin - Mus musculus | 38 . . . 450 | 362/414 (87%) | 0.0 |
| | (Mouse), 609 aa. | 138 . . . 550 | 385/414 (92%) | |

PFam analysis predicts that the NOV30a protein contains the domains shown in Table 30F.

TABLE 30F
Domain Analysis of NOV30a

| | | Identities/ | |
|---|---|---|---|
| | | Similarities | |
| | NOV30a | for the Matched | |
| Pfam Domain | Match Region | Region | Expect Value |
| EGF | 41 . . . 75 | 15/47 (32%) | 0.84 |
| | | 27/47 (57%) | |
| EGF | 81 . . . 115 | 10/47 (21%) | 0.034 |
| | | 24/47 (51%) | |
| EGF | 166 . . . 201 | 12/47 (26%) | 4.9e−06 |
| | | 29/47 (62%) | |

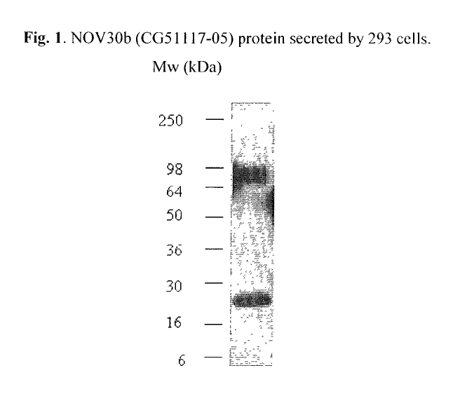

FIG. 1 shows that NOV30b (G51117-05) is expressed as about 66 kDa protein secreted by 293 cells.

### Example 31

The NOV31 clone was analyzed, and the nucleotide and encoded polypeptide sequences are shown in Table 31A.

TABLE 31A
NOV31 Sequence Analysis

| | SEQ ID NO: 123 | 3336 bp | |
|---|---|---|---|
| NOV31a, | CGCCGGTGGCTCGGCGGCGGCCGCGGCGGCGGCGGCGGCGGCGGCCGCCGCGTCGTCTAC | | |
| CG51264-01 | | | |
| DNA Sequence | CTCCAGCTCCTCCTCCCTCCTCCTCCGTCTCCTCCTCTCTCTCTCCATCTGCTGTGGTTA | | |
| | TGGCCTGTCGCTGGAGCACAAAAGACTCTCCGCGGTGGACGTCTGCGTTGCTCTTGCTTT | | |
| | TCCTCGCTGGGGTGTACGCTTGTGGAGAGACTCCACAGCAAATACGAGCACCAAGTGGCA | | |
| | TAATCACAAGCCCAGGCTGGCCTTCTGAATATCCTGCAAAAATCAACTGTAGCTGGTTCA | | |
| | TAAGGGCAAACCCAGCCGAAATCATTACTATAAGTTTTCAGCATTTTGATATTCAAGGAT | | |
| | CCAGAAGGTGCAATTTGGACTGGTTGACAATAGAAACATACAAGAATATTGAAAGTTACA | | |
| | GAGCTTCTGGTTCCACAATTCCACCTCCGTATATCTCTTCACAAGACCACATCTCGATTA | | |
| | GGTTTCATTCGGATGACAACATCTCTAGAAAGGGTTTCAGACTGGCATATTTTTCAGGGA | | |
| | AATCTGAGGAACCAAATTGTGCTTGTGATCAGTTTCGTTGTGGTAATGGAAAGTGTATAC | | |
| | CAGAAGCCTCGAAATCCAATAACATGGATGAATGTGGAGATACTTCCGATGAACAGATCT | | |
| | GTGCCAAACAACCAAATCCTCCAACTCCTCCTGCTTTTCAACCCTGTGCTTACAACCAGT | | |
| | TCCAGTCTTTATCCCGTTTTACCAAAGTTTACACTTGCCTCCCCGAATCTTTAAAATGTC | | |
| | ATGGCAACATTGACTGCCTTGACCTACGAGATGAGATAGACTGTCATGTGCCAACATGTG | | |
| | GGCAATGGCTAAAATATTTTTATGGTACTTTTAATTCTCCCAATTATCCAGACTTTTATC | | |
| | CTCCTCGAAGCAATTGCACCTGGTTAATAGACACTGGTGATCACCGTAAACTCATTTTAC | | |
| | GCTTCACTCACTTTAAACTTGATGGTACTGGTTATGCTGATTATGTCAAAATATATCATC | | |
| | GATTACAGCAGAATCCACACAACCTTTTGCGTGTGTTGACAGCTTTTGATTCTCATGCAC | | |
| | CTCTTACAGTTGTTTCTTCTTCTGGACAGATAAGGGTACATTTTTCTGCTGATAAAGTGA | | |
| | ATGCTGCAAGGCGATTTAATCCTACTTACCAAGTAGATGGGTTCTGTTTGCCATCGGAAA | | |
| | TACCCTGTGCACGTAACTGGGGGTGTTATACTGAGCAGCAGCCTTGTGATGGCTATTGGC | | |
| | ATTGCCCAAATGGAAGGGATGAAACCAATTGTACCATGTGCCAGAAGGAACAATTTCCAT | | |
| | GTTCCCGAAATGGTGTCTCTTATCCTCGTTCTGATCGCTGCAACTACCACAATCATTGCC | | |
| | CAAATGCCTCAGATGAAAAAAACTCCTTTTTTTCCCAACCAGGAAATTTCCATTGTAAAA | | |
| | ACAATCGTTGTGTGTTTGAAAGTTGGGTGTGTGATTCTCAAGATGACTCTGGTGATGGCA | | |
| | GCGATGAAGAAAATTGCCCAGTAATCGTGCCTACAACAGTCATCACTGCTGCCGTCATAG | | |
| | GGAGCCTCATCTGTGCCCTGTTACTCGTCATAGCATTGGGATGTACTTGTAAGCTTTATT | | |
| | CTCTGAGAATGTTTGAAAGAACATCATTTCAAACACAGTTGTCAAGAGTGGAAGCAGAAT | | |
| | TGTTAAGAAGACAAGCTCCTCCCTCGTATCCACAATTGATTGCTCAGGGTTTAATTCCAC | | |
| | CAGTTGAAGATTTTCCTGTTTCTTCACCTAATCAGGCTTCTGTTTTGGAAAATCTGAGGC | | |
| | TAGCGGTACGATCTCAGCTTCGATTTACTTCAGTCACGCTTCCTATGGCAGCCAGATCAA | | |
| | GCAACATTTCGAACCGTATTTTTAATTTTGCAAGATCACGTCATTCTGGGTCATTCGCTT | | |
| | TGGTCTCACCAGATGGACATGAGGTTGTCCCTAGTCAGAGTACCAGTAGAGAACCTGAGA | | |
| | GAAATCATACTCACACAAGTTTGTTTTCCGTGCAGTCTGATGATACAGACACAGAAAATG | | |
| | AGAGAACAGATATGGCAGGAGCATCTGGTGGGCTTGCAGCTCCTTTGCCTCAAAAAGTCC | | |
| | CTCCCACAACGGCAGTAGAAGCGACAGTAGGAGCATCTGCAAGTTCCTCAACTCAGAGTA | | |
| | CCCCAGGTGGTCATGCAGATAATGCAAGGGATGTGACAACTGTGGAACCCCCAACTGTGA | | |
| | GTCCAGCACCTCACCACCTTACAAGTGCACTCAGTCGTATGACTCAGGGGCTACGCTGGG | | |
| | TACGTTTTACATTACCACGATCAAGTTCCCTAAGTCAGAACCACAGTCCTTTGAGACAAC | | |
| | TTGATAATGGGGTAAGTGGAAGAGAAGATGATGATGATGTTGAAATGCTAATTCCAATTT | | |
| | CTGATGGATCTTCAGACTTTGATGTGAATGACTGCTCCAGACCTCTTCTTGATCTTGCCT | | |
| | CAGATCAAGGACAAGGGCTTAGACAACCATATAATGCAACAAATCCTGGAGTAAGGCCAA | | |
| | GTAATCGAGATGGCCCCTGTGAGCGCTGTGGTATTGTCCACACTCCCCAGATACCAGACA | | |
| | CTTGCTTAGAAGTAACACTGAAAAACGAAACGAGTCATGATGAGGCTTTCTTACTTTGTT | | |
| | AGGTACGAATCACATAAGGGAGATTGTATACAAGTTGGAGCAATATCCATTTATTATTTT | | |
| | GTAACTTTACAGTTAAACTAGTTTTAGTTTAAAAAGAAAAAATGCAGGGTGATTTCTTAT | | |
| | TATTATATGTTAGCCTGCATGGTTAAATTCGACAACTTGTAACTCTATGAACTTAGAGTT | | |
| | ATTTGTTTCTGATTGTTTTCATCCTGATACTGTAGTTCACTGTAGAAATGTGGCTGCTGA | | |
| | AACTCATTTGATTGTCATTTTTATCTATCCTATGTTAAATGGTTTGTTTTTACAAAATAA | | |
| | TACCTTATTTTAATTGAAACGTTTATGCTTTTGCCAAGCACATCTTGTAACTTAATATAG | | |
| | ATTTGTTTCTGATTGTTTTCATCCTGATACTGTAGTTCACTGTAGAAATGTGGCTGCTGA | | |
| | AACTCATTTGATTGTCATTTTTATCTATCCTATGTTAAATGGTTTGTTTTTACAAAATAA | | |
| | TACCTTATTTTAATTGAAACGTTTATGCTTTTGCCAAGCACATCTTGTAACTTAATATAG | | |
| | CTAGATGTTAAGGTTGTTAATGTACCAAAAAAAAAAAACCTTATACTCACCTGCGTTTTC | | |
| | ATTTGTTTGACATTTGTCTATTATTGGATATCATTATCATATGAACTTGTCAGTGGGAAA | | |
| | CAAACTGTCTAAAAATTTATCTCTTACGTTTAACATACAATCATGTGAGATTTAGGCAGA | | |
| | GTTCGATAAATTACTGGCAAAAACAAAACTCATTTATAAAGATTTTCTAATGTTGACTTT | | |
| | AATACTCTAACATGGTACAAACCANATGGTAAAATC | | |
| | ORF Start: ATG at 120 | | ORF Stop: TAG at 2640 |
| | SEQ ID NO: 124 | 840 aa | MW at 93121.8kD |
| NOV31a, | MACRWSTKESPRWRSALLLLFLAGVYACGETPEQIRAPSGIITSPGWPSEYPAKINCSWF | | |
| CG51264-01 | | | |
| Protein Sequence | IRANPGEIITISFQDFDIQGSRRCNLDWLTIETYKNIESYRACGSTIPPPYISSQDHIWI | | |
| | RFHSDDNISRKGFRLAYFSGKSEEPNCACDQFRCGNGKCIPEAWKCNNMDECGDSSDEEI | | |
| | CAKEANPPTAAAFQPCAYNQFQCLSRFTKVYTCLPESLKCDGNIDCLDLGDEIDCDVPTC | | |
| | GQWLKYFYGTFNSPNYPDFYPPGSNCTWLIDTGDHRKVILRFTDFKLDGTGYGDYVKIYD | | |
| | GLEENPHKLLRVLTAFDSHAPLTVVSSSGQIRVHFCADKVNAARGFNATYQVDGFCLPWE | | |
| | IPCGGNWGCYTEQQRCDGYWHCPNGRDETNCTMCQKEEFPCSRNGVCYPRSDRCNYQNHC | | |
| | PNGSDEKNCFFCQPGNFHCKNNRCVFESWVCDSQDDCGDGSDEENCPVIVPTRVITAAVI | | |
| | GSLICGLLLVIALGCTCKLYSLRMFERRSFETQLSRVEAELLRREAPPSYGQLIAQGLIP | | |
| | PVEDFPVCSPNQASVLENLRLAVRSQLGFTSVRLPMAGRSSNIWNRIFNFARSRHSGSLA | | |
| | LVSADGDEVVPSQSTSREPERNHTHRSLFSVESDDTDTENERRDMAGASGGVAAPLPQKV | | |
| | PPTTAVEATVGACASSSTQSTRGGHADNGRDVTSVEPPSVSPARHQLTSALSRMTQGLRW | | |
| | VRFTLGRSSSLSQNQSPLRQLDNGVSGREDDDDVEMLIPISDGSSDFDVNDCSRPLLDLA | | |
| | SDQGQGLRQPYNATNPGVRPSNRDGPCERCGIVHTAQIPDTCLEVTLKNETSDDEALLLC | | |
| | SEQ ID NO: 125 | 498 bp | |
| NOV31b | CGCCGGTGGCTCGGCGGCGGCGGCGGCGGCGGCGGCGGCGGCGGCGGCGGCGTCGTCTAC | | |
| CG51264-03 | | | |
| DNA Sequence | CTCCAGCTCCTCCTCCCTCCTCCTCCCTCTCCTCCTCTCTCTCTCCATCTGCTGTGGTTA | | |
| | TGGCCTGTCGCTCGAGCACAAAAGAGTCTCCGCGGTGGAGGTCTGCGTTGCTCTTGCTTT | | |
| | TCCTCGCTGGGGTGTACGGAAATGGTGCTCTTGCAGAACATTCTGAAAATGTGCATATTT | | |
| | CAGGAGTGTCAACTGCTTGTGGAGAGACTCCAGAGCAAATACGAGCACCAAGTGGCATAA | | |
| | TCACAAGCCCAGGCTGGCCTTCTGAATATCCTGCAAAAATCAACTGTAGCTGGTTCATAA | | |
| | GGGCAAACCCAGGCGAAATCATTACTATAAGTTTTCAGGATTTTGATATTCAAGGATCCA | | |
| | GAAGGTGCAATTTGGACTGGTTGACAATAGAAACATACAAGAATATTGAAAGTTACAGAG | | |
| | CTTGTGGTTCCACAATTCCACCTCCGTATATCTCTTCACAAGACCACATCTGGATTAGGT | | |
| | TTCATTCGGATGACAACATCTCTAGAAAGGGTTTCAGACTGGCATATTTTTCAGGGAAAT | | |
| | CTGAGGAACCAAATTGTGCTTGTGATCAGTTTCGTTGTGGTAATGGAAAGTGTATACCAG | | |
| | AAGCCTGGAAATGTAATAACATGGATGAATGTGGAGATAGTTCCGATGAAGAGATCTGTG | | |
| | CCAAAGAAGCAAATCCTCCAACTGCTGCTGCTTTTCAACCCTGTGCTTACAACCAGTTCC | | |
| | AGTGTTTATCCCGTTTTACCAAAGTTTACACTTGCCTCCCCGAATCTTTAAAATGTGATG | | |
| | GGAACATTGACTGCCTTGACCTAGGAGATGAGATAGACTGTGATGTGCCAACATGTGGGC | | |
| | AATGGCTAAAATATTTTTATGGTACTTTTAATTCTCCCAATTATCCAGACTTTTATCCTC | | |
| | CTGGAAGCAATTGCACCTGGTTAATAGACACTGGTGATCACCGTAAAGTCATTTTACGCT | | |
| | TCACTGACTTTAAACTTGATGGTACTGGTTATGGTGATTATGTCAAAATATATGATGGAT | | |
| | TAGAGGAGAATCCACACAAGCTTTTGCGTGTGTTGACAGCTTTTGATTCTCATGCACCTC | | |
| | TTACAGTTGTTTCTTCTTCTGGACAGATAAGGGTACATTTTTGTGCTGATAAAGTGAATG | | |
| | CTGCAAGGGGATTTAATGCTACTTACCAAGTAGATGGGTTCTGTTTGCCATGGGAAATAC | | |
| | CCTGTGGAGGTAACTGGGGGTGTTATACTGAGCAGCAGCGTCGTGATGGGTATTGGCATT | | |
| | GCCCAAATGGAAGGGATGAAACCAATTGTACCATGTGCCAGAAGGAAGAATTTCCATGTT | | |
| | CCCGAAATGGTGTCTGTTATCCTCGTTCTGATCGCTGCAACTACCACAATCATTGCCCAA | | |
| | ATGCCAAACAGAACCCATCTACTTGCTAAGTACCATTAAATCCCCTTGCAGCATTCAC | | |
| | ORF Start: ATG at 120 | | ORF Stop: TAA at 1467 |
| | SEQ ID NO: 126 | 449 aa | MW at 50654.0kD |
| NOV31b, | MACRWSTKESPRWRSALLLLFLAGVYGNCALAEHSENVHISGVSTACGETPEQIRAPSGI | | |
| CG51264-03 | | | |
| Protein Sequence | ITSPGWPSEYPAKTNCSWPIRANPGEIITISFQDFDIQOSRRCNLDWLTIETYKNIESYR | | |
| | ACGSTIPPPYISSQDHIWIRFHSDDNISRKGFRLAYFSGKSEEPNCACDQFRCGNGKCIP | | |
| | EAWKCNNMDECGDSSDEEICAKEANPPTAAAFQPCAYNQFQCLSRFTKVYTCLPESLKCD | | |
| | GNIDCLDLGDEIDCDVPTCGQWLKYFYGTFNSPNYPDFYPPGSNCTWLIDTGDHRKVILR | | |
| | FTDFKLDGTGYGDYVKIYDCLEENPHKLLRVLTAFDSHAPLTVVSSSGQIRVHFCADKVN | | |
| | AARGFNATYQVDGFCLPWEIPCGGNWGCYTEQQRRDGYWHCPNGRDETNCTMCQKEEFPC | | |
| | SRNGVCYPRSDRCNYQNHCPNGKQNPSTW | | |
| | SEQ ID NO: 127 | 1441 bp | |
| NOV31c, | CGCCGGTGGCTCGGCGGCGGCCGCGGCGGCGGCGGCGGCGGCGGCGGCGGCGTCCTCTAC | | |
| CG51264-04 | | | |
| DNA Sequence | CTCCAGCTTCTCCTCCCTCCTCCTCCGTCTCCTCCTCTCTCTCTCCATCTGCTGTGGTTA | | |
| | TGGCCTGTCGCTGGAGCACAAAAGAGTCTCCGCGGTGGAGGTCTCCGTTGCTCTTGCTTT | | |
| | TCCTCCCTGGGGTGTACGCTTGTGGAGAGACTCCAGAGCAAATACGAGCACCAAGTGGCA | | |
| | TAATCACAAGCCCAGGCTGGCCTTCTGAATATCCTGCAAAAATCAACTGTAGCTGGTTCA | | |
| | TAAGGGCAAACCCAGGCGAAATCATTACTATAAGTTTTCAGGATTTTGATATTCAAGGAT | | |
| | CCAGAAGGTGCAATTTGGACTGGTTGACAATAGAAACATACAAGAATATTGAAAGTTACA | | |
| | GAGCTTGTGGTTCCACAATTCCACCTCCGTATATCTCTTCACAAGACCACATCTGGATTA | | |
| | GGTTTCATTCGGATGACAACATCTCTAGAAAGGGTTTCAGACTGGCATATTTTTCAGGGA | | |
| | AATCTGAGGAACCAAATTGTCCTTGTGATCAGTTTCGTTGTGCTAATGGAAAGTCTATAC | | |
| | CAGAAGCCTGGAAATGTAATAACATGCATGAATGTGGAGATAGTTCCGATGAAGAGATCT | | |
| | GTGCCAAAGAAGCAAATCCTCCAACTGCTGCTGCTTTTCAACCCTGTGCTTACAACCAGT | | |
| | TCCAGTGTTTATCCCGTTTTACCAAAGTTTACACTTCCCTCCCCGAATCTTTAAAATGTG | | |
| | ATGGGAACATTGACTGCCTTCACCTAGGACATGAGATAGACTGTGATGTGCCAACATGTG | | |
| | GGCAATGGCTAAAATATTTTTATGGTACTTTTAATTCTCCCAATTATCCAGACTTTTATC | | |
| | CTCCTGGAACCAATTGCACCTGGTTAATAGACACTGGTGATCACCGTAAAGTCATTTTAC | | |
| | GCTTCACTGACTTTAAACTTGATCGTACTGGTTATGGTGATTATGTCAAAATATATGATG | | |
| | GATTAGAGGAGAATCCACACAAGCTTTTGCGTGTGTTGACAGCTTTTGATTCTCATGCAC | | |
| | CTCTTACAGTTGTTTCTTCTTCTCGACAGATAAGGGTACATTTTTGTGCTGATPAAGTGA | | |
| | ATGCTGCAAGGGGATTTAATGCTACTTACCAAGTAGATCGGTTCTGTTTGCCATGGGAAA | | |
| | TACCCTGTGGAGGTAACTGGGGCTGTTATACTGAGCAGCAGCGTCGTGATGGGTATTCGC | | |
| | ATTGCCCAAATGGAAGGGATGAAACCAATTGTACCATGTGCCAGAAGGAAGAATTTCCAT | | |
| | GTTCCCGAAATGGTGTCTGCTATCCTCGCTCTGATCGCTGCAACTACCAGAATCATTGCC | | |
| | CAAATGGCAAACAGAACCCATCTACTTGGTAAGTAGCATTAAATCCCCTTGCAGCATTCA | | |
| | C | | |
| | ORF Start: ATG at 120 | | ORF Stop: TAA at 1410 |
| | SEQ ID NO: 128 | 430 aa | MW at 48793.0kD |
| NOV31c, | MACRWSTKESPRWRSALLLLFLAGVYACCETPEQIRAPSGIITSPGWPSEYPAKINCSWF | | |
| CG51264-04 | | | |
| Protein Sequence | IRANPGEIITISFQDFDIQGSRRCNLDWLTIETYKNIESYRACGSTIPPPYISSQDHIWI | | |
| | RFHSDDNISRKGFRLAYFSCKSEEPNCACDQFRCGNGKCIPEAWKCNNNDECGDSSDEEI | | |
| | CAKEANPPTAAAFQPCAYNQFQCLSRFTKVYTCLPESLKCDGNIDCLDLGDEIDCDVPTC | | |
| | GQWLKYFYGTFNSPNYPDFYPPGSNCTWLIDTGDHRKVILRFTDFKLDGTCYGDYVKIYD | | |
| | GLEENPHKLLRVLTAFDSHAPLTVVSSSGQIRVHFCADKVNAARGFNATYQVDGFCLPWE | | |
| | IPCGGNWGCYTEQQRRDGYWHCPNGRDETNCTMCQKEEFPCSRNGVCYPRSDRCNYQNHC | | |
| | PNGKQNPSTW | | |
| | SEQ ID NO: 129 | 3021 bp | |
| NOV31d, | CTCCTCCTCCGTCTCCTCCTCTCTCTCTCATCTGCTGTGGTTATGGCCTGTCGCTGGAGC | | |
| CG51264-06 | | | |
| DNA Sequence | ACAAAAGAGTCTCCGCGGTGGAGGTCTGCGTTGCTCTTGCTTTTCCTCGCTGGGGTGTAC | | |
| | GCTTGTGGAGACACTCCAGAGCAAATACGAGCACCAAGTGGCATAATCACAAGCCCAGGC | | |
| | TGGCCTTCTGAATATCCTCCAAAAATCAACTGTAGCTGGTTCATAAGGGCAAACCCAGCC | | |
| | GAAATCATTACTATAAGTTTTCAGGATTTTGATATTCAAGGATCCAGAAGGTGCAATTTG | | |
| | GACTGGTTGACAATAGAAACATACAAGAATATTGAAAGTTACAGAGCTTGTGGTTCCACA | | |
| | ATTCCACCTCCGTATATCTCTTCACAAGACCACATCTGGATTAGGTTTCATTCGGATGAC | | |
| | AACATCTCTAGAAACGGTTTCACACTCGCATATTTTTCAGGGAAATCTGAGGAACCAAAT | | |
| | TGTGCTTGTGATCAGTTTCGTTGTGGTAATGGAAAGTGTATACCACAAGCCTGGAAATGT | | |
| | AATAACATGGATGAATGTGGAGATAGTTCCGATGAAGAGATCTGTGCCAAAGAAGCAAAT | | |
| | CCTCCAACTGCTGCTGCTTTTCAACCCTGTGCTTACAACCAGTTCCAGTGTTTATCCCGT | | |
| | TTTACCAAAGTTTACACTTGCCTCCCCGAATCTTTAAAATGTGATCGCAACATTGACTCC | | |
| | CTTGACCTAGGAGATGAGATAGACTGTGATGTCCCAACATGTGGGCAATGGCTAAAATAT | | |
| | TTTTATGGTACTTTTAATTCTCCCAATTATCCAGACTTTTATCCTCCTGGAAGCAATTGC | | |
| | ACCTGGTTAATAGACACTCCTGATCACCGTAAAGTCATTTTACGCTTCACTGACTTTAAA | | |
| | CTTGATGGTACTGGTTATGGTGATTATGTCAAAATATATGATGGATTAGAGGAGAATCCA | | |
| | CACAAGCTTTTGCGTGTGTTGACACCTTTTGATTCTCATGCACCTCTTACAGTTGTTTCT | | |
| | TCTTCTGGACAGATAAGGGTACATTTTTGTGCTGATAAAGTGAATCCTGCAAGCGCATTT | | |
| | AATGCTACTTACCAAGTAGATGGGTTCTGTTTGCCATGGGAAATACCCTGTCGAGGTAAC | | |
| | TGGGCGTCTTATACTGAGCAGCAGCGTTCTGATGGGTATTCGCATTGCCCAAATGGAACG | | |
| | GATGAAACCAATTGTACCATGTGCCAGAAGCAACAATTTCCATGTTCCCGAAATGGTGTC | | |
| | TGTTATCCTCGTTCTGATCGCTGCAACTACCAGAATCATTCCCCAAATGCCTCAGATGAA | | |
| | AAAAACTGCTTTTTTTGCCAACCAGGAAATTTCCATTGTTAAAACAATCGTTGTGTGTTT | | |
| | GAAAGTTGGGTGTGTGATTCTCAAGATGACTGTGGTGATGGCAGCGATGAAGAAAATTGC | | |
| | CCAGTAATCGTGCCTACAAGAGTCATCACTGCTGCCGTCATAGCGAGCCTCATCTGTGGC | | |
| | CTGTTACTCGTCATAGCATTGGGATGTACTTGTAAGCTTTATTCTCTGAGAATGTTTGAA | | |
| | AGAAGATCATTTGAAACACAGTTGTCAAGAGTGGAAGCACAATTGTTAAGAAGAGAAGCT | | |
| | CCTCCCTCGTATGGACAATTGATTGCTCAGGGTTTAATTCCACCAGTTGAAGATTTTCCT | | |
| | GTTTGTTCACCTAATCAGGCTTCTCTTTTGGAAAATCTGAGGCTAGCGGTACGATCTCAG | | |
| | CTTGGATTTACTTCAGTCAGGCTTCCTATGGCAGGCAGATCAAGCAACATTTGGAACCGT | | |
| | ATTTTTAATTTTGCAAGATCACGTCATTCTGGGTCATTGGCTTTCCTCTCAGCAGATGGA | | |
| | GATGAGGTTCTCCCTAGTCAGAGTACCAGTAGAGAACCTGAGAGAAATCATACTCACAGA | | |
| | AGTTTGTTTTCCGTGCAGTCTGATGATACACACACAGAAAATCAGAGAAGAGATATGGCA | | |
| | GGAGCATCTGGTGGGGTTGCAGCTCCTTTGCCTCAAAAAGTCCCTCCCACAACGGCAGTG | | |
| | GAAGCGACAGTAGGAGCATGTCCAAGTTCCTCAACTCAGAGTACCCGAGGTGGTCATGCA | | |
| | GATAATGGAACGCATGTGACAAGTGTGGAACCCCCAAGTGTGAGTCCAGCACGTCACCAG | | |
| | CTTACAAGTGCACTCAGTCGTATGACTCAGGGGCTACGCTGGGTACGTTTTACATTAGGA | | |
| | CGATCAAGTTCCCTAAGTCAGAACCAGAGTCCTTTGACACAACTTGATAATGGGGTAAGT | | |
| | GGAAGAGAAGATGATGATGATCTTCAAATGCTAATTCCAATTTCTGATGGATCTTCAGAC | | |
| | TTTGATGTGAATGACTGCTCCAGACCTCTTCTTGATCTTGCCTCACATCAAGGACAAGGC | | |
| | CTTAGACAACCATATAATGCAACAAATCCTGGAGTAAGGCCAAGTAATCGAGATGGCCCC | | |
| | TGTGAGCGCTGTCGTATTGTCCACACTGCCCAGATACCAGACACTTGCTTAGAAGTAACA | | |
| | CTGAAAAACGAAACCAGTGATGATGAGGCTTTGTTACTTTGTTAGGTACGAATCACATAA | | |
| | GGGAGATTCTATACAAGTTGGAGCAATATCCATTTATTATTTTGTAACTTTACAGTTAAA | | |
| | CTAGTTTTAGTTTAAAAAGAAAAAATGCAGGGTGATTTCTTATTATTATATGTTAGCCTG | | |
| | AAAATGCATCACATATTGCATATTGTTCAATAATGGTCCTTTCATTTGTTTCTGATTGTT | | |
| | TTCATCCTGATACTGTAGTTCACTGTAGAAATGTGGCTGCTGAAACTCATTTGATTGTCA | | |
| | TTTTTATCTATCCTATGTTAAATGGTTTGTTTTTACAAAATAATACCTTATTTTAATTGA | | |
| | AACGTTTATGCTTTTGCCAAGCACATCTTGTAACTTAATATAGCTAGATGTTAAGGTTGT | | |
| | TAATGTACCAAAAAAAAAAAA | | |
| | ORF Start: ATG at 43 | | ORF Stop: TAG at 2563 |
| | SEQ ID NO: 130 | 840 aa | MW at 93121.8kD |
| NOV31d, | MACRWSTKESPRWRSALLLLFLAGVYACGETPEQIRAPSGIITSPGWPSEYPAKINCSWF | | |
| CG51264-06 | | | |
| Protein Sequence | IRANPGEIITISFQDFDIQGSRRCNLDWLTIETYKNIESYRACGSTIPPPYISSQDHIWI | | |
| | RFHSDDNISRKGFRLAYFSGKSEEPNCACDQFRCGNGKCIPEAWKCNNMDECGDSSDEEI | | |
| | CAKEANPPTAAAFQPCAYNQFQCLSRFTKVYTCLPESLKCDGNIDCLDLGDEIDCDVPTC | | |
| | GQWLKYFYGTFNSPNYPDFYPPGSNCTWLIDTGDHRKVILRFTDFKLDGTGYGDYVKIYD | | |
| | GLEENPHKLLRVLTAFDSHAPLTVVSSSGQIRVHFCADKVNAARGFNATYQVDGFCLPWE | | |
| | IPCGGNWGCYTEQQRCDGYWHCPNGRDETNCTMCQKEEFPCSRNGVCYPRSDRCNYQNHC | | |
| | PNGSDEKNCFFCQPGNFHCKNNRCVFESWVCDSQDDCGDGSDEENCPVIVPTRVITAAVI | | |
| | GSLICGLLLVIALGCTCKLYSLRMFERRSFETQLSRVEAELLRREAPPSYGQLIAQGLIP | | |
| | LVSADGDEVVPSQSTSREPERNHTHRSLFSVESDDTDTENERRDMAGASGGVAAPLPQKV | | |
| | PPTTAVEATVGACASSSTQSTRGGHADNGRDVTSVEPPSVSPARHQLTSALSRMTQGLRW | | |
| | VRFTLGRSSSLSQNQSPLRQLDNGVSGREDDDDVEMLIPISDGSSDFDVNDCSRPLLDLA | | |
| | SDQGQGLRQPYNATNPGVRPSNRDGPCERCGIVHTAQIPDTCLEVTLKNETSDDEALLLC | | |
| | SEQ ID NO: 131 | 3012 bp | |
| NOV31e, | CTCCTCCTCCGTCTCCTCCTCTCTCTCTCATCTGCTCTCGTTATGGCCTGTCGCTGGAGC | | |
| CG51264-07 | | | |
| DNA Sequence | ACAAAAGACTCTCCGCGGTGGAGGTCTGCGTTGCTCTTGCTTTTCCTCGCTGGGGTGTAC | | |
| | GCTGTGAGAACTCAACAATACAGCACAAGTGGCATAATCACAAGCCCAGGCTGGCCTTCT | | |
| | GAATATCCTCCAAAAATCAACTGTAGCTGGTTCATAAGGCCAAACCCAGGCGAAATCATT | | |
| | ACTATAAGTTTTCAGGATTTTCATATTCAAGGATCCAGAAGGTGCAATTTGGACTGGTTC | | |
| | ACAATAGAAACATACAAGAATATTGAAAGTTACAGAGCTTGTGGTTCCACAATTCCACCT | | |
| | CCGTATATCTCTTCACAAGACCACATCTGGATTAGGTTTCATTCGGATGACAACATCTCT | | |
| | AGAAAGCCTTTCAGACTCGCATATCTTTCAGGCAAATCTGAGCAACCAAATTCTGCTTCT | | |
| | GATCAGTTTCGTTCTGGTAATGGAAAGTGTATACCACAAGCCTGGAAATCTAATAACATG | | |
| | GATGAATGTGGAGATAGTTCCGATCAAGAGATCTGTGCCAAAGAAGCAAATCCTCCAACT | | |
| | GCTGCTGCTTTTCAACCCTGTGCTTACAACCAGTTCCAGTGTTTATCCCGTTTTACCAAA | | |
| | GTTTACACTTCCCTCCCCGAATCTTTAAAATGTGATGGGAACATTCACTGCCTTCACCTA | | |
| | GGAGATGAGATAGACTGTGATGTGCCAACATGTGGGCAATGGCTAAAATATTTTTATGGT | | |
| | ACTTTTAATTCTCCCAATTATCCAGACTTTTATCCTCCTGGAAGCAATTGCACCTGGTTA | | |
| | ATACACACTGGTGATCACCGTAAAGTCATTTTACGCTTCACTGACTTTAAACTTGATGGT | | |
| | ACTGGTTATGGTGATTATGTCAAAATATATGATGGATTAGAGAAGAATCCACACAAGCTT | | |
| | TTGCGTGTGTTGACAGCTTTTGATTCTCATGCACCTCTTACAGTTGTTTCTTCTTCTCGA | | |
| | CAGATAAGGGTACATTTTTGTGCTGATAAAGTCAATGCTGCAAGCCGATTTAATGCTACT | | |
| | TACCAAGTAGATGGGTTCTGTTTGCCATGGGAAATACCCTGTGGAGGTAACTGGGGGTGT | | |
| | TATACTGAGCAGCAGCGTTGTGATGGGTATTGGCATTGCCCAAATGGAAGGGATGAAACC | | |
| | AATTGTACCATGTGCCAGAAGGAAGAATTTCCATGTTCCCGAAATGGTGTCTGTTATCCT | | |
| | CGTTCTGATCGCTGCAACTACCAGAATCATTGCCCAAATGGCTCAGATGAAAAAAACTGC | | |
| | TTTTTTTGCCAACCAGGAAATTTCCATTGTAAAAACAATCGTTGTGTGTTTGAAAGTTGG | | |
| | GTGTGTGATTCTCAAGATCACTGTGGTGATGCCAGCGATGAAGAAAATTCCCCAGTAATC | | |
| | GTGCCTACAAGAGTCATCACTGCTGCCGTCATAGGGAGCCTCATCTGTGGCCTGTTACTC | | |
| | GTCATAGCATTGGGATGTACTTGTAAGCTTTATTCTCTGAGAATGTTTGAAAGAAGATCA | | |
| | TTTGAAACACAGTTGTCAAGAGTGGAAGCAGAATTGTTAAGAAGACAAGCTCCTCCCTCG | | |
| | TATGGACAATTGATTGCTCAGGGTTTAATTCCACCAGTTGAAGATTTTCCTGTTTCTTCA | | |
| | CCTAATCAGGCTTCTGTTTTGGAAAATCTGAGGCTAGCCGTACGATCTCAGCTTGGATTT | | |
| | ACTTCAGTCAGGCTTCCTATGGCAGGCAGATCAAGCAACATTTGGAACCGTATTTTTAAT | | |
| | TTTGCAAGATCACGTCATTCTGGGTCATTGGCTTTGGTCTCAGCAGATGGAGATGAGGTT | | |
| | GTCCCTAGTCAGAGTACCACTAGAGAACCTGAGAGAAATCATACTCACAGAAGTTTGTTT | | |
| | TCCGTGGAGTCTGATGATACAGACACAGAAAATGAGAGAAGAGATATGGCAGGAGCATCT | | |
| | GGTGGGGTTGCAGCTCCTTTGCCTCAAAAAGTCCCTCCCACAACGGCAGTGGAAGCGACA | | |
| | GTAGGAGCATGTCCAAGTTCCTCAACTCAGAGTACCCGAGGTGGTCATGCAGATAATGGA | | |
| | AGGGATGTGACAAGTGTGGAACCCCCAAGTGTGAGTCCAGCACGTCACCAGCTTACAAGT | | |
| | GCACTCAGTCGTATGACTCAGGGGCTACGCTCGGTACGTTTTACATTAGGACGATCAAGT | | |
| | TCCCTAAGTCAGAACCAGAGTCCTTTGAGACAACTTGATAATGGCGTAAGTGGAAGAGAA | | |
| | GATGATGATGATGTTGAAATGCTAATTCCAATTTCTGATGGATCTTCAGACTTTGATGTG | | |
| | AATGACTGCTCCAGACCTCTTCTTGATCTTGCCTCAGATCAAGGACAAGGGCTTAGACAA | | |
| | CCATATAATGCAACAAATCCTGGAGTAAGGCCAAGTAATCGAGATGGCCCCTGTGAGCGC | | |
| | TGTCGTATTGTCCACACTGCCCAGATACCAGACACTTGCTTAGAAGTAACACTGAAAAAC | | |
| | GAAACGAGTGATGATGAGGCTTTGTTACTTTGTTAGGTACGAATCACATAACGGAGATTG | | |
| | TATACAAGTTGGAGCAATATCCATTTATTATTTTGTAACTTTACAGTTAAACTAGTTTTA | | |
| | GTTTAAAAAGAAAAAATGCAGGGTGATTTCTTATTATTATATGTTAGCCTGCATGGTTAA | | |
| | ATTCGACAACTTGTAACTCTATGAACTTAGAGTTTACTATTTTAGCAGCTAAAAATGCAT | | |
| | CACATATTGCATATTGTTCAATAATGGTCCTTTCATTTGTTTCTGATTGTTTTCATCCTG | | |
| | ATACTGTAGTTCACTGTAGAAATGTGGCTGCTGAAACTCATTTGATTGTCATTTTTATCT | | |
| | ATCCTATGTTAAATGGTTTGTTTTTACAAAATAATACCTTATTTTAATTGAAACCTTTAT | | |
| | GCTTTTGCCAAGCACATCTTGTAACTTAATATAGCTAGATGTTAAGGTTGTTAATGTACC | | |
| | AAAAAAAAAAAA | | |
| | ORF Start: ATG at 43 | | ORF Stop: TAG at 2554 |
| | SEQ ID NO: 132 | 837 aa | MW at 92869.5kD |
| NOV31e, | MACRWSTKESPRWRSALLLLFLAGVYAVRTQQYSTSGIITSPGWPSEYPAKINCSWFIRA | | |
| CG51264-07 | | | |
| Protein Sequence | NPGEIITISFQDFDIQGSRRCNLDWLTIETYKNIESYRACGSTIPPPYISSQDHIWIRFH | | |
| | SDDNISRKCFRLAYLSGKSEEPNCACDQFRCGNGKCIPEAWKCNNMDECGDSSDEEICAK | | |
| | EANPPTAAAFQPCAYNQFQCLSRFTKVYTCLPESLKCDGNIDCLDLCDEIDCDVPTCGQW | | |
| | LKYFYGTFNSPNYPDFYPPGSNCTWLIDTGDHRKVILRFTDFKLDGTGYGDYVKIYDCLE | | |
| | ENPHKLLRVLTAFDSHAPLTVVSSSCQTRVHFCADKVNAARGFNATYQVDGFCLPWEIPC | | |
| | GGNWGCYTEQQRCDGYWHCPNGRDETNCTMCQKEEFPCSRNGVCYPRSDRCNYQNHCPNG | | |
| | SDEKNCFFCQPGNFHCKNNRCVFESWVCDSQDDCGDGSDEENCPVIVPTRVITAAVIGSL | | |
| | ICGLLLVIALGCTCKLYSLRMFERRSFETQLSRVEAELLRREAPPSYGQLIAQGLIPPVE | | |
| | DFPVCSPNQASVLENLRLAVRSQLGFTSVRLPMAGRSSNIWNRIFNFARSRHSGSLALVS | | |
| | ADGDEVVPSQSTSREPERNHTHRSLFSVESDDTDTENERRDMAGASGGVAAPLPQKVPPT | | |
| | TAVEATVGACASSSTQSTRGGHADNGRDVTSVEPPSVSPARHQLTSALSRMTQGLRWVRF | | |
| | TLGRSSSLSQNQSPLRQLDNGVSCREDDDDVEMLIPISDGSSDFDVNDCSRPLLDLASDQ | | |
| | GQGLRQPYNATNPGVRPSNRDGPCERCGIVHTAQIPDTCLEVTLKNETSDDEALLLC | | |
| | SEQ ID NO: 133 | 1441 bp | |
| NOV31f, | CGCCGGTGGCTCGGCCGCGCCGGCGGCCGCGGCGCCGGCGGCGGCGGCCGCGTCGTCTAC | | |
| CG51264-02 | | | |
| DNA Sequence | CTCCAGCTTCTCCTCCCTCCTCCTCCCTCTCCTCCTCTCTCTCTCCATCTGCTGTCGTTA | | |
| | TGGCCTGTCGCTGCAGCACAAAAGAGTCTCCGCGGTGGAGGTCTGCGTTGCTCTTGCTTT | | |
| | TCCTCGCTGGCGTGTACGCTTGTGGAGAGACTCCAGAGCAAATACGAGCACCAAGTCGCA | | |
| | TAATCACAAGCCCAGGCTGGCCTTCTGAATATCCTGCAAAAATCAACTGTACCTGGTTCA | | |
| | TAAGCGCAAACCCAGGCGAAATCATTACTATAAGTTTTCAGGATTTTGATATTcAAGCAT | | |
| | CCAGAAGGTGCAATTTGGACTGGTTGACAATAGAAACATACAAGAATATTGAAAGTTACA | | |
| | GAGCTTGTGGTTCCACAATTCCACCTCCGTATATCTCTTCACAAGACCACATCTGCATTA | | |
| | GGTTTCATTCGGATGACAACATCTCTACAAAGCGTTTCACACTGCCATATTTTTCAGGGA | | |
| | AATCTGAGGAACCAAATTGTGCTTGTGATCAGTTTCGTTGTGGTAATGGAAAGTGTATAC | | |
| | CAGAAGCCTGGAAATGTAATAACATGCATGAATCTGGAGATAGTTCCGATGAAGAGATCT | | |
| | GTGCCAAAGAACCAAATCCTCCAACTGCTCCTGCTTTTCAACCCTGTGCTTACAACCAGT | | |
| | TCCAGTGTTTATCCCGTTTTACCAAAGTTTACACTTGCCTCCCCGAATCTTTAAAATGTG | | |
| | ATGGGAACATTGACTGCCTTGACCTAGGAGATGAGATAGACTGTGATGTGCCAACATGTG | | |
| | GGCAATCCCTAAAATATTTTTATGGTACTTTTAATTCTCCCAATTATCCACACTTTTATC | | |
| | CTCCTGCAAGCAATTGCACCTCGTTAATAGACACTCGTGATCACCGTAAAGTCATTTTAC | | |
| | GCTTCACTGACTTTAAACTTGATGGTACTGGTTATCGTGATTATGTCAAAATATATCATG | | |
| | GATTAGACCAGAATCCACACAAGCTTTTGCGTGTGTTGACAGCTTTTGATTCTCATGCAC | | |
| | CTCTTACAGTTGTTTCTTCTTCTGGACAGATAAGCGTACATTTTTGTGCTGATAAAGTGA | | |
| | ATGCTGCAAGGGGATTTAATGCTACTTACCAAGTAGATGGGTTCTGTTTGCCATGGGAAA | | |
| | TACCCTGTGCACGTAACTCGGCGTCTTATACTCACCACCAGCGTCGTGATGGGTATTGGC | | |
| | ATTGCCCAAATGGAAGGGATGAAACCAATTGTACCATGTGCCAGAAGGAAGAATTTCCAT | | |
| | GTTCCCGAAATGGTGTCTGCTATCCTCGCTCTGATCGCTGCAACTACCAGAATCATTGCC | | |
| | CAAATGGCAAACAGAACCCATCTACTTGGTAAGTAGCATTAAATCCCCTTGCAGCATTCA | | |
| | C | | |
| | ORF Start: at 3 | | ORF Stop: TAA at 1410 |
| | SEQ ID NO: 134 | 469 aa | MW at 53338.2kD |
| NOV31f | PVARRRRRRRRRRRRRRRLPPASPPSSSVSSSLSPSAVVMACRWSTKESPRWRSALLLLF | | |
| CG51264-02 | | | |
| Protein Sequence | LAGVYACGETPEQIRAPSGIITSPCWPSEYPAKINCSWFIRANPCEIITISFQDFDIQGS | | |
| | RRCNLDWLTIETYKNIESYRACGSTIPPPYISSQDHIWIRFHSDDNISRKGFRLAYFSGK | | |
| | SEEPNCACDQFRCGNGKCIPEAWKCNNMDECGDSSDEEICAKEANPPTAAAFQPCAYNQF | | |
| | QCLSRFTKVYTCLPESLKCDGNIDCLDLGDEIDCDVPTCGQWLKYFYGTFNSPNYPDFYP | | |
| | PGSNCTWLIDTGDHRKVILRFTDFKLDGTGYGDYVKIYDGLEENPHKLLRVLTAFDSHAP | | |
| | LTVVSSSGQIRVHFCADKVNAARGFNATYQVDGFCLPWEIPCGGNWGCYTEQQRRDGYWH | | |
| | CPNGRDETNCTMCQKEEFPCSRNGVCYPRSDRCNYQNHCPNGKQNPSTW | | |
| | SEQ ID NO: 135 | 3078 bp | |
| NOV31g, | CTCCTCCTCCGTCTCCTCCTCTCTCTCTCATCTGCTGTGGTTATGGCCTGTCGCTGGAGC | | |
| CG51264-05 | | | |
| DNA Sequence | ACAAAAGAGTCTCCGCGGTGGAGGTCTGCGTTGCTCTTGCTTTTCCTCGCTGGGGTGTAC | | |
| | GGAAATGGTGCTCTTGCAGAACATTCTGAAAATGTGCATATTTCAGGAGTGTCAACTCCT | | |
| | TGTGGAGAGACTCCAGAGCAAATACGAGCACCAAGTGGCATAATCACAAGCCCAGGCTGG | | |
| | CCTTCTGAATATCCTGCAAAAATCAACTGTAGCTGGTTCATAAGGGCAAACCCAGGCGAA | | |
| | ATCATTACTATAAGTTTTCACCATTTTGATATTCAAGGATCCAGAAGGTGCAATTTGGAC | | |
| | TGGTTGACAATAGAAACATACAAGAATATTGAAAGTTACAGAGCTTGTGGTTCCACAATT | | |
| | CCACCTCCGTATATCTCTTCACAAGACCACATCTGGATTACCTTTCATTCGGATGACAAC | | |
| | ATCTCTAGAAAGGGTTTCAGACTGGCATATTTTTCAGGGAAATCTGAGGAACCAAATTGT | | |
| | GCTTGTGATCAGTTTCGTTGTGGTAATGGAAAGTGTATACCAGAAGCCTGGAAATGCAAT | | |
| | AACATGGATGAATGTGGAGATAGTTCCGATGAAGAGATCTGTGCCAAAGAAGCAAATCCT | | |
| | CCAACTGCTGCTGCTTTTCAACCCTGTGCTTACAACCAGTTCCAGTGTTTATCCCCTTTT | | |
| | ACCAAAGTTTACACTTGCCTCCCCGAATCTTTAAAATGTGATGGGAACATTGACTGCCTT | | |
| | GACCTAGGAGATGAGATAGACTGTGATGTGCCAACATGTGGGCAATGGCTAAAATATTTT | | |
| | TATGGTACTTTTAATTCTCCCAATTATCCAGACTTTTATCCTCCTGGAAGCAATTGCACC | | |
| | TGGTTAATAGACACTGGTGATCACCGTAAAGTCATTTTACGCTTCACTGACTTTAAACTT | | |
| | GATGGTACTGGTTATGGTGATTATGTCAAAATATATGATGGATTAGAGGAGAATCCACAC | | |
| | AAGCTTTTGCGTCTGTTGACAGCTTTTGATTCTCATGCACCTCTTACAGTTGTTTCTTCT | | |
| | TCTGGACAGATAAGGGTACATTTTTGTGCTGATAAAGTGAATGCTGCAAGGGGATTTAAT | | |
| | GCTACTTACCAAGTAGATGGGTTCTGTTTGCCATGGGAAATACCCTGTGGAGGTAACTGG | | |
| | GGGTGTTATACTGAGCAGCAGCGTTGTGATGGGTATTGGCATTGCCCAAATGGAAGGGAT | | |
| | GAAACCAATTGTACCATGTGCCAGAAGGAAGAATTTCCATGTTCCCGAAATGGTGTCTGT | | |
| | TATCCTCGTTCTGATCGCTGCAACTACCAGAATCATTGCCCAAATGGCTCAGATGAAAAA | | |
| | AACTGCTTTTTTTGCCAACCAGGAAATTTCCATTGTAAAAACAATCGTTGTGTGTTTGAA | | |
| | AGTTGGGTGTGTGATTCTCAAGATGACTGTGGTGATGGCAGCGATGAAGAAAATTGCCCA | | |
| | GTAATCGTGCCTACAAGAGTCATCACTGCTCCCGTCATAGGCAGCCTCATCTGTGGCCTG | | |
| | TTACTCGTCATAGCATTGGGATGTACTTGTAAGCTTTATTCTCTGAGAATGTTTGAAAGA | | |
| | AGATCATTTGAAACACAGTTGTCAAGAGTGGAAGCAGAATTGTTAAGAAGAGAAGCTCCT | | |
| | CCCTCGTATGGACAATTGATTGCTCAGGGTTTAATTCCACCAGTTGAAGATTTTCCTGTT | | |
| | TGTTCACCTAATCAGGCTTCTGTTTTGGAAAATCTGAGGCTAGCCCTACGATCTCAGCTT | | |
| | GGATTTACTTCAGTCAGGCTTCCTATGGCAGGCAGATCAAGCAACATTTGGAACCGTATT | | |
| | TTTAATTTTGCAAGATCACGTCATTCTGGGTCATTGGCTTTGGTCTCAGCAGATGGAGAT | | |
| | GAGGTTGTCCCTAGTCAGAGTACCAGTAGAGAACCTGAGAGAAATCATACTCACAGAAGT | | |
| | TTGTTTTCCGTGGAGTCTGATGATACAGACACAGAAAATGAGAGAAGAGATATGGCAGGA | | |
| | GCATCTGGTGGGGTTGCAGCTCCTTTGCCTCAAAAAGTCCCTCCCACAACGGCAGTGGAA | | |
| | GCGACAGTAGGAGCATGTGCAAGTTCCTCAACTCACAGTACCCGAGGTCCTCATCCAGAT | | |
| | AATGGAACGGATGTGACAAGTGTGGAACCCCCAAGTGTGACTCCAGCACGTCACCAGCTT | | |
| | ACAAGTGCACTCACTCGTATGACTCAGGGGCTACGCTGGGTACGTTTTACATTAGGACGA | | |
| | TCAAGTTCCCTAAGTCAGAACCAGAGTCCTTTGAGACAACTTGATAATGGGGTAAGTGGA | | |
| | AGAGAAGATGATGATGATGTTGAAATGCTAATTCCAATTTCTGATGGATCTTCAGACTTT | | |
| | GATGTGAATGACTGCTCCAGACCTCTTCTTGATCTTGCCTCAGATCAAGGACAAGGGCTT | | |
| | AGACAACCATATAATGCAACAAATCCTGGAGTAAGGCCAAGTAATCGAGATGGCCCCTGT | | |
| | GAGCGCTGTGGTATTGTCCACACTGCCCAGATACCAGACACTTGCTTAGAAGTAACACTG | | |
| | AAAAACGAAACGAGTGATGATGAGGCTTTGTTACTTTGTTAGGTACGAATCACATAAGGG | | |
| | AGATTGTATACAAGTTGGAGCAATATCCATTTATTATTTTGTAACTTTACAGTTAAACTA | | |
| | GTTTTAGTTTAAAAAGAAAAAATGCAGGGTGATTTCTTATTATTATATGTTAGCCTCCAT | | |
| | GGTTAAATTCGACAACTTGTAACTCTATGAACTTAGAGTTTACTATTTTAGCAGCTAAAA | | |
| | ATGCATCACATATTGCATATTGTTCAATAATGGTCCTTTCATTTGTTTCTGATTGTTTTC | | |
| | ATCCTGATACTGTAGTTCACTGTAGAAATGTGGCTGCTGAAACTCATTTGATTGTCATTT | | |
| | TTATCTATCCTATGTTAAATGGTTTGTTTTTACAAAATAATACCTTATTTTAATTGAAAC | | |
| | GTTTATGCTTTTGCCAAGCACATCTTGTAACTTAATATAGCTAGATGTTAAGGTTGTTAA | | |
| | TGTACCAAAAAAAAAAAA | | |
| | ORF Start: ATG at 43 | | ORF Stop: TAG at 2620 |
| | SEQ ID NO: 136 | 859 aa | MW at 94982.7kD |
| NOV31g, | MACRWSTKESPRWRSALLLLFLAGVYGNGALAEHSENVHISGVSTACGETPEQIRAPSGI | | |
| CG51264-05 | | | |
| Protein Sequence | ITSPGWPSEYPAKINCSWFIRANPGEIITISFQDFDIQGSRRCNLDWLTIETYKNIESYR | | |
| | ACGSTIPPPYISSQDHIWIRFHSDDNISRKGFRLAYFSGKSEEPNCACDQFRCGNGKCIP | | |
| | EAWKCNNMDECGDSSDEEICAKEANPPTAAAFQPCAYNQFQCLSRFTKVYTCLPESLKCD | | |
| | GNIDCLDLGDEIDCDVPTCGQWLKYFYGTFNSPNYPDFYPPGSNCTWLIDTGDHRKVILR | | |
| | FTDFKLDGTGYGDYVKIYDGLEENPHKLLRVLTAFDSHAPLTVVSSSGQIRVHFCADKVN | | |
| | AARGFNATYQVDGFCLPWEIPCGGNWGCYTEQQRCDGYWHCPNGRDETNCTMCQKEEFPC | | |
| | SRNGVCYPRSDRCNYQNHCPNGSDEKNCFFCQPGNFHCKNNRCVFESWVCDSQDDCGDGS | | |
| | DEENCPVIVPTRVITAAVICSLICGLLLVIALGCTCKLYSLRMFERRSFETQLSRVEAEL | | |
| | LRREAPPSYGQLIAQGLIPPVEDFPVCSPNQASVLENLRLAVRSQLGFTSVRLPMAGRSS | | |
| | NIWNRIFNFARSRHSGSLALVSADGDEVVPSQSTSREPERNHTHRSLFSVESDDTDTENE | | |
| | RRDMAGASGGVAAPLPQKVPPTTAVEATVGACASSSTQSTRGGHADNGRDVTSVEPPSVS | | |
| | PARHQLTSALSRMTQGLRWVRFTLGRSSSLSQNQSPLRQLDNGVSGREDDDDVEMLIPIS | | |
| | DGSSDFDVNDCSRPLLDLASDQCQGLRQPYNATNPGVRPSNRDGPCERCCIVHTAQIPDT | | |
| | CLEVTLKNETSDDEALLLC | | |
| | SEQ ID NO: 137 | 1389 bp | |
| NOV31b, | AATGGTGCTCTTGCAGAACATTCTGAAAATGTGCATATTTCACGAGTGTCAACTCCTTGT | | |
| CG51264-08 | | | |
| DNA Sequence | GGAGAGACTCCAGAGCAAATACGAGCACCAAGTGGCATAATCACAACCCCAGGCTGGCCT | | |
| | TCTGAATATCCTGCAAAAACCAACTGTAGCTGGTTCATAAGGGCAAACCCAGCCGAAATC | | |
| | ATTACTATAACTTTTCAGGATTTTGATATTCAAGGATCCAGAAGGTCCAATTTCGACTGC | | |
| | TTGACAATAGAAACATACAAGAATATTGAAAGTTACAGACCTTGTGGTTCCACAATTCCA | | |
| | CCTCCGTATATCTCTTCACAAGACCACATCTGGATTAGGTTTCATTCGGATGACAACATC | | |
| | TCTACAAAGGGTTTCAGACTGGCATATTTTTCAGGGAAATCTGAGGAACCAAATTGTGCT | | |
| | TGTGATCAGTTTCGTTGTGGTAATGGAAAGTGTATACCAGAAGCCTGGAAATGTAATAAC | | |
| | ATGGATGAATGTGGAGATAGTTCCGATGAAGAGATCTGTGCCAAAGAACCAAATCCTCCA | | |
| | ACTGCTGCTGCTTTTCAACCCTGTGCTTACAACCAGTTCCAGTGTTTATCCCGTTTTACC | | |
| | AAAGTTTACACTTGCCTCCCCGAATCTTTAAAATGTGATGGCAACATTGACTGCCTTGAC | | |
| | CTACGAGATGAGATAGACTGTGATGTCCCAACATGTGGGCAATGGCTAAAATATTTTTAT | | |
| | GGTACTTTTAATTCTCCCAATTATCCAGACTTTTATCCTCCTCGAAGCAATTGCACCTGG | | |
| | TTAATAGACACTGGTGATCACCGTAAAGTCATTTTACGCTTCACTGACTTTAAACTTGAT | | |
| | GGTACTGGTTATGGTGATTATCTCAAAATATATGATGGATTAGAGGACAATCCACACAAG | | |
| | CTTTTGCGTGTGTTGACAGCTTTTGATTCTCATGCACCTCTTACAGTTGTTTCTTCTTCT | | |
| | GGACAGATAAGGGTACATTTTTGTGCTGATAAAGTGAATGCTGCAAGGGCATTTAATGCT | | |
| | ACTTACCAAGTAGATGGGTTCTGTTTCCCATGGGAAATACCCTGTGCAGGTAACTGGGGG | | |
| | TGTTATACTGAGCAGCAGCGTTGTGATGGCTATTGGCATTGCCCAAATGGAAGGGATGAA | | |
| | ACCAATTGTACCATGTGCCAGAAGGAAGAATTTCCATGTTCCCGAAATGGTGTCTGTTAT | | |
| | CCTCGTTCTGATCGCTCCAACTACCAGAATCATTGCCCAAATGGCTCAGATGAAAAPAAC | | |
| | TGCTTTTTTTGCCAACCAGGAAATTTCCATTGTAAAAACAATCGTTGTGTGTTTGAAAGT | | |
| | TGGGTGTGTGATTCTCAAGATGACTGTGGTGATGGCAGCCATGAAGAAAATTGCCCAGTA | | |
| | ATCGTGCCT | | |
| | ORF Start: at 1 | | ORF Stop: end of sequence |
| | SEQ ID NO: 138 | 463 aa | MW at 52053.1kD |
| NOV31h, | NGALAEHSENVHISGVSTACGETPEQIRAPSGIITSPGWPSEYPAKTNCSWFIRANPGEI | | |
| CG51264-08 | | | |
| Protein Sequence | ITISFQDFDIQGSRRCNLDWLTIETYKNIESYRACGSTILPPPYISSQHIWIRFHSDDNI | | |
| | SRKGFRLAYPSGKSEEPNCACDQFRCGNGKCIPEAWKCNNMDECGDSSDEEICAKEANPP | | |
| | TAAAFQPCAYNQFQCLSRFTKVYTCLPESLKCDGNIDCLDLGDEIDCDVPTCGQWLKYFY | | |
| | GTFNSPNYPDFYPPGSNCTWLIDTGDHRKVILRFTDFKLDGTGYGDYVKIYDGLEENPHK | | |
| | LLRVLTAFDSHAPLTVVSSSGQIRVHFCADKVNAARGFNATYQVDGFCLPWEIPCGGNWG | | |
| | CYTEQQRCDGYWHCPNGRDETNCTMCQKEEFPCSRNGVCYPRSDRCNYQNHCPNGSDEKN | | |
| | CFFCQPGNFHCKNNRCVFESWVCDSQDDCGDGSDEENCPVIVP | | |
| | SEQ ID NO: 139 | 1389 bp | |
| NOV31i, | AATGGTGCTCTTGCAGAACATTCTGAAAATGTGCATATTTCAGGAGTGTCAACTGCTTGT | | |
| CG51264-09 | | | |
| DNA Sequence | GGAGAGACTCCAGAGCAAATACGAGCACCAAGTGGCATAATCACAAGCCCAGGCTGGCCT | | |
| | TCTGAATATCCTGCAAAAACCAACTGTAGCTGGTTCATAAGGGCAAACCCAGGCGAAATC | | |
| | ATTACTATAAGTTTTCAGGATTTTGATATTCAAGGATCCAGAAGGTGCAATTTGGACTGG | | |
| | TTGACAATAGAAACATACAAGAATATTGAAAGTTACAGAGCTTGTGGTTCCACAATTCCA | | |
| | CCTCCGTATATCTCTTCACAAGACCACATCTGGATTAGGTTTCATTCGGATCACAACATC | | |
| | TCTAGAAAGGGTTTCAGACTGGCATATTTTTCAGGGAAATCTGAGGAACCAAATTGTGCT | | |
| | TGTGATCAGTTTCGTTGTGGTAATGGAAAGTGTATACCAGAAGCCTGGAAATGTAATAAC | | |
| | ATGGATGAATGTGGAGATAGTTCCGATGAAGAGATCTGTGCCAAAGAAGCAAATCCTCCA | | |
| | ACTGCTGCTGCTTTTCAACCCTGTGCTTACAACCAGTTCCAGTGTTTATCCCGTTTTACC | | |
| | AAAGTTTACACTTGCCTCCCCGAATCTTTAAAATGTGATGGGAACATTGACTGCCTTGAC | | |
| | CTAGCAGATGAGATAGACTGTGATGTGCCAACATGTGGGCAATGGCTAAAATATTTTTAT | | |
| | GGTACTTTTAATTCTCCCAATTATCCAGACTTTTATCCTCCTGGAAGCAATTGCACCTGG | | |
| | TTAATAGACACTGGTGATCACCGTAAAGTCATTTTACGCTTCACTGACTTTAAACTTGAT | | |
| | GGTACTGGTTATGGTGATTATGTCAAAATATATGATGGATTAGAGGAGAATCCACACAAG | | |
| | CTTTTGCGTGTGTTGACAGCTTTTGATTCTCATGCACCTCTTACAGTTGTTTCTTCTTCT | | |
| | GGACAGATAAGGGTACATTTTTGTGCTGATAAAGTGAATGCTGCAAGGGGATTTAATGCT | | |
| | ACTTACCAAGTAGATGGGTTCTGTTTGCCATGGGAAATACCCTGTGGAGGTAACTGGGGG | | |
| | TGTTATACTGAGCAGCAGCGTTGTGATGGGTATTGGCATTGCCCAAATGGAAGGGATGAA | | |
| | ACCAATTGTACCATGTGCCAGAAGGAAGAATTTCCATGTTCCCGAAATGGTGTCTGTTAT | | |
| | CCTCGTTCTGATCGCTGCAACTACCAGAATCATTGCCCAAATGGCTCAGATGAAAAAAAC | | |
| | TGCTTTTTTTGCCAACCAGCAAATTTCCATTGTAAAAACAATCGTTGTGTGTTTGAAAGT | | |
| | TGGGTGTGTGATTCTCAAGATGACTGTGGTGATGGCAGCGATGAAGAAAATTGCCCAGTA | | |
| | ATCGTGCCT | | |
| | ORF Start: at 1 | | ORF Stop: end of sequence |
| | SEQ ID NO: 140 | 463 aa | MW at 52053.1kD |
| NOV31i, | NGALAEHSENVHISGVSTACGETPEQIRAPSGIITSPGWPSEYPAKTNCSWPIRANPGEI | | |
| CG51264-09 | | | |
| Protein sequence | ITISFQDFDIQGSRRCNLDWLTIETYKNIESYRACGSTIPPPYISSQDHIWIRFHSDDNI | | |
| | SRKGFRLAYFSGKSEEPNCACDQFRCGNGKCIPEAWKCNNMDECGDSSDEEICAKEANPP | | |
| | TAAAFQPCAYNQFQCLSRFTKVYTCLPESLKCDGNIDCLDLGDEIDCDVPTCGQWLKYFY | | |
| | GTFNSPNYPDFYPPGSNCTWLIDTGDHRKVILRFTDFKLDGTGYGDYVKIYDGLEENPHK | | |
| | LLRVLTAFDSHAPLTVVSSSGQIRVHFCADKVNAARGFNATYQVDGFCLPWEIPCGGNWG | | |
| | CYTEQQRCDGYWHCPNCRDETNCTMCOKEEFPCSRNGVCYPRSDRCNYQNHCPNGSDEKN | | |
| | CFFCQPGNFHCKNNRCVFESWVCDSQDDCGDGSDEENCPVIVP | | |
| | SEQ ID NO: 141 | 1401 bp | |
| NOV31j, | GGTACCAATGCTGCTCTTGCAGAACATTCTGAAAATGTGCATATTTCAGGAGTGTCAACT | | |
| CG51264-10 | | | |
| DNA Sequence | ACTTGTCCAGAGACTCCAGGGCAAATACGAGCACCAAGTGGCATAATCACAAGCCCAGGC | | |
| | TGGCCTTCTCAATATCCTGCAAAAATCAACTGTAGCTCGTTCATAAGGGCAAACCCAGGC | | |
| | GAAATCATTACTATAAGTTTTCAGGATTTTGATATTCAAGGATCCAGAAGGTGCAATTTG | | |
| | GACTGGTTGACAATAGAAACATACAAGAATATTGAAAGTTACAGAGCTTGTGGTTCCACA | | |
| | ATTCCACCTCCGTATATCTCTTCACAAGACCACATCTGGATTAGGTTTCATTCGGATGAC | | |
| | AACATCTCTAGAAACGGTTTCAGACTGGCATATTTTTCAGGGAAATCTGAGGAACCAAAT | | |
| | TGTGCTTCTGATCAGTTTCGTTGTGGTAATGGAAAGTGTATACCAGAAGCCTCGAAATGT | | |
| | AATAACATGGATGAATGTGGAGATAGTTCCGATGAAGAGATCTGTGCCAAAGAAGCAAAT | | |
| | CCTCCAACTGCTGCTGCTTTTCAACCCTGTGCTTACAACCAGTTCCAGTGTTTATCCCGT | | |
| | TTTACCAAAGTTTACACTTCCCTCCCCGAATCTTTAAAATGTGATGGGAACATTGACTGC | | |
| | CTTGACCTAGGAOATCAGATAGACTGTGATGTGCCAACATGTGGGCAATGGCTAAAATAT | | |
| | TTTTATGGTACTTTTAATTCTCCCAATTATCCACACTTTTATCCTCCTGGAAGCAATTGC | | |
| | ACCTGGTTAATAGACACTGGTGATCACCGTAAAGTCATTTTACGCTTCACTGACTTTAAA | | |
| | CTTGATGCTACTGGTTATGGTGATTATGTCAAAATATATGATGGATTAGAGGAGAATCCA | | |
| | CACAACCTTTTCCGTGTGTTGACAGCTTTTGATTCTCATGCACCTCTTACAGTTGTTTCT | | |
| | TCTTCTGGACAGATAAGGGTACATTTTTGTGCTGATAAAGTGAATGCTGCAAGGCGATTT | | |
| | AATGCTACTTACCAAGTAGATGGGTTCTGTTTGCCATGGGAAATACCCTGTGGAGGTAAC | | |
| | TGGGGGTGTTATACTGAGCAGCAGCGTTGTGATGGGTATTGGCATTGCCCAAATGGAAGG | | |
| | GATGAAACCAATTGTACCATGTGCCAGAAGGAAGAATTTCCATGTTCCCGAAATGGTGTC | | |
| | TGTTATCCTCGTTCTGATCGCTGCAACTACCAGAATCATTGCCCAAATGGCTCAGATGAA | | |
| | AAAAACTGCTTTTTTTGCCAACCAGGAAATTTCCATTGTAAAAACAATCGTTGTGTGTTT | | |
| | GAAAGTTGGGTGTGTGATTCTCAAGATGACTGTGGTGATGGCAGCGATGAAGAAAATTGC | | |
| | CCAGTAATCGTGCCTCGGCCG | | |
| | ORF Start: at 7 | | ORF Stop: at 1396 |
| | SEQ ID NO: 142 | 463 aa | MW at 52023.1kD |
| NOV31j, | NGALAEHSENVHISGVSTTCGETPGQIRAPSGIITSPGWPSEYPAKINCSWFIRANPGEI | | |
| CG51264-10 | | | |
| Protein Sequence | ITISFQDFDIQGSRRCNLDWLTIETYKNIESYRACGSTIPPPYISSQDIIWIRFHSDDNI | | |
| | SRKGFRLAYFSGKSEEPNCACDQFRCGNGKCIPEAWKCNNMDECGDSSDEEICAKEANPP | | |
| | TAAAFQPCAYNQFQCLSRFTKVYTCLPESLKCDGNIDCLDLGDEIDCDVPTCGQWLKYFY | | |
| | GTFNSPNYPDFYPPGSNCTWLIDTGDHRKVILRFTDFKLDGTGYGDYVKIYDGLEENPHK | | |
| | LLRVLTAFDSHAPLTVVSSSGQIRVHFCADKVNAARGFNATYQVDGFCLPWEIPCGGNWG | | |
| | CYTEQQRCDGYWHCPNGRDETNCTMCQKEEFPCSRNGVCYPRSDRCNYQNHCPNGSDEKN | | |
| | CFPCQPGNFHCKNNRCVFESWVCDSQDDCGDCSDEENCPVIVP | | |
| | SEQ ID NO: 143 | 1401 bp | |
| NOV31k, | GGTACCAATGGTGCTCTTGCACAACATTCTGAAAATCTGCATATTTCAGGAGTGTCAACT | | |
| CG51264-11 | | | |
| DNA Sequence | GCTTGTGGAGAGACTCCAGAGCAAATACGAGCACCAAGTGGCATAATCACAAGCCCAGGC | | |
| | TGGCCTTCTCAATATCCTGCAAAAACCAACTGTAGCTGGTTCATAAGGGCAAACCCAGGC | | |
| | GAAATCATTACTATAAGTTTTCAGGATTTTGATATTCAAGGATCCAGAAGGTGCAATTTG | | |
| | GACTGGTTGACAATAGAAACATACAAGAATATTGAAAGTTACAGAGCTTGTGGTTCCACA | | |
| | ATTCCACCTCCGTATATCTCTTCACAAGACCACATCTGGATTAGGTTTCATTCGGATGAC | | |
| | AACATCTCTAGAAAGGGTTTCAGACTGGCATATTTTTCACCGAAATCTGAGGAACCAAAT | | |
| | TGTGCTTGTGATCAGTTTCGTTGTGGTAATGGAAAGTGTATACCAGAAGCCTGGAAATGT | | |
| | AATAACATGGATGAATGTGGAGATACTTCCGATGAAGAGATCTGTGCCAAAGAAGCAAAT | | |
| | CCTCCAACTGCTGCTGCTTTTCAACCCTGTGCTTACAACCAGTTCCAGTGTTTATCCCGT | | |
| | TTTACCAAAGTTTACACTTGCCTCCCCGAATCTTTAAAATGTGATGGGAACATTGACTGC | | |
| | CTTGACCTAGGAGATGAGATAGACTGTGATGTGCCAACATGTCGCCAATGCCTAAAATAT | | |
| | TTTTATGGTACTTTTAATTCTCCCAATTATCCAGACTTTTATCCTCCTCGAAGCAATTGC | | |
| | ACCTGGTTAATAGACACTGGTGATCACCGTAAAGTCATTTTACGCTTCACTGACTTTAAA | | |
| | CTTGATGGTACTGGTTATGGTGATTATGTCAAAATATATCATGGATTACAGGAGAATCCA | | |
| | CACAAGCTTTTGCGTGTGTTGACAGCTTTTGATTCTCATGCACCTCTTACAGTTGTTTCT | | |
| | TCTTCTGGACAGATAAGGGTACATTTTTGTGCTGATAAAGTGAATGCTCCAACGCGATTT | | |
| | AATGCTACTTACCAAGTAGATGGGTTCTGTTTGCCATGGGAAATACCCTGTGGAGGTAAC | | |
| | TGGGGGTGTTATACTGACCAGCAGCGTTGTGATGGGTATTGGCATTGCCCAAATGGAAGG | | |
| | GATGAAACCAATTGTACCATGTGCCAGAAGGAAGAATTTCCATGTTCCCGAAATGGTGTC | | |
| | TGTTATCCTCGTTCTGATCGCTGCAACTACCAGAATCATTCCCCAAATGGCTCAGATGAA | | |
| | AAAAACTGCTTTTTTTGCCAACCAGGAAATTTCCATTGTAAAAACAATCGTTGTGTGTTT | | |
| | GAAACTTGCGTGTGTGATTCTCAACATGACTGTGGTGATGGCAGCGATGAAGAAAATTGC | | |
| | CCAGTAATCGTGCCTCGGCCG | | |
| | ORF Start: at 7 | | ORF Stop: at 1396 |
| | SEQ ID NO: 144 | 463 aa | MW at 52053.1kD |
| NOV31k, | NGALAEHSENVHISGVSTACGETPEQIRAPSGIITSPCWPSEYPAKTNCSWFIRANPGEI | | |
| CG51264-11 | | | |
| Protein sequence | ITISFQDFDIQGSRRCNLDWLTIETYKNIESYRACGSTIPPPYISSQDHIWIRFHSDDNT | | |
| | SRKGFRLAYFSGKSEEPNCACDQFRCGNGKCIPEAWKCNNMDECGDSSDEEICAKEANPP | | |
| | TAAAFQPCAYNQFQCLSRFTKVYTCLPESLKCDGNIDCLDLCDEIDCDVPTCGQWLKYFY | | |
| | GTFNSPNYPDFYPPGSNCTWLIDTGDHRKVILRFTDFKLDGTGYGDYVKIYDCLEENPHK | | |
| | LLRVLTAFDSHAPLTVVSSSGQIRVHFCADKVNAARGFNATYQVDGFCLPWEIPCGGNWG | | |
| | CYTEQQRCDGYWHCPNGRDETNCTMCQKEEFPCSRNGVCYPRSDRCNYQNHCPNGSDEKN | | |
| | CFFCQPGNFHCKNNRCVFESWVCDSQDDCCDGSDEENCPVIVP | | |
| | SEQ ID NO: 145 | 1401 bp | |
| NOV31i, | GGTACCAATGGTCCTCTTGCAGAACATTCTGAAAATGTGCATATTTCAGGAGTGTCAACT | | |
| CG51264-12 | | | |
| DNA Sequence | GCTTGTGGAGAGACTCCAGAGCAAATACGAGCACCAAGTGGCATAATCACAAGCCCAGGC | | |
| | TGGCCTTCTGAATATCCTGCAAAAATCAACTGTAGCTGGTTCATAAGGGCAAACCCAGGC | | |
| | GAAATCATTACTATAAGTTTTCAGGATTTTGATATTCAAGGATCCAGAAGCTGCAATTTG | | |
| | GACTGGTTCACAATAGAAACATACAAGAATATTGAAAGTTACAGAGCTTGTGGTTCCACA | | |
| | ATTCCACCTCCGTATATCTCTTCACAAGACCACATCTGCATTAGGTTTCATTCGGATGAC | | |
| | AACATCTCTAGAAAGGGTTTCAGACTGGCATATTTTTCAGGGAAATCTGAGCAACCAAAT | | |
| | TGTGCTTGTGATCAGTTTCGTTGTCGTAATGGAAAGTGTATACCAGAAGCCTGGAAATGT | | |
| | AATAACATGGATGAATGTGGAGATAGTTCCGATGAAGAGATCTGTGCCAAAGAAGCAAAT | | |
| | CCTCCAACTGCTGCTGCTTTTCAACCCTGTGCTTACAACCAGTTCCAGTGTTTATCCCGT | | |
| | TTTACCAAAGTTTACACTTGCCTCCCCGAATCTTTAAAATGTGATGGCAACATTGACTGC | | |
| | CTTGACCTAGGAGATGAGATAGACTGTGATGTGCCAACATGTGGGCAATGGCTAAAATAT | | |
| | TTTTATGGTACTTTTAATTCTCCCAATTATCCAGACTTTTATCCTCCTGGAAGCAATTGC | | |
| | ACCTGGTTAATAGACACTGGTGATCACCGTAAAGTCATTTTACGCTTCACTGACTTTAAA | | |
| | CTTGATGGTACTGGTTATGGTGATTATGTCAAAATATATGATGGATTAGAGGAGAATCCA | | |
| | CACAAGCTTTTGCGTGTGTTGACAGCTTTTGATTCTCATGCACCTCTTACAGTTGTTTCT | | |
| | TCTTCTGGACAGATAAGGGTACATTTTTGTGCTGATAAAGTGAATGCTGCAAGGGGATTT | | |
| | AATGCTACTTACCAAGTAGATGGGTTCTGTTTGCCATGGGAAATACCCTGTGGAGGTAAC | | |
| | TGGGGGTGTTATACTGAGCAGCAGCGTTGTGATGGGTATTGGCATTGCCCAAATGGAAGG | | |
| | GATGAAACCAATTGTACCATGTGCCAGAAGGAAGAATTTCCATGTTCCCGAAATGGTGTC | | |
| | TGTTATCCTCGTTCTGATCGCTGCAACTACCAGAATCATTGCCCAAATGGCTCAGATGAA | | |
| | AAAAACTCCTTTTTTTGCCAACCAGGAAATTTCCATTGTAAAAACAATCGTTGTGTGTTT | | |
| | GAAAGTTGGGTGTGTGATTCTCAAGATGACTGTGGTGATGGCAGCGATGAAGAAAATTGC | | |
| | CCAGTAATCCTGCCTCGGCCG | | |
| | ORF Start: at 7 | | ORF Stop: at 1396 |
| | SEQ ID NO: 146 | 463 aa | MW at 52065.2kD |
| NOV31l, | NGALAEHSENVHISGVSTACGETPEQIRAPSGIITSPGWPSEYPAKINCSWFIRANPGEI | | |
| CG51264-12 | | | |
| Protein Sequence | ITISFQDFDTQGSRRCNLDWLTIETYKNTESYRACGSTIPPPYISSQDHIWIRFHSDDNI | | |
| | SRKGFRLAYFSGKSEEPNCACDQFRCCNGKCIPEAWKCNNMDECGDSSDEEICAKEANPP | | |
| | TAAAFQPCAYNQFQCLSRFTKVYTCLPESLKCDGNIDCLDLGDETDCDVPTCGQWLKYFY | | |
| | GTFNSPNYPDFYPPGSNCTWLIDTGDHRKVILRFTDFKLDGTCYGDYVKIYDGLEENPHK | | |
| | LLRVLTAFDSHAPLTVVSSSGQIRVMFCADKVNAARCFNATYQVDGFCLPWEIPCGGNWG | | |
| | CYTEQQRCDGYWHCPNGRDETNCTMCQREFPPCSRNGVCYPRSDRCNYQNHCPNGSDERN | | |
| | CFFCQPGNFHCKNNRCVPESWVCDSQDDCGDGSDEENCPVTVP | | |
| | SEQ ID NO: 147 | 1401 bp | |
| NOV31m, | GGTACCAATGGTGCTCTTGCAGAACATTCTGAAAAATGTGCATATTTCAGGAGTGTCAAC | | |
| CG51264-13 | | | |
| DNA Sequence | TGCTTCTGGAGAGACTCCAGAGCAAATACCACCACCAAGTGGCATAATCACAACCCCAG | | |
| | GCTCGCCTTCTCAATATCCTDCAAAAATCAACTGTAGCTGGTTCATAAGGGCAAACCCA | | |
| | GGCGAAATCATTACTATAAGTTTTCAGCATTTTCATATTCAAGGATCCAGAAGGTGCAA | | |
| | TTTGGACTCGTTCACAATAGAAACATACAAGAATATTGAAAGTTACAGAGCTTGTGGTT | | |
| | CTACAATTCCACCTCCGTATATCTCTTCACAACACCACATCTGGATTAGGTTTCATTCG | | |
| | GATGACAACATCTCTAGAAAGGCTTTCACACTGGCATATTTTTCAGGGAAATCTGACGA | | |
| | ACCAAATTGTGCTTGTGATCAGTTTCGTTGTGGTAATCGAAAGTCTATACCAGAACCCT | | |
| | GGAAATGTAATAACATGCATGAATGTGGAGATAGTTCCGATCAAGAGATCTGTGCCAAA | | |
| | GAAGCAAATCCTCCAACTGCTGCTCCTTTTCAACCCTGTGCTTACAACCACTTCCAGTG | | |
| | TTTATCCCGTTTTACCAAAGTTTACACTTGCCTCCCCGAATCTTTAAAATGTGATGCGA | | |
| | ACATTGACTGCCTTGACCTACGAGATOAGATACACTGTGATCTGCCAACATGTGGGCAA | | |
| | TGGCTAAAATATTTTTATGGTACTTTTAATTCTCCCAATTATCCACACTTTTATCCTCC | | |
| | TGGAAGCAATTGCACCTGGTTAATAGACACTGGTGATCACCGTAAAGTCATTTTACGCT | | |
| | TCACTGACTTTAAACTTCATGGTACTGGTTATGGTCATTATCTCAAAATATATGATGGA | | |
| | TTAGAGGAGAATCCACACAACCTTTTCCGTGTCTTGACAGCTTTTGATTCTCACGCACC | | |
| | TCTTACAGTTGTTTCTTCTTCTGGACAGATAAGGGTACATTTTTGTGCTGATAAAGTGA | | |
| | ATGCTGCAAGGGGATTTAATCCTACTTACCIAGTAGATGGGTTCTGTTTGCCATGGGAA | | |
| | ATACCCTGTGGAGGTAACTGGGGCTCTTATACTGAGCACCAGCGTTGTGATGGGTATTG | | |
| | GCATTGCCCAAATGCAAGGGATGAAACCAATTGTACCATGTGCCACAAGGAAGAATTTC | | |
| | CATGTTCCCGAAATGGTGTCTGTTATCCTCGTTCTGATCGCTGCAACTACCAGAATCAT | | |
| | TGCCCAAATGGCTCAGATCAAAAAAACTGCTTTTTTTGCCAACCACGAAATTTCCATTG | | |
| | TAAAAACAATCGTTGTCTGTTTGAAAGTTGGGTGTGTGATTCTCAAGATGACTGTGGTG | | |
| | ATGGCAGCGATGAACAAAATTGCCCAGTAATCGTGCCTCGGCCG | | |
| | ORF: Start at 7 | | ORF Stop: at 1396 |
| | SEQ ID NO: 148 | 463 aa | MW at 52065.2kD |
| NOV31m, | NGALAEHSENVHISGVSTACGETPEQIRAPSGIITSPGWPSEYPARINCSWFIRANPGE | | |
| CG51264-13 | | | |
| Protein Sequence | IITISFQDFDIQGSRRCNLDWLTIETYKNIESYRACGSTIPPPYISSQDHIWIRFHSDD | | |
| | NISRKGFRLAYFSGKSEEPNCACDQFRCGNCKCIPEAWKCNNMDECGDSSDEEICAKEA | | |
| | NPPTAAAFQPCAYNQFQCLSRFTKVYTCLPESLKCDGNIDCLDLGDEIDCDVPTCGQWL | | |
| | KYFYGTFNSPNYPDFYPPGSNCTWLIDTGDHRKVILRFTDFKLDGTGYGDYVKIYDGLE | | |
| | ENPHKLLRVLTAFDSHAPLTVVSSSGQIEVHFCADKVNAARGFNATYQVDGFCLPWEIP | | |
| | CGGNWGCYTEQQRCDGYWHCPNGRDETNCTMCQKEEFPCSRNGVCYPRSDRCNYQNHCP | | |
| | NGSDEKNCFFCQPGNFHCKNNRCVFESWVCDSQDDCGDGSDEENCPVIVP | | |
| | SEQ ID NO: 149 | 1401 bp | |
| NOV31n, | GGTACCAATGGTGCTCTTGCAGAACATTCTGAAAATGTGCATATTTCAGGAGTGTCAACT | | |
| CG51264-14 | | | |
| DNA Sequence | GCTTGTGGAGAGACTCCAGAGCAAATACCAGCACCAADTGGCATAATCACAAGCCCAGGC | | |
| | TGGCCTTCTGAATATCCTGCAAAAATCAACTGTAGCTGGTTCATAAGGGCAAACCCAGGC | | |
| | GAAATCATTACTATAAGTTTTCAGGATTTTGATATTCAAGGATCCAGAAGGTGCAATTTG | | |
| | GACTGGTTGACAATAGAAACATACAAGAATATTGAAAGTTACAGAGCTTGTGGTTCCACA | | |
| | ATTCCACCTCCGTATATCTCTTCACAAGACCACATCTGGATTAGGTTTCATTCGGATGAC | | |
| | AACATCTCTAGAAAGGGTTTCAGACTGGCATATTTTTCAGGGAAATCTGAGGAACCAAAT | | |
| | TGTGCTTGTGATCAGTTTCGTTGTGGTAATGGAAAGTGTATACCACAAGCCTGGAAATGT | | |
| | AATAACATGGATGAATGTGGAGATAGTTCCGATGAAGAGATCTGTGCCAAAGAAGCAAAT | | |
| | CCTCCAACTGCTGCTGCTTTTCAACCCTGTGCTTACAACCAGTTCCAGTGTTTATCCCGT | | |
| | TTTACCAAAGTTTACACTTGCCTCCCCGAATCTTTAAAATGTGATGGGAACATTGACTGC | | |
| | CTTGACCCAGGAGATGAGATAGACTGTCATGTGCCAACATGTGGGCAATGGCTAAAATAT | | |
| | TTTTATGGTACTTTTAATTCTCCCAATTATCCACACTTTTATCCTCCTOGAAGCAATTGC | | |
| | ACCTGGTTAATAGACACTGGTGATCACCGTAAAGTCATTTTACGCTTCACTGACTTTAAA | | |
| | CTTGATGGTACTGGTTATGGTGATTATGTCAAAATATATGATGGATTAGAGGAGAATCCA | | |
| | CACAAGCTTTTGCGTGTGTTCACAGCTTTTGATTCTCATGCACCTCTTACAGTTGTTTCT | | |
| | TCTTCTGGACAGATAAGGGTACATTTTTGTGCTGATAAAGTGAATGCTGCAAGGGGATTT | | |
| | AATGCTACTTACCAAGTAGATGGGTTCTGTTTGCCATGGGAAATACCCTGTGGAGGTGAC | | |
| | TGGGGGTGTTATACTGAGCAGCAGCGTTGTGATGGGTATTGGCATTGCCCAAATGGAAGG | | |
| | GATGAAACCAATTGTACCATGTGCCAGAAGGAAGAATTTCCATGTTCCCGAAATGGTGTC | | |
| | TGTTATCCTCGTTCTGATCGCTGCAACTACCAGAATCATTGCCCAAATGGCTCAGATGAA | | |
| | AAAAACTGCTTTTTTTGCCAACCAGCAAATTTCCATTGTAAAAACAATCGTTGTGTGTTT | | |
| | GAAAGTTGGGTGTGTGATTCTCAAGATGACTGTGGTGATGGCAGCGATGAAGAAAATTGC | | |
| | CCAGTAATCGTGCCTCGGCCG | | |
| | ORF Start: at 7 | | ORF Stop: at 1396 |
| | SEQ ID NO: 150 | 463 aa | MW at 52050.1kD |
| NOV31n, | NGALAEHSENVHISGVSTACGETPEQIRAPSGIITSPCWPSEYPAKINCSWFIRANPGEI | | |
| CG51264-14 | | | |
| Protein Sequence | ITTSFQDFDIQCSRRCNLDWLTIETYKNIESYPACGSTIPPPYTSSQDHIWIRFHSDDNI | | |
| | SRKGFRLAYFSGKSEEPNCACDQFRCGNGKCIPEAWKCNNMDECGDSSDEETCAKEANPP | | |
| | TAAAFQPCAYNQFQCLSRFTKVYTCLPESLKCDGNTDCLDPGDEIDCDVPTCGQWLKYFY | | |
| | GTFNSPNYPDFYPPGSNCTWLIDTCDHRKVILRFTDFKLDGTGYGDYVKTYDGLEENPHK | | |
| | LLRVLTAFDSHAPLTVVSSSGQIRVHFCADKVNAARGFNATYQVDGFCLPWEIPCGGDWG | | |
| | CYTEQQRCDGYWHCPNGRDETNCTMCQKEEFPCSRNGVCYPRSDRCNYQNHCPNGSDEKN | | |
| | CFFCQPGNFHCKNNRCVFESWVCDSQDDCGDGSDEENCPVIVP | | |
| | SEQ ID NO: 151 | 2592 bp | |
| NOV31o, | GGCCTGTCGCTCGAGCACAAAAGAGTCTCCCCGGTGCAGGTCTGCGTTGCTCTTCCTTTT | | |
| CG51264-15 | | | |
| DNA Sequence | CCTCGCTGGGGTGTACGGAAATGGTGCTCTTGCAGAACATTCTGAAAATGTGCATATTTC | | |
| | AGGAGTGTCAACTGCTTGTGGAGAGACTCCAGAGCAAATACGAGCACCAAGTGGCATAAT | | |
| | CACAAGCCCAGGCTGGCCTTCTGAATATCCTGCAAAAATCAACTGTAGCTGGTTCATAAG | | |
| | GGCAAACCCAGGCGAAATCATTACTATAAGTTTTCAGGATTTTGATATTCAAGGATCCAG | | |
| | TTGTGGTTCCACAATTCCACCTCCGTATATCTCTTCACAAGACCACATCTGGATTAGGTT | | |
| | TCATTCGGATGACAACATCTCTAGAAAGGGTTTCAGACTGGCATATTTTTCAGGGAAATC | | |
| | TGAGGAACCAAATTGTGCTTGTGATCAGTTTCGTTGTGGTAATGGAAAGTGTATACCAGA | | |
| | AGCCTGGAAATGTAATAACATGGATGAATGTGGAGATAGTTCCGATGAAGAGATCTGTGC | | |
| | CAAAGAAGCAAATCCTCCAACTGCTGCTGCTTTTCAACCCTGTGCTTACAACCAGTTCCA | | |
| | GTGTTTATCCCGTTTTACCAAAGTTTACACTTGCCTCCCCGAATCTTTAAAATGTGATGG | | |
| | GAACATTGACTGCCTTGACCTAGGAGATGAGATAGACTGTGATGTGCCAACATGTGGGCA | | |
| | ATGGCTAAAATATTTTTATGGTACTTTTAATTCTCCCAATTATCCAGACTTTTATCCTCC | | |
| | TGGAAGCAATTGCACCTGGTTAATAGACACTGGTGATCACCGTAAAGTCATTTTACGCTT | | |
| | CACTGACTTTAAACTTGATGGTACTGGTTATGGTGATTATGTCAAAATATATGATGGATT | | |
| | AGAGGAGAATCCACACAAGCTTTTGCGTGTGTTGACAGCTTTTGATTCTCATGCACCTCT | | |
| | TACAGTTGTTTCTTCTTCTGGACAGATAAGGGTACATTTTTGTGCTGATAAAGTGAATGC | | |
| | TGCAAGGGGATTTAATGCTACTTACCAAGTAGATGGGTTCTGTTTGCCATGGGAAATACC | | |
| | CTGTGGAGGTAACTGGGGGTGTTATACTGAGCAGCAGCGTTGTGATGGGTATTGGCATTG | | |
| | CCCAAATGGAAGGGATGAAACCAATTGTACCATGTGCCAGAAGGAAGAATTTCCATGTTC | | |
| | CCGAAATGGTGTCTGTTATCCTCGTTCTGATCGCTGCAACTACCAGAATCATTGCCCAAA | | |
| | TGGCTCAGATGAAAAAACTGCTTTTTTTTGCCAACCAGGAAATTTCCATTGTAAAAACAA | | |
| | TCGTTGTGTGTTTGAAAGTTGGGTGTGTGATTCTCAAGATGACTGTGGTGATGGCAGCGA | | |
| | TGAAGAAAATTGCCCAGTAATCGTGCCTACAAGAGTCATCACTGCTGCCGTCATAGGGAG | | |
| | CCTCATCTGCGGCCTGTTACTCGTCATAGCATTGGGATGTACTTGTAAGCTTTATTCTCT | | |
| | GAGAATGTTTGAAAGAAGATCATTTGAAACACAGTTGTCAAGAGTGGAAGCAGAATTGTT | | |
| | AAGAAGAGAAGCTCCTCCCTCGTATGGACAATTGATTGCTCAGGGTTTAATTCCACCAGT | | |
| | TGAAGATTTTCCTGTTTGTTCACCTAATCAGGCTTCTGTTTTGGAAAATCTGAGGCTAGC | | |
| | GGTACGATCTCAGCTTGGATTTACTTCAGTCAGGCTTCCTATGGCAGGCAGATCAAGCAA | | |
| | CATTTGGAACCGTATTTTTAATTTTGCAAGATCACGTCATTCTGGGTCATTGGCTTTGGT | | |
| | CTCAGCAGATGGAGATGAGGTTGTCCCTAGTCAGAGTACCAGTAGAGAACCTGAGAGAAA | | |
| | TCATACTCACAGAAGTTTGTTTTCCGTGGAGTCTGATGATACAGACACAGAAAATGAGAG | | |
| | AAGAGATATGGCAGGAGCATCTGGTGGGGTTGCAGCTCCTTTGCCTCAAAAAGTCCCTCC | | |
| | CACAACGGCAGTAGAAGCGACAGTAGGAGCATGTGCAAGTTCCTCAACTCAGAGTACCCG | | |
| | AGGTGGTCATGCAGATAATGGAAGGGATGTGACAAGTGTGGAACCCCCAAGTGTGAGTCC | | |
| | AGCACGTCACCAGCTTACAAGTGCACTCACTCGTATGACTCAQGCCCTACGCTGGGTACG | | |
| | TTTTACATTACGACGATCAAGTTCCCTAAGTCAGAACCACACTCCTTTGAGACAACTTGA | | |
| | TAATGGGGTAAGTGGAAGAGAAGATGATGATGATGTTGAAATGCTAATTCCAATTTCTGA | | |
| | TGGATCTTCAGACTTTCATGTGAATGACTGCTCCAGACCTCCTCTTCATCTTGCCTCAGA | | |
| | TCAAGGACAAGGGCTTAGACAACCATATAATGCAACAAATCCTGGAGTAAGGCCAAGTAA | | |
| | TCGAGATGGCCCCTGTGAGCGCTGTGGTATTGTCCACACTGCCCAGATACCAGACACTTG | | |
| | CTTAGAAGTAACACTGAAAAACGAAACGAGTGGTGATGAGGCTTTGTTACTTTGTTAGGT | | |
| | ACGAATCACATA | | |
| | ORF Start: at 2 | | ORF Stop: TAG at 2576 |
| | SEQ ID NO: 152 | 858 aa | MW at 94777.5kD |
| NOV31o, | ACRWSTKESPRWRSALLLLFLAGVYGNGALAEHSENVHISCVSTACGETPEQIRAPSGIT | | |
| CG51264-15 | | | |
| Protein Sequence | TSPGWPSEYPAKINCSWFIRANPGEIITISFQDFDIQGSRRCNLDWLTIETYKNIESYRA | | |
| | CGSTIPPPYISSQDHTWIRFHSDDNISRKGFRLAYFSGKSEEPNCACDQFRCGNCKCIPE | | |
| | AWKCNNMDECGDSSDEETCAKEANPPTAAAFQPCAYNQPQCLSRFTKVYTCLPESLKCDG | | |
| | NIDCLDLGDEIDCDVPTCGQWLKYPYGTFNSPNYPDFYPPCSNCTWLIDTGDHRKVILRF | | |
| | TDFKLDGTGYGDYVKIYDGLEENPNKLLRVLTAFDSHAPLTVVSSSGQIRVHPCADKVNA | | |
| | ARGFNATYQVDGFCLPWEIPCGGNWCCYTEQQRCDGYWHCPNCRDETNCTMCQKEEFPCS | | |
| | RNGVCYPRSDRCNYQNICPNGSDEKNCFFCQPCNFHCKNNRCVFESWVCDSQDDCGDGSD | | |
| | EENCPVIVPTRVITAAVIGSLICGLLLVIALGCTCKLYSLRMPERRSFETQLSRVEAELL | | |
| | RREAPPSYGQLIAQGLIPPVEDFPVCSPNQASVLENLRLAVRSQLGFTSVRLPMAGRSSN | | |
| | IWNRIFNFARSRHSGSLALVSADGDEVVPSQSTSREPERNHTHRSLPSVESDDTDTENER | | |
| | RDMAGASGGVAAPLPQKVPPTTAVEATVGACASSSTQSTRGGHADNGRDVTSVEPPSVSP | | |
| | ARHQLTSALSRMTQGLRWVRFTLGRSSSLSQNQSPLRQLDNGVSGREDDDDVEMLIPISD | | |
| | GSSDFDVNDCSRPPLDLASDQGQGLRQPYNATNPGVRPSNRDGPCERCGIVHTAQIPDTC | | |
| | LEVTLKNETSGDEALLLC | | |
| | SEQ ID NO: 153 | 2560 bp | |
| NOV31p, | TATGGCCTGTCGCTGGAGCACAAAAGAGTCTCCGCGGTCGAGGTCTGCGTTGCTCTTGCT | | |
| CG51264-16 | | | |
| DNA Sequence | TTTCCTCGCTGGGGTGTACGCTTGTGGAGAGACTCCAGGGCAAATACGAGCACCAAGTGG | | |
| | CATAATCACAAGCCCAGGCTGGCCTTCTGAATATCCTGCAAAAATCAACTCTAGCTGGTT | | |
| | CATAAGGCCAAACCCAGGCGAAATCATTACTATAAGTTTTCAGGATTTTGATATTCAAGG | | |
| | ATCCAGAAGGTGCAATTTGGACTGGTTGACAATAGAAACATACAAGAATATTGAAAGTTA | | |
| | CAGAGCTTGTGGTTCCACAATTCCACCTCCGTATATCTCTTCACAAGACCACATCTGGAT | | |
| | TAGGTTTCATTCGGATGACAACATCTCTAGAAAGGGTTTCAGACTGGCATATTTTTCAGG | | |
| | GAAATCTGAGGAACCAAATTGTGCTTGTGATCAGTTTCGTTGTGCTAATGGAAAGTGTAT | | |
| | ACCAGAAGCCTGGAAATGTAATAACATGGATCAATGTGGAGATAGTTCCGATGAAGAGAT | | |
| | CTGTGCCAAAGAAGCAAATCCTCCAACTGCTGCTGCTTTTCAACCCTGTGCTTACAACCA | | |
| | GTTCCAGTGTTTATCCCGTTTTACCAAAGTTTACACTTGCCTCCCCGAATCTTTAAAATG | | |
| | TGATGGGAACATTGACTGCCTTGACCTAGGAGATGAGATAGACTGTGATGTGCCAACATG | | |
| | TGGGCAATGCCTAAAATATTTTTATGGTACTTTTAATTCTCCCAATTATCCAGACTTTTA | | |
| | TCCTCCTGGAAGCAATTGCACCTGGTTAATAGACACTGGTGATCACCGTAAAGTCATTTT | | |
| | ACGCTTCACTGACTTTAAACTTGATGGTACTGCTTATGGTGATTATGTCAAAATATATGA | | |
| | TGGATTAGAGGAGAATCCACACAAGCTTTTGCGTGTGTTGACAGCTTTTGATTCTCATGC | | |
| | ACCTCTTACAGTTGTTTCTTCTTCTGGACAGATAAGGGTACATTTTTGTGCTGATAAAGT | | |
| | GAATGCTGCAAGGGGATTTAATGCTACTTACCAAGTAGATCGGTTCTGTTTGCCATGGGA | | |
| | AATACCCTGTGGAGGTAACTGGGGGTGTTATACTGAGCAGCAGCGTTGTGATGGGTATTG | | |
| | GCATTGCCCAAATGGAAGGGATGAAACCAATTGTACCATGTGCCAGAAGGAAGAATTTCC | | |
| | ATGTTCCCGAAATGGTGTCTGTTATCCTCGTTCTGATCGCTGCAACTACCAGAATCATTG | | |
| | CCCAAATGGCTCAGATGAAAAAAACTGCTTTTTTTGCCAACCAGGAAATTTCCATTCTAA | | |
| | AAACAATCGTTGTGTGTTTGAAAGTTGGGTGTGTGATTCTCAAGATGACTGTGGTGATGG | | |
| | CAGCGATCAAGAAAATTGCCCAGTAATCGTGCCTACAAGAGTCATCACTGCTGCCGTCAT | | |
| | AGGGAGCCTCATCTGTGGCCTGTTACTCGTCATAGCATTGGGATGTACTTGTAAGCTTTA | | |
| | TTCTCTGAGAATGTTTGAAAGAAGATCATTTGAAACACAGTTGTCAAGAGTGGAAGCAGA | | |
| | ATTGTTAAGAAGAGAAGCTCCTCCCTCGTATGGACAATTGATTGCTCAGGGTTTAATTCC | | |
| | ACCAGTTGAAGATTTTCCTGTTTGTTCACCTAATCAGGCTTCTGTTTTGGAAAATCTGAG | | |
| | GCTAGCGGTACGATCTCAGCTTGGATTTACTTCAGTCAGGCTTCCTATGGCACGCAGATC | | |
| | AAGCAACATTTGGAACCGTATTTTTAATTTTGCAAGATCACGTCATTCTGGGTCATTGGC | | |
| | TTTGGTCTCAGCAGATGGACATGAGGTTGTCCCTAGTCAGAGTACCAGTAGAGAACCTGA | | |
| | GAGAAATCATACTCACAGAAGTTTGTTTTCCGTGGAGTCTGATCATACACACACAGAAAA | | |
| | TGAGAGAAGAGATATCGCAGGAGCATCTCGTGGGGTTGCAGCTCCTTTGCCTCAAAAAGT | | |
| | CCCTCCCACAACGGCAGTAGAAGCGACAGTACGAGCATGTGCAAGTTCCTCAACTCACAG | | |
| | TACCCGAGGTGGTCATGCAGATAATGGAAGCGATGTGACAAGTGTGCAACCCCCAAGTGT | | |
| | GAGTCCAGCACGTCACCAGCTTACAAGTGCACTCAGTCGTATOACTCACGGGCTACGCTG | | |
| | GGTACGTTTTACATTAGGACGATCAAGTTCCCTAAGTCAGAACCAGAGTCCTTTGAGACA | | |
| | ACTTGATAATGGGGTAAGTGGAACAGAAGATGATGATGATGTTGAAATGCTAATTCCAAT | | |
| | TTCTGATGGATCTTCACACTTTGATGTGAATGACTGCTCCAGACCTCTTCTTGATCTTGC | | |
| | CTCAGATCAAGGACAAGGGCTTAGACAACCATATAATGCAACAAATCCTGCAGTAAGGCC | | |
| | AAGTAATCGAGATGGCCCCTGTGAGCGCTGTGGTATTGTCCACACTGCCCACATACCAGA | | |
| | CACTTGCTTACAAGTAACACTGAAAAACGAAACGAGTGATGATGAGGCTTTGTTACTTTG | | |
| | TTAGGTACGAATCACATAAGGGCGATTCCAGCACCTGGCT | | |
| | ORF Start: ATG at 2 | | ORF Stop: TAG at 2522 |
| | SEQ ID NO: 154 | 840 aa | MW at 93049.7kD |

| | |
|---|---|
| N0V31p, | MACRWSTKESPRWRSALLLLFLAGVYACGETPGQIRAPSCIITSPGWPSEYPAKINCSWF |
| CG51264-16 | |
| Protein Sequence | IRANPGEIITISFQDFDIQGSRRCNLDWLTIETYKNIESYRACGSTIPPPYISSQDHIWI |
| | RFHSDDNISRKGFRLAYFSGKSEEPNCACDQFRCGNGKCIPEAWKCNNMDECGDSSDEEI |
| | CAKEANPPTAAAPQPCAYNQFQCLSRFTKVYTCLPESLKCDGNIDCLDLGDEIDCDVPTC |
| | GQWLKYFYGTFNSPNYPDFYPPGSNCTWLIDTGDHRKVILRFTDFKLDCTGYGDYVKIYD |
| | GLEENPHKLLRVLTAFDSHAPLTVVSSSGQIRVHFCADKVNAARGFNATYQVDGFCLPWE |
| | IPCGGNWGCYTEQQRCDGYWHCPNGRDETNCTMCQKEEFPCSRNGVCYPRSDRCNYQNHC |
| | PNGSDEKNCFFCQPGNFHCKNNRCVFESWVCDSQDDCCDGSDEENCPVTVPTRVITAAVI |
| | GSLICGLLLVIALGCTCKLYSLRMFERRSFETQLSRVEAELLRREAPPSYGQLIAQGLIP |
| | PVEDFPVCSPNQASVLENLRLAVRSQLGFTSVRLPMAGRSSNIWNRIFNFARSRHSCSLA |
| | LVSADGDEVVPSQSTSREPERNHTNRSLFSVESDDTDTENERRDMAGASGGVAAPLPQKV |
| | PPTTAVEATVGACASSSTQSTRGGHADNGRDVTSVEPPSVSPARHQLTSALSRMTQGLRW |
| | VRFTLGRSSSLSQNQSPLRQLDIGVSGREDDDDVEMLIPISDCSSDFDVNDCSRPLLDLA |
| | SDQGQGLRQPYNATNPGVRPSNRDGPCERCGIVHTAQIPDTCLEVTLKNETSDDEALLLC |

Sequence comparison of the above protein sequences yields the following sequence relationships shown in Table 31B.

TABLE 31B
Comparison of NOV31a against NOV31b through NOV31p.

| | NOV31a Residues/ | Identities/Similarities |
|---|---|---|
| Protein Sequence | Match Residues | for the Matched Region |
| NOV31b | 1 . . . 423 | 422/442 (95%) |
| | 1 . . . 442 | 422/442 (95%) |
| NOV31c | 1 . . . 423 | 422/423 (99%) |
| | 1 . . . 423 | 422/423 (99%) |
| NOV31d | 1 . . . 840 | 826/840 (98%) |
| | 1 . . . 840 | 826/840 (98%) |
| NOV31e | 1 . . . 840 | 815/840 (97%) |
| | 1 . . . 837 | 816/840 (97%) |
| NOV31f | 1 . . . 423 | 422/423 (99%) |
| | 40 . . . 462 | 422/423 (99%) |
| NOV31g | 1 . . . 840 | 826/859 (96%) |
| | 1 . . . 859 | 826/859 (96%) |
| NOV31h | 27 . . . 471 | 430/445 (96%) |
| | 19 . . . 463 | 430/445 (96%) |
| NOV31i | 27 . . . 471 | 430/445 (96%) |
| | 19 . . . 463 | 430/445 (96%) |
| NOV31j | 28 . . . 471 | 429/444 (96%) |
| | 20 . . . 463 | 429/444 (96%) |
| NOV31k | 27 . . . 471 | 430/445 (96%) |
| | 19 . . . 463 | 430/445 (96%) |
| NOV31l | 27 . . . 471 | 431/445 (96%) |
| | 19 . . . 463 | 431/445 (96%) |
| NOV31m | 27 . . . 471 | 431/445 (96%) |
| | 19 . . . 463 | 431/445 (96%) |
| NOV31n | 27 . . . 471 | 429/445 (96%) |
| | 19 . . . 463 | 430/445 (96%) |
| NOV31o | 2 . . . 840 | 823/858 (95%) |
| | 1 . . . 858 | 823/858 (95%) |
| NOV31p | 1 . . . 840 | 825/840 (98%) |
| | 1 . . . 840 | 825/840 (98%) |

Further analysis of the NOV31a protein yielded the following properties shown in Table 31C.

TABLE 31C
Protein Sequence Properties NOV31a

| | |
|---|---|
| PSort analysis: | 0.4600 probability located in plasma membrane; |
| | 0.1000 probability located in endoplasmic reticulum |
| | (membrane); 0.1000 probability located in |
| | endoplasmic reticulum (lumen); 0.1000 probability |
| | located in outside |
| SignalP analysis: | Cleavage site between residues 28 and 29 |

A search of the NOV31a protein against the Geneseq database, a proprietary database that contains sequences published in patents and patent publication, yielded several homologous proteins shown in Table 31D.

TABLE 31D
Geneseq Results for NOV31a

| | | NOV31a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAB70544 | Human PRO14 protein | 1 . . . 840 | 840/840 (100%) | 0.0 |
| | sequence SEQ ID NO: 28 - | 1 . . . 840 | 840/840 (100%) | |
| | Homo sapiens, 840 aa. | | | |
| | [WO200110902-A2, 15- | | | |
| | FEB-2001] | | | |
| AAO20441 | Protein of the human cancer | 1 . . . 840 | 840/859 (97%) | 0.0 |
| | suppressor gene 98 - Homo | 36 . . . 894 | 840/859 (97%) | |
| | sapiens, 894 aa. | | | |
| | [CN1328030-A, 26-DEC- | | | |
| | 2001] | | | |
| AAU14316 | Human novel protein #187 - | 1 . . . 840 | 840/859 (97%) | 0.0 |
| | Homo sapiens, 859 aa. | 1 . . . 859 | 840/859 (97%) | |
| | [WO200155437-A2, 02- | | | |
| | AUG-2001] | | | |
| AAB42317 | Human ORFX ORF2081 | 1 . . . 840 | 840/859 (97%) | 0.0 |
| | polypeptide sequence SEQ | 1 . . . 859 | 840/859 (97%) | |
| | ID NO: 4162 - Homo sapiens, | | | |
| | 859 aa. [WO200058473-A2, | | | |
| | 05-OCT-2000] | | | |
| AAY02381 | Polypeptide identified by the | 1 . . . 840 | 840/859 (97%) | 0.0 |
| | signal sequence trap method - | 1 . . . 859 | 840/859 (97%) | |
| | Homo sapiens, 859 aa. | | | |
| | [WO9918126-A1, 15-APR- | | | |
| | 1999] | | | |

In a BLAST search of public sequence datbases, the NOV31a protein was found to have homology to the proteins shown in the BLASTP data in Table 31E.

TABLE 31E
Public BLASTP Results for NOV31a

| | | NOV31a | Identities/ | |
|---|---|---|---|---|
| Protein | | Residues/ | Similarities for | |
| Accession | | Match | the Matched | Expect |
| Number | Protein/Organism/Length | Residues | Portion | Value |
| CAC33422 | Sequence 27 from Patent | 1 . . . 840 | 840/840 (100%) | 0.0 |
| | WO0110902 - Homo sapiens | 1 . . . 840 | 840/840 (100%) | |
| | (Human), 840 aa. | | | |
| Q9Y561 | ST7 protein - Homo sapiens | 1 . . . 840 | 840/859 (97%) | 0.0 |
| | (Human), 859 aa. | 1 . . . 859 | 840/859 (97%) | |
| Q9BE74 | Hypothetical 73.8 kDa | 169 . . . 840 | 663/672 (98%) | 0.0 |
| | protein - Macaca fascicularis | 1 . . . 672 | 666/672 (98%) | |
| | (Crab eating macaque) | | | |
| | (Cynomolgus monkey), 672 | | | |
| | aa. | | | |
| CAC38967 | Sequence 19 from Patent | 1 . . . 423 | 422/423 (99%) | 0.0 |
| | WO0119856 - Homo sapiens | 1 . . . 423 | 422/423 (99%) | |
| | (Human), 430 aa. | | | |
| CAC33423 | Sequence 29 from Patent | 1 . . . 423 | 422/442 (95%) | 0.0 |
| | WO0110902 - Homo sapiens | 1 . . . 442 | 422/442 (95%) | |
| | (Human), 449 aa. | | | |

PFam analysis predicts that the NOV31a protein contains the domains shown in Table 31F.

TABLE 31F
Domain Analysis of NOV31a

| | | Identities/ | |
|---|---|---|---|
| | | Similarities | |
| | NOV31a | for the Matched | |
| Pfam Domain | Match Region | Region | Expect Value |
| CUB | 28 . . . 137 | 41/119 (34%) | 3.9e−31 |
| | | 89/119 (75%) | |
| Idl_recept_a | 145 . . . 183 | 19/43 (44%) | 2.1e−10 |
| | | 30/43 (70%) | |
| Idl_recept_a | 194 . . . 237 | 17/47 (36%) | 6.6e−05 |
| | | 27/47 (57%) | |
| CUB | 240 . . . 350 | 42/120 (35%) | 6.6e−23 |
| | | 83/120 (69%) | |
| Idl_recept_a | 354 . . . 393 | 15/43 (35%) | 0.072 |
| | | 23/43 (53%) | |
| Idl_recept_a | 394 . . . 431 | 17/44 (39%) | 0.045 |
| | | 27/44 (61%) | |
| Idl_recept_a | 432 . . . 468 | 21/43 (49%) | 1.4e−11 |
| | | 32/43 (74%) | |

### Example 32

The NOV32 clone was analyzed, and the nucleotide and encoded polypeptide sequences are shown in Table 32A.

TABLE 32A
NOV32 Sequence Analysis

| | SEQ ID NO: 155 | 2365 bp | |
|---|---|---|---|
| NOV32a, | ACGCGTTCGATATCCGCCCGGAGCTCCGGCGCAGCTCCTCCACCTTCGAGCTCATGAGAG | | |
| CG52423-01 | | | |
| DNA Sequence | CAGGCCTGGTGGTGAGCAGGGACGGTGCACCGGACGGCGGGATCGAGCAAATGGGTCTGG | | |
| | CCATGGAGCACGGAGGGTCCTACGCTCGGGCGGGGGGCAGCTCTCGGGGCTGCTGGTATT | | |
| | ACCTGCGCTACTTCTTCCTCTTCGTCTCCCTCATCCAATTCCTCATCATCCTGGGGCTCG | | |
| | TGCTCTTCATGGTCTATGGCAACGTGCACGTGAGCACAGAGTCCAACCTGCAGGCCACCG | | |
| | AGCGCCGAGCCGAGGGCCTATACAGTCAGCTCCTAGGGCTCACGGCCTCCCAGTCCAACT | | |
| | TGACCAAGGAGCTCAACTTCACCACCCGCGCCAAGGATGCCATCATGCAGATGTGGCTGA | | |
| | ATGCTCGCCGCGACCTGCACCGCATCAATGCCAGCTTCCGCCAGTGCCAGGGTGACCGGG | | |
| | TCATCTACACGAACAATCAGAGGTACATGGCTGCCATCATCTTGAGTGAGAAGCAATGCA | | |
| | GAGATCAATTCAAGGACATGAACAAGAGCTGCGATGCCTTGCTCTTCATGCTGAATCAGA | | |
| | AGGTGAAGACGCTGGAGGTGGAGATAGCCAAGGAGAAGACCATTTGCACTAAGGATAAGG | | |
| | AAAGCGTGCTGCTGAACAAACGCGTGGCGGAGGAACAGCTGGTTGAATGCGTGAAAACCC | | |
| | GGGAGCTGCAGCACCAAGAGCGCCAGCTGGCCAAGGAGCAACTGCAAAAGGTGCAAGCCC | | |
| | TCTGCCTGCCCCTGGACAAGGACAAGTTTGAGATGGACCTTCGTAACCTGTGGAGGGACT | | |
| | CCATTATCCCACGCAGCCTGGACAACCTGGGTTACAACCTCTACCATCCCCTGGGCTCGG | | |
| | AATTGGCCTCCATCCGCAGAGCCTGCGACCACATGCCCAGCCTCATGAGCTCCAAGGTGG | | |
| | AGGACCTGGCCCGCAGCCTCCGGGCGCATATCGAACGCGTCGCCCGCGAGAACTCAGACC | | |
| | TCCAACGCCAGAAGCTGGAAGCCCAGCAGGGCCTGCGGGCCAGTCAGGAGGCGAAACAGA | | |
| | AGGTGGAGAAGGAGGCTCAGGCCCGGGACCCCAAGCTCCAAGCTGAATGCTCCCGGCAGA | | |
| | CCCAGCTAGCGCTGGAGGAGAAGGCGGTGCTGCGGAAGGAACGAGACAACCTGGCCAAGG | | |
| | AGCTGGAAGAGAAGAAGAGGGAGGCGGAGCAGCTCAGGATGGAGCTGGCCATCAGAAACT | | |
| | CCCAGCTAGCGCTGGAGGAGAAGGCGGTGCTGCGGAAGGAACGAGACAACCTGGCCAAGG | | |
| | AGCTGGAAGAGAAGAAGAGGGAGGCGGAGCAGCTCAGGATGGAGCTGGCCATCAGAAACT | | |
| | AGATCCTGGAGTCCCAGAGCCCCCCTCCAGGCATCCCTGTAGCCCCATCCAGTGCCTGAG | | |
| | GAGGCTCCAGGCCTCAGGACCAAGGCATGGCCCGACTCGGCGGTTTGCGCAGGATGCAGG | | |
| | GATATCCTCACACCGCCCGACACAACCCCCTCCCGCCGCCCCCAACCACCCAGGGCCACC | | |
| | ATCAGACAACTCCCTGCATGCAAACCCCTAGTACCCTCTCACACCCGCACCCGCGCCTCA | | |
| | CGATCCCTCACCCAGAGCACACGGCCGCGGAGATGACGTCACCCAAGCAACGGCGCTGAC | | |
| | GTCACATATCACCGTGGTGATGGCGTCACGTCGCCATGTAGACGTCACGAAGAGATATAC | | |
| | CGATGGCGTCGTGCAGATGCAGCACGTCGCACACACACATGGCGAACTTGGCATGACGTC | | |
| | ACACCGAGATGCAGCAACGACGTCACGGGCCATGTCGACGTCACACATATTAATGTCACA | | |
| | CAGACGCGGCGATGGCATCACACAGACGCTCATGATGTCACACACAGACACAGTGACAAC | | |
| | ACACACCATGACAACGACACCTATAGATATGGCACCAACATCACATGCACGCATGCCCTT | | |
| | TCACACACACTTTCTACCCAATTCTCACCTAGTCTCACGTTCCCCCGACCCTGGCACACG | | |
| | GGCCAAGGTACCCACACGATCCCATCCCCTCCCGCACACCCCTCGCCCCCAGCACCTCCC | | |
| | CTCCTCCAGCTTCCTGGCCTCCCAGCCACTTCCTCACCCCCAGTGCCTGGACCCGGACGT | | |
| | GAGAACAGGAACCCATTCACCTCCGCTCCTTGACCGTGAGTGTTTCCACGACCCCCTCGG | | |
| | GGCCCTCAGCCGGGGGTGAGGGTCACCTGTTGTCGGGAGGCGAGCCACTCCTTCTCCCCC | | |
| | AACTCCCAGCCCTGCCTGTGGCCCGTTGAAATGTTGGTGCCACTTAATAAATATTAGTAA | | |
| | ATCCTTAAAAAAAAAAAAAAAAAA | | |
| | ORF Start: ATG at 54 | | ORF Stop: TGA at 1437 |
| | SEQ ID NO: 156 | 461 aa | MW at 52503.8kD |

| | |
|---|---|
| NOV32a, | MRAGLVVSRDGAPDGGIEQMGLAMEHGGSYARAGGSSRGCWYYLRYFFLFVSLIQFLIIL |
| CG52423-01 | |
| Protein Sequence | GLVLFMVYGNVHVSTESNLQATERRAEGLYSQLLGLTASQSNLTKELNFTTRAKDAIMQM |
| | WLNARRDLDRINASFRQCQGDRVIYTNNQRYMAAIILSEKQCRDQFKDMNKSCDALLFML |
| | NQKVKTLEVEIAKEKTICTKDKESVLLNKRVAEEQLVECVKTRELQHQERQLAKEQLQKV |
| | QALCLPLDKDKFEMDLRNLWRDSIIPRSLDNLGYNLYHPLCSELASIRRACDHMPSLMSS |
| | KVEELARSLRADIERVARENSDLQRQKLEAQQGLRASQEAKQKVEKEAQAREAKLQAECS |
| | RQTQLALEEKAVLRKERDNLAKELEEKKREAEQLRMELAIRNSALDTCIKTKSQPMMPVS |
| | RPMGPVPNPQPIDPASLEEFKRKILESQRPPAGIPVAPSSG |

Twenty polymorphic variants of NOV32a have been identified and are shown in Table 41L. Further analysis of the NOV32a protein yielded the following properties shown in Table 32B.

TABLE 32B
Protein Sequence Properties NOV32a

| | |
|---|---|
| PSort | 0.7900 probability located in plasma membrane; 0.6000 |
| analysis: | probability located in nucleus; 0.3000 probability located in |
| | microbody (peroxisome); 0.3000 probability located in |
| | Golgi body |
| SignalP | Cleavage site between residues 70 and 71 |
| analysis: | |

A search of the NOV32a protein against the Geneseq database, a proprietary database that contains sequences published in patents and patent publication, yielded several homologous proteins shown in Table 32C.

TABLE 32C
Geneseq Results for NOV32a

| | | NOV32a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAB42154 | Human ORFX ORF1918 | 1 . . . 461 | 461/461 (100%) | 0.0 |
| | polypeptide sequence SEQ | 19 . . . 479 | 461/461 (100%) | |
| | ID NO: 3836 - Homo sapiens, | | | |
| | 479 aa. [WO200058473-A2, | | | |
| | 05-OCT-2000] | | | |
| AAM41619 | Human polypeptide SEQ ID | 7 . . . 461 | 454/455 (99%) | 0.0 |
| | NO 6550 - Homo sapiens, | 3 . . . 457 | 454/455 (99%) | |
| | 457 aa. [WO200153312-A1, | | | |
| | 26-JUL-2001] | | | |
| AAE06600 | Human protein having | 20 . . . 461 | 442/442 (100%) | 0.0 |
| | hydrophobic domain, | 1 . . . 442 | 442/442 (100%) | |
| | HP10787 - Homo sapiens, | | | |
| | 442 aa. [WO200149728-A2, | | | |
| | 12-JUL-2001] | | | |
| AAM39833 | Human polypeptide SEQ ID | 20 . . . 461 | 439/442 (99%) | 0.0 |
| | NO 2978 - Homo sapiens, | 1 . . . 442 | 439/442 (99%) | |
| | 442 aa. [WO200153312-A1, | | | |
| | 26-JUL-2001] | | | |
| AAY12280 | Human 5′ EST secreted | 20 . . . 124 | 104/105 (99%) | 3e−54 |
| | protein SEQ ID NO: 311 - | 1 . . . 105 | 104/105 (99%) | |
| | Homo sapiens, 105 aa. | | | |
| | [WO9906548-A2, 11-FEB- | | | |
| | 1999] | | | |

In a BLAST search of public sequence datbases, the NOV32a protein was found to have homology to the proteins shown in the BLASTP data in Table 32D.

TABLE 32D
Public BLASTP Results for NOV32a

| | | NOV32a | | |
|---|---|---|---|---|
| Protein | | Residues/ | Identities/ | |
| Accession | | Match | Similarities for the | Expect |
| Number | Protein/Organism/Length | Residues | Matched Portion | Value |
| CAD39027 | Hypothetical protein - Homo | 6 . . . 461 | 456/456 (100%) | 0.0 |
| | sapiens (Human), 456 aa | 1 . . . 456 | 456/456 (100%) | |
| | (fragment). | | | |
| Q9BX97 | PV1 protein - Homo sapiens | 20 . . . 461 | 442/442 (100%) | 0.0 |
| | (Human), 442 aa. | 1 . . . 442 | 442/442 (100%) | |
| Q9BZD5 | Fenestrated-endothelial | 20 . . . 461 | 441/442 (99%) | 0.0 |
| | linked structure protein - | 1 . . . 442 | 441/442 (99%) | |
| | Homo sapiens (Human), 442 | | | |
| | aa. | | | |
| BAC04681 | CDNA FLJ38711 fis, clone | 20 . . . 461 | 436/442 (98%) | 0.0 |
| | KIDNE2003507, highly | 1 . . . 437 | 436/442 (98%) | |
| | similar to Homo sapiens PV1 | | | |
| | protein (PLVAP) mRNA - | | | |
| | Homo sapiens (Human), 437 | | | |
| | aa. | | | |
| Q91VC4 | MECA32 (Similar to | 20 . . . 461 | 273/442 (61%) | e−156 |
| | PLASMALEMMA vesicle | 1 . . . 438 | 351/442 (78%) | |
| | associated protein) - Mus | | | |
| | musculus (Mouse), 438 aa. | | | |

PFam analysis predicts that the NOV32a protein contains the domains shown in Table 32E.

TABLE 32E
Domain Analysis of NOV32a

| | | | |
|---|---|---|---|
| Pfam Domain | NOV32a Match | Identities/ | Expect Value |
| | Region | Similarities | |
| | | for the Matched | |
| | | Region | |

### Example 33

The NOV33 clone was analyzed, and the nucleotide and encoded polypeptide sequences are shown in Table 33A.

TABLE 33A
NOV33 Sequence Analysis

| | |
|---|---|
| SEQ ID NO: 157 | 1482 bp |

| NOV33a, | CCAGGCGCTGGCCGTGGTGCTGATTCTGTCAGGCGCTGGCGGCGGCAGCCGGCGTGACGG | |
|---|---|---|
| CG52919-01 | | |
| DNA Sequence | CTGCGGCCCCGCTCCCTCTACCCGGCCCGACCCGGCTCTGCCCCCGCGCCCAAGCCCCAC | |
| | CAAGCCCCCCGCCCTCCCGCCGCGGTCCCAGCCCAGGGCGCGGCCGCAACCAGCACCATG | |
| | CGCCCGGTAGCCCTGCTGCTCCTGCCCTCGCTGCTGGCGCTCCTGGCTCACGGACTCTCT | |
| | TTAGACCCCCCAACCGTGCCGAAAGGACAAGCCCCAGGCATCGAGGAGACAGATGGCGAG | |
| | CTGACACCAGCCCCCACACCTGAGCACCCAGAACGAGCCGTCCACTTTGTCACAACAGCC | |
| | CCCACCTTGAAGCTGCTCAACCACCACCCGCTGCTTGAGGAATTCCTACAAGAGGGGCTG | |
| | GAAAAGGGAGATGAGGAGCTGAGGCCAGCACTGCCCTTCCAGCCTGACCCACCTGCACCC | |
| | TTCACCCCAAGTCCCCTTCCCCGCCTGGCCAACCAGGACAGCCGCCCTGTCTTTACCAGC | |
| | CCCACTCCAGCCATGGCTGCGGTACCCACTCAGCCCCAGTCCAAGGAGGGACCCTGGAGT | |
| | CCGGAGTCAGAGTCCCCTATGCTTCGAATCACAGCTCCCCTACCTCCAGGGCCCAGCATG | |
| | GCAGTGCCCACCCTAGGCCCAGGGGAGATAGCCAGCACTACACCCCCCAGCAGAGCCTGG | |
| | ACACCAACCCAAGAGGGTCCTGGACACATGCGAAGGCCGTGGGTTGCAGAGGTTGTGTCC | |
| | CAGCGCCCAGGCATCGCGATCCAGGGGACCATCACCTCCTCCACACCTTCAGGAGATGAT | |
| | GAGGAGACCACCACTACCACCACCATCATCACCACCACCATCACCACAGTCCAGACACCA | |
| | GGTCAGCTACCTGCTGGCTTGCAGATGTCCAAATGGGGATGGGCGACCCTGCCGGGCCCC | |
| | TAAAAGCCTGTCTCTGACACTGTGCCAGCCTGCCCTGCCCTTTGGCACCAAGGGCCAGCC | |
| | TGCAGGAGGCATGTAGATTGGACCCAGATAGACCTGAGCTCAAATCCTGATTCTTCAGCC | |
| | AAGTACAGTGGCTCATGCCTGTAATCCCAGCACTTTGGGAGGCAGAGGCCAGTGGATCAT | |
| | CTGAGGTCAGGAGTTCAAGACCCTCCTGGCCAACATGGCGAAACACCATCTCTACTAAAA | |
| | ATACAAAAATGAGCCGGGCATGGTGGTGGGCACCTGTAATCCCAGCTACTCGGGAGGCTG | |
| | AGGCAGGAGAATCACTCAAACCTGGGAGGCAGAGGTTGCAGTGAGCTGAGATTGCACCAT | |
| | TGCACTCCAGCCTGGGCAACAGAGCGAGACTCTGTCTCAAAAAAGAAAAAATCTTGATTC | |
| | TTCCAACTATAACATGACCCTAGGAATTCTATTTAACATCTCATCTCTGAGCCTCATCTG | |
| | TAAAATGGCAATAAGAAAATAAACTTCTCGCTAGAAAAAAAA | |
| | ORF Start: ATG at 178 | ORF Stop: 27471.8kD |

| | | |
|---|---|---|
| SEQ ID NO: 158 | 261 aa | MW at 27471.8kD |

| NOV33a, | MRPVALLLLPSLLALLAHGLSLEAPTVGKGQAPGIEETDGELTAAPTPEQPERCVHFVTT | |
|---|---|---|
| CG52919-01 | | |
| Protein Sequence | APTLKLLNHHPLLEEFLQEGLEKCDEELRPALPFQPDPPAPFTPSPLPRLANQDSRPVFT | |
| | SPTPAMAAVPTQPQSKEGPWSPESESPMLRITAPLPPGPSMAVPTLGPGEIASTTPPSRA | |
| | WTPTQEGPGDMGRPWVAEVVSQGAGIGIQGTITSSTASCDDEETTTTTTIITTTTTTVQT | |
| | PGQLPAGLQMWKWGWGRLRGP | |
| | SEQ ID NO: 159 | 2127 bp |

| NOV33b, | CCAGGCGCTCGCCGTGGTGCTCATTCTGTCAGGCGCTGGCGGCGGCAGCCGCGGTGACGG | |
|---|---|---|
| CG52919-02 | | |
| DNA Sequence | CTGCGGCCCCGCTCCCTCTACCCGCCCGGACCCGGCTCTGCCCCCGCCCCCAAGCCCCAC | |
| | CAAGCCCCCCGCCCTCCCGCCGCGGTCCCAGCCCAGGGCGCGGCCGCAACCAGCACCATG | |
| | CGCCCGGTAGCCCTGCTCCTCCTGCCCTCGCTGCTGGCGCTCCTGGCTCACGGACTCTCT | |
| | TTAGAGGCCCCAACCGTGGCGAAAGGACAACCCCCACGCATCGAGGAGACAGATGGCGAG | |
| | CTGACAGCAGCCCCCACACCTGAGCAGCCAGAACGAGGCGTCCACTTTGTCACAACAGCC | |
| | CCCACCTTGAAGCTGCTCAACCACCACCCCCTGCTTGAGGAATTCCTACAAGAGGGGCTG | |
| | GAAAACGGACATGAGGAGCTGAGGCCAGCACTGCCCTTCCAGCCTGACCCACCTGCACCC | |
| | TTCACCCCAAGTCCCCTTCCCCCCCTCGCCAACCAGGACAGCCCCCCTGTCTTTACCAGC | |
| | CCCACTCCAGCCATGCCTGCGGTACCCACTCACCCCCAGTCCAAGGAGGGACCCTGGAGT | |
| | CCGGACTCAGAGTCCCCTATGCTTCCAATCACACCTCCCCTACCTCCAGGGCCCAGCATG | |
| | GCAGTGCCCACCCTAGGCCCAGGGGAGATACCCAGCACTACACCCCCCAGCACAGCCTCG | |
| | ACACCAACCCAACAGGGTCCTGGAGACATGGGAAGGCCGTGGGTTGCAGACGTTGTGTCC | |
| | CAGGGCGCACGGATCCGGATCCACGGGACCATCACCTCCTCCACAGCTTCAGGAGATGAT | |
| | GAGGAGACCACCACTACCACCACCATCATCACCACCACCATCACCACAGTCCAGACACCA | |
| | GGCCCTTGTACCTGGAATTTCTCAGGCCCACAGGGCTCTCTGGACTCCCCTACAGACCTC | |
| | AGCTCCCCCACTGATGTTGGCCTCGACTGCTTCTTCTACATCTCTGTCTACCCTGGCTAT | |
| | GGCGTCCAAATCAAGCTCCAGAATATCAGCCTCCGGGAAGGGGAGACAGTGACTGTGCAA | |
| | GGCCTCGCGCGGCCTGACCCACTGCCCCTGGCCAACCAGTCTTTCCTGCTGCGGGGCCAA | |
| | GTCATCCCCAGCCCCACCCACCAACCCGCCCTGAGGTTCCAGAGCCTCCCGCCACCGGCT | |
| | GGCCCTGGCACCTTCCATTTCCATTACCAAGCCTATCTCCTGAGCTGCCACTTTCCCCGT | |
| | CGTCCAGCTTATGGAGATGTGACTGTCACCAGCCTCCACCCAGGGGGTAGTGCCCGCTTC | |
| | CATTGTCCCACTCCCTACCACCTGAAGGGCGCCACGCATCTCACCTGTCTCAATGCCACC | |
| | CAGCCCTTCTGCGATTCAAAGGAGCCCGTCTCCATCGCTGCTTGCGGCGGAGTGATCCGC | |
| | AATGGCACCACCGGCCGCATCGTCTCTCCACGCTTCCCGGGCAACTACAGCAACAACCTC | |
| | ACCTGTCACTGGCTGCTTGAGGCTCCTGAGGGCCAGCGGCTACACCTGCACTTTGACAAG | |
| | GTTTCCCTCCCAGAGCATGATGACAGGCTCATCATTCGCAATGGGGACAACGTGGAGGCC | |
| | CCACCAGTGCGAAAAAGCTCCCTGCAGCTGCCCCGCCCCCGCCCCCGCCCCTACAACCGC | |
| | ATTACCATAGAGTCAGCGTTTGACAATCCAACTTACGAGACTGGAGAGACGAGAGAATAT | |
| | GAAGTCTCCATCTAGGTGGGGGCAGTCTAGGGAAGTCAACTCAGACTTGCACCACAGTCC | |
| | AGCAGCAAGGCTCCTTGCTTCCTGCTCTCCCTCCACCTCCTGTATATACCACCTAGGAGG | |
| | AGATGCCACCAAGCCCTCAAGAAGTTGTGCCCTTCCCCGCCTGCGATGCCCACCATGGCC | |
| | TATTTTCTTGGTGTCATTCCCCACTTGGGGCCCTTGCATTGGGCCATGTACAGGGGGCAT | |
| | CTACCTGTGGGGAAGAACATAGCTGGGAGCACAAGCTTCAACAGCCAGCATTCCTTGAGC | |
| | CTCCTTCATGCCCCTGGGACCAGCCTGGGGAACACANTTACGCAGGAGCAGGGAGTTACC | |
| | TTGTTTCACATGACCACCAACCATTCC | |
| | ORF Start: ATG at 178 | ORF Stop: TAG at 1753 |

| | | |
|---|---|---|
| SEQ ID NO: 160 | 525 aa | MW at 56283.7kD |

| NOV33b, | MRPVALLLLPSLLALLAHGLSLEAPTVGKGQAPGIEETDGELTAAPTPEQPERGVHFVTT | |
|---|---|---|
| CG52919-02 | | |
| Protein Sequence | APTLKLLNHHPLLEEFLQEGLEKGDEELRPALPFQPDPPAPFTPSPLPRLANQDSRPVFT | |
| | SPTPAMAAVPTQPQSKEGPWSPESESPMLRITAPLPPGPSMAVPTLGPGEIASTTPPSRA | |
| | WTPTQEGPGDMGRPWVAEVVSQGAGIGIQGTITSSTASGDDEETTTTTTIITTTITTVQT | |
| | PGPCSWNFSGPEGSLDSPTDLSSPTDVGLDCFFYISVYPGYGVEIKVQNISLREGETVTV | |
| | EGLGGPDPLPLANQSFLLRGQVIRSPTHQAALRFQSLPPPAGPGTFHFHYQAYLLSCHFP | |
| | RRPAYGDVTVTSLXPGGSARFHCATGYQLKGARHLTCLNATQPFWDSKEPVCIAACGGVI | |
| | RNGTTGRIVSPGFPGNYSNNLTCHWLLEAPEGQRLHLHFEKVSLAEDDDRLIIRNGDNVE | |
| | APPVGKSSLQLPRPRPRPYNRITIESAFDNPTYETGETREYEVSI | |
| | SEQ ID NO: 161 | 2127 bp |

| NOV33c, | CCAGGCGCTGGCCGTGGTCCTGATTCTGTCAGGCGCTGCCGGCGGCAGCGGCGGTCACGG | |
|---|---|---|
| CG52919-03 | | |
| DNA Sequence | CTGCGCCCCCGCTCCCTCTACCCGGCCGGACCCGGCTCTGCCCCCGCGCCCAAGCCCCAC | |
| | CAAGCCCCCCGCCCTCCCGCCGCGGTCCCAGCCCAGGGCGCGGCCGCAACCAGCACCATG | |
| | CGCCCCCTAGCCCTCCTGCTCCTGCCCTCGCTGCTGGCGCTCCTGGCTCACGGACTCTCT | |
| | TTAGAGGCCCCAACCGTGGCGAAAGGACAAGCCCCAGGCATCCAGGACACAGATCGCGAG | |
| | CTGACAGCAGCCCCCACACCTGAGCAGCCAGAACGAGGCGTCCACTTTGTCACAACAGCC | |
| | CCCACCTTGAAGCTGCTCAACCACCACCCGCTGCTTGAGCAATTCCTACAAGACCGGCTG | |
| | GAAAAGGGAGATGAGGAGCTGACGCCAGCACTGCCCTTCCAGCCTGACCCACCTGCACCC | |
| | TTCACCCCAAGTCCCCTTCCCCGCCTGGCCAACCACCACAGCCGCCCTGTCTTTACCAGC | |
| | CCCACTCCAGCCATGGCTGCGGTACCCACTCAGCCCCAGTCCAAGCACGGACCCTGGAGT | |
| | CCGGACTCAGAGTCCCCTATGCTTCGAATCACAGCTCCCCTACCTCCAGCGCCCAGCATG | |
| | GCAGTGCCCACCCTAGGCCCAGCCGACATAGCCAGCACTACACCCCCCAGCAGAGCCTCG | |
| | ACACCAACCCAAGAGCGTCCTGGAGACATGGGAAGGCCGTGGGTTCCACAGGTTGTGTCC | |
| | CAGGGCCCAGGGATCCCGATCCAGGGGACCATCACCTCCTCCACAGCTTCAGCAGATGAT | |
| | GAGGAGACCACCACTACCACCACCATCATCACCACCACCATCACCACAGTCCAGACACCA | |
| | GGCCCTTGTACCTGGAATTTCTCAGGCCCAGAGGGCTCTCTGGACTCCCCTACAGACCTC | |
| | AGCTCCCCCACTGATGTTCCCCTGGACTGCTTCTTCTACATCTCTGTCTACCCTCGCTAT | |
| | GGCGTGGAAATCAAGGTCCAGAATATCAGCCTCCCGCAAGGCGAGACAGTGACTCTGGAA | |
| | GGCCTCGGGGGGCCTGACCCACTGCCCCTGGCCAACCAGTCTTTCCTCCTGCGCCCCCAA | |
| | GTCATCCGCAGCCCCACCCACCAAGCGGCCCTGAGGTTCCACACCCTCCCGCCACCGGCT | |
| | GGCCCTGGCACCTTCCATTTCCATTACCAACCCTATCTCCTGAGCTGCCACTTTCCCCGT | |
| | CGTCCACCTTATGGAGATGTGACTGTCACCACCCTCCACCCAGGGGGTAGTGCCCGCTTC | |
| | CATTGTCCCACTGCCTACCAGCTGAAGCGCGCCAGGCATCTCACCTGTCTCAATCCCACC | |
| | CAGCCCTTCTCGCATTCAAAGGAGCCCGTCTGCATCGCTGCTTCCGGCGCAGTGATCCGC | |
| | AATGGCACCACCGGCCGCATCGTCTCTCCAGGCTTCCCGGGCAACTACACCAACAACCTC | |
| | ACCTGTCACTGGCTGCTTGACGCTCCTGAGGGCCAGCGGCTACACCTGCACTTTGAGAAG | |
| | GTTTCCCTGGCAGAGGATGATGACACGCTCATCATTCGCAATGGCCACAACGTGGAGGCC | |
| | CCACCAGTGTATGATTCCTATGAGGTGGAATACCCGCCCCCCCCCCGCCCCTACAACCGC | |
| | ATTACCATAGAGTCAGCGTTTGACAATCCAACTTACGAGACTGGAGAGACGAGAGAATAT | |
| | GAAGTCTCCATCTAGGTGGGGGCAGTCTAGGGAAGTCAACTCAGACTTGCACCACAGTCC | |
| | AGCAGCAAGCCTCCTTGCTTCCTGCTGTCCCTCCACCTCCTGTATATACCACCTAGGAGG | |
| | AGATGCCACCAACCCCTCAAGAACTTGTGCCCTTCCCCGCCTGCGATGCCCACCATGGCC | |
| | TATTTTCTTGGTGTCATTGCCCACTTGGGGCCCTTGCATTGGGCCATGTACACGGGGCAT | |
| | CTACCTGTGGGGAAGAACATAGCTGGGAGCACAAGCTTCAACACCCAGCATTCCTTGAGC | |
| | CTCCTTCATGGCCCTGGGACCAGCCTGGGGAACACANTTAGGCAGCACCAGCGACTTACC | |
| | TTGTTTCACATGACCACCAACCATTCC | |
| | ORF Start: ATG at 178 | ORF Stop: TAG at 1753 |

| | | |
|---|---|---|
| SEQ ID NO: 162 | 525 aa | MW at 56462.7kD |

| NOV33c, | MRPVALLLLPSLLALLAHGLSLEAPTVGKGQAPGIEETDGELTAAPTPEQPERGVHFVTT | |
|---|---|---|
| CG52919-03 | | |
| Protein Sequence | APTLKLLNHHPLLEEFLQEGLEKGDEELRPALPFQPDPPAPFTPSPLPRLANQDSRPVFT | |
| | SPTPAMAAVPTQPQSKEGPWSPESESPMLRITAPLPPGPSMAVPTLGPGEIASTTPPSRA | |
| | WTPTQEGPGDMGRPWVAEVVSQGAGIGIQGTITSSTASGDDEETTTTTTIITTTITTVQT | |
| | PGPCSWNFSGPEGSLDSPTDLSSPTDVGLDCFFYISVYPGYGVEIKVQNISLREGETVTV | |
| | EGLGGPDPLPLANQSFLLRGQVIRSPTHQAALRFQSLPPPAGPGTFHFHYQAYLLSCHFP | |
| | RRPAYGDVTVTSLNPGGSARFHCATGYQLKGARHLTCLNATQPFWDSKEPVCIAACGGVI | |
| | RNGTTCRIVSPGFPGNYSNNLTCHWLLEAPEGQRLHLHFEKVSLAEDDDRLIIRNGDNVE | |
| | APPVYDSYEVEYPPRPRPYNRITIESAFDNPTYETGETREYEVSI | |
| | SEQ ID NO: 163 | 1988 bp |

| NOV33d, | CCAGGCGCTGGCCGTGGTGCTGATTCTGTCAGGCGCTGGCGGCGGCAGCGGCGGTCACGG | |
|---|---|---|
| CG52919-04 | | |
| DNA Sequence | CTGCGGCCCCGCTCCCTCTACCCGGCCGGACCCGCCTCTGCCCCCGCGCCCAAGCCCCAC | |
| | CAAGCCCCCCGCCCTCCCGCCGCGGTCCCAGCCCAGGGCGCGGCCGCAACCAGCACCATG | |
| | CGCCCCGTACCCCTGCTGCTCCTGCCCTCCCTGCTGGCGCTCCTGGCTCACGGACTCTCT | |
| | TTAGAGGCCCCAACCGTGGGCAAACGACAAGCCCCAGGCATCGAGCAGACAGATCGCGAG | |
| | CTGACAGCAGCCCCCACACCTGAGCAGCCAGAACGAGGCGTCCACTTTCTCACAACAGCC | |
| | CCCACCTTGAAGCTGCTCAACCACCACCCGCTGCTTGAGGAATTCCTACAACAGGGGCTG | |
| | GAAAAGGGAGATGAGGAGCTGAGGCCAGCACTGCCCTTCCACCCTGACCCACCTCCACCC | |
| | TTCACCCCAACTCCCCTTCCCCGCCTGCCCAACCACGACAGCCGCCCTGTCTTTACCAGC | |
| | CCCACTCCAGCCATGGCTCCGGTACCCACTCAGCCCCAGTCCAAGGACCCACCCTCGACT | |
| | CCGGAGTCAGACTCCCCTATCCTTCGAATCACAGCTCCCCTACCTCCAGGGCCCACCATG | |
| | GCAGTGCCCACCCTAGGCCCAGCGGAGATAGCCAGCACTACACCCCCCAGCAGACCCTGG | |
| | ACACCAACCCAAGAGCGTCCTGGACACATGGGAAGGCCGTGGGTTGCACAGGTTGTCTCC | |
| | CAGGCCGCAGGGATCGGGATCCAGGGGACCATCACCTCCTCCACAGCTTCAGGAGATGAT | |
| | GAGGAGACCACCACTACCACCACCATCATCACCACCACCATCACCACACTCCAGACACCA | |
| | GGCCCTTGTAGCTGGAATTTCTCAGGCCCAGAGGGCTCTCTGGACTCCCCTACAGACCTC | |
| | AGCTCCCCCACTGATGTTGGCCTGGACTGCTTCTTCTACATCTCTGTCTACCCTGGCTAT | |
| | GGCGTCGAAATCAAGGTCCAGAATATCAGCCTCCGGGAAGGCCAGACAGTGACTGTGGAA | |
| | GGCCTCGGCGGGCCTGACCCACTGCCCCTCCCCAACCAGTCTTTCCTGCTCCGGGCCCAA | |
| | GTCATCCGCAGCCCCACCCACCAAGCGGCCCTGAGGTTCCAGACCCTCCCGCCACCGGCT | |
| | GGCCCTGCCACCTTCCATTTCCATTACCAAGCCTATCTCCTCAGCTGCCACTTTCCCCGT | |
| | CGTCCAGCTTATGGAGATGTGACTGTCACCACCCTCCACCCAGGCGGTAGTGCCCCCTTC | |
| | CATTGTGCCACTCCCTACCAGCTGAAGCGCGCCAGGCATCTCACCTGTCTCAATGCCACC | |
| | CAGCCCTTCTGGGATTCAAACGAGCCCGTCTGCATCGCTGCTTCCGGCGGAGTGATCCGC | |
| | AATGGCACCACCGCCCGCATCGTCTCTCCAGGCTTCCCCCGCAACTACAGCAACAACCTC | |
| | ACCTGTCACTGGCTGCTTGAGGCTCCTGAGGGCCAGCCCCTACACCTGCACTTTGAGAAG | |
| | GTTTCCCTGGCAGAGGATGATGACAGGCTCATCATTCGCAATGGGGACAACGTGGAGCCC | |
| | CCACCAGTGTATGATTCCTATGAGGTGGAATACCCGCCCCGCCCCCGCCCCTACAACCGC | |
| | ATTACCATAGAGTCAGCGTTTGACAATCCAACTTACGAGACTGGAGACACGAGAGAATAT | |
| | GAACTCTCCATCTAGGTGGGGGCAGTCTAGGGAAGTCAACTCAGACTTGCACCACAGTCC | |
| | AGCAGCAAGGCTCCTTGCTTCCTGCTGTCCCTCCACCTCCTGTATATACCACCTACGACG | |
| | AGATCCCACCAAGCCACTTTGTACATGTAATGTATTATATGGGGTCTGGGCTCCAGCCAG | |
| | AGAACAATCTTTTATTTCTGTTGTTTCCTTATTAAAATGGTGTTTTTGGAAAAAAAAAAA | |
| | AAAAAAAA | |
| | ORF Start: ATG at 178 | ORF Stop: TAG at 1753 |

| | | |
|---|---|---|
| SEQ ID NO: 164 | 525 aa | MW at 56462.7kD |

| NOV33d, | MRPVALLLLPSLLALLAHGLSLEAPTVGKGQAPGIEETDGELTAAPTPEQPERGVHFVTT | |
|---|---|---|
| CG52919-04 | | |
| Protein Sequence | APTLKLLNHHPLLEEFLQEGLEKGDEELRPALPFQPDPPAPFTPSPLPRLANQDSRPVFT | |
| | SPTPAMAAVPTQPQSKEGPWSPESESPMLRITAPLPPGPSMAVPTLGPGETASTTPPSRA | |
| | WTPTQEGPGDMGRPWVAEVVSQCAGIGIQGTITSSTASGDDEETTTTTTIITTTITTVQT | |
| | PGPCSWNFSGPEGSLDSPTDLSSPTDVGLDCFFYISVYPGYCVEIKVQNISLREGETVTV | |
| | EGLGGPDPLPLANQSFLLRGQVIRSPTHQAALRFQSLPPPAGPGTFHFHYQAYLLSCHFP | |
| | RRPAYGDVTVTSLHPGGSARFHCATGYQLKGARHLTCLNATQPFWDSKEPVCIAACGGVI | |
| | RNGTTGRIVSPGFPGNYSNNLTCHWLLEAPEGQRLHLHFEKVSLAEDDDRLIIRNGDNVE | |
| | APPVYDSYEVEYPPRPRPYNRITTESAFDNPTYETCETREYEVSI | |
| | SEQ ID NO: 165 | 2143 bp |

| NOV33e, | CCACGCCCTCGCCGTGGTGCTGATTCTGTCAGGCGCTGGCGGCGGCAGCGGCGGTGACGG | |
|---|---|---|
| CG52919-05 | | |
| DNA Sequence | CTGCGGCCCCGCTCCCTCTACCCGGCCGGACCCGGCTCTGCCCCCCCGCCCAAGCCCCAC | |
| | CAAGCCCCCCGCCCTCCCGCCGCGGTCCCAGCCCAGGGCGCGGCCGCAACCAGCACCATG | |
| | CGCCCGGTAGCCCTGCTGCTCCTGCCCTCGCTGCTGGCGCTCCTGGCTCACGGACTCTCT | |
| | TTAGAGGCCCCAACCGTGGGGAAAGGACAAGCCCCAGGCATCGAGGAGACAGATGGCGAG | |
| | CTGACAGCAGCCCCCACACCTGAGCAGCCAGAACGAGGCGTCCACTTTGTCACAACAGCC | |
| | CCCACCTTGAAGCTGCTCAACCACCACCCGCTGCTTGAGGAATTCCTACAAGAGGGGCTG | |
| | GAAAAGGGAGATGAGGAGCTGAGGCCAGCACTGCCCTTCCAGCCTCACCCACCTGCACCC | |
| | TTCACCCCAAGTCCCCTTCCCCGCCTGGCCAACCAGGACAGCCGCCCTGTCTTTACCAGC | |
| | CCCACTCCAGCCATGGCTGCGGTACCCACTCAGCCCCAGTCCAAGGAGGGACCCTGGAGT | |
| | CCGGACTCAGAGTCCCCTATGCTTCGAATCACAGCTCCCCTACCTCCAGGGCCCAGCATG | |
| | GCAGTGCCCACCCTAGGCCCAGGGGAGATAGCCAGCACTACACCCCCCAGCAGAGCCTGG | |
| | ACACCAACCCAAGAGGGTCCTGGAGACATGGGAAGGCCCTGGGTTGCAGAGGTTGTGTCC | |
| | CAGGGCGCAGGGATCGGGATCCAGGGGACCATCACCTCCTCCACAGCTTCAGGAGATGAT | |
| | GAGGAGACCACCACTACCACCACCATCATCACCACCACCATCACCACAGTCCAGACACCA | |
| | GGCCCTTGTAGCTGGAATTTCTCAGGCCCAGAGGGCTCTCTGGACTCCCCTACAGACCTC | |
| | AGCTCCCCCACTGATGTTGGCCTGGACTGCTTCTTCTACATCTCTGTCTACCCTGCCTAT | |
| | GGCGTGGAAATCAAGGTCCACAATATCAGCCTCCGGGAAGGGCAGACAGTGACTGTGGAA | |
| | GGCCTGGGGGGGCCTGACCCACTGCCCCTGGCCAACCAGTCTTTCCTGCTGCGGGGCCAA | |
| | GTCATCCGCAGCCCCACCCACCAAGCGGCCCTGAGGTTCCAGAGCCTCCCGCCACCGGCT | |
| | GGCCCTGGCACCTTCCATTTCCATTACCAAGCCTATCTCCTGAGCTGCCACTTTCCCCGT | |
| | CGTCCAGCTTATGGAGATGTGACTGTCACCAGCCTCCACCCAGGGGGTAGTGCCCGCTTC | |
| | CATTGTGCCACTGGCTACCAGCTGAAGGGCGCCAGGCATCTCACCTGTCTCAATGCCACC | |
| | CAGCCCTTCTGGGATTCAAAGGAGCCCGTCTGCATCGCTGCTTGCGGCGGAGTGATCCGC | |
| | AATGGCACCACCGGCCGCATCGTCTCTCCAGGCTTCCCGGGCAACTACAGCAACAACCTC | |
| | ACCTCTCACTGGCTCCTTGAGGCTCCTGAGGGCCAGCGGCTACACCTGCACTTTGAGAAG | |
| | GTTTCCCTGGCAGAGGATCATCACACGCTCATCATTCGCAATCGGGACAACGTGCAGGCC | |
| | CCACCAGTGGGAAAAAGCTCCCTGCAGCTGCCCCGCCCCCGCCCCCGCCCCTACAACCGC | |
| | ATTACCATAGAGTCAGCGTTTGACAATCCAACTTACGAGACTGGATCTCTTTCCTTTGCA | |
| | GGACACGAGACAATATGAAGTCTCCATCTAGGTGGGGGCAGTCTAGGGAAGTCAACTCAG | |
| | ACTTGCACCACAGTCCAGCAGCAAGGCTCCTTGCTTCCTGCTGTCCCTCCACCTCCTGTA | |
| | TATACCACCTAGGAGGAGATCCCACCTAGCCCTCAAGAAGTTGTGCCCTTCCCCGCCTGC | |
| | GATGCCCACCATGGCCTATTTTCTTGGTGTCATTGCCCACTTGGGGCCCTTGCATTCCGC | |
| | CATGTACACGGGGCATCTACCTCTGGCGAACAACATAGCTCGGACCACAAGCTTCAACAG | |
| | CCAGCATTCCTTGACCCTCCTTCATGGCCCTGGGACCAGCCTGGGGAACACANTTAGGCA | |
| | GGACCAGCGAGTTACCTTGTTTCACATGACCACCAACCATTCC | |
| | ORF Start: ATG at 178 | ORF Stop: TGA at 1756 |

| | | |
|---|---|---|
| SEQ ID NO: 166 | 526 aa | MW at 56252.6kD |

| NOV33e, | MRPVALLLLPSLLALLAHGLSLEAPTVGKGQAPGIEETDGELTAAPTPEQPERGVHFVTT | |
|---|---|---|
| CG52919-05 | A | |
| Protein Sequence | APTLKLLNHHPLLEEFLQEGLEKGDEELRPALPFQPDPPAPFTPSPLPRLANQDSRPVFT | |
| | SPTPAMAAVPTQPQSKEGPWSPESESPMLRITAPLPPGPSMAVPTLGPGEIASTTPPSRA | |
| | WTPTQECPCDMGRPWVAEVVSQGACIGTQCTITSSTASGDDEETTTTTTIITTTITTVQT | |
| | PGPCSWNFSGPEGSLDSPTDLSSPTDVGLDCFFYISVYPGYGVEIKVQNISLRECETVTV | |
| | EGLGGPDPLPLANQSFLLRGQVIRSPTHQAALRFQSLPPPAGPGTFHFHYQAYLLSCHFP | |
| | RRPAYGDVTVTSLHPCGSARFHCATGYQLKGARHLTCLNATQPFWDSKEPVCIAACGGVI | |
| | RNGTTGRIVSPGPPGNYSNNLTCHWLLEAPEGQRLHLHPEKVSLAEDDDRLIIRNGDNVE | |
| | APPVGKSSLQLPRPRPRPYNRITIESAFDNPTYETGSLSFAGDERI | |
| | SEQ ID NO: 167 | 1694 bp |

| NOV33f, | CAGGGCGCGGCCGCAACCAGCACCATGCGCCCGGTAGCCCTGCTGCTCCTGCCCTCGCTG | |
|---|---|---|
| CG52919-06 | | |
| DNA Sequence | CTGGCGCTCCTGGCTCACGGACTCTCTTTAGAGGCCCCAACCGTCGGGAAAGGACAACCC | |
| | CCACGCATCGACCACACAGATGGCGAGCTGACAGCAGCCCCCACACCTGAGCACCCAGAA | |
| | CGAGGCCTCCACTTTGTCACAACAGCCCCCACCTTGAAGCTGCTCAACCACCACCCCCTG | |
| | CTTGAGGAATTCCTACAAGAGGCCCTCGAAAAGGCACATGAGGAGCTCAGCCCAGCACTG | |
| | CCCTTCCAGCCTGACCCACCTGCACCCTTCACCCCAAGTCCCCTTCCCCGCCTGGCCAAC | |
| | CAGGACAGCCGCCCTGTCTTTACCAGCCCCACTCCAGCCATGGCTGCGGTACCCACTCAC | |
| | CCCCAGTCCAAGGAGGGACCCTGGAGTCCGGAGTCAGAGTCCCCTATGCTTCGAATCACA | |
| | GCTCCCCTACCTCCAGGGCCCAGCATGGCAGTGCCCACCCTAGGCCCAGGGGAGATAGCC | |
| | AGCACTACACCCCCCAGCAGAGCCTGCACACCAACCCAACACGCTCCTCCACACATGGGA | |
| | ACCTCCTCCACAGCTTCAGGAGATGATGAGGAGACCACCACTACCACCACCATCATCACC | |
| | ACCACCATCACCACAGTCCAGACACCAGGCCCTTGTAGCTGGAATTTCTCAGGCCCAGAG | |
| | GGCTCTCTGGACTCCCCTACAGACCTCAGCTCCCCCACTGATGTTGGCCTGGACTGCTTC | |
| | TTCTACATCTCTGTCTACCCTGGCTATGGCGTGGAAATCAAGGTCCAGAATATCAGCCTC | |
| | CGGGAAGGGGAGACAGTGACTGTGGAAGGCCTGGGGGGGCCTGACCCACTGCCCCTGGCC | |
| | AACCACTCTTTCCTGCTGCGGGGCCAAGTCATCCCCACCCCCACCCACCAAGCGGCCCTG | |
| | AGGTTCCAGAGCCTCCCCCCACCGGCTGGCCCTGGCACCTTCCATTTCCATTACCAAGCC | |
| | TATCTCCTGACCTGCCACTTTCCCCGTCGTCCAGCTTATGGAGATGTGACTGTCACCAGC | |
| | CTCCACCCAGGGGGTAGTGCCCGCTTCCATTGTGCCACTGGCTACCAGCTGAAGGGCGCC | |
| | AGGCATCTCACCTGTCTCAATGTCACCCAGCCCTTCTGGGATTCAAAGGAGCCCGTCTGC | |
| | ATCGCTGCTTGCGGCGGAGTGATCCGCAATGCCACCACCGGCCGCATCGTCTCTCCAGGC | |
| | TTCCCGGGCAACTACAGCAACAACCTCACCTGTCACTGGCTGCTTGAGGCTCCTGAGGGC | |
| | CAGCGGCTACACCTGCACTTTGAGAAGGTTTCCCTGGCAGAGGATGATGACAGGCTCATC | |
| | ATTCGCAATGGGGACAACGTGGAGGCCCCACCAGTGTATGATTCCTATGAGGTGGAATAC | |
| | CTGCCCATTGAGGGCCTGCTCAGCTCTGGCAAACACTTCTTTGTTGAGCCCCGCCCCCGC | |
| | CCCCGCCCCTACAACCGCATTACCATAGAGTCACCGTTTGACAATCCAACTTACGAGACT | |
| | GGATCTCTTTCCCTTGCAGGAGACGAGAGAATATGAAGTCTCCATCTAGGTGGGGGCAGT | |
| | CTAGGGAAGTCAAC | |
| | ORF Start: ATG at 25 | ORF Stop: TGA at 1654 |

| | | |
|---|---|---|
| SEQ ID NO: 168 | 543 aa | MW at 58351.0kD |

| NOV33f, | MRPVALLLLPSLLALLAHGLSLEAPTVGKCQAPGIEETDGELTAAPTPEQPERGVHFVTT | |
|---|---|---|
| CG52919-06 | | |
| Protein Sequence | APTLKLLNHHPLLEEFLQEGLEKGDEELRPALPFQPDPPAPFTPSPLPRLANQDSRPVFT | |
| | SPTPAMAAVPTQPQSKEGFWSPESESPMLRTTAPLPPGPSMAVPTLGPGEIASTTPPSRA | |
| | PGPCSWNFSGPEGSLDSPTDLSSPTDVGLDCFFYISVYPGYGVEIKVQNISLREGETVTV | |
| | EGLGGPDPLPLANQSFLLRGQVIRSPTHQAALRFQSLPPPAGPGTFHFHYQAYLLSCHFP | |
| | RRPAYGDVTVTSLHPGGSARFHCATGYQLKGARHLTCLNVTQPFWDSKEPVCIAACGGVI | |
| | RNATTGRIVSPGFPCNYSNNLTCHWLLEAPEGQRLHLHFEKVSLAEDDDRLIIRNCDNVE | |
| | APPVYDSYEVEYLPIEGLLSSGKHFFVEPRPRPRPYNRITTESAFDNPTYETGSLSLAGD | |
| | ERI | |
| | SEQ ID NO: 169 | 1482 bp |

| NOV33g, | CCAGGCGCTGGCCGTGGTGCTGATTCTGTCAGGCGCTGGCGGCCGCAGCCGCGGTGACGG | |
|---|---|---|
| CG52919-01 | | |
| DNA Sequence | CTGCGGCCCCCCTCCCTCTACCCGGCCGGACCCGGCTCTGCCCCCGCGCCCAAGCCCCAC | |
| | CAACCCCCCCGCCCTCCCGCCGCCGTCCCAGCCCAGGGCGCGGCCGCAACCAGCACCATG | |
| | CGCCCGGTAGCCCTGCTGCTCCTGCCCTCGCTGCTGGCGCTCCTGGCTCACGGACTCTCT | |
| | TTAGACGCCCCAACCGTGGGGAAAGGACAAGCCCCAGGCATCGAGGAGACAGATGGCGAG | |
| | CTGACAGCAGCCCCCACACCTGAGCAGCCAGAACGAGGCGTCCACTTTGTCACAACAGCC | |
| | CCCACCTTGAAGCTGCTCAACCACCACCCGCTGCTTCAGGAATTCCTACAAGAGGGGCTG | |
| | GAAAAGGGACATGAGGAGCTCAGGCCAGCACTGCCCTTCCAGCCTGACCCACCTGCACCC | |
| | TTCACCCCAAGTCCCCTTCCCCGCCTGGCCAACCAGGACAGCCCCCCTGTCTTTACCACC | |
| | CCCACTCCAGCCATGGCTGCGGTACCCACTCAGCCCCAGTCCAAGGAGGGACCCTGGAGT | |
| | CCGGAGTCACAGTCCCCTATGCTTCGAATCACAGCTCCCCTACCTCCAGGGCCCAGCATG | |
| | GCAGTGCCCACCCTAGGCCCAGGGCAGATAGCCAGCACTACACCCCCCAGCAGAGCCTCG | |
| | ACACCAACCCAAGAGGGTCCTGGAGACATGGGAAGGCCGTGGGTTGCAGAGGTTGTGTCC | |
| | CAGGGCGCAGGCATCGGGATCCAGGGGACCATCACCTCCTCCACACCTTCACGAGATGAT | |
| | GAGGAGACCACCACTACCACCACCATCATCACCACCACCATCACCACAGTCCACACACCA | |
| | GGTCAGCTACCTGCTGGCTTGCAGATGTCGAAATGGGCATGGGGGAGGCTGCGGGGCCCC | |
| | TAAAAGCCTGTCTCTGACACTGTGCCACCCTGCCCTGCCCTTTCCCACCAACGGCCAGCC | |
| | TGCAGGAGGCATGTAGATTGGACCCAGATAGACCTGAGCTCAAATCCTGATTCTTCAGCC | |
| | AAGTACAGTGGCTCATCCCTGTAATCCCAGCACTTTGGGAGGCAGAGGCCAGTGGATCAT | |
| | CTGAGGTCAGGACTTCAAGACCCTCCTGGCCAACATGGCGAAACACCATCTCTACTAAAA | |
| | ATACAAAAATGAGCCGGCCATGGTGGTGGGCACCTGTAATCCCACCTACTCGGGAGGCTG | |
| | AGGCAGGAGAATCACTCAAACCTCCGAGGCAGACGTTGCAGTGAGCTCAGATTGCACCAT | |
| | TGCACTCCAGCCTGGGCAACAGAGCGAGACTCTGTCTCAAAAAAGAAAAAATCTTGATTC | |
| | TTCCAACTATAACATGACCCTAGCAATTCTATTTAACATCTCATCTCTGAGCCTCATCTG | |
| | AAAATGGCAATAAGAAAATAAACTTCTGGCTAGAAAAAAAAA | |
| | ORF Start: ATG at 178 | ORF Stop: TAA at 961 |

| | | |
|---|---|---|
| SEQ ID NO: 170 | 261 aa | MW at 27471.8kD |

| NOV33g, | MRPVALLLLPSLLALLAHGLSLEAPTVGKGQAPGIEETDCELTAAPTPEQPERGVHFVTT | |
|---|---|---|
| CG52919-01 | | |
| Protein Sequence | APTLKLLNHRPLLEEFLQEGLEKGDEELRPALPFQPDPPAPFTPSPLPRLANQDSRPVFT | |
| | SPTPAMAAVPTQPQSKEGPWSPESESPMLRITAPLPPGPSMAVPTLGPGEIASTTPPSRA | |
| | WTPTQEGPGDMGRPWVAEVVSQGAGIGIQGTITSSTASGDDEETTTTTTIITTTITTVQT | |
| | PGQLPAGLQMWKWGWGRLRGP | |
| | SEQ ID NO: 171 | 840 bp |

| NOV33h, | TCCAGCCCGCGGCCGCAACCAGCACCATGCGCCCGGTAGCCCTGCTGCTCCTGCCCTCGC | |
|---|---|---|
| CG52919-07 | | |
| DNA Sequence | TGCTGGCGCTCCTGGCTCACGGACTCTCTTTAGAGGCCCCAACCGTGGGGAAAGGACAAG | |
| | CCCCAGGCATCGAGCAGACAGATGGCGAGCTGACACCAGCCCCCACACCTGAGCAGCCAG | |
| | AACGAGGCGTCCACTTTGTCACAACAGCCCCCACCTTGAAGCTGCTCAACCACCACCCGC | |
| | TGCTTGAGGAATTCCTACAAGAGGGGCCGCAAAAGGGAGATGAGGACCTGACGCCAGCAC | |
| | TGCCCTTCCAGCCTGACCCACCTGCACCCTTCACCCCAAGTCCCCTTCCCCCCCTGGCCA | |
| | ACCAGGACAGCCGCCCTGTCTTTACCAGCCCCACTCCAGCCATGCCTGCGGTACCCACTC | |
| | AGCCCCAGTCCAAGGAGGGACCCTGGAGTCCGGAGTCAGAGTCCCCTATGCTTCGAATCA | |
| | CAGCTCCCCTACCTCCACCGCCCAGCATGGCAGTGCCCACCCTAGGCCCAGGGGAGATAG | |
| | CCAGCACTACACCCCCCACCACAGCCTGGACACCAACCCAAGAGGGTCCTGGAGACATGC | |
| | GAAGGCCGTGGGTTGCAGAGGTTGTGTCCCAGGGCGCAGGGATCGGGATCCAGGGGACCA | |
| | TCACCTCCTCCACACCTTCAGGACATCATGAGCAGACCACCACTACCACCACCATCATCA | |
| | CCACCACCATCACCACAGTCCAGACACCAGGTCAGCTACCTGCTCCCTTGCAGATGTGGA | |
| | AATGGGGATGCGGGACGCTCCGGGGCCCCTAAAACCCTGTCTCTGACACTGTGCCAGCCA | |
| | ORF Start: ATG at 27 | ORF Stop: TAA at 810 |

| | | |
|---|---|---|
| SEQ ID NO: 172 | 261 aa | MW at 27455.8kD |

| NOV33i | MRPVALLLLPSLLALLAHGLSLEAPTVGKGQAPGIEETDGELTAAPTPEQPERGVHFVTT | |
|---|---|---|
| CG52919-07 | | |
| Protein Sequence | APTLKLLNHHPLLEEFLQEGPEKGDEELRPALPFQPDPPAPFTPSPLPRLANQDSRPVFT | |
| | SPTPAMAAVPTQPQSKEGPWSPESESPMLRITAPLPPGPSMAVPTLGPGEIASTTPPSRA | |
| | WTPTQEGPGDMGRPWVAEVVSQGACICIQGTITSSTASCDDEETTTTTTIITTTITTVQT | |
| | PGQLPAGLQMWKWGWGRLRGP | |
| | SEQ ID NO: 173 | 1654 bp |

| NOV33j | CACCAGATCTCCCACCATGCGCCCCGTAGCCCTGCTGCTCCTGCCCTCCCTGCTGGCCCT | |
|---|---|---|
| CG52919-08 | | |
| DNA Sequence | CCTGGCTCACGGACTCTCTTTAGAGGCCCCAACCGTGGCGAAACGACAAGCCCCAGGCAT | |
| | CGAGGAGACAGATGGCGACCTGACAGCAGCCCCCACACCTGACCAGCCAGAACGAGGCGT | |
| | CCACTTTGTCACAACAGCCCCCACCTTGAAGCTGCTCAACCACCACCCGCTCCTTGAGGA | |
| | ATTCCTACAAGAGGGGCTGCAAAAGCGACATGAGGAGCTGAGGCCAGCACTCCCCTTCCA | |
| | GCCTGACCCACCTGCACCCTTCACCCCAAGTCCCCTTCCCCGCCTCGCCAACCAGCACAG | |
| | CCGCCCTCTCTTTACCAGCCCCACTCCAGCCATGGCTGCGGTACCCACTCAGCCCCAGTC | |
| | CAAGGAGCGACCCTGGAGTCCGGAGTCAGAGTCCCCTATCCTTCCAATCACAGCTCCCCT | |
| | ACCTCCAGGGCCCAGCATGGCACTGCCCACCCTAGGCCCAGGGGACATAGCCAGCACTAC | |
| | ACCCCCCAGCAGAGCCTGGACACCAACCCAAGAGGGTCCTGGAGACATGGGAAGGCCGTG | |
| | GGTTGCAGAGGTTGTGTCCCAGGGCGCAGGGATCGGGATCCAGGGGACCATCACCTCCTC | |
| | CACACCTTCAGGACATCATGAGGAGACCACCACTACCACCACCATCATCACCACCACCAT | |
| | CACCACACTCCAGACACCAGGCCCTTGTAGCTGGAATTTCTCAGGCCCACACCCTTCTCT | |
| | GGACTCCCCTACAGACCTCAGCTCCCCCACTGATGTTGGCCTGGACTGCTTCTTCTACAT | |
| | CTCTGTCTACCCTGGCTATCGCGTGGAAATCAAGGTCCAGAATATCAGCCTCCGGGAAGG | |
| | GGACACAGTCACTGTGGAAGGCCTGGGGGGGCCTGACCCACTGCCCCTGGCCAACCAGTC | |
| | TTTCCTGCTGCGGCGCCAAGTCATCCGCAGCCCCACCCACCAAGCGGCCCTCAGGTTCCA | |
| | GAGCCTCCCGCCACCGGCTCGCCCTGGCACCTTCCATTTCCATTACCAAGCCTATCTCCT | |
| | GAGCTCCCACTTTCCCCCTCCTCCAGCTTATGGAGATGTGACTGTCACCAGCCTCCACCC | |
| | AGGGGGTAGTGCCCGCTTCCATTGTGCCACTGGCTACCAGCTGAAGGGCGCCAGGCATCT | |
| | CACCTGTCTCAATGTCACCCAGCCCTTCTGGGATTCAAAGGAGCCCGTCTGCATCGCTGC | |
| | TTGCGGCGGAGTGATCCGCAATGCCACCACCGCCCGCATCGTCTCTCCAGGCTTCCCCGG | |
| | CAACTACAGCAACAACCTCACCTGTCACTGGCTGCTTGAGGCTCCTGAGGGCCAGCGGCT | |
| | ACACCTGCACTTTGACAAGGTTTCCCTGGCAGAGGATGATGACAGGCTCATCATTCGCAA | |
| | TGGGGACAACGTGGACCCCCCACCAGTGTATGATTCCTATGAGGTGGAATACCTGCCCAT | |
| | TGAGGGCCTGCTCAGCTCTGGCAAACACTTCTTTCTTGACCCCCGCCCCCGCCCCCGCCC | |
| | CTACAACCGCATTACCATAGACTCAGCGTTTCACAATCCAACTTACGAGACTCGATCTCT | |
| | TTCCCTTGCAGGAGACGAGAGAATACTCGAGGGC | |
| | ORF Start: ATG at 17 | ORF Stop: at 1646 |

| | | |
|---|---|---|
| SEQ ID NO: 174 | 543 aa | MW at 58351.0kD |

| NOV33i, | MRPVALLLLPSLLALLAHGLSLEAPTVGKGQAPGIEETDGELTAAPTPEQPERGVHFVTT | |
|---|---|---|
| CG52919-08 | | |
| Protein Sequence | APTLKLLNHHPLLEEFLQEGLEKGDEELRPALPFQPDPPAPFTPSPLPRLANQDSRPVFT | |
| | SPTPAMAAVPTQPQSKEGPWSPESESPMLRITAPLPPGPSMAVPTLGPCEIASTTPPSRA | |
| | WTPTQEGPGDMGRPWVAEVVSQGAGIGTQGTITSSTASGDDEETTTTTTIITTTITTVQT | |
| | PGPCSWNFSGPEGSLDSPTDLSSPTDVGLDCFFYISVYPGYGVEIKVQNISLREGETVTV | |
| | EGLCGPDPLPLANQSFLLRGQVIRSPTHQAALRFQSLPPPAGPGTFHFHYQAYLLSCHFP | |
| | RRPAYCDVTVTSLHPGGSARFICATGYQLKGARHLTCLNVTQPFWDSKEPVCIAACGGVI | |
| | RNATTGRIVSPGFPCNYSNNLTCNWLLEAPEGQRLHLHFEKVSLAEDDDRLIIRNGDNVE | |
| | APPVYDSYEVEYLPIEGLLSSGKHFFVEPRPRPRPYNRITIESAFDNPTYETGSLSLAGD | |
| | ERI | |
| | SEQ ID NO: 175 | 1591 bp |

| NOV33j, | CACCAGATCTCTCTCTTTAGAGCCCCCAACCGTGGGGAAAGGACAAGCCCCAGGCATCGA | |
|---|---|---|
| CG52919-09 | | |
| DNA Sequence | GGAGACAGATGGCGAGCTCACAGCAGCCCCCACACCTGAGCACCCAGAACGAGGCGTCCA | |
| | CTTTGTCACAACAGCCCCCACCTTGAACCTGCTCAACCACCACCCGCTGCTTGAGGAATT | |
| | CCTACAAGAGGGGCTGGAAAAGGGAGATGAGGAGCTGAGGCCAGCACTGCCCTTCCAGCC | |
| | TGACCCACCTGCACCCTTCACCCCAAGTCCCCTTCCCCGCCTGGCCAACCAGGACAGCCG | |
| | CCCTGTCTTTACCACCCCCACTCCAGCCATGGCTGCCGTACCCACTCAGCCCCAGTCCAA | |
| | TCCAGCGCCCACCATGGCACTGCCCACCCTAGCCCCAGGGGACATACCCAGCACTACACC | |
| | CCCCAGCAGAGCCTGGACACCAACCCAAGAGGGTCCTGGAGACATGGGAAGGCCGTGGGT | |
| | TGCACAGGTTGTGTCCCACCCCGCAGCGATCGGGATCCAGGGGACCATCACCTCCTCCAC | |
| | AGCTTCACCAGATCATGAGGAGACCACCACTACCACCACCATCATCACCACCACCATCAC | |
| | CACAGTCCAGACACCAGGCCCTTGTAGCTGCAATTTCTCAGGCCCAGAGGGTTCTCTGGA | |
| | CTCCCCTACAGACCTCAGCTCCCCCACTGATGTTGGCCTCCACTGCTTCTTCTACATCTC | |
| | TGTCTACCCTGGCTATGGCGTGGAAATCAAGGTCCAGAATATCAGCCTCCGGGAAGGGGA | |
| | GACAGTGACTCTGGAAGGCCTGGGGCGGCCTGACCCACTCCCCCTGGCCAACCACTCTTT | |
| | CCTCCCGCCACCGGCTGGCCCTGGCACCTTCCATTTCCATTACCAAGCCTATCTCCTGAG | |
| | CTGCCACTTTCCCCGTCGTCCAGCTTATGGAGATGTGACTGTCACCAGCCTCCACCCACG | |
| | GGGTAGTGCCCGCTTCCATTGTGCCACTGGCTACCAGCTGAAGGGCGCCAGGCATCTCAC | |
| | CTGTCTCAATGTCACCCAGCCCTTCTGCGATTCAAAGGAGCCCGTCTGCATCGCTGCTTG | |
| | CTACAGCAACAACCTCACCTGTCACTGGCTCCTTCAGGCTCCTGAGGGCCAGCGGCTACA | |
| | CCTGCACTTTGAGAAGGTTTCCCTGCCAGAGCATGATCACAGGCTCATCATTCGCAATGG | |
| | GGACAACGTGGAGGCCCCACCAGTGTATGATTCCTATGAGCTGGAATACCTGCCCATTGA | |
| | GGGCCTGCTCAGCTCTGGCAAACACTTCTTTGTTGAGCCCCGCCCCCGCCCCCGCCCCTA | |
| | CAACCGCATTACCATAGAGTCAGCGTTTGACAATCCAACTTACGAGACTGGATCTCTTTC | |
| | CCTTGCAGGAGACGAGAGAATACTCGAGGGC | |
| | ORF Start: at 2 | ORF Stop: at 1583 |

| | | |
|---|---|---|
| SEQ ID NO: 176 | 527 aa | MW at 56714.8kD |

| | |
|---|---|
| NOV33j, | TRSLSLEAPTVGKGQAPGIEETDGELTAAPTPEQPERGVHFVTTAPTLKLLNHHPLLEEP |
| CG52919-09 | |
| Protein Sequence | LQEGLEKGDEELRPALPFQPDPPAPFTPSPLPRLANQDSRPVFTSPTPAMAAVPTQPQSK |
| | EGPWSPESESPMLRITAPLPPGPSMAVPTLGPGEIASTTPPSRAWTPTQEGPGDMGRPWV |
| | AEVVSQGAGIGIQGTITSSTASGDDEETTTTTTIITTTITTVQTPGPCSWNFSGPEGSLD |
| | SPTDLSSPTDVGLDCFFYISVYPGYCVEIKVQNISLREGETVTVECLGGPDPLPLANQSF |
| | LLRGQVIRSPTHQAALRFQSLPPPAGPGTFHFHYQAYLLSCHFPRRPAYGDVTVTSLHPG |
| | GSARFHCATGYQLKCARHLTCLNVTQPFWDSKEPVCIAACGGVIRNATTGRIVSPGFPCN |
| | YSNNLTCHWLLEAPEGQRLHLHFEKVSLAEDDDRLIIRNGDNVEAPPVYDSYEVEYLPIE |
| | GLLSSCKHFFVEPRPRPRPYNRITIESAFDNPTYETGSLSLAGDERI |

Sequence comparison of the above protein sequences yields the following sequence relationships shown in Table 33B.

TABLE 33B
Comparison of NOV33a against NOV33b through NOV33j.

| Protein | NOV33a Residues/ | Identities/ |
|---|---|---|
| Sequence | Match Residues | Similarities for the Matched Region |
| NOV33b | 1 . . . 242 | 166/242 (68%) |
| | 1 . . . 242 | 166/242 (68%) |
| NOV33c | 1 . . . 242 | 166/242 (68%) |
| | 1 . . . 242 | 166/242 (68%) |
| NOV33d | 1 . . . 242 | 166/242 (68%) |
| | 1 . . . 242 | 166/242 (68%) |
| NOV33e | 1 . . . 242 | 166/242 (68%) |
| | 1 . . . 242 | 166/242 (68%) |
| NOV33f | 1 . . . 242 | 166/242 (68%) |
| | 1 . . . 242 | 166/242 (68%) |
| NOV33g | 1 . . . 261 | 185/261 (70%) |
| | 1 . . . 261 | 185/261 (70%) |
| NOV33h | 1 . . . 261 | 184/261 (70%) |
| | 1 . . . 261 | 184/261 (70%) |
| NOV33i | 1 . . . 242 | 166/242 (68%) |
| | 1 . . . 242 | 166/242 (68%) |
| NOV33j | 20 . . . 242 | 167/223 (74%) |
| | 4 . . . 226 | 167/223 (74%) |

Further analysis of the NOV33a protein yielded the following properties shown in Table 33C.

TABLE 33C
Protein Sequence Properties NOV33a

| | |
|---|---|
| PSort | 0.8200 probability located in outside; 0.1000 probability |
| analysis: | located in endoplasmic reticulum (membrane); |
| | 0.1000 probability located in endoplasmic reticulum (lumen); |
| | 0.1000 probability located in lysosome (lumen) |
| SignalP | Cleavage site between residues 20 and 21 |
| analysis: | |

A search of the NOV33a protein against the Geneseq database, a proprietary database that contains sequences published in patents and patent publication, yielded several homologous proteins shown in Table 33D.

TABLE 33D
Geneseq Results for NOV33a

| | | NOV33a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAB70543 | Human PRO13 protein | 1 . . . 261 | 261/261 (100%) | e−154 |
| | sequence SEQ ID NO: 26 - | 1 . . . 261 | 261/261 (100%) | |
| | Homo sapiens, 261 aa. | | | |
| | [WO200110902-A2, | | | |
| | 15-FEB-2001] | | | |
| AAE15853 | Human SEZ6 protein - Homo | 1 . . . 242 | 242/242 (100%) | e−140 |
| | sapiens, 853 aa. | 1 . . . 242 | 242/242 (100%) | |
| | [WO200183552-A2, | | | |
| | 08-NOV-2001] | | | |
| AAU81976 | Human secreted protein | 1 . . . 242 | 242/242 (100%) | e−140 |
| | SECP2 - Homo sapiens, 994 | 1 . . . 242 | 242/242 (100%) | |
| | aa. [WO200198353-A2, | | | |
| | 27-DEC-2001] | | | |
| AAB70542 | Human PRO12 protein | 1 . . . 242 | 242/242 (100%) | e−140 |
| | sequence SEQ ID NO: 24 - | 1 . . . 242 | 242/242 (100%) | |
| | Homo sapiens, 526 aa. | | | |
| | [WO200110902-A2, | | | |
| | 15-FEB-2001] | | | |
| AAB70541 | Human PRO11 protein | 1 . . . 242 | 242/242 (100%) | e−140 |
| | sequence SEQ ID NO: 22 - | 1 . . . 242 | 242/242 (100%) | |
| | Homo Sapiens, 525 aa. | | | |
| | [WO200110902-A2, | | | |
| | 15-FEB-2001] | | | |

In a BLAST search of public sequence datbases, the NOV33a protein was found to have homology to the proteins shown in the BLASTP data in Table 33E.

TABLE 33E
Public BLASTP Results for NOV33a

| | | NOV33a | | |
|---|---|---|---|---|
| Protein | | Residues/ | Identities/ | |
| Accession | | Match | Similarities for the | Expect |
| Number | Protein/Organism/Length | Residues | Matched Portion | Value |
| CAC33421 | Sequence 25 from Patent | 1 . . . 261 | 261/261 (100%) | e−154 |
| | WO0110902 - Homo sapiens | 1 . . . 261 | 261/261 (100%) | |
| | (Human), 261 aa. | | | |
| CAC33420 | Sequence 23 from Patent | 1 . . . 242 | 242/242 (100%) | e−140 |
| | WO0110902 - Homo sapiens | 1 . . . 242 | 242/242 (100%) | |
| | (Human), 526 aa. | | | |
| CAC33418 | Sequence 19 from Patent | 1 . . . 242 | 242/242 (100%) | e−140 |
| | WO0110902 - Homo sapiens | 1 . . . 242 | 242/242 (100%) | |
| | (Human), 525 aa. | | | |
| CAC33417 | Sequence 17 from Patent | 1 . . . 242 | 242/242 (100%) | e−140 |
| | WO0110902 - Homo sapiens | 1 . . . 242 | 242/242 (100%) | |
| | (Human), 525 aa. | | | |
| CAC33416 | Sequence 15 from Patent | 1 . . . 242 | 242/242 (100%) | e−140 |
| | WO0110902 - Homo sapiens | 1 . . . 242 | 242/242 (100%) | |
| | (Human), 994 aa. | | | |

PFam analysis predicts that the NOV33a protein contains the domains shown in Table 33F.

TABLE 33F
Domain Analysis of NOV33a

| | | | |
|---|---|---|---|
| Pfam Domain | NOV33a | Identities/Similarities | Expect Value |
| | Match Region | for the Matched | |
| | | Region | |

### Example 34

The NOV34 clone was analyzed, and the nucleotide and encoded polypeptide sequences are shown in Table 34A.

TABLE 34A
NOV34 Sequence Analysis

| | |
|---|---|
| SEQ ID NO: 177 | 368 bp |

| | |
|---|---|
| NOV34a, | CTGTCCCACTCACCATGGAGAAGATCCTGATCCTGCTGCTTGTCGCCCTCTCTGTGGCCT |
| CG55698-01 | |
| DNA Sequence | ATGCAGCTCCTGGCCCCCGGGGCATCATTATCAACCTGGAGAACGGTGAGCTCTGCATGA |
| | ATAGTGCCCAGTGTAAGAGCAATTGCTGCCAGCATTCAAGTGCCCTGGGCCTGGCCCGCT |
| | GCACATCCATGGCCACCGAGAACAGCGAGTGCTCTGTCAACACGCTCTATGGCATTTACT |
| | ACAAGTGTCCCTGTGAGCGTCCCCTGACCTCTCAGGGAGACAAGACCATCGTGGGCTCCA |
| | TCACCAACACCAACTTTGGCATCTGCCATGACCCTGGACGCTCCAAGCAGTGAGACTGCC |
| | CACCCACT |

| | | |
|---|---|---|
| ORF Start: ATG at 15 | | ORF Stop: TGA at 351 |
| SEQ ID NO: 178 | 112 aa | MW at 11953.7 kD |

| | |
|---|---|
| NOV34a | MEKILILLLVALSVAYAAPCPRGIIINLENGELCMNSAQCKSNCCQHSSALGLARCTSMA |
| CG55698-01 | |
| Protein Sequence | SENSECSVKTLYGIYYKCPCERGLTCEGDKTIVGSTTNTNFGICHDAGRSKQ |

| | |
|---|---|
| SEQ ID NO: 179 | 394 bp |

| | |
|---|---|
| NOV34b, | AGCTGTCCCACTCGCCATGGAGAAGATCCTGATCCTCCTCCTTGTCGCCCTCTCTGTGGC |
| CG55698-02C | |
| DNA Sequence | CTATGCAGCTCCTGGCCCCCGCGCGATCATTATCAACCTCACGCTCTATGGCATTTACTA |
| | CAAGTGTCCCTGTGAGCGTGGCCTGACCTGTGAGCGACACAAGACCATCGTGGGCTCCAT |
| | CACCAACACCAACTTTGGCATCTGCCATGACGCTGGACGCTCCAAGCAGTGAGACTGCCC |
| | ACCCACTCCCACACCTAGCCCAGAATGCTGTAGGCCACTACGCCCAGGGGCATCTCTCCC |
| | CTGCTCCAGCGCATCTCCCGGCCTGGCCACCTCCTTCACCAGCATATCTGTTTTCTGATT |
| | GCGCTCTTCACAATTAAAGGCCTCCTCCAAACCT |

| | | |
|---|---|---|
| ORF Start: ATG at 17 | | ORF Stop: TGA at 230 |
| SEQ ID NO: 180 | 71 aa | MW at 7658.9 kD |

| | |
|---|---|
| NOV34b | MEKILILLLVALSVAYAAPGPRGIIINLTLYGIYYKCPCERGLTCEGDKTIVGSITNTNF |
| CG55698-02 | |
| Protein Sequence | GICHDAGRSKQ |

Sequence comparison of the above protein sequences yields the following sequence relationships shown in Table 34B.

TABLE 34B
Comparison of NOV34a against NOV34b.

| Protein | NOV34a Residues/ | Identities/Similarities |
|---|---|---|
| Sequence | Match Residues | for the Matched Region |
| NOV34b | 1 . . . 112 | 56/112 (50%) |
| | 1 . . . 71 | 56/112 (50%) |

Four polymorphic variants of NOV34b have been identified and are shown in Table 41M.

Further analysis of the NOV34a protein yielded the following properties shown in Table 34C.

TABLE 34C
Protein Sequence Properties NOV34a

| | |
|---|---|
| PSort analysis: | 0.8200 probability located in outside; 0.1000 |
| | probability located in endoplasmic reticulum |
| | (membrane); 0.1000 probability located in |
| | endoplasmic reticulum (lumen); 0.1000 |
| | probability located in lysosome (lumen) |
| SignalP analysis: | Cleavage site between residues 18 and 19 |

A search of the NOV34a protein against the Geneseq database, a proprietary database that contains sequences published in patents and patent publication, yielded several homologous proteins shown in Table 34D.

TABLE 34D
Geneseq Results for NOV34a

| | | NOV34a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAB54163 | Human pancreatic cancer | 1 . . . 112 | 112/112 (100%) | 3e−62 |
| | antigen protein sequence | 20 . . . 131 | 112/112 (100%) | |
| | SEQ ID NO: 615 - Homo | | | |
| | sapiens, 131 aa. | | | |
| | [WO200055320-A1, 21 | | | |
| | SEP. 2000] | | | |
| AAY91513 | Human secreted protein | 28 . . . 111 | 28/84 (33%) | 1e−07 |
| | sequence encoded by gene 63 | 33 . . . 114 | 38/84 (44%) | |
| | SEQ ID NO: 186 - Homo | | | |
| | sapiens, 122 aa. | | | |
| | [WO200006698-A1, 10 | | | |
| | FEB. 2000] | | | |
| AAY35930 | Extended human secreted | 28 . . . 111 | 28/84 (33%) | 1e−07 |
| | protein sequence, SEQ ID | 33 . . . 114 | 38/84 (44%) | |
| | NO. 179 - Homo sapiens, | | | |
| | 121 aa. [WO9931236-A2, | | | |
| | 24 JUN. 1999] | | | |
| AAB62640 | Human colipase-like protein- | 32 . . . 111 | 26/80 (32%) | 3e−07 |
| | 1 (Zclps1) - Homo sapiens, | 34 . . . 111 | 36/80 (44%) | |
| | 118 aa. [WO200136466-A2, | | | |
| | 25 MAY 2001] | | | |
| AAB62648 | Human colipase-like protein- | 32 . . . 109 | 25/78 (32%) | 1e−06 |
| | 1 (Zclps1) fragment - Homo | 22 . . . 97 | 35/78 (44%) | |
| | sapiens, 97 aa. | | | |
| | [WO200136466-A2, 25 | | | |
| | MAY 2001] | | | |

In a BLAST search of public sequence datbases, the NOV34a protein was found to have homology to the proteins shown in the BLASTP data in Table 34E.

TABLE 34E
Public BLASTP Results for NOV34a

| | | NOV34a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P04118 | Colipase precursor - Homo | 1 . . . 112 | 112/112 (100%) | 9e−62 |
| | sapiens (Human), 112 aa. | 1 . . . 112 | 112/112 (100%) | |
| P19090 | Colipase precursor - Canis | 1 . . . 112 | 88/112 (78%) | 6e−50 |
| | familiaris (Dog), 112 aa. | 1 . . . 112 | 99/112 (87%) | |
| P42890 | Colipase precursor - | 1 . . . 106 | 88/106 (83%) | 1e−49 |
| | Oryctolagus cuniculus | 1 . . . 106 | 97/106 (91%) | |
| | (Rabbit), 107 aa. | | | |
| Q91XL7 | Pancreatic colipase - | 3 . . . 112 | 87/110 (79%) | 2e−49 |
| | Spermophilus | 2 . . . 111 | 100/110 (90%) | |
| | tridecemlineatus (Thirteen- | | | |
| | lined ground squirrel), 111 aa. | | | |
| Q9N1T6 | Colipase - Sus scrofa (Pig), | 1 . . . 110 | 86/110 (78%) | 1e−48 |
| | 112 aa. | 1 . . . 110 | 95/110 (86%) | |

PFam analysis predicts that the NOV34a protein contains the domains shown in Table 34F.

TABLE 34F
Domain Analysis of NOV34a

| Pfam | NOV34a | Identities/Similarities | Expect |
|---|---|---|---|
| Domain | Match Region | for the Matched Region | Value |
| Colipase | 21 . . . 60 | 32/40 (80%) | 5.5e−24 |
| | | 40/40 (100%) | |
| Colipase_C | 62 . . . 106 | 41/47 (87%) | 3.2e−34 |
| | | 45/47 (96%) | |

Pancreatic lipase catalyzes the hydrolysis triacylglycerol to fatty acids. These triacylglycerides are present predominantly as an emulsified micelle stabilized by bile acids. Since lipase hydrolizes the ester linkage of triacylglyceride, the active site must be positioned at the bile salt-coated water-lipid interface of this micelle. Since the bile salts can inhibit lipase, colipase is secreted to anchor the lipase to the water-lipid interface so that hydrolysis can occur.

Table 34G shows an alignment of the porcine pancreatic colipase (Q9N 1T6; SEQ ID NO:797) with the splice variant NOV34b (CG55698-02; SEQ ID NO:180). The arrow indicates the signal sequence cleavage site. Since the homology between the porcine and human lipases is high, the x-ray crystal structure of the porcine lipase is a suitable comparison for the effects of NOV34b (CG55698-02).

[image]

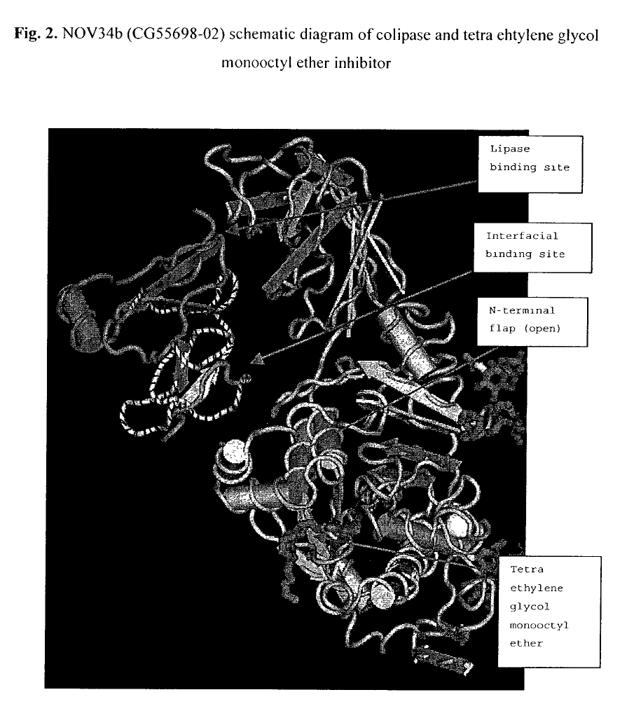

FIG. 2 shows the x-ray crystal structure (1ETH) at a 2.84 Å resolution of poricine lipase (right) with colipase (left) (Hermoso, et. al, J. Biol. Chem., 2001, 271:1807-18016). The tetra ethylene glycol monooctyl ether inhibitor is shown in the active site of lipase. The deleted sequence found in NOV34b is indicated with hatch marks.

The amino-terminal domain of lipase contains the active site whereas the carboxy-terminal domain binds to colipase. Likewise, colipase possesses a lipase binding domain and a micelle interfacial binding site. The catalytic site of lipase is inaccessible in solution since there is an N-terminal flap which covers the active site, preventing substrate from entering. The colipase additionally serves to stabilize the active form of lipase by binding to the N-terminal flap and thus keeping it in an open, active conformation which allows substrate to enter the lipase active site.

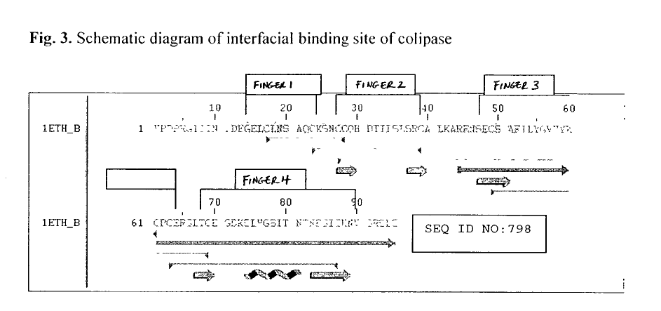

The interfacial binding site of colipase is composed of four hydrophobic fingers (finger1:14-24, finger2:27-39, finger3:47-64, and finger4: 68-90 numbered according to the colipase sequence in FIG. 3). In NOV34b, Fingers 1, 2 and a portion of 3 are missing suggesting that the splice variant would be less adept at binding the micelle interface.

Of the 8 polar interactions (includes hydrogen bonds and salt bridges) between lipase and colipase, 5 of bind to the C-terminal region of lipase and the remainder bind to the N-terminal flap. Of these, only one of the 5 bonds NOV34b:C-terminal bonds is missing, but all three of the NOV34b:N-teriminal flap bonds missing. Of the 17 colipase:lipase van der Waals contacts, 4 of these contact the N-terminal flap and the remainder bond to the C-terminal domain. For NOV34b, 11 of the 13 van der Waals contacts to the lipase C-terminal domain and none of the N-terminal flap contacts are present. Of the 4 bridging water contacts at the colipase:lipase C-terminal binding site, 2 are lost in NOV34b.

The splice variant NOV34b retains most of the binding sites to the C-terminal of lipase, but are missing half of the micelle interfacial binding domain and the entire N-terminal flap binding site. NOV34b may still bind to lipase, but may not anchor it to the micelle interface very well and would not be able to stabilize the open, active formation of lipase (since it cannot bind the N-terminal flap). Thus, it is possible that NOV34b may compete for binding with the normal, lipase-activating form of colipase to lipase. Since the NOV34b lipase complex fails to position the N-terminal flap away from the active site of lipase and thus prevents substrate binding, NOV34b may be considered to be a competitive inhibitor of the lipase enzymatic activity.

### Example 35

The NOV35 clone was analyzed, and the nucleotide and encoded polypeptide sequences are shown in Table 35A.

TABLE 35A
NOV35 Sequence Analysis

| | |
|---|---|
| SEQ ID NO: 181 | 7286 bp |

| | |
|---|---|
| NOV35a, | GAATTCGCTAGAGCCCTAGAGCCCCAGCAGCACCCAGCCAAACCCACCTCCACCATGCGG |
| CG55832-01 | |
| DNA Sequence | GCCATGACTCAGCTGTTGGCAGCTGTCTTTCTTGCTTTCCTTGCCCTCGCTACCGAAGGT |
| | GGGGTCCTCAAGAAAGTCATCCGGCACAAGCGACAGAGTGGGCTGAACGCCACCCTGCCA |
| | GAAGAGAACCAGCCAGTGGTGTTTAACCACGTTTACAACATCAAGCTGCCAGTGGGATCC |
| | CAGTGTTCGGTGGATCTGGAGTCAGCCAGTGGGGAGAAAGACCTGGCACCGCCTTCAGAG |
| | CCCAGCGAAAGCTTTCAGGAGCACACAGTAGATGGGGAAAACCAGATTGTCTTCACACAT |
| | CGCATCAACATCCCCCGCCGGGCCTGTGGCTGTGCCGCAGCCCCTGATGTTAAGGAGCTG |
| | CTGAGCAGACTGGAGGAGCTGGAGAACCTGGTGTCTTCCCTGAGGGAGCAATGTACTGCA |
| | GGAGCACGCTGCTGTCTCCAGCCTGCCACACGCCGCTTGGACACCAGGCCCTTCTGTAGC |
| | GGTCGGGGCAACTTCAGCACTGAAGGATGTGGCTGTGTCTGCGAACCTGGCTGGAAAGGC |
| | CCCAACTGCTCTGAGCCCGAATGTCCAGGCAACTGTCACCTTCGAGGCCGGTCCATTGAT |
| | GGGCAGTGCATCTGTGACGACGGCTTCACGGGCCAGGACTGCAGCCAGCTGGCTTGCCCC |
| | AGCGACTGCAATGACCAGGGCAAGTGCGTGAATGGAGTCTGCATCTGTTTCGAAGGCTAC |
| | GCGGCTGACTGCAGCCGTGAAATCTGCCCAGTGCCCTGCAGTGAGGAGCACGGCACATGT |
| | GTAGATGGCTTGTGTGTGTCCCACGATGGCTTTGCACGCGATGACTGCAACAAGCCTCTG |
| | TGTCTCAACAATTGCTACAACCGTGGACGATGCGTGGAGAATGAGTGCGTGTGTGATGAG |
| | GCTTTCACGGGCGAAGACTGCAGTGAGCTCATCTGCCCCAATGACTGCTTCGACCGGGCC |
| | CGCTGCATCAATGGCACCTGCTACTGCGAAGAACGCTTCACAGGTGAAGACTGCGGGAAA |
| | CCCACCTGCCCACATGCCTGCCACACCCAGGGCCGGTGTGAGGAGGGGCAGTGTGTATGT |
| | GATGAGGGCTTTGCCGGTGTGCACTGCAGCGAGAAGAGGTGTCCTGCTGACTGTCACAAT |
| | CGTGGCCGCTGTGTACACGGGCGGTGTGAGTGTGATGATGGTTTCACTGGAGCTGACTGT |
| | GGGGAGCTCAACTGTCCCAATGGCTGCAGTGGCCATCGCCGCTGTGTCAATGGGCAGTGT |
| | GTGTGTGATGAGGGCTATACTGGGGAGGACTGCAGCCAGCTACGGTGCCCCAATCACTGT |
| | CACAGTCGGGGCCGCTGTGTCGAGGCCAAATGTGTATGTGAGCAACGCTTCAAGGCCTAT |
| | GACTGCAGTGACATCAGCTGCCCTAATGACTGTCACCAGCACGGCCGCTGTCTGAATGGC |
| | ATGTGTGTTTGTGATGACCGCTACACAGGGGAAGACTGCCGGGATCGCCAATGCCCCAGG |
| | GACTGCAGCAACAGGGCCCTCTGTGTGGACGGACAGTGCGTCTGTGAGGACGGCTTCACC |
| | GGCCCTGACTGTCCAGAACTCTCCTCTCCAAATGACTGCCATGGCCAGCGTCGCTCTGTG |
| | AATGGGCAGTGCGTGTGCCATGAAGGATTTATGGCCAAAGACTGCAAGGAGCAAAGATGT |
| | CCCAGTGACTGTCATGGCCACGGCCGCTGCGTGCACCCCCAGTGCATCTGCCACGAGGGC |
| | TTCACAGGCCTCGACTGTGGCCAGCACTCCTGCCCCAGTGACTGCAACAACTTAGGACAA |
| | TGCGTCTCGGCCCGCTGCATCTGCAACGAGGGCTACAGCGGACAAGACTGCTCAGAGGTG |
| | TCTCCTCCCAAAGACCTCGTTGTGACAGAAGTCACGGAAGAGACGCTCAACCTGGCCTGG |
| | GACAATGAGATGCGGGTCACACAGTACCTTGTCCTGTACACGCCCACCCACGAGGGTGGT |
| | CTGGAAATGCAGTTCCGTGTGCCTGGGCACCAGACGTCCACCATCATCCGGCAGCTGGAG |
| | CCTGGTGTGCAGTACTTTATCCGTCTATTTCCCATCCTGGAGAACAAGAAGAGCATTCCT |
| | GTCAGCGCCAGGGTGGCCACGTACTTACCTGCACCTGAAGGCCTGAAATTCAAGTCCATC |
| | AAGGAGACATCTGTGGAAGTGGAGTGGGATCCTCTAGACATTGCTTTTGAAACCTGGGAG |
| | ATCATCTTCCGGAATATGAATAAAGAAGATGAGGGAGAGATCACCAAAAGCCTGAGGAGG |
| | CCAGAGACCTCTTACCGGCAAACTGGTCTACCTCCTCGCCAACAGTATGAGATATCTCTG |
| | CACATAGTGAAAAACAATACCCGGGGCCCTCGCCTGAAGAGGGTGACCACCACACGCTTG |
| | GATGCCCCCAGCCAGATCGAGGTGAAAGATGTCACAGACACCACTGCCTTGATCACCTGG |
| | TTCAAGCCCCTGGCTGAGATCGATGGCATTGACCTCACCTACGGCATCAAAGACGTGCCA |
| | GGAGACCGTACCACCATCGATCTCACAGAGGACGAGAACCAGTACTCCATCGGGAACCTG |
| | AAGCCTGACACTGAGTACCAGGTGTCCCTCATCTCCCGCACACGTGACATGTCAAGCAAC |
| | CCAGCCAAAGACACCTTCACAACAGGCCTCGATCCTCCCAGGAATCTTCGACGTGTTTCC |
| | CAGACAGATAACAGCATCACCCTGCAATGGAGGAATGGCAAGCCAGCTATTGACAGTTAC |
| | AGAATTAAGTATGCCCCCATCTCTGCAGGGGACCACGCTCAGGTTGATGTTCCAAAGAGC |
| | CAACAAGCCACAACCAAAACCACACTCACAGCTCTGAGGCCGGGAACTGAATATGGGATT |
| | GGACTTTCTGCTCTGAAGCAAGACAAGGAGAGCAATCCAGCGACCATCAACGCAGCCACA |
| | GAGTTGGACACGCCCAAGGACCTTCAGGTTTCTGAAACTGCACAGACCAGCCTGACCCTG |
| | CTCTGGAAGACACCGTTGGCCAAATTTGACCGCTACCGCCTCAATTACAGTCTCCCCACA |
| | GGCCAGTGGGTGGGAGTGCAGCTTCCAAGAAACACCACTTCCTATGTCCTGAGAGGCCTG |
| | GAACCAGGACACCAGTACAATGTCCTCCTGACAGCCCAGAAAGGCAGACACAAGAGCAAG |
| | CCCGCACGTGTGAAGGCATCCACTGAACAAGCCCCTGAGCTGGAAAACCTCACCGTGACT |
| | GAGGTTGGCTGGGATGGCCTCAGACTCAACTGGACCGCGGCTGACCAGGCCTATGAGCAC |
| | TTTATCATTCAGGTGCAGGAGGCCAACAAGGTGGAGGCAGCTCGGAACCTCACCGTGCCT |
| | GGCAGCCTTCGGGCTGTGGACATACCGGGCCTCAAGGCTGCTACGCCTTATACAGTCTCC |
| | ATCTATGGGGTGATCCAGGGCTATAGAACACCAGTGCTCTCTGCTGAGGCCTCCACAGGG |
| | GAAACTCCCAATTTGGGAGACGTCGTGGTGGCCGAGCTGGGCTGGGATGCCCTCAAACTC |
| | AACTGGACTGCTCCAGAAGGGGCCTATGAGTACTTTTTCATTCAGGTGCAGGAGGCTGAC |
| | ACAGTAGAGGCAGCCCAGAACCTCACCGTCCCAGCAGGACTGAGGTCCACAGACCTGCCT |
| | GGGCTCAAAGCAGCCACTCATTATACCATCACCATCCGCGGGGTCACTCAGGACTTCAGC |
| | ACAACCCCTCTCTCTGTTGAAGTCTTGACAGAGGAGGTTCCAGATATGGGAAACCTCACA |
| | GTGACCGAGGTTAGCTGGGATGCTCTCAGACTGAACTGGACCACGCCAGATGGAACCTAT |
| | GACCAGTTTACTATTCAGGTCCAGGAGGCTGACCAGGTCGAAGAGGCTCACAATCTCACG |
| | GTTCCTGGCAGCCTGCGTTCCATGGAAATCCCAGGCCTCAGGGCTGGCACTCCTTACACA |
| | GTCACCCTGCACGGCGAGGTCAGGGGCCACAGCACTCGACCCCTTGCTGTAGAGGTCGTC |
| | ACAGAGGATCTCCCACAGCTGGGAGATTTAGCCGTGTCTGAGGTTGGCTGGGATGGCCTC |
| | AGACTCAACTGGACCGCAGCTGACAATGCCTATGAGCACTTTGTCATTCAGGTGCAGCAG |
| | GTCAACAAAGTGGAGGCAGCCCAGAACCTCACGTTGCCTGGCAGCCTCACGGCTGTGCAC |
| | ATCCCGGGCCTCGAGGCTGCCACGCCTTATAGAGTCTCCATCTATGGGGTGATCCGCGGC |
| | TATAGAACACCAGTACTCTCTGCTGACGCCTCCACAGCCAAAGAACCTGAAATTGGAAAC |
| | TTAAATGTTTCTGACATAACTCCCGAGAGCTTCAATCTCTCCTGGATCGCTACCGATGGG |
| | ATCTTCGAGACCTTTACCATTGAAATTATTGATTCCAATAGGTTGCTGGAGACTGTGGAA |
| | TATAATATCTCTGGTGCTGAACGAACTGCCCATATCTCAGGGCTACCCCCTAGTACTGAT |
| | TTTATTGTCTACCTCTCTGGACTTGCTCCCAGCATCCGGACCAAAACCATCAGTGCCACA |
| | GCCACGACAGAGGCCCTGCCCCTTCTCGAAAACCTAACCATTTCCGACATTAATCCCTAC |
| | GGGTTCACAGTTTCCTGGATGGCATCGGAGAATGCCTTTGACAGCTTTCTAGTAACGGTG |
| | GTGGATTCTGGGAAGCTGCTGGACCCCCAGGAATTCACACTTTCAGGAACCCAGAGGAAG |
| | CTGGAGCTTAGAGGCCTCATAACTGGCATTGGCTATGACGTTATCGTCTCTGCCTTCACC |
| | CAAGGGCATCAAACCAAGCCCTTGAGGGCTGAGATTGTTACAGAAGCCGAACCGGAAGTT |
| | GACAACCTTCTGCTTTCACATGCCACCCCAGACGGTTTCCCTCTGTCCTGGACAGCTGAT |
| | GAAGGGGTCTTCGACAATTTTGTTCTCAAAATCAGAGATACCAAAAAGCAGTCTGAGCCA |
| | CTGGAAATAACCCTACTTGCCCCCGAACGTACCAGGGACATAACAGGTCTCACAGAGCCT |
| | ACTGAATACGAAATTCAACTCTATCGAATAAGCAAAGGAAGGCCATCCCAGACAGTCAGT |
| | GCTATAGCAACAACAGCCATGGGCTCCCCAAAGGAAGTCATTTTCTCAGACATCACTGAA |
| | AATTCGGCTACTGTCAGCTGGAGGGCACCCACGCCCCAAGTGCACACCTTCCQGATTACC |
| | TATGTGCCCATTACAGCAGGTACACCCTCCATGGTAACTCTGGACGGAACCAAGACTCAG |
| | ACCAGGCTGGTGAAACTCATACCTCGCGTGGAGTACCTTGTCAGCATCATCGCCATGAAG |
| | GGCTTTGAGGAAAGTGAACCTGTCTCAGCGTCATTCACCACACCTCTGGATCGCCCATCT |
| | GGCCTGGTGACAGCCAACATCACTGACTCAGAAGCCTTGGCCAGGTGGCAGCCACCCATT |
| | GCCACTGTGGACAGTTATGTCATCTCCTACACAGGCCAGAAAGTGCCAGAAATTACACGC |
| | ACGGTGTCCGGGAACACAGTGGAGTATGCTCTGACCCACCTCGAGCCTGCCACGGAATAC |
| | ACACTGAGAATCTTTGCAGAGAAAGGGCCCCACAAGAGCTCAACCATCACTGCCAAGTTC |
| | ACAACAGACCTCGATTCTCCAAGAGACTTGACTGCTACTGAGGTTCAGTCGGAAACTGCC |
| | CTCCTTACCTGGCGACCCCCCCGGCCATCAGTCACCGGTTACCTGCTCGTCTATGAATCA |
| | GTGGATGGCACAGTCAAGGAAGTCATTGTGCGTCCAGATACCACCTCCTACAGCCTGGCA |
| | GACCTGAGCCCATCCACCCACTACACAGCCAAGATCCAGCCACTCAATGGGCCCCTCAGG |
| | AGCAATATGATCCAGACCATCTTCACCACAATTGGACTCCTGTACCCCTTCCCCAAGGAC |
| | TGCTCCCAAGCAATGCTGAATCGACACACGACCTCTCGCCTCTACACCATTTATCTGAAT |
| | GGTGATAAGGCTCAGGCGCTGGAAGTCTTCTGTGACATCACCTCTGATCGGGGTGGATGG |
| | ATTGTGTTCCTGAGACGCAAAAACGGACGCGAGAACTTCTACCAAAACTGGAAGGCATAT |
| | GCTGCTGCATTTGGCGACCGCACAGAACAATTCTGGCTTGGGCTCGACAACCTGAACAAA |
| | ATCACAGCCCAGGGCCAGTACGAGCTCCGGGTGGACCTGCGGGACCATGCGCAGACAGCC |
| | TTTGCTCTCTATCACAAGTTCAGCGTGGGAGATGCCAAGACTCGCTACAAGCTGAAGGTG |
| | GAGGGGTACACTCGGACAGCAGGTGACTCCATGGCCTACCACAATGGCAGATCCTTCTCC |
| | ACCTTTGACAACGACACAGATTCAGCCATCACCAACTGTGCTCTGTCTACAAGGCGCTTC |
| | TGGTACAGGAACTGTCACCGTGTCAACCTGATGGGGACATATGGGGACAATAACCACAGT |
| | CAGCGCGTTAACTGCTTCCACTGGAAGGGCCACGAACACTCAATCCAGTTTGCTGAGATG |
| | AAGCTGAGACCAAGCAACTTCAGAAATCTTGAACGCAGGCGCAAACGGGCATAAATTGCA |
| | GGGACCACTGGGTGAGAGAGGAATAAGGCGGCCCAGAGCGAGGAAAGGATTTTACCAAAG |
| | CATCAATACAACCAGCCCAACCATCGGTCCACACCTGGGCATTTGGTGAGAATCAAAGCT |
| | GACCATGGATCCCTGGGGCCAACGGCAACAGCATGGGCCTCACCTCCTCTGTGATTTCTT |
| | TCTTTGCACCAAAGACATCAGTCTCCAACATGTTTCTGTTTTGTTGTTTGATTCAGCAAA |
| | AATCTCCCAGTGACAACATCGCAATAGTTTTTTACTTCTCTTAGGTGGCTCTGGGATGGG |
| | AGAGGGGTAGGATGTACAGGGGTAGTTTGTTTTAGAACCAGCCGTATTTTACATGAAGCT |
| | GTATAATTAATTGTCATTATTTTTCTTAGCAAAGATTAAATGTGTCATTGGAAGCCATCC |
| | CTTTTTTTACATTTCATACAACAGAAACCAGAAAAGCAATACTGTTTCCATTTTAAGGAT |
| | ATGATTAATATTATTAATATAATAATGATGATGATGATGATGAAAACTAAGGATTTTTCA |
| | AGAGATCTTTCTTTCCAAAACATTTCTGGACAGTACCTGATTGTATTTTTTTTTTAAATA |
| | AAAGCACAAGTACTTTTGAAAAAAAA |

| | | |
|---|---|---|
| ORF Start: ATG at 55 | | ORF Stop: TAA at 6652 |
| SEQ ID NO: 182 | 2199 aa | MW at 240715.6 kD |

| | |
|---|---|
| NOV35a, | MGAMTQLLAGVFLAFLALATEGGVLKKVIRHKRQSGVNATLPEENQPVVFNHVYNIKLPV |
| CG55832-01 | |
| Protein Sequence | GSQCSVDLESASGEKDLAPPSEPSESFQEHTVDGENQIVFTHRINIPRRACGCAAAPDVK |
| | ELLSRLEELENLVSSLREQCTAGAGCCLQPATGRLDTRPFCSGRGNFSTEGCGCVCEPGW |
| | KGPNCSEPECPGNCHLRGRCIDGQCICDDGFTGEDCSQLACPSDCNDQGKCVNGVCICFE |
| | GYAADCSREICPVPCSEEHGTCVDGLCVCHDGFAGDDCNKPLCLNNCYNRGRCVENECVC |
| | DEGFTGEDCSELICPNDCPDRGRCINGTCYCEECFTGEDCGKPTCPHACHTQGRCEEGQC |
| | VCDEGFAGVDCSEKRCPADCHNRGRCVDGRCECDDGFTGADCGELKCPNGCSGHGRCVNG |
| | QCVCDEGYTGEDCSQLRCPNDCHSRGRCVEGKCVCEQGFKGYDCSDMSCPNDCHQHGRCV |
| | NGMCVCDDGYTGEDCRDRQCPRDCSNRGLCVDGQCVCEDGFTGPDCAELSCPNDCHGQGR |
| | CVNGQCVCHEGFMGKDCKEQRCPSDCHGQGRCVDGQCICHEGFTGLDCCQHSCPSDCNNL |
| | GQCVSGRCICNEGYSGEDCSEVSPPKDLVVTEVTEETVNLAWDNEMRVTEYLVVYTPTHE |
| | GGLEMQFRVPGDQTSTIIRELEPGVEYFIRVFATLENKKSTPVSARVATYLPAPEGLKFK |
| | SIKETSVEVEWDPLDIAFETWEIIFRNMNKEDEGEITKSLRRPETSYRQTGLAPCQEYEI |
| | SLHIVKNNTRGPGLKRVTTTRLDAPSQIEVKDVTDTTALITWFKPLAEIDGIELTYGLKD |
| | VPGDRTTIDLTEDENQYSIGNLKPDTEYEVSLISRRGDMSSNPAKETFTTGLDAPRNLRR |
| | VSQTDNSITLEWRNGKAAIDSYRIKYAPISCGDHAEVDVPKSQQATTKTTLTGLRPGTEY |
| | GIGVSAVKSDKESNPATINAATELDTPKDLQVSETAETSLTLLWKTPLAKFDRYRLNYSL |
| | PTGQWVGVQLPRNTTSYVLRGLEPGQEYNVLLTAEKGRHKSKPARVKASTEQAPELENLT |
| | VTEVGWDGLRLNWTAADQAYEHFIIQVQEANKVEAARNLTVPGSLRAVDTPGLKAATPYT |
| | VSIYGVIQGYRTPVLSAEASTGETPNLGEVVVAEVGWDALKLNWTAPEGAYEYFFIQVQE |
| | ADTVEAAQNLTVPGGLRSTDLPGLKAATHYTITTRGVTQDFSTTPLSVEVLTEEVPDMGN |
| | LTVTEVSWDALRLNWTTPDGTYDQFTIQVQEADQVEEAHNLTVPGSLRSNEIPGLRAGTP |
| | YTVTLHGEVRGHSTRPLAVEVVTEDLPQLGDLAVSEVGWDGLRLNWTAADNAYEHFVIQV |
| | QEVNKVEAAQNLTLPGSLRAVDTPGLEAATPYRVSIYGVIRGYRTPVLSAEASTAKEPEI |
| | GNLNVSDITPESFNLSWMATDGIFETFTIEIIDSNRLLETVEYNISGAERTAHISGLPPS |
| | TDFIVYLSGLAPSIRTKTISATATTEALPLLENLTISDINPYGFTVSWMASENAFDSFLV |
| | TVVDSGKLLDPQEFTLSGTQRKLELRGLITGIGYEVMVSCFTQGHQTKPLRAEIVTEAEP |
| | EVDNLLVSDATPDGFRLSWTADEGVFDNFVLKIRDTKKQSEPLEITLLAPERTRDITGLR |
| | EATEYEIELYGISKGRRSQTVSAIATTANGSPKEVIFSDITENSATVSWRAPTAQVESFR |
| | ITYVPITGGTPSMVTVDGTKTQTRLVKLIPGVEYLVSIIAMKGFEESEPVSGSFTTALDG |
| | PSGLVTANITDSEALARWQPAIATVDSYVISYTGEKVPEITRTVSGNTVEYALTDLEPAT |
| | EYTLRIFAEKGPQKSSTITAKFTTDLDSPRDLTATEVQSETALLTWRPPRASVTGYLLVY |
| | ESVDGTVKEVIVGPDTTSYSLADLSPSTHYTAKIQALNGPLRSNMTQTIFTTIGLLYPFP |
| | KDCSQAMLNGDTTSGLYTIYLNGDKAQALEVFCDMTSDGGGWIVFLRRKNGRENFYQNWK |
| | AYAAGFGDRREEFWLGLDNLNKITAQGQYELRVDLRDHGETAFAVYDKFSVGDAKTRYKL |
| | KVEGYSGTAGDSMAYHNGRSFSTFDKDTDSAITNCALSTRGFWYRNCHRVNLMGRYGDNN |
| | HSQGVNWFHWKGHEHSIQFAEMKLRPSNFRNLEGRRKRA |

| | |
|---|---|
| SEQ ID NO: 183 | 7013 bp |

| | |
|---|---|
| NOV35b, | GAATTCGCTAGAGCCCTAGAGCCCCAGCACCACCCAGCCAAACCCACCTCCACCATGGGG |
| CG655832-03 | |
| DNA Sequence | GCCATGACTCAGCTGTTGGGACGTGTCTTTCTTGCTTTCCTTGCCCTCGCTACCGAAGGT |
| | GGGGTCCTCAAGAAAGTCATCCGGCACAAGCGACAGACTGGGGTGAACGCCACCCTGCCA |
| | GAAGAGAACCAGCCAGTGGTGTTTAACCACGTTTACAACATCAACCTCCCAGTGCGATCC |
| | CAGTGTTCGGTGGATCTGGAGTCAGCCAGTCGGGAGAAAGACCTGGCACCGCCTTCAGAG |
| | CCCAGCGAAAGCTTTCAGGAGCACACAGTACATGGGGAAAACCAGATTGTCTTCACACAT |
| | CGCATCAACATCCCCCGCCGGGCCTGTGCCTGTGCCGCAGCCCCTGATGTTAAGGAGCTG |
| | CTGAGCAGACTGGAGGAGCTGGAGAACCTGGTGTCTTCCCTGAGGGAGCAATGTACTGCA |
| | GGAGCAGGCTGCTGTCTCCAGCCTGCCACAGGCCGCTTGGACACCAGGCCCTTCTGTAGC |
| | GGTCGGGGCAACTTCAGCACTGAAGGATGTGGCTGTGTCTGCGAACCTGGCTGGAAAGGC |
| | CCCAACTGCTCTGAGCCCGAATGTCCAGGCAACTGTCACCTTCGAGGCCGGTGCATTGAT |
| | GGGCAGTGCATCTGTGACGACCGCTTCACGGGCGAGGACTGCAGCCAGCTGGCTTGCCCC |
| | AGCGACTCCAATGACCAGGGCAAGTGCGTGAATGGAGTCTGCATCTGTTTCGAAGGCTAC |
| | GCGGCTGACTGCAGCCGTGAAATCTGCCCAGTGCCCTGCAGTGAGGAGCACGGCACATGT |
| | GTAGATGGCTTGTGTGTGTGCCACCATGGCTTTGCAGCCGATGACTGCAACAAGCCTCTG |
| | TGTCTCAACAATTGCTACAACCGTGGACGATGCGTGCAGAATGAGTGCGTGTGTGATGAG |
| | GGTTTCACGGGCGAAGACTGCAGTGAGCTCATCTGCCCCAATGACTGCTTCGACCGGGGC |
| | CGCTGCATCAATGGCACCTGCTACTGCGAAGAAGGCTTCACAGGTGAAGACTGCGGGAAA |
| | CCCACCTGCCCACATGCCTGCCACACCCAGGGCCGGTGTGAGGAGGCGCAGTGTGTATGT |
| | GATGAGGGCTTTGCCGGTGTGGACTGCAGCGAGAAGAGGTGTCCTGCTGACTGTCACAAT |
| | CGTGGCCGCTGTGTAGACGGGCGGTGTGAGTGTGATGATGGTTTCACTGGAGCTGACTGT |
| | GGGGAGCTCAAGTGTCCCAATGGCTGCAGTGCCCATGGCCGCTGTGTCAATGGGCAGTGT |
| | GTGTGTGATGAGGGCTATACTGGGGAGGACTGCAGCCAGCTACGGTGCCCCAATGACTGT |
| | CACAGTCGGGGCCGCTGTGTCGAGGCCAAATGTGTATGTGAGCAAGGCTTCAAGGGCTAT |
| | GACTGCAGTGACATGAGCTGCCCTAATGACTGTCACCAGCACGGCCGCTGTGTGAATGGC |
| | ATGTGTGTTTCTGATGACGGCTACACAGGCGAAGACTGCCGGGATCGCCAATGCCCCAGG |
| | GACTGCAGCAACAGGGGCCTCTGTGTGGACGGACAGTGCGTCTGTGAGGACGGCTTCACC |
| | GGCCCTGACTGTGCAGAACTCTCCTGTCCAAATGACTGCCATGGCCAGGGTCGCTGTGTG |
| | AATGGGCAGTGCGTGTGCCATGAAGGATTTATGGGCAAAGACTGCAAGGAGCAAAGATGT |
| | CCCAGTCACTGTCATGGCCAGGGCCGCTGCGTGCACGGCCAGTGCATCTGCCACGAGGGC |
| | TTCACAGGCCTGGACTGTGGCCAGCACTCCTCCCCCAGTGACTGCAACAACTTAGGACAA |
| | TGCGTCTCGGGCCGCTGCATCTGCAACGAGCGCTACAGCGGAGAAGACTGCTCAGAGGTG |
| | TCTCCTCCCAAACACCTCGTTGTGACAGAAGTGACGGAAGAGACGGTCAACCTGGCCTGG |
| | GACAATGAGATGCGGCTCACAGAGTACCTTGTCGTGTACACGCCCACCCACGACCGTGGT |
| | CTGGAAATGCAGTTCCGTGTGCCTGGGGACCAGACGTCCACCATCATCCGGGAGCTGGAG |
| | CCTGGTCTCGAGTACTTTATCCGTGTATTTGCCATCCTCGAGAACAAGAAGAGCATTCCT |
| | GTCAGCGCCAGGGTGGCCACGTACTTACCTGCACCTGAAGGCCTGAAATTCAAGTCCATC |
| | AAGGAGACATCTGTGGAAGTGGAGTGGGATCCTCTAGACATTCCTTTTGAAACCTGGGAG |
| | ATCATCTTCCCGAATATGAATAAAGAAGATGAGGGAGAGATCACCAAAAGCCTGAGGAGG |
| | CCAGAGACCTCTTACCGGCAAACTGGTCTAGCTCCTGGGCAAGAGTATGAGATATCTCTG |
| | CACATAGTGAAAAACAATACCCGGGCCCCTGGCCTGAAGAGGGTGACCACCACACGCTTG |
| | GATGCCCCCAGCCAGATCGAGGTGAAAGATGTCACAGACACCACTGCCTTCATCACCTGG |
| | TTCAAGCCCCTGGCTGAGATCGATGGCATTGACCTGACCTACGGCATCAAAGACGTGCCA |
| | GGAGACCGTACCACCATCGATCTCACAGAGGACGAGAACCAGTACTCCATCGGGAACCTG |
| | AAGCCTGACACTGAGTACGAGGTGTCCCTCATCTCCCGCACACGTGACATGTCAAGCAAC |
| | CCAGCCAAAGAGACCTTCACAACAGGCCTCGATGCTCCCAGGAATCTTCGACGTGTTTCC |
| | CAGACAGATAACAGCATCACCCTGCAATGGAGGAATGGCAAGCCAGCTATTGACAGTTAC |
| | AGAATTAAGTATGCCCCCATCTCTCGAGGGGACCACCCTGAGGTTGATCTTCCAAAGAGC |
| | CAACAAGCCACAACCAAAACCACACTCACAGGTCTGAGGCCGCGAACTGAATATGGGATT |
| | GGAGTTTCTGCTGTGAAGCAAGACAAGGAGAGCAATCCAGCGACCATCAACGCAGCCACA |
| | GAGTTGGACACGCCCAAGGACCTTCAGGTTTCTGAAACTGCACAGACCAGCCTGACCCTG |
| | CTCTGGAAGACACCGTTGGCCAAATTTGACCGCTACCGCCTCAATTACAGTCTCCCCACA |
| | GGCCAGTGGGTGGGAGTGCAGCTTCCAAGAAACACCACTTCCTATCTCCTGAGACGCCTG |
| | GAACCAGGACAGGAGTACAATGTCCTCCTGACAGCCCAGACAGGCAGACACAAGAGCAAG |
| | CCCGCACGTGTGAAGGCATCCACTGAACAAGCCCCTGAGCTGGAAAACCTCACCCTGACT |
| | GAGGTTGGCTGGGATGGCCTCAGACTCAACTGGACCGCGGCTGACCAGCCCTATGAGCAC |
| | TTTATCATTCAGGTGCAGGAGGCCAACAACGTGGAGGCAGCTCGGAACCTCACCGTGCCT |
| | GGCAGCCTTCGGGCTGTGGACATACCGGGCCTCAAGCCTCCTACCCCTTATACAGTCTCC |
| | ATCTATGGGGTGATCCAGGGCTATACAACACCAGTGCTCTCTGCTGAGGCCTCCACAGGG |
| | GAAACTCCCAATTTGGGAGACGTCGTCCTGCCCGAGGTGCGCTGGGATGCCCTCAAACTC |
| | AACTGGACTGCTCCAGAAGGGGCCTATGAGTACTTTTTCATTCAGGTGCACGAGGCTGAC |
| | ACAGTAGAGGCAGCCCAGAACCTCACCGTCCCAGGAGGACTCAGGTCCACAGACCTGCCT |
| | GGGCTCAAAGCACCCACTCATTATACCATCACCATCCGCGGGGTCACTCAGGACTTCAGC |
| | ACAACCCCTCTCTCTGTTGAAGTCTTGACAGAGGAGGTTCCAGATATGGGAAACCTCACA |
| | GTGACCGAGGTTAGCTGGGATGCTCTCAGACTGAACTGGACCACGCCAGATGGAACCTAT |
| | GACCAGTTTACTATTCAGGTCCAGGAGGCTGACCACGTGGAAGAGGCTCACAATCTCACG |
| | GTTCCTGGCAGCCTGCGTTCCATGGAAATCCCAGGCCTCAGCCCTGGCACTCCTTACACA |
| | GTCACCCTGCACGGCGAGGTCAGGCGCCACAGCACTCGACCCCTTGCTGTAGACCTCGTC |
| | ACAGAGGATCTCCCACAGCTGGCAGATTTAGCCGTGTCTGAGGTTGCCTGGGATGGCCTC |
| | AGACTCAACTGGACCGCAGCTGACAATGCCTATGAGCACTTTGTCATTCAGGTGCAGGAG |
| | GTCAACAAAGTGGAGGCAGCCCAGAACCTCACGTTGCCTCGCAGCCTCAGGGCTGTGGAC |
| | ATCCCGGGCCTCGAGGCTGCCACGCCTTATAGAGTCTCCATCTATGGGGTGATCCGGGGC |
| | TATAGAACACCAGTACTCTCTGCTGAGCCCTCCACAGCCAAAGAACCTGAAATTGGAAAC |
| | TTAAATGTTTCTGACATAACTCCCGAGAGCTTCAATCTCTCCTGGATGGCTACCGATGGG |
| | ATCTTCGAGACCTTTACCATTGAAATTATTGATTCCAATAGGTTGCTGGAGACTGTGGAA |
| | TATAATATCTCTGGTGCTGAACGAACTGCCCATATCTCAGOGCTACCCCCTAGTACTGAT |
| | TTTATTGTCTACCTCTCTGGACTTGCTCCCAGCATCCGGACCAAAACCATCAGTGCCACA |
| | GCCACGACAGAAGCCGAACCGGAAGTTGACAACCTTCTGGTTTCAGATGCCACCCCAGAC |
| | GGTTTCCGTCTGTCCTGGACAGCTGATGAAGGGGTCTTCGACAATTTTGTTCTCAAAATC |
| | AGAGATACCAAAAAGCAGTCTGAGCCACTGGAAATAACCCTACTTGCCCCCGAACGTACC |
| | AGGGACATAACAGGTCTCAGAGAGGCTACTGAATACGAAATTGAACTCTATGGAATAAGC |
| | AAAGGAAGGCGATCCCAGACAGTCAGTGCTATACCAACAACACCCATGGGCTCCCCAAAG |
| | GAAGTCATTTTCTCAGACATCACTGAAAATTCGGCTACTGTCAGCTGGAGGGCACCCACG |
| | GCCCAAGTGGAGAGCTTCCGGATTACCTATGTGCCCATTACAGGAGGTACACCCTCCATG |
| | GTAACTGTGGACGGAACCAAGACTCAGACCAGGCTGGTGAAACTCATACCTGCCGTGGAG |
| | TACCTTGTCAGCATCATCGCCATGAAGGGCTTTGAGGAAAGTGAACCTGTCTCAGGGTCA |
| | TTCACCACACCTCTGGATGGCCCATCTGGCCTGGTGACAGCCAACATCACTGACTCAGAA |
| | GCCTTGGCCAGGTGCCAGCCAGCCATTGCCACTGTGGACAGTTATGTCATCTCCTACACA |
| | GGCGAGAAAGTGCCAGAAATTACACGCACGGTGTCCGGGAACACAGTGGAGTATGCTCTG |
| | ACCGACCTCGAGCCTGCCACGGAATACACACTGAGAATCTTTGCAGAGAAAGGGCCCCAG |
| | AAGAGCTCAACCATCACTGCCAAGTTCACAACAGACCTCGATTCTCCAAGAGACTTGACT |
| | GCTACTGAGGTTCAGTCGGAAACTGCCCTCCTTACCTGGCGACCCCCCCGGGCATCAGTC |
| | ACCGGTTACCTGCTGCTCTATGAATCAGTGGATGGCACAGTCAAGGAAGTCATTGTGGGT |
| | CCAGATACCACCTCCTACAGCCTGGCAGACCTGACCCCATCCACCCACTACACAGCCAAG |
| | ATCCAGGCACTCAATGGGCCCCTGAGGAGCAATATGATCCAGACCATCTTCACCACAATT |
| | GGACTCCTGTACCCCTTCCCCAAGGACTGCTCCCAAGCAATGCTGAATGCAGACACCACC |
| | TCTGGCCTCTACACCATTTATCTGAATCGTGATAAGGCTCAGGCGCTGGAAGTCTTCTGT |
| | GACATGACCTCTGATGGGGGTGGATGGATTGTGTTCCTGAGACGCAAAAACGGACGCGAG |
| | AACTTCTACCAAAACTGGAAGGCATATGCTGCTGGATTTGGGGACCGCAGAGAAGAATTC |
| | TGGCTTGGGCTGGACAACCTGAACAAAATCACAGCCCAGGGGCAGTACGAGCTCCGGGTG |
| | GACCTGCGGGACCATGGCGAGACACCCTTTGCTGTCTATGACAAGTTCAGCGTGGGAGAT |
| | GCCAAGACTCGCTACAAGCTGAAGGTGGAGGGCTACAGTCGGACAGCACGTGACTCCATG |
| | GCCTACCACAATGGCAGATCCTTCTCCACCTTTGACAAGGACACAGATTCAGCCATCACC |
| | AACTGTGCTCTGTCTACAAGGGGCTTCTGGTACAGGAACTGTCACCGTGTCAACCTGATG |
| | GGGAGATATGGGGACAATAACCACAGTCAGGGCGTTAACTGGTTCCACTGGAAGGGCCAC |
| | GAACACTCAATCCAGTTTGCTGAGATGAAGCTGAGACCAAGCAACTTCAGAAATCTTGAA |
| | GGCAGGCGCAAACGGGCATAAATTGGAGGGACCACTGGGTGAGAGAGGAATAAGGCGGCC |
| | CAGAGCGAGGAAAGGATTTTACCAAAGCATCAATACAACCAGCCCAACCATCGGTCCACA |
| | CCTGGGCATTTGGTGAGAATCAAAGCTGACCATGGATCCCTGCGGCCAACGGCAACAGCA |
| | TGGGCCTCACCTCCTCTCTGATTTCTTTCTTTGCACCAAAGACATCAGTCTCCAACATGT |
| | TTCTGTTTTGTTGTTTGATTCAGCAAAAATCTCCCAGTGACAACATCGCAATAGTTTTTT |
| | ACTTCTCTTAGGTGGCTCTGGGATGGGAGAGGGGTAGGATGTACAGGGGTAGTTTGTTTT |
| | AGAACCAGCCGTATTTTACATGAAGCTGTATAATTAATTGTCATTATTTTTGTTAGCAAA |
| | GATTAAATGTGTCATTGGAAGCCATCCCTTTTTTTACATTTCATACAACAGAAACCAGAA |
| | AAGCAATACTGTTTCCATTTTAAGCATATGATTAATATTATTAATATAATAATGATGATG |
| | ATGATGATGAAAACTAAGGATTTTTCAAGAGATCTTTCTTTCCAAAACATTTCTGGACAG |
| | TACCTGATTGTATTTTTTTTTTAAATAAAAGCACAAGTACTTTTGAAAAAAAA |

| | | |
|---|---|---|
| ORF Start: ATG at 55 | | ORF Stop: TAA at 6379 |
| SEQ ID NO: 184 | 2108 aa | MW at 230729.3 kD |

| | |
|---|---|
| NOV35b | MGAMTQLLAGVFLAFLALATEGGVLKKVIRHKRQSGVNATLPEENQPVVFNHVYNIKLPV |
| CG55832-03 | |
| Protein Sequence | GSQCSVDLESASGEKDLAPPSEPSESFQEHTVDGENQIVFTHRINIPRRACCCAAAPDVK |
| | ELLSRLEELENLVSSLREQCTACAGCCLQPATGRLDTRPFCSCRGNFSTECCGCVCEPGW |
| | KGPNCSEPECPGNCHLRGRCIDCQCICDDGFTGEDCSQLACPSDCNDQGKCVNGVCTCFE |
| | GYAADCSREICPVPCSEEHGTCVDGLCVCHDGFAGDDCNKPLCLNNCYNRGRCVENECVC |
| | DEGFTGEDCSELICPNDCFDRGRCINGTCYCEEGFTGEDCGKPTCPHACNTQGRCEEGQC |
| | VCDEGFAGVDCSEKRCPADCHNRGRCVDGRCECDDGFTGADCGELKCPNCCSGHGRCVNG |
| | QCVCDEGYTGEDCSQLRCPNDCHSRGRCVEGKCVCEQGFKGYDCSDMSCPNDCHQHGRCV |
| | NGMCVCDDGYTGEDCRDRQCPRDCSNRGLCVDGQCVCEDGFTGPDCAELSCPNDCHGQGR |
| | CVNGQCVCHEGFMGKDCKEQRCPSDCHGQGRCVDGQCICHEGFTGLDCGQHSCPSDCNNL |
| | GQCVSGRCICNEGYSGEDCSEVSPPKDLVVTEVTEETVNLAWDNEMRVTEYLVVYTPTHE |
| | GGLEMQFRVPGDQTSTIIRELEPGVEYFIRVFAILENKKSIPVSARVATYLPAPEGLKFK |
| | SIKETSVEVEWDPLDIAFETWEIIFRNMNKEDEGEITKSLRRPETSYRQTGLAPGQEYEI |
| | SLHIVKNNTRGPGLKRVTTTRLDAPSQIEVKDVTDTTALITWFKPLAEIDGIELTYGIKD |
| | VPGDRTTIDLTEDENQYSIGNLKPDTEYEVSLISRRGDMSSNPAKETFTTGLDAPRNLRR |
| | VSQTDNSITLEWRNGKAAIDSYRIKYAPISGGDHAEVDVPKSQQATTKTTLTGLRPGTEY |
| | GIGVSAVKEDKESNPATINAATELDTPKDLQVSETAETSLTLLWKTPLAKFDRYRLNYSL |
| | PTGQWVGVQLPRNTTSYVLRGLEPGQEYNVLLTAEKGRHKSKPARVKASTEQAPELENLT |
| | VTEVGWDGLRLNWTAADQAYEHFIIQVQEANKVEAARNLTVPGSLRAVDIPGLKAATPYT |
| | VSIYGVIQGYRTPVLSAEASTGETPNLGEVVVAEVGWDALKLNWTAPEGAYEYFFIQVQE |
| | ADTVEAAQNLTVPGGLRSTDLPGLKAATHYTITIRGVTQDFSTTPLSVEVLTEEVPDMGN |
| | LTVTEVSWDALRLNWTTPDGTYDQFTIQVQEADQVEEAHNLTVPGSLRSMEIPGLRAGTP |
| | YTVTLHGEVRGHSTRPLAVEVVTEDLPQLGDLAVSEVGWDGLRLNWTAADNAYEHFVIQV |
| | QEVNKVEAAQNLTLPGSLRAVDIPGLEAATPYRVSIYGVIRGYRTPVLSAEASTAKEPEI |
| | GNLNVSDITPESFNLSWMATDGIFETFTIEIIDSNRLLETVEYNISGAERTAHISGLPPS |
| | TDFIVYLSGLAPSIRTKTISATATTEAEPEVDNLLVSDATPDGFRLSWTADECVFDNFVL |
| | KIRDTKKQSEPLEITLLAPERTRDITCLREATEYEIELYGISKGRRSQTVSAIATTAMGS |
| | PKEVIFSDITENSATVSWRAPTAQVESFRITYVPITGGTPSMVTVDGTKTQTRLVKLIPG |
| | VEYLVSIIAMKGFEESEPVSGSFTTALDGPSGLVTANITDSEALARWQPAIATVDSYVIS |
| | YTGEKVPEITRTVSGNTVEYALTDLEPATEYTLRIFAEKGPQKSSTITAKFTTDLDSPRD |
| | LTATEVQSETALLTWRPPRASVTGYLLVYESVDGTVKEVIVGPDTTSYSLADLSPSTHYT |
| | AKIQALNGPLRSNMIQTIFTTIGLLYPFPKDCSQAMLNGDTTSGLYTIYLNGDKAQALEV |
| | FCDMTSDGGGWIVFLRRKNGRENFYQNWKAYAAGFGDRREEFWLGLDNLNKITAQGQYEL |
| | RVDLRDHGETAFAVYDKFSVGDAKTRYKLKVEGYSGTAGDSMAYHNGRSFSTFDKDTDSA |
| | ITNCALSTRGFWYRNCHRVNLMGRYGDNNHSQGVNWFHWKGHEHSIQFAEMKLRPSNFRN |
| | LEGRRKRA |

| | |
|---|---|
| SEQ ID NO: 185 | 5375 bp |

| | |
|---|---|
| NOV35c, | GAATTCGCTAGAGCCCTAGAGCCCCAGCAGCACCCAGCCAAACCCACCTCCACCATGGGG |
| CG55832-02 | |
| DNA Sequence | GCCATGACTCAGCTGTTGGCAGGTGTCTTTCTTGCTTTCCTPGCCCTCGCTACCGAAGGT |
| | GGGGTCCTCAAGAAAGTCATCCGGCACAAGCGACACAGTGGGGTGAACGCCACCCTGCCA |
| | GAAGAGAACCAGCCAGTGGTGTTTAACCACGTTTACAACATCAAGCTGCCACTGGGATCC |
| | CAGTGTTCGGTGGATCTGGAGTCAGCCAGTGGGGACAAACACCTCGCACCGCCTTCAGAG |
| | CCCAGCGAAAGCTTTCAGGAGCACACAGTAGATGGGGAAAACCAGATTGTCTTCACACAT |
| | CGCATCAACATCCCCCGCCGGGCCTGTGCCTGTGCCGCAGCCCCTCATGTTAAGGAGCTG |
| | CTGAGCAGACTGGAGGAGCTGGAGAACCTGGTGTCTTCCCTGAGGCAGCAATGTACTGCA |
| | GGAGCACGCTGCTGTCTCCAGCCTGCCACAGGCCGCTTGGACACCAGGCCCTTCTGTAGC |
| | GGTCGGGGCAACTTCAGCACTGAAGGATGTGGCTGTGTCTGCGAACCTGGCTGGAAAGGC |
| | CCCAACTGCTCTGAGCCCGAATGTCCAGGCAACTGTCACCTTCGAGGCCGGTGCATTGAT |
| | GGGCAGTGCATCTGTGACGACGGCTTCACCGGGCAGGACTGCAGCCAGCTGGCTTGCCCC |
| | AGCGACTGCAATGACCAGGGCAAGTGCGTGAATGGAGTCTCCATCTGTTTCGAAGGCTAC |
| | GCCGCTGACTGCAGCCGTGAAATCTGCCCAGTGCCCTGCAGTGACGAGCACGGCACATGT |
| | GTAGATGGCTTGTGTGTGTGCCACGATGGCTTTGCAGGCCATGACTGCAACAAGCCTCTG |
| | TGTCTCAACAATTGCTACAACCGTGGACGATGCGTGCAGAATGAGTGCCTGTCTGATCAG |
| | GGTTTCACGGGCGAAGACTGCAGTGAGCTCATCTCCCCCAATGACTGCTTCGACCCGGGC |
| | CGCTGCATCAATGGCACCTGCTACTGCGAAGAAGGCTTCACAGGTGAACACTGCGGGAAA |
| | CCCACCTGCCCACATGCCTGCCACACCCAGGCCCGGTGTGAGGAGGGGCAGTGTGTATGT |
| | GATGAGGGCTTTGCCGGTGTGGACTGCAGCGAGAAGAGGTGTCCTGCTGACTGTCACAAT |
| | CGTGGCCGCTGTGTACACGGGCGCTGTGAGTGTGATCATGGTTTCACTGGAGCTGACTGT |
| | GGGGAGCTCAAGTGTCCCAATGCCTGCAGTGGCCATGGCCGCTGTGTCAATGGGCAGTGT |
| | GTGTGTGATGAGGGCTATACTGGGGACGACTGCACCCAGCTACGGTGCCCCAATGACTGT |
| | CACAGTCGGCGCCCCTGTGTCGAGGGCAAATCTCTATGTCAGCAAGGCTTCAAGGGCTAT |
| | GACTGCAGTCACATGAGCTGCCCTAATGACTGTCACCAGCACGGCCGCTGTGTGAATGGC |
| | ATGTGTGTTTGTGATGACGGCTACACAGGGGAAGACTGCCGGGATCGCCAATGCCCCAGG |
| | GACTGCAGCAACAGGGCCCTCTGTGTCGACGGACAGTGCGTCTGTGAGGACGGCTTCACC |
| | GGCCCTGACTGTGCAGAACTCTCCTGTCCAAATCACTGCCATGGCCAGGGTCGCTGTGTG |
| | AATGGGCAGTGCGTGTGCCATGAAGGATTTATGGGCAAAGACTGCAAGGAGCAAAGATGT |
| | CCCAGTGACTGTCATGGCCAGGGCCGCTGCGTGGACGGCCAGTGCATCTGCCACGAGGGC |
| | TTCACAGGCCTGGACTGTGGCCAGCACTCCTCCCCCAGTGACTGCAACAACTTAGGACAA |
| | TGCGTCTCGGGCCGCTGCATCTGCAACGAGGGCTACAGCGGAGAAGACTGCTCAGAGGTG |
| | TCTCCTCCCAAAGACCTCGTTGTGACAGAAGTGACGGAAGAGACGGTCAACCTGGCCTGG |
| | GACAATGAGATGCGGGTCACAGAGTACCTTGTCGTGTACACGCCCACCCACCAGGGTGGT |
| | CTGGAAATGCAGTTCCGTGTGCCTGGGGACCAGACGTCCACCATCATCCGGGAGCTGGAG |
| | CCTGGTGTGGAGTACTTTATCCGTGTATTTGCCATCCTGGAGAACAAGAAGAGCATTCCT |
| | GTCAGCGCCAGGGTGGCCACGTACTTACCTGCACCTGAAGGCCTGAAATTCAAGTCCATC |
| | AAGCAGACATCTGTGGAAGTGGAGTGGGATCCTCTAGACATTGCTTTTGAAACCTGGGAG |
| | ATCATCTTCCGCAATATGAATAAAGAAGATGAGGGAGACATCACCAAAAGCCTGAGGAGG |
| | CCAGAGACCTCTTACCGGCAAACTGGTCTAGCTCCTGGGCAAGAGTATGAGATATCTCTG |
| | CACATAGTGAAAAACAATACCCGGGGCCCTGGCCTGAAGAGGGTGACCACCACACGCTTG |
| | GATGCCCCCAGCCAGATCGAGGTGAAAGATGTCACAGACACCACTGCCTTGATCACCTGG |
| | TTCAAGCCCCTGGCTGAGATCGATGGCATTGAGCTGACCTACGGCATCAAAGACGTGCCA |
| | GGAGACCGTACCACCATCCATCTCACAGAGGACGAGAACCAGTACTCCATCGGGAACCTG |
| | AAGCCTGACACTGAGTACGAGGTGTCCCTCATCTCCCGCAGAGGTGACATGTCAAGCAAC |
| | CCAGCCAAAGAGACCTTCACAACAGGCCTCGATGCTCCCAGGAATCTTCGACGTGTTTCC |
| | CAGACAGATAACAGCATCACCCTGGAATGCAGGAATGGCAAGGCAGCTATTGACAGTTAC |
| | AGAATTAAGTATGCCCCCATCTCTGGAGGGGACCACCCTGAGGTTGATGTTCCAAAGAGC |
| | CAACAAGCCACAACCAAAACCACACTCACAGGTCTGAGGCCGGGAACTGAATATGGGATT |
| | GGAGTTTCTCCTGTGAAGGAAGACAAGGAGAGCAATCCAGCGACCATCAACGCAGCCACA |
| | GAGTTGGACACGCCCAACGACCTTCAGGTTTCTGAAACTGCAGAGACCAGCCTGACCCTG |
| | CTCTGGAAGACACCGTTGGCCAAATTTGACCGCTACCGCCTCAATTACAGTCTCCCCACA |
| | GGCCAGTGGGTGGGACTGCAGCTTCCAAGAAACACCACTTCCTATGTCCTGAGAGGCCTG |
| | GAACCACGACAGGAGTACAATGTCCTCCTCACAGCCGACAAAGGCAGACACAACAGCAAG |
| | CCCGCACGTGTGAAGGCATCCACTCCCATGGCCTCCCCAAAGGAAGTCATTTTCTCAGAC |
| | ATCACTGAAAATTCGGCTACTGTCAGCTGGAGGCCACCCACAGCCCAAGTCGAGACCTTC |
| | CGGATTACCTATGTGCCCATTACACGAGGTACACCCTCCATGGTAACTGTGGACGGAACC |
| | AAGACTCAGACCAGGCTGGTGAAACTCATACCTCGCGTGGAGTACCTTGTCAGCATCATC |
| | GCCATGAAGGGCTTTGACGAAAGTGAACCTGTCTCAGQGTCATTCACCACAGCTCTGGAT |
| | GGCCCATCTGGCCTGGTGACAGCCAACATCACTGACTCAGAAGCCTTGGCCAGGTCGCAG |
| | CCAGCCATTGCCACTGTCGACAGTTATGTCATCTCCTACACACGCGAGAAAGTGCCAGAA |
| | ATTACACGCACGGTGTCCGGGAACACAGTGGAGTATGCTCTGACCGACCTCGAGCCTGCC |
| | ACGGAATACACACTGAGAATCTTTGCAGAGAAAGGGCCCCAGAAGAGCTCAACCATCACT |
| | GCCAAGTTCACAACACACCTCGATTCTCCAAGAGACTTGACTGCTACTGAGGTTCAGTCG |
| | GAAACTGCCCTCCTTACCTGGCGACCCCCCCGGGCATCAGTCACCGGTTACCTCCTGGTC |
| | TATGAATCAGTGGATGGCACAGTCAAGGAAGTCATTGTGGGTCCAGATACCACCTCCTAC |
| | AGCCTGGCAGACCTGAGCCCATCCACCCACTACACAGCCAAGATCCAGGCACTCAATGGG |
| | CCCCTGAGGAGCAATATGATCCAGACCATCTTCACCACAATTGGACTCCTGTACCCCTTC |
| | CCCAAGGACTGCTCCCAAGCAATGCTGAATGGAGACACGACCTCTGGCCTCTACACCATT |
| | TATCTGAATGCTGATAAGGCTCAGGCGCTGGAAGTCTTCTGTGACATGACCTCTGATGGG |
| | GGTGGATGGATTGTGTTCCTGAGACGCAAAAACCGACGCGAGAACTTCTACCAAAACTGG |
| | AAGGCATATGCTGCTGGATTTGGGGACCGCAGAGAAGAATTCTGGCTTGGGCTGGACAAC |
| | CTGAACAAAATCACAGCCCAGGGGCAGTACGAGCTCCGGGTGGACCTGCGGGACCATGGG |
| | GAGACACCCTTTGCTGTCTATGACAAGTTCAGCGTCGGAGATGCCAAGACTCGCTACAAG |
| | CTGAAGGTGGAGCGGTACAGTGGGACAGCAGGTGACTCCATGCCCTACCACAATGGCAGA |
| | TCCTTCTCCACCTTTGACAAGGACACAGATTCAGCCATCACCAACTGTCCTCTGTCTACA |
| | AGGGGCTTCTGGTACAGGAACTGTCACCGTGTCAACCTGATGGGGAGATATGGCGACAAT |
| | AACCACAGTCAGGGCGTTAACTGGTTCCACTGGAAGGGCCACGAACACTCAATCCAGTTT |
| | GCTGAGATGAAGCTGAGACCAAGCAACTTCAGAAATCTTGAAGGCAGGCCCAAACGGGCA |
| | TAAATTGGAGGGACCACTGCGTGAGAGAGGAATAAGGCGGCCCAGAGCGAGCAAACGATT |
| | TTACCAAACCATCAATACAACCACCCCAACCATCGGTCCACACCTGGGCATTTGGTGAGA |
| | ATCAAAGCTGACCATGGATCCCTGGGCCCAACGGCAACAGCATGGGCCTCACCTCCTCTG |
| | TGATTTCTTTCTTTGCACCAAAGACATCAGTCTCCAACATGTTTCTGTTTTGTTGTTTGA |
| | TTCAGCAAAAATCTCCCAGTGACAACATCCCAATAGTTTTTTACTTCTCTTAGGTGCCTC |
| | TGGGATGGGAGAGGGGTAGGATGTACAGGGGTAGTTTGTTTTAGAACCAGCCGTATTTTA |
| | CATGAAGCTGTATAATTAATTGTCATTATTTTTGTTAGCAAAGATTAAATGTGTCATTGG |
| | AAGCCATCCCTTTTTTTACATTTCATACAACAGAAACCAGAAAAGCAATACTGTTTCCAT |
| | TTTAAGGATATGATTAATATTATTAATATAATAATGATGATGATGATGATGAAAACTAAG |
| | GATTTTTCAAGAGATCTTTCTTTCCAAAACATTTCTGGACAGTACCTGATTGTATTTTTT |
| | TTTTAAATAAAAGCACAAGTACTTTTGAAAAAAAA |

| | | |
|---|---|---|
| ORF Start: ATG at 55 | | ORF Stop: TAA at 4741 |
| SEQ ID NO: 186 | 1562 aa | MW at 171222.6 kD |

| | |
|---|---|
| NOV35c, | MGAMTQLLAGVFLAFLALATEGGVLKKVIRHKRQSGVNATLPEENQPVVFNHVYNIKLPV |
| CG55832-02 | |
| Protein Sequence | GSQCSVDLESASGEKDLAPPSEPSESFQEHTVDGENQIVFTXRINIPRRACGCAAAPDVK |
| | ELLSRLEELENLVSSLREQCTAGAGCCLQPATGRLDTRPFCSGRGNFSTEGCGCVCEPGW |
| | KGPNCSEPECPGNCHLRGRCIDCQCICDDGFTGEDCSQLACPSDCNDQGKCVNGVCICFE |
| | GYAADCSREICPVPCSEEHGTCVDGLCVCHDGFAGDDCNKPLCLNNCYNRGRCVENECVC |
| | DEGFTGEDCSELICPNDCFDRGRCINGTCYCEEGFTGEDCGKPTCPHACMTQGRCEEGQC |
| | VCDEGFAGVDCSEKRCPADCHNRGRCVDGRCECDDGFTGADCGELKCPNGCSGHGRCVNG |
| | QCVCDEGYTGEDCSQLRCPNDCHSRGRCVEGKCVCEQGFKGYDCSDMSCPNDCHQHGRCV |
| | NGMCVCDDGYTGEDCRDRQCPRDCSNRGLCVDGQCVCEDGFTGPDCAELSCPNDCHGQGR |
| | CVNGQCVCHEGFMGKDCKEQRCPSDCHGQGRCVDGQCICHEGFTGLDCGQHSCPSDCNNL |
| | GQCVSGRCICNEGYSGEDCSEVSPPKDLVVTEVTEETVNLAWDNEMRVTEYLVVYTPTHE |
| | GGLEMQFRVPGDQTSTIIRELEPGVEYFIRVFAILENKKSIPVSARVATYLPAPEGLKFK |
| | SIKETSVEVEWDPLDIAFETWEIIFRNMNKEDEGEITKSLRRPETSYRQTGLAPGQEYEI |
| | SLHIVKNNTRGPGLKRVTTTRLDAPSQIEVKDVTDTTALITWFKPLAEIDGIELTYGIKD |
| | VPGDRTTIDLTEDENQYSIGNLKPDTEYEVSLISRRGDMSSNPAKETFTTGLDAPRNLRR |
| | VSQTDNSITLEWRNGKAAIDSYRIKYAPISGGDHAEVDVPKSQQATTKTTLTGLRPGTEY |
| | GIGVSAVKEDKESNPATINAATELDTPKDLQVSETAETSLTLLWKTPLAKFDRYRLNYSL |
| | PTGQWVGVQLPRNTTSYVLRGLEPGQEYNVLLTAEKGRHKSKPARVKASTAMGSPKEVIF |
| | SDITENSATVSWRAPTAQVESFRITYVPITGGTPSMVTVDGTKTQTRLVKLIPGVEYLVS |
| | IIAMKGFEESEPVSGSFTTALDCPSGLVTANITDSEALARWQPAIATVDSYVISYTGEKV |
| | PEITRTVSGNTVEYALTDLEPATEYTLRIFAEKGPQKSSTITAKFTTDLDSPRDLTATEV |
| | QSETALLTWRPPRASVTGYLLVYESVDGTVKEVIVGPDTTSYSLADLSPSTHYTAKIQAL |
| | NGPLRSNMIQTIFTTIGLLYPFPKDCSQAMLNGDTTSGLYTIYLNGDKAQALEVFCDMTS |
| | DGGGWIVFLRRKNGRENFYQNWKAYAAGFGDRREEFWLGLDNLNKITAQGQYELRVDLRD |
| | HGETAFAVYDKFSVGDAKTRYKLKVECYSGTAGDSMAYHNGRSFSTFDKDTDSAITNCAL |
| | STRGFWYRNCHRVNLMCRYGDNNHSQGVNWFHWKGHEHSIQFAEMKLRPSNFRNLEGRRK |
| | RA |

Sequence comparison of the above protein sequences yields the following sequence relationships shown in Table 35B.

TABLE 35B
Comparison of NOV35a against NOV35b and NOV35c.

| Protein | NOV35a Residues/ | Identities/Similarities |
|---|---|---|
| Sequence | Match Residues | for the Matched Region |
| NOV35b | 1 . . . 1884 | 1595/1886 (84%) |
| | 1 . . . 1881 | 1660/1886 (87%) |
| NOV35c | 1 . . . 1332 | 1079/1335 (80%) |
| | 1 . . . 1327 | 1120/1335 (83%) |

Twelve polymorphic variants of NOV35c have been identified and are shown in Table 41N.

Further analysis of the NOV35a protein yielded the following properties shown in Table 35C.

TABLE 35C
Protein Sequence Properties NOV35a

| | |
|---|---|
| PSort analysis: | 0.8200 probability located in endoplasmic reticulum |
| | (membrane); 0.1900 probability located in plasma |
| | membrane; 0.1000 probability located in endoplasmic |
| | reticulum (lumen); 0.1000 probability located |
| | in outside |
| SignalP analysis: | Cleavage site between residues 23 and 24 |

A search of the NOV35a protein against the Geneseq database, a proprietary database that contains sequences published in patents and patent publication, yielded several homologous proteins shown in Table 35D.

TABLE 35D
Geneseq Results for NOV35a

| | | NOV35a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAR94562 | Human cytotactin - Homo | 1 . . . 2199 | 2199/2199 (100%) | 0.0 |
| | sapiens, 2199 aa. | 1 . . . 2199 | 2199/2199 (100%) | |
| | [WO9608513-A1, 21 | | | |
| | MAR. 1996] | | | |
| AAB36935 | Human tenascin-C - Homo | 1 . . . 2199 | 2194/2201 (99%) | 0.0 |
| | sapiens, 2201 aa. | 1 . . . 2201 | 2198/2201 (99%) | |
| | [WO200066628-A1, 09 | | | |
| | NOV. 2000] | | | |
| AAR94563 | Chicken cytotactin - Gallus | 1 . . . 1602 | 848/1620 (52%) | 0.0 |
| | sp, 1810 aa. [WO9608513- | 1 . . . 1581 | 1121/1620 (68%) | |
| | A1, 21 MAR. 1996] | | | |
| AAM39043 | Human polypeptide SEQ ID | 627 . . . 2194 | 544/1741 (31%) | 0.0 |
| | NO 2188 - Homo sapiens, | 2901 . . . 4616 | 834/1741 (47%) | |
| | 4618 aa. [WO200153312- | | | |
| | A1, 26 JUL. 2001] | | | |
| AAW18824 | Human restrictin - Homo | 484 . . . 1414 | 338/935 (36%) | 0.0 |
| | sapiens, 1358 aa. | 188 . . . 1107 | 528/935 (56%) | |
| | [US5635360-A, 03 JUN. | | | |
| | 1997] | | | |

In a BLAST search of public sequence datbases, the NOV35a protein was found to have homology to the proteins shown in the BLASTP data in Table 35E.

TABLE 35E
Public BLASTP Results for NOV35a

| | | NOV35a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P24821 | Tenascin precursor (TN) | 1 . . . 2199 | 2194/2201 (99%) | 0.0 |
| | (Hexabrachion) (Cytotactin) | 1 . . . 2201 | 2198/2201 (99%) | |
| | (Neuronectin) (GMEM) (JI) | | | |
| | (Miotendinous antigen) | | | |
| | (Glioma-associated- | | | |
| | extracellular matrix antigen) | | | |
| | (GP 150-225) (Tenascin-C) | | | |
| | (TN-C) - Homo sapiens | | | |
| | (Human), 2201 aa. | | | |
| JQ1322 | tenascin precursor - mouse, | 1 . . . 1796 | 1282/1807 (70%) | 0.0 |
| | 2019 aa. | 1 . . . 1791 | 1453/1807 (79%) | |
| Q64706 | Tenascin C precursor - Mus | 1 . . . 1796 | 1277/1807 (70%) | 0.0 |
| | musculus (Mouse), 2019 aa. | 1 . . . 1791 | 1449/1807 (79%) | |
| Q29116 | Tenascin precursor (TN) | 1 . . . 1528 | 1050/1532 (68%) | 0.0 |
| | (Hexabrachion) (Cytotactin) | 1 . . . 1521 | 1213/1532 (78%) | |
| | (Neuronectin) (GMEM) (JI) | | | |
| | (Miotendinous antigen) | | | |
| | (Glioma-associated- | | | |
| | extracellular matrix antigen) | | | |
| | (GP 150-225) (Tenascin-C) | | | |
| | (TN-C) (P230) - Sus scrofa | | | |
| | (Pig), 1746 aa. | | | |
| P10039 | Tenascin precursor (TN) | 1 . . . 1602 | 849/1618 (52%) | 0.0 |
| | (Hexabrachion) (Cytotactin) | 1 . . . 1579 | 1123/1618 (68%) | |
| | (Neuronectin) (GMEM) (JI) | | | |
| | (Miotendinous antigen) | | | |
| | (Glioma-associated- | | | |
| | extracellular matrix antigen) | | | |
| | (GP 150-225) - Gallus gallus | | | |
| | (Chicken), 1808 aa. | | | |

PFam analysis predicts that the NOV35a protein contains the domains shown in Table 35F.

TABLE 35F
Domain Analysis of NOV35a

| Pfam | NOV35a | Identities/Similarities | Expect |
|---|---|---|---|
| Domain | Match Region | for the Matched Region | Value |
| EGF | 185 . . . 216 | 10/48 (21%) | 0.34 |
| | | 27/48 (56%) | |
| EGF | 251 . . . 278 | 13/47 (28%) | 0.51 |
| | | 24/47 (51%) | |
| EGF | 283 . . . 309 | 12/47 (26%) | 0.0055 |
| | | 22/47 (47%) | |
| EGF | 314 . . . 340 | 12/47 (26%) | 0.076 |
| | | 21/47 (45%) | |
| EGF | 345 . . . 371 | 9/47 (19%) | 0.93 |
| | | 20/47 (43%) | |
| EGF | 376 . . . 402 | 13/47 (28%) | 0.0026 |
| | | 22/47 (47%) | |
| EGF | 407 . . . 433 | 14/47 (30%) | 0.0014 |
| | | 25/47 (53%) | |
| EGF | 469 . . . 495 | 13/47 (28%) | 0.0049 |
| | | 22/47 (47%) | |
| EGF | 500 . . . 526 | 13/47 (28%) | 0.0023 |
| | | 22/47 (47%) | |
| EGF | 531 . . . 557 | 12/47 (26%) | 0.007 |
| | | 23/47 (49%) | |
| EGF | 562 . . . 588 | 11/47 (23%) | 0.0033 |
| | | 24/47 (51%) | |
| EGF | 593 . . . 619 | 12/47 (26%) | 0.023 |
| | | 24/47 (51%) | |
| fn3 | 622 . . . 700 | 29/85 (34%) | 5.5e−15 |
| | | 58/85 (68%) | |
| fn3 | 711 . . . 794 | 24/87 (28%) | 2.6e−13 |
| | | 65/87 (75%) | |
| fn3 | 802 . . . 881 | 26/85 (31%) | 1.9e−15 |
| | | 66/85 (78%) | |
| fn3 | 892 . . . 973 | 35/87 (40%) | 4.1e−19 |
| | | 65/87 (75%) | |
| fn3 | 984 . . . 1061 | 30/84 (36%) | 4.3e−16 |
| | | 65/84 (77%) | |
| fn3 | 1073 . . . 1156 | 26/87 (30%) | 2.8e−14 |
| | | 65/87 (75%) | |
| fn3 | 1164 . . . 1242 | 23/85 (27%) | 3.4e−13 |
| | | 58/85 (68%) | |
| fn3 | 1255 . . . 1334 | 26/85 (31%) | 3.4e−15 |
| | | 65/85 (76%) | |
| fn3 | 1346 . . . 1429 | 21/87 (24%) | 3.6e−13 |
| | | 64/87 (74%) | |
| fn3 | 1437 . . . 1513 | 20/85 (24%) | 8e−08 |
| | | 56/85 (66%) | |
| fn3 | 1528 . . . 1607 | 22/85 (26%) | 3.2e−11 |
| | | 58/85 (68%) | |
| fn3 | 1619 . . . 1698 | 21/85 (25%) | 2e−12 |
| | | 61/85 (72%) | |
| fn3 | 1709 . . . 1787 | 29/84 (35%) | 4.4e−17 |
| | | 58/84 (69%) | |
| fn3 | 1798 . . . 1875 | 23/84 (27%) | 8.5e−14 |
| | | 60/84 (71%) | |
| fn3 | 1886 . . . 1963 | 31/84 (37%) | 3.3e−19 |
| | | 60/84 (71%) | |
| fibrinogen_C | 1979 . . . 2187 | 121/272 (44%) | 2.1e−134 |
| | | 208/272 (76%) | |

### Example 36

The NOV36 clone was analyzed, and the nucleotide and encoded polypeptide sequences are shown in Table 36A.

TABLE 36A
NOV36 Sequence Analysis

| | |
|---|---|
| SEQ ID NO: 187 | 4077 bp |

| | |
|---|---|
| NOV36a, | GGAGCGGCGGGCGGGCGGGAGGGCTGGCGGGGCGAACGTCTGGGAGACGTCTGAAAGACC |
| CG56054-01 | |
| DNA Sequence | AACGAGACTTTGGACACCAGAGACGCGCCTGGCGGCACCTGGGGCTTGCGGCGTGCGAGA |
| | TTTCCCTTGCATTCGCTGGGAGCTCGCGCAGGGATCGTCCCATGGCCGGGGCTCGGAGCC |
| | GCGACCCTTGGGGCGCCTCCGGGATTTGCTACCTTTTTGGCTCCCTGCTCGTCGAACTGC |
| | TCTTCTCACGGCCTGTCCCCTTCAATCTGGACGTGATGGGTGCCTTGCGCAAGGAGGGCG |
| | AGCCAGGCAGCCTCTTCGGCTTCTCTGTGCCCCTGCACCCGCAGTTGCAGCCCCGACCCC |
| | AGAGCTCGCTGCTGCTGGGTGCTCCCCAGGCCCTGCCTCTTCCTGGGCAGCAGGCGAATC |
| | GCACTGGAGGCCTCTTCGCTTGCCCGTTGAGCCTGGAGGAGACTGACTGCTACAGACTGG |
| | ACATCGACCAGGCAGCTGATATGCAAAAGGAAAGCAAGGAGAACCAGTGGTTGCGAGTCA |
| | GTGTTCGGAGCCAGGCGCCTCCGGGCAAGATTGTTACCTGTGCACACCCATATCAGGCAA |
| | GGCAGCGAGTGGACCAGATCCTGGAGACGCGGCATATGATTGCTCGCTGCTTTGTGCTCA |
| | GCCAGGACCTGGCCATCCCGGATGAGTTGGATGGTGGGGAATGGAAGTTCTGTGAGGGAC |
| | GCCCCCAAGGCCATGAACAATTTCGGTTCTGCCAGCAGGGCACAGCTCCCCCCTTCTCCC |
| | CTGATACCCACTACCTCCTCTTTGGGCCCCCAGGAACCTATAATTGGAAGGGGTTGCTTT |
| | TTGTGACCAACATTCATAGCTCAGACCCCGACCAGCTGGTGTATAAAACTTTGCACCCTC |
| | CTGACCGGCTCCCAGGACCAGCCGCAGACTTGGCCCTCAATAGCTACTTAGGCTTCTCTA |
| | TTGACTCCGGCAAAGGTCTGGTGCGTGCAGAACAGCTGAGCTTTGTCGCTGGAGCCCCCC |
| | GCGCCAACCACAACGGTGCTGTGGTTATCCTGCGCAAGGACAGCGCCAGTCGCCTGGTGC |
| | CCGAGGTTATCCTGTCTGGCGAGCGCCTGACCTCCGGCTTTGGCTACTCACTGGCTGTGG |
| | CTGACCTCAACAGTCATCGCTGGCCACACCTGATAGTGGGTGCCCCCTACTTCTTTGAGC |
| | GCCAAGAAGAGCTGGGGGGTGCTGTGTATGTGTACTTCAACCAGGGGGGTCACTGGGCTG |
| | GGATCTCCCCTCTCCGGCTCTGCGGCTCCCCTCACTCCATGTTCGGGATCAGCCTGGCTG |
| | TCCTGGGGGACCTCAACCAAGATGGCTTTCCAGATATTGCAGTCGGTGCCCCCTTTGATG |
| | GTGATGGGAAAGTCTTCATCTACCATGGGAGCAGCCTGGGGGTTGTCGCCAAACCTTCAC |
| | AGGTGCTGGAGGGCGAGGCTGTGGGCATCAAGAGCTTCGGCTACTCCCTGTCAGGCAGCT |
| | TGGATATGGATGGGAACCAATACCCTGACCTGCTGGTGGGCTCCCTGGCTGACACCGCAG |
| | TGCTCTTCAGGGCCAGACCCATCCTCCATGTCTCCCATGAGGTCTCTATTGCTCCACGAA |
| | GCATCGACCTGGAGCAGCCCAACTGTGCTGGCGGCCACTCGGTCTGTGTGGACCTAAGGG |
| | TCTGTTTCAGCTACATTGCAGTCCCCAGCAGCTATAGCCCTACTGTGGCCCTGGACTATG |
| | TGTTAGATGCGGACACACACCGGAGGCTCCGGGGCCAGGTTCCCCGTGTGACGTTCCTGA |
| | GCCGTAACCTGGAAGAACCCAAGCACCAGGCCTCGGGCACCGTGTGGCTGAAGCACCAGC |
| | ATGACCGAGTCTCTGGAGACGCCATGTTCCAGCTCCAGGAAAATGTCAAAGACAAGCTTC |
| | GGGCCATTCTACTGACCTTGTCCTACACTCTCCAGACCCCTCGGCTCCGGCGACAGGCTC |
| | CTGGCCAGGGGCTCCCTCCAGTCGCCCCCATCCTCAATGCCCACCAGCCCAGCACCCAGC |
| | GGGCAGAGATCCACTTCCTCAAGCAAGGCTGTGGTGAAGACAAGATCTGCCAGAGCAATC |
| | TGCAGCTGGTCCACCCCCGCTTCTGTACCCGGGTCAGCGACACGGAATTCCAACCTCTGC |
| | CCATGGATGTGGATGGAACAACAGCCCTCTTTGCACTGAGTGGGCAGCCAGTCATTGGCC |
| | TGGAGCTGATGCTCACCAACCTGCCATCGGACCCAGCCCAGCCCCAGGCTGATGGGGATG |
| | ATGCCCATGAAGCCCAGCTCCTGGTCATGCTTCCTGACTCACTGCACTACTCAGGGGTCC |
| | GGGCCCTGGACCCTGCGGAGAACCCACTCTGCCTCTCCAATGAGAATGCCTCCCATGTTG |
| | AGTGTGAGCTGGGGAACCCCATGAAGAGAGGTGCCCAGGTCACCTTCTACCTCATCCTTA |
| | GCACCTCCGGGATCAGCATTGAGACCACGGAACTGGAGGTAGAGCTGCTGTTGGCCACGA |
| | TCAGTGAGCAGGAGCTGCATCCAGTCTCTGCACGAGCCCGTGTCTTCATTGAGCTGCCAC |
| | TGTCCATTGCAGGAATGGCCATTCCCCAGCAACTCTTCTTCTCTGGTGTGGTGAGCGGCG |
| | AGAGAGCCATGCACTCTGAGCGGGATGTCGGCAGCAAGGTCAAGTATGAGGTCACCGTTT |
| | CCAACCAAGCCCAGTCGCTCAGAACCCTGGGCTCTGCCTTCCTCAACATCATGTGGCCTC |
| | ATGAGATTGCCAATGGGAAGTGGTTGCTGTACCCAATGCAGGTTGAGCTGGAGGGCGGGC |
| | AGGGGCCTGGGCAGAAAGGGCTTTGCTCTCCCAGGCCCAACATCCTCCACCTGGATGTGG |
| | ACAGTAGGGATAGGAGGCGGCGCGAGCTGGAGCCACCTGAGCAGCAGGAGCCTGGTGAGC |
| | GGCAGGAGCCCAGCATGTCCTGGTGGCCAGTGTCCTCTGCTGAGAAGAAGAAAAACATCA |
| | CCCTGGACTGCGCCCGGGGCACGGCCAACTGTGTCCTGTTCAGCTGCCCACTCTACAGCT |
| | TTGACCGCGCGGCTGTGCTGCATGTCTGGGGCCGTCTCTGGAACAGCACCTTTCTGGAGG |
| | AGTACTCAGCTGTGAACTCCCTGGAAGTGATTGTCCGGGCCAACATCACAGTGAAGTCCT |
| | CCATAAAGAACTTGATGCTCCGAGATGCCTCCACAGTGATCCCAGTGATGGTATACTTGG |
| | ACCCCATGGCTCTCGTGGCAGAAGGAGTCCCCTGGTGGGTCATCCTCCTGGCTGTACTGG |
| | CTGGGCTGCTGGTGCTAGCACTGCTGGTGCTGCTCCTGTCGAAGATGGGATTCTTCAAAC |
| | GGGCGAAGCACCCCGAGGCCACCGTGCCCCAGTACCATGCGGTGAAGATTCCTCGGGAAG |
| | ACCGACAGCAGTTCAAGGAGGAGAAGACGCGCACCATCCTGAGGAACAACTGGGGCAGCC |
| | CCCGGCGGGAGGGCCCGGATGCACACCCCATCCTGGCTGCTGACGGGCATCCCGAGCTGG |
| | GCCCCGATGGGCATCCAGCGCCAGGCACCGCCTAGGTTCCCATGTCCCAGCCTGGCCTGT |
| | GGCTGCCCTCCATCCCTTCCCCAGAGATGGCTCCTTGGGATGAAGAGGGTAGAGTCCGCT |
| | GCTGGTGTCGCATCAAGATTTGGCACCATCCGCTTCCTCAGGGGCACAGACCTCTCCCAC |
| | CCACAAGAACTCCTCCCACCCAACTTCCCCTTAGAGTGCTGTGAGATGAGAGTGGGTAAA |
| | TCAGGGACAGGGCCATGGGGTAGGGTGAGAAGGGCAGGGGTGTCCTGATGCAAAGGTGGG |
| | GAGAAGGGATCCTAATCCCTTCCTCTCCCATTCACCCTGTGTAACAGGACCCCAAGGACC |
| | TGCCTCCCCGGAAGTGCCTTAACCTAGAGGGTCGGGGAGCAGGTTGTGTCACTGACTCAG |
| | GCTGCTCCTTCTCTAGTTTCCCCTCTCATCTGACCTTAGTTTGCTGCCATCAGTCTAGTG |
| | GTTTCGTGCTTTCGTCTATTTATTAAAAAATATTTGAGAACAAAAAAAAAAAAAAAA |

| | | |
|---|---|---|
| ORF Start: ATG at 162 | | ORF Stop: TAG at 3573 |
| SEQ ID NO: 188 | 1137 aa | MW at 124286.2 kD |

| | |
|---|---|
| NOV36a, | MAGARSRDPWGASGICYLFGSLLVELLFSRAVAFNLDVMGALRKEGEPCSLFGFSVALHR |
| CG56054.01 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCECRPQGHEQFGFCQQGTAAAFSPDSHYLLFGAPGTYNWKCLLFVTNIDSSDPDQLV |
| | YKTLDPADRLPGPAGDLALNSYLGFSIDSGKCLVRAEELSFVAGAPRANHKGAVVILRKD |
| | SASRLVPEVMLSCERLTSGFGYSLAVADLNSDGWPDLIVGAPYFFERQEELCGAVYVYLN |
| | QGGHWAGISPLRLCGSPDSMFGISLAVLGDLNQDGFPDIAVGAPFDGDGKVFIYHGSSLG |
| | VVAKPSQVLEGEAVGIKSFGYSLSGSLDMDGNQYPDLLVGSLADTAVLFRARPILHVSHE |
| | VSIAPRSIDLEQPNCAGGHSVCVDLRVCFSYIAVPSSYSPTVALDYVLDADTDRRLRCQV |
| | PRVTFLSRNLEEPKHQASGTVWLKHQHDRVCGDAMFQLQENVKDKLRAIVVTLSYSLQTP |
| | RLRRQAPGQGLPPVAPILNAHQPSTQRAEIHFLKQGCGEDKICQSNLQLVHARFCTRVSD |
| | TEFQPLPMDVDGTTALFALSGQPVIGLELMVTNLPSDPAQPQADGDDAHEAQLLVMLPDS |
| | LHYSGVRALDPAEKPLCLSNENASHVECELCNPMKRGAQVTFYLILSTSGISIETTELEV |
| | ELLLATTSEQELHPVSARARVFIELPLSTAGMAIPQQLFFSGVVRGERAMQSERDVGSKV |
| | KYEVTVSNQGQSLRTLGSAFLNIMWPHEIANGKWLLYPMQVELEGGQGPGQKGLCSPRPN |
| | ILHLDVDSRDRRRRELEPPEQQEPGERQEPSMSWWPVSSAEKKKNITLDCARGTANCVVF |
| | SCPLYSFDRAAVLHVWCRLWNSTFLEEYSAVKSLEVIVRANITVKSSIKNLMLRDASTVI |
| | PVMVYLDPMAVVAEGVPWWVILLAVLAGLLVLALLVLLLWKMGFFKRAKHPEATVPQYHA |
| | VKIPREDRQQFKEEKTGTILRNNWGSPRREGPDAHPILAADGHPELGPDCHPGPGTA |

| | |
|---|---|
| SEQ ID NO: 189 | 2564bp |

| | |
|---|---|
| NOV36b, | GGACCGCCGGGCGGGCGGGAGGGCTGGCGGGGCGAACGTCTCGGAGACGTCTGAAAGACC |
| CG56054-03 | |
| DNA Sequence | AACGAGACTTTGGAGACCAGAGACGCGCCTGGGGGGACCTGGGGCTTGGCGCGTGCGAGA |
| | TTTCCCTTGCATTCGCTGGGAGCTCGCGCAGGGATCGTCCCATGGCCGGGGCTCGGAGCC |
| | GCGACCCTTGGGGGGCCTCCGGGATTTGCTACCTTTTTGGCTCCCTGCTCGTCGAACTGC |
| | TCTTCTCACGGGCTGTCGCCTTCAATCTGGACGTGATGGGTCCCTTGCGCAAGGAGGGCG |
| | AGCCAGGCAGCCTCTTCGGCTTCTCTGTGGCCCTGCACCGGCAGTTGCAGCCCCGACCCC |
| | AGAGCTGGCTGCTGGTGGGTGCTCCCCAGGCCCTGGCTCTTCCTGGGCAGCAGGCGAATC |
| | GCACTGGAGGCCTCTTCGCTTGCCCGTTGAGCCTGGAGGAGACTGACTGCTACAGAGTGG |
| | ACATCGACCAGGGACCTGATATGCAAAAGGAAAGCAAGGAGAACCAGTGGTTGGGAGTCA |
| | GTGTTCGGAGCCAGGGGCCTGGGGGCAAGATTGTTACCTGTGCACACCGATATGAGGCAA |
| | GGCAGCGAGTCGACCAGATCCTGCAGACGCGGGATATGATTGGTCGCTGCTTTGTGCTCA |
| | GCCAGGACCTGGCCATCCGGGATGAGTTGGATGGTGGGGAATGGAAGTTCTGTGAGGGAC |
| | GCCCCCAAGGCCATGAACAATTTGGGTTCTGCCAGCAGGGCACAGCTGCCGCCTTCTCCC |
| | CTGATAGCCACTACCTCCTCTTTGGGGCCCCAGGAACCTATAATTGGAAGGGGTTGCTTT |
| | TTGTGACCAACATTGATAGCTCAGACCCCGACCAGCTGGTGTATAAAACTTTGGACCCTG |
| | CTGACCGGCTCCCAGGACCAGCCGGAGACTTGGCCCTCAATAGCTACTTAGGCTTCTCTA |
| | TTGACTCGGGGAAAGGTCTGGTGCGTGCAGAAGAGCTGAGCTTTGTGGCTGGAGCCCCCC |
| | GCGCCAACCACAAGGGTGCTGTGGTTATCCTGCGCAAGGACAGCGCCAGTCGCCTGGTGC |
| | CCGAGGTTATGCTCTCTGGGGAGCGCCTGACCTCCGGCTTTGGCTACTCACTGGCTGTGG |
| | CTGACCTCAACAGTGATGGCTGGCCAGACCTGATAGTGGGTGCCCCCTACTTCTTTGAGC |
| | GCCAAGAAGAGCTGGGGGGTGCTGTGTATGTGTACTTGAACCAGGGGGGTCACTGGGCTG |
| | GGATCTCCCCTCTCCGGCTCTGCAACTCCCCGCACTCCATGTTCGGGATCAGCCTGGCTG |
| | TCCTGGGGGACCTCAACCAAGATGGCTTTCCAGATATTGCAGTGGGTGCCCCCTTTGATG |
| | GTGATGGGAAAGTCTTCATCTACCATGGGAGCAGCCTGGGGGTTGTCGCCAAACCTTCAC |
| | AGGTGCTGGAGGGCGAGGCTGTGGGCATCAAGAGCTTCGGCTACTCCCTGTCAGGCAGCT |
| | TGGATATGGATGGGAACCAATACCCTGACCTGCTGCTGCGCTCCCTGGCTGACACCGCAG |
| | TGCTCTTCAGGGCCAGACCCATCCTCCATGTCTCCCATGAGGTCTCTATTGCTCCACGAA |
| | GCATCGACCTGGAGCAGCCCAACTGTGCTGGCCGCCACTCGGTCTGTGTGGACCTAAGGG |
| | TCTGTTTCAGCTACATTGCAGTCCCCAGCAGCTATAGCCCTACTGTGGCCCTGGACTATG |
| | TGTTAGATGCGGACACAGACCCGAGGCTCCGGGGCCAGGTTCCCCGTGTGACCTTCCTGA |
| | GCCGTAACCTGGAAGAACCCAAGCACCAGGCCTCGGGCACCGTGTGGCTGAAGCACCAGC |
| | ATGACCGACTCTGTGGAGACGCCATGTTCCAGCTCCAGGAAAATGTCAAAGACAAGCTTC |
| | GGGCCATTCTACTGACCTTGTCCTACAGTCTCCAGACCCCTCGGCTCCGGCGGGAGGGCC |
| | CGGATGCACACCCCATCCTGGCTGCTCACGGGCATCCCGAGCTGGCCCCCGATGGCCATC |
| | CAGGGCCACGCACCGCCTAGGTTCCCATGTCCCAGCCTGGCCTGTGGCTGCCCTCCATCC |
| | CTTCCCCAGAGATGGCTCCTTGGGATGAAGAGGGTAGAGTGGGCTGCTGGTGTCGCATCA |
| | AGATTTGCCAGGATCGGCTTCCTCAGGGGCACAGACCTCTCCCACCCACAAGAACTCCTC |
| | CCACCCAACTTCCCCTTAGAGTGCTGTGAGATGAGAGTGGGTAAATCAGGGACAGGGCCA |
| | TGGGGTAGGGTGAGAAGGGCAGGGGTGTCCTGATGCAAAGGTGGGGAGAAGGCATCCTAA |
| | TCCCTTCCTCTCCCATTCACCCTGTGTAACAGGACCCCAAGGACCTGCCTCCCCGGAAGT |
| | GCCTTAACCTAGAGGGTCGGGGACGAGGTTGTGTCACTGACTCAGGCTGCTCCTTCTCTA |
| | GTTTCCCCTCTCATCTGACCTTAGTTTCCTGCCATCAGTCTAGTGGTTTCGTGGTTTCGT |
| | CTATTTATTAAAAAATATTTGAGAACAAAAAAAAAAAAAAAAAA |

| | | |
|---|---|---|
| ORF Start: ATG at 162 | | ORF Stop: TAG at 2058 |
| SEQ ID NO: 190 | 632 aa | MW at 68332.4 kD |

| | |
|---|---|
| NOV36b, | MAGARSRDPWGASGICYLFGSLLVELLFSRAVAFNLDVMGALRKEGEPGSLFGFSVALHR |
| CG56054-03 | |
| Protein Sequence | QLQPRPQSWLLVGAPQALALPGQQANRTGGLFACPLSLEETDCYRVDIDQCADMQKESKE |
| | NQWLCVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGLLFVTNIDSSDPDQLV |
| | YKTLDPADRLPGPAGDLALNSYLGFSTDSGKGLVRAEELSFVAGAPRANHKGAVVILRKD |
| | SASRLVPEVMLSGERLTSGFGYSLAVADLNSDGWPDLIVGAPYFFERQEELGGAVYVYLN |
| | QGGHWAGISPLRLCNSPHSMFGISLAVLGDLNQDGFPDIAVGAPFDGDCKVFIYHGSSLG |
| | VVAKPSQVLEGEAVGIKSPGYSLSCSLDMDGNQYPDLLVGSLADTAVLFRARPILHVSHE |
| | VSIAPRSIDLEQPNCAGGHSVCVDLRVCFSYIAVPSSYSPTVALDYVLDADTDRRLRGQV |
| | PRVTFLSRNLEEPKHQASGTVWLKHQHDRVCGDAMFQLQENVKDKLRAIVVTLSYSLQTP |
| | RLRREGPDAHPILAADGHPELGPDGHPGPGTA |

| | |
|---|---|
| SEQ ID NO: 191 | 2017 bp |

| | |
|---|---|
| NOV36c, | GGAGCGGCGGGCGGGCGGGAGGGCTGGCGGGGCGAACGTCTGGGAGACGTCTGAAAGACC |
| CG56054-04 | |
| DNA Sequence | AACGAGACTTTGGAGACCAGAGACGCGCCTGGGGGGACCTGGGGCTTGGGGCGTGCGAGA |
| | TTTCCCTTGCATTCGCTGGGAGCTCGCGCAGGGATCGTCAAAATGGCCGGGGCTCGGAGCC |
| | GCGACCCTTGGGGGGCCTCCGGGATTTGCTACCTTTTTGGCTCCCTGCTCGTCGAACTGC |
| | TCTTCTCACGGGCTGTCGCCTTCAATCTGGACGTGATGGGTGCCTTGCGCAAGGAGGGCG |
| | AGCCAGGCAGCCTCTTCGGCTTCTCTGTGGCCCTGCACCGGCAGTTGCAGCCCCGACCCC |
| | AGAGCTGGCTGCTGGTGGGTGCTCCCCAGGCCCTGGCTCTTCCTGGGCAGCAGGCGAATC |
| | GCACTGGAGGCCTCTTCGCTTGCCCGTTGAGCCTGGAGGAGACTGACTGCTACAGAGTCG |
| | ACATCGACCAGGGAGCTGATATGCAAAAGGAAAGCAAGGAGAACCAGTGGTTGGGAGTCA |
| | GTGTTCGGAGCCAGGGGCCTGGGGGCAAGATTGTTACCTGTCCACACCGATATGAGGCAA |
| | GGCAGCGAGTGGACCAGATCCTGGAGACCCGGGATATGATTGGTCGCTGCTTTGTGCTCA |
| | GCCAGGACCTGGCCATCCGGGATGAGTTGGATGGTGGGGAATGGAAGTTCTGTGAGGGAC |
| | GCCCCCAAGGCCATGAACAATTTGGGTTCTGCCAGCAGGGCACAGCTGCCGCCTTCTCCC |
| | CTGATAGCCACTACCTCCTCTTTGGGGCCCCAGGAACCTATAATTGGAAGGGGTTGCTTT |
| | TTGTGACCAACATTCATAGCTCAGACCCCGACCACCTGGTGTATAAAACTTTGGACCCTG |
| | CTGACCGGCTCCCAGGACCAGCCGGAGACTTGCCCCTCAATAGCTACTTAGGCTTCTCTA |
| | TTGACTCGGGGAAAGGTCTGGTGCCTGCAGAAGAGCTGAGCTTTGTGGCTGGAGCCCCCC |
| | GCGCCAACCACAAGGGTGCTQTGPTCATCCTGCGCAAGGAPAGCGCCAGTCGCCTGCTGC |
| | CCGAGCTTATGCTGTCTGGCGAGCGCCTGACCTCCGGCTTTCGCTACTCACTCCCTGTGG |
| | CTGACCTCAACAGTGATCCCTGGCCAGACCTGATAGTGGGTGCCCCCTACTTCTTTGAGC |
| | GCCAAGAAGAGCTGGGGGGTGCTGTGTATGTGTACTTGAACCAGGGGGGTCACTGGGCTG |
| | GGATCTCCCCTCTCCGGCTCTGCGGCTCCCCTGACTCCATGTTCGGGATCAGCCTGGCTG |
| | TCCTGGGGGACCTCAACCAAGATGGCCTTCCAGATATTGCAGTGGGTGCCCCCTTTGATG |
| | GTGATGGGAAAGTCTTCATCTACCATGGGAGCAGCCTGGGGGTTGTCGCCAAGCCTTCAC |
| | AGGTGCTGGAGGGCGAGGCTGTCGGCATCCCGACCTGGGCCCCGATGCGCATCCAGCCCC |
| | AGGCACCGCCTACCTTCCCATGTCCCACCCTGGCCTGTGGCTGCCCTCCATCCCTTCCCC |
| | AGAGATGGCTCCTTCGGATGAAGAGGGTAGAGTGCGCTGCTGGTGTCGCATCAAGATTTG |
| | GCAGGATCGGCTTCCTCAGGGGCACAGACCTCTCCCACCCACAAGAACTCCTCCCACCCA |
| | ACTTCCCCTTAGAGTGCTGTGAGATGAGAGTGGGTAAATCAGGGACAGGGCCATGGGGTA |
| | GGGTGAGAAGGGCAGGGGTGTCCTGATGCAAAGGTGGGGAGAAGGGATCCTAATCCCTTC |
| | CTCTCCCATTCACCCTGTGTAACAGGACCCCAAGGACCTGCCTCCCCGGAAGTGCCTTAA |
| | CCTAGAGGGTCGGGGAGGAGGTTGTGTCACTGACTCAGGCTGCTCCTTCTCTAGTTTCCC |
| | CTCTCATCTGACCTTAGTTTGCTGCCATCAGTCTAGTGGTTTCGTGGTTTCGTCTATTTA |
| | TTAAAAAATATTTCACAAAAAAAAAAAAAAAAAAAAA |

| | | |
|---|---|---|
| ORF Start: ATG at 162 | | ORF Stop: TGA at 1764 |
| SEQ ID NO: 192 | 534 aa | MW at 57440.7 kD |

| | |
|---|---|
| NOV36c, | MAGARSRDPWGASCICYLFGSLLVELLFSRAVAFNLDVMGALRKEGEPGSLFGFSVALHR |
| CG56054-04 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGLLFVTNIDSSDPDQLV |
| | YKTLDPADRLPGPAGDLALNSYLGFSIDSGKGLVRAEELSFVAGAPRANHKGAVVILRKD |
| | SASRLVPEVMLSGERLTSCFGYSLAVADLNSDGWPDLIVGAPYFFERQEELGGAVYVYLN |
| | QGGHWAGTSPLRLCGSPDSMFGISLAVLGDLNQDGLPDIAVGAPFDGDGKVPTYHGSSLG |
| | VVAKPSQVLEGEAVGIPSWAPMGIQGQAPPRFPCPSLACGCPPSLPQRWLLGMKRVEWAA |
| | GVASRFGRIGFLRGTDLSHFQELLPPNFPLECCEMRVGKSGTGPWGRVRRAGVS |

| | |
|---|---|
| SEQ ID NO: 193 | 999 bp |

| | |
|---|---|
| NOV36d, | ATGGCCGGGGCTCGGAGCCGCCACCCTTGGGGGGCCTCCGGGATTTGCTACCTTTTTGGC |
| CG56054-05 | |
| DNA Sequence | TCCCTGCTCGTCCAACTGCTCTTCTCACGGGCTGTCGCCTTCAATCTGGACGTGATGGGT |
| | GCCTTGCGCAAGGAGGGCGAGCCAGGCAGCCTCTTCGGCTTCTCTCTGGCCCTGCACCGG |
| | CAGTTGCAGCCCCGACCCCAGAGCTGGCTGCTGGTGGGTGCTCCCCAGGCCCTCGCTCTT |
| | CCTGGGCAGCAGCCGAATCGCACTGGAGGCCTCTTCGCTTGCCCGTTGAGCCTGGAGGAG |
| | ACTGACTGCTACAGAGTGGACATCGACCAGGGAGCTGATATGCAAAAGGAAAGCAAGGAG |
| | AACCAGTCGTTGGGAGTCACTGTTCGGAGCCAGCGGCCTGGGGGCAAGATTGTTACCTGT |
| | GCACACCCCATCCTGGCTGCTGACGCGCATCCCGAGCTGCGCCCCGATGGCCATCCAGGG |
| | CCACGCACCGCCTAGGTTCCCATGTCCCAGCCTGCCCTGTGGCTGCCCTCCATCCCTTCC |
| | CCAGAGATGGCTCCTTGGGATGAAGAGGGTAGAGTGGGCTGCTGGTGTCGCATCAAGATT |
| | TGGCAGGATCGGCTTCCTCAGGGGCACAGACCTCTCCCACCCACAAGAACTCCTCCCACC |
| | CAACTTCCCCTTAGAGTGCTGTCACATGAGAGTGCGTAAATCAGGCACAGGGCCATGGGG |
| | TAGGGTGAGAAGGGCAGGGGTGTCCTGATGCAAAGGTGGGGAGAAGGGATCCTAATCCCT |
| | TCCTCTCCCATTCACCCTGTCTAACAGGACCCCAAGGACCTGCCTCCCCGGAAGTGCCTT |
| | AACCTACAGGGTCGGGGACGAGGTTGTGTCACTGACTCACGCTGCTCCTTCTCTAGTTTC |
| | CCCTCTCATCTGACCTTAGTTTGCTGCCATCAGTCTAGTGGTTTCGTGGTTTCGTCTATT |
| | TATTAAAAATATTTGAGAACAAAAAAAAAAAAAAAAAAA |

| | | |
|---|---|---|
| ORF Start: ATG at 1 | | ORF Stop: TAG at 493 |
| SEQ ID NO: 194 | 164 aa | MW at 17332.5 kD |

| | |
|---|---|
| NOV36d, | MAGARSRDPWGASGICYLFGSLLVELLFSRAVAFNLDVMGALRKEGEPGSLFGF |
| CG56054-05 | |
| Protein Sequence | SVAL |
| | HRQLQPRPQSWLLVGAPQALALPGQQANRTGGLFACPLSLEETDCYRVDIDQGA |
| | DMQK |
| | ESKENQWLGVSVSQGPGCKTVTCAHPILAADGHPELGPDGHPGPGTA |

| | |
|---|---|
| SEQ ID NO: 195 | 2701 bp |

| | |
|---|---|
| NOV36e, | GGAGCGGCGGGCGCGCGGGAGGGCTGGCGGGGCGAACGTCTGGGAGACGTCTGAAAGACC |
| CG56054-06 | |
| DNA Sequence | AACGAGACTTTGGAGACCAGAGACGCGCCTGGGCGGACCTGGGGCTTGGGGCGTGCGAGA |
| | TTTCCCTTCCATTCGCTGGGACCTCGCGCAGGGATCGTCCCATGGCCGGGGCTCGGAGCC |
| | GCGACCCTTGCGGGGCCTCCGGGATTTGCTACCTTTTTGGCTCCCTGCTCGTCGAACTGC |
| | TCTTCTCACGGGCTGTCGCCTTCAATCTGGACGTGATGGCTGCCTTGCGCAAGGAGGGCG |
| | AGCCAGGCAGCCTCTTCGGCTTCTCTGTGGCCCTGCACCGGCAGTTGCAGCCCTGGACTA |
| | TGTGTTAGATGCGGACACAGACCGGACGCTCCGGGGCCAGGTTCCCCGTGTGACGTTCCT |
| | GAGCCGTAACCTGCAAGAACCCAAGCACCAGGCCTCGGGCACCGTGTGGCTGAAGCACCA |
| | GCATGACCGAGTCTCTGGAGACGCCATGTTCCAGCTCCAGGAAAATGTCAAAGACAAGCT |
| | TCGGGCCATTCTAGTGACCTTGTCCTACAGTCTCCAGACCCCTCGGCTCCGGCGACAGGC |
| | TCCTGGCCAGGGGCTGCCTCCAGTGGCCCCCATCCTCAATGCCCACCAGCCCAGCACCCA |
| | GCGGGCACAGATCCACTTCCTGAACCAAGGCTGTCGTGAAGACAAGATCTGCCACACCAA |
| | TCTGCAGCTGCTCCACGCCCGCTTCTGTACCCGGGTCACCGACACGCAATTCCAACCTCT |
| | GCCCATGGATGTGGATGCAACAACAGCCCTGTTTGCACTGAGTGGGCAGCCAGTCATTGG |
| | CCTGGAGCTGATGGTCACCAACCTGCCATCGGACCCAGCCCAGCCCCAGGCTGATGGGGA |
| | TGATGCCCATGAAGCCCAGCTCCTGGTCATGCTTCCTGACTCACTGCACTACTCAGGGGT |
| | CCGGGCCCTGGACCCTCCGGAGAAGCCACTCTGCCTGTCCAATGAGAATGCCTCCCATGT |
| | TGAGTGTCAGCTGGCGAACCCCATGAACAGAGGTCCCCAGCTCACCTTCTACCTCATCCT |
| | TAGCACCTCCGGCATCAGCATTGAGACCACGGAACTGGAGGTAGAGCTGCTGTTGGCCAC |
| | GATCAGTGAGCAGGAGCTGCATCCAGTCTCTGCACGAGCCCGTGTCTTCATTGACCTGCC |
| | ACTGTCCATTGCAGGAATGCCCATTCCCCAGCAACTCTTCTTCTCTGGTGTGGTGAGGGG |
| | CGAGAGAGCCATCCAGTCTGAGCGGGATCTGGGCAGCAAGGTCAAGTATGAGGTCACGGT |
| | TTCCAACCAAGGCCAGTCGCTCAGAACCCTGGCCTCTGCCTTCCTCAACATCATGTGGCC |
| | TCATCAGATTGCCAATGGGAACTGGTTGCTGTACCCAATGCAGGTTGAGCTGGAGGGCGG |
| | GCACGCGCCTGGGCAGAAAGGGCTTTCCTCTCCCAGGCCCAACATCCTCCACCTGGATGT |
| | GGACAGTACGGATAGGAGGCGGCGCGAGCTGGAGCCACCTGAGCAGCAGGAGCCTCGTGA |
| | GCGGCAGGAGCCCAGCATGTCCTGGTGGCCAGTGTCCTCTGCTGAGAAGAAGAAAAACAT |
| | CACCCTGGACTGCGCCCCGGGCACCGCCAACTGTGTGGTGTTCACCTGCCCACTCTACAG |
| | CTTTGACCGCCCGGCTGTGCTGCATGTCTGGGGCCGTCTCTGGAACAGCACCTTTCTGGA |
| | GGAGTACTCAGCTCTGAAGTCCCTGGAAGTGATTGTCCCGCCCAACATCACAGTGAAGTC |
| | CTCCATAAAGAACTTGATGCTCCGACATGCCTCCACAGTGATCCCAGTCATGGTATACTT |
| | GGACCCCATGGCTGTGGTGGCAGAAGCAGTGCCCTGGTGGGTCATCCTCCTGGCTGTACT |
| | GGCTGGGCTGCTGGTGCTAGCACTGCTGGTGCTGCTCCTGTGGAAGATGGGATTCTTCAA |
| | ACGGGCGAAGCACCCCGAGGCCACCGTGCCCCAGTACCATGCGGTGAACATTCCTCGGGA |
| | AGACCGACAGCAGTTCAAGGAGGAGAAGACGGGCACCATCCTCAGGAACAACTGGGGCAG |
| | CCCCCGGCGGGAGGCCCCGGATGCACACCCCATCCTGGCTGCTGACGGGCATCCCGAGCT |
| | GGGCCCCGATCGGCATCCAGGGCCAGGCACCGCCTAGGTTCCCATGTCCCAGCCTGGCCT |
| | GTGGCTCCCCTCCATCCCTTCCCCAGAGATGGCTCCTTGCGATGAAGACGGTAGAGTCGG |
| | CTGCTCGTGTCCCATCAAGATTTGGCAGGATCGGCTTCCTCAGGCGCACACACCTCTCCC |
| | ACCCACAAGAACTCCTCCCACCCAACTTCCCCTTAGAGTGCTGTGAGATGAGACTGCGTA |
| | AATCAGGGACAGCGCCATGCGGTAGGGTGAGAAGGGCAGCGGTGTCCTGATGCAAAGGTG |
| | GGGAGAAGGGATCCTAATCCCTTCCTCTCCCATTCACCCTGTGTAACAGCACCCCAAGGA |
| | CCTGCCTCCCCGGAAGTGCCTTAACCTACAGGGTCGCGGACCACGTTGTGTCACTGACTC |
| | AGGCTGCTCCTTCTCTAGTTTCCCCTCTCATCTGACCTTAGTTTGCTGCCATCAGTCTAG |
| | TGGTTTCGTGCTTTCGTCTATTTATTAAAAAATATTTGAGAACAAAAAAAAAAAAAAAAA |
| | A |

| | | |
|---|---|---|
| ORF Start: ATG at 162 | | ORF Stop: TAG at 366 |
| SEQ ID NO: 196 | 68 aa | MW at 7433.6 kD |

| | |
|---|---|
| NOV36e, | MAGARSRDPWGASGICYLFGSLLVELLFSRAVAFNLDVMGALRKEGEPGSLFGFSVALHR |
| CG56054-06 | |
| Protein Sequence | QLQPWTMC |

| | |
|---|---|
| SEQ ID NO: 197 | 1131 bp |

| | |
|---|---|
| NOV36f, | GGAGCGGCGGGCGGGCGCGAGGGCTGGCGGGGCGAACGTCTGGGAGACGTCTGAAAGACC |
| CG56054-07 | |
| DNA Sequence | AACGAGACTTTGGAGACCAGAGACGCGCCTGGGGGGACCTGGGGCTTGGGGCGTGCGAGA |
| TTTCCCTTGCATTCGCTGGGAGCTCGCGCAGGGATCCTCCCATGGCCGGGGCTCGGAGCC | |
| | GCGACCCTTGGGGGGCCTCCGGGATTTGCTACCTTTTTGGCTCCCTCCTCGTCGAACTGC |
| | TCTTCTCACCGGCTGTCGCCTTCAATCTGGACGTGATGGGTGCCTTGCGCAAGGAGGGCG |
| | AGCCAGGCACCCTCTTCGGCTTCTCTGTGGCCCTGCACCGGCAGTTGCAGCCCCGACCCC |
| | AGAGCTGGCTGCTGGTGGGTGCTCCCCAGGCCCTGGCTCTTCCTGGGCAGCAGGCGAATC |
| | GCACTGGAGGCCTCCGTGCCCCAGTACCATGCGGTGAAGATTCCTCGGGAAGACCGACAG |
| | CAGTTCAAGGAGGAQAAGACGGGCACCATCCTGAGGAACAACTGGGGCAGCCCCCGCCGG |
| | GAGGGCCCGGATGCACACCCCATCCTGGCTGCTGACGCGCATCCCCAGCTGGGCCCCGAT |
| | GGCCATCCAGGGCCAGGCACCGCCTAGGTTCCCATGTCCCAGCCTGGCCTCTGGCTGCCC |
| | TCCATCCCTTCCCCACACATGGCTCCTTCGGATGAAGACGGTAGAGTGGGCTGCTGGTGT |
| | CGCATCAAGATTTGCCAGGATCGGCTTCCTCAGGGGCACAGACCTCTCCCACCCACAAGA |
| | ACTCCTCCCACCCAACTTCCCCTTAGAGTGCTGTGAGATGAGAGTGGGTAAATCAGGGAC |
| | AGGGCCATCGGGTAGGGTGAGAAGGGCAGGGGTGTCCTGATGCAAAGGTGGGGAGAAGCG |
| | ATCCTAATCCCTTCCTCTCCCATTCACCCTGTCTAACAGGACCCCAAGGACCTGCCTCCC |
| | CGGAAGTGCCTTAACCTAGAGCGTCGGCGAGGAGGTTGTGTCACTGACTCAGGCTGCTCC |
| | TTCTCTAGTTTCCCCTCTCATCTGACCTTAGTTTGCTGCCATCAGTCTAGTGGTTTCGTG |
| | GTTTCGTCTATTTATTAAAAATATTTGAGAACAAAAAAAAAAAAAAAAAAA |

| | | |
|---|---|---|
| ORF Start: ATG at 162 | | ORF Stop: TGA at 573 |
| SEQ ID NO: 198 | 137 aa | MW at 14203.9 kD |

| | |
|---|---|
| NOV36f, | MAGARSRDPWGASCICYLFGSLLVELLFSRAVAFNLDVMGALRKECEPGSLFGFSVALHR |
| CG56054-07 | |
| Protein Sequence | QLQPRPQSWLLVGAPQALALPGQQANRTGGLRAPVPCGEDSSGRPTAVQGGEDGHHPEEQ |
| | LGQPPAGGPGCTPHPGC |

| | |
|---|---|
| SEQ ID NO: 199 | 2175 bp |

| | |
|---|---|
| NOV36g, | GGAGCGGCGGGCGGGCGGGAGGGCTGGCGGGGCGAACGTCTGGGAGACGTCTGAAAGACC |
| CG56054-08 | |
| DNA Sequence | AACGAGACTTTGGAGACCAGAGACGCGCCTGGGGGGACCTGGGGCTTGGCCCGTGCGAGA |
| | TTTCCCTTGCATTCGCTGGCAGCTCCCGCAGCGATCGTCCCATGGCCGGGGCTCGGAGCC |
| | GCGACCCTTGGGGCGCCTCCGGGATTTCCTACCTTTTTGGCTCCCTGCTCGTCGAACTCC |
| | TCTTCTCACGGGCTGTCGCCTTCAATCTGGACGTGATCGGTGCCTTCCGCAAGGACGGCG |
| | AGCCAGGCAGCCTCTTCGGCTTCTCTGTGGCCCTGCACCGGCAGTTGCAGCCCCCACCCC |
| | AGAGCTGGCTGCTGCTGGGTGCTCCCCAGGCCCTGGCTCTTCCTGGGCAGCAGGCGAATC |
| | GCACTGGAGGCCTCTTCGCTTGCCCGTTGAGCCTGGAGGAGACTGACTGCTACAGAGTGG |
| | ACATCGACCAGGGAGCTCATATGCAAAAGGAAAGCAAGGAGAACCAGTGGTTCGGAGTCA |
| | GTGTTCGGAGCCAGGGGCCTGGGGGCAAGATTGTTACCTGTCCACACCGATATGACCCAA |
| | GGCAGCGAGTGGACCAGATCCTGGAGACGCGGGATATGATTGCTCGCTGCTTTGTGCTCA |
| | GCCACGACCTGGCCATCCGCCATGAGTTGGATGGTGGGGAATGGAAGTTCTGTGAGGGAC |
| | GCCCCCAAGGCCATGAACAATTTGGGTTCTGCCAGCAGGGCACAGCTGCCGCCTTCTCCC |
| | CTGATAGCCACTACCTCCTCTTTGGGGCCCCAGGAACCTATAATTGGAAGGGGTTGCTTT |
| | TTGTGACCAACATTGATAGCTCAGACCCCGACCAGCTGGTGTATAAAACTTTCGACCCTG |
| | CTGACCGGCTCCCAGGACCAGCCGGAGACTTCGCCCTCAATAGCTACTTACGCTTCTCTA |
| | TTGACTCGGGGAAAGGTCTGGTCCGTGCAGAAGACCTCAGCTTTGTGGCTGGAGCCCCCC |
| | GCGCCAACCACAAGGGTGCTGTGGTCATCCTCCGCAAGGACACCGCCAGTCGCCTGGTGC |
| | CCGAGGTTATGCTGTCTGGGGAGCGCCTGACCTCCGGCTTTGGCTACTCACTCCCTGTGG |
| | CTGACCTCAACAGTCATGGCTGGCCAGACCTGATAGTGGGTGCCCCCTACTTCTTTGAGC |
| | GCCAAGAAGAGCTGGGGGGTGCTGTGTATGTGTACTTGAACCAGGGGGGTCACTGGGCTG |
| | GGATCTCCCCTCTCCGGCTCTGCCGCTCCCCTGACTCCATGTTCGGGATCAGCCTGCCTG |
| | TCCTGGGCGACCTCAACCAAGATGGCTGTGGTGCCAGAAGGAGTGCCCTGGTGGGTCATC |
| | CTCCTGGCTGTACTGGCTCGGCTGCTGGTGCTACCACTGCTCGTGCTGCTCCTGTGGAAG |
| | ATGGGATTCTTCAAACGGGCCAAGCACCCCGAGGCCACCGTGCCCCAGTACCATGCGGTG |
| | AAGATTCCTCGGGAAGACCGACAGCAGTTCAAGGAGGAGAAGACGGGCACCATCCTGAGG |
| | AACAACTGGGGCAGCCCCCGGCGGGAGGGCCCGGATGCACACCCCATCCTGGCTGCTGAC |
| | GGGCATCCCGAGCTGGGCCCCGATGGGCATCCAGGGCCAGGCACCGCCTAGCTTCCCATG |
| | TCCCAGCCTGGCCTGTGGCTGCCCTCCATCCCTTCCCCAGAGATGGCTCCTTGGGATGAA |
| | GAGGGTAGAGTGGGCTGCTGGTGTCGCATCAAGATTTGGCAGGATCGGCTTCCTCAGGGG |
| | CACAGACCTCTCCCACCCACAAGAACTCCTCCCACCCAACTTCCCCTTAGAGTGCTGTGA |
| | GATGAGAGTGGGTAAATCAGGGACAGGGCCATGGCGTAGGGTGAGAAGGGCAGGGGTGTC |
| | CTGATGCAAAGGTGGGGAGAAGGGATCCTAATCCCTTCCTCTCCCATTCACCCTGTGTAA |
| | CAGGACCCCAAGGACCTGCCTCCCCGGAAGTGCCTTAACCTAGAGGGTCGGGGAGGAGGT |
| | TGTGTCACTGACTCAGGCTGCTCCTTCTCTAGTTTCCCCTCTCATCTGACCTTAGTTTGC |
| | TGCCATCAGTCTAGTGGTTTCGTGGTTTCGTCTATTTATTAAAAAATATTTGAGAACAAA |
| | AAAAAAAAAAAAAAA |

| | | |
|---|---|---|
| ORF Start: ATG at 162 | | ORF Stop: TGA at 1617 |
| SEQ ID NO: 200 | 485 aa | MW at 51430.2 kD |

| | |
|---|---|
| NOV36g, | MAGARSRDPWGASGICYLFGSLLVELLFSRAVAFNLDVMGALRKEGEPGSLFGFSVALHR |
| CG56054-08 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGLLFVTNIDSSDPDQLV |
| | YKTLDPADRLPGPAGDLALNSYLGFSIDSGKGLVRAEELSFVAGAPRANHKGAVVILRKD |
| | SASRLVPEVMLSGERLTSGFGYSLAVADLNSDGWPDLIVGAPYFFERQEELGCAVYVYLN |
| | QGGHWAGISPLRLCGSPDSMFGISLAVLGDLNQDGCGGRRSALVGHPPGCTGWAAGASTA |
| | GAAPVEDGILQTGEAPRGHRAPVPCGEDSSGRPTAVQGGEDGHHPEEQLGQPPAGGPGCT |
| | PHPGC |

| | |
|---|---|
| SEQ ID NO: 201 | 1458 bp |

| | |
|---|---|
| NOV36h, | TTCGGGCGTGCGAGATTTCCCTTGCATTCGCTGGGAGCTCGCGCACGCATCGTCCCATGG |
| CG56054-09 | |
| DNA Sequence | CCGGGGCTCGGAGCCGCGACCCTTCGGGGGCCTCCGCGATTTGCTACCTTTTTCGCTCCC |
| | TGCTCGTCGAACTGCTCTTCTCACGGGCTGTCGCCTTCAATCTGCACGTGATGGGTGCCT |
| | TGCGCAAGGACGGCGAGCCAGCCAGCCTCTTCGGCTTCTCTGTGGCCCTGCACCGGCAGT |
| | TGCACCCCCGACCCCAGAGCTCGCTGCTGGTGGGTGCTCCCCAGCCCCTGCCTCTTCCTG |
| | GGCAGCACCCGAATCGCACTGGAGGCCTCTTCCCTTGCCCGTTGAGCCTGGAGGAGACTG |
| | ACTGCTACAGAGTGGACATCGACCAGGGAGCTGATATGCAAAAGGAAAGCAAGGAGAACC |
| | AGTGGTTGGGAGTCAGTGTTCCGAGCCAGGGGCCTGGGGGCAAGATTGTTACCTGTGCAC |
| | ACCGATATGAGGCAAGCCAGCGAGTCGACCAGATCCTGGACACGCGGGATATCATTCGTC |
| | GCTGCTTTGTGCTCACCCAGCACCTGGCCATCCGGGATGAGTTGGATGGTCCGGAATGGA |
| | AGTTCTGTGAGGGACGCCCCCAAGGCCATGAACAATTTGGGTTCTGCCAGCACGGCACAG |
| | CTGCCGCCTTCTCCCCTCATAGCCACTACCTCCTCTTTGGGGCCCCAGCAACCTATAATT |
| | GGAAGGGCACGGCCAGGGTGGAGCTCTGTGCACAGGGCTCAGCGGACCTGGCACACCTGG |
| | ACGACGGTCCCTACGAGGCGGGGGGAGAGAAGGAGCAGGACCCCCGCCTCATCCCGGTCC |
| | CTGCGAACAGCACCTTTCTGGAGGAGTACTCAGCTGTGAAGTCCCTGGAAGTGATTGTCC |
| | GGGCCAACATCACAGTGAAGTCCTCCATAAAGAACTTGATCCTCCGAGATGCCTCCACAG |
| | TGATCCCAGTGATGGTATACTTCGACCCCATGGCTGTGGTGGCAGAAAGAGTGCCCTGGT |
| | GGGTCATCCTCCTCGCTGTACTCGCTGGGCTGCTGGTGCTAGCACTCCTCCTGCTGCTCC |
| | TGTGGAAGATGGGATTCTTCAAACGGGCGAAGCACCCCGAGGCCACCCTGCCCCAGTACC |
| | ATGCGGTGAAGATTCCTCGGGAACACCGACAGCAGTTCAAGGAGGAGAAGACGGGCACCA |
| | TCCTGAGGAACAACTGGGGCAGCCCCCGCCGGGAGGGCCCGGATGCACACCCCATCCTGG |
| | CTGCTGACGGGCATCCCGAGCTGGGCCCCGATGGGCATCCAGGGCCAGGCACCGCCTAGG |
| | TTCCCATGTCCCAGCCTCCCCTGTGGCTCCCCTCCATCCCTTCCCCAGAGATGGCTCCTT |
| | GGGATGAAGAGGGTAGAGTGGGCTGCTGGTGTCGCATCAAGATTTGGCAGGATCGGCTTC |
| | CTCATGGGCACAGACCTC |

| | | |
|---|---|---|
| ORF Start: ATG at 57 | | ORF Stop: TAG at 1317 |
| SEQ ID NO: 202 | 420 aa | MW at 45990.1 kD |

| | |
|---|---|
| NOV36h, | MAGARSRDPWCASGICYLFGSLLVELLFSRAVAFNLDVMGALRKEGEPGSLFGFSVALHR |
| CG56054-09 | |
| Protein Sequence | QLQPRPQSWLLVGAPQALALPGQQANRTCGLFACPLSLEETDCYRVDIDQGADMQKESKE |
| | NQWLGVSVRSQGPGCKIVTCAHRYEARQRVDQTLETRDMICRCFVLSQDLAIRDELDGGE |
| | WKFCEGRPQGHEQFGFCQQGTAAAFSPDSHYLLFCAPGTYNWKGTARVELCAQGSADLAH |
| | LDDGPYEAGGEKEQDPRLIPVPANSTFLEEYSAVKSLEVIVRANITVKSSIKNLMLRDAS |
| | TVIPVMVYLDPMAVVAECVPWWVILLAVLAGLLVLALLVLLLWKMGFFKRAKHPEATVPQ |
| | YHAVKIPREDRQQFKEEKTGTILRNNWGSPRREGPDAHPILAADGHPELGPDGHPGPGTA |

| | |
|---|---|
| SEQ ID NO: 203 | 3595 bp |

| | |
|---|---|
| NOV36i, | TTGGGGCGTGCGAGATTTCCCTTGCATTCGCTGGGAGCTCGCGCAGGGATCGTCCCATGG |
| CG56054-10 | |
| DNA Sequence | CCGGGGCTCGGAGCCGCGACCCTTGGGGGCCCTCCGGGATTTGCTACCTTTTTGGCTCCC |
| | TGCTCGTCGAACTGCTCTTCTCACGGGCTGTCGCCTTCAATCTGGACGTGATGGGTGCCT |
| | TGCGCAAGGAGGGCCAGCCAGGCAGCCTCTTCGGCTTCTCTGTGGCCCTGCACCGGCAGT |
| | TGCAGCCCCGACCCCAGAGCTGGCTGCTGGTGGCTGCTCCCCAGGCCCTGGCTCTTCCTG |
| | GGCAGCAGGCGAATCGCACTGGAGGCCTCTTCGCTTGCCCGTTGAGCCTGGAGGACACTG |
| | ACTGCTACACAGTGGACATCCACCAGGGAGCTGATATGCAAAAGGAAAGCAAGGAGAACC |
| | AGTGGTTCGGAGTCAGTGTTCGGAGCCAGGGGCCTGGGGGCAAGATTGTTACCTGTGCAC |
| | ACCGATATGAGGCAAGGCACCGAGTGGACCACATCCTGGAGACGCGGGATATGATTGGTC |
| | GCTGCTTTGTGCTCAGCCAGGACCTGGCCATCCGGGATGAGTTGGATGGTGGGGAATGGA |
| | AGTTCTGTGAGGGACGCCCCCAAGGCCATGAACAATTTGGGTTCTGCCAGCAGGGCACAG |
| | CTGCCCCCTTCTCCCCTCATAGCCACTACCTCCTCTTTGGGGCCCCAGGAACCTATAATT |
| | GGAAGGGCACGCCCAGGGTGGAGCTCTGTGCACAGGCCTCAGCGGACCTGGCACACCTGG |
| | ACGACGGTCCCTACGAGGCGGGGGGAGAGAAGGAGCACGACCCCCGCCTCATCCCGGTCC |
| | CTGCCAACAGCTACTTTGGCTTCTCTATTGACTCGGGGAAAGGTCTGGTGCGTGCAGAAG |
| | AGCTGAGCTTTGTGGCTGGAGCCCCCCGCGCCAACCACAAGGGTGCTGTGGTCATCCTGC |
| | GCAAGGACAGCGCCAGTCGCCTGGTGCCCGAGGTTATGCTGTCTGGGGAGCGCCTGACCT |
| | CCGGCTTTGGCTACTCACTGGCTGTGGCTGACCTCAACAGTGATGGCTGGCCAGACCTGA |
| | TAGTGGGTGCCCCCTACTTCTTTGAGCGCCAAGAAGAGCTGGGGGGTGCTGTGTATGTGT |
| | ACTTGAACCAGGGGGGTCACTGGGCTGCGATCTCCCCTCTCCGCCTCTCCGGCTCCCCTG |
| | ACTCCATGTTCGGGATCAGCCTGGCTGTCCTGGGCGACCTCAACCAAGATGGCTTTCCAG |
| | ATATTGCAGTGGGTGCCCCCTTTGATGGTGATGGGAAAGTCTTCATCTACCATCGGAGCA |
| | GCCTGGGGGTTGTCGCCAAACCTTCACAGGTGCTCGAGGCCGAGCCTGTGGGCATCAAGA |
| | GCTTCGGCTACTCCCTGTCAGGCAGCTTGGATATGGATGGGAACCAATACCCTGACCTGC |
| | TGGTGGGCTCCCTGGCTGACACCGCAGTCCTCTTCAGGGCCAGACCCATCCTCCATGTCT |
| | CCCATGAGGTCTCTATTGCTCCACGAAGCATCGACCTGGAGCAGCCCAACTCTGCTGGCG |
| | GCCACTCGGTCTGTGTGGACCTAAGGGTCTGTTTCAGCTACATTGCAGTCCCCAGCAGCT |
| | ATAGCCCTACTGTGGCCCTGGACTATCTGTTAGATGCGGACACAGACCGGACGCTCCGGG |
| | GCCAGGTTCCCCGTGTGACGTTCCTGAGCCGTAACCTGGAAGAACCCAAGCACCAGGCCT |
| | CGGGCACCGTGTGGCTGAAGCACCAGCATGACCGACTCTGTGGAGACGCCATGTTCCAGC |
| | TCCAGGAAAATGTCAAAGACAAGCTTCGGGCCATTGTAGTGACCTTGTCCTACACTCTCC |
| | AGACCCCTCGGCTCCGGCGACAGGCTCCTGGCCAGGGGCTGCCTCCAGTGGCCCCCATCC |
| | TCAATGCCCACCAGCCCAGCACCCAGCCGGCAGAGATCCACTTCCTGAAGCAAGGCTGTG |
| | GTGAAGACAAGATCTGCCAGAGCAATCTGCAGCTGGTCCACGCCCGCTTCTGTACCCGGG |
| | TCAGCGACACGGAATTCCAACCTCTGCCCATGGATGTGGATGGAACAACAGCCCTGTTTG |
| | CACTGAGTGGGCAGCCAGTCATTGGCCTGGACCTGATGGTCACCAACCTGCCATCGGACC |
| | CAGCCCAGCCCCAGGCTGATCGGGATGATGCCCATGAAGCCCAGCTCCTGGTCATGCTTC |
| | CTGACTCACTGCACTACTCAGCGGTCCGGGCCCTGGACCCTGCGGAGAAGCCACTCTGCC |
| | TGTCCAATGAGAATGCCTCCCATGTTGAGTGTGAGCTGGGGAACCCCATGAAGAGAGGTG |
| | CCCAGGTCACCTTCTACCTCATCCTTACCACCTCCGGGATCAGCATTCACACCACGGAAC |
| | TGGAGGTAGAGCTCCTGTTGGCCACCATCAGTCAGCAGGACCTGCATCCAGTCTCTGCAC |
| | GAGCCCGTGTCTTCATTGAGCTGCCACTGTCCATTGCAGGAATGGCCATTCCCCAGCAAC |
| | TCTTCTTCTCTGGTGTCGTGACGGGCGAGAGAGCCATGCAGTCTGACCGGGATGTGGGCA |
| | GCAAGGTCAAGTATGAGGTCACGGTTTCCAACCAAGGCCAGTCGCTCAGAACCCTGGCCT |
| | CTGCCTTCCTCAACATCATGTGCCCTCATGAGATTGCCAATGGGAAGTGGTTCCTGTACC |
| | CAATGCAGGTTGAGCTCGACGGCGCGCAGGGGCCTCGGCAGAAAGGGCTTTGCTCTCCCA |
| | GGCCCAACATCCTCCACCTGCATGTGGACACTAGGGATAGCAGGCGGCGGCAGCTGGAGC |
| | CACCTGAGCAGCAGGAGCCTGGTGAGCGGCAGGAGCCCAGCATGTCCTGGTGGCCACTGT |
| | CCTCTGCTGAGAAGAAGAAAAACATCACCCTGGACTGCGCCCGGGGCACGGCCAACTGTG |
| | TGGTGTTCAGCTGCCCACTCTACAGCTTTGACCGCGCGGCTGTGCTGCATGTCTGGGGCC |
| | GTCTCTGGAACAGCACCTTTCTGGAGGAGTACTCAGCTGTGAAGTCCCTGGAAGTGATTG |
| | TCCGGGCCAACATCACAGTGAAGTCCTCCATAAAGAACTTGATGCTCCGAGATGCCTCCA |
| | CAGTGATCCCAGTGATGGTATACTTGGACCCCATGCCTGTGGTGCCAGAAGCAGTGCCCT |
| | GGTGGGTCATCCTCCTGGCTGTACTGGCTGGGCTGCTGGTGCTAGCACTGCTGGTGCTGC |
| | TCCTGTGGAAGTGTGGCTTCTTCCATCGGAGCAGCCAGAGCTCATCTTTTCCCACCAACT |
| | ATCACCGGGCCTGTCTGGCTGTGCAGCCTTCAGCCATGGAAGTTGGGGGTCCAGGGACTG |
| | TGGGATGGGATTCTTCAAACGGGCGAAGCACCCCGAGGCCACCGTGCCCCAGTACCATGC |
| | GGTGAAGATTCCTCGGGAAGACCGACAGCAGTTCAAGGAGGAGAAGACGGGCACCATCCT |
| | GAGGAACAACTGGGGCAGCCCCCGGCGGGAGCGCCCGGATGCACACCCCATCCTGGCTGC |
| | TGACGGGCATCCCGAGCTGGGCCCCGATGGGCATCCAGGGCCAGGCACCGCCTAG |

| | | |
|---|---|---|
| ORF Start: ATG at 57 | | ORF Stop: TGA at 3423 |
| SEQ ID NO: 204 | 1122 aa | MW at 12352.9 kD |

| | |
|---|---|
| NOV36i, | MAGARSRDPWGASGICYLFGSLLVELLFSRAVAFNLDVMGALRKEGEPGSLFGFSVALHR |
| CG56054-10 | |
| Protein Sequence | QLQPRPQSWLLVGAPQALALPGQQANRTGGLFACPLSLEETDCYRVDIDQGADMQKESKE |
| | NQWLGVSVRSQGPGCKIVTCAHRYEARQRVDQILETRDMIGRCFVLSQDLAIRDELDGGE |
| | WKFCEGRPQGHEQFGFCQQGTAAAFSPDSHYLLFGAPGTYNWKGTARVELCAQGSADLAH |
| | LDDGPYEAGGEKEQDPRLIPVPANSYFGFSIDSGKGLVRAEELSFVAGAPRANHKGAVVI |
| | LRKDSASRLVPEVMLSGERLTSGFGYSLAVADLNSDGWPDLIVGAPYFFERQEELGGAVY |
| | VYLNQGGHWAGISPLRLCGSPDSMFGISLAVLGDLNQDGFPDIAVGAPFDGDGKVFIYHG |
| | SSLGVVAKPSQVLEGEAVGIKSFGYSLSGSLDMDGNQYPDLLVGSLADTAVLFRARPILH |
| | VSHEVSIAPRSIDLEQPNCAGGHSVCVDLRVCFSYIAVPSSYSPTVALDYVLDADTDRRL |
| | RGQVPRVTFLSRNLEEPKHQASGTVWLKHQHDRVCGDAMFQLQENVKDKLRAIVVTLSYS |
| | LQTPRLRRQAPGQGLPPVAPILNAHQPSTQRAEIHFLKQGCGEDKICQSNLQLVHARFCT |
| | RVSDTEFQPLPMDVDGTTALFALSGQPVIGLELMVTNLPSDPAQPQADGDDAHEAQLLVM |
| | LPDSLHYSGVRALDPAEKPLCLSNENASHVECELGNPMKRGAQVTFYLILSTSGISIETT |
| | ELEVELLLATISEQELHPVSARARVFIELPLSIAGMAIPQQLFFSGVVRGERAMQSERDV |
| | GSKVKYEVTVSNQGQSLRTLGSAFLNIMWPHEIANGKWLLYPMQVELEGGQGPGQKGLCS |
| | PRPNILHLDVDSRDRRRRELEPPEQQEPGERQEPSMSWWPVSSAEKKKNITLDCARCTAN |
| | CVVFSCPLYSFDRAAVLHVWGRLWNSTFLEEYSAVKSLEVIVRANITVKSSIKNLMLRDA |
| | STVIPVMVYLDPMAVVAEGVPWWVILLAVLAGLLVLALLVLLLWKCGFFHRSSQSSSFPT |
| | NYHRACLAVQPSAMEVGGPGTVGWDSSNCRSTPRPPCPSTMR |

| | |
|---|---|
| SEQ ID NO: 205 | 1034 bp |

| | |
|---|---|
| NOV36j, | GGAGCGGCGGGCGGGCGGGAGGGCTGGCGGGGCGAACGTCTGGGAGACGTCTGAAAGACC |
| CG56054-11 | |
| DNA Sequence | AACGAGACTTTGCAGACCAGAGACGCCCCTGGGGGCACCTGGGGCTTGGGCCGTGCGAGA |
| | TTTCCCTTGCATTCCCTGGGAGCTCGCGCAGGCATCGTCCCATGGCCGGGGCTCGGAGCC |
| | GCGACCCTTGGGGGGCCTCCGCGATTTGCTACCTTTTTGGCTCCCTGCTCGTCGAACTGC |
| | TCTTCTCACGGGCTGTCGCCTTCAATCTGGACGTGATGGGTGCCTTGCGCAAGGAGGGCG |
| | AGCCAGGCAGCCTCTTCCGCTTCTCTGTGGCCCTGCACCGGCAGTTGCAGCCCCGACCCC |
| | AGAGCTGGCTGCTGGTGGGTGCTCCCCAGGCCCTGGCTCTTCCTGGGCAGCAGGCGAATA |
| | GCACTGGAGGCCTCTTCGCTTGCCCGTTGAGCCTGGAGGAGACTGACTGCTACAGAGTGG |
| | ACATCGACCAGGGAGCTGATATGCAAAAGGAAAGCAAGGAGAACCAGTGGTTGGGAGTCA |
| | GTGTCCTCTGCTGAGAAGAAGAAAAACATCACCCTGCACTGCGCCCGGGGCACGCCCAAC |
| | TGTGTGGTGTTCAGCTGCCCACTCTACACCTTTGACCGCGCGGCTGTGCTGCATGTCTGG |
| | GGCCGTCTCTGGAACAGCACCTTTCTCGAGGAGTACTCAGCTGTGAAGTCCCTCGAAGTG |
| | ATTGTCCGGGCCAACATCACAGTGAAGTCCTCCATAAAGAACTTGATGCTCCGAGATGCC |
| | TCCACAGTGATCCCAGTGATGGTATACTTGCACCCCATCGCTGTGGTGGCAGAAGGAGTG |
| | CCCTGGTCGGTCATCCTCCTGGCTGTACTGGCTGGGCTGCTGGTGCTAGCACTGCTGGTG |
| | CTGCTCCTGTGGAAGTGTGGCTTCTTCCATCGGAGCAGCCAGAGCTCATCTTTTCCCACC |
| | AACTATCACCGGGCCTGTCTGGCTGTGCAGCCTTCAGCCATGGAAGTTGGGGGTCCAGGG |
| | ACTGTGGGGTAACT |

| | | |
|---|---|---|
| ORF Start: ATG at 162 | | ORF Stop: TGA at 552 |
| SEQ ID NO: 206 | 130 aa | MW at 14098.0 kD |

| | |
|---|---|
| NOV36j, | MAGARSRDPWGASGICYLFGSLLVELLFSRAVAFNLDVMGALRKEGEPGSLFGFSVALHR |
| CG56054-11 | |
| Protein Sequence | QLQPRPQSWLLVGAPQALALPGQQANRTGGLFACPLSLEETDCYRVDIDQGADMQKESKE |
| | NQWLGVSVLC |

| | |
|---|---|
| SEQ ID NO: 207 | 3972 bp |

| | |
|---|---|
| NOV36k, | GGAGCGGCGGGCGGGCGGGAGGGCTGGCGGGGCGAACGTCTGGGAGACGTCTGAAAGACC |
| CG56054-12 | |
| DNA Sequence | AACGAGACTTTGGAGACCAGAGACGCGCCTGGCGGGACCTGGGGCTTGGGCCGTGCCAGA |
| | TTTCCCTTGCATTCGCTGGGAGCTCGCGCAGGGATCGTCCCATGGCCGGGGCTCGGACCC |
| | GCGACCCTTGGGGGGCCTCCGOGATTTGCTACCTTTTTGGCTCCCTGCTCGTCGAACTGC |
| | TCTTCTCACGGGCTGTCGCCTTCAATCTGGACGTGATCGGTGCCTTCCGCAAGGAGCGCG |
| | AGCCAGGCAGCCTCTTCGGCTTCTCTGTGGCCCTCCACCGGCAGTTGCAGCCCCGACCCC |
| | AGAGCTGGCTGCTCCTGGGTGCTCCCCAGGCCCTGGCTCTTCCTGGGCAGCACGCGAATC |
| | GCACTGGAGGCCTCTTCGCTTCCCCGTTGAGCCTGGAGGAGACTGACTGCTACAGAGTGG |
| | ACATCGACCACGGAGCTGATATGCAAAAGGAAAGCAAGGAGAACCAGTGGTTGGGAGTCA |
| | GTGTTCGGAGCCAGGGGCCTGGGGGCAAGATTGTTACCTGTGCACACCGATATGAGGCAA |
| | GGCAGCGAGTGGACCAGATCCTGGAGACGCGGGATATGATTGGTCGCTGCTTTGTCCTCA |
| | GCCAGGACCTGGCCATCCGGGATGAGTTGGATGGTGGGGAATGGAAGTTCTGTGAGGGAC |
| | GCCCCCAAGGCCATGAACAATTTGGGTTCTGCCAGCAGGGCACAGCTGCCGCCTTCTCCC |
| | CTGATAGCCACTACCTCCTCTTTGGGGCCCCAGGAACCTATAATTGGAAGGGGTTGCTTT |
| | TTGTGACCAACATTGATAGCTCAGACCCCGACCAGCTGGTGTATAAAACTTTGGACCCTG |
| | CTGACCGGCTCCCAGGACCAGCCGGAGACTTGGCCCTCAATAGCTACTTAGGCTTCTCTA |
| | TTCACTCGGGGAAAGGTCTGGTCCGTGCAGAAGAGCTGAGCTTTGTGGCTGGAGCCCCCC |
| | GCGCCAACCACAAGGGTGCTGTGGTTATCCTGCGCAAGGACAGCGCCAGTCGCCTCGTGC |
| | CCGAGGTTATGCTGTCTGGGGAGCGCCTGACCTCCGGCTTTGGCTACTCACTGGCTGTGG |
| | CTGACCTCAACAGTGATGGCTGGCCAGACCTGATAGTGGGTGCCCCCTACTTCTTTGAGC |
| | GCCAAGAAGAGCTGGGCCGTGCTGTGTATGTGTACTTGAACCAGGGGGGTCACTGGGCTG |
| | GGATCTCCCCTCTCCGGCTCTGCGGCTCCCCTGACTCCATGTTCGGGATCAGCCTGGCTG |
| | TCCTGGGGGACCTCAACCAAGATGCCTTTCCAGATATTGCAGTCGGTGCCCCCTTTGATG |
| | GTGATGGGAAAGTCTTCATCTACCATGGGAGCACCCTGGGGGTTGTCGCCAAACCTTCAC |
| | AGGTGCTGGAGGGCGAGGCTGTGGGCATCAAGAGCTTCGGCTACTCCCTGTCAGGCAGCT |
| | TGGATATGGATGGGAACCAATACCCTGACCTGCTGGTGGGCTCCCTGGCTGACACCGCAG |
| | TGCTCTTCAGGGCCAGACCCATCCTCCATGTCTCCCATGAGGTCTCTATTGCTCCACGAA |
| | GCATCGACCTGGACCACCCCAACTCTGCTGCCCCCCACTCGGTCTCTGTCGACCTAAGGG |
| | TCTGTTTCAGCTACATTGCAGTCCCCAGCAGCTATAGCCCTACTGTGGCCCTGGACTATG |
| | TGTTAGATGCGGACACAGACCCGAGGCTCCGGGCCCAGGTTCCCCCTGTGACGTTCCTGA |
| | GCCGTAACCTGCAAGAACCCAAGCACCAGGCCTCGGGCACCGTGTGGCTCAAGCACCAGC |
| | ATGACCGAGTCTGTGCAGACCCCATGTTCCAGCTCCAGGAAAATGTCAAAGACAACCTTC |
| | GGGCCATTGTAGTGACCTTGTCCTACAGTCTCCAGACCCCTCGGCTCCGGCGACAGGCTC |
| | CTGGCCAGGGGCTGCCTCCAGTGCCCCCCATCCTCAATGCCCACCAGCCCACCACCCAGC |
| | GGGCAGAGATCCACTTCCTGAAGCAACGCTCTGGTGAACACAAGATCTGCCAGAGCAATC |
| | TGCAGCTGGTCCACGCCCCCTTCTGTACCCGGGTCAGCGACACGGAATTCCAACCTCTGC |
| | CCATGGATGTGGATGGAACAACACCCCTGTTTGCACTCAGTGGGCAGCCAGTCATTGGCC |
| | TGGAGCTCATGGTCACCAACCTGCCATCGGACCCACCCCAGCCCCAGGCTGATGGCCATG |
| | ATGCCCATGAAGCCCAGCTCCTGCTCATGCTTCCTGACTCACTGCACTACTCAGGGGTCC |
| | GGGCCCTGGACCCTGCGGAGAAGCCACTCTGCCTGTCCAATGAGAATGCCTCCCATCTTG |
| | AGTGTGAGCTCGGGAACCCCATGAAGACAGGTGCCCAGGTCACCTTCTACCTCATCCTTA |
| | GCACCTCCGGGATCAGCATTGAGACCACGGAACTGGAGGTAGAGCTCCTGTTGGCCACGA |
| | TCAGTGACCAGGAGCTGCATCCACTCTCTGCACCAGCCCCTCTCTTCATTGAGCTGCCAC |
| | TGTCCATTGCAGGAATGGCCATTCCCCAGCAACTCTTCTTCTCTGGTGTGGTGAGGGGCG |
| | AGAGAGCCATGCAGTCTCAGCGGGATGTGCGCAGCAAGCTCAACTATGAGGTCACGGTTT |
| | CCAACCAACGCCAGTCCCTCACAACCCTGCGCTCTGCCTTCCTCAACATCATGTGGCCTC |
| | ATGAGATTGCCAATGGGAAGTCGTTGCTGTACCCAATGCAGGTTGAGCTCGAGGGCGGGC |
| | AGGGGCCTGGCCAGAAAGGGCTTTCCTCTCCCAGGCCCAACATCCTCCACCTGGATGTGG |
| | ACAGTAGGCATAGGAGGCGGCGGGACCTGGACCCACCTCAGCAGCAGGACCCTGGTGAGC |
| | GGCAGGAGCCCACCATGTCCTGGTGGCCAGTGTCCTCTCCTCAGAAGAACAAAAACATCA |
| | CCCTGGACTGCGCCCGGGGCACGGCCAACTGTGTGGTGTTCAGCTGCCCACTCTACAGCT |
| | TTGACCGCGCGGCTGTGCTGCATCTCTGGCGCCGTCTCTGGAACAGCACCTTTCTGGAGG |
| | AGTACTCAGCTGTGAAGTCCCTGGAAGTGATTGTCCCGCCCAACATCACAGTGAAGTCCT |
| | CCATAAAGAACTTGATGCTCCGAGATGCCTCCACAGTGATCCCAGTGATGGTATACTTGG |
| | ACCCCATCCCTGTGGTGGCAGAACGAGTGCCCTGGTGGGTCATCCTCCTCGCTGTACTGG |
| | CTGGCCTGCTGGTGCTAGCACTCCTGGTCCTGCTCCTGTGGAAGATGGGATTCTTCAAAC |
| | GGGCGAAGCACCCCCCGGCGGGAGGGCCCGGATGCACACCCCATCCTGGCTGCTGACGGG |
| | CATCCCGAGCTGGGCCCCGATGGGCATCCAGGGCCACCCACCGCCTAGGTTCCCATGTCC |
| | CAGCCTCGCCTGTGGCTGCCCTCCATCCCTTCCCCACACATGGCTCCTTGGGATGAAGAG |
| | GGTAGAGTGGGCTGCTGGTGTCCCATCAAGATTTGGCAGGATCGGCTTCCTCAGGGGCAC |
| | AGACCTCTCCCACCCACAAGAACTCCTCCCACCCAACTTCCCCTTAGAGTCCTGTGAGAT |
| | GAGAGTGGGTAAATCAGCCACAGGGCCATGGGGTAGGGTGAGAAGGGCAGCGGTGTCCTG |
| | ATGCAAAGGTGGCGAGAAGGGATCCTAATCCCTTCCTCTCCCATTCACCCTGTGTAACAG |
| | GACCCCAAGGACCTGCCTCCCCCGAAGTGCCTTAACCTAGAGGGTCGGCGAGGAGGTTGT |
| | GTCACTGACTCAGGCTGCTCCTTCTCTACTTTCCCCTCTCATCTGACCTTAGTTTGCTGC |
| | CATCAGTCTAGTGGTTTCCTCGTTTCGTCTATTTATTAAAAAATATTTCAGAACAAAAAA |
| | AAAAAAAAAAAA |

| | | |
|---|---|---|
| ORF Start: ATG at 162 | | ORF Stop: TGA at 3414 |
| SEQ ID NO: 208 | 1084 aa | MW at 118234.7 kD |

| | |
|---|---|
| NOV36k, | MAGARSRDPWGASGICYLFGSLLVELLFSRAVAFNLDVMGALRKEGEPGSLFGFSVALHR |
| CG56054-12 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCECRPQGHEQFGFCQQGTAAAFSPDSHYLLFGAPGTYNWKGLLPVTNIDSSDPDQLV |
| | YKTLDPADRLPGPAGDLALNSYLGFSIDSGKGLVRAEELSFVAGAPRANHKGAVVILRKD |
| | SASRLVPEVMLSGERLTSGFGYSLAVADLNSDGWPDLIVGAPYFFERQEELGGAVYVYLN |
| | QGGHWAGISPLRLCGSPDSMFGISLAVLGDLNQDGFPDIAVGAPFDGDGKVFIYHGSSLG |
| | VVAKPSQVLEGEAVGIKSFCYSLSGSLDMDGNQYPDLLVGSLADTAVLFRARPILHVSHE |
| | VSIAPRSIDLEQPNCAGGHSVCVDLRVCFSYIAVPSSYSPTVALDYVLDADTDRRLRGQV |
| | PRVTFLSRNLEEPKHQASGTVWLKHQHDRVCGDAMFQLQENVKDKLRAIVVTLSYSLQTP |
| | RLRRQAPGQGLPPVAPILNAHQPSTQRAEIHFLKQGCGEDKICQSNLQLVHARFCTRVSD |
| | TEFQPLPMDVDGTTALFALSGQPVIGLELMVTNLPSDPAQPQADGDDAHEAQLLVMLPDS |
| | LHYSGVRALDPAEKPLCLSNENASIVECELGNPMKRGAQVTFYLILSTSGISIETTELEV |
| | ELLLATISEQELHPVSARARVFIELPLSIAGMAIPQQLFFSGVVRGERAMQSERDVGSKV |
| | KYEVTVSNQGQSLRTLGSAFLNIMWPHEIANGKWLLYPMQVELEGGQGPGQKGLCSPRPN |
| | ILHLDVDSRDRRRRELEPPEQQEPGERQEPSMSWWPVSSAEKKKNITLDCARGTANCVVF |
| | SCPLYSFDRAAVLHVWGRLWNSTFLEEYSAVKSLEVIVRANITVKSSIKNLMLRDASTVI |
| | PVMVYLDPMAVVAEGVPWWVILLAVLAGLLVLALLVLLLWKMGFFKRAKHPPAGGPGCTP |
| | HPGC |

| | |
|---|---|
| SEQ ID NO: 209 | 3583 bp |

| | |
|---|---|
| NOV361, | TTGGGGCGTGCGAGATTTCCCTTGCATTCGCTCGGAGCTCGCGCAGGGATCGTCCCATGG |
| CG56054-13 | |
| DNA Sequence | CCGGGGCTCGGAGCCCCGACCCTTGGGGGGCCTCCGGGATTTCCTACCTTTTTGCCTCCC |
| | TGCTCGTCGAACTGCTCTTCTCACGGGCTGTCGCCTTCAATCTGGACGTGATGGGTGCCT |
| | TGCGCAAGGAGCGCGAGCCAGGCAGCCTCTTCGGCTTCTCTGTGGCCCTGCACCGGCAGT |
| | TGCAGCCCCGACCCCAGAGCTGCCTGCTGGTGGGTGCTCCCCAGGCCCTGGCTCTTCCTG |
| | GGCAGCAGGCGAATCGCACTGGAGGCCTCTTCCCTTGCCCGTTGACCCTGGAGGAGACTG |
| | ACTGCTACAGAGTGGACATCGACCAGGGAGCTGATATGCAAAAGGAAAGCAAGGAGAACC |
| | AGTGGTTGGCAGTCACTGTTCGGAGCCAGGGGCCTCGGGGCAAGATTGTTACCTGTGCAC |
| | ACCGATATCACGCAAGGCAGCGAGTGGACCAGATCCTCGACACCCGGGATATCATTCGTC |
| | GCTGCTTTGTGCTCAGCCAGGACCTGGCCATCCGGGATGAGTTGGATGGTGGGGAATGGA |
| | AGTTCTGTGAGGGACGCCCCCAACGCCATGAACAATTTGGGTTCTGCCAGCAGGGCACAG |
| | CTGCCGCCTTCTCCCCTGATAGCCACTACCTCCTCTTTGGGGCCCCAGGAACCTATAATT |
| | GGAAGGGGTTGCTTTTTGTGACCAACATTGATAGCTCAGACCCCGACCAGCTGGTGTATA |
| | AAACTTTGGACCCTCCTGACCGGCTCCCAGGACCAGCCGGAGACTTGCCCCTCAATAGCT |
| | ACTTAGGCTTCTCTATTGACTCGGGGAAAGGTCTGGTGCGTGCAGAAGAGCTGACCTTTG |
| | TGGCTGGAGCCCCCCCCGCCAACCACAAGGGTGCTGTGCTCATCCTGCGCAAGCACAGCG |
| | CCAGTCGCCTGGTGCCCGAGGTTATGCTGTCTGGGGAGCGCCTGACCTCCGGCTTTGGCT |
| | ACTCACTGGCTGTGGCTGACCTCAACAGTGATGGCTGCCCAGACCTGATAGTGGGTGCCC |
| | CCTACTTCTTTGAGCGCCAAGAACAGCTGGCCGGTGCTGTGTATGTGTACTTGAACCAGG |
| | GGGGTCACTCGGCTGCGATCTCCCCTCTCCGGCTCTGCGGCTCCCCTGACTCCATGTTCG |
| | GGATCAGCCTGGCTGTCCTGGCGGACCTCAACCAAGATGGCTTTCCACATATTGCAGTGG |
| | GTGCCCCCTTTGATGGTGATCGGAAAGTCTTCATCTACCATGGGAGCAGCCTGGGGCTTG |
| | TCGCCAAACCTTCACAGGTGCTGGACGGCGAGGCTGTGGGCATCAAGAGCTTCGGCTACT |
| | CCCTGTCAGGCAGCTTGCATATGGATGCCAACCAATACCCTGACCTGCTGGTCGGCTCCC |
| | TGGCTGACACCGCAGTGCTCTTCAGGGCCACACCCATCCTCCATGTCTCCCATCAGGTCT |
| | CTATTGCTCCACGAAGCATCCACCTGGAGCAGCCCAACTGTGCTGGCGGCCACTCGGTCT |
| | GTGTGGACCTAAGGGTCTCTTTCAGCTACATTGCAGTCCCCAGCAGCTATAGCCCTACTG |
| | TGGCCCTGCACTATGTGTTAGATGCCGACACAGACCGGAGGCTCCGGGGCCAGGTTCCCC |
| | GTGTGACGTTCCTCACCCGTAACCTGGAAGAACCCAAGCACCAGGCCTCGCCCACCGTGT |
| | GGCTGAAGCACCAGCATGACCCAGTCTCTGGAGACGCCATGTTCCAGCTCCAGGAAAATG |
| | TCAAAGACAAGCTTCGCGCCATTCTAGTGACCTTGTCCTACAGTCTCCAGACCCCTCGGC |
| | TCCGGCGACAGGCTCCTGGCCAGGGGCTGCCTCCAGTGGCCCCCATCCTCAATGCCCACC |
| | AGCCCAGCACCCAGCGGGCAGAGATCCACTTCCTGAAGCAAGGCTGTGGTGAAGACAAGA |
| | TCTGCCAGAGCAATCTGCAGCTGGTCCACGCCCGCTTCTGTACCCGGGTCAGCGACACGG |
| | AATTCCAACCTCTGCCCATGGATCTGGATGGAACAACAGCCCTGTTTGCACTGAGTGGGC |
| | AGCCAGTCATTGGCCTGGAGCTGATGGTCACCAACCTGCCATCGGACCCAGCCCAGCCCC |
| | AGGCTGATGGGGATGATGCCCATGAAGCCCAGCTCCTGGTCATGCTTCCTGACTCACTGC |
| | ACTACTCAGGGGTCCGGGCCCTGGACCCTGCCGAGAAGCCACTCTGCCTGTCCAATGAGA |
| | ATGCCTCCCATGTTCACTGTGAGCTGGGGAACCCCATGAAGAGAGGTGCCCAGGTCACCT |
| | TCTACCTCATCCTTAGCACCTCCGGGATCAGCATTGAGACCACGGAACTGGAGGTAGAGC |
| | TGCTGTTGGCCACGATCAGTGAGCAGGAGCTGCATCCAGTCTCTGCACGAGCCCGTGTCT |
| | TCATTGAGCTGCCACTGTCCATTGCAGGAATGGCCATTCCCCAGCAACTCTTCTTCTCTG |
| | GTGTGGTGAGGGGCGAGAGAGCCATGCAGTCTGACCGGGATGTGGGCACCAAGGTCAAGT |
| | ATGAGGTCACGGTTTCCAACCAAGGCCAGTCGCTCAGAACCCTGGGCTCTGCCTTCCTCA |
| | ACATCATGTGGCCTCATGAGATTGCCAATGGGAAGTGGTTGCTGTACCCAATGCAGGTTG |
| | AGCTGGAGGGCGGGCAGGGGCCTGGGCAGAAAGGGCTTTGCTCTCCCAGGCCCAACATCC |
| | TCCACCTGGATGTGGACAGTAGGGATAGGAGCCGGCGGGAGCTGGAGCCACCTGAGCAGC |
| | AGGAGCCTGGTGAGCGGCAGGAGCCCAGCATCTCCTGGTGGCCAGTGTCCTCTGCTGAGA |
| | AGAAGAAAAACATCACCCTGGACTGCCCCCGGGGCACGGCCAACTGTGTGGTGTTCAGCT |
| | GCCCACTCTACAGCTTTGACCGCGCGCCTGTGCTGCATGTCTGGGGCCGTCTCTGGAACA |
| | GCACCTTTCTGGAGGAGTACTCAGCTGTGAAGTCCCTGGAAGTGATTGTCCGGGCCAACA |
| | TCACAGTCAAGTCCTCCATAAAGAACTTGATGCTCCGAGATGCCTCCACAGTGATCCCAG |
| | TGATCGTATACTTGGACCCCATGGCTGTGGTGGCAGAAGGAGTGCCCTGGTGGGTCATCC |
| | TCCTGGCTGTACTGGCTGGGCTGCTGGTGCTAGCACTGCTGGTGCTGCTCCTGTGGAAGT |
| | GTGGCTTCTTCCATCGGAGCAGCCAGAGCTCATCTTTTCCCACCAACTATCACCGGGCCT |
| | GTCTGGCTGTGCAGCCTTCACCCATGGAAGTTGGGGGTCCAGGGACTGTGGGATGGGATT |
| | CTTCAAACCGGCCAAGCACCCCGAGGCCACCGTGCCCCACTACCATGCGGTGAAGATTCC |
| | TCGGGAAGACCGACAGCACTTCAAGGAGGAGAAGACGCGCACCATCCTGAGAAACAACTG |
| | GGGCAGCCCCCGGCGCGAGGGCCCGCATGCACACCCCATCCTGGCTGCTGACGGGCATCC |
| | CGACCTGGGCCCCGATGGGCATCCAGGGCCAGGCACCGCCTAG |

| | | |
|---|---|---|
| ORF Start: ATG at 57 | | ORF Stop: TGA at 3411 |
| SEQ ID NO: 210 | 1118 aa | MW at 121969.6 kD |

| | |
|---|---|
| NOV361, | MAGARSRDPWGASGICYLFGSLLVELLFSRAVAFNLDVMGALRKEGEPGSLFGFSVALHR |
| CG56054-13 | |
| Protein Sequence | QLQPRPQSWLLVGAPQALALPGQQANRTGGLFACPLSLEETDCYRVDIDQGADMQKESKE |
| | NQWLGVSVRSQCPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGLLFVTNIDSSDPDQLV |
| | YKTLDPADRLPGPACDLALNSYLGFSIDSGKGLVRAEELSFVACAPRANHKGAVVILRKD |
| | SASRLVPEVMLSCERLTSGFGYSLAVADLNSDGWPDLIVGAPYFFERQEELCGAVYVYLN |
| | QGGHWAGISPLRLCGSPDSMFCTSLAVLGDLNQDGFPDTAVGAPFDGDGKVFIYHGSSLG |
| | VVAKPSQVLEGEAVCTKSFGYSLSGSLDMDGNQYPDLLVGSLADTAVLFRARPILHVSHE |
| | VSIAPRSIDLEQPNCAGGHSVCVDLRVCFSYTAVPSSYSPTVALDYVLDADTDRRLRGQV |
| | PRVTFLSRNLEEPKHQASGTVWLKHQHDRVCGDAMFQLQENVKDKLRAIVVTLSYSLQTP |
| | RLRRQAPGQGLPPVAPILNAHQPSTQRAEIHFLKQGCGEDKICQSNLQLVHARFCTRVSD |
| | TEFQPLPMDVDGTTALFALSGQPVIGLELMVTNLPSDPAQPQADGDDAHEAQLLVMLPDS |
| | LHYSGVRALDPAEKPLCLSNENASHVECELGNPMKRGAQVTFYLILSTSGISIETTELEV |
| | ELLLATISEQELHPVSARARVFIELPLSTAGMATPQQLFFSGVVRGERAMQSERDVGSKV |
| | KYEVTVSNQGQSLRTLCSAFLNIMWPHETANGKWLLYPMQVELEGGQGPGQKGLCSPRPN |
| | ILHLDVDSRDRRRRELEPPEQQEPGERQEPSMSWWPVSSAEKKKNITLDCARGTANCVVF |
| | SCPLYSFDRAAVLHVWGRLWNSTFLEEYSAVKSLEVIVRANITVKSSIKNLMLRDASTVI |
| | PVMVYLDPMAVVAEGVPWWVILLAVLAGLLVLALLVLLLWKCGFFHRSSQSSSFPTNYHR |
| | ACLAVQPSANEVGGPGTVGWDSSNGRSTPRPPCPSTMR |

| | |
|---|---|
| SEQ ID NO: 211 | 3938 bp |

| | |
|---|---|
| NOV36m, | TTGGGGCGTGCGAGATTTCCCTTGCATTCGCTGGGAGCTCGCGCAGGGATCGTCCCATG |
| CG56054-14 | |
| DNA Sequence | GCCGGGGCTCGGAGCCCCGACCCTTGGGGGGCCTCCGGGATTTGCTACCTTTTTGGCTC |
| | CCTGCTCGTCGAACTGCTCTTCTCACGGGCTGTCGCCTTCAATCTGGACGTGATGCGTG |
| | CCTTGCGCAAGGAGGGCGAGCCAGGCAGCCTCTTCGGCTTCTCTGTGGCCCTGCACCGG |
| | CAGTTGCAGCCCCGACCCCAGAGCTGGCTGCTGGTGGGTGCTCCCCAGGCCCTGGCTCT |
| | TCCTGGGCAGCAGGCGAATCGCACTGGAGGCCTCTTCGCTTGCCCGTTGAGCCTGGAGG |
| | AGACTGACTGCTACAGAGTGGACATCGACCAGGGAGCTGATATGCAAAAGGAAAGCAAG |
| | GAGAACCAGTGGTTGGGAGTCAGTGTTCGGAGCCAGGGGCCTGGGGGCAAGATTGTTAC |
| | CTGTGCACACCGATATGAGGCAAGGCAGCGAGTGGACCAGATCCTGGAGACGCGGGATA |
| | TGATTGGTCGCTGCTTTGTGCTCAGCCAGGACCTGGCCATCCGGGATGAGTTGGATGGT |
| | GGGGAATGGAAGTTCTGTGAGGGACGCCCCCAAGGCCATGAACAATTTGGGTTCTGCCA |
| | GCAGGGCACAGCTGCCGCCTTCTCCCCTGATAGCCACTACCTCCTCTTTGGGGCCCCAG |
| | GAACCTATAATTGGAACGGCACGGCCAGGGTGGAGCTCTGTGCACAGGGCTCAGCGGAC |
| | CTGGCACACCTGGACGACGGTCCCTACGAGGCGGGGGGACAGAAGGAGCAGGACCCCCG |
| | CCTCATCCCGGTCCCTGCCAACAGCTACTTTGGCTTCTCTATTGACTCGGGGAAAGGTC |
| | TGGTGCGTGCAGAAGAGCTGAGCTTTGTGGCTGGAGCCCCCCGCGCCAACCACAAGGGT |
| | GCTGTGGTTATCCTGCGCAAGGACAGCGCCAGTCGCCTGGTGCCCGAGGTTATGCTGTC |
| | TGGGGAGCGCCTGACCTCCGGCTTTGGCTACTCACTGGCTGTGGCTGACCTCAACAGTG |
| | ATGGCTGGCCAGACCTGATAGTGGGTGCCCCCTACTTCTTTGAGCGCCAAGAAGAGCTG |
| | GGGGGTGCTGTGTATGTGTACTTGAACCAGGCGGGTCACTGGGCTGGGATCTCCCCTCT |
| | CCGGCTCTGCGGCTCCCCTGACTCCATGTTCGGGATCAGCCTGGCTGTCCTGGGGGACC |
| | TCAACCAAGATGGCTTTCCAGATATTGCAGTGGGTGCCCCCTTTGATGGTGATGGGAAA |
| | GTCTTCATCTACCATGGGAGCAGCCTGGGGGTTGTCGCCAAACCTTCACAGGTGCTGGA |
| | GGGCGAGGCTGTGGGCATCAAGAGCTTCGGCTACTCCCTGTCAGGCAGCTTGGATATGG |
| | ATGGGAACCAATACCCTGACCTGCTGGTGGGCTCCCTGGCTGACACCGCAGTGCTCTTC |
| | AGGGCCAGACCCATCCTCCATGTCTCCCATGAGGTCTCTATTGCTCCACGAAGCATCGA |
| | CCTGGAGCAGCCCAACTGTGCTGGCGGCCACTCGGTCTGTGTGGACCTAAGGGTCTGTT |
| | TCAGCTACATTGCAGTCCCCAGCAGCTATAGCCCTACTGTGGCCCTGGACTATGTGTTA |
| | GATGCGGACACAGACCGGAGGCTCCGGGGCCAGGTTCCCCGTGTGACGTTCCTGAGCCG |
| | TAACCTGGAAGAACCCAAGCACCAGGCCTCGGGCACCGTGTGGCTGAAGCACCAGCATG |
| | ACCGAGTCTGTGGAGACGCCATGTTCCAGCTCCAGGAAAATGTCAAAGACAAGCTTCGG |
| | GCCATTGTAGTGACCTTGTCCTACAGTCTCCAGACCCCTCCGCTCCGGCGACAGGCTCC |
| | TGGCCAGGGCCTCCCTCCAGTGCCCCCCATCCTCAATGCCCACCAGCCCAGCACCCAGC |
| | GGGCAGAGATCCACTTCCTGAAGCAAGGCTGTCGTGAAGACAAGATCTCCCAGAGCAAT |
| | CTGCAGCTGGTCCACGCCCGCTTCTGTACCCGGCTCAGCGACACGGAATTCCAACCTCT |
| | GCCCATGGATGTGGATGGAACAACAGCCCTGTTTGCACTGAGTGGCCAGCCAGTCATTG |
| | GCCTGGAGCTGATCGTCACCAACCTGCCATCCCACCCAGCCCAGCCCCAGGCTGATGGG |
| | GATGATGCCCATGAACCCCAGCTCCTGGTCATGCTTCCTGACTCACTGCACTACTCAGG |
| | GCTCCCCGCCCTGCACCCTCCGGAGAAGCCACTCTCCCTGTCCAATCAGAATGCCTCCC |
| | ATGTTGAGTGTGAGCTGGGGAACCCCATGAACAGAGGTGCCCACGTCACCTTCTACCTC |
| | ATCCTTAGCACCTCCGGGATCACCATTGAGACCACGGAACTGGAGGTAGAGCTCCTGTT |
| | GGCCACGATCAGTGAGCAGCAGCTGCATCCACTCTCTGCACCAGCCCGTGTCTTCATTG |
| | AGCTGCCACTGTCCATTGCAGGAATGCCCATTCCCCAGCAACTCTTCTTCTCTGCTGTG |
| | GTGAGGGGCGAGACACCCATGCAGTCTGAGCGGCATGTGGGCAGCAAGGTCAAGTATGA |
| | GGTCACGGTTTCCAACCAAGGCCAGTCGCTCAGAACCCTGGGCTCTGCCTTCCTCAACA |
| | TCATCTGGCCTCATGAGATTGCCAATGGGAAGTCGTTGCTGTACCCAATGCAGGTTGAG |
| | CTGGAGGGCGGGCAGGGCCCTGGGCACAAAGGGCTTTGCTCTCCCAGGCCCAACATCCT |
| | CCACCTCGATGTGGACAGTAGCCATAGCAGGCGCCGGGAGCTGGAGCCACCTGAGCAGC |
| | AGGAGCCTGGTGAGCGGCAGGAGCCCAGCATGTCCTGGTGGCCACTGTCCTCTGCTGAG |
| | AAGAAGAAAAACATCACCCTCGACTGCGCCCGGGGCACGCCCAACTGTGTGGTGTTCAG |
| | CTGCCCACTCTACAGCTTTCACCGCGCGGCTGTGCTGCATGTCTGGGGCCGTCTCTGGA |
| | ACAGCACCTTTCTGGAGGAGTACTCAGCTGTGAAGTCCCTGGAAGTGATTGTCCGGGCC |
| | AACATCACAGTGAAGTCCTCCATAAAGAACTTGATGCTCCGAGATGCCTCCACAGTGAT |
| | CCCAGTCATGGTATACTTGCACCCCATGGCTCTGCTGGCAGAAGGAGTGCCCTGGTGGG |
| | TCATCCTCCTGGCTGTACTGCCTGGGCTGCTCGTGCTAGCACTGCTCGTCCTGCTCCTG |
| | TGGAAGATGGGATTCTTCAAACGGGCGAAGCACCCCGACGCCACCGTGCCCCAGTACCA |
| | TGCGGTGAAAATTCCTCGGGAAGACCCACAGCAGTTCAAGGAGGACAAGACGGGCACCA |
| | CAGGCACCGCCTAGGTTCCCATGTCCCAGCCTGGCCTGTGGCTGCCCTCCATCCCTTCC |
| | CCACAGATGGCTCCTTGGGATGAAGAGGGTAGAGTGGCCTCCTGGTGTCGCATCAAGAT |
| | TTCCCACGATCGGCTTCCTCAGCGCACAGACCTCTCCCCCCACAAGAACTCCTCCCACC |
| | CAACTTCCCCTTAGAGTGCTGTGAGATGAGACTGGGTAAATCACGGACAGGGCCATGGG |
| | GTAGGGTGAGAAGGGCAGGGGTGTCCTGATGCAAACGTGGGCAGAAGGCATCCTAATCC |
| | CTTCCTCTCCCATTCACCCTGTGTAACAGGACCCCAAGCACCTGCCTCCCCGGAAGTGC |
| | CTTAACCTAGAGGGTCCGGGAGGAGGTTGTGTCACTGACTCAGGCTGCTCCTTCTCTAG |
| | TTTCCCCTCTCATCTGACCTTAGTTTGCTGCCATCAGTCTAGTGGTTTCCTCGTTTCGT |
| | CTATTTATTAAAAAATATTTGAGAACAAAAAAAAAAAAAAAAAA |

| | | |
|---|---|---|
| ORF Start: ATG at 57 | | ORF Stop: TGA at 3621 |
| SEQ ID NO: 212 | 1188 aa | WW at 130044.2 kD |

| | |
|---|---|
| NOV36m, | MAGARSRDPWGASGICYLFCSLLVELLFSRAVAFNLDVNGALRKEGEPGSLPGPSVALH |
| CG56054-14 | |
| Protein Sequence | RQLQPRPQSWLLVGAPQALALPGQQANRTGGLFACPLSLEETDCYRVDIDQGADMQKES |
| | KENQWLGVSVRSQGPGGKIVTCAHRYEARQRVDQILETRDMIGRCFVLSQDLAIRDELD |
| | GGEWKFCEGRPQGHEQFGFCQQGTAAAFSPDSHYLLFGAPGTYNWKGTARVELCAQGSA |
| | DLAHLDDGPYEAGGEKEQDPRLIPVPANSYFGFSIDSGKGLVRAEELSFVAGAPRANHK |
| | GAVVILRKDSASRLVPEVMLSGERLTSGFGYSLAVADLNSDGWPDLIVGAPYFFERQEE |
| | LGGAVYVYLNQGGHWAGISPLRLCGSPDSMFCISLAVLGDLNQDGFPDIAVGAPFDGDG |
| | KVFIYHGSSLGVVAKPSQVLEGEAVGIKSFGYSLSGSLDMDCNQYPDLLVGSLADTAVL |
| | FRARPILHVSHEVSIAPRSIDLEQPNCAGGHSVCVDLRVCFSYIAVPSSYSPTVALDYV |
| | LDADTDRRLRGQVPRVTFLSRNLEEPKHQASGTVWLKHQHDRVCGDAMFQLQENVKDKL |
| | RAIVVTLSYSLQTPRLRRQAPGQGLPPVAPILNAHQPSTQRAEIHFLKQGCGEDKICQS |
| | NLQLVHARFCTRVSDTEFQPLPMDVDGTTALFALSGQPVIGLELMVTNLPSDPAQPQAD |
| | GDDAHEAQLLVMLPDSLHYSGVRALDPAEKPLCLSNENASHVECELGNPMKRGAQVTFY |
| | LILSTSGISIETTELEVELLLATISEQELHPVSARARVFIELPLSIAGMAIPQQLFFSG |
| | VVRGERAMQSERDVGSKVKYEVTVSNQGQSLRTLGSAFLNTMWPHEIANGKWLLYPMQV |
| | ELEGGQGPGQKGLCSPRPNILHLDVDSRDRRRRELEPPEQQEPGERQEPSMSWWPVSSA |
| | EKKKNITLDCARGTANCVVFSCPLYSFDRAAVLHVWGRLWNSTFLEEYSAVKSLEVIVR |
| | ANITVKSSIKNLMLRDASTVIPVMVYLDPMAVVAEGVPWWVILLAVLAGLLVLALLVLL |
| | LWKNGFFKRAKHPEATVPQYHAVKIPREDRQQFKEEKTGTILRNNWGSPHPGWAPMGIQ |
| | GQAPPRFPCPSLACGCPPSLPQRWLLGMKRVEWAAGVASRFGRIGFLRAQTSPPTRTPP |
| | TQLPLRVL |

| | |
|---|---|
| SEQ ID NO: 213 | 2471 bp |

| | |
|---|---|
| NOV36n, | TTGGGGCGTCCGAGATTTCCCTTGCATTCCCTGGGAGCTCGCGCAGGGATCGTCCCATGG |
| CG56054-15 | |
| DNA Sequence | CCGGGGCTCGCAGCCGCCACCCTTCGGGGGCCTCCCGGATTTGCTACCTTTTTGGCTCCC |
| | TGCTCGTCGAACTGCTCTTCTCACGGGCTGTCGCCTTCAATCTGCACGTGATGGCTCCCT |
| | TGCGCAAGGAGGGCGAGCCAGGCACCCTCTTCGGCTTCTCTGTGGCCCTGCACCCCCAGT |
| | TGCAGCCCCGACCCCAGAGCTGGCTGCTGGTGGCTCCTCCCCAGGCCCTGGCTCTTCCTG |
| | GGCAGCAGGCCAATCGCACTGGAGCCCTCTTCGCTTGCCCGTTCAGCCTCGAGGAGACTG |
| | ACTGCTACAGAGTGGACATCGACCAGGGAGCTGATATGCAAAAGGAAAGCAAGGAGAACC |
| | AGTGGTTGGGAGTCAGTGTTCGGAGCCAGGGGCCTGGGGGCAAGATTGTTACCTGTGCAC |
| | ACCGATATGAGGCAAGGCAGCGAGTGGACCAGATCCTGGAGACGCGGGATATGATTGGTC |
| | GCTGCTTTGTCCTCAGCCAGGACCTGGCCATCCGGGATGAGTTGGATGGTGGGGAATGGA |
| | AGTTCTGTGAGGCACGCCCCCAAGGCCATGAACAATTTGGGTTCTGCCAGCAGGGCACAG |
| | CTGCCGCCTTCTCCCCTGATAGCCACTACCTCCTCTTTGGGGCCCCAGGAACCTATAATT |
| | GGAAGGGCACGGCCACGGTGGAGCTCTGTGCACACGGCTCAGCGGACCTGGCACACCTGG |
| | ACGACGGTCCCTACGAGGCGGCGGCAGAGAAGGAGCACGACCCCCGCCTCATCCCGGTCC |
| | CTGCCAACAGCTACTTTGGCTTCTCTATTCACTCGGGGAAAGGTCTGGTGCGTCCAGAAG |
| | AGCTGAGCTTTGTGGCTGGAGCCCCCCGCGCCAACCACAAGCGTGCTGTGGTTATCCTGC |
| | GCAAGGACACCGCCAGTCGCCTGGTGCCCGAGGTTATGCTGTCTCGGGAGCGCCTGACCT |
| | CCCGCTTTGGCTACTCACTGGCTGTCGCTGACCTCAACAGTGATGGCTGCCCAGACCTGA |
| | TAGTGGGTGCCCCCTACTTCTTTGAGCGCCAAGAAGACCTCGGGGGTGCTGTGTATGTGT |
| | ACTTGAACCAGGGGGGTCACTGGGCTGGGATCTCCCCTCTCCGCCTCTGCAACTCCCCGC |
| | ACTCCATGTTCGGGATCAGCCTGGCTGTCCTCGCGCACCTCPACCAAGATCGCTTTCCAG |
| | ATATTGCAGTCGGTGCCCCCTTTGATGGTCATGGGAAAGTCTTCATCTACCATGGCAGCA |
| | GCCTGGGGGTTGTCCCCAAACCTTCACAGGTGCTGGAGGGCGAGGCTGTCGGCATCAAGA |
| | GCTTCCGCTACTCCCTGTCAGGCAGCTTGGATATGGATCGCAACCAATACCCTGACCTGC |
| | TGGTCGGCTCCCTGGCTGACACCGCAGTGCTCTTCAGCGCCAGACCCATCCTCCATCTCT |
| | CCCATGAGGTCTCTATTGCTCCACGAAGCATCGACCTGGAGCAGCCCAACTGTGCTGGCG |
| | GCCACTCGGTCTGTGTCGACCTAAGGGTCTGTTTCAGCTACATTGCAGTCCCCACCAGCT |
| | ATAGCCCTACTGTGCCCCTGGACTATGTCTTACATGCGGACACAGACCCGAGGCTCCGGG |
| | GCCAGGTTCCCCGTGTGACGTTCCTGAGCCGTAACCTGGAAGAACCCAAGCACCAGGCCT |
| | CGGGCACCGTGTGGCTGAACCACCAGCATGACCCAGTCTGTGGAGACGCCATGTTCCAGC |
| | TCCAGGAAAATGTCAAAGACAAGCTTCGGCCCATTGTAGTCACCTTCTCCTACAGTCTCC |
| | AGACCCCTCGGCTCCGGCGGCAGCGCCCGGATGCACACCCCATCCTGGCTGCTGACGGGC |
| | ATCCCGAGCTGGGCCCCGATGGGCATCCAGGGCCAGGCACCGCCTAGGTTCCCATGTCCC |
| | AGCCTGCCCTGTGGCTCCCCTCCATCCCTTCCCCAGAGATGGCTCCTTGGGATGAAGAGG |
| | GTAGAGTGGGCTGCTGGTGTCGCATCAAGATTTGGCAGGATCGGCTTCCTCAGGGGCACA |
| | GACCTCTCCCACCCACAAGAACTCCTCCCACCCAACTTCCCCTTAGAGTGCTCTCAGATG |
| | AGAGTCGGTAAATCAGGGACAGCGCCATGGGGTAGGGTGAGAAGGGCAGGGGTGTCCTGA |
| | TGCAAAGGTGGGGAGAAGGGATCCTAATCCCTTCCTCTCCCATTCACCCTGTGTAACAGG |
| | ACCCCAAGGACCTGCCTCCCCGGAAGTGCCTTAACCTAGAGGGTCGGGGAGGAGGTTGTG |
| | TCACTGACTCAGGCTGCTCCTTCTCTAGTTTCCCCTCTCATCTGACCTTAGTTTGCTGCC |
| | ATCAGTCTAGTGGTTTCGTGGTTTCGTCTATTTATTAAAAAATATTTGAGAACAAAAAAA |
| | AAAAAAAAAAA |

| | | |
|---|---|---|
| ORF Start: ATG at 57 | | ORF Stop: TAG at 1965 |
| SEQ ID NO: 214 | 636 aa | MW at 68715.7 kD |

| | |
|---|---|
| NOV36n, | MAGARSRDPWGASGICYLFGSLLVELLFSRAVAFNLDVMGALRKEGEPGSLFGFSVALHR |
| CG56054-15 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGTARVELCAQGSADLAH |
| | LDDGPYEAGGEKEQDPRLIPVPANSYFGFSIPSGKGLVRAEELSFVAGAPRANHKGAVVI |
| | LRKDSASRLVPEVMLSGERLTSGFCYSLAVADLNSDGWPDLIVGAPYFFERQEELGGAVY |
| | VYLNQGGHWAGISPLRLCNSPHSMFGISLAVLGDLNQDGFPDIAVGAPFDGDGKVFIYHG |
| | SSLGVVAKPSQVLEGEAVGIKSFCYSLSGSLDMDGNQYPDLLVGSLADTAVLFRARPILH |
| | VSHEVSIAPRSIDLEQPNCAGGHSVCVDLRVCFSYIAVPSSYSPTVALDYVLDADTDRRL |
| | RGQVPRVTFLSRNLEEPKHQASGTVWLKHQHDRVCGDAMFQLQENVKDKLRAIVVTLSYS |
| | LQTPRLRREGPDAHPILAADGHPELGPDGNPGPGTA |

| | |
|---|---|
| SEQ ID NO: 215 | 1924 bp |

| | |
|---|---|
| NOV36, | TTGGCGCCTGCGAGATTTCCCTTGCATTCGCTCGCAGCTCGCGCAGGGATCGTCCCATGG |
| CG56054-16 | |
| DNA Sequence | CCGGGGCTCGGAGCCGCGACCCTTCCGGGGCCTCCCGGATTTGCTACCTTTTTGGCTCCC |
| | TGCTCGTCGAACTGCTCTTCTCACGGGCTGTCGCCTTCAATCTGGACGTGATGGGTGCCT |
| | TGCGCAAGGAGGGCGAGCCAGGCAGCCTCTTCGGCTTCTCTGTGCCCCTGCACCGGCAGT |
| | TGCAGCCCCGACCCCACAGCTGGCTGCTGGTGGGTCCTCCCCAGGCCCTGGCTCTTCCTG |
| | GGCAGCAGGCCAATCGCACTGCAGGCCTCTTCGCTTCCCCGTTCAGCCTCGAGGAGACTG |
| | ACTGCTACAGAGTGGACATCGACCAGGGACCTGATATGCAAAAGCAAAGCAAGGAGAACC |
| | AGTGGTTGGGAGTCAGTGTTCGGAGCCAGGGGCCTCGGGGCAAGATTGTTACCTCTGCAC |
| | ACCGATATGAGGCAAGGCAGCGAGTGGACCAGATCCTCGAGACGCGCGATATGATTGGTC |
| | GCTGCTTTGTCCTCAGCCAGGACCTGGCCATCCGGGATGAGTTGGATGGTGGGGAATGGA |
| | AGTTCTGTGAGGGACGCCCCCAACGCCATCAACAATTTGCGTTCTGCCAGCAGGGCACAG |
| | CTGCCGCCTTCTCCCCTGATAGCCACTACCTCCTCTTTGGGCCCCCAGGAACCTATAATT |
| | GCAAGGGCACGGCCAGGGTOGAGCTCTGTGCACAGGGCTCAGCGGACCTGGCACACCTGG |
| | ACGACGGTCCCTACGAGGCGGGGGCAGAGAAGGAGCAGGACCCCCOCCTCATCCCGCTCC |
| | CTGCCAACACCTACTTTGGCTTCTCTATTGACTCGGGGAAACGTCTGGTGCCTGCAGAAG |
| | AGCTGAGCTTTGTCGCTCGAGCCCCCCGCGCCAACCACAAGGGTGCTGTGGTCATCCTGC |
| | GCAAGGACAGCGCCAGTCGCCTGGTGCCCGAGGTTATGCTGTCTGCGGAGCGCCTCACCT |
| | CCGGCTTTGGCTACTCACTCGCTGTGGCTGACCTCAACACTGATCGCTGGCCAGACCTGA |
| | TAGTGGGTGCCCCCTACTTCTTTGAGCGCCAAGAAGAGCTGGGGGCTGCTGTGTATGTGT |
| | ACTTGAACCACGCGGCTCACTCGGCTCGGATCTCCCCTCTCCCGCTCTGCGGCTCCCCTG |
| | ACTCCATGTTCGGGATCAGCCTGGCTGTCCTGGGGGACCTCAACCAAGATGGCCTTCCAG |
| | ATATTGCAGTGGGTGCCCCCTTTGATGGTGATGCGAAAGTCTTCATCTACCATCGGAGCA |
| | GCCTCGGGGTTGTCCCCAAGCCTTCACAGGTGCTGGAGGCCGAGGCTGTGGGCATCCCGA |
| | GCTGGCCCCCGATGGGCATCCAGGGCCAGGCACCGCCTAGGTTCCCATGTCCCAGCCTGG |
| | CCTGTGGCTGCCCTCCATCCCTTCCCCAGAGATGGCTCCTTGGGATGAAGACGGTAGAGT |
| | GGGCTGCTGGTGTCGCATCAAGATTTGGCACGATCGGCTTCCTCACGGGCACAGACCTCT |
| | CCCACCCACAAGAACTCCTCCCACCCAACTTCCCCTTAGAGTCCTGTGAGATCAGACTGG |
| | GTAAATCACCGACAGGGCCATCGGGTAGGGTGAGAAGGGCAGGGGTGTCCTCATGCAAAC |
| | GTGGGGAGAACGGATCCTAATCCCTTCCTCTCCCATTCACCCTGTCTAACAGGACCCCAA |
| | GGACCTGCCTCCCCGGAAGTGCCTTAACCTAGACGGTCGCGGAGGAGGTTGTGTCACTGA |
| | CTCAGGCTGCTCCTTCTCTAGTTTCCCCTCTCATCTGACCTTACTTTGCTGCCATCAGTC |
| | TAGTGGTTTCGTGGTTTCGTCTATTTATTAAAAAATATTTGAGAACAAAAAAAAAAAAAA |
| | AAAA |

| | | |
|---|---|---|
| ORF Start: ATG at 57 | | ORF Stop: TGA at 1671 |
| SEQ ID NO: 216 | 538 aa | MW at 57824.0 kD |

| | |
|---|---|
| NOV36, | MAGARSRDPWGASGICYLFGSLLVELLFSRAVAFNLDVMGALRKEGEPCSLFGFSVALHR |
| CG56054-16 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDCGE |
| | WKFCEGRPQGHEQFGFCQQGTAAAFSPDSHYLLFGAPGTYNWKGTARVELCAQGSADLAH |
| | LDDGPYEAGCEKEQDPRLIPVPANSYFGFSIDSGKGLVRAEELSFVAGAPRANHKGAVVI |
| | LRKDSASRLVPEVMLSGERLTSGFGYSLAVADLNSDGWPDLIVGAPYFFERQEELGGAVY |
| | VYLNQGGHWAGISPLRLCGSPDSMFGISLAVLGDLNQDGLPDIAVGAPFDGDGKVFIYHG |
| | SSLGVVAKPSQVLEGEAVGIPSWAPMGIQGQAPPRFPCPSLACGCPPSLPQRWLLGMKRV |
| | EWAAGVASRFGRIGFLRGTDLSHPQELLPPNFPLECCEMRVGKSGTGPWGRVRRAGVS |

| | |
|---|---|
| SEQ ID NO: 217 | 2082 bp |

| | |
|---|---|
| NOV36p, | TTGGGGCGTGCGAGATTTCCCTTGCATTCGCTGGGAGCTCGCGCAGGGATCGTCCCATGG |
| CG56054-17 | |
| DNA Sequence | CCGGGGCTCGGAGCCGCGACCCTTGGCGGGCCTCCGGGATTTGCTACCTTTTTGGCTCCC |
| | TGCTCGTCGAACTGCTCTTCTCACGGGCTGTCGCCTTCAATCTGGACGTGATGGGTGCCT |
| | TGCGCAAGGAGGGCGAGCCAGGCAGCCTCTTCGGCTTCTCTGTGGCCCTGCACCGGCAGT |
| | TGCAGCCCCGACCCCAGAGCTGGCTGCTGGTGGGTGCTCCCCAGGCCCTGGCTCTTCCTG |
| | GGCAGCAGGCGAATCGCACTGGACGCCTCTTCGCTTGCCCGTTGAGCCTGGAGGAGACTG |
| | ACTGCTACAGAGTGGACATCGACCAGGGAGCTGATATGCAAAAGGAAAGCAAGGAGAACC |
| | AGTGGTTGGGAGTCAGTGTTCGGAGCCACGGGCCTGGGGGCAAGATTGTTACCTGTGCAC |
| | ACCGATATGAGGCAAGGCAGCGAGTGGACCAGATCCTGGAGACGCCGGATATGATTGGTC |
| | GCTGCTTTGTGCTCAGCCAGGACCTGGCCATCCGGGATGAGTTCGATGGTCGGGAATGGA |
| | AGTTCTCTGAGGGACGCCCCCAAGGCCATGAACAATTTGGGTTCTGCCAGCAGGGCACAG |
| | CTGCCGCCTTCTCCCCTGATAGCCACTACCTCCTCTTTCGGGCCCCAGGAACCTATAATT |
| | GGAAGGGCACGGCCAGGGTGGAGCTCTGTGCACAGGGCTCAGCGGACCTCGCACACCTGG |
| | ACGACGGTCCCTACCAGGCGGGGCGACAGAACGACCACCACCCCCGCCTCATCCCGGTCC |
| | CTGCCAACAGCTACTTTGGCTTCTCTATTGACTCGCGGAAAGGTCTGGTGCGTGCAGAAG |
| | AGCTGAGCTTTGTCGCTGGAGCCCCCCGCGCCAACCACAAGGGTGCTGTGGTCATCCTGC |
| | GCAAGGACAGCGCCAGTCCCCTGGTGCCCGAGGTTATGCTGTCTGGGCAGCGCCTGACCT |
| | CCGGCTTTGGCTACTCACTGGCTGTGGCTGACCTCAACAGTGATGGCTGGCCAGACCTGA |
| | TAGTGGGTGCCCCCTACTTCTTTGAGCGCCAAGAAGAGCTGGCGGGTGCTGTGTATGTGT |
| | ACTTGAACCAGGCGGGTCACTGGGCTGGGATCTCCCCTCTCCGGCTCTGCGGCTCCCCTG |
| | ACTCCATGTTCGGGATCAGCCTGGCTGTCCTGGGGCACCTCAACCAAGATGCCTGTGGTG |
| | GCAGAAGGAGTGCCCTGGTGGCTCATCCTCCTGGCTGTACTGGCTCGGCTCCTGGTGCTA |
| | GCACTGCTGGTGCTGCTCCTGTGCAACATGGGATTCTTCAAACCGGCGAACCACCCCGAG |
| | GCCACCGTGCCCCAGTACCATGCCCTGAAGATTCCTCGGGAAGACCGACAGCAGTTCAAG |
| | GAGGAGAACACGGGCACCATCCTGAGGAACAACTGCGGCAGCCCCCGGCGCGAGGCCCCG |
| | GATGCACACCCCATCCTGGCTGCTGACGGGCATCCCGAGCTCGGCCCCGATGGGCATCCA |
| | GGGCCAGGCACCGCCTAGGTTCCCATGTCCCAGCCTGCCCTGTGGCTGCCCTCCATCCCT |
| | TCCCCAGAGATCCCTCCTTGGGATGAAGAGGGTAGACTGGGCTGCTGGTCTCGCATCAAG |
| | ATTTGGCAGGATCGCCTTCCTCAGGGCCACAGACCTCTCCCACCCACAAGAACTCCTCCC |
| | ACCCAACTTCCCCTTAGAGTGCTGTCAGATGAGAGTGGGTAAATCACCGACAGGGCCATG |
| | GGGTAGCCTGAGAAGGGCACGGGTGTCCTGATGCAAAGGTGGGGAGAACGGATCCTAATC |
| | CCTTCCTCTCCCATTCACCCTGTGTAACAGGACCCCAAGGACCTGCCTCCCCGGAAGTGC |
| | CTTAACCTAGAGGGTCGGGGAGGAGGTTGTGTCACTGACTCAGGCTGCTCCTTCTCTAGT |
| | TTCCCCTCTCATCTGACCTTAGTTTCCTGCCATCAGTCTAGTGGTTTCGTGCTTTCCTCT |
| | ATTTATTAAAAAATATTTGAGAACAAAAAAAAAAAAAAAAAA |

| | | |
|---|---|---|
| ORF Start: ATG at 57 | | ORF Stop: TGA at 1524 |
| SEQ ID NO: 218 | 489 aa | MW at 51813.5 kD |

| | |
|---|---|
| NOV36p, | MAGARSRDPWGASGICYLFGSLLVELLFSRAVAFNLDVMGALRKEGEPGSLFGFSVALHR |
| CG56054-17 | |
| Protein Sequence | QLQPRPQSWLLVCAPQALALPGQQANRTGGLFACPLSLEETDCYRVDIDQGADMQKESKE |
| | NQWLGVSVRSQGPGGKIVTCAHRYEARQRVDQTLETRDMTGRCFVLSQDLAIRDELDCGE |
| | WKFCEGRPQGHEQFGFCQQGTAAAFSPDSHYLLFGAPGTYNWKGTARVELCAQCSADLAH |
| | LDDGPYEAGGEKEQDPRLIPVPANSYFGFSIDSGKGLVRAEELSFVAGAPRANHKGAVVI |
| | LRKDSASRLVPEVMLSGERLTSGFGYSLAVADLNSDGWPDLIVGAPYFFERQEELGGAVY |
| | VYLNQGGHWAGISPLRLCGSPDSMFGISLAVLGDLNQDGCGGRRSALVGHPPGCTGWAAG |
| | ASTAGAAPVEDGILQTGEAPRGNRAPVPCGEDSSGRPTAVQGCEDGHHPEEQLGQPPAGG |
| | PGCTPHPGC |

| | |
|---|---|
| SEQ ID NO: 219 | 3879 bp |

| | |
|---|---|
| NOV36q, | TTGGGGCGTGCGAGATTTCCCTTGCATTCGCTGGGAGCTCCCGCAGGGATCGTCCCATGG |
| CG56054-18 | |
| DNA Sequence | CCGGGGCTCGGAGCCGCGACCCTTGGGGGGCCTCCGGGATTTGCTACCTTTTTGGCTCCC |
| | TGCTCGTCGAACTGCTCTTCTCACGGGCTGTCGCCTTCAATCTGGACGTGATGGGTGCCT |
| | TGCGCAAGGAGGGCGAGCCAGGCAGCCTCTTCGGCTTCTCTGTGGCCCTGCACCGGCAGT |
| | TGCAGCCCCGACCCCAGAGCTGGCTGCTGGTGGGTCCTCCCCAGGCCCTGGCTCTTCCTG |
| | GGCAGCAGGCGAATCGCACTGGAGGCCTCTTCGCTTGCCCGTTGAGCCTGGAGCACACTG |
| | ACTGCTACAGAGTGGACATCGACCAGGGAGCTGATATGCAAAAGGAAAGCAAGGAGAACC |
| | AGTGGTTCCGAGTCAGTGTTCGGAGCCAGGCGCCTGGGGGCAAGATTGTTACCTGTGCAC |
| | ACCGATATGAGGCAAGGCAGCGAGTGGACCAGATCCTGGAGACGCGGGATATGATTGGTC |
| | GCTGCTTTGTGCTCAGCCAGGACCTGGCCATCCGGGATGAGTTGGATGGTGGGGAATGGA |
| | AGTTCTGTGAGGGACCCCCCCAAGGCCATGAACAATTTGGGTTCTGCCAGCAGGGCACAG |
| | CTGCCGCCTTCTCCCCTGATAGCCACTACCTCCTCTTTGGGGCCCCAGGAACCTATAATT |
| | GGAAGGGCACGGCCAGGGTGGAGCTCTGTGCACAGGGCTCAGCGGACCTGGCACACCTGG |
| | ACGACGGTCCCTACGAGGCGGGGGGAGAGAAGGAGCAGGACCCCCGCCTCATCCCGGTCC |
| | CTGCCAACAGCTACTTTGGCTTCTCTATTGACTCGGGGAAAGGTCTGGTGCGTGCAGAAG |
| | AGCTGAGCTTTGTGGCTGGAGCCCCCCGCGCCAACCACAAGGGTGCTGTGGTTATCCTGC |
| | GCAAGGACAGCGCCAGTCGCCTGGTGCCCGAGGTTATGCTCTCTGGGGAGCGCCTGACCT |
| | CCGGCTTTGGCTACTCACTGGCTGTGGCTGACCTCAACAGTGATGGCTGGCCAGACCTGA |
| | TAGTGGGTGCCCCCTACTTCTTTGAGCGCCAAGAAGAGCTGGGGGCTGCTGTGTATGTGT |
| | ACTTGAACCAGGGCGGTCACTGGGCTGGGATCTCCCCTCTCCGGCTCTGCGGCTCCCCTG |
| | ACTCCATGTTCCCGATCAGCCTGGCTGTCCTGGGGGACCTCAACCAAGATGGCTTTCCAG |
| | ATATTGCAGTGGGTGCCCCCTTTGATGGTGATGGGAAAGTCTTCATCTACCATGGGAGCA |
| | GCCTGGGGGTTGTCGCCAAACCTTCACAGGTGCTGGAGGGCGAGGCTGTGGGCATCAAGA |
| | GCTTCGGCTACTCCCTGTCACGCAGCTTGGATATGCATGGGAACCAATACCCTGACCTGC |
| | TGGTGGGCTCCCTGGCTGACACCGCAGTGCTCTTCAGGGCCAGACCCATCCTCCATGTCT |
| | CCCATGAGCTCTCTATTGCTCCACGAAGCATCGACCTGGAGCAGCCCAACTGTGCTGGCG |
| | GCCACTCGGTCTGTGTCCACCTAACCCTCTGTTTCACCTACATTCCACTCCCCACCAGCT |
| | ATAGCCCTACTGTGGCCCTCGACTATGTCTTAGATGCGGACACAGACCGGACGCTCCGGG |
| | GCCAGGTTCCCCGTGTGACGTTCCTGAGCCGTAACCTGGAAGAACCCAAGCACCAGGCCT |
| | CGGGCACCGTGTGGCTGAAGCACCAGCATGACCGAGTCTGTGGAGACGCCATGTTCCAGC |
| | TCCAGGAAAATCTCAAACACAAGCTTCGGGCCATTGTAGTCACCTTGTCCTACAGTCTCC |
| | AGACCCCTCGGCTCCGGCGACACGCTCCTCGCCAGGCGCTGCCTCCAGTGGCCCCCATCC |
| | TCAATGCCCACCAGCCCAGCACCCAGCGGGCAGAGATCCACTTCCTGAAGCAAGGCTGTG |
| | GTGAAGACAAGATCTGCCACAGCAATCTGCAGCTGGTCCACGCCCCCTTCTGTACCCGGG |
| | TCAGCGACACGGAATTCCAACCTCTGCCCATGGATGTGGATGCAACAACAGCCCTGTTTG |
| | CACTGAGTGGGCAGCCAGTCATTGGCCTGCAGCTGATGGTCACCAACCTCCCATCGGACC |
| | CAGCCCACCCCCAGGCTGATGCGGATGATGCCCATGAAGCCCAGCTCCTGGTCATGCTTC |
| | CTGACTCACTGCACTACTCAGGCGTCCGGGCCCTGGACCCTGCGGAGAAGCCACTCTGCC |
| | TGTCCAATGAGAATGCCTCCCATGTTGAGTGTGAGCTGGGCAACCCCATGAAGAGAGGTG |
| | CCCAGGTCACCTTCTACCTCATCCTTAGCACCTCCGGGATCAGCATTGAGACCACGGAAC |
| | TGGAGGTAGAGCTGCTGTTGGCCACGATCAGTGAGCAGGAGCTGCATCCAGTCTCTGCAC |
| | GAGCCCGTGTCTTCATTGAGCTGCCACTGTCCATTCCAGGAATGGCCATTCCCCAGCAAC |
| | TCTTCTTCTCTGGTGTGGTGACCGCCGAGACACCCATGCAGTCTGAGCGGGATGTGGGCA |
| | GCAAGGTCAAGTATGAGGTCACCGTTTCCAACCAAGCCCAGTCGCTCAGAACCCTGGGCT |
| | CTGCCTTCCTCAACATCATGTGGCCTCATGAGATTGCCAATGGGAAGTGGTTGCTGTACC |
| | CAATGCAGGTTGAGCTGGACGGCGGGCAGCGGCCTCGGCAGAAAGGGCTTTGCTCTCCCA |
| | GGCCCAACATCCTCCACCTCGATGTGGACAGTAGGCATAGGAGGCGGCGGGAGCTGGAGC |
| | CACCTGAGCAGCAGGAGCCTCGTCAGCGGCAGGAGCCCACCATCTCCTGGTCGCCAGTGT |
| | CCTCTCCTGACAAGAAGAAAAACATCACCCTGGACTCCGCCCCGGGCACCGCCAACTGTG |
| | TGGTGTTCAGCTGCCCACTCTACAGCTTTGACCGCGCGGCTGTCCTGCATGTCTGGGGCC |
| | GTCTCTGGAACAGCACCTTTCTCGAGGAGTACTCAGCTGTGAAGTCCCTGGAAGTGATTG |
| | TCCGGGCCAACATCACAGTGAACTCCTCCATAAAGAACTTCATGCTCCGAGATCCCTCCA |
| | CAGTGATCCCACTGATGCTATACTTGGACCCCATGCCTGTGGTGGCAGAACCAGTGCCCT |
| | GGTGGGTCATCCTCCTGCCTGTACTCGCTCGGCTGCTCGTGCTAGCACTGCTGGTGCTGC |
| | TCCTGTCCAAGATCCGATTCTTCAAACGGGCGAAGCACCCCCCCGCGGGACGCCCCGGAT |
| | GCACACCCCATCCTGGCTCCTGACGGGCATCCCGAGCTGGGCCCCGATCCGCATCCAGGG |
| | CCAGGCACCGCCTAGGTTCCCATGTCCCAGCCTGGCCTGTGGCTGCCCTCCATCCCTTCC |
| | CCAGAGATGGCTCCTTGGGATGAAGAGGGTAGAGTGGGCTGCTGGTGTCGCATCAAGATT |
| | TGGCAGGATCGGCTTCCTCAGGGCCACAGACCTCTCCCACCCACAAGAACTCCTCCCACC |
| | CAACTTCCCCTTAGAGTGCTGTGAGATGACACTGGGTAAATCAGGGACAGGGCCATGGGG |
| | TAGGCTGAGAACGGCAGGGGTGTCCTGATGCAAAGGTGGGGACAAGGGATCCTAATCCCT |
| | TCCTCTCCCATTCACCCTGTCTAACAGGACCCCAAGCACCTGCCTCCCCGGAAGTGCCTT |
| | AACCTAGAGGGTCGGGGAGGAGGTTGTGTCACTGACTCAGGCTGCTCCTTCTCTAGTTTC |
| | CCCTCTCATCTGACCTTAGTTTGCTGCCATCAGTCTAGTGGTTTCGTGGTTTCGTCTATT |
| | TATTAAAAATATTTGAGAACAAAAAAAAAAAAAAAAAAA |

| | | |
|---|---|---|
| ORF Start: ATG at 57 | | ORF Stop: TGA at 3321 |
| SEQ ID NO: 220 | 1088 aa | MW at 118618.0 kD |

| | |
|---|---|
| NOV36q | MAGARSRDPWGASGICYLFGSLLVELLPSRAVAFNLDVMGALRKEGEPGSLFGFSVALHR |
| CG56054-18 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGTARVELCAQGSADLAH |
| | LDDGPYEAGGEKEQDPRLIPVPANSYFGFSIDSGKGLVRAEELSFVAGAPRANHKGAVVI |
| | LRKDSASRLVPEVMLSGERLTSGFGYSLAVADLNSDGWPDLIVGAPYFFERQEELGGAVY |
| | VYLNQGGHWAGISPLRLCGSPDSMFGISLAVLGDLNQDGFPDIAVGAPFDGDGKVFIYHG |
| | SSLGVVAKPSQVLEGEAVGIKSFGYSLSGSLDMDGNQYPDLLVGSLADTAVLFRARPILH |
| | VSHEVSIAPRSIDLEQPNCAGGHSVCVDLRVCFSYIAVPSSYSPTVALDYVLDADTDRRL |
| | RGQVPRVTFLSRNLEEPKHQASGTVWLKHQHDRVCGDANFQLQENVKDKLRAIVVTLSYS |
| | LQTPRLRRQAPGQGLPPVAPILNAHQPSTQRAEIHFLKQGCGEDKICQSNLQLVHARFCT |
| | RVSDTEFQPLPMDVDGTTALFALSGQPVIGLELMVTNLPSDPAQPQADCDDAHEAQLLVM |
| | LPDSLHYSGVRALDPAEKPLCLSNENASHVECELGNPMKRGAQVTFYLILSTSGISIETT |
| | ELEVELLLATISEQELHPVSARARVFIELPLSIAGMAIPQQLFFSGVVRGERANQSERDV |
| | GSKVKYEVTVSNQGQSLRTLGSAFLNIMWPHEIANGKWLLYPMQVELEGGQGPGQKGLCS |
| | PRPNILHLDVDSRDRRRRELEPPEOQEPGERQEPSMSWWPVSSAEKKKNITLDCARGTAN |
| | CVVFSCPLYSFDRAAVLHVWGRLWNSTFLEEYSAVKSLEVIVRANITVKSSIKNLMLRDA |
| | STVIPVNVYLDPMAVVAEGVPWWVILLAVLAGLLVLALLVLLLWKMGFFKRAKHPPAGGP |
| | GCTPHPGC |

| | |
|---|---|
| SEQ ID NO: 221 | 2709 bp |

| | |
|---|---|
| NOV36r, | GGGCTTGGGGCGTGCGAGATTTCCCTTGCATTCGCTGGGAGCTCGCGCAGGGATCGTCCC |
| CG56054-19 | |
| DNA Sequence | ATGGCCGGGGCTCGGAGCCGCGACCCTTGGGGGGCCTCCGGGATTTGCTACCTTTTTGGC |
| | TCCCTGCTCGTCGAACTGCTCTTCTCACGGGCTGTCGCCTTCAATCTGGACGTGATGGGT |
| | GCCTTGCGCAAGGAGGCCGAGCCAGGCAGCCTCTTCGGCTTCTCTGTGGCCCTGCACCGG |
| | CAGTTGCAGCCCCGACCCCAGAGCTGGCTGCTGGTGGGTGCTCCCCAGGCCCTGGCTCTT |
| | CCTGGGCAGCAGGCGAATCGCACTGGAGGCCTCTTCGCTTGCCCGTTGAGCCTGGAGGAG |
| | ACTGACTGCTACAGAGTGGACATCGACCAGGGAGCTGATATGCAAAAGGAAAGCAAGGAG |
| | AACCAGTGGTTGGGAGTCAGTGTTCGGAGCCAGGGGCCTGGGGGCAAGATTGTTACCTGT |
| | GCACACCGATATGAGGCAAGGCAGCGAGTGGACCAGATGCTGGAGACGCGGGATATGATT |
| | GGTCGCTGCTTTGTGCTCAGCCAGGACCTGGCCATCCGGGATGAGTTGGATGGTGGGGAA |
| | TGGAAGTTCTGTGAGGGACGCCCCCAAGGCCATGAACAATTTGGGTTCTGCCAGCAGGGC |
| | ACAGCTGCCGCCTTCTCCCCTGATAGCCACTACCTCCTCTTTGGGGCCCCAGGAACCTAT |
| | AATTGGAAGGGGTTGCTTTTTGTGACCAACATTGATAGCTCAGACCCCGACCAGCTGGTG |
| | TATAAAACTTTGGACCCTGCTGACCGGCTCCCAGGACCAGCCGGAGACTTGGCCCTCAAT |
| | AGCTACTTAGGCTTCTCTATTGACTCGGGGAAAGGTCTGGTGCGTGCAGAAGAGCTGAGC |
| | TTTGTGGCTGGAGCCCGCCGCGCCAACCACAAGGGTGCTGTGGTCATCCTGCGCAAGGAC |
| | AGCGCCAGTCGCCTGGTGCCCGAGGTTATGCTGTCTGGGGAGCGCCTGACCTCCGGCTTT |
| | GGCTACTCACTGGCTGTGGCTGACCTCAACAGTGATGGCTGGCCAGACCTGATAGTGGGT |
| | GCCCCCTACTTCTTTGAGCGCCAAGAAGAGCTGGGGGGTGCTGTGTATGTGTACTTGAAC |
| | CAGGGGGGTCACTGGGCTGGGATCTCCCCTCTCCGGCTCTGCGGCTCCCCTGACTCCATG |
| | TTCGGGATCAGCCTGGCTGTCCTGGGGGACCTCAACCAAGATGGCTTTCCAGATATTGCA |
| | GTGGGTGCCCCCTTTGATGGTGATGGGAAAGTCTTCATCTACCATGGGAGCAGCCTGGGG |
| | GTTGTCGCCAAACCTTCACAGGTGCTGGAGGGCGAGGCTGTGGGCATCAAGAGCTTCGGC |
| | TACTGCCTGTCAGGCAGCTTGGATATGGATGGGAACCAATACCCTGACCTGCTGGTGGGC |
| | TCCCTGGCTGACACCGCAGTGCTCTTCAGGGCCAGACCCATCCTCCATGTCTCCCATGAG |
| | GTCTCTATTGCTCCAGGAAGCATCGACCTGGAGCAGCCCAACTGTGCTGGCGGCCACTCG |
| | GTCTGTGTGGACCTAAGGGTCTGTTTCAGCTACATTGCAGTCCCCAGCAGCTATAGCCCT |
| | ACTGTGGCCCTGGACTATGTGTTAGATGCGGACACAGACCGGAGGCTCCGGGGCCAGGTT |
| | CCCCGTGTGACGTTCCTGAGCGGTAACCTGGAAGAACCCAAGCACCAGGCCTCGGGCACC |
| | GTGTGGCTGAAGCACCAGCATGACCGAGTCTGTGGAGACGCCATGTTCCAGCTCCAGGAA |
| | AATGTCAAAGACAAGCTTCGGGCCATTGTAGTGACCTTGTCCTACAGTCTCCAGACCCCT |
| | CGGCTCCGGCGACAGGCTCCTGGCCAGGGGCTGCCTCCAGGGCCTGGGCAGAAAGGGCTT |
| | TGCTCTCCCAGGCCCAACATCCTCCACCTGGATGTGGACAGTAGGGATAGGAGGCGGCGG |
| | GAGCTGGAGCCACCTGAGCAGCAGGAGCCTGGTGAGCGGCAGGAGCCCAGCATGTCCTGG |
| | TGGCCAGTGTCCTCTGCTGAGAAGAAGAAAAACATCACCCTGGACTGCGCCCGGGGCACG |
| | GCCAACTGTGTGGTGTTCAGCTGCCCACTCTACAGCTTTGACCGCGCGGCTGTGCTGCAT |
| | GTCTGGGGCCGTCTCTGGAACAGCACCTTTCTGGAGGAGTACTCAGCTGTGAAGTCCCTG |
| | GAAGTGATTGTCCGGGCCAACATCACAGTGAAGTCCTCCATAAAGAACTTGATGCTCCGA |
| | GATGCCTGCACAGTGATCCCAGTGATGGTATACTTGGACCCCATGGCTGTGGTGGCAGAA |
| | GGAGTGCCCTGGTGGGTCATCCTCCTGGCTGTACTGGCTGGGCTGCTGGTGCTAGCACTG |
| | CTGGTGCTGCTCCTGTGGAAGATGGGATTCTTCAAACGGGCGAAGCACCCCGAGGCCACC |
| | GTGCCCCAGTACCATGCGGTGAAGATTCCTCGGGAAGACCGACAGCAGTTCAAGGAGGAG |
| | AAGACGGGCACCATCCTGAGGAACAACTGGGGCAGCCCCCGGCGGGAGGGCCCGGATGCA |
| | CACCGCATCCTGGCTGCTGACGGGCATCCCGAGCTGGGCCCCGATGGGCATCCAGGGCCA |
| | GGCACCGCCTAGGTTCCCATGTCCCAGCCTGGCCTGTGGCTGCCCTCCATCCCTTCCCCA |
| | GAGATGGCT |

| | | |
|---|---|---|
| ORF Start: ATG at 61 | | ORF Stop: TAG at 2650 |
| SEQ ID NO: 222 | 863 aa | MW at 94348.4 kD |

| | |
|---|---|
| NOV36r, | MAGARSRDPWGASGICYLFGSLLVELLFSRAVAFNLDVMGALRKEGEPGSLFGFSVALHR |
| CG56054-19 | |
| Protein Sequence | QLQPRPQSWLLVGAPQALALPGQQANRTGGLFACPLSLEETDCYRVDIDQGADMQKESKE |
| | NQWLGVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGLLFVTNIDSSDPDQLV |
| | YKTLDPADRLPGPAGDLALNSYLGFSIDSGKGLVRAEELSFVAGAPRANHKGAVVILRKD |
| | SASRLVREVMLSGERLTSGPGYSLAVADLNSDGWPDLIVGAPYFFERQEELGGAVYVYLN |
| | QGGHWAGTSPLRLCGSPDSMFGISLAVLGDLNQDGFPDIAVGAPFDGDGKVFIYHCSSLG |
| | VVAKPSQVLEGEAVGIKSFGYSLSGSLDMDGNQYPDLLVCSLADTAVLFRARPILHVSHE |
| | VSIAPRSTDLEQPNCACGHSVCVDLRVCFSYIAVPSSYSPTVALDYVLDADTDRRLRGQV |
| | PRVTFLSRNLEEPKHQASCTVWLKHQHDRVCGDAMFQLQENVKDKLRAIVVTLSYSLQTP |
| | RLRRQAPGQGLPPGPGQKGLCSPRPNILHLDVDSRDRRRRELEPPEQQEPGERQEPSMSW |
| | WPVSSAEKKKNTTLDCARGTANCVVFSCPLYSFDRAAVLHVWGRLWNSTFLEEYSAVKSL |
| | EVIVRANITVKSSIKNLMLRDASTVIPVMVYLDPMAVVAEGVPWWVILLAVLAGLLVLAL |
| | LVLLLWKMGFFKRAKHPEATVPQYHAVKIPREDRQQFKEEKTGTILRNNWGSPRREGPDA |
| | HPILAADGHPELGPDGHPGPGTA |

| | |
|---|---|
| SEQ ID NO: 223 | 4031 bp |

| | |
|---|---|
| NOV36s, | GGAGCGGCGCGCGGGCGGGAGGGCTGGCGGGGCGAACGTCTGGGAGACGTCTGAAACACC |
| CG56054-02 | |
| DNA Sequence | AACGAGACTTTGGACACCAGAGACGCGCCTCGGCGGACCTGGGGCTTGCGGCGTGCGAGA |
| | TTTCCCTTGCATTCGCTGGGAGCTCGCGCAGGGATCGTCCCATGGCCGGGGCTCGCAGCC |
| | GCGACCCTTGGGGGGCCTCCGGGATTTCCTACCTTTTTGGCTCCCTGCTCGTCGAACTGC |
| | TCTTCTCACGGGCTCTCGCCTTCAATCTGGACGTGATGGGTGCCTTGCGCAAGGAGGGCG |
| | AGCCAGGCAGCCTCTTCGGCTTCTCTGTGGCCCTGCACCGGCAGTTGCAGCCCCGACCCC |
| | AGAGTGGAGGCCTCTTCCCTTGCCCGTTGAGCCTCGAGGAGACTCACTGCTACAGAGTGG |
| | GCACTGGAGGCCTCTTCGCTTGCCCGTTGAGCCTGGAGGAGACTGACTGCTACAGAGTGG |
| | ACATCGACCAGGGAGCTGATATGCAAAAGGAAAGCAAGGAGAACCAGTGGTTGGGAGTCA |
| | GTGTTCGCACCCAGGCGCCTGGGGGCAAGATTGTTACCTGTGCACACCGATATGAGGCAA |
| | GGCAGCGAGTCGACCAGATCCTCGAGACGCGGGATATGATTGGTCGCTCCTTTGTGCTCA |
| | GCCAGGACCTGGCCATCCCGGATGAGTTGGATGGTGGGGAATGGAAGTTCTCTCAGGGAC |
| | GCCCCCAAGGCCATGAACAATTTGGGTTCTCCCAGCAGGCCACAGCTGCCCCCTTCTCCC |
| | CTGATAGCCACTACCTCCTCTTTGGGGCCCCAGGAACCTATAATTGGAAGGGGTTGCTTT |
| | TTGTGACCAACATTGATACCTCACACCCCGACCACCTGGTGTATAAAACTTTGCACCCTG |
| | CTGACCCCCTCCCAGCACCAGCCGGAGACTTGGCCCTCAATAGCTACTTAGGCTTCTCTA |
| | TTGACTCGGGGAAAGGTCTGGTGCGTGCAGAAGAGCTGAGCTTTGTCGCTGGAGCCCCCC |
| | GCGCCAACCACAAGGGTQCTGTGGTTATCCTGCGCAAGGACAGCGCCAGTCGCCTGGTGC |
| | CCGAGGTTATGCTCTCTGGGGACCGCCTCACCTCCCGCTTTGGCTACTCACTGGCTCTGG |
| | CTGACCTCAACAGTGATGGCTGGCCACACCTGATACTGGGTGCCCCCTACTTCTTTGAGC |
| | GCCAAGAACAGCTGGGGGGTGCTGTGTATGTGTACTTGAACCAGGGGGGTCACTGGGCTG |
| | GGATCTCCCCTCTCCGGCTCTGCGGCTCCCCTGACTCCATGTTCGGGATCAGCCTGGCTG |
| | TCCTGGGGGACCTCAACCAAGATGGCTTTCCAGATATTGCAGTGGGTGCCCCCTTTGATG |
| | GTGATGGGAAAGTCTTCATCTACCATGGCAGCAGCCTGGGGGTTGTCGCCAAACCTTCAC |
| | AGGTGCTGGAGGGCGAGGCTGTGGGCATCAAGAGCTTCGGCTACTCCCTGTCAGGCAGCT |
| | TGGATATGGATCGGAACCAATACCCTGACCTGCTGGTCGGCTCCCTGGCTGACACCGCAG |
| | TGCTCTTCAGGGCCAGACCCATCCTCCATGTCTCCCATGAGGTCTCTATTGCTCCACGAA |
| | GCATCGACCTGGAGCAGCCCAACTGTGCTGGCGGCCACTCGGTCTGTGTGGACCTAAGGG |
| | TCTGTTTCAGCTACATTGCAGTCCCCAGCAGCTATAGCCCTACTGTGGCCCTGGACTATG |
| | TGTTAGATGCGGACACAGACCGGAGGCTCCGGGGCCAGGTTCCCCGTGTGACGTTCCTGA |
| | GCCGTAACCTGGAAGAACCCAAGCACCAGGCCTCGGGCACCGTGTGGCTGAAGCACCAGC |
| | ATGACCGAGTCTGTGGAGACGCCATGTTCCAGCTCCAGGAAAATGTCAAAGACAAGCTTC |
| | GGGCCATTGTACTGACCTTGTCCTACAGTCTCCAGACCCCTCGGCTCCGGCGACAGGCTC |
| | CTGGCCAGGGGCTGCCTCCAGTGGCCCCCATCCTCAATGCCCACCAGCCCAGCACCCAGC |
| | GGGCAGAGATCCACTTCCTGAAGCAAGGCTGTGGTCAAGACAAGATCTGCCAGAGCAATC |
| | TGCAGCTGGTCCACGCCCGCTTCTGTACCCGGGTCAGCGACACGGAATTCCAACCTCTGC |
| | CCATGGATGTGGATGGAACAACAGCCCTGTTTGCACTGAGTGGGCAGCCAGTCATTGGCC |
| | TGGAGCTGATGGTCACCAACCTCCCATCGGACCCACCCCAGCCCCAGGCTGATGGGGATG |
| | ATGCCCATGAAGCCCAGCTCCTGGTCATGCTTCCTGACTCACTGCACTACTCAGGGGTCC |
| | GGGCCCTGGACCCTGCGGAGAAGCCACTCTGCCTGTCCAATGAGAATGCCTCCCATGTTG |
| | AGTGTGACCTGGGGAACCCCATGAAGAGAGGTGCCCAGGTCACCTTCTACCTCATCCTTA |
| | GCACCTCCGGGATCAGCATTGAGACCACGGAACTGGAGGTAGAGCTGCTGTTGGCCACGA |
| | TCAGTGAGCACGAGCTGCATCCAGTCTCTGCACGAGCCCGTGTCTTCATTCAGCTGCCAC |
| | TGTCCATTGCAGGAATGGCCATTCCCCAGCAACTCTTCTTCTCTGGTGTGGTGAGGCGCG |
| | AGAGAGCCATGCAGTCTGAGCGCGATGTGGCCAGCAAGGTCAAGTATGAGGTCACGGTTT |
| | CCAACCAAGGCCAGTCGCTCAGAACCCTCGGCTCTGCCTTCCTCAACATCATGTCGCCTC |
| | ATGAGATTGCCAATCGGAAGTGGTTGCTGTACCCAATCCAGGTTGAGCTCGAGGGCGCCC |
| | AGGGGCCTGGGCAGAAAGGGCTTTGCTCTCCCAGGCCCAACATCCTCCACCTGGATGTGG |
| | ACAGTACGGATAGGAGGCGGCGGGAGCTGGAGCCACCTCAGCAGCAGGAGCCTGGTGAGC |
| | GGCAGGAGCCCAGCATGTCCTGGTGGCCAGTGTCCTCTGCTGAGAAGAAGAAAAACATCA |
| | CCCTGGACTGCGCCCGGGGCACGGCCAACTGTGTCGTGTTCAGCTGCCCACTCTACAGCT |
| | TTGACCGCGCGGCTGTGCTGCATGTCTCGGGCCCTCTCTGGAACAGCACCTTTCTGGAGG |
| | AGTACTCACCTGTGAAGTCCCTGGAAGTGATTGTCCGGGCCAACATCACAGTGAAGTCCT |
| | CCATAAAGAACTTGATGCTCCGAGATGCCTCCACAGTGATCCCAGTGATGGTATACTTGG |
| | ACCCCATGCCTGTGGTGGCAGAAGCAGTGCCCTGGTGGGTCATCCTCCTGGCTGTACTGG |
| | CTGGGCTGCTGGTGCTAGCACTGCTGGTGCTGCTCCTGTGCAAGATGGGATTCTTCAAAC |
| | GGGCGAAGCACCCCGAGGCCACCGTGCCCCAGTACQATGCGGTGAAAATTCCTCGCGAAG |
| | ACCGACAGCAGTTCAAGGAGGAGAAGACGGGCACCATCCTCACGAACAACTGGGGCAGCC |
| | CCCATCCTGGCTGGGCCCCGATGGGCATCCAGGGCCAGGCACCGCCTAGGTTCCCATGTC |
| | CCAGCCTGCCCTGTGGCTGCCCTCCATCCCTTCCCCAGAGATGGCTCCTTGGGATGAAGA |
| | GGGTAGAGTGGGCTGCTGGTGTCGCATCAAGATTTGGCAGGATCGGCTTCCTCAGGGCAC |
| | AGACCTCTCCCCCCACAAGAACTCCTCCCACCCAACTTCCCCTTAGAGTGCTGTGAGATG |
| | AGAGTGGGTAAATCAGGGACAGGGCCATGGCGTAGCGTGAGAAGGGCAGGGGTGTCCTGA |
| | TGCAAAGGTGGGGAGAAGGGATCCTAATCCCTTCCTCTCCCATTCACCCTCTGTAACAGG |
| | ACCCCAAGGACCTGCCTCCCCGGAAGTGCCTTAACCTAGAGGGTCGGGGAGGAGGTTGTG |
| | TCACTGACTCAGGCTGCTCCTTCTCTAGTTTCCCCTCTCATCTGACCTTAGTTTGCTGCC |
| | ATCAGTCTAGTGGTTTCGTGGTTTCGTCTATTTATTAAAAAATATTTGAGAACAAAAAAA |
| | AAAAAAAAAAA |

| | | |
|---|---|---|
| ORF Start: ATG at 162 | | ORF Stop: TGA at 3714 |
| SEQ ID NO: 224 | 1184 aa | MW at 129660.8 kD |

| | |
|---|---|
| NOV36s, | MAGARSRDPWGASGICYLFGSLLVELLFSRAVAFNLDVMCALRKEGEPGSLFGPSVALHR |
| CG56054-02 | |
| Protein Sequence | QLQPRPQSWLLVGAPQALALPGQQANRTGGLFACPLSLEETOCYRVDIDQGADMQKESKE |
| | NQWLGVSVRSQGPGGKIVTCAHRYEARQRVDQILETRDMIGRCFVLSQDLAIRDELDGGE |
| | WKFCEGRPQGHEQFGFCQQGTAAAFSPDSHYLLFGAPGTYNWKGLLFVTNIDSSDPDQLV |
| | YKTLDPADRLPGPAGDLALNSYLCFSIDSGKGLVRAEELSFVACAPRANHKGAVVILRKD |
| | SASRLVPEVMLSGERLTSGFGYSLAVADLNSDGWPDLIVCAPYFFERQEELGGAVYVYLN |
| | QGGEWAGISPLRLCCSPDSMFGISLAVLGDLNQDGFPDIAVGAPFDGDGKVFIYHGSSLG |
| | VVAKPSQVLEGEAVGIKSFGYSLSGSLDMDCNQYPDLLVGSLADTAVLFRARPILHVSHE |
| | VSIAPRSIDLEQPNCAGGHSVCVDLRVCFSYIAVPSSYSPTVALDYVLDAETDRRLRGQV |
| | PRVTFLSRNLEEPKHQASGTVWLKHQHDRVCGDAMFQLQENVKDKLRAIVVTLSYSLQTP |
| | RLRRQAPGQGLPPVAPILNAHQPSTQRAEIHFLKQGCGEDKICQSNLQLVHARFCTRVSD |
| | TEFQPLPMDVDGTTALFALSGQPVIGLELMVTNLPSDPAQPQADGDDAHEAQLLVMLPDS |
| | LHYSGVRALDPAEKPLCLSNENASHVECELGNPNKRGAQVTFYLILSTSGISIETTELEV |
| | ELLLATISEQELHPVSARARVPIELPLSIAGMAIPQQLFFSGVVRGERAMQSERDVGSKV |
| | KYEVTVSNQCQSLRTLGSAFLNIMWPHEIANGKWLLYPMQVELEGCQGPGQKGLCSPRPN |
| | ILHLDVDSRDRRRRELEPPEQQEPGERQEPSMSWWPVSSAEKKKNITLDCARGTANCVVF |
| | SCPLYSPDRAAVLHVWGRLWNSTFLEEYSAVKSLEVIVRANITVKSSIKNLMLRDASTVI |
| | PVMVYLDPMAVVAEGVPWWVILLAVLAGLLVLALLVLLLWKMGFFKRAKHPEATVPQYHA |
| | VKIPREDRQQFKEEKTGTILRNNWGSPUPGWAPNGIQGQAPPRFPCPSLACGCPPSLPQR |
| | WLLGMKRVEWAAGVASRFGRIGFLRAQTSPPTRTPPTQLPLRVL |

Sequence comparison of the above protein sequences yields the following sequence relationships shown in Table 36B.

TABLE 36B
Comparison of NOV36a against NOV36b through NOV36s.

| | NOV36a Residues/ | Identities/Similarities |
|---|---|---|
| Protein Sequence | Match Residues | for the Matched Region |
| NOV36b | 1 . . . 607 | 590/607 (97%) |
| | 1 . . . 607 | 591/607 (97%) |
| NOV36c | 1 . . . 439 | 423/439 (96%) |
| | 1 . . . 439 | 424/439 (96%) |
| NOV36d | 1 . . . 142 | 129/142 (90%) |
| | 1 . . . 142 | 129/142 (90%) |
| NOV36e | 1 . . . 64 | 64/64 (100%) |
| | 1 . . . 64 | 64/64 (100%) |
| NOV36f | 1 . . . 113 | 84/113 (74%) |
| | 1 . . . 112 | 86/113 (75%) |
| NOV36g | 1 . . . 395 | 382/395 (96%) |
| | 1 . . . 395 | 382/395 (96%) |
| NOV36h | 1 . . . 276 | 225/286 (78%) |
| | 1 . . . 283 | 233/286 (80%) |
| NOV36i | 1 . . . 1079 | 963/1089 (88%) |
| | 1 . . . 1083 | 969/1089 (88%) |
| NOV36j | 1 . . . 128 | 115/128 (89%) |
| | 1 . . . 128 | 115/128 (89%) |
| NOV36k | 1 . . . 1076 | 997/1076 (92%) |
| | 1 . . . 1076 | 997/1076 (92%) |
| N0V36l | 1 . . . 1079 | 991/1079 (91%) |
| | 1 . . . 1079 | 993/1079 (91%) |
| NOV36m | 1 . . . 1137 | 1011/1147 (88%) |
| | 1 . . . 1134 | 1015/1147 (88%) |
| NOV36n | 1 . . . 607 | 562/617 (91%) |
| | 1 . . . 611 | 567/617 (91%) |
| NOV36o | 1 . . . 439 | 395/449 (87%) |
| | 1 . . . 443 | 400/449 (88%) |
| NOV36p | 1 . . . 395 | 354/405 (87%) |
| | 1 . . . 399 | 358/405 (87%) |
| NOV36q | 1 . . . 1076 | 969/1086 (89%) |
| | 1 . . . 1080 | 973/1086 (89%) |
| NOV36r | 1 . . . 606 | 593/606 (97%) |
| | 1 . . . 606 | 593/606 (97%) |
| NOV36s | 1 . . . 1137 | 1039/1137 (91%) |
| | 1 . . . 1130 | 1039/1137 (91%) |

Further analysis of the NOV36a protein yielded the following properties shown in Table 36C.

TABLE 36C
Protein Sequence Properties NOV36a

| | |
|---|---|
| PSort | 0.4600 probability located in plasma membrane; 0.1363 |
| analysis: | probability located in microbody (peroxisome); 0.1000 |
| | probability located in endoplasmic reticulum (membrane); |
| | 0.1000 probability located in endoplasmic reticulum (lumen) |
| SignalP | Cleavage site between residues 34 and 35 |
| analysis: | |

A search of the NOV36a protein against the Geneseq database, a proprietary database that contains sequences published in patents and patent publication, yielded several homologous proteins shown in Table 36D.

TABLE 36D
Geneseq Results for NOV36a

| | | NOV36a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAB36936 | Integrin alpha chain 7 - | 1 . . . 1137 | 1137/1137 (100%) | 0.0 |
| | Homo sapiens, 1137 aa. | 1 . . . 1137 | 1137/1137 (100%) | |
| | [WO200066628-A1, | | | |
| | 09-NOV-2000] | | | |
| AAU29083 | Human PRO polypeptide | 1 . . . 1137 | 1109/1147 (96%) | 0.0 |
| | sequence#60 - Homo | 1 . . . 1141 | 1113/1147 (96%) | |
| | sapiens, 1141 aa. | | | |
| | [WO200168848-A2, | | | |
| | 20-SEP-2001] | | | |
| AAB44308 | Human PRO768 (UNQ406) | 1 . . . 1137 | 1109/1147 (96%) | 0.0 |
| | protein sequence SEQ ID | 1 . . . 1141 | 1113/1147 (96%) | |
| | NO: 437 - Homo sapiens, | | | |
| | 1141 aa. [WO200053756- | | | |
| | A2, 14-SEP-2000] | | | |
| AAY41752 | Human PRO768 protein | 1 . . . 1137 | 1109/1147 (96%) | 0.0 |
| | sequence - Homo sapiens, | 1 . . . 1141 | 1113/1147 (96%) | |
| | 1141 aa. [WO9946281-A2, | | | |
| | 16-SEP-1999] | | | |
| AAB94058 | Human protein sequence | 159 . . . 1137 | 970/979 (99%) | 0.0 |
| | SEQ ID NO: 14232 - Homo | 1 . . . 973 | 971/979 (99%) | |
| | sapiens, 973 aa. | | | |
| | [EP1074617-A2, | | | |
| | 07-FEB-2001] | | | |

In a BLAST search of public sequence datbases, the NOV36a protein was found to have homology to the proteins shown in the BLASTP data in Table 36E.

TABLE 36E
Public BLASTP Results for NOV36a

| Protein | | Residues/ | Identities/ | |
|---|---|---|---|---|
| Accession | | Match | Similarities for the | Expect |
| Number | Protein/Organism/Length | Residues | Matched Portion | Value |
| JC5950 | integrin alpha-7 chain | 1 . . . 1137 | 1137/1137 (100%) | 0.0 |
| | precursor - human, 1137 aa. | 1 . . . 1137 | 1137/1137 (100%) | |
| Q13683 | Integrin alpha-7 precursor — | 1 . . . 1137 | 1137/1181 (96%) | 0.0 |
| | Homo sapiens (Human) | 1 . . . 1181 | 1137/1181 (96%) | |
| | 1181 aa. | | | |
| I61186 | alpha-7 integrin - mouse, | 14 . . . 1137 | 985/1124 (87%) | 0.0 |
| | 1135 aa. | 14 . . . 1135 | 1046/1124 (92%) | |
| Q61738 | Integrin alpha-7 precursor - | 14 . . . 1137 | 985/1168 (84%) | 0.0 |
| | Mus musculus (Mouse), 1179 | 14 . . . 1179 | 1046/1168 (89%) | |
| | aa. | | | |
| Q63258 | Integrin alpha-7 (H36- | 34 . . . 1137 | 922/1110 (83%) | 0.0 |
| | alpha7) - Rattus norvegicus | 1 . . . 1106 | 981/1110 (88%) | |
| | (Rat), 1106 aa. | | | |

PFam analysis predicts that the NOV36a protein contains the domains shown in Table 36F.

TABLE 36F
Domain Analysis of NOV36a

| | | Identities/ | |
|---|---|---|---|
| | | Similarities | |
| | NOV36a | for the Matched | |
| Pfam Domain | Match Region | Region | Expect Value |
| FG-GAP | 49 . . . 114 | 20/67 (30%) | 6.1e−11 |
| | | 48/67 (72%) | |
| FG-GAP | 260 . . . 317 | 20/66 (30%) | 5.4e−06 |
| | | 42/66 (64%) | |
| FG-GAP | 318 . . . 377 | 26/65 (40%) | 1.3e−14 |
| | | 49/65 (75%) | |
| FG-GAP | 378 . . . 435 | 30/67 (45%) | 2.2e−18 |
| | | 51/67 (76%) | |
| FG-GAP | 436 . . . 489 | 20/66 (30%) | 6.1e−08 |
| | | 42/66 (64%) | |
| integrin_A | 1061 . . . 1075 | 7/15 (47%) | 0.0074 |
| | | 14/15 (93%) | |

### Example 37

The NOV37 clone was analyzed, and the nucleotide and encoded polypeptide sequences are shown in Table 37A.

TABLE 37A
NOV37 Sequence Analysis

| | |
|---|---|
| SEQ ID NO: 225 | 4096 bp |

| | |
|---|---|
| NOV37a, | ATCTGTTTTATTTATTTCTGTTAATTTCCAATAGTATAATTTGACATGCATTTCTGTTTT |
| CG88634-01 | |
| DNA Sequence | GTCTTTTCAGGTGCCATTTGGATTGTACTTTAGTGGCACGATGTACTCTGAGTGGAGGTC |
| | ACTGCATTTGGTCATTCAGAATGATCAAGGCCATACCAGTGTGCTGCACAGCTATCCAGA |
| | GAGCCTTGGACGAGAGGTCGCAAATGCTGTAGTCCGTCCTCTTGGGCAGGTGTTAGGTAC |
| | CCCTTCAGTGGCTGGTAGTGAGAATTTGTTAAAAACTGACAAAGAAGTAAAATGGACCAT |
| | GGAAGTAATTTGCTATGGACTGACCCTTCCATTGGATGCAGAGACTGTAAAATATTGCGT |
| | TCATCTATATACAGACTGGATTATGGCTTTAGTGTTGCCAAAAGATTCTATTCCATTGCC |
| | AGTTATTAAAGAGCCTAATCAATATGTTCAAACTATACTAAAACACCTACAGAATCTTTT |
| | TGTACCAAGACAGGAACAGGGTTCCAGTCAGATTCGACTATGCTTACAGGTCCTGAGAGC |
| | CATTCAGAAACTGGCCCGTGAGTCATCTCTCATGGCCCGAGAAACTTGGGAAGTCTTACT |
| | GTTGTTTCTTCTGCACATTAACGACATACTTCTGGCCCCACCAACTGTTCAAGGTTTGAT |
| | TGCTGAGAATCTAGCAGAGAAGTTGATTGGTGTTCTCTTTGAGGTGTGGTTACTAGCTTC |
| | TACTCCGTGCTTCCCAACACCTCCTTATTGGAAAACAGCCAAGGACATGGTCGCTAACTG |
| | GAGGCATCACCCAGCAGTGGTCGAGCAGTGGAGCAAGGTCATTTCTGCACTCACTTCCAG |
| | GTTACTACGCTTTACATATGGTCCTTCATTTCCTGCATTTAAAGTTCCCGATGAAGATGC |
| | CAGTCTGATCCCTCCAGAAATGGATAATCAGTGTGTTGCACAGACATGGTTTCGCTTTTT |
| | ACACATGTTAACTAATCCTGTGGATTTGAGTAACCCACCTATTATAAGCTCTACTCCCAA |
| | ATTTCAGGAACAGTTCTTGAATGTCAGCGCAATGCCCCAAGAATTGAATCAGTATCCCTG |
| | CCTTAAACATCTGCCTCAAATATTTTTTCGTGCCATGCGTGGAATCAGCTGTCTGGTGGA |
| | TGCATTCTTAGGTATTTCTAGACCCCGATCAGACAGTGCTCCCCCAACACCCGTGAATAG |
| | ATTAAGTATGCCTCAAAGTGCTGCTGTCAGTACCACCCCCCCACATAACCGGAGGCACCC |
| | GGCTCTTACTGTGAATAAGGCCACCATGAAGACAAGCACAGTTAGTACTGCTCATCCCTC |
| | TAAAGTTCAGCACCAGACGTCCTCCACCTCTCCTCTGTCAAGTCCAAATCAGACTAGTTC |
| | AGAACCCCGGCCACTCCCTGCCCCTCGGAGACCAAAGGTTAACAGCATCTTGAATCTCTT |
| | GTCCACAGACACCATGGTTTCCAATCCTATGTTTGATGCAAGTGAATTTCCTGATAACTA |
| | TGAACCAGGAAGAGCTCAGCCTTGTGGGACACTGTGTAGGATTTTTTGTAGCAAGAAGAC |
| | TGGAGAAGAGATTCTGCCAGCTTATTTATCCACATTTTACATGCTTTTAATTCAAGGTTT |
| | GCAGATAAATCATTATGTGTGCCATCCTCTCTTGGCCAGCGTTATTCTAAACTCTCCTCC |
| | TTTGTTCTGCTGTGACTTGAAAGCGATTGATGTTGTGGTTCCTTACTTTATTTCACCTCT |
| | TGAAACCATTTTGCCTGACAGGAGAGAACTCTCAAAATTCAAAAGCTATGTAAATCCAAC |
| | AGAATTGCGAAGATCCTCCATTAATATCCTCCTTTCTTTGTTGCCCCTCCCTCATCATTT |
| | TGGCACAGTCAAATCTCAGTCTTATGATAAACCAATAACTTTTCTGTCCCTGAAGTTGAC |
| | ACTTGTGAATATATTAATAGGTCCCTTGCAAACTGAAACGGACCCCAACAACACCCAAAT |
| | ACTCAACTCCCAGTGCCGCCAAGACATGAGCATATCACTGGCAGCTCTAGAGCTCCTCTC |
| | TGGCCTTGCAAAGGTCAGGAACACACACTCAGGAGACCGGAAGCGAGCCATCACTTCTGT |
| | GTGCACCTACATTGTTTATCAGTGTAGTCGGCCAGCTCCTTTACACTCCAGGGATCTGCA |
| | CTCCATGATAGTGGCAGCTTTTCAGTGTCTCTGTGTCTGGCTGACAGAGCACCCTGATAT |
| | GCTTGATGAAAAGGACTGCCTTAAGGAAGTACTGGAGATTGTGGAACTGGGTATCTCAGG |
| | AAGTAAGTCCAAGAACAATGAGCAAGAGGTCAAGTACAAAGGAGATAAGGAGCCAAACCC |
| | TGCATCTATGAGGGTAAACGATGCTGCTGAAGCCACCCTAACATCCATTCTCCATAGCAT |
| | TGGCGCATTTCCTTCACCTAGTGGTCCTGCCTCTCCTTGTAGTCTTGTGAATGACACCAC |
| | TTTGATTAAATACTCCAGGCTGCCAACCATAAACAAGCATAGTTTCCGGTACTTTGTCTT |
| | GGATAACAGTGTCATCCTGGCAATGCTGGAACAACCTCTTGGAAATGAGCAGAATGATTT |
| | TTTCCCCTCTGTCACTGTGCTGGTCCGGGGAATGTCTGGAAGACTTGCTTGGGCACAACA |
| | GCTTTGTCTTTTACCCAGAGGAGCAAAAGCAAATCAGAAGCTTTTTGTACCTGAACCTCG |
| | CCCAGTTCCTAAAAATGACGTTGGATTTAAATATTCTGTGAAACATCGGCCATTTCCTGA |
| | AGAGGTCGACAAGATTCCTTTTGTGAAAGCAGATCTCAGCATTCCAGATTTCCATGAAAT |
| | AGTCACTGAAGAATTAGAAGAGAGACACGAAAAATTAAGGAGTGGCATGCCCCAGCAGAT |
| | TGCTTATGAAATACACCTTGAGGACAAGAGTGAGGAGGAATTGCAGAAGAGAAGTTTTCC |
| | TGACCCAGTTACGGATTGCAAGCCCCCGCCTCCTGCCCAGCAATTCCAAACAGCCCGCCT |
| | TTTTCTCTCACACTTTGGATTTTTGTCCTTAGAAGCACTGAAGGAACCTGCAAATACTCG |
| | TCTACCTCCTCACCTTATTGCACTTGATTCCACGATACCTGGATTTTTTGATGACATTGG |
| | GTATCTGGATCTCTTGCCATGTCGTCCTTTTCACACAGTTTTTATTTTCTATATGAAGCC |
| | AGGTCAGAAAACGAACCAACAGATTTTAAAGAATGTGGAGTCTTCCAGAACTGTTCAGCC |
| | ACATTTCCTAGAATTTTTGCTTTCCCTTGGCTGGTCAGTAGATGTGGGCAGACACCCTGG |
| | TTGGACTGGGCATGTTTCTACCAGTTGGTCTATTAATTGTTGTGATGATGGTGAAGGATC |
| | TCAACAAGAAGTGATTTCCTCTGAAGATATTGGAGCTAGCATTTTCAATGGACAGAAGAA |
| | GGTGCTCTATTATGCTGATGCCCTTACAGAAATTGCTTTTGTGGTTCCTTCTCCTGTGGA |
| | GTCCTTAACTGATTCATTGGAAAGTAACATCTCGCACCAAGATAGTGATTCAAATATGGA |
| | TCTTATGCCAGGAATTCTGAAACAGCCATCCCTGACACTTGAGCTTTTCCCCAATCATAC |
| | AGACAATCTTAATTCCTCACAGAGGCTCAGTCCCAGTTCCAGAATGAGGAAGCTCCCTCA |
| | GGGTCGCCCTGTTCCTCCCCTTGGACCTGAGACAAGAGTTTCTGTAGTCTCGCTGGAACG |
| | CTATGATGATATAGAAAACTTTCCCCTCTCAGAGCTCATGACAGAGATCAGTACTGGTGT |
| | GGAAACTACTGCAAATAGTAGCACTTCACTGAGATCTACAACTCTTGAAAAAGAAGTTCC |
| | TGTCATCTTCATCCACCCTTTAAACACTCCATTATTCCGGATAAAAATTCAAGGAGCCAC |
| | TGGAAAATTTAATATGGTCATCCCTCTTGTGGATGGGATGATTGTCAGCAGGCGAGCTCT |
| | TGGCTTTCTGGTGAGG |

| | | |
|---|---|---|
| OFR Start: ATG at 101 | | ORF Stop: end of sequence |
| SEQ ID NO: 226 | 1332 aa | MW at 149066.8 kD |

| | |
|---|---|
| NOV37a, | MYSEWRSLHLVIQNDQGHTSVLHSYPESVGREVANAVVRPLGQVLGTPSVAGSENLLKTD |
| CG88634-01 | |
| Protein Sequence | KEVKWTMEVICYGLTLPLDCETVKYCVDVYTDWIMALVLPKDSIPLPVIKEPNQYVQTIL |
| | KHLQNLFVPRQEQGSSQIRLCLQVLRAIQKLARESSLMARETWEVLLLFLLQINDILLAP |
| | PTVQGLIAENLAEKLIGVLFEVWLLACTRCFPTPPYWKTAKEMVANWRHHPAVVEQWSKV |
| | TCALTSRLLRFTYGPSFPAPKVPDEDASLIPPEMDNECVAQTWFRFLHMLSNPVDLSNPA |
| | IISSTPKFQEQFLNVSGMPQELNQYPCLKHLPQIFFRAMRGISCLVDAFLGISRPRSDSA |
| | PPTPVNRLSMPQSAAVSTTPPHNRRHRAVTVNKATMKTSTVSTAHASKVQHQTSSTSPLS |
| | SPNOTSSEPRPLPAPRRPKVNSILNLPGSWLFDAAFVMEFRRKGSQMSTDTMVSNPMPDA |
| | SEPPDNYEAGRAEACGTLCRIFCSKKTGEEILPAYLSRFYMLLIQGLQINDYVCHPVLAS |
| | VILNSPPLFCCDLKGIDVVVPYFISALETILPDRRELSKFKSYVNPTELRRSSINILLSL |
| | LPLPHHFGTVKSESYDKPITFLSLKLRLVNILIGALQTETDPNNTQMILGDSAAGLLIRS |
| | LHSRDLHSMIVAAFQCLCVWLTEHPDMLDEKDCLKEVLEIVELGISGSKSKNNEQEVKYK |
| | IHLVTORLNSQWRQDMSISLAALELLSGLAKVRKTDSGDRKRATSSVCTYTVYQCSRPAP |
| | GDKEPNPASMRVKDAAEATLTSILHSIGAFPSPSGPASPCSLVNETTLIKYSRLPTINKH |
| | LFVPEPRPVPKNDVGFKYSVKHRPFPEEVDKIPFVKADLSIPDLHEIVTEELEERHEKLR |
| | SCMAQQIAYEIHLEQQSEEELQKRSFPDPVTDCKPPPPAQEFQTARLFLSHFGFLSLEAL |
| | KEPANSRLPPHLIALDSTIPGFFDDIGYLDLLPCRPFDTVFTFYMKPGQKTNQEILKNVE |
| | SSRTVQPHFLEFLLSLGWSVDVGRHPGWTGHVSTSWSINCCDDGEGSQQEVISSFDIGAS |
| | TFNGQKKVLYYADALTEIAFVVPSPVESLTDSLESNISDQDSDSNMDLMPGILKQPSLTL |
| | ELFPNHTDNLNSSQRLSPSSRMRKLPQGRPVPPLGPETRVSVVWVERYDDIENFPLSELM |
| | TEISTGVETTANSSTSLRSTTLEKEVPVIFIHFLNTGLFRTKTQGATGKFNMVIPLVDGM |
| | IVSRRALGPLVR |

Two polymorphic variants of NOV37a have been identified and are shown in Table 41O. Further analysis of the NOV37a protein yielded the following properties shown in Table 37B.

TABLE 37B
Protein Sequence Properties NOV37a

| | |
|---|---|
| PSort | 0.7900 probability located in plasma membrane; 0.3500 |
| analysis: | probability located in nucleus; 0.3000 probability located in |
| | microbody (peroxisome); 0.3000 probability located in |
| | Golgi body |
| SignalP | No Known Signal Sequence Predicted |
| analysis: | |

A search of the NOV37a protein against the Geneseq database, a proprietary database that contains sequences published in patents and patent publication, yielded several homologous proteins shown in Table 37C.

TABLE 37C
Geneseq Results for NOV37a

| | | Residues/ | Similarities for | |
|---|---|---|---|---|
| Geneseq | Protein/Organism/Length | Match | the Matched | Expect |
| Identifier | [Patent #, Date] | Residues | Region | Value |
| AAM39605 | Human polypeptide SEQ ID | 878 . . . 1332 | 455/456 (99%) | 0.0 |
| | NO 2750 - Homo sapiens, | 1 . . . 456 | 455/456 (99%) | |
| | 515 aa. [WO200153312-A1, | | | |
| | 26-JUL-2001] | | | |
| AAM41391 | Human polypeptide SEQ ID | 1072 . . . 1332 | 261/262 (99%) | e−147 |
| | NO 6322 - Homo sapiens, | 1 . . . 262 | 261/262 (99%) | |
| | 321 aa. [WO200153312-A1, | | | |
| | 26-JUL-2001] | | | |
| ABB58732 | Drosophila melanogaster | 1 . . . 658 | 309/705 (43%) | e−l41 |
| | polypeptide SEQ ID NO | 1 . . . 660 | 412/705 (57%) | |
| | 2988 - Drosophila | | | |
| | melanogaster, 1523 aa. | | | |
| | [WO200171042-A2, | | | |
| | 27-SEP-2001] | | | |
| AAB43113 | Human ORFX ORF2877 | 1171 . . . 1332 | 162/162 (100%) | 3e−87 |
| | polypeptide sequence SEQ | 1 . . . 162 | 162/162 (100%) | |
| | ID NO: 5754 - Homo | | | |
| | sapiens, 221 aa. | | | |
| | [WO200058473-A2, | | | |
| | 05-OCT-2000] | | | |
| AAB41768 | Human ORFX ORF1532 | 683 . . . 801 | 115/120 (95%) | 6e−59 |
| | polypeptide sequence SEQ | 2 . . . 121 | 116/120 (95%) | |
| | ID NO: 3064 - Homo | | | |
| | sapiens, 128 aa. | | | |
| | [WO200058473-A2, | | | |
| | 05-OCT-2000] | | | |

In a BLAST search of public sequence datbases, the NOV37a protein was found to have homology to the proteins shown in the BLASTP data in Table 37D.

TABLE 37D
Public BLASTP Results for NOV37a

| | | NOV37a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9H3X8 | DJ927M24.2 (KIAA1219) - | 1 . . . 1169 | 1143/1208 (94%) | 0.0 |
| | Homo sapiens (Human), | 1 . . . 1188 | 1144/1208 (94%) | |
| | 1188 aa (fragment). | | | |
| BAA86533 | KIAA1219 protein - Homo | 651 . . . 1332 | 674/683 (98%) | 0.0 |
| | sapiens (Human), 1112 aa | 371 . . . 1053 | 677/683 (98%) | |
| | (fragment). | | | |
| CAD39096 | Hypothetical protein - Homo | 651 . . . 1332 | 674/684 (98%) | 0.0 |
| | sapiens (Human), 1333 aa | 591 . . . 1274 | 677/684 (98%) | |
| | (fragment). | | | |
| Q9ULK1 | KIAA1219 protein - Homo | 860 . . . 1332 | 473/473 (100%) | 0.0 |
| | sapiens (Human), 532 aa | 1 . . . 473 | 473/473 (100%) | |
| | (fragment). | | | |
| Q8WWC0 | Hypothetical 47.6 kDa | 970 . . . 1332 | 363/363 (100%) | 0.0 |
| | protein - Homo sapiens | 2 . . . 364 | 363/363 (100%) | |
| | (Human), 423 aa (fragment). | | | |

PFam analysis predicts that the NOV37a protein contains the domains shown in Table 37E.

TABLE 37E
Domain Analysis of NOV37a

| | | | |
|---|---|---|---|
| Pfam | NOV37a Match Region | Identities/ | Expect Value |
| Domain | | Similarities | |
| | | for the Matched | |
| | | Region | |

### Example 38

The NOV38 clone was analyzed, and the nucleotide and encoded polypeptide sequences are shown in Table 38A.

TABLE 38A
NOV38 Sequence Analysis

| | |
|---|---|
| SEQ ID NO: 227 | 3116 bp |

| | |
|---|---|
| NOV38a, | ATGCCCGCCGCCCGGCCCCCCGCCGCCCGCCTGCGGGGCATCAGCCTGTTCCTGCCCCTG |
| CG97012-01 | |
| DNA Sequence | CTGCTGGCCAGCCCCGCCGCCGCCCTGGAGCGGGACGCCCTCCCCGAGCGCGACGCCAGC |
| | CCCCTGGGCCCCTACCTGCTGCCCAGCGGCGCCCCCGAGCGGGGCAGCCCCGGCAAGGAG |
| | CACCCCGAGGAGCGGGTGGTGACCGCCCCCCCCAGCAGCAGCCAGAGCGCCCAGGTGCTC |
| | CGCGAGCTGGTGCTCGACGGCACCGCCCCCAGCCCCCACCACCACATCCCCGCCCTGAGC |
| | CCCCTGCTGCCCGAGGAGGCCCGGCCCAAGCACGCCCTGCCCCCCAAGAAGAAGCTCCCC |
| | AGCCTGAAGCAGGTGAACAGCGCCCGGAAGCAGCTGCGGCCCAAGGCCACCAGCGCCGCC |
| | ACCGTGCAGCGGGCCGGCAGCCAGCCCGCCAGCCAGGGCCTGGACCTGCTGAGCAGCAGC |
| | ACCGAGAAGCCCGGCCCCCCCGCCGACCCCGACCCCATCGTGGCCAGCGAGGAGGCCAGC |
| | GAGGTGCCCCTGTGCCTGGACCGGAAGCAGAGCGCCGTGCCCACCACCCCCGCACCCCTG |
| | CAAATCTCCCCCTTCACTTCCCAGCCCTATGTGGCCCACACACTCCCCCAGAGGCCAGAA |
| | CCCGGCGAGCCTGGGCCTGACATGGCCCAGGAGCCCCCCCAGGAGGACACCAGCCCCATG |
| | GCCCTGATGGACAAAGGTGAGAATGAGCTGACTGGGTCAGCCTCAGACGAGAGCCAGGAG |
| | ACCACTACCTCCACCATTATCACCACCACGGTCATCACCACCGAGCAGGCACCAGCTCTC |
| | TGCAGTGTGAGCTTCTCCAATCCTGAGGGGTACATTGACTCCAGCCACTACCCACTGCTG |
| | CCCCTCAACAACTTTCTCGAGTCCACATACAACGTGACAGTCTACACTGGCTATGGGGTG |
| | GAGCTCCACGTGAAGAGTGTGAACCTGTCCGATGGGGAACTGCTCTCCATCCGCGGGGTG |
| | GACGGCCCTACCCTGACCGTCCTGGCCAACCAGACACTCCTGGTGGAGGGGCAGGTAATC |
| | CGAAGCCCCACCAACACCATCTCCGTCTACTTCCGGACCTTCCAGGACGACGGCCTTGGG |
| | ACCTTCCAGCTTCACTACCAGGCCTTCATGCTGAGCTGCAACTTTCCCCOCCGGCCTGAC |
| | TCTGGGGATGTCACGGTGATGGACCTGCACTCAGGTGGGGTGGCCCACTTTCACTGCCAC |
| | CTGGGCTATGAGCTCCAGGGCGCTAACATGCTGACATGCATCAATGCCTCCAAGCCGCAC |
| | TGGAGCAGCCAGGAGCCCATCTGCTCACCTCCTTGTGGAGGCGCAGTGCACAATGCCACC |
| | ATCGCCCGCGTCCTCTCCCCAAGTTACCCTGAAAACACCAATGGGAGCCAATTCTGCATC |
| | TGGACGATTGAAGCTCCAGAGGGCCAGAAGCTGCACCTGCACTTTGAGAGGCTGTTGCTG |
| | CATGACAAGGACAGGATGACGGTTCACAGCCGGCAGACCAACAAGTCAGCTCTTCTCTAC |
| | GACTCCCTTCAAACCGAGAGTGTCCCTTTTGAGGGCCTGCTGAGCCAAGGCAACACCATC |
| | CGCATCGAGTTCACGTCCCACCAGGCCCGGGCGGCCTCCACCTTCAACATCCGATTTGAA |
| | GCGTTTGAGAAAGGCCACTCCTATGACCCCTACATCCAGAATGGGAACTTCACTACATCC |
| | GACCCGACCTATAACATTGGGACTATAGTGGACTTCACCTGCGACCCCGGCCACTCCCTG |
| | GAGCAGGGCCCGGCCATCATCGAATGCATCAATGTGCGGGACCCATACTGGAATGACACA |
| | GAGCCCCTGTGCAGAGCCATCTGTGCTGGGGAGCTCTCTGCTGTGGCTGGGCTGGTATTG |
| | TCCCCAAACTGGCCCGAGCCCTACGTGGAAGGTGAAGATTGTATCTGGAAGATCCACGTG |
| | GGAGAAGAGAAACGGATCTTCTTAGATATCCAGTTCCTGAATCTGACCAACAGTGACATC |
| | TTGACCATCTACGATGGCCACGAGGTCATGCCCCACATCTTCGGCCAGTACCTTGGGAAC |
| | AGTGGCCCCCAGAAACTGTACTCCTCCACGCCAGACTTAACCATCCAGTTCCATTCGGAC |
| | CCTCCTGGCCTCATCTTTGGAAAGCCCCAGGCATTTATCATGAACTACATAGAGGTATCA |
| | AGCAATGACTCCTGCTCGGATTTACCCGAGATCCAGAATGGCTGGAAAACCACTTCTCAC |
| | ACGGAGTTGGTGCGGGGAGCCAGAATCACCTACCACTGTGACCCCGGCTATGACATCGTG |
| | GGGAGTGACACCCTCACCTGCCAGTCGGACCTCAGCTCGAGCAGCGACCCCCCATTTTGT |
| | GAGAAAATTATGTACTGCACCGACCCCGGAGAGGTCGATCACTCGACCCCCTTAATTTCG |
| | GATCCTGTGCTCCTGGTOGGGACCACCATCCAATACACCTGCAACCCCGCTTTTGTGCTT |
| | GAAGGGAGTTCTCTTCTGACCTGCTACAGCCGTGAAACAGGGACTCCCATCTGGACGTCT |
| | CGCCTGCCCCACTGCGTTTCGGAGGAGTCCCTGGCATGTGACAACCCAGGGCTGCCTGAA |
| | AATGGATACCAAATCCTGTACAAGCGACTCTACCTCCCAGGAGAGTCCCTCACCTTCATG |
| | TGCTACGAAGGCTTTGACCTCATGGGTGAAGTGACCATCCGCTGCATCCTGGCACAGCCA |
| | TCCCACTGGAACGGGCCCCTGCCCCTGTGTAAAGTACCAGAAGCGGCAGCAGAGACGTCG |
| | CTGGAAGGCGGGAACATGGCCCTGGCTATCTTCATCCCGGTCCTCATCATCTCCTTACTG |
| | CTGGCAGGAGCCTACATTTACATCACAAGATGTCGCTACTATTCCAACCTCCCCCTCCCT |
| | CTGATGTACTCCCACCCCTACAGCCAGATCACCCTCGAAACCGAGTTTGACAACCCCATT |
| | TACGAGACAGGCGAAACCAGAGAGTATGAGGTTTCTATCTAAACACAGCTACACTTGAGA |
| | AGGGGACTTGTGAACTCAACCACAATCTCCTCCACACATTCATCCACAGACCATGT |

| | | |
|---|---|---|
| ORF Start: ATG at 1 | | ORF Stop: TAA at 3040 |
| SEQ ID NO: 228 | 1013 aa | MW at 110509.9 kD |

| | |
|---|---|
| NOV38a, | MPAARPPAAGLRGISLFLALLLGSPAAALERDALPEGDASPLGPYLLPSGAPERGSPGKE |
| CG97012-01 | |
| Protein Sequence | HPEERVVTAPPSSSQSAEVLGELVLDGTAPSAHHDIPALSPLLPEEARPKHALPPKKKLP |
| | SLKQVNSARKQLRPKATSAATVQRAGSQPASQGLDLLSSSTEKPGPPGDPDPIVASEEAS |
| | EVPLWLDRKESAVPTTPAPLQTSPFTSQPYVAHTLPQRPEPGEPGPDMAQEAPQEDTSPM |
| | ALMDKGENELTGSASEESQETTTSTIITTTVITTEQAPALCSVSFSNPEGYIDSSDYPLL |
| | PLNNFLECTYNVTVYTGYGVELQVKSVNLSDGELLSIRCVDGPTLTVLANQTLLVEGQVI |
| | RSPTNTISVYFRTFQDDCLGTFQLHYQAFMLSCNFPRRPDSGDVTVMDLHSGGVAHFHCH |
| | LGYELQGAKMLTCINASKPHWSSQEPICSAPCGGAVHNATIGRVLSPSYPENTNGSQFCI |
| | WTIEAPEGQKLHLHFERLLLHDKDRMTVHSGQTNKSALLYDSLQTESVPFEGLLSEGNTI |
| | RIEFTSDQARAASTFNIRFEAFEKGHCYEPYIQNGNFTTSDPTYNIGTIVEFTCDPGHSL |
| | EQGPAIIECINVRDPYWNDTEPLCRAMCGGELSAVAGVVLSPNWPEPYVEGEDCIWKLHV |
| | GEEKRIFLDIQFLNLSNSDILTIYDGDEVMPHTLGQYLGNSGPQKLYSSTPDLTIQFHSD |
| | PAGLIFCKGQGFIMNYIEVSRNDSCSDLPEIQNCWKTTSHTELVRGARITYQCDPGYDIV |
| | GSDTLTCQWDLSWSSDPPFCEKINYCTDPGEVDHSTRLISDPVLLVCTTIQYTCNPGFVL |
| | EGSSLLTCYSRETGTPIWTSRLPHCVSEESLACDNPGLPENGYQILYKRLYLPGESLTFM |
| | CYEGFELMGEVTIRCILGQPSHWNGPLPVCKVAEAAAETSLEGGNMALAIFIPVLIISLL |
| | LGGAYIYITRCRYYSNLRLPLMYSHPYSQITVETEFDNPIYETGETREYEVSI |

| | |
|---|---|
| SEQ ID NO: 229 | 2420 bp |

| | |
|---|---|
| NOV38b, | CCTGGGCCTCACATGGCCCAGGACGCCCCCCAGGAGGACACCAGCCCCATGGCCCTGATG |
| CG97012-02 | |
| DNA Sequence | GACAAAGGTGAGAATGAGCTGACTGGCTCAGCCTCAGACGAGAGCCAGGAGACCACTACC |
| | TCCACCATTATCACCACCACGGTCATCACCACCGAGCAGGCACCAGCTCTCTGCAGTGTG |
| | AGCTTCTCCAATCCTGAGGGGTACATTGACTCCAGCGACTACCCACTGCTGCCCCTCAAC |
| | AACTTTCTGGAGTGCACATACAACGTGACAGTCTACACTGGCTATGGGGTGGAGCTCCAG |
| | GTGAAGAGTGTGAACCTGTCCGATGGGGAACTGCTCTCCATCCGCGGGGTGGACGGCCCT |
| | ACCCTGACCGTCCTGGCCAACCAGACACTCCTGGTGGAGGGGCAGGTAATCCGAAGCCCC |
| | ACCAACACCATCTCCGTCTACTTCCGGACCTTCCAGGACGACGGCCTTGGGACCTTCCAG |
| | CTTCACTACCAGGCCTTCATGCTGAGCTGCAACTTTCCCCCCCGGCCTGACTCTGGGGAT |
| | GTCACGGTGATGGACCTGCACTCACGTGGGGTGGCCCACTTTCACTGCCACCTGGGCTAT |
| | GAGCTCCAGGGCGCTAAGATGCTGACATGCATCAATGCCTCCAACCCGCACTGGAGCAGC |
| | CAGGAGCCCATCTGCTCAGCTCCTTGTGGAGGGGCAGTGCACAATGCCACCATCGGCCGC |
| | GTCCTCTCCCCAAGTTACCCTCAAAACACAAATGGGAGCCAATTCTGCATCTGGACGATT |
| | GAAGCTCCAGAGGGCCAGAAGCTGCACCTGCACTTTGAGAGGCTGTTGCTGCATGACAAG |
| | GACAGGATGACGGTTCACACCGGCCAGACCAACAAGTCAGCTCTTCTCTACGACTCCCTT |
| | CAAACCGAGAGTGTCCCTTTTGAGGGCCTGCTGACCGAAGGCAACACCATCCGCATCGAG |
| | TTCACCTCCGACCAGGCCCCGGCGGCCTCCACCTTCAACATCCGATTTGAAGCGTTTGAG |
| | AAAGGCCACTGCTATGAGCCCTACATCCAGAATGGGAACTTCACTACATCCGACCCGACC |
| | TATAACATTGGGACTATAGTGGAGTTCACCTGCGACCCCGGCCACTCCCTGGAGCAGCGC |
| | CCGGCCATCATCGAATGCATCAATGTGCGGGACCCATACTGGAATGACACAGAGCCCCTG |
| | TGCAGAGCCATGTGTGGTGGGCAGCTCTCTGCTGTGGCTCGGGTGGTATTGTCCCCAAAC |
| | TGGCCCGAGCCCTACGTGGAAGGTGAAGATTGTATCTGGAAGATCCACGTGGGAGAAGAG |
| | AAACGGATCTTCTTAGATATCCAGTTCCTGAATCTGAGCAACAGTGACATCTTGACCATC |
| | TACGATGGCGACGAGGTCATGCCCCACATCTTGGGGCAGTACCTTGGGAACAGTGGCCCC |
| | CAGAAACTGTACTCCTCCACGCCAGACTTAACCATCCAGTTCCATTCGGACCCTGCTGGC |
| | CTCATCTTTGGAAAGGGCCAGGGATTTATCATCAACTACATACAGGTATCAAGGAATGAC |
| | TCCTGCTCGGATTTACCCGAGATCCAGAATGCCTGGAAAACCACTTCTCACACGGAGTTG |
| | GTGCGGGGAGCCAGAATCACCTACCAGTCTGACCCCGCCTATGACATCGTGGGGAGTGAC |
| | ACCCTCACCTGCCAGTGGGACCTCAGCTGGAGCAGCGACCCCCCATTTTGTGAGAAAATT |
| | ATGTACTGCACCGACCCCGGAGAGGTGGATCACTCGACCCGCTTAATTTCGCATCCTGTG |
| | CTGCTGGTGGGGACCACCATCCAATACACCTGCAACCCCGGTTTTGTGCTTGAAGGGAGT |
| | TCTCTTCTCACCTGCTACAGCCGTGAAACAGGGACTCCCATCTGGACGTCTCGCCTGCCC |
| | CACTGCGTTTCCGAGGACTCCCTGGCATGTGACAACCCAGGGCTGCCTGAAAATGGATAC |
| | CAAATCCTGTACAAGCGACTCTACCTGCCAGCAGAGTCCCTCACCTTCATGTGCTACCAA |
| | GGCTTTGAGCTCATGGGTGAAGTGACCATCCGCTGCATCCTGGGACAGCCATCCCACTGG |
| | AACCGGCCCCTGCCCGTGTGTAAAGTTAATCAAGACACTTTTGAACATGCTTTAGAAGTA |
| | GCAGAAGCGGCACCAGAGACGTCGCTGGAAGGGGGGAACATGGCCCTGGCTATCTTCATC |
| | CCGGTCCTCATCATCTCCTTACTGCTGGGAGGAGCCTACATTTACATCACAAGATGTCGC |
| | TACTATTCCAACCTCCGCCTCCCTCTGATGTACTCCCACCCCTACAGCCAGATCACCGTG |
| | GAAACCGAGTTTGACAACCCCATTTACGAGACAGGGGAAACCAGAGAGTATGAGGTTTCT |
| | ATCTAAAGAGAGCTACACTT |

| | | |
|---|---|---|
| ORF Start: ATG at 13 | | ORF Stop: TAA at 2404 |
| SEQ ID NO: 230 | 797 aa | MW at 88285.1 kD |

| | |
|---|---|
| NOV38b, | MAQEAPQEDTSPMALMDKGENELTCSASEESQETTTSTIITTTVITTEQAPALCSVSFSN |
| CG97012-02 | |
| Protein Sequence | PEGYIDSSDYPLLPLNNFLECTYNVTVYTCYGVELQVKSVNLSDGELLSIRGVDGPTLTV |
| | LANQTLLVEGQVIRSPTNTISVYFRTFQDDGLGTFQLHYQAFMLSCNFPRRPDSCDVTVM |
| | DLHSGGVAHFHCHLGYELQGAKMLTCINASKPHWSSQEPICSAPCGGAVHNATIGRVLSP |
| | SYPENTNGSQFCIWTIEAPEGQKLHLHFERLLLHDKDRMTVHSGQTNKSALLYDSLQTES |
| | VPFEGLLSEGNTIRIEFTSDQARAASTFNIRFEAFEKGHCYEPYIQNGNFTTSDPTYNIG |
| | TIVEFTCDPGHSLEQGPAIIECINVRDPYWNDTEPLCRAMCGGELSAVAGVVLSPNWPEP |
| | YVEGEDCIWKINVGEEKRIFLDIQFLNLSNSDILTIYDGDEVMPHILGQYLGNSGPQKLY |
| | SSTPDLTIQFHSDPAGLIFGKGQGFIMNYIEVSRNDSCSDLPEIQNGWKTTSHTELVRGA |
| | RITYQCDPGYDIVGSDTLTCQWDLSWSSDPPFCEKIMYCTDPGEVDHSTRLISDPVLLVG |
| | TTIQYTCNPGFVLEGSSLLTCYSRETGTPIWTSRLPHCVSEESLACDNPGLPENGYQILY |
| | KRLYLPGESLTFMCYEGFELMGEVTIRCILGQPSHWNGPLPVCKVNQDSFEHALEVAEAA |
| | AETSLEGGNMALAIFIPVLIISLLLGGAYIYITRCRYYSNLRLPLMYSHPYSQITVETEF |
| | DNPIYETGETREYEVSI |

| | |
|---|---|
| SEQ ID NO: 231 | 1434 bp |

| | |
|---|---|
| NOV38c, | AGATCTTGCAACTTTCCCCGCCGGCCTGACTCTGGGCATGTCACGGTGATGGACCTGCAC |
| CG97012-03 | |
| DNA Sequence | TCAGGTGGGGTGGCCCACTTTCACTGCCACCTCGGCTATGAGCTCCAGGGCGCTAAGATG |
| | CTGACATGCATCAATGCCTCCAAGCCGCACTGGAGCAGCCAGGAGCCCATCTGCTCAGCT |
| | CCTTGTGCAGGGGCAGTGCACAATGCCACCATCGGCCGCGTCCTCTCCCCAAGTTACCCT |
| | GAAAACACCAATGGGAGCCAATTCTGCATCTGGACGATTGAACCTCCAGAGGCCCAGAAG |
| | CTGCACCTGCACTTTGAGAGGCTGTTCCTGCATGACAAGGACAGGATGACGCTTCACAGC |
| | GGGCAGACCAACAAGTCAGCTCTTCTCTACGACTCCCTTCAAACCGAGAGTGTCCCTTTT |
| | GAGGGCCTGCTGAGCGAAGGCAACACCATCCGCATCGAGTTCACGTCCGACCAGGCCCGG |
| | GCGGCCTCCACCTTCAACATCCGATTTGAACCGTTTGAGAAAGGCCACTGCTATCAGCCC |
| | TACATCCAGAATGGGAACTTCACTACATCCGACCCGACCTATAACATTGGGACTATAGTG |
| | GAGTTCACCTGCGACCCCGGCCACTCCCTGGAGCAGGCCCCGGCCATCATCGAATGCATC |
| | AATGTGCGGGACCCATACTGGAATGACACAGAGCCCCTGTGCAGAGCCATGTGTGGTGGG |
| | GAGCTCTCTGCTCTGGCTGGGGTGGTATTGTCCCCAAACTGGCCCGAGCCCTACGTGGAA |
| | GGTGAAGATTGTATCTGGAACATCCACGTGCGAGAAGAGAAACGGATCTTCTTAGATATC |
| | CACTTCCTGAATCTGAGCAACAGTGACATCTTGACCATCTACGATGGCCACCAGGTCATG |
| | CCCCACATCTTGCGGCAGTACCTTGGGAACAGTGGCCCCCAGAAACTGTACTCCTCCACG |
| | CCAGACTTAACCATCCAGTTCCATTCGGACCCTGCTGGCCTCATCTTTGCAAAGGGCCAG |
| | GGATTTATCATGAACTACATAGAGGTATCAAGGAATGACTCCTGCTCGGATTTACCCGAG |
| | ATCCACAATGGCTGGAAAACCACTTCTCACACGGAGTTGCTGCGCGGAGCCAGAATCACC |
| | TACCAGTGTGACCCCGGCTATGACATCGTGGGGAGTGACACCCTCACCTGCCAGTGGGAC |
| | CTCAGCTCGAGCAGCGACCCCCCATTTTGTGAGAAAACGGAGGAGTCCCTGGCATGTGAC |
| | AACCCAGGGCTGCCTGAAAATGGATACCAAATCCTGTACAAGCCACTCTACCTGCCAGGA |
| | GAGTCCCTCACCTTCATGTGCTACGAACGCTTTGAGCTCATGCGTGAAGTGACCATCCGC |
| | TGCATCCTGGGACAGCCATCCCACTGGAACGGCCCCCTGCCCGTGTGTGTCGAC |

| | | |
|---|---|---|
| ORF Start: at 7 | | ORF Stop: at 1429 |
| SEQ ID NO: 232 | 474 aa | MW at 52744.6 kD |

| | |
|---|---|
| NOV38c, | CNFPRRPDSGDVTVMDLHSGGVAHFHCHLGYELQGAKMLTCINASKPHWSSQEPICSAPC |
| CG97012-03 | |
| Protein Sequence | GGAVHNATIGRVLSPSYPENTNGSQFCIWTIEAPEGQKLHLHFERLLLHDKDRMTVHSGQ |
| | TNKSALLYDSLQTESVPFEGLLSEGNTIRIEFTSDQARAASTFNIRFEAFEKGHCYEPYI |
| | QNGNFTTSDPTYNIGTIVEFTCDPGHSLEQGPAIIECINVRDPYWNDTEPLCRAMCGGEL |
| | SAVAGVVLSPNWPEPYVECEDCIWKIHVGEEKRIFLDIQFLNLSNSDILTIYDGDEVMPH |
| | ILGQYLGNSGPQKLYSSTPDLTIQFHSDPAGLIFGKCQGFIMNYIEVSRNDSCSDLPEIQ |
| | NGWKTTSHTELVRGARITYQCDPGYDIVGSDTLTCQWDLSWSSDPPFCEKTEESLACDNP |
| | GLPENGYQTLYKRLYLPGESLTFMCYEGFELMGEVTIRCILGQPSHWNGPLPVC |

| | |
|---|---|
| SEQ ID NO: 233 | 3116 bp |

| | |
|---|---|
| NOV38d, | ATGCCCGCCGCCCCGCCCCCCGCCCCCGGCCTGCGGGGCATCACCCTGTTCCTGCCCCTG |
| CG97012-01 | |
| DNA Sequence | CTGCTGGGCAGCCCCGCCGCCGCCCTGGAGCGGCACGCCCTGCCCCAGGCCGACGCCAGC |
| | CCCCTGGGCCCCTACCTGCTGCCCAGCGGCGCCCCCGAGCGCGGCAGCCCCGGCAACCAG |
| | CACCCCGAGGAGCGGGTGGTGACCGCCCCCCCCAGCACCAGCCAGAGCGCCGAGGTGCTG |
| | GGCGACCTGGTGCTGGACGGCACCGCCCCCAGCGCCCACCACCACATCCCCGCCCTGAGC |
| | CCCCTGCTGCCCCACGAGGCCCGGCCCAAGCACCCCCTGCCCCCCAAGAAGAAGCTGCCC |
| | AGCCTGAAGCAGGTGAACAGCGCCCGGAAGCAGCTGCCGCCCAAGGCCACCAGCGCCGCC |
| | ACCGTGCAGCGGGCCCGCAGCCAGCCCGCCACCCAGGGCCTGGACCTGCTGAGCAGCAGC |
| | ACCGAGAAGCCCGCCCCCCCCGGCGACCCCGACCCCATCGTCGCCAGCGAGCAGGCCAGC |
| | GAGGTGCCCCTGTCGCTCGACCGGAAGGAGAGCGCCGTGCCCACCACCCCCCCACCCCTC |
| | CAAATCTCCCCCTTCACTTCGCAGCCCTATGTGGCCCACACACTCCCCCAGAGGCCAGAA |
| | CCCGGGGAGCCTGGGCCTGACATGGCCCAGGAGGCCCCCCAGGAGGACACCAGCCCCATG |
| | GCCCTGATGGACAAAGGTGAGAATGAGCTGACTGGGTCAGCCTCAGAGGAGAGCCAGGAG |
| | ACCACTACCTCCACCATTATCACCACCACGGTCATCACCACCGACCAGCCACCACCTCTC |
| | TGCAGTGTGAGCTTCTCCAATCCTGAGGGGTACATTGACTCCAGCGACTACCCACTGCTG |
| | CCCCTCAACAACTTTCTGGAGTGCACATACAACGTGACAGTCTACACTGGCTATCCGGTG |
| | GAGCTCCAGGTGAAGAGTGTGAACCTGTCCGATGGGCAACTGCTCTCCATCCGCGGGGTG |
| | GACGGCCCTACCCTGACCGTCCTGGCCAACCAGACACTCCTGCTGGAGGGGCAGGTAATC |
| | CGAAGCCCCACCAACACCATCTCCGTCTACTTCCGGACCTTCCAGGACGACGGCCTTGGG |
| | ACCTTCCAGCTTCACTACCAGGCCTTCATGCTCAGCTGCAACTTTCCCCGCCGGCCTGAC |
| | TCTGGGGATGTCACGGTGATGGACCTGCACTCAGGTGGCGTGGCCCACTTTCACTGCCAC |
| | CTGGGCTATGAGCTCCAGGGCGCTAAGATGCTGACATCCATCAATGCCTCCAAGCCGCAC |
| | TGGAGCAGCCAGGAGCCCATCTGCTCAGCTCCTTGTCGAGGGGCAGTGCACAATGCCACC |
| | ATCGGCCGCGTCCTCTCCCCAAGTTACCCTGAAAACACCAATGGGAGCCAATTCTGCATC |
| | TGGACGATTGAAGCTCCAGAGGGCCAGAAGCTGCACCTGCACTTTGAGAGGCTGTTGCTG |
| | CATGACAAGGACAGGATGACGGTTCACAGCGGGCAGACCAACAAGTCAGCTCTTCTCTAC |
| | GACTCCCTTCAAACCGAGAGTGTCCCTTTTGAGGGCCTGCTGAGCGAAGGCAACACCATC |
| | CGCATCGAGTTCACGTCCGACCAGGCCCGGGCGGCCTCCACCTTCAACATCCGATTTGAA |
| | GCCTTTGAGAAAGGCCACTGCTATGACCCCTACATCCACAATGGGAACTTCACTACATCC |
| | GACCCGACCTATAACATTGGGACTATAGTGGAGTTCACCTGCGACCCCGGCCACTCCCTG |
| | GAGCAGGGCCCGGCCATCATCGAATCCATCAATGTGCGGGACCCATACTGGAATGACACA |
| | GAGCCCCTGTGCAGAGCCATGTGTGGTGGGGAGCTCTCTGCTGTGGCTGGGGTGGTATTG |
| | TCCCCAAACTGGCCCGAGCCCTACGTGGAACGTGAAGATTGTATCTGGAAGATCCACGTG |
| | GGAGAAGAGAAACCGATCTTCTTAGATATCCAGTTCCTGAATCTGAGCAACAGTGACATC |
| | TTGACCATCTACGATGGCGACGAGGTCATGCCCCACATCTTGGGGCACTACCTTGGGAAC |
| | AGTGGCCCCCAGAAACTGTACTCCTCCACGCCAGACTTAACCATCCAGTTCCATTCGCAC |
| | CCTGCTGGCCTCATCTTTGGAAAGGGCCAGGGATTTATCATGAACTACATAGAGCTATCA |
| | AGGAATGACTCCTGCTCGGATTTACCCGAGATCCAGAATGGCTGGAAAACCACTTCTCAC |
| | ACGGAGTTGGTCCCGGGAGCCAGAATCACCTACCAGTGTGACCCCGGCTATCACATCGTC |
| | GGGAGTGACACCCTCACCTCCCAGTGGGACCTCACCTGGAGCAGCGACCCCCCATTTTGT |
| | CAGAAAATTATGTACTGCACCGACCCCGGAGAGGTGGATCACTCGACCCGCTTAATTTCG |
| | GATCCTCTGCTGCTCGTGGGGACCACCATCCAATACACCTGCAACCCCGGTTTTGTGCTT |
| | GAAGGCAGTTCTCTTCTGACCTCCTACAGCCGTGAAACAGCGACTCCCATCTGGACGTCT |
| | CGCCTGCCCCACTCCGTTTCGGAGGAGTCCCTGGCATGTGACAACCCAGGCCTCCCTGAA |
| | AATGGATACCAAATCCTGTACAACCGACTCTACCTGCCACGAGAGTCCCTCACCTTCATG |
| | TGCTACCAAGGCTTTGAGCTCATGGGTGAAGTGACCATCCGCTGCATCCTGGGACACCCA |
| | TCCCACTGGAACGGGCCCCTGCCCGTGTGTAAAGTAGCAGAAGCGGCAGCAGAGACGTCG |
| | CTGGAAGGGGGGAACATGGCCCTGGCTATCTTCATCCCGCTCCTCATCATCTCCTTACTG |
| | CTGCGAGGAGCCTACATTTACATCACAAGATGTCCCTACTATTCCAACCTCCGCCTGCCT |
| | CTGATGTACTCCCACCCCTACAGCCAGATCACCGTGGAAACCGAGTTTGACAACCCCATT |
| | TACGAGACAGGGGAAACCAGAGACTATGAGGTTTCTATCTAAAGAGAGCTACACTTGAGA |
| | AGGCGACTTGTGAACTCAACCACAATCTCCTCGAGACATTCATCCAGAGACCATGT |

| | | |
|---|---|---|
| ORF Start: ATG at 1 | | ORF Stop: TAA at 3040 |
| SEQ ID NO: 234 | 1013 aa | MW at 110509.9 kD |

| | |
|---|---|
| NOV38d, | MPAARPPAAGLRCISLFLALLLGSPAAALERDALPEGDASPLGPYLLPSGAPERGSPGKE |
| C697012-01 | |
| Protein Sequence | HPEERVVTAPPSSSQSAEVLGELVLDGTAPSAHHDIPALSPLLPEEARPKHALPPKKKLP |
| | SLKQVNSARKQLRPKATSAATVQRAGSQPASQGLDLLSSSTEKPGPPGDPDPIVASEEAS |
| | EVPLWLDRKESAVPTTPAFLQTSPFTSQPYVAHTLPQRPEPGEPGPDMAQEAPQEDTSPM |
| | ALMDKCENELTGSASEESQETTTSTITTTTVITTEQAPALCSVSFSNPEGYIDSSDYPLL |
| | PLNNFLECTYNVTVYTGYGVELQVKSVNLSDGELLSIRGVDGPTLTVLANQTLLVEGQVI |
| | RSPTNTISVYFRTFQDDGLGTFQLHYQAFMLSCNFPRRPDSGDVTVMDLHSCGVAHFHCH |
| | LGYELQGAKMLTCINASKPHWSSQEPICSAPCCGAVHNATIGRVLSPSYPENTNCSQFCI |
| | WTIEAPEGQKLHLHFERLLLHDKDRMTVHSGQTNKSALLYDSLQTESVPFEGLLSECNTI |
| | RIEFTSDQARAASTFNIRFEAFEKGHCYEPYIQNGNFTTSDPTYNIGTIVEFTCDPGHSL |
| | EQGPAITECINVRDPYWNDTEPLCRAMCGGELSAVAGVVLSPNWPEPYVEGEDCIWKIHV |
| | GEEKRTFLDIQFLNLSNSDILTIYDGDEVMPHILCQYLGNSGPQKLYSSTPDLTTQFHSD |
| | PAGLIFCKGQGFIMNYIEVSRNDSCSDLPEIQNGWKTTSHTELVRGARITYQCDPGYDIV |
| | GSDTLTCQWDLSWSSDPPFCEKIMYCTDPGEVDHSTRLISDPVLLVGTTTQYTCNPGFVL |
| | EGSSLLTCYSRETGTPIWTSRLPHCVSEESLACDNPGLPENGYQILYKRLYLPGESLTFM |
| | CYEGFELMGEVTIRCILGQPSHWNGPLPVCKVAEAAAETSLEGGNMALAIFIPVLIISLL |
| | LGGAYIYITRCRYYSNLRLPLMYSHPYSQITVETEFDNPIYETGETREYEVSI |

| | |
|---|---|
| SEQ ID NO: 235 | 867 bp |

| | |
|---|---|
| NOV38e, | AGATCTTGTGGACGGGCAGTGCACAATGCCACCATCGGCCGCGTCCTCTCCCCAAGTTAC |
| 210120300 | |
| DNA Sequence | CCTGAAAACACCAATCGGAGCCAATTCTGCATCTGGACGATTGAAGCTCCAGACGGCCAG |
| | AAGCTGCACCTGCACTTTGAGACGCTGTTGCTGCATGACAAGGACAGGATGACGGTTCAC |
| | AGCGGGCAGACCAACAAGTCAGCTCTTCTCTACGACTCCCTTCAAACCGAGAGTGTCCCT |
| | TTTGAGGCCCTGCTGAGCGAAGGCAACACCATCCGCATCGAGTTCACGTCCGACCAGGCC |
| | CGGGCGGCCTCCACCTTCAACATCCGATTTGAAGCGTTTGAGAAAGGCCACTGCTATGAG |
| | CCCTACATCCAGAATGGGAACTTCACTACATCCGACCCGACCTATAACATTGGGACTATA |
| | GTGGAGTTCACCTGCGACCCCGGCCACTCCCTGGAGCAGGGCCCGGCCATCATCGAATGC |
| | ATCAATGTGCGGGACCCATACTGGAATGACACAGAGCCCCTGTGCAGAGCCATGTGTGGT |
| | GGGGAGCTCTCTGCTGTGGCTCCGGTGGTATTGTCCCCAAACTGGCCCGAGCCCTACGTG |
| | GAAGGTGAAGATTGTATCTGCAAGATCCACGTGGGAGAAGAGAAACGGATCTTCTTAGAT |
| | ATCCAGTTCCTGAATCTGAGCAACAGTGACATCTTGACCATCTACGATGGCGACGAGGTC |
| | ATGCCCCACATCTTGGGGCAGTACCTTGCCAACAGTGGCCCCCAGAAACTGTACTCCTCC |
| | CAGGGATTTATCATGAACTACGTCGAC |

| | | |
|---|---|---|
| ORF Start: at 1 | | ORF Stop: end of sequence |
| SEQ ID NO: 236 | 289 aa | MW at 32172.6 kD |

| | |
|---|---|
| NOV38e, | RSCGGAVNNATIGRVLSPSYPENTNGSQFCIWTIEAPEGQKLHLHFERLLLHDKDRMTVH |
| 210120300 | |
| Protein Sequence | SGQTNKSALLYDSLQTESVPFEGLLSECNTIRIEFTSDQARAASTFNIRFEAFEKGHCYE |
| | PYIQNCNFTTSDPTYNIGTIVEFTCDPGHSLEQGPAIIECINVRDPYWNDTEPLCRAMCG |
| | GELSAVAGVVLSPNWPEPYVEGEDCIWKIHVGEEKRIFLDIQFLNLSNSDILTIYDGDEV |
| | MPHILGQYLGNSCPQKLYSSTPDLTIQFHSDPAGLIFGKGQGFIMNYVD |

| | |
|---|---|
| SEQ ID NO: 237 | 867 bp |

| | |
|---|---|
| NOV38f | AGATCTTGTGCAGGGCCAGTGCACAATGCCACCATCGGCCGCGTCCTCTCCCCAAGTTAC |
| 210120376 | |
| DNA Sequence | CCTGAAAACACAAATGGGAGCCAATTCTGCATCTGGACGATTGAAGCTCCAGAGGGCCAG |
| | AAGCTGCACCTGCACTTTGAGAGGCTGTTGCTCCATCACAAGCACAGGATGACGGTTCAC |
| | AGCGGGCAGACCAACAAGTCAGCTCTTCTCTACGACTCCCTTCAAACCGAGACTGTCCCT |
| | TTTGAGGGCCTGCTGAGCCAAGGCAACACCATCCCCATCGAGTTCACGTCCGACCAGGCC |
| | CGGGCGGCCTCCACCTTCAACATCCCATTTGAAGCGTTTGACAAAGGCCACTGCTATGAG |
| | CCCTACATCCAGAATGGGAACTTCACTACATCCGACCCGACCTATAACATTGGGACTATA |
| | GTGGAGTTCACCTGCCACCCCGGCCACTCCCTGGAGCAGGCCCCGGCCATCATCGAATGC |
| | ATCAATGTGCGGGACCCATACTGCAATGACACACACCCCCTGTGCAGAGCCATGTGTGGT |
| | GGGCAGCTCTCTGCTGTGGCTGGGGTGCTATTGTCCCCAAACTGGCCCGAGCCCTACGTG |
| | GAAGGTGAAGATTGTATCTGGAAGATCCACGTGGGAGAAGAGAAACGGATCTTCTTACAT |
| | ATCCAGTTCCTGAATCTGAGCAACAGTGACATCTTGACCATCTACGATGGCGACGACGTC |
| | ATGCCCCACATCTTCGGGCAGTACCTTGGGAACAGTCGCCCCCAGAAACTGTACTCCTCC |
| | ACGCCAGACTTAACCATCCAGTTCCATTCGCACCCTGCTGGCCTCATCTTTGGAAAGGGC |
| | CAGGGATTTATCATGAACTACGTCGAC |

| | | |
|---|---|---|
| ORF Start: at 1 | | ORF Stop: end of sequence |
| SEQ ID NO: 238 | 289 aa | MW at 32172.6 kD |

| | |
|---|---|
| NOV38f, | RSCGGAVHNATIGRVLSPSYPENTNGSQFCIWTTEAPEGQKLHLHFERLLLHDKDRMTVH |
| 210120376 | |
| Protein Sequence | SGQTNKSALLYDSLQTESVPFEGLLSEGNTTRIEFTSDQARAASTFNIRFEAPEKGHCYE |
| | PYIQNGNFTTSDPTYNIGTIVEFTCDPGHSLEQGPAIIECINVRDPYWNDTEPLCRANCG |
| | GELSAVAGVVLSPNWPEPYVEGEDCIWKIHVGEEKRIFLDIQFLNLSNSDILTIYDGDEV |
| | MPHILGQYLGNSGPQKLYSSTPDLTIQFHSDPAGLIFGKCQGPIMNYVD |

| | |
|---|---|
| SEQ ID NO: 239 | 867 bp |

| | |
|---|---|
| NOV38g, | AGATCTTGTGGAGGGGCAGTGCACAATGCCACCATCGGCCGCCTCCTCTCCCCAAGTTAC |
| 210120463 | |
| DNA Sequence | CCTGAAAACACCAATGGGAGCCAATTCTCCATCTGGACGATTGAAGCTCCACAGGCCCGG |
| | AAGCTGCACCTGCACTTTGAGAGGCTGTTGCTGCATGACAAGGACAGGATGACGGTTCAC |
| | ACCCGGCAGACCAACAAGTCAGCTCTTCTCTACGACTCCCTTCAAACCGAGAGTGTCCCT |
| | TTTGAGGGCCTGCTGAGCGAAGGCAACACCATCCGCATCCAGTTCACGTCCGACCAGGCC |
| | CGGGCGGCCTCCACCTTCAACATCCGATTTGAAGCGTTTGAGAAAGGCCACTGCTATGAC |
| | CCCTACATCCAGAATGGGAACTTCACTACATCCCACCCGACCTATAACATTGGGACTATA |
| | GTGGAGTTCACCTGCOACCCCGGCCACTCCCTGCACCAGCGCCCGGCCATCATCGAATGC |
| | ATCAATGTGCGGGACCCATACTGGAATGACACAGAGCCCCTGTGCAGAGCCATCTGTGGT |
| | GGCGAGCTCTCTGCTGTGGCTGGGGTGGTATTCTCCCCAAACTGGCCCGAGCCCTACGTC |
| | GAAGGTGAAGATTGTATCTGGAAGATCCACGTGGGAGAAGAGAAACGGATCTTCTTACAT |
| | ATCCAGTTCCTCAATCTGAGCAACAGTGACATCTTGACCATCTACGATGGCGACGAGGTC |
| | ATGCCCCACATCTTGGGGCAGTACCTTGGGAACAGTGGCCCCCAGAAACTGTACTCCTCC |
| | ACGCCAGACTTAACCATCCAGTTCCATTCCGACCCTGCTGGCCTCATCTTTGGAAAGGGC |
| | CAGGGATTTATCATGAACTACGTCGAC |

| | | |
|---|---|---|
| ORF Start: at 1 | | ORF Stop: end of sequence |
| SEQ ID NO: 240 | 289 aa | MW at 32200.7 kD |

| | |
|---|---|
| NOV38g, | RSCGGAVHNATIGRVLSPSYPENTNGSQFCIWTIEAPEGRKLHLHFERLLLHDKDRNTVH |
| 210120463 | |
| Protein Sequence | SGQTNKSALLYDSLQTESVPFEGLLSEGNTIRIEFTSDQARAASTFNIRFEAFEKGHCYE |
| | PYIQNGNFTTSDPTYNIGTIVEFTCDPGHSLEQGPAIIECINVRDPYWNDTEPLCRAMCG |
| | GELSAVAGVVLSPNWPEPYVEGEDCIWKIHVGEEKRIFLDIQFLNLSNSDILTIYDGDEV |
| | MPHILGQYLGNSGPQKLYSSTPDLTIQFHSDPAGLIFGKGQGFIMNYVD |

| | |
|---|---|
| SEQ ID NO: 241 | 1434 bp |

| | |
|---|---|
| NOV38h, | AGATCTTGCAACTTTCCCCGCCGGCCTGACTCTGCGGATGTCACGGTGATGGACCTGCAC |
| 210120269 | |
| DNA Sequence | TCAGGTGGGGTGGCCCACTTTCACTGCCACCTGGGCTATGAGCTCCAGGGCGCTAAGATG |
| | CTGACATGCATCAATGCCTCCAAGCCCCACTGGAGCAGCCAGGAGCCCATCTGCTCAGCT |
| | CCTTGTGGAGGGGCAGTGCACAATGCCACCATCGGCCGCGTCCTCTCCCCAAGTTACCCT |
| | GAAAACACCAATGGGAGCCAATTCTGCATCTGGACGATTGAAGCTCCAGAGGGCCAGAAG |
| | CTGCACCTGCACTTTGAGAGGCTCTTGCTGCATGACAAGGACAGGATGACGGTTCACAGC |
| | GGGCAGACCAACAAGTCAGCTCTTCTCTACGACTCCCTTCAAACCGAGAGTGTCCCTTTT |
| | GAGGGCCTGCTGACCGAAGGCAACACCATCCGCATCGAGTTCACGTCCGACCAGGCCCGG |
| | GCGGCCTCCACCTTCAACATCCGATTTGAAGCGTTTCAGAAAGGCCACTGCTATGAGCCC |
| | TACATCCAGAATGGGAACTTCACTACATCCGACCCGACCTATAACATTGGGACTATACTG |
| | GAGTTCACCTGCGACCCCGCCCACTCCCTGGAGCACGGCCCGGCCATCATCGAATGCATC |
| | AATGTGCGGGACCCATACTGGAATGACACAGAGCCCCTGTGCAGAGCCATGTGTGGTGGG |
| | GAGCTCTCTGCTCTGGCTGGGGTGGTATTGTCCCCAAACTGGCCCCAGCCCTACGTGGAA |
| | GGTGAAGATTGTATCTGGAAGATCCACGTGGCAGAAGACAAACGGATCTTCTTACATATC |
| | CAGTTCCTGAATCTGAGCAACAGTGACATCTTGACCATCTACGATGGCGACCAGGTCATG |
| | CCCCACATCTTGGGGCAGTACCTTGGGAACAGTGGCCCCCAGAAACTGTACTCCTCCACG |
| | CCAGACTTAACCATCCAGTTCCATTCGGACCCTGCTGGCCTCATCTTTGGAAACGCCCAG |
| | GGATTTATCATGAACTACATAGAGGTATCAAGGAATGACTCCTGCTCGGATTTACCCGAG |
| | ATCCAGAATGGCTGGAAAACCACTTCTCACACGGAGTTGGTGCGGGGAGCCAGAATCACC |
| | TACCAGTGTGACCCCGGCTATGACATCGTCGGGAGTGACACCCTCACCTGCCAGTGGGAC |
| | CTCAGCTGGAGCAGCGACCCCCCATTTTGTGAGAAAACGGAGGAGTCCCTGGCATGTGAC |
| | AACCCAGGGCTGCCTGAAAATGGATACCAAATCCTGTACAAGCGACTCTACCTGCCAGGA |
| | GAGTCCCTCACCTTCATGTGCTACGAAGGCTTTGAGCTCATGGGTGAAGTGACCATCCGC |
| | TGCATCCTCGGACAGCCATCCCACTGGAACGGGCCCCTGCCCCTCTGTCTCGAC |

| | | |
|---|---|---|
| ORF Start: at 1 | | ORF Stop: end of sequence |
| SEQ ID NO: 242 | 478 aa | MW at 53202.0 kD |

| | |
|---|---|
| NOV38h, | RSCNFPRRPDSGDVTVMDLHSGGVAHFHCHLGYELQGAKMLTCINASKPHWSSQEPICSA |
| 210120269 | |
| Protein Sequence | PCGGAVHNATIGRVLSPSYPENTNGSQFCIWTIEAPECQKLHLHFERLLLHDKDRMTVHS |
| | GQTNKSALLYDSLQTESVPFEGLLSEGNTIRTEFTSDQARAASTFNIRFEAFEKGHCYEP |
| | YIQNGNFTTSDPTYNIGTIVEFTCDPGHSLEQGPAIIECINVRDPYWNDTEPLCRANCGG |
| | ELSAVAGVVLSPNWPEPYVEGEDCIWKIHVGEEKRIFLDIQFLNLSNSDILTIYDGDEVM |
| | PHILGQYLGNSGPQKLYSSTPDLTIQFHSDPAGLIFGKGQGFIMNYIEVSRNDSCSDLPE |
| | IQNCWKTTSHTELVRGARITYQCDPGYDIVGSDTLTCQWDLSWSSDPPFCEKTEESLACD |
| | NPGLPENGYQILYKRLYLPGESLTFMCYEGPELMGEVTTRCILGQPSHWNGPLPVCVD |

| | |
|---|---|
| SEQ ID NO: 243 | 867 bp |

| | |
|---|---|
| NOV38i, | AGATCTTGTGGAGGGGCAGTGCACAATGCCACCATCGGCCGCCTCCTCTCCCCAAGTTAC |
| CG97012-04 | |
| DNA Sequence | CCTGAAAACACCAATGGGAGCCAATTCTGCATCTGGACGATTGAAGCTCCAGAGGGCCAG |
| | AAGCTGCACCTGCACTTTGAGAGGCTCTTGCTCCATGACAAGCACAGGATCACGCTTCAC |
| | AGCGGGCAGACCAACAAGTCAGCTCTTCTCTACGACTCCCTTCAAACCGAGAGTGTCCCT |
| | TTTGAGGGCCTGCTGAGCGAACGCAACACCATCCGCATCGAGTTCACGTCCGACCAGGCC |
| | CGGGCGGCCTCCACCTTCAACATCCGATTTGAAGCGTTTGAGAAAGGCCACTGCTATGAG |
| | CCCTACATCCAGAATGGCAACTTCACTACATCCGACCCGACCTATAACATTGGGACTATA |
| | GTGGAGTTCACCTGCGACCCCGGCCACTCCCTGGACCAGGGCCCGGCCATCATCGAATGC |
| | ATCAATGTGCCGGACCCATACTCGAATGACACAGAGCCCCTGTGCAGAGCCATGTGTGGT |
| | GGGGAGCTCTCTGCTGTGGCTGGGGTGGTATTGTCCCCAAACTGGCCCGAGCCCTACGTG |
| | GAAGGTGAAGATTGTATCTGGAAGATCCACGTGGGAGAAGAGAAACGGATCTTCTTAGAT |
| | ATCCACTTCCTGAATCTGAGCAACAGTGACATCTTCACCATCTACGATGGCGACGAGGTC |
| | ATGCCCCACATCTTGGGGCAGTACCTTGGGAACAGTGGCCCCCAGAAACTGTACTCCTCC |
| | ACGCCAGACTTAACCATCCAGTTCCATTCGGACCCTGCTGCCCTCATCTTTGGAAAGGGC |
| | CAGGGATTTATCATGAACTACGTCGAC |

| | | |
|---|---|---|
| ORF Start: at 7 | | ORF Stop: at 862 |
| SEQ ID NO: 244 | 285 aa | MW at 31715.2 kD |

| | |
|---|---|
| NOV38i, | CGGAVHNATIGRVLSPSYPENTNGSQFCIWTIEAPEGQKLHLHFERLLLHDKDRMTVUSG |
| CG97012-04 | |
| Protein Sequence | QTNKSALLYDSLQTESVPFEGLLSEGNTIRIEFTSDQARAASTFNIRFEAFEKGHCYEPY |
| | IQNGNFTTSDPTYNIGTIVEFTCDPGHSLEQCPAIIECINVRDPYWNDTEPLCRAMCGGE |
| | LSAVAGVVLSPNWPEPYVEGEDCIWKIHVGEEKRIFLDIQFLNLSNSDILTIYDGDEVMP |
| | HILGQYLGNSGPQKLYSSTPDLTIQFHSDPAGLIFGKGQGFIMNY |

| | |
|---|---|
| SEQ ID NO: 245 | 2861 bp |

| | |
|---|---|
| NOV38j, | AGCCACCATGCCCGCGGCCCGGCCGCCCGCCGCGGGACTCCGCGGGATCTCGCTGTTCCT |
| CG97012-05 | |
| DNA Sequence | CGCTCTGCTCCTGGCGAGCCCGGCGGCAGCGCTGGAGCCAGATGCTCTTCCCCAGGGAGA |
| | TGCTAGCCCTTTGGGTCCTTACCTCCTGCCCTCAGGAGCCCCGGAGAGAGGCAGTCCTCG |
| | CAAAGAGCACCCTGAAGAGAGAGTGGTAACAGCGCCCCCCAGTTCCTCACAGTCGGCGGA |
| | AGTGCTGGGCGAGCTGGTGCTGGATGGGACCGCACCCTCTGCACATCACGACATCCCAGC |
| | CCTGTCACCGCTGCTTCCAGAGGAGGCCCGCCCCAAGCACGCCTTGCCCCCCAAGAAGAA |
| | ACTGCCTTCGCTCAAGCAGGTCAACTCTGCCAGCAAGCAGCTGAGGCCCAAGGCCACCTC |
| | CGCAGCCACTCTCCAAACGGCAGGGTCCCAGCCAGCGTCCCAGGGCCTACATCTCCTCTC |
| | CTCCTCCACGGAGAAGCCTCGCCCACCGCGGGACCCGGACCCCATCGTGGCCTCCGAGGA |
| | GGCATCAGAAGTGCCCCTTTGGCTGGATCGAAAGGAGAGTGCGGTCCCTACAACACCCGC |
| | ACCCCTGCAAATCTCCCCCTTCACTTCGCAGCCCTATGTGGCCCACACACTCCCCCACAG |
| | GCCACAACCCGGGGAGCCTGGGCCTGACATGGCCCAGGAGGCCCCCCAGGACGACACCAG |
| | CCCCATGGCCCTGATGGACAAAGGTGAGAATGAGCTGACTGGGTCAGCCTCACAGGAGAG |
| | CCAGGAGACCACTACCTCCACCATTATCACCACCACGGTCATCACCACCGAGCAAGCACC |
| | AGCTCTCTGCAGTGTGAGCTTCTCCAATCCTGAGGGGTACATTGACTCCAGCGACTACCC |
| | ACTGCTGCCCCTCAACAACTTTCTGGAGTGCACATACAACGTGACAGTCTACACTGGCTA |
| | TCGGGTGGAGCTCCAGGTGAAGAGTGTGAACCTGTCCGATGGGGAACTGCTCTCCATCCC |
| | CGGGGTGGACCGCCCTACCCTGACCGTCCTGGCCAACCAGACACTCCTCGTGGAGGGGCA |
| | GGTAATCCGAAGCCCCACCAACACCATCTCCGTCTACTTCCGGACCTTCCAGGACCACGG |
| | GCCTGACTCTGGGCATGTCACGGTGATCCACCTGCACTCAGGTGGGGTCGCCCACTTTCA |
| | CTGCCACCTCGGCTATGAGCTCCAGGGCGCTAAGATGCTGACATCCATCAATGCCTCCAA |
| | GCCGCACTGGAGCAGCCAGCAGCCCATCTGCTCAGCTCCTTGTGGAGGGGCAGTGCACAA |
| | TGCCACCATCGGCCGCGTCCTCTCCCCAAGTTACCCTGAAAACACCAATGGGAGCCAATT |
| | CTTGCTGCATGACAAGGACAGGATGACGGTTCACAGCGCCCAGACCAACAAGTCAGCTCT |
| | TCTCTACGACTCCCTTCAAACCGAGAGTGTCCCTTTTGAGGGCCTGCTGAGCGAAGGCAA |
| | ATTTGAAGCGTTTGAGAAAGGCCACTGCTATGAGCCCTACATCCAGAATGGGAACTTCAC |
| | TACATCCGACCCGACCTATAACATTGCGACTATAGTGGAGTTCACCTCCCACCCCGGCCA |
| | CTCCCTGGAQCAGGCCCCGGCCATCATCGAATCCATCAATGTGCGGGACCCATACTGGAA |
| | TCACACACAGCCCCTGTCCACACCCATGTGTCGTGGGCACCTCTCTGCTGTGGCTGGGGT |
| | GGTATTGTCCCCAAACTCGCCCGAGCCCTACGTGGAAGCTGAACATTGTATCTGGAAGAT |
| | CCACGTGGCACAAGACAAACGGATCTTCTTAGATATCCAGTTCCTGAATCTGAGCAACAC |
| | TGACATCTTCACCATCTACCATGGCCACCAGGTCATGCCCCACATCTTGCGCCAGTACCT |
| | TGGGAACAGTGGCCCCCAGAAACTGTACTCCTCCACGCCAGACTTAACCATCCAGTTCCA |
| | TTCGGACCCTCCTGCCCTCATCTTTGGAAAGCGCCAGGGATTTATCATGAACTACATAGA |
| | GGTATCAAGGAATGACTCCTGCTCGGATTTACCCGAGATCCAGAATGGCTGGAAAACCAC |
| | TTCTCACACGGAGTTGGTGCGGGGACCCAGAATCACCTACCAGTGTCACCCCGGCTATGA |
| | CATCGTGGGGAGTGACACCCTCACCTGCCAGTGGGACCTCAGCTGGAGCAGCGACCCCCC |
| | ATTTTCTGAGAAAACGGAGGAGTCCCTGCCATGTGACAACCCACGGCTGCCTGAAAATCG |
| | ATACCAAATCCTGTACAAGCCACTCTACCTGCCAGGAGAGTCCCTCACCTTCATCTGCTA |
| | CCAACGCTTTGACCTCATCGGTCAAGTGACCATCCGCTGCATCCTGGGACAGCCATCCCA |
| | CTGGAACGGGCCCCTGCCCGTGTCTAAAGTAGCAGAAGCGGCAGCAGAGACCTCGCTGCA |
| | AGGGGGGAACATGGCCCTGCCTATCTTCATCCCGGTCCTCATCATCTCCTTACTGCTGGG |
| | AGGAGCCTACATTTACATCACAAGATCTCGCTACTATTCCAACCTCCGCCTGCCTCTCAT |
| | GTACTCCCACCCCTACAGCCAGATCACCGTGGAAACCGAGTTTGACAACCCCATTTACCA |
| | GACAGGGGAAACCAGAGAGTATGAGGTTTCTATCTAAAGAG |

| | | |
|---|---|---|
| ORF Start: ATG at 8 | | ORF Stop: TAA at 2855 |
| SEQ ID NO: 246 | 949 aa | MW at 103496.0 kD |

| | |
|---|---|
| NOV38j, | MPAARPPAAGLRGISLFLALLLGSPAAALERDALPEGDASPLGPYLLPSGAPERGSPGKE |
| CG97012-05 | |
| Protein Sequence | HPEERVVTAPPSSSQSAEVLGELVLDGTAPSAHHDIPALSPLLPEEARPKHALPPKKKLP |
| | SLKQVNSARKQLRPKATSAATVQRAGSQPASQGLDLLSSSTEKPGPPGDPDPIVASEEAS |
| | EVPLWLDRKESAVPTTPAPLQISPFTSQPYVAHTLPQRPEPGEPGPDMAQEAPQEDTSPM |
| | ALMDKGENELTGSASEESQETTTSTIITTTVITTEQAPALCSVSFSNPEGYIDSSDYPLL |
| | PLNNFLECTYNVTVYTGYGVELQVKSVNLSDGELLSIRGVDGPTLTVLANQTLLVEGQVI |
| | RSPTNTISVYFRTFQDDGLGTFQLHYQAFMLSCNFPRRPDSGDVTVMDLHSGGVAHFHCH |
| | LGYELQGAKNLTCINASKPHWSSQEPICSAPCGGAVHNATIGRVLSPSYPENTNGSQFCI |
| | WTIEAPEGQKLHLHFERLLLHDKDRMTVHSGQTNKSALLYDSLQTESVPFEGLLSEGNTI |
| | RIEFTSDQARAASTFNIRFEAFEKGHCYEPYIQNGNFTTSDPTYNIGTIVEFTCDPGHSL |
| | EQGPAIIECINVRDPYWNDTEPLCRANCGGELSAVAGVVLSPNWPEPYVEGEDCIWKIHV |
| | GEEKRIFLDIQFLNLSNSDILTIYDGDEVMPHILGQYLGNSGPQKLYSSTPDLTIQFHSD |
| | PAGLIFGKGQGFIMNYIEVSRNDSCSDLPEIQNGWKTTSHTELVRGARITYQCDPGYDIV |
| | GSDTLTCQWDLSWSSDPPFCEKTEESLACDNPGLPENGYQILYKRLYLPGESLTFMCYEG |
| | FELMGEVTIRCILGQPSHWNGPLPVCKVAEAAAETSLEGGNMALAIFIPVLIISLLLGCA |
| | YIYITRCRYYSNLRLPLMYSHPYSQITVETEFDNPIYETGETREYEVSI |

Sequence comparison of the above protein sequences yields the following sequence relationships shown in Table 38B.

TABLE 38B
Comparison of NOV38a against NOV38b through NOV38j.

| | NOV38a Residues/ | Identities/Similarities |
|---|---|---|
| Protein Sequence | Match Residues | for the Matched Region |
| NOV38b | 228 . . . 1013 | 751/797 (94%) |
| | 1 . . . 797 | 752/797 (94%) |
| NOV38c | 393 . . . 865 | 427/477 (89%) |
| | 1 . . . 474 | 439/477 (91%) |
| NOV38d | 30 . . . 1013 | 944/984 (95%) |
| | 30 . . . 1013 | 944/984 (95%) |
| NOV38e | 452 . . . 738 | 285/287 (99%) |
| | 3 . . . 289 | 287/287 (99%) |
| NOV38f | 452 . . . 738 | 285/287 (99%) |
| | 3 . . . 289 | 287/287 (99%) |
| NOV38g | 452 . . . 738 | 284/287 (98%) |
| | 3 . . . 289 | 287/287 (99%) |
| NOV38h | 392 . . . 866 | 429/479 (89%) |
| | 2 . . . 477 | 441/479 (91%) |
| NOV38i | 452 . . . 736 | 285/285 (100%) |
| | 1 . . . 285 | 285/285 (100%) |
| NOV38j | 30 . . . 872 | 752/847 (88%) |
| | 30 . . . 873 | 765/847 (89%) |

Two polymorphic variants of NOV38a have been identified and are shown in Table 41P. Further analysis of the NOV38a protein yielded the following properties shown in Table 38C.

TABLE 38C
Protein Sequence Properties NOV38a

| | |
|---|---|
| PSort | 0.6760 probability located in plasma membrane; |
| analysis: | 0.1800 probability located innucleus; 0.1000 |
| | probability located in endoplasmic reticulum |
| | (membrane);0.1000 probability located in |
| | endoplasmic reticulum (lumen) |
| SignalP | Cleavage site between residues 29 and 30 |
| analysis: | |

A search of the NOV38a protein against the Geneseq database, a proprietary database that contains sequences published in patents and patent publication, yielded several homologous proteins shown in Table 38D.

TABLE 38D
Geneseq Results for NOV38a

| | | NOV38a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU12271 | Human PRO6094 | 1 . . . 1013 | 1013/1023 (99%) | 0.0 |
| | polypeptide sequence - | 1 . . . 1023 | 1013/1023 (99%) | |
| | Homo sapiens, 1023 aa. | | | |
| | [WO200140466-A2, 07 | | | |
| | JUN. 2001] | | | |
| ABG22405 | Novel human diagnostic | 29 . . . 1013 | 983/985 (99%) | 0.0 |
| | protein #22396 - Homo | 6 . . . 990 | 984/985 (99%) | |
| | sapiens, 990 aa. | | | |
| | [WO200175067-A2, 11 | | | |
| | OCT. 2001] | | | |
| ABG05922 | Novel human diagnostic | 29 . . . 1013 | 983/985 (99%) | 0.0 |
| | protein #5913 - Homo | 6 . . . 990 | 984/985 (99%) | |
| | sapiens, 990 aa. | | | |
| | [WO200175067-A2, 11 | | | |
| | OCT. 2001] | | | |
| ABG01221 | Novel human diagnostic | 33 . . . 1013 | 981/981 (100%) | 0.0 |
| | protein #1212 - Homo | 2 . . . 982 | 981/981 (100%) | |
| | sapiens, 982 aa. | | | |
| | [WO200175067-A2, 11 | | | |
| | OCT. 2001] | | | |
| ABG22407 | Novel human diagnostic | 29 . . . 1008 | 967/991 (97%) | 0.0 |
| | protein #22398 - Homo | 6 . . . 996 | 971/991 (97%) | |
| | sapiens, 997 aa. | | | |
| | [WO200175067-A2, 11 | | | |
| | OCT. 2001] | | | |

In a BLAST search of public sequence datbases, the NOV38a protein was found to have homology to the proteins shown in the BLASTP data in Table 38E.

TABLE 38E
Public BLASTP Results for NOV38a

| | | NOV38a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9BYH1 | Seizure 6-like protein | 1 . . . 1013 | 1013/1024 (98%) | 0.0 |
| | precursor - Homo sapiens | 1 . . . 1024 | 1013/1024 (98%) | |
| | (Human), 1024 aa. | | | |
| Q9Y2E1 | KIAA0927 protein - Homo | 1 . . . 872 | 821/876 (93%) | 0.0 |
| | sapiens (Human), 1001 aa | 53 . . . 925 | 834/876 (94%) | |
| | (fragment). | | | |
| Q9Y3J6 | Hypothetical 87.6 kDa | 228 . . . 1008 | 778/791 (98%) | 0.0 |
| | protein (DJ268D13.1.2) | 1 . . . 791 | 780/791 (98%) | |
| | (seizure related gene 6 | | | |
| | (mouse)-like (KIAA0927) | | | |
| | (isoform 2)) - Homo sapiens | | | |
| | (Human), 792 aa. | | | |
| Q9NUI3 | DJ268D13.1.3 (Seizure | 228 . . . 1004 | 775/779 (99%) | 0.0 |
| | related gene 6 (Mouse)-like | 1 . . . 777 | 775/779 (99%) | |
| | (KIAA0927) (Isoform 3)) - | | | |
| | Homo sapiens (Human), 777 | | | |
| | aa (fragment). | | | |
| O95917 | Hypothetical 79.0 kDa | 228 . . . 868 | 641/641 (100%) | 0.0 |
| | protein (DJ268D13.1.1) | 1 . . . 641 | 641/641 (100%) | |
| | (seizure related gene 6 | | | |
| | (mouse)-like (KIAA0927) | | | |
| | (isoform 1)) - Homo sapiens | | | |
| | (Human), 716 aa. | | | |

PFam analysis predicts that the NOV38a protein contains the domains shown in Table 38F.

TABLE 38F
Domain Analysis of NOV38a

| | | Identities/ | |
|---|---|---|---|
| | | Similarities | |
| Pfam | NOV38a | for the | Expect |
| Domain | Match Region | Matched Region | Value |
| sushi | 393 . . . 448 | 16/65 (25%) | 6e−06 |
| | | 41/65 (63%) | |
| CUB | 452 . . . 559 | 29/120 (24%) | 5.5e−09 |
| | | 72/120 (60%) | |
| sushi | 567 . . . 624 | 19/67 (28%) | 4.5e−06 |
| | | 44/67 (66%) | |
| CUB | 628 . . . 736 | 34/121 (28%) | 1.6e−15 |
| | | 69/121 (57%) | |
| sushi | 745 . . . 800 | 22/64 (34%) | 1.3e−14 |
| | | 44/64 (69%) | |
| sushi | 806 . . . 865 | 21/66 (32%) | 3.2e−11 |
| | | 47/66 (71%) | |
| sushi | 873 . . . 930 | 20/65 (31%) | 4e−12 |
| | | 47/65 (72%) | |

### Example 39

The NOV39 clone was analyzed, and the nucleotide and encoded polypeptide sequences are shown in Table 39A.

TABLE 39A
NOV39 Sequence Analysis

| | |
|---|---|
| SEQ ID NO: 247 | 1957 bp |

| | |
|---|---|
| NOV39a, | CAGCTGAGCAACAGGATGCTGGCGGGGGCCGTGACGAGCATGCCCACCCCCCTCCTCCCC |
| CG99754-01 | |
| DNA Sequence | TGCTGGCAGCCCATCCTCCTGCTGCTGCTGGGCTCAGTGCTGTCAGGCTCGGCCACGCGC |
| | TCCCCGCCCCGCTGCGAGTGCTCCGCCCAGGACCGCCCTGTGCTGTGCCACCGCAAGCGC |
| | TTTGTGGCAGTCCCCGAGGGCATCCCCACCGAGACGCGCCTGCTCGACCTACGCAAGAAC |
| | CGCATCAAAACGCTCAACCAGGACGACTTCGCCAGCTTCCCGCACCTGGAGCAGCTGCAG |
| | CTCAACGAGAACATCGTGAGCGCCCTGCAGCCCCGCGCCTTCAACAACCTCTTCAACCTC |
| | CGGACGCTGGGTCTCCGCAGCAACCGCCTCAAGCTCATCCCGCTAGGCGTCTTCACTCGC |
| | CTCAGCAACCTGACCAAGCTGGACATCAGCGAGAACAAGATCGTTATCCTACTGGACTAC |
| | ATGTTTCAGGACCTGTACAACCTCAAGTCACTGGAGGTTGGCGACAATGACCTCGTCTAC |
| | ATCTCTCACCGCGCCTTCAGCGGCCTCAACAGCCTCGACCAGCTGACGCTGGAGAAATGC |
| | AACCTGACCTCCATCCCCACCGAGGCGCTGTCCCACCTCCACGGCCTCATCCTCCTGAGG |
| | CTCCGCCACCTCAACATCAATGCCATCCGCGACTACTCCTTCAAGAGGCTGTACCGACTC |
| | AAGGTCTTGCAGATCTCCCACTGGCCCTACTTGGACACCATCACACCCAACTGCCTCTAC |
| | GGCCTCAACCTGACGTCCCTGTCCATCACACACTGCAATCTGACCGCTGTGCCCTACCTC |
| | GCCCTCCGCCACCTAGTCTATCTCCGCTTCCTCAACCTCTCCTACAACCCCATCAGCACC |
| | ATTGACGGCTCCATGTTGCATCAGCTGCTCCGCCTGCACGAGATCCACCTGGTCGGCGGG |
| | CAGCTGGCCGTGCTGGAGCCCTATGCCTTCCGCGGCCTCAACTACCTGCGCGTGCTCAAT |
| | GAGACACTCATCCTGGACTCCAACCCCCTCGCCTGCGACTCTCGGCTCCTGTGGGTCTTC |
| | CGGCGCCGCTCGCGGCTCAACTTCAACCCCCAGCAGCCCACGTGCGCCACGCCCGAGTTT |
| | GTCCAGGGCAAGGAGTTCAAGGACTTCCCTGATGTGCTACTGCCCAACTACTTCACCTGC |
| | CGCCGCCCCCGCATCCGCGACCGCAAGGCCCAGCACGTCTTTGTCGACGAGGGCCACACC |
| | GTGCAGTTTGTGTGCCGGGCCCATGGCGACCCCCCGCCCGCCATCCTCTCGCTCTCACCC |
| | CGAAAGCACCTGGTCTCACCCAAGAGCAATGGGCGGCTCACAGTCTTCCCTGATGGCACG |
| | CTGGAGGTGCGCTACGCCCAGGTACAGGACAACGGCACGTACCTGTGCATCGCGGCCAAC |
| | GCGGGCGGCAACCACTCCATGCCCGCCCACCTGCATGTGCGCAGCTACTCGCCCCACTGG |
| | AACACCACCCGCGCCACTGTGCCTTTCCCCTTCGACATCAAGACCCTCATCATCGCCACC |
| | ACCATGGGCTTCATCTCTTTCCTGGGCCTCGTCCTCTTCTGCCTCGTGCTCCTGTTTCTC |
| | TGGAGCCGGGCCAAGGGCAACACAAAGCACAACATCGAGATCGAGTATGTGCCCCGAAAG |
| | TCGGACCCAGGCATCAGCTCCGCCGACGCGCCCCCCAAGTTCAACATGAAGATGATATGA |
| | GGCCGGGGCGGGGGGCAGGGACCCCCGGGCGGCCGGCCAGGGGAAGGGGCCTCGCCGCCA |
| | CCTGCTCACTCTCCAGTCCTTCCCACCTCCTCCCTAC |

| | | |
|---|---|---|
| ORF Start: ATG at 16 | | ORF Stop: TGA at 1858 |
| SEQ ID NO: 248 | 614 aa | MW at 69145.1 kD |

| | |
|---|---|
| NOV39a, | MLAGGVRSMPSPLLACWQPILLLVLGSVLSGSATGCPPRCECSAQDRAVLCHRKRFVAVP |
| CG99754-01 | |
| Protein Sequence | EGIPTETRLLDLGKNRIKTLNQDEFASFPHLEELELNENIVSAVEPCAFNNLFNLRTLGL |
| | RSNRLKLIPLGVFTGLSNLTKLDISENKIVILLDYMFQDLYNLKSLEVGDNDLVYISHRA |
| | FSGLNSLEQLTLEKCNLTSIPTEALSHLHGLIVLRLRHLNINAIRDYSFKRLYRLKVLEI |
| | SHWPYLDTMTPNCLYGLNLTSLSITHCNLTAVPYLAVRHLVYLRFLNLSYNPISTIEGSM |
| | LHELLRLQEIQLVGGQLAVVEPYAFRGLNYLRVLNVSGNQLTTLEESVFHSVGNLETLIL |
| | DSNPLACDCRLLWVFRRRWRLNFNRQQPTCATPEFVQGKEFKDFPDVLLPNYFTCRRARI |
| | RDRKAQQVFVDEGHTVQFVCRADGDPPPAILWLSPRKHLVSAKSNGRLTVFPDGTLEVRY |
| | AQVQDNGTYLCIAANAGGNDSMPAHLHVRSYSPDWPHQPNKTFAPISNQPGEGEANSTRA |
| | TVPFPFDIKTLIIATTMGFISFLGVVLFCLVLLFLWSRGKGNTKHNIEIEYVPRKSDAGI |
| | SSADAPRKFNMKMI |

| | |
|---|---|
| SEQ ID NO: 249 | 2015 bp |

| | |
|---|---|
| NOV39b, | GAGCTGAGGCTGCTGGGGGGCGTGAGGAGCATGCCCAGCCCCCTCCTGGCCTGCTCGCAG |
| CG99754-02 | |
| DNA Sequence | CCCATCCTCCTGCTGGTGCTGGGCTCAGTGCTGTCAGGCTCGGCCACGGGCTGCCCGCCC |
| | CCCTGCCAGTGCTCCGCCCAGGACCGCCCTGTGCTGTGCCACCGCAAGCGCTTTGTGGCA |
| | GTCCCCGAGGGCATCCCCACCGAGACGCGCCTGCTGGACCTAGGCAAGAACCGCATCAAA |
| | ACGCTCAACCAGGACGAGTTCGCCAGCTTCCCGCACCTGGAGGAGCTGGAGCTCAACGAG |
| | AACATCGTCAGCGCCGTGGAGCCCGGCGCCTTCAACAACCTCTTCAACCTCCGGACGCTG |
| | GGTCTCCGCAGCAACCCCCTGAAGCTCATCCCGCTAGGCGTCTTCACTGGCCTCAGCAAC |
| | CTGACCAAGCTGGACATCAGCGAGAACAAGATCGTTATCCTACTGGACTACATGTTTCAG |
| | GACCTGTACAACCTCAAGTCACTGCAGGTTGGCGACAATGACCTCGTCTACATCTCTCAC |
| | CGCGCCTTCAGCGGCCTCAACAGCCTGGAGCACCTGACGCTGGAGAAATGCAACCTGACC |
| | TCCATCCCCACCGAGGCGCTGTCCCACCTGCACGGCCTCATCGTCCTGAGGCTCCGGCAC |
| | CTCAACATCAATGCCATCCGGGACTACTCCTTCAAGAGGCTGTACCGACTCAAGGTCTTG |
| | CTGACGTCCCTGTCCATCACACACTGCAATCTGACCGCTGTGCCCTACCTGGCCGTCCCC |
| | CACCTAGTCTATCTCCGCTTCCTCAACCTCTCCTACAACCCCATCAGCACCATTGAGGGC |
| | TCCATGTTCCATGACCTGCTCCGCCTGCAGGAGATCCAGCTGCTGGGCGGGCACCTCCCC |
| | GTGGTGGAGCCCTATGCCTTCCGCGGCCTCAACTACCTGCCCGTGCTCAATGTCTCTGGC |
| | AACCAGCTGACCACACTGGACGAATCAGTCTTCCACTCGGTGGGCAACCTGGAGACACTC |
| | TGCCGGCTCAACTTCAACCGGCAGCAGCCCACGTGCGCCACGCCCGAGTTTGTCCACGGC |
| | AAGGAGTTCAAGGACTTCCCTGATGTGCTACTGCCCAACTACTTCACCTGCCGCCGCGCC |
| | CGCATCCGGGACCGCAAGGCCCAGCAGGTGTTTGTGGACGAGGGCCACACGGTGCAGTTT |
| | GTGTGCCGGCCCGATGCCGACCCGCCGCCCGCCATCCTCTGGCTCTCACCCCGAAAGCAC |
| | CTGGTCTCACCCAACAGCAATCGGCGCCTCACAGTCTTTCCTGATGGCACGCTGCAGCTG |
| | CGCTACGCCCAGGTACAGGACAACGGCACGTACCTCTGCATCGCGGCCAACGCGGGCGCC |
| | AACGACTCCATGCCCGCCCACCTGCATGTGCGCAGCTACTCGCCCCACTGGCCCCATCAC |
| | CCCAACAAGACCTTCGCTTTCATCTCCAACCAGCCGCGCGAGGGAGAGCCCAACAGCACC |
| | CGCGCCACTGTGCCTTTCCCCTTCGACATCAAGACCCTCATCATCGCCACCACCATGCGC |
| | TTCATCTCTTTCCTGGCCGTCGTCCTCTTCTGCCTGGTGCTGCTGTTTCTCTGGAGCCGG |
| | GGCAAGGGCAACACAAACCACAACATCGAGATCGAGTATGTGCCCCAAAAGTCGGACGCA |
| | GGCATCAGCTCCGCCGACGCGCCCCGCAAGTTCAACATGAAGATGATATGAGCCCGGGGC |
| | GGGGGGCAGGCACCCCCGGGCGGCCGGGCAGGGGAAGGGGCCTCGCCGCCACCTGCTCAC |
| | TCTCCAGTCCTTCCCACCTCCTCCCTACCCTTCTACACACGTTCTCTTTCTCCCTCCCGC |
| | CTCCGTCCCCTGCTGCCCCCCACCACCCTCAGCTC |

| | | |
|---|---|---|
| ORF Start: ATG at 31 | | ORF Stop: TGA at 1849 |
| SEQ ID NO: 250 | 606 aa | MW at 68345.1 kD |

| | |
|---|---|
| NOV39b, | MPSPLLACWQPILLLVLGSVLSGSATGCPPRCECSAQDRAVLCHRKRFVAVPECIPTETR |
| CG99754-02 | |
| Protein Sequence | LLDLGKNRIKTLNQDEFASFPHLEELELNENIVSAVEPGAFNNLFNLRTLGLRSNRLKLI |
| | PLGVFTCLSNLTKLDISENKIVILLDYMFQDLYNLKSLEVGDNDLVYISHRAFSGLNSLE |
| | QLTLEKCNLTSIPTEALSHLHGLIVLRLRHLNINAIRDYSFKRLYRLKVLEISHWPYLDT |
| | MTPNCLYGLNLTSLSITHCNLTAVPYLAVRHLVYLRFLNLSYNPISTIEGSMLHELLRLQ |
| | EIQLVGGQLAVVEPYAFRGLNYLRVLNVSGNQLTTLEESVFHSVGNLETLILDSNPLACD |
| | CRLLWVFRRRWRLNFNRQQPTCATPEFVQGKEFKDFPDVLLPNYFTCRRARIRDRKAQQV |
| | FVDEGHTVQFVCRADGDPPPAILWLSPRKHLVSAKSNGRLTVFPDGTLEVRYAQVQDNGT |
| | YLCIAANAGGNDSMPAHLHVRSYSPDWPHQPNKTFAFISNQPGEGEANSTRATVPFPFDI |
| | KTLIIATTMGFISFLCVVLFCLVLLFLWSRGKGNTKHNIEIEYVPQKSDAGISSADAPRK |
| | FNMKMI |

Sequence comparison of the above protein sequences yields the following sequence relationships shown in Table 39B.

TABLE 39B
Comparison of NOV39a against NOV39b.

| | | Identities/Similarities |
|---|---|---|
| Protein | NOV39a Residues/ | for the |
| Sequence | Match Residues | Matched Region |
| NOV39b | 9 . . . 614 | 563/606 (92%) |
| | 1 . . . 606 | 564/606 (92%) |

Six polymorphic variants of NOV39a have been identified and are shown in Table 41Q. Further analysis of the NOV39a protein yielded the following properties shown in Table 39C.

TABLE 39C
Protein Sequence Properties NOV39a

| | |
|---|---|
| PSort | 0.4600 probability located in plasma membrane; |
| analysis: | 0.1071 probability located inmicrobody (peroxisome); |
| | 0.1000 probability located in endoplasmic reticulum |
| | (membrane); 0.1000 probability located in |
| | endoplasmic reticulum (lumen) |
| SignalP | Cleavage site between residues 36 and 37 |
| analysis: | |

A search of the NOV39a protein against the Geneseq database, a proprietary database that contains sequences published in patents and patent publication, yielded several homologous proteins shown in Table 39D.

TABLE 39D
Geneseq Results for NOV39a

| | | NOV39a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAB74705 | Human membrane associated | 1 . . . 614 | 614/614 (100%) | 0.0 |
| | protein MEMAP-11 - Homo | 7 . . . 620 | 614/614 (100%) | |
| | sapiens, 620 aa. | | | |
| | [WO200112662-A2, 22 | | | |
| | FEB. 2001] | | | |
| AAW84596 | Amino acid sequence of the | 1 . . . 614 | 612/614 (99%) | 0.0 |
| | human Tango-79 protein - | 1 . . . 614 | 612/614 (99%) | |
| | Homo sapiens, 614 aa. | | | |
| | [WO9906427-A1, 11 FEB. | | | |
| | 1999] | | | |
| AAB80225 | Human PRO227 protein - | 1 . . . 614 | 612/614 (99%) | 0.0 |
| | Homo sapiens, 620 aa. | 7 . . . 620 | 612/614 (99%) | |
| | [WO200104311-A1, 18 | | | |
| | JAN. 2001] | | | |
| AAU12333 | Human PRO227 polypeptide | 1 . . . 614 | 612/614 (99%) | 0.0 |
| | sequence - Homo sapiens, | 7 . . . 620 | 612/614 (99%) | |
| | 620 aa. [WO200140466-A2, | | | |
| | 07 JUN. 2001] | | | |
| AAY13357 | Amino acid sequence of | 1 . . . 614 | 612/614 (99%) | 0.0 |
| | protein PRO227 - Homo | 7 . . . 620 | 612/614 (99%) | |
| | sapiens, 620 aa. | | | |
| | [WO9914328-A2, 25 MAR. | | | |
| | 1999] | | | |

In a BLAST search Of public sequence datbases, the NOV39a protein was found to have homology to the proteins shown in the BLASTP data in Table 39E.

TABLE 39E
Public BLASTP Results for NOV39a

| | | NOV39a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q96FE5 | Unknown (protein for | 1 . . . 614 | 614/614 (100%) | 0.0 |
| | MGC: 17422) - Homo | 1 . . . 614 | 614/614 (100%) | |
| | sapiens (Human), 614 aa. | | | |
| Q9N008 | Hypothetical 69.2 kDa | 1 . . . 614 | 612/614 (99%) | 0.0 |
| | protein - Macaca fascicularis | 1 . . . 614 | 613/614 (99%) | |
| | (Crab eating macaque) | | | |
| | (Cynomolgus monkey), 614 | | | |
| | aa. | | | |
| Q9D1T0 | Adult male testis cDNA, | 1 . . . 614 | 610/614 (99%) | 0.0 |
| | RIKEN full-length enriched | 1 . . . 614 | 611/614 (99%) | |
| | library, clone: 4930471K13, | | | |
| | full insert sequence - Mus | | | |
| | musculus (Mouse), 614 aa. | | | |
| CAD38935 | Hypothetical protein - Homo | 38 . . . 614 | 577/577 (100%) | 0.0 |
| | sapiens (Human), 577 aa | 1 . . . 577 | 577/577 (100%) | |
| | (fragment). | | | |
| Q9BZ20 | BA438B23.1 (Neuronal | 14 . . . 614 | 365/603 (60%) | 0.0 |
| | leucine-rich repeat protein) | 6 . . . 606 | 468/603 (77%) | |
| | (CDNA FLJ31810 fis, clone | | | |
| | NT2RI2009289, weakly | | | |
| | similar to carboxypeptidase | | | |
| | N 83 kDa chain) - Homo | | | |
| | sapiens (Human), 606 aa. | | | |

PFam analysis predicts that the NOV39a protein contains the domains shown in Table 39F.

TABLE 39F
Domain Analysis of NOV39a

| | | Identities/ | |
|---|---|---|---|
| | | Similarities | |
| Pfam | NOV39a | for the Matched | Expect |
| Domain | Match Region | Region | Value |
| LRRNT | 35 . . . 64 | 10/31 (32%) | 0.00079 |
| | | 22/31 (71%) | |
| LRR | 114 . . . 137 | 9/25 (36%) | 0.061 |
| | | 20/25 (80%) | |
| LRR | 186 . . . 209 | 10/25 (40%) | 0.012 |
| | | 19/25 (76%) | |
| LRR | 282 . . . 305 | 7/25 (28%) | 0.72 |
| | | 17/25 (68%) | |
| LRR | 330 . . . 353 | 7/25 (28%) | 0.19 |
| | | 20/25 (80%) | |
| LRRCT | 363 . . . 416 | 17/59 (29%) | 0.0021 |
| | | 39/59 (66%) | |
| ig | 433 . . . 493 | 15/64 (23%) | 2.1e−09 |
| | | 44/64 (69%) | |

### Example 40

The NOV40 clone was analyzed, and the nucleotide and encoded polypeptide sequences are shown in Table 40A.

TABLE 40A
NOV40 Sequence Analysis

| | |
|---|---|
| SEQ ID NO: 251 | 889 bp |

| | |
|---|---|
| NOV40a, | GGGAGAATCCTTCTTGGAACAGAGATGGGCCCAGAACTGAATCAGATGAAGAGAGATAAG |
| CG99777-01 | |
| DNA Sequence | GTGTGATGTGGGGAAGACTATATAAAGAATGGACCCAGGGCTGCAGCAAGCACTCAACGG |
| | AATGGCCCCTCCTGGAGACACACCCATGCATGTGCCGGCGGCCTCCGTGGCCAGCCACCT |
| | GGGCACCACGAGCCGCAGCTATTTCTATTTGACCACAGCCACTCTGGCTCTGTGCCTTGT |
| | CTTCACGGTGGCCACTATTATGGTGTTGGTCGTTCAGAGGACGGACTCCATTCCCAACTC |
| | ACCTGACAACGTCCCCCTCAAAGGAGGAAATTGCTCAGAAGACCTCTTATGTATCCTGAA |
| | CAAGTTGTCTTGGAACAAAGATGGCATTCTCCATGGAGTCAGATATCAGGATGGGAATCT |
| | GGTGATCCAATTCCCTGGTTTGTACTTCATCATTTGCCAACTGCAGTTTCTTGTACAATG |
| | CCCAAATAATTCTGTCGATCTGAAGTTGGAGCTTCTCATCAACAAGCATATCAAAAAACA |
| | GGCCCTGGTGACAGTCTGTGAGTCTGGAATGCAAACGAAACACGTATACCAGAATCTCTC |
| | TCAATTCTTGCTGGATTACCTGCAGGTCAACACCACCATATCAGTCAATGTGGATACATT |
| | CCAGTACATAGATACAAGCACCTTTCCTCTTGAGAATGTGTTGTCCATCTTCTTATACAG |
| | TAATTCAGACTGAACAGTTTCTCTTGGCCTTCAGGAAGAAAGCGCCTCTCCACCATACAC |
| | TATTTCATCCCTCCAAACACTTGGGCAAAAAGAAAACTTTAGACCAAGA |

| | | |
|---|---|---|
| ORF Start: ATG at 89 | | ORF Stop: TGA at 791 |
| SEQ ID NO: 252 | 234 aa | MW at 26016.9 kD |

| | |
|---|---|
| NOV40a, | MDPGLQQALNGMAPPGDTANHVPAGSVASHLGTTSRSYFYLTTATLALCLVFTVATIMVL |
| CG99777-01 | |
| Protein Sequence | VVQRTDSIPNSPDNVPLKGGNCSEDLLCILKRAPFKKSWAYLQVAKHLNKTKLSWNKDGI |
| | LHGVRYQDGNLVIQFPGLYFIICQLQFLVQCPNNSVDLKLELLINKHIKKQALVTVCESG |
| | MQTKHVYQNLSQFLLDYLQVNTTISVNVDTFQYIDTSTFPLENVLSIFLYSNSD |

| | |
|---|---|
| SEQ ID NO: 253 | 829 bp |

| | |
|---|---|
| NOV40b, | GGGAGAATCCTTCTTGGAACAGAGATGGGCCCAGAACTGAATCAGATGAAGAGAGATAAG |
| CG99777-02 | |
| DNA Sequence | GTGTGATGTGGGGAAGACTATATAAAGAATGGACCCAGGGCTGCAGCAAGCACTCAACGG |
| | AATGGCCCCTCCTGGAGACACAGCCATGCATGTGCCGGCGGGCTCCGTGGCCAGCCACCT |
| | GGGGACCACGAGCCGCACCTATTTCTATTTGACCACAGCCACTCTGGCTCTGTGCCTTGT |
| | CTTCACGGTGGCCACTATTATGGTGTTGGTCGTTCAGAGGACGGACTCCATTCCCAACTC |
| | ACCTGACAACGTCCCCCTCAAAGGAGTGGCAAAGCATCTAAACAAAACCAAGTTGTCTTG |
| | GAACAAACATGGCATTCTCCATGGAGTCAGATATCAGGATGGGAATCTGGTGATCCAATT |
| | CCCTGGTTTGTACTTCATCATTTGCCAACTGCAGTTTCTTGTACAATGCCCAAATAATTC |
| | TGTCGATCTGAAGTTGGAGCTTCTCATCAACAAGCATATCAAAAAACAGGCCCTGGTGAG |
| | AGTGTGTGAGTCTGGAATGCAAACGAAACACGTATACCAGAATCTCTCTCAATTCTTGCT |
| | GGATTACCTGCAGGTCAACACCACCATATCAGTCAATCTGGATACATTCCAGTACATAGA |
| | TACAAGCACCTTTCCTCTTGAGAATGTGTTGTCCATCTTCCTATACAGTAATTCAGACTG |
| | AACAGTTTCTCTTGGCCTTCAGGAAGAAAGCGCCTCTCTACCATACAGTATTTCATCCCT |
| | CCAAACACTTGGCCAAAAAGAAAACTTTAGACCAAGAAGGATTCTCCTC |

| | | |
|---|---|---|
| ORF Start: ATG at 89 | | ORF Stop: TGA at 719 |
| SEQ ID NO: 254 | 210 aa | MW at 23250.6 kD |

| | |
|---|---|
| NOV40b, | MDPGLQQALNGMAPPGDTAMHVPAGSVASHLGTTSRSYFYLTTATLALCLVFTVATIMVL |
| CG99777-02 | |
| Protein Sequence | VVQRTDSIPNSPDNVPLKGVAKHLNKTKLSWNKDGILHGVRYQDGNLVIQFPGLYFIICQ |
| | LQPLVQCPNNSVDLKLELLINKHTKKQALVTVCESGMQTKHVYQNLSQFLLDYLQVNTTI |
| | SVNVDTFQYIDTSTFPLENVLSIFLYSNSD |

Sequence comparison of the above protein sequences yields the following sequence relationships shown in Table 40B.

TABLE 40B
Comparison of NOV40a against NOV40b.

| | | Identities/Similarities |
|---|---|---|
| Protein | NOV40a Residues/ | for the |
| Sequence | Match Residues | Matched Region |
| NOV40b | 1 . . . 234 | 210/234 (89%) |
| | 1 . . . 210 | 210/234 (89%) |

Three polymorphic variants of NOV40b have been identified and are shown in Table 41R.

Further analysis of the NOV40a protein yielded the following properties shown in Table 40C.

TABLE 40C
Protein Sequence Properties NOV40a

| | |
|---|---|
| PSort | 0.7900 probability located in plasma membrane; |
| analysis: | 0.3000 probability located in microbody (peroxisome); |
| | 0.3000 probability located in Golgi body; 0.2000 |
| | probability located in endoplasmic reticulum (membrane) |
| SignalP | Cleavage site between residues 68 and 69 |
| analysis: | |

A search of the NOV40a protein against the Geneseq database, a proprietary database that contains sequences published in patents and patent publication, yielded several homologous proteins shown in Table 40D.

TABLE 40D
Geneseq Results for NOV40a

| | | NOV40a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAU78086 | Human CD30-ligand | 1 . . . 234 | 234/234 (100%) | e−135 |
| | (CD30L) protein sequence - | 1 . . . 234 | 234/234 (100%) | |
| | Homo sapiens, 234 aa. | | | |
| | [WO200211767-A2, 14 | | | |
| | FEB. 2002] | | | |
| AAR45009 | Sequence encoded by a | 1 . . . 234 | 234/234 (100%) | e−135 |
| | human CD30-L cDNA clone | 1 . . . 234 | 234/234 (100%) | |
| | encoding additional N- | | | |
| | terminal amino acids - Homo | | | |
| | sapiens, 234 aa. | | | |
| | [WO9324135-A, 09 DEC. | | | |
| | 1993] | | | |
| AAR45007 | Sequence encoded by a | 20 . . . 234 | 215/215 (100%) | e−123 |
| | human CD30-L cDNA clone | 1 . . . 215 | 215/215 (100%) | |
| | - Homo sapiens, 215 aa. | | | |
| | [WO9324135-A, 09 DEC. | | | |
| | 1993] | | | |
| AAU78087 | Mouse CD30-ligand | 1 . . . 234 | 167/240 (69%) | 4e−92 |
| | (CD30L) protein sequence - | 1 . . . 239 | 195/240 (80%) | |
| | Mus sp, 239 aa. | | | |
| | [WO200211767-A2, 14 | | | |
| | FEB. 2002] | | | |
| AAR45008 | Sequence encoded by a | 1 . . . 234 | 167/240 (69%) | 4e−92 |
| | murine CD30-L cDNA clone | 1 . . . 239 | 195/240 (80%) | |
| | encoding additional N- | | | |
| | terminal amino acids - | | | |
| | Acomys cahirinus, 239 aa. | | | |
| | [WO9324135-A, 09 DEC. | | | |
| | 1993] | | | |

In a BLAST search of public sequence datbases, the NOV40a protein was found to have homology to the proteins shown in the BLASTP data in Table 40E.

TABLE 40E
Public BLASTP Results for NOV40a

| | | NOV40a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P32971 | Tumor necrosis factor ligand | 1 . . . 234 | 234/234 (100%) | e−134 |
| | superfamily member 8 | 1 . . . 234 | 234/234 (100%) | |
| | (CD30 ligand) (CD30- L) | | | |
| | (CD153 antigen) - Homo | | | |
| | sapiens (Human), 234 aa. | | | |
| P32972 | Tumor necrosis factor ligand | 1 . . . 234 | 167/240 (69%) | 1e−91 |
| | superfamily member 8 | 1 . . . 239 | 195/240 (80%) | |
| | (CD30 ligand) (CD30- L) - | | | |
| | Mus musculus (Mouse), 239 | | | |
| | aa. | | | |
| AAD46392 | CD30 LIGAND- | 86 . . . 234 | 149/149 (100%) | 9e−83 |
| | EXOTOXIN A FUSION | 48 . . . 196 | 149/149 (100%) | |
| | PROTEIN - synthetic | | | |
| | construct, 220 aa (fragment). | | | |
| P41047 | Tumor necrosis factor ligand | 97 . . . 195 | 31/123 (25%) | 0.056 |
| | superfamily member 6 (FAS | 142 . . . 264 | 53/123 (42%) | |
| | antigen ligand) - Mus | | | |
| | musculus (Mouse), 279 aa. | | | |
| Q9WV90 | Fas ligand - Marmota monax | 100 . . . 154 | 20/58 (34%) | 0.49 |
| | (Woodchuck), 169 aa | 44 . . . 101 | 29/58 (49%) | |
| | (fragment). | | | |

PFam analysis predicts that the NOV40a protein contains the domains shown in Table 40F.

TABLE 40F
Domain Analysis of NOV40a

| Pfam | NOV40a | Identities/Similarities | Expect |
|---|---|---|---|
| Domain | Match Region | for the Matched Region | Value |
| TNF | 93 . . . 230 | 55/159 (35%) | 1.6e−53 |
| | | 136/159 (86%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive-panel (containing normal tissue and samples from autoimmune/autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:128s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1× TaqMan® Universal Master mix (Applied Biosystems; catalog No. 4324020), following the manufactirer's instructions. PCR amplification was performed as follows: 95° C. 10 min, then 40 cycles of 95° C. for 15 seconds, 60° C. for 1 minute. Results were analyzed and processed as described previously.

### Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=pl effusion=pleural effusion,

glio=glioma,

astro=astrocytoma, and

neuro=neuroblastoma.

### General_Screening_Panel_v1.4, v1.5 and v1.6

The plates for Panels 1.4, v1.5 and v1.6 include two control wells (genomic DNA control and chemistry control) and 94 wells containing cDNA from various samples. The samples in Panels 1.4, v1.5 and v1.6 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, v1.5 and v1.6 are widely available through the American Type Culture Collection (ATCC), a repository for cultured cell lines, and were cultured using the conditions recommended by the ATCC. The normal tissues found on Panels 1.4, v1.5 and v1.6 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

### Panels 2D, 2.2, 2.3 and 2.4

The plates for Panels 2D, 2.2, 2.3 and 2.4 generally include two control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics. The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen. General oncology screening panel_v_{—}2.4 is an updated version of Panel 2D.

### HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

### ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

### Panels 3D and 3.1

The plates of Panels 3D and 3.1 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 10.3D are of the most common cell lines used in the scientific literature. Oncology_cell_line_screening panel_v3.2 is an updated version of Panel 3. The cell lines in panel 3D, 3.1, 1.3D and oncology-cell_line_screening_panel_v3.2 are of the most common cell lines used in the scientific literature.

### Panels 4D, 4R, and 4.1D

Panel 4 includes samples on a 96 well plate (2 control wells, 94 test samples) composed of RNA (Panel 4R) or cDNA (Panels 4D/4. ID) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, MD) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 10 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1/Th2 and Tr1 cells, six-well Falcon plates were coated overnight with 10 μg/ml anti-CD28 (Pharmingen) and 2,g/ml OKT3 (ATCC), and then washed twice with PBS. Umbilical cord blood CD4 lymphocytes (Poietic Systems, German Town, MD) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 μg/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 PM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 g/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

### AI_Comprehensive Panel_v1.0

The plates for AI_comprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the tine samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-1anti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS)(BA)(MF)=Individual patients

Adj=Adjacent tissue

Match control=adjacent tissues

-M=Male

-F=Female

COPD=Chronic obstructive pulmonary disease

### Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were also obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2 Diabetic Hispanic, overweight, not on insulin

Patient 7-9 Nondiabetic Caucasian and obese (BMI>30)

Patient 10 Diabetic Hispanic, overweight, on insulin

Patient 11 Nondiabetic African American and overweight

Patient 12 Diabetic Hispanic on insulin

Adipocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2, 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 AM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the SD and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stein Cells

### Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

### Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V 1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG133274-02: Induced Myeloid Leukemia Cell

Differentiation Protein MCL-1-Like Protein.

Expression of gene CG 133274-02 was assessed using the primer-probe set Ag7050, described in Table AA. Results of the RTQ-PCR runs are shown in Table AB.

TABLE AA
Probe Name Ag7050

| | | | Start | |
|---|---|---|---|---|
| Primers | Sequences | Length | Position | SEQ ID No |
| Forward | 5′-gtctcgtggttgcgctg-3′ | 17 | 450 | 255 |
| Probe | TET-5′- | 25 | 485 | 256 |
| | tcgtaaggtctccagcgccttcctg- | | | |
| | 3′-TAMRA | | | |
| Reverse | 5′-gattggcgccaaggaca-3′ | 17 | 541 | 257 |

TABLE AB
General_screening_panel_v1.6

| | Rel. Exp. (%) |
|---|---|
| | Ag7050, Run |
| Tissue Name | 282273858 |
| Adipose | 100.0 |
| Melanoma* Hs688(A).T | 33.7 |
| Melanoma* Hs688(B).T | 34.9 |
| Melanoma* M14 | 33.0 |
| Melanoma* LOXIMVI | 49.0 |
| Melanoma* SK-MEL-5 | 22.8 |
| Squamous cell carcinoma SCC-4 | 19.2 |
| Testis Pool | 12.7 |
| Prostate ca.* (bone met) PC-3 | 44.4 |
| Prostate Pool | 18.3 |
| Placenta | 27.2 |
| Uterus Pool | 14.0 |
| Ovarian ca. OVCAR-3 | 46.3 |
| Ovarian ca. SK-OV-3 | 53.6 |
| Ovarian ca. OVCAR-4 | 32.1 |
| Ovarian ca. OVCAR-5 | 55.5 |
| Ovarian ca. IGROV-1 | 31.4 |
| Ovarian ca. OVCAR-8 | 34.4 |
| Ovary | 21.5 |
| Breast ca. MCF-7 | 72.2 |
| Breast ca. MDA-MB-231 | 60.3 |
| Breast ca. BT 549 | 81.2 |
| Breast ca. T47D | 18.2 |
| Breast ca. MDA-N | 12.3 |
| Breast Pool | 14.6 |
| Trachea | 44.1 |
| Lung | 11.5 |
| Fetal Lung | 81.2 |
| Lung ca. NCI-N417 | 15.3 |
| Lung ca. LX-1 | 61.6 |
| Lung ca. NCI-H146 | 17.8 |
| Lung ca. SHP-77 | 55.9 |
| Lung ca. A549 | 28.1 |
| Lung ca. NCI-H526 | 25.2 |
| Lung ca. NCI-H23 | 90.1 |
| Lung ca. NCI-H460 | 37.1 |
| Lung ca. HOP-62 | 27.2 |
| Lung ca. NCI-H522 | 33.9 |
| Liver | 2.7 |
| Fetal Liver | 14.1 |
| Liver ca. HepG2 | 20.4 |
| Kidney Pool | 40.9 |
| Fetal Kidney | 11.3 |
| Renal ca. 786-0 | 31.6 |
| Renal ca. A498 | 12.3 |
| Renal ca. ACHN | 31.9 |
| Renal ca. UO-31 | 33.9 |
| Renal ca. TK-10 | 59.5 |
| Bladder | 60.7 |
| Gastric ca. (liver met.) NCI-N87 | 87.1 |
| Gastric ca. KATO III | 59.0 |
| Colon ca. SW-948 | 19.8 |
| Colon ca. SW480 | 36.1 |
| Colon ca.* (SW480 met) SW620 | 25.0 |
| Colon ca. HT29 | 28.7 |
| Colon ca. HCT-116 | 56.6 |
| Colon ca. CaCo-2 | 24.0 |
| Colon cancer tissue | 69.3 |
| Colon ca. SW1116 | 12.1 |
| Colon ca. Colo-205 | 10.2 |
| Colon ca. SW-48 | 11.0 |
| Colon Pool | 14.4 |
| Small Intestine Pool | 24.5 |
| Stomach Pool | 19.3 |
| Bone Marrow Pool | 11.5 |
| Fetal Heart | 13.5 |
| Heart Pool | 13.9 |
| Lymph Node Pool | 16.0 |
| Fetal Skeletal Muscle | 8.5 |
| Skeletal Muscle Pool | 17.0 |
| Spleen Pool | 59.9 |
| Thymus Pool | 24.0 |
| CNS cancer (glio/astro) U87-MG | 82.4 |
| CNS cancer (glio/astro) U-118-MG | 42.3 |
| CNS cancer (neuro; met) SK-N-AS | 46.7 |
| CNS cancer (astro) SF-539 | 22.1 |
| CNS cancer (astro) SNB-75 | 45.4 |
| CNS cancer (glio) SNB-19 | 31.9 |
| CNS cancer (glio) SF-295 | 60.7 |
| Brain (Amygdala) Pool | 6.9 |
| Brain (cerebellum) | 12.7 |
| Brain (fetal) | 10.0 |
| Brain (Hippocampus) Pool | 10.2 |
| Cerebral Cortex Pool | 8.4 |
| Brain (Substantia nigra) Pool | 7.0 |
| Brain (Thalamus) Pool | 7.6 |
| Brain (whole) | 4.5 |
| Spinal Cord Pool | 22.8 |
| Adrenal Gland | 19.9 |
| Pituitary gland Pool | 5.9 |
| Salivary Gland | 7.3 |
| Thyroid (female) | 30.6 |
| Pancreatic ca. CAPAN2 | 25.7 |
| Pancreas Pool | 29.7 |

General_screening_panel_v1.6 Summary: Ag7050 Highest expression of this gene is seen in adipose (CT=25). This gene is ubiquitously expressed in this panel, with high to moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

B. CG134430-01: RIKEN cDNA 2310034L04 Like Gene.

Expression of gene CG134430-01 was assessed using the primer-probe set Ag7372, described in Table BA. Results of the RTQ-PCR runs are shown in Table BB.

TABLE BA
Probe Name Ag7372

| | | | Start | |
|---|---|---|---|---|
| Primers | Sequences | Length | Position | SEQ ID No |
| Forward | 5′- | 28 | 789 | 258 |
| | catttgaagtggtgtctacacttataaa | | | |
| | -3′ | | | |
| Probe | TET-5′- | 26 | 818 | 259 |
| | agtcctgtccctctggtgcttctcac- | | | |
| | 3′-TAMRA | | | |
| Reverse | 5′-ggcatagatatttcctgattacttcata | 29 | 860 | 260 |
| | t-3′ | | | |

TABLE BB
Panel 4.1D

| | Rel. Exp. (%) |
|---|---|
| | Ag7372, Run |
| Tissue Name | 305065597 |
| Secondary Th1 act | 46.7 |
| Secondary Th2 act | 51.1 |
| Secondary Tr1 act | 27.0 |
| Secondary Th1 rest | 6.1 |
| Secondary Th2 rest | 16.8 |
| Secondary Tr1 rest | 13.9 |
| Primary Th1 act | 25.2 |
| Primary Th2 act | 80.7 |
| Primary Tr1 act | 58.2 |
| Primary Th1 rest | 4.4 |
| Primary Th2 rest | 5.4 |
| Primary Tr1 rest | 0.0 |
| CD45RA CD4 lymphocyte act | 100.0 |
| CD45RO CD4 lymphocyte act | 37.9 |
| CD8 lymphocyte act | 18.2 |
| Secondary CD8 lymphocyte rest | 3.5 |
| Secondary CD8 lymphocyte act | 6.9 |
| CD4 lymphocyte none | 13.1 |
| 2ry Th1/Th2/Tr1_anti-CD95 CH11 | 21.2 |
| LAK cells rest | 5.1 |
| LAK cells IL-2 | 41.8 |
| LAK cells IL-2 + IL-12 | 1.3 |
| LAK cells IL-2 + IFN gamma | 4.0 |
| LAK cells IL-2 + IL-18 | 3.4 |
| LAK cells PMA/ionomycin | 36.3 |
| NK Cells IL-2 rest | 82.4 |
| Two Way MLR 3 day | 14.0 |
| Two Way MLR 5 day | 0.0 |
| Two Way MLR 7 day | 9.1 |
| PBMC rest | 8.3 |
| PBMC PWM | 8.2 |
| PBMC PHA-L | 6.6 |
| Ramos (B cell) none | 9.1 |
| Ramos (B cell) ionomycin | 30.1 |
| B lymphocytes PWM | 9.5 |
| B lymphocytes CD40L and IL-4 | 41.2 |
| EOL-1 dbcAMP | 29.7 |
| EOL-1 dbcAMP PMA/ionomycin | 22.8 |
| Dendritic cells none | 13.8 |
| Dendritic cells LPS | 9.2 |
| Dendritic cells anti-CD40 | 9.8 |
| Monocytes rest | 13.1 |
| Monocytes LPS | 16.2 |
| Macrophages rest | 4.9 |
| Macrophages LPS | 9.1 |
| HUVEC none | 37.1 |
| HUVEC starved | 10.8 |
| HUVEC IL-1beta | 19.1 |
| HUVEC IFN gamma | 17.9 |
| HUVEC TNF alpha + IFN gamma | 3.2 |
| HUVEC TNF alpha + IL4 | 3.9 |
| HUVEC IL-11 | 8.5 |
| Lung Microvascular EC none | 36.9 |
| Lung Microvascular EC TNFalpha + IL-1beta | 8.1 |
| Microvascular Dermal EC none | 3.6 |
| Microsvasular Dermal EC TNFalpha + IL-1beta | 4.0 |
| Bronchial epithelium TNFalpha + IL1beta | 1.4 |
| Small airway epithelium none | 4.6 |
| Small airway epithelium TNFalpha + IL-1beta | 31.6 |
| Coronery artery SMC rest | 6.0 |
| Coronery artery SMC TNFalpha + IL-1beta | 10.4 |
| Astrocytes rest | 2.2 |
| Astrocytes TNFalpha + IL-1beta | 1.7 |
| KU-812 (Basophil) rest | 8.1 |
| KU-812 (Basophil) PMA/ionomycin | 11.3 |
| CCD1106 (Keratinocytes) none | 18.3 |
| CCD1106 (Keratinocytes) TNFalpha + IL-1beta | 4.6 |
| Liver cirrhosis | 3.9 |
| NCI-H292 none | 8.4 |
| NCI-H292 IL-4 | 6.4 |
| NCI-H292 IL-9 | 9.0 |
| NCI-H292 IL-13 | 7.5 |
| NCI-H292 IFN gamma | 2.1 |
| HPAEC none | 7.4 |
| HPAEC TNF alpha + IL-1 beta | 32.1 |
| Lung fibroblast none | 11.9 |
| Lung fibroblast TNF alpha + IL-1 beta | 0.0 |
| Lung fibroblast IL-4 | 9.5 |
| Lung fibroblast IL-9 | 6.4 |
| Lung fibroblast IL-13 | 10.6 |
| Lung fibroblast IFN gamma | 9.1 |
| Dermal fibroblast CCD1070 rest | 25.7 |
| Dermal fibroblast CCD1070 TNF alpha | 47.3 |
| Dermal fibroblast CCD1070 IL-1 beta | 22.2 |
| Dermal fibroblast IFN gamma | 8.9 |
| Dermal fibroblast IL-4 | 11.8 |
| Dermal Fibroblasts rest | 15.0 |
| Neutrophils TNFa + LPS | 31.2 |
| Neutrophils rest | 98.6 |
| Colon | 5.4 |
| Lung | 4.5 |
| Thymus | 16.2 |
| Kidney | 130.8 |

Panel 4.1D Summary: Ag7372 This gene is widely expressed at low levels in many samples on this panel. Highest expression of this gene is seen in CD45RA CD4 cells, naive T cells that have been activated with CD3 and CD28 (CT=32.6). Significant expression is also seen in both acutely and chronically activated T cells, resting neutrophils and NK cells. Based on the widespread expression of this gene in cells of significance to the autoimmune response, modulation of the expression or function of this gene may be useful in the treatment of autoimmune disease, including T cell mediated diseases such as asthma, arthritis, psoriasis, inflammatory bowel disease, and lupus.

C. CG137677-01 and CG137697-01: RIKEN 5730409G15-Like Protein.

Expression of gene CG137677-01 and CG137697-01 was assessed using the primer-probe sets Ag4928 and Ag4927, described in Tables CA and CB. Results of the RTQ-PCR runs are shown in Tables CC, CD and CE.

TABLE CA

Probe Name Ag4928

| | | | Start | |
|---|---|---|---|---|
| Primers | Sequence | Length | Position | SEQ ID No |
| Forward | 5′-tcagatgggaagtgqaagct-3′ | 20 | 935 | 261 |
| Probe | TET-5′-ccagaaactgtttccctacagagagca-3′-TAMRA | 27 | 963 | 262 |
| Reverse | 5′-aggttcagcattgccatct-3′ | 19 | 995 | 263 |

TABLE CB

Probe Name Ag4927

| | | | | |
|---|---|---|---|---|
| Primers | Sequences | Length | Start Position | SEQ ID No |

| | | | | |
|---|---|---|---|---|
| Forward | 5′-ccccaggcatacatcttca-3′ | 19 | 571 | 264 |
| Probe | TET-5′-actgtcacagccgggtactcgag-3′-TAMRA | 23 | 593 | 265 |
| Reverse | 5′-gaggccattgagaaggacat-3′ | 20 | 629 | 266 |

TABLE CC
CNS_neurodegeneration_v1.0

| | Rel. Exp. (%) | Rel. Exp. (%) |
|---|---|---|
| | Ag4927, Run | Ag4928, Run |
| Tissue Name | 224735008 | 224735009 |
| AD 1 Hippo | 4.7 | 14.2 |
| AD 2 Hippo | 42.3 | 66.9 |
| AD 3 Hippo | 4.9 | 7.9 |
| AD 4 Hippo | 9.8 | 12.6 |
| AD 5 hippo | 65.5 | 83.5 |
| AD 6 Hippo | 40.6 | 82.9 |
| Control 2 Hippo | 23.5 | 25.7 |
| Control 4 Hippo | 16.0 | 17.7 |
| Control (Path) 3 Hippo | 0.0 | 15.7 |
| AD 1 Temporal Ctx | 12.7 | 22.5 |
| AD 2 Temporal Ctx | 44.4 | 70.2 |
| AD 3 Temporal Ctx | 5.3 | 2.9 |
| AD 4 Temporal Ctx | 4.1 | 27.0 |
| AD 5 Inf Temporal Ctx | 55.9 | 86.5 |
| AD 5 Sup Temporal Ctx | 33.9 | 70.2 |
| AD 6 Inf Temporal Ctx | 38.4 | 40.3 |
| AD 6 Sup Temporal Ctx | 54.3 | 49.3 |
| Control 1 Temporal Ctx | 8.7 | 12.0 |
| Control 2 Temporal Ctx | 37.4 | 51.8 |
| Control 3 Temporal Ctx | 18.7 | 23.0 |
| Control 4 Temporal Ctx | 17.1 | 20.7 |
| Control (Path) 1 Temporal Ctx | 62.4 | 77.9 |
| Control (Path) 2 Temporal Ctx | 59.5 | 48.6 |
| Control (Path) 3 Temporal Ctx | 8.7 | 11.7 |
| Control (Path) 4 Temporal Ctx | 32.8 | 51.8 |
| AD 1 Occipital Ctx | 14.4 | 9.1 |
| AD 2 Occipital Ctx (Missing) | 0.0 | 0.0 |
| AD 3 Occipital Ctx | 3.9 | 4.9 |
| AD 4 Occipital Ctx | 23.7 | 18.2 |
| AD 5 Occipital Ctx | 0.0 | 66.4 |
| AD 6 Occipital Ctx | 33.0 | 13.4 |
| Control 1 Occipital Ctx | 6.1 | 10.2 |
| Control 2 Occipital Ctx | 61.6 | 47.0 |
| Control 3 Occipital Ctx | 25.3 | 54.3 |
| Control 4 Occipital Ctx | 8.7 | 8.4 |
| Control (Path) 1 Occipital Ctx | 100.0 | 100.0 |
| Control (Path) 2 Occipital Ctx | 4.2 | 18.3 |
| Control (Path) 3 Occipital Ctx | 3.8 | 4.6 |
| Control (Path) 4 Occipital Ctx | 27.5 | 31.9 |
| Control 1 Parietal Ctx | 8.9 | 19.5 |
| Control 2 Parietal Ctx | 33.2 | 46.7 |
| Control 3 Parietal Ctx | 4.1 | 30.6 |
| Control (Path) 1 Parietal Ctx | 76.3 | 73.2 |
| Control (Path) 2 Parietal Ctx | 31.6 | 29.3 |
| Control (Path) 3 Parietal Ctx | 5.0 | 15.9 |
| Control (Path) 4 Parietal Ctx | 57.0 | 52.9 |

TABLE CD
General screening_panel_v1.5

| | Rel.Exp. (%) | Rel. Exp. (%) |
|---|---|---|
| | Ag4927, Run | Ag4928, Run |
| Tissue Name | 228839257 | 228839262 |
| Adipose | 1.4 | 3.1 |
| Melanoma* Hs688(A).T | 12.9 | 18.4 |
| Melanoma* Hs688(B).T | 12.7 | 18.4 |
| Melanoma* M14 | 33.4 | 34.4 |
| Melanoma* LOXIMVI | 34.9 | 25.2 |
| Melanoma* SK-MEL-5 | 37.1 | 68.8 |
| Squamous cell carcinoma SCC-4 | 21.8 | 21.5 |
| Testis Pool | 9.5 | 6.5 |
| Prostate ca.* (bone met) PC-3 | 11.7 | 26.8 |
| Prostate Pool | 4.4 | 4.8 |
| Placenta | 3.2 | 3.8 |
| Uterus Pool | 2.0 | 4.2 |
| Ovarian ca. OVCAR-3 | 25.7 | 34.9 |
| Ovarian ca. SK-OV-3 | 20.6 | 19.5 |
| Ovarian ca. OVCAR-4 | 8.1 | 9.9 |
| Ovarian ca. OVCAR-5 | 44.8 | 44.8 |
| Ovarian ca. IGROV-1 | 8.8 | 27.0 |
| Ovarian ca. OVCAR-8 | 16.0 | 10.2 |
| Ovary | 7.5 | 8.0 |
| Breast ca. MCF-7 | 24.7 | 28.9 |
| Breast ca. MDA-MB-231 | 14.7 | 20.9 |
| Breast ca. BT 549 | 24.3 | 12.8 |
| Breast ca. T47D | 8.7 | 11.1 |
| Breast ca. MDA-N | 18.0 | 18.6 |
| Breast Pool | 18.6 | 8.7 |
| Trachea | 9.4 | 8.0 |
| Lung | 5.3 | 4.5 |
| Fetal Lung | 17.3 | 13.7 |
| Lung ca. NCI-N417 | 8.2 | 5.7 |
| Lung ca. LX-1 | 70.2 | 77.4 |
| Lung ca. NCI-H146 | 9.7 | 4.3 |
| Lung ca. SHP-77 | 34.2 | 26.6 |
| Lung ca. A549 | 25.5 | 29.7 |
| Lung ca. NCI-H526 | 3.1 | 5.4 |
| Lung ca. NCI-H23 | 43.5 | 100.0 |
| Lung ca. NCI-H460 | 25.2 | 24.1 |
| Lung ca. HOP-62 | 12.7 | 11.8 |
| Lung ca. NCI-H522 | 51.1 | 48.6 |
| Liver | 1.2 | 1.6 |
| Fetal Liver | 9.5 | 9.9 |
| Liver ca. HepG2 | 22.1 | 23.2 |
| Kidney Pool | 25.9 | 13.4 |
| Fetal Kidney | 15.7 | 17.6 |
| Renal ca. 786-0 | 12.6 | 14.9 |
| Renal ca. A498 | 7.9 | 7.0 |
| Renal ca. ACHN | 27.2 | 23.8 |
| Renal ca. UO-31 | 21.5 | 20.2 |
| Renal ca. TK-10 | 35.1 | 41.8 |
| Bladder | 13.2 | 10.4 |
| Gastric ca. (liver met.) NCI-N87 | 82.4 | 98.6 |
| Gastric ca. KATO III | 100.0 | 68.3 |
| Colon ca. SW-948 | 15.8 | 10.2 |
| Colon ca. SW480 | 43.8 | 55.5 |
| Colon ca.* (SW480 met) SW620 | 41.8 | 44.4 |
| Colon ca. HT29 | 22.8 | 15.8 |
| Colon ca. HCT-116 | 54.0 | 47.0 |
| Colon ca. CaCo-2 | 31.0 | 47.3 |
| Colon cancer tissue | 9.4 | 7.0 |
| Colon ca. SW1116 | 8.4 | 6.1 |
| Colon ca. Colo-205 | 5.9 | 7.3 |
| Colon ca. SW-48 | 9.2 | 7.5 |
| Colon Pool | 17.3 | 8.5 |
| Small Intestine Pool | 17.7 | 10.8 |
| Stomach Pool | 11.8 | 3.7 |
| Bone Marrow Pool | 5.2 | 3.2 |
| Fetal Heart | 3.1 | 4.1 |
| Heart Pool | 4.6 | 3.7 |
| Lymph Node Pool | 21.2 | 15.5 |
| Fetal Skeletal Muscle | 5.8 | 5.0 |
| Skeletal Muscle Pool | 5.3 | 4.4 |
| Spleen Pool | 3.8 | 2.7 |
| Thymus Pool | 15.9 | 9.5 |
| CNS cancer (glio/astro) U87-MG | 35.1 | 49.3 |
| CNS cancer (glio/astro) U-118-MG | 40.9 | 40.6 |
| CNS cancer (neuro; met) SK-N-AS | 16.7 | 22.4 |
| CNS cancer (astro) SF-539 | 10.7 | 7.1 |
| CNS cancer (astro) SNB-75 | 27.4 | 17.9 |
| CNS cancer (glio) SNB-19 | 12.0 | 15.7 |
| CNS cancer (glio) SF-295 | 38.2 | 43.2 |
| Brain (Amygdala) Pool | 8.2 | 3.4 |
| Brain (cerebellum) | 22.5 | 16.2 |
| Brain (fetal) | 25.5 | 7.9 |
| Brain (Hippocampus) Pool | 7.7 | 4.4 |
| Cerebral Cortex Pool | 10.5 | 5.0 |
| Brain (Substantia nigra) Pool | 9.3 | 4.4 |
| Brain (Thalamus) Pool | 15.0 | 6.7 |
| Brain (whole) | 11.2 | 5.1 |
| Spinal Cord Pool | 11.0 | 5.1 |
| Adrenal Gland | 10.7 | 8.9 |
| Pituitary gland Pool | 6.7 | 9.6 |
| Salivary Gland | 3.7 | 4.7 |
| Thyroid (female) | 3.4 | 4.8 |
| Pancreatic ca. CAPAN2 | 37.4 | 31.9 |
| Pancreas Pool | 23.0 | 13.3 |

TABLE CE
Panel 4.1D

| | Rel. | Rel. |
|---|---|---|
| | Exp. (%) | Exp. (%) |
| | Ag4927, | Ag4928, |
| | Run | Run |
| Tissue Name | 223598856 | 223597247 |
| Secondary Th1 act | 25.2 | 13.8 |
| Secondary Th2 act | 23.5 | 8.7 |
| Secondary Tr1 act | 12.5 | 8.9 |
| Secondary Th1 rest | 6.6 | 4.2 |
| Secondary Th2 rest | 6.4 | 4.2 |
| Secondary Tr1 rest | 6.3 | 2.1 |
| Primary Th1 act | 31.0 | 18.2 |
| Primary Th2 act | 22.8 | 13.0 |
| Primary Tr1 act | 29.9 | 15.9 |
| Primary Th1 rest | 5.7 | 1.6 |
| Primary Th2 rest | 3.8 | 2.5 |
| Primary Tr1 rest | 7.9 | 6.3 |
| CD45RA CD4 lymphocyte act | 10.5 | 10.2 |
| CD45RO CD4 lymphocyte act | 0.0 | 17.8 |
| CD8 lymphocyte act | 28.5 | 15.1 |
| Secondary CD8 lymphocyte rest | 10.6 | 16.2 |
| Secondary CD8 lymphocyte act | 10.2 | 2.6 |
| CD4 lymphocyte none | 4.2 | 2.8 |
| 2ry Th1/Th2/Tr1_anti-CD95 CH11 | 5.7 | 4.7 |
| LAK cells rest | 11.1 | 8.3 |
| LAK cells IL-2 | 15.9 | 11.8 |
| LAK cells IL-2 + IL-12 | 14.1 | 11.2 |
| LAK cells IL-2 + IFN gamma | 18.4 | 11.9 |
| LAK cells IL-2 + IL-18 | 23.0 | 8.4 |
| LAK cells PMA/ionomycin | 5.5 | 0.8 |
| NK Cells IL-2 rest | 11.7 | 6.1 |
| Two Way MLR 3 day | 13.5 | 7.4 |
| Two Way MLR 5 day | 11.3 | 6.0 |
| Two Way MLR 7 day | 13.3 | 7.9 |
| PBMC rest | 2.8 | 1.0 |
| PBMC PWM | 22.5 | 10.3 |
| PBMC PHA-L | 21.2 | 9.0 |
| Ramos (B cell) none | 54.7 | 23.8 |
| Ramos (B cell) ionomycin | 53.2 | 25.9 |
| B lymphocytes PWM | 24.1 | 17.4 |
| B lymphocytes CD40L and IL-4 | 20.4 | 4.0 |
| EOL-1 dbcAMP | 21.8 | 2.9 |
| EOL-1 dbcAMP PMA/ionomycin | 10.2 | 0.0 |
| Dendritic cells none | 7.5 | 4.0 |
| Dendritic cells LPS | 6.3 | 0.8 |
| Dendritic cells anti-CD40 | 7.1 | 5.2 |
| Monocytes rest | 3.8 | 4.2 |
| Monocytes LPS | 4.7 | 0.1 |
| Macrophages rest | 11.7 | 9.9 |
| Macrophages LPS | 2.3 | 1.3 |
| HUVEC none | 13.5 | 5.5 |
| HUVEC starved | 11.5 | 8.2 |
| HUVEC IL-1beta | 15.8 | 9.7 |
| HUVEC IFN gamma | 11.2 | 6.5 |
| HUVEC TNF alpha + IFN gamma | 16.4 | 6.7 |
| HUVEC TNF alpha + IL4 | 20.9 | 10.8 |
| HUVEC IL-11 | 10.4 | 4.3 |
| Lung Microvascular EC none | 29.1 | 12.9 |
| Lung Microvascular EC | 27.0 | 13.0 |
| TNFalpha + IL-1beta | | |
| Microvascular Dermal EC none | 11.2 | 2.5 |
| Microsvasular Dermal EC | 13.8 | 6.9 |
| TNFalpha + IL-1beta | | |
| Bronchial epithelium | 10.1 | 6.7 |
| TNFalpha + IL1beta | | |
| Small airway epithelium none | 7.3 | 2.5 |
| Small airway epithelium | 9.9 | 4.8 |
| TNFalpha + IL-1beta | | |
| Coronery artery SMC rest | 6.7 | 4.6 |
| Coronery artery SMC | 4.9 | 3.5 |
| TNFalpha + IL-1beta | | |
| Astrocytes rest | 11.6 | 5.2 |
| Astrocytes TNFalpha + IL-1beta | 6.1 | 5.3 |
| KU-812 (Basophil) rest | 15.7 | 11.2 |
| KU-812 (Basophil) PMA/ionomycin | 18.9 | 16.0 |
| CCD1106 (Keratinocytes) none | 32.8 | 13.4 |
| CCD1106 (Keratinocytes) | 17.2 | 2.1 |
| TNFalpha + IL-1beta | | |
| Liver cirrhosis | 3.7 | 2.7 |
| NCI-H292 none | 10.4 | 14.6 |
| NCI-H292 IL-4 | 14.9 | 19.6 |
| NCI-H292 IL-9 | 18.2 | 27.2 |
| NCI-H292 IL-13 | 15.6 | 16.4 |
| NCI-H292 IFN gamma | 26.2 | 17.0 |
| HPAEC none | 12.1 | 8.0 |
| HPAEC TNF alpha + IL-1 beta | 19.6 | 14.1 |
| Lung fibroblast none | 36.9 | 24.3 |
| Lung fibroblast | 20.9 | 11.8 |
| TNF alpha + IL-1 beta | | |
| Lung fibroblast IL-4 | 37.4 | 17.6 |
| Lung fibroblast IL-9 | 86.5 | 39.8 |
| Lung fibroblast IL-13 | 43.5 | 20.4 |
| Lung fibroblast IFN gamma | 49.0 | 20.7 |
| Dermal fibroblast CCD1070 rest | 46.3 | 15.2 |
| Dermal fibroblast CCD1070 TNF alpha | 23.7 | 5.9 |
| Dermal fibroblast CCD1070 IL-1 beta | 15.1 | 6.0 |
| Dermal fibroblast IFN gamma | 10.2 | 6.4 |
| Dermal fibroblast IL-4 | 31.4 | 10.7 |
| Dermal Fibroblasts rest | 14.6 | 8.1 |
| Neutrophils TNFa + LPS | 10.4 | 1.5 |
| Neutrophils rest | 11.5 | 1.7 |
| Colon | 3.5 | 2.5 |
| Lung | 9.9 | 3.7 |
| Thymus | 11.2 | 18.8 |
| Kidney | 100.0 | 100.0 |

CNS_neurodegeneration_v1.0 Summary: Ag4927/Ag4928 These results confirm the expression of this gene at low levels in the brains of an independent group of individuals. However, no differential expression of this gene was detected between Alzheimer's diseased postmortem brains and those of non-demented controls in this experiment. See Panel 1.5 for a discussion of this gene in treatment of central nervous system disorders.

General_screening_panel-v1.5 Summary: Ag4927/Ag4928 Two experiments with two different probe and primer sets produce results that are in excellent agreement. Highest expression of this gene is detected in a lung cancer and a gastric cancer cell line (CTs=25-26). Moderate levels of expression of this gene is also seen in cluster of cancer cell lines derived from gastric, colon, lung, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of gastric, colon, lung, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 4.1D Summary: Ag4927/Ag4928 Highest expression of this gene is detected in kidney (CTs=28-29.5). This gene is expressed at moderate to low levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General screening panel v1.5 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

D. CG137717-01: FLJ37712 fis Protein-Like Protein.

Expression of gene CG137717-01 was assessed using the primer-probe set Ag4929, described in Table DA. Results of the RTQ-PCR runs are shown in Tables DB, DC, DD and DE.

TABLE DA

Probe Name Ag4929

| | | | Start | |
|---|---|---|---|---|
| Primers | Sequences | Length | Position | SEQ ID No |
| Forward | 5′-ctcttcatcacctgcattccta-3′ | 22 | 1003 | 267 |
| Probe | TET 5′-tcctctactttaccaaagtggaatactgga-3′-TAMRA | 30 | 1028 | 268 |
| Reverse | 5′-ccatggaatgtcacaaaagag-3′ | 22 | 1059 | 269 |

TABLE DB
A1_comprehensive panel_v1.0

| | Rel. Exp. | | Rel. Exp. |
|---|---|---|---|
| | (%) Ag4929, | | (%) Ag4929, |
| | Run | | Run |
| Tissue Name | 305464508 | Tissue Name | 305464508 |
| 110967 COPD-F | 57.0 | 112427 Match Control | 35.6 |
| | | Psoriasis-F | |
| 110980 COPD-F | 2.7 | 112418 Psoriasis-M | 71.2 |
| 110968 COPD-M | 73.7 | 112723 Match Control | 14.8 |
| | | Psoriasis-M | |
| 110977 COPD-M | 14.4 | 112419 Psoriasis-M | 82.4 |
| 110989 | 46.3 | 112424 Match Control | 4.5 |
| Emphysema-F | | Psoriasis-M | |
| 1110992 | 3.7 | 112420 Psoriasis-M | 34.4 |
| Emphysema-F | | | |
| 110993 | 28.9 | 112425 Match Control | 34.2 |
| Emphysema-F | | Psoriais-M | |
| 110994 | 7.9 | 104689 (MF) OA Bone- | 23.3 |
| Emphysema-F | | Backus | |
| 110995 | 8.1 | 104690 (MF) Adj | 9.0 |
| Emphysema-F | | “Normal” Bone-Backus | |
| 110996 | 1.5 | 104691 (MF) OA | 6.0 |
| Emphysema-F | | Synovium-Backus | |
| 110997 | 1.4 | 104692 (BA) OA | 0.0 |
| Asthma-M | | Cartilage-Backus | |
| 111001 Asthma-F | 4.2 | 104694 (BA) OA Bone- | 5.9 |
| | | Backus | |
| 111002 Asthma-F | 3.9 | 104695 (BA) Adj | 3.9 |
| | | “Normal” Bone-Backus | |
| 111003 Atopic | 11.0 | 104696 (BA) OA | 4.1 |
| Asthma-F | | Synovium-Backus | |
| 111004 Atopic | 13.0 | 104700 (SS) OA Bone- | 16.3 |
| Asthma-F | | Backus | |
| 111005 Atopic | 7.5 | 104701 (SS) Adj | 8.7 |
| Asthma-F | | “Normal” Bone-Backus | |
| 111006 Atopic | 0.5 | 104702 (SS) OA | 7.7 |
| Asthma-F | | Synovium-Backus | |
| 111417 | 4.6 | 117093 OA Cartilage | 15.4 |
| Allergy-M | | Rep7 | |
| 112347 | 0.4 | 112672 OA Bone5 | 30.6 |
| Allergy-M | | | |
| 112349 Normal | 0.3 | 112673 OA Synovium5 | 11.6 |
| Lung-F | | | |
| 112357 Normal | 4.2 | 112674 OA Synovial | 23.3 |
| Lung-F | | Fluid cells5 | |
| 112354 Normal | 5.4 | 117100 OA Cartilage | 6.6 |
| Lung-M | | Rep14 | |
| 112374 Crohns-F | 34.9 | 112756 OA Bone9 | 6.9 |
| 112389 Match | 53.6 | 112757 OA Synovium9 | 14.4 |
| Control Crohns-F | | | |
| 112375 Crohns-F | 33.4 | 112758 OA Synovial | 7.8 |
| | | Fluid Cells9 | |
| 112732 Match | 40.1 | 117125 RA Cartilage | 100.0 |
| Control Crohns-F | | Rep2 | |
| 112725 Crohns-M | 8.9 | 113492 Bone2 RA | 10.7 |
| 112387 Match | 26.6 | 113493 Synovium2 RA | 9.1 |
| Control | | | |
| Crohns-M | | | |
| 112378 Crohns-M | 1.2 | 113494 Syn Fluid Cells | 7.8 |
| | | RA | |
| 112390 Match | 36.1 | 113499 Cartilage4 RA | 15.8 |
| Control | | | |
| Crohns-M | | | |
| 112726 Crohns-M | 17.7 | 113500 Bone4 RA | 24.7 |
| 112731 Match | 43.8 | 1113501 Synovium4 RA | 7.0 |
| Control | | | |
| Crohns-M | | | |
| 112380 Ulcer | 16.6 | 113502 Syn Fluid Cells4 | 13.3 |
| Col-F | | RA | |
| 112734 Match | 88.3 | 113495 Cartliage3 RA | 16.5 |
| Control Ulcer | | | |
| Col-F | | | |
| 112384 Ulcer | 54.7 | 113496 Bone3 RA | 16.7 |
| Col-F | | | |
| 112737 Match | 7.1 | 113497 Synoviym3 RA | 8.9 |
| Control Ulcer | | | |
| Col-F | | | |
| 112386 Ulcer | 6.6 | 113498 Syn Fluid Cells3 | 24.0 |
| Col-F | | RA | |
| 112738 Match | 3.3 | 117106 Normal Cartilage | 5.6 |
| Control Ulcer | | Rep20 | |
| Col-F | | | |
| 112381 | 0.0 | 113663 Bone3 Normal | 0.0 |
| Ulcer Col-M | | | |
| 112735 Match | 5.7 | 113664 Synovium3 | 0.0 |
| Control Ulcer | | Normal | |
| Col-M | | | |
| 112382 | 94.0 | 113665 Syn Fluid Cells3 | 0.5 |
| Ulcer Col-M | | Normal | |
| 112394 Match | 4.1 | 117107 Normal Cartilage | 10.9 |
| Control Ulcer | | Rep22 | |
| Col-M | | | |
| 112383 | 36.6 | 113667 Bone4 Normal | 14.8 |
| Ulcer Col-M | | | |
| 112736 Match | 37.4 | 113668 Synovium4 | 12.0 |
| Control Ulcer | | Normal | |
| Col-M | | | |
| 112423 | 26.6 | 113669 Syn Fluid Cells4 | 17.1 |
| Psoriasis-F | | Normal | |

TABLE DC
CNS_neurodegeneration_v1.0

| | Rel. Exp. (%) |
|---|---|
| | Ag4929, Run |
| Tissue Name | 224735010 |
| AD 1 Hippo | 5.9 |
| AD 2 Hippo | 8.0 |
| AD 3 Hippo | 2.2 |
| AD 4 Hippo | 1.0 |
| AD 5 hippo | 100.0 |
| AD 6 Hippo | 26.6 |
| Control 2 Hippo | 8.8 |
| Control 4 Hippo | 1.2 |
| Control (Path) 3 Hippo | 1.0 |
| AD 1 Temporal Ctx | 6.3 |
| AD 2 Temporal Ctx | 21.2 |
| AD 3 Temporal Ctx | 1.6 |
| AD 4 Temporal Ctx | 9.5 |
| AD 5 Inf Temporal Ctx | 90.1 |
| AD 5 SupTemporal Ctx | 27.7 |
| AD 6 Inf Temporal Ctx | 43.5 |
| AD 6 Sup Temporal Ctx | 41.8 |
| Control 1 Temporal Ctx | 1.5 |
| Control 2 Temporal Ctx | 24.0 |
| Control 3 Temporal Ctx | 10.4 |
| Control 4 Temporal Ctx | 2.3 |
| Control (Path) 1 Temporal Ctx | 39.8 |
| Control (Path) 2 Temporal Ctx | 27.7 |
| Control (Path) 3 Temporal Ctx | 1.1 |
| Control (Path) 4 Temporal Ctx | 25.7 |
| AD 1 Occipital Ctx | 10.8 |
| AD 2 Occipital Ctx (Missing) | 0.0 |
| AD 3 Occipital Ctx | 2.6 |
| AD 4 Occipital Ctx | 10.7 |
| AD 5 Occipital Ctx | 17.6 |
| AD 6 Occipital Ctx | 18.7 |
| Control 1 Occipital Ctx | 0.3 |
| Control 2 Occipital Ctx | 48.0 |
| Control 3 Occipital Ctx | 14.2 |
| Control 4 Occipital Ctx | 1.0 |
| Control (Path) 1 Occipital Ctx | 59.5 |
| Control (Path) 2 Occipital Ctx | 9.9 |
| Control (Path) 3 Occipital Ctx | 0.4 |
| Control (Path) 4 Occipital Ctx | 17.1 |
| Control 1 Parietal Ctx | 1.9 |
| Control 2 Parietal Ctx | 33.2 |
| Control 3 Parietal Ctx | 17.3 |
| Control (Path) 1 Parietal Ctx | 42.6 |
| Control (Path) 2 Parietal Ctx | 16.3 |
| Control (Path) 3 Parietal Ctx | 0.9 |
| Control (Path) 4 Parietal Ctx | 31.6 |

TABLE DD
General_screening_panel_v1.5

| | Rel. Exp. (%) |
|---|---|
| | Ag4929, Run |
| Tissue Name | 228839297 |
| Adipose | 0.0 |
| Melanoma* Hs688(A).T | 0.0 |
| Melanoma* Hs688(B).T | 0.1 |
| Melanoma* M14 | 40.9 |
| Melanoma* LOXIMVI | 0.0 |
| Melanoma* SK-MEL-5 | 0.0 |
| Squamous cell carcinoma SCC-4 | 17.1 |
| Testis Pool | 1.4 |
| Prostate ca.* (bone met) PC-3 | 40.9 |
| Prostate Pool | 1.0 |
| Placenta | 0.0 |
| Uterus Pool | 0.7 |
| Ovarian ca. OVCAR-3 | 1.7 |
| Ovarian ca. SK-OV-3 | 88.3 |
| Ovarian ca. OVCAR-4 | 0.5 |
| Ovarian ca. OVCAR-5 | 44.1 |
| Ovarian ca. IGROV-1 | 3.4 |
| Ovarian ca. OVCAR-8 | 9.1 |
| Ovary | 3.3 |
| Breast ca. MCF-7 | 0.3 |
| Breast ca. MDA-MB-231 | 67.8 |
| Breast ca. BT 549 | 0.4 |
| Breast ca. T47D | 0.0 |
| Breast ca. MDA-N | 0.2 |
| Breast Pool | 1.6 |
| Trachea | 3.5 |
| Lung | 0.0 |
| Fetal Lung | 2.5 |
| Lung ca. NCI-N417 | 0.0 |
| Lung ca. LX-1 | 0.0 |
| Lung ca. NCI-H146 | 0.0 |
| Lung ca. SHP-77 | 6.1 |
| Lung ca. A549 | 32.8 |
| Lung ca. NCI-H526 | 5.3 |
| Lung ca. NCI-H23 | 1.3 |
| Lung ca. NCI-H460 | 21.2 |
| Lung ca. HOP-62 | 15.0 |
| Lung ca. NCI-H522 | 0.6 |
| Liver | 0.0 |
| Fetal Liver | 2.9 |
| Liver ca. HepG2 | 0.0 |
| Kidney Pool | 8.3 |
| Fetal Kidney | 2.2 |
| Renal ca. 786-0 | 3.3 |
| Renal ca. A498 | 12.0 |
| Renal ca. ACHN | 51.1 |
| Renal ca. UO-31 | 19.8 |
| Renal ca. TK-10 | 65.5 |
| Bladder | 0.1 |
| Gastric ca. (liver met.) NCI-N87 | 3.3 |
| Gastric ca. KATO III | 0.0 |
| Colon ca. SW-948 | 7.2 |
| Colon ca. SW480 | 1.8 |
| Colon ca.* (SW480 met) SW620 | 0.2 |
| Colon ca. HT29 | 0.0 |
| Colon ca. HCT-116 | 46.7 |
| Colon ca. CaCo-2 | 0.0 |
| Colon cancer tissue | 0.2 |
| Colon ca. SW1116 | 0.0 |
| Colon ca. Colo-205 | 0.0 |
| Colon ca. SW-48 | 0.0 |
| Colon Pool | 0.9 |
| Small Intestine Pool | 1.4 |
| Stomach Pool | 1.5 |
| Bone Marrow Pool | 0.0 |
| Fetal Heart | 0.0 |
| Heart Pool | 1.6 |
| Lymph Node Pool | 3.0 |
| Fetal Skeletal Muscle | 0.2 |
| Skeletal Muscle Pool | 0.6 |
| Spleen Pool | 1.1 |
| Thymus Pool | 3.5 |
| CNS cancer (glio/astro) U87-MG | 0.2 |
| CNS cancer (glio/astro) U-118-MG | 1.2 |
| CNS cancer (neuro; met) SK-N-AS | 48.0 |
| CNS cancer (astro) SF-539 | 0.0 |
| CNS cancer (astro) SNB-75 | 2.1 |
| CNS cancer (glio) SNB-19 | 5.6 |
| CNS cancer (glio) SF-295 | 0.4 |
| Brain (Amygdala) Pool | 0.0 |
| Brain (cerebellum) | 14.5 |
| Brain (fetal) | 4.7 |
| Brain (Hippocampus) Pool | 10.1 |
| Cerebral Cortex Pool | 0.5 |
| Brain (Substantia nigra) Pool | 1.7 |
| Brain (Thalamus) Pool | 27.9 |
| Brain (whole) | 39.0 |
| Spinal Cord Pool | 14.7 |
| Adrenal Gland | 1.7 |
| Pituitary gland Pool | 4.0 |
| Salivary Gland | 5.9 |
| Thyroid (female) | 0.9 |
| Pancreatic ca. CAPAN2 | 100.0 |
| Pancreas Pool | 1.6 |

TABLE DE
Panel 4.1D

| | Rel. Exp. (%) |
|---|---|
| | Ag4929, Run |
| Tissue Name | 223597249 |
| Secondary Th1 act | 55.1 |
| Secondary Th2 act | 22.2 |
| Secondary Tr1 act | 34.4 |
| Secondary Th1 rest | 3.6 |
| Secondary Th2 rest | 3.8 |
| Secondary Tr1 rest | 13.8 |
| Primary Th1 act | 60.3 |
| Primary Th2 act | 40.1 |
| Primary Tr1 act | 100.0 |
| Primary Th1 rest | 10.4 |
| Primary Th2 rest | 9.6 |
| Primary Tr1 rest | 42.6 |
| CD45RA CD4 lymphocyte act | 7.2 |
| CD45RO CD4 lymphocyte act | 5.9 |
| CD8 lymphocyte act | 11.3 |
| Secondary CD8 lymphocyte rest | 5.7 |
| Secondary CD8 lymphocyte act | 14.7 |
| CD4 lymphocyte none | 2.0 |
| 2ry Th1/Th2/Tr1_anti-CD95 CH11 | 9.7 |
| LAK cells rest | 4.1 |
| LAK cells IL-2 | 1.0 |
| LAK cells IL-2 + IL-12 | 6.9 |
| LAK cells IL-2 + IFN gamma | 12.7 |
| LAK cells IL-2 + IL-18 | 16.2 |
| LAK cells PMA/ionomycin | 0.3 |
| NK Cells IL-2 rest | 4.1 |
| Two Way MLR 3 day | 3.6 |
| Two Way MLR 5 day | 8.1 |
| Two Way MLR 7 day | 3.4 |
| PBMC rest | 0.7 |
| PBMC PWM | 0.7 |
| PBMC PHA-L | 5.8 |
| Ramos (B cell) none | 0.0 |
| Ramos (B cell) ionomycin | 0.0 |
| B lymphocytes PWM | 16.4 |
| B lymphocytes CD40L and IL-4 | 0.9 |
| EOL-1 dbcAMP | 0.0 |
| EOL-1 dbcAMP PMA/ionomycin | 0.0 |
| Dendritic cells none | 1.4 |
| Dendritic cells LPS | 0.4 |
| Dendritic cells anti-CD40 | 1.0 |
| Monocytes rest | 1.0 |
| Monocytes LPS | 0.2 |
| Macrophages rest | 0.8 |
| Macrophages LPS | 0.2 |
| HUVEC none | 0.0 |
| HUVEC starved | 0.0 |
| HUVEC IL-1beta | 0.0 |
| HUVEC IFN gamma | 0.1 |
| HUVEC TNF alpha + IFN gamma | 0.0 |
| HUVEC TNF alpha + IL4 | 0.0 |
| HUVEC IL-11 | 0.7 |
| Lung Microvascular EC none | 0.4 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 4.5 |
| Small airway epithelium none | 5.4 |
| Small airway epithelium TNFalpha + IL-1beta | 7.2 |
| Coronery artery SMC rest | 0.4 |
| Coronery artery SMC TNFalpha + IL-1beta | 2.2 |
| Astrocytes rest | 3.4 |
| Astrocytes TNFalpha + IL-1beta | 2.6 |
| KU-812 (Basophil) rest | 0.0 |
| KU-812 (Basophil) PMA/ionomycin | 0.2 |
| CCD1106 (Keratinocytes) none | 14.1 |
| CCD1106 (Keratinocytes) TNFalpha + IL-1beta | 6.7 |
| Liver cirrhosis | 0.4 |
| NCI-H292 none | 3.2 |
| NCI-H292 IL-4 | 2.5 |
| NCI-H292 IL-9 | 4.4 |
| NCI-H292 IL-13 | 2.9 |
| NCI-H292 IFN gamma | 1.7 |
| hpaec none | 0.0 |
| HPAEC TNF alpha + IL-1 beta | 0.2 |
| Lung fibroblast none | 0.3 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.2 |
| Dermal fibroblast CCD1070 TNF alpha | 3.5 |
| Dermal fibroblast CCD1070 | 1.0 |
| IL-1 beta | |
| Dermal fibroblast IFN gamma | 0.5 |
| Dermal fibroblast IL-4 | 0.0 |
| Dermal Fibroblasts rest | 0.2 |
| Neutrophils TNFa + LPS | 0.5 |
| Neutrophils rest | 0.9 |
| Colon | 0.3 |
| Lung | 2.0 |
| Thymus | 8.1 |
| Kidney | 42.3 |

AI_comprehensive panel_v1.0 Summary: Ag4929 Highest expression of this gene is detected in RA cartilage (CT=30.6). In addition, moderate levels of expression are seen in samples from Crohn's, ulcerative colitis, psoriasis, and COPD derived tissue. Thus, modulation of the expression or function of this gene may be useful in the treatment of these conditions.

CNS_neurodegeneration_v1.0 Summary: Ag4929 This panel does not show differential expression of this gene in Alzheimer's disease. However, this profile confirms the expression of this gene at moderate levels in the brain. Therefore, therapeutic modulation of the expression or function of this gene may be useful in the treatment of neurological disorders, such as Alzheimer's disease, Parkinson's disease, schizophrenia, multiple sclerosis, stroke and epilepsy.

General_screening_panel_v1.5 Summary: Ag4929 Highest expression of this gene is seen in a pancreatic cancer cell line (CT=28). Expression in this panel appears to be predominantly associated with samples derived from cancer cell lines, including brain, renal, lung, breast, ovarian, prostate and melanoma cancer cell lines. Thus, expression of this gene could be used as a marker of cancer. Furthermore, therapeutic modulation of the expression or function of this gene may be useful in the treatment of cancer.

Panel 4.1D Summary: Ag4929 Expression of this gene is most prominent in T cells including both acutely and chronically activated T cells (CTs=29-30). Therefore, therapeutics designed with the protein encoded by this transcript may help to regulate T cell function and be effective in treating T cell mediated diseases such as asthma, arthritis, psoriasis, and lupus.

E. CG137793-02: High Affinity Immunoglobulin Epsilon Receptor Alpha-Subunit Precursor Protein-Like Protein.

Expression of gene CG137793-02 was assessed using the primer-probe set Ag6866, described in Table EA.

TABLE EA
Probe Name Ag6866

| | | | Start | |
|---|---|---|---|---|
| Primers | Sequences | Length | Position | SEQ ID No |
| Forward | 5′-agaatacaaatgccatggtt-3′ | 20 | 292 | 270 |
| Probe | TET-5′- | 32 | 320 | 271 |
| | tccttataatagatcaccttgtacacatcc | | | |
| | ca-3′-TAMRA | | | |
| Reverse | 5′-ggttctcataccagtacttgaga-3′ | 23 | 361 | 272 |

F. CG137873-02: Human Fibrinogen Alpha Chain Precursor Protein-Likew Protein

Expression of gene CG 137873-02 was assessed using the primer-probe set Ag7411, described in Table FA. Results of the RTQ-PCR runs are shown in Table FB.

TABLE FA

Probe Name Ag7411

| | | | Start | |
|---|---|---|---|---|
| Primers | Sequences | Length | Position | SEQ ID No |
| Forward | 5′-acccagactggggctca-3′ | 17 | 1196 | 273 |
| Probe | TET-5′-atctggcatcttcacaaatacaaagg-3′-TAMRA | 26 | 1215 | 274 |
| Reverse | 5′-atttaccacgggaagggaa-3′ | 19 | 1273 | 275 |

TABLE FB
Panel 4.1D

| | Rel. Exp. (%) |
|---|---|
| | Ag7411, Run |
| Tissue Name | 305065220 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| lak cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 0.0 |
| Thymus | 0.0 |
| Kidney | 0.0 |

Panel 4.1D Summary: Ag7411 Significant expression of this gene is detected in a liver cirrhosis sample (CT=33.8). Furthermore, expression of this gene is not detected in normal liver on Panel 1.6, suggesting that its expression is unique to liver cirrhosis. Therefore, therapeutic modulation of the expression or function of this gene may be used to diagnose this condition or to reduce or inhibit fibrosis that occurs in liver cirrhosis.

G. CG137873-03 (205101513edited2): Fibrinogen Alpha Chain Precursor Protein-Like Protein.

Expression of gene CG137873-03 (205101513edited2) was assessed using the primer-probe set Ag7412, described in Table GA. Results of the RTQ-PCR runs are shown in Tables GB and GC.

TABLE GA

Probe Name Ag7412

| | | | | |
|---|---|---|---|---|
| | | | Start | |
| Primers | Sequences | Length | Position | SEQ ID No |

| | | | | |
|---|---|---|---|---|
| Forward | 5′-ggaagctggaagctggaagta-3′ | 21 | 970 | 276 |
| Probe | TET-5′-ccaaaaccctgggagccctagacctg-3′-TAMRA | 26 | 998 | 277 |
| Reverse | 5′-ctgccaggattccaggtt-3′ | 18 | 1034 | 278 |

TABLE GB
General_screening_panel_v1.6

| | Rel. Exp. (%) |
|---|---|
| | Ag7412, Run |
| Tissue Name | 306067375 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.5 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 4.6 |
| Lung ca. NCI-H526 | 0.0 |
| Lung ca. NC1-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 100.0 |
| Liver ca. HepG2 | 6.7 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 3.3 |
| Bladder | 1.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 6.4 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.1 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met)SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 4.6 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

TABLE GC
Panel 4.1D

| | Rel. Exp. (%) |
|---|---|
| | Ag7412, Run |
| Tissue Name | 305065272 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 0.0 |
| Thymus | 0.0 |
| Kidney | 0.0 |

General_screening_panel_v1.6 Summary: Ag7412 Highest expression of this gene is seen in fetal liver (CT=27). Thus, expression of this gene could be used to differentiate between fetal and adult liver (CT=40). Furthermore, therapeutic modulation of the expression or function of this gene may be useful in the treatment of liver disorders.

Panel 4.1D Summary: Ag7412 Significant expression of this gene is detected in a liver cirrhosis sample (CT=28.3). Therefore, therapeutic modulation of the expression or functoin of this gene may be used to diagnose this condition and to reduce or inhibit fibrosis that occurs in liver cirrhosis.

H. CG137882-02: Membrane Protein FLJ212269-Like Protein.

Expression of gene CG 137882-02 was assessed using the primer-probe set Ag7046, described in Table HA.

TABLE HA

Probe Name Ag7046

| | | | Start | |
|---|---|---|---|---|
| Primers | Sequences | Length | Position | SEQ ID No |
| Forward | 5′-tgtgaacgtcgaagcaacc-3′ | 19 | 391 | 279 |
| Probe | TET-5′-agtctcaccttccagcgacaagcttcc-3′-TAMRA | 27 | 421 | 280 |
| Reverse | 5′-tgggagagatattggaaaggaat-3′ | 23 | 461 | 281 |

General_screening_panel_v1.6 Summary: Ag7046 Expression of this gene is low/undetectable in all samples on this panel (CTs>35).

I. CG137910-01: FLJ21432-Like Protein.

Expression of gene CG137910-01 was assessed using the primer-probe set Ag7448, described in Table IA. Results of the RTQ-PCR runs are shown in Tables IB and IC.

TABLE 1A

Probe Name Ag7448

| | | | Start | |
|---|---|---|---|---|
| Primers | Sequences | Length | Position | SEQ ID No |
| Forward | 5′-aggagccattctctgccttt-3′ | 20 | 315 | 282 |
| Probe | TET-5′-catggctcttccacacagtctctactgcca-3′-TAMRA | 28 | 341 | 283 |
| Reverse | 5′-cagtttagagaagagccgagaga-3′ | 23 | 380 | 284 |

TABLE IB
CNS_neurodegeneration_v1.0

| | Rel. Exp. (%) |
|---|---|
| | Ag7448, Run |
| Tissue Name | 306067416 |
| AD 1 Hippo | 12.5 |
| AD 2 Hippo | 29.1 |
| AD 3 Hippo | 9.9 |
| AD 4 Hippo | 6.4 |
| AD 5 hippo | 48.3 |
| AD 6 Hippo | 41.2 |
| Control 2 Hippo | 16.4 |
| Control 4 Hippo | 10.4 |
| Control (Path) 3 Hippo | 2.9 |
| AD 1 Temporal Ctx | 7.3 |
| AD 2 Temporal Ctx | 27.5 |
| AD 3 Temporal Ctx | 9.5 |
| AD 4 Temporal Ctx | 16.2 |
| AD 5 Inf Temporal Ctx | 100.0 |
| AD 5 SupTemporal Ctx | 52.9 |
| AD 6 Inf Temporal Ctx | 58.2 |
| AD 6 Sup Temporal Ctx | 36.1 |
| Control 1 Temporal Ctx | 5.6 |
| Control 2 Temporal Ctx | 32.1 |
| Control 3 Temporal Ctx | 8.5 |
| Control 4 Temporal Ctx | 7.4 |
| Control (Path) 1 Temporal Ctx | 20.6 |
| Control (Path) 2 Temporal Ctx | 16.6 |
| Control (Path) 3 Temporal Ctx | 5.2 |
| Control (Path) 4 Temporal Ctx | 12.2 |
| AD 1 Occipital Ctx | 14.3 |
| AD 2 Occipital Ctx (Missing) | 0.0 |
| AD 3 Occipital Ctx | 16.3 |
| AD 4 Occipital Ctx | 17.3 |
| AD 5 Occipital Ctx | 11.2 |
| AD 6 Occipital Ctx | 25.5 |
| Control 1 Occipital Ctx | 5.8 |
| Control 2 Occipital Ctx | 30.8 |
| Control 3 Occipital Ctx | 11.6 |
| Control 4 Occipital Ctx | 11.6 |
| Control (Path) 1 Occipital Ctx | 47.0 |
| Control (Path) 2 Occipital Ctx | 5.1 |
| Control (Path) 3 Occipital Ctx | 9.5 |
| Control (Path) 4 Occipital Ctx | 9.6 |
| Control 1 Parietal Ctx | 4.1 |
| Control 2 Parietal Ctx | 37.9 |
| Control 3 Parietal Ctx | 10.2 |
| Control (Path) 1 Parietal Ctx | 27.9 |
| Control (Path) 2 Parietal Ctx | 12.8 |
| Control (Path) 3 Parietal Ctx | 9.8 |
| Control (Path) 4 Parietal Ctx | 17.2 |

TABLE IC
Panel 4.1D

| | Rel. Exp. (%) |
|---|---|
| | Ag7448, Run |
| Tissue Name | 306067435 |
| Secondary Th1 act | 53.2 |
| Secondary Th2 act | 81.2 |
| Secondary Tr1 act | 8.6 |
| Secondary Th1 rest | 5.6 |
| Secondary Th2 rest | 6.7 |
| Secondary Tr1 rest | 3.8 |
| Primary Th1 act | 10.7 |
| Primary Th2 act | 51.8 |
| Primary Tr1 act | 62.9 |
| Primary Th1 rest | 3.7 |
| Primary Th2 rest | 5.0 |
| Primary Tr1 rest | 0.6 |
| CD45RA CD4 lymphocyte act | 45.7 |
| CD45RO CD4 lymphocyte act | 68.3 |
| CD8 lymphocyte act | 10.4 |
| Secondary CD8 lymphocyte rest | 12.3 |
| Secondary CD8 lymphocyte act | 12.0 |
| CD4 lymphocyte none | 1.0 |
| 2ry Th1/Th2/Tr1_anti-CD95 CH11 | 9.8 |
| LAK cells rest | 18.2 |
| LAK cells IL-2 | 18.9 |
| LAK cells IL-2 + IL-12 | 1.3 |
| LAK cells IL-2 + IFN gamma | 9.2 |
| LAK cells IL-2 + IL-18 | 10.4 |
| LAK cells PMA/ionomycin | 77.9 |
| NK Cells IL-2 rest | 82.4 |
| Two Way MLR 3 day | 9.5 |
| Two Way MLR 5 day | 5.4 |
| Two Way MLR 7 day | 10.4 |
| PBMC rest | 3.8 |
| PBMC PWM | 20.9 |
| PBMC PHA-L | 15.6 |
| Ramos (B cell) none | 33.4 |
| Ramos (B cell) ionomycin | 97.3 |
| B lymphocytes PWM | 32.5 |
| B lymphocytes CD40L and IL-4 | 45.4 |
| EOL-1 dbcAMP | 64.2 |
| EOL-1 dbcAMP PMA/ionomycin | 13.1 |
| Dendritic cells none | 10.7 |
| Dendritic cells LPS | 4.9 |
| Dendritic cells anti-CD40 | 18.2 |
| Monocytes rest | 7.4 |
| Monocytes LPS | 79.0 |
| Macrophages rest | 77.8 |
| Macrophages LPS | 13.2 |
| HUVEC none | 27.9 |
| HUVEC starved | 51.1 |
| HUVEC IL-1beta | 55.1 |
| HUVEC IFN gamma | 50.7 |
| HUVEC TNF alpha + IFN gamma | 8.2 |
| HUVEC TNF alpha + IL4 | 21.6 |
| HUVEC IL-11 | 20.3 |
| Lung Microvascular EC none | 84.1 |
| Lung Microvascular EC TNFalpha + IL-1beta | 24.3 |
| Microvascular Dermal EC none | 18.0 |
| Microsvasular Dermal EC TNFalpha + IL-1beta | 11.0 |
| Bronchial epithelium TNFalpha + IL1beta | 26.1 |
| Small airway epithelium none | 29.9 |
| Small airway epithelium TNFalpha + IL-1beta | 58.6 |
| Coronery artery SMC rest | 16.0 |
| Coronery artery SMC TNFalpha + IL-1beta | 23.8 |
| Astrocytes rest | 5.5 |
| Astrocytes TNFalpha + IL-1beta | 4.8 |
| KU-812 (Basophil) rest | 34.2 |
| KU-812 (Basophil) PMA/ionomycin | 79.0 |
| CCD1106 (Keratinocytes) none | 16.8 |
| CCD1106 (Keratinocytes) TNFalpha + IL-1beta | 6.9 |
| Liver cirrhosis | 9.4 |
| NCI-H292 none | 40.3 |
| NCI-H292 IL-4 | 68.8 |
| NCI-H292 IL-9 | 75.3 |
| NCI-H292 IL-13 | 39.5 |
| NCI-H292 IFN gamma | 19.6 |
| HPAEC none | 7.2 |
| HPAEC TNF alpha + IL-1 beta | 58.6 |
| Lung fibroblast none | 36.3 |
| Lung fibroblast TNF alpha + IL-1 beta | 23.7 |
| Lung fibroblast IL-4 | 30.8 |
| Lung fibroblast IL-9 | 55.5 |
| Lung fibroblast IL-13 | 7.6 |
| Lung fibroblast IFN gamma | 51.4 |
| Dermal fibroblast CCD1070 rest | 63.7 |
| Dermal fibroblast CCD1070 TNF alpha | 100.0 |
| Dermal fibroblast CCD1070 IL-1 beta | 40.9 |
| Dermal fibroblast IFN gamma | 24.7 |
| Dermal fibroblast IL-4 | 30.8 |
| Dermal Fibroblasts rest | 23.2 |
| Neutrophils TNFa + LPS | 7.6 |
| Neutrophils rest | 15.4 |
| Colon | 8.1 |
| Lung | 4.8 |
| Thymus | 4.7 |
| Kidney | 18.6 |

CNS_neurodegeneration_v1.0 Summary: Ag7448 This gene appears to be upregulated in the temporal cortex of Alzheimer's disease patients when compared with non-demented controls. Therefore, modulation of the expressoin or function of this gene may slow or stop the progression of Alzheimer's disease.

Panel 4.1D Summary: Ag7448 This gene is ubiquitously expressed in this panel with highest expression in TNF-a treated dermal fibroblasts (CT=29). This gene is also expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues as well as in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

J. CG138013-01: Sialic Acid-Binding Immunoglobulin Like Lectin-9-Like Protein.

Expression of gene CG138013-01 was assessed using the primer-probe set Ag4957, described in Table JA. Results of the RTQ-PCR runs are shown in Tables JB, JC and JD.

TABLE JA

Probe Name Ag4957

| | | | Start | |
|---|---|---|---|---|
| Primers | Sequences | Length | Position | SEQ ID No |
| Forward | 5′-cggggagatacttctttcgtat-3′ | 22 | 422 | 285 |
| Probe | TET-5′-tggaattataaacatcaccggctctctg-3′-TAMRA | 28 | 463 | 286 |
| Reverse | 5′-ggtcaaggctgtcacattca-3′ | 20 | 491 | 287 |

TABLE JB
AI_comprehensive panel_v1.0

| | Rel. Exp. (%) |
|---|---|
| | Ag4957, |
| | Run |
| Tissue Name | 222176655 |
| 110967 COPD-F | 6.1 |
| 110980 COPD-F | 3.2 |
| 110968 COPD-M | 6.4 |
| 110977 COPD-M | 4.0 |
| 110989 Emphysema-F | 4.2 |
| 110992 Emphysema-F | 2.3 |
| 110993 Emphysema-F | 3.8 |
| 110994 Emphysema-F | 1.4 |
| 110995 Emphysema-F | 7.1 |
| 110996 Emphysema-F | 2.7 |
| 110997 Asthma-M | 5.1 |
| 111001 Asthma-F | 6.7 |
| 111002 Asthma-F | 5.6 |
| 111003 Atopic Asthma-F | 1.4 |
| 111004 Atopic Asthma-F | 4.0 |
| 111005 Atopic Asthma-F | 2.0 |
| 111006 Atopic Asthma-F | 0.5 |
| 111417 Allergy-M | 4.0 |
| 112347 Allergy-M | 0.0 |
| 112349 Normal Lung-F | 0.5 |
| 112357 Normal Lung-F | 16.7 |
| 112354 Normal Lung-M | 7.7 |
| 112374 Crohns-F | 7.2 |
| 112389 Match Control Crohns-F | 5.5 |
| 112375 Crohns-F | 1.6 |
| 112732 Match Control Crohns-F | 5.2 |
| 112725 Crohns-M | 1.1 |
| 112387 Match Control Crohns-M | 3.4 |
| 112378 Crohns-M | 0.8 |
| 112390 Match Control Crohns-M | 3.9 |
| 112726 Crohns-M | 2.2 |
| 112731 Match Control Crohns-M | 1.7 |
| 112380 Ulcer Col-F | 1.9 |
| 112734 Match Control Ulcer Col-F | 15.5 |
| 112384 Ulcer Col-F | 6.7 |
| 112737 Match Control Ulcer Col-F | 1.2 |
| 112386 Ulcer Col-F | 1.1 |
| 112738 Match Control Ulcer Col-F | 4.1 |
| 112381 Ulcer Col-M | 0.0 |
| 112735 Match Control Ulcer Col-M | 5.6 |
| 112382 Ulcer Col-M | 5.1 |
| 112394 Match Control Ulcer Col-M | 0.4 |
| 112383 Ulcer Col-M | 15.1 |
| 112736 Match Control Ulcer Col-M | 1.4 |
| 112423 Psoriasis-F | 4.0 |
| 112427 Match Control Psoriasis-F | 5.7 |
| 112418 Psoriasis-M | 4.4 |
| 112723 Match Control Psoriasis-M | 2.6 |
| 112419 Psoriasis-M | 9.0 |
| 112424 Match Control Psoriasis-M | 4.2 |
| 112420 Psoriasis-M | 8.1 |
| 112425 Match Control Psoriasis-M | 3.5 |
| 104689 (MF) OA Bone-Backus | 29.3 |
| 104690 (MF) Adj “Normal” Bone-Backus | 11.1 |
| 104691 (MF) OA Synovium-Backus | 37.9 |
| 104692 (BA) OA Cartilage-Backus | 0.9 |
| 104694 (BA) OA Bone-Backus | 29.5 |
| 104695 (BA) Adj “Normal” Bone-Backus | 10.4 |
| 104696 (BA) OA Synovium-Backus | 100.0 |
| 104700 (SS) OA Bone-Backus | 31.0 |
| 104701 (SS) Adj “Normal” Bone-Backus | 19.2 |
| 104702 (SS) OA Synovium-Backus | 28.3 |
| 117093 OA Cartilage Rep7 | 4.0 |
| 112672 OA Bone5 | 8.8 |
| 112673 OA Synovium5 | 4.5 |
| 112674 OA Synovial Fluid cells5 | 2.7 |
| 117100 OA Cartilage Rep14 | 3.1 |
| 112756 OA Bone9 | 6.0 |
| 112757 OA Synovium9 | 0.7 |
| 112758 OA Synovial Fluid Cells9 | 5.5 |
| 117125 RA Cartilage Rep2 | 5.0 |
| 113492 Bone2 RA | 17.9 |
| 113493 Synovium2 RA | 8.1 |
| 113494 Syn Fluid Cells RA | 14.0 |
| 113499 Cartilage4 RA | 17.4 |
| 113500 Bone4 RA | 16.3 |
| 113501 Synovium4 RA | 12.5 |
| 113502 Syn Fluid Cells4 RA | 10.7 |
| 113495 Cartilage3 RA | 24.7 |
| 113496 Bone3 RA | 23.8 |
| 113497 Synovium3 RA | 12.6 |
| 113498 Syn Fluid Cells3 RA | 26.2 |
| 117106 Normal Cartilage Rep20 | 2.5 |
| 113663 Bone3 Normal | 0.7 |
| 113664 Synovium3 Normal | 0.0 |
| 113665 Syn Fluid Cells3 Normal | 0.0 |
| 117107 Normal Cartilage Rep22 | 1.7 |
| 113667 Bone4 Normal | 1.8 |
| 113668 Synovium4 Normal | 1.5 |
| 113669 Syn Fluid Cells4 Normal | 4.1 |

TABLE JC
Panel 4.1D

| | Rel. Exp. (%) |
|---|---|
| | Ag4957, |
| | Run |
| Tissue Name | 219311035 |
| Secondary Th1 act | 0.0 |
| Secondary Th2 act | 0.1 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.1 |
| Primary Th2 rest | 0.0 |
| Primary Tr1 rest | 0.0 |
| CD45RA CD4 lymphocyte act | 0.0 |
| CD45RO CD4 lymphocyte act | 0.4 |
| CD8 lymphocyte act | 0.3 |
| Secondary CD8 lymphocyte rest | 0.4 |
| Secondary CD8 lymphocyte act | 0.0 |
| CD4 lymphocyte none | 0.5 |
| 2ry Th1/Th2/Tr1_anti-CD95 CH11 | 0.0 |
| LAK cells rest | 17.0 |
| LAK cells IL-2 | 0.2 |
| LAK cells IL-2 + IL-12 | 0.4 |
| LAK cells IL-2 + IFN gamma | 0.5 |
| LAK cells IL-2 + IL-18 | 0.3 |
| LAK cells PMA/ionomycin | 8.5 |
| NK Cells IL-2 rest | 1.7 |
| Two Way MLR 3 day | 10.4 |
| Two Way MLR 5 day | 3.9 |
| Two Way MLR 7 day | 0.8 |
| PBMC rest | 7.5 |
| PBMC PWM | 2.4 |
| PBMC PHA-L | 4.0 |
| Ramos (B cell) none | 0.0 |
| Ramos (B cell) ionomycin | 0.0 |
| B lymphocytes PWM | 0.2 |
| B lymphocytes CD40L and IL-4 | 0.1 |
| EOL-1 dbcAMP | 1.4 |
| EOL-1 dbcAMP PMA/ionomycin | 5.7 |
| Dendritic cells none | 35.6 |
| Dendritic cells LPS | 25.5 |
| Dendritic cells anti-CD40 | 57.4 |
| Monocytes rest | 51.8 |
| Monocytes LPS | 100.0 |
| Macrophages rest | 29.1 |
| Macrophages LPS | 12.2 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.2 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.1 |
| Small airway epithelium TNFalpha + IL-1beta | 0.0 |
| Coronery artery SMC rest | 0.0 |
| Coronery artery SMC TNFalpha + IL-1beta | 0.2 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.4 |
| CCD1106 (Keratinocytes) none | 0.0 |
| CCD1106 (Keratinocytes) TNFalpha + IL-1beta | 0.0 |
| Liver cirrhosis | 1.0 |
| NCI-H292 none | 0.0 |
| NCI-H292 IL-4 | 0.0 |
| NCI-H292 IL-9 | 0.0 |
| NCI-H292 IL-13 | 0.1 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.1 |
| Dermal fibroblast CCD1070 IL-1 beta | 0.0 |
| Dermal fibroblast IFN gamma | 0.0 |
| Dermal fibroblast IL-4 | 0.3 |
| Dermal Fibroblasts rest | 0.0 |
| Neutrophils TNFa + LPS | 3.9 |
| Neutrophils rest | 40.3 |
| Colon | 0.1 |
| Lung | 19.9 |
| Thymus | 0.5 |
| Kidney | 0.1 |

TABLE JD
general oncology screening panel_v_2.4

| | Rel. Exp. (%) |
|---|---|
| | Ag4957, |
| | Run |
| Tissue Name | 260281958 |
| Colon cancer 1 | 20.9 |
| Colon cancer NAT 1 | 14.6 |
| Colon cancer 2 | 34.9 |
| Colon cancer NAT 2 | 5.5 |
| Colon cancer 3 | 22.5 |
| Colon cancer NAT 3 | 4.2 |
| Colon malignant cancer 4 | 44.1 |
| Colon normal adjacent tissue 4 | 5.6 |
| Lung cancer 1 | 33.4 |
| Lung NAT 1 | 12.2 |
| Lung cancer 2 | 22.4 |
| Lung NAT 2 | 4.8 |
| Squamous cell carcinoma 3 | 43.2 |
| Lung NAT 3 | 2.9 |
| metastatic melanoma 1 | 16.6 |
| Melanoma 2 | 3.2 |
| Melanoma 3 | 0.0 |
| metastatic melanoma 4 | 70.7 |
| metastatic melanoma 5 | 100.0 |
| Bladder cancer 1 | 3.5 |
| Bladder cancer NAT 1 | 0.0 |
| Bladder cancer 2 | 4.2 |
| Bladder cancer NAT 2 | 0.0 |
| Bladder cancer NAT 3 | 0.0 |
| Bladder cancer NAT 4 | 0.0 |
| Prostate adenocarcinoma 1 | 13.5 |
| Prostate adenocarcinoma 2 | 1.5 |
| Prostate adenocarcinoma 3 | 3.3 |
| Prostate adenocarcinoma 4 | 17.1 |
| Prostate cancer NAT 5 | 3.0 |
| Prostate adenocarcinoma 6 | 1.4 |
| Prostate adenocarcinoma 7 | 3.0 |
| Prostate adenocarcinoma 8 | 0.0 |
| Prostate adenocarcinoma 9 | 10.4 |
| Prostate cancer NAT 10 | 0.0 |
| Kidney cancer 1 | 73.2 |
| KidneyNAT 1 | 11.1 |
| Kidney cancer 2 | 80.7 |
| Kidney NAT 2 | 4.0 |
| Kidney cancer 3 | 20.0 |
| Kidney NAT 3 | 3.1 |
| Kidney cancer 4 | 23.5 |
| Kidney NAT 4 | 5.0 |

AI_comprehensive panel_v1.0 Summary: Ag4957 Highest expression of this gene is detected in orthoarthritis synovium (CT=31.5). In addition, moderate to low levels of expression of this gene is also seen in samples derived from osteoarthritic (OA) bone and adjacent bone as well as OA and normal bone, and OA synovium. Low level expression is also detected in cartilage, bone, synovium and synovial fluid samples from rheumatoid arthritis patients. This gene codes for a variant of sialic acid-binding immunoglobulin-like lectin-9 (SIGLEC-9) protein. Siglec-9 was found to be expressed at high or intermediate levels by monocytes, neutrophils, and a minor population of CD16(+), CD56(−) cells and at lower levels in B cells, NK cells and minor subsets of CD8(+) T cells and CD4(+) T cells (Zhang et al., 2000, J Biol Chem 275(29):22121-6, PMID: 10801862). Similar pattern of expression of SIGLEC-9 encoded by this gene in monocytes, neutrophils and T cells, is also seen in panel 4.1D. Monocytes and T cells are know to play a role in the pathogenesis of arthritis (VanderBorght et al., 2001, Semin Arthritis Rheum 31(3): 160-75, PMID: 11740797; Jenkins JK et al., 2002, Am J Med Sci 323(4):171-80, PMID: 12003371). Therefore, therapeutic modulation of the SIGLEC-9 protein encoded by this gene may be useful in the treatment of osteoarthritis and rheumatoid arthritis.

Panel 4.1D Summary: Ag4957 Highest expression of this gene is detected in LPS treated monocytes (CT=28.5). In addition, moderate levels of expression of this gene is also seen in resting monocytes, dendritic cell, and macrophages. Thus, therapeutic modalities that block the function of the this gene product may be useful in the reduction or elimination of the symptoms in patients with autoimmune and inflammatory diseases in which monocytes, dendritic cells and macrophages play an important role in antigen presentation and other functions. Furthermore, moderate to low levels of expression of this gene is also seen in eosinophils, PBMC cells, two way MLR, LAK cells, stimulated neutrophils and lung. Therefore, therapeutic modulation of this gene product may be beneficial in the treatment of autoimmune and inflammatory diseases, such as lupus erythematosus, Crohn's disease, ulcerative colitis, multiple sclerosis, chronic obstructive pulmonary disease, asthma, emphysema, or rheumatoid arthritis.

general oncology screening panel_v_{—}2.4 Summary: Ag4957 Highest expression of this gene is detected in metastic melanoma (CT=33.2). Moderate to low levels of expression of this gene is also seen in malignant colon cancer, lung cancer, and kidney cancer. Expression of this gene is higher in cancer as compared to the corresponding adjacent normal tissue. Therefore, expression of this gene may be used as diagnostic marker for detection of these cancers and therapeutic modulation of this gene or its product through the use of small molecule drug or antibodies may be useful in the treatment of these cancers and also their metastasis.

K. CG138074-01: RIKEN 2310012P03-Like Protein.

Expression of gene CG 138074-01 was assessed using the primer-probe set Ag4952, described in Table KA.

TABLE KA

Probe Name Ag4952

| | | | Start | |
|---|---|---|---|---|
| Primers | Sequences | Length | Position | SEQ ID No |
| Forward | 5′-tacaccaccatgctgtccat-3′ | 20 | 574 | 288 |
| Probe | TET-5′-ccatatccattctgccttggacacct-3′-TAMRA | 26 | 609 | 289 |
| Reverse | 5′-actcgtgtcactcatcatgtca-3′ | 22 | 648 | 290 |

L. CG138573-01: Folate Receptor 3-Like Protein.

Expression of gene CG138573-01 was assessed using the primer-probe set Ag4964, described in Table LA.

TABLE LA

Probe Name Ag4964

| | | | Start | |
|---|---|---|---|---|
| Primers | Sequences | Length | Position | SEQ ID No |
| Forward | 5′-ctggatgtatccccactctaca-3′ | 22 | 256 | 291 |
| Probe | TET-5′-ttcagcctgtttcactgtggactgct-3′-TAMRA | 26 | 280 | 292 |
| Reverse | 5′-tagaagcagatagcctggatga-3′ | 22 | 329 | 293 |

General_screening_panel_v1.5 Summary: Ag4964 Expression of this gene is low/undetectable in all samples on this panel (CTs>35).

Panel 4.1D Summary: Ag4964 Expression of this gene is low/undetectable in all samples on this panel (CTs>35).

M. CG138606-01: Brush Border 61.9 Kda Protein Precursor-Like Protein.

Expression of gene CG138606-01 was assessed using the primer-probe set Ag4970, described in Table MA. Results of the RTQ-PCR runs are shown in Tables MB and MC.

TABLE MA

Probe Name Ag4970

| | | | Start | |
|---|---|---|---|---|
| Primers | Sequences | Length | Position | SEQ ID No |
| Forward | 5′-ttatatccctcgggaaattgac-3′ | 22 | 1248 | 294 |
| Probe | TET-5′-aaacacagccatcacctttg-3′-TAMRA | 26 | 1287 | 295 |
| Reverse | 5′-tgtcaatgggaaatggtctaaa-3′ | 22 | 1321 | 296 |

TABLE MB
General_screening_panel_v1.5

| | Rel. Exp. (%) |
|---|---|
| | Ag4970, |
| | Run |
| Tissue Name | 228926385 |
| Adipose | 5.1 |
| Melanoma* Hs688(A).T | 1.4 |
| Melanoma* Hs688(B).T | 3.1 |
| Melanoma* M14 | 0.0 |
| Melanoma* LOXIMVI | 0.0 |
| Melanoma* SK-MEL-5 | 0.0 |
| Squamous cell carcinoma SCC-4 | 0.7 |
| Testis Pool | 21.6 |
| Prostate ca.* (bone met) PC-3 | 0.7 |
| Prostate Pool | 2.7 |
| Placenta | 0.0 |
| Uterus Pool | 3.4 |
| Ovarian ca. OVCAR-3 | 0.0 |
| Ovarian ca. SK-OV-3 | 10.7 |
| Ovarian ca. OVCAR-4 | 0.6 |
| Ovarian ca. OVCAR-5 | 0.0 |
| Ovarian ca. IGROV-1 | 0.5 |
| Ovarian ca. OVCAR-8 | 0.0 |
| Ovary | 5.0 |
| Breast ca. MCF-7 | 0.5 |
| Breast ca. MDA-MB-231 | 0.5 |
| Breast ca. BT 549 | 5.4 |
| Breast ca. T47D | 0.0 |
| Breast ca. MDA-N | 0.0 |
| Breast Pool | 9.4 |
| Trachea | 0.9 |
| Lung | 4.3 |
| Fetal Lung | 5.1 |
| Lung ca. NCI-N417 | 0.0 |
| Lung ca. LX-1 | 0.0 |
| Lung ca. NCI-H146 | 0.0 |
| Lung ca. SHP-77 | 1.1 |
| Lung ca. A549 | 0.0 |
| Lung ca. NCI-H526 | 0.0 |
| Lung ca. NCI-H23 | 1.2 |
| Lung ca. NCI-H460 | 0.8 |
| Lung ca. HOP-62 | 0.9 |
| Lung ca. NCI-H522 | 0.3 |
| Liver | 0.0 |
| Fetal Liver | 3.7 |
| Liver ca. HepG2 | 0.6 |
| Kidney Pool | 21.0 |
| Fetal Kidney | 13.6 |
| Renal ca. 786-0 | 0.0 |
| Renal ca. A498 | 0.0 |
| Renal ca. ACHN | 0.0 |
| Renal ca. UO-31 | 1.1 |
| Renal ca. TK-10 | 0.0 |
| Bladder | 2.9 |
| Gastric ca. (liver met.) NCI-N87 | 0.0 |
| Gastric ca. KATO III | 0.0 |
| Colon ca. SW-948 | 4.1 |
| Colon ca. SW480 | 1.3 |
| Colon ca.* (SW480 met) SW620 | 0.0 |
| Colon ca. HT29 | 0.0 |
| Colon ca. HCT-116 | 1.2 |
| Colon ca. CaCo-2 | 2.6 |
| Colon cancer tissue | 3.5 |
| Colon ca. SW1116 | 0.0 |
| Colon ca. Colo-205 | 0.0 |
| Colon ca. SW-48 | 0.0 |
| Colon Pool | 16.7 |
| Small Intestine Pool | 100.0 |
| Stomach Pool | 4.9 |
| Bone Marrow Pool | 6.0 |
| Fetal Heart | 4.2 |
| Heart Pool | 3.2 |
| Lymph Node Pool | 13.5 |
| Fetal Skeletal Muscle | 1.8 |
| Skeletal Muscle Pool | 5.7 |
| Spleen Pool | 3.3 |
| Thymus Pool | 8.0 |
| CNS cancer (glio/astro) U87-MG | 0.0 |
| CNS cancer (glio/astro) U-118-MG | 2.2 |
| CNS cancer (neuro; met) SK-N-AS | 0.0 |
| CNS cancer (astro) SF-539 | 0.0 |
| CNS cancer (astro) SNB-75 | 4.4 |
| CNS cancer (glio) SNB-19 | 0.5 |
| CNS cancer (glio) SF-295 | 1.7 |
| Brain (Amygdala) Pool | 1.5 |
| Brain (cerebellum) | 1.2 |
| Brain (fetal) | 1.3 |
| Brain (Hippocampus) Pool | 2.0 |
| Cerebral Cortex Pool | 1.9 |
| Brain (Substantia nigra) Pool | 1.1 |
| Brain (Thalamus) Pool | 0.9 |
| Brain (whole) | 0.5 |
| Spinal Cord Pool | 2.1 |
| Adrenal Gland | 0.4 |
| Pituitary gland Pool | 0.0 |
| Salivary Gland | 4.6 |
| Thyroid (female) | 0.0 |
| Pancreatic ca. CAPAN2 | 0.0 |
| Pancreas Pool | 9.5 |

TABLE MC
Panel 4.1D

| | Rel. Exp. (%) |
|---|---|
| | Ag4970, |
| | Run |
| Tissue Name | 223692673 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 1.1 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 1.1 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.8 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.7 |
| HUVEC IL-1beta | 0.0 |
| HUVEC IFN gamma | 0.8 |
| HUVEC TNF alpha + IFN gamma | 0.0 |
| HUVEC TNF alpha + IL4 | 3.0 |
| HUVEC IL-11 | 0.0 |
| Lung Microvascular EC none | 0.0 |
| Lung Microvascular EC TNFalpha + IL-1beta | 0.9 |
| Microvascular Dermal EC none | 0.4 |
| Microsvasular Dermal EC TNFalpha + IL-1beta | 0.0 |
| Bronchial epithelium TNFalpha + IL1beta | 2.6 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.7 |
| KU-812 (Basophil) rest | 3.2 |
| KU-812 (Basophil) PMA/ionomycin | 8.4 |
| CCD1106 (Keratinocytes) none | 1.2 |
| CCD1106 (Keratinocytes) TNFalpha + IL-1beta | 0.0 |
| Liver cirrhosis | 100.0 |
| NCI-H292 none | 0.9 |
| NCI-H292 IL-4 | 0.0 |
| NCI-H292 IL-9 | 0.0 |
| NCI-H292 IL-13 | 1.3 |
| NCI-H292 IFN gamma | 0.0 |
| HPAEC none | 1.8 |
| HPAEC TNF alpha + IL-1 beta | 0.8 |
| Lung fibroblast none | 3.0 |
| Lung fibroblast TNF alpha + IL-1 beta | 1.5 |
| Lung fibroblast IL-4 | 0.9 |
| Lung fibroblast IL-9 | 2.8 |
| Lung fibroblast IL-13 | 1.8 |
| Lung fibroblast IFN gamma | 0.9 |
| Dermal fibroblast CCD1070 rest | 0.0 |
| Dermal fibroblast CCD1070 TNF alpha | 0.0 |
| Dermal fibroblast CCD1070 IL-1 beta | 0.8 |
| Dermal fibroblast IFN gamma | 3.0 |
| Dermal fibroblast IL-4 | 1.7 |
| Dermal Fibroblasts rest | 2.4 |
| Neutrophils TNFa + LPS | 0.0 |
| Neutrophils rest | 0.0 |
| Colon | 15.6 |
| Lung | 0.0 |
| Thymus | 0.9 |
| Kidney | 7.0 |

General_screening_panel_v1.5 Summary: Ag4970 Expression of this gene is almost exclusive to small intestine (CT=31.2). Thus, expression of this gene could be used to differentiate between this sample and other samples on this panel.

Panel 4.1D Summary: Ag4970 Significant expression of this gene is detected in a liver cirrhosis sample (CT=30.2). Furthermore, expression of this gene is not detected in normal liver in Panel 1.3 D, suggesting that its expression is unique to liver cirrhosis. Therefore, therapeutic modulation of the expression or function of this gene may be used to diagnose this condition and to reduce or inhibit fibrosis that occurs in liver cirrhosis.

N. CG138751-01: Camp Inducible 2 Protein-Like-Protein.

Expression of gene CG 138751-01 was assessed using the primer-probe set Ag4971, described in Table NA. Results of the RTQ-PCR runs are shown in Tables NB, NC and ND.

TABLE NA

Probe Name Ag4971

| | | | Start | |
|---|---|---|---|---|
| Primers | Sequence | Length | Position | SEQ ID No |
| Forward | 5′-ggaagcctatcagtatcgtcaa-3′ | 22 | 179 | 297 |
| Probe | TET-5′-cggagcagatcaaacccatcaatgat-3′-TAMRA | 26 | 224 | 298 |
| Reverse | 5′cacatggtgtcattgagactgt-3′ | 22 | 254 | 299 |

TABLE NB
A1_comprehensive panel_v1.0

| | Rel. Exp. (%) |
|---|---|
| | Ag4971, |
| | Run |
| Tissue Name | 296465693 |
| 110967 COPD-F | 0.8 |
| 110980 COPD-F | 0.7 |
| 110968 COPD-M | 1.0 |
| 110977 COPD-M | 1.4 |
| 110989 Emphysema-F | 0.6 |
| 110992 Emphysema-F | 0.4 |
| 110993 Emphysema-F | 0.9 |
| 110994 Emphysema-F | 0.5 |
| 110995 Emphysema-F | 1.6 |
| 110996 Emphysema-F | 0.3 |
| 110997 Asthma-M | 0.2 |
| 111001 Asthma-F | 0.7 |
| 111002 Asthma-F | 1.0 |
| 111003 Atopic Asthma-F | 1.2 |
| 111004 Atopic Asthma-F | 1.3 |
| 111005 Atopic Asthma-F | 0.7 |
| 111006 Atopic Asthma-F | 0.2 |
| 111417 Allergy-M | 0.4 |
| 112347 Allergy-M | 0.0 |
| 112349 Normal Lung-F | 0.0 |
| 112357 Normal Lung-F | 0.4 |
| 112354 Normal Lung-M | 0.0 |
| 112374 Crohns-F | 1.0 |
| 112389 Match Control Crohns-F | 2.6 |
| 112375 Crohns-F | 1.0 |
| 112732 Match Control Crohns-F | 3.2 |
| 112725 Crohns-M | 0.1 |
| 112387 Match Control Crohns-M | 0.6 |
| 112378 Crohns-M | 0.0 |
| 112390 Match Control Crohns-M | 0.0 |
| 112726 Crohns-M | 0.9 |
| 112731 Match Control Crohns-M | 0.0 |
| 112380 Ulcer Col-F | 0.3 |
| 112734 Match Control Ulcer Col-F | 6.1 |
| 112384 Ulcer Col-F | 1.1 |
| 112737 Match Control Ulcer Col-F | 0.2 |
| 112386 Ulcer Col-F | 0.7 |
| 112738 Match Control Ulcer Col-F | 1.1 |
| 112381 Ulcer Col-M | 0.0 |
| 112735 Match Control Ulcer Col-M | 0.1 |
| 112382 Ulcer Col-M | 2.3 |
| 112394 Match Control Ulcer Col-M | 0.3 |
| 112383 Ulcer Col-M | 1.8 |
| 112736 Match Control Ulcer Col-M | 2.7 |
| 112423 Psoriasis-F | 0.4 |
| 112427 Match Control Psoriasis-F | 0.1 |
| 112418 Psoriasis-M | 0.7 |
| 112723 Match Control Psoriasis-M | 0.5 |
| 112419 Psoriasis-M | 0.9 |
| 112424 Match Control Psoriasis-M | 0.2 |
| 112420 Psoriasis-M | 1.6 |
| 112425 Match Control Psoriasis-M | 0.0 |
| 104689 (MF) OA Bone-Backus | 63.3 |
| 104690 (MF) Adj “Normal” Bone-Backus | 10.4 |
| 104691 (MF) OA Synovium-Backus | 39.0 |
| 104692 (BA) OA Cartilage-Backus | 0.0 |
| 104694 (BA) OA Bone-Backus | 100.0 |
| 104695 (BA) Adj “Normal” Bone-Backus | 32.5 |
| 104696 (BA) OA Synovium-Backus | 38.4 |
| 104700 (SS) OA Bone-Backus | 8.9 |
| 104701 (SS) Adj “Normal” Bone-Backus | 20.4 |
| 104702 (SS) OA Synovium-Backus | 17.9 |
| 117093 OA Cartilage Rep7 | 1.0 |
| 112672 OA Bone5 | 0.1 |
| 112673 OA Synovium5 | 0.0 |
| 112674 OA Synovial Fluid cells5 | 0.0 |
| 117100 OA Cartilage Rep14 | 0.6 |
| 112756 OA Bone9 | 0.4 |
| 112757 OA Synovium9 | 0.2 |
| 112758 OA Synovial Fluid Cells9 | 0.4 |
| 117125 RA Cartilage Rep2 | 2.2 |
| 113492 Bone2 RA | 2.2 |
| 113493 Synovium2 RA | 0.3 |
| 113494 Syn Fluid Cells RA | 1.5 |
| 113499 Cartilage4 RA | 1.0 |
| 113500 Bone4 RA | 1.2 |
| 113501 Synovium4 RA | 0.6 |
| 113502 Syn Fluid Cells4 RA | 0.6 |
| 113495 Cartilage3 RA | 1.3 |
| 113496 Bone3 RA | 1.5 |
| 113497 Synovium3 RA | 0.9 |
| 113498 Syn Fluid Cells3 RA | 2.0 |
| 117106 Normal Cartilage Rep20 | 0.5 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.2 |
| 113667 Bone4 Normal | 0.1 |
| 113668 Synovium4 Normal | 0.2 |
| 113669 Syn Fluid Cells4 Normal | 0.2 |

TABLE NC
General_screening_panel_v1.5

| | Rel. Exp. (%) |
|---|---|
| | Ag4971, |
| | Run |
| Tissue Name | 228926585 |
| Adipose | 4.0 |
| Melanoma* Hs688(A).T | 0.0 |
| Melanoma* Hs688(B).T | 0.4 |
| Melanoma* M14 | 19.2 |
| Melanoma* LOXIMVI | 0.1 |
| Melanoma* SK-MEL-5 | 67.4 |
| Squamous cell carcinoma SCC-4 | 24.8 |
| Testis Pool | 0.7 |
| Prostate ca.* (bone met) PC-3 | 6.2 |
| Prostate Pool | 0.9 |
| Placenta | 4.2 |
| Uterus Pool | 0.6 |
| Ovarian ca. OVCAR-3 | 0.0 |
| Ovarian ca. SK-OV-3 | 1.2 |
| Ovarian ca. OVCAR-4 | 0.0 |
| Ovarian ca. OVCAR-5 | 3.3 |
| Ovarian ca. IGROV-1 | 0.0 |
| Ovarian ca. OVCAR-8 | 0.1 |
| Ovary | 0.8 |
| Breast ca. MCF-7 | 0.3 |
| Breast ca. MDA-MB-231 | 43.5 |
| Breast ca. BT 549 | 0.1 |
| Breast ca. T47D | 0.1 |
| Breast ca. MDA-N | 0.7 |
| Breast Pool | 0.4 |
| Trachea | 9.7 |
| Lung | 0.0 |
| Fetal Lung | 0.6 |
| Lung ca. NCI-N417 | 0.0 |
| Lung ca. LX-1 | 0.1 |
| Lung ca. NCI-H146 | 0.3 |
| Lung ca. SHP-77 | 0.1 |
| Lung ca. A549 | 0.0 |
| Lung ca. NCI-H526 | 0.0 |
| Lung ca. NCI-H23 | 0.0 |
| Lung ca. NCI-H460 | 0.1 |
| Lung ca. HOP-62 | 2.0 |
| Lung ca. NCI-H522 | 0.3 |
| Liver | 2.2 |
| Fetal Liver | 3.4 |
| Liver ca. HepG2 | 2.3 |
| Kidney Pool | 1.7 |
| Fetal Kidney | 0.1 |
| Renal ca. 786-0 | 0.3 |
| Renal ca. A498 | 0.1 |
| Renal ca. ACHN | 0.0 |
| Renal ca. UO-31 | 0.6 |
| Renal ca. TK-10 | 1.0 |
| Bladder | 4.9 |
| Gastric ca. (liver met.) NCI-N87 | 1.4 |
| Gastric ca. KATO III | 0.2 |
| Colon ca. SW-948 | 0.0 |
| Colon ca. SW480 | 1.8 |
| Colon ca.* (SW480 met) SW620 | 0.1 |
| Colon ca. HT29 | 0.1 |
| Colon ca. HCT-116 | 2.5 |
| Colon ca. CaCo-2 | 0.5 |
| Colon cancer tissue | 9.7 |
| Colon ca. SW1116 | 0.0 |
| Colon ca. Colo-205 | 0.5 |
| Colon ca. SW-48 | 0.0 |
| Colon Pool | 0.7 |
| Small Intestine Pool | 0.3 |
| Stomach Pool | 0.5 |
| Bone Marrow Pool | 0.5 |
| Fetal Heart | 0.2 |
| Heart Pool | 0.4 |
| Lymph Node Pool | 0.7 |
| Fetal Skeletal Muscle | 0.7 |
| Skeletal Muscle Pool | 0.2 |
| Spleen Pool | 13.8 |
| Thymus Pool | 0.6 |
| CNS cancer (glio/astro) U87-MG | 2.2 |
| CNS cancer (glio/astro) U-118-MG | 25.5 |
| CNS cancer (neuro; met) SK-N-AS | 0.0 |
| CNS cancer (astro) SF-539 | 0.5 |
| CNS cancer (astro) SNB-75 | 12.7 |
| CNS cancer (glio) SNB-19 | 0.0 |
| CNS cancer (glio) SF-295 | 11.3 |
| Brain (Amygdala) Pool | 0.8 |
| Brain (cerebellum) | 1.1 |
| Brain (fetal) | 0.7 |
| Brain (Hippocampus) Pool | 0.7 |
| Cerebral Cortex Pool | 0.2 |
| Brain (Substantia nigra) Pool | 0.8 |
| Brain (Thalamus) Pool | 0.4 |
| Brain (whole) | 1.5 |
| Spinal Cord Pool | 0.4 |
| Adrenal Gland | 100.0 |
| Pituitary gland Pool | 0.1 |
| Salivary Gland | 59.0 |
| Thyroid (female) | 0.8 |
| Pancreatic ca. CAPAN2 | 57.8 |
| Pancreas Pool | 1.0 |

TABLE ND
Panel 4.1D

| | Rel. Exp. (%) |
|---|---|
| | Ag4971, |
| | Run |
| Tissue Name | 223692675 |
| Secondary Th1 act | 0.2 |
| Secondary Th2 act | 0.4 |
| Secondary Tr1 act | 0.9 |
| Secondary Th1 rest | 0.0 |
| Secondary Th2 rest | 0.7 |
| Secondary Tr1 rest | 0.2 |
| Primary Th1 act | 0.1 |
| Primary Th2 act | 0.4 |
| Primary Tr1 act | 0.1 |
| Primary Th1 rest | 0.0 |
| Primary Th2 rest | 0.0 |
| Primary Tr1 rest | 0.1 |
| CD45RA CD4 lymphocyte act | 0.1 |
| CD45RO CD4 lymphocyte act | 0.0 |
| CD8 lymphocyte act | 0.2 |
| Secondary CD8 lymphocyte rest | 0.0 |
| Secondary CD8 lymphocyte act | 0.0 |
| CD4 lymphocyte none | 0.1 |
| 2ry Th1/Th2/Tr1_anti-CD95 CH11 | 0.3 |
| LAK cells rest | 45.1 |
| LAK cells IL-2 | 0.1 |
| LAK cells IL-2 + IL-12 | 0.4 |
| LAK cells IL-2 + IFN gamma | 0.4 |
| LAK cells IL-2 + IL-18 | 0.2 |
| LAK cells PMA/ionomycin | 17.8 |
| NK Cells IL-2 rest | 0.1 |
| Two Way MLR 3 day | 4.6 |
| Two Way MLR 5 day | 1.7 |
| Two Way MLR 7 day | 0.4 |
| PBMC rest | 3.1 |
| PBMC PWM | 0.1 |
| PBMC PHA-L | 0.3 |
| Ramos (B cell) none | 0.0 |
| Ramos (B cell) ionomycin | 0.0 |
| B lymphocytes PWM | 0.2 |
| B lymphocytes CD40L and IL-4 | 0.6 |
| EOL-1 dbcAMP | 2.6 |
| EOL-1 dbcAMP PMA/ionomycin | 1.9 |
| Dendritic cells none | 59.9 |
| Dendritic cells LPS | 21.9 |
| Dendritic cells anti-CD40 | 100.0 |
| Monocytes rest | 32.3 |
| Monocytes LPS | 5.9 |
| Macrophages rest | 30.6 |
| Macrophages LPS | 8.7 |
| HUVEC none | 0.3 |
| HUVEC starved | 0.7 |
| HUVEC IL-1beta | 0.1 |
| HUVEC IFN gamma | 0.2 |
| HUVEC TNF alpha + IFN gamma | 0.1 |
| HUVEC TNF alpha + IL4 | 0.3 |
| HUVEC IL-11 | 0.1 |
| Lung Microvascular EC none | 0.0 |
| Lung Microvascular EC TNFalpha + IL-1beta | 2.2 |
| Microvascular Dermal EC none | 0.4 |
| Microsvasular Dermal EC TNFalpha + IL-1beta | 0.9 |
| Bronchial epithelium TNFalpha + IL1beta | 4.8 |
| Small airway epithelium none | 5.4 |
| Small airway epithelium TNFalpha + IL-1beta | 3.9 |
| Coronery artery SMC rest | 0.2 |
| Coronery artery SMC TNFalpha + IL-1beta | 0.2 |
| Astrocytes rest | 0.1 |
| Astrocytes TNFalpha + IL-1beta | 0.1 |
| KU-812 (Basophil) rest | 0.1 |
| KU-812 (Basophil) PMA/ionomycin | 3.0 |
| CCD1106 (Keratinocytes) none | 39.5 |
| CCD1106 (Keratinocytes) TNFalpha + IL-1beta | 28.5 |
| Liver cirrhosis | 0.4 |
| NCI-H292 none | 15.4 |
| NCI-H292 IL-4 | 10.3 |
| NCI-H292 IL-9 | 21.8 |
| NCI-H292 IL-13 | 8.1 |
| NCI-H292 IFN gamma | 17.7 |
| HPAEC none | 0.1 |
| HPAEC TNF alpha + IL-1 beta | 0.4 |
| Lung fibroblast none | 0.2 |
| Lung fibroblast TNF alpha + IL-1 beta | 0.2 |
| Lung fibroblast IL-4 | 0.1 |
| Lung fibroblast IL-9 | 0.3 |
| Lung fibroblast IL-13 | 0.5 |
| Lung fibroblast IFN gamma | 0.1 |
| Dermal fibroblast CCD1070 rest | 0.2 |
| Dermal fibroblast CCD1070 TNF alpha | 0.9 |
| Dermal fibroblast CCD1070 IL-1 beta | 0.3 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.7 |
| Colon | 1.5 |
| Lung | 7.3 |
| Thymus | 1.0 |
| Kidney | 0.1 |

AI_comprehensive panel_v1.0 Summary: Ag4971 Highest expression of this gene is detected in orthoarthritis (OA) bone (CT=26.7). High to moderate levels of expression of this gene is also seen in OA and adjacent normal bone and OA synovium. In addition, moderate to low levels of expression of this gene is also seen in bone, cartilage, synovium and synovial fluid samples derived from rheumatoid arthitis patient, OA cartilage, as well as, in samples derived from COPD lung, emphysema, atopic asthma, asthma, allergy, Crohn's disease (normal matched control and diseased), ulcerative colitis (normal matched control and diseased), and psoriasis (normal matched control and diseased). Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis.

General_screening_panel_v1.5 Summary: Ag4971 Highest expression of this gene is detected in adrenal gland (CT=27.8). Moderate to low levels of expression of this gene is also seen in tissues with metabolic/endocrine function such as pancreas, adipose, thyroid, and liver. Therefore, therapeutic modulation of the activity of this gene may prove useful in the treatment of endocrine/metabolically related diseases, such as obesity and diabetes.

Moderate levels of expression of this gene is also seen in number of cancer cell lines derived from melanoma, pancreatic, brain, colon, breast and prostate cancers. Therefore, expression of this gene may be used as diagnostic marker to detect the presence of these cancers. Furthermore, therapeutic modulation of this gene may be useful in the treatment of these cancers.

In addition, low levels of expression of this gene is also seen in whole and fetal brain, amygdala, cerebellum and substantia nigra. Therefore, therapeutic modulation of this gene product may be useful in the treatment of central nervous system disorders such as Alzheimer's disease, Parkinson's disease, epilepsy, multiple sclerosis, schizophrenia and depression.

Panel 4.1D Summary: Ag497I Highest expression of this gene is detected in anti-CD40 treated dendritic cells (CT=29). Moderate levels of expression of this gene is detected in dendritic cells, monocytes, macrophages, LAK cells, keratinocytes and mucoepidermoid NCI-H292 cells. Moderate to low levels of expression of this gene is also seen in PMA/ionomycin activated LAK cells, two way MLR, PBMC, eosinophils, small airway epithelium, TNFalpha+IL-1beta activated bronchial epithelium and microvascular dermal epithelium and lung. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

A. CG139363-01 and CG139363-02: Transmembrane Protein HTMP10-Like Protein.

Expression of gene CG 139363-01 and CG 139363-02 was assessed using the primer-probe set Ag4978, described in Table OA. Results of the RTQ-PCR runs are shown in Tables OB, OC and OD. Note that CG139363-02 represents a full-length physical clone.

TABLE OA

Probe Name Ag4978

| | | | Start | |
|---|---|---|---|---|
| Primers | Sequences | Length | Position | SEQ ID No |
| Forward | 5′-ggccctctcttggattagc-3′ | 19 | 134 | 300 |
| Probe | TET-5′-cacagccctgctggtggctttactat-3′-TAMRA | 26 | 177 | 301 |
| Reverse | 5′-cttcttcttcggtgaatcaaag-3′ | 22 | 206 | 302 |

TABLE OB
CNS_neurodegeneration_v1.0

| | Rel. Exp. (%) |
|---|---|
| | Ag4978, |
| | Run |
| Tissue Name | 224757409 |
| AD 1 Hippo | 4.1 |
| AD 2 Hippo | 8.7 |
| AD 3 Hippo | 2.6 |
| AD 4 Hippo | 9.1 |
| AD 5 Hippo | 11.3 |
| AD 6 Hippo | 29.9 |
| Control 2 Hippo | 43.8 |
| Control 4 Hippo | 11.3 |
| Control (Path) 3 Hippo | 4.9 |
| AD 1 Temporal Ctx | 7.2 |
| AD 2 Temporal Ctx | 23.2 |
| AD 3 Temporal Ctx | 2.5 |
| AD 4 Temporal Ctx | 33.0 |
| AD 5 Inf Temporal Ctx | 40.3 |
| AD 5 Sup Temporal Ctx | 17.3 |
| AD 6 Inf Temporal Ctx | 44.4 |
| AD 6 Sup Temporal Ctx | 34.9 |
| Control 1 Temporal Ctx | 5.1 |
| Control 2 Temporal Ctx | 41.8 |
| Control 3 Temporal Ctx | 23.3 |
| Control 3 Temporal Ctx | 12.8 |
| Control (Path) 1 Temporal Ctx | 49.7 |
| Control (Path) 2 Temporal Ctx | 31.4 |
| Control (Path) 3 Temporal Ctx | 4.4 |
| Control (Path) 4 Temporal Ctx | 20.4 |
| AD 1 Occipital Ctx | 6.2 |
| AD 2 Occipital Ctx (Missing) | 0.0 |
| AD 3 Occipital Ctx | 1.8 |
| AD 4 Occipital Ctx | 24.7 |
| AD 5 Occipital Ctx | 40.3 |
| AD 6 Occipital Ctx | 23.7 |
| Control 1 Occipital Ctx | 4.4 |
| Control 2 Occipital Ctx | 43.8 |
| Control 3 Occipital Ctx | 20.0 |
| Control 4 Occipital Ctx | 9.0 |
| Control (Path) 1 Occipital Ctx | 100.0 |
| Control (Path) 2 Occipital Ctx | 15.8 |
| Control (Path) 3 Occipital Ctx | 4.4 |
| Control (Path) 4 Occipital Ctx | 13.3 |
| Control 1 Parietal Ctx | 7.7 |
| Control 2 Parietal Ctx | 23.7 |
| Control 3 Parietal Ctx | 19.3 |
| Control (Path) 1 Parietal Ctx | 57.8 |
| Control (Path) 2 Parietal Ctx | 20.4 |
| Control (Path) 3 Parietal Ctx | 4.1 |
| Control (Path) 4 Parietal Ctx | 25.9 |

TABLE OC
General_screening_panel_v1.5

| | Rel. Exp. (%) |
|---|---|
| | Ag4978, |
| | Run |
| Tissue Name | 228940920 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.1 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 2.8 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 62.9 |
| Brain (cerebellum) | 25.9 |
| Brain (fetal) | 0.0 |
| Brain (Hippocampus) Pool | 51.8 |
| Cerebral Cortex Pool | 66.0 |
| Brain (Substantia nigra) Pool | 48.6 |
| Brain (Thalamus) Pool | 100.0 |
| Brain (whole) | 64.6 |
| Spinal Cord Pool | 24.3 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

TABLE OD
Panel 4.1D

| | Rel. Exp. (%) | | Rel. Exp. (%) |
|---|---|---|---|
| | Ag4978, Run | | Ag4978, Run |
| Tissue Name | 223693384 | Tissue Name | 223693384 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN | 0.0 |
| | | gamma | |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC | 0.0 |
| | | TNF alpha + IL-1beta | |
| Primary Th2 act | 0.0 | Microvascular Dermal EC | 0.0 |
| | | none | |
| Primary Tr1 act | 0.0 | Microsvasular Dermal EC | 0.0 |
| | | TNF alpha + IL-1beta | |
| Primary Th1 rest | 0.0 | Bronchial epithelium | 0.6 |
| | | TNF alpha + IL1beta | |
| Primary Th2 rest | 0.0 | Small airway epithelium none | 0.0 |
| Primary Tr1 rest | 0.0 | Small airway epithelium | 0.0 |
| | | TNF alpha + IL-1beta | |
| CD45RA CD4 | 0.0 | Coronery artery SMC rest | 0.0 |
| lymphocyte act | | | |
| CD45RO CD4 | 0.0 | Coronery artery SMC | 0.0 |
| lymphocyte act | | TNF alpha + IL-1beta | |
| CD8 lymphocyte act | 0.0 | Astrocytes rest | 0.0 |
| Secondary CD8 | 0.0 | Astrocytes TNF alpha + IL- | 0.0 |
| lymphocyte rest | | 1beta | |
| Secondary CD8 | 0.0 | KU-812 (Basophil) rest | 0.0 |
| lymphocyte act | | | |
| CD4 lymphocyte none | 0.0 | KU-812 (Basophil) | 0.0 |
| | | PMA/ionomycin | |
| 2ry Th1/Th2/Tr1_anti- | 0.0 | CCD1106 (Keratinocytes) | 0.0 |
| CD95 CH11 | | none | |
| LAK cells rest | 1.0 | CCD1106 (Keratinocytes) | 0.7 |
| | | TNF alpha + IL-1beta | |
| LAK cells IL-2 | 0.0 | Liver cirrhosis | 0.0 |
| LAK cells IL-2 + IL-12 | 0.0 | NCI-H292 none | 0.0 |
| LAK cells IL-2 + IFN | 0.0 | NCI-H292 IL-4 | 0.0 |
| gamma | | | |
| LAK cells IL-2 + IL-18 | 0.0 | NCI-H292 IL-9 | 0.0 |
| LAK cells | 0.0 | NCI-H292 IL-13 | 0.0 |
| PMA/ionomycin | | | |
| NK Cells IL-2 rest | 0.0 | NCI-H292 IFN gamma | 0.5 |
| Two Way MLR 3 day | 0.0 | HPAEC none | 0.6 |
| Two Way MLR 5 day | 0.0 | HPAEC TNF alpha + IL-1 | 0.0 |
| | | beta | |
| Two Way MLR 7 day | 0.0 | Lung fibroblast none | 0.0 |
| PBMC rest | 0.0 | Lung fibroblast TNF alpha + IL-1 | 0.0 |
| | | beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 | 0.0 |
| | | rest | |
| B lymphocytes CD40L | 0.0 | Dermal fibroblast CCD1070 | 0.0 |
| and IL-4 | | TNF alpha | |
| EOL-1 dbcAMP | 0.0 | Dermal fibroblast CCD1070 | 0.0 |
| | | IL-1beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 0.5 |
| Macrophages LPS | 0.0 | Thymus | 100.0 |
| HUVEC none | 0.0 | Kidney | 0.6 |
| HUVEC starved | 0.0 | | |

CNS_neurodegeneration_v1.0 Summary: Ag4978 This panel does not show differential expression of this gene in Alzheimer's disease. However, this profile confirms the expression of this gene at moderate levels in the brain. See Panel 1.5 for discussion of this gene in the central nervous system.

General_screening_panel_v1.5 Summary: Ag4978 Highest expression of this gene is seen in the thalamus (CT=26.7). Overall, expression of this gene appears to be highly associated with the brain. High levels of expression are seen in all regions of the CNS examined, including the hippocampus, thalamus, substantia nigra, amygdala, cerebellum and cerebral cortex. Therefore, therapeutic modulation of the expression or function of this gene may be useful in the treatment of neurological disorders, such as Alzheimer's disease, Parkinson's disease, schizophrenia, multiple sclerosis, stroke and epilepsy.

Panel 4.1D Summary: Ag4978 This transcript-is expressed at significant levels only in the thymus (CT=30.2). The putative protein encoded by thus gene could therefore play an important role in T cell development. Therapeutic modulation of the expression or function of this gene may modulate immune function (T cell development) and be important for organ transplant, AIDS treatment or post chemotherapy immune reconstitution.

P. CG140188-01: DC2-Like Protein.

Expression of gene CG140188-01 was assessed using the primer-probe set Ag7417, described in Table PA. Results of the RTQ-PCR runs are shown in Table PB.

TABLE PA

Probe Name Ag7417

| | | | Start | |
|---|---|---|---|---|
| Primers | Sequences | Length | Position | SEQ ID No |
| Forward | 5′-cattggctctatgactgatgaac-3′ | 23 | 194 | 303 |
| Probe | TET-5′-ccaagaaagctactggcctctgat-3′-TAMRA | 24 | 223 | 304 |
| Reverse | 5′-ggatgcaagtccttccataata-3′ | 22 | 269 | 305 |

TABLE PB
Panel 4.1D

| | Rel. Exp. (%) | | Rel. Exp. (%) |
|---|---|---|---|
| | Ag7417, Run | | Ag7417, Run |
| Tissue Name | 305065593 | Tissue Name | 305065593 |
| Secondary Th1 act | 21.0 | HUVEC IL-1beta | 48.0 |
| Secondary Th2 act | 29.5 | HUVEC IFN gamma | 37.9 |
| Secondary Tr1 act | 14.6 | HUVEC TNF alpha + IFN | 18.8 |
| | | gamma | |
| Secondary Th1 rest | 1.2 | HUVEC TNF alpha + IL4 | 24.8 |
| Secondary Th2 rest | 3.6 | HUVEC IL-11 | 22.2 |
| Secondary Tr1 rest | 4.2 | Lung Microvascular EC none | 100.0 |
| Primary Th1 act | 3.4 | Lung Microvascular EC | 41.8 |
| | | TNF alpha + IL-1beta | |
| Primary Th2 act | 24.0 | Microvascular Dermal EC | 9.5 |
| | | none | |
| Primary Tr1 act | 20.6 | Microsvasular Dermal EC | 18.7 |
| | | TNF alpha + IL-1beta | |
| Primary Th1 rest | 1.4 | Bronchial epithelium | 22.4 |
| | | TNF alpha + IL1beta | |
| Primary Th2 rest | 2.5 | Small airway epithelium none | 9.2 |
| Primary Tr1 rest | 1.2 | Small airway epithelium | 14.9 |
| | | TNF alpha + IL-1beta | |
| CD45RA CD4 | 22.8 | Coronery artery SMC rest | 49.0 |
| lymphocyte act | | | |
| CD45RO CD4 | 21.3 | Coronery artery SMC | 45.1 |
| lymphocyte act | | TNF alpha + IL-1beta | |
| CD8 lymphocyte act | 13.8 | Astrocytes rest | 10.7 |
| Secondary CD8 | 2.2 | Astrocytes TNF alpha + IL- | 24.3 |
| lymphocyte rest | | 1beta | |
| Secondary CD8 | 6.3 | KU-812 (Basophil) rest | 22.8 |
| lymphocyte act | | | |
| CD4 lymphocyte none | 1.6 | KU-812 (Basophil) | 31.4 |
| | | PMA/ionomycin | |
| 2ry Th1/Th2/Tr1_anti- | 3.2 | CCD1106 (Keratinocytes) | 23.8 |
| CD95 CH11 | | none | |
| LAK cells rest | 6.5 | CCD1106 (Keratinocytes) | 10.7 |
| | | TNF alpha + IL-1beta | |
| LAK cells IL-2 | 4.2 | Liver cirrhosis | 11.3 |
| LAK cells IL-2 + IL-12 | 1.4 | NCI-H292 none | 25.2 |
| LAK cells IL-2 + IFN | 5.8 | NCI-H292 IL-4 | 17.3 |
| gamma | | | |
| LAK cells IL-2 + IL-18 | 2.6 | NCI-H292 IL-9 | 33.2 |
| LAK cells | 9.5 | NCI-H292 IL-13 | 20.6 |
| PMA/ionomycin | | | |
| NK Cells IL-2 rest | 20.9 | NCI-H292 IFN gamma | 6.8 |
| Two Way MLR 3 day | 4.8 | HPAEC none | 15.2 |
| Two Way MLR 5 day | 2.5 | HPAEC TNF alpha + IL-1 | 54.3 |
| | | beta | |
| Two Way MLR 7 day | 4.3 | Lung fibroblast none | 22.2 |
| PBMC rest | 1.5 | Lung fibroblast TNF alpha + IL-1 | 21.0 |
| | | beta | |
| PBMC PWM | 5.1 | Lung fibroblast IL-4 | 27.5 |
| PBMC PHA-L | 3.6 | Lung fibroblast IL-9 | 30.1 |
| Ramos (B cell) none | 36.3 | Lung fibroblast IL-13 | 15.2 |
| Ramos (B cell) ionomycin | 59.0 | Lung fibroblast IFN gamma | 38.4 |
| B lymphocytes PWM | 3.9 | Dermal fibroblast CCD1070 | 40.1 |
| | | rest | |
| B lymphocytes CD40L | 10.4 | Dermal fibroblast CCD1070 | 49.7 |
| and IL-4 | | TNF alpha | |
| EOL-1 dbcAMP | 26.6 | Dermal fibroblast CCD1070 | 31.9 |
| | | IL-1beta | |
| EOL-1 dbcAMP | 14.1 | Dermal fibroblast IFN gamma | 17.0 |
| PMA/ionomycin | | | |
| Dendritic cells none | 10.2 | Dermal fibroblast IL-4 | 28.7 |
| Dendritic cells LPS | 5.1 | Dermal Fibroblasts rest | 8.4 |
| Dendritic cells anti-CD40 | 3.5 | Neutrophils TNFa + LPS | 0.0 |
| Monocytes rest | 5.4 | Neutrophils rest | 1.2 |
| Monocytes LPS | 22.7 | Colon | 0.7 |
| Macrophages rest | 5.6 | Lung | 1.4 |
| Macrophages LPS | 3.7 | Thymus | 4.1 |
| HUVEC none | 43.8 | Kidney | 6.0 |
| HUVEC starved | 38.2 | | |

CNS_neurodegeneration_v1.0 Summary: Ag7417 Expression of this gene is low/undetectable in all samples on this panel (CTs>35).

Panel 4.1D Summary: Ag7417 Highest expression of this gene is seen in untreated lung microvascular endothelial cells (CT=30.8). This gene is also expressed at moderate levels in a wide range of cell types of significance in the immune response in health and disease. These cells include members of the T-cell, endothelial cell, basophil, astrocyte, monocyte, and peripheral blood mononuclear cell family, as well as epithelial and fibroblast cell types from lung and skin. This ubiquitous pattern of expression suggests that this gene product may be involved in homeostatic processes for these and other cell types and tissues as well as in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Q. CG140305-01: Complement-c1q Tumor Necrosis Factor-Related Protein-Like Protein.

Expression of gene CG140305-01 was assessed using the primer-probe set Ag6486, described in Table QA. Results of the RTQ-PCR runs are shown in Tables QB, QC and QD.

TABLE QA

Probe Name Ag6486

| | | | Start | |
|---|---|---|---|---|
| Primers | Sequences | Length | Position | SEQ ID No |
| Forward | 5′-tgctggatgtatctgattgc-3′ | 21 | 581 | 306 |
| Probe | TET-5′-caacacagtcttcagcatgtacagct-3′-TAMRA | 26 | 543 | 307 |
| Reverse | 5′-gtatgtgtaccttatgcacaatgg-3′ | 24 | 519 | 308 |

TABLE QB
General_screening_panel_v1.6

| | Rel. Exp. (%) | | Rel. Exp. (%) |
|---|---|---|---|
| | Ag6486, Run | | Ag6486, Run |
| Tissue Name | 277240051 | Tissue Name | 277240051 |
| Adipose | 13.9 | Renal ca. TK-10 | 12.4 |
| Melanoma* Hs688(A).T | 35.4 | Bladder | 14.7 |
| Melanoma* Hs688(B).T | 55.9 | Gastric ca. (liver met.) | 10.8 |
| | | NCI-N87 | |
| Melanoma* M14 | 1.4 | Gastric ca. KATO III | 0.3 |
| Melanoma* LOXIMVI | 1.3 | Colon ca. SW-948 | 0.4 |
| Melanoma* SK-MEL-5 | 2.0 | Colon ca. SW480 | 0.6 |
| Squamous cell carcinoma | 1.7 | Colon ca.* (SW480 met) | 4.4 |
| SCC-4 | | SW620 | |
| Testis Pool | 30.8 | Colon ca. HT29 | 2.6 |
| Prostate ca.* (bone met) | 2.8 | Colon ca. HCT-116 | 4.9 |
| PC-3 | | | |
| Prostate Pool | 15.2 | Colon ca. CaCo-2 | 9.0 |
| Placenta | 2.6 | Colon cancer tissue | 33.7 |
| Uterus Pool | 3.8 | Colon ca. SW1116 | 1.7 |
| Ovarian ca. OVCAR-3 | 4.4 | Colon ca. Colo-205 | 2.0 |
| Ovarian ca. SK-OV-3 | 14.0 | Colon ca. SW-48 | 1.0 |
| Ovarian ca. OVCAR-4 | 1.0 | Colon Pool | 9.1 |
| Ovarian ca. OVCAR-5 | 9.1 | Small Intestine Pool | 22.8 |
| Ovarian ca. IGROV-1 | 4.6 | Stomach Pool | 15.5 |
| Ovarian ca. OVCAR-8 | 1.2 | Bone Marrow Pool | 4.6 |
| Ovary | 3.1 | Fetal Heart | 10.5 |
| Breast ca. MCF-7 | 7.2 | Heart Pool | 3.9 |
| Breast ca. MDA-MB-231 | 5.0 | Lymph Node Pool | 5.8 |
| Breast ca. BT 549 | 4.2 | Fetal Skeletal Muscle | 39.5 |
| Breast ca. T47D | 0.5 | Skeletal Muscle Pool | 1.8 |
| Breast ca. MDA-N | 1.1 | Spleen Pool | 4.4 |
| Breast Pool | 11.0 | Thymus Pool | 14.3 |
| Trachea | 26.8 | CNS cancer (glio/astro) | 2.2 |
| | | U87-MG | |
| Lung | 4.7 | CNS cancer (glio/astro) | 4.5 |
| | | U-118-MG | |
| Fetal Lung | 34.9 | CNS cancer (neuro; met) | 3.8 |
| | | SK-N-AS | |
| Lung ca. NCI-N417 | 0.3 | CNS cancer (astro) SF- | 2.2 |
| | | 539 | |
| Lung ca. LX-1 | 6.8 | CNS cancer (astro) SNB- | 8.8 |
| | | 75 | |
| Lung ca. NCI-H146 | 12.9 | CNS cancer (glio) SNB- | 3.7 |
| | | 19 | |
| Lung ca. SHP-77 | 19.8 | CNS cancer (glio) SF-295 | 7.6 |
| Lung ca. A549 | 0.7 | Brain (Amygdala) Pool | 9.1 |
| Lung ca. NCI-H526 | 2.5 | Brain (cerebellum) | 100.0 |
| Lung ca. NCI-H23 | 9.4 | Brain (fetal) | 20.2 |
| Lung ca. NCI-H460 | 1.0 | Brain (Hippocampus) | 13.5 |
| | | Pool | |
| Lung ca. HOP-62 | 4.9 | Cerebral Cortex Pool | 10.8 |
| Lung ca. NCI-H522 | 1.2 | Brain (Substantia nigra) | 7.4 |
| | | Pool | |
| Liver | 0.4 | Brain (Thalamus) Pool | 14.7 |
| Fetal Liver | 5.7 | Brain (whole) | 8.0 |
| Liver ca. HepG2 | 5.2 | Spinal Cord Pool | 24.8 |
| Kidney Pool | 20.2 | Adrenal Gland | 3.2 |
| Fetal Kidney | 65.1 | Pituitary gland Pool | 2.2 |
| Renal ca. 786-0 | 10.4 | Salivary Gland | 18.7 |
| Renal ca. A498 | 2.1 | Thyroid (female) | 2.1 |
| Renal ca. ACHN | 2.2 | Pancreatic ca. CAPAN2 | 5.6 |
| Renal ca. UO-31 | 1.2 | Pancreas Pool | 3.3 |

TABLE QC
Panel 4.1D

| | Rel. Exp. (%) | | Rel. Exp. (%) |
|---|---|---|---|
| | Ag6486, Run | | Ag6486, Run |
| Tissue Name | 269282929 | Tissue Name | 269282929 |
| Secondary Th1 act | 15.8 | HUVEC IL-1beta | 9.7 |
| Secondary Th2 act | 27.9 | HUVEC IFN gamma | 8.8 |
| Secondary Tr1 act | 17.0 | HUVEC TNF alpha + IFN | 7.5 |
| | | gamma | |
| Secondary Th1 rest | 9.5 | HUVEC TNF alpha + IL4 | 5.3 |
| Secondary Th2 rest | 9.4 | HUVEC IL-11 | 8.5 |
| Secondary Tr1 rest | 8.8 | Lung Microvascular EC none | 92.0 |
| Primary Th1 act | 3.8 | Lung Microvascular EC | 5.8 |
| | | TNF alpha + IL-1beta | |
| Primary Th2 act | 38.2 | Microvascular Dermal EC | 9.4 |
| | | none | |
| Primary Tr1 act | 31.0 | Microsvasular Dermal EC | 8.5 |
| | | TNF alpha + IL-1beta | |
| Primary Th1 rest | 9.5 | Bronchial epithelium | 5.4 |
| | | TNF alpha + IL1beta | |
| Primary Th2 rest | 13.2 | Small airway epithelium none | 0.0 |
| Primary Tr1 rest | 1.4 | Small airway epithelium | 6.4 |
| | | TNF alpha + IL-1beta | |
| CD45RA CD4 | 34.6 | Coronery artery SMC rest | 3.9 |
| lymphocyte act | | | |
| CD45RO CD4 | 40.3 | Coronery artery SMC | 5.4 |
| lymphocyte act | | TNF alpha + IL-1beta | |
| CD8 lymphocyte act | 21.9 | Astrocytes rest | 8.0 |
| Secondary CD8 | 6.9 | Astrocytes TNF alpha + IL- | 0.0 |
| lymphocyte rest | | 1beta | |
| Secondary CD8 | 5.2 | KU-812 (Basophil) rest | 52.1 |
| lymphocyte act | | | |
| CD4 lymphocyte none | 13.5 | KU-812 (Basophil) | 33.2 |
| | | PMA/ionomycin | |
| 2ry Th1/Th2/Tr1_anti- | 24.1 | CCD1106 (Keratinocytes) | 8.0 |
| CD95 CH11 | | none | |
| LAK cells rest | 8.0 | CCD1106 (Keratinocytes) | 5.7 |
| | | TNF alpha + IL-1beta | |
| LAK cells IL-2 | 10.4 | Liver cirrhosis | 20.7 |
| LAK cells IL-2 + IL-12 | 1.9 | NCI-H292 none | 34.6 |
| LAK cells IL-2 + IFN | 14.3 | NCI-H292 IL-4 | 24.3 |
| gamma | | | |
| LAK cells IL-2 + IL-18 | 21.0 | NCI-H292 IL-9 | 34.4 |
| LAK cells | 7.0 | NCI-H292 IL-13 | 29.9 |
| PMA/ionomycin | | | |
| NK Cells IL-2 rest | 70.2 | NCI-H292 IFN gamma | 17.2 |
| Two Way MLR 3 day | 23.2 | HPAEC none | 7.0 |
| Two Way MLR 5 day | 2.0 | HPAEC TNF alpha + IL-1 | 8.0 |
| | | beta | |
| Two Way MLR 7 day | 6.9 | Lung fibroblast none | 5.0 |
| PBMC rest | 1.6 | Lung fibroblast TNF alpha + IL-1 | 9.6 |
| | | beta | |
| PBMC PWM | 7.2 | Lung fibroblast IL-4 | 0.0 |
| PBMC PHA-L | 15.2 | Lung fibroblast IL-9 | 5.3 |
| Ramos (B cell) none | 9.7 | Lung fibroblast IL-13 | 0.0 |
| Ramos (B cell) ionomycin | 17.2 | Lung fibroblast IFN gamma | 14.1 |
| B lymphocytes PWM | 6.8 | Dermal fibroblast CCD1070 | 12.9 |
| | | rest | |
| B lymphocytes CD40L | 56.6 | Dermal fibroblast CCD1070 | 100.0 |
| and IL-4 | | TNF alpha | |
| EOL-1 dbcAMP | 50.0 | Dermal fibroblast CCD1070 | 8.0 |
| | | IL-1beta | |
| EOL-1 dbcAMP | 8.1 | Dermal fibroblast IFN gamma | 9.2 |
| PMA/ionomycin | | | |
| Dendritic cells none | 13.2 | Dermal fibroblast IL-4 | 28.1 |
| Dendritic cells LPS | 3.1 | Dermal Fibroblasts rest | 7.9 |
| Dendritic cells anti-CD40 | 3.6 | Neutrophils TNFa + LPS | 2.9 |
| Monocytes rest | 0.0 | Neutrophils rest | 6.2 |
| Monocytes LPS | 7.7 | Colon | 46.0 |
| Macrophages rest | 2.4 | Lung | 9.0 |
| Macrophages LPS | 0.0 | Thymus | 51.4 |
| HUVEC none | 1.7 | Kidney | 78.5 |
| HUVEC starved | 22.7 | | |

TABLE QD
Panel CNS_1.1

| | Rel. Exp. (%) | | Rel. Exp. (%) |
|---|---|---|---|
| | Ag6486, | | Ag6486, |
| Tissue Name | Run 271481506 | Tissue Name | Run 271481506 |
| Cing Gyr Depression2 | 26.8 | BA17 PSP2 | 5.6 |
| Cing Gyr Depression | 13.8 | BA17 PSP | 11.3 |
| Cing Gyr PSP2 | 4.8 | BA17 Huntington's2 | 17.2 |
| Cing Gyr PSP | 52.9 | BA17 Huntington's | 20.0 |
| Cing Gyr | 33.2 | BA17 Parkinson's2 | 26.4 |
| Huntington's2 | | | |
| Cing Gyr Huntington's | 53.2 | BA17 Parkinson's | 31.4 |
| Cing Gyr Parkinson's2 | 0.0 | BA17 Alzheimer's2 | 7.5 |
| Cing Gyr Parkinson's | 53.6 | BA17 Control2 | 12.6 |
| Cing Gyr Alzheimer's2 | 14.6 | BA17 Control | 19.8 |
| Cing Gyr Alzheimer's | 23.5 | BA9 Depression2 | 4.9 |
| Cing Gyr Control2 | 16.6 | BA9 Depression | 0.4 |
| Cing Gyr Control | 52.9 | BA9 PSP2 | 6.0 |
| Temp Pole | 15.6 | BA9 PSP | 15.6 |
| Depression2 | | | |
| Temp Pole PSP2 | 0.0 | BA9 Huntington's2 | 28.3 |
| Temp Pole PSP | 2.1 | BA9 Huntington's | 36.3 |
| Temp Pole | 28.3 | BA9 Parkinson's2 | 26.4 |
| Huntington's | | | |
| Temp Pole | 31.4 | BA9 Parkinson's | 32.8 |
| Parkinson's2 | | | |
| Temp Pole Parkinson's | 16.2 | BA9 Alzheimer's2 | 4.6 |
| Temp Pole | 5.9 | BA9 Alzheimer's | 1.8 |
| Alzheimer's2 | | | |
| Temp Pole Alzheimer's | 4.7 | BA9 Control2 | 59.9 |
| Temp Pole Control2 | 33.9 | BA9 Control | 14.3 |
| Temp Pole Control | 4.0 | BA7 Depression | 17.4 |
| Glob Palladus | 12.1 | BA7 PSP2 | 12.1 |
| Depression | | | |
| Glob Palladus PSP2 | 7.0 | BA7 PSP | 14.5 |
| Glob Palladus PSP | 14.7 | BA7 Huntington's2 | 76.8 |
| Glob Palladus | 30.4 | BA7 Huntington's | 26.1 |
| Parkinson's2 | | | |
| Glob Palladus | 73.2 | BA7 Parkinson's2 | 17.4 |
| Parkinson's | | | |
| Glob Palladus | 7.4 | BA7 Parkinson's | 19.3 |
| Alzheimer's2 | | | |
| Glob Palladus | 9.1 | BA7 Alzheimer's2 | 2.8 |
| Alzheimer's | | | |
| Glob Palladus Control2 | 6.7 | BA7 Control2 | 14.4 |
| Glob Palladus Control | 19.5 | BA7 Control | 9.9 |
| Sub Nigra Depression2 | 9.3 | BA4 Depression2 | 6.7 |
| Sub Nigra Depression | 4.5 | BA4 Depression | 13.5 |
| Sub Nigra PSP2 | 17.4 | BA4 PSP2 | 10.2 |
| Sub Nigra | 46.0 | BA4 PSP | 7.5 |
| Huntington's2 | | | |
| Sub Nigra Huntington's | 63.7 | BA4 Huntington's2 | 13.5 |
| Sub Nigra Parkinson's2 | 76.3 | BA4 Huntington's | 14.5 |
| Sub Nigra | 27.4 | BA4 Parkinson's2 | 34.2 |
| Alzheimer's2 | | | |
| Sub Nigra Control2 | 36.9 | BA4 Parkinson's | 39.2 |
| Sub Nigra Control | 100.0 | BA4 Alzheimer's2 | 2.3 |
| BA17 Depression2 | 25.3 | BA4 Control2 | 22.5 |
| BA17 Depression | 19.2 | BA4 Control | 15.0 |

General_screening_panel_v1.6 Summary: Ag6486 Highest expression of this gene is detected in brain cerebellum (CT=27.8). In addition, moderate levels of expression of this gene is also seen in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, fetal liver and the gastrointestinal tract. This gene encodes a splice variant of the complement C1q tumor necrosis factor-related protein, a member of the C1q family. This family includes proteins such as complement subunit C1q, adiponectin, gliacolin, C1q-related protein, cerebellin, CORS26 etc., all of which are secreted. These proteins have been implicated in tissue differentiation, immune regulation, energy homeostasis, synaptic function and in diseases such as obesity, diabetes and neurodegeneration. Adiponectin, a member of C1q family and protein closely related to complement C1q tumor necrosis factor-related protein, is induced over 100-fold in adipocyte differentiation (Scherer et al., 1995, J Biol Chem 270(45):26746-9 PMID: 7592907) and is involved in adipocyte signaling (Hu et al., 1996, J Biol Chem 271(18): 10697-703 PMID: 8631877). Recently, adiponectin has been shown to reverse insulin resistance in mouse models of lipoatrophy and obesity (Yamauchi et al., 2001, Nat Med 7(8):941-6 PMID: 11479627). Therefore this protein, and proteins related to it, are potential antigens for development protein therapeutics for use in the treatment of obesity and type II diabetes.

This gene is expressed at much higher levels in fetal (CTs=29-32) when compared to adult skeletal muscle, lung and liver (CTs=32-35.9). This observation suggests that expression of this gene can be used to distinguish fetal from adult skeletal muscle, lung and liver. In addition, the relative overexpression of this gene in fetal tissue suggests that the protein product may enhance growth or development of these tissues in the fetus and thus may also act in a regenerative capacity in the adult. Therefore, therapeutic modulation of the protein encoded by this gene could be useful in treatment of muscle, lung and liver related diseases.

Panel 4.1D Summary: Ag6486 Highest expression of this gene is detected in TNF alpha treated dermal fibroblast (CT=32.4). In addition, moderate to low levels of expression of this gene is also seen in activated T cells, IL-2 treated NK Cells, CD40L and IL-4 treated B lymphocytes, eosinophils, lung microvascular endothelial cells, basophils, NCI-H292 mucoepidermoid cells, and normal tissues represented by colon, thymus and kidney. Therefore, therapeutic modulation of the activity of this gene or its protein product, through the use of protein therapeutics or antibodies, might be beneficial in the treatment of autoimmune and inflammatory diseases that involve these cell and tissue types, such as lupus erythematosus, asthma, emphysema, Crohn's disease, ulcerative colitis, rheumatoid arthritis, osteoarthritis, and psoriasis.

Panel CNS_{—}1.1 Summary: Ag6486 This panel confirms the expression of this gene at low levels in the brains of an independent group of individuals. See Panel 1.6 for a discussion of this gene in treatment of central nervous system disorders.

R. CG140639-01 and CG140639-02: Flotillin-2 (Reggie-1) (REG-1)-Like Protein.

Expression of gene CG 140639-01 and CG 140639-02 was assessed using the primer-probe set Ag5036, described in Table RA. Results of the RTQ-PCR runs are shown in Tables RB and RC. Note that CG 140639-02 represents a full-length physical clone.

TABLE RA

Probe Name Ag5036

| | | | | |
|---|---|---|---|---|
| Primers | Sequences | Length | Start Position | SEQ ID No |

| | | | | |
|---|---|---|---|---|
| Forward | 5′-gggtaagaatgtgcaggacat-3′ | 21 | 349 | 309 |
| Probe | TET-5′-aaaacgtcgtcctgcagaccctg-3′-TAMRA | 23 | 372 | 310 |
| Reverse | 5′-tgataaatctgctccactgtca-3′ | 22 | 426 | 311 |

TABLE RB
General_screening_panel_v1.5

| | Rel. Exp. (%) | | Rel. Exp. (%) |
|---|---|---|---|
| | Ag5036, Run | | Ag5036, Run |
| Tissue Name | 228967203 | Tissue Name | 228967203 |
| Adipose | 10.4 | Renal ca. TK-10 | 44.4 |
| Melanoma* Hs688(A).T | 23.0 | Bladder | 33.4 |
| Melanoma* Hs688(B).T | 18.4 | Gastric ca. (liver met.) | 25.9 |
| | | NCI-N87 | |
| Melanoma* M14 | 45.4 | Gastric ca. KATO III | 41.5 |
| Melanoma* LOXIMVI | 13.9 | Colon ca. SW-948 | 14.5 |
| Melanoma* SK-MEL-5 | 23.2 | Colon ca. SW480 | 61.6 |
| Squamous cell carcinoma | 6.1 | Colon ca.* (SW480 met) | 47.3 |
| SCC-4 | | SW620 | |
| Testis Pool | 6.3 | Colon ca. HT29 | 37.1 |
| Prostate ca.* (bone met) | 15.4 | Colon ca. HCT-116 | 39.5 |
| PC-3 | | | |
| Prostate Pool | 17.2 | Colon ca. CaCo-2 | 68.3 |
| Placenta | 33.7 | Colon cancer tissue | 20.3 |
| Uterus Pool | 11.0 | Colon ca. SW1116 | 8.9 |
| Ovarian ca. OVCAR-3 | 57.0 | Colon ca. Colo-205 | 12.1 |
| Ovarian ca. SK-OV-3 | 100.0 | Colon ca. SW-48 | 11.7 |
| Ovarian ca. OVCAR-4 | 27.5 | Colon Pool | 15.3 |
| Ovarian ca. OVCAR-5 | 39.8 | Small Intestine Pool | 11.3 |
| Ovarian ca. IGROV-1 | 44.4 | Stomach Pool | 8.2 |
| Ovarian ca. OVCAR-8 | 26.1 | Bone Marrow Pool | 4.8 |
| Ovary | 9.3 | Fetal Heart | 16.4 |
| Breast ca. MCF-7 | 24.8 | Heart Pool | 9.9 |
| Breast ca. MDA-MB-231 | 89.5 | Lymph Node Pool | 12.7 |
| Breast ca. BT 549 | 47.6 | Fetal Skeletal Muscle | 11.0 |
| Breast ca. T47D | 16.2 | Skeletal Muscle Pool | 22.2 |
| Breast ca. MDA-N | 11.7 | Spleen Pool | 14.6 |
| Breast Pool | 10.7 | Thymus Pool | 10.9 |
| Trachea | 22.7 | CNS cancer (glio/astro) | 39.8 |
| | | U87-MG | |
| Lung | 2.2 | CNS cancer (glio/astro) | 23.0 |
| | | U-118-MG | |
| Fetal Lung | 29.9 | CNS cancer (neuro; met) | 19.6 |
| | | SK-N-AS | |
| Lung ca. NCI-N417 | 6.6 | CNS cancer (astro) SF- | 13.9 |
| | | 539 | |
| Lung ca. LX-1 | 68.8 | CNS cancer (astro) SNB- | 34.4 |
| | | 75 | |
| Lung ca. NCI-H146 | 13.4 | CNS cancer (glio) SNB- | 43.8 |
| | | 19 | |
| Lung ca. SHP-77 | 41.2 | CNS cancer (glio) SF-295 | 48.0 |
| Lung ca. A549 | 52.1 | Brain (Amygdala) Pool | 18.2 |
| Lung ca. NCI-H526 | 23.7 | Brain (cerebellum) | 47.0 |
| Lung ca. NCI-H23 | 44.1 | Brain (fetal) | 27.5 |
| Lung ca. NCI-H460 | 29.1 | Brain (Hippocampus) | 15.8 |
| | | Pool | |
| Lung ca. HOP-62 | 43.8 | Cerebral Cortex Pool | 23.5 |
| Lung ca. NCI-H522 | 36.9 | Brain (Substantia nigra) | 18.6 |
| | | Pool | |
| Liver | 5.8 | Brain (Thalamus) Pool | 22.8 |
| Fetal Liver | 47.6 | Brain (whole) | 23.2 |
| Liver ca. HepG2 | 15.3 | Spinal Cord Pool | 11.7 |
| Kidney Pool | 20.9 | Adrenal Gland | 13.8 |
| Fetal Kidney | 8.7 | Pituitary gland Pool | 2.7 |
| Renal ca. 786-0 | 21.9 | Salivary Gland | 14.1 |
| Renal ca. A498 | 19.1 | Thyroid (female) | 11.3 |
| Renal ca. ACHN | 50.0 | Pancreatic ca. CAPAN2 | 21.8 |
| Renal ca. UO-31 | 39.2 | Pancreas Pool | 18.2 |

TABLE RC
Panel 4.1D

| | Rel. Exp. (%) | | Rel. Exp. (%) |
|---|---|---|---|
| | Ag5036, Run | | Ag5036, Run |
| Tissue Name | 223740995 | Tissue Name | 223740995 |
| Secondary Th1 act | 42.9 | HUVEC IL-1beta | 27.2 |
| Secondary Th2 act | 54.3 | HUVEC IFN gamma | 42.0 |
| Secondary Tr1 act | 35.4 | HUVEC TNF alpha + IFN | 21.8 |
| | | gamma | |
| Secondary Th1 rest | 21.2 | HUVEC TNF alpha + IL4 | 31.9 |
| Secondary Th2 rest | 35.6 | HUVEC IL-11 | 32.1 |
| Secondary Tr1 rest | 20.7 | Lung Microvascular EC none | 72.2 |
| Primary Th1 act | 13.3 | Lung Microvascular EC | 36.6 |
| | | TNF alpha + IL-1beta | |
| Primary Th2 act | 34.6 | Microvascular Dermal EC | 54.7 |
| | | none | |
| Primary Tr1 act | 37.1 | Microsvasular Dermal EC | 26.6 |
| | | TNF alpha + IL-1beta | |
| Primary Th1 rest | 19.5 | Bronchial epithelium | 25.5 |
| | | TNF alpha + IL1beta | |
| Primary Th2 rest | 23.0 | Small airway epithelium none | 14.2 |
| Primary Tr1 rest | 40.1 | Small airway epithelium | 26.2 |
| | | TNF alpha + IL-1beta | |
| CD45RA CD4 | 34.6 | Coronery artery SMC rest | 16.2 |
| lymphocyte act | | | |
| CD45RO CD4 | 51.8 | Coronery artery SMC | 19.3 |
| lymphocyte act | | TNF alpha + IL-1beta | |
| CD8 lymphocyte act | 28.3 | Astrocytes rest | 14.4 |
| Secondary CD8 | 31.0 | Astrocytes TNF alpha + IL- | 12.9 |
| lymphocyte rest | | 1beta | |
| Secondary CD8 | 17.3 | KU-812 (Basophil) rest | 50.0 |
| lymphocyte act | | | |
| CD4 lymphocyte none | 16.4 | KU-812 (Basophil) | 67.4 |
| | | PMA/ionomycin | |
| 2ry Th1/Th2/Tr1_anti- | 39.0 | CCD1106 (Keratinocytes) | 31.4 |
| CD95 CH11 | | none | |
| LAK cells rest | 32.5 | CCD1106 (Keratinocytes) | 53.2 |
| | | TNF alpha + IL-1beta | |
| LAK cells IL-2 | 38.7 | Liver cirrhosis | 10.4 |
| LAK cells IL-2 + IL-12 | 11.1 | NCI-H292 none | 44.4 |
| LAK cells IL-2 + IFN | 13.2 | NCI-H292 IL-4 | 69.7 |
| gamma | | | |
| LAK cells IL-2 + IL-18 | 21.0 | NCI-H292 IL-9 | 66.9 |
| LAK cells | 11.3 | NCI-H292 IL-13 | 57.0 |
| PMA/ionomycin | | | |
| NK Cells IL-2 rest | 49.3 | NCI-H292 IFN gamma | 49.3 |
| Two Way MLR 3 day | 27.9 | HPAEC none | 29.7 |
| Two Way MLR 5 day | 22.1 | HPAEC TNF alpha + IL-1 | 25.2 |
| | | beta | |
| Two Way MLR 7 day | 28.7 | Lung fibroblast none | 42.3 |
| PBMC rest | 20.6 | Lung fibroblast TNF alpha + IL-1 | 28.3 |
| | | beta | |
| PBMC PWM | 26.1 | Lung fibroblast IL-4 | 25.3 |
| PBMC PHA-L | 34.9 | Lung fibroblast IL-9 | 31.0 |
| Ramos (B cell) none | 19.8 | Lung fibroblast IL-13 | 22.1 |
| Ramos (B cell) ionomycin | 35.1 | Lung fibroblast IFN gamma | 40.1 |
| B lymphocytes PWM | 21.9 | Dermal fibroblast CCD1070 | 19.6 |
| | | rest | |
| B lymphocytes CD40L | 51.8 | Dermal fibroblast CCD1070 | 65.1 |
| and IL-4 | | TNF alpha | |
| EOL-1 dbcAMP | 19.1 | Dermal fibroblast CCD1070 | 12.1 |
| | | IL-1beta | |
| EOL-1 dbcAMP | 11.2 | Dermal fibroblast IFN gamma | 17.1 |
| PMA/ionomycin | | | |
| Dendritic cells none | 27.5 | Dermal fibroblast IL-4 | 22.1 |
| Dendritic cells LPS | 25.5 | Dermal fibroblasts rest | 26.8 |
| Dendritic cells anti-CD40 | 26.4 | Neutrophils TNFa + LPS | 16.5 |
| Monocytes rest | 53.2 | Neutrophils rest | 100.0 |
| Monocytes LPS | 31.4 | Colon | 6.5 |
| Macrophages rest | 27.2 | Lung | 22.4 |
| Macrophages LPS | 16.5 | Thymus | 13.5 |
| HUVEC none | 17.1 | Kidney | 25.2 |
| HUVEC starved | 38.4 | | |

General_screening_panel_v1.5 Summary: Ag5036 Highest expression of this gene is seen in an ovarian cancer cell line (CT=27). This gene is widely expressed in this panel, with moderate expression seen in brain, colon, gastric, lung, breast, ovarian, and melanoma cancer cell lines. This gene encodes a protein with homology to flotillin-2, an integral membrane protein of the plasmalemmal microdomains involved in vesicular trafficking and signal transduction. Cho has suggested that this molecule is involved in cell adhesion (Genomics 27: 251-258, 1995.). Thus, based on this expression profile and the homology of this gene to flotillin, this protein product may be involved in cell survival and/or proliferation. Modulation of this gene product may be useful in the treatment of cancer.

Among tissues with metabolic function, this gene is expressed at moderate levels in pituitary, adipose, adrenal gland, pancreas, thyroid, and adult and fetal skeletal muscle, heart, and liver. Flotillin-2 may play a role in the glucose uptake pathway (Baumann, Nature 2000 Sep 14;407(6801):202-7). This widespread expression among these metabolic tissues and the homology to flotillin suggest that this gene product may play a role in normal neuroendocrine and metabolic function and that disregulated expression of this gene may contribute to neuroendocrine disorders or metabolic diseases, such as obesity and diabetes.

In addition, this gene is expressed at much higher levels in fetal liver tissue (CT=28) when compared to expression in the adult counterpart (CT=31). Thus, expression of this gene may be used to differentiate between the fetal and adult source of this tissue.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag5036-Highest expression of this gene is seen in neutrophils (CT=28.2). This gene is also expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

S. CG140843-01: Integrin Beta-5 Precursor Protein-Like Protein.

Expression of gene CG 140843-01 was assessed using the primer-probe set Ag7404, described in Table SA. Results of the RTQ-PCR runs are shown in Table SB.Table SA. Probe Name Ag7404

TABLE SA

Probe Name Ag7404

| | | | | |
|---|---|---|---|---|
| Primers | Sequences | Length | Start Position | SEQ ID No |

| | | | | | |
|---|---|---|---|---|---|
| Forward | 5′-ctgcatggggaggtcaa-3′ | 17 | 852 | 312 | |
| Probe | | TET-5′- | 26 | 873 | 313 |
| | aagtaccaacacccactgacgctctc | | | | |
| | | -3′-TAMRA | | | |
| Reverse | 5′-gctggggcactcaaagact-3′ | 19 | 907 | 314 | |

TABLE SB
General_screening_panel_v1.6

| | Rel. Exp. (%) | | Rel. Exp. (%) |
|---|---|---|---|
| | Ag7404, Run | | Ag7404, Run |
| Tissue Name | 306066735 | Tissue Name | 306066735 |
| Adipose | 6.4 | Renal ca. TK-10 | 38.2 |
| Melanoma* Hs688(A).T | 21.6 | Bladder | 0.0 |
| Melanoma* Hs688(B).T | 14.2 | Gastric ca. (liver met.) | 34.2 |
| | | NCI-N87 | |
| Melanoma* M14 | 9.5 | Gastric ca. KATO III | 16.6 |
| Melanoma* LOXIMVI | 2.9 | Colon ca. SW-948 | 0.0 |
| Melanoma* SK-MEL-5 | 9.3 | Colon ca. SW480 | 100.0 |
| Squamous cell carcinoma | 2.9 | Colon ca.* (SW480 met) | 11.0 |
| SCC-4 | | SW620 | |
| Testis Pool | 5.1 | Colon ca. HT29 | 17.7 |
| Prostate ca.* (bone met) | 8.2 | Colon ca. HCT-116 | 18.2 |
| PC-3 | | | |
| Prostate Pool | 3.4 | Colon ca. CaCo-2 | 16.4 |
| Placenta | 0.0 | Colon cancer tissue | 7.6 |
| Uterus Pool | 10.2 | Colon ca. SW1116 | 0.0 |
| Ovarian ca. OVCAR-3 | 50.0 | Colon ca. Colo-205 | 4.3 |
| Ovarian ca. SK-OV-3 | 27.4 | Colon ca. SW-48 | 0.0 |
| Ovarian ca. OVCAR-4 | 0.0 | Colon Pool | 26.1 |
| Ovarian ca. OVCAR-5 | 40.1 | Small Intestine Pool | 11.3 |
| Ovarian ca. IGROV-1 | 4.2 | Stomach Pool | 18.7 |
| Ovarian ca. OVCAR-8 | 6.5 | Bone Marrow Pool | 4.5 |
| Ovary | 13.3 | Fetal Heart | 3.3 |
| Breast ca. MCF-7 | 24.1 | Heart Pool | 10.3 |
| Breast ca. MDA-MB-231 | 46.3 | Lymph Node Pool | 20.4 |
| Breast ca. BT 549 | 10.6 | Fetal Skeletal Muscle | 0.0 |
| Breast ca. T47D | 7.4 | Skeletal Muscle Pool | 0.0 |
| Breast ca. MDA-N | 5.6 | Spleen Pool | 15.5 |
| Breast Pool | 23.2 | Thymus Pool | 6.7 |
| Trachea | 9.0 | CNS cancer (glio/astro) | 10.2 |
| | | U87-MG | |
| Lung | 9.4 | CNS cancer (glio/astro) | 18.8 |
| | | U-118-MG | |
| Fetal Lung | 11.4 | CNS cancer (neuro; met) | 30.8 |
| | | SK-N-AS | |
| Lung ca. NCI-N417 | 0.0 | CNS cancer (astro) SF- | 17.8 |
| | | 539 | |
| Lung ca. LX-1 | 21.3 | CNS cancer (astro) SNB- | 43.8 |
| | | 75 | |
| Lung ca. NCI-H146 | 0.0 | CNS cancer (glio) SNB- | 6.8 |
| | | 19 | |
| Lung ca. SHP-77 | 0.0 | CNS cancer (glio) SF-295 | 20.7 |
| Lung ca. A549 | 35.4 | Brain (Amygdala) Pool | 0.0 |
| Lung ca. NCI-H526 | 0.0 | Brain (cerebellum) | 5.6 |
| Lung ca. NCI-H23 | 2.8 | Brain (fetal) | 0.0 |
| Lung ca. NCI-H460 | 10.7 | Brain (Hippocampus) | 7.3 |
| | | Pool | |
| Lung ca. HOP-62 | 12.8 | Cerebral Cortex Pool | 3.2 |
| Lung ca. NCI-H522 | 7.1 | Brain (Substantia nigra) | 7.1 |
| | | Pool | |
| Liver | 0.0 | Brain (Thalamus) Pool | 3.3 |
| Fetal Liver | 0.0 | Brain (whole) | 3.4 |
| Liver ca. HepG2 | 19.9 | Spinal Cord Pool | 13.4 |
| Kidney Pool | 9.0 | Adrenal Gland | 6.6 |
| Fetal Kidney | 12.2 | Pituitary gland Pool | 0.0 |
| Renal ca. 786-0 | 16.5 | Salivary Gland | 0.0 |
| Renal ca. A498 | 3.6 | Thyroid (female) | 0.0 |
| Renal ca. ACHN | 13.4 | Pancreatic ca. CAPAN2 | 35.6 |
| Renal ca. UO-31 | 19.6 | Pancreas Pool | 4.2 |

CNS_neurodegeneration_v1.0 Summary: Ag7404 Expression of this gene is low/undetectable in all samples on this panel (CTs>35).

General_screening_panel_v1.6 Summary: Ag7404 Expression of this gene is restricted to a sample derived from a colon cancer cell line (CT=34.8). Thus, expression of this gene could be used to differentiate between this sample and other samples on this panel and as a marker to detect the presence of colon cancer. Furthermore, therapeutic modulation of the expression or function of this gene may be effective in the treatment of colon cancer.

T. CG141540-01: IL1 Receptor-Type-2-Like Protein

Expression of gene CG141540-01 was assessed using the primer-probe sets Ag5237 and Ag5236, described in Tables TA and TB. Results of the RTQ-PCR runs are shown in Tables TC, TD and TE.

TABLE TA

Probe Name Ag5237

| | | | | |
|---|---|---|---|---|
| Primers | Sequences | Length | Start Position | SEQ ID No |

| | | | | | |
|---|---|---|---|---|---|
| Forward | 5′-agatggtctgactgtgctatg-3′ | 21 | 1143 | 315 | |
| Probe | | TET-5′- | 29 | 1167 | 316 |
| | tcatcatcaagactttcaatcctatccc | | | | |
| | | a-3′-TAMRA | | | |
| Reverse | 5′-gaattatttcattccatttatttc-3′ | 24 | 1199 | 317 | |

TABLE TB

Probe Name Ag5236

| | | | | |
|---|---|---|---|---|
| Primers | Sequences | Length | Start Position | SEQ ID No |

| | | | | | |
|---|---|---|---|---|---|
| Forward | 5′-acgcatcaagaggtcaagact-3′ | 21 | 744 | 318 | |
| Probe | | TET-5′- | 22 | 794 | 319 |
| | ccggcacacccttaaccaccat-3′-TAMRA | | | | |
| Reverse | 5′-gtgtcattggccgtcca-3′ | 17 | 823 | 320 | |

TABLE TC
A1_comprehensive panel_v1.0

| | Rel. Exp. (%) | | Rel. Exp. (%) |
|---|---|---|---|
| | Ag5236, | | Ag5236, |
| Tissue Name | Run 229545061 | Tissue Name | Run 229545061 |
| 110967 COPD-F | 0.0 | 112427 Match Control | 0.0 |
| | | Psoriasis-F | |
| 110980 COPD-F | 0.0 | 112418 Psoriasis-M | 0.0 |
| 110968 COPD-M | 1.4 | 112723 Match Control | 0.0 |
| | | Psoriasis-M | |
| 110977 COPD-M | 0.0 | 112419 Psoriasis-M | 0.0 |
| 110989 Emphysema-F | 0.0 | 112424 Match Control | 1.6 |
| | | Psoriasis-M | |
| 110992 Emphysema-F | 2.7 | 112420 Psoriasis-M | 3.8 |
| 110993 Emphysema-F | 1.8 | 112425 Match Control | 0.0 |
| | | Psoriasis-M | |
| 110994 Emphysema-F | 0.0 | 104689 (MF) OA Bone- | 3.4 |
| | | Backus | |
| 110995 Emphysema-F | 11.4 | 104690 (MF) Adj | 1.7 |
| | | “Normal” Bone-Backus | |
| 110996 Emphysema-F | 6.1 | 104691 (MF) OA | 1.7 |
| | | Synovium-Backus | |
| 110997 Asthma-M | 4.6 | 104692 (BA) OA | 0.0 |
| | | Cartilage-Backus | |
| 111001 Asthma-F | 0.0 | 104694 (BA) OA Bone- | 1.8 |
| | | Backus | |
| 111002 Asthma-F | 0.0 | 104695 (BA) Adj | 0.9 |
| | | “Normal” Bone-Backus | |
| 111003 Atopic Asthma-F | 0.7 | 104696 (BA) OA | 0.0 |
| | | Synovium-Backus | |
| 111004 Atopic Asthma-F | 0.0 | 104700 (SS) OA Bone- | 4.4 |
| | | Backus | |
| 111005 Atopic Asthma-F | 0.0 | 104701 (SS) Adj | 3.0 |
| | | “Normal” Bone-Backus | |
| 111006 Atopic Asthma-F | 0.0 | 104702 (SS) OA | 1.5 |
| | | Synovium-Backus | |
| 111417 Allergy-M | 0.0 | 117093 OA Cartilage | 1.4 |
| | | Rep7 | |
| 112347 Allergy-M | 0.0 | 112672 OA Bone5 | 0.0 |
| 112349 Normal Lung-F | 0.0 | 112673 OA Synovium5 | 0.0 |
| 112357 Normal Lung-F | 0.0 | 112674 OA Synovial | 1.2 |
| | | Fluid cells5 | |
| 112354 Normal Lung-M | 0.0 | 117100 OA Cartilage | 0.0 |
| | | Rep14 | |
| 112374 Crohns-F | 0.0 | 112756 OA Bone9 | 0.0 |
| 112389 Match Control | 6.1 | 112757 OA Synovium9 | 0.7 |
| Crohns-F | | | |
| 112375 Crohns-F | 0.0 | 112758 OA Synovial | 1.5 |
| | | Fluid Cells9 | |
| 112732 Match Control | 30.8 | 117125 RA Cartilage | 0.0 |
| Crohns-F | | Rep2 | |
| 112725 Crohns-M | 0.0 | 113492 Bone2 RA | 0.9 |
| 112387 Match Control | 1.5 | 113493 Synovium2 RA | 0.0 |
| Crohns-M | | | |
| 112378 Crohns-M | 0.0 | 113494 Syn Fluid Cells | 2.0 |
| | | RA | |
| 112390 Match Control | 0.0 | 113499 Cartilage4 RA | 2.5 |
| Crohns-M | | | |
| 112726 Crohns-M | 1.1 | 113500 Bone4 RA | 2.2 |
| 112731 Match Control | 1.7 | 113501 Synovium4 RA | 1.9 |
| Crohns-M | | | |
| 112380 Ulcer Col-F | 0.0 | 113502 Syn Fluid Cells4 | 0.0 |
| | | RA | |
| 112734 Match Control | 100.0 | 113495 Cartilage3 RA | 4.5 |
| Ulcer Col-F | | | |
| 112384 Ulcer Col-F | 1.8 | 113496 Bone3 RA | 5.3 |
| 112737 Match Control | 0.6 | 113497 Synovium3 RA | 2.3 |
| Ulcer Col-F | | | |
| 112386 Ulcer Col-F | 1.2 | 113498 Syn Fluid Cells3 | 2.0 |
| | | RA | |
| 112738 Match Control | 4.6 | 117106 Normal Cartilage | 0.0 |
| Ulcer Col-F | | Rep20 | |
| 112381 Ulcer Col-M | 0.0 | 113663 Bone3 Normal | 0.0 |
| 112735 Match Control | 0.0 | 113664 Synovium3 | 0.0 |
| Ulcer Col-M | | Normal | |
| 112382 Ulcer Col-M | 7.9 | 113665 Syn Fluid Cells3 | 0.0 |
| | | Normal | |
| 112394 Match Control | 0.0 | 117107 Normal Cartilage | 0.0 |
| Ulcer Col-M | | Rep22 | |
| 112383 Ulcer Col-M | 1.9 | 113667 Bone4 Normal | 0.0 |
| 112736 Match Control | 0.0 | 113668 Synovium4 | 0.0 |
| Ulcer Col-M | | Normal | |
| 112423 Psoriasis-F | 2.3 | 113669 Syn Fluid Cells4 | 0.0 |
| | | Normal | |

TABLE TD
General_screening_panel_v1.5

| | Rel. Exp. (%) | | Rel. Exp. (%) |
|---|---|---|---|
| | Ag5236, Run | | Ag5236, Run |
| Tissue Name | 237228536 | Tissue Name | 237228536 |
| Adipose | 22.2 | Renal ca. TK-10 | 3.2 |
| Melanoma* Hs688(A).T | 0.0 | Bladder | 16.8 |
| Melanoma* Hs688(B).T | 0.0 | Gastric ca. (liver met.) | 11.4 |
| | | NCI-N87 | |
| Melanoma* M14 | 0.0 | Gastric ca. KATO III | 40.1 |
| Melanoma* LOXIMVI | 0.0 | Colon ca. SW-948 | 29.3 |
| Melanoma* SK-MEL-5 | 0.7 | Colon ca. SW480 | 0.0 |
| Squamous cell carcinoma | 8.2 | Colon ca.* (SW480 met) | 4.9 |
| SCC-4 | | SW620 | |
| Testis Pool | 1.4 | Colon ca. HT29 | 13.3 |
| Prostate ca.* (bone met) | 1.0 | Colon ca. HCT-116 | 0.0 |
| PC-3 | | | |
| Prostate Pool | 0.0 | Colon ca. CaCo-2 | 42.0 |
| Placenta | 13.4 | Colon cancer tissue | 35.1 |
| Uterus Pool | 3.5 | Colon ca. SW1116 | 0.0 |
| Ovarian ca. OVCAR-3 | 26.2 | Colon ca. Colo-205 | 67.4 |
| Ovarian ca. SK-OV-3 | 100.0 | Colon ca. SW-48 | 3.3 |
| Ovarian ca. OVCAR-4 | 80.7 | Colon Pool | 2.7 |
| Ovarian ca. OVCAR-5 | 1.3 | Small Intestine Pool | 2.9 |
| Ovarian ca. IGROV-1 | 25.3 | Stomach Pool | 2.0 |
| Ovarian ca. OVCAR-8 | 0.0 | Bone Marrow Pool | 5.0 |
| Ovary | 18.6 | Fetal Heart | 0.8 |
| Breast ca. MCF-7 | 0.0 | Heart Pool | 0.0 |
| Breast ca. MDA-MB-231 | 0.0 | Lymph Node Pool | 0.0 |
| Breast ca. BT 549 | 3.2 | Fetal Skeletal Muscle | 0.5 |
| Breast ca. T47D | 0.0 | Skeletal Muscle Pool | 0.0 |
| Breast ca. MDA-N | 0.0 | Spleen Pool | 59.5 |
| Breast Pool | 1.2 | Thymus Pool | 17.4 |
| Trachea | 12.6 | CNS cancer (glio/astro) | 6.8 |
| | | U87-MG | |
| Lung | 0.0 | CNS cancer (glio/astro) | 0.0 |
| | | U-118-MG | |
| Fetal Lung | 6.6 | CNS cancer (neuro; met) | 6.3 |
| | | SK-N-AS | |
| Lung ca. NCI-N417 | 0.0 | CNS cancer (astro) SF- | 1.1 |
| | | 539 | |
| Lung ca. LX-1 | 2.1 | CNS cancer (astro) SNB- | 18.7 |
| | | 75 | |
| Lung ca. NCI-H146 | 0.0 | CNS cancer (glio) SNB- | 85.3 |
| | | 19 | |
| Lung ca. SHP-77 | 0.0 | CNS cancer (glio) SF-295 | 3.4 |
| Lung ca. A549 | 8.0 | Brain (Amygdala) Pool | 2.1 |
| Lung ca. NCI-H526 | 0.0 | Brain (cerebellum) | 0.0 |
| Lung ca. NCI-H23 | 0.0 | Brain (fetal) | 3.4 |
| Lung ca. NCI-H460 | 3.4 | Brain (Hippocampus) | 0.0 |
| | | Pool | |
| Lung ca. HOP-62 | 2.7 | Cerebral Cortex Pool | 0.0 |
| Lung ca. NCI-H522 | 0.0 | Brain (Substantia nigra) | 0.0 |
| | | Pool | |
| Liver | 0.8 | Brain (Thalamus) Pool | 0.8 |
| Fetal Liver | 4.1 | Brain (whole) | 1.8 |
| Liver ca. HepG2 | 7.0 | Spinal Cord Pool | 0.0 |
| Kidney Pool | 2.8 | Adrenal Gland | 3.2 |
| Fetal Kidney | 0.0 | Pituitary gland Pool | 0.0 |
| Renal ca. 786-0 | 11.3 | Salivary Gland | 1.0 |
| Renal ca. A498 | 3.3 | Thyroid (female) | 0.8 |
| Renal ca. ACHN | 0.6 | Pancreatic ca. CAPAN2 | 0.0 |
| Renal ca. UO-31 | 0.0 | Pancreas Pool | 13.9 |

TABLE TE
Panel 4.1D

| | Rel. Exp. (%) |
|---|---|
| | Ag5236, Run |
| Tissue Name | 229788311 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 2.1 |
| CD45RO CD4 lymphocyte act | 1.1 |
| CD8 lymphocyte act | 0.0 |
| Secondary CD8 lymphocyte rest | 1.4 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 3.7 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 12.4 |
| NK Cells IL-2 rest | 0.0 |
| Two Way MLR 3 day | 2.2 |
| Two Way MLR 5 day | 1.1 |
| Two Way MLR 7 day | 0.0 |
| PBMC rest | 0.0 |
| PBMC PWM | 0.9 |
| PBMC PHA-L | 0.9 |
| Ramos (B cell) none | 0.0 |
| Ramos (B cell) ionomycin | 0.0 |
| B lymphocytes PWM | 4.0 |
| B lymphocytes CD40L and IL-4 | 1.6 |
| EOL-1 dbcAMP | 0.0 |
| EOL-1 dbcAMP PMA/ionomycin | 0.0 |
| Dendritic cells none | 10.3 |
| Dendritic cells LPS | 0.0 |
| Dendritic cells anti-CD40 | 4.7 |
| Monocytes rest | 0.0 |
| Monocytes LPS | 1.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 5.9 |
| Coronery artery SMC rest | 0.0 |
| Coronery artery SMC TNFalpha + IL-1beta | 0.0 |
| Astrocytes rest | 0.7 |
| Astrocytes TNFalpha + IL-1beta | 0.0 |
| KU-812 (Basophil) rest | 0.9 |
| KU-812 (Basophil) PMA/ionomycin | 0.0 |
| CCD1106 (Keratinocytes) none | 2.5 |
| CCD1106 (Keratinocytes) TNFalpha + IL-1beta | 7.4 |
| Liver cirrhosis | 2.5 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 85.3 |
| Neutrophils rest | 100.0 |
| Colon | 0.0 |
| Lung | 1.0 |
| Thymus | 2.8 |
| Kidney | 0.0 |

AI_comprehensive panel_v1.0 Summary: Ag5236 Expression of this gene is limited to a normal tissue sample adjacent to Crohn's and normal tissue sample adjacent to ulcerative colitis (CTs=32-34). Thus, expression of this gene could be used to differentiate between these samples and other samples on this panel. General_screening_panel_v1.5 Summary: Ag5236 Highest expression of this gene is seen in an ovarian cancer cell line (CT=32). Low but significant levels of expression are also seen in clusters of cell lines derived from brain, ovarian, colon and gastric cancers. Thus, this gene product may be involved in these cancers. Low levels of expression are also seen in adipose and pancreas suggesting a role for this gene product in the pathogenesis of metabolic disorders including obesity and diabetes.

Panel 4.1D Summary: Ag5236 This gene is expressed exclusively in neutrophils. Thus, expression of this gene could be used to differentiate between these samples and other samples on this panel and as a marker of neutrophils.

U. CG141580-01: KIAA 1467 Protein-Like Protein.

Expression of gene CG141580-01 was assessed using the primer-probe set Ag7248, described in Table UA. Results of the RTQ-PCR runs are shown in Tables UB and UC.

TABLE UA

Probe Name Ag7248

| | | | | |
|---|---|---|---|---|
| Primers | Sequences | Length | Start Position | SEQ ID No |

| | | | | |
|---|---|---|---|---|
| Forward | 5′- | 29 | 2255 | 321 |
| | gtctatgactaggaaacattttgttgtac | | | |
| | -3′ | | | |
| Probe | TET-5′- | 30 | 2289 | 322 |
| | | ccacaacactaaaatatacacacacacag | | |
| | c-3′-TAMRA | | | |
| Reverse | 5′- | 27 | 2320 | 323 |
| | cttaggacatacctqgaaaataacttc- | | | |
| | 3′ | | | |

TABLE UB
CNS_neurodegeneration_v1.0

| | Rel. Exp. (%) |
|---|---|
| | Ag7248, Run |
| Tissue Name | 296423801 |
| AD 1 Hippo | 6.3 |
| AD 2 Hippo | 14.1 |
| AD 3 Hippo | 2.5 |
| AD 4 Hippo | 1.9 |
| AD 5 Hippo | 100.0 |
| AD 6 Hippo | 27.2 |
| Control 2 Hippo | 13.3 |
| Control 4 Hippo | 1.8 |
| Control (Path) 3 Hippo | 2.5 |
| AD 1 Temporal Ctx | 3.5 |
| AD 2 Temporal Ctx | 8.2 |
| AD 3 Temporal Ctx | 1.7 |
| AD 4 Temporal Ctx | 17.8 |
| AD 5 Inf Temporal Ctx | 60.7 |
| AD 5 Sup Temporal Ctx | 20.7 |
| AD 6 Inf Temporal Ctx | 25.5 |
| AD 6 Sup Temporal Ctx | 30.8 |
| Control 1 Temporal Ctx | 1.3 |
| Control 2 Temporal Ctx | 24.5 |
| Control 3 Temporal Ctx | 5.5 |
| Control 3 Temporal Ctx | 2.8 |
| Control (Path) 1 Temporal Ctx | 37.9 |
| Control (Path) 2 Temporal Ctx | 21.0 |
| Control (Path) 3 Temporal Ctx | 1.2 |
| Control (Path) 4 Temporal Ctx | 12.9 |
| AD 1 Occipital Ctx | 6.1 |
| AD 2 Occipital Ctx (Missing) | 0.0 |
| AD 3 Occipital Ctx | 1.2 |
| AD 4 Occipital Ctx | 9.9 |
| AD 5 Occipital Ctx | 25.5 |
| AD 6 Occipital Ctx | 23.5 |
| Control 1 Occipital Ctx | 0.9 |
| Control 2 Occipital Ctx | 43.8 |
| Control 3 Occipital Ctx | 7.6 |
| Control 4 Occipital Ctx | 1.2 |
| Control (Path) 1 Occipital Ctx | 66.4 |
| Control (Path) 2 Occipital Ctx | 7.6 |
| Control (Path) 3 Occipital Ctx | 0.5 |
| Control (Path) 4 Occipital Ctx | 6.7 |
| Control 1 Parietal Ctx | 1.8 |
| Control 2 Parietal Ctx | 16.3 |
| Control 3 Parietal Ctx | 14.4 |
| Control (Path) 1 Parietal Ctx | 67.8 |
| Control (Path) 2 Parietal Ctx | 14.6 |
| Control (Path) 3 Parietal Ctx | 1.7 |
| Control (Path) 4 Parietal Ctx | 35.6 |

TABLE UC
Panel 4.1D

| | Rel. Exp. (%) |
|---|---|
| | Ag7248, Run |
| Tissue Name | 296417628 |
| Secondary Th1 act | 53.6 |
| Secondary Th2 act | 50.0 |
| Secondary Tr1 act | 16.8 |
| Secondary Th1 rest | 1.7 |
| Secondary Th2 rest | 2.0 |
| Secondary Tr1 rest | 6.7 |
| Primary Th1 act | 5.6 |
| Primary Th2 act | 33.7 |
| Primary Tr1 act | 27.7 |
| Primary Th1 rest | 1.2 |
| Primary Th2 rest | 2.1 |
| Primary Tr1 rest | 0.0 |
| CD45RA CD4 lymphocyte act | 36.9 |
| CD45RO CD4 lymphocyte act | 55.5 |
| CD8 lymphocyte act | 11.1 |
| Secondary CD8 lymphocyte rest | 5.2 |
| Secondary CD8 lymphocyte act | 4.5 |
| CD4 lymphocyte none | 4.3 |
| 2ry Th1/Th2/Tr1_anti-CD95 CH11 | 4.7 |
| LAK cells rest | 20.4 |
| LAK cells IL-2 | 4.6 |
| LAK cells IL-2 + IL-12 | 1.6 |
| LAK cells IL-2 + IFN gamma | 9.1 |
| LAK cells IL-2 + IL-18 | 3.9 |
| LAK cells PMA/ionomycin | 37.1 |
| NK Cells IL-2 rest | 12.8 |
| Two Way MLR 3 day | 27.9 |
| Two Way MLR 5 day | 3.3 |
| Two Way MLR 7 day | 8.8 |
| PBMC rest | 5.4 |
| PBMC PWM | 11.4 |
| PBMC PHA-L | 12.4 |
| Ramos (B cell) none | 20.6 |
| Ramos (B cell) ionomycin | 53.2 |
| B lymphocytes PWM | 4.9 |
| B lymphocytes CD40L and IL-4 | 17.9 |
| EOL-1 dbcAMP | 38.4 |
| EOL-1 dbcAMP PMA/ionomycin | 37.1 |
| Dendritic cells none | 24.0 |
| Dendritic cells LPS | 3.4 |
| Dendritic cells anti-CD40 | 2.6 |
| Monocytes rest | 8.8 |
| Monocytes LPS | 41.5 |
| Macrophages rest | 11.6 |
| Macrophages LPS | 3.0 |
| HUVEC none | 22.1 |
| HUVEC starved | 35.8 |
| HUVEC IL-1beta | 36.1 |
| HUVEC IFN gamma | 37.6 |
| HUVEC TNF alpha + IFN gamma | 7.1 |
| HUVEC TNF alpha + IL4 | 14.3 |
| HUVEC IL-11 | 11.3 |
| Lung Microvascular EC none | 100.0 |
| Lung Microvascular EC TNFalpha + IL-1beta | 12.3 |
| Microvascular Dermal EC none | 18.2 |
| Microsvasular Dermal EC TNFalpha + IL-1beta | 8.0 |
| Bronchial epithelium TNFalpha + IL1beta | 11.0 |
| Small airway epithelium none | 11.7 |
| Small airway epithelium TNFalpha + IL-1beta | 27.9 |
| Coronery artery SMC rest | 34.9 |
| Coronery artery SMC TNFalpha + IL-1beta | 41.5 |
| Astrocytes rest | 10.9 |
| Astrocytes TNFalpha + IL-1beta | 10.0 |
| KU-812 (Basophil) rest | 63.3 |
| KU-812 (Basophil) PMA/ionomycin | 81.2 |
| CCD1106 (Keratinocytes) none | 28.1 |
| CCD1106 (Keratinocytes) TNFalpha + IL-1beta | 3.9 |
| Liver cirrhosis | 5.3 |
| NCI-H292 none | 13.5 |
| NCI-H292 IL-4 | 20.6 |
| NCI-H292 IL-9 | 21.6 |
| NCI-H292 IL-13 | 28.7 |
| NCI-H292 IFN gamma | 14.5 |
| HPAEC none | 10.8 |
| HPAEC TNF alpha + IL-1beta | 35.8 |
| Lung fibroblast none | 44.4 |
| Lung fibroblast TNF alpha + IL-1 beta | 45.4 |
| Lung fibroblast IL-4 | 12.5 |
| Lung fibroblast IL-9 | 14.6 |
| Lung fibroblast IL-13 | 11.3 |
| Lung fibroblast IFN gamma | 37.6 |
| Dermal fibroblast CCD1070 rest | 24.8 |
| Dermal fibroblast CCD1070 TNF alpha | 33.0 |
| Dermal fibroblast CCD1070 IL-1 beta | 21.0 |
| Dermal fibroblast IFN gamma | 16.2 |
| Dermal fibroblast IL-4 | 56.6 |
| Dermal Fibroblasts rest | 22.7 |
| Neutrophils TNFa + LPS | 1.5 |
| Neutrophils rest | 2.9 |
| Colon | 5.4 |
| Lung | 1.1 |
| Thymus | 5.6 |
| Kidney | 50.0 |

CNS_neurodegeneration_v1.0 Summary: Ag7248 This panel confirms the expression of this gene at low levels in the brains of an independent group of individuals. However, no differential expression of this gene was detected between Alzheimer's diseased postmortem brains and those of non-demented controls in this experiment. Low levels of expression of this gene in brain regions suggests that this gene may play a role in central nervous system disorders such as Alzhcimer's disease, Parkinson's disease, epilepsy, multiple sclerosis, schizophrenia and depression.

Panel 4.1D Summary: Ag7248 Highest expression of this gene is detected in lung microvascular endothelial cells (CT=32). Expression of this gene is down-regulated on activation of these endothelial cells by cytokines. Thus, this gene may be play a role in the maintenance of the integrity of the microvasculature. Therefore, therapeutics designed for this putative protein could be beneficial for the treatment of diseases associated with damaged microvasculature including inflammatory diseases of lung, such as asthma, allergy, and chronic obstructive pulmonary diseases.

In addition, low to moderate levels of expression of this gene is also seen in lung and dermal fibroblasts, keratinocytes, basophils, coronery artery SMC, cytokine activated small airway epithelium, dermal microvascular EC, HUVEC, cytokine activated HPAEC, activated monocytes, eosinophils, Rainos B cells, two way MLR, activated LAK cells, and various types of activated T cells. Therefore, therapeutic modulation of this gene may be useful in the treatment of inflammatory and autoimmune diseases such as asthma, allergies, inflammatory bowel disease, lupus erythematosus, psoriasis, rheumatoid arthritis, and osteoarthritis.

V. CG141643-01:Riken 2010001CC9 Protein-Like Protein.

Expression of gene CG141643-01 was assessed using the primer-probe set Ag5057, described in Table VA. Results of the RTQ-PCR runs are shown in Tables VB, VC and VD.

TABLE VA

Probe Name Ag5057

| | | | | |
|---|---|---|---|---|
| | | | Start | |
| Primers | Sequences | Length | Position | SEQ ID No |

| | | | | |
|---|---|---|---|---|
| Forward | 5′-gcgtccaggaaccttcttc-3′ | 19 | 355 | 324 |
| Probe | TET-5′-actgggtcctgctggcactagctct-3′-TAMRA | 25 | 386 | 325 |
| Reverse | 5′-caacggacaagagcaggtt-3′ | 19 | 415 | 326 |

TABLE VB
AI_comprehensive panel_v1.0

| | Rel. Exp. (%) |
|---|---|
| | Ag5057, Run |
| Tissue Name | 219965745 |
| 110967 COPD-F | 7.0 |
| 110980 COPD-F | 3.6 |
| 110968 COPD-M | 12.7 |
| 110977 COPD-M | 0.0 |
| 110989 Emphysema-F | 30.1 |
| 110992 Emphysema-F | 22.2 |
| 110993 Emphysema-F | 11.1 |
| 110994 Emphysema-F | 5.8 |
| 110995 Emphysema-F | 98.6 |
| 110996 Emphysema-F | 13.6 |
| 110997 Asthma-M | 19.9 |
| 111001 Asthma-F | 8.2 |
| 111002 Asthma-F | 21.3 |
| 111003 Atopic Asthma-F | 25.5 |
| 111004 Atopic Asthma-F | 87.1 |
| 111005 Atopic Asthma-F | 32.1 |
| 111006 Atopic Asthma-F | 14.1 |
| 111417 Allergy-M | 33.2 |
| 112347 Allergy-M | 10.7 |
| 112349 Normal Lung-F | 25.3 |
| 112357 Normal Lung-F | 30.1 |
| 112354 Normal Lung-M | 17.2 |
| 112374 Crohns-F | 10.7 |
| 112389 Match Control Crohns-F | 9.5 |
| 112375 Crohns-F | 14.2 |
| 112732 Match Control Crohns-F | 54.7 |
| 112725 Crohns-M | 11.8 |
| 112387 Match Control Crohns-M | 10.3 |
| 112378 Crohns-M | 7.4 |
| 112390 Match Control Crohns-M | 42.6 |
| 112726 Crohns-M | 32.3 |
| 112731 Match Control Crohns-M | 42.3 |
| 112380 Ulcer Col-F | 12.6 |
| 112734 Match Control Ulcer Col-F | 85.3 |
| 112384 Ulcer Col-F | 50.3 |
| 112737 Match Control Ulcer Col-F | 16.7 |
| 112386 Ulcer Col-F | 2.6 |
| 112738 Match Control Ulcer Col-F | 100.0 |
| 112381 Ulcer Col-M | 3.5 |
| 112735 Match Control Ulcer Col-M | 24.1 |
| 112382 Ulcer Col-M | 20.0 |
| 112394 Match Control Ulcer Col-M | 2.7 |
| 112383 Ulcer Col-M | 23.2 |
| 112736 Match Control Ulcer Col-M | 11.3 |
| 112423 Psoriasis-F | 11.5 |
| 112427 Match Control Psoriasis-F | 80.7 |
| 112418 Psoriasis-M | 7.0 |
| 112723 Match Control Psoriasis-M | 5.0 |
| 112419 Psoriasis-M | 8.8 |
| 112424 Match Control Psoriasis-M | 25.0 |
| 112420 Psoriasis-M | 70.7 |
| 112425 Match Control Psoriasis-M | 48.3 |
| 104689 (MF) OA Bone-Backus | 7.0 |
| 104690 (MF) Adj “Normal” Bone-Backus | 8.1 |
| 104691 (MF) OA Synovium-Backus | 10.7 |
| 104692 (BA) OA Cartilage-Backus | 10.4 |
| 104694 (BA) OA Bone-Backus | 7.7 |
| 104695 (BA) Adj “Normal” Bone-Backus | 8.8 |
| 104696 (BA) OA Synovium-Backus | 3.0 |
| 104700 (SS) OA Bone-Backus | 6.8 |
| 104701 (SS) Adj “Normal” Bone-Backus | 10.7 |
| 104702 (SS) OA Synovium-Backus | 9.6 |
| 117093 OA Cartilage Rep7 | 5.0 |
| 112672 OA Bone5 | 20.9 |
| 112673 OA Synovium5 | 6.4 |
| 112674 OA Synovial Fluid cells5 | 14.1 |
| 117100 OA Cartilage Rep14 | 8.0 |
| 112756 OA Bone9 | 23.0 |
| 112757 OA Synovium9 | 3.3 |
| 112758 OA Synovial Fluid Cells9 | 11.0 |
| 117125 RA Cartilage Rep2 | 2.9 |
| 113492 Bone2 RA | 27.5 |
| 113493 Synovium2 RA | 17.4 |
| 113494 Syn Fluid Cells RA | 38.4 |
| 113499 Cartilage4 RA | 49.3 |
| 113500 Bone4 RA | 51.8 |
| 113501 Synovium4 RA | 45.1 |
| 113502 Syn Fluid Cells4 RA | 34.4 |
| 113495 Cartilage3 RA | 13.8 |
| 113496 Bone3 RA | 23.8 |
| 113497 Synovium3 RA | 22.8 |
| 113498 Syn Fluid Cells3 RA | 24.5 |
| 117106 Normal Cartilage Rep20 | 6.3 |
| 113663 Bone3 Normal | 5.8 |
| 113664 Synovuim3 Normal | 7.5 |
| 113665 Syn Fluid Cells3 Normal | 7.1 |
| 117107 Normal Cartilage Rep22 | 2.7 |
| 113667 Bone4 Normal | 8.2 |
| 113668 Synovium4 Normal | 14.7 |
| 113669 Syn Fluid Cells4 Normal | 19.2 |

TABLE VC
General_screening_panel_v1.4

| | Rel. Exp. (%) |
|---|---|
| | Ag5057, Run |
| Tissue Name | 219514716 |
| Adipose | 0.5 |
| Melanoma* Hs688(A).T | 0.1 |
| Melanoma* Hs688(B).T | 0.1 |
| Melanoma* M14 | 0.4 |
| Melanoma* LOXIMVI | 0.2 |
| Melanoma* SK-MEL-5 | 0.0 |
| Squamous cell carcinoma SCC-4 | 15.8 |
| Testis Pool | 0.8 |
| Prostate ca.* (bone met) PC-3 | 0.5 |
| Prostate Pool | 2.1 |
| Placenta | 0.3 |
| Uterus Pool | 0.1 |
| Ovarian ca. OVCAR-3 | 1.7 |
| Ovarian ca. SK-OV-3 | 3.3 |
| Ovarian ca. OVCAR-4 | 22.1 |
| Ovarian ca. OVCAR-5 | 24.1 |
| Ovarian ca. IGROV-1 | 1.0 |
| Ovarian ca. OVCAR-8 | 0.4 |
| Ovary | 0.8 |
| Breast ca. MCF-7 | 17.3 |
| Breast ca. MDA-MB-231 | 0.5 |
| Breast ca. BT 549 | 0.5 |
| Breast ca. T47D | 51.1 |
| Breast ca. MDA-N | 0.4 |
| Breast Pool | 1.2 |
| Trachea | 4.7 |
| Lung | 0.9 |
| Fetal Lung | 1.6 |
| Lung ca. NCI-N417 | 0.2 |
| Lung ca. LX-1 | 30.4 |
| Lung ca. NCI-H146 | 9.7 |
| Lung ca. SHP-77 | 0.3 |
| Lung ca. A549 | 0.9 |
| Lung ca. NCI-H526 | 6.3 |
| Lung ca. NCI-H23 | 1.3 |
| Lung ca. NCI-H460 | 1.1 |
| Lung ca. HOP-62 | 0.7 |
| Lung ca. NCI-H522 | 0.9 |
| Liver | 0.7 |
| Fetal Liver | 1.4 |
| Liver ca. HepG2 | 1.0 |
| Kidney Pool | 0.9 |
| Fetal Kidney | 1.0 |
| Renal ca. 786-0 | 0.5 |
| Renal ca. A498 | 0.1 |
| Renal ca. ACHN | 0.5 |
| Renal ca. UO-31 | 0.5 |
| Renal ca. TK-10 | 1.6 |
| Bladder | 12.9 |
| Gastric ca. (liver met) NCI-N87 | 43.2 |
| Gastric ca. KATO III | 100.0 |
| Colon ca. SW-948 | 21.9 |
| Colon ca. SW480 | 1.2 |
| Colon ca.* (SW480 met) SW620 | 0.4 |
| Colon ca. HT29 | 24.3 |
| Colon ca. HCT-116 | 53.2 |
| Colon ca. CaCo-2 | 26.2 |
| Colon cancer tissue | 33.2 |
| Colon ca. SW1116 | 14.1 |
| Colon ca. Colo 205 | 29.5 |
| Colon ca. SW-48 | 25.3 |
| Colon Pool | 0.5 |
| Small Intestine Pool | 1.5 |
| Stomach Pool | 1.0 |
| Bone Marrow Pool | 0.1 |
| Fetal Heart | 0.1 |
| Heart Pool | 0.2 |
| Lymph Node Pool | 1.0 |
| Fetal Skeletal Muscle | 0.1 |
| Skeletal Muscle Pool | 0.1 |
| Spleen Pool | 0.4 |
| Thymus Pool | 1.8 |
| CNS cancer (glio/astro) U87-MG | 0.8 |
| CNS cancer (glio/astro) U-118-MG | 1.0 |
| CNS cancer (neuro, met) SK-N-AS | 0.5 |
| CNS cancer (astro) SF-539 | 0.5 |
| CNS cancer (astro) SNB-75 | 0.6 |
| CNS cancer (glio) SNB-19 | 0.9 |
| CNS cancer (glio) SF-295 | 2.9 |
| Brain (Amygdala) Pool | 0.2 |
| Brain (cerebellum) | 0.9 |
| Brain (fetal) | 0.8 |
| Brain (Hippocampus) Pool | 0.2 |
| Cerebral Cortex Pool | 0.2 |
| Brain (Substantia nigra) Pool | 0.2 |
| Brain (Thalamus) Pool | 0.3 |
| Brain (whole) | 0.2 |
| Spinal Cord Pool | 0.3 |
| Adrenal Gland | 0.8 |
| Pituitary gland Pool | 1.3 |
| Salivary Gland | 1.4 |
| Thyroid (female) | 2.1 |
| Pancreatic ca. CAPAN2 | 40.3 |
| Pancreas Pool | 3.3 |

TABLE VD
Panel 4.1D

| | Rel. Exp. (%) |
|---|---|
| | Ag5057, Run |
| Tissue Name | 220366655 |
| Secondary Th1 act | 14.0 |
| Secondary Th2 act | 15.8 |
| Secondary Tr1 act | 1.4 |
| Secondary Th1 rest | 3.8 |
| Secondary Th2 rest | 5.3 |
| Secondary Tr1 rest | 10.2 |
| Primary Th1 act | 2.3 |
| Primary Th2 act | 8.2 |
| Primary Tr1 act | 9.7 |
| Primary Th1 rest | 14.5 |
| Primary Th2 rest | 0.0 |
| Primary Tr1 rest | 8.3 |
| CD45RA CD4 lymphocyte act | 0.6 |
| CD45RO CD4 lymphocyte act | 17.3 |
| CD8 lymphocyte act | 17.3 |
| Secondary CD8 lymphocyte rest | 5.6 |
| Secondary CD8 lymphocyte act | 15.1 |
| CD4 lymphocyte none | 5.0 |
| 2ry Th1/Th2/Tr1_anti-CD95 CH11 | 7.3 |
| LAK cells rest | 15.8 |
| LAK cells IL-2 | 27.4 |
| LAK cells IL-2 + IL-12 | 7.7 |
| LAK cells IL-2 + IFN gamma | 9.6 |
| LAK cells IL-2 + IL-18 | 6.3 |
| LAK cells PMA/ionomycin | 6.0 |
| NK Cells IL-2 rest | 20.2 |
| Two Way MLR 3 day | 12.2 |
| Two Way MLR 5 day | 7.7 |
| Two Way MLR 7 day | 4.4 |
| PBMC rest | 2.4 |
| PBMC PWM | 11.2 |
| PBMC PHA-L | 9.5 |
| Ramos (B cell) none | 5.8 |
| Ramos (B cell) ionomycin | 0.0 |
| B lymphocytes PWM | 4.2 |
| B lymphocytes CD40L and IL-4 | 9.5 |
| EOL-1 dbcAMP | 5.0 |
| EOL-1 dbcAMP PMA/ionomycin | 0.0 |
| Dendritic cells none | 17.8 |
| Dendritic cells LPS | 2.3 |
| Dendritic cells anti-CD40 | 5.6 |
| Monocytes rest | 7.6 |
| Monocytes LPS | 13.2 |
| Macrophages rest | 23.7 |
| Macrophages LPS | 2.6 |
| HUVEC none | 9.3 |
| HUVEC starved | 13.8 |
| HUVEC IL-1beta | 3.8 |
| HUVEC IFN gamma | 9.7 |
| HUVEC TNF alpha + IFN gamma | 6.7 |
| HUVEC TNF alpha + IL4 | 2.4 |
| HUVEC IL-11 | 2.3 |
| Lung Microvascular EC none | 17.1 |
| Lung Microvascular EC TNFalpha + IL-1beta | 4.9 |
| Microvascular Dermal EC none | 0.0 |
| Microsvasular Dermal EC TNFalpha + IL-1beta | 4.4 |
| Bronchial epithelium TNFalpha + IL1beta | 24.3 |
| Small airway epithelium none | 19.9 |
| Small airway epithelium TNFalpha + IL-1beta | 100.0 |
| Coronery artery SMC rest | 0.9 |
| Coronery artery SMC TNFalpha + IL-1beta | 0.0 |
| Astrocytes rest | 4.2 |
| Astrocytes TNFalpha + IL-1beta | 7.1 |
| KU-812 (Basophil) rest | 0.0 |
| KU-812 (Basophil) PMA/ionomycin | 3.2 |
| CCD1106 (Keratinocytes) none | 53.2 |
| CCD1106 (Keratinocytes) TNFalpha + IL-1beta | 59.0 |
| Liver cirrhosis | 27.9 |
| NCI-H292 none | 3.5 |
| NCI-H292 IL-4 | 11.8 |
| NCI-H292 IL-9 | 3.7 |
| NCI-H292 IL-13 | 10.0 |
| NCI-H292 IFN gamma | 20.3 |
| HPAEC none | 3.2 |
| HPAEC TNFalpha + IL-1 beta | 9.4 |
| Lung fibroblast none | 2.5 |
| Lung fibroblast TNF alpha + IL-1 beta | 4.0 |
| Lung fibroblast IL-4 | 4.3 |
| Lung fibroblast IL-9 | 7.1 |
| Lung fibroblast IL-13 | 4.0 |
| Lung fibroblast IFN gamma | 2.7 |
| Dermal fibroblast CCD1070 rest | 4.5 |
| Dermal fibroblast CCD1070 TNF alpha | 8.8 |
| Dermal fibroblast CCD1070 IL-1 beta | 2.2 |
| Dermal fibroblast IFN gamma | 0.5 |
| Dermal fibroblast IL-4 | 2.4 |
| Dermal Fibroblasts rest | 2.8 |
| Neutrophils TNFa + LPS | 2.4 |
| Neutrophils rest | 2.3 |
| Colon | 39.2 |
| Lung | 16.5 |
| Thymus | 19.1 |
| Kidney | 44.8 |

AI_comprehensive panel_v1.0 Summary: Ag5057 Highest expression of this gene is detected in a matched control for ulcerative colitis (CT=30.2). This gene shows a ubiquitous expression with moderate to low levels of expression in normal and diseased lung (COPD, emphysema and asthma), normal and diseased colon (Crohn's and ulcerative colitis), psoriasis, bone, cartilage, synovium and synovial fluids from normal and patients suffering from orthoarthritis and rheumatoid arthritis. Therefore, therapeutic modulation of this gene may be useful in the treatment of autoimmune and inflammatory diseases such as asthma, allergies, inflammatory bowel disease, lupus erythematosus, psoriasis, rheumatoid arthritis, and osteoarthritis.

General_screening_panel_v1.4 Summary: Ag5057 This gene is expressed at a high to moderate level in pancreatic, gastric, colon cancer and some breast and ovarian cancer cell line with the highest expression seen in a gastric cancer cell line (KATO III, CT=26.33). It Is also expressed at a low level in lung, CNS and prostate cancer cell lines as well as most of the normal tissues on this panel. Hence it may be used as a marker to differentiate cancer cells from normal tissue and therapeutic modulation of the gene product can be used for the treatment of these cancers.

In addition, low levels of expression of this gene is also seen in some regions of central nervous system including fetal brain, cerebellum, thalamus and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Among tissues with metabolic or endocrine function, this gene is expressed at low levels in pancreas, adipose, adrenal gland, thyroid, pituitary gland, liver and the gastrointestinal tract. Therefore, therapeutic modulation of the activity of this gene may prove useful in the treatment of endocrine/metabolically related diseases, such as obesity and diabetes.

Panel 4.1D Summary: Ag5057 Highest expression of this gene is detected in TNF alpha and IL-1 beta treated small airway epithelium (CT=31.7). Expression of this gene is enhanced in cytokine treated small airway epithelium as compared to the resting cells (CT=34). Therefore, modulation of the expression or activity of the protein encoded by this transcript through the application of small molecule therapeutics may be useful in the treatment of asthma, COPD, and emphysema.

Moderate to low levels of expression of this gene is also seen in activated secondary polarized T cells, activated memory T cells, CD8 lymphocytes, resting and IL-2 treated LAK cells, IL-2 treated NK cells, dendritic cells, resting macrophage, activated monocytes, starved HUVEC cells, activated bronchial epithelium, keratinocytes, liver cirrhosis, activated NCI-H292 cells, and normal tissues represented by colon, lung, thymus and kidney. Therefore, therapeutic modulation of this gene may be useful in the treatment of autoimmune and inflammatory diseases such as asthma, allergies, inflammatory bowel disease, lupus erythematosus, psoriasis, rheumatoid arthritis, and osteoarthritis.

W. CG142003-01: Plasma Protease C1 Inhibitor Precursor Protein-Like Protein.

Expression of gene CG142003-01 was assessed using the primer-probe set Ag5686, described in Table WA. Note that CG142003-01 represents a full-length physical clone.

TABLE WA

Probe Name Ag5686

| | | | | |
|---|---|---|---|---|
| | | | Start | |
| Primers | Sequences | Length | Position | SEQ ID No |

| | | | | |
|---|---|---|---|---|
| Forward | 5′-catcgcagaaacctgaagatc- | 21 | 187 | 327 |
| | 3′ | | | |
| Probe | TET-5′- | 26 | 225 | 328 |
| | | taccactgatgaacccaccacacaac-3′-TAMRA | | |
| Reverse | 5′-cagccaccaaaataacagctaa- | 22 | 251 | 329 |
| | 3′ | | | |

AI_comprehensive panel_v1.0 Summary: Ag5686 Expression of this gene is low/undetectable in all samples on this panel (CTs>35).

General_screening_panel_v1.5 Summary: Ag5686 Expression of this gene is low/undetectable in all samples on this panel (CTs>35).

Panel 4.1D Summary: Ag5686 Expression of this gene is low/undetectable in all samples on this panel (CTs>35).

X. CG142023-01: 6230421J19Rik Protein-Like Protein

Expression of gene CG142023-01 was assessed using the primer-probe set Ag7414, described in Table XA.

TABLE XA

Probe Name Ag7414

| | | | | |
|---|---|---|---|---|
| | | | Start | |
| Primers | Sequences | Length | Position | SEQ ID No |

| | | | | |
|---|---|---|---|---|
| Forward | 5′-gaagagcatcgccaccat-3′ | 18 | 798 | 330 |
| Probe | TET-5′-ccctgggctctatcatttactgtgt-3′-TAMRA | 25 | 887 | 331 |
| Reverse | 5′-gctttctggtctccatgaactt- | 22 | 916 | 332 |
| | 3′ | | | |

Y. CG142092-01: C4b-Binding Protein Alpha Chain Precursor Protein-Like Protein.

Expression of gene CG142092-01 was assessed using the primer-probe set Ag6869, described in Table YA. Results of the RTQ-PCR runs are shown in Tables YB and YC. Note that CG142092-01 represents a full-length physical clone.

TABLE YA

Probe Name Ag6869

| | | | | |
|---|---|---|---|---|
| | | | Start | |
| Primers | Sequences | Length | Position | SEQ ID No |

| | | | | |
|---|---|---|---|---|
| Forward | 5′-tcacctacagctgtgaacaa-3′ | 20 | 585 | 333 |
| Probe | TET-5′- | 27 | 612 | 334 |
| | caggcaaaagactcatgcagtgtctcc- | | | |
| | 3′-TAMRA | | | |
| Reverse | 5′-ttttcacatactctgggttt-3′ | 20 | 640 | 335 |

TABLE YB
General_screening_panel_v1.6

| | Rel. Exp. (%) |
|---|---|
| | Ag6869, Run |
| Tissue Name | 278387610 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.3 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 2.8 |
| Ovarian ca. IGROV-1 | 0.0 |
| Ovarian ca. OVCAR-8 | 0.0 |
| Ovary | 0.6 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.3 |
| Trachea | 0.6 |
| Lung | 0.6 |
| Fetal Lung | 2.4 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 1.2 |
| Lung ca. NCI-H526 | 0.0 |
| Lung ca. NCI-H23 | 0.0 |
| Lung ca. NCI-H460 | 0.0 |
| Lung ca. HOP-62 | 1.3 |
| Lung ca. NCI-H522 | 0.0 |
| Liver | 100.0 |
| Fetal Liver | 5.8 |
| Liver ca. HepG2 | 16.2 |
| Kidney Pool | 0.4 |
| Fetal Kidney | 0.2 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 9.8 |
| Bladder | 33.4 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 9.1 |
| Colon cancer tissue | 2.0 |
| Colon ca. SW1116 | 0.0 |
| Colon ca. Colo-205 | 0.9 |
| Colon ca. SW-48 | 0.0 |
| Colon Pool | 0.0 |
| Small Intestine Pool | 0.3 |
| Stomach Pool | 0.0 |
| Bone Marrow Pool | 0.0 |
| Fetal Heart | 0.0 |
| Heart Pool | 0.2 |
| Lymph Node Pool | 0.2 |
| Fetal Skeletal Muscle | 0.5 |
| Skeletal Muscle Pool | 0.5 |
| Spleen Pool | 0.0 |
| Thymus Pool | 0.3 |
| CNS cancel (glio/astro) U87-MG | 0.3 |
| CNS cancer (glio/astro) U-118-MG | 0.0 |
| CNS cancer (neuro, met) SK-N-AS | 0.0 |
| CNS cancer (astro) SF- 539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.7 |
| Brain (fetal) | 0.0 |
| Brain (Hippocampus) Pool | 0.0 |
| Cerebral Cortex Pool | 0.0 |
| Brain (Substantia nigra) Pool | 0.3 |
| Brain (Thalamus) Pool | 0.2 |
| Brain (whole) | 6.2 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 1.1 |

TABLE YC
Panel 4.1D

| | Rel. Exp. (%) |
|---|---|
| | Ag6869, Run |
| Tissue Name | 310594482 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha +IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 1.8 |
| Lung | 23.7 |
| Thymus | 0.0 |
| Kidney | 0.0 |

General_screening_panel_v1.6 Summary: Ag6869 Highest expression of this gene is seen in liver (CT=30). In addition, this gene is expressed at much higher levels in adult liver when compared to expression in the fetal counterpart (CT=34). Thus, expression of this gene may be used to differentiate between the fetal and adult source of this tissue. Low but significant levels of expression are also seen in cancer cell lines derived from liver, renal, and colon cancers, as well as in normal bladder and whole brain. This gene encodes a protein with homology to C4BP, a regulatory protein synthesized by the liver that is involved in the regulation of the classical pathway of complement and the natural anticoagulant pathway. Thus, the restricted pattern of expression of this protein, with highest expression in the liver, is consistent with the its characterization as a novel C4BP.

Panel 4.1D Summary: Ag6869 Low expression of this gene is exclusively seen in liver cirrhosis sample (CT=34). The putative C4b-binding protein encoded for by this gene could potentially allow cells within the liver to respond to specific microenvironmental signals. Therefore, therapeutic modulation of this gene through the use of antibodies or small molecule drug may potentially modulate liver function and play a role in the identification and treatment of inflammatory or autoimmune diseases which effect the liver including liver cirrhosis and fibrosis.

Z. CG142092-02: C4b-Binding Protein Alpha-Chain Precursor Protein-Like Protein.

Expression of gene CG 142092-02 was assessed using the primer-probe set Ag7037, described in Table ZA. Results of the RTQ-PCR runs are shown in Tables ZB and ZC.

TABLE ZA

Probe Name Ag7037

| | | | | |
|---|---|---|---|---|
| Primers | Sequences | Length | Start Position | SEQ ID No |

| | | | | |
|---|---|---|---|---|
| Forward | 5′-gctgttcagaaggctgtgaac-3′ | 21 | 554 | 336 |
| Probe | TET-5′- | 28 | 583 | 337 |
| | | acaggcaaaagactcatgcagtgtctcc | | |
| | -3′-TAMRA | | | |
| Reverse | 5′-ggccattttcacatcctctg-3′ | 20 | 617 | 338 |

TABLE ZB
CNS_neurodegeneration_v1.0

| | Rel. Exp. (%) |
|---|---|
| | Ag7037, Run |
| Tissue Name | 282263012 |
| AD 1 Hippo | 0.0 |
| AD 2 Hippo | 7.5 |
| AD 3 Hippo | 3.7 |
| AD 4 Hippo | 0.0 |
| AD 5 hippo | 63.3 |
| AD 6 Hippo | 46.7 |
| Control 2 Hippo | 0.0 |
| Control 4 Hippo | 2.8 |
| Control (Path) 3 Hippo | 13.0 |
| AD 1 Temporal Ctx | 21.6 |
| AD 2 Temporal Ctx | 11.7 |
| AD 3 Temporal Ctx | 0.0 |
| AD 4 Temporal Ctx | 21.3 |
| AD 5 Inf Temporal Ctx | 59.9 |
| AD 5 Sup Temporal Ctx | 28.9 |
| AD 6 Inf Temporal Ctx | 49.0 |
| AD 6 Sup Temporal Ctx | 45.4 |
| Control 1 Temporal Ctx | 0.0 |
| Control 2 Temporal Ctx | 35.6 |
| Control 3 Temporal Ctx | 24.7 |
| Control 4 Temporal Ctx | 4.3 |
| Control (Path) 1 Temporal Ctx | 6.9 |
| Control (Path) 2 Temporal Ctx | 100.0 |
| Control (Path) 3 Temporal Ctx | 0.0 |
| Control (Path) 4 Temporal Ctx | 37.9 |
| AD 1 Occipital Ctx | 6.8 |
| AD 2 Occipital Ctx (Missing) | 0.0 |
| AD 3 Occipital Ctx | 0.0 |
| AD 4 Occipital Ctx | 0.0 |
| AD 5 Occipital Ctx | 4.1 |
| AD 6 Occipital Ctx | 18.4 |
| Control 1 Occipital Ctx | 4.4 |
| Control 2 Occipital Ctx | 29.9 |
| Control 3 Occipital Ctx | 11.0 |
| Control 4 Occipital Ctx | 16.2 |
| Control (Path) 1 Occipital Ctx | 40.3 |
| Control (Path) 2 Occipital Ctx | 15.2 |
| Control (Path) 3 Occipital Ctx | 0.0 |
| Control (Path) 4 Occipital Ctx | 14.9 |
| Control 1 Parietal Ctx | 16.7 |
| Control 2 Parietal Ctx | 35.8 |
| Control 3 Parietal Ctx | 12.2 |
| Control (Path) 1 Parietal Ctx | 37.6 |
| Control (Path) 2 Parietal Ctx | 4.9 |
| Control (Path) 3 Parietal Ctx | 0.0 |
| Control (Path) 4 Parietal Ctx | 14.4 |

TABLE ZC
Panel 4.1D

| | Rel. Exp. (%) |
|---|---|
| | Ag7037, Run |
| Tissue Name | 282263188 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.3 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK ceils IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.1 |
| EOL-1 dbcAMP | 0.0 |
| EOL-1 dbcAMP PMA/ionomycm | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.1 |
| HUVEC IFN gamma | 0.3 |
| HUVEC TNF alpha + IFN gamma | 0.5 |
| HUVEC TNF alpha + IL4 | 0.1 |
| HUVEC IL-11 | 0.0 |
| Lung Microvascular EC none | 0.3 |
| Lung Microvascular EC TNFalpha + IL-1beta | 0.2 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 100.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.2 |
| HPAEC none | 0.0 |
| HPAEC TNF alpha + IL-1 beta | 0.5 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.2 |
| Lung fibroblast IFN gamma | 0.0 |
| Dermal fibroblast CCD1070 rest | 0.1 |
| Dermal fibroblast CCD1070 TNF alpha | 0.0 |
| Dermal fibroblast CCD1070 IL-1 beta | 0.0 |
| Dermal fibroblast IFN gamma | 0.1 |
| Dermal fibroblast IL-4 | 0.0 |
| Dermal Fibroblasts rest | 0.0 |
| Neutrophils TNFa + LPS | 1.7 |
| Neutrophils rest | 0.2 |
| Colon | 0.0 |
| Lung | 16.4 |
| Thymus | 0.0 |
| Kidney | 0.4 |

CNS_neurodegeneration_v1.0 Summary: Ag7037 This gene is expressed at low levels in the CNS. Therefore, therapeutic modulation of the expression or function of this gene may be useful in the treatment of neurological disorders, such as Alzheimer's disease, Parkinson's disease, schizophrenia, multiple sclerosis, stroke and epilepsy.

Panel 4.1D Summary: Ag7037 Highest expression of this gene is seen in liver cirrhosis (CT=29.6). Thus, expression of this gene could be used to differentiate between this sample and other samples on this panel and as a marker of this condition. Furthermore, therapeutic modulation of the expression or function of this gene may reduce or inhibit fibrosis that occurs in liver cirrhosis.

AA. CG142092-03: C4b-Binding Protein Alpha Chain Precursor Protein-Like Protein.

Expression of gene CG142092-03 was assessed using the primer-probe set Ag7668, described in Table AAA.

TABLE AAA

Probe Name Ag7668

| | | | | |
|---|---|---|---|---|
| Primers | Sequences | Length | Start Position | SEQ ID No |

| | | | | |
|---|---|---|---|---|
| Forward | 5′-tgtggtcctccacccact-3′ | 18 | 286 | 339 |
| Probe | TET-5′- | 29 | 315 | 340 |
| | | tctcagtcaacgtaatatccatcggggc | | |
| | a-3′-TAMRA | | | |
| Reverse | 5′- | 25 | 355 | 341 |
| | gttcaatttccagagtagttccagt-3′ | | | |

CNS_neurodegeneration_v1.0 Summary: Ag7668 Expression of this gene is low/undetectable (CTs>35) across all of the samples on this panel (data not shown).

Panel 4.1D Summary: Ag7668 Expression of this gene is low/undetectable (CTs>35) across all of the samples on this panel (data not shown).

AB. CG51117-03, CG51117-05, CG51117-06 and CG51117-07: Nephronectin-Like Protein

Expression of gene CG51117-03, CG51117-05, and CG51117-06 was assessed using the primer-probe sets Ag2505, Ag2667, Ag2767, Ag2831, Ag5113, Ag5124 and Ag7237, described in Tables ABA, ABB, ABC, ABD, ABE, ABF and ABG. Results of the RTQ-PCR runs are shown in Tables ABH, ABI, ABJ, ABK, ABL, ABM, ABN, ABO, ABP, ABQ, ABR and ABS. Note that Ag5113 is specific for CG51117-07 variant, and Ag5124 is specific for CG51117-06 variant.

TABLE ABA

Probe Name Ag2505

| | | | | |
|---|---|---|---|---|
| | | | Start | |
| Primers | Sequences | Length | Position | SEQ ID No |

| | | | | |
|---|---|---|---|---|
| Forward | 5′-aaagaaggataccagqgtgatg- | 22 | 1113 | 342 |
| | 3′ | | | |
| Probe | TET-5′- | 26 | 1164 | 343 |
| | atgattgaaccttcaggtccaattca-3′-TAMRA | | | |
| Reverse | 5′-ggtaccatttccctttggtaca-3′ | 22 | 1190 | 344 |

TABLE ABB

Probe Name Ag2667

| | | | | |
|---|---|---|---|---|
| | | | Start | |
| Primers | Sequences | Length | Position | SEQ ID No |

| | | | | |
|---|---|---|---|---|
| Forward | 5′-gcagagaatagccaggataagg- | 22 | 434 | 345 |
| | 3′ | | | |
| Probe | TET-5′caaccacgatgcaaacatggtgaat-3′-TAMRA | 25 | 477 | 346 |
| Reverse | 5′-cacttgtttggcccgatac-3′ | 19 | 502 | 347 |

TABLE ABC

Probe Name Ag2767

| | | | | |
|---|---|---|---|---|
| | | | Start | |
| Primers | Sequences | Length | Position | SEQ ID No |

| | | | | |
|---|---|---|---|---|
| Forward | 5′-gcagagaatagccaggataagg- | 22 | 434 | 348 |
| | 3′ | | | |
| Probe | TET-5′-caaccacgatgcaaacatggtgaat-3′-TAMRA | 25 | 477 | 349 |
| Reverse | 5′-cacttgtttggcccgatac-3′ | 19 | 502 | 350 |

TABLE ABD

Probe Name Ag2831

| | | | | |
|---|---|---|---|---|
| | | | Start | |
| Primers | Sequences | Length | Position | SEQ ID No |

| | | | | |
|---|---|---|---|---|
| Forward | 5′-gcagagaatagccaggataagg- | 22 | 434 | 351 |
| | 3′ | | | |
| Probe | TET-5′-caaccacgatgcaaacatggtgaat-3′-TAMRA | 25 | 477 | 352 |
| Reverse | 5′-cacttgtttggcccgatac-3′ | 19 | 502 | 353 |

TABLE ABE

Probe Name Ag5113

| | | | | |
|---|---|---|---|---|
| | | | Start | |
| Primers | Sequences | Length | Position | SEQ ID No |

| | | | | |
|---|---|---|---|---|
| Forward | 5′-gtcagcctgtgtgccaa-3′ | 17 | 412 | 354 |
| Probe | TET-5′- | 26 | 459 | 355 |
| | ccaaacaagtgcaagtgtcatcctgg-3′-TAMRA | | | |
| Reverse | 5′-gggatgtgctcgtcttga-3′ | 18 | 506 | 356 |

TABLE ABF

Probe Name Ag5124

| | | | | |
|---|---|---|---|---|
| | | | Start | |
| Primers | Sequences | Length | Position | SEQ ID No |

| | | | | |
|---|---|---|---|---|
| Forward | 5′-aggataaggtgccagctca-3′ | 19 | 447 | 357 |
| Probe | TET-5′- | 26 | 510 | 358 |
| | ccaaacaagtgcaagtgtcatcctgg-3′-TAMRA | | | |
| Reverse | 5′-gggatgtgctcgtcttga-3′ | 18 | 557 | 359 |

TABLE ABG

Probe Name Ag7237

| | | | | |
|---|---|---|---|---|
| | | | Start | |
| Primers | Sequences | Length | Position | SEQ ID No |

| | | | | |
|---|---|---|---|---|
| Forward | 5′-gtgttcattccacggcaac-3′ | 19 | 1539 | 360 |
| Probe | TET-5′- | 27 | 1588 | 361 |
| | catcgtctgcactgactcctctttcta-3′-TAMRA | | | |
| Reverse | 5′-gtgtaccagaacacctggatca-3′ | 22 | 1625 | 362 |

TABLE ABH
AI_comprehensive panel_v1.0

| | Rel. Exp. (%) | Rel. Exp. (%) |
|---|---|---|
| | Ag2505, Run | Ag2831, Run |
| Tissue Name | 248588456 | 244570250 |
| 110967 COPD-F | 15.3 | 9.3 |
| 110980 COPD-F | 11.8 | 7.0 |
| 110968 COPD-M | 8.9 | 5.8 |
| 110977 COPD-M | 28.1 | 14.1 |
| 110989 Emphysema-F | 9.6 | 12.2 |
| 110992 Emphysema-F | 1.9 | 4.1 |
| 110993 Emphysema-F | 7.7 | 9.3 |
| 110994 Emphysema-F | 5.2 | 4.1 |
| 110995 Emphysema-F | 3.6 | 4.3 |
| 110996 Emphysema-F | 0.4 | 0.2 |
| 110997 Asthma-M | 4.6 | 3.0 |
| 111001 Asthma-F | 2.3 | 5.0 |
| 111002 Asthma-F | 3.5 | 7.2 |
| 111003 Atopic Asthma-F | 22.5 | 22.2 |
| 111004 Atopic Asthma-F | 10.4 | 11.4 |
| 111005 Atopic Asthma-F | 7.3 | 9.4 |
| 111006 Atopic Asthma-F | 1.6 | 1.4 |
| 111417 Allergy-M | 5.1 | 2.8 |
| 112347 Allergy-M | 1.4 | 0.2 |
| 112349 Normal Lung-F | 0.7 | 0.2 |
| 112357 Normal Lung-F | 7.1 | 6.4 |
| 112354 Normal Lung-M | 7.6 | 6.0 |
| 112374 Crohns-F | 9.0 | 3.2 |
| 112389 Match | 11.2 | 6.6 |
| Control Crohns-F | | |
| 112375 Crohns-F | 10.2 | 6.0 |
| 112732 Match | 1.2 | 2.1 |
| Control Crohns-F | | |
| 112725 Crohns-M | 0.9 | 1.6 |
| 112387 Match | 11.4 | 13.0 |
| Control Crohns-M | | |
| 112378 Crohns-M | 1.3 | 2.0 |
| 112390 Match | 16.7 | 4.3 |
| Control Crohns-M | | |
| 112726 Crohns-M | 21.8 | 17.0 |
| 112731 Match | 15.3 | 6.3 |
| Control Crohns-M | | |
| 112380 Ulcer Col-F | 5.8 | 7.0 |
| 112734 Match | 3.7 | 5.0 |
| Control Ulcer Col-F | | |
| 112384 Ulcer Col-F | 19.2 | 15.0 |
| 112737 Match | 13.5 | 12.0 |
| Control Ulcer Col-F | | |
| 112386 Ulcer Col-F | 8.4 | 6.0 |
| 112738 Match | 3.8 | 2.1 |
| Control Ulcer Col-F | | |
| 112381 Ulcer Col-M | 5.0 | 9.9 |
| 112735 Match Control | 9.7 | 5.8 |
| Ulcer Col-M | | |
| 112382 Ulcer Col-M | 11.7 | 12.6 |
| 112394 Match Control | 3.1 | 3.4 |
| Ulcer Col-M | | |
| 112383 Ulcer Col-M | 5.1 | 13.7 |
| 112736 Match Control | 5.0 | 6.3 |
| Ulcer Col-M | | |
| 112423 Psoriasis-F | 14.0 | 10.3 |
| 112427 Match | 16.7 | 18.9 |
| Control Psoriasis-F | | |
| 112418 Psoriasis-M | 14.0 | 13.9 |
| 112723 Match | 0.2 | 0.2 |
| Control Psoriasis-M | | |
| 112419 Psoriasis-M | 18.2 | 8.7 |
| 112424 Match | 6.8 | 6.7 |
| Control Psoriasis-M | | |
| 112420 Psoriasis-M | 13.9 | 15.5 |
| 112425 Match | 13.6 | 16.6 |
| Control Psoriasis-M | | |
| 104689 (MF) OA | 25.3 | 38.4 |
| Bone-Backus | | |
| 104690 (MF) Adj | 27.9 | 21.2 |
| “Normal” Bone- | | |
| Backus | | |
| 104691 (MF) OA | 2.9 | 3.0 |
| Synovium-Backus | | |
| 104692 (BA) OA | 0.0 | 0.0 |
| Cartilage-Backus | | |
| 104694 (BA) OA | 5.8 | 18.7 |
| Bone-Backus | | |
| 104695 (BA) Adj | 14.1 | 19.8 |
| “Normal” Bone-Backus | | |
| 104696 (BA) OA | 2.3 | 3.6 |
| Synovium-Backus | | |
| 104700 (SS) OA | 28.9 | 22.4 |
| Bone-Backus | | |
| 104701 (SS) Adj | 25.5 | 18.7 |
| “Normal” Bone-Backus | | |
| 104702 (SS) OA | 11.7 | 7.1 |
| Synovium-Backus | | |
| 117093 OA | 7.5 | 7.5 |
| Cartilage Rep7 | | |
| 112672 OA Bone5 | 19.2 | 17.2 |
| 112673 OA Synovium5 | 6.4 | 3.8 |
| 112674 OA | 6.8 | 4.2 |
| Synovial Fluid cells5 | | |
| 117100 OA | 1.9 | 2.1 |
| Cartilage Repl4 | | |
| 112756 OA Bone9 | 26.4 | 31.6 |
| 112757 OA Synovium9 | 2.8 | 1.3 |
| 112758 OA | 8.1 | 6.3 |
| Synovial Fluid | | |
| Cells9 | | |
| 117125 RA | 14.8 | 9.2 |
| Cartilage Rep2 | | |
| 113492 Bone2 RA | 84.7 | 47.0 |
| 113493 Synovium2 RA | 40.9 | 25.3 |
| 113494 Syn Fluid | 61.1 | 49.3 |
| Cells RA | | |
| 113499 Cartilage4 RA | 90.1 | 73.2 |
| 113500 Bone4 RA | 100.0 | 100.0 |
| 113501 Synovium4 RA | 71.2 | 59.5 |
| 113502 Syn Fluid | 48.6 | 37.9 |
| Cells4 RA | | |
| 113495 Cartilage3 RA | 77.9 | 47.0 |
| 113496 Bone3 RA | 92.0 | 41.8 |
| 113497 Synovium3 RA | 53.6 | 24.0 |
| 113498 Syn Fluid | 98.6 | 57.0 |
| Cells3 RA | | |
| 117106 Normal | 3.0 | 1.6 |
| Cartilage Rep20 | | |
| 113663 Bone3 Normal | 2.0 | 0.7 |
| 113664 Synovium3 | 0.5 | 0.4 |
| Normal | | |
| 113665 Syn Fluid | 1.6 | 1.4 |
| Cells3 Normal | | |
| 117107 Normal | 3.7 | 5.5 |
| Cartilage Rep22 | | |
| 113667 Bone4 Normal | 4.3 | 6.0 |
| 113668 Synovium4 | 9.1 | 7.4 |
| Normal | | |
| 113669 Syn Fluid | 6.6 | 6.6 |
| Cells4 Normal | | |

TABLE ABI
CNS_neurodegeneration_v1.0

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag2505, | Ag2505, | Ag2667, | Ag2767, | Ag2831, | Ag7237, |
| | Run | Run | Run | Run | Run | Run |
| Tissue Name | 208123723 | 224116291 | 206955569 | 206985756 | 208699692 | 296423778 |
| AD 1 Hippo | 14.1 | 19.1 | 42.9 | 27.4 | 29.1 | 11.7 |
| AD 2 Hippo | 29.3 | 40.3 | 58.2 | 37.1 | 56.3 | 36.6 |
| AD 3 Hippo | 5.1 | 8.5 | 9.0 | 5.6 | 2.9 | 7.8 |
| AD 4 Hippo | 10.4 | 10.1 | 13.4 | 21.2 | 8.8 | 11.4 |
| AD 5 Hippo | 43.8 | 47.6 | 52.1 | 35.4 | 40.3 | 43.2 |
| AD 6 Hippo | 100.0 | 100.0 | 98.6 | 100.0 | 79.0 | 100.0 |
| Control 2 | 15.3 | 19.6 | 5.1 | 19.6 | 5.5 | 11.0 |
| Hippo | | | | | | |
| Control 4 | 15.6 | 21.0 | 16.0 | 15.2 | 25.7 | 32.8 |
| Hippo | | | | | | |
| Control (Path) | 4.8 | 5.8 | 22.1 | 2.7 | 4.5 | 6.5 |
| 3 Hippo | | | | | | |
| AD 1 | 21.5 | 26.4 | 40.9 | 15.6 | 24.5 | 22.2 |
| Temporal Ctx | | | | | | |
| AD 2 | 28.5 | 27.9 | 52.5 | 27.0 | 84.7 | 35.8 |
| Temporal Ctx | | | | | | |
| AD 3 | 9.3 | 8.5 | 13.4 | 7.3 | 3.2 | 2.1 |
| Temporal Ctx | | | | | | |
| AD 4 | 26.1 | 35.1 | 59.0 | 18.2 | 30.8 | 29.1 |
| Temporal Ctx | | | | | | |
| AD 5 Inf | 28.9 | 33.9 | 39.5 | 27.0 | 49.3 | 37.1 |
| Temporal Ctx | | | | | | |
| AD 5 Sup | 38.4 | 40.6 | 23.0 | 17.2 | 54.7 | 45.7 |
| Temporal Ctx | | | | | | |
| AD 6 Inf | 83.5 | 96.6 | 100.0 | 66.4 | 100.0 | 94.0 |
| Temporal Ctx | | | | | | |
| AD 6 Sup | 70.7 | 90.8 | 99.3 | 43.5 | 62.4 | 82.9 |
| Temporal Ctx | | | | | | |
| Control 1 | 4.2 | 4.2 | 17.3 | 7.7 | 3.1 | 1.9 |
| Temporal Ctx | | | | | | |
| Control 2 | 10.6 | 14.0 | 12.1 | 25.5 | 18.6 | 15.5 |
| Temporal Ctx | | | | | | |
| Control 3 | 3.1 | 5.6 | 0.0 | 0.0 | 3.6 | 10.7 |
| Temporal Ctx | | | | | | |
| Control 3 | 6.5 | 14.6 | 12.5 | 18.2 | 19.2 | 16.6 |
| Temporal Ctx | | | | | | |
| Control (Path) | 18.0 | 21.6 | 43.2 | 26.6 | 19.3 | 21.9 |
| 1 Temporal | | | | | | |
| Ctx | | | | | | |
| Control (Path) | 13.9 | 22.1 | 26.1 | 42.3 | 42.0 | 21.5 |
| 2 Temporal | | | | | | |
| Ctx | | | | | | |
| Control (Path) | 3.2 | 4.2 | 4.4 | 7.0 | 11.8 | 4.6 |
| 3 Temporal | | | | | | |
| Ctx | | | | | | |
| Control (Path) | 13.4 | 15.3 | 26.6 | 21.9 | 13.8 | 19.6 |
| 4 Temporal | | | | | | |
| Ctx | | | | | | |
| AD 1 Occipital | 13.4 | 15.2 | 27.9 | 7.9 | 16.2 | 13.5 |
| Ctx | | | | | | |
| AD 2 Occipital | 0.0 | 0.0 | 0.0 | 0.0 | 0.0 | 0.0 |
| Ctx (Missing) | | | | | | |
| AD 3 Occipital | 4.1 | 5.8 | 11.8 | 0.0 | 9.9 | 3.0 |
| Ctx | | | | | | |
| AD 4 Occipital | 19.3 | 23.2 | 17.0 | 9.0 | 41.2 | 27.4 |
| Ctx | | | | | | |
| AD 5 Occipital | 17.8 | 16.8 | 39.5 | 27.9 | 17.6 | 19.6 |
| Ctx | | | | | | |
| AD 6 Occipital | 29.3 | 43.8 | 30.8 | 25.5 | 13.2 | 32.8 |
| Ctx | | | | | | |
| Control 1 | 4.0 | 3.0 | 14.0 | 0.0 | 13.0 | 2.8 |
| Occipital Ctx | | | | | | |
| Control 2 | 21.8 | 25.3 | 2.3 | 29.1 | 17.7 | 32.3 |
| Occipital Ctx | | | | | | |
| Control 3 | 6.9 | 7.3 | 28.7 | 4.8 | 7.1 | 9.2 |
| Occipital Ctx | | | | | | |
| Control 4 | 9.4 | 10.3 | 13.8 | 9.1 | 17.6 | 17.7 |
| Occipital Ctx | | | | | | |
| Control (Path) | 29.1 | 28.1 | 37.6 | 29.3 | 47.6 | 34.2 |
| 1 Occipital Ctx | | | | | | |
| Control (Path) | 5.1 | 7.0 | 6.7 | 5.3 | 8.6 | 5.3 |
| 2 Occipital Ctx | | | | | | |
| Control (Path) | 1.6 | 2.5 | 25.7 | 3.5 | 0.0 | 2.4 |
| 3 Occipital Ctx | | | | | | |
| Control (Path) | 13.7 | 17.2 | 19.8 | 13.5 | 13.7 | 12.2 |
| 4 Occipital Ctx | | | | | | |
| Control 1 | 3.8 | 4.0 | 10.8 | 24.1 | 7.3 | 3.8 |
| Parietal Ctx | | | | | | |
| Control 2 | 37.4 | 47.6 | 53.6 | 36.1 | 57.4 | 53.6 |
| Parietal Ctx | | | | | | |
| Control 3 | 4.1 | 5.4 | 0.0 | 3.4 | 3.5 | 5.3 |
| Parietal Ctx | | | | | | |
| Control (Path) | 23.5 | 28.9 | 24.7 | 21.5 | 42.6 | 30.4 |
| 1 Parietal Ctx | | | | | | |
| Control (Path) | 15.7 | 20.2 | 44.8 | 11.5 | 39.5 | 20.6 |
| 2 Parietal Ctx | | | | | | |
| Control (Path) | 2.6 | 4.0 | 14.9 | 3.7 | 3.0 | 2.4 |
| 3 Parietal Ctx | | | | | | |
| Control (Path) | 21.9 | 25.7 | 49.7 | 20.4 | 50.7 | 26.2 |
| 4 Parietal Ctx | | | | | | |

TABLE ABJ
General_screening_panel_v1.5

| | Rel. Exp. (%) | Rel. Exp. (%) |
|---|---|---|
| | Ag5113, Run | Ag5124, Run |
| Tissue Name | 228738816 | 228745551 |
| Adipose | 3.6 | 3.3 |
| Melanoma* Hs688(A).T | 0.0 | 0.0 |
| Melanoma* Hs688(B).T | 0.0 | 0.0 |
| Melanoma* M14 | 0.0 | 0.0 |
| Melanoma* LOXIMVI | 0.0 | 0.0 |
| Melanoma* SK-MEL-5 | 0.0 | 0.0 |
| Squamous cell | 0.0 | 0.0 |
| carcinoma SCC-4 | | |
| Testis Pool | 3.9 | 5.9 |
| Prostate ca.* (bone | 0.1 | 0.0 |
| met) PC-3 | | |
| Prostate Pool | 7.3 | 5.0 |
| Placenta | 0.1 | 0.0 |
| Uterus Pool | 4.7 | 4.1 |
| Ovarian ca. OVCAR-3 | 0.0 | 0.0 |
| Ovarian ca. SK-OV-3 | 0.2 | 0.0 |
| Ovarian ca. OVCAR-4 | 0.1 | 0.0 |
| Ovarian ca. OVCAR-5 | 0.0 | 0.0 |
| Ovarian ca. IGROV-1 | 0.0 | 1.4 |
| Ovarian ca. OVCAR-8 | 0.3 | 0.0 |
| Ovary | 0.5 | 2.5 |
| Breast ca. MCF-7 | 0.2 | 0.6 |
| Breast ca. MDA-MB-231 | 0.0 | 0.0 |
| Breast ca. BT 549 | 0.3 | 1.4 |
| Breast ca. T47D | 0.0 | 0.0 |
| Breast ca. MDA-N | 0.0 | 0.0 |
| Breast Pool | 4.2 | 3.7 |
| Trachea | 6.0 | 4.2 |
| Lung | 3.2 | 2.3 |
| Fetal Lung | 100.0 | 100.0 |
| Lung ca. NCI-N417 | 0.0 | 0.0 |
| Lung ca. LX-1 | 0.0 | 0.0 |
| Lung ca. NCI-H146 | 0.0 | 0.0 |
| Lung ca. SHP-77 | 0.0 | 0.0 |
| Lung ca. A549 | 0.0 | 0.0 |
| Lung ca. NCI-H526 | 0.0 | 0.0 |
| Lung ca. NCI-H23 | 0.0 | 0.0 |
| Lung ca. NCI-H460 | 0.1 | 0.0 |
| Lung ca. HOP-62 | 0.0 | 0.0 |
| Lung ca. NCI-H522 | 0.0 | 0.0 |
| Liver | 0.0 | 0.0 |
| Fetal Liver | 0.2 | 0.0 |
| Liver ca. HepG2 | 0.0 | 0.0 |
| Kidney Pool | 13.2 | 8.2 |
| Fetal Kidney | 0.3 | 1.5 |
| Renal ca. 786-0 | 0.0 | 0.0 |
| Renal ca. A498 | 0.0 | 0.0 |
| Renal ca. ACHN | 1.3 | 0.4 |
| Renal ca. UO-31 | 0.0 | 0.0 |
| Renal ca. TK-10 | 0.0 | 0.0 |
| Bladder | 1.2 | 2.4 |
| Gastric ca. (liver | 0.1 | 0.0 |
| met.) NCI-N87 | | |
| Gastric ca. KATO III | 0.1 | 0.0 |
| Colon ca. SW-948 | 0.0 | 0.0 |
| Colon ca. SW480 | 0.0 | 0.0 |
| Colon ca.* (SW480 met) | 0.0 | 0.0 |
| SW620 | | |
| Colon ca. HT29 | 0.0 | 0.0 |
| Colon ca. HCT-116 | 0.1 | 0.5 |
| Colon ca. CaCo-2 | 0.0 | 0.0 |
| Colon cancer tissue | 0.5 | 1.3 |
| Colon ca. SW1116 | 0.0 | 0.4 |
| Colon ca. Colo-205 | 0.1 | 0.0 |
| Colon ca. SW-48 | 0.0 | 0.0 |
| Colon Pool | 2.1 | 5.5 |
| Small Intestine Pool | 2.4 | 2.8 |
| Stomach Pool | 2.0 | 3.2 |
| Bone Marrow Pool | 3.0 | 1.8 |
| Fetal Heart | 0.2 | 0.0 |
| Heart Pool | 2.4 | 1.9 |
| Lymph Node Pool | 8.5 | 9.3 |
| Fetal Skeletal Muscle | 1.4 | 2.1 |
| Skeletal Muscle Pool | 1.1 | 1.7 |
| Spleen Pool | 0.3 | 0.0 |
| Thymus Pool | 1.1 | 0.0 |
| CNS cancer | 0.0 | 0.0 |
| (glio/astro) U87-MG | | |
| CNS cancer | 0.0 | 0.9 |
| (glio/astro) U-118-MG | | |
| CNS cancer | 0.0 | 0.0 |
| (neuro; met) SK-N-AS | | |
| CNS cancer (astro) SF-539 | 0.3 | 0.5 |
| CNS cancer (astro) SNB-75 | 0.0 | 0.6 |
| CNS cancer (glio) SNB-19 | 0.2 | 1.0 |
| CNS cancer (glio) SF-295 | 0.0 | 1.9 |
| Brain (Amygdala) Pool | 0.0 | 0.0 |
| Brain (cerebellum) | 0.0 | 0.0 |
| Brain (fetal) | 0.2 | 0.5 |
| Brain (Hippocampus) Pool | 0.5 | 0.8 |
| Cerebral Cortex Pool | 0.3 | 0.0 |
| Brain (Substantia nigra) Pool | 0.1 | 0.0 |
| Brain (Thalamus) Pool | 0.0 | 0.0 |
| Brain (whole) | 0.3 | 0.5 |
| Spinal Cord Pool | 0.0 | 0.4 |
| Adrenal Gland | 0.6 | 2.0 |
| Pituitary gland Pool | 0.2 | 0.0 |
| Salivary Gland | 0.0 | 0.0 |
| Thyroid (female) | 0.3 | 0.4 |
| Pancreatic ca. CAPAN2 | 0.0 | 0.0 |
| Pancreas Pool | 1.7 | 3.8 |

TABLE ABK
General_screening_panel_v1.6

| | Rel. Exp. (%) |
|---|---|
| | Ag7237, Run |
| Tissue Name | 296433071 |
| Adipose | 19.9 |
| Melanoma* Hs688(A).T | 0.2 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 1.9 |
| Testis Pool | 5.0 |
| Prostate ca.* (bone met) PC-3 | 0.3 |
| Prostate Pool | 44.1 |
| Placenta | 1.0 |
| Uterus Pool | 2.0 |
| Ovarian ca. OVCAR-3 | 5.6 |
| Ovarian ca. SK-OV-3 | 18.0 |
| Ovarian ca. OVCAR-4 | 0.0 |
| Ovarian ca. OVCAR-5 | 4.7 |
| Ovarian ca. IGROV-1 | 10.9 |
| Ovarian ca. OVCAR-8 | 0.9 |
| Ovary | 2.3 |
| Breast ca. MCF-7 | 71.7 |
| Breast ca. MDA-MB-231 | 0.0 |
| Breast ca. BT 549 | 12.2 |
| Breast ca. T47D | 6.2 |
| Breast ca. MDA-N | 0.0 |
| Breast Pool | 7.5 |
| Trachea | 10.7 |
| Lung | 2.4 |
| Fetal Lung | 100.0 |
| Lung ca. NCI-N417 | 0.0 |
| Lung ca. LX-1 | 5.2 |
| Lung ca. NCI-H146 | 7.9 |
| Lung ca. SHP-77 | 0.7 |
| Lung ca. A549 | 0.7 |
| Lung ca. NCI-H526 | 0.3 |
| Lung ca. NCI-H23 | 4.5 |
| Lung ca. NCI-H460 | 0.5 |
| Lung ca. HOP-62 | 0.0 |
| Lung ca. NCI-H522 | 0.0 |
| Liver | 0.0 |
| Fetal Liver | 1.3 |
| Liver ca. HepG2 | 2.7 |
| Kidney Pool | 0.0 |
| Fetal Kidney | 23.2 |
| Renal ca. 786-0 | 0.0 |
| Renal ca. A498 | 0.0 |
| Renal ca. ACHN | 47.6 |
| Renal ca. UO-31 | 0.0 |
| Renal ca. TK-10 | 2.4 |
| Bladder | 19.2 |
| Gastric ca. (liver met.) NCI-N87 | 45.7 |
| Gastric ca. KATO III | 11.7 |
| Colon ca. SW-948 | 9.1 |
| Colon ca. SW480 | 0.5 |
| Colon ca.* (SW480 met) SW620 | 0.0 |
| Colon ca. HT29 | 12.4 |
| Colon ca. HCT-116 | 10.0 |
| Colon ca. CaCo-2 | 17.4 |
| Colon cancer tissue | 5.9 |
| Colon ca. SW1116 | 4.8 |
| Colon ca. Colo-205 | 4.2 |
| Colon ca. SW-48 | 10.1 |
| Colon Pool | 5.8 |
| Small Intestine Pool | 6.3 |
| Stomach Pool | 4.3 |
| Bone Marrow Pool | 6.4 |
| Fetal Heart | 6.0 |
| Heart Pool | 4.5 |
| Lymph Node Pool | 18.0 |
| Fetal Skeletal Muscle | 12.8 |
| Skeletal Muscle Pool | 0.6 |
| Spleen Pool | 5.7 |
| Thymus Pool | 6.9 |
| CNS cancer (glio/astro) U87-MG | 0.0 |
| CNS cancer (glio/astro) U-118-MG | 0.0 |
| CNS cancer (neuro; met) SK-N-AS | 0.1 |
| CNS cancer (astro) SF-539 | 1.7 |
| CNS cancer (astro) SNB-75 | 0.7 |
| CNS cancer (glio) SNB-19 | 12.7 |
| CNS cancer (glio) SF-295 | 0.2 |
| Brain (Amygdala) Pool | 3.0 |
| Brain (cerebellum) | 0.6 |
| Brain (fetal) | 24.8 |
| Brain (Hippocampus) Pool | 7.5 |
| Cerebral Cortex Pool | 3.7 |
| Brain (Substantia nigra) Pool | 1.9 |
| Brain (Thalamus) Pool | 5.5 |
| Brain (whole) | 6.1 |
| Spinal Cord Pool | 1.0 |
| Adrenal Gland | 3.1 |
| Pituitary gland Pool | 5.8 |
| Salivary Gland | 0.7 |
| Thyroid (female) | 47.3 |
| Pancreatic ca. CAPAN 2 | 0.7 |
| Pancreas Pool | 9.6 |

TABLE ABL
Panel 1.3D

| | Rel. Exp. (%) | Rel. Exp. (%) | Rel. Exp. (%) | Rel. Exp. (%) |
|---|---|---|---|---|
| | Ag2505, Run | Ag2667, Run | Ag2767, Run | Ag2831, Run |
| Tissue Name | 165531061 | 162554578 | 165527179 | 165517578 |
| Liver adenocarcinoma | 1.8 | 0.0 | 0.0 | 1.3 |
| Pancreas | 13.7 | 8.9 | 35.4 | 14.1 |
| Pancreatic ca. CAPAN2 | 1.5 | 2.0 | 0.0 | 2.0 |
| Adrenal gland | 3.6 | 2.9 | 2.8 | 4.6 |
| Thyroid | 100.0 | 52.5 | 100.0 | 67.8 |
| Salivary gland | 4.1 | 2.3 | 9.3 | 2.5 |
| Pituitary gland | 37.6 | 9.9 | 18.7 | 19.8 |
| Brain (fetal) | 44.1 | 6.8 | 40.9 | 28.5 |
| Brain (whole) | 9.3 | 0.6 | 11.2 | 0.0 |
| Brain (amygdala) | 8.1 | 4.4 | 8.2 | 2.6 |
| Brain (cerebellum) | 1.8 | 0.8 | 0.0 | 4.4 |
| Brain (hippocampus) | 10.2 | 1.6 | 6.4 | 2.2 |
| Brain (Substantia nigra) | 29.3 | 3.7 | 12.5 | 11.0 |
| Brain (thalamus) | 3.6 | 1.9 | 8.7 | 7.2 |
| Cerebral Cortex | 7.7 | 8.8 | 3.8 | 1.3 |
| Spinal cord | 15.2 | 14.4 | 13.3 | 10.4 |
| glio/astro U87-MG | 0.0 | 0.0 | 0.0 | 0.0 |
| glio/astro U-118-MG | 0.0 | 0.0 | 0.0 | 0.0 |
| astrocytoma SW1783 | 0.3 | 0.6 | 0.0 | 1.0 |
| neuro*; met SK-N-AS | 0.4 | 0.0 | 0.0 | 0.0 |
| astrocytoma SF-539 | 1.8 | 1.2 | 2.5 | 1.2 |
| astrocytoma SNB-75 | 2.7 | 0.6 | 0.0 | 2.0 |
| glioma SNB-19 | 0.0 | 0.0 | 0.0 | 0.0 |
| glioma U251 | 9.3 | 2.0 | 12.2 | 9.1 |
| glioma SF-295 | 0.4 | 0.0 | 2.6 | 1.3 |
| Heart (fetal) | 10.0 | 24.7 | 9.6 | 7.4 |
| Heart | 3.1 | 0.0 | 2.4 | 2.4 |
| Skeletal muscle (fetal) | 12.8 | 66.4 | 7.7 | 1.3 |
| Skeletal muscle | 20.9 | 2.1 | 7.5 | 13.1 |
| Bone marrow | 1.2 | 1.9 | 4.5 | 0.9 |
| Thymus | 6.0 | 24.0 | 17.8 | 4.9 |
| Spleen | 6.7 | 5.0 | 19.8 | 9.2 |
| Lymph node | 6.7 | 1.4 | 5.4 | 2.6 |
| Colorectal | 23.5 | 19.3 | 6.5 | 9.9 |
| Stomach | 12.0 | 1.8 | 4.5 | 2.5 |
| Small intestine | 54.3 | 13.3 | 69.7 | 43.2 |
| Colon ca. SW480 | 1.1 | 0.5 | 0.0 | 0.0 |
| Colon ca.* | 1.4 | 0.0 | 2.8 | 3.1 |
| SW620(SW480 met) | | | | |
| Colon ca. HT29 | 7.3 | 28.3 | 11.3 | 5.3 |
| Colon ca. HCT-116 | 7.3 | 9.0 | 12.4 | 10.2 |
| Colon ca. CaCo-2 | 10.7 | 29.7 | 19.6 | 11.1 |
| Colon ca. | 8.5 | 14.6 | 10.6 | 10.6 |
| tissue(ODO3866) | | | | |
| Colon ca. HCC-2998 | 2.9 | 6.3 | 19.8 | 2.9 |
| Gastric ca.* (liver met) | 71.7 | 49.7 | 95.3 | 100.0 |
| NCI-N87 | | | | |
| Bladder | 14.8 | 44.4 | 29.7 | 20.4 |
| Trachea | 21.9 | 13.9 | 18.6 | 5.5 |
| Kidney | 38.2 | 74.2 | 56.3 | 67.4 |
| Kidney (fetal) | 27.5 | 37.1 | 40.1 | 33.9 |
| Renal ca. 786-0 | 0.0 | 0.0 | 0.0 | 0.0 |
| Renal ca. A498 | 0.2 | 1.7 | 3.7 | 2.9 |
| Renal ca. RXF 393 | 39.8 | 5.9 | 20.3 | 10.8 |
| Renal ca. ACHN | 51.1 | 6.8 | 20.2 | 7.2 |
| Renal ca. UO-31 | 0.2 | 0.0 | 0.0 | 0.0 |
| Renal ca. TK-10 | 0.0 | 0.0 | 0.0 | 0.0 |
| Liver | 1.4 | 0.7 | 0.0 | 3.5 |
| Liver (fetal) | 2.3 | 0.0 | 4.0 | 1.2 |
| Liver ca. (hepatoblast) | 11.8 | 4.7 | 19.5 | 10.8 |
| HepG2 | | | | |
| Lung | 75.3 | 46.0 | 91.4 | 84.1 |
| Lung (fetal) | 54.7 | 100.0 | 92.0 | 64.6 |
| Lung ca. (small cell) | 5.5 | 2.5 | 5.8 | 4.1 |
| LX-1 | | | | |
| Lung ca. (small cell) | 5.6 | 6.2 | 10.1 | 5.3 |
| NCI-H69 | | | | |
| Lung ca. (s. cell var.) | 0.2 | 0.6 | 0.0 | 0.0 |
| SHP-77 | | | | |
| Lung ca. (large | 1.2 | 0.0 | 0.0 | 0.0 |
| cell)NCI-H460 | | | | |
| Lung ca. (non-sm. cell) | 1.2 | 0.7 | 3.1 | 1.3 |
| A549 | | | | |
| Lung ca. (non-s. cell) | 3.2 | 4.7 | 8.2 | 4.5 |
| NCI-H23 | | | | |
| Lung ca. (non-s. cell) | 0.0 | 0.0 | 0.0 | 0.0 |
| HOP-62 | | | | |
| Lung ca. (non-s. cl) | 0.0 | 0.0 | 0.0 | 0.0 |
| NCI-H522 | | | | |
| Lung ca. (squam.) SW | 1.8 | 0.0 | 3.4 | 2.0 |
| 900 | | | | |
| Lung ca. (squam.) NCI- | 14.6 | 10.9 | 31.6 | 53.6 |
| H596 | | | | |
| Mammary gland | 11.9 | 4.9 | 23.8 | 17.4 |
| Breast ca.* (pl. ef) | 89.5 | 92.7 | 84.1 | 80.1 |
| MCF-7 | | | | |
| Breast ca.* (pl. ef) | 0.0 | 0.0 | 0.0 | 0.0 |
| MDA-MB-231 | | | | |
| Breast ca.* (pl. ef) | 24.7 | 7.6 | 20.3 | 9.9 |
| T47D | | | | |
| Breast ca. BT-549 | 2.3 | 0.0 | 0.0 | 1.2 |
| Breast ca. MDA-N | 0.0 | 0.0 | 0.0 | 0.0 |
| Ovary | 3.5 | 20.3 | 8.5 | 4.9 |
| Ovarian ca. OVCAR-3 | 6.4 | 2.6 | 8.2 | 4.5 |
| Ovarian ca. OVCAR-4 | 0.0 | 0.0 | 0.0 | 0.0 |
| Ovarian ca. OVCAR-5 | 0.0 | 0.0 | 0.0 | 0.0 |
| Ovarian ca. OVCAR-8 | 0.2 | 1.0 | 0.0 | 0.0 |
| Ovarian ca. IGROV-1 | 14.5 | 17.6 | 31.6 | 33.7 |
| Ovarian ca.* (ascites) | 9.3 | 5.4 | 20.7 | 22.5 |
| SK-OV-3 | | | | |
| Uterus | 27.7 | 3.5 | 39.0 | 46.3 |
| Placenta | 2.9 | 4.9 | 6.4 | 8.9 |
| Prostate | 25.0 | 8.8 | 16.7 | 16.7 |
| Prostate ca.* (bone | 0.0 | 0.0 | 0.0 | 0.0 |
| met)PC-3 | | | | |
| Testis | 2.5 | 0.7 | 4.4 | 2.7 |
| Melanoma Hs688(A).T | 0.0 | 0.0 | 0.0 | 0.0 |
| Melanoma* (met) | 0.0 | 0.0 | 0.0 | 0.0 |
| Hs688(B).T | | | | |
| Melanoma UACC-62 | 0.0 | 0.0 | 0.0 | 0.0 |
| Melanoma M14 | 0.0 | 0.0 | 0.0 | 0.0 |
| Melanoma LOX IMVI | 0.0 | 0.0 | 0.0 | 0.0 |
| Melanoma* (met) SK- | 0.0 | 0.0 | 0.0 | 0.0 |
| MEL-5 | | | | |
| Adipose | 19.3 | 6.5 | 4.3 | 22.1 |

TABLE ABM
Panel 2.2

| | Rel. Exp. (%) |
|---|---|
| | Ag2831, Run |
| Tissue Name | 175063921 |
| Normal Colon | 4.7 |
| Colon cancer (OD06064) | 24.7 |
| Colon Margin (OD06064) | 12.0 |
| Colon cancer (OD06159) | 1.1 |
| Colon Margin (OD06159) | 6.2 |
| Colon cancer (OD06297-04) | 1.9 |
| Colon Margin (OD06297-05) | 6.9 |
| CC Gr.2 ascend colon (ODO3921) | 0.4 |
| CC Margin (ODO3921) | 2.7 |
| Colon cancer metastasis (OD06104) | 2.4 |
| Lung Margin (OD06104) | 10.2 |
| Colon mets to lung (OD04451-01) | 7.0 |
| Lung Margin (OD04451-02) | 20.4 |
| Normal Prostate | 4.9 |
| Prostate Cancer (OD04410) | 5.9 |
| Prostate Margin (OD04410) | 8.3 |
| Normal Ovary | 1.9 |
| Ovarian cancer (OD06283-03) | 1.2 |
| Ovarian Margin (OD06283-07) | 3.6 |
| Ovarian Cancer 064008 | 7.8 |
| Ovarian cancer (OD06145) | 0.9 |
| Ovarian Margin (OD06145) | 0.9 |
| Ovarian cancer (OD06455-03) | 0.0 |
| Ovarian Margin (OD06455-07) | 7.3 |
| Normal Lung | 14.2 |
| Invasive poor diff. lung adeno (ODO4945-01 | 1.5 |
| Lung Margin (ODO4945-03) | 15.5 |
| Lung Malignant Cancer (OD03126) | 4.2 |
| Lung Margin (OD03126) | 8.3 |
| Lung Cancer (OD05014A) | 5.4 |
| Lung Margin (OD05014B) | 41.5 |
| Lung cancer (OD06081) | 3.8 |
| Lung Margin (OD06081) | 37.6 |
| Lung Cancer (OD04237-01) | 1.6 |
| Lung Margin (OD04237-02) | 33.2 |
| Ocular Melanoma Metastasis | 0.0 |
| Ocular Melanoma Margin (Liver) | 0.0 |
| Melanoma Metastasis | 0.0 |
| Melanoma Margin (Lung) | 37.9 |
| Normal Kidney | 5.5 |
| Kidney Ca, Nuclear grade 2 (OD04338) | 26.6 |
| Kidney Margin (OD04338) | 0.9 |
| Kidney Ca Nuclear grade 1/2 (OD04339) | 2.0 |
| Kidney Margin (OD04339) | 10.3 |
| Kidney Ca, Clear cell type (OD04340) | 4.5 |
| Kidney Margin (OD04340) | 12.6 |
| Kidney Ca, Nuclear grade 3 (OD04348) | 1.4 |
| Kidney Margin (OD04348) | 100.0 |
| Kidney malignant cancer (OD06204B) | 0.0 |
| Kidney normal adjacent tissue (OD06204E) | 7.0 |
| Kidney Cancer (OD04450-01) | 1.2 |
| Kidney Margin (OD04450-03) | 16.6 |
| Kidney Cancer 8120613 | 1.8 |
| Kidney Margin 8120614 | 5.7 |
| Kidney Cancer 9010320 | 0.6 |
| Kidney Margin 9010321 | 2.6 |
| Kidney Cancer 8120607 | 6.2 |
| Kidney Margin 8120608 | 2.3 |
| Normal Uterus | 13.4 |
| Uterine Cancer 064011 | 0.8 |
| Normal Thyroid | 6.1 |
| Thyroid Cancer 064010 | 28.5 |
| Thyroid Cancer A302152 | 46.3 |
| Thyroid Margin A302153 | 21.0 |
| Normal Breast | 10.2 |
| Breast Cancer (OD04566) | 1.5 |
| Breast Cancer 1024 | 4.6 |
| Breast Cancer (OD04590-01) | 62.0 |
| Breast Cancer Mets (OD04590-03) | 98.6 |
| Breast Cancer Metastasis (OD04655-05) | 70.7 |
| Breast Cancer 064006 | 3.6 |
| Breast Cancer 9100266 | 3.4 |
| Breast Margin 9100265 | 2.9 |
| Breast Cancer A209073 | 1.7 |
| Breast Margin A2090734 | 2.5 |
| Breast cancer (OD06083) | 49.7 |
| Breast cancer node metastasis (OD06083) | 64.2 |
| Normal Liver | 0.5 |
| Liver Cancer 1026 | 0.5 |
| Liver Cancer 1025 | 1.8 |
| Liver Cancer 6004-T | 0.0 |
| Liver Tissue 6004-N | 1.3 |
| Liver Cancer 6005-T | 0.5 |
| Liver Tissue 6005-N | 1.4 |
| Liver Cancer 064003 | 0.0 |
| Normal Bladder | 2.8 |
| Bladder Cancer 1023 | 2.5 |
| Bladder Cancer A302173 | 6.2 |
| Normal Stomach | 2.8 |
| Gastric Cancer 9060397 | 0.0 |
| Stomach Margin 9060396 | 1.4 |
| Gastric Cancer 9060395 | 2.3 |
| Stomach Margin 9060394 | 4.1 |
| Gastric Cancer 064005 | 5.7 |

TABLE ABN
Panel 2D

| | Rel. | Rel. | Rel. |
|---|---|---|---|
| | Exp. (%) | Exp. (%) | Exp. (%) |
| | Ag2667, | Ag2767, | Ag2831, |
| | Run | Run | Run |
| Tissue Name | 162558279 | 162555855 | 163578438 |
| Normal Colon | 4.8 | 4.8 | 6.1 |
| CC Well to | 1.1 | 0.8 | 0.9 |
| Mod Diff | | | |
| (ODO3866) | | | |
| CC Margin | 0.8 | 1.2 | 1.5 |
| (ODO3866) | | | |
| CC Gr.2 | 0.8 | 0.5 | 0.3 |
| rectosigmoid | | | |
| (ODO3868) | | | |
| CC Margin | 0.2 | 0.2 | 0.1 |
| (ODO3868) | | | |
| CC Mod Diff | 0.2 | 0.2 | 0.1 |
| (ODO3920) | | | |
| CC Margin | 1.0 | 0.7 | 0.9 |
| (ODO3920) | | | |
| CC Gr.2 | 3.8 | 3.8 | 3.8 |
| ascend | | | |
| colon | | | |
| (ODO3921) | | | |
| CC Margin | 1.4 | 1.5 | 1.0 |
| (ODO3921) | | | |
| CC from | 6.5 | 5.6 | 6.1 |
| Partial | | | |
| Hepatectomy | | | |
| (ODO4309) | | | |
| Mets | | | |
| Liver Margin | 0.3 | 0.4 | 0.1 |
| (ODO4309) | | | |
| Colon mets to | 1.5 | 1.6 | 1.2 |
| lung | | | |
| (OD04451-01) | | | |
| Lung Margin | 3.1 | 4.0 | 3.3 |
| (OD04451-02) | | | |
| Normal | 10.6 | 10.5 | 11.9 |
| Prostate 6546-1 | | | |
| Prostate Cancer | 13.3 | 13.4 | 15.2 |
| (OD04410) | | | |
| Prostate | 8.3 | 12.1 | 10.5 |
| Margin | | | |
| (OD04410) | | | |
| Prostate Cancer | 2.2 | 1.7 | 1.6 |
| (OD04720-01) | | | |
| Prostate | 5.0 | 4.5 | 4.1 |
| Margin | | | |
| (OD04720-02) | | | |
| Normal Lung | 15.0 | 17.3 | 13.5 |
| 061010 | | | |
| Lung Met to | 0.5 | 0.5 | 0.3 |
| Muscle | | | |
| (ODO4286) | | | |
| Muscle Margin | 0.1 | 0.1 | 0.0 |
| (ODO4286) | | | |
| Lung | 3.7 | 3.8 | 3.5 |
| Malignant | | | |
| Cancer | | | |
| (OD03126) | | | |
| Lung Margin | 15.1 | 20.4 | 15.5 |
| (OD03126) | | | |
| Lung Cancer | 4.7 | 4.2 | 2.8 |
| (OD04404) | | | |
| Lung Margin | 12.1 | 12.9 | 9.8 |
| (OD04404) | | | |
| Lung Cancer | 0.6 | 0.7 | 0.4 |
| (OD04565) | | | |
| Lung Margin | 9.9 | 8.4 | 8.6 |
| (OD04565) | | | |
| Lung Cancer | 1.1 | 1.5 | 1.0 |
| (OD04237-01) | | | |
| Lung Margin | 17.4 | 13.0 | 14.3 |
| (OD04237-02) | | | |
| Ocular Mel | 0.0 | 0.1 | 0.0 |
| Met to Liver | | | |
| (ODO4310) | | | |
| Liver Margin | 0.2 | 0.2 | 0.3 |
| (ODO4310) | | | |
| Melanoma | 0.1 | 0.3 | 0.2 |
| Mets to Lung | | | |
| (OD04321) | | | |
| Lung Margin | 21.3 | 20.7 | 19.5 |
| (OD04321) | | | |
| Normal Kidney | 14.9 | 18.4 | 15.2 |
| Kidney Ca, | 0.9 | 1.2 | 0.6 |
| Nuclear grade | | | |
| 2 (OD04338) | | | |
| Kidney Margin | 7.3 | 10.3 | 7.0 |
| (OD04338) | | | |
| Kidney Ca | 0.3 | 0.3 | 0.6 |
| Nuclear grade | | | |
| 1/2 (OD04339) | | | |
| Kidney Margin | 14.8 | 11.7 | 14.6 |
| (OD04339) | | | |
| Kidney Ca, | 6.5 | 7.8 | 8.1 |
| Clear cell type | | | |
| (OD04340) | | | |
| Kidney Margin | 11.0 | 9.2 | 9.8 |
| (OD04340) | | | |
| Kidney Ca, | 1.1 | 0.6 | 1.1 |
| Nuclear grade | | | |
| 3 (OD04348) | | | |
| Kidney Margin | 15.5 | 11.7 | 13.5 |
| (OD04348) | | | |
| Kidney Cancer | 1.7 | 1.2 | 1.6 |
| (OD04622-01) | | | |
| Kidney Margin | 3.0 | 2.7 | 2.5 |
| (OD04622-03) | | | |
| Kidney Cancer | 0.1 | 0.2 | 0.3 |
| (OD04450-01) | | | |
| Kidney Margin | 11.6 | 15.2 | 14.0 |
| (OD04450-03) | | | |
| Kidney Cancer | 2.6 | 2.6 | 2.9 |
| 8120607 | | | |
| Kidney | 2.3 | 2.4 | 2.0 |
| Margin | | | |
| 8120608 | | | |
| Kidney | 8.5 | 9.9 | 9.6 |
| Cancer | | | |
| 8120613 | | | |
| Kidney | 3.0 | 3.2 | 2.8 |
| Margin | | | |
| 8120614 | | | |
| Kidney | 1.0 | 1.6 | 0.9 |
| Cancer | | | |
| 9010320 | | | |
| Kidney | 3.9 | 4.6 | 0.0 |
| Margin | | | |
| 9010321 | | | |
| Normal | 1.3 | 1.1 | 0.6 |
| Uterus | | | |
| Uterus | 1.6 | 1.1 | 1.4 |
| Cancer | | | |
| 064011 | | | |
| Normal | 13.9 | 13.7 | 10.4 |
| Thyroid | | | |
| Thyroid | 33.2 | 35.1 | 36.9 |
| Cancer | | | |
| 064010 | | | |
| Thyroid | 19.3 | 21.3 | 21.5 |
| Cancer | | | |
| A302152 | | | |
| Thyroid | 41.8 | 39.8 | 37.9 |
| Margin | | | |
| A302153 | | | |
| Normal | 1.7 | 2.4 | 1.7 |
| Breast | | | |
| Breast Cancer | 2.0 | 2.2 | 2.5 |
| (OD04566) | | | |
| Breast Cancer | 68.3 | 69.7 | 64.2 |
| (OD04590- | | | |
| 01) | | | |
| Breast Cancer | 100.0 | 100.0 | 100.0 |
| Mets | | | |
| (OD04590- | | | |
| 03) | | | |
| Breast Cancer | 38.7 | 39.0 | 33.4 |
| Metastasis | | | |
| (OD04655- | | | |
| 05) | | | |
| Breast Cancer | 3.7 | 4.0 | 3.9 |
| 064006 | | | |
| Breast Cancer | 2.7 | 2.1 | 2.7 |
| 1024 | | | |
| Breast Cancer | 2.6 | 2.3 | 2.9 |
| 9100266 | | | |
| Breast | 0.9 | 0.8 | 0.6 |
| Margin | | | |
| 9100265 | | | |
| Breast Cancer | 3.5 | 3.7 | 3.8 |
| A209073 | | | |
| Breast | 1.7 | 1.5 | 1.5 |
| Margin | | | |
| A209073 | | | |
| Normal Liver | 0.2 | 0.1 | 0.1 |
| Liver Cancer | 0.0 | 0.1 | 0.2 |
| 064003 | | | |
| Liver Cancer | 0.1 | 0.1 | 0.0 |
| 1025 | | | |
| Liver Cancer | 0.8 | 0.5 | 0.8 |
| 1026 | | | |
| Liver Cancer | 0.1 | 0.1 | 0.1 |
| 6004-T | | | |
| Liver Tissue | 0.8 | 0.9 | 0.9 |
| 6004-N | | | |
| Liver Cancer | 0.4 | 0.7 | 0.5 |
| 6005-T | | | |
| Liver Tissue | 0.1 | 0.1 | 0.1 |
| 6005-N | | | |
| Normal | 3.5 | 3.8 | 4.2 |
| Bladder | | | |
| Bladder | 0.9 | 0.9 | 0.6 |
| Cancer 1023 | | | |
| Bladder | 3.8 | 4.6 | 4.4 |
| Cancer | | | |
| A302173 | | | |
| Bladder | 0.6 | 1.1 | 1.0 |
| Cancer | | | |
| (OD04718- | | | |
| 01) | | | |
| Bladder | 0.6 | 0.3 | 0.8 |
| Normal | | | |
| Adjacent | | | |
| (OD04718- | | | |
| 03) | | | |
| Normal | 0.8 | 0.7 | 0.6 |
| Ovary | | | |
| Ovarian | 7.2 | 9.6 | 8.8 |
| Cancer | | | |
| 064008 | | | |
| Ovarian | 0.2 | 0.2 | 0.1 |
| Cancer | | | |
| (OD04768- | | | |
| 07) | | | |
| Ovary Margin | 1.5 | 1.8 | 1.3 |
| (OD04768- | | | |
| 08) | | | |
| Normal | 0.5 | 1.0 | 0.6 |
| Stomach | | | |
| Gastric | 0.3 | 0.2 | 0.6 |
| Cancer | | | |
| 9060358 | | | |
| Stomach | 0.4 | 0.9 | 0.8 |
| Margin | | | |
| 9060359 | | | |
| Gastric | 1.6 | 2.2 | 1.3 |
| Cancer | | | |
| 9060395 | | | |
| Stomach | 0.7 | 1.2 | 0.6 |
| Margin | | | |
| 9060394 | | | |
| Gastric | 0.3 | 0.3 | 0.0 |
| Cancer | | | |
| 9060397 | | | |
| Stomach | 0.3 | 0.3 | 0.0 |
| Margin | | | |
| 9060396 | | | |
| Gastric | 11.7 | 16.5 | 11.6 |
| Cancer | | | |
| 064005 | | | |

TABLE ABO
Panel 3D

| | Rel. Exp. (%) |
|---|---|
| | Ag2831, Run |
| Tissue Name | 164843468 |
| Daoy- Medulloblastoma | 0.4 |
| TE671- Medulloblastoma | 3.7 |
| D283 Med- Medulloblastoma | 6.7 |
| PFSK-1- Primitive Neuroectodermal | 0.0 |
| XF-498- CNS | 1.2 |
| SNB-78- Glioma | 1.7 |
| SF-268- Glioblastoma | 0.0 |
| T98G- Glioblastoma | 0.0 |
| SK-N-SH- Neuroblastoma (metastasis) | 0.0 |
| SF-295- Glioblastoma | 0.0 |
| Cerebellum | 0.0 |
| Cerebellum | 0.0 |
| NCI-H292- Mucoepidermoid lung carcinoma | 23.7 |
| DMS-114- Small cell lung cancer | 0.0 |
| DMS-79- Small cell lung cancer | 1.1 |
| NCI-H146- Small cell lung cancer | 100.0 |
| NCI-H526- Small cell lung cancer | 5.6 |
| NCI-N417- Small cell lung cancer | 0.8 |
| NCI-H82- Small cell lung cancer | 0.0 |
| NCI-H157- Squamous cell lung cancer (metastasis) | 0.0 |
| NCI-H1155- Large cell lung cancer | 14.6 |
| NCI-H1299- Large cell lung cancer | 0.0 |
| NC1-H727- Lung carcinoid | 14.8 |
| NCI-UMC-11- Lung carcinoid | 84.1 |
| LX-1- Small cell lung cancer | 7.5 |
| Colo-205- Colon cancer | 18.7 |
| KM12- Colon cancer | 66.4 |
| KM20L2- Colon cancer | 8.4 |
| NCI-H716- Colon cancer | 23.2 |
| SW-48- Colon adenocarcinoma | 63.7 |
| SW1116- Colon adenocarcinoma | 15.5 |
| LS 174T- Colon adenocarcinoma | 62.9 |
| SW-948- Colon adenocarcinoma | 2.7 |
| SW-480- Colon adenocarcinoma | 39.2 |
| NCI-SNU-5- Gastric carcinoma | 0.0 |
| KATO III- Gastric carcinoma | 33.0 |
| NCI-SNU-16- Gastric carcinoma | 0.0 |
| NCI-SNU-1- Gastric carcinoma | 35.6 |
| RF-1- Gastric adenocarcinoma | 0.0 |
| RF-48- Gastric adenocarcinoma | 0.7 |
| MKN-45- Gastric carcinoma | 96.6 |
| NCI-N87- Gastric carcinoma | 79.6 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma (metastasis) | 9.4 |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.7 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.6 |
| 769-P- Clear cell renal carcinoma | 3.2 |
| Caki-2- Clear cell renal carcinoma | 0.8 |
| SW 839- Clear cell renal carcinoma | 0.9 |
| G401- Wilms' tumor | 0.0 |
| Hs766T- Pancreatic carcinoma (LN metastasis) | 0.0 |
| CAPAN-1- Pancreatic adenocarcinoma | 0.0 |
| (liver metastasis) | |
| SU86.86- Pancreatic carcinoma (liver metastasis) | 0.0 |
| BxPC-3- Pancreatic adenocarcinoma | 0.0 |
| HPAC- Pancreatic adenocarcinoma | 0.0 |
| MIA PaCa-2- Pancreatic carcinoma | 0.0 |
| CFPAC-1- Pancreatic ductal adenocarcinoma | 0.0 |
| PANC-1- Pancreatic epithelioid ductal carcinoma | 0.6 |
| T24- Bladder carcinma (transitional cell) | 0.0 |
| 5637- Bladder carcinoma | 0.0 |
| HT-1197- Bladder carcinoma | 3.2 |
| UM-UC-3- Bladder carcinma (transitional cell) | 0.0 |
| A204- Rhabdomyosarcoma | 0.0 |
| HT-1080- Fibrosarcoma | 0.0 |
| MG-63- Osteosarcoma | 0.0 |
| SK-LMS-1- Leiomyosarcoma (vulva) | 0.0 |
| SJRH30- Rhabdomyosarcoma (met to bone marrow) | 27.7 |
| A431- Epidermoid carcinoma | 17.8 |
| WM266-4- Melanoma | 0.0 |
| DU 145- Prostate carcinoma (brain metastasis) | 0.0 |
| MDA-MB-468- Breast adenocarcinoma | 2.7 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 1.2 |

TABLE ABP
Panel 4.1D

| | Rel. Exp. (%) | Rel. Exp. (%) |
|---|---|---|
| | Ag2831, Run | Ag5124, Run |
| Tissue Name | 244570230 | 225784387 |
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 | 0.0 | 0.0 |
| lymphocyte act | | |
| CD45RO CD4 | 0.0 | 0.0 |
| lymphocyte act | | |
| CD8 lymphocyte act | 0.0 | 0.0 |
| Secondary CD8 | 0.0 | 0.0 |
| lymphocyte rest | | |
| Secondary CD8 | 0.0 | 0.0 |
| lymphocyte act | | |
| CD4 lymphocyte none | 0.0 | 0.0 |
| 2ry Th1/Th2/Tr1_anti- | 0.0 | 0.0 |
| CD95 CH11 | | |
| LAK cells rest | 0.0 | 0.0 |
| LAK cells 1L-2 | 0.0 | 0.0 |
| LAK cells IL-2 + IL-12 | 0.0 | 0.0 |
| LAK cells IL-2 + IFN | 0.0 | 0.0 |
| gamma | | |
| LAK cells IL-2 + IL-18 | 0.0 | 0.0 |
| LAK cells | 0.0 | 0.0 |
| PMA/ionomycin | | |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes | 1.3 | 0.0 |
| CD40L and IL-4 | | |
| EOL-1 dbcAMP | 0.0 | 0.0 |
| EOL-1 dbcAMP | 0.0 | 0.0 |
| PMA/ionomycin | | |
| Dendritic cells none | 0.0 | 0.0 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 0.0 |
| Macrophages rest | 0.0 | 0.0 |
| Macrophages LPS | 0.0 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 9.0 |
| HUVEC TNF alpha + | 0.0 | 0.0 |
| IFN gamma | | |
| HUVEC TNF alpha + IL4 | 0.0 | 0.0 |
| HUVEC IL-11 | 0.0 | 0.0 |
| Lung Microvascular | 0.0 | 0.0 |
| EC none | | |
| Lung Microvascular | 0.0 | 0.0 |
| EC TNFalpha + IL-1beta | | |
| Microvascular Dermal | 0.0 | 0.0 |
| EC none | | |
| Microsvasular Dermal | 0.0 | 0.0 |
| EC TNFalpha + IL-1beta | | |
| Bronchial epithelium | 0.0 | 0.0 |
| TNFalpha + IL1 beta | | |
| Small airway | 0.0 | 0.0 |
| epithelium none | | |
| Small airway epithelium | 11.5 | 0.0 |
| TNFalpha + IL-1beta | | |
| Coronery artery SMC rest | 0.0 | 0.0 |
| Coronery artery SMC | 0.0 | 0.0 |
| TNFalpha + IL-1beta | | |
| Astrocytes rest | 0.9 | 0.0 |
| Astrocytes TNFalpha + | 9.9 | 0.0 |
| IL-1beta | | |
| KU-812 (Basophil) rest | 0.0 | 0.0 |
| KU-812 (Basophil) | 0.0 | 0.0 |
| PMA/ionomycin | | |
| CCD1106 | 0.8 | 0.0 |
| (Keratinocytes) none | | |
| CCD1106 (Keratinocytes) | 4.5 | 0.0 |
| TNFalpha + IL-1beta | | |
| Liver cirrhosis | 14.9 | 19.9 |
| NC1-H292 none | 19.9 | 0.0 |
| NC1-H292 IL-4 | 62.4 | 0.0 |
| NCI-H292 IL-9 | 57.8 | 0.0 |
| NCI-H292 IL-13 | 73.2 | 0.0 |
| NCI-H292 IFN gamma | 21.0 | 0.0 |
| HPAEC none | 0.0 | 0.0 |
| HPAEC TNF alpha + | 3.6 | 0.0 |
| IL-1 beta | | |
| Lung fibroblast none | 17.0 | 0.0 |
| Lung fibroblast TNF | 0.0 | 0.0 |
| alpha + IL-1 beta | | |
| Lung fibroblast IL-4 | 9.7 | 48.3 |
| Lung fibroblast IL-9 | 6.7 | 0.0 |
| Lung fibroblast IL-13 | 1.6 | 17.1 |
| Lung fibroblast IFN gamma | 21.3 | 26.2 |
| Dermal fibroblast | 0.0 | 0.0 |
| CCD1070 rest | | |
| Dermal fibroblast | 0.0 | 0.0 |
| CCD1070 TNF alpha | | |
| Dermal fibroblast | 0.0 | 0.0 |
| CCD1070 IL-1 beta | | |
| Dermal fibroblast 1FN | 0.0 | 0.0 |
| gamma | | |
| Dermal fibroblast IL-4 | 0.0 | 0.0 |
| Dermal Fibroblasts rest | 0.0 | 0.0 |
| Neutrophils TNFa + LPS | 0.0 | 0.0 |
| Neutrophils rest | 0.0 | 0.0 |
| Colon | 2.2 | 8.7 |
| Lung | 2.1 | 100.0 |
| Thymus | 1.8 | 0.0 |
| Kidney | 100.0 | 36.9 |

TABLE ABQ
Panel 4D

| | Rel. Exp. (%) | Rel. Exp. (%) | Rel. Exp. (%) | Rel. Exp. (%) |
|---|---|---|---|---|
| | Ag2505, Run | Ag2667, Run | Ag2767, Run | Ag2831, Run |
| Tissue Name | 164318134 | 158912431 | 162015289 | 162350949 |
| Secondary Th1 act | 0.0 | 0.0 | 0.0 | 0.0 |
| Secondary Th2 act | 0.0 | 0.0 | 0.0 | 0.0 |
| Secondary Tr1 act | 0.2 | 0.0 | 0.0 | 0.0 |
| Secondary Th1 rest | 0.0 | 0.0 | 0.0 | 0.0 |
| Secondary Th2 rest | 0.3 | 0.0 | 0.0 | 0.0 |
| Secondary Tr1 rest | 0.0 | 0.0 | 0.0 | 0.4 |
| Primary Th1 act | 0.0 | 0.0 | 0.0 | 0.0 |
| Primary Th2 act | 0.2 | 0.0 | 0.0 | 0.0 |
| Primary Tr1 act | 0.1 | 0.0 | 0.0 | 0.0 |
| Primary Th1 rest | 0.1 | 0.0 | 0.0 | 0.4 |
| Primary Th2 rest | 0.0 | 0.0 | 0.0 | 0.0 |
| Primary Tr1 rest | 0.1 | 0.0 | 0.3 | 0.0 |
| CD45RA CD4 lymphocyte act | 0.0 | 0.0 | 0.0 | 0.0 |
| CD45RO CD4 lymphocyte act | 0.0 | 0.0 | 0.0 | 0.0 |
| CD8 lymphocyte act | 0.0 | 0.0 | 0.0 | 0.0 |
| Secondary CD8 | 0.0 | 0.0 | 0.0 | 0.0 |
| lymphocyte rest | | | | |
| Secondary CD8 | 0.0 | 0.0 | 0.0 | 0.0 |
| lymphocyte act | | | | |
| CD4 lymphocyte none | 0.0 | 0.0 | 0.0 | 0.0 |
| 2ry Th1/Th2/Tr1_{—} | 0.0 | 0.0 | 0.0 | 0.0 |
| anti-CD95 CH11 | | | | |
| LAK cells rest | 0.0 | 0.0 | 0.0 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN | 0.0 | 0.0 | 0.0 | 0.0 |
| gamma | | | | |
| LAK cells IL-2 + IL-18 | 0.0 | 0.0 | 0.0 | 0.0 |
| LAK cells PMA/ionomycin | 0.0 | 0.0 | 0.0 | 0.0 |
| NK Cells IL-2 rest | 0.0 | 0.0 | 0.0 | 0.0 |
| Two Way MLR 3 day | 0.0 | 0.0 | 0.0 | 0.0 |
| Two Way MLR 5 day | 0.0 | 0.0 | 0.0 | 0.0 |
| Two Way MLR 7 day | 0.0 | 0.0 | 0.0 | 0.0 |
| PBMC rest | 0.0 | 0.0 | 0.0 | 0.0 |
| PBMC PWM | 0.1 | 0.0 | 0.0 | 0.0 |
| PBMC PHA-L | 0.0 | 0.6 | 0.0 | 0.0 |
| Ramos (B cell) none | 0.0 | 0.0 | 0.0 | 0.0 |
| Ramos (B cell) ionomycin | 0.0 | 0.3 | 0.0 | 0.0 |
| B lymphocytes PWM | 0.2 | 0.3 | 0.9 | 0.7 |
| B lymphocytes CD40L | 0.1 | 0.7 | 0.0 | 0.0 |
| and IL-4 | | | | |
| EOL-1 dbcAMP | 0.0 | 0.0 | 0.0 | 0.0 |
| EOL-1 dbcAMP | 0.0 | 0.0 | 0.0 | 0.0 |
| PMA/ionomycin | | | | |
| Dendritic cells none | 0.1 | 0.0 | 0.0 | 0.3 |
| Dendritic cells LPS | 0.0 | 0.0 | 0.0 | 0.0 |
| Dendritic cells | 0.0 | 0.0 | 0.0 | 0.0 |
| anti-CD40 | | | | |
| Monocytes rest | 0.0 | 0.0 | 0.0 | 0.0 |
| Monocytes LPS | 0.0 | 0.0 | 0.0 | 0.0 |
| Macrophages rest | 0.0 | 0.0 | 0.3 | 0.0 |
| Macrophages LPS | 0.0 | 0.0 | 0.0 | 0.0 |
| HUVEC none | 0.0 | 0.0 | 0.0 | 0.0 |
| HUVEC starved | 0.3 | 0.0 | 0.0 | 0.0 |
| HUVEC IL-1beta | 0.2 | 0.3 | 0.0 | 0.0 |
| HUVEC IFN gamma | 0.1 | 0.0 | 0.0 | 0.0 |
| HUVEC TNF alpha + IFN | 0.0 | 0.0 | 0.0 | 0.7 |
| gamma | | | | |
| HUVEC TNF alpha + IL4 | 0.1 | 0.0 | 0.0 | 0.0 |
| HUVEC IL-11 | 0.0 | 0.0 | 0.0 | 0.0 |
| Lung Microvascular EC | 0.0 | 0.0 | 0.0 | 0.0 |
| none | | | | |
| Lung Microvascular EC | 0.0 | 0.3 | 0.0 | 0.0 |
| TNFalpha + IL-1beta | | | | |
| Microvascular Dermal EC | 0.0 | 0.0 | 0.0 | 0.0 |
| none | | | | |
| Microsvasular Dermal EC | 0.2 | 0.0 | 1.4 | 0.0 |
| TNFalpha + IL-1beta | | | | |
| Bronchial epithelium | 2.0 | 0.0 | 1.2 | 0.0 |
| TNFalpha + IL1beta | | | | |
| Small airway epithelium | 0.5 | 0.0 | 0.0 | 0.4 |
| none | | | | |
| Small airway epithelium | 19.6 | 10.4 | 11.7 | 8.4 |
| TNFalpha + IL-1beta | | | | |
| Coronery artery SMC rest | 0.0 | 0.0 | 0.0 | 0.0 |
| Coronery artery SMC | 0.0 | 0.0 | 0.0 | 0.0 |
| TNFalpha + IL-1beta | | | | |
| Astrocytes rest | 2.0 | 2.6 | 1.9 | 2.3 |
| Astrocytes TNFalpha + | 2.0 | 4.6 | 8.2 | 4.4 |
| IL-1beta | | | | |
| KU-812 (Basophil) rest | 0.0 | 0.0 | 0.0 | 0.0 |
| KU-812 (Basophil) | 0.0 | 0.3 | 0.0 | 0.0 |
| PMA/ionomycin | | | | |
| CCD1106 (Keratinocytes) | 0.4 | 0.4 | 0.8 | 0.6 |
| none | | | | |
| CCD1106 (Keratinocytes) | 1.3 | 0.0 | 2.0 | 1.5 |
| TNFalpha + IL-1beta | | | | |
| Liver cirrhosis | 7.5 | 3.4 | 8.0 | 4.7 |
| Lupus kidney | 13.3 | 6.5 | 12.2 | 5.4 |
| NCI-H292 none | 21.8 | 11.0 | 14.9 | 15.5 |
| NCI-H292 IL-4 | 42.0 | 36.1 | 43.5 | 44.4 |
| NCI-H292 IL-9 | 41.8 | 48.3 | 32.8 | 28.1 |
| NCI-H292 IL-13 | 20.9 | 17.2 | 30.4 | 21.6 |
| NCI-H292 IFN gamma | 14.4 | 12.9 | 14.6 | 10.2 |
| HPAEC none | 0.0 | 0.0 | 0.0 | 0.0 |
| HPAEC TNF alpha + IL-1 | 0.5 | 0.9 | 1.1 | 1.0 |
| beta | | | | |
| Lung fibroblast none | 4.5 | 2.4 | 2.6 | 4.5 |
| Lung fibroblast TNF | 0.3 | 0.2 | 0.0 | 0.0 |
| alpha + IL-1 beta | | | | |
| Lung fibroblast IL-4 | 14.6 | 6.3 | 9.0 | 8.8 |
| Lung fibroblast IL-9 | 3.9 | 1.0 | 8.6 | 2.9 |
| Lung fibroblast IL-13 | 8.7 | 5.6 | 5.9 | 3.5 |
| Lung fibroblast IFN | 14.9 | 3.8 | 6.7 | 4.5 |
| gamma | | | | |
| Dermal fibroblast | 0.0 | 0.0 | 0.0 | 0.0 |
| CCD1070 rest | | | | |
| Dermal fibroblast | 0.0 | 0.0 | 0.0 | 0.0 |
| CCD1070 TNF alpha | | | | |
| Dermal fibroblast | 0.0 | 0.0 | 0.0 | 0.0 |
| CCD1070 IL-1 beta | | | | |
| Dermal fibroblast IFN | 0.0 | 0.0 | 0.0 | 0.0 |
| gamma | | | | |
| Dermal fibroblast IL-4 | 0.2 | 0.8 | 0.8 | 0.0 |
| IBD Colitis 2 | 0.3 | 0.0 | 0.4 | 0.4 |
| IBD Crohn's | 9.9 | 4.1 | 2.7 | 3.6 |
| Colon | 41.2 | 20.9 | 27.5 | 20.0 |
| Lung | 61.6 | 35.1 | 34.6 | 35.4 |
| Thymus | 100.0 | 100.0 | 100.0 | 100.0 |
| Kidney | 21.3 | 13.9 | 14.9 | 14.0 |

TABLE ABR
Panel 5 Islet

| | Rel. Exp. (%) |
|---|---|
| | Ag2505, Run |
| Tissue Name | 248045752 |
| 97457_Patient-02go_adipose | 32.3 |
| 97476_Patient-07sk_skeletal muscle | 8.2 |
| 97477_Patient-07ut_uterus | 31.4 |
| 97478_Patient-07pl_placenta | 3.5 |
| 99167_Bayer Patient 1 | 9.5 |
| 97482_Patient-08ut_uterus | 90.1 |
| 97483_Patient-08pl_placenta | 7.4 |
| 97486_Patient-09sk_skeletal muscle | 1.4 |
| 97487_Patient-09ut_uterus | 78.5 |
| 97488_Patient-09pl_placenta | 0.6 |
| 97492_Patient-10ut_uterus | 66.0 |
| 97493_Patient-10pl_placenta | 3.1 |
| 97495_Patient-11go_adipose | 28.3 |
| 97496_Patient-11sk_skeletal muscle | 5.8 |
| 97497_Patient-11ut uterus | 35.4 |
| 97498_Patient-11pl_placenta | 2.0 |
| 97500_Patient-12go_adipose | 35.1 |
| 97501_Patient-12sk_skeletal muscle | 9.9 |
| 97502_Patient-12ut_uterus | 100.0 |
| 97503_Patient-12pl_placenta | 4.1 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.2 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 21.2 |
| 73556 Heart_Cardiac stromal cells (primary) | 0.0 |
| 81735_Small Intestine | 36.9 |
| 72409_Kidney_Proximal Convoluted Tubule | 4.6 |
| 82685_Small intestine_Duodenum | 27.0 |
| 90650_Adrenal_Adrenocortical adenoma | 0.3 |
| 72410_Kidney_HRCE | 7.2 |
| 72411_Kidney_HRE | 10.7 |
| 73139_Uterus_Uterine smooth muscle cells | 0.0 |

TABLE ABS
general oncology screening panel_v_2.4

| | Rel. Exp. (%) | Rel. Exp. (%) | Rel. Exp. (%) |
|---|---|---|---|
| | Ag2505, Run | Ag5113, Run | Ag5124, Run |
| Tissue Name | 267145080 | 260280405 | 259936347 |
| Colon cancer 1 | 7.7 | 3.7 | 9.3 |
| Colon NAT 1 | 2.5 | 1.3 | 3.6 |
| Colon cancer 2 | 26.4 | 0.9 | 9.5 |
| Colon NAT 2 | 8.4 | 2.8 | 0.0 |
| Colon cancer 3 | 38.2 | 5.1 | 20.0 |
| Colon NAT 3 | 16.7 | 14.0 | 18.6 |
| Colon malignant | 69.7 | 1.9 | 20.3 |
| cancer 4 | | | |
| Colon NAT 4 | 8.7 | 8.7 | 4.4 |
| Lung cancer 1 | 7.2 | ^{2.4} | ^{5.3} |
| Lung NAT 1 | 8.0 | 6.6 | 16.5 |
| Lung cancer 2 | 26.1 | 8.4 | 67.8 |
| Lung NAT 2 | 17.4 | 15.2 | 69.3 |
| Squamous cell | 19.6 | 12.8 | 76.8 |
| carcinoma 3 | | | |
| Lung NAT 3 | 8.3 | 2.6 | 0.0 |
| Metastatic melanoma 1 | 14.5 | 11.5 | 36.1 |
| Melanoma 2 | 1.1 | 0.0 | 0.0 |
| Melanoma 3 | 2.5 | 0.8 | 1.4 |
| Metastatic melanoma 4 | 13.5 | 7.3 | 100.0 |
| Metastatic melanoma 5 | 12.6 | 11.0 | 99.3 |
| Bladder cancer 1 | 1.0 | 2.0 | 0.0 |
| Bladder NAT 1 | 0.0 | 0.0 | 0.0 |
| Bladder cancer 2 | 2.4 | 3.5 | 6.0 |
| Bladder NAT 2 | 0.3 | 0.5 | 0.0 |
| Bladder NAT 3 | 0.7 | 0.0 | 0.0 |
| Bladder NAT 4 | 1.5 | 0.0 | 12.5 |
| Prostate | 100.0 | 100.0 | 0.0 |
| adenocarcinoma 1 | | | |
| Prostate | 12.9 | 5.6 | 2.8 |
| adenocarcinoma 2 | | | |
| Prostate | 15.1 | 1.7 | 6.8 |
| adenocarcinoma 3 | | | |
| Prostate | 15.9 | 2.1 | 18.2 |
| adenocarcinoma 4 | | | |
| Prostate NAT 5 | 14.7 | 2.3 | 0.0 |
| Prostate | 13.1 | 2.0 | 9.2 |
| adenocarcinoma 6 | | | |
| Prostate | 16.2 | 8.1 | 35.6 |
| adenocarcinoma 7 | | | |
| Prostate | 4.5 | 2.3 | 0.0 |
| adenocarcinoma 8 | | | |
| Prostate | 33.4 | 21.0 | 69.3 |
| adenocarcinoma 9 | | | |
| Prostate NAT 10 | 7.7 | 0.9 | 0.0 |
| Kidney cancer 1 | 9.6 | 1.1 | 18.0 |
| Kidney NAT 1 | 33.4 | 4.0 | 27.4 |
| Kidney cancer 2 | 19.8 | 6.0 | 65.1 |
| Kidney NAT 2 | 64.6 | 10.2 | 31.6 |
| Kidney cancer 3 | 7.1 | 1.6 | 2.2 |
| Kidney NAT 3 | 29.7 | 1.7 | 12.7 |
| Kidney cancer 4 | 8.5 | 5.2 | 12.8 |
| Kidney NAT 4 | 7.9 | 2.1 | 26.6 |

AI_comprehensive panel_v1.0 Summary: Ag2505/Ag2831 Two experiments with different probes and primer sets are in excellent agreement, with highest expression of this gene seen in rheumatoid arthritis bone (CT=27-29). This gene shows ubiquitous expression, but expression of this gene is higher in bone, synovium, cartilage and synovial fluid from RA patients as compared to expression in samples from OA patients, normal and diseased lung. Expression of this gene is downregulated in Crohn's samples as compared to the corresponding control samples. This gene encode a putative novel adhesion molecule which is homologous to mouse POEM (preosteoblast epidermal growth factor-like repeat protein with meprin) or nephronectin. Murine nephronectin may function in multiple biological processes including development of the kidney (1) and bone (2) and contribute to liver and lung fibrosis (3). Therefore, therapeutic modulation of this gene may be useful in the treatment of autoimmune and inflammatory diseases such as rheumatoid and osteoarthritis, Inflammatory bowel disease, COPD, asthma, psoriasis, liver and lung fibrosis.

### REFERENCES

1. Miner J H. J Cell Biol Jul. 23, 2001;154(2):257-9, PMID: 11470814.

2. Morimura N et al., 2001 J. Biol. Chem. Nov. 9, 2000;276(45):42172-42181, PMID: 11546798.

3. Levine et al., 2000, Am J Pathol June 2000;156(6):1927-35, PMID: 10854216.

CNS_neurodegeneration_v1.0 Summary: Ag2505/Ag2667/Ag2767/Ag2831/Ag7237 Six experiments with three different probe and primer sets are in excellent agreement. This panel confirms the expression of this gene at low levels in the brain in an independent group of individuals. This gene is found to be upregulated in the temporal cortex of Alzheimer's disease patients. This gene codes for a homolog of mouse POEM (Nephronectin short isoform), a cell adhesion molecule with EGF domains. Alpha secretase activity, which is generally believed to be a beneficial processing alternative to beta secretase, is increased by EGF in neuronal cells (1). This suggests the increased expression of this gene observed here is a compensatory action in the brain to counter the mechanisms of Alzheimer's Disease. Therefore, the protein encoded by this gene may be a potential therapeutic agent for the treatment of Alzheimer's disease and other neurodegenerative diseases.

EGF is also known to facilitate long term potentiation (LTP) in the hippocampus, a process thought to underlie learning and memory (2). Therefore, this gene may have utility in treating disorders of memory, such as neurodegenerative diseases and aging, when used alone or in combination with other growth factors such as bFGF.

In addition, EGF supports the growth and differentiation of dopaminergic neurons (3), which are selectively vulnerable to loss in Parkinson's disease. Therefore, this gene product may have utility in treating Parkinson's Disease.

Ag5113 Expression of this gene is low/undetectable (CTs>35) across all of the samples on this panel (data not shown).

### REFERENCES

1. Slack B E, Breu J, Muchnicki L, Wurtman R J, 1997, Biochem J 327 (Pt 1):245-9.

2. Abe K, Ishiyama J, Saito H, 1992, Brain Res 593(2):335-8.

3. Storch A, Paul G, Csete M, Boehin B O, Carvey P M, Kupsch A, Schwarz J, 2001, Exp Neurol 170(2):317-25.

General_screening_panel_v1.5 Summary: Ag5113/Ag5124 Highest expression of this gene is detected in fetal lung (CT=29). Low but significant expression of this gene is also seen in tissues with metabolic function including adipose, pancreas, and gastrointestinal tract. See panel 1.3 for further discussion of this gene.

General_screening_panel_v1.6 Summary: Ag7237 Highest expression of this gene is detected in fetal lung (CT=27). Expression of this gene is higher in fetal (CTs=27-33) as compared to corresponding adult lung, kidney, liver and skeletal muscle tissues (CT=32-40). Therefore, expression of this gene may be used to distinguish between these fetal and adult tissues. In addition, the relative overexpression of this gene in fetal tissue suggests that the protein product may enhance growth or development of these tissues in the fetus and thus may also act in a regenerative capacity in the adult. Therefore, therapeutic modulation of the protein encoded by this gene could be useful in treatment of lung, liver, kidney and muscle related diseases.

Moderate to low levels of expression of this gene is also seen in cancer cell lines derived from squamous cell carcinoma, brain, colon, renal, lung, breast, and ovarian cancers. Therefore, expression of this gene may be useful as diagnostic marker for detection of these cancers. Furthermore, therapeutic modulation of this gene may be useful in the treatment of squamous cell carcinoma, brain, colon, renal, lung, breast, and ovarian cancers.

Moderate to low levels of expression of this gene is also seen in tissues with metabolic/endocrine functions and also in all the regions of brain. See panel 1.3D for further discussion of this gene.

Panel 1.3D Summary: Ag2505/Ag2667/Ag2767/Ag2831 Four experiments with two different probes and primer sets are in good agreement. Highest expression of this gene is detected in the thyroid and fetal lung (CTs=29-31). Moderate to low levels of expression of this gene is also seen in other tissues with metabolic/endocrine functions, including skeletal muscle, fetal skeletal muscle, small intestine, stomach, pancreas, adipose and fetal heart. Very low levels are also seen in heart and placenta. Nephronectin is the ligand for the alpha8beta1 integrin as evidenced by two independent sets of published data (1,4). Integrins are known to mediate development and organogenesis (5,6). Nephronectin can bind integrins including alpha5beta3, alpha5beta5, alpha5beta6 and alpha4beta7, but not alpha4beta1, alpha3beta1, alpha2beta1 or alpha1beta1. Nephronectin interacts with integrins via the RGD sequence, but RGD alone is not sufficient for binding, the MAM domain is also required (2). MAM domains are thought to have an adhesive function. Thus, modulation of the expression or activity of this gene product by protein or antibody therapeutics may be an effective therapeutic for disorders involving alpha8beta1 integrin signaling including inflammatory diseases.

Obesity has also been linked as an inflammatory condition (7) and thus humanized antibodies may also be therapeutically relevant in treating this condition and related complications such as type II diabetes.

Overall, this gene is expressed at a low to moderate level in the normal tissues on this panel. Furthermore, the brain, prostate, lung and colon cancer cell lines show a very low level of expression compared to the normal organs. This suggests that this molecule can potentialy be used as a therapeutic inhibitor for these cancers.

Moderate to low levels of expression is seen in all the regions of the central nervous system including substantia nigra, hippocampus, cortex, amygdala, thalamus and spinal cord. POEM is a ligand for alpha8beta1 integrin, which in turn promotes attachment, cell spreading, and neurite outgrowth on fibronectin (8). See CNS_neurodegeneration_v1.0 for discussion of this gene in the central nervous system.

### REFERENCE

4. Brandenberger R et al., 2001, J Cell Biol 154(2):447-58, PMID: 11470831.

5. Schwartz et al., 1995, Annu. Rev. Cell Dev. Biol. 11, 549-599, PMID: 8689569.

6. Clark and Brugge, 1995, Science 268, 233-239, PMID: 7716514.

7. Das U N, 2001, Nutrition 17(11-12):953-66, PMID: 11744348.

8. Muller et al., 1995, Mol Biol Cell 6(4):433-48, PMID: 7626807

Panel 2.2 Summary: Ag2831 Highest expression of this gene is detected in kidney (CT=30.3). Expression of this gene is down regulated in kidney, lung and colon cancer as compared to the corresponding normal adjacent tissue. Conversely, increased expression of this gene is seen in breast cancer samples. Therefore, expression of this gene may be used to distinguish between cancer and normal kidney, lung, colon and breast. In addition, therapeutic modulation of this gene or its protein product in the form of protein therapeutic or through the use of antibodies may be useful in the treatment of kidney, lung, colon and breast cancer.

Panel 2D Summary: Ag2667/Ag2767/Ag2831 Three experiments with same probe and primer sets are in excellent agreement, with highest expression of this gene in metastatic breast cancer sample (CTs=26). Expression of this gene in this panel correlates with the expression pattern seen in panel 2.2. See panel 2.2 for further discussion of this gene.

Panel 3D Summary: Ag2831 Highest expression of this gene is detected in a small cell lung cancer NCI-H146 cell line (CT=29.7). Moderate to low levels of expression of this gene is also seen in cancer cell lines derived from epidermoid carcinoma, rhabodomyosacoma, gastric, colon and small cell lung cancers. Therefore, expression of this gene may be used as diagnostic marker for detection of these cancers. Furthermore, therapeutic modulation of this gene or its protein product through the use of antibodies may be useful in the treatment of these cancers.

Panel 4.1D Summary: Ag2831 Highest expression of this gene is detected in kidney (CT=31.3). In addition, moderate to low levels of expression of this gene is mainly seen in lung fibroblasts, and mucoepidermoid NCI-H292 cells. Expression of this gene is upregulated in cytokine treated NCI-H292 cells, small airway epithelium and astrocytes. This expression pattern correlates with the expression observed in panel 4D. See panel 4D and AI panel for further discussion of this gene.

Ag5113/Ag5124 Highest expression of this gene is seen in lung (CT=33). Low levels of expression of this gene is also seen in kidney and IL-4 treated lung fibroblasts.

Panel 4D Summary: Ag2505 Highest expression of this transcript is found in the thymus and the lung(CTs=27-28). Consistent with this lung expression, this transcript is found in the pulmonary mucoepidermoid cell line H292 and is up-regulated upon treatment with the Th2 cytokines IL4 and IL9. This gene is also expressed at lower levels in lung fibroblasts treated with IL4. This transcript profile suggests that modulation of the expression or activity of this gene product by protein or antibody therapeutics may be beneficial for the treatment of inflammatory lung diseases such as asthma, emphysema and chronic obstructive pulmonary diseases.

Furthermore, therapeutics designed with the protein encoded for by this transcript could be important for maintaining or restoring normal function of thymus during inflammation.

Panel 5 Islet Summary: Ag2505 Highest expression of this gene is detected in uterus (CT=30). Moderate expression of this gene is also seen in adipose and skeletal muscle of gestational diabetic patients requiring(and not requiring daily injections of insulin. This gene is also expressed in samples derived from pregnant and a nondiabetic, but overweight patient. In addition, this gene is also expressed in islet beta cells (those that are insulin producing) and small intestine. Therefore, therapeutic modulation of this gene may be useful in the treatment of metabolically related diseases including obesity, Type I and Type II diabetes.

general oncology screening panel_v 2.4 Summary: Ag2505 Highest expression of this gene is detected in prostate cancer (CT=27.7). Moderate to low levels of expression of this gene is seen in both normal and cancer samples derived from colon, lung, prostate and kidney. As Consistent with panels 2.2 and 2D, expression of this gene is downregulated in kidney cancer as compared to normal kidney. But higher expression of this gene is seen in colon cancer as compared to corresponding normal adjacent sample. Therefore, expression of this gene may be used to distinguish between cancer and normal kidney and colon tissue. See panel 1.3, 1.6, 2.2 for further discussion of this gene.

Ag5113/Ag5124 Highest expression of this gene is seen in metastatic melanoma and prostate cancer (CTs=31-33.7). Significant expression of this gene is seen in cancer samples derived from kidney, lung, and prostate cancers.

AC. CG51264-01, CG51264-06 and CG51264-07: ST7-Like Protein (17941787).

Expression of gene CG51264-01, CG51264-06 and CG51264-07 was assessed using the primer-probe set Ag7547, described in Table ACA. Results of the RTQ-PCR runs are shown in Table ACB.

TABLE ACA

Probe Name Ag7547

| | | | | |
|---|---|---|---|---|
| Primers | Sequences | Length | Start Position | SEQ ID No |

| | | | | |
|---|---|---|---|---|
| Forward | 5′-agcattgggatgtacttgtaagc- | 23 | 1592 | 363 |
| | 3′ | | | |
| Probe | TET-5′- | 29 | 1630 | 364 |
| | ctgtgtttcaaatgatcttctttcaaac | | | |
| | a-3′-TAMRA | | | |
| Reverse | 5′-ttctgcttccactcttgacaa-3′ | 21 | 1659 | 365 |

TABLE ACB
Panel 5 Islet

| | Rel. Exp. (%) |
|---|---|
| | Ag7547, |
| | Run |
| Tissue Name | 308743747 |
| 97457_Patient-02go_adipose | 3.5 |
| 97476_Patient-07sk_skeletal muscle | 0.0 |
| 97477_Patient-07ut_uterus | 6.1 |
| 97478_Patient-07pl_placenta | 1.0 |
| 99167_Bayer Patient 1 | 4.8 |
| 97482_Patient-08ut_uterus | 3.7 |
| 97483_Patient-08pl_placenta | 1.4 |
| 97486_Patient-09sk_skeletal muscle | 7.3 |
| 97487_Patient-09ut_uterus | 4.6 |
| 97488_Patient-09pl_placenta | 0.8 |
| 97492_Patient-1Out_uterus | 8.0 |
| 97493_Patient-10pl_placenta | 3.3 |
| 97495_Patient-11go_adipose | 1.7 |
| 97496_Patient-11sk_skeletal muscle | 5.9 |
| 97497_Patient-11ut_uterus | 14.4 |
| 97498_Patient-11pl_placenta | 1.1 |
| 97500_Patient-12go_adipose | 3.7 |
| 9750l_Patient-12sk_skeletal muscle | 14.2 |
| 97502_Patient-12ut_uterus | 18.2 |
| 97503_Patient-12pl_placenta | 3.0 |
| 94721_Donor 2 U - A_Mesenchymal | 30.1 |
| Stem Cells | |
| 94722_Donor 2 U - B_Mesenchymal | 39.2 |
| Stem Cells | |
| 94723_Donor 2 U - C_Mesenchymal | 51.1 |
| Stem Cells | |
| 94709_Donor 2 AM - A_adipose | 23.3 |
| 9471O_Donor 2 AM - B_adipose | 23.0 |
| 94711_Donor 2 AM - C_adipose | 16.4 |
| 94712_Donor 2 AD - A_adipose | 43.5 |
| 94713_Donor 2 AD - B_adipose | 66.0 |
| 94714_Donor 2 AD - C_adipose | 47.0 |
| 94742_Donor 3 U - A_Mesenchymal | 21.9 |
| Stem Cells | |
| 94743_Donor 3 U - B_Mesenchymal | 27.7 |
| Stem Cells | |
| 94730_Donor 3 AM - A_adipose | 41.2 |
| 94731_Donor 3 AM - B_adipose | 43.5 |
| 94732_Donor 3 AM - C_adipose | 47.0 |
| 94733_Donor 3 AD - A adipose | 82.4 |
| 94734_Donor 3 AD - B adipose | 100.0 |
| 94735_Donor 3 AD - C_adipose | 31.9 |
| 77138_Liver_HepG2untreated | 4.5 |
| 73556_Heart_Cardiac | 0.5 |
| stromal cells (primary) | |
| 81735_Small Intestine | 4.8 |
| 72409_Kidney_Proximal Convoluted | 15.5 |
| Tubule | |
| 82685_Small intestine_Duodenum | 3.1 |
| 90650_Adrenal_{—} | 0.8 |
| Adrenocortical adenoma | |
| 72410_Kidney_HRCE | 49.0 |
| 72411_Kidney_HRE | 9.5 |
| 73139_Uterus_Uterine | 23.0 |
| smooth muscle cells | |

Panel 5 Islet Summary: Ag7547 Highest expression of this gene is detected in differentiated adipose tissue. Moderate levels of expression of this gene is mesenchymal stein cells, midway differentiated and differentiated adipose tissue. Low to moderate levels of expression of this gene is also detected in uterine smooth muscle, skeletal muscle from diabetic patient on insulin and kidney. Therefore, therapeutic modulation of this gene may be useful in the treatment of metabolic related diseases such as obesity, and diabetes.

AD. CG51264-03, and CG51264-04: (17941787-31) ST7-Like Protein.

Expression of gene CG51264-03 and CG51264-04 was assessed using the primer-probe sets Ag2725 and Ag2727, described in Tables ADA and ADB.

TABLE ADA

Probe Name Ag2725

| | | | | |
|---|---|---|---|---|
| | | | Start | |
| Primers | Sequences | Length | Position | SEQ ID No |

| | | | | |
|---|---|---|---|---|
| Forward | 5′-ctgcaactaccagaatcattgc- | 22 | 1415 | 366 |
| | 3′ | | | |
| Probe | TET-5′- | 26 | 1442 | 367 |
| | tggcaaacagaacccatctacttggt-3′-TAMRA | | | |
| Reverse | 5′-tgcaaggggatttaatgctact-3′ | 22 | 1469 | 368 |

TABLE ADB

Probe Name Ag2727

| | | | | |
|---|---|---|---|---|
| | | | Start | |
| Primers | Sequences | Length | Position | SEQ ID No |

| | | | | |
|---|---|---|---|---|
| Forward | 5′-ctgcaactaccagaatcattgc- | 22 | 1415 | 369 |
| | 3′ | | | |
| Probe | TET-5′- | 26 | 1442 | 370 |
| | tggcaaacagaacccatctacttggt | | | |
| | -3′-TAMRA | | | |
| Reverse | 5′-tgcaaggggatttaatgctact- | 22 | 1469 | 371 |
| | 3′ | | | |

AE. CG52423-01: PV1-Like Protein (3544179_EXT).

Expression of gene CG52423-01 was assessed using the primer-probe sets Ag1039, Ag1537, Ag760 and Ag4932, described in Tables AEA, AEB, AEC and AED. Results of the RTQ-PCR runs are shown in Tables AEE, AEF. AEG, AEH, AEI, AEJ, AEK, AEL, AEM and AEN.

TABLE AEA

Probe Name Ag1039

| | | | Start | |
|---|---|---|---|---|
| Primers | Sequences | Length | Position | SEQ ID No |
| Forward | 5′-aaggagcaactgcaaaaggt-3′ | 20 | 753 | 372 |
| Probe | TET-5′-ctgcccctggacaaggacaagttt-3′-TAMRA | 24 | 786 | 373 |
| Reverse | 5′-acaggttacgaaggtccatctc-3′ | 22 | 810 | 374 |

TABLE AEB

Probe Name Ag1537

| | | | | Start |
|---|---|---|---|---|
| Primers | Sequences | Length | Position | SEQ ID No |
| Forward | 5′-aaggagctggaagagaagaaga-3′ | 22 | 1197 | 375 |
| Probe | TET-5′-atcagaaactcagccctggacacctg-3′-TAMRA | 26 | 1251 | 376 |
| Reverse | 5′-gctgcgacttggtcttgat-3′ | 19 | 1278 | 377 |

TABLE AEC

Probe Name Ag760

| | | | Start | |
|---|---|---|---|---|
| Primers | Sequences | Length | Position | SEQ ID No |
| Forward | 5′-caccatgacaacgacacctata-3′ | 22 | 1924 | 378 |
| Probe | TET-5′-atatggcaccaacatcacatgcacg-3′-TAMRA | 25 | 1947 | 379 |
| Reverse | 5′-tgggtagaaagtgtgtgtgaaa-3′ | 22 | 1979 | 380 |

TABLE AED

Probe Name Ag4932

| | | | Start | |
|---|---|---|---|---|
| Primers | Sequences | Length | Position | SEQ ID No |
| Forward | 5′-aatgcagagatcaattcaagga-3′ | 22 | 535 | 381 |
| Probe | TET-5′-aacaagagctgcgatgccttgctctt-3′-TAMRA | 26 | 561 | 382 |
| Reverse | 5′-tcttcaccttctgattcagcat-3′ | 22 | 588 | 383 |

TABLE AEE
Ardais Panel v.1.0

| | Rel. Exp. (%) |
|---|---|
| | Ag1537, |
| | Run |
| Tissue Name | 267680189 |
| 136799_Lung cancer(362) | 23.8 |
| 136800_Lung NAT(363) | 15.6 |
| 136813_Lung cancer(372) | 45.4 |
| 136814_Lung NAT(373) | 14.4 |
| 136815_Lung cancer(374) | 39.2 |
| 136816_Lung NAT(375) | 100.0 |
| 136791_Lung cancer(35A) | 22.5 |
| 136795_Lung cancer(35E) | 35.4 |
| 136797_Lung cancer(360) | 22.4 |
| 136794_lung NAT(35D) | 14.3 |
| 136818_Lung NAT(377) | 33.0 |
| 136787_lung cancer(356) | 8.1 |
| 136788_lung NAT(357) | 52.5 |
| 136806_Lung cancer(36B) | 35.6 |
| 136807_Lung NAT(36C) | 18.8 |
| 136789_lung cancer(358) | 65.1 |
| 136802_Lung cancer(365) | 49.3 |
| 136803_Lung cancer(368) | 24.5 |
| 136804_Lung cancer(369) | 38.2 |
| 136811_Lung cancer(370) | 14.9 |
| 136810_Lung NAT(36F) | 31.4 |

TABLE AEF
CNS_neurodegeneration_v1.0

| | Rel. Exp. (%) | Rel. Exp. (%) |
|---|---|---|
| | Ag1537, | Ag4932, |
| | Run | Run |
| Tissue Name | 266937073 | 269217367 |
| AD 1 Hippo | 13.1 | 9.5 |
| AD 2 Hippo | 14.2 | 22.1 |
| AD 3 Hippo | 0.0 | 3.4 |
| AD 4 Hippo | 3.5 | 1.9 |
| AD 5 Hippo | 23.3 | 25.7 |
| AD 6 Hippo | 16.6 | 29.5 |
| Control 2 Hippo | 43.8 | 28.1 |
| Control 4 Hippo | 100.0 | 56.6 |
| Control (Path) 3 Hippo | 49.3 | 100.0 |
| AD 1 Temporal Ctx | 11.5 | 8.3 |
| AD 2 Temporal Ctx | 28.5 | 25.3 |
| AD 3 Temporal Ctx | 1.7 | 0.9 |
| AD 4 Temporal Ctx | 3.8 | 11.7 |
| AD 5 Inf Temporal Ctx | 31.0 | 36.3 |
| AD 5 Sup Temporal Ctx | 67.8 | 96.6 |
| AD 6 Inf Temporal Ctx | 23.7 | 38.2 |
| AD 6 Sup Temporal Ctx | 13.3 | 22.4 |
| Control 1 Temporal Ctx | 0.0 | 6.3 |
| Control 2 Temporal Ctx | 34.2 | 28.7 |
| Control 3 Temporal Ctx | 12.9 | 13.4 |
| Control 3 Temporal Ctx | 13.0 | 6.8 |
| Control (Path) 1 | 43.5 | 26.1 |
| Temporal Ctx | | |
| Control (Path) 2 | 12.2 | 10.0 |
| Temporal Ctx | | |
| Control (Path) 3 | 4.2 | 0.0 |
| Temporal Ctx | | |
| Control (Path) 4 | 1.5 | 5.7 |
| Temporal Ctx | | |
| AD 1 Occipital Ctx | 5.9 | 6.8 |
| AD 2 Occipital Ctx (Missing) | 0.0 | 0.0 |
| AD 3 Occipital Ctx | 0.0 | 1.6 |
| AD 4 Occipital Ctx | 5.4 | 4.2 |
| AD 5 Occipital Ctx | 25.2 | 18.8 |
| AD 6 Occipital Ctx | 4.3 | 6.9 |
| Control 1 Occipital Ctx | 0.0 | 0.0 |
| Control 2 Occipital Ctx | 19.3 | 14.0 |
| Control 3 Occipital Ctx | 23.5 | 8.2 |
| Control 4 Occipital Ctx | 3.3 | 4.1 |
| Control (Path) 1 | 15.4 | 13.5 |
| Occipital Ctx | | |
| Control (Path) 2 | 7.9 | 1.1 |
| Occipital Ctx | | |
| Control (Path) 3 | 0.0 | 1.0 |
| Occipital Ctx | | |
| Control (Path) 4 | 0.0 | 9.0 |
| Occipital Ctx | | |
| Control 1 Parietal Ctx | 3.4 | 0.8 |
| Control 2 Parietal Ctx | 23.7 | 22.2 |
| Control 3 Parietal Ctx | 4.0 | 0.0 |
| Control (Path) 1 | 28.3 | 14.0 |
| Parietal Ctx | | |
| Control (Path) 2 | 5.0 | 10.0 |
| Parietal Ctx | | |
| Control (Path) 3 | 0.0 | 1.2 |
| Parietal Ctx | | |
| Control (Path) 4 | 16.3 | 12.2 |
| Parietal Ctx | | |

TABLE AEG
General_screening_panel_v1.5

| | Rel. Exp. (%) |
|---|---|
| | Ag4932, |
| | Run |
| Tissue Name | 228843451 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 10.7 |
| Prostate ca.* (bone met) PC-3 | 0.0 |
| Prostate Pool | 14.7 |
| Placenta | 42.6 |
| Uterus Pool | 53.6 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.2 |
| Ovarian ca. IGROV-1 | 0.0 |
| Ovarian ca. OVCAR-8 | 0.1 |
| Ovary | 11.1 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 62.4 |
| Trachea | 47.3 |
| Lung | 4.3 |
| Fetal Lung | 17.2 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 1.2 |
| Fetal Liver | 44.1 |
| Liver ca. HepG2 | 0.0 |
| Kidney Pool | 55.1 |
| Fetal Kidney | 73.2 |
| Renal ca. 786-0 | 0.0 |
| Renal ca. A498 | 0.1 |
| Renal ca. ACHN | 0.0 |
| Renal ca. UO-31 | 0.0 |
| Renal ca. TK-10 | 0.0 |
| Bladder | 86.5 |
| Gastric ca. (liver met.) NCI-N87 | 4.5 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 51.1 |
| Colon ca. SW1116 | 0.0 |
| Colon ca. Colo-205 | 0.0 |
| Colon ca. SW-48 | 0.0 |
| Colon Pool | 89.5 |
| Small Intestine Pool | 11.7 |
| Stomach Pool | 37.9 |
| Bone Marrow Pool | 46.0 |
| Fetal Heart | 15.1 |
| Heart Pool | 22.2 |
| Lymph Node Pool | 66.9 |
| Fetal Skeletal Muscle | 23.2 |
| Skeletal Muscle Pool | 32.5 |
| Spleen Pool | 100.0 |
| Thymus Pool | 30.6 |
| CNS cancer (glio/astro) U87-MG | 0.0 |
| CNS cancer (glio/astro) U-118-MG | 0.1 |
| CNS cancer (neuro; met) SK-N-AS | 0.1 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.7 |
| Brain (Amygdala) Pool | 1.0 |
| Brain (cerebellum) | 2.0 |
| Brain (fetal) | 2.6 |
| Brain (Hippocampus) Pool | 2.1 |
| Cerebral Cortex Pool | 1.3 |
| Brain (Substantia nigra) Pool | 1.5 |
| Brain (Thalamus) Pool | 2.5 |
| Brain (whole) | 3.8 |
| Spinal Cord Pool | 1.9 |
| Adrenal Gland | 55.1 |
| Pituitary gland Pool | 10.3 |
| Salivary Gland | 20.7 |
| Thyroid (female) | 70.7 |
| Pancreatic ca. CAPAN2 | 0.0 |
| Pancreas Pool | 53.6 |

TABLE AEH
Oncology_cell_line_screening_panel_v3.2

| | Rel. Exp. (%) |
|---|---|
| | Ag1537, |
| | Run |
| Tissue Name | 267177741 |
| 94905_Daoy_Medulloblastoma/ | 0.0 |
| Cerebellum_sscDNA | |
| 94906_TE671_Medulloblastom/ | 0.0 |
| Cerebellum_sscDNA | |
| 94907_D283 | 0.0 |
| Med_Medulloblastoma/ | |
| Cerebellum_sscDNA | |
| 94908_PFSK-1_Primitive | 0.5 |
| Neuroectodermal/Cerebellum_sscDNA | |
| 94909_XF-498_CNS_sscDNA | 0.0 |
| 94910_SNB-78_CNS/glioma_sscDNA | 0.0 |
| 94911_SF- | 0.0 |
| 268_CNS/glioblastoma_sscDNA | |
| 94912_T98G_Glioblastoma_sscDNA | 0.0 |
| 96776_SK-N-SH_Neuroblastoma | 0.0 |
| (metastasis)_sscDNA | |
| 94913_SF- | 0.0 |
| 295_CNS/glioblastoma_sscDNA | |
| 132565_NT2 pool_sscDNA | 0.1 |
| 94914_Cerebellum_sscDNA | 0.2 |
| 96777_Cerebellum_sscDNA | 0.3 |
| 94916_NCI-H292_Mucoepidermoid lung | 0.0 |
| carcinoma_sscDNA | |
| 94917_DMS-114_Small cell lung | 0.0 |
| cancer_sscDNA | |
| 94918_DMS-79_Small cell lung | 0.0 |
| cancer/neuroendocrine_sscDNA | |
| 94919_NCI-H146_Small cell lung | 0.0 |
| cancer/neuroendocrine_sscDNA | |
| 94920_NCI-H526_Small cell lung | 0.0 |
| cancer/neuroendocrine_sscDNA | |
| 94921_NCI-N417_Small cell lung | 0.0 |
| cancer/neuroendocrine_sscDNA | |
| 94923_NCI-H82_Small cell lung | 0.0 |
| cancer/neuroendocrine_sscDNA | |
| 94924_NCI-H157_Squamous cell lung | 0.0 |
| cancer (metastasis)_sscDNA | |
| 94925_NCI-H1155_Large cell lung | 0.0 |
| cancer/neuroendocrine_sscDNA | |
| 94926_NCI-H1299_Large cell lung | 0.0 |
| cancer/neuroendocrine_sscDNA | |
| 94927_NCI-H727_Lung | 0.0 |
| carcinoid_sscDNA | |
| 94928_NCI-UMC-11_Lung | 0.0 |
| carcinoid_sscDNA | |
| 94929_LX-1_Small cell lung | 0.0 |
| cancer_sscDNA | |
| 94930_Colo-205_Colon cancer_sscDNA | 0.0 |
| 9493l_KM12_Colon cancer_sscDNA | 0.0 |
| 94932_KM20L2_Colon cancer_sscDNA | 0.0 |
| 94933_NCI-H716_Colon cancer_sscDNA | 0.0 |
| 94935_SW-48_Colon | 0.0 |
| adenocarcinoma_sscDNA | |
| 94936_SW1116_Colon | 0.0 |
| adenocarcinoma_sscDNA | |
| 94937_LS 174T_Colon | 0.0 |
| adenocarcinoma_sscDNA | |
| 94938_SW-948_Colon | 0.0 |
| adenocarcinoma_sscDNA | |
| 94939_SW-480_Colon | 0.0 |
| adenocarcinoma_sscDNA | |
| 94940_NCI-SNU-5_Gastric | 0.0 |
| carcinoma_sscDNA | |
| 112197_KATO III_Stomach_sscDNA | 0.0 |
| 94943_NCI-SNU-16_Gastric | 0.0 |
| carcinoma_sscDNA | |
| 94944_NCI-SNU-1_Gastric | 0.0 |
| carcinoma_sscDNA | |
| 94946_RF-1_Gastric | 0.0 |
| adenocarcinoma_sscDNA | |
| 94947_RF-48_Gastric | 0.1 |
| adenocarcinoma_sscDNA | |
| 96778_MKN-45_Gastric | 0.0 |
| carcinoma_sscDNA | |
| 94949_NCI-N87_Gastric | 0.0 |
| carcinoma_sscDNA | |
| 94951_OVCAR-5_Ovarian | 0.0 |
| carcinoma_sscDNA | |
| 94952_RL95-2_Uterine | 0.0 |
| carcinoma_sscDNA | |
| 94953_HelaS3_Cervical | 0.0 |
| adenocarcinoma_sscDNA | |
| 94954_Ca Ski_Cervical epidermoid | 0.2 |
| carcinoma (metastasis)_sscDNA | |
| 94955_ES-2_Ovarian clear cell | 0.0 |
| carcinoma_sscDNA | |
| 94957_Ramos/6 h stim_Stimulated | 0.0 |
| with PMA/ionomycin 6 h_sscDNA | |
| 94958_Ramos/14 h stim_Stimulated | 0.0 |
| with PMA/ionomycin 14 h_sscDNA | |
| 94962_MEG-01_Chronic | 0.7 |
| myelogenous leukemia | |
| (megokaryoblast)_sscDNA | |
| 94963_Raji_Burkitt's | 0.0 |
| lymphoma_sscDNA | |
| 94964_Daudi_Burkitt's | 0.8 |
| lymphoma_sscDNA | |
| 94965_U266_B-cell | 1.3 |
| plasmacytoma/myeloma_sscDNA | |
| 94968_CA46_Burkitt's | 0.2 |
| lymphoma_sscDNA | |
| 94970_RL_non-Hodgkin's B-cell | 0.0 |
| lymphoma_sscDNA | |
| 94972_JM1_pre-B-cell | 0.0 |
| lymphoma/leukemia_sscDNA | |
| 94973_Jurkat_T cell | 0.0 |
| leukemia_sscDNA | |
| 94974_TF-1_{—} | 100.0 |
| Erythroleukemia_sscDNA | |
| 94975_HUT 78_T-cell | 0.0 |
| lymphoma_sscDNA | |
| 94977_U937_Histiocytic | 0.0 |
| lymphoma_sscDNA | |
| 94980_KU-812_Myelogenous | 28.9 |
| leukemia_sscDNA | |
| 94981_769-P_Clear cell renal | 0.1 |
| carcinoma_sscDNA | |
| 94983_Caki-2_Clear cell renal | 0.0 |
| carcinoma_sscDNA | |
| 94984_SW 839_Clear cell renal | 0.0 |
| carcinoma_sscDNA | |
| 94986_G401_Wilms' tumor_sscDNA | 0.0 |
| 126768_293 cells_sscDNA | 0.0 |
| 94987_Hs766T_Pancreatic | 0.6 |
| carcinoma (LN metastasis)_sscDNA | |
| 94988_CAPAN-1_Pancreatic | 0.0 |
| adenocarcinoma (liver | |
| metastasis)_sscDNA | |
| 94989_SU86.86_Pancreatic | 0.0 |
| carcinoma (liver | |
| metastasis)_sscDNA | |
| 94990_BxPC-3_Pancreatic | 0.0 |
| adenocarcinoma_sscDNA | |
| 94991_HPAC_Pancreatic | 0.0 |
| adenocarcinoma_sscDNA | |
| 94992_MIA PaCa-2_Pancreatic | 0.0 |
| carcinoma_sscDNA | |
| 94993_CFPAC-1_Pancreatic ductal | 0.1 |
| adenocarcinoma_sscDNA | |
| 94994_PANC-1_Pancreatic | 0.0 |
| epithelioid ductal | |
| carcinoma_sscDNA | |
| 94996_T24_Bladder carcinma | 0.1 |
| (transitional cell)_sscDNA | |
| 94997_5637_Bladder | 0.0 |
| carcinoma_sscDNA | |
| 94998_HT-1197_Bladder | 0.0 |
| carcinoma_sscDNA | |
| 94999_UM-UC-3_Bladder carcinma | 0.0 |
| (transitional cell)_sscDNA | |
| 95000_A204_Rhabdomyosarcoma_{—} | 0.0 |
| sscDNA | |
| 95001_HT-1080_Fibrosarcoma_sscDNA | 0.0 |
| 95002_MG-63_Osteosarcoma | 0.0 |
| (bone)_sscDNA | |
| 95003_SK-LMS-1_{—} | 0.2 |
| Leiomyosarcoma | |
| (vulva)_sscDNA | |
| 95004_SJRH30_Rhabdomyosarcoma | 0.0 |
| (met to bone marrow)_sscDNA | |
| 95005_A431_Epidermoid | 0.0 |
| carcinoma_sscDNA | |
| 95007_WM266- | 0.0 |
| 4_Melanoma_sscDNA | |
| 112195_DU 145_Prostate_sscDNA | 0.0 |
| 95012_MDA-MB-468_Breast | 0.0 |
| adenocarcinoma_sscDNA | |
| 112196_SSC-4_Tongue_sscDNA | 0.0 |
| 112194_SSC-9_Tongue_sscDNA | 0.0 |
| 112191_SSC-15_Tongue_sscDNA | 0.0 |
| 95017_CAL 27_Squamous cell | 0.0 |
| carcinoma of tongue_sscDNA | |

TABLE AEI
Panel 1.2

| | Rel. Exp. (%) | Rel. Exp. (%) |
|---|---|---|
| | Ag1537, | Ag760, |
| | Run | Run |
| Tissue Name | 142331743 | 114246835 |
| Endothelial cells | 2.5 | 1.3 |
| Heart (Fetal) | 17.6 | 2.3 |
| Pancreas | 35.4 | 74.2 |
| Pancreatic ca. CAPAN 2 | 0.0 | 0.0 |
| Adrenal Gland | 37.4 | 19.1 |
| Thyroid | 14.9 | 100.0 |
| Salivary gland | 34.6 | 15.8 |
| Pituitary gland | 2.1 | 27.4 |
| Brain (fetal) | 0.1 | 0.7 |
| Brain (whole) | 0.2 | 0.5 |
| Brain (amygdala) | 0.3 | 0.3 |
| Brain (cerebellum) | 0.1 | 0.1 |
| Brain (hippocampus) | 0.8 | 0.7 |
| Brain (thalamus) | 0.6 | 0.4 |
| Cerebral Cortex | 0.8 | 0.3 |
| Spinal cord | 0.1 | 0.6 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 0.0 | 0.0 |
| astrocytoma SF-539 | 0.0 | 0.0 |
| astrocytoma SNB-75 | 0.0 | 0.0 |
| glioma SNB-19 | 0.0 | 0.0 |
| glioma U251 | 0.1 | 0.2 |
| glioma SF-295 | 0.1 | 0.1 |
| Heart | 50.3 | 17.0 |
| Skeletal Muscle | 18.2 | 16.0 |
| Bone marrow | 2.7 | 1.4 |
| Thymus | 0.9 | 2.8 |
| Spleen | 29.1 | 30.8 |
| Lymph node | 2.7 | 14.4 |
| Colorectal Tissue | 2.3 | 1.1 |
| Stomach | 11.5 | 33.2 |
| Small intestine | 52.5 | 41.5 |
| Colon ca. SW480 | 0.0 | 0.0 |
| Colon ca.* SW620 (SW480 met) | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon ca. Tissue (ODO3866) | 1.7 | 1.4 |
| Colon ca. HCC-2998 | 0.0 | 0.0 |
| Gastric ca.* (liver | 0.9 | 0.7 |
| met) NCI-N87 | | |
| Bladder | 52.5 | 13.1 |
| Trachea | 2.1 | 9.6 |
| Kidney | 100.0 | 22.4 |
| Kidney (fetal) | 23.8 | 31.9 |
| Renal ca. 786-0 | 0.0 | 0.0 |
| Renal ca. A498 | 0.1 | 0.1 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 2.1 | 1.6 |
| Liver (fetal) | 4.4 | 4.0 |
| Liver ca. | 0.0 | 0.1 |
| (hepatoblast) HepG2 | | |
| Lung | 1.0 | 4.1 |
| Lung (fetal) | 0.3 | 2.1 |
| Lung ca. (small cell) LX-1 | 0.0 | 0.0 |
| Lung ca. (small cell) | 0.0 | 0.0 |
| NCI-H69 | | |
| Lung ca. (s. cell var.) | 0.0 | 0.0 |
| SHP-77 | | |
| Lung ca. (large | 0.0 | 0.0 |
| cell)NCI-H460 | | |
| Lung ca. (non-sm. | 0.0 | 0.0 |
| cell) A549 | | |
| Lung ca. (non-s. cell) | 0.0 | 0.0 |
| NCI-H23 | | |
| Lung ca. (non-s. cell) | 0.0 | 0.0 |
| HOP-62 | | |
| Lung ca. (non-s. cl) | 0.0 | 0.0 |
| NCI-H522 | | |
| Lung ca. (squam.) SW 900 | 0.0 | 0.0 |
| Lung ca. (squam.) NCI-H596 | 0.0 | 0.0 |
| Mammary gland | 14.8 | 19.3 |
| Breast ca.* (pl. ef) MCF-7 | 0.0 | 0.0 |
| Breast ca.* (pl. ef) | 0.0 | 0.0 |
| MDA-MB-231 | | |
| Breast ca.* (pl. ef) T47D | 0.1 | 0.0 |
| Breast ca. BT-549 | 0.0 | 0.0 |
| Breast ca. MDA-N | 2.2 | 1.2 |
| Ovary | 3.0 | 0.8 |
| Ovarian ca. OVCAR-3 | 0.0 | 0.0 |
| Ovarian ca. OVCAR-4 | 0.0 | 0.0 |
| Ovarian ca. OVCAR-5 | 0.1 | 0.1 |
| Ovarian ca. OVCAR-8 | 0.2 | 0.1 |
| Ovarian ca. IGROV-1 | 0.0 | 0.0 |
| Ovarian ca. (ascites) | 0.0 | 0.0 |
| SK-OV-3 | | |
| Uterus | 9.2 | 12.8 |
| Placenta | 3.1 | 7.3 |
| Prostate | 19.5 | 12.3 |
| Prostate ca.* (bone | 0.0 | 0.0 |
| met) PC-3 | | |
| Testis | 0.2 | 1.4 |
| Melanoma Hs688(A).T | 0.0 | 0.0 |
| Melanoma* (met) | 0.0 | 0.0 |
| Hs688(B).T | | |
| Melanoma UACC-62 | 0.0 | 0.0 |
| Melanoma M14 | 0.0 | 0.0 |
| Melanoma LOX IMVI | 0.0 | 0.0 |
| Melanoma* (met) | 0.0 | 0.0 |
| SK-MEL-5 | | |

TABLE AEJ
Panel 1.3D

| | Rel. Exp. (%) |
|---|---|
| | Ag760, |
| | Run |
| Tissue Name | 165678100 |
| Liver adenocarcinoma | 0.0 |
| Pancreas | 43.8 |
| Pancreatic ca. CAPAN 2 | 0.0 |
| Adrenal gland | 21.5 |
| Thyroid | 79.6 |
| Salivary gland | 13.9 |
| Pituitary gland | 13.4 |
| Brain (fetal) | 0.7 |
| Brain (whole) | 0.9 |
| Brain (amygdala) | 1.6 |
| Brain (cerebellum) | 0.4 |
| Brain (hippocampus) | 1.8 |
| Brain (substantia nigra) | 2.3 |
| Brain (thalamus) | 2.7 |
| Cerebral Cortex | 0.7 |
| Spinal cord | 1.7 |
| glio/astro U87-MG | 0.0 |
| glio/astro U-118-MG | 0.1 |
| astrocytoma SW1783 | 0.0 |
| neuro*; met SK-N-AS | 0.0 |
| astrocytoma SF-539 | 0.1 |
| astrocytoma SNB-75 | 0.0 |
| glioma SNB-19 | 0.0 |
| glioma U251 | 0.7 |
| glioma SF-295 | 0.0 |
| Heart (fetal) | 6.9 |
| Heart | 11.O |
| Skeletal muscle (fetal) | 19.5 |
| Skeletal muscle | 9.9 |
| Bone marrow | 7.9 |
| Thymus | 6.9 |
| Spleen | 90.8 |
| Lymph node | 73.7 |
| Colorectal | 7.9 |
| Stomach | 65.5 |
| Small intestine | 100.0 |
| Colon ca. SW480 | 0.0 |
| Colon ca.* | 0.0 |
| SW620(SW480 met) | |
| Colon ca. HT29 | 0.0 |
| Colon ca. HCT-116 | 0.0 |
| Colon ca. CaCo-2 | 0.0 |
| Colon ca. | 24.0 |
| tissue(ODO3866) | |
| Colon ca. HCC-2998 | 0.0 |
| Gastric ca.* (liver met) | 1.7 |
| NCI-N87 | |
| Bladder | 17.1 |
| Trachea | 27.0 |
| Kidney | 18.2 |
| Kidney (fetal) | 33.4 |
| Renal ca. 786-0 | 0.0 |
| Renal ca. A498 | 0.2 |
| Renal ca. RXF 393 | 0.0 |
| Renal ca. ACHN | 0.0 |
| Renal ca. UO-31 | 0.0 |
| Renal ca. TK-10 | 0.0 |
| Liver | 1.9 |
| Liver (fetal) | 12.4 |
| Liver ca. (hepatoblast) | 0.0 |
| HepG2 | |
| Lung | 15.3 |
| Lung (fetal) | 6.1 |
| Lung ca. (small cell) LX-1 | 0.0 |
| Lung ca. (small cell) NCI-H69 | 0.0 |
| Lung ca. (s. cell var.) SHP-77 | 0.0 |
| Lung ca. (large cell)NCI-H460 | 0.4 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 0.0 |
| Mammary gland | 26.8 |
| Breast ca.* (pl. ef) MCF-7 | 0.0 |
| Breast ca.* (pl. ef) MDA- | 0.0 |
| MB-231 | |
| Breast ca.* (pl. ef) T47D | 0.0 |
| Breast ca. BT-549 | 0.0 |
| Breast ca. MDA-N | 0.2 |
| Ovary | 1.8 |
| Ovarian ca. OVCAR-3 | 0.1 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca.* (ascites) SK-OV-3 | 0.1 |
| Uterus | 87.7 |
| Placenta | 6.4 |
| Prostate | 11.3 |
| Prostate ca.* (bone met)PC-3 | 0.0 |
| Testis | 2.1 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 26.6 |

TABLE AEK
Panel 2D

| | Rel. Exp. (%) |
|---|---|
| | Ag1537, |
| | Run |
| Tissue Name | 145017308 |
| Normal Colon | 12.3 |
| CC Well to Mod Diff | 10.7 |
| (ODO3866) | |
| CC Margin (ODO3866) | 12.2 |
| CC Gr.2 rectosigmoid | 3.2 |
| (ODO3868) | |
| CC Margin (ODO3868) | 0.8 |
| CC Mod Diff (ODO3920) | 3.4 |
| CC Margin (ODO3920) | 2.2 |
| CC Gr.2 ascend colon | 13.4 |
| (ODO3921) | |
| CC Margin (ODO3921) | 5.8 |
| CC from Partial | 9.6 |
| Hepatectomy (ODO4309) | |
| Mets | |
| Liver Margin (ODO4309) | 0.6 |
| Colon mets to lung | 5.5 |
| (OD04451-01) | |
| Lung Margin (OD04451-02) | 0.8 |
| Normal Prostate 6546-1 | 14.1 |
| Prostate Cancer (OD04410) | 8.8 |
| Prostate Margin (OD04410) | 6.9 |
| Prostate Cancer (OD04720-01) | 3.1 |
| Prostate Margin (OD04720-02) | 10.3 |
| Normal Lung 061010 | 11.8 |
| Lung Met to Muscle | 6.4 |
| (ODO4286) | |
| Muscle Margin (ODO4286) | 9.9 |
| Lung Malignant Cancer | 19.3 |
| (OD03126) | |
| Lung Margin (OD03126) | 3.3 |
| Lung Cancer (OD04404) | 5.2 |
| Lung Margin (OD04404) | 25.3 |
| Lung Cancer (OD04565) | 3.4 |
| Lung Margin (OD04565) | 3.1 |
| Lung Cancer (OD04237-01) | 11.0 |
| Lung Margin (OD04237-02) | 18.2 |
| Ocular Mel Met to Liver | 0.7 |
| (ODO4310) | |
| Liver Margin (ODO4310) | 1.7 |
| Melanoma Mets to Lung | 3.9 |
| (OD04321) | |
| Lung Margin (OD04321) | 3.7 |
| Normal Kidney | 40.6 |
| Kidney Ca. Nuclear grade 2 | 5.7 |
| (OD04338) | |
| Kidney Margin (OD04338) | 11.1 |
| Kidney Ca Nuclear grade | 2.5 |
| 1/2 (OD04339) | |
| Kidney Margin (OD04339) | 17.6 |
| Kidney Ca. Clear cell type | 100.0 |
| (OD04340) | |
| Kidney Margin (OD04340) | 22.7 |
| Kidney Ca. Nuclear grade 3 | 55.1 |
| (OD04348) | |
| Kidney Margin (OD04348) | 19.9 |
| Kidney Cancer (OD04622-01) | 25.0 |
| Kidney Margin (OD04622-03) | 7.4 |
| Kidney Cancer (OD04450-01) | 1.3 |
| Kidney Margin (OD04450-03) | 9.2 |
| Kidney Cancer 8120607 | 9.2 |
| Kidney Margin 8120608 | 23.5 |
| Kidney Cancer 8120613 | 21.5 |
| Kidney Margin 8120614 | 12.3 |
| Kidney Cancer 9010320 | 34.4 |
| Kidney Margin 9010321 | 27.7 |
| Normal Uterus | 9.3 |
| Uterus Cancer 064011 | 6.4 |
| Normal Thyroid | 84.1 |
| Thyroid Cancer 064010 | 20.6 |
| Thyroid Cancer A302152 | 15.2 |
| Thyroid Margin A302153 | 21.3 |
| Normal Breast | 22.1 |
| Breast Cancer (OD04566) | 8.4 |
| Breast Cancer (OD04590-01) | 21.0 |
| Breast Cancer Mets | 27.7 |
| (OD04590-03) | |
| Breast Cancer Metastasis | 9.1 |
| (OD04655-05) | |
| Breast Cancer 064006 | 10.1 |
| Breast Cancer 1024 | 7.1 |
| Breast Cancer 9100266 | 10.4 |
| Breast Margin 9100265 | 7.4 |
| Breast Cancer A209073 | 27.4 |
| Breast Margin A209073 | 8.7 |
| Normal Liver | 1.1 |
| Liver Cancer 064003 | 6.5 |
| Liver Cancer 1025 | 0.7 |
| Liver Cancer 1026 | 8.1 |
| Liver Cancer 6004-T | 1.9 |
| Liver Tissue 6004-N | 3.6 |
| Liver Cancer 6005-T | 9.3 |
| Liver Tissue 6005-N | 0.6 |
| Normal Bladder | 14.1 |
| Bladder Cancer 1023 | 4.5 |
| Bladder Cancer A302173 | 3.6 |
| Bladder Cancer (OD04718-01) | 7.4 |
| Bladder Normal | 15.2 |
| Adjacent (OD04718-03) | |
| Normal Ovary | 1.4 |
| Ovarian Cancer 064008 | 6.5 |
| Ovarian Cancer (OD04768-07) | 1.6 |
| Ovary Margin (OD04768-08) | 9.2 |
| Normal Stomach | 13.5 |
| Gastric Cancer 9060358 | 2.8 |
| Stomach Margin 9060359 | 12.6 |
| Gastric Cancer 9060395 | 20.6 |
| Stomach Margin 9060394 | 7.5 |
| Gastric Cancer 9060397 | 10.0 |
| Stomach Margin 9060396 | 3.2 |
| Gastric Cancer 064005 | 6.7 |

TABLE AEL
Panel 4.1D

| | Rel. Exp. (%) |
|---|---|
| | Ag4932, |
| | Run |
| Tissue Name | 223597251 |
| Secondary Th1 act | 0.1 |
| Secondary Th2 act | 0.4 |
| Secondary Tr1 act | 0.1 |
| Secondary Th1 rest | 0.1 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 1.2 |
| CD45RO CD4 lymphocyte act | 0.2 |
| CD8 lymphocyte act | 0.1 |
| Secondary CD8 | 0.4 |
| lymphocyte rest | |
| Secondary CD8 | 0.0 |
| lymphocyte act | |
| CD4 lymphocyte none | 0.3 |
| 2ry Th1/Th2/Tr1_anti- | 0.0 |
| CD95 CH11 | |
| LAK cells rest | 0.1 |
| LAK cells IL-2 | 0.1 |
| LAK cells IL-2 + IL-12 | 0.2 |
| LAK cells IL-2 + IFN | 0.5 |
| gamma | |
| LAK cells IL-2 + IL-18 | 0.2 |
| LAK cells | 0.2 |
| PMA/ionomycin | |
| NK Cells IL-2 rest | 0.2 |
| Two Way MLR 3 day | 2.7 |
| Two Way MLR 5 day | 1.3 |
| Two Way MLR 7 day | 0.1 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.1 |
| B lymphocytes PWM | 0.0 |
| B lymphocytes CD40L | 0.5 |
| and IL-4 | |
| EOL-1 dbcAMP | 0.0 |
| EOL-1 dbcAMP | 0.1 |
| PMA/ionomycin | |
| Dendritic cells none | 0.1 |
| Dendritic cells LPS | 1.7 |
| Dendritic cells anti-CD40 | 0.9 |
| Monocytes rest | 0.6 |
| Monocytes LPS | 0.1 |
| Macrophages rest | 0.0 |
| Macrophages LPS | 0.1 |
| HUVEC none | 1.9 |
| HUVEC starved | 8.4 |
| HUVEC IL-1beta | 5.6 |
| HUVEC IFN gamma | 40.6 |
| HUVEC TNF alpha + IFN | 4.6 |
| gamma | |
| HUVEC TNF alpha + IL4 | 5.0 |
| HUVEC 1L-11 | 8.7 |
| Lung Microvascular EC none | 66.4 |
| Lung Microvascular EC | 30.4 |
| TNFalpha + IL-1beta | |
| Microvascular Dermal EC none | 43.5 |
| Microsvasular Dermal EC | 17.0 |
| TNFalpha + IL-1beta | |
| Bronchial epithelium | 0.3 |
| TNFalpha + IL1beta | |
| Small airway epithelium none | 0.0 |
| Small airway epithelium | 0.0 |
| TNFalpha + IL-1beta | |
| Coronery artery SMC rest | 0.0 |
| Coronery artery SMC | 1.1 |
| TNFalpha + IL-1beta | |
| Astrocytes rest | 0.0 |
| Astrocytes TNFalpha + IL- | 0.2 |
| 1beta | |
| KU-812 (Basophil) rest | 27.0 |
| KU-812 (Basophil) | 28.3 |
| PMA/ionomycin | |
| CCD1106 (Keratinocytes) none | 0.0 |
| CCD1106 (Keratinocytes) | 0.0 |
| TNFalpha + IL-1beta | |
| Liver cirrhosis | 20.6 |
| NCI-H292 none | 0.0 |
| NCI-H292 IL-4 | 0.0 |
| NCI-H292 IL-9 | 0.0 |
| NCI-H292 IL-13 | 0.1 |
| NCI-H292 IFN gamma | 0.0 |
| HPAEC none | 1.8 |
| HPAEC TNF alpha + IL-1 | 1.5 |
| beta | |
| Lung fibroblast none | 0.4 |
| Lung fibroblast TNF alpha + | 0.6 |
| IL-1 beta | |
| Lung fibroblast IL-4 | 0.2 |
| Lung fibroblast IL-9 | 0.0 |
| Lung fibroblast IL-13 | 0.4 |
| Lung fibroblast IFN gamma | 0.2 |
| Dermal fibroblast CCD1070 rest | 0.0 |
| Dermal fibroblast CCD1070 | 0.0 |
| TNF alpha | |
| Dermal fibroblast CCD1070 | 0.0 |
| IL-1 beta | |
| Dermal fibroblast IFN gamma | 1.1 |
| Dermal fibroblast IL-4 | 0.4 |
| Dermal Fibroblasts rest | 0.7 |
| Neutrophils TNFa + LPS | 0.4 |
| Neutrophils rest | 0.3 |
| Colon | 19.3 |
| Lung | 100.0 |
| Thymus | 48.0 |
| Kidney | 68.8 |

TABLE AEM
Panel 4D

| | Rel. Exp. (%) |
|---|---|
| | Ag760, |
| | Run |
| Tissue Name | 145803954 |
| Secondary Th1 act | 0.0 |
| Secondary Th2 act | 0.1 |
| Secondary Tr1 act | 0.0 |
| Secondary Th1 rest | 0.1 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.1 |
| Primary Th1 rest | 0.1 |
| Primary Th2 rest | 0.0 |
| Primary Tr1 rest | 0.0 |
| CD45RA CD4 lymphocyte act | 0.6 |
| CD45RO CD4 lymphocyte act | 0.2 |
| CD8 lymphocyte act | 0.0 |
| Secondary CD8 lymphocyte rest | 0.0 |
| Secondary CD8 lymphocyte act | 0.0 |
| CD4 lymphocyte none | 0.3 |
| 2ry Th1/Th2/Tr1_anti- | 0.0 |
| CD95 CH11 | |
| LAK cells rest | 0.1 |
| LAK cells IL-2 | 0.1 |
| LAK cells IL-2 + IL-12 | 0.0 |
| LAK cells IL-2 + IFN gamma | 1.0 |
| LAK cells IL-2 + IL-18 | 0.7 |
| LAK cells PMA/ionomycin | 0.0 |
| NK Cells IL-2 rest | 0.4 |
| Two Way MLR 3 day | 3.5 |
| Two Way MLR 5 day | 1.3 |
| Two Way MLR 7 day | 0.1 |
| PBMC rest | 0.1 |
| PBMC PWM | 0.0 |
| PBMC PHA-L | 0.1 |
| Ramos (B cell) none | 0.0 |
| Ramos (B cell) ionomycin | 0.1 |
| B lymphocytes PWM | 0.0 |
| B lymphocytes CD40L | 0.3 |
| and IL-4 | |
| EOL-1 dbcAMP | 0.0 |
| EOL-1 dbcAMP PMA/ionomycin | 0.0 |
| Dendritic cells none | 0.0 |
| Dendritic cells LPS | 2.3 |
| Dendritic cells anti-CD40 | 0.0 |
| Monocytes rest | 0.8 |
| Monocytes LPS | 0.0 |
| Macrophages rest | 0.0 |
| Macrophages LPS | 0.6 |
| HUVEC none | 3.8 |
| HUVEC starved | 16.8 |
| HUVEC IL-1beta | 3.4 |
| HUVEC IFN gamma | 36.6 |
| HUVEC TNF alpha + IFN | 4.0 |
| gamma | |
| HUVEC TNF alpha + IL4 | 3.4 |
| HUVEC IL-11 | 5.5 |
| Lung Microvascular EC none | 47.0 |
| Lung Microvascular EC | 22.8 |
| TNFalpha + IL-1beta | |
| Microvascular Dermal EC none | 40.1 |
| Microsvasular Dermal EC | 17.9 |
| TNFalpha + IL-1beta | |
| Bronchial epithelium | 0.0 |
| TNFalpha + IL1beta | |
| Small airway epithelium none | 0.0 |
| Small airway epithelium | 0.0 |
| TNFalpha + IL-1beta | |
| Coronery artery SMC rest | 0.0 |
| Coronery artery SMC | 0.0 |
| TNFalpha + IL-1beta | |
| Astrocytes rest | 0.0 |
| Astrocytes TNFalpha + IL- | 0.0 |
| 1beta | |
| KU-812 (Basophil) rest | 24.3 |
| KU-812 (Basophil) | 29.7 |
| PMA/ionomycin | |
| CCD1106 (Keratinocytes) none | 0.0 |
| CCD1106 (Keratinocytes) | 0.0 |
| TNFalpha + IL-1beta | |
| Liver cirrhosis | 19.5 |
| Lupus kidney | 34.4 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.9 |
| HPAEC TNF alpha + IL-1 | 0.7 |
| beta | |
| Lung fibroblast none | 0.0 |
| Lung fibroblast TNF alpha + | 0.0 |
| IL-1 beta | |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 | 0.0 |
| rest | |
| Dermal fibroblast CCD1070 | 0.0 |
| TNF alpha | |
| Dermal fibroblast CCD1070 | 0.1 |
| IL-1 beta | |
| Dermal fibroblast IFN gamma | 0.0 |
| Dermal fibroblast IL-4 | 0.1 |
| IBD Colitis 2 | 1.5 |
| IBD Crohn's | 9.0 |
| Colon | 40.3 |
| Lung | 100.0 |
| Thymus | 95.3 |
| Kidney | 59.9 |

TABLE AEN
general oncology screening panel_v_2.4

| | Rel.Exp. (%) | Rel.Exp. (%) |
|---|---|---|
| | Ag1537, Run | Ag760, Run |
| Tissue Name | 266930996 | 262228031 |
| Colon cancer 1 | 11.4 | 4.8 |
| Colon cancer NAT 1 | 9.4 | 1.7 |
| Colon cancer 2 | 4.1 | 3.9 |
| Colon cancer NAT 2 | 4.1 | 1.8 |
| Colon cancer 3 | 10.4 | 4.8 |
| Colon cancer NAT 3 | 7.6 | 1.2 |
| Colon malignant | 9.7 | 4.0 |
| cancer 4 | | |
| Colon normal | 3.4 | 2.3 |
| adjacent tissue 4 | | |
| Lung cancer 1 | 4.8 | 3.6 |
| Lung NAT 1 | 0.7 | 0.5 |
| Lung cancer 2 | 5.6 | 3.9 |
| Lung NAT 2 | 0.1 | 0.1 |
| Squamous cell | 5.4 | 2.4 |
| carcinoma 3 | | |
| Lung NAT 3 | 0.7 | 0.3 |
| metastatic | 1.5 | 1.1 |
| melanoma 1 | | |
| Melanoma 2 | 4.0 | 2.6 |
| Melanoma 3 | 3.8 | 1.2 |
| metastatic | 2.2 | 0.9 |
| melanoma 4 | | |
| metastatic | 4.6 | 1.5 |
| melanoma 5 | | |
| Bladder cancer 1 | 1.0 | 0.6 |
| Bladder cancer NAT 1 | 0.0 | 0.0 |
| Bladder cancer 2 | 4.4 | 2.1 |
| Bladder cancer NAT 2 | 0.6 | 0.4 |
| Bladder cancer NAT 3 | 0.8 | 0.2 |
| Bladder cancer NAT 4 | 4.2 | 2.3 |
| Prostate | 2.1 | 2.9 |
| adenocarcinoma 1 | | |
| Prostate | 1.2 | 0.5 |
| adenocarcinoma 2 | | |
| Prostate | 2.1 | 1.0 |
| adenocarcinoma 3 | | |
| Prostate | 6.0 | 3.3 |
| adenocarcinoma 4 | | |
| Prostate cancer NAT 5 | 3.0 | 1.1 |
| Prostate | 1.3 | 0.5 |
| adenocarcinoma 6 | | |
| Prostate | 1.2 | 0.7 |
| adenocarcinoma 7 | | |
| Prostate | 1.2 | 0.4 |
| adenocarcinoma 8 | | |
| Prostate | 4.8 | 2.7 |
| adenocarcinoma 9 | | |
| Prostate cancer NAT 10 | 0.6 | 0.5 |
| Kidney cancer 1 | 90.1 | 100.0 |
| Kidney NAT 1 | 5.0 | 3.5 |
| Kidney cancer 2 | 60.3 | 55.1 |
| Kidney NAT 2 | 9.8 | 6.0 |
| Kidney cancer 3 | 30.8 | 39.5 |
| Kidney NAT 3 | 5.4 | 1.2 |
| Kidney cancer 4 | 100.0 | 29.9 |
| Kidney NAT 4 | 6.7 | 1.6 |

Ardais Panel v.1.0 Summary: Ag1537 Highest expression of this gene is detected in normal lung sample (CT=26.7). In addition, high to moderate levels of expression is seen in both cancer and normal lung samples. Therefore, therapeutic modulation of the PV1 protein (PLVAP) encoded by this gene may be useful in the treatment of certain subtypes of lung cancer.

CNS_neurodegeneration_v1.0 Summary: Ag1537/Ag4932 Two experiments with different probe and primer sets are in good agreement. This panel confirms the expression of this gene at low levels in the brains of an independent group of individuals. However, no differential expression of this gene was detected between Alzheimer's diseased postmortem brains and those of non-demented controls in this experiment. See Panel 1.5 for a discussion of this gene in treatment of central nervous system disorders.

General_screening_panel_v1.5 Summary: Ag4932 Highest expression of this gene is detected in spleen (CT=26). In addition, high expression of this gene is also detected in tissues with metabolic/endocrine functions including pancreas, adipose, adrenal gland, thyroid, pituitary gland, skeletal muscle, heart, liver and the gastrointestinal tract. The PV-1-like protein is a plasma membrane protein with an extracellular domain. The extracellular domain of this protein makes it a potential antibody target for the treatment of endocrine/metabolically related diseases, such as obesity and diabetes.

Moderate levels of expression of this gene is also seen in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

In addition, this gene also shows high expression in colon cancer tissue, with moderate levels of expression in a gastric NCI-N87 cell line. Therefore, therapeutic modulation of this gene may be useful in the treatment of colon and gastric cancers.

HASS Panel v1.0 Summary: Ag1537 Expression of this gene is low/undetectable (CTs>34.9) across all of the samples on this panel (data not shown).

Oncology_cell_line_screening_panel_v3.2 Summary: Ag1537 Highest expressio of this gene is detected in TF-1 erythroleukemia cells (CT=28.6). Moderate levels of expression of this gene is restricted to erythroleukemia and myelogenous leukemia. Therefore, expression of this gene may be used to distinguish these leukemia samples from other samples in the panel and also, as marker to detect the presence of these leukemia. In addition, therapeutic modulation of this gene or its protein product may be useful in the treatment of erythroleukemia and myelogenous leukemia.

Panel 1.2 Summary: Ag760/Ag1537 Results from two experiments using different probe/primer sets are in reasonable agreement with highest expression of this gene in thyroid and kidney (CTs=20-21.6). Expression of this gene seems to be restricted to normal tissue and it is low or undectable in cancer cell lines. Thus, expression of this gene could be used to distinguish between normal tissues and cultured cancer cell lines.

In addition, expression of this gene is high (CT<27) in a wide range of metabolic tissues including pancreas, adrenal gland, thyroid, pituitary, adult and fetal heart, skeletal muscle and adult and fetal liver. Also, moderate levels of expression is seen in in all regions of the central nervous system examined, including amygdala, hippocampus, substantia nigra, thalamus, cerebellum, cerebral cortex, and spinal cord. This expression pattern is consistant to that seen in panel 1.5. See panel 1.5 for further discussion of this gene.

Panel 1.3D Summary: Ag760 Expression of this gene is highest in small intestine (CT=26). The expression pattern is similar to that observed in Panel 1.5 and 1.2. See panel 1.5 for and panel 1.2 for further discussion of this gene.

Panel 2D Summary: Ag1537 Expression of this gene is highest in a kidney cancer (OD04340) sample (CT=25). Overall, this gene is widely expressed across this panel with high to moderate expression in both normal and adjacent cancer tissue. However, this gene is more highly expressed in kidney cancer tissue than in adjacent normal tissue, consistent with expression pattern seen in panel 2.4. Therefore, this gene could be used to distinguish kidney cancers from normal kidney tissue. In addition, therapeutic modulation of this gene, through the use of small molecule drugs or antibodies, might be of benefit in the treatment of kidney cancer.

Panel 4.1D Summary: Ag4932 Highest expression of this gene is detected in lung (CT=28.5). In addition, moderate levels of expression of this gene is also seen in endothelial cells, basophils and normal tissues represented by colon, thymus and kidney. This gene codes for a variant of PV-1, a component of the endothelial fenestral and stomatal diaphragms. Expression of this gene is consistent with the pattern already reported for PV-1 (Stan et al., 1999, Proc. Natl. Acad. Sci. USA 96:13203-13207, PMID: 10557298; Stan et al., 2001, Genomics 72(3):304-13, PMID: 11401446). Antibodies raised against the PV-1 encoded by this gene could prevent transendothelial trafficking of inflammatory cells to different tissues sites and therefore have a potential use for treatment of inflammatory diseases including delayed type hypersensitivity, asthma, emphysema, rheumatoid arthritis and inflammatory bowel disease.

Moderate levels of expression of this gene is also seen in liver cirrhosis samples. Therefore, antibodies or small molecule therapeutics could reduce or inhibit fibrosis that occurs in liver cirrhosis.

Panel 4D Summary: Ag760 Expression of this gene is highest in lung and thymus (CTs=26.3). High expression of this gene is also seen in normal kidney and colon with more moderate expression in endothelial cells and basophils. Expression of this gene is consistent with the pattern seen in panel 4.1 D and also, with the published report (Stan et al., 1999, Proc. Natl. Acad. Sci. USA 96:13203-13207, PMID: 10557298; Stan et ai., 2001, Genomics 72(3):304-13, PMID: 11401446). See panel 4.1D for further discussion of this gene.

general oncology screening panel_v_{—}2.4 Summary: Ag1537/Ag760 Two experiments with different probe and primer sets are in excellent agreement. Highest expression of this gene is seen in a kidney cancer sample (CTs=22.6-25). Significant expression of this gene is seen in melanoma, colon, lung, prostate, bladder and kidney cancer as well as normal tissue samples. Expression of this gene is higher in kidney cancer as compared to corresponding normal control samples. Therefore, expression of this gene may be used to distinguish kidney cancer from normal tissue and also as a marker to detect kidney cancer. Furthermore, therapeutic modulation of this gene or its protein product through the use of antibodies or small molecule drug may be useful in the treatment of melanoma, kidney, colon, lung and prostate cancers.

AF. CG52919-01: SEZ-6-Like Protein (7520500).

Expression of gene CG52919-01 was assessed using the primer-probe set Ag2806, described in Table AFA. Results of the RTQ-PCR runs are shown in Tables AFB, AFC, AFD and AFE.

TABLE AFA

Probe Name Ag2806

Start

| | | | | |
|---|---|---|---|---|
| Primers | Sequences | Length | Position | SEQ ID No |

| | | | | |
|---|---|---|---|---|
| Forward | 5′-gatgatgaggagaccaccacta-3′ | 22 | 835 | 384 |
| Probe | TET-5′-atcatcaccaccaccatcaccacagt-3′-TAMRA | 26 | 865 | 385 |
| Reverse | 5′-caggtagctgacctggtgtct-3′ | 21 | 893 | 386 |

TABLE AFB
CNS_neurodegeneration_v1.0

| | Rel. Exp. (%) |
|---|---|
| | Ag2806, Run |
| Tissue Name | 206976054 |
| AD 1 Hippo | 10.4 |
| AD 2 Hippo | 15.1 |
| AD 3 Hippo | 4.1 |
| AD 4 Hippo | 4.6 |
| AD 5 hippo | 0.0 |
| AD 6 Hippo | 19.6 |
| Control 2 Hippo | 25.5 |
| Control 4 Hippo | 12.0 |
| Control (Path) 3 Hippo | 0.7 |
| AD 1 Temporal Ctx | 7.7 |
| AD 2 Temporal Ctx | 12.0 |
| AD 3 Temporal Ctx | 11.1 |
| AD 4 Temporal Ctx | 19.5 |
| AD 5 Inf Temporal Ctx | 87.7 |
| AD 5 Sup Temporal Ctx | 52.9 |
| AD 6 Inf Temporal Ctx | 16.4 |
| AD 6 Sup Temporal Ctx | 31.0 |
| Control 1 Temporal Ctx | 13.5 |
| Control 2 Temporal Ctx | 16.5 |
| Control 3 Temporal Ctx | 12.5 |
| Control 4 Temporal Ctx | 19.5 |
| Control (Path) 1 Temporal Ctx | 49.7 |
| Control (Path) 2 Temporal Ctx | 36.3 |
| Control (Path) 3 Temporal Ctx | 4.7 |
| Control (Path) 4 Temporal Ctx | 32.8 |
| AD 1 Occipital Ctx | 11.5 |
| AD 2 Occipital Ctx (Missing) | 0.0 |
| AD 3 Occipital Ctx | 5.5 |
| AD 4 Occipital Ctx | 18.9 |
| AD 5 Occipital Ctx | 12.1 |
| AD 6 Occipital Ctx | 31.6 |
| Control 1 Occipital Ctx | 2.9 |
| Control 2 Occipital Ctx | 57.8 |
| Control 3 Occipital Ctx | 13.5 |
| Control 4 Occipital Ctx | 4.0 |
| Control (Path) 1 Occipital Ctx | 100.0 |
| Control (Path) 2 Occipital Ctx | 13.8 |
| Control (Path) 3 Occipital Ctx | 0.9 |
| Control (Path) 4 Occipital Ctx | 14.8 |
| Control 1 Parietal Ctx | 13.1 |
| Control 2 Parietal Ctx | 45.4 |
| Control 3 Parietal Ctx | 9.6 |
| Control (Path) 1 Parietal Ctx | 53.2 |
| Control (Path) 2 Parietal Ctx | 22.7 |
| Control (Path) 3 Parietal Ctx | 0.6 |
| Control (Path) 4 Parietal Ctx | 31.4 |

TABLE AFC
Panel 1.3D

| | Rel. Exp. (%) |
|---|---|
| | Ag2806, Run |
| Tissue Name | 165519991 |
| Liver adenocarcinoma | 2.5 |
| Pancreas | 0.0 |
| Pancreatic ca. CAPAN 2 | 0.0 |
| Adrenal gland | 0.0 |
| Thyroid | 0.0 |
| Salivary gland | 1.5 |
| Pituitary gland | 6.0 |
| Brain (fetal) | 47.0 |
| Brain (whole) | 17.7 |
| Brain (amygdala) | 22.4 |
| Brain (cerebellum) | 100.0 |
| Brain (hippocampus) | 47.3 |
| Brain (substantia nigra) | 6.5 |
| Brain (thalamus) | 39.5 |
| Cerebral Cortex | 25.0 |
| Spinal cord | 5.9 |
| glio/astro U87-MG | 3.5 |
| glio/astro U-118-MG | 6.3 |
| astrocytoma SW1783 | 0.0 |
| neuro*; met SK-N-AS | 2.3 |
| astrocytoma SF-539 | 0.0 |
| astrocytoma SNB-75 | 4.1 |
| glioma SNB-19 | 1.1 |
| glioma U251 | 8.0 |
| glioma SF-295 | 2.0 |
| Heart (fetal) | 0.0 |
| Heart | 0.0 |
| Skeletal muscle (fetal) | 3.8 |
| Skeletal muscle | 0.0 |
| Bone marrow | 6.7 |
| Thymus | 3.7 |
| Spleen | 5.5 |
| Lymph node | 11.4 |
| Colorectal | 2.3 |
| Stomach | 2.3 |
| Small intestine | 8.7 |
| Colon ca. SW480 | 0.0 |
| Colon ca.* SW620(SW480 met) | 0.0 |
| Colon ca. HT29 | 1.8 |
| Colon ca. HCT-116 | 0.0 |
| Colon ca. CaCo-2 | 1.8 |
| Colon ca. tissue(ODO3866) | 2.2 |
| Colon ca. HCC-2998 | 5.1 |
| Gastric ca.* (liver met) NCI-N87 | 0.3 |
| Bladder | 5.0 |
| Trachea | 3.4 |
| Kidney | 0.0 |
| Kidney (fetal) | 4.8 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 2.3 |
| Lung ca. (small cell) LX-1 | 0.0 |
| Lung ca. (small cell) NCI-H69 | 11.8 |
| Lung ca. (s. cell var.) SHP-77 | 19.9 |
| Lung ca. (large cell)NCI-H460 | 0.0 |
| Lung ca. (non-sm. cell) A549 | 0.0 |
| Lung ca. (non-s. cell) NCI-H23 | 5.0 |
| Lung ca. (non-s. cell) HOP-62 | 0.0 |
| Lung ca. (non-s. cl) NCI-H522 | 3.4 |
| Lung ca. (squam.) SW 900 | 0.7 |
| Lung ca. (squam.) NCI-H596 | 84.7 |
| Mammary gland | 0.0 |
| Breast ca.* (pl. ef) MCF-7 | 1.5 |
| Breast ca.* (pl. ef) MDA-MB-231 | 1.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 3.8 |
| Ovarian ca. OVCAR-4 | 2.4 |
| Ovarian ca. OVCAR-5 | 0.0 |
| Ovarian ca. OVCAR-8 | 2.0 |
| Ovarian ca. IGROV-1 | 0.0 |
| Ovarian ca.* (ascites) SK-OV-3 | 0.0 |
| Uterus | 2.9 |
| Placenta | 3.6 |
| Prostate | 3.5 |
| Prostate ca.* (bone met)PC-3 | 0.0 |
| Testis | 3.8 |
| Melanoma Hs688(A).T | 0.0 |
| Melanoma* (met) Hs688(B).T | 0.0 |
| Melanoma UACC-62 | 0.0 |
| Melanoma M14 | 3.8 |
| Melanoma LOX IMVI | 0.0 |
| Melanoma* (met) SK-MEL-5 | 0.0 |
| Adipose | 3.2 |

TABLE AFD
Panel 2D

| | Rel. Exp. (%) |
|---|---|
| | Ag2806, Run |
| Tissue Name | 163577806 |
| Normal Colon | 1.2 |
| CC Well to Mod Diff (ODO3866) | 0.0 |
| CC Margin (ODO3866) | 0.0 |
| CC Gr.2 rectosigmoid (ODO3868) | 0.1 |
| CC Margin (ODO3868) | 0.1 |
| CC Mod Diff (ODO3920) | 0.3 |
| CC Margin (ODO3920) | 0.4 |
| CC Gr.2 ascend colon (ODO3921) | 0.5 |
| CC Margin (ODO3921) | 0.1 |
| CC from Partial | 0.3 |
| Hepatectomy (ODO4309) Mets | |
| Liver Margin (ODO4309) | 0.0 |
| Colon mets to lung (OD04451-01) | 0.2 |
| Lung Margin (OD04451-02) | 0.2 |
| Normal Prostate 6546-1 | 1.4 |
| Prostate Cancer (OD04410) | 0.5 |
| Prostate Margin (OD04410) | 0.9 |
| Prostate Cancer (OD04720-01) | 1.0 |
| Prostate Margin (OD04720-02) | 0.6 |
| Normal Lung 061010 | 0.9 |
| Lung Met to Muscle (ODO4286) | 0.0 |
| Muscle Margin (ODO4286) | 0.2 |
| Lung Malignant Cancer | 0.1 |
| (OD03126) | |
| Lung Margin (OD03126) | 0.3 |
| Lung Cancer (OD04404) | 0.1 |
| Lung Margin (OD04404) | 0.3 |
| Lung Cancer (OD04565) | 0.1 |
| Lung Margin (OD04565) | 0.3 |
| Lung Cancer (OD04237-01) | 0.2 |
| Lung Margin (OD04237-02) | 0.2 |
| Ocular Mel Met to Liver | 0.1 |
| (ODO4310) | |
| Liver Margin (ODO4310) | 0.0 |
| Melanoma Mets to Lung | 0.0 |
| (OD04321) | |
| Lung Margin (OD04321) | 0.4 |
| Normal Kidney | 0.3 |
| Kidney Ca, Nuclear grade 2 | 0.4 |
| (OD04338) | |
| Kidney Margin (OD04338) | 85.3 |
| Kidney Ca Nuclear grade | 0.2 |
| 1/2 (OD04339) | |
| Kidney Margin (OD04339) | 0.2 |
| Kidney Ca, Clear cell type | 0.1 |
| (OD04340) | |
| Kidney Margin (OD04340) | 0.2 |
| Kidney Ca, Nuclear grade 3 | 0.0 |
| (OD04348) | |
| Kidney Margin (OD04348) | 0.3 |
| Kidney Cancer (OD04622-01) | 0.1 |
| Kidney Margin (OD04622-03) | 0.0 |
| Kidney Cancer (OD04450-01) | 0.2 |
| Kidney Margin (OD04450-03) | 0.2 |
| Kidney Cancer 8120607 | 0.0 |
| Kidney Margin 8120608 | 0.2 |
| Kidney Cancer 8120613 | 0.0 |
| Kidney Margin 8120614 | 0.3 |
| Kidney Cancer 9010320 | 0.4 |
| Kidney Margin 9010321 | 0.2 |
| Normal Uterus | 0.4 |
| Uterus Cancer 064011 | 0.7 |
| Normal Thyroid | 0.1 |
| Thyroid Cancer 064010 | 0.0 |
| Thyroid Cancer A302152 | 0.2 |
| Thyroid Margin A302153 | 0.1 |
| Normal Breast | 0.4 |
| Breast Cancer (OD04566) | 100.0 |
| Breast Cancer (OD04590-01) | 0.2 |
| Breast Cancer Mets | 0.3 |
| (OD04590-03) | |
| Breast Cancer Metastasis | 0.1 |
| (OD04655-05) | |
| Breast Cancer 064006 | 0.1 |
| Breast Cancer 1024 | 0.7 |
| Breast Cancer 9100266 | 0.1 |
| Breast Margin 9100265 | 0.1 |
| Breast Cancer A209073 | 0.3 |
| Breast Margin A209073 | 0.3 |
| Normal Liver | 0.1 |
| Liver Cancer 064003 | 0.0 |
| Liver Cancer 1025 | 0.1 |
| Liver Cancer 1026 | 0.3 |
| Liver Cancer 6004-T | 0.1 |
| Liver Tissue 6004-N | 0.2 |
| Liver Cancer 6005-T | 0.2 |
| Liver Tissue 6005-N | 0.0 |
| Normal Bladder | 0.2 |
| Bladder Cancer 1023 | 0.2 |
| Bladder Cancer A302173 | 0.2 |
| Bladder Cancer (OD04718-01) | 0.1 |
| Bladder Normal | 0.5 |
| Adjacent (OD04718-03) | |
| Normal Ovary | 0.1 |
| Ovarian Cancer 064008 | 0.2 |
| Ovarian Cancer (OD04768-07) | 0.1 |
| Ovary Margin (OD04768-08) | 0.1 |
| Normal Stomach | 0.8 |
| Gastric Cancer 9060358 | 0.1 |
| Stomach Margin 9060359 | 0.1 |
| Gastric Cancer 9060395 | 0.3 |
| Stomach Margin 9060394 | 0.5 |
| Gastric Cancer 9060397 | 0.1 |
| Stomach Margin 9060396 | 0.1 |
| Gastric Cancer 064005 | 0.2 |

TABLE AFE
Panel 4D

| | Rel. Exp. (%) |
|---|---|
| | Ag2806, Run |
| Tissue Name | 162330998 |
| Secondary Th1 act | 20.3 |
| Secondary Th2 act | 5.1 |
| Secondary Tr1 act | 9.7 |
| Secondary Th1 rest | 21.9 |
| Secondary Th2 rest | 34.6 |
| Secondary Tr1 rest | 0.0 |
| Primary Th1 act | 11.2 |
| Primary Th2 act | 13.2 |
| Primary Tr1 act | 0.0 |
| Primary Th1 rest | 37.6 |
| Primary Th2 rest | 10.0 |
| Primary Tr1 rest | 2.3 |
| CD45RA CD4 lymphocyte act | 11.0 |
| CD45RO CD4 lymphocyte act | 11.8 |
| CD8 lymphocyte act | 46.7 |
| Secondary CD8 lymphocyte rest | 44.4 |
| Secondary CD8 lymphocyte act | 10.2 |
| CD4 lymphocyte none | 43.2 |
| 2ry Th1/Th2/Tr1_anti-CD95 CH11 | 54.7 |
| LAK cells rest | 13.2 |
| LAK cells IL-2 | 17.1 |
| LAK cells IL-2 + IL-12 | 17.4 |
| LAK cells IL-2 + IFN gamma | 70.2 |
| LAK cells IL-2 + IL-18 | 2.5 |
| LAK cells PMA/ionomycin | 5.0 |
| NK Cells IL-2 rest | 21.0 |
| Two Way MLR 3 day | 43.5 |
| Two Way MLR 5 day | 18.0 |
| Two Way MLR 7 day | 0.0 |
| PBMC rest | 7.2 |
| PBMC PWM | 48.3 |
| PBMC PHA-L | 76.8 |
| Ramos (B cell) none | 0.0 |
| Ramos (B cell) ionomycin | 0.0 |
| B lymphocytes PWM | 28.7 |
| B lymphocytes CD40L and IL-4 | 100.0 |
| EOL-1 dbcAMP | 21.2 |
| EOL-1 dbcAMP PMA/ionomycin | 50.7 |
| Dendritic cells none | 15.3 |
| Dendritic cells LPS | 32.3 |
| Dendritic cells anti-CD40 | 31.4 |
| Monocytes rest | 52.5 |
| Monocytes LPS | 42.3 |
| Macrophages rest | 11.0 |
| Macrophages LPS | 0.0 |
| HUVEC none | 0.0 |
| HUVEC starved | 11.7 |
| HUVEC IL-1beta | 0.0 |
| HUVEC IFN gamma | 21.9 |
| HUVEC TNF alpha + IFN gamma | 0.0 |
| HUVEC TNF alpha + IL4 | 0.0 |
| HUVEC IL-11 | 5.6 |
| Lung Microvascular EC none | 0.0 |
| Lung Microvascular EC | 0.0 |
| TNFalpha + IL-1beta | |
| Microvascular Dermal EC none | 0.0 |
| Microsvasular Dermal EC | 12.8 |
| TNFalpha + IL-1beta | |
| Bronchial epithelium | 12.1 |
| TNFalpha + IL1beta | |
| Small airway epithelium none | 0.0 |
| Small airway epithelium | 0.0 |
| TNFalpha + IL-1beta | |
| Coronery artery SMC rest | 0.0 |
| Coronery artery SMC | 4.4 |
| TNFalpha + IL-1beta | |
| Astrocytes rest | 12.8 |
| Astrocytes TNFalpha + IL-1beta | 11.3 |
| KU-812 (Basophil) rest | 0.0 |
| KU-812 (Basophil) | 0.0 |
| PMA/ionomycin | |
| CCD1106 (Keratinocytes) none | 0.0 |
| CCD1106 (Keratinocytes) | 0.0 |
| TNFalpha + IL-1beta | |
| Liver cirrhosis | 96.6 |
| Lupus kidney | 0.0 |
| NCI-H292 none | 22.8 |
| NCI-H292 IL-4 | 0.0 |
| NCI-H292 IL-9 | 23.2 |
| NCI-H292 IL-13 | 32.1 |
| NCI-H292 IFN gamma | 11.4 |
| HPAEC none | 0.0 |
| HPAEC TNF alpha + IL-1 beta | 0.0 |
| Lung fibroblast none | 0.0 |
| Lung fibroblast TNF alpha + | 0.0 |
| IL-1 beta | |
| Lung fibroblast IL-4 | 0.0 |
| Lung fibroblast IL-9 | 12.2 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 | 85.3 |
| TNF alpha | |
| Dermal fibroblast CCD1070 | 0.0 |
| IL-1 beta | |
| Dermal fibroblast IFN gamma | 0.0 |
| Dermal fibroblast IL-4 | 0.0 |
| IBD Colitis 2 | 12.1 |
| IBD Crohn's | 5.6 |
| Colon | 94.0 |
| Lung | 27.7 |
| Thymus | 34.6 |
| Kidney | 78.5 |

CNS_neurodegeneration_v1.0 Summary: Ag2806 This panel confirms the expression of this gene at low levels in the brains of an independent group of individuals. However, no differential expression of this gene was detected between Alzheimer's diseased postmortem brains and those of non-demented controls in this experiment. See Panel 1.3D for a discussion of this gene in treatment of central nervous system disorders.

Panel 1.3D Summary: Ag2806 Highest expression of this gene is detected in brain cerebellum (CT=31.2). Moderate levels of expression of this gene is mainly seen in all the regions of brain including amygdala, hippocampus, substantia nigra, thalamus, cerebellum, cerebral cortex, and spinal cord. Therefore, therapeutic modulation of this gene product may be useful in the treatment of central nervous system disorders such as Alzheiiner's disease, Parkinson's disease, epilepsy, multiple sclerosis, schizophrenia and depression.

This gene codes for a homolog of mouse seizure related protein, SEZ-6. Mouse SEZ-6 was first isolated from cerebrum cortex-derived cells treated with pentylentetrazole (PTZ), one of the convulsant drugs (Shimizu-Nishikawa et al., 1995, Brain Res Mol Brain Res 28(2):201-10, PMID: 7723619). Thus, SEZ-6 protein encoded by this gene may also play a role in brain seizure.

In addition, moderate to low levels of expression of this gene is also seen in three lung cancer cell lines. Therefore, expression of this gene may be used as diagnostic marker to detect lung cancer and also, modulation of this gene or its protein product through the use of antibody or protein therapeutics, may be useful in the treatment of lung cancer.

Panel 2D Summary: Ag2806 Highest expression of this gene is detected in breast cancer and normal kidney (CTs=26). Low levels of expression of this gene is also seen in breast, prostate, colon, uterine and kidney cancer. Therefore, therapeutic modulation of this gene product through the use of antibodies may be useful in the treatment of these cancers.

Panel 4D Summary: Ag2806 Highest expression of this gene is detected in CD40L and IL-4 treated B lymphocytes (CT=34). Low but significant levels of expression of this gene is also seen in TNF alpha treated dermal fibroblasts, IL-2+IFN gamma treated LAK cells, PHA-L treated PBMC cells, liver cirrhosis and normal tissue represented by colon and kidney. Therefore, therapeutic modulation of this gene may be useful in the treatment of autoimmune and inflammatory diseases such as lupus erythematosus, Crohn's disease, ulcerative colitis, multiple sclerosis, chronic obstructive pulmonary disease, asthma, emphysema, rheumatoid arthritis, or psoriasis and liver cirrhosis.

AG., CG52919-02, CG52919-03 and CG52919-04: SEZ6-Like Protein (7520500-54-1).

Expression of gene CG52919-02, CG52919-03 and CG52919-04 was assessed using the primer-probe sets Ag2795, Ag2807, Ag90 and Ag7017, described in Tables AGA, AGB, AGC and AGD. Results of the RTQ-PCR runs are shown in Tables AGE, AGF, AGG, AGH, AGI, AGJ, AGK, AGL, AGM and AGN.

TABLE AGA

Probe Name Ag2795

| | | | | |
|---|---|---|---|---|
| | | | Start | |
| Primers | Sequences | Length | Position | SEQ ID No |

| | | | | |
|---|---|---|---|---|
| Forward | 5′-cctacaaccgcattaccataga-3′ | 22 | 1670 | 387 |
| Probe | TET-5′-tcagcgtttgacaatccaacttacga-3′-TAMRA | 26 | 1693 | 388 |
| Reverse | 5′-cccacctagatggagacttcat-3′ | 22 | 1739 | 389 |

TABLE AGB

Probe Name Ag2807

| | | | | |
|---|---|---|---|---|
| | | | Start | |
| Primers | Sequences | Length | Position | SEQ ID No |

| | | | | |
|---|---|---|---|---|
| Forward | 5′-cctacaaccgcattaccataga-3′ | 22 | 1670 | 390 |
| Probe | TET-5′-tcagcgtttgacaatccaacttacga-3′-TAMRA | 26 | 1693 | 391 |
| Reverse | 5′-ccaacctagatggagacttcat-3′ | 22 | 1739 | 392 |

TABLE AGC

Probe Name Ag90

| | | | Start | |
|---|---|---|---|---|
| Primers | Sequences | Length | Position | SEQ ID No |
| Forward | 5′-ttggcctggactgcttccttc-3′ | 20 | 977 | 393 |
| Probe | TET-5′-catctctgtctaccctggctatggcgtg-3′-TAMRA | 28 | 999 | 394 |
| Reverse | 5′-aggctgatattctggaccttgatt-3′ | 24 | 1029 | 395 |

TABLE AGD

Probe Name Ag7017

| | | | Start | |
|---|---|---|---|---|
| Primers | Sequences | Length | Position | SEQ ID No |
| Forward | 5′-gtttgacaatccaacttacgagac-3′ | 24 | 1698 | 396 |
| Probe | TET-5′-cctagatggagacttcatattctctcgtct-3′-TAMRA | 30 | 1727 | 397 |
| Reverse | 5′-caagtctgagttgacttccctagac-3′ | 25 | 1765 | 398 |

TABLE AGE
AI_comprehensive panel_v1.0

| | Rel. Exp. (%) |
|---|---|
| | Ag2795, Run |
| Tissue Name | 255324382 |
| 110967 COPD-F | 0.0 |
| 110980 COPD-F | 0.0 |
| 110968 COPD-M | 0.0 |
| 110977 COPD-M | 0.1 |
| 110989 Emphysema-F | 0.0 |
| 110992 Emphysema-F | 0.0 |
| 110993 Emphysema-F | 0.0 |
| 110994 Emphysema-F | 0.0 |
| 110995 Emphysema-F | 0.2 |
| 110996 Emphysema-F | 0.0 |
| 110997 Asthma-M | 0.0 |
| 111001 Asthma-F | 0.1 |
| 111002 Asthma-F | 0.0 |
| 111003 Atopic Asthma-F | 0.0 |
| 111004 Atopic Asthma-F | 0.0 |
| 111005 Atopic Asthma-F | 0.0 |
| 111006 Atopic Asthma-F | 0.0 |
| 111417 Allergy-M | 0.0 |
| 112347 Allergy-M | 0.0 |
| 112349 Normal Lung-F | 0.0 |
| 112357 Normal Lung-F | 0.1 |
| 112354 Normal Lung-M | 0.0 |
| 112374 Crohns-F | 0.0 |
| 112389 Match Control Crohns-F | 0.0 |
| 112375 Crohns-F | 0.2 |
| 112732 Match Control Crohns-F | 0.0 |
| 112725 Crohns-M | 0.0 |
| 112387 Match Control Crohns-M | 0.0 |
| 112378 Crohns-M | 0.0 |
| 112390 Match Control Crohns-M | 0.2 |
| 112726 Crohns-M | 0.1 |
| 112731 Match Control Crohns-M | 0.1 |
| 112380 Ulcer Col-F | 0.1 |
| 112734 Match Control | 0.1 |
| Ulcer Col-F | |
| 112384 Ulcer Col-F | 0.4 |
| 112737 Match Control | 0.0 |
| Ulcer Col-F | |
| 112386 Ulcer Col-F | 0.0 |
| 112738 Match Control | 0.0 |
| Ulcer Col-F | |
| 112381 Ulcer Col-M | 0.0 |
| 112735 Match Control | 0.7 |
| Ulcer Col-M | |
| 112382 Ulcer Col-M | 0.0 |
| 112394 Match Control | 0.0 |
| Ulcer Col-M | |
| 112383 Ulcer Col-M | 0.1 |
| 112736 Match Control | 0.0 |
| Ulcer Col-M | |
| 112423 Psoriasis-F | 0.1 |
| 112427 Match Control | 0, |
| Psoriasis-F | |
| 112418 Psoriasis-M | 0.0 |
| 112723 Match Control | 0.0 |
| Psoriasis-M | |
| 112419 Psoriasis-M | 0.0 |
| 112424 Match Control | 0.1 |
| Psoriasis-M | |
| 112420 Psoriasis-M | 0.1 |
| 112425 Match Control | 0.1 |
| Psoriasis-M | |
| 104689 (MF) OA Bone-Backus | 0.1 |
| 104690 (MF) Adj | 0.3 |
| “Normal” Bone-Backus | |
| 104691 (MF) OA Synovium-Backus | 0.0 |
| 104692 (BA) OA Cartilage-Backus | 0.0 |
| 104694 (BA) OA Bone-Backus | 0.1 |
| 104695 (BA) Adj | 0.0 |
| “Normal” Bone-Backus | |
| 104696 (BA) OA Synovium-Backus | 0.0 |
| 104700 (SS) OA Bone-Backus | 0.0 |
| 104701 (SS) Adj | 0.0 |
| “Normal” Bone-Backus | |
| 104702 (SS) OA Synovium-Backus | 0.0 |
| 117093 OA Cartilage Rep7 | 0.0 |
| 112672 OA Bone5 | 0.0 |
| 112673 OA Synovium5 | 0.0 |
| 112674 OA Synovial | 0.0 |
| Fluid cells 5 | |
| 117100 OA Cartilage Rep14 | 0.0 |
| 112756 OA Bone9 | 100.0 |
| 112757 OA Synovium9 | 0.0 |
| 112758 OA Synovial | 0.0 |
| Fluid Cells9 | |
| 117125 RA Cartilage Rep2 | 0.1 |
| 113492 Bone2 RA | 0.0 |
| 113493 Synovium2 RA | 0.0 |
| 113494 Syn Fluid Cells RA | 0.0 |
| 113499 Cartilage4 RA | 0.0 |
| 113500 Bone4 RA | 0.2 |
| 113501 Synovium4 RA | 0.0 |
| 113502 Syn Fluid Cells4 RA | 0.0 |
| 113495 Cartilage3 RA | 0.0 |
| 113496 Bone3 RA | 0.3 |
| 113497 Synovium3 RA | 0.2 |
| 113498 Syn Fluid Cells3 RA | 0.0 |
| 117106 Normal Cartilage Rep20 | 0.0 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.0 |
| 113667 Bone4 Normal | 0.0 |
| 113668 Synovium4 Normal | 0.0 |
| 113669 Syn Fluid Cells4 Normal | 0.0 |

TABLE AGF
CNS_neurodegeneration_v1.0

| | Rel. | Rel. | Rel. |
|---|---|---|---|
| | Exp. (%) | Exp. (%) | Exp. (%) |
| | Ag2795, | Ag2807, | Ag7017, |
| | Run | Run | Run |
| Tissue Name | 206976052 | 206482282 | 279032451 |
| AD 1 Hippo | 13.4 | 14.6 | 10.6 |
| AD 2 Hippo | 66.4 | 80.1 | 62.4 |
| AD 3 Hippo | 7.5 | 6.0 | 7.3 |
| AD 4 Hippo | 7.4 | 11.1 | 9.5 |
| AD 5 hippo | 0.0 | 77.4 | 61.6 |
| AD 6 Hippo | 53.6 | 49.7 | 46.0 |
| Control 2 | 49.7 | 48.3 | 51.8 |
| Hippo | | | |
| Control 4 | 6.6 | 8.1 | 7.3 |
| Hippo | | | |
| Control | 2.0 | 2.4 | 0.0 |
| (Path) 3 | | | |
| Hippo | | | |
| AD 1 | 6.8 | 6.9 | 9.3 |
| Temporal | | | |
| Ctx | | | |
| AD 2 | 27.9 | 34.4 | 25.7 |
| Temporal | | | |
| Ctx | | | |
| AD 3 | 4.1 | 2.3 | 6.5 |
| Temporal | | | |
| Ctx | | | |
| AD 4 | 19.1 | 20.7 | 21.2 |
| Temporal | | | |
| Ctx | | | |
| AD 5 Inf | 59.5 | 69.3 | 100.0 |
| Temporal | | | |
| Ctx | | | |
| AD 5 | 46.0 | 45.7 | 39.5 |
| SupTemporal | | | |
| Ctx | | | |
| AD 6 Inf | 18.9 | 26.2 | 21.5 |
| Temporal | | | |
| Ctx | | | |
| AD 6 Sup | 20.9 | 21.2 | 22.2 |
| Temporal | | | |
| Ctx | | | |
| Control 1 | 3.1 | 2.5 | 3.9 |
| Temporal | | | |
| Ctx | | | |
| Control 2 | 48.3 | 56.6 | 52.5 |
| Temporal | | | |
| Ctx | | | |
| Control 3 | 12.6 | 15.4 | 14.7 |
| Temporal | | | |
| Ctx | | | |
| Control 4 | 5.9 | 8.5 | 6.7 |
| Temporal | | | |
| Ctx | | | |
| Control | 74.7 | 81.2 | 71.2 |
| (Path) 1 | | | |
| Temporal | | | |
| Ctx | | | |
| Control | 29.1 | 40.1 | 30.4 |
| (Path) 2 | | | |
| Temporal | | | |
| Ctx | | | |
| Control | 3.5 | 2.4 | 3.9 |
| (Path) 3 | | | |
| Temporal | | | |
| Ctx | | | |
| Control | 25.2 | 28.1 | 23.3 |
| (Path) 4 | | | |
| Temporal | | | |
| Ctx | | | |
| AD 1 | 6.7 | 7.9 | 9.3 |
| Occipital Ctx | | | |
| AD 2 | 0.0 | 0.0 | 0.0 |
| Occipital Ctx | | | |
| (Missing) | | | |
| AD 3 | 1.8 | 1.7 | 2.6 |
| Occipital Ctx | | | |
| AD 4 | 19.9 | 20.9 | 21.0 |
| Occipital Ctx | | | |
| AD 5 | 7.9 | 9.8 | 5.6 |
| Occipital Ctx | | | |
| AD 6 | 54.3 | 66.4 | 50.3 |
| Occipital Ctx | | | |
| Control 1 | 1.3 | 2.2 | 1.8 |
| Occipital Ctx | | | |
| Control 2 | 60.7 | 69.3 | 97.3 |
| Occipital Ctx | | | |
| Control 3 | 14.2 | 16.3 | 13.4 |
| Occipital Ctx | | | |
| Control 4 | 3.4 | 2.9 | 3.4 |
| Occipital Ctx | | | |
| Control | 100.0 | 100.0 | 79.6 |
| (Path) 1 | | | |
| Occipital Ctx | | | |
| Control | 8.5 | 7.6 | 6.6 |
| (Path) 2 | | | |
| Occipital Ctx | | | |
| Control | 0.7 | 1.2 | 1.7 |
| (Path) 3 | | | |
| Occipital Ctx | | | |
| Control | 10.0 | 11.0 | 10.6 |
| (Path) 4 | | | |
| Occipital Ctx | | | |
| Control 1 | 6.4 | 5.0 | 3.5 |
| Parietal Ctx | | | |
| Control 2 | 22.8 | 26.2 | 24.7 |
| Parietal Ctx | | | |
| Control 3 | 14.9 | 20.0 | 20.4 |
| Parietal Ctx | | | |
| Control | 74.2 | 92.7 | 84.7 |
| (Path) 1 | | | |
| Parietal Ctx | | | |
| Control | 20.3 | 20.4 | 20.0 |
| (Path) 2 | | | |
| Parietal Ctx | | | |
| Control | 1.4 | 1.3 | 2.9 |
| (Path) 3 | | | |
| Parietal Ctx | | | |
| Control | 30.8 | 40.1 | 38.2 |
| (Path) 4 | | | |
| Parietal Ctx | | | |

TABLE AGG
General_screening panel_v1.6

| | Rel. Exp. (%) |
|---|---|
| | Ag7017, Run |
| Tissue Name | 279032445 |
| Adipose | 0.1 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.1 |
| Prostate ca.* (bone met) PC-3 | 0.0 |
| Prostate Pool | 0.0 |
| Placenta | 0.2 |
| Uterus Pool | 0.0 |
| Ovarian ca. OVCAR-3 | 0.1 |
| Ovarian ca. SK-OV-3 | 0.0 |
| Ovarian ca. OVCAR-4 | 0.0 |
| Ovarian ca. OVCAR-5 | 0.1 |
| Ovarian ca. IGROV-1 | 0.8 |
| Ovarian ca. OVCAR-8 | 0.3 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.1 |
| Lung ca. NCI-N417 | 4.3 |
| Lung ca. LX-1 | 0.0 |
| Lung ca. NCI-H146 | 36.9 |
| Lung ca. SHP-77 | 28.5 |
| Lung ca. A549 | 0.0 |
| Lung ca. NCI-H526 | 27.9 |
| Lung ca. NCI-H23 | 0.1 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.1 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.1 |
| Colon ca.* (SW480 met) SW620 | 0.1 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.1 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.2 |
| CNS cancer (astro) SF-539 | 0.0 |
| CNS cancer (astro) SNB-75 | 0.1 |
| CNS cancer (glio) SNB-19 | 0.6 |
| CNS cancer (glio) SF-295 | 0.0 |
| Brain (Amygdala) Pool | 9.7 |
| Brain (cerebellum) | 84.7 |
| Brain (fetal) | 100.0 |
| Brain (Hippocampus) Pool | 12.8 |
| Cerebral Cortex Pool | 11.1 |
| Brain (Substantia nigra) Pool | 6.4 |
| Brain (Thalamus) Pool | 19.5 |
| Brain (whole) | 22.7 |
| Spinal Cord Pool | 2.1 |
| Adrenal Gland | 0.1 |
| Pituitary gland Pool | 1.8 |
| Salivary Gland | 0.0 |
| Thyroid (female) | 0.0 |
| Pancreatic ca. CAPAN2 | 0.0 |
| Pancreas Pool | 0.0 |

TABLE AGH
HASS Panel v1.0

| | Rel. Exp. (%) |
|---|---|
| | Ag2795, Run |
| Tissue Name | 268787250 |
| MCF-7 C1 | 0.2 |
| MCF-7 C2 | 0.0 |
| MCF-7 C3 | 0.4 |
| MCF-7 C4 | 0.0 |
| MCF-7 C5 | 0.2 |
| MCF-7 C6 | 0.5 |
| MCF-7 C7 | 0.4 |
| MCF-7 C9 | 0.2 |
| MCF-7 C10 | 0.1 |
| MCF-7 C11 | 0.0 |
| MCF-7 C12 | 0.2 |
| MCF-7 C13 | 0.3 |
| MCF-7 C15 | 0.2 |
| MCF-7 C16 | 0.3 |
| MCF-7 C17 | 0.4 |
| T24 D1 | 0.0 |
| T24 D2 | 0.1 |
| T24 D3 | 0.0 |
| T24 D4 | 0.0 |
| T24 D5 | 0.1 |
| T24 D6 | 0.0 |
| T24 D7 | 0.0 |
| T24 D9 | 0.0 |
| T24 D10 | 0.0 |
| T24 D11 | 0.0 |
| T24 D12 | 0.0 |
| T24 D13 | 0.0 |
| T24 D15 | 0.0 |
| T24 D16 | 0.0 |
| T24 D17 | 0.0 |
| CAPaN B1 | 0.0 |
| CAPaN B2 | 0.0 |
| CAPaN B3 | 0.0 |
| CAPaN B4 | 0.0 |
| CAPaN B5 | 0.0 |
| CAPaN B6 | 0.0 |
| CAPaN B7 | 0.0 |
| CAPaN B8 | 0.0 |
| CAPaN B9 | 0.1 |
| CAPaN B10 | 0.0 |
| CAPaN B11 | 0.1 |
| CAPaN B12 | 0.0 |
| CAPaN B13 | 0.0 |
| CAPaN B14 | 0.0 |
| CAPaN B15 | 0.0 |
| CAPaN B16 | 0.0 |
| CAPaN B17 | 0.0 |
| U87-MG F1 (B) | 0.0 |
| U87-MG F2 | 0.0 |
| U87-MG F3 | 0.0 |
| U87-MG F4 | 0.2 |
| U87-MG F5 | 0.0 |
| U87-MG F6 | 0.0 |
| U87-MG F7 | 0.1 |
| U87-MG F8 | 0.1 |
| U87-MG F9 | 0.0 |
| U87-MG F10 | 0.0 |
| U87-MG F11 | 0.0 |
| U87-MG F12 | 0.0 |
| U87-MG F13 | 0.0 |
| U87-MG F14 | 0.0 |
| U87-MG F15 | 0.1 |
| U87-MG F16 | 0.0 |
| U87-MG F17 | 0.0 |
| LnCAP A1 | 0.2 |
| LnCAP A2 | 0.5 |
| LnCAP A3 | 0.2 |
| LnCAP A4 | 0.9 |
| LnCAP A5 | 0.1 |
| LnCAP A6 | 0.3 |
| LnCAP A7 | 0.4 |
| LnCAP A8 | 0.2 |
| LnCAP A9 | 0.0 |
| LnCAP A10 | 0.3 |
| LnCAP A11 | 0.9 |
| LnCAP A12 | 0.0 |
| LnCAP A13 | 0.0 |
| LnCAP A14 | 0.0 |
| LnCAP A15 | 0.1 |
| LnCAP A16 | 0.9 |
| LnCAP A17 | 0.2 |
| Primary Astrocytes | 1.0 |
| Primary Renal Proximal | 0.0 |
| Tubule Epithelial cell A2 | |
| Primary melanocytes A5 | 0.0 |
| 126443 - 341 medullo | 0.0 |
| 126444 - 487 medullo | 0.1 |
| 126445 - 425 medullo | 0.5 |
| 126446 - 690 medullo | 100.0 |
| 126447 - 54 adult glioma | 0.3 |
| 126448 - 245 adult glioma | 0.1 |
| 126449 - 317 adult glioma | 3.7 |
| 126450 - 212 glioma | 30.8 |
| 126451 - 456 glioma | 50.0 |

TABLE AGI
Oncology_cell_line_screening_panel_v3.2

| | Rel.Exp. (%) |
|---|---|
| | Ag2795, Run |
| Tissue Name | 271400611 |
| 94905_Daoy_Medulloblastoma/ | 0.0 |
| Cerebellum_sscDNA | |
| 94906_TE671_Medulloblastom/ | 0.0 |
| Cerebellum_sscDNA | |
| 94907_D283 Med_Medulloblastoma/ | 7.2 |
| Cerebellum sscDNA | |
| 94908_PFSK-1_Primitive | 0.0 |
| Neuroectodermal/Cerebellum sscDNA | |
| 94909_XF-498_CNS_sscDNA | 0.2 |
| 94910_SNB-78_CNS/glioma_sscDNA | 0.0 |
| 94911_SF-268_CNS/ | 0.0 |
| glioblastoma_sscDNA | |
| 94912_T98G_Glioblastoma_sscDNA | 0.0 |
| 96776_SK-N-SH_Neuroblastoma | 0.6 |
| (metastasis)_sscDNA | |
| 94913_SF- | 0.0 |
| 295_CNS/glioblastoma_sscDNA | |
| 132565_NT2 pool_sscDNA | 0.0 |
| 94914_Cerebellum_sscDNA | 14.9 |
| 96777_Cerebellum_sscDNA | 15.2 |
| 94916_NCI-H292_Mucoepidermoid lung | 0.0 |
| carcinoma_sscDNA | |
| 94917_DMS-114_Small cell lung | 0.0 |
| cancer_sscDNA | |
| 94918_DMS-79_Small cell lung | 100.0 |
| cancer/neuroendocrine_sscDNA | |
| 94919_NCI-H146_Small cell lung | 36.6 |
| cancer/neuroendocrine_sscDNA | |
| 94920_NCI-H526_Small cell lung | 33.0 |
| cancer/neuroendocrine_sscDNA | |
| 94921_NCI-N417_Small cell lung | 5.1 |
| cancer/neuroendocrine_sscDNA | |
| | 6.9 |
| 94923_NCI-H82_Small cell lung | |
| cancer/neuroendocrine_sscDNA | |
| 94924_NCI-H157_Squamous cell lung | 0.0 |
| cancer (metastasis)_sscDNA | |
| 94925_NCI-H1155_Large cell lung | 7.3 |
| cancer/neuroendocrine_sscDNA | |
| 94926_NCI-H1299_Large cell lung | 0.0 |
| cancer/neuroendocrine_sscDNA | |
| 94927_NCI-H727_Lung | 3.1 |
| carcinoid_sscDNA | |
| 94928_NCI-UMC-11_Lung | 7.5 |
| carcinoid_sscDNA | |
| 94929_LX-1_Small cell lung | 0.0 |
| cancer_sscDNA | |
| 94930_Colo-205_Colon cancer_sscDNA | 0.0 |
| 94931_KM12_Colon cancer_sscDNA | 0.0 |
| 94932 KM20L2_Colon cancer_sscDNA | 0.0 |
| 94933_NCI-H716_Colon cancer_sscDNA | 3.6 |
| 94935_SW-48_Colon | 0.0 |
| adenocarcinoma_sscDNA | |
| 94936_SW1116_Colon | 0.0 |
| adenocarcinoma_sscDNA | |
| 94937_LS 174T_Colon | 0.0 |
| adenocarcinoma_sscDNA | |
| 94938_SW-948_Colon | 0.0 |
| adenocarcinoma_sscDNA | |
| 94939_SW-480_Colon | 0.0 |
| adenocarcinoma_sscDNA | |
| 94940_NCI-SNU-5_Gastric | 0.0 |
| carcinoma_sscDNA | |
| 112197_KATO III_Stomach_sscDNA | 0.0 |
| 94943_NCI-SNU-16_Gastric | 0.0 |
| carcinoma_sscDNA | |
| 94944_NCI-SNU-1_Gastric | 0.0 |
| carcinoma_sscDNA | |
| 94946_RF-1_Gastric | 0.0 |
| adenocarcinoma_sscDNA | |
| 94947_RF-48_Gastric | 0.0 |
| adenocarcinoma_sscDNA | |
| 96778_MKN-45_Gastric | 0.0 |
| carcinoma_sscDNA | |
| 94949_NCI-N87_Gastric | 0.0 |
| carcinoma_sscDNA | |
| 94951_OVCAR-5_Ovarian | 0.0 |
| carcinoma_sscDNA | |
| 94952_RL95-2_Uterine | 0.0 |
| carcinoma_sscDNA | |
| 94953_HelaS3_Cervical | 0.0 |
| adenocarcinoma_sscDNA | |
| 94954_Ca Ski_Cervical epidermoid | 0.0 |
| carcinoma (metastasis)_sscDNA | |
| 94955_ES-2_Ovarian clear cell | 0.0 |
| carcinoma_sscDNA | |
| 94957_Ramos/6 h stim_Stimulated | 0.0 |
| with PMA/ionomycin 6 h_sscDNA | |
| 94958_Ramos/14 h stim_Stimulated | 0.0 |
| with PMA/ionomycin 14 h_sscDNA | |
| 94962_MEG-01_Chronic myelogenous | 0.0 |
| leukemia (megokaryoblast)_sscDNA | |
| 94963_Raji_Burkitt's | 0.0 |
| lymphoma_sscDNA | |
| 94964_Daudi_Burkitt's | 0.0 |
| lymphoma_sscDNA | |
| 94965_U266_B-cell | 0.0 |
| plasmacytoma/myeloma_sscDNA | |
| 94968_CA46_Burkitt's | 0.0 |
| lymphoma_sscDNA | |
| 94970_RL_non-Hodgkin's B-cell | 0.0 |
| lymphoma_sscDNA | |
| 94972_JM1_pre-B-cell | 0.0 |
| lymphoma/leukemia_sscDNA | |
| 94973_Jurkat_T cell | 0.0 |
| leukemia_sscDNA | |
| 94974_TF- | 0.0 |
| 1_Erythroleukemia_sscDNA | |
| 94975_HUT 78_T-cell | 0.0 |
| lymphoma_sscDNA | |
| 94977_U937_Histiocytic | 0.0 |
| lymphoma_sscDNA | |
| 94980_KU-812_Myelogenous | 0.0 |
| leukemia_sscDNA | |
| 94981_769-P_Clear cell renal | 0.0 |
| carcinoma_sscDNA | |
| 94983_Caki-2_Clear cell renal | 0.0 |
| carcinoma_sscDNA | |
| 94984_SW 839_Clear cell renal | 0.0 |
| carcinoma_sscDNA | |
| 94986_G401_Wilms' tumor_sscDNA | 0.0 |
| 126768_293 cells_sscDNA | 0.0 |
| 94987_Hs766T_Pancreatic | 0.0 |
| carcinoma (LN metastasis)_sscDNA | |
| 94988_CAPAN-1_Pancreatic | 0.0 |
| adenocarcinoma (liver | |
| metastasis)_sscDNA | |
| 94989_SU86.86_Pancreatic | 0.0 |
| carcinoma (liver | |
| metastasis)_sscDNA | |
| 94990_BxPC-3_Pancreatic | 0.0 |
| adenocarcinoma_sscDNA | |
| 94991_HPAC_Pancreatic | 0.0 |
| adenocarcinoma_sscDNA | |
| 94992_M1A PaCa-2_Pancreatic | 0.0 |
| carcinoma_sscDNA | |
| 94993_CFPAC-1_Pancreatic ductal | 0.0 |
| adenocarcinoma_sscDNA | |
| 94994_PANC-1_Pancreatic epithelioid | 0.0 |
| ductal carcinoma_sscDNA | |
| 94996_T24_Bladder carcinma | 0.0 |
| (transitional cell)_sscDNA | |
| 94997_5637_Bladder | 0.0 |
| carcinoma_sscDNA | |
| 94998_HT-1197_Bladder | 0.0 |
| carcinoma_sscDNA | |
| 94999_UM-UC-3_Bladder carcinma | 0.0 |
| (transitional cell)_sscDNA | |
| 95000_A204_Rhabdomyosarcoma_sscDNA | 0.0 |
| 95001_HT-1080_Fibrosarcoma_sscDNA | 0.0 |
| 95002_MG-63_Osteosarcoma | 0.0 |
| (bone)_sscDNA | |
| 95003_SK-LMS-1_Leiomyosarcoma | 0.0 |
| (vulva)_sscDNA | |
| 95004_SJRH30_Rhabdomyosarcoma | 0.8 |
| (met to bone marrow)_sscDNA | |
| 95005_A431_Epidermoid | 0.0 |
| carcinoma_sscDNA | |
| 95007_WM266-4_Melanoma_sscDNA | 0.0 |
| 112195_DU 145_Prostate_sscDNA | 0.0 |
| 95012_MDA-MB-468_Breast | 0.0 |
| adenocarcinoma_sscDNA | |
| 112196_SSC-4_Tongue_sscDNA | 0.0 |
| 112194_SSC-9_Tongue_sscDNA | 0.0 |
| 112191_SSC-15_Tongue_sscDNA | 0.0 |
| 95017_CAL 27_Squamous cell | 0.0 |
| carcinoma of tongue_sscDNA | |

TABLE AGJ
Panel 1

| | Rel. Exp. (%) |
|---|---|
| | Ag90, Run |
| Tissue Name | 87586258 |
| Endothelial cells | 0.0 |
| Endothelial cells (treated) | 0.0 |
| Pancreas | 0.1 |
| Pancreatic ca. CAPAN 2 | 0.0 |
| Adrenal gland | 0.0 |
| Thyroid | 0.0 |
| Salivary gland | 0.0 |
| Pituitary gland | 0.0 |
| Brain (fetal) | 37.1 |
| Brain (whole) | 22.5 |
| Brain (amygdala) | 24.8 |
| Brain (cerebellum) | 100.0 |
| Brain (hippocampus) | 29.5 |
| Brain (substantia nigra) | 7.6 |
| Brain (thalamus) | 13.7 |
| Brain (hypothalamus) | 7.7 |
| Spinal cord | 1.4 |
| glio/astro U87-MG | 0.0 |
| glio/astro U-118-MG | 0.0 |
| astrocytoma SW1783 | 0.0 |
| neuro*; met SK-N-AS | 0.4 |
| astrocytoma SF-539 | 0.0 |
| astrocytoma SNB-75 | 0.0 |
| glioma SNB-19 | 1.8 |
| glioma U251 | 0.4 |
| glioma SF-295 | 0.0 |
| Heart | 0.0 |
| Skeletal muscle | 0.0 |
| Bone marrow | 0.0 |
| Thymus | 0.1 |
| Spleen | 0.0 |
| Lymph node | 0.0 |
| Colon (ascending) | 0.1 |
| Stomach | 0.1 |
| Small intestine | 0.3 |
| Colon ca. SW480 | 0.0 |
| Colon ca.* SW620 (SW480 met) | 0.0 |
| Colon ca. HT29 | 0.0 |
| Colon ca. HCT-116 | 0.0 |
| Colon ca. CaCo-2 | 0.0 |
| Colon ca. HCT-15 | 0.0 |
| Colon ca. HCC-2998 | 0.0 |
| Gastric ca. * (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 0.0 |
| Renal ca. A498 | 0.0 |
| Renal ca. RXF 393 | 0.0 |
| Renal ca. ACHN | 0.0 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 33.7 |
| Lung ca. (s. cell var.) SHP-77 | 0.0 |
| Lung ca. (large cell) NCI-H460 | 0.0 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.0 |
| Lung ca. (squam.) NCI-H596 | 20.0 |
| Mammary gland | 0.1 |
| Breast ca.* (pl. ef) MCF-7 | 0.0 |
| Breast ca.* (pl. ef) MDA-MB-231 | 0.0 |
| Breast ca.* (pl. ef) T47D | 0.0 |
| Breast ca. BT-549 | 0.0 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.0 |
| Ovarian ca. OVCAR-8 | 0.0 |
| Ovarian ca. IGROV-1 | 0.0 |
| Ovarian ca. (ascites) SK-OV-3 | 0.0 |
| Uterus | 0.0 |
| Placenta | 0.0 |
| Prostate | 0.0 |
| Prostate ca.* (bone met) PC-3 | 0.0 |
| Testis | 1.3 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMV1 | 0.0 |
| Melanoma* (met) SK-MEL-5 | 0.0 |
| Melanoma SK-MEL-28 | 0.0 |

TABLE AGK
Panel 1.3D

| | Rel. Exp. (%) | Rel. Exp. (%) |
|---|---|---|
| | Ag2795, Run | Ag2807, Run |
| Tissue Name | 165643063 | 165528058 |
| Liver adenocarcinoma | 0.0 | 0.0 |
| Pancreas | 0.0 | 0.2 |
| Pancreatic ca. CAPAN 2 | 0.0 | 0.0 |
| Adrenal gland | 0.0 | 0.3 |
| Thyroid | 0.0 | 0.0 |
| Salivary gland | 0.0 | 0.0 |
| Pituitary gland | 6.8 | 6.9 |
| Brain (fetal) | 100.0 | 100.0 |
| Brain (whole) | 51.4 | 56.3 |
| Brain (amygdala) | 77.4 | 78.5 |
| Brain (cerebellum) | 58.6 | 79.0 |
| Brain (hippocampus) | 49.0 | 53.2 |
| Brain (substantia nigra) | 9.7 | 13.5 |
| Brain (thalamus) | 46.7 | 63.7 |
| Cerebral Cortex | 37.9 | 32.5 |
| Spinal cord | 5.9 | 4.1 |
| glio/astro U87-MG | 0.0 | 0.0 |
| glio/astro U-118-MG | 0.0 | 0.0 |
| astrocytoma SW1783 | 0.0 | 0.1 |
| neuro*; met SK-N-AS | 0.4 | 0.0 |
| astrocytoma SF-539 | 0.0 | 0.0 |
| astrocytoma SNB-75 | 0.0 | 0.0 |
| glioma SNB-19 | 1.2 | 2.5 |
| glioma U251 | 3.5 | 4.2 |
| glioma SF-295 | 0.0 | 0.0 |
| Heart (fetal) | 0.0 | 0.0 |
| Heart | 0.1 | 0.0 |
| Skeletal muscle (fetal) | 0.4 | 0.0 |
| Skeletal muscle | 0.0 | 0.0 |
| Bone marrow | 0.0 | 0.0 |
| Thymus | 0.0 | 0.0 |
| Spleen | 0.5 | 0.0 |
| Lymph node | 0.1 | 0.1 |
| Colorectal | 0.0 | 0.0 |
| Stomach | 0.0 | 0.0 |
| Small intestine | 0.6 | 1.0 |
| Colon ca. SW480 | 0.0 | 0.0 |
| Colon ca.* SW620 (SW480 met) | 0.0 | 0.0 |
| Colon ca. HT29 | 0.0 | 0.0 |
| Colon ca. HCT-116 | 0.3 | 0.0 |
| Colon ca. CaCo-2 | 0.0 | 0.0 |
| Colon ca. tissue (ODO3866) | 0.0 | 0.0 |
| Colon ca. HCC-2998 | 0.0 | 0.0 |
| Gastric ca.* (liver | 0.4 | 0.1 |
| met) NCI-N87 | | |
| Bladder | 0.4 | 0.1 |
| Trachea | 0.1 | 0.1 |
| Kidney | 0.0 | 0.0 |
| Kidney (fetal) | 0.0 | 0.0 |
| Renal ca. 786-0 | 0.0 | 0.0 |
| Renal ca. A498 | 0.3 | 0.3 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.2 | 0.0 |
| Liver (fetal) | 0.0 | 0.0 |
| Liver ca. | 0.0 | 0.0 |
| (hepatoblast) HepG2 | | |
| Lung | 0.0 | 0.0 |
| Lung (fetal) | 0.1 | 0.2 |
| Lung ca. (small cell) LX-1 | 0.0 | 0.0 |
| Lung ca. (small cell) NCI-H69 | 41.5 | 92.7 |
| Lung ca. (s.cell var.) SHP-77 | 34.4 | 25.2 |
| Lung ca. (large | 0.3 | 0.0 |
| cell) NCI-H460 | | |
| Lung ca. (non-sm. cell) A549 | 0.0 | 0.0 |
| Lung ca. (non-s.cell) NCI-H23 | 0.1 | 0.0 |
| Lung ca. (non-s.cell) HOP-62 | 0.0 | 0.0 |
| Lung ca. (non-s.cl) NCI-H522 | 0.0 | 0.0 |
| Lung ca. (squam.) SW 900 | 0.0 | 0.0 |
| Lung ca. (squam.) NCI-H596 | 70.2 | 58.2 |
| Mammary gland | 0.4 | 1.1 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 0.0 | 0.0 |
| Breast ca. MDA-N | 0.0 | 0.0 |
| Ovary | 0.0 | 0.0 |
| Ovarian ca. OVCAR-3 | 0.0 | 0.2 |
| Ovarian ca. OVCAR-4 | 0.1 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca.* | 0.2 | 0.0 |
| (ascites) SK-OV-3 | | |
| Uterus | 0.0 | 0.1 |
| Placenta | 0.1 | 0.2 |
| Prostate | 0.0 | 0.0 |
| Prostate ca.* (bone | 0.0 | 0.0 |
| met) PC-3 | | |
| Testis | 0.4 | 0.3 |
| Melanoma Hs688(A).T | 0.0 | 0.0 |
| Melanoma* (met) Hs688(B).T | 0.1 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.1 |
| Adipose | 0.1 | 0.0 |

TABLE AGL
Panel 2D

| | Rel. Exp. (%) | Rel. Exp. (%) |
|---|---|---|
| | Ag2795, | Ag2807, |
| | Run | Run |
| Tissue Name | 163577802 | 162598819 |
| Normal Colon | 6.3 | 13.2 |
| CC Well to Mod | 0.0 | 0.0 |
| Diff (ODO3866) | | |
| CC Margin (ODO3866) | 1.7 | 5.9 |
| CC Gr.2 rectosigmoid | 0.0 | 0.0 |
| (ODO3868) | | |
| CC Margin (ODO3868) | 0.0 | 2.9 |
| CC Mod Diff (ODO3920) | 0.0 | 0.7 |
| CC Margin (ODO3920) | 0.6 | 1.4 |
| CC Gr.2 ascend | 0.0 | 3.4 |
| colon (ODO3921) | | |
| CC Margin (ODO3921) | 1.0 | 2.3 |
| CC from Partial Hepatectomy | 0.0 | 0.4 |
| (ODO4309) Mets | | |
| Liver Margin (ODO4309) | 0.0 | 0.4 |
| Colon mets to lung | 0.0 | 2.9 |
| (OD04451-01) | | |
| Lung Margin (OD04451-02) | 1.1 | 4.5 |
| Normal Prostate 6546-1 | 6.7 | 9.8 |
| Prostate Cancer (OD04410) | 2.3 | 3.4 |
| Prostate Margin (OD04410) | 1.6 | 3.3 |
| Prostate Cancer (OD04720-01) | 1.2 | 2.6 |
| Prostate Margin (OD04720-02) | 9.0 | 7.2 |
| Normal Lung 061010 | 2.1 | 2.6 |
| Lung Met to Muscle (ODO4286) | 0.0 | 0.0 |
| Muscle Margin (ODO4286) | 0.7 | 0.0 |
| Lung Malignant | 0.0 | 0.0 |
| Cancer (OD03126) | | |
| Lung Margin (OD03126) | 0.5 | 0.5 |
| Lung Cancer (OD04404) | 0.0 | 0.0 |
| Lung Margin (OD04404) | 0.0 | 1.5 |
| Lung Cancer (OD04565) | 0.0 | 0.3 |
| Lung Margin (OD04565) | 0.0 | 1.7 |
| Lung Cancer (OD04237-01) | 20.4 | 13.2 |
| Lung Margin (OD04237-02) | 1.3 | 0.9 |
| Ocular Mel Met to | 0.0 | 2.6 |
| Liver (ODO4310) | | |
| Liver Margin (OD04310) | 0.0 | 0.0 |
| Melanoma Mets to | 0.0 | 0.0 |
| Lung (OD04321) | | |
| Lung Margin (OD04321) | 1.6 | 2.9 |
| Normal Kidney | 1.1 | 1.0 |
| Kidney Ca, Nuclear | 1.2 | 1.2 |
| grade 2 (OD04338) | | |
| Kidney Margin (OD04338) | 0.5 | 1.1 |
| Kidney Ca Nuclear | 0.0 | 0.5 |
| grade 1/2 (OD04339) | | |
| Kidney Margin (OD04339) | 0.5 | 0.9 |
| Kidney Ca, Clear | 0.0 | 0.5 |
| cell type (OD04340) | | |
| Kidney Margin (OD04340) | 1.0 | 0.3 |
| Kidney Ca, Nuclear | 0.0 | 0.9 |
| grade 3 (OD04348) | | |
| Kidney Margin (OD04348) | 0.3 | 0.8 |
| Kidney Cancer (OD04622-01) | 0.0 | 0.9 |
| Kidney Margin (OD04622-03) | 0.0 | 0.0 |
| Kidney Cancer (OD04450-01) | 0.0 | 1.2 |
| Kidney Margin (OD04450-03) | 0.3 | 0.4 |
| Kidney Cancer 8120607 | 0.0 | 0.0 |
| Kidney Margin 8120608 | 0.0 | 3.8 |
| Kidney Cancer 8120613 | 0.5 | 0.0 |
| Kidney Margin 8120614 | 2.5 | 1.5 |
| Kidney Cancer 9010320 | 0.0 | 0.7 |
| Kidney Margin 9010321 | 1.7 | 1.7 |
| Normal Uterus | 0.0 | 1.0 |
| Uterus Cancer 064011 | 1.5 | 0.8 |
| Normal Thyroid | 1.3 | 0.3 |
| Thyroid Cancer 064010 | 1.6 | 0.0 |
| Thyroid Cancer A302152 | 1.3 | 0.2 |
| Thyroid Margin A302153 | 0.0 | 0.0 |
| Normal Breast | 0.7 | 1.7 |
| Breast Cancer (OD04566) | 0.0 | 0.3 |
| Breast Cancer (OD04590-01) | 0.9 | 1.3 |
| Breast Cancer Mets | 0.6 | 1.8 |
| (OD04590-03) | | |
| Breast Cancer Metastasis | 1.0 | 0.8 |
| (OD04655-05) | | |
| Breast Cancer 064006 | 0.0 | 0.0 |
| Breast Cancer 1024 | 0.0 | 1.5 |
| Breast Cancer 9100266 | 0.3 | 1.2 |
| Breast Margin 9100265 | 0.0 | 1.7 |
| Breast Cancer A209073 | 0.3 | 0.0 |
| Breast Margin A209073 | 0.0 | 0.0 |
| Normal Liver | 0.0 | 0.4 |
| Liver Cancer 064003 | 0.0 | 0.0 |
| Liver Cancer 1025 | 0.0 | 0.0 |
| Liver Cancer 1026 | 100.0 | 100.0 |
| Liver Cancer 6004-T | 0.0 | 0.0 |
| Liver Tissue 6004-N | 0.0 | 0.0 |
| Liver Cancer 6005-T | 80.1 | 64.6 |
| Liver Tissue 6005-N | 0.0 | 0.0 |
| Normal Bladder | 7.2 | 7.4 |
| Bladder Cancer 1023 | 0.4 | 0.0 |
| Bladder Cancer A302173 | 1.3 | 2.8 |
| Bladder Cancer (OD04718-01) | 0.0 | 0.4 |
| Bladder Normal | 0.7 | 0.4 |
| Adjacent (OD04718-03) | | |
| Normal Ovary | 0.0 | 0.0 |
| Ovarian Cancer 064008 | 0.0 | 0.7 |
| Ovarian Cancer (OD04768-07) | 0.4 | 1.1 |
| Ovary Margin (OD04768-08) | 0.0 | 0.0 |
| Normal Stomach | 1.2 | 2.6 |
| Gastric Cancer 9060358 | 0.0 | 0.0 |
| Stomach Margin 9060359 | 0.4 | 1.4 |
| Gastric Cancer 9060395 | 0.4 | 0.5 |
| Stomach Margin 9060394 | 0.6 | 2.0 |
| Gastric Cancer 9060397 | 1.4 | 0.8 |
| Stomach Margin 9060396 | 6.0 | 1.9 |
| Gastric Cancer 064005 | 3.4 | 1.6 |

TABLE AGM
Panel 4.1D

| | Rel. Exp. (%) |
|---|---|
| | Ag7017, Run |
| Tissue Name | 279031713 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 2.1 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ryTh1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 5.5 |
| EOL-1 dbcAMP | 13.6 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 14.3 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1 beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 42.0 |
| Astrocytes TNFalpha + IL-1beta | 15.5 |
| KU-8l2 (Basophil) rest | 0.0 |
| KU-8l2 (Basophil) PMA/ionomycin | 0.0 |
| CCD1106 (Keratinocytes) none | 0.0 |
| CCD1106 (Keratinocytes) TNFalpha + IL-1 beta | 0.0 |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 5.3 |
| NCI-H292 IFN gamma | 0.0 |
| HPAEC none | 0.0 |
| HPAEC TNF alpha + IL-1beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 100.0 |
| Neutrophils rest | 0.0 |
| Colon | 6.8 |
| Lung | 0.0 |
| Thymus | 0.0 |
| Kidney | 4.1 |

TABLE AGN
Panel 4D

| | Rel. Exp. (%) |
|---|---|
| | Ag2807, Run |
| Tissue Name | 165806333 |
| Secondary Th1 act | 1.2 |
| Secondary Th2 act | 1.1 |
| Secondary Tr1 act | 2.7 |
| Secondary Th1 rest | 1.9 |
| Secondary Th2 rest | 2.4 |
| Secondary Tr1 rest | 0.0 |
| Primary Th1 act | 0.0 |
| Primary Th2 act | 1.2 |
| Primary Tr1 act | 0.0 |
| Primary Th1 rest | 6.1 |
| Primary Th2 rest | 2.7 |
| Primary Tr1 rest | 1.1 |
| CD45RA CD4 lymphocyte act | 4.0 |
| CD45RO CD4 lymphocyte act | 4.5 |
| CD8 lymphocyte act | 3.2 |
| Secondary CD8 lymphocyte rest | 2.1 |
| Secondary CD8 lymphocyte act | 1.6 |
| CD4 lymphocyte none | 1.2 |
| 2ry Th1/Th2/Tr1_anti-CD95 CH11 | 5.0 |
| LAK cells rest | 0.0 |
| LAK cells IL-2 | 4.1 |
| LAK cells IL-2 + IL-12 | 2.6 |
| LAK cells IL-2 + IFN gamma | 3.8 |
| LAK cells IL-2 + IL-18 | 1.0 |
| LAK cells PMA/ionomycin | 1.2 |
| NK Cells IL-2 rest | 2.1 |
| Two Way MLR 3 day | 3.8 |
| Two Way MLR 5 day | 2.6 |
| Two Way MLR 7 day | 3.6 |
| PBMC rest | 1.9 |
| PBMC PWM | 1.3 |
| PBMC PHA-L | 0.0 |
| Ramos (B cell) none | 6.0 |
| Ramos (B cell) ionomycin | 0.0 |
| B lymphocytes PWM | 0.9 |
| B lymphocytes CD40L and IL-4 | 9.0 |
| EOL-1 dbcAMP | 5.6 |
| EOL-1 dbcAMP PMA/ionomycin | 14.8 |
| Dendritic cells none | 4.3 |
| Dendritic cells LPS | 5.0 |
| Dendritic cells anti-CD40 | 5.1 |
| Monocytes rest | 5.4 |
| Monocytes LPS | 4.4 |
| Macrophages rest | 1.6 |
| Macrophages LPS | 0.0 |
| HUVEC none | 1.2 |
| HUVEC starved | 0.9 |
| HUVEC IL-1beta | 0.0 |
| HUVEC IFN gamma | 2.1 |
| HUVEC TNF alpha + IFN gamma | 0.0 |
| HUVEC TNF alpha + IL4 | 0.0 |
| HUVEC IL-11 | 0.9 |
| Lung Microvascular EC none | 1.0 |
| Lung Microvascular ECTNFalpha + IL-1beta | 0.0 |
| Microvascular Dermal EC none | 1.9 |
| Microsvasular Dermal EC TNFalpha + IL-1beta | 0.0 |
| Bronchial epithelium TNFalpha + IL1beta | 0.0 |
| Small airway epithelium none | 3.9 |
| Small airway epithelium TNFalpha + IL-1beta | 3.6 |
| Coronery artery SMC rest | 0.8 |
| Coronery artery SMC TNFalpha + IL-1beta | 0.0 |
| Astrocytes rest | 100.0 |
| Astrocytes TNFalpha + IL-1beta | 84.1 |
| KU-812 (Basophil) rest | 4.6 |
| KU-812 (Basophil) PMA/ionomycin | 1.1 |
| CCD1106 (Keratinocytes) none | 0.0 |
| CCD1106 (Keratinocytes) TNFalpha + IL-1beta | 0.0 |
| Liver cirrhosis | 1.1 |
| Lupus kidney | 1.6 |
| NCI-H292 none | 6.1 |
| NCI-H292 IL-4 | 2.5 |
| NCI-H292 IL-9 | 2.1 |
| NCI-H292 IL-13 | 2.8 |
| NCI-H292 IFN gamma | 1.0 |
| HPAEC none | 0.0 |
| HPAEC TNF alpha + IL-1 beta | 0.0 |
| Lung fibroblast none | 1.3 |
| Lung fibroblast TNF alpha + IL-1 beta | 1.6 |
| Lung fibroblast IL-4 | 2.5 |
| Lung fibroblast IL-9 | 2.8 |
| Lung fibroblast IL-13 | 1.3 |
| Lung fibroblast IFN gamma | 0.0 |
| Dermal fibroblast CCD1070 rest | 2.1 |
| Dermal fibroblast CCD1070 TNF alpha | 4.4 |
| Dermal fibroblast CCD1070 IL-1 beta | 0.8 |
| Dermal fibroblast IFN gamma | 1.5 |
| Dermal fibroblast IL-4 | 0.0 |
| IBD Colitis 2 | 6.3 |
| IBD Crohn's | 0.0 |
| Colon | 58.2 |
| Lung | 3.4 |
| Thymus | 8.8 |
| Kidney | 31.6 |

AI_comprehensive panel_v1.0 Summary: Ag2795 High expression of this gene is mostly restricted to orthoarthritis (OA) bone (CT=28). Thus, expression of this gene may be used to distinguish OA bone from other samples used in this panel. In addition, therapeutic modulation of this gene product may be useful in the treatment of orthoarthritis.

CNS_neurodegeneration_v1.0 Summary: Ag2795/Ag2807/Ag7017 Three experiments with two different probes and primer sets are in very good agreement. This panel confirms the expression of this gene at low levels in the brains of an independent group of individuals. However, no differential expression of this gene was detected between Alzheimer's diseased postmortem brains and those of non-demented controls in this experiment. See Panel 1.3D for a discussion of this gene in treatment of central nervous system disorders.

General_screening_panel_v1.6 Summary: Ag7017 Highest expression of this gene is detected in brain cerebellum (CT=25.3). High to moderate levels of expression of this gene is mainly seen in all the regions of brain including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

This gene codes for a homolog of mouse seizure related protein, SEZ-6. Mouse SEZ-6 was first isolated from cerebrum cortex-derived cells treated with pentylentetrazole (PTZ), one of the convulsant drugs (Shimizu-Nishikawa et al., 1995, Brain Res Mol Brain Res 28(2):201-10, PMID: 7723619). Thus, SEZ-6 protein encoded by this gene may also play a role in brain seizure.

In addition, moderate to low levels of expression of this gene is also seen in four lung cancer cell lines and a ovarian cancer cell line. Therefore, expression of this gene may be used as diagnostic marker to detect lung cancer and also, modulation of this gene or its protein product through the use of antibody or protein therapeutics, may be useful in the treatment of lung and ovarian cancers.

HASS Panel v1.0 Summary: Ag7017 Highest expression of this gene is detected in a medulloblastoma (CT=28). In addition, moderate levels of expression of this gene is also seen in glioma samples. Therefore, therapeutic modulation of this gene may be useful in the treatment of brain cancer.

Oncology_cell_line_screening_panel_v3.2 Summary: Ag2795 Highest expression of this gene is detected in small lung cancer DMS-79 cell line (CT=26.5). Moderate to low levels of expression of this gene is also seen in number of cell lines derived from lung, colon, bone and brain cancers. Therefore, expression of this gene may be used as marker to detect these cancers. In addition, therapeutic modulation of this gene through the use of antibodies or small molecule drug may be useful in the treatment of lung, colon, bone and brain cancers.

Panel 1 Summary: Ag90 Highest expression of this gene is detected in brain cerebellum (CT=25). High levels of expression of this gene is mainly seen in all the regions of brain including amygdala, hippocampus, substantia nigra, thalamus, cerebellum, cerebral cortex, and spinal cord. In addition, moderate levels of expression of this gene is also seen in two lung cancer cell lines and a glioma cell line. See panel 1.3D for further discussion of this gene.

Panel 1.3D Summary: Ag2795/Ag2807 Two experiments with same probe and primer sets are in excellent agreement with highest expression of this gene detected fetal brain (CTs=27-28.5). Moderate levels of expression of this gene is mainly seen in all the regions of brain including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

This gene codes for a homolog of mouse seizure related protein, SEZ-6. Mouse SEZ-6, was first isolated from cerebrum cortex-derived cells treated with pentylentetrazole (PTZ), one of the convulsant drugs (Shimizu-Nishikawa et al., 1995, Brain Res Mol Brain Res 28(2):201-10, PMID: 7723619). Thus, SEZ-6 protein encoded by this gene may also play a role in brain seizure.

In addition, moderate to low levels of expression of this gene is also seen in three lung cancer cell lines and two of the glioma cell lines. Therefore, expression of this gene may be used as diagnostic marker to detect lung cancer and glioma. Furthermore, modulation of this gene or its protein product through the use of antibody or protein therapeutics, may be useful in the treatment of lung cancer and glioma.

Panel 2D Summary: Ag2795/Ag2807 Two experiments with same probe and primer sets are in excellent agreement with highest expression of this gene detected in liver cancer 1026 sample (CTs=31.3). In addition, moderate to low levels of expression of this gene is also seen in a lung cancer and a liver cancer (6005-T). Expression of this gene is higher in cancer as compared to corresponding adjacent normal tissue (CTs>37). Thus, expression of this gene may be used to distinguish between normal and cancer samples and as diagnostic marker to detect lung and liver cancer. In addition, therapeutic modulation of this gene through the use of antibodies may be useful in the treatment of these cancers.

Panel 4.1D Summary: Ag7017 Low levels of expression of this gene is restricted to TNF alpha and LPS stimulated neutrophils (CT=34.4). Therefore, expression of this gene may be used to distinguish this sample from other samples in the panel. This expression profile suggest that the protein encoded by this gene is produced by activated neutrophils but not by resting neutrophils. Therefore, therapeutic modulation of this gene product through the use of antibodies or small molecule drug may reduce activation of these inflammatory cells and be useful to reduce or eliminate the symptoms in patients with Crohn's disease, ulcerative colitis, multiple sclerosis, chronic obstructive pulmonary disease, asthma, emphysema, rheumatoid arthritis, lupus erythematosus, or psoriasis. In addition, small molecule or antibody antagonists of this gene product may be effective in increasing the immune response in patients with AIDS or other immunodeficiencies.

Panel 4D Summary: Ag2807 Highest expression of this gene is detected in astrocytes (CTs=33). Thus expression of this gene may be used to distinguish astrocytes from other samples in this panel. In addition, low but significant levels of expression of this gene is also seen in normal tissue represented by colon and kidney. Therefore, therapeutic modulation of this gene may be useful in the treatment of autoimmune and inflammatory diseases affecting brain, colon and kidney such as lupus erythematosus, Crohn's disease, and ulcerative colitis.

general oncology screening panel_v_{—}2.4 Summary: Ag2795 Expression of this gene is low/undetectable (CTs>35) across all of the samples on this panel (data not shown).

AH. CG52919-05 and CG52919-06: SEZ-6-Like Protein (7520500-54-4).

Expression of gene CG52919-05 and CG52919-06 was assessed using the primer-probe sets Ag2796, Ag90 and Ag124, described in Tables AHA, AHB and AHC. Results of the RTQ-PCR runs are shown in Tables AHD, AHE, AHF and AHG. Note that probe-primer sets Ag2796 and Ag124 are specific for the CG52919-05 variant. Also, Note that CG52919-06 represents a full-length physical clone.

TABLE AHA

Probe Name Ag2796

| | | | Start | |
|---|---|---|---|---|
| Primers | Sequences | Length | Position | SEQ ID No |
| Forward | 5′-cctacaaccgcattaccataga-3′ | 22 | 1670 | 399 |
| Probe | TET-5′-tcagcgtttgacaatccaacttacga-3′-TAMRA | 26 | 1693 | 400 |
| Reverse | 5′-gtctcctgcaaaggaaagagat-3′ | 22 | 1725 | 401 |

TABLE AHB

Probe Name Ag90

| | | | | |
|---|---|---|---|---|
| | | | Start | |
| Primers | Sequences | Length | Position | SEQ ID No |

| | | | | |
|---|---|---|---|---|
| Forward | 5′-ttggcctggactgcttcttc-3′ | 20 | 977 | 402 |
| Probe | TET-5′-tcagcgtttgacaatccaacttacga-3′-TAMRA | 26 | 1693 | 400 |
| Reverse | 5′-gtctcctgcaaaggaaagagat-3′ | 24 | 1029 | 404 |

TABLE AHC

Probe Name Ag 124

| | | | Start | |
|---|---|---|---|---|
| Primers | Sequences | Length | Position | SEQ ID No |
| Forward | 5′-cgcccctacaaccgcat-3′ | 17 | 1666 | 405 |
| Probe | TET-5′-ccatagagtcagcgtttgacaatccaacttacg-3′-TAMRA | 33 | 1685 | 406 |
| Reverse | 5′-ctgcaaaggaaagagatccagtc-3′ | 23 | 1719 | 407 |

TABLE AHD
Panel 1

| | Rel. Exp. (%) | Rel. Exp. (%) |
|---|---|---|
| | Ag124, Run | Ag90, Run |
| Tissue Name | 87587871 | 87586258 |
| Endothelial cells | 0.0 | 0.0 |
| Endothelial cells (treated) | 0.0 | 0.0 |
| Pancreas | 0.1 | 0.1 |
| Pancreatic ca. CAPAN 2 | 0.0 | 0.0 |
| Adrenal gland | 0.0 | 0.0 |
| Thyroid | 0.0 | 0.0 |
| Salivary gland | 0.0 | 0.0 |
| Pituitary gland | 0.0 | 0.0 |
| Brain (fetal) | 25.3 | 37.1 |
| Brain (whole) | 24.0 | 22.5 |
| Brain (amygdala) | 26.2 | 24.8 |
| Brain (cerebellum) | 100.0 | 100.0 |
| Brain (hippocampus) | 21.6 | 29.5 |
| Brain (substantia nigra) | 7.7 | 7.6 |
| Brain (thalamus) | 20.0 | 13.7 |
| Brain (hypothalamus) | 8.4 | 7.7 |
| Spinal cord | 1.8 | 1.4 |
| glio/astro U87-MG | 0.0 | 0.0 |
| glio/astro U-118-MG | 0.0 | 0.0 |
| astrocytoma SW1783 | 0.0 | 0.0 |
| neuro*; met SK-N-AS | 0.0 | 0.4 |
| astrocytoma SF-539 | 0.0 | 0.0 |
| astrocytoma SNB-75 | 0.0 | 0.0 |
| glioma SNB-19 | 2.1 | 1.8 |
| glioma U251 | 0.5 | 0.4 |
| glioma SF-295 | 0.0 | 0.0 |
| Heart | 0.0 | 0.0 |
| Skeletal muscle | 0.0 | 0.0 |
| Bone marrow | 0.0 | 0.0 |
| Thymus | 0.0 | 0.1 |
| Spleen | 0.0 | 0.0 |
| Lymph node | 0.0 | 0.0 |
| Colon (ascending) | 0.0 | 0.1 |
| Stomach | 0.0 | 0.1 |
| Small intestine | 0.5 | 0.3 |
| Colon ca. SW480 | 0.0 | 0.0 |
| Colon ca.* SW620 (SW480 met) | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon ca. HCT-15 | 0.0 | 0.0 |
| Colon ca. HCC-2998 | 0.0 | 0.0 |
| Gastric ca. * (liver met) NCI-N87 | 0.0 | 0.0 |
| Bladder | 0.0 | 0.0 |
| Trachea | 0.0 | 0.0 |
| Kidney | 0.0 | 0.0 |
| Kidney (fetal) | 0.0 | 0.0 |
| Renal ca. 786-0 | 0.0 | 0.0 |
| Renal ca. A498 | 0.0 | 0.0 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.0 | 0.0 |
| Liver (fetal) | 0.0 | 0.0 |
| Liver ca. (hepatoblast) HepG2 | 0.0 | 0.0 |
| Lung | 0.0 | 0.0 |
| Lung (fetal) | 0.0 | 0.0 |
| Lung ca. (small cell) LX-1 | 0.0 | 0.0 |
| Lung ca. (small cell) NCI-H69 | 43.5 | 33.7 |
| Lung ca. (s. cell var.) SHP-77 | 0.0 | 0.0 |
| Lung ca. (large cell) NCI-H460 | 0.0 | 0.0 |
| Lung ca. (non-sm. cell) A549 | 0.0 | 0.0 |
| Lung ca. (non-s. cell) NCI-H23 | 0.0 | 0.0 |
| Lung ca. (non-s. cell) HOP-62 | 0.0 | 0.0 |
| Lung ca. (non-s. cl) NCI-H522 | 0.0 | 0.0 |
| Lung ca. (squam.) SW900 | 0.0 | 0.0 |
| Lung ca. (squam.) NCI-H596 | 26.8 | 20.0 |
| Mammary gland | 0.0 | 0.1 |
| Breast ca.* (pl. ef) MCF-7 | 0.0 | 0.0 |
| Breast ca.* (pl. ef) MDA-MB-231 | 0.0 | 0.0 |
| Breast ca.* (pl. ef) T47D | 0.0 | 0.0 |
| Breast ca. BT-549 | 0.0 | 0.0 |
| Breast ca. MDA-N | 0.0 | 0.0 |
| Ovary | 0.0 | 0.0 |
| Ovarian ca. OVCAR-3 | 0.0 | 0.0 |
| Ovarian ca. OVCAR-4 | 0.0 | 0.0 |
| Ovarian ca. OVCAR-5 | 0.0 | 0.0 |
| Ovarian ca. OVCAR-8 | 0.0 | 0.0 |
| Ovarian ca. IGROV-1 | 0.0 | 0.0 |
| Ovarian ca. (ascites) SK-OV-3 | 0.0 | 0.0 |
| Uterus | 0.0 | 0.0 |
| Placenta | 0.0 | 0.0 |
| Prostate | 0.0 | 0.0 |
| Prostate ca.* (bone met) PC-3 | 0.0 | 0.0 |
| Testis | 1.0 | 1.3 |
| Melanoma Hs688(A).T | 0.0 | 0.0 |
| Melanoma* (met) Hs688(B).T | 0.0 | 0.0 |
| Melanoma UACC-62 | 0.0 | 0.0 |
| Melanoma M14 | 0.0 | 0.0 |
| Melanoma LOX IMVI | 0.0 | 0.0 |
| Melanoma* (met) SK-MEL-5 | 0.0 | 0.0 |
| Melanoma SK-MEL-28 | 0.0 | 0.0 |

TABLE AHE
Panel 1.3D

| | Rel. Exp. (%) |
|---|---|
| | Ag2796, Run |
| Tissue Name | 165527192 |
| Liver adenocarcinoma | 0.0 |
| Pancreas | 0.0 |
| Pancreatic ca. CAPAN 2 | 0.0 |
| Adrenal gland | 0.2 |
| Thyroid | 0.0 |
| Salivary gland | 0.0 |
| Pituitary gland | 10.0 |
| Brain (fetal) | 100.0 |
| Brain (whole) | 65.1 |
| Brain (amygdala) | 76.8 |
| Brain (cerebellum) | 76.8 |
| Brain (hippocampus) | 51.4 |
| Brain (substantia nigra) | 11.7 |
| Brain (thalamus) | 70.2 |
| Cerebral Cortex | 37.9 |
| Spinal cord | 3.5 |
| glio/astro U87-MG | 0.0 |
| glio/astro U-118-MG | 0.1 |
| astrocytoma SW1783 | 0.0 |
| neuro*; met SK-N-AS | 0.4 |
| astrocytoma SF-539 | 0.0 |
| astrocytoma SNB-75 | 0.0 |
| glioma SNB-19 | 3.2 |
| glioma U251 | 4.3 |
| glioma SF-295 | 0.0 |
| Heart (fetal) | 0.0 |
| Heart | 0.0 |
| Skeletal muscle (fetal) | 2.3 |
| Skeletal muscle | 0.0 |
| Bone marrow | 0.0 |
| Thymus | 0.0 |
| Spleen | 0.1 |
| Lymph node | 0.3 |
| Colorectal | 0.0 |
| Stomach | 0.1 |
| Small intestine | 0.6 |
| Colon ca. SW480 | 0.0 |
| Colon ca.* SW620 (SW480 met) | 0.0 |
| Colon ca. HT29 | 0.0 |
| Colon ca. HCT-116 | 3.3 |
| Colon ca. CaCo-2 | 0.0 |
| Colon ca. tissue (ODO3866) | 0.0 |
| Colon ca. HCC-2998 | 0.0 |
| Gastric ca.* (liver met) NCI-N87 | 0.0 |
| Bladder | 0.0 |
| Trachea | 0.0 |
| Kidney | 0.0 |
| Kidney (fetal) | 0.0 |
| Renal ca. 786-0 | 0.0 |
| Renal ca. A498 | 0.4 |
| Renal ca. RXF 393 | 0.0 |
| Renal ca. ACHN | 0.2 |
| Renal ca. UO-31 | 0.0 |
| Renal ca. TK-10 | 0.0 |
| Liver | 0.0 |
| Liver (fetal) | 0.0 |
| Liver ca. (hepatoblast) HepG2 | 0.0 |
| Lung | 0.0 |
| Lung (fetal) | 0.0 |
| Lung ca. (small cell) LX-1 | 0.0 |
| Lung ca. (small cell) NCI-H69 | 23.8 |
| Lung ca. (s. cell var.) SHP-77 | 21.6 |
| Lung ca. (large cell) NCI-H460 | 0.6 |
| Lung ca. (non-sm. cell) A549 | 0.0 |
| Lung ca. (non-s. cell) NCI-H23 | 0.0 |
| Lung ca. (non-s. cell) HOP-62 | 0.0 |
| Lung ca. (non-s. cl) NCI-H522 | 0.0 |
| Lung ca. (squam.) SW 900 | 0.1 |
| Lung ca. (squam.) NCI-H596 | 35.8 |
| Mammary gland | 0.0 |
| Breast ca.* (pl. ef) MCF-7 | 0.0 |
| Breast ca.* (pl. ef) MDA-MB-231 | 0.1 |
| Breast ca.* (pl. ef) T47D | 0.0 |
| Breast ca. BT-549 | 0.1 |
| Breast ca. MDA-N | 0.0 |
| Ovary | 0.0 |
| Ovarian ca. OVCAR-3 | 0.0 |
| Ovarian ca. OVCAR-4 | 0.0 |
| Ovarian ca. OVCAR-5 | 0.1 |
| Ovarian ca. OVCAR-8 | 0.0 |
| Ovarian ca. IGROV-1 | 0.0 |
| Ovarian ca.* (ascites) SK-OV-3 | 0.0 |
| Uterus | 0.1 |
| Placenta | 1.9 |
| Prostate | 0.0 |
| Prostate ca.* (bone met) PC-3 | 0.0 |
| Testis | 2.0 |
| Melanoma Hs688(A).T | 0.0 |
| Melanoma* (met) Hs688(B).T | 0.0 |
| Melanoma UACC-62 | 0.0 |
| Melanoma M14 | 0.0 |
| Melanoma LOX IMVI | 0.0 |
| Melanoma* (met) SK-MEL-5 | 0.0 |
| Adipose | 0.0 |

TABLE AHF
Panel 2D

| | Rel. Exp. (%) |
|---|---|
| | Ag2796, Run |
| Tissue Name | 162570140 |
| Normal Colon | 26.6 |
| CC Well to Mod Diff (OD03866) | 0.0 |
| CC Margin (ODO3866) | 4.2 |
| CC Gr.2 rectosigmoid (ODO3868) | 0.0 |
| CC Margin (ODO3868) | 0.7 |
| CC Mod Diff (ODO3920) | 0.0 |
| CC Margin (ODO3920) | 13.1 |
| CC Gr.2 ascend colon (ODO3921) | 4.8 |
| CC Margin (ODO3921) | 5.1 |
| CC from Partial Hepatectomy (ODO4309) Mets | 8.5 |
| Liver Margin (ODO4309) | 0.0 |
| Colon mets to lung (OD04451-01) | 0.0 |
| Lung Margin (OD04451 -02) | 1.1 |
| Normal Prostate 6546-1 | 17.6 |
| Prostate Cancer (OD04410) | 12.3 |
| Prostate Margin (OD04410) | 6.2 |
| Prostate Cancer (OD04720-01) | 11.5 |
| Prostate Margin (OD04720-02) | 18.3 |
| Normal Lung 061010 | 2.0 |
| Lung Met to Muscle (ODO4286) | 0.0 |
| Muscle Margin (ODO4286) | 0.7 |
| Lung Malignant Cancer (OD03126) | 1.0 |
| Lung Margin (OD03126) | 2.1 |
| Lung Cancer (OD04404) | 1.8 |
| Lung Margin (OD04404) | 2.1 |
| Lung Cancer (OD04565) | 5.6 |
| Lung Margin (OD04565) | 0.0 |
| Lung Cancer (OD04237-01) | 48.0 |
| Lung Margin (OD04237-02) | 0.0 |
| Ocular Mel Met to Liver (ODO4310) | 2.8 |
| Liver Margin (ODO4310) | 0.0 |
| Melanoma Mets to Lung (OD04321) | 1.3 |
| Lung Margin (OD04321) | 21.6 |
| Normal Kidney | 6.9 |
| Kidney Ca, Nuclear grade 2 (OD04338) | 6.4 |
| Kidney, Margin (OD04338) | 2.0 |
| Kidney Ca Nuclear grade 1/2 (OD04339) | 0.9 |
| Kidney Margin (OD04339) | 3.0 |
| Kidney Ca, Clear cell type (OD04340) | 3.0 |
| Kidney Margin (OD04340) | 1.3 |
| Kidney Ca, Nuclear grade 3 (OD04348) | 0.7 |
| Kidney Margin (OD04348) | 2.5 |
| Kidney Cancer (OD04622-01) | 0.8 |
| Kidney Margin (OD04622-03) | 0.0 |
| Kidney Cancer (OD04450-01) | 3.9 |
| Kidney Margin (OD04450-03) | 2.2 |
| Kidney Cancer 8120607 | 0.8 |
| Kidney Margin 8120608 | 0.0 |
| Kidney Cancer 8120613 | 0.0 |
| Kidney Margin 8120614 | 1.7 |
| Kidney Cancer 9010320 | 3.5 |
| Kidney Margin 9010321 | 3.7 |
| Normal Uterus | 3.7 |
| Uterus Cancer 064011 | 6.0 |
| Normal Thyroid | 0.0 |
| Thyroid Cancer 064010 | 0.0 |
| Thyroid Cancer A302152 | 3.5 |
| Thyroid Margin A302153 | 0.0 |
| Normal Breast | 4.5 |
| Breast Cancer (OD04566) | 1.8 |
| Breast Cancer (OD04590-01) | 3.0 |
| Breast Cancer Mets (OD04590-03) | 6.3 |
| Breast Cancer Metastasis (OD04655-05) | 9.2 |
| Breast Cancer 064006 | 2.3 |
| Breast Cancer 1024 | 4.3 |
| Breast Cancer 9100266 | 4.1 |
| Breast Margin 9100265 | 0.0 |
| Breast Cancer A209073 | 3.0 |
| Breast Margin A209073 | 0.7 |
| Normal Liver | 0.0 |
| Liver Cancer 064003 | 0.0 |
| Liver Cancer 1025 | 3.7 |
| Liver Cancer 1026 | 95.9 |
| Liver Cancer 6004-T | 0.0 |
| Liver Tissue 6004-N | 7.2 |
| Liver Cancer 6005-T | 100.0 |
| Liver Tissue 6005-N | 0.0 |
| Normal Bladder | 8.8 |
| Bladder Cancer 1023 | 0.0 |
| Bladder Cancer A302173 | 1.5 |
| Bladder Cancer (OD04718-01) | 1.8 |
| Bladder Normal Adjacent (OD04718-03) | 2.8 |
| Normal Ovary | 4.9 |
| Ovarian Cancer 064008 | 3.6 |
| Ovarian Cancer (OD04768-07) | 2.5 |
| Ovary Margin (OD04768-08) | 0.0 |
| Normal Stomach | 3.7 |
| Gastric Cancer 9060358 | 0.6 |
| Stomach Margin 9060359 | 4.8 |
| Gastric Cancer 9060395 | 1.8 |
| Stomach Margin 9060394 | 1.0 |
| Gastric Cancer 9060397 | 0.0 |
| Stomach Margin 9060396 | 1.0 |
| Gastric Cancer 064005 | 5.3 |

TABLE AHG
Panel 4D

| | Rel. Exp. (%) |
|---|---|
| | Ag2796, Run |
| Tissue Name | 162292586 |
| Secondary Th1 act | 4.3 |
| Secondary Th2 act | 8.9 |
| Secondary Tr1 act | 0.0 |
| Secondary Th1 rest | 0.0 |
| Secondary Th2 rest | 0.0 |
| Secondary Tr1 rest | 4.7 |
| Primary Th1 act | 6.2 |
| Primary Th2 act | 0.0 |
| Primary Tr1 act | 4.0 |
| Primary Th1 rest | 4.1 |
| Primary Th2 rest | 0.0 |
| Primary Tr1 rest | 0.0 |
| CD45RA CD4 lymphocyte act | 8.2 |
| CD45RO CD4 lymphocyte act | 6.0 |
| CD8 lymphocyte act | 8.4 |
| Secondary CD8 lymphocyte rest | 0.0 |
| Secondary CD8 lymphocyte act | 9.5 |
| CD4 lymphocyte none | 0.0 |
| 2ry Th1/Th2/Tr1_anti-CD95 CH11 | 14.5 |
| LAK cells rest | 0.0 |
| LAK cells IL-2 | 16.8 |
| LAK cells IL-2 + IL-12 | 3.8 |
| LAK cells IL-2 + IFN gamma | 3.9 |
| LAK cells IL-2 + IL-18 | 21.8 |
| LAK cells PMA/ionomycin | 0.0 |
| NK Cells IL-2 rest | 15.4 |
| Two Way MLR 3 day | 18.8 |
| Two Way MLR 5 day | 7.9 |
| Two Way MLR 7 day | 3.1 |
| PBMC rest | 6.1 |
| PBMC PWM | 20.4 |
| PBMC PHA-L | 6.5 |
| Ramos (B cell) none | 3.3 |
| Ramos (B cell) ionomycin | 23.0 |
| B lymphocytes PWM | 35.6 |
| B lymphocytes CD40L and IL-4 | 70.7 |
| EOL-1 dbcAMP | 7.4 |
| EOL-1 dbcAMP PMA/ionomycin | 51.1 |
| Dendritic cells none | 10.6 |
| Dendritic cells LPS | 20.9 |
| Dendritic cells anti-CD40 | 9.8 |
| Monocytes rest | 9.3 |
| Monocytes LPS | 9.6 |
| Macrophages rest | 4.6 |
| Macrophages LPS | 5.4 |
| HUVEC none | 11.3 |
| HUVEC starved | 18.4 |
| HUVEC IL-1beta | 0.0 |
| HUVEC IFN gamma | 8.1 |
| HUVEC TNF alpha + IFN gamma | 4.7 |
| HUVEC TNF alpha + IL4 | 0.0 |
| HUVEC IL-11 | 4.2 |
| Lung Microvascular EC none | 3.7 |
| Lung Microvascular EC TNFalpha + IL-1beta | 3.7 |
| Microvascular Dermal EC none | 9.9 |
| Microsvasular Dermal EC TNFalpha + IL-1beta | 4.2 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 34.9 |
| Astrocytes TNFalpha + IL-1beta | 42.3 |
| KU-8l2 (Basophil) rest | 0.0 |
| KU-8l2 (Basophil) PMA/ionomycin | 14.2 |
| CCD1106 (Keratinocytes) none | 0.0 |
| CCD1106 (Keratinocytes) TNFalpha + IL-1beta | 0.0 |
| Liver cirrhosis | 3.8 |
| Lupus kidney | 0.0 |
| NCI-H292 none | 30.6 |
| NCI-H292 IL-4 | 15.3 |
| NCI-H292 IL-9 | 30.1 |
| NCI-H292 IL-13 | 18.2 |
| NCI-H292 IFN gamma | 8.3 |
| HPAEC none | 0.0 |
| HPAEC TNF alpha + IL-1 beta | 0.0 |
| Lung fibroblast none | 11.2 |
| Lung fibroblast TNF alpha + IL-1 beta | 0.0 |
| Lung fibroblast IL-4 | 7.5 |
| Lung fibroblast IL-9 | 0.0 |
| Lung fibroblast IL-13 | 4.2 |
| Lung fibroblast IFN gamma | 9.5 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 8.9 |
| IBD Colitis 2 | 0.0 |
| IBD Crohn's | 0.0 |
| Colon | 100.0 |
| Lung | 17.2 |
| Thymus | 20.3 |
| Kidney | 33.0 |

Panel 1 Summary: Ag90/Ag2796 Two experiments with different probe and primer sets are in excellent agreement. Highest expression of this gene is detected in brain cerebellum (CT=25-26). High levels of expression of this gene is mainly seen in all the regions of brain including amygdala, hippocampus, substantia nigra, thalamus, cerebellum, cerebral cortex, and spinal cord. In addition, moderate levels of expression of this gene is also seen in two lung cancer cell lines and a glioma cell line. See panel 10.3D for further discussion of this gene.

Panel 1.3D Summary: Ag2796 Highest expression of this gene is detected in fetal brain (CT=28.7). Moderate levels of expression of this gene is mainly seen in all the regions of brain including amygdala, hippocampus, substantia nigra, thalamus, cerebellum, cerebral cortex, and spinal cord. Therefore, therapeutic modulation of this gene product may be useful in the treatment of central nervous system disorders such as Alzheiiner's disease, Parkinson's disease, epilepsy, multiple sclerosis, schizophrenia and depression.

This gene codes for a homolog of mouse seizure related protein, SEZ-6. Mouse SEZ-6 was first isolated from cerebrum cortex-derived cells treated with pentylentetrazole (PTZ), one of the convulsant drugs (Shimizu-Nishikawa et al., 1995, Brain Res Mol Brain Res 28(2):201-10, PMID: 7723619). Thus, SEZ-6 protein encoded by this gene may also play a role in brain seizure.

In addition, moderate to low levels of expression of this gene is also seen in three lung cancer cell lines, two of the glioma cell lines and a colon cancer cell line. Therefore, expression of this gene may be used as diagnostic marker to detect lung, colon and brain cancers. Furthermore, modulation of this gene or its protein product through the use of antibody or protein therapeutics, may be useful in the treatment of lung, colon and brain cancers.

Significant expression is also detected in fetal skeletal muscle. This gene is expressed at much higher levels in fetal (CT=34.1) when compared to adult skeletal muscle (CT=40). This observation suggests that expression of this gene can be used to distinguish fetal from adult skeletal muscle. In addition, the relative overexpression of this gene in fetal skeletal muscle suggests that the protein product may enhance muscular growth or development in the fetus and thus may also act in a regenerative capacity in the adult. Therefore, therapeutic modulation of the SEZ-6 encoded by this gene could be useful in treatment of muscle related diseases. More specifically, treatment of weak or dystrophic muscle with the protein encoded by this gene could restore muscle mass or function.

Panel 2D Summary: Ag2796 Highest expression of this gene is detected in two liver cancer samples (CTs=32.3). In addition, low levels of expression of this gene is also seen in a lung cancer sample. Expression of this gene is higher in lung and liver cancer as compared to corresponding adjacent normal tissue (CTs=40). Thus, expression of this gene may be used to distinguish between normal and cancer samples and as diagnostic marker to detect lung and liver cancer. In addition, therapeutic modulation of this gene through the use of antibodies may be useful in the treatment of these cancers.

Panel 4D Summary: Ag2796 Low but significant expression of this gene is detected exclusively in colon (CT=34.8). Therefore, expression of this gene may be used to distinguish colon from the other tissues on this panel. Furthermore, expression of this gene is decreased in colon samples from patients with inflammatory bowel disease, colitis and Crohn's disease relative to normal colon. Therefore, therapeutic modulation of the activity of the SEZ-6 protein encoded by this gene may be useful in the treatment of inflammatory bowel disease.

AI. CG55698-02: Colipase Precursor Protein-Like Protein.

Expression of gene CG55698-02 was assessed using the primer-probe set Ag7086, described in Table AIA. Results of the RTQ-PCR runs are shown in Table AIB. Note that CG55698-02 represents a full-length physical clone.

TABLE AIA

Probe Name Ag7086

| | | | | |
|---|---|---|---|---|
| | | | Start | |
| Primers | Sequences | Length | Position | SEQ ID No |

| | | | | |
|---|---|---|---|---|
| Forward | 5′-cattatcaacctgacgctctatg-3′ | 23 | 88 | 408 |
| Probe | TET-5′-ccacgctcacagggacacttgtagta-3′-TAMRA | 26 | 116 | 409 |
| Reverse | 5′-atggtcttgtctccctcaca-3′ | 20 | 149 | 410 |

TABLE AIB
General_screening_panel_v1.6

| | Rel. Exp. (%) |
|---|---|
| | Ag7086, Run |
| Tissue Name | 296433065 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.4 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 12.1 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 100.0 |

CNS_neurodegeneration v1.0 Summary: Ag7086 Expression of this gene is low/undetectable (CTs>35) across all of the samples on this panel (data not shown).

General_screening_panel_v1.6 Summary: Ag7086 Highest expression of this gene is seen in pancreas (CT=22.7). Therefore, expression of this gene may be used to distinguish pancrease from other samples in this panel. This gene codes for a deletion variant of colipase. Pancreatic colipase is a 12-kD polypeptide cofactor for pancreatic lipase, an enzyme essential for the absorption of dietary long-chain triglyceride fatty acids. Colipase is thought to anchor lipase noncovalently to the surface of lipid micelles, counteracting the destabilizing influence of intestinal bile salts (OMIM 120105). Therefore, therapeutic modulation of expression of this gene or colipase encoded by this gene may be useful in the treatment of dietary fat related disorders including pancreatic insufficiency and fat malabsorption.

AJ. CG55832-02 and CG55832-03: Tenascin-C Precursor Protein-Like Protein.

Expression of gene CG55832-02 and CG55832-03 was assessed using the primer-probe set Ag4681, described in Table AJA. Results of the RTQ-PCR runs are shown in Tables AJB, AJC and AJD.

TABLE AJA

Probe Name Ag4681

| | | | Start | |
|---|---|---|---|---|
| Primers | Sequences | Length | Position | SEQ ID No |
| Forward | 5′-tgttccaaagagccaacaag-3′ | 20 | 2868 | 411 |
| Probe | TET-5′-ccaaaaccacactcacaggtctgagg-3′-TAMRA | 26 | 2894 | 412 |
| Reverse | 5′-agcagaaactccaatcccatat-3′ | 22 | 2931 | 413 |

TABLE AJB
General_screening_panel_v1.4

| | Rel. Exp. (%) |
|---|---|
| | Ag4681, Run |
| Tissue Name | 222811927 |
| Adipose | 0.9 |
| Melanoma* Hs688(A).T | 18.2 |
| Melanoma* Hs688(B).T | 7.3 |
| Melanoma* M14 | 10.8 |
| Melanoma* LOXIMVI | 7.7 |
| Melanoma* SK-MEL-5 | 3.8 |
| Squamous cell carcinoma SCC-4 | 1.2 |
| Testis Pool | 1.0 |
| Prostate ca.* (bone met) PC-3 | 7.7 |
| Prostate Pool | 1.7 |
| Placenta | 0.1 |
| Uterus Pool | 2.5 |
| Ovarian ca. OVCAR-3 | 3.2 |
| Ovarian ca. SK-OV-3 | 0.2 |
| Ovarian ca. OVCAR-4 | 0.5 |
| Ovarian ca. OVCAR-5 | 0.2 |
| Ovarian ca. IGROV-1 | 13.9 |
| Ovarian ca. OVCAR-8 | 4.6 |
| Ovary | 0.2 |
| Breast ca. MCF-7 | 0.0 |
| Breast ca. MDA-MB-231 | 2.6 |
| Breast ca. BT 549 | 2.4 |
| Breast ca. T47D | 0.4 |
| Breast ca. MDA-N | 3.7 |
| Breast Pool | 4.5 |
| Trachea | 3.1 |
| Lung | 0.1 |
| Fetal Lung | 8.7 |
| Lung ca. NCI-N417 | 0.2 |
| Lung ca. LX-1 | 3.6 |
| Lung ca. NCI-H146 | 0.0 |
| Lung ca. SHP-77 | 1.0 |
| Lung ca. A549 | 0.0 |
| Lung ca. NCI-H526 | 0.0 |
| Lung ca. NCI-H23 | 0.1 |
| Lung ca. NCI-H460 | 0.0 |
| Lung ca. HOP-62 | 11.8 |
| Lung ca. NCI-H522 | 0.0 |
| Liver | 0.1 |
| Fetal Liver | 0.2 |
| Liver ca. HepG2 | 0.0 |
| Kidney Pool | 11.3 |
| Fetal Kidney | 7.1 |
| Renal ca. 786-0 | 2.2 |
| Renal ca. A498 | 0.3 |
| Renal ca. ACHN | 0.5 |
| Renal ca. UO-31 | 1.3 |
| Renal ca. TK-10 | 0.4 |
| Bladder | 1.3 |
| Gastric ca. (liver met.) NCI-N87 | 1.3 |
| Gastric ca. KATO III | 0.3 |
| Colon ca. SW-948 | 0.0 |
| Colon ca. SW480 | 0.2 |
| Colon ca.* (SW480 met) SW620 | 6.7 |
| Colon ca. HT29 | 0.0 |
| Colon ca. HCT-116 | 0.0 |
| Colon ca. CaCo-2 | 0.3 |
| Colon cancer tissue | 3.7 |
| Colon ca. SW1116 | 0.1 |
| Colon ca. Colo-205 | 0.3 |
| Colon ca. SW-48 | 0.0 |
| Colon Pool | 5.9 |
| Small Intestine Pool | 3.4 |
| Stomach Pool | 2.9 |
| Bone Marrow Pool | 2.9 |
| Fetal Heart | 0.1 |
| Heart Pool | 3.7 |
| Lymph Node Pool | 7.9 |
| Fetal Skeletal Muscle | 0.4 |
| Skeletal Muscle Pool | 1.0 |
| Spleen Pool | 2.0 |
| Thymus Pool | 2.5 |
| CNS cancer (glio/astro) U87-MG | 29.1 |
| CNS cancer (glio/astro) U-118-MG | 100.0 |
| CNS cancer (neuro; met) SK-N-AS | 0.9 |
| CNS cancer (astro) SF-539 | 3.6 |
| CNS cancer (astro) SNB-75 | 37.4 |
| CNS cancer (glio) SNB-19 | 13.1 |
| CNS cancer (glio) SF-295 | 8.2 |
| Brain (Amygdala) Pool | 0.4 |
| Brain (cerebellum) | 0.3 |
| Brain (fetal) | 3.4 |
| Brain (Hippocampus) Pool | 0.4 |
| Cerebral Cortex Pool | 0.4 |
| Brain (Substantia nigra) Pool | 0.5 |
| Brain (Thalamus) Pool | 0.7 |
| Brain (whole) | 0.9 |
| Spinal Cord Pool | 1.0 |
| Adrenal Gland | 0.4 |
| Pituitary gland Pool | 0.2 |
| Salivary Gland | 0.3 |
| Thyroid (female) | 0.1 |
| Pancreatic ca. CAPAN2 | 0.4 |
| Pancreas Pool | 3.5 |

TABLE AJC
Oncology_cell_line_screening_panel_v3.1

| | Rel. Exp. (%) |
|---|---|
| | Ag4681, Run |
| Tissue Name | 224056814 |
| Daoy Medulloblastoma/Cerebellum | 8.6 |
| TE671 Medulloblastom/Cerebellum | 9.2 |
| D283 Med Medulloblastoma/Cerebellum | 0.1 |
| PFSK-1 Primitive | 4.4 |
| Neuroectodermal/Cerebellum | |
| XF-498_CNS | 100.0 |
| SNB-78_CNS/glioma | 66.4 |
| SF-268_CNS/glioblastoma | 2.0 |
| T98G_Glioblastoma | 5.0 |
| SK-N-SH_Neuroblastoma (metastasis) | 78.5 |
| SF-295_CNS/glioblastoma | 6.4 |
| Cerebellum | 0.2 |
| Cerebellum | 0.4 |
| NCI-H292_Mucoepidermoid lung ca. | 1.6 |
| DMS-114_Small cell lung cancer | 0.4 |
| DMS-79_Small cell lung | 0.2 |
| cancer/neuroendocrine | |
| NCI-H146_Small cell lung | 0.0 |
| cancer/neuroendocrine | |
| NCI-H526_Small cell lung | 0.1 |
| cancer/neuroendocrine | |
| NCI-N417_Small cell lung | 1.8 |
| cancer/neuroendocrine | |
| NCI-H82_Small cell lung | 0.0 |
| cancer/neuroendocrine | |
| NCI-H157_Squamous cell lung | 5.6 |
| cancer (metastasis) | |
| NCI-H1155_Large cell lung | 0.0 |
| cancer/neuroendocrine | |
| NCI-H1299_Large cell lung | 0.3 |
| cancer/neuroendocrine | |
| NCI-H727_Lung carcinoid | 0.2 |
| NCI-UMC-11_Lung carcinoid | 0.0 |
| LX-1_Small cell lung cancer | 9.0 |
| Colo-205_Colon cancer | 1.3 |
| KM12_Colon cancer | 3.3 |
| KM20L2_Colon cancer | 0.2 |
| NCI-H716_Colon cancer | 0.0 |
| SW-48_Colon adenocarcinoma | 0.0 |
| SW1116_Colon adenocarcinoma | 0.4 |
| LS 174T_Colon adenocarcinoma | 2.4 |
| SW-948_Colon adenocarcinoma | 0.1 |
| SW-480_Colon adenocarcinoma | 0.1 |
| NCI-SNU-5_Gastric ca. | 0.2 |
| KATO III_Stomach | 0.2 |
| NCI-SNU-16_Gastric ca. | 25.7 |
| NCI-SNU-1_Gastric ca. | 0.0 |
| RF-1_Gastric adenocarcinoma | 0.0 |
| RF-48_Gastric adenocarcinoma | 0.0 |
| MKN-45_Gastric ca. | 0.3 |
| NCI-N87_Gastric ca. | 0.2 |
| OVCAR-5_Ovarian ca. | 0.3 |
| RL95-2_Uterine carcinoma | 0.0 |
| HelaS3_Cervicaladenocarcinoma | 0.0 |
| Ca Ski_Cervical epidermoid | 18.7 |
| carcinoma (metastasis) | |
| ES-2 Ovarian clear cell carcinoma | 6.0 |
| Ramos/6 h stim_Stimulated with | 0.0 |
| PMA/ionomycin 6 h | |
| Ramos/14 h stim_Stimulated with | 0.0 |
| PMA/ionomycin 14 h | |
| MEG-01_Chronic myelogenous leukemia | 0.0 |
| (megokaryoblast) | |
| Raji_Burkitt's lymphoma | 0.0 |
| Daudi_Burkitt's lymphoma | 0.0 |
| U266_B-cell plasmacytoma/myeloma | 0.0 |
| CA46_Burkitt's lymphoma | 0.0 |
| RL_non-Hodgkin's B-cell lymphoma | 0.0 |
| JM1_pre-B-cell lymphoma/leukemia | 0.0 |
| Jurkat_T cell leukemia | 0.0 |
| TF-1_Erythroleukemia | 0.0 |
| HUT 78_T-cell lymphoma | 0.0 |
| U937_Histiocytic lymphoma | 0.0 |
| KU-812_Myelogenous leukemia | 0.0 |
| 769-P_Clear cell renal ca. | 0.0 |
| Caki-2_Clear cell renal ca. | 0.7 |
| SW 839_Clear cell renal ca. | 4.1 |
| G401_Wilms' tumor | 0.0 |
| Hs766T_Pancreatic ca. (LN metastasis) | 0.0 |
| CAPAN-1_Pancreatic adenocarcinoma | 0.1 |
| (liver metastasis) | |
| SU86.86_Pancreatic carcinoma (liver | 4.3 |
| metastasis) | |
| BxPC-3_Pancreatic adenocarcinoma | 0.0 |
| HPAC_Pancreatic adenocarcinoma | 0.5 |
| MIA PaCa-2_Pancreatic ca. | 0.1 |
| CFPAC-1_Pancreatic ductal adenocarcinoma | 10.3 |
| PANC-1_Pancreatic epithelioid ductal ca. | 0.9 |
| T24_Bladder ca. (transitional cell) | 0.1 |
| 5637_Bladder ca. | 13.8 |
| HT-1197_Bladder ca. | 0.1 |
| HJM-UC-3_Bladder ca. (transitional cell) | 2.9 |
| A204_Rhabdomyosarcoma | 61.1 |
| HT-1080_Fibrosarcoma | 1.3 |
| MG-63_Osteosarcoma (bone) | 2.2 |
| SK-LMS-1_Leiomyosarcoma (vulva) | 28.1 |
| SJRH30_Rhabdomyosarcoma (met to bone marrow) | 23.0 |
| A431_Epidermoid ca. | 0.1 |
| WM266-4_Melanoma | 51.1 |
| DU 145_Prostate | 4.5 |
| MDA-MB-468_Breast adenocarcinoma | 0.0 |
| SSC-4_Tongue | 3.3 |
| SSC-9_Tongue | 15.5 |
| SSC-15_Tongue | 9.9 |
| CAL 27_Squamous cell ca. of tongue | 31.9 |

TABLE AJD
Panel 4.1D

| | Rel. Exp. (%) |
|---|---|
| | Ag4681, Run |
| Tissue Name | 268722514 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 14.4 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.1 |
| Lak cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.4 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.1 |
| EOL-1 dbcAMP | 0.0 |
| EOL-1 dbcAMP PMA/ionomycin | 0.0 |
| Dendritic cells none | 0.6 |
| Dendritic cells LPS | 0.6 |
| Dendritic cells anti-CD40 | 0.2 |
| Monocytes rest | 0.0 |
| Monocytes LPS | 0.1 |
| Macrophages rest | 0.1 |
| Macrophages LPS | 0.2 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 8.5 |
| Small airway epithelium none | 3.1 |
| Small airway epithelium TNFalpha + IL-1beta | 10.2 |
| Coronery artery SMC rest | 1.4 |
| Coronery artery SMC TNFalpha + IL-1beta | 3.5 |
| Astrocytes rest | 9.6 |
| Astrocytes TNFalpha + IL-1beta | 7.7 |
| KU-812 (Basophil) rest | 0.0 |
| KU-812 (Basophil) PMA/ionomycin | 0.0 |
| CCD1106 (Keratinocytes) none | 15.1 |
| CCD1106 (Keratinocytes) TNFalpha + IL-1beta | 22.4 |
| Liver cirrhosis | 0.6 |
| NCI-H292 none | 0.1 |
| NCI-H292 IL-4 | 1.1 |
| NCI-H292 IL-9 | 0.1 |
| NCI-H292 IL-13 | 2.2 |
| NCI-H292 IFN gamma | 0.1 |
| HPAEC none | 0.0 |
| HPAEC TNF alpha + IL-1 beta | 0.0 |
| Lung fibroblast none | 46.0 |
| Lung fibroblast TNF alpha + IL-1 beta | 19.8 |
| Lung fibroblast IL-4 | 74.7 |
| Lung fibroblast IL-9 | 75.3 |
| Lung fibroblast IL-13 | 44.4 |
| Lung fibroblast IFN gamma | 53.2 |
| Dermal fibroblast CCD1070 rest | 27.7 |
| Dermal fibroblast CCD1070 TNF alpha | 26.4 |
| Dermal fibroblast CCD1070 IL-1 beta | 31.9 |
| Dermal fibroblast IFN gamma | 12.8 |
| Dermal fibroblast IL-4 | 100.0 |
| Dermal Fibroblasts rest | 4.6 |
| Neutrophils TNFa + LPS | 0.0 |
| Neutrophils rest | 0.0 |
| Colon | 0.5 |
| Lung | 0.4 |
| Thymus | 0.2 |
| Kidney | 1.2 |

General_screening_panel v1.4 Summary: Ag4681 Highest expression of this gene is seen in a brain cancer cell line (CT=20.3). Prominent expression of this gene is also seen in a cluster of samples derived from brain cancer cell lines. High levels of expression are also seen in cell lines from colon, renal, ovarian, lung, breast, prostate, and melanoma cancers. This gene encodes a homolog of tenascin-C, an extracellular matrix protein that appears at active sites of tissue remodelling during cancer invasion. Tenascin has been shown to be highly expressed around tumours, including invasive breast carcinomas and may be expressed by these invasive carcinomas (Adams M. Cancer Res 2002 June 1;62(11):3289-97). Zagzag et. al has suggested a potential role for tenascin-C in pathological angiogenesis (Cancer Res May 1, 2002;62(9):2660-8). Thus, expression of this gene could be used to differentiate between these cell lines and other samples on this panel, and as a marker of brain cancer. Based on the homology of this gene to tenascin-C and the expression in brain cancer cell lines, therapeutic modulation of the expression or function of this protein may be useful in the treatment of colon, brain, renal, ovarian, lung, breast, prostate, and melanoma cancers.

Oncology_cell_line_screening_panel_v3.1 Summary: Ag4681 Highest expression of this gene is seen in a brain cancer cell line (CT=23.7), consistent with expression in panel 1.4. In addition, high levels of expression are seen in other cell lines on this panel, including samples from gastric and lung cancers. See Panel 1.4 for discussion of this gene in cancer.

Panel 4.1D Summary: Ag4681 Highest expression of this gene is seen in IL-4 treated dermal fibroblasts (CT=22.72). High levels of expression of this gene are seen in treated and untreated lung and dermal fibroblasts, keratinocytes, astrocytes, and bronchial and small airway epithelium. Moderate to low levels of expression of this gene is also seen in naive T cells, resting and activated dendritic cells and activated B lymphocytes. Expression of this gene in dendritic cells suggests a role for this gene in antigen presentation. This gene has homology to tenascin-C, an extracellular matrix glycoprotein that is expressed during inflammatory and fibrotic disorders, and specifically, is deposited in increased amounts in the asthmatic airway (Johnson PR. Clin Exp Pharmacol Physiol March 2001;28(3):233-6). The preferential expression of this gene in cells derived from the lung and skin suggests that this gene product may be involved in normal conditions as well as pathological and inflammatory lung and skin disorders that include chronic obstructive pulmonary disease, asthma, allergy, psoriasis and emphysema.

AK. CG56054-02: Integrin Alpha 7-Like Protein.

Expression of gene CG56054-02 was assessed using the primer-probe sets Ag4983, Ag6442, Ag6424, Ag6428, Ag6429, Ag6430, Ag6431, Ag6439, Ag6413 and Ag6964, described in Tables AKA, AKB, AKC, AKD, AKE, AKF, AKG, AKH, AKI and AKJ. Results of the RTQ-PCR runs are shown in Tables AKK, AKL, AKM, AKN, AKO and AKP.

TABLE AKA

Probe Name Ag4983

| | | | | |
|---|---|---|---|---|
| | | | Start | |
| Primers | Sequences | Length | Position | SEQ ID No |

| | | | | |
|---|---|---|---|---|
| Forward | 5′-ccaggtcaccttctacctcatc-3′ | 22 | 2435 | 414 |
| Probe | TET-5′-cttagcacctccgggatcagcatt-3′-TAMRA | 24 | 2457 | 415 |
| Reverse | 5′-aacagcagctctacctccagtt-3′ | 22 | 2491 | 416 |

TABLE AKB

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3′ | 22 | 2874 | 417 |
| Probe | TET-5′-cacctgagcagcaggagcct-3′-TAMRA | 21 | 2913 | 418 |
| Reverse | 5′-gcgcagtccagggtg-3′ | 15 | 2999 | 419 |

TABLE AKC

Probe Name Ag6424

| | | | | |
|---|---|---|---|---|
| Primers | Sequences | Length | Start Position | SEQ ID No |

| | | | | |
|---|---|---|---|---|
| Forward | 5′-ttgggttctgccagca-3′ | 16 | 742 | 420 |
| Probe | TET-5′-cacagcctgccgccttctccc-3′-TAMRA | 20 | 761 | 421 |
| Reverse | 5′-aaaagcaaccccttccaa-3′ | 18 | 824 | 422 |

TABLE AKD

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1394 | 423 |
| Probe | TET-5′-ccttcacaggtgctggagggc-3′-TAMRA | 21 | 1434 | 424 |
| Reverse | 5′-agggagtagccgaagctct-3′ | 19 | 1471 | 425 |

TABLE AKE

Probe Name Ag6429

| | | | | |
|---|---|---|---|---|
| Primers | Sequences | Length | Start Position | SEQ ID No |

| | | | | |
|---|---|---|---|---|
| Forward | 5′-ccgtgccccagtaccat-3′ | 17 | 3382 | 426 |
| Probe | TET-5′-cgggcaccatcctgaggaacaac-3′-TAMRA | 23 | 3448 | 427 |
| Reverse | 5′-gggcccagccaggat-3′ | 15 | 3484 | 428 |

TABLE AKF

Probe Name Ag6430

| | | | Start | |
|---|---|---|---|---|
| Primers | Sequences | Length | Position | SEQ ID No |
| Forward | 5′-gtgaccaacattgatagctcaga-3′ | 23 | 843 | 429 |
| Probe | TET-5′-ccccgaccagctggtgtataaaactttg-3′-TAMRA | 28 | 866 | 430 |
| Reverse | 5′-gggagccggtcagca-3′ | 15 | 899 | 431 |

TABLE AKG

Probe Name Ag6431

| | | | Start | |
|---|---|---|---|---|
| Primers | Sequences | Length | Position | SEQ ID No |
| Forward | 5′-aaacatcaccctggactgc-3′ | 19 | 2993 | 432 |
| Probe | TET-5′-tggtgttcagctgcccactctacag-3′TAMRA | 25 | 3034 | 433 |
| Reverse | 5′-ccgcgcggtcaaa-3′ | 13 | 3060 | 434 |

TABLE AKH

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 3250 | 435 |
| Probe | TET-5′-ccctggtgggtcatcctcctg-3′-TAMRA | 21 | 3270 | 436 |
| Reverse | 5′-gaagaatcccatcttccacag-3′ | 21 | 3336 | 437 |

TABLE AKI

Probe Name Ag6413

| | | | | |
|---|---|---|---|---|
| Primers | Sequences | Length | Start Position | SEQ ID No |

| | | | | |
|---|---|---|---|---|
| Forward | 5′-ggtgaagacaagatctgccag-3′ | 21 | 2073 | 438 |
| Probe | TET-5′-tgtacccgggtcagcgacacg-3′-TAMRA | 21 | 2124 | 439 |
| Reverse | 5′-gctgttgttccatccacatc-3′ | 20 | 2166 | 440 |

TABLE AKJ

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatgca-3′ | 15 | 3079 | 441 |
| Probe | TET-5′-actctacagctttgaccgcgcgg-3′-TAMRA | 23 | 3050 | 442 |
| Reverse | 5′-gccaactgtgtggtgttca-3′ | 19 | 3024 | 1443 |

TABLE AKK
CNS_neurodegeneration_v1.0

| | Rel. | Rel. | Rel. | Rel. | Rel. | Rel. | Rel. |
|---|---|---|---|---|---|---|---|
| | Exp. (%) | Exp. (%) | Exp. (%) | Exp. (%) | Exp. (%) | Exp. (%) | Exp. (%) |
| | Ag4983, | Ag6413, | Ag6428, | Ag6430, | Ag6431, | Ag6439, | Ag6442, |
| | Run | Run | Run | Run | Run | Run | Run |
| Tissue Name | 218649223 | 269253983 | 266937081 | 266937085 | 268030722 | 269254002 | 264979298 |
| AD 1 Hippo | 23.7 | 24.8 | 18.0 | 20.0 | 18.8 | 21.6 | 19.2 |
| AD 2 Hippo | 41.2 | 52.9 | 32.3 | 48.0 | 28.7 | 28.9 | 49.7 |
| AD 3 Hippo | 8.9 | 6.4 | 3.7 | 11.6 | 7.5 | 6.1 | 20.4 |
| AD 4 Hippo | 14.8 | 25.5 | 10.7 | 17.1 | 18.8 | 17.6 | 5.6 |
| AD 5 Hippo | 44.8 | 41.8 | 53.2 | 39.2 | 38.4 | 42.6 | 57.4 |
| AD 6 Hippo | 100.0 | 100.0 | 100.0 | 100.0 | 100.0 | 100.0 | 90.1 |
| Control 2 | 24.3 | 36.1 | 18.7 | 17.9 | 29.5 | 32.5 | 28.5 |
| Hippo | | | | | | | |
| Control 4 | 42.9 | 43.8 | 27.0 | 38.4 | 32.3 | 37.9 | 86.5 |
| Hippo | | | | | | | |
| Control | 14.2 | 11.4 | 4.6 | 10.2 | 6.0 | 6.4 | 0.0 |
| (Path) 3 | | | | | | | |
| Hippo | | | | | | | |
| AD 1 | 23.3 | 15.9 | 12.9 | 12.1 | 17.1 | 24.5 | 16.8 |
| Temporal | | | | | | | |
| Ctx | | | | | | | |
| AD 2 | 41.5 | 47.3 | 31.0 | 36.6 | 39.8 | 27.5 | 21.6 |
| Temporal | | | | | | | |
| Ctx | | | | | | | |
| AD 3 | 9.5 | 9.8 | 6.0 | 11.7 | 11.3 | 9.0 | 5.7 |
| Temporal | | | | | | | |
| Ctx | | | | | | | |
| AD 4 | 30.6 | 39.0 | 20.2 | 15.6 | 25.3 | 30.4 | 8.7 |
| Temporal | | | | | | | |
| Ctx | | | | | | | |
| AD 5 Inf | 45.4 | 37.1 | 39.2 | 43.8 | 36.3 | 41.8 | 73.7 |
| Temporal | | | | | | | |
| Ctx | | | | | | | |
| AD 5 Sup | 51.1 | 39.0 | 42.0 | 56.6 | 32.3 | 38.7 | 55.9 |
| Temporal | | | | | | | |
| Ctx | | | | | | | |
| AD 6 Inf | 38.2 | 59.9 | 49.3 | 40.9 | 46.7 | 47.6 | 76.8 |
| Temporal | | | | | | | |
| Ctx | | | | | | | |
| AD 6 Sup | 43.8 | 48.6 | 48.3 | 44.1 | 50.3 | 50.3 | 59.9 |
| Temporal | | | | | | | |
| Ctx | | | | | | | |
| Control 1 | 12.2 | 23.0 | 12.9 | 11.9 | 15.6 | 24.0 | 46.7 |
| Temporal | | | | | | | |
| Ctx | | | | | | | |
| Control 2 | 14.2 | 32.5 | 18.2 | 16.7 | 17.4 | 14.9 | 50.0 |
| Temporal | | | | | | | |
| Ctx | | | | | | | |
| Control 3 | 15.1 | 15.3 | 9.6 | 13.0 | 14.5 | 16.5 | 9.5 |
| Temporal | | | | | | | |
| Ctx | | | | | | | |
| Control 3 | 23.7 | 25.0 | 15.2 | 18.9 | 13.1 | 23.8 | 13.6 |
| Temporal | | | | | | | |
| Ctx | | | | | | | |
| Control | 26.1 | 47.0 | 27.0 | 32.5 | 30.6 | 39.8 | 46.0 |
| (Path) 1 | | | | | | | |
| Temporal | | | | | | | |
| Ctx | | | | | | | |
| Control | 24.5 | 25.9 | 16.0 | 19.5 | 20.4 | 24.8 | 0.0 |
| (Path) 2 | | | | | | | |
| Temporal | | | | | | | |
| Ctx | | | | | | | |
| Control | 11.7 | 16.0 | 7.5 | 12.9 | 10.9 | 11.9 | 31.0 |
| (Path) 3 | | | | | | | |
| Temporal | | | | | | | |
| Ctx | | | | | | | |
| Control | 21.9 | 27.4 | 17.1 | 19.8 | 18.2 | 21.6 | 39.5 |
| (Path) 4 | | | | | | | |
| Temporal | | | | | | | |
| Ctx | | | | | | | |
| AD 1 | 16.0 | 11.9 | 10.2 | 16.2 | 11.5 | 16.0 | 6.3 |
| Occipital Ctx | | | | | | | |
| AD 2 | 0.0 | 0.0 | 0.0 | 0.0 | 0.0 | 0.0 | 0.0 |
| Occipital Ctx | | | | | | | |
| (Missing) | | | | | | | |
| AD 3 | 10.7 | 6.0 | 6.4 | 11.7 | 8.8 | 10.2 | 4.9 |
| Occipital Ctx | | | | | | | |
| AD 4 | 18.9 | 23.7 | 13.0 | 12.6 | 17.9 | 18.6 | 11.1 |
| Occipital Ctx | | | | | | | |
| AD 5 | 24.8 | 28.3 | 25.3 | 16.7 | 22.5 | 22.7 | 42.3 |
| Occipital Ctx | | | | | | | |
| AD 6 | 20.6 | 31.9 | 20.2 | 17.8 | 17.0 | 22.1 | 14.8 |
| Occipital Ctx | | | | | | | |
| Control 1 | 9.5 | 14.4 | 6.0 | 11.3 | 8.7 | 7.2 | 8.8 |
| Occipital Ctx | | | | | | | |
| Control 2 | 31.9 | 42.6 | 26.4 | 24.8 | 33.2 | 29.3 | 82.4 |
| Occipital Ctx | | | | | | | |
| Control 3 | 18.8 | 13.0 | 10.7 | 16.4 | 17.1 | 19.2 | 8.8 |
| Occipital Ctx | | | | | | | |
| Control 4 | 18.2 | 17.0 | 12.0 | 12.1 | 12.6 | 13.6 | 24.0 |
| Occipital Ctx | | | | | | | |
| Control | 38.2 | 52.5 | 35.6 | 32.8 | 36.1 | 39.5 | 100.0 |
| (Path) 1 | | | | | | | |
| Occipital Ctx | | | | | | | |
| Control | 9.6 | 14.1 | 6.7 | 9.6 | 7.9 | 7.0 | 9.3 |
| (Path) 2 | | | | | | | |
| Occipital Ctx | | | | | | | |
| Control | 4.8 | 8.7 | 5.4 | 8.4 | 6.0 | 5.9 | 4.1 |
| (Path) 3 | | | | | | | |
| Occipital Ctx | | | | | | | |
| Control | 16.2 | 13.2 | 13.2 | 15.9 | 10.2 | 11.4 | 32.8 |
| (Path) 4 | | | | | | | |
| Occipital Ctx | | | | | | | |
| Control 1 | 14.4 | 21.9 | 8.8 | 15.2 | 16.3 | 15.7 | 9.2 |
| Parietal Ctx | | | | | | | |
| Control 2 | 32.8 | 28.9 | 34.4 | 39.5 | 28.3 | 37.1 | 28.1 |
| Parietal Ctx | | | | | | | |
| Control 3 | 20.6 | 19.8 | 11.5 | 14.5 | 8.7 | 10.8 | 9.1 |
| Parietal Ctx | | | | | | | |
| Control | 35.4 | 62.4 | 34.2 | 33.4 | 39.2 | 37.9 | 69.3 |
| (Path) 1 | | | | | | | |
| Parietal Ctx | | | | | | | |
| Control | 22.1 | 23.8 | 19.6 | 20.0 | 22.5 | 18.7 | 37.6 |
| (Path) 2 | | | | | | | |
| Parietal Ctx | | | | | | | |
| Control | 11.2 | 15.4 | 3.9 | 15.0 | 7.1 | 12.0 | 10.4 |
| (Path) 3 | | | | | | | |
| Parietal Ctx | | | | | | | |
| Control | 31.2 | 34.2 | 24.8 | 28.3 | 8.8 | 27.9 | 27.5 |
| (Path) 4 | | | | | | | |
| Parietal Ctx | | | | | | | |

TABLE AKL
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 113.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro; met) SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE AKM
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N4l7 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.O |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro; met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE AKN
General_screening_panel_v1.6

| | Rel. | Rel. | Rel. | Rel. | Rel. | Rel. | Rel. | Rel. |
|---|---|---|---|---|---|---|---|---|
| | Exp. (%) | Exp. (%) | Exp. (%) | Exp. (%) | Exp. (%) | Exp. (%) | Exp. (%) | Exp. (%) |
| | Ag6413, | Ag6424, | Ag6428, | Ag6430, | Ag6431, | Ag6431, | Ag6439, | Ag6964, |
| Tissue | Run | Run | Run | Run | Run | Run | Run | Run |
| Name | 277249371 | 277221719 | 277222439 | 277222443 | 277633568 | 278389390 | 277223175 | 278388946 |
| Adipose | 25.9 | 0.0 | 20.0 | 8.2 | 17.4 | 13.8 | 17.3 | 18.8 |
| Melanoma* | 0.5 | 0.0 | 2.0 | 0.5 | 0.8 | 0.9 | 0.4 | 0.7 |
| Hs688(A).T | | | | | | | | |
| Melanoma* | 2.7 | 0.0 | 4.1 | 0.6 | 2.5 | 2.2 | 2.9 | 2.4 |
| Hs688(B).T | | | | | | | | |
| Melanoma* | 0.3 | 0.0 | 0.7 | 0.7 | 0.4 | 0.4 | 0.4 | 0.7 |
| M14 | | | | | | | | |
| Melanoma* | 0.0 | 0.0 | 0.1 | 0.0 | 0.0 | 0.0 | 0.0 | 0.1 |
| LOXIMVI | | | | | | | | |
| Melanoma* | 15.2 | 0.0 | 30.4 | 22.5 | 18.2 | 14.6 | 18.3 | 15.9 |
| SK-MEL-5 | | | | | | | | |
| Squamous | 0.0 | 0.0 | 0.1 | 0.3 | 0.1 | 0.2 | 0.0 | 0.1 |
| cell | | | | | | | | |
| carcinoma | | | | | | | | |
| SCC-4 | | | | | | | | |
| Testis Pool | 5.2 | 0.0 | 8.8 | 4.2 | 10.4 | 9.0 | 9.1 | 9.9 |
| Prostate ca.* | 1.9 | 0.0 | 2.5 | 1.0 | 1.9 | 1.8 | 1.3 | 4.3 |
| (bone met) | | | | | | | | |
| PC-3 | | | | | | | | |
| Prostate Pool | 8.1 | 0.0 | 11.5 | 8.5 | 11.3 | 12.1 | 28.5 | 10.0 |
| Placenta | 0.5 | 0.0 | 0.7 | 0.1 | 0.1 | 0.1 | 0.5 | 0.4 |
| Uterus Pool | 2.2 | 0.0 | 4.5 | 2.6 | 4.6 | 4.5 | 5.3 | 4.1 |
| Ovarian ca. | 0.9 | 0.0 | 1.1 | 0.8 | 0.7 | 1.1 | 1.6 | 4.0 |
| OVCAR-3 | | | | | | | | |
| Ovarian ca. | 0.8 | 0.0 | 1.7 | 1.5 | 0.8 | 0.9 | 1.3 | 1.7 |
| SK-OV-3 | | | | | | | | |
| Ovarian ca. | 0.2 | 0.0 | 0.9 | 0.5 | 0.4 | 0.8 | 0.9 | 0.5 |
| OVCAR-4 | | | | | | | | |
| Ovarian ca. | 1.6 | 0.0 | 2.9 | 1.5 | 1.3 | 1.7 | 1.4 | 7.9 |
| OVCAR-5 | | | | | | | | |
| Ovarian ca. | 100.0 | 100.0 | 77.9 | 90.8 | 84.7 | 97.9 | 69.3 | 75.8 |
| IGROV-1 | | | | | | | | |
| Ovarian ca. | 13.6 | 5.6 | 14.0 | 11.9 | 15.6 | 14.6 | 17.3 | 16.7 |
| OVCAR-8 | | | | | | | | |
| Ovary | 2.7 | 0.0 | 5.2 | 2.1 | 3.1 | 2.3 | 2.8 | 2.4 |
| Breast ca. | 0.3 | 0.0 | 0.3 | 0.4 | 0.1 | 0.2 | 0.5 | 0.5 |
| MCF-7 | | | | | | | | |
| Breast ca. | 0.1 | 0.0 | 0.4 | 0.4 | 0.2 | 0.2 | 0.2 | 0.3 |
| MDA-MB- | | | | | | | | |
| 231 | | | | | | | | |
| Breast ca. | 0.5 | 0.0 | 0.5 | 0.3 | 0.1 | 0.5 | 0.6 | 0.4 |
| BT 549 | | | | | | | | |
| Breast ca. | 0.0 | 0.0 | 0.5 | 0.3 | 0.2 | 0.3 | 0.4 | 0.5 |
| T47D | | | | | | | | |
| Breast ca. | 0.6 | 0.0 | 0.7 | 0.7 | 0.6 | 0.6 | 0.6 | 0.8 |
| MDA-N | | | | | | | | |
| Breast Pool | 15.0 | 0.0 | 21.8 | 19.5 | 14.6 | 10.7 | 12.2 | 16.7 |
| Trachea | 4.5 | 0.0 | 8.4 | 2.9 | 4.8 | 4.2 | 4.7 | 5.6 |
| Lung | 2.8 | 0.0 | 2.3 | 1.3 | 4.2 | 3.2 | 3.9 | 5.1 |
| Fetal Lung | 3.9 | 0.0 | 9.1 | 4.0 | 5.0 | 4.8 | 5.3 | 6.1 |
| Lung ca. | 2.0 | 2.0 | 3.5 | 2.7 | 3.3 | 2.6 | 4.0 | 2.3 |
| NCI-N417 | | | | | | | | |
| Lung ca. LX- | 3.5 | 3.1 | 6.5 | 7.0 | 5.0 | 3.5 | 4.9 | 44.1 |
| 1 | | | | | | | | |
| Lung ca. | 0.1 | 0.0 | 0.3 | 0.5 | 0.1 | 0.2 | 0.1 | 0.1 |
| NCI-H146 | | | | | | | | |
| Lung ca. | 4.0 | 2.3 | 6.8 | 6.3 | 5.3 | 4.5 | 4.5 | 3.8 |
| SHP-77 | | | | | | | | |
| Lung ca. | 0.3 | 0.0 | 0.9 | 0.3 | 0.0 | 0.4 | 0.6 | 4.7 |
| A549 | | | | | | | | |
| Lung ca. | 0.2 | 0.0 | 0.9 | 0.7 | 0.6 | 0.3 | 0.4 | 0.5 |
| NCI-H526 | | | | | | | | |
| Lung ca. | 2.9 | 0.0 | 4.6 | 4.5 | 4.8 | 3.2 | 2.9 | 10.3 |
| NCI-H23 | | | | | | | | |
| Lung ca. | 0.0 | 0.0 | 0.2 | 0.2 | 0.1 | 0.3 | 0.0 | 0.3 |
| NCI-H460 | | | | | | | | |
| Lung ca. | 0.5 | 0.0 | 0.5 | 0.6 | 1.0 | 0.6 | 0.5 | 0.7 |
| HOP-62 | | | | | | | | |
| Lung ca. | 1.7 | 0.0 | 2.3 | 2.4 | 1.7 | 1.3 | 3.3 | 8.9 |
| NCI-H522 | | | | | | | | |
| Liver | 0.1 | 0.0 | 0.0 | 0.1 | 0.0 | 0.0 | 0.1 | 2.0 |
| Fetal Liver | 0.3 | 0.0 | 1.1 | 0.6 | 0.6 | 0.5 | 0.8 | 8.2 |
| Liver ca. | 0.1 | 0.0 | 0.2 | 0.1 | 0.0 | 0.2 | 0.1 | 2.4 |
| HepG2 | | | | | | | | |
| Kidney Pool | 27.9 | 6.5 | 47.0 | 34.9 | 33.9 | 28.1 | 43.2 | 32.8 |
| Fetal Kidney | 1.4 | 0.0 | 4.9 | 5.1 | 4.1 | 4.0 | 5.8 | 11.5 |
| Renal ca. | 0.2 | 0.0 | 0.2 | 0.2 | 0.3 | 0.1 | 0.3 | 0.9 |
| 786-0 | | | | | | | | |
| Renal ca. | 0.0 | 0.0 | 0.2 | 0.1 | 0.0 | 0.3 | 0.5 | 8.5 |
| A498 | | | | | | | | |
| Renal ca. | 1.5 | 0.0 | 2.5 | 0.7 | 1.7 | 1.5 | 1.2 | 2.5 |
| ACHN | | | | | | | | |
| Renal ca. | 0.3 | 0.0 | 0.5 | 0.3 | 0.2 | 0.2 | 0.6 | 0.3 |
| UO-31 | | | | | | | | |
| Renal ca. | 1.9 | 0.0 | 3.1 | 2.5 | 2.0 | 1.9 | 2.1 | 4.6 |
| TK-10 | | | | | | | | |
| Bladder | 4.2 | 0.0 | 5.9 | 3.0 | 5.5 | 5.1 | 8.3 | 6.7 |
| Gastric ca. | 0.9 | 0.0 | 1.7 | 1.7 | 0.9 | 1.2 | 1.1 | 6.7 |
| (liver met.) | | | | | | | | |
| NCI-N87 | | | | | | | | |
| Gastric ca. | 0.4 | 0.0 | 0.8 | 0.4 | 0.2 | 0.3 | 0.4 | 0.9 |
| KATO III | | | | | | | | |
| Colon ca. | 0.0 | 0.0 | 0.2 | 0.0 | 0.2 | 0.2 | 0.3 | 1.2 |
| SW-948 | | | | | | | | |
| Colon ca. | 20.9 | 9.5 | 41.8 | 39.0 | 27.0 | 23.3 | 23.0 | 33.7 |
| SW480 | | | | | | | | |
| Colon ca.* | 13.3 | 7.7 | 16.4 | 15.5 | 12.8 | 10.3 | 6.1 | 25.0 |
| (SW480 met) | | | | | | | | |
| SW620 | | | | | | | | |
| Colon ca. | 0.2 | 0.0 | 0.0 | 0.0 | 0.2 | 0.2 | 0.0 | 0.3 |
| HT29 | | | | | | | | |
| Colon ca. | 2.1 | 1.6 | 3.2 | 3.8 | 2.5 | 2.0 | 2.1 | 4.3 |
| HCT-116 | | | | | | | | |
| Colon ca. | 15.0 | 10.4 | 27.0 | 22.2 | 19.1 | 16.7 | 18.3 | 38.2 |
| CaCo-2 | | | | | | | | |
| Colon cancer | 9.0 | 0.0 | 11.0 | 6.5 | 11.9 | 7.6 | 7.7 | 20.4 |
| tissue | | | | | | | | |
| Colon ca. | 1.3 | 0.0 | 2.5 | 1.7 | 2.0 | 1.5 | 1.8 | 6.0 |
| SW1116 | | | | | | | | |
| Colon ca. | 0.1 | 0.0 | 0.3 | 0.2 | 0.2 | 0.0 | 0.2 | 0.8 |
| Colo-205 | | | | | | | | |
| Colon ca. | 0.8 | 0.0 | 1.4 | 1.3 | 1.5 | 1.5 | 1.4 | 2.6 |
| SW-48 | | | | | | | | |
| Colon Pool | 20.3 | 0.0 | 28.1 | 28.7 | 23.2 | 18.7 | 25.5 | 20.6 |
| Small | 14.0 | 0.0 | 17.1 | 10.5 | 11.2 | 13.0 | 12.8 | 10.4 |
| Intestine | | | | | | | | |
| Pool | | | | | | | | |
| Stomach | 8.1 | 0.0 | 14.3 | 6.2 | 9.5 | 9.3 | 8.5 | 10.7 |
| Pool | | | | | | | | |
| Bone | 6.8 | 0.0 | 14.3 | 11.3 | 10.2 | 8.7 | 18.7 | 12.5 |
| Marrow Pool | | | | | | | | |
| Fetal Heart | 10.1 | 0.0 | 25.5 | 24.3 | 24.5 | 21.8 | 33.7 | 20.7 |
| Heart Pool | 28.7 | 5.2 | 29.7 | 23.0 | 25.9 | 17.2 | 33.7 | 26.1 |
| Lymph Node | 17.6 | 0.0 | 33.7 | 30.4 | 22.1 | 23.7 | 19.9 | 24.7 |
| Pool | | | | | | | | |
| Fetal | 31.9 | 36.9 | 54.3 | 46.7 | 48.6 | 46.3 | 19.1 | 50.7 |
| Skeletal | | | | | | | | |
| Muscle | | | | | | | | |
| Skeletal | 17.4 | 12.3 | 29.3 | 21.5 | 29.5 | 25.9 | 22.1 | 32.3 |
| Muscle Pool | | | | | | | | |
| Spleen Pool | 0.9 | 0.0 | 1.9 | 2.0 | 2.0 | 1.7 | 2.7 | 3.1 |
| Thymus Pool | 4.4 | 0.0 | 10.4 | 7.5 | 8.1 | 9.4 | 7.7 | 7.0 |
| CNS cancer | 9.8 | 1.6 | 14.9 | 6.1 | 10.7 | 10.0 | 10.9 | 14.1 |
| (glio/astro) | | | | | | | | |
| U87-MG | | | | | | | | |
| CNS cancer | 3.5 | 0.0 | 4.7 | 2.9 | 3.8 | 3.1 | 3.8 | 5.8 |
| (glio/astro) | | | | | | | | |
| U-118-MG | | | | | | | | |
| CNS cancer | 1.9 | 0.0 | 2.6 | 1.7 | 2.1 | 1.0 | 1.4 | 2.6 |
| (neuro; met) | | | | | | | | |
| SK-N-AS | | | | | | | | |
| CNS cancer | 0.1 | 0.0 | 0.0 | 0.2 | 0.1 | 0.2 | 0.1 | 0.1 |
| (astro) SF- | | | | | | | | |
| 539 | | | | | | | | |
| CNS cancer | 8.1 | 1.9 | 14.9 | 5.9 | 6.5 | 10.0 | 11.7 | 9.7 |
| (astro) SNB- | | | | | | | | |
| 75 | | | | | | | | |
| CNS cancer | 79.6 | 84.1 | 100.0 | 100.0 | 100.0 | 100.0 | 100.0 | 100.0 |
| (glio) SNB- | | | | | | | | |
| 19 | | | | | | | | |
| CNS cancer | 8.2 | 1.8 | 11.3 | 9.0 | 8.0 | 7.8 | 8.2 | 14.8 |
| (glio) SF-295 | | | | | | | | |
| Brain | 3.7 | 2.3 | 7.7 | 6.9 | 6.2 | 4.8 | 8.0 | 5.3 |
| (Amygdala) | | | | | | | | |
| Pool | | | | | | | | |
| Brain | 12.0 | 6.6 | 19.8 | 11.1 | 10.7 | 9.7 | 8.8 | 9.7 |
| (cerebellum) | | | | | | | | |
| Brain (fetal) | 4.2 | 3.0 | 12.7 | 11.5 | 6.6 | 5.6 | 6.8 | 6.4 |
| Brain | 7.5 | 3.1 | 11.7 | 11.0 | 8.6 | 6.9 | 11.0 | 10.2 |
| (Hippocam- | | | | | | | | |
| pus) Pool | | | | | | | | |
| Cerebral | 9.7 | 1.7 | 11.0 | 7.5 | 7.5 | 0.7 | 11.6 | 8.7 |
| Cortex Pool | | | | | | | | |
| Brain | 7.4 | 1.8 | 11.7 | 8.5 | 10.4 | 4.7 | 10.0 | 9.3 |
| (Substantia | | | | | | | | |
| nigra) Pool | | | | | | | | |
| Brain | 7.6 | 0.0 | 13.2 | 10.0 | 9.3 | 0.2 | 9.7 | 8.7 |
| (Thalamus) | | | | | | | | |
| Pool | | | | | | | | |
| Brain | 6.1 | 0.0 | 10.6 | 8.0 | 5.8 | 0.3 | 5.6 | 8.7 |
| (whole) | | | | | | | | |
| Spinal Cord | 10.1 | 3.2 | 14.7 | 12.8 | 11.0 | 7.6 | 12.2 | 9.0 |
| Pool | | | | | | | | |
| Adrenal | 3.5 | 0.0 | 9.9 | 6.1 | 3.9 | 3.7 | 4.8 | 4.1 |
| Gland | | | | | | | | |
| Pituitary | 0.9 | 0.0 | 1.1 | 0.8 | 1.2 | 1.1 | 1.4 | 0.5 |
| gland Pool | | | | | | | | |
| Salivary | 0.9 | 0.0 | 1.8 | 1.1 | 1.3 | 0.9 | 1.1 | 1.0 |
| Gland | | | | | | | | |
| Thyroid | 2.0 | 0.0 | 3.1 | 0.8 | 2.5 | 2.5 | 1.9 | 2.3 |
| (female) | | | | | | | | |
| Pancreatic | 0.5 | 0.0 | 0.8 | 0.8 | 0.7 | 0.6 | 0.7 | 2.2 |
| ca. CAPAN2 | | | | | | | | |
| Pancreas | 1.2 | 0.0 | 2.0 | 1.1 | 1.1 | 1.6 | 3.2 | 2.3 |
| Pool | | | | | | | | |

TABLE AKO
Panel 4.1D

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag4983, | Ag6413, | Ag6428, | Ag6430, | Ag6431, | Ag6439, |
| | Run | Run | Run | Run | Run | Run |
| Tissue Name | 218623570 | 269239947 | 268767535 | 268767563 | 268767577 | 268760823 |
| Secondary Th1 act | 0.1 | 0.3 | 1.3 | 0.0 | 0.7 | 0.0 |
| Secondary Th2 act | 0.5 | 0.3 | 1.2 | 0.0 | 0.8 | 0.0 |
| Secondary Tr1 act | 0.0 | 0.0 | 0.0 | 0.0 | 0.7 | 0.0 |
| Secondary Th1 rest | 0.1 | 0.0 | 0.0 | 0.0 | 0.0 | 0.0 |
| Secondary Th2 rest | 0.3 | 0.0 | 0.0 | 0.0 | 0.0 | 0.0 |
| Secondary Tr1 rest | 0.1 | 0.3 | 0.4 | 0.0 | 0.0 | 0.0 |
| Primary Th1 act | 0.0 | 0.0 | 0.0 | 0.0 | 0.0 | 0.0 |
| Primary Th2 act | 0.2 | 0.4 | 0.3 | 0.0 | 0.4 | 0.0 |
| Primary Tr1 act | 0.1 | 0.0 | 0.7 | 0.0 | 0.7 | 0.0 |
| Primary Th1 rest | 0.0 | 0.0 | 0.1 | 0.0 | 0.3 | 1.2 |
| Primary Th2 rest | 0.0 | 0.0 | 0.4 | 0.0 | 0.2 | 0.0 |
| Primary Tr1 rest | 0.3 | 0.0 | 0.0 | 0.0 | 0.0 | 0.0 |
| CD45RA CD4 | 0.4 | 2.8 | 5.4 | 0.0 | 2.4 | 2.6 |
| lymphocyte act | | | | | | |
| CD45RO CD4 | 0.1 | 2.2 | 1.5 | 0.0 | 0.7 | 2.3 |
| lymphocyte act | | | | | | |
| CD8 lymphocyte act | 0.4 | 0.9 | 0.7 | 0.0 | 0.0 | 0.0 |
| Secondary CD8 | 0.1 | 0.0 | 8.8 | 0.0 | 0.0 | 0.0 |
| lymphocyte rest | | | | | | |
| Secondary CD8 | 0.0 | 0.1 | 0.4 | 0.0 | 0.3 | 0.0 |
| lymphocyte act | | | | | | |
| CD4 lymphocyte | 0.1 | 0.0 | 0.5 | 0.0 | 0.4 | 0.0 |
| none | | | | | | |
| 2ry | 0.3 | 0.2 | 0.0 | 0.0 | 0.0 | 1.2 |
| Th1/Th2/Tr1_anti- | | | | | | |
| CD95 CH11 | | | | | | |
| LAK cells rest | 5.6 | 5.0 | 11.8 | 0.1 | 3.8 | 15.2 |
| LAK cells IL-2 | 0.4 | 0.3 | 0.0 | 0.0 | 0.0 | 0.0 |
| LAK cells IL-2 + IL- | 0.2 | 0.0 | 0.0 | 0.0 | 0.0 | 0.0 |
| 12 | | | | | | |
| LAK cells IL-2 + IFN | 0.1 | 0.3 | 0.0 | 0.0 | 0.0 | 0.0 |
| gamma | | | | | | |
| LAK cells IL-2 + IL- | 0.0 | 0.0 | 0.0 | 0.0 | 0.0 | 0.0 |
| 18 | | | | | | |
| LAK cells | 4.5 | 4.0 | 15.1 | 0.1 | 6.3 | 9.0 |
| PMA/ionomycin | | | | | | |
| NK Cells IL-2 rest | 0.9 | 0.1 | 3.4 | 0.0 | 2.5 | 1.4 |
| Two Way MLR 3 | 1.4 | 1.1 | 2.2 | 0.0 | 1.3 | 1.4 |
| day | | | | | | |
| Two Way MLR 5 | 4.5 | 0.9 | 0.8 | 0.0 | 0.9 | 0.0 |
| day | | | | | | |
| Two Way MLR 7 | 2.3 | 0.7 | 1.1 | 0.0 | 2.6 | 3.7 |
| day | | | | | | |
| PBMC rest | 0.1 | 0.0 | 0.0 | 0.0 | 0.0 | 0.0 |
| PBMC PWM | 0.6 | 0.0 | 1.3 | 0.0 | 0.0 | 0.0 |
| PBMC PHA-L | 0.3 | 0.2 | 0.6 | 0.0 | 0.7 | 0.0 |
| Ramos (B cell) none | 0.1 | 0.0 | 0.0 | 0.0 | 0.0 | 0.0 |
| Ramos (B cell) | 0.0 | 0.0 | 0.7 | 0.0 | 0.2 | 0.0 |
| ionomycin | | | | | | |
| B lymphocytes | 0.5 | 0.0 | 0.0 | 0.0 | 0.0 | 0.0 |
| PWM | | | | | | |
| B lymphocytes | 0.2 | 0.0 | 0.9 | 0.0 | 0.0 | 0.0 |
| CD40L and IL-4 | | | | | | |
| EOL-1 dbcAMP | 3.7 | 2.6 | 29.1 | 0.1 | 8.1 | 68.8 |
| EOL-1 dbcAMP | 1.6 | 0.7 | 0.0 | 0.0 | 2.7 | 1.8 |
| PMA/ionomycin | | | | | | |
| Dendritic cells none | 5.6 | 3.1 | 4.1 | 0.0 | 5.3 | 0.0 |
| Dendritic cells LPS | 1.6 | 0.3 | 1.0 | 0.0 | 0.7 | 0.0 |
| Dendritic cells anti- | 2.0 | 1.6 | 0.5 | 0.0 | 0.2 | 0.0 |
| CD40 | | | | | | |
| Monocytes rest | 0.2 | 0.0 | 0.4 | 0.0 | 0.0 | 0.0 |
| Monocytes LPS | 2.2 | 3.3 | 5.7 | 0.0 | 1.8 | 2.6 |
| Macrophages rest | 0.9 | 1.8 | 0.6 | 0.0 | 0.6 | 0.0 |
| Macrophages LPS | 7.5 | 4.0 | 5.4 | 0.1 | 6.3 | 9.2 |
| HUVEC none | 0.1 | 0.0 | 0.0 | 0.0 | 0.0 | 0.0 |
| HUVEC starved | 0.0 | 0.0 | 0.0 | 0.0 | 0.3 | 0.0 |
| HUVEC IL-1beta | 0.0 | 0.0 | 0.0 | 0.0 | 0.5 | 0.0 |
| HUVEC IFN | 0.2 | 0.0 | 0.0 | 0.0 | 0.0 | 0.0 |
| gamma | | | | | | |
| HUVEC TNF alpha + | 0.0 | 0.0 | 0.0 | 0.0 | 0.0 | 0.0 |
| IFN gamma | | | | | | |
| HUVEC TNF alpha + | 0.6 | 0.0 | 0.0 | 0.0 | 0.4 | 0.0 |
| IL4 | | | | | | |
| HUVEC IL-11 | 0.0 | 0.0 | 0.4 | 0.0 | 0.3 | 0.0 |
| Lung Microvascular | 0.2 | 0.3 | 0.4 | 0.0 | 0.0 | 0.0 |
| EC none | | | | | | |
| Lung Microvascular | 0.1 | 0.0 | 0.0 | 0.0 | 0.0 | 0.0 |
| EC TNFalpha + IL- | | | | | | |
| 1beta | | | | | | |
| Microvascular | 0.1 | 0.0 | 0.0 | 0.0 | 0.0 | 0.0 |
| Dermal EC none | | | | | | |
| Microsvasular | 0.1 | 0.0 | 0.0 | 0.0 | 0.0 | 0.0 |
| Dermal EC | | | | | | |
| TNFalpha + IL- | | | | | | |
| 1beta | | | | | | |
| Bronchial | 0.0 | 0.0 | 0.0 | 0.0 | 0.0 | 0.0 |
| epithelium | | | | | | |
| TNFalpha + IL1beta | | | | | | |
| Small airway | 0.0 | 0.0 | 0.0 | 0.0 | 0.0 | 0.0 |
| epithelium none | | | | | | |
| Small airway | 0.3 | 0.0 | 0.0 | 0.0 | 0.0 | 0.0 |
| epithelium | | | | | | |
| TNFalpha + IL- | | | | | | |
| 1beta | | | | | | |
| Coronery artery | 0.1 | 0.6 | 0.0 | 0.0 | 0.0 | 0.0 |
| SMC rest | | | | | | |
| Coronery artery | 0.4 | 0.9 | 0.3 | 0.0 | 1.5 | 0.0 |
| SMC TNFalpha + | | | | | | |
| IL-1beta | | | | | | |
| Astrocytes rest | 67.8 | 97.3 | 100.0 | 12.0 | 100.0 | 100.0 |
| Astrocytes | 100.0 | 100.0 | 97.3 | 100.0 | 74.7 | 95.9 |
| TNFalpha + IL- | | | | | | |
| 1beta | | | | | | |
| KU-812 (Basophil) | 0.1 | 0.0 | 0.0 | 0.0 | 0.4 | 0.0 |
| rest | | | | | | |
| KU-812 (Basophil) | 0.0 | 0.0 | 0.0 | 0.0 | 0.0 | 0.0 |
| PMA/ionomycin | | | | | | |
| CCD1106 | 0.2 | 0.0 | 0.0 | 0.0 | 0.8 | 0.0 |
| (Keratinocytes) none | | | | | | |
| CCD1106 | 0.3 | 0.0 | 0.0 | 0.0 | 0.0 | 0.0 |
| (Keratinocytes) | | | | | | |
| TNFalpha + IL- | | | | | | |
| 1beta | | | | | | |
| Liver cirrhosis | 2.3 | 7.2 | 2.6 | 0.0 | 6.7 | 8.5 |
| NCI-H292 none | 0.3 | 0.3 | 1.7 | 0.0 | 0.6 | 0.0 |
| NCI-H292 IL-4 | 0.3 | 0.0 | 0.0 | 0.0 | 0.5 | 0.0 |
| NCI-H292 IL-9 | 0.3 | 0.0 | 0.7 | 0.0 | 0.5 | 0.0 |
| NCI-H292 IL-13 | 0.6 | 0.6 | 0.9 | 0.0 | 0.9 | 0.0 |
| NCI-H292 IFN | 0.2 | 0.0 | 0.5 | 0.0 | 0.6 | 0.0 |
| gamma | | | | | | |
| HPAEC none | 0.0 | 0.3 | 0.0 | 0.0 | 0.0 | 0.0 |
| HPAEC TNF alpha + | 0.0 | 0.3 | 0.0 | 0.0 | 0.0 | 0.0 |
| IL-1 beta | | | | | | |
| Lung fibroblast none | 29.7 | 62.9 | 95.9 | 0.2 | 65.5 | 94.0 |
| Lung fibroblast TNF | 16.0 | 36.9 | 48.6 | 0.1 | 39.8 | 62.9 |
| alpha + IL-1 beta | | | | | | |
| Lung fibroblast IL-4 | 26.1 | 28.7 | 27.4 | 0.1 | 21.2 | 34.9 |
| Lung fibroblast IL-9 | 28.5 | 42.0 | 24.0 | 0.1 | 26.8 | 96.6 |
| Lung fibroblast IL- | 31.6 | 14.6 | 11.9 | 0.0 | 10.4 | 13.4 |
| 13 | | | | | | |
| Lung fibroblast IFN | 20.4 | 32.8 | 55.9 | 0.2 | 46.3 | 89.5 |
| gamma | | | | | | |
| Dermal fibroblast | 2.5 | 2.9 | 6.0 | 0.0 | 6.3 | 4.1 |
| CCD1070 rest | | | | | | |
| Dermal fibroblast | 1.1 | 1.3 | 2.7 | 0.0 | 0.8 | 2.3 |
| CCD1070 TNF | | | | | | |
| alpha | | | | | | |
| Dermal fibroblast | 1.9 | 2.9 | 5.6 | 0.0 | 1.3 | 0.0 |
| CCD1070 IL-1 beta | | | | | | |
| Dermal fibroblast | 9.3 | 20.3 | 30.6 | 0.1 | 20.2 | 26.6 |
| IFN gamma | | | | | | |
| Dermal fibroblast | 10.7 | 14.6 | 30.8 | 0.1 | 19.8 | 25.5 |
| IL-4 | | | | | | |
| Dermal Fibroblasts | 24.8 | 42.3 | 54.3 | 0.1 | 46.7 | 47.3 |
| rest | | | | | | |
| Neutrophils | 0.7 | 0.0 | 0.9 | 0.0 | 0.4 | 0.0 |
| TNFa + LPS | | | | | | |
| Neutrophils rest | 0.1 | 0.0 | 0.0 | 0.0 | 0.3 | 0.0 |
| Colon | 7.9 | 4.7 | 4.6 | 0.0 | 9.5 | 8.4 |
| Lung | 2.2 | 1.2 | 2.8 | 0.0 | 4.6 | 2.1 |
| Thymus | 3.1 | 0.8 | 0.0 | 0.0 | 0.4 | 2.4 |
| Kidney | 4.2 | 4.4 | 7.8 | 0.1 | 9.7 | 5.2 |

TABLE AKP
general oncology screening panel_v_2.4

| | Rel. Exp. (%) | Rel. Exp. (%) |
|---|---|---|
| | Ag4983, Run | Ag6442, Run |
| Tissue Name | 260281959 | 264979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell carcinoma 3 | 5.6 | 8.3 |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate adenocarcinoma 1 | 9.2 | 7.5 |
| Prostate adenocarcinoma 2 | 3.5 | 8.0 |
| Prostate adenocarcinoma 3 | 14.3 | 9.0 |
| Prostate adenocarcinoma 4 | 16.4 | 9.1 |
| Prostate NAT 5 | 16.8 | 9.9 |
| Prostate adenocarcinoma 6 | 3.2 | 7.7 |
| Prostate adenocarcinoma 7 | 9.2 | 17.3 |
| Prostate adenocarcinoma 8 | 3.0 | 0.0 |
| Prostate adenocarcinoma 9 | 27.0 | 33.9 |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0 Summary: Ag4983/Ag6413/Ag6428/Ag6430/Ag6431/Ag6439/Ag6442 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

General_screening_panel_v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening_panel_v1.6

Summary: Ag6413/Ag6424/Ag6428/Ag6430/Ag6431/Ag6439/Ag6964 Eight experiments with seven different probe and primer sets are in very good agreement. Highest expression of this gene is detected in a ovarian cancer IGROV-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6429 Expression of this gene is low/undetectable (CTs>35) across all of the samples on this panel (data not shown).

Panel 4.1D

Summary: Ag4983/Ag6413/Ag6428/Ag6430/Ag6431/Ag6439/Ag6442 Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

Ag6424 Expression of this gene is low/undetectable (CTs>35) across all of the samples on this panel (data not shown).

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AL. CG56054-03: Integrin Alpha 7-Like Protein.

Expression of gene CG56054-03 was assessed using the primer-probe sets Ag6424, Ag6425, Ag6428, Ag6430 and Ag6432, described in Tables ALA, ALB, ALC, ALD and ALE. Results of the RTQ-PCR runs are shown in Tables ALF, ALG and ALH.

TABLE ALA

Probe Name Ag6424

| | | | | |
|---|---|---|---|---|
| Primers | Sequences | Length | Start Position | SEQ ID No |

| | | | | |
|---|---|---|---|---|
| Forward | 5′-ttgggttctgccagca-3′ | 16 | 742 | 444 |
| Probe | TET-5′-cacagctgccgccttctccc-3′-TAMRA | 20 | 761 | 445 |
| Reverse | 5′-aaaagcaaccccttccaa-3′ | 18 | 824 | 446 |

TABLE ALB

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 1981 | 447 |
| Probe | TET-5′-catcccgagctgggcccc-3′-TAMRA | 18 | 2013 | 448 |
| Reverse | 5′-gccctggatgcccat-3′ | 15 | 2032 | 449 |

TABLE ALC

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 120 | 1394 | 450 |
| Probe | TET-5′-ccttcacaggtgctggagggc-3′-TAMRA | 21 | 1434 | 451 |
| Reverse | 5′-agggagtagccgaagctct-3′ | 19 | 1471 | 452 |

TABLE ALD

Probe Name Ag6430

| | | | Start | |
|---|---|---|---|---|
| Primers | Sequences | Length | Position | SEQ ID No |
| Forward | 5′-gtgaccaacattgatagctcaga-3′ | 23 | 843 | 453 |
| Probe | TET-5′-ccccgaccagctggtgtataaaactttg-3′-TAMRA | 28 | 866 | 454 |
| Reverse | 5′-gggagccggtcagcaa-3′ | 15 | 899 | 455 |

TABLE ALE

Probe Name Ag6432

| | | | | |
|---|---|---|---|---|
| Primers | Sequences | Length | Start | SEQ ID No |

| | | | | |
|---|---|---|---|---|
| Forward | 5′-gaccttgtcctacagtctccagac-3′ | 24 | 1934 | 456 |
| Probe | TET-5′-tgcacaccccatcctggctgct-3′-TAMRA | 22 | 1985 | 457 |
| Reverse | 5′-gctcgggatgcccgt-3′ | 15 | 2008 | 458 |

TABLE ALF
CNS_neurodegeneration_v1.0

| | Rel. Exp. (%) | Rel. Exp. (%) |
|---|---|---|
| | Ag6428, Run | Ag6430, Run |
| Tissue Name | 266937081 | 266937085 |
| AD 1 Hippo | 18.0 | 20.0 |
| AD 2 Hippo | 32.3 | 48.0 |
| AD 3 Hippo | 3.7 | 11.6 |
| AD 4 Hippo | 10.7 | 17.1 |
| AD 5 hippo | 53.2 | 39.2 |
| AD 6 Hippo | 100.0 | 100.0 |
| Control 2 Hippo | 18.7 | 17.9 |
| Control 4 Hippo | 27.0 | 38.4 |
| Control (Path) 3 Hippo | 4.6 | 10.2 |
| AD 1 Temporal Ctx | 12.9 | 12.1 |
| AD 2 Temporal Ctx | 31.0 | 36.6 |
| AD 3 Temporal Ctx | 6.0 | 11.7 |
| AD 4 Temporal Ctx | 20.2 | 15.6 |
| AD 5 Inf Temporal Ctx | 39.2 | 43.8 |
| AD 5 Sup Temporal Ctx | 42.0 | 56.6 |
| AD 6 Inf Temporal Ctx | 49.3 | 40.9 |
| AD 6 Sup Temporal Ctx | 48.3 | 44.1 |
| Control 1 Temporal Ctx | 12.9 | 11.9 |
| Control 2 Temporal Ctx | 18.2 | 16.7 |
| Control 3 Temporal Ctx | 9.6 | 13.0 |
| Control 4 Temporal Ctx | 15.2 | 18.9 |
| Control (Path) 1 | 27.0 | 32.5 |
| Temporal Ctx | | |
| Control (Path) 2 | 16.0 | 19.5 |
| Temporal Ctx | | |
| Control (Path) 3 | 7.5 | 12.9 |
| Temporal Ctx | | |
| Control (Path) 4 | 17.1 | 19.8 |
| Temporal Ctx | | |
| AD 1 Occipital Ctx | 10.2 | 16.2 |
| AD 2 Occipital Ctx | 0.0 | 0.0 |
| (Missing) | | |
| AD 3 Occipital Ctx | 6.4 | 11.7 |
| AD 4 Occipital Ctx | 13.0 | 12.6 |
| AD 5 Occipital Ctx | 25.3 | 16.7 |
| AD 6 Occipital Ctx | 20.2 | 17.8 |
| Control 1 Occipital Ctx | 6.0 | 11.3 |
| Control 2 Occipital Ctx | 26.4 | 24.8 |
| Control 3 Occipital Ctx | 10.7 | 16.4 |
| Control 4 Occipital Ctx | 12.0 | 12.1 |
| Control (Path) 1 | 35.6 | 32.8 |
| Occipital Ctx | | |
| Control (Path) 2 | 6.7 | 9.6 |
| Occipital Ctx | | |
| Control (Path) 3 | 5.4 | 8.4 |
| Occipital Ctx | | |
| Control (Path) 4 | 13.2 | 15.9 |
| Occipital Ctx | | |
| Control 1 Parietal Ctx | 8.8 | 15.2 |
| Control 2 Parietal Ctx | 34.4 | 39.5 |
| Control 3 Parietal Ctx | 11.5 | 14.5 |
| Control (Path) 1 | 34.2 | 33.4 |
| Parietal Ctx | | |
| Control (Path) 2 | 19.6 | 20.0 |
| Parietal Ctx | | |
| Control (Path) 3 | 3.9 | 15.0 |
| Parietal Ctx | | |
| Control (Path) 4 | 24.8 | 28.3 |
| Parietal Ctx | | |

TABLE ALG
General_screening_panel_v1.6

| | Rel. Exp. | Rel. Exp. | Rel. Exp. | Rel. Exp. |
|---|---|---|---|---|
| | (%) Ag6424, | (%) Ag6425, | (%) Ag6428, | (%) Ag6430, |
| | Run | Run | Run | Run |
| Tissue Name | 277221719 | 277221721 | 277222439 | 277222443 |
| Adipose | 0.0 | 2.6 | 20.0 | 8.2 |
| Melanoma* Hs688(A).T | 0.0 | 0.0 | 2.0 | 0.5 |
| Melanoma* Hs688(B).T | 0.0 | 0.2 | 4.1 | 0.6 |
| Melanoma* M14 | 0.0 | 0.0 | 0.7 | 0.7 |
| Melanoma* LOXIMVI | 0.0 | 0.0 | 0.1 | 0.0 |
| Melanoma* SK-MEL-5 | 0.0 | 2.2 | 30.4 | 22.5 |
| Squamous cell carcinoma SCC-4 | 0.0 | 0.0 | 0.1 | 0.3 |
| Testis Pool | 0.0 | 3.5 | 8.8 | 4.2 |
| Prostate ca.* (bone met) PC-3 | 0.0 | 0.5 | 2.5 | 1.0 |
| Prostate Pool | 0.0 | 1.0 | 11.5 | 8.5 |
| Placenta | 0.0 | 0.0 | 0.7 | 0.1 |
| Uterus Pool | 0.0 | 1.5 | 4.5 | 2.6 |
| Ovarian ca. OVCAR-3 | 0.0 | 0.3 | 1.1 | 0.8 |
| Ovarian ca. SK-OV-3 | 0.0 | 0.2 | 1.7 | 1.5 |
| Ovarian ca. OVCAR-4 | 0.0 | 0.0 | 0.9 | 0.5 |
| Ovarian ca. OVCAR-5 | 0.0 | 1.3 | 2.9 | 1.5 |
| Ovarian ca. IGROV-1 | 100.0 | 100.0 | 77.9 | 90.8 |
| Ovarian ca. OVCAR-8 | 5.6 | 21.9 | 14.0 | 11.9 |
| Ovary | 0.0 | 0.3 | 5.2 | 2.1 |
| Breast ca. MCF-7 | 0.0 | 0.0 | 0.3 | 0.4 |
| Breast ca. MDA-MB-231 | 0.0 | 0.0 | 0.4 | 0.4 |
| Breast ca. BT 549 | 0.0 | 0.0 | 0.5 | 0.3 |
| Breast ca. T47D | 0.0 | 0.0 | 0.5 | 0.3 |
| Breast ca. MDA-N | 0.0 | 0.0 | 0.7 | 0.7 |
| Breast Pool | 0.0 | 4.1 | 21.8 | 19.5 |
| Trachea | 0.0 | 0.7 | 8.4 | 2.9 |
| Lung | 0.0 | 0.7 | 2.3 | 1.3 |
| Fetal Lung | 0.0 | 0.3 | 9.1 | 4.0 |
| Lung ca. NCI-N417 | 2.0 | 0.9 | 3.5 | 2.7 |
| Lung ca. LX-1 | 3.1 | 2.7 | 6.5 | 7.0 |
| Lung ca. NCI-H146 | 0.0 | 0.0 | 0.3 | 0.5 |
| Lung ca. SHP-77 | 2.3 | 0.4 | 6.8 | 6.3 |
| Lung ca. A549 | 0.0 | 2.6 | 0.9 | 0.3 |
| Lung ca. NCI-H526 | 0.0 | 0.0 | 0.9 | 0.7 |
| Lung ca. NCI-H23 | 0.0 | 1.0 | 4.6 | 4.5 |
| Lung ca. NCI-H460 | 0.0 | 0.0 | 0.2 | 0.2 |
| Lung ca. HOP-62 | 0.0 | 0.0 | 0.5 | 0.6 |
| Lung ca. NCI-H522 | 0.0 | 0.6 | 2.3 | 2.4 |
| Liver | 0.0 | 0.0 | 0.0 | 0.1 |
| Fetal Liver | 0.0 | 0.3 | 1.1 | 0.6 |
| Liver ca. HepG2 | 0.0 | 0.3 | 0.2 | 0.1 |
| Kidney Pool | 6.5 | 0.0 | 47.0 | 34.9 |
| Fetal Kidney | 0.0 | 0.0 | 4.9 | 5.1 |
| Renal ca. 786-0 | 0.0 | 0.0 | 0.2 | 0.2 |
| Renal ca. A498 | 0.0 | 1.8 | 0.2 | 0.1 |
| Renal ca. ACHN | 0.0 | 0.5 | 2.5 | 0.7 |
| Renal ca. UO-31 | 0.0 | 0.0 | 0.5 | 0.3 |
| Renal ca. TK-10 | 0.0 | 0.4 | 3.1 | 2.5 |
| Bladder | 0.0 | 0.0 | 5.9 | 3.0 |
| Gastric ca. (liver met.) NCI-N87 | 0.0 | 0.0 | 1.7 | 1.7 |
| Gastric ca. KATO III | 0.0 | 0.5 | 0.8 | 0.4 |
| Colon ca. SW-948 | 0.0 | 1.5 | 0.2 | 0.0 |
| Colon ca. SW480 | 9.5 | 5.2 | 41.8 | 39.0 |
| Colon ca.* (SW480 met) SW620 | 7.7 | 4.8 | 16.4 | 15.5 |
| Colon ca. HT29 | 0.0 | 0.0 | 0.0 | 0.0 |
| Colon ca. HCT-116 | 1.6 | 0.2 | 3.2 | 3.8 |
| Colon ca. CaCo-2 | 10.4 | 3.6 | 27.0 | 22.2 |
| Colon cancer tissue | 0.0 | 3.3 | 11.0 | 6.5 |
| Colon ca. SW1116 | 0.0 | 3.0 | 2.5 | 1.7 |
| Colon ca. Colo-205 | 0.0 | 0.4 | 0.3 | 0.2 |
| Colon ca. SW-48 | 0.0 | 3.6 | 1.4 | 1.3 |
| Colon Pool | 0.0 | 5.0 | 28.1 | 28.7 |
| Small Intestine Pool | 0.0 | 1.7 | 17.1 | 10.5 |
| Stomach Pool | 0.0 | 2.3 | 14.3 | 6.2 |
| Bone Marrow Pool | 0.0 | 1.6 | 14.3 | 11.3 |
| Fetal Heart | 0.0 | 2.3 | 25.5 | 24.3 |
| Heart Pool | 5.2 | 7.0 | 29.7 | 23.0 |
| Lymph Node Pool | 0.0 | 6.1 | 33.7 | 30.4 |
| Fetal Skeletal Muscle | 36.9 | 5.2 | 54.3 | 46.7 |
| Skeletal Muscle Pool | 12.3 | 9.2 | 29.3 | 21.5 |
| Spleen Pool | 0.0 | 0.0 | 1.9 | 2.0 |
| Thymus Pool | 0.0 | 2.0 | 10.4 | 7.5 |
| CNS cancer (glio/astro) U87-MG | 1.6 | 1.5 | 14.9 | 6.1 |
| CNS cancer (glio/astro) U-118-MG | 0.0 | 0.3 | 4.7 | 2.9 |
| CNS cancer (neuro; met) SK-N-AS | 0.0 | 0.0 | 2.6 | 1.7 |
| CNS cancer (astro) SF-539 | 0.0 | 0.0 | 0.0 | 0.2 |
| CNS cancer (astro) SNB-75 | 1.9 | 1.1 | 14.9 | 5.9 |
| CNS cancer (glio) SNB-19 | 84.1 | 79.0 | 100.0 | 100.0 |
| CNS cancer (glio) SF-295 | 1.8 | 0.0 | 11.3 | 9.0 |
| Brain (Amygdala) Pool | 2.3 | 0.8 | 7.7 | 6.9 |
| Brain (cerebellum) | 6.6 | 0.4 | 19.8 | 11.1 |
| Brain (fetal) | 3.0 | 0.7 | 12.7 | 11.5 |
| Brain (Hippocampus) Pool | 3.1 | 3.2 | 11.7 | 11.0 |
| Cerebral Cortex Pool | 1.7 | 0.6 | 11.0 | 7.5 |
| Brain (Substantia nigra) Pool | 1.8 | 2.2 | 11.7 | 8.5 |
| Brain (Thalamus) Pool | 0.0 | 2.7 | 13.2 | 10.0 |
| Brain (whole) | 0.0 | 0.4 | 10.6 | 8.0 |
| Spinal Cord Pool | 3.2 | 2.3 | 14.7 | 12.8 |
| Adrenal Gland | 0.0 | 0.3 | 9.9 | 6.1 |
| Pituitary gland Pool | 0.0 | 0.0 | 1.1 | 0.8 |
| Salivary Gland | 0.0 | 0.0 | 1.8 | 1.1 |
| Thyroid (female) | 0.0 | 0.3 | 3.1 | 0.8 |
| Pancreatic ca. CAPAN2 | 0.0 | 0.0 | 0.8 | 0.8 |
| Pancreas Pool | 0.0 | 0.0 | 2.0 | 1.1 |

TABLE ALH
Panel 4.1D

| | Rel. Exp. (%) | Rel. Exp. (%) | Rel. Exp. (%) |
|---|---|---|---|
| | Ag6425, Run | Ag6428, Run | Ag6430,Run |
| Tissue Name | 268713999 | 268767535 | 268767563 |
| Secondary Th1 act | 0.0 | 1.3 | 0.0 |
| Secondary Th2 act | 0.0 | 1.2 | 0.0 |
| Secondary Tr1 act | 0.0 | 0.0 | 0.0 |
| Secondary Th1 rest | 0.0 | 0.0 | 0.0 |
| Secondary Th2 rest | 0.0 | 0.0 | 0.0 |
| Secondary Tr1 rest | 0.0 | 0.4 | 0.0 |
| Primary Th1 act | 0.0 | 0.0 | 0.0 |
| Primary Th2 act | 0.0 | 0.3 | 0.0 |
| Primary Tr1 act | 0.0 | 0.7 | 0.0 |
| Primary Th1 rest | 0.0 | 0.1 | 0.0 |
| Primary Th2 rest | 0.0 | 0.4 | 0.0 |
| Primary Tr1 rest | 0.0 | 0.0 | 0.0 |
| CD45RA CD4 lymphocyte act | 0.0 | 5.4 | 0.0 |
| CD45RO CD4 lymphocyte act | 0.0 | 1.5 | 0.0 |
| CD8 lymphocyte act | 0.0 | 0.7 | 0.0 |
| Secondary CD8 lymphocyterest | 0.0 | 8.8 | 0.0 |
| Secondary CD8 lymphocyteact | 0.0 | 0.4 | 0.0 |
| CD4 lymphocyte none | 0.0 | 0.5 | 0.0 |
| 2ry Th1/Th2/Tr1_anti-CD95 CH11 | 0.0 | 0.0 | 0.0 |
| LAK cells rest | 2.7 | 11.8 | 0.1 |
| LAK cells IL-2 | 0.0 | 0.0 | 0.0 |
| LAK cells IL-2 + IL-12 | 0.0 | 0.0 | 0.0 |
| LAK cells IL-2 + IFN gamma | 0.0 | 0.0 | 0.0 |
| LAK cells IL-2 + IL-18 | 0.0 | 0.0 | 0.0 |
| LAK cells PMA/ionomycin | 15.7 | 15.1 | 0.1 |
| NK Cells IL-2 rest | 0.0 | 3.4 | 0.0 |
| Two Way MLR 3 day | 0.0 | 2.2 | 0.0 |
| Two Way MLR 5 day | 0.0 | 0.8 | 0.0 |
| Two Way MLR 7 day | 13.2 | 1.1 | 0.0 |
| PBMC rest | 0.0 | 0.0 | 0.0 |
| PBMC PWM | 0.0 | 1.3 | 0.0 |
| PBMC PHA-L | 0.0 | 0.6 | 0.0 |
| Ramos (B cell) none | 0.0 | 0.0 | 0.0 |
| Ramos (B cell) ionomycin | 0.0 | 0.7 | 0.0 |
| B lymphocytes PWM | 0.0 | 0.0 | 0.0 |
| B lymphocytes CD40L and IL-4 | 0.0 | 0.9 | 0.0 |
| EOL-1 dbcAMP | 9.1 | 29.1 | 0.1 |
| EOL-1 dbcAMP PMA/ionomycin | 0.0 | 0.0 | 0.0 |
| Dendritic cells none | 13.8 | 4.1 | 0.0 |
| Dendritic cells LPS | 0.0 | 1.0 | 0.0 |
| Dendritic cells anti-CD40 | 3.3 | 0.5 | 0.0 |
| Monocytes rest | 0.0 | 0.4 | 0.0 |
| Monocytes LPS | 0.0 | 5.7 | 0.0 |
| Macrophages rest | 0.0 | 0.6 | 0.0 |
| Macrophages LPS | 0.0 | 5.4 | 0.1 |
| HUVEC none | 0.0 | 0.0 | 0.0 |
| HUVEC starved | 0.0 | 0.0 | 0.0 |
| HUVEC IL-1beta | 0.0 | 0.0 | 0.0 |
| HUVEC IFN gamma | 0.0 | 0.0 | 0.0 |
| HUVEC TNF alpha + IFN gamma | 0.0 | 0.0 | 0.0 |
| HUVEC TNF alpha + IL4 | 0.0 | 0.0 | 0.0 |
| HUVEC IL-11 | 0.0 | 0.4 | 0.0 |
| Lung Microvascular EC none | 0.0 | 0.4 | 0.0 |
| Lung Microvascular EC | 0.0 | 0.0 | 0.0 |
| TNFalpha + IL-1beta | | | |
| Microvascular Dermal EC | 0.0 | 0.0 | 0.0 |
| none | | | |
| Microsvasular Dermal EC | 0.0 | 0.0 | 0.0 |
| TNFalpha + IL-1beta | | | |
| Bronchial epithelium | 0.0 | 0.0 | 0.0 |
| TNFalpha + IL1beta | | | |
| Small airway epithelium none | 0.0 | 0.0 | 0.0 |
| Small airway epithelium | 0.0 | 0.0 | 0.0 |
| TNFalpha + IL-1beta | | | |
| Coronery artery SMC rest | 0.0 | 0.0 | 0.0 |
| Coronery artery SMC | 6.2 | 0.3 | 0.0 |
| TNFalpha + IL-1beta | | | |
| Astrocytes rest | 100.0 | 100.0 | 12.0 |
| Astrocytes TNFalpha + IL-1beta | 74.2 | 97.3 | 100.0 |
| KU-812 (Basophil) rest | 0.0 | 0.0 | 0.0 |
| KU-812 (Basophil) PMA/ionomycin | 0.0 | 0.0 | 0.0 |
| CCD1106 (Keratinocytes) none | 0.0 | 0.0 | 0.0 |
| CCD1106 (Keratinocytes) | 0.0 | 0.0 | 0.0 |
| TNFalpha + IL-1beta | | | |
| Liver cirrhosis | 4.6 | 2.6 | 0.0 |
| NCI-H292 none | 0.0 | 1.7 | 0.0 |
| NCI-H292 IL-4 | 0.0 | 0.0 | 0.0 |
| NCI-H292 IL-9 | 0.0 | 0.7 | 0.0 |
| NCI-H292 IL-13 | 0.0 | 0.9 | 0.0 |
| NCI-H292 IFN gamma | 0.0 | 0.5 | 0.0 |
| HPAEC none | 0.0 | 0.0 | 0.0 |
| HPAEC TNF alpha + IL-1 beta | 0.0 | 0.0 | 0.0 |
| Lung fibroblast none | 31.4 | 95.9 | 0.2 |
| Lung fibroblast TNF | 22.2 | 48.6 | 0.1 |
| alpha + IL-1 beta | | | |
| Lung fibroblast IL-4 | 19.1 | 27.4 | 0.1 |
| Lung fibroblast IL-9 | 23.5 | 24.0 | 0.1 |
| Lung fibroblast IL-13 | 4.5 | 11. 9 | 0.0 |
| Lung fibroblast IFN gamma | 15.7 | 55.9 | 0.2 |
| Dermal fibroblast CCD1070 rest | 0.0 | 6.0 | 0.0 |
| Dermal fibroblast CCD1070 | 0.0 | 2.7 | 0.0 |
| TNF alpha | | | |
| Dermal fibroblast CCD1070 | 0.0 | 5.6 | 0.0 |
| IL-1 beta | | | |
| Dermal fibroblast IFN gamma | 8.5 | 30.6 | 0.1 |
| Dermal fibroblast IL-4 | 4.1 | 30.8 | 0.1 |
| Dermal Fibroblasts rest | 8.0 | 54.3 | 0.1 |
| Neutrophils TNFa + LPS | 0.0 | 0.9 | 0.0 |
| Neutrophils rest | 0.0 | 0.0 | 0.0 |
| Colon | 4.0 | 4.6 | 0.0 |
| Lung | 0.0 | 2.8 | 0.0 |
| Thymus | 0.0 | 0.0 | 0.0 |
| Kidney | 4.9 | 7.8 | 0.1 |

CNS_neurodegeneration_v1.0 Summary: Ag6428/Ag6430 This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

General_screening_panel_v1.6 Summary: Ag6424/Ag6425/Ag6428/Ag6430 Highest expression of this gene is detected in a ovarian cancer IGROV-1 cell line and brain cancer SNB-19 cell lines (CTs=25-31). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Panel 4.1D Summary Ag6425/Ag6428/Ag6430 Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Ag6424/Ag6432 Expression of this gene is low/undetectable (CTs>35) across all of the samples on this panel (data not shown).

AM. CG56054-04: Integrin Alpha 7-Like Protein.

Expression of gene CG56054-04 was assessed using the primer-probe sets Ag6424, Ag6427, Ag6430 and Ag6434, described in Tables AMA, AMB, AMC and AMD. Results of the RTQ-PCR runs are shown in Tables AME, AMF and AMG.

TABLE AMA

Probe Name Ag6424

| | | | | |
|---|---|---|---|---|
| Primers | Sequences | Length | Start Position | SEQ ID No |

| | | | | |
|---|---|---|---|---|
| Forward | 5′-ttgggttctgccagca-3′ | 16 | 742 | 459 |
| Probe | TET-5′-cacagctgccgccttctccc-3′-TAMRA | 20 | 761 | 460 |
| Reverse | 5′-aaaagcaaccccttccaa-3′ | 18 | 824 | 461 |

TABLE AMB

Probe Name Ag6427

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1394 | 462 |
| Probe | TET-5′-ccttcacaggtgctggagggc-3′-TAMRA | 21 | 1434 | 463 |
| Reverse | 5′-ccctggatgcccatc-3′ | 15 | 1484 | 464 |

TABLE AMC

Probe Name Ag6430

| | | | Start | |
|---|---|---|---|---|
| Primers | Sequences | Length | Position | SEQ ID No |
| Forward | 5′-gtgaccaacattgatagctcaga-3′ | 23 | 843 | 465 |
| Probe | TET-5′-ccccgaccagctggtgtataaaactttg-3′-TAMRA | 28 | 866 | 466 |
| Reverse | 5′-gggagccggtcagca-3′ | 15 | 899 | 467 |

TABLE AMD

Probe Name Ag6434

| | | | | |
|---|---|---|---|---|
| | | | Start | |
| Primers | Sequences | Length | Position | SEQ ID No |

| | | | | |
|---|---|---|---|---|
| Forward | 5′-cctttgatggtgatgggaa-3′ | 19 | 1372 | 468 |
| Probe | TET-5′-cttcatctaccatgggagcagcctg-3′-TAMRA | 25 | 1394 | 469 |
| Reverse | 5′-gctcgggatgcccac-3′ | 15 | 1461 | 470 |

TABLE AME
CNS_neurodegeneration_v1.0

| | Rel. Exp. (%) | Rel. Exp. (%) |
|---|---|---|
| | Ag6430, Run | Ag6434, Run |
| Tissue Name | 266937085 | 269253996 |
| AD 1 Hippo | 20.0 | 17.3 |
| AD 2 Hippo | 48.0 | 33.0 |
| AD 3 Hippo | 11.6 | 3.4 |
| AD 4 Hippo | 17.1 | 9.0 |
| AD 5 hippo | 39.2 | 66.4 |
| AD 6 Hippo | 100.0 | 100.0 |
| Control 2 Hippo | 17.9 | 23.3 |
| Control 4 Hippo | 38.4 | 26.6 |
| Control (Path) 3 Hippo | 10.2 | 7.0 |
| AD 1 Temporal Ctx | 12.1 | 13.7 |
| AD 2 Temporal Ctx | 36.6 | 35.8 |
| AD 3 Temporal Ctx | 11.7 | 7.2 |
| AD 4 Temporal Ctx | 15.6 | 6.7 |
| AD 5 Inf Temporal Ctx | 43.8 | 21.9 |
| AD 5 Sup Temporal Ctx | 56.6 | 31.6 |
| AD 6 Inf Temporal Ctx | 40.9 | 52.9 |
| AD 6 Sup Temporal Ctx | 44.1 | 71.2 |
| Control 1 Temporal Ctx | 11.9 | 10.3 |
| Control 2 Temporal Ctx | 16.7 | 16.2 |
| Control 3 Temporal Ctx | 13.0 | 8.5 |
| Control 4 Temporal Ctx | 18.9 | 13.6 |
| Control (Path) 1 Temporal Ctx | 32.5 | 29.9 |
| Control (Path) 2 Temporal Ctx | 19.5 | 13.2 |
| Control (Path) 3 Temporal Ctx | 12.9 | 9.2 |
| Control (Path) 4 Temporal Ctx | 19.8 | 13.8 |
| AD 1 Occipital Ctx | 16.2 | 8.4 |
| AD 2 Occipital Ctx (Missing) | 0.0 | 0.0 |
| AD 3 Occipital Ctx | 11.7 | 3.8 |
| AD 4 Occipital Ctx | 12.6 | 1.4 |
| AD 5 Occipital Ctx | 16.7 | 21.3 |
| AD 6 Occipital Ctx | 17.8 | 15.5 |
| Control 1 Occipital Ctx | 11.3 | 5.5 |
| Control 2 Occipital Ctx | 24.8 | 33.7 |
| Control 3 Occipital Ctx | 16.4 | 3.0 |
| Control 4 Occipital Ctx | 12.1 | 8.1 |
| Control (Path) 1 Occipital Ctx | 32.8 | 39.0 |
| Control (Path) 2 Occipital Ctx | 9.6 | 4.2 |
| Control (Path) 3 Occipital Ctx | 8.4 | 3.2 |
| Control (Path) 4 Occipital Ctx | 15.9 | 9.3 |
| Control 1 Parietal Ctx | 15.2 | 10.1 |
| Control 2 Parietal Ctx | 39.5 | 43.5 |
| Control 3 Parietal Ctx | 14.5 | 15.9 |
| Control (Path) 1 Parietal Ctx | 33.4 | 24.8 |
| Control (Path) 2 Parietal Ctx | 20.0 | 22.1 |
| Control (Path) 3 Parietal Ctx | 15.0 | 9.3 |
| Control (Path) 4 Parietal Ctx | 28.3 | 34.6 |

TABLE AMF
General_screening panel_v1.6

| | Rel. Exp. (%) | Rel. Exp. (%) | Rel. Exp. (%) | Rel. Exp. (%) |
|---|---|---|---|---|
| | Ag6424, Run | Ag6427, Run | Ag6430, Run | Ag6434, Run |
| Tissue Name | 277221719 | 277222437 | 277222443 | 277222451 |
| Adipose | 0.0 | 0.0 | 8.2 | 9.5 |
| Melanoma* Hs688(A).T | 0.0 | 0.0 | 0.5 | 0.9 |
| Melanoma* Hs688(B).T | 0.0 | 0.0 | 0.6 | 3.7 |
| Melanoma* M14 | 0.0 | 0.0 | 0.7 | 0.7 |
| Melanoma* LOXIMVI | 0.0 | 0.0 | 0.0 | 0.0 |
| Melanoma* SK-MEL-5 | 0.0 | 0.0 | 22.5 | 14.7 |
| Squamous cell carcinoma SCC-4 | 0.0 | 0.0 | 0.3 | 0.0 |
| Testis Pool | 0.0 | 0.0 | 4.2 | 5.7 |
| Prostate ca.* (bone met) PC-3 | 0.0 | 0.0 | 1.0 | 1.5 |
| Prostate Pool | 0.0 | 0.0 | 8.5 | 4.2 |
| Placenta | 0.0 | 0.0 | 0.1 | 0.5 |
| Uterus Pool | 0.0 | 0.0 | 2.6 | 2.5 |
| Ovarian ca. OVCAR-3 | 0.0 | 0.0 | 0.8 | 0.8 |
| Ovarian ca. SK-OV-3 | 0.0 | 0.0 | 1.5 | 0.8 |
| Ovarian ca. OVCAR-4 | 0.0 | 0.0 | 0.5 | 0.5 |
| Ovarian ca. OVCAR-5 | 0.0 | 0.0 | 1.5 | 2.9 |
| Ovarian ca. IGROV-1 | 100.0 | 100.0 | 90.8 | 73.7 |
| Ovarian ca. OVCAR-8 | 5.6 | 0.0 | 11.9 | 20.7 |
| Ovary | 0.0 | 0.0 | 2.1 | 4.0 |
| Breast ca. MCF-7 | 0.0 | 0.0 | 0.4 | 0.5 |
| Breast ca. MDA-MB-231 | 0.0 | 0.0 | 0.4 | 0.5 |
| Breast ca. BT 549 | 0.0 | 0.0 | 0.3 | 0.5 |
| Breast ca. T47D | 0.0 | 0.0 | 0.3 | 0.0 |
| Breast ca. MDA-N | 0.0 | 0.0 | 0.7 | 0.0 |
| Breast Pool | 0.0 | 0.0 | 19.5 | 9.6 |
| Trachea | 0.0 | 0.0 | 2.9 | 5.3 |
| Lung | 0.0 | 0.0 | 1.3 | 1.3 |
| Fetal Lung | 0.0 | 0.0 | 4.0 | 5.0 |
| Lung ca. NCI-N417 | 2.0 | 0.0 | 2.7 | 3.0 |
| Lung ca. LX-1 | 3.1 | 0.0 | 7.0 | 4.3 |
| Lung ca. NCI-H146 | 0.0 | 0.0 | 0.5 | 0.0 |
| Lung ca. SHP-77 | 2.3 | 0.0 | 6.3 | 4.9 |
| Lung ca. A549 | 0.0 | 0.0 | 0.3 | 0.7 |
| Lung ca. NCI-H526 | 0.0 | 0.0 | 0.7 | 0.0 |
| Lung ca. NCI-H23 | 0.0 | 0.0 | 4.5 | 3.1 |
| Lung ca. NCI-H460 | 0.0 | 0.0 | 0.2 | 0.0 |
| Lung ca. HOP-62 | 0.0 | 0.0 | 0.6 | 0.0 |
| Lung ca. NCI-H522 | 0.0 | 0.0 | 2.4 | 1.4 |
| Liver | 0.0 | 0.0 | 0.1 | 0.0 |
| Fetal Liver | 0.0 | 0.0 | 0.6 | 0.5 |
| Liver ca. HepG2 | 0.0 | 0.0 | 0.1 | 0.5 |
| Kidney Pool | 6.5 | 0.0 | 34.9 | 22.8 |
| Fetal Kidney | 0.0 | 0.0 | 5.1 | 2.4 |
| Renal ca. 786-0 | 0.0 | 0.0 | 0.2 | 0.0 |
| Renal ca. A498 | 0.0 | 0.0 | 0.1 | 0.0 |
| Renal ca. ACHN | 0.0 | 0.0 | 0.7 | 0.7 |
| Renal ca. UO-31 | 0.0 | 0.0 | 0.3 | 0.0 |
| Renal ca. TK-10 | 0.0 | 0.0 | 2.5 | 3.0 |
| Bladder | 0.0 | 0.0 | 3.0 | 3.4 |
| Gastric ca. (liver met.) NCI-N87 | 0.0 | 0.0 | 1.7 | 1.1 |
| Gastric ca. KATO III | 0.0 | 0.0 | 0.4 | 0.0 |
| Colon ca. SW-948 | 0.0 | 0.0 | 0.0 | 0.0 |
| Colon ca. SW480 | 9.5 | 0.0 | 39.0 | 28.3 |
| Colon ca.* (SW480 met) SW620 | 7.7 | 0.0 | 15.5 | 11.7 |
| Colon ca. HT29 | 0.0 | 0.0 | 0.0 | 0.0 |
| Colon ca. HCT-116 | 1.6 | 0.0 | 3.8 | 5.0 |
| Colon ca. CaCo-2 | 10.4 | 0.0 | 22.2 | 14.9 |
| Colon cancer tissue | 0.0 | 0.0 | 6.5 | 9.2 |
| Colon ca. SW1116 | 0.0 | 0.0 | 1.7 | 2.2 |
| Colon ca. Colo-205 | 0.0 | 0.0 | 0.2 | 0.0 |
| Colon ca. SW-48 | 0.0 | 0.0 | 1.3 | 1.4 |
| Colon Pool | 0.0 | 0.0 | 28.7 | 14.2 |
| Small Intestine Pool | 0.0 | 0.0 | 10.5 | 7.4 |
| Stomach Pool | 0.0 | 0.0 | 6.2 | 9.2 |
| Bone Marrow Pool | 0.0 | 0.0 | 11.3 | 4.6 |
| Fetal Heart | 0.0 | 0.0 | 24.3 | 11.3 |
| Heart Pool | 5.2 | 0.0 | 23.0 | 15.2 |
| Lymph Node Pool | 0.0 | 0.0 | 30.4 | 14.1 |
| Fetal Skeletal Muscle | 36.9 | 0.0 | 46.7 | 33.0 |
| Skeletal Muscle Pool | 12.3 | 0.0 | 21.5 | 21.2 |
| Spleen Pool | 0.0 | 0.0 | 2.0 | 1.2 |
| Thymus Pool | 0.0 | 0.0 | 7.5 | 6.1 |
| CNS cancer (glio/astro) U87-MG | 1.6 | 0.0 | 6.1 | 10.4 |
| CNS cancer (glio/astro) U-118-MG | 0.0 | 0.0 | 2.9 | 3.4 |
| CNS cancer (neuro; met) SK-N-AS | 0.0 | 0.0 | 1.7 | 1.8 |
| CNS cancer (astro) SF-539 | 0.0 | 0.0 | 0.2 | 0.0 |
| CNS cancer (astro) SNB-75 | 1.9 | 0.0 | 5.9 | 12.0 |
| CNS cancer (glio) SNB-19 | 84.1 | 25.0 | 100.0 | 100.0 |
| CNS cancer (glio) SF-295 | 1.8 | 0.0 | 9.0 | 7.7 |
| Brain (Amygdala) Pool | 2.3 | 10.0 | 6.9 | 5.5 |
| Brain (cerebellum) | 6.6 | 0.0 | 11.1 | 11.0 |
| Brain (fetal) | 3.0 | 0.0 | 11.5 | 6.9 |
| Brain (Hippocampus) Pool | 3.1 | 0.0 | 11.0 | 8.5 |
| Cerebral Cortex Pool | 1.7 | 0.0 | 7.5 | 6.8 |
| Brain (Substantia nigra) Pool | 1.8 | 0.0 | 8.5 | 5.2 |
| Brain (Thalamus) Pool | 0.0 | 0.0 | 10.0 | 6.8 |
| Brain (whole) | 0.0 | 0.0 | 8.0 | 6.8 |
| Spinal Cord Pool | 3.2 | 0.0 | 12.8 | 6.4 |
| Adrenal Gland | 0.0 | 0.0 | 6.1 | 8.4 |
| Pituitary gland Pool | 0.0 | 0.0 | 0.8 | 0.6 |
| Salivary Gland | 0.0 | 0.0 | 1.1 | 1.6 |
| Thyroid (female) | 0.0 | 0.0 | 0.8 | 2.6 |
| Pancreatic ca. CAPAN2 | 0.0 | 0.0 | 0.8 | 0.9 |
| Pancreas Pool | 0.0 | 0.0 | 1.1 | 0.8 |

TABLE AMG
Panel 4.1D

| | Rel.Exp. (%) | Rel.Exp. (%) |
|---|---|---|
| | Ag6430, Run | Ag6434, Run |
| Tissue Name | 268767563 | 268713326 |
| Secondary Th1 act | 0.0 | 0.0 |
| Secondary Th2 act | 0.0 | 0.0 |
| Secondary Tr1 act | 0.0 | 0.0 |
| Secondary Th1 rest | 0.0 | 0.0 |
| Secondary Th2 rest | 0.0 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 0.0 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 | 0.0 | 0.0 |
| lymphocyte act | | |
| CD45RO CD4 | 0.0 | 3.9 |
| lymphocyte act | | |
| CD8 lymphocyte act | 0.0 | 0.0 |
| Secondary CD8 | 0.0 | 0.0 |
| lymphocyte rest | | |
| Secondary CD8 | 0.0 | 0.0 |
| lymphocyte act | | |
| CD4 lymphocyte none | 0.0 | 0.0 |
| 2ry Th1/Th2/Tr1 | 0.0 | 0.0 |
| anti-CD95 CH11 | | |
| LAK cells rest | 0.1 | 7.9 |
| LAK cells IL-2 | 0.0 | 0.0 |
| LAK cells IL-2 + IL-12 | 0.0 | 0.0 |
| LAK cells IL-2 + IFN | 0.0 | 0.0 |
| gamma | | |
| LAK cells IL-2 + IL-18 | 0.0 | 0.0 |
| LAK cells PMA/ionomycin | 0.1 | 7.0 |
| NK Cells IL-2 rest | 0.0 | 0.0 |
| Two Way MLR 3 day | 0.0 | 0.0 |
| Two Way MLR 5 day | 0.0 | 0.0 |
| Two Way MLR 7 day | 0.0 | 0.0 |
| PBMC rest | 0.0 | 0.0 |
| PBMC PWM | 0.0 | 0.0 |
| PBMC PHA-L | 0.0 | 0.0 |
| Ramos (B cell) none | 0.0 | 0.0 |
| Ramos (B cell) | 0.0 | 0.0 |
| ionomycin | | |
| B lymphocytes PWM | 0.0 | 0.0 |
| B lymphocytes | 0.0 | 0.0 |
| CD40L and IL-4 | | |
| EOL-1 dbcAMP | 0.1 | 4.4 |
| EOL-1 dbcAMP | 0.0 | 0.0 |
| PMA/ionomycin | | |
| Dendritic cells none | 0.0 | 4.5 |
| Dendritic cells LPS | 0.0 | 0.0 |
| Dendritic cells anti-CD40 | 0.0 | 0.0 |
| Monocytes rest | 0.0 | 0.0 |
| Monocytes LPS | 0.0 | 5.9 |
| Macrophages rest | 0.0 | 0.0 |
| Macrophages LPS | 0.1 | 9.1 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + | 0.0 | 0.0 |
| IFN gamma | | |
| HUVEC TNF alpha + IL4 | 0.0 | 0.0 |
| HUVEC IL-11 | 0.0 | 0.0 |
| Lung Microvascular | 0.0 | 0.0 |
| EC none | | |
| Lung Microvascular | 0.0 | 0.0 |
| EC TNFalpha + IL- | | |
| 1beta | | |
| Microvascular Dermal | 0.0 | 0.0 |
| EC none | | |
| Microsvasular Dermal | 0.0 | 0.0 |
| EC TNFalpha + IL- | | |
| 1beta | | |
| Bronchial epithelium | 0.0 | 0.0 |
| TNFalpha + IL1beta | | |
| Small airway | 0.0 | 0.0 |
| epithelium none | | |
| Small airway epithelium | 0.0 | 0.0 |
| TNFalpha + IL-1beta | | |
| Coronery artery SMC rest | 0.0 | 0.0 |
| Coronery artery SMC | 0.0 | 0.0 |
| TNFalpha + IL-1beta | | |
| Astrocytes rest | 12.0 | 100.0 |
| Astrocytes TNFalpha + | 100.0 | 97.3 |
| IL-1beta | | |
| KU-812 (Basophil) rest | 0.0 | 0.0 |
| KU-812 (Basophil) | 0.0 | 0.0 |
| PMA/ionomycin | | |
| CCD1106 | 0.0 | 0.0 |
| (Keratinocytes) none | | |
| CCD1106 | 0.0 | 0.0 |
| (Keratinocytes) | | |
| TNFalpha + IL-1beta | | |
| Liver cirrhosis | 0.0 | 3.4 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + | 0.0 | 0.0 |
| IL-1 beta | | |
| Lung fibroblast none | 0.2 | 72.7 |
| Lung fibroblast TNF | 0.1 | 36.6 |
| alpha + IL-1 beta | | |
| Lung fibroblast IL-4 | 0.1 | 62.4 |
| Lung fibroblast IL-9 | 0.1 | 52.5 |
| Lung fibroblast IL-13 | 0.0 | 14.6 |
| Lung fibroblast IFN | 0.2 | 41.5 |
| gamma | | |
| Dermal fibroblast | 0.0 | 5.1 |
| CCD1070 rest | | |
| Dermal fibroblast | 0.0 | 7.2 |
| CCD1070 TNF alpha | | |
| Dermal fibroblast | 0.0 | 0.0 |
| CCD1070 IL-1 beta | | |
| Dermal fibroblast IFN | 0.1 | 24.5 |
| gamma | | |
| Dermal fibroblast IL-4 | 0.1 | 28.7 |
| Dermal Fibroblasts rest | 0.1 | 44.4 |
| Neutrophils | 0.0 | 0.0 |
| TNFa + LPS | | |
| Neutrophils rest | 0.0 | 0.0 |
| Colon | 0.0 | 4.1 |
| Lung | 0.0 | 0.0 |
| Thymus | 0.0 | 0.0 |
| Kidney | 0.1 | 8.1 |

CNS_neurodegeneration_v1.0 Summary: Ag6430/Ag6434 This panel confirms the expression of this gene at low levels in the brains of an independent group of individuals. However, no differential expression of this gene was detected between Alzheimer's diseased postmortem brains and those of non-demented controls in this experiment. See Panel 1.6 for a discussion of this gene in treatment of central nervous system disorders.

General_screening_panel_v1.6 Summary: Ag6424/Ag6430/Ag6434 Highest expression of this gene is detected in a ovarian cancer IGROV-1 cell line and brain cancer SNB-19 cell lines (CTs=25-33.7). In addition, moderate to low levels of expression of this gene is also seen in some of the colon, ovarian and brain cancer cell lines. Thus, expression of this gene may be used as a marker to detect the presence of colon, ovarian and brain cancers. Furthermore, therapeutic modulation of this gene may be useful in the treatment of these cancers. Moderate to low levels of expression of this gene is also seen in all the regions of central nervous system, tissues with metabolic/endocrine functions, and number of cancer cell lines. Moderate levels of expression of this gene is seen in normal tissues represented by breast, testis, prostate, uterus, gastrointestinal tract, and tissues with metabolic/endocrine functions including adipose, heart, skeletal muscle, and adernal gland. Therefore, therapeutic modulation of this gene or its protein product may be useful in the treatment of diseases associated with these tissues, including obesity, diabetes and inflammatory bowel disease. In addition, moderate to low levels of expression of this gene is also seen in some regions of central nervous system, and some brain, colon and ovarian cancer cell lines.

Panel 4.1D Summary: Ag6430/Ag6434 Highest expression of this gene is detected in both resting and cytokine activated astrocytes (CTs=22-34.8). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AN. CG56054-05: Integrin Alpha 7-Like Protein.

Expression of gene CG56054-05 was assessed using the primer-probe set Ag6436, described in Table ANA.

TABLE ANA

Probe Name Ag6436

| | | | | |
|---|---|---|---|---|
| Primers | Sequences | Length | Start Position | SEQ ID No |

| | | | | |
|---|---|---|---|---|
| Forward | 5′-gggcaagattgttacctgtg-3′ | 20 | 402 | 471 |
| Probe | TET-5′-ctgacgggcatcccgagct-3′-TAMRA | 19 | 440 | 472 |
| Reverse | 5′-ccctggatgcccatc-3′ | 15 | 466 | 473 |

AO. CG56054-06 and CG56054-07: Integrin Alpha 7-Like Protein.

Expression of gene CG56054-06 and CG56054-07 was assessed using the primer-probe sets Ag4983, Ag6442, Ag6425, Ag6431, Ag6438, Ag6439, Ag6440, Ag6413 and Ag6964, described in Tables AOA, AOB, AOC, AOD, AOE, AOF, AOG, AOH and AOI. Results of the RTQ-PCR runs are shown in Tables AOJ, AOK, AOL, AOM, AON and AOO. Note that CG56054-07 is recognized by probe-primer sets Ag6425 and Ag6440.

TABLE AOA

Probe Name Ag4983

| | | | Start | |
|---|---|---|---|---|
| Primers | Sequences | Length | Position | SEQ ID No |
| Forward | 5′-ccaggtcaccttctacctcatc-3′ | 22 | 1057 | 474 |
| Probe | TET-5′-cttagcacctccgggatcagcatt-3′-TAMRA | 24 | 1079 | 475 |
| Reverse | 5′-aacagcagctctacctccagtt-3′ | 22 | 1113 | 476 |

TABLE AOB

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3′ | 22 | 1496 | 477 |
| Probe | TET-5′-ccacctgagcagcaggagcct-3′-TAMRA | 21 | 1535 | 478 |
| Reverse | 5′-gcgcagtccagggtg-3′ | 15 | 1621 | 479 |

TABLE AOC

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 2118 | 480 |
| Probe | TET-5′-catcccgagctgggcccc-3′-TAMRA | 18 | 2150 | 481 |
| Reverse | 5′-gccctggatgcccat-3′ | 15 | 2169 | 482 |

TABLE AOD

Probe Name Ag6431

| | | | Start | |
|---|---|---|---|---|
| Primers | Sequences | Length | Position | SEQ ID No |
| Forward | 5′-aaacatcaccctggactgc-3′ | 19 | 1615 | 483 |
| Probe | TET-5′-tggtgttcagctgcccactctacag-3′-TAMRA | 25 | 1656 | 484 |
| Reverse | 5′-ccgcgcggtcaaa-3′ | 13 | 1682 | 485 |

TABLE AOE

Probe Name Ag6438

| | | | | |
|---|---|---|---|---|
| Primers | Sequences | Length | Start Position | SEQ ID No |

| | | | | |
|---|---|---|---|---|
| Forward | 5′-cagttgcagccctgga-3′ | 16 | 342 | 486 |
| Probe | TET-5′-ccaggttccccgtgtgacgttc-3′-TAMRA | 22 | 397 | 487 |
| Reverse | 5′-tcttccaggttacggctca-3′ | 19 | 420 | 488 |

TABLE AOF

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 1872 | 489 |
| Probe | TET-5′-ccctggtgggtcatcctcctg-3′-TAMRA | 21 | 1892 490 | |
| Reverse | 5′-gaagaatcccatcttccacag-3′ | 21 | 1958 | 491 |

TABLE AOG

Probe Name Ag6440

| | | | | |
|---|---|---|---|---|
| Primers | Sequences | Length | Start Position | SEQ ID No |

| | | | | |
|---|---|---|---|---|
| Forward | 5′-accatcctgaggaacaactg-3′ | 20 | 2075 | 492 |
| Probe | ctgacgggcatcccgagct-3′-TAMRA | 19 | 2142 | 493 |
| Reverse | 5′-ccctggatqcccatc-3′ | 15 | 2168 | 494 |

TABLE AOH

Probe Name Ag6413

| | | | | |
|---|---|---|---|---|
| Primers | Sequences | Length | Start Position | SEQ ID No |

| | | | | |
|---|---|---|---|---|
| Forward | 5′-ggtgaagacaagatctgccag-3′ | 21 | 695 | 495 |
| Probe | TET-5′-tgtacccgggtcagcgacacg-3′-TAMRA | 21 | 746 | 496 |
| Reverse | 5′-gctgttgttccatccacatc-3-40 | 20 | 788 | 497 |

TABLE AOI

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatgca-3′ | 15 | 1701 | 498 |
| Probe | TET-5′-actctacagctttgaccgcqcgg-3′-TAMRA | 23 | 1672 | 499 |
| Reverse | 5′-gccaactgtgtggtgttca-3′ | 19 | 1646 | 500 |

TABLE AOJ
CNS_neurodegeneration_v1.0

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag4983, | Ag6413, | Ag6431, | Ag6439, | Ag6440, | Ag6442, |
| | Run | Run | Run | Run | Run | Run |
| Tissue Name | 218649223 | 269253983 | 268030722 | 269254002 | 269254003 | 264979298 |
| AD 1 Hippo | 23.7 | 24.8 | 18.8 | 21.6 | 18.9 | 19.2 |
| AD 2 Hippo | 41.2 | 52.9 | 28.7 | 28.9 | 61.1 | 49.7 |
| AD 3 Hippo | 8.9 | 6.4 | 7.5 | 6.1 | 9.7 | 20.4 |
| AD 4 Hippo | 14.8 | 25.5 | 18.8 | 17.6 | 23.3 | 5.6 |
| AD 5 Hippo | 44.8 | 41.8 | 38.4 | 42.6 | 34.6 | 57.4 |
| AD 6 Hippo | 100.0 | 100.0 | 100.0 | 100.0 | 100.0 | 90.1 |
| Control 2 | 24.3 | 36.1 | 29.5 | 32.5 | 29.9 | 28.5 |
| Hippo | | | | | | |
| Control 4 | 42.9 | 43.8 | 32.3 | 37.9 | 54.7 | 86.5 |
| Hippo | | | | | | |
| Control (Path) | 14.2 | 11.4 | 6.0 | 6.4 | 5.8 | 0.0 |
| 3 Hippo | | | | | | |
| AD 1 | 23.3 | 15.9 | 17.1 | 24.5 | 12.6 | 16.8 |
| Temporal Ctx | | | | | | |
| AD 2 | 41.5 | 47.3 | 39.8 | 27.5 | 59.0 | 21.6 |
| Temporal Ctx | | | | | | |
| AD 3 | 9.5 | 9.8 | 11.3 | 9.0 | 17.1 | 5.7 |
| Temporal Ctx | | | | | | |
| AD 4 | 30.6 | 39.0 | 25.3 | 30.4 | 29.9 | 8.7 |
| Temporal Ctx | | | | | | |
| AD 5 Inf | 45.4 | 37.1 | 36.3 | 41.8 | 41.8 | 73.7 |
| Temporal Ctx | | | | | | |
| AD 5 Sup | 51.1 | 39.0 | 32.3 | 38.7 | 39.2 | 55.9 |
| Temporal Ctx | | | | | | |
| AD 6 Inf | 38.2 | 59.9 | 46.7 | 47.6 | 48.6 | 76.8 |
| Temporal Ctx | | | | | | |
| AD 6 Sup | 43.8 | 48.6 | 50.3 | 50.3 | 17.0 | 59.9 |
| Temporal Ctx | | | | | | |
| Control 1 | 12.2 | 23.0 | 15.6 | 24.0 | 23.3 | 46.7 |
| Temporal Ctx | | | | | | |
| Control 2 | 14.2 | 32.5 | 17.4 | 14.9 | 43.5 | 50.0 |
| Temporal Ctx | | | | | | |
| Control 3 | 15.1 | 15.3 | 14.5 | 16.5 | 9.2 | 9.5 |
| Temporal Ctx | | | | | | |
| Control 3 | 23.7 | 25.0 | 13.1 | 23.8 | 30.1 | 13.6 |
| Temporal Ctx | | | | | | |
| Control (Path) | 26.1 | 47.0 | 30.6 | 39.8 | 51.1 | 46.0 |
| 1 Temporal | | | | | | |
| Ctx | | | | | | |
| Control (Path) | 24.5 | 25.9 | 20.4 | 24.8 | 7.2 | 0.0 |
| 2 Temporal | | | | | | |
| Ctx | | | | | | |
| Control (Path) | 11.7 | 16.0 | 10.9 | 11.9 | 9.9 | 31.0 |
| 3 Temporal | | | | | | |
| Ctx | | | | | | |
| Control (Path) | 21.9 | 27.4 | 18.2 | 21.6 | 14.9 | 39.5 |
| 4 Temporal | | | | | | |
| Ctx | | | | | | |
| AD 1 Occipital | 16.0 | 11.9 | 11.5 | 16.0 | 5.8 | 6.3 |
| CTX | | | | | | |
| AD 2 Occipital | 0.0 | 0.0 | 0.0 | 0.0 | 0.0 | 0.0 |
| Ctx (Missing) | | | | | | |
| AD 3 Occipital | 10.7 | 6.0 | 8.8 | 10.2 | 7.8 | 4.9 |
| Ctx | | | | | | |
| AD 4 Occipital | 18.9 | 23.7 | 17.9 | 18.6 | 35.4 | 11.1 |
| Ctx | | | | | | |
| AD 5 Occipital | 24.8 | 28.3 | 22.5 | 22.7 | 16.6 | 42.3 |
| Ctx | | | | | | |
| AD 6 Occipital | 20.6 | 31.9 | 17.0 | 22.1 | 23.5 | 14.8 |
| Ctx | | | | | | |
| Control 1 | 9.5 | 14.4 | 8.7 | 7.2 | 15.2 | 8.8 |
| Occipital Ctx | | | | | | |
| Control 2 | 31.9 | 42.6 | 33.2 | 29.3 | 35.8 | 82.4 |
| Occipital Ctx | | | | | | |
| Control 3 | 18.8 | 13.0 | 17.1 | 19.2 | 4.4 | 8.8 |
| Occipital Ctx | | | | | | |
| Control 4 | 18.2 | 17.0 | 12.6 | 13.6 | 12.9 | 24.0 |
| Occipital Ctx | | | | | | |
| Control (Path) | 38.2 | 52.5 | 36.1 | 39.5 | 22.4 | 100.0 |
| 1 Occipital Ctx | | | | | | |
| Control (Path) | 9.6 | 14.1 | 7.9 | 7.0 | 5.0 | 9.3 |
| 2 Occipital Ctx | | | | | | |
| Control (Path) | 4.8 | 8.7 | 6.0 | 5.9 | 6.7 | 4.1 |
| 3 Occipital Ctx | | | | | | |
| Control (Path) | 16.2 | 13.2 | 10.2 | 11.4 | 11.9 | 32.8 |
| 4 Occipital Ctx | | | | | | |
| Control 1 | 14.4 | 21.9 | 16.3 | 15.7 | 33.2 | 9.2 |
| Parietal Ctx | | | | | | |
| Control 2 | 32.8 | 28.9 | 28.3 | 37.1 | 17.4 | 28.1 |
| Parietal Ctx | | | | | | |
| Control 3 | 20.6 | 19.8 | 8.7 | 10.8 | 21.6 | 9.1 |
| Parietal Ctx | | | | | | |
| Control (Path) | 35.4 | 62.4 | 39.2 | 37.9 | 47.3 | 69.3 |
| 1 Parietal Ctx | | | | | | |
| Control (Path) | 22.1 | 23.8 | 22.5 | 18.7 | 17.1 | 37.6 |
| 2 Parietal Ctx | | | | | | |
| Control (Path) | 11.2 | 15.4 | 7.1 | 12.0 | 11.7 | 10.4 |
| 3 Parietal Ctx | | | | | | |
| Control (Path) | 31.2 | 34.2 | 8.8 | 27.9 | 29.3 | 27.5 |
| 4 Parietal Ctx | | | | | | |

TABLE AOK
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 13.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro; met) SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE AOL
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro, met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE AOM
General_screening_panel_v1.6

| | Rel. | Rel. | Rel. | Rel. | Rel. | Rel. | Rel. | Rel. |
|---|---|---|---|---|---|---|---|---|
| | Exp. (%) | Exp. (%) | Exp. (%) | Exp. (%) | Exp. (%) | Exp. (%) | Exp. (%) | Exp. (%) |
| | Ag6413, | Ag6425, | Ag6431, | Ag6431, | Ag6438, | Ag6439, | Ag6440, | Ag6964, |
| Tissue | Run | Run | Run | Run | Run | Run | Run | Run |
| Name | 277249371 | 277221721 | 277633568 | 278389390 | 277223173 | 277223175 | 277223177 | 278388946 |
| Adipose | 25.9 | 2.6 | 17.4 | 13.8 | 25.0 | 17.3 | 3.7 | 18.8 |
| Melanoma* | 0.5 | 0.0 | 0.8 | 0.9 | 0.0 | 0.4 | 0.0 | 0.7 |
| Hs688(A).T | | | | | | | | |
| Melanoma* | 2.7 | 0.2 | 2.5 | 2.2 | 0.0 | 2.9 | 0.8 | 2.4 |
| Hs688(B).T | | | | | | | | |
| Melanoma* | 0.3 | 0.0 | 0.4 | 0.4 | 0.0 | 0.4 | 0.0 | 0.7 |
| M14 | | | | | | | | |
| Melanoma* | 0.0 | 0.0 | 0.0 | 0.0 | 0.0 | 0.0 | 0.0 | 0.1 |
| LOXIMVI | | | | | | | | |
| Melanoma* | 15.2 | 2.2 | 18.2 | 14.6 | 29.5 | 18.3 | 3.0 | 15.9 |
| SK-MEL-5 | | | | | | | | |
| Squamous | 0.0 | 0.0 | 0.1 | 0.2 | 0.0 | 0.0 | 0.0 | 0.1 |
| cell | | | | | | | | |
| carcinoma | | | | | | | | |
| SCC-4 | | | | | | | | |
| Testis | 5.2 | 3.5 | 10.4 | 9.0 | 4.6 | 9.1 | 3.0 | 9.9 |
| Pool | | | | | | | | |
| Prostate | 1.9 | 0.5 | 1.9 | 1.8 | 0.0 | 1.3 | 1.2 | 4.3 |
| ca.* (bone | | | | | | | | |
| met) PC-3 | | | | | | | | |
| Prostate | 8.1 | 1.0 | 11.3 | 12.1 | 7.7 | 28.5 | 2.1 | 10.0 |
| Pool | | | | | | | | |
| Placenta | 0.5 | 0.0 | 0.1 | 0.1 | 0.0 | 0.5 | 0.0 | 0.4 |
| Uterus | 2.2 | 1.5 | 4.6 | 4.5 | 0.0 | 5.3 | 2.3 | 4.1 |
| Pool | | | | | | | | |
| Ovarian | 0.9 | 0.3 | 0.7 | 1.1 | 0.0 | 1.6 | 0.4 | 4.0 |
| ca. | | | | | | | | |
| OVCAR-3 | | | | | | | | |
| Ovarian | 0.8 | 0.2 | 0.8 | 0.9 | 0.0 | 1.3 | 0.5 | 1.7 |
| ca. SK- | | | | | | | | |
| OV-3 | | | | | | | | |
| Ovarian | 0.2 | 0.0 | 0.4 | 0.8 | 0.0 | 0.9 | 0.0 | 0.5 |
| ca. | | | | | | | | |
| OVCAR-4 | | | | | | | | |
| Ovarian | 1.6 | 1.3 | 1.3 | 1.7 | 0.0 | 1.4 | 4.2 | 7.9 |
| ca. | | | | | | | | |
| OVCAR-5 | | | | | | | | |
| Ovarian | 100.0 | 100.0 | 84.7 | 97.9 | 3.5 | 69.3 | 100.0 | 75.8 |
| ca. | | | | | | | | |
| IGROV-1 | | | | | | | | |
| Ovarian | 13.6 | 21.9 | 15.6 | 14.6 | 0.0 | 17.3 | 18.2 | 16.7 |
| ca. | | | | | | | | |
| OVCAR-8 | | | | | | | | |
| Ovary | 2.7 | 0.3 | 3.1 | 2.3 | 0.8 | 2.8 | 0.8 | 2.4 |
| Breast ca. | 0.3 | 0.0 | 0.1 | 0.2 | 0.0 | 0.5 | 0.3 | 0.5 |
| MCF-7 | | | | | | | | |
| Breast ca. | 0.1 | 0.0 | 0.2 | 0.2 | 0.0 | 0.2 | 0.0 | 0.3 |
| MDA- | | | | | | | | |
| MB-231 | | | | | | | | |
| Breast ca. | 0.5 | 0.0 | 0.1 | 0.5 | 0.0 | 0.6 | 0.0 | 0.4 |
| BT 549 | | | | | | | | |
| Breast ca. | 0.0 | 0.0 | 0.2 | 0.3 | 0.0 | 0.4 | 0.3 | 0.5 |
| T47D | | | | | | | | |
| Breast ca. | 0.6 | 0.0 | 0.6 | 0.6 | 0.0 | 0.6 | 0.3 | 0.8 |
| MDA-N | | | | | | | | |
| Breast | 15.0 | 4.1 | 14.6 | 10.7 | 21.9 | 12.2 | 3.5 | 16.7 |
| Pool | | | | | | | | |
| Trachea | 4.5 | 0.7 | 4.8 | 4.2 | 9.7 | 4.7 | 1.4 | 5.6 |
| Lung | 2.8 | 0.7 | 4.2 | 3.2 | 0.0 | 3.9 | 5.3 | 5.1 |
| Fetal Lung | 3.9 | 0.3 | 5.0 | 4.8 | 7.4 | 5.3 | 2.9 | 6.1 |
| Lung ca. | 2.0 | 0.9 | 3.3 | 2.6 | 0.0 | 4.0 | 2.0 | 2.3 |
| NCI-N417 | | | | | | | | |
| Lung ca. | 3.5 | 2.7 | 5.0 | 3.5 | 0.0 | 4.9 | 6.3 | 44.1 |
| LX-1 | | | | | | | | |
| Lung ca. | 0.1 | 0.0 | 0.1 | 0.2 | 0.0 | 0.1 | 0.0 | 0.1 |
| NCI-H146 | | | | | | | | |
| Lung ca. | 4.0 | 0.4 | 5.3 | 4.5 | 0.0 | 4.5 | 0.8 | 3.8 |
| SHP-77 | | | | | | | | |
| Lung ca. | 0.3 | 2.6 | 0.0 | 0.4 | 0.0 | 0.6 | 2.2 | 4.7 |
| A549 | | | | | | | | |
| Lung ca. | 0.2 | 0.0 | 0.6 | 0.3 | 0.0 | 0.4 | 0.3 | 0.5 |
| NCI-H526 | | | | | | | | |
| Lung ca. | 2.9 | 1.0 | 4.8 | 3.2 | 0.0 | 2.9 | 2.3 | 10.3 |
| NCI-H23 | | | | | | | | |
| Lung ca. | 0.0 | 0.0 | 0.1 | 0.3 | 0.0 | 0.0 | 0.0 | 0.3 |
| NCI-H460 | | | | | | | | |
| Lung ca. | 0.5 | 0.0 | 1.0 | 0.6 | 0.0 | 0.5 | 0.0 | 0.7 |
| HOP-62 | | | | | | | | |
| Lung ca. | 1.7 | 0.6 | 1.7 | 1.3 | 0.0 | 3.3 | 2.5 | 8.9 |
| NCI-H522 | | | | | | | | |
| Liver | 0.1 | 0.0 | 0.0 | 0.0 | 0.0 | 0.1 | 0.4 | 2.0 |
| Fetal Liver | 0.3 | 0.3 | 0.6 | 0.5 | 0.0 | 0.8 | 0.8 | 8.2 |
| Liver ca. | 0.1 | 0.3 | 0.0 | 0.2 | 0.0 | 0.1 | 0.9 | 2.4 |
| HepG2 | | | | | | | | |
| Kidney | 27.9 | 0.0 | 33.9 | 28.1 | 100.0 | 43.2 | 14.6 | 32.8 |
| Pool | | | | | | | | |
| Fetal | 1.4 | 0.0 | 4.1 | 4.0 | 2.4 | 5.8 | 3.4 | 11.5 |
| Kidney | | | | | | | | |
| Renal ca. | 0.2 | 0.0 | 0.3 | 0.1 | 0.0 | 0.3 | 0.0 | 0.9 |
| 786-0 | | | | | | | | |
| Renal ca. | 0.0 | 1.8 | 0.0 | 0.3 | 0.0 | 0.5 | 3.8 | 8.5 |
| A498 | | | | | | | | |
| Renal ca. | 1.5 | 0.5 | 1.7 | 1.5 | 0.9 | 1.2 | 0.5 | 2.5 |
| ACHN | | | | | | | | |
| Renal ca. | 0.3 | 0.0 | 0.2 | 0.2 | 0.0 | 0.6 | 0.0 | 0.3 |
| UO-31 | | | | | | | | |
| Renal ca. | 1.9 | 0.4 | 2.0 | 1.9 | 0.0 | 2.1 | 0.5 | 4.6 |
| TK-10 | | | | | | | | |
| Bladder | 4.2 | 0.0 | 5.5 | 5.1 | 6.1 | 8.3 | 0.9 | 6.7 |
| Gastric ca. | 0.9 | 0.0 | 0.9 | 1.2 | 0.0 | 1.1 | 0.8 | 6.7 |
| (liver | | | | | | | | |
| met.) NCI- | | | | | | | | |
| N87 | | | | | | | | |
| Gastric ca. | 0.4 | 0.5 | 0.2 | 0.3 | 0.0 | 0.4 | 0.4 | 0.9 |
| KATO III | | | | | | | | |
| Colon ca. | 0.0 | 1.5 | 0.2 | 0.2 | 0.0 | 0.3 | 2.2 | 1.2 |
| SW-948 | | | | | | | | |
| Colon ca. | 20.9 | 5.2 | 27.0 | 23.3 | 42.3 | 23.0 | 6.3 | 33.7 |
| SW480 | | | | | | | | |
| Colon ca.* | 13.3 | 4.8 | 12.8 | 10.3 | 8.8 | 6.1 | 7.2 | 25.0 |
| (SW480 | | | | | | | | |
| met) | | | | | | | | |
| SW620 | | | | | | | | |
| Colon ca. | 0.2 | 0.0 | 0.2 | 0.2 | 0.0 | 0.0 | 0.3 | 0.3 |
| HT29 | | | | | | | | |
| Colon ca. | 2.1 | 0.2 | 2.5 | 2.0 | 0.0 | 2.1 | 0.6 | 4.3 |
| HCT-116 | | | | | | | | |
| Colon ca. | 15.0 | 3.6 | 19.1 | 16.7 | 31.4 | 18.3 | 6.5 | 38.2 |
| CaCo-2 | | | | | | | | |
| Colon | 9.0 | 3.3 | 11.9 | 7.6 | 6.3 | 7.7 | 4.4 | 20.4 |
| cancer | | | | | | | | |
| tissue | | | | | | | | |
| Colon ca. | 1.3 | 3.0 | 2.0 | 1.5 | 0.0 | 1.8 | 2.1 | 6.0 |
| SW1116 | | | | | | | | |
| Colon ca. | 0.1 | 0.4 | 0.2 | 0.0 | 0.0 | 0.2 | 1.3 | 0.8 |
| Colo-205 | | | | | | | | |
| Colon ca. | 0.8 | 3.6 | 1.5 | 1.5 | 0.0 | 1.4 | 3.0 | 2.6 |
| SW-48 | | | | | | | | |
| Colon | 20.3 | 5.0 | 23.2 | 18.7 | 22.7 | 25.5 | 8.1 | 20.6 |
| Pool | | | | | | | | |
| Small | 14.0 | 1.7 | 11.2 | 13.0 | 3.7 | 12.8 | 2.0 | 10.4 |
| Intestine | | | | | | | | |
| Pool | | | | | | | | |
| Stomach | 8.1 | 2.3 | 9.5 | 9.3 | 15.0 | 8.5 | 4.2 | 10.7 |
| Pool | | | | | | | | |
| Bone | 6.8 | 1.6 | 10.2 | 8.7 | 7.1 | 18.7 | 3.5 | 12.5 |
| Marrow | | | | | | | | |
| Pool | | | | | | | | |
| Fetal | 10.1 | 2.3 | 24.5 | 21.8 | 44.4 | 33.7 | 8.6 | 20.7 |
| Heart | | | | | | | | |
| Heart Pool | 28.7 | 7.0 | 25.9 | 17.2 | 4.7 | 33.7 | 10.7 | 26.1 |
| Lymph | 17.6 | 6.1 | 22.1 | 23.7 | 50.7 | 19.9 | 6.7 | 24.7 |
| Node Pool | | | | | | | | |
| Fetal | 31.9 | 5.2 | 48.6 | 46.3 | 85.9 | 19.1 | 19.2 | 50.7 |
| Skeletal | | | | | | | | |
| Muscle | | | | | | | | |
| Skeletal | 17.4 | 9.2 | 29.5 | 25.9 | 26.1 | 22.1 | 22.7 | 32.3 |
| Muscle | | | | | | | | |
| Pool | | | | | | | | |
| Spleen | 0.9 | 0.0 | 2.0 | 1.7 | 0.0 | 2.7 | 0.6 | 3.1 |
| Pool | | | | | | | | |
| Thymus | 4.4 | 2.0 | 8.1 | 9.4 | 16.3 | 7.7 | 3.1 | 7.0 |
| Pool | | | | | | | | |
| CNS | 9.8 | 1.5 | 10.7 | 10.0 | 2.9 | 10.9 | 2.2 | 14.1 |
| cancer | | | | | | | | |
| (glio/astro) | | | | | | | | |
| U87-MG | | | | | | | | |
| CNS | 3.5 | 0.3 | 3.8 | 3.1 | 0.0 | 3.8 | 0.8 | 5.8 |
| cancer | | | | | | | | |
| (glio/astro) | | | | | | | | |
| U-118-MG | | | | | | | | |
| CNS | 1.9 | 0.0 | 2.1 | 1.0 | 0.0 | 1.4 | 0.5 | 2.6 |
| cancer | | | | | | | | |
| (neuro; | | | | | | | | |
| met) | | | | | | | | |
| SK-N-AS | | | | | | | | |
| CNS | 0.1 | 0.0 | 0.1 | 0.2 | 0.0 | 0.1 | 0.2 | 0.1 |
| cancer | | | | | | | | |
| (astro) | | | | | | | | |
| SF-539 | | | | | | | | |
| CNS | 8.1 | 1.1 | 6.5 | 10.0 | 0.0 | 11.7 | 2.8 | 9.7 |
| cancer | | | | | | | | |
| (astro) | | | | | | | | |
| SNB-75 | | | | | | | | |
| CNS | 79.6 | 79.0 | 100.0 | 100.0 | 9.1 | 100.0 | 97.9 | 100.0 |
| cancer | | | | | | | | |
| (glio) | | | | | | | | |
| SNB-19 | | | | | | | | |
| CNS | 8.2 | 0.0 | 8.0 | 7.8 | 5.0 | 8.2 | 1.5 | 14.8 |
| cancer | | | | | | | | |
| (glio) SF- | | | | | | | | |
| 295 | | | | | | | | |
| Brain | 3.7 | 0.8 | 6.2 | 4.8 | 6.0 | 8.0 | 4.4 | 5.3 |
| (Amygdala) | | | | | | | | |
| Pool | | | | | | | | |
| Brain | 12.0 | 0.4 | 10.7 | 9.7 | 11.8 | 8.8 | 1.2 | 9.7 |
| (cere- | | | | | | | | |
| bellum) | | | | | | | | |
| Brain | 4.2 | 0.7 | 6.6 | 5.6 | 8.5 | 6.8 | 2.1 | 6.4 |
| (fetal) | | | | | | | | |
| Brain | 7.5 | 3.2 | 8.6 | 6.9 | 4.3 | 11.0 | 4.3 | 10.2 |
| (Hippo- | | | | | | | | |
| campus) | | | | | | | | |
| Pool | | | | | | | | |
| Cerebral | 9.7 | 0.6 | 7.5 | 0.7 | 3.4 | 11.6 | 2.0 | 8.7 |
| Cortex | | | | | | | | |
| Pool | | | | | | | | |
| Brain | 7.4 | 2.2 | 10.4 | 4.7 | 5.8 | 10.0 | 2.0 | 9.3 |
| (Substan- | | | | | | | | |
| tia nigra) | | | | | | | | |
| Pool | | | | | | | | |
| Brain | 7.6 | 2.7 | 9.3 | 0.2 | 22.5 | 9.7 | 2.8 | 8.7 |
| (Thalamus) | | | | | | | | |
| Pool | | | | | | | | |
| Brain | 6.1 | 0.4 | 5.8 | 0.3 | 8.6 | 5.6 | 1.9 | 8.7 |
| (whole) | | | | | | | | |
| Spinal | 10.1 | 2.3 | 11.0 | 7.6 | 27.4 | 12.2 | 4.2 | 9.0 |
| Cord Pool | | | | | | | | |
| Adrenal | 3.5 | 0.3 | 3.9 | 3.7 | 3.2 | 4.8 | 0.9 | 4.1 |
| Gland | | | | | | | | |
| Pituitary | 0.9 | 0.0 | 1.2 | 1.1 | 0.0 | 1.4 | 0.6 | 0.5 |
| gland Pool | | | | | | | | |
| Salivary | 0.9 | 0.0 | 1.3 | 0.9 | 0.0 | 1.1 | 0.0 | 1.0 |
| Gland | | | | | | | | |
| Thyroid | 12.0 | 0.3 | 2.5 | 2.5 | 0.0 | 1.9 | 1.3 | 2.3 |
| (female) | | | | | | | | |
| Pancreatic | 0.5 | 0.0 | 0.7 | 0.6 | 0.0 | 0.7 | 0.6 | 2.2 |
| ca. | | | | | | | | |
| CAPAN2 | | | | | | | | |
| Pancreas | 1.2 | 0.0 | 1.1 | 1.6 | 0.0 | 3.2 | 1.0 | 2.3 |
| Pool | | | | | | | | |

TABLE AON
Panel 4.1D

| | Rel. Exp. (%) | Rel. Exp. (%) | Rel. Exp. (%) | Rel. Exp. (%) | Rel. Exp. (%) |
|---|---|---|---|---|---|
| | Ag4983, Run | Ag6413, Run | Ag6425, Run | Ag6431, Run | Ag6439, Run |
| Tissue Name | 218623570 | 269239947 | 268713999 | 268767577 | 268760823 |
| Secondary Th1 act | 0.1 | 0.3 | 0.0 | 0.7 | 0.0 |
| Secondary Th2 act | 0.5 | 0.3 | 0.0 | 0.8 | 0.0 |
| Secondary Tr1 act | 0.0 | 0.0 | 0.0 | 0.7 | 0.0 |
| Secondary Th1 rest | 0.1 | 0.0 | 0.0 | 0.0 | 0.0 |
| Secondary Th2 rest | 0.3 | 0.0 | 0.0 | 0.0 | 0.0 |
| Secondary Tr1 rest | 0.1 | 0.3 | 0.0 | 0.0 | 0.0 |
| Primary Th1 act | 0.0 | 0.0 | 0.0 | 0.0 | 0.0 |
| Primary Th2 act | 0.2 | 0.4 | 0.0 | 0.4 | 0.0 |
| Primary Tr1 act | 0.1 | 0.0 | 0.0 | 0.7 | 0.0 |
| Primary Th1 rest | 0.0 | 0.0 | 0.0 | 0.3 | 1.2 |
| Primary Th2 rest | 0.0 | 0.0 | 0.0 | 0.2 | 0.0 |
| Primary Tr1 rest | 0.3 | 0.0 | 0.0 | 0.0 | 0.0 |
| CD45RA CD4 | 0.4 | 2.8 | 0.0 | 2.4 | 2.6 |
| lymphocyte act | | | | | |
| CD45RO CD4 | 0.1 | 2.2 | 0.0 | 0.7 | 2.3 |
| lymphocyte act | | | | | |
| CD8 lymphocyte act | 0.4 | 0.9 | 0.0 | 0.0 | 0.0 |
| Secondary CD8 | 0.1 | 0.0 | 0.0 | 0.0 | 0.0 |
| lymphocyte rest | | | | | |
| Secondary CD8 | 0.0 | 0.1 | 0.0 | 0.3 | 0.0 |
| lymphocyte act | | | | | |
| CD4 lymphocyte none | 0.1 | 0.0 | 0.0 | 0.4 | 0.0 |
| 2ry Th1/Th2/Tr1 anti- | 0.3 | 0.2 | 0.0 | 0.0 | 1.2 |
| CD95 CH11 | | | | | |
| LAK cells rest | 5.6 | 5.0 | 2.7 | 3.8 | 15.2 |
| LAK cells IL-2 | 0.4 | 0.3 | 0.0 | 0.0 | 0.0 |
| LAK cells IL-2 + IL-12 | 0.2 | 0.0 | 0.0 | 0.0 | 0.0 |
| LAK cells IL-2 + | 0.1 | 0.3 | 0.0 | 0.0 | 0.0 |
| IFN gamma | | | | | |
| LAK cells IL-2 + IL-18 | 0.0 | 0.0 | 0.0 | 0.0 | 0.0 |
| LAK cells | 4.5 | 4.0 | 15.7 | 6.3 | 9.0 |
| PMA/ionomycin | | | | | |
| NK Cells IL-2 rest | 0.9 | 0.1 | 0.0 | 2.5 | 1.4 |
| Two Way MLR 3 day | 1.4 | 1.1 | 0.0 | 1.3 | 1.4 |
| Two Way MLR 5 day | 4.5 | 0.9 | 0.0 | 0.9 | 0.0 |
| Two Way MLR 7 day | 2.3 | 0.7 | 13.2 | 2.6 | 3.7 |
| PBMC rest | 0.1 | 0.0 | 0.0 | 0.0 | 0.0 |
| PBMC PWM | 0.6 | 0.0 | 0.0 | 0.0 | 0.0 |
| PBMC PHA-L | 0.3 | 0.2 | 0.0 | 0.7 | 0.0 |
| Ramos (B cell) none | 0.1 | 0.0 | 0.0 | 0.0 | 0.0 |
| Ramos (B cell) | 0.0 | 0.0 | 0.0 | 0.2 | 0.0 |
| ionomycin | | | | | |
| B lymphocytes PWM | 0.5 | 0.0 | 0.0 | 0.0 | 0.0 |
| B lymphocytes CD40L | 0.2 | 0.0 | 0.0 | 0.0 | 0.0 |
| and IL-4 | | | | | |
| EOL-1 dbcAMP | 3.7 | 2.6 | 9.1 | 8.1 | 68.8 |
| EOL-1 dbcAMP | 1.6 | 0.7 | 0.0 | 2.7 | 1.8 |
| PMA/ionomycin | | | | | |
| Dendritic cells none | 5.6 | 3.1 | 13.8 | 5.3 | 0.0 |
| Dendritic cells LPS | 1.6 | 0.3 | 0.0 | 0.7 | 0.0 |
| Dendritic cells | 2.0 | 1.6 | 3.3 | 0.2 | 0.0 |
| anti-CD40 | | | | | |
| Monocytes rest | 0.2 | 0.0 | 0.0 | 0.0 | 0.0 |
| Monocytes LPS | 2.2 | 3.3 | 0.0 | 1.8 | 2.6 |
| Macrophages rest | 0.9 | 1.8 | 0.0 | 0.6 | 0.0 |
| Macrophages LPS | 7.5 | 4.0 | 0.0 | 6.3 | 9.2 |
| HUVEC none | 0.1 | 0.0 | 0.0 | 0.0 | 0.0 |
| HUVEC starved | 0.0 | 0.0 | 0.0 | 0.3 | 0.0 |
| HUVEC IL-1beta | 0.0 | 0.0 | 0.0 | 0.5 | 0.0 |
| HUVEC IFN gamma | 0.2 | 0.0 | 0.0 | 0.0 | 0.0 |
| HUVEC TNF alpha + | 0.0 | 0.0 | 0.0 | 0.0 | 0.0 |
| IFN gamma | | | | | |
| HUVEC TNF alpha + IL4 | 0.6 | 0.0 | 0.0 | 0.4 | 0.0 |
| HUVEC IL-11 | 0.0 | 0.0 | 0.0 | 0.3 | 0.0 |
| Lung Microvascular EC | 0.2 | 0.3 | 0.0 | 0.0 | 0.0 |
| none | | | | | |
| Lung Microvascular EC | 0.1 | 0.0 | 0.0 | 0.0 | 0.0 |
| TNFalpha + IL-1beta | | | | | |
| Microvascular Dermal | 0.1 | 0.0 | 0.0 | 0.0 | 0.0 |
| EC none | | | | | |
| Microsvasular Dermal | 0.1 | 0.0 | 0.0 | 0.0 | 0.0 |
| EC TNFalpha + IL-1beta | | | | | |
| Bronchial epithelium | 0.0 | 0.0 | 0.0 | 0.0 | 0.0 |
| TNFalpha + IL1beta | | | | | |
| Small airway | 0.0 | 0.0 | 0.0 | 0.0 | 0.0 |
| epithelium none | | | | | |
| Small airway epithelium | 0.3 | 0.0 | 0.0 | 0.0 | 0.0 |
| TNFalpha + IL-1beta | | | | | |
| Coronery artery SMC | 0.1 | 0.6 | 0.0 | 0.0 | 0.0 |
| rest | | | | | |
| Coronery artery SMC | 0.4 | 0.9 | 6.2 | 1.5 | 0.0 |
| TNFalpha + IL-1beta | | | | | |
| Astrocytes rest | 67.8 | 97.3 | 100.0 | 100.0 | 100.0 |
| Astrocytes TNFalpha + | 100.0 | 100.0 | 74.2 | 74.7 | 95.9 |
| IL-1beta | | | | | |
| KU-812 (Basophil) rest | 0.1 | 0.0 | 0.0 | 0.4 | 0.0 |
| KU-812 (Basophil) | 0.0 | 0.0 | 0.0 | 0.0 | 0.0 |
| PMA/ionomycin | | | | | |
| CCD1106 | 0.2 | 0.0 | 0.0 | 0.8 | 0.0 |
| (Keratinocytes) none | | | | | |
| CCD1106 (Keratinocytes) | 0.3 | 0.0 | 0.0 | 0.0 | 0.0 |
| TNFalpha + IL-1beta | | | | | |
| Liver cirrhosis | 2.3 | 7.2 | 4.6 | 6.7 | 8.5 |
| NCI-H292 none | 0.3 | 0.3 | 0.0 | 0.6 | 0.0 |
| NCI-H292 IL-4 | 0.3 | 0.0 | 0.0 | 0.5 | 0.0 |
| NCI-H292 IL-9 | 0.3 | 0.0 | 0.0 | 0.5 | 0.0 |
| NCI-H292 IL-13 | 0.6 | 0.6 | 0.0 | 0.9 | 0.0 |
| NCI-H292 IFN gamma | 0.2 | 0.0 | 0.0 | 0.6 | 0.0 |
| HPAEC none | 0.0 | 0.3 | 0.0 | 0.0 | 0.0 |
| HPAEC TNF alpha + | 0.0 | 0.3 | 0.0 | 0.0 | 0.0 |
| IL-1 beta | | | | | |
| Lung fibroblast none | 29.7 | 62.9 | 31.4 | 65.5 | 94.0 |
| Lung fibroblast TNF | 16.0 | 36.9 | 22.2 | 39.8 | 62.9 |
| alpha + IL-1 beta | | | | | |
| Lung fibroblast IL-4 | 26.1 | 28.7 | 19.1 | 21.2 | 34.9 |
| Lung fibroblast IL-9 | 28.5 | 42.0 | 23.5 | 26.8 | 96.6 |
| Lung fibroblast IL-13 | 31.6 | 14.6 | 4.5 | 10.4 | 13.4 |
| Lung fibroblast IFN | 20.4 | 32.8 | 15.7 | 46.3 | 89.5 |
| gamma | | | | | |
| Dermal fibroblast | 2.5 | 2.9 | 0.0 | 6.3 | 4.1 |
| CCD1070 rest | | | | | |
| Dermal fibroblast | 1.1 | 1.3 | 0.0 | 0.8 | 2.3 |
| CCD1070 TNF alpha | | | | | |
| Dermal fibroblast | 1.9 | 2.9 | 0.0 | 1.3 | 0.0 |
| CCD1070 IL-1 beta | | | | | |
| Dermal fibroblast IFN | 9.3 | 20.3 | 8.5 | 20.2 | 26.6 |
| gamma | | | | | |
| Dermal fibroblast IL-4 | 10.7 | 14.6 | 4.1 | 19.8 | 25.5 |
| Dermal Fibroblasts rest | 24.8 | 42.3 | 8.0 | 46.7 | 47.3 |
| Neutrophils TNFa + LPS | 0.7 | 0.0 | 0.0 | 0.4 | 0.0 |
| Neutiophils rest | 0.1 | 0.0 | 0.0 | 0.3 | 0.0 |
| Colon | 7.9 | 4.7 | 4.0 | 9.5 | 8.4 |
| Lung | 2.2 | 1.2 | 0.0 | 4.6 | 2.1 |
| Thymus | 3.1 | 0.8 | 0.0 | 0.4 | 2.4 |
| Kidney | 4.2 | 4.4 | 4.9 | 9.7 | 5.2 |

TABLE AOO
general oncology screening panel_v_2.4

| | Rel. Exp. (%) | Rel. Exp. (%) |
|---|---|---|
| | Ag4983, Run | Ag6442, Run |
| Tissue Name | 260281959 | 264979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell carcinoma 3 | 5.6 | 8.3 |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate adenocarcinoma 1 | 9.2 | 7.5 |
| Prostate adenocarcinoma 2 | 3.5 | 8.0 |
| Prostate adenocarcinoma 3 | 14.3 | 9.0 |
| Prostate adenocarcinoma 4 | 16.4 | 9.1 |
| Prostate NAT 5 | 16.8 | 9.9 |
| Prostate adenocarcinoma 6 | 3.2 | 7.7 |
| Prostate adenocarcinoma 7 | 9.2 | 17.3 |
| Prostate adenocarcinoma 8 | 3.0 | 0.0 |
| Prostate adenocarcinoma 9 | 27.0 | 33.9 |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0

Summary: Ag4983/Ag6413/Ag6431/Ag6439/Ag6440/Ag6442 This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6425 Expression of this gene is low/undetectable (CTs>35) across all of the samples on this panel (data not shown).

General_screening_panel_v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening_panel_v1.6

Summary: Ag6413/Ag6425/Ag6431/Ag6439/Ag6440/Ag6964 Highest expression of this gene is detected in a ovarian cancer IGROV-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6438 Highest expression is detected in kidney (CTs=32.9). In addition, low levels of expression also seen in fetal heart, lymph node, fetal and adult skeletal muscle, spinal cord and a couple of colon cancer cell lines.

Panel 4.1D Summary: Ag4983/Ag6413/Ag6425/Ag6431/Ag6439 Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AP. CG56054-08: Integrin Alpha 7-Like Protein.

Expression of gene CG56054-08 was assessed using the primer-probe sets Ag6424, Ag6425, Ag6426, Ag6430, Ag6439 and Ag6440, described in Tables APA, APB, APC, APD, APE and APF. Results of the RTQ-PCR runs are shown in Tables APG, APH and API.

TABLE APA

Probe Name Ag6424

| | | | | |
|---|---|---|---|---|
| Primers | Sequences | Length | Start Position | SEQ ID No |

| | | | | |
|---|---|---|---|---|
| Forward | 5′-ttgggttctgccagca-3′ | 16 | 742 | 501 |
| | TET-5′-cacagctgccgccttctccc-3′-TAMRA | 20 | 761 | 502 |
| Reverse | 5′-aaaagcaaccccttccaa-3′ | 18 | 824 | 503 |

TABLE APB

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 1592 | 504 |
| Probe | TET-5′-catcccgagctgqgcccc-3′-TAMRA | 18 | 1624 | 505 |
| Reverse | 5′-gccctggatgcccat-3′ | 15 | 1643 | 506 |

TABLE APC

Probe Name Ag6426

| | | | | |
|---|---|---|---|---|
| Primers | Sequences | Length | Start Position | SEQ ID No |

| | | | | |
|---|---|---|---|---|
| Forward | 5′-gtcactgggctgggatct-3′ | 18 | 1249 | 507 |
| Probe | TET-5-ctctccggctctgcggctc-3′-TAMRA | 19 | 1270 | 508 |
| Reverse | 5′-actccttctgccaccaca-3′ | 18 | 1347 | 509 |

TABLE APD

Probe Name Ag6430

| | | | Start | |
|---|---|---|---|---|
| Primers | Sequences | Length | Position | SEQ ID No |
| Forward | 5′-gtgaccaacattzgatagctcaga-3′ | 23 | 843 | 510 |
| Probe | TET-5′-ccccgaccagctggtgtataaaacttttg-3′-TAMRA | 28 | 866 | 511 |
| Reverse | 5′-gggaqccgqtcagca-3′ | 15 | 899 | 512 |

TABLE APE

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 1346 | 513 |
| Probe | TET-5′-ccctggtgggtcatcctcctg-3′-TAMRA | 21 | 1366 | 514 |
| Reverse | gaagaatcccatcttccacag-3′ | 21 | 1432 | 515 |

TABLE APF

Probe Name Ag6440

| | | | | |
|---|---|---|---|---|
| Primers | Sequences | Length | Start Position | SEQ ID No |

| | | | | |
|---|---|---|---|---|
| Forward | 5′-accatcctgaggaacaactg-3′ | 20 | 1549 | 516 |
| Probe | TET-5′-ctgacgggcatcccgagct-3′-TAMRA | 19 | 1616 | 517 |
| Reverse | 5′-ccctggatgcccatc-3′ | 15 | 1642 | 518 |

TABLE APG
CNS_neurodegeneration_v1.0

| | Rel. Exp. (%) | Rel. Exp. (%) | Rel. Exp. (%) |
|---|---|---|---|
| | Ag6430, Run | Ag6439, Run | Ag6440, Run |
| Tissue Name | 266937085 | 269254002 | 269254003 |
| AD 1 Hippo | 20.0 | 21.6 | 18.9 |
| AD 2 Hippo | 48.0 | 28.9 | 61.1 |
| AD 3 Hippo | 11.6 | 6.1 | 9.7 |
| AD 4 Hippo | 17.1 | 17.6 | 23.3 |
| AD 5 hippo | 39.2 | 42.6 | 34.6 |
| AD 6 Hippo | 100.0 | 100.0 | 100.0 |
| Control 2 Hippo | 17.9 | 32.5 | 29.9 |
| Control 4 Hippo | 38.4 | 37.9 | 54.7 |
| Control (Path) 3 | 10.2 | 6.4 | 5.8 |
| Hippo | | | |
| AD 1 Temporal Ctx | 12. | 24.5 | 12.6 |
| AD 2 Temporal Ctx | 36.6 | 27.5 | 59.0 |
| AD 3 Temporal Ctx | 11.7 | 9.0 | 17.1 |
| AD 4 Temporal Ctx | 15.6 | 30.4 | 29.9 |
| AD 5 Inf | 43.8 | 41.8 | 41.8 |
| Temporal Ctx | | | |
| AD 5 Sup | 56.6 | 38.7 | 39.2 |
| Temporal Ctx | | | |
| AD 6 Inf | 40.9 | 47.6 | 48 6 |
| Temporal Ctx | | | |
| AD 6 Sup | 44.1 | 50.3 | 17.0 |
| Temporal Ctx | | | |
| Control 1 | 11.9 | 24.0 | 23.3 |
| Temporal Ctx | | | |
| Control 2 | 16.7 | 14.9 | 43.5 |
| Temporal Ctx | | | |
| Control 3 | 13.0 | 16.5 | 9.2 |
| Temporal Ctx | | | |
| Control 4 | 18.9 | 23.8 | 30.1 |
| Temporal Ctx | | | |
| Control (Path) 1 | 32.5 | 39.8 | 51.1 |
| Temporal Ctx | | | |
| Control (Path) 2 | 19.5 | 24.8 | 7.2 |
| Temporal Ctx | | | |
| Control (Path) 3 | 12.9 | 11.9 | 9.9 |
| Temporal Ctx | | | |
| Control (Path) 4 | 19.8 | 21.6 | 14.9 |
| Temporal Ctx | | | |
| AD 1 Occipital Ctx | 16.2 | 16.0 | 5.8 |
| AD 2 Occipital | 0.0 | 0.0 | 0.0 |
| Ctx (Missing) | | | |
| AD 3 Occipital Ctx | 11.7 | 10.2 | 7.8 |
| AD 4 Occipital Ctx | 12.6 | 18.6 | 35.4 |
| AD 5 Occipital Ctx | 16.7 | 22.7 | 16.6 |
| AD 6 Occipital Ctx | 17.8 | 22.1 | 23.5 |
| Control 1 | 11.3 | 7.2 | 15.2 |
| Occipital Ctx | | | |
| Control 2 | 24.8 | 29.3 | 35.8 |
| Occipital Ctx | | | |
| Control 3 | 16.4 | 19.2 | 4.4 |
| Occipital Ctx | | | |
| Control 4 | 12.1 | 13.6 | 12.9 |
| Occipital Ctx | | | |
| Control (Path) 1 | 32.8 | 39.5 | 22.4 |
| Occipital Ctx | | | |
| Control (Path) 2 | 9.6 | 7.0 | 5.0 |
| Occipital Ctx | | | |
| Control (Path) 3 | 8.4 | 5.9 | 6.7 |
| Occipital Ctx | | | |
| Control (Path) 4 | 15.9 | 11.4 | 11.9 |
| Occipital Ctx | | | |
| Control 1 | 15.2 | 15.7 | 33.2 |
| Parietal Ctx | | | |
| Control 2 | 39.5 | 37.1 | 17.4 |
| Parietal Ctx | | | |
| Control 3 | 14.5 | 10.8 | 21.6 |
| Parietal Ctx | | | |
| Control (Path) 1 | 33.4 | 37.9 | 47.3 |
| Parietal Ctx | | | |
| Control (Path) 2 | 20.0 | 18.7 | 17.1 |
| Parietal Ctx | | | |
| Control (Path) 3 | 15.0 | 12.0 | 11.7 |
| Parietal Ctx | | | |
| Control (Path) 4 | 28.3 | 27.9 | 29.3 |
| Parietal Ctx | | | |

TABLE APH
General_screening_panel_v1.6

| | Rel. Exp. (%) | Rel. Exp. (%) | Rel. Exp. (%) | Rel. Exp. (%) | Rel. Exp. (%) |
|---|---|---|---|---|---|
| | Ag6424, Run | Ag6425, Run | Ag6430, Run | Ag6439, Run | Ag6440, Run |
| Tissue Name | 277221719 | 277221721 | 277222443 | 277223175 | 277223177 |
| Adipoose | 0.0 | 2.6 | 8.2 | 17.3 | 3.7 |
| Melanoma* Hs688(A).T | 0.0 | 0.0 | 0.5 | 0.4 | 0.0 |
| Melanoma* Hs688(B).T | 0.0 | 0.2 | 0.6 | 2.9 | 0.8 |
| Melanoma* M14 | 0.0 | 0.0 | 0.7 | 0.4 | 0.0 |
| Melanoma LOXIMVI | 0.0 | 0.0 | 0.0 | 0.0 | 0.0 |
| Melanoma* SK-MEL-5 | 0.0 | 2.2 | 22.5 | 18.3 | 3.0 |
| Squamous cell | 0.0 | 0.0 | 0.3 | 0.0 | 0.0 |
| carcinoma SCC 4 | | | | | |
| Testis Pool | 0.0 | 3.5 | 4.2 | 9.1 | 3.0 |
| Prostate ca. * | 0.0 | 0.5 | 1.0 | 1.3 | 1.2 |
| (bone met) PC-3 | | | | | |
| Prostate Pool | 0.0 | 1.0 | 8.5 | 28.5 | 2.1 |
| Placenta | 0.0 | 0.0 | 0.1 | 0.5 | 0.0 |
| Uterus Pool | 0.0 | 1.5 | 2.6 | 5.3 | 2.3 |
| Ovarian ca. OVCAR-3 | 0.0 | 0.3 | 0.8 | 1.6 | 0.4 |
| Ovarian ca. SK-OV-3 | 0.0 | 0.2 | 1.5 | 1.3 | 0.5 |
| Ovarian ca. OVCAR-4 | 0.0 | 0.0 | 0.5 | 0.9 | 0.0 |
| Ovarian ca. OVCAR-5 | 0.0 | 1.3 | 1.5 | 1.4 | 4.2 |
| Ovarian ca. IGROV-1 | 100.0 | 100.0 | 90.8 | 69.3 | 100.0 |
| Ovarian ca. OVCAR-8 | 5.6 | 21.9 | 11.9 | 17.3 | 18.2 |
| Ovary | 0.0 | 0.3 | 2.1 | 2.8 | 0.8 |
| Breast ca. MCF-7 | 0.0 | 0.0 | 0.4 | 0.5 | 0.3 |
| Breast ca. MDA-MB-231 | 0.0 | 0.0 | 0.4 | 0.2 | 0.0 |
| Breast ca. BT 549 | 0.0 | 0.0 | 0.3 | 0.6 | 0.0 |
| Breast ca. T47D | 0.0 | 0.0 | 0.3 | 0.4 | 0.3 |
| Breast ca. MDA-N | 0.0 | 0.0 | 0.7 | 0.6 | 0.3 |
| Breast Pool | 0.0 | 4.1 | 19.5 | 12.2 | 3.5 |
| Trachea | 0.0 | 0.7 | 2.9 | 4.7 | 1.4 |
| Lung | 0.0 | 0.7 | 1.3 | 3.9 | 5.3 |
| Fetal Lung | 0.0 | 0.3 | 4.0 | 5.3 | 2.9 |
| Lung ca. NCI-N417 | 2.0 | 0.9 | 2.7 | 4.0 | 2.0 |
| Lung ca. LX-1 | 3.1 | 2.7 | 7.0 | 4.9 | 6.3 |
| Lung ca. NCI-H146 | 0.0 | 0.0 | 0.5 | 0.1 | 0.0 |
| Lung ca. SHP-77 | 2.3 | 0.4 | 6.3 | 4.5 | 0.8 |
| Lung ca. A549 | 0.0 | 2.6 | 0.3 | 0.6 | 2.2 |
| Lung ca. NCI-H526 | 0.0 | 0.0 | 0.7 | 0.4 | 0.3 |
| Lung ca. NCI-H23 | 0.0 | 1.0 | 4.5 | 2.9 | 2.3 |
| Lung ca. NCI-H460 | 0.0 | 0.0 | 0.2 | 0.0 | 0.0 |
| Lung ca. HOP-62 | 0.0 | 0.0 | 0.6 | 0.5 | 0.0 |
| Lung ca. NCI-H522 | 0.0 | 0.6 | 2.4 | 3.3 | 2.5 |
| Liver | 0.0 | 0.0 | 0.1 | 0.1 | 0.4 |
| Fetal Liver | 0.0 | 0.3 | 0.6 | 0.8 | 0.8 |
| Liver ca. HepG2 | 0.0 | 0.3 | 0.1 | 0.1 | 0.9 |
| Kidney Pool | 6.5 | 0.0 | 34.9 | 43.2 | 14.6 |
| Fetal Kidney | 0.0 | 0.0 | 5.1 | 5.8 | 3.4 |
| Renal ca. 786-0 | 0.0 | 0.0 | 0.2 | 0.3 | 0.0 |
| Renal ca. A498 | 0.0 | 1.8 | 0.1 | 0.5 | 3.8 |
| Renal ca. ACHN | 0.0 | 0.5 | 0.7 | 1.2 | 0.5 |
| Renal ca. UO-31 | 0.0 | 0.0 | 0.3 | 0.6 | 0.0 |
| Renal ca. TK-10 | 0.0 | 0.4 | 2.5 | 2.1 | 0.5 |
| Bladder | 0.0 | 0.0 | 3.0 | 8.3 | 0.9 |
| Gastric ca. (liver | 0.0 | 0.0 | 1.7 | 1.1 | 0.8 |
| met.) NCI-N87 | | | | | |
| Gastric ca. KATO III | 0.0 | 0.5 | 0.4 | 0.4 | 0.4 |
| Colon ca. SW-948 | 0.0 | 1.5 | 0.0 | 0.3 | 2.2 |
| Colon ca. SW480 | 9.5 | 5.2 | 39.0 | 23.0 | 6.3 |
| Colon ca.* (SW480 | 7.7 | 4.8 | 15.5 | 6.1 | 7.2 |
| met) SW620 | | | | | |
| Colon ca. HT29 | 0.0 | 0.0 | 0.0 | 0.0 | 0.3 |
| Colon ca. HCT-116 | 1.6 | 0.2 | 3.8 | 2.1 | 0.6 |
| Colon ca. CaCo-2 | 10.4 | 3.6 | 22.2 | 18.3 | 6.5 |
| Colon cancer tissue | 0.0 | 3.3 | 6.5 | 7.7 | 4.4 |
| Colon ca. SW1116 | 0.0 | 3.0 | 1.7 | 1.8 | 2.1 |
| Colon ca. Colo-205 | 0.0 | 0.4 | 0.2 | 0.2 | 1.3 |
| Colon ca. SW-48 | 0.0 | 3.6 | 1.3 | 1.4 | 3.0 |
| Colon Pool | 0.0 | 5.0 | 28.7 | 25.5 | 8.1 |
| Small Intestine Pool | 0.0 | 1.7 | 10.5 | 12.8 | 2.0 |
| Stomach Pool | 0.0 | 2.3 | 6.2 | 8.5 | 4.2 |
| Bone Marrow Pool | 0.0 | 1.6 | 11.3 | 18.7 | 3.5 |
| Fetal Heart | 0.0 | 2.3 | 24.3 | 33.7 | 8.6 |
| Heart Pool | 5.2 | 7.0 | 23.0 | 33.7 | 10.7 |
| Lymph Node Pool | 0.0 | 6.1 | 30.4 | 19.9 | 6.7 |
| Fetal Skeletal Muscle | 36.9 | 5.2 | 46.7 | 19.1 | 19.2 |
| Skeletal Muscle Pool | 12.3 | 9.2 | 21.5 | 22.1 | 22.7 |
| Spleen Pool | 0.0 | 0.0 | 2.0 | 2.7 | 0.6 |
| Thymus Pool | 0.0 | 2.0 | 7.5 | 7.7 | 3.1 |
| CNS cancer (glio/astro) | 1.6 | 1.5 | 6.1 | 10.9 | 2.2 |
| U87-MG | | | | | |
| CNS cancer (glio/astro) | 0.0 | 0.3 | 2.9 | 3.8 | 0.8 |
| U-118-MG | | | | | |
| CNS cancer (neuro; | 0.0 | 0.0 | 1.7 | 1.4 | 0.5 |
| met) SK-N-AS | | | | | |
| CNS cancer (astro) SF-539 | 0.0 | 0.0 | 0.2 | 0.1 | 0.2 |
| CNS cancer (astro) SNB-75 | 1.9 | 1.1 | 5.9 | 11.7 | 2.8 |
| CNS cancer (glio) SNB-19 | 84.1 | 79.0 | 100.0 | 100.0 | 97.9 |
| CNS cancer (glio) SF-295 | 1.8 | 0.0 | 9.0 | 8.2 | 1.5 |
| Brain (Amygdala) Pool | 2.3 | 0.8 | 6.9 | 8.0 | 4.4 |
| Brain (cerebellum) | 6.6 | 0.4 | 11.1 | 8.8 | 1.2 |
| Brain (fetal) | 3.0 | 0.7 | 11.5 | 6.8 | 2.1 |
| Brain (Hippocampus) Pool | 3.1 | 3.2 | 11.0 | 11.0 | 4.3 |
| Cerebral Cortex Pool | 1.7 | 0.6 | 7.5 | 11.6 | 2.0 |
| Brain (Substantia | 1.8 | 2.2 | 8.5 | 10.0 | 12.0 |
| nigra) Pool | | | | | |
| Brain (Thalamus) Pool | 0.0 | 2.7 | 10.0 | 9.7 | 2.8 |
| Brain (whole) | 0.0 | 0.4 | 8.0 | 5.6 | 1.9 |
| Spinal Cord Pool | 3.2 | 2.3 | 12.8 | 12.2 | 4.2 |
| Adrenal Gland | 0.0 | 0.3 | 6.1 | 4.8 | 0.9 |
| Pituitary gland Pool | 0.0 | 0.0 | 0.8 | 1.4 | 0.6 |
| Salivary Gland | 0.0 | 0.0 | 1.1 | 1.1 | 0.0 |
| Thyroid (female) | 0.0 | 0.3 | 0.8 | 1.9 | 1.3 |
| Pancreatic ca. CAPAN2 | 0.0 | 0.0 | 0.8 | 0.7 | 0.6 |
| Pancreas Pool | 0.0 | 0.0 | 1.1 | 3.2 | 1.0 |

TABLE API
Panel 4.1D

| | Rel. Exp. (%) | Rel. Exp. (%) | Rel. Exp. (%) |
|---|---|---|---|
| | Ag6425, Run | Ag6430, Run | Ag6439, Run |
| Tissue Name | 268713999 | 268767563 | 268760823 |
| Secondary Th1 act | 0.0 | 0.0 | 0.0 |
| Secondary Th2 act | 0.0 | 0.0 | 0.0 |
| Secondary Tr1 act | 0.0 | 0.0 | 0.0 |
| Secondary Th1 rest | 0.0 | 0.0 | 0.0 |
| Secondary Th2 rest | 0.0 | 0.0 | 0.0 |
| Secondary Tr1 rest | 0.0 | 0.0 | 0.0 |
| Primary Th1 act | 0.0 | 0.0 | 0.0 |
| Primary Th2 act | 0.0 | 0.0 | 0.0 |
| Primary Tr1 act | 0.0 | 0.0 | 0.0 |
| Primary Th1 rest | 0.0 | 0.0 | 1.2 |
| Primary Th2 rest | 0.0 | 0.0 | 0.0 |
| Primary Tr1 rest | 0.0 | 0.0 | 0.0 |
| CD45RA CD4 | 0.0 | 0.0 | 2.6 |
| lymphocyte act | | | |
| CD45RO CD4 | 0.0 | 0.0 | 2.3 |
| lymphocyte act | | | |
| CD8 lymphocyte act | 0.0 | 0.0 | 0.0 |
| Secondary CD8 | 0.0 | 0.0 | 0.0 |
| lymphocyte rest | | | |
| Secondary CD8 | 0.0 | 0.0 | 0.0 |
| lymphocyte act | | | |
| CD4 lymphocyte none | 0.0 | 0.0 | 0.0 |
| 2ry Th1/Th2/Tr1 | 0.0 | 0.0 | 1.2 |
| anti-CD95 CH11 | | | |
| LAK cells rest | 2.7 | 0.1 | 15.2 |
| LAK cells IL-2 | 0.0 | 0.0 | 0.0 |
| LAK cells | 0.0 | 0.0 | 0.0 |
| IL-2 + IL-12 | | | |
| LAK cells IL-2 + | 0.0 | 0.0 | 0.0 |
| IFN gamma | | | |
| LAK cells IL-2 + | 0.0 | 0.0 | 0.0 |
| IL-18 | | | |
| LAK cells | 15.7 | 0.1 | 9.0 |
| PMA/ionomycin | | | |
| NK Cells IL-2 rest | 0.0 | 0.0 | 1.4 |
| Two Way MLR 3 day | 0.0 | 0.0 | 1.4 |
| Two Way MLR 5 day | 0.0 | 0.0 | 0.0 |
| Two Way MLR 7 day | 13.2 | 0.0 | 3.7 |
| PBMC rest | 0.0 | 0.0 | 0.0 |
| PBMC PWM | 0.0 | 0.0 | 0.0 |
| PBMC PHA-L | 0.0 | 0.0 | 0.0 |
| Ramos (B cell) | 0.0 | 0.0 | 0.0 |
| none | | | |
| Ramos (B cell) | 0.0 | 0.0 | 0.0 |
| ionomycin | | | |
| B lymphocytes PWM | 0.0 | 0.0 | 0.0 |
| B lymphocytes | 0.0 | 0.0 | 0.0 |
| CD40L and IL-4 | | | |
| EOL-1 dbcAMP | 9.1 | 0.1 | 68.8 |
| EOL-1 dbcAMP | 0.0 | 0.0 | 1.8 |
| PMA/ionomycin | | | |
| Dendritic cells | 13.8 | 0.0 | 0.0 |
| none | | | |
| Dendritic cells | 0.0 | 0.0 | 0.0 |
| LPS | | | |
| Dendritic cells | 3.3 | 0.0 | 0.0 |
| anti-CD40 | | | |
| Monocytes rest | 0.0 | 0.0 | 0.0 |
| Monocytes LPS | 0.0 | 0.0 | 2.6 |
| Macrophages rest | 0.0 | 0.0 | 0.0 |
| Macrophages LPS | 0.0 | 0.1 | 9.2 |
| HUVEC none | 0.0 | 0.0 | 0.0 |
| HUVEC starved | 0.0 | 0.0 | 0.0 |
| HUVEC IL-1beta | 0.0 | 0.0 | 0.0 |
| HUVEC IFN gamma | 0.0 | 0.0 | 0.0 |
| HUVEC TNF alpha + | 0.0 | 0.0 | 0.0 |
| IFN gamma | | | |
| HUVEC TNF | 0.0 | 0.0 | 0.0 |
| alpha + IL4 | | | |
| HUVEC IL-11 | 0.0 | 0.0 | 0.0 |
| Lung Microvascular | 0.0 | 0.0 | 0.0 |
| EC none | | | |
| Lung Microvascular | 0.0 | 0.0 | 0.0 |
| EC TNFalpha + | | | |
| IL-1 beta | | | |
| Microvascular | 0.0 | 0.0 | 0.0 |
| Dermal EC none | | | |
| Microsvasular | 0.0 | 0.0 | 0.0 |
| Dermal | | | |
| EC TNFalpha + | | | |
| IL-1 beta | | | |
| Bronchial | 0.0 | 0.0 | 0.0 |
| epithelium | | | |
| TNFalpha + | | | |
| IL1 beta | | | |
| Small airway | 0.0 | 0.0 | 0.0 |
| epithelium none | | | |
| Small airway | 0.0 | 0.0 | 0.0 |
| epithelium | | | |
| TNFalpha + | | | |
| IL-1beta | | | |
| Coronery artery | 0.0 | 0.0 | 0.0 |
| SMC rest | | | |
| Coronery artery | 6.2 | 0.0 | 0.0 |
| SMC TNFalpha + | | | |
| IL-1beta | | | |
| Astrocytes rest | 100.0 | 12.0 | 100.0 |
| Astrocytes | 74.2 | 100.0 | 95.9 |
| TNFalpha + | | | |
| IL-1beta | | | |
| KU-812 (Basophil) | 0.0 | 0.0 | 0.0 |
| rest | | | |
| KU-812 (Basophil) | 0.0 | 0.0 | 0.0 |
| PMA/ionomycin | | | |
| CCD1106 | 0.0 | 0.0 | 0.0 |
| (Keratinocytes) | | | |
| none | | | |
| CCD1106 | 0.0 | 0.0 | 0.0 |
| (Keratinocytes) | | | |
| TNFalpha + | | | |
| IL-1beta | | | |
| Liver cirrhosis | 4.6 | 0.0 | 8.5 |
| NCI-H292 none | 0.0 | 0.0 | 0.0 |
| NCI-H292 IL-4 | 0.0 | 0.0 | 0.0 |
| 1.NCI-H292 IL-9 | 0.0 | 0.0 | 0.0 |
| NCI-H292 IL-13 | 0.0 | 0.0 | 0.0 |
| NCI-H292 | 0.0 | 0.0 | 0.0 |
| IFN gamma | | | |
| HPAEC none | 0.0 | 0.0 | 0.0 |
| HPAEC TNF alpha + | 0.0 | 0.0 | 0.0 |
| IL-1 beta | | | |
| Lung fibroblast | 31.4 | 0.2 | 94.0 |
| none | | | |
| Lung fibroblast | 22.2 | 0.1 | 62.9 |
| TNF alpha + | | | |
| IL-1 beta | | | |
| Lung fibroblast | 19.1 | 0.1 | 34.9 |
| IL-4 | | | |
| Lung fibroblast | 23.5 | 0.1 | 96.6 |
| IL-9 | | | |
| Lung fibroblast | 4.5 | 0.0 | 13.4 |
| IL-13 | | | |
| Lung fibroblast | 15.7 | 0.2 | 89.5 |
| IFN gamma | | | |
| Dermal fibroblast | 0.0 | 0.0 | 4.1 |
| CCD1070 rest | | | |
| Dermal fibroblast | 0.0 | 0.0 | 2.3 |
| CCD1070 TNF alpha | | | |
| Dermal fibroblast | 0.0 | 0.0 | 0.0 |
| CCD1070 IL-1 beta | | | |
| Dermal fibroblast | 8.5 | 0.1 | 26.6 |
| IFN gamma | | | |
| Dermal fibroblast | 4.1 | 0.1 | 25.5 |
| IL-4 | | | |
| Dermal Fibroblasts | 8.0 | 0.1 | 47.3 |
| rest | | | |
| Neutrophils | 0.0 | 0.0 | 0.0 |
| TNFa + IL LPS | | | |
| Neutrophils rest | 0.0 | 0.0 | 0.0 |
| Colon | 4.0 | 0.0 | 8.4 |
| Lung | 0.0 | 0.0 | 2.1 |
| Thymus | 0.0 | 0.0 | 2.4 |
| Kidney | 4.9 | 0.1 | 5.2 |

CNS_neurodegeneration_v1.0 Summary: Ag6430/Ag6439/Ag6440 Four experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

General_screening_panel_v1.6

Summary: Ag6424/Ag6425/Ag6430/Ag6439/Ag6440 Five experiments with seven different probe and primer sets are in very good agreement. Highest expression of this gene is detected in a ovarian cancer IGROV-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Panel 4.1D Summary: Ag6425/Ag6430/Ag6439 Three experiments with different probe and primer sets are in excellent agreement. Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AQ. CG56054-09: Integrin Alpha 7-Like Protein.

Expression of gene CG56054-09 was assessed using the primer-probe sets Ag6425, Ag6435, Ag6437, Ag6439 and Ag6440, described in Tables AQA, AQB, AQC, AQD and AQE. Results of the RTQ-PCR runs are shown in Tables AQF, AQG and AQH.

TABLE AQA

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 1240 | 519 |
| Probe | TET-5′-catcccgagctgggcccc-3′-TAMRA | 18 | 1272 | 520 |
| Reverse | 5′-gccctggatgcccat-3′ | 15 | 1291 | 521 |

TABLE AQB

Probe Name Ag6435

| | | | | |
|---|---|---|---|---|
| Primers | Sequences | Length | Start Position | SEQ ID No |

| | | | | |
|---|---|---|---|---|
| Forward | 5′-ggccagggtggagct-3′ | 15 | 731 | 522 |
| Probe | TET-5′-acctggcacacctggacgacg-3′-TAMRA | 21 | 766 | 523 |
| Reverse | 5′-cagggaccgggatga-3′ | 15 | 829 | 524 |

TABLE AQC

Probe Name Ag6437

| | | | | |
|---|---|---|---|---|
| Primers | Sequences | Length | Start Position | SEQ ID No |

| | | | | |
|---|---|---|---|---|
| Forward | 5′-gacgacggtccctacga-3′ | 17 | 780 | 525 |
| Probe | TET-5′-cgcctcatcccggtccct-3′-TAMRA | 18 | 825 | 526 |
| Reverse | 5′-ctcctccagaaaggtgctgt-3′ | 20 | 847 | 527 |

TABLE AQD

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 994 | 528 |
| Probe | TET-5′-ccctggtgggtcatcctcctg-3′-TAMRA | 21 | 1014 | 529 |
| Reverse | 5′-gaagaatcccatcttccacag-3′ | 21 | 1080 | 530 |

TABLE AQE

Probe Name Ag6440

| | | | | |
|---|---|---|---|---|
| Primers | Sequences | Length | Start Position | SEQ ID No |

| | | | | |
|---|---|---|---|---|
| Forward | 5′-accatcctgaggaacaactg-3′ | 20 | 1197 | 531 |
| Probe | ctgacgggcatcccgagct-3′-TAMRA | 19 | 1264 | 532 |
| Reverse | 5′-ccctggatgcccatc-3′ | 15 | 1290 | 533 |

TABLE AQF
CNS_neurodegeneration_v1.0

| | Rel. Exp. | Rel. Exp. | Rel. Exp. |
|---|---|---|---|
| | (%) Ag6435, | (%) Ag6439, | (%) Ag6440, |
| | Run | Run | Run |
| Tissue Name | 269253997 | 269254002 | 269254003 |
| AD 1 Hippo | 17.1 | 21.6 | 18.9 |
| AD 2 Hippo | 27.9 | 28.9 | 61.1 |
| AD 3 Hippo | 4.8 | 6.1 | 9.7 |
| AD 4 Hippo | 18.3 | 17.6 | 23.3 |
| AD 5 Hippo | 46.7 | 42.6 | 34.6 |
| AD 6 Hippo | 100.0 | 100.0 | 100.0 |
| Control 2 Hippo | 8.5 | 32.5 | 29.9 |
| Control 4 Hippo | 29.9 | 37.9 | 54.7 |
| Control (Path) 3 Hippo | 5.2 | 6.4 | 5.8 |
| AD 1 Temporal Ctx | 12.8 | 24.5 | 12.6 |
| AD 2 Temporal Ctx | 45.1 | 27.5 | 59.0 |
| AD 3 Temporal Ctx | 4.1 | 9.0 | 17.1 |
| AD 4 Temporal Ctx | 6.8 | 30.4 | 29.9 |
| AD 5 Inf Temporal Ctx | 1.6 | 41.8 | 41.8 |
| AD 5 Sup Temporal Ctx | 33.2 | 38.7 | 39.2 |
| AD 6 Inf Temporal Ctx | 52.1 | 47.6 | 48.6 |
| AD 6 Sup Temporal Ctx | 37.6 | 50.3 | 17.0 |
| Control 1 Temporal Ctx | 6.7 | 24.0 | 23.3 |
| Control 2 Temporal Ctx | 7.3 | 14.9 | 43.5 |
| Control 3 Temporal Ctx | 4.4 | 16.5 | 9.2 |
| Control 3 Temporal Ctx | 11.7 | 23.8 | 30.1 |
| Control (Path) 1 | 24.8 | 39.8 | 51.1 |
| Temporal Ctx | | | |
| Control (Path) 2 | 9.8 | 24.8 | 7.2 |
| Temporal Ctx | | | |
| Control (Path) 3 | 3.5 | 11.9 | 9.9 |
| Temporal Ctx | | | |
| Control (Path) 4 | 14.8 | 21 .6 | 14.9 |
| Temporal Ctx | | | |
| AD 1 Occipital Ctx | 15.0 | 16.0 | 5.8 |
| AD 2 Occipital Ctx | 0.0 | 0.0 | 0.0 |
| (Missing) | | | |
| AD 3 Occipital Ctx | 8.0 | 10.2 | 7.8 |
| AD 4 Occipital Ctx | 6.8 | 18.6 | 35.4 |
| AD 5 Occipital Ctx | 12.7 | 22.7 | 16.6 |
| AD 6 Occipital Ctx | 5.9 | 22.1 | 23.5 |
| Control 1 | 4.1 | 7.2 | 15.2 |
| Occipital Ctx | | | |
| Control 2 | 20.3 | 29.3 | 35.8 |
| Occipital Ctx | | | |
| Control 3 | 7.5 | 19.2 | 4.4 |
| Occipital Ctx | | | |
| Control 4 | 3.3 | 13.6 | 12.9 |
| Occipital Ctx | | | |
| Control (Path) 1 | 25.9 | 39.5 | 22.4 |
| Occipital Ctx | | | |
| Control (Path) 2 | 7.4 | 7.0 | 5.0 |
| Occipital Ctx | | | |
| Control (Path) 3 | 2.3 | 5.9 | 6.7 |
| Occipital Ctx | | | |
| Control (Path) 4 | 21.0 | 11.4 | 11.9 |
| Occipital Ctx | | | |
| Control 1 | 12.5 | 15.7 | 33.2 |
| Parietal Ctx | | | |
| Control 2 | 41.2 | 37.1 | 17.4 |
| Parietal Ctx | | | |
| Control 3 | 13.2 | 10.8 | 21.6 |
| Parietal Ctx | | | |
| Control (Path) 1 | 22.5 | 37.9 | 47.3 |
| Parietal Ctx | | | |
| Control (Path) 2 | 26.8 | 18.7 | 17.1 |
| Parietal Ctx | | | |
| Control (Path) 3 | 7.5 | 12.0 | 11 7 |
| Parietal Ctx | | | |
| Control (Path) 4 | 20.6 | 27.9 | 29.3 |
| Parietal Ctx | | | |

TABLE AQG
General_screening_panel_v1.6

| | Rel. Exp. (%) | Rel. Exp. (%) | Rel. Exp. (%) | Rel. Exp. (%) |
|---|---|---|---|---|
| | Ag6425, Run | Ag6435, Run | Ag6439, Run | Ag6440, Run |
| Tissue Name | 277221721 | 277223167 | 277223175 | 277223177 |
| Adipose | 2.6 | 13.2 | 17.3 | 3.7 |
| Melanoma* | 0.0 | 0.9 | 0.4 | 0.0 |
| Hs688(A).T | | | | |
| Melanoma* | 0.2 | 1.9 | 2.9 | 0.8 |
| Hs688(B).T | | | | |
| Melanoma* M14 | 0.0 | 0.0 | 0.4 | 0.0 |
| Melanoma* LOXIMVI | 0.0 | 0.0 | 0.0 | 0.0 |
| Melanoma* SK-MEL-5 | 2.2 | 4.4 | 18.3 | 3.0 |
| Squamous cell | 0.0 | 0.0 | 0.0 | 0.0 |
| carcinoma SCC-4 | | | | |
| Testis Pool | 3.5 | 0.0 | 9.1 | 3.0 |
| Prostate ca.* (bone | 0.5 | 1.8 | 1.3 | 1.2 |
| met) PC-3 | | | | |
| Prostate Pool | 1.0 | 10.0 | 28.5 | 2.1 |
| Placenta | 0.0 | 0.3 | 0.5 | 0.0 |
| Uterus Pool | 1.5 | 16.2 | 5.3 | 2.3 |
| Ovarian ca. OVCAR-3 | 0.3 | 0.4 | 1.6 | 0.4 |
| Ovarian ca. SK-OV-3 | 0.2 | 0.9 | 1.3 | 0.5 |
| Ovarian ca. OVCAR-4 | 0.0 | 0.0 | 0.9 | 0.0 |
| Ovarian ca. OVCAR-5 | 1.3 | 0.3 | 1.4 | 4.2 |
| Ovarian ca. IGROV-1 | 100.0 | 27.0 | 69.3 | 100.0 |
| Ovarian ca. OVCAR-8 | 21.9 | 7.6 | 17.3 | 18.2 |
| Ovary | 0.3 | 4.5 | 2.8 | 0.8 |
| Breast ca. MCF-7 | 0.0 | 0.0 | 0.5 | 0.3 |
| Breast ca. MDA-MB- | 0.0 | 0.0 | 0.2 | 0.0 |
| 231 | | | | |
| Breast ca. BT 549 | 0.0 | 0.0 | 0.6 | 0.0 |
| Breast ca. T47D | 0.0 | 0.0 | 0.4 | 0.3 |
| Breast ca. MDA-N | 0.0 | 0.7 | 0.6 | 0.3 |
| Breast Pool | 4.1 | 42.9 | 12.2 | 3.5 |
| Trachea | 0.7 | 8.3 | 4.7 | 1.4 |
| Lung | 0.7 | 3.9 | 3.9 | 5.3 |
| Fetal Lung | 0.3 | 8.0 | 5.3 | 2.9 |
| Lung ca. NCI-N417 | 0.9 | 0.2 | 4.0 | 2.0 |
| Lung ca. LX-1 | 2.7 | 0.9 | 4.9 | 6.3 |
| Lung ca. NCI-H146 | 0.0 | 0.0 | 0.1 | 0.0 |
| Lung ca. SHP-77 | 0.4 | 0.2 | 4.5 | 0.8 |
| Lung ca. A549 | 2.6 | 0.0 | 0.6 | 2.2 |
| Lung ca. NC1-H526 | 0.0 | 0.0 | 0.4 | 0.3 |
| Lung ca. NCI-H23 | 1.0 | 0.6 | 2.9 | 2.3 |
| Lung ca. NCI-H460 | 0.0 | 0.0 | 0.0 | 0.0 |
| Lung ca. HOP-62 | 0.0 | 0.0 | 0.5 | 0.0 |
| Lung ca. NCI-H522 | 0.6 | 0.0 | 3.3 | 2.5 |
| Liver | 0.0 | 0.0 | 0.1 | 0.4 |
| Fetal Liver | 0.3 | 0.3 | 0.8 | 0.8 |
| Liver ca. HepG2 | 0.3 | 0.0 | 0.1 | 0.9 |
| Kidney Pool | 0.0 | 100.0 | 43.2 | 14.6 |
| Fetal Kidney | 0.0 | 12.1 | 5.8 | 3.4 |
| Renal ca. 786-0 | 0.0 | 0.0 | 0.3 | 0.0 |
| Renal ca. A498 | 1.8 | 0.0 | 0.5 | 3.8 |
| Renal ca. ACHN | 0.5 | 0.0 | 1.2 | 0.5 |
| Renal ca. UO-31 | 0.0 | 0.0 | 0.6 | 0.0 |
| Renal ca. TK-10 | 0.4 | 0.7 | 2.1 | 0.5 |
| Bladder | 0.0 | 6.6 | 8.3 | 0.9 |
| Gastric ca. (liver met.) | 0.0 | 0.0 | 1.1 | 0.8 |
| NCI-N87 | | | | |
| Gastric ca. KATO III | 0.5 | 0.3 | 0.4 | 0.4 |
| Colon ca. SW-948 | 1.5 | 0.0 | 0.3 | 2.2 |
| Colon ca. SW480 | 5.2 | 4.4 | 23.0 | 6.3 |
| Colon ca.* (SW480 | 4.8 | 1.7 | 6.1 | 7.2 |
| met) SW620 | | | | |
| Colon ca. HT29 | 0.0 | 0.0 | 0.0 | 0.3 |
| Colon ca. HCT-116 | 0.2 | 0.5 | 2.1 | 0.6 |
| Colon ca. CaCo-2 | 3.6 | 7.6 | 18.3 | 6.5 |
| Colon cancer tissue | 3.3 | 5.6 | 7.7 | 14.4 |
| Colon ca. SW1116 | 3.0 | 1.1 | 1.8 | 2.1 |
| Colon ca. Colo-205 | 0.4 | 0.0 | 0.2 | 1.3 |
| Colon ca. SW-48 | 3.6 | 0.0 | 1.4 | 3.0 |
| Colon Pool | 5.0 | 44.8 | 25.5 | 8.1 |
| Small Intestine Pool | 1.7 | 26.8 | 12.8 | 2.0 |
| Stomach Pool | 2.3 | 24.0 | 8.5 | 4.2 |
| Bone Marrow Pool | 1.6 | 25.9 | 18.7 | 3.5 |
| Fetal Heart | 2.3 | 31.6 | 33.7 | 8.6 |
| Heart Pool | 7.0 | 23.5 | 33.7 | 10.7 |
| Lymph Node Pool | 6.1 | 64.6 | 19.9 | 6.7 |
| Fetal Skeletal Muscle | 5.2 | 46.7 | 19.1 | 19.2 |
| Skeletal Muscle Pool | 9.2 | 24.7 | 22.1 | 22.7 |
| Spleen Pool | 0.0 | 2.4 | 2.7 | 0.6 |
| Thymus Pool | 2.0 | 18.4 | 7.7 | 3.1 |
| CNS cancer (glio/astro) | 1.5 | 5.8 | 10.9 | 2.2 |
| U87-MG | | | | |
| CNS cancer (glio/astro) | 0.3 | 1.5 | 3.8 | 0.8 |
| U-118-MG | | | | |
| CNS cancer | 0.0 | 0.7 | 1.4 | 0.5 |
| (neuro; met) SK-N-AS | | | | |
| CNS cancer (astro) SF- | 0.0 | 0.2 | 0.1 | 0.2 |
| 539 | | | | |
| CNS cancer (astro) | 1.1 | 3.1 | 11.7 | 2.8 |
| SNB-75 | | | | |
| CNS cancer (glio) | 79.0 | 12.8 | 100.0 | 97.9 |
| SNB-19 | | | | |
| CNS cancer (glio) SF- | 0.0 | 0.0 | 8.2 | 1.5 |
| 295 | | | | |
| Brain (Amygdala) Pool | 0.8 | 7.9 | 8.0 | 4.4 |
| Brain (cerebellum) | 0.4 | 1.8 | 8.8 | 1.2 |
| Brain (fetal) | 0.7 | 8.4 | 6.8 | 2.1 |
| Brain (Hippocampus) | 3.2 | 9.9 | 11.0 | 4.3 |
| Pool | | | | |
| Cerebral Cortex Pool | 0.6 | 1.8 | 11.6 | 2.0 |
| Brain (Substantia nigra) | 2.2 | 4.2 | 10.0 | 2.0 |
| Pool | | | | |
| Brain (Thalamus) Pool | 2.7 | 9.1 | 9.7 | 2.8 |
| Brain (whole) | 0.4 | 3.3 | 5.6 | 1.9 |
| Spinal Cord Pool | 2.3 | 13.1 | 12.2 | 4.2 |
| Adrenal Gland | 0.3 | 7.4 | 4.8 | 0.9 |
| Pituitary gland Pool | 0.0 | 1.8 | 1.4 | 0.6 |
| Salivary Gland | 0.0 | 2.3 | 1.1 | 0.0 |
| Thyroid (female) | 0.3 | 3.3 | 1.9 | 1.3 |
| Pancreatic ca. | 0.0 | 0.5 | 0.7 | 0.6 |
| CAPAN2 | | | | |
| Pancreas Pool | 0.0 | 3.5 | 3.2 | 1.0 |

TABLE AQH
Panel 4.1D

| | Rel. Exp. (%) | Rel. Exp. (%) | Rel. Exp. (%) |
|---|---|---|---|
| | Ag6425, Run | Ag6435, Run | Ag6439, Run |
| Tissue Name | 268713999 | 268713480 | 268760823 |
| Secondary Th1 act | 0.0 | 0.0 | 0.0 |
| Secondary Th2 act | 0.0 | 0.0 | 0.0 |
| Secondary Tr1 act | 0.0 | 0.0 | 0.0 |
| Secondary Th1 rest | 0.0 | 0.0 | 0.0 |
| Secondary Th2 rest | 0.0 | 0.7 | 0.0 |
| Secondary Tr1 rest | 0.0 | 0.0 | 0.0 |
| Primary Th1 act | 0.0 | 0.0 | 0.0 |
| Primary Th2 act | 0.0 | 0.7 | 0.0 |
| Primary Tr1 act | 0.0 | 0.0 | 0.0 |
| Primary Th1 rest | 0.0 | 0.0 | 1.2 |
| Primary Th2 rest | 0.0 | 0.0 | 0.0 |
| Primary Tr1 rest | 0.0 | 0.0 | 0.0 |
| CD45RA CD4 lymphocyte act | 0.0 | 0.8 | 2.6 |
| CD45RO CD4 lymphocyte act | 0.0 | 1.6 | 2.3 |
| CD8 lymphocyte act | 0.0 | 0.0 | 0.0 |
| Secondary CD8 lymphocyte | 0.0 | 0.0 | 0.0 |
| rest | | | |
| Secondary CD8 lymphocyte | 0.0 | 0.0 | 0.0 |
| act | | | |
| CD4 lymphocyte none | 0.0 | 0.0 | 0.0 |
| 2ry Th1/Th2/Tr1_anti-CD95 | 0.0 | 0.0 | 1.2 |
| CH11 | | | |
| LAK cells rest | 2.7 | 6.1 | 15.2 |
| LAK cells IL-2 | 0.0 | 0.0 | 0.0 |
| LAK cells IL-2 + IL-12 | 0.0 | 0.0 | 0.0 |
| LAK cells IL-2 + IFN gamma | 0.0 | 0.0 | 0.0 |
| LAK cells IL-2 + IL-18 | 0.0 | 0.0 | 0.0 |
| LAK cells PMA/ionomycin | 15.7 | 6.1 | 9.0 |
| NK Cells IL-2 rest | 0.0 | 0.0 | 1.4 |
| Two Way MLR 3 day | 0.0 | 0.9 | 1.4 |
| Two Way MLR 5 day | 0.0 | 0.0 | 0.0 |
| Two Way MLR 7 day | 13.2 | 2.9 | 3.7 |
| PBMC rest | 0.0 | 0.0 | 0.0 |
| PBMC PWM | 0.0 | 0.0 | 0.0 |
| PBMC PHA-L | 0.0 | 0.0 | 0.0 |
| Ramos (B cell) none | 0.0 | 0.0 | 0.0 |
| Ramos (B cell) ionomycin | 0.0 | 0.0 | 0.0 |
| B lymphocytes PWM | 0.0 | 0.0 | 0.0 |
| B lymphocytes CD40L and IL- | 0.0 | 0.0 | 0.0 |
| 4 | | | |
| EOL-1 dbcAMP | 9.1 | 0.0 | 68.8 |
| EOL-1 dbcAMP | 0.0 | 1.0 | 1.8 |
| PMA/ionomycin | | | |
| Dendritic cells none | 13.8 | 0.7 | 0.0 |
| Dendritic cells LPS | 0.0 | 0.0 | 0.0 |
| Dendritic cells anti-CD40 | 3.3 | 1.6 | 0.0 |
| Monocytes rest | 0.0 | 0.0 | 0.0 |
| Monocytes LPS | 0.0 | 0.0 | 2.6 |
| Macrophages rest | 0.0 | 0.0 | 0.0 |
| Macrophages LPS | 0.0 | 0.8 | 9.2 |
| HUVEC none | 0.0 | 0.0 | 0.0 |
| HUVEC starved | 0.0 | 0.0 | 0.0 |
| HUVEC IL-1beta | 0.0 | 0.0 | 0.0 |
| HUVEC IFN gamma | 0.0 | 0.0 | 0.0 |
| HUVEC TNF alpha + IFN | 0.0 | 0.6 | 0.0 |
| gamma | | | |
| HUVEC TNF alpha + IL4 | 0.0 | 0.0 | 0.0 |
| HUVEC IL-11 | 0.0 | 0.0 | 0.0 |
| Lung Microvascular EC none | 0.0 | 0.0 | 0.0 |
| Lung Microvascular EC | 0.0 | 0.0 | 0.0 |
| TNFalpha + IL-1beta | | | |
| Microvascular Dermal EC | 0.0 | 0.0 | 0.0 |
| none | | | |
| Microsvasular Dermal EC | 0.0 | 0.0 | 0.0 |
| TNFalpha + IL-1beta | | | |
| Bronchial epithelium | 0.0 | 0.0 | 0.0 |
| TNFalpha + IL1beta | | | |
| Small airway epithelium none | 0.0 | 0.0 | 0.0 |
| Small airway epithelium | 0.0 | 0.0 | 0.0 |
| TNFalpha + IL-1beta | | | |
| Coronery artery SMC rest | 0.0 | 0.5 | 0.0 |
| Coronery artery SMC | 6.2 | 0.0 | 0.0 |
| TNFalpha + IL-1beta | | | |
| Astrocytes rest | 100.0 | 100.0 | 100.0 |
| Astrocytes TNFalpha + IL- | 74.2 | 97.9 | 95.9 |
| 1beta | | | |
| KU-812 (Basophil) rest | 0.0 | 0.0 | 0.0 |
| KU-812 (Basophil) | 0.0 | 0.0 | 0.0 |
| PMA/ionomycin | | | |
| CCD1106 (Keratinocytes) none | 0.0 | 0.0 | 0.0 |
| CCD1106 (Keratinocytes) | 0.0 | 0.0 | 0.0 |
| TNFalpha + IL-1beta | | | |
| Liver cirrhosis | 4.6 | 5.1 | 8.5 |
| NC1-H292 none | 0.0 | 0.0 | 0.0 |
| NCI-H292 IL-4 | 0.0 | 0.0 | 0.0 |
| NCI-H292 IL-9 | 0.0 | 0.0 | 0.0 |
| NCI-H292 IL-13 | 0.0 | 0.0 | 0.0 |
| NCI-H292 IFN gamma | 0.0 | 0.0 | 0.0 |
| HPAEC none | 0.0 | 0.0 | 0.0 |
| HPAEC TNF alpha + IL-1 beta | 0.0 | 0.0 | 0.0 |
| Lung fibroblast none | 31.4 | 62.9 | 94.0 |
| Lung fibroblast TNF alpha + | 22.2 | 25.2 | 62.9 |
| IL-1 beta | | | |
| Lung fibroblast IL-4 | 19.1 | 23.3 | 34.9 |
| Lung fibroblast IL-9 | 23.5 | 20.4 | 96.6 |
| Lung fibroblast IL-13 | 4.5 | 15.0 | 13.4 |
| Lung fibroblast IFN gamma | 15.7 | 29.9 | 89.5 |
| Dermal fibroblast CCD1070 | 0.0 | 5.6 | 4.1 |
| rest | | | |
| Dermal fibroblast CCD1070 | 0.0 | 0.8 | 2.3 |
| TNF alpha | | | |
| Dermal fibroblast CCD1070 | 0.0 | 0.7 | 0.0 |
| IL-1 beta | | | |
| Dermal fibroblast IFN gamma | 8.5 | 20.0 | 26.6 |
| Dermal fibroblast IL-4 | 4.1 | 22.7 | 25.5 |
| Dermal Fibroblasts rest | 8.0 | 20.7 | 47.3 |
| Neutrophils TNFa + LPS | 0.0 | 1.2 | 0.0 |
| Neutrophils rest | 0.0 | 0.0 | 0.0 |
| Colon | 4.0 | 7.9 | 8.4 |
| Lung | 0.0 | 1.6 | 2.1 |
| Thymus | 0.0 | 2.0 | 2.4 |
| Kidney | 4.9 | 10.2 | 5.2 |

CNS_neurodegeneration_v1.0 Summary: Ag6435/Ag6439/Ag6440 This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of the of this gene in treatment of central nervous system disorders.

General_screening_panel_v1.6 Summary: Ag6425/Ag6435/Ag6439/Ag6440 Highest expression of this gene is detected in kidney, ovarian cancer IGROV-1 cell line and brain cancer SNB-19 cell lines (CTs=28-31). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Panel 4.1D Summary: Ag6425/Ag6435/Ag6439 Highest expression of this gene is detected in both resting and cytokine activated astrocytes (CTs=22-34.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AR. CG56054-10 and CG56054-11: Integrin Alpha 7-Like Protein.

Expression of gene CG56054-10 and CG56054-11 was assessed using the primer-probe sets Ag4983, Ag6442, Ag6425, Ag6428, Ag6431, Ag6433, Ag6435, Ag6440, Ag6446, Ag6447, Ag6413 and Ag6964, described in Tables ARA, ARB, ARC, ARD, ARE, ARF, ARG, ARH, ARI, ARJ, ARK and ARL. Results of the RTQ-PCR runs are shown in Tables ARM, ARN, ARO, ARP, ARQ and ARR. Note Ag6433 is specific for CG56054-11. Also, the CG56054-11 gene is only recognized by probe-primer sets Ag6433, Ag6431, Ag6446 and Ag6964.

TABLE ARA

uz,18/29 Probe Name Ag4983

| | | | Start | |
|---|---|---|---|---|
| Primers | Sequences | Length | Position | SEQ ID No |
| Forward | 5′- ccaggtcaccttctacctcatc-3′ | 22 | 2342 | 534 |
| Probe | TET-5′-cttagcacctccgggatcagcatt-3′-TAMRA | 24 | 2364 | 535 |
| Reverse | 5′-aacagcagctctacctccagtt-3′ | 22 | 2398 | 536 |

TABLE ARB

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3′ | 22 | 2781 | 537 |
| Probe | TET-5′-ccacctgagcagcaggagcct-3′-TAMRA | 21 | 2820 | 538 |
| Reverse | 5′-gcgcagtccagggtg-3′ | 15 | 2906 | 539 |

TABLE ARC

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 3516 | 540 |
| Probe | TET-5′-catcccgagctgggcccc-3′-TAMRA | 18 | 3548 | 541 |
| Reverse | 5′-gccctggatgcccat-3′ | 15 | 3567 | 542 |

TABLE ARD

Probe Name Ag6428

| | | | | |
|---|---|---|---|---|
| Primers | Sequences | Length | Start | SEQ ID No |

| | | | | |
|---|---|---|---|---|
| Forward | 5′-cttcatctaccatgggagca-3′ | 20 | 1301 | 543 |
| Probe | TET-5′-ccttcacaggtgctggagggc-3′-TAMRA | 21 | 1341 | 544 |
| Reverse | 5′-agggagtagccgaagctct-3′ | 19 | 1378 | 545 |

TABLE ARE

Probe Name Ag6431

| | | | Start | |
|---|---|---|---|---|
| Primers | Sequences | Length | Position | SEQ ID No |
| Forward | 5′-aaacatcaccctggactgc-3′ | 19 | 2900 | 546 |
| Probe | TET-5′-tggtgttcagctgcccactctacag-3′-TAMRA | 25 | 2941 | 547 |
| Reverse | 5′-ccgcgcggtcaaa-3′ | 13 | 2967 | 548 |

TABLE ARF

Probe Name Ag6433

| | | | Start | |
|---|---|---|---|---|
| Primers | Sequences | Length | Position | SEQ ID No |
| Primers | 5′-ggagtcagtgtcctctgctga-3′ | 21 | 534 | 549 |
| Probe | TET-5′-ctgcccactctacagctttgaccgc-3′-TAMRA | 25 | 615 | 550 |
| Reverse | 5′-cccagacatgcagcacag-3′ | 18 | 644 | 551 |

TABLE ARG

Probe Name Ag6435

| | | | | |
|---|---|---|---|---|
| | | | | SEQ |
| | | | Start | ID |
| Primers | Sequences | Length | Position | No |

| | | | | |
|---|---|---|---|---|
| Forward | 5′-ggccagggtggagct-3′ | 15 | 731 | 552 |
| Probe | TET-5′- | 21 | 766 | 553 |
| | acctggcacacctggacgacg- | | | |
| | 3′-TAMRA | | | |
| Reverse | 5′-cagggaccgggatga-3′ | 15 | 829 | 554 |

TABLE ARH

Probe Name Ag6440

| | | | | |
|---|---|---|---|---|
| | | | | SEQ |
| | | | Start | ID |
| Primers | Sequences | Length | Position | No |

| | | | | |
|---|---|---|---|---|
| Forward | 5′-accatcctgaggaacaact | 20 | 3473 | 555 |
| | g-3′ | | | |
| Probe | TET-5′- | 19 | 3540 | 556 |
| | ctgacgggcatcccgagct | | | |
| | -3′-TAMRA | | | |
| Reverse | 5′-ccctggatgcccatc-3′ | 15 | 3566 | 557 |

TABLE ARI

Probe Name Ag6446

| | | | | |
|---|---|---|---|---|
| | | | Start | |
| Primers | Sequences | Length | Position | SEQ ID No |

| | | | | |
|---|---|---|---|---|
| Forward | 5′-gcttcttccatcggagca-3′ | 18 | 3256 | 558 |
| Probe | TET-5′-caactatcaccgggcctgtctggc-3′-TAMRA | 24 | 3296 | 559 |
| Reverse | 5′-catggctgaaggctgca-3′ | 17 | 3322 | 560 |

TABLE ARJ

Probe Name Ag6447

| | | | | |
|---|---|---|---|---|
| | | | Start | |
| Primers | Sequences | Length | Position | SEQ ID No |

| | | | | |
|---|---|---|---|---|
| Forward | 5′-gacgacggtccctacga-3′ | 17 | 780 | 561 |
| Probe | TET-5′-tcatcccggtccctgccaa-3′-TAMRA | 19 | 829 | 562 |
| Reverse | 5′-gtcaatagagaagccaaagtagct-3′ | 24 | 849 | 563 |

TABLE ARK

Probe Name Ag6413

| | | | Start | |
|---|---|---|---|---|
| Primers | Sequences | Length | Position | SEQ ID No |
| Forward | 5′-ggtgaagacaagatctgccag-3′ | 21 | 1980 | 564 |
| Probe | TET-5′-tgtacccgggtcagcgacacg-3′-TAMRA | 21 | 2031 | 565 |
| Reverse | 5′-gctgttgttccatccacatc-3′ | 20 | 2073 | 566 |

TABLE ARL

Probe Name Ag6964

| | | | | |
|---|---|---|---|---|
| | | | Start | |
| Primers | Sequences | Length | Position | SEQ ID No |

| | | | | |
|---|---|---|---|---|
| Forward | 5′-ggccccagacatgca-3′ | 15 | 2986 | 567 |
| Probe | TET-5′- | 23 | 2957 | 568 |
| | actctacagctttgaccgcgcgg-3′-TAMRA | | | |
| Reverse | 5′-gccaactgtgtggtgttca-3′ | 19 | 2931 | 569 |

TABLE ARM
CNS_neurodegeneration_v1.0

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag4983, | Ag6413, | Ag6428, | Ag6431, | Ag6433, |
| Tissue | Run | Run | Run | Run | Run |
| Name | 218649223 | 269253983 | 266937081 | 268030722 | 268030711 |
| AD 1 | 23.7 | 24.8 | 18.0 | 18.8 | 27.0 |
| Hippo | | | | | |
| AD 2 | 41.2 | 52.9 | 32.3 | 28.7 | 43.5 |
| Hippo | | | | | |
| AD 3 | 8.9 | 6.4 | 3.7 | 7.5 | 9.9 |
| Hippo | | | | | |
| AD 4 | 14.8 | 25.5 | 10.7 | 18.8 | 18.2 |
| Hippo | | | | | |
| AD 5 | 44.8 | 41.8 | 53.2 | 38.4 | 44.8 |
| Hippo | | | | | |
| AD 6 | 100.0 | 100.0 | 100.0 | 100.0 | 100.0 |
| Hippo | | | | | |
| Control | 24.3 | 36.1 | 18.7 | 29.5 | 20.7 |
| 2 Hippo | | | | | |
| Control | 42.9 | 43.8 | 27.0 | 32.3 | 52.1 |
| 4 Hippo | | | | | |
| Control | 14.2 | 11.4 | 4.6 | 6.0 | 6.8 |
| (Path) 3 | | | | | |
| Hippo | | | | | |
| AD 1 | 23.3 | 15.9 | 12.9 | 17.1 | 23.7 |
| Temporal | | | | | |
| Ctx | | | | | |
| AD 2 | 41.5 | 47.3 | 31.0 | 39.8 | 24.7 |
| Temporal | | | | | |
| Ctx | | | | | |
| AD 3 | 9.5 | 9.8 | 6.0 | 11.3 | 11.5 |
| Temporal | | | | | |
| Ctx | | | | | |
| AD 4 | 30.6 | 39.0 | 20.2 | 25.3 | 19.1 |
| Temporal | | | | | |
| Ctx | | | | | |
| AD 5 | 45.4 | 37.1 | 39.2 | 36.3 | 30.6 |
| Inf | | | | | |
| Temporal | | | | | |
| Ctx | | | | | |
| AD 5 | 51.1 | 39.0 | 42.0 | 32.3 | 38.7 |
| Sup | | | | | |
| Temporal | | | | | |
| Ctx | | | | | |
| AD 6 | 38.2 | 59.9 | 49.3 | 46.7 | 43.5 |
| Inf | | | | | |
| Temporal | | | | | |
| Ctx | | | | | |
| AD 6 | 43.8 | 48.6 | 48.3 | 50.3 | 62.0 |
| Sup | | | | | |
| Temporal | | | | | |
| Ctx | | | | | |
| Control | 12.2 | 23.0 | 12.9 | 15.6 | 11.8 |
| 1 | | | | | |
| Temporal | | | | | |
| Ctx | | | | | |
| Control | 14.2 | 32.5 | 18.2 | 17.4 | 31.0 |
| 2 | | | | | |
| Temporal | | | | | |
| Ctx | | | | | |
| Control | 15.1 | 15.3 | 9.6 | 14.5 | 12.6 |
| 3 | | | | | |
| Temporal | | | | | |
| Ctx | | | | | |
| Control | 23.7 | 25.0 | 15.2 | 13.1 | 17.7 |
| 3 | | | | | |
| Temporal | | | | | |
| Ctx | | | | | |
| Control | 26.1 | 47.0 | 27.0 | 30.6 | 39.2 |
| (Path) 1 | | | | | |
| Temporal | | | | | |
| Ctx | | | | | |
| Control | 24.5 | 25.9 | 16.0 | 20.4 | 14.2 |
| (Path) 2 | | | | | |
| Temporal | | | | | |
| Ctx | | | | | |
| Control | 11.7 | 16.0 | 7.5 | 10.9 | 13.6 |
| (Path) 3 | | | | | |
| Temporal | | | | | |
| Ctx | | | | | |
| Control | 21.9 | 27.4 | 17.1 | 18.2 | 11.9 |
| (Path) 4 | | | | | |
| Temporal | | | | | |
| Ctx | | | | | |
| AD 1 | 16.0 | 11.9 | 10.2 | 11.5 | 13.1 |
| Occipital | | | | | |
| Ctx | | | | | |
| AD 2 | 0.0 | 0.0 | 0.0 | 0.0 | 0.0 |
| Occipital | | | | | |
| Ctx | | | | | |
| (Missing) | | | | | |
| AD 3 | 10.7 | 6.0 | 6.4 | 8.8 | 8.0 |
| Occipital | | | | | |
| Ctx | | | | | |
| AD 4 | 18.9 | 23.7 | 13.0 | 17.9 | 15.9 |
| Occipital | | | | | |
| Ctx | | | | | |
| AD 5 | 24.8 | 28.3 | 25.3 | 22.5 | 36.6 |
| Occipital | | | | | |
| Ctx | | | | | |
| AD 6 | 20.6 | 31.9 | 20.2 | 17.0 | 16.0 |
| Occipital | | | | | |
| Ctx | | | | | |
| Control | 9.5 | 14.4 | 6.0 | 8.7 | 15.1 |
| 1 | | | | | |
| Occipital | | | | | |
| Ctx | | | | | |
| Control | 31.9 | 42.6 | 26.4 | 33.2 | 25.0 |
| 2 | | | | | |
| Occipital | | | | | |
| Ctx | | | | | |
| Control | 18.8 | 13.0 | 10.7 | 17.1 | 12.1 |
| 3 | | | | | |
| Occipital | | | | | |
| Ctx | | | | | |
| Control | 18.2 | 17.0 | 12.0 | 12.6 | 14.4 |
| 4 | | | | | |
| Occipital | | | | | |
| Ctx | | | | | |
| Control | 38.2 | 52.5 | 35.6 | 36.1 | 40.9 |
| (Path) 1 | | | | | |
| Occipital | | | | | |
| Ctx | | | | | |
| Control | 9.6 | 14.1 | 6.7 | 7.9 | 6.3 |
| (Path) 2 | | | | | |
| Occipital | | | | | |
| Ctx | | | | | |
| Control | 4.8 | 8.7 | 5.4 | 6.0 | 5.3 |
| (Path) 3 | | | | | |
| Occipital | | | | | |
| Ctx | | | | | |
| Control | 16.2 | 13.2 | 13.2 | 10.2 | 5.8 |
| (Path) 4 | | | | | |
| Occipital | | | | | |
| Ctx | | | | | |
| Control | 14.4 | 21.9 | 8.8 | 16.3 | 13.2 |
| 1 | | | | | |
| Parietal | | | | | |
| Ctx | | | | | |
| Control | 32.8 | 28.9 | 34.4 | 28.3 | 27.4 |
| 2 | | | | | |
| Parietal | | | | | |
| Ctx | | | | | |
| Control | 20.6 | 19.8 | 11.5 | 8.7 | 18.2 |
| 3 | | | | | |
| Parietal | | | | | |
| Ctx | | | | | |
| Control | 35.4 | 62.4 | 34.2 | 39.2 | 44.1 |
| (Path) 1 | | | | | |
| Parietal | | | | | |
| Ctx | | | | | |
| Control | 22.1 | 23.8 | 19.6 | 22.5 | 16.5 |
| (Path) 2 | | | | | |
| Parietal | | | | | |
| Ctx | | | | | |
| Control | 11.2 | 15.4 | 3.9 | 7.1 | 8.7 |
| (Path) 3 | | | | | |
| Parietal | | | | | |
| Ctx | | | | | |
| Control | 31.2 | 34.2 | 24.8 | 8.8 | 14.0 |
| (Path) 4 | | | | | |
| Parietal | | | | | |
| Ctx | | | | | |
| | Rel. | Rel. | Rel. | Rel. | Rel. |
| | Exp. (%) | Exp. (%) | Exp. (%) | Exp. (%) | Exp. (%) |
| | Ag6435, | Ag6440, | Ag6442, | Ag6446, | Ag6447, |
| Tissue | Run | Run | Run | Run | Run |
| Name | 269253997 | 269254003 | 264979298 | 269254006 | 269254007 |
| AD 1 | 17.1 | 18.9 | 19.2 | 42.9 | 18.8 |
| Hippo | | | | | |
| AD 2 | 27.9 | 61.1 | 49.7 | 41.8 | 10.4 |
| Hippo | | | | | |
| AD 3 | 4.8 | 9.7 | 20.4 | 23.7 | 0.0 |
| Hippo | | | | | |
| AD 4 | 18.3 | 23.3 | 5.6 | 29.9 | 4.6 |
| Hippo | | | | | |
| AD 5 | 46.7 | 34.6 | 57.4 | 67.8 | 11.0 |
| Hippo | | | | | |
| AD 6 | 100.0 | 100.0 | 90.1 | 100.0 | 100.0 |
| Hippo | | | | | |
| Control | 8.5 | 29.9 | 28.5 | 39.2 | 3.1 |
| 2 Hippo | | | | | |
| Control | 29.9 | 54.7 | 86.5 | 62.4 | 43.8 |
| 4 Hippo | | | | | |
| Control | 5.2 | 5.8 | 0.0 | 14.6 | 5.3 |
| (Path) 3 | | | | | |
| Hippo | | | | | |
| AD 1 | 12.8 | 12.6 | 16.8 | 72.7 | 9.0 |
| Temporal | | | | | |
| Ctx | | | | | |
| AD 2 | 45.1 | 59.0 | 21.6 | 43.2 | 21.0 |
| Temporal | | | | | |
| Ctx | | | | | |
| AD 3 | 4.1 | 17.1 | 5.7 | 36.3 | 3.9 |
| Temporal | | | | | |
| Ctx | | | | | |
| AD 4 | 6.8 | 29.9 | 8.7 | 43.2 | 7.7 |
| Temporal | | | | | |
| Ctx | | | | | |
| AD 5 | 1.6 | 41.8 | 73.7 | 63.3 | 23.7 |
| Inf | | | | | |
| Temporal | | | | | |
| Ctx | | | | | |
| AD 5 | 33.2 | 39.2 | 55.9 | 95.3 | 11.4 |
| Sup | | | | | |
| Temporal | | | | | |
| Ctx | | | | | |
| AD 6 | 52.1 | 48.6 | 76.8 | 45.1 | 88.9 |
| Inf | | | | | |
| Temporal | | | | | |
| Ctx | | | | | |
| AD 6 | 37.6 | 17.0 | 59.9 | 30.6 | 61.1 |
| Sup | | | | | |
| Temporal | | | | | |
| Ctx | | | | | |
| Control | 6.7 | 23.3 | 46.7 | 5.9 | 2.8 |
| 1 | | | | | |
| Temporal | | | | | |
| Ctx | | | | | |
| Control | 7.3 | 43.5 | 50.0 | 13.6 | 16.0 |
| 2 | | | | | |
| Temporal | | | | | |
| Ctx | | | | | |
| Control | 4.4 | 9.2 | 9.5 | 12.5 | 3.1 |
| 3 | | | | | |
| Temporal | | | | | |
| Ctx | | | | | |
| Control | 11.7 | 30.1 | 13.6 | 26.6 | 13.6 |
| 3 | | | | | |
| Temporal | | | | | |
| Ctx | | | | | |
| Control | 24.8 | 51.1 | 46.0 | 21.2 | 13.8 |
| (Path) 1 | | | | | |
| Temporal | | | | | |
| Ctx | | | | | |
| Control | 9.8 | 7.2 | 10.0 | 27.2 | 2.6 |
| (Path) 2 | | | | | |
| Temporal | | | | | |
| Ctx | | | | | |
| Control | 3.5 | 9.9 | 31.0 | 24.5 | 6.3 |
| (Path) 3 | | | | | |
| Temporal | | | | | |
| Ctx | | | | | |
| Control | 14.8 | 14.9 | 39.5 | 19.2 | 7.0 |
| (Path) 4 | | | | | |
| Temporal | | | | | |
| Ctx | | | | | |
| AD 1 | 15.0 | 5.8 | 6.3 | 39.5 | 0.0 |
| Occipital | | | | | |
| Ctx | | | | | |
| AD 2 | 0.0 | 0.0 | 0.0 | 0.0 | 0.0 |
| Occipital | | | | | |
| Ctx | | | | | |
| (Missing) | | | | | |
| AD 3 | 8.0 | 7.8 | 4.9 | 19.3 | 0.0 |
| Occipital | | | | | |
| Ctx | | | | | |
| AD 4 | 6.8 | 35.4 | 11.1 | 25.3 | 3.5 |
| Occipital | | | | | |
| Ctx | | | | | |
| AD 5 | 12.7 | 16.6 | 42.3 | 25.2 | 3.8 |
| Occipital | | | | | |
| Ctx | | | | | |
| AD 6 | 5.9 | 23.5 | 14.8 | 9.7 | 8.5 |
| Occipital | | | | | |
| Ctx | | | | | |
| Control | 4.1 | 15.2 | 8.8 | 6.5 | 1.3 |
| 1 | | | | | |
| Occipital | | | | | |
| Ctx | | | | | |
| Control | 20.3 | 35.8 | 82.4 | 8.1 | 13.7 |
| 2 | | | | | |
| Occipital | | | | | |
| Ctx | | | | | |
| Control | 7.5 | 4.4 | 8.8 | 15.8 | 5.0 |
| 3 | | | | | |
| Occipital | | | | | |
| Ctx | | | | | |
| Control | 3.3 | 12.9 | 24.0 | 23.3 | 1.3 |
| 4 | | | | | |
| Occipital | | | | | |
| Ctx | | | | | |
| Control | 25.9 | 22.4 | 100.0 | 23.3 | 12.1 |
| (Path) 1 | | | | | |
| Occipital | | | | | |
| Ctx | | | | | |
| Control | 7.4 | 5.0 | 9.3 | 15.6 | 13.2 |
| (Path) 2 | | | | | |
| Occipital | | | | | |
| Ctx | | | | | |
| Control | 2.3 | 6.7 | 4.1 | 4.5 | 9.4 |
| (Path) 3 | | | | | |
| Occipital | | | | | |
| Ctx | | | | | |
| Control | 21.0 | 11.9 | 32.8 | 5.9 | 20.4 |
| (Path) 4 | | | | | |
| Occipital | | | | | |
| Ctx | | | | | |
| Control | 12.5 | 33.2 | 9.2 | 5.7 | 5.0 |
| 1 | | | | | |
| Parietal | | | | | |
| Ctx | | | | | |
| Control | 41.2 | 17.4 | 28.1 | 74.2 | 25.5 |
| 2 | | | | | |
| Parietal | | | | | |
| Ctx | | | | | |
| Control | 13.2 | 21.6 | 9.1 | 8.6 | 16.7 |
| 3 | | | | | |
| Parietal | | | | | |
| Ctx | | | | | |
| Control | 22.5 | 47.3 | 69.3 | 24.0 | 4.2 |
| (Path) 1 | | | | | |
| Parietal | | | | | |
| Ctx | | | | | |
| Control | 26.8 | 17.1 | 37.6 | 23.7 | 14.4 |
| (Path) 2 | | | | | |
| Parietal | | | | | |
| Ctx | | | | | |
| Control | 7.5 | 11.7 | 10.4 | 11.0 | 5.9 |
| (Path) 3 | | | | | |
| Parietal | | | | | |
| Ctx | | | | | |
| Control | 20.6 | 29.3 | 27.5 | 27.0 | 9.4 |
| (Path) 4 | | | | | |
| Parietal | | | | | |
| Ctx | | | | | |

TABLE ARN
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 13.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro; met) SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE ARO
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro; met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE ARP
General_screening_panel_v1.6

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag6413, | Ag6425, | Ag6428, | Ag6431, | Ag6431, |
| Tissue | Run | Run | Run | Run | Run |
| Name | 277249371 | 277221721 | 277222439 | 277633568 | 278389390 |
| Adipose | 25.9 | 2.6 | 20.0 | 17.4 | 13.8 |
| Melanoma* | 0.5 | 0.0 | 2.0 | 0.8 | 0.9 |
| Hs688(A).T | | | | | |
| Melanoma* | 2.7 | 0.2 | 4.1 | 2.5 | 2.2 |
| Hs688(B).T | | | | | |
| Melanoma* | 0.3 | 0.0 | 0.7 | 0.4 | 0.4 |
| M14 | | | | | |
| Melanoma* | 0.0 | 0.0 | 0.1 | 0.0 | 0.0 |
| LOXIMVI | | | | | |
| Melanoma* | 15.2 | 2.2 | 30.4 | 18.2 | 14.6 |
| SK-MEL-5 | | | | | |
| Squamous | 0.0 | 0.0 | 0.1 | 0.1 | 0.2 |
| cell | | | | | |
| carcinoma | | | | | |
| SCC-4 | | | | | |
| Testis | 5.2 | 3.5 | 8.8 | 10.4 | 9.0 |
| Pool | | | | | |
| Prostate | 1.9 | 0.5 | 2.5 | 1.9 | 1.8 |
| ca.* | | | | | |
| (bone | | | | | |
| met) PC- | | | | | |
| 3 | | | | | |
| Prostate | 8.1 | 1.0 | 11.5 | 11.3 | 12.1 |
| Pool | | | | | |
| Placenta | 0.5 | 0.0 | 0.7 | 0.1 | 0.1 |
| Uterus | 2.2 | 1.5 | 4.5 | 4.6 | 4.5 |
| Pool | | | | | |
| Ovarian | 0.9 | 0.3 | 1.1 | 0.7 | 1.1 |
| ca. | | | | | |
| OVCAR-3 | | | | | |
| Ovarian | 0.8 | 0.2 | 1.7 | 0.8 | 0.9 |
| ca. SK- | | | | | |
| OV-3 | | | | | |
| Ovarian | 0.2 | 0.0 | 0.9 | 0.4 | 0.8 |
| ca. | | | | | |
| OVCAR-4 | | | | | |
| Ovarian | 1.6 | 1.3 | 2.9 | 1.3 | 1.7 |
| ca. | | | | | |
| OVCAR-5 | | | | | |
| Ovarian | 100.0 | 100.0 | 77.9 | 84.7 | 97.9 |
| ca. | | | | | |
| IGROV-1 | | | | | |
| Ovarian | 13.6 | 21.9 | 14.0 | 15.6 | 14.6 |
| ca. | | | | | |
| OVCAR-8 | | | | | |
| Ovary | 2.7 | 0.3 | 5.2 | 3.1 | 2.3 |
| Breast | 0.3 | 0.0 | 0.3 | 0.1 | 0.2 |
| ca. MCF- | | | | | |
| 7 | | | | | |
| Breast | 0.1 | 0.0 | 0.4 | 0.2 | 0.2 |
| ca. | | | | | |
| MDA- | | | | | |
| MB-231 | | | | | |
| Breast | 0.5 | 0.0 | 0.5 | 0.1 | 0.5 |
| ca. BT | | | | | |
| 549 | | | | | |
| Breast | 0.0 | 0.0 | 0.5 | 0.2 | 0.3 |
| ca. T47D | | | | | |
| Breast | 0.6 | 0.0 | 0.7 | 0.6 | 0.6 |
| ca. | | | | | |
| MDA-N | | | | | |
| Breast | 15.0 | 4.1 | 21.8 | 14.6 | 10.7 |
| Pool | | | | | |
| Trachea | 4.5 | 0.7 | 8.4 | 4.8 | 4.2 |
| Lung | 2.8 | 0.7 | 2.3 | 4.2 | 3.2 |
| Fetal | 3.9 | 0.3 | 9.1 | 5.0 | 4.8 |
| Lung | | | | | |
| Lung ca. | 2.0 | 0.9 | 3.5 | 3.3 | 2.6 |
| NCI- | | | | | |
| N417 | | | | | |
| Lung ca. | 3.5 | 2.7 | 6.5 | 5.0 | 3.5 |
| LX-1 | | | | | |
| Lung ca. | 0.1 | 0.0 | 0.3 | 0.1 | 0.2 |
| NCI- | | | | | |
| H146 | | | | | |
| Lung ca. | 4.0 | 0.4 | 6.8 | 5.3 | 4.5 |
| SHP-77 | | | | | |
| Lung ca. | 0.3 | 2.6 | 0.9 | 0.0 | 0.4 |
| A549 | | | | | |
| Lung ca. | 0.2 | 0.0 | 0.9 | 0.6 | 0.3 |
| NCI- | | | | | |
| H526 | | | | | |
| Lung ca. | 2.9 | 1.0 | 4.6 | 4.8 | 3.2 |
| NCI-H23 | | | | | |
| Lung ca. | 0.0 | 0.0 | 0.2 | 0.1 | 0.3 |
| NCI- | | | | | |
| H460 | | | | | |
| Lung ca. | 0.5 | 0.0 | 0.5 | 1.0 | 0.6 |
| HOP-62 | | | | | |
| Lung ca. | 1.7 | 0.6 | 2.3 | 1.7 | 1.3 |
| NCI- | | | | | |
| H522 | | | | | |
| Liver | 0.1 | 0.0 | 0.0 | 0.0 | 0.0 |
| Fetal | 0.3 | 0.3 | 1.1 | 0.6 | 0.5 |
| Liver | | | | | |
| Liver ca. | 0.1 | 0.3 | 0.2 | 0.0 | 0.2 |
| HepG2 | | | | | |
| Kidney | 27.9 | 0.0 | 47.0 | 33.9 | 28.1 |
| Pool | | | | | |
| Fetal | 1.4 | 0.0 | 4.9 | 4.1 | 4.0 |
| Kidney | | | | | |
| Renal ca. | 0.2 | 0.0 | 0.2 | 0.3 | 0.1 |
| 786-0 | | | | | |
| Renal ca. | 0.0 | 1.8 | 0.2 | 0.0 | 0.3 |
| A498 | | | | | |
| Renal ca. | 1.5 | 0.5 | 2.5 | 1.7 | 1.5 |
| ACHN | | | | | |
| Renal ca. | 0.3 | 0.0 | 0.5 | 0.2 | 0.2 |
| UO-31 | | | | | |
| Renal ca. | 1.9 | 0.4 | 3.1 | 2.0 | 1.9 |
| TK-10 | | | | | |
| Bladder | 4.2 | 0.0 | 5.9 | 5.5 | 5.1 |
| Gastric | 0.9 | 0.0 | 1.7 | 0.9 | 1.2 |
| ca. (liver | | | | | |
| met.) | | | | | |
| NCI-N87 | | | | | |
| Gastric | 0.4 | 0.5 | 0.8 | 0.2 | 0.3 |
| ca. | | | | | |
| KATO | | | | | |
| III | | | | | |
| Colon ca. | 0.0 | 1.5 | 0.2 | 0.2 | 0.2 |
| SW-948 | | | | | |
| Colon ca. | 20.9 | 5.2 | 41.8 | 27.0 | 23.3 |
| SW480 | | | | | |
| Colon ca.* | 13.3 | 4.8 | 16.4 | 12.8 | 10.3 |
| (SW480 | | | | | |
| met) | | | | | |
| SW620 | | | | | |
| Colon ca. | 0.2 | 0.0 | 0.0 | 0.2 | 0.2 |
| HT29 | | | | | |
| Colon ca. | 2.1 | 0.2 | 3.2 | 2.5 | 2.0 |
| HCT-116 | | | | | |
| Colon ca. | 15.0 | 3.6 | 27.0 | 19.1 | 16.7 |
| CaCo-2 | | | | | |
| Colon | 9.0 | 3.3 | 11.0 | 11.9 | 7.6 |
| cancer | | | | | |
| tissue | | | | | |
| Colon ca. | 1.3 | 3.0 | 2.5 | 2.0 | 1.5 |
| SW1116 | | | | | |
| Colon ca. | 0.1 | 0.4 | 0.3 | 0.2 | 0.0 |
| Colo-205 | | | | | |
| Colon ca. | 0.8 | 3.6 | 1.4 | 1.5 | 1.5 |
| SW-48 | | | | | |
| Colon | 20.3 | 5.0 | 28.1 | 23.2 | 18.7 |
| Pool | | | | | |
| Small | 14.0 | 1.7 | 17.1 | 11.2 | 13.0 |
| Intestine | | | | | |
| Pool | | | | | |
| Stomach | 8.1 | 2.3 | 14.3 | 9.5 | 9.3 |
| Pool | | | | | |
| Bone | 6.8 | 1.6 | 14.3 | 10.2 | 8.7 |
| Marrow | | | | | |
| Pool | | | | | |
| Fetal | 10.1 | 2.3 | 25.5 | 24.5 | 21.8 |
| Heart | | | | | |
| Heart | 28.7 | 7.0 | 29.7 | 25.9 | 17.2 |
| Pool | | | | | |
| Lymph | 17.6 | 6.1 | 33.7 | 22.1 | 23.7 |
| Node | | | | | |
| Pool | | | | | |
| Fetal | 31.9 | 5.2 | 54.3 | 48.6 | 46.3 |
| Skeletal | | | | | |
| Muscle | | | | | |
| Skeletal | 17.4 | 9.2 | 29.3 | 29.5 | 25.9 |
| Muscle | | | | | |
| Pool | | | | | |
| Spleen | 0.9 | 0.0 | 1.9 | 2.0 | 1.7 |
| Pool | | | | | |
| Thymus | 4.4 | 2.0 | 10.4 | 8.1 | 9.4 |
| Pool | | | | | |
| CNS | 9.8 | 1.5 | 14.9 | 10.7 | 10.0 |
| cancer | | | | | |
| (glio/astro) | | | | | |
| U87-MG | | | | | |
| CNS | 3.5 | 0.3 | 4.7 | 3.8 | 3.1 |
| cancer | | | | | |
| (glio/astro) | | | | | |
| U-118-MG | | | | | |
| CNS | 1.9 | 0.0 | 2.6 | 2.1 | 1.0 |
| cancer | | | | | |
| (neuro; | | | | | |
| met) | | | | | |
| SK-N-AS | | | | | |
| CNS | 0.1 | 0.0 | 0.0 | 0.1 | 0.2 |
| cancer | | | | | |
| (astro) | | | | | |
| SF-539 | | | | | |
| CNS | 8.1 | 1.1 | 14.9 | 6.5 | 10.0 |
| cancer | | | | | |
| (astro) | | | | | |
| SNB-75 | | | | | |
| CNS | 79.6 | 79.0 | 100.0 | 100.0 | 100.0 |
| cancer | | | | | |
| (glio) | | | | | |
| SNB-19 | | | | | |
| CNS | 8.2 | 0.0 | 11.3 | 8.0 | 7.8 |
| cancer | | | | | |
| (glio) | | | | | |
| SF-295 | | | | | |
| Brain | 3.7 | 0.8 | 7.7 | 6.2 | 4.8 |
| (Amygdala) | | | | | |
| Pool | | | | | |
| Brain | 12.0 | 0.4 | 19.8 | 10.7 | 9.7 |
| (cere- | | | | | |
| bellum) | | | | | |
| Brain | 4.2 | 0.7 | 12.7 | 6.6 | 5.6 |
| (fetal) | | | | | |
| Brain | 7.5 | 3.2 | 11.7 | 8.6 | 6.9 |
| (Hippo- | | | | | |
| campus) | | | | | |
| Pool | | | | | |
| Cerebral | 9.7 | 0.6 | 11.0 | 7.5 | 0.7 |
| Cortex | | | | | |
| Pool | | | | | |
| Brain | 7.4 | 2.2 | 11.7 | 10.4 | 4.7 |
| (Substan- | | | | | |
| tia nigra) | | | | | |
| Pool | | | | | |
| Brain | 7.6 | 2.7 | 13.2 | 9.3 | 0.2 |
| (Thalamus) | | | | | |
| Pool | | | | | |
| Brain | 6.1 | 0.4 | 10.6 | 5.8 | 0.3 |
| (whole) | | | | | |
| Spinal | 10.1 | 2.3 | 14.7 | 11.0 | 7.6 |
| Cord | | | | | |
| Pool | | | | | |
| Adrenal | 3.5 | 0.3 | 9.9 | 3.9 | 3.7 |
| Gland | | | | | |
| Pituitary | 0.9 | 0.0 | 1.1 | 1.2 | 1.1 |
| gland | | | | | |
| Pool | | | | | |
| Salivary | 0.9 | 0.0 | 1.8 | 1.3 | 0.9 |
| Gland | | | | | |
| Thyroid | 2.0 | 0.3 | 3.1 | 2.5 | 2.5 |
| (female) | | | | | |
| Pancreatic | 0.5 | 0.0 | 0.8 | 0.7 | 0.6 |
| ca. | | | | | |
| CAPAN2 | | | | | |
| Pancreas | 1.2 | 0.0 | 2.0 | 1.1 | 1.6 |
| Pool | | | | | |
| | Rel. | Rel. | Rel. | Rel. | Rel. |
| | Exp. (%) | Exp. (%) | Exp. (%) | Exp. (%) | Exp. (%) |
| | Ag6433, | Ag6435, | Ag6440, | Ag6446, | Ag6964, |
| Tissue | Run | Run | Run | Run | Run |
| Name | 277222449 | 277223167 | 277223177 | 277250179 | 278388946 |
| Adipose | 19.5 | 13.2 | 3.7 | 1.7 | 18.8 |
| Melanoma* | 0.3 | 0.9 | 0.0 | 0.1 | 0.7 |
| Hs688(A).T | | | | | |
| Melanoma* | 2.5 | 1.9 | 0.8 | 0.1 | 2.4 |
| Hs688(B).T | | | | | |
| Melanoma* | 0.5 | 0.0 | 0.0 | 0.1 | 0.7 |
| M14 | | | | | |
| Melanoma* | 0.1 | 0.0 | 0.0 | 0.1 | 0.1 |
| LOXIMVI | | | | | |
| Melanoma* | 13.1 | 4.4 | 3.0 | 6.8 | 15.9 |
| SK-MEL-5 | | | | | |
| Squamous | 0.1 | 0.0 | 0.0 | 0.0 | 0.1 |
| cell | | | | | |
| carcinoma | | | | | |
| SCC-4 | | | | | |
| Testis | 10.4 | 10.0 | 3.0 | 5.8 | 9.9 |
| Pool | | | | | |
| Prostate | 1.5 | 1.8 | 1.2 | 7.7 | 4.3 |
| ca.* | | | | | |
| (bone | | | | | |
| met) PC- | | | | | |
| 3 | | | | | |
| Prostate | 15.0 | 10.0 | 2.1 | 1.9 | 10.0 |
| Pool | | | | | |
| Placenta | 0.5 | 0.3 | 0.0 | 0.9 | 0.4 |
| Uterus | 4.7 | 16.2 | 2.3 | 0.3 | 4.1 |
| Pool | | | | | |
| Ovarian | 0.9 | 0.4 | 0.4 | 4.8 | 4.0 |
| ca. | | | | | |
| OVCAR-3 | | | | | |
| Ovarian | 1.2 | 0.9 | 0.5 | 2.5 | 1.7 |
| ca. SK- | | | | | |
| OV-3 | | | | | |
| Ovarian | 0.3 | 0.0 | 0.0 | 0.5 | 0.5 |
| ca. | | | | | |
| OVCAR-4 | | | | | |
| Ovarian | 2.1 | 0.3 | 4.2 | 15.6 | 7.9 |
| ca. | | | | | |
| OVCAR-5 | | | | | |
| Ovarian | 100.0 | 27.0 | 100.0 | 5.4 | 75.8 |
| ca. | | | | | |
| IGROV-1 | | | | | |
| Ovarian | 17.1 | 7.6 | 18.2 | 4.2 | 16.7 |
| ca. | | | | | |
| OVCAR-8 | | | | | |
| Ovary | 4.1 | 4.5 | 0.8 | 0.2 | 2.4 |
| Breast | 0.1 | 0.0 | 0.3 | 0.9 | 0.5 |
| ca. MCF- | | | | | |
| 7 | | | | | |
| Breast | 0.1 | 0.0 | 0.0 | 0.2 | 0.3 |
| ca. | | | | | |
| MDA- | | | | | |
| MB-231 | | | | | |
| Breast | 0.6 | 0.0 | 0.0 | 0.2 | 0.4 |
| ca. BT | | | | | |
| 549 | | | | | |
| Breast | 0.4 | 0.0 | 0.3 | 0.7 | 0.5 |
| ca. T47D | | | | | |
| Breast | 1.0 | 0.7 | 0.3 | 0.0 | 0.8 |
| ca. | | | | | |
| MDA-N | | | | | |
| Breast | 15.5 | 42.9 | 3.5 | 2.0 | 16.7 |
| Pool | | | | | |
| Trachea | 4.6 | 8.3 | 1.4 | 0.5 | 5.6 |
| Lung | 5.2 | 3.9 | 5.3 | 0.5 | 5.1 |
| Fetal | 5.1 | 8.0 | 2.9 | 0.5 | 6.1 |
| Lung | | | | | |
| Lung ca. | 2.3 | 0.2 | 2.0 | 0.4 | 2.3 |
| NCI- | | | | | |
| N417 | | | | | |
| Lung ca. | 4.1 | 0.9 | 6.3 | 100.0 | 44.1 |
| LX-1 | | | | | |
| Lung ca. | 0.3 | 0.0 | 0.0 | 0.1 | 0.1 |
| NCI- | | | | | |
| H146 | | | | | |
| Lung ca. | 6.1 | 0.2 | 0.8 | 0.1 | 3.8 |
| SHP-77 | | | | | |
| Lung ca. | 0.7 | 0.0 | 2.2 | 14.3 | 4.7 |
| A549 | | | | | |
| Lung ca. | 0.6 | 0.0 | 0.3 | 0.0 | 0.5 |
| NCI- | | | | | |
| H526 | | | | | |
| Lung ca. | 3.3 | 0.6 | 2.3 | 15.9 | 10.3 |
| NCI-H23 | | | | | |
| Lung ca. | 0.0 | 0.0 | 0.0 | 0.1 | 0.3 |
| NCI- | | | | | |
| H460 | | | | | |
| Lung ca. | 0.6 | 0.0 | 0.0 | 0.2 | 0.7 |
| HOP-62 | | | | | |
| Lung ca. | 1.4 | 0.0 | 2.5 | 27.7 | 8.9 |
| NCI- | | | | | |
| H522 | | | | | |
| Liver | 0.1 | 0.0 | 0.4 | 5.3 | 2.0 |
| Fetal | 0.2 | 0.3 | 0.8 | 23.0 | 8.2 |
| Liver | | | | | |
| Liver ca. | 0.3 | 0.0 | 0.9 | 7.3 | 2.4 |
| HepG2 | | | | | |
| Kidney | 28.3 | 100.0 | 14.6 | 5.3 | 32.8 |
| Pool | | | | | |
| Fetal | 4.1 | 12.1 | 3.4 | 20.2 | 11.5 |
| Kidney | | | | | |
| Renal ca. | 0.5 | 0.0 | 0.0 | 1.7 | 0.9 |
| 786-0 | | | | | |
| Renal ca. | 0.0 | 0.0 | 3.8 | 23.0 | 8.5 |
| A498 | | | | | |
| Renal ca. | 2.1 | 0.0 | 0.5 | 3.8 | 2.5 |
| ACHN | | | | | |
| Renal ca. | 0.4 | 0.0 | 0.0 | 0.7 | 0.3 |
| UO-31 | | | | | |
| Renal ca. | 2.9 | 0.7 | 0.5 | 6.4 | 4.6 |
| TK-10 | | | | | |
| Bladder | 4.2 | 6.6 | 0.9 | 3.2 | 6.7 |
| Gastric | 1.3 | 0.0 | 0.8 | 17.8 | 6.7 |
| ca. (liver | | | | | |
| met.) | | | | | |
| NCI-N87 | | | | | |
| Gastric | 0.2 | 0.3 | 0.4 | 1.3 | 0.9 |
| ca. | | | | | |
| KATO | | | | | |
| III | | | | | |
| Colon ca. | 0.0 | 0.0 | 2.2 | 6.1 | 1.2 |
| SW-948 | | | | | |
| Colon ca. | 26.8 | 4.4 | 6.3 | 39.0 | 33.7 |
| SW480 | | | | | |
| Colon ca.* | 13.0 | 1.7 | 7.2 | 71.2 | 25.0 |
| (SW480 | | | | | |
| met) | | | | | |
| SW620 | | | | | |
| Colon ca. | 0.1 | 0.0 | 0.3 | 3.5 | 0.3 |
| HT29 | | | | | |
| Colon ca. | 2.3 | 0.5 | 0.6 | 6.4 | 4.3 |
| HCT-116 | | | | | |
| Colon ca. | 16.5 | 7.6 | 6.5 | 78.5 | 38.2 |
| CaCo-2 | | | | | |
| Colon | 10.0 | 5.6 | 4.4 | 21.9 | 20.4 |
| cancer | | | | | |
| tissue | | | | | |
| Colon ca. | 1.1 | 1.1 | 2.1 | 19.5 | 6.0 |
| SW1116 | | | | | |
| Colon ca. | 0.0 | 0.0 | 1.3 | 3.0 | 0.8 |
| Colo-205 | | | | | |
| Colon ca. | 1.0 | 0.0 | 3.0 | 4.2 | 2.6 |
| SW-48 | | | | | |
| Colon | 18.7 | 44.8 | 8.1 | 3.1 | 20.6 |
| Pool | | | | | |
| Small | 11.4 | 26.8 | 2.0 | 2.5 | 10.4 |
| Intestine | | | | | |
| Pool | | | | | |
| Stomach | 9.5 | 24.0 | 4.2 | 1.1 | 10.7 |
| Pool | | | | | |
| Bone | 16.6 | 25.9 | 3.5 | 1.1 | 12.5 |
| Marrow | | | | | |
| Pool | | | | | |
| Fetal | 21.6 | 31.6 | 8.6 | 2.7 | 20.7 |
| Heart | | | | | |
| Heart | 29.7 | 23.5 | 10.7 | 3.4 | 26.1 |
| Pool | | | | | |
| Lymph | 23.0 | 64.6 | 6.7 | 2.8 | 24.7 |
| Node | | | | | |
| Pool | | | | | |
| Fetal | 47.0 | 46.7 | 19.2 | 57.0 | 50.7 |
| Skeletal | | | | | |
| Muscle | | | | | |
| Skeletal | 34.2 | 24.7 | 22.7 | 24.3 | 32.3 |
| Muscle | | | | | |
| Pool | | | | | |
| Spleen | 1.4 | 2.4 | 0.6 | 2.6 | 3.1 |
| Pool | | | | | |
| Thymus | 8.8 | 18.4 | 3.1 | 1.4 | 7.0 |
| Pool | | | | | |
| CNS | 13.2 | 5.8 | 2.2 | 6.3 | 14.1 |
| cancer | | | | | |
| (glio/astro) | | | | | |
| U87-MG | | | | | |
| CNS | 3.8 | 1.5 | 0.8 | 5.1 | 5.8 |
| cancer | | | | | |
| (glio/astro) | | | | | |
| U-118-MG | | | | | |
| CNS | 2.0 | 0.7 | 0.5 | 3.9 | 2.6 |
| cancer | | | | | |
| (neuro; | | | | | |
| met) | | | | | |
| SK-N-AS | | | | | |
| CNS | 0.2 | 0.2 | 0.2 | 0.3 | 0.1 |
| cancer | | | | | |
| (astro) | | | | | |
| SF-539 | | | | | |
| CNS | 11.4 | 3.1 | 2.8 | 2.4 | 9.7 |
| cancer | | | | | |
| (astro) | | | | | |
| SNB-75 | | | | | |
| CNS | 98.6 | 12.8 | 97.9 | 5.2 | 100.0 |
| cancer | | | | | |
| (glio) | | | | | |
| SNB-19 | | | | | |
| CNS | 5.9 | 0.0 | 1.5 | 14.9 | 14.8 |
| cancer | | | | | |
| (glio) | | | | | |
| SF-295 | | | | | |
| Brain | 6.0 | 7.9 | 4.4 | 1.1 | 5.3 |
| (Amygdala) | | | | | |
| Pool | | | | | |
| Brain | 11.5 | 1.8 | 1.2 | 1.4 | 9.7 |
| (cere- | | | | | |
| bellum) | | | | | |
| Brain | 6.2 | 8.4 | 2.1 | 1.1 | 6.4 |
| (fetal) | | | | | |
| Brain | 8.3 | 9.9 | 4.3 | 2.0 | 10.2 |
| (Hippo- | | | | | |
| campus) | | | | | |
| Pool | | | | | |
| Cerebral | 7.0 | 1.8 | 2.0 | 2.0 | 8.7 |
| Cortex | | | | | |
| Pool | | | | | |
| Brain | 10.1 | 4.2 | 2.0 | 1.1 | 9.3 |
| (Substan- | | | | | |
| tia nigra) | | | | | |
| Pool | | | | | |
| Brain | 8.5 | 9.1 | 2.8 | 3.2 | 8.7 |
| (Thalamus) | | | | | |
| Pool | | | | | |
| Brain | 4.9 | 3.3 | 1.9 | 1.9 | 8.7 |
| (whole) | | | | | |
| Spinal | 12.1 | 13.1 | 4.2 | 2.9 | 9.0 |
| Cord | | | | | |
| Pool | | | | | |
| Adrenal | 6.4 | 7.4 | 0.9 | 0.7 | 4.1 |
| Gland | | | | | |
| Pituitary | 0.8 | 1.8 | 0.6 | 0.4 | 0.5 |
| gland | | | | | |
| Pool | | | | | |
| Salivary | 1.3 | 2.3 | 0.0 | 0.2 | 1.0 |
| Gland | | | | | |
| Thyroid | 3.5 | 3.3 | 1.3 | 0.8 | 2.3 |
| (female) | | | | | |
| Pancreatic | 1.0 | 0.5 | 0.6 | 4.6 | 2.2 |
| ca. | | | | | |
| CAPAN2 | | | | | |
| Pancreas | 1.7 | 3.5 | 1.0 | 2.6 | 2.3 |
| Pool | | | | | |

TABLE ARQ
Panel 4.1D

| | Rel. | Rel. | Rel. | Rel. | Rel. | Rel. | Rel. | Rel. | Rel. |
|---|---|---|---|---|---|---|---|---|---|
| | Exp. (%) | Exp. (%) | Exp. (%) | Exp. (%) | Exp. (%) | Exp. (%) | Exp. (%) | Exp. (%) | Exp. (%) |
| | Ag4983, | Ag6413, | Ag6425, | Ag6428, | Ag6431, | Ag6433, | Ag6435, | Ag6446, | Ag6447, |
| Tissue | Run | Run | Run | Run | Run | Run | Run | Run | Run |
| Name | 218623570 | 269239947 | 268713999 | 268767535 | 268767577 | 268713319 | 268713480 | 268761804 | 268761806 |
| Secondary | 0.1 | 0.3 | 0.0 | 1.3 | 0.7 | 0.6 | 0.0 | 0.0 | 0.0 |
| Th1 act | | | | | | | | | |
| Secondary | 0.5 | 0.3 | 0.0 | 1.2 | 0.8 | 0.6 | 0.0 | 0.0 | 0.0 |
| Th2 act | | | | | | | | | |
| Secondary | 0.0 | 0.0 | 0.0 | 0.0 | 0.7 | 0.5 | 0.0 | 0.0 | 0.0 |
| Tr1 act | | | | | | | | | |
| Secondary | 0.1 | 0.0 | 0.0 | 0.0 | 0.0 | 0.0 | 0.0 | 0.0 | 0.0 |
| Th1 rest | | | | | | | | | |
| Secondary | 0.3 | 0.0 | 0.0 | 0.0 | 0.0 | 0.0 | 0.7 | 0.0 | 0.0 |
| Th2 rest | | | | | | | | | |
| Secondary | 0.1 | 0.3 | 0.0 | 0.4 | 0.0 | 0.0 | 0.0 | 0.0 | 0.0 |
| Tr1 rest | | | | | | | | | |
| Primary | 0.0 | 0.0 | 0.0 | 0.0 | 0.0 | 0.0 | 0.0 | 0.0 | 0.0 |
| Th1 act | | | | | | | | | |
| Primary | 0.2 | 0.4 | 0.0 | 0.3 | 0.4 | 0.0 | 0.7 | 0.0 | 0.0 |
| Th2 act | | | | | | | | | |
| Primary Tr1 | 0.1 | 0.0 | 0.0 | 0.7 | 0.7 | 0.0 | 0.0 | 0.0 | 0.0 |
| act | | | | | | | | | |
| Primary | 0.0 | 0.0 | 0.0 | 0.1 | 0.3 | 0.0 | 0.0 | 0.4 | 0.0 |
| Th1 rest | | | | | | | | | |
| Primary | 0.0 | 0.0 | 0.0 | 0.4 | 0.2 | 0.0 | 0.0 | 0.0 | 0.0 |
| Th2 rest | | | | | | | | | |
| Primary Tr1 | 0.3 | 0.0 | 0.0 | 0.0 | 0.0 | 0.7 | 0.0 | 0.0 | 0.0 |
| rest | | | | | | | | | |
| CD45RA | 0.4 | 2.8 | 0.0 | 5.4 | 2.4 | 0.3 | 0.8 | 3.7 | 0.0 |
| CD4 | | | | | | | | | |
| lymphocyte | | | | | | | | | |
| act | | | | | | | | | |
| CD45RO | 0.1 | 2.2 | 0.0 | 1.5 | 0.7 | 0.8 | 1.6 | 0.0 | 0.0 |
| CD4 | | | | | | | | | |
| lymphocyte | | | | | | | | | |
| act | | | | | | | | | |
| CD8 | 0.4 | 0.9 | 0.0 | 0.7 | 0.0 | 0.0 | 0.0 | 0.0 | 0.0 |
| lymphocyte | | | | | | | | | |
| act | | | | | | | | | |
| Secondary | 0.1 | 0.0 | 0.0 | 8.8 | 0.0 | 0.0 | 0.0 | 0.0 | 0.0 |
| CD8 | | | | | | | | | |
| lymphocyte | | | | | | | | | |
| rest | | | | | | | | | |
| Secondary | 0.0 | 0.1 | 0.0 | 0.4 | 0.3 | 0.0 | 0.0 | 0.0 | 0.0 |
| CD8 | | | | | | | | | |
| lymphocyte | | | | | | | | | |
| act | | | | | | | | | |
| CD4 | 0.1 | 0.0 | 0.0 | 0.5 | 0.4 | 0.0 | 0.0 | 0.0 | 0.0 |
| lymphocyte | | | | | | | | | |
| none | | | | | | | | | |
| 2ry | 0.3 | 0.2 | 0.0 | 0.0 | 0.0 | 0.0 | 0.0 | 0.0 | 0.0 |
| Th1/Th2/ | | | | | | | | | |
| Tr1_anti- | | | | | | | | | |
| CD95 | | | | | | | | | |
| CH11 | | | | | | | | | |
| LAK cells | 5.6 | 5.0 | 2.7 | 11.8 | 3.8 | 6.4 | 6.1 | 19.5 | 0.0 |
| rest | | | | | | | | | |
| LAK cells | 0.4 | 0.3 | 0.0 | 0.0 | 0.0 | 0.0 | 0.0 | 1.1 | 0.0 |
| IL-2 | | | | | | | | | |
| LAK cells | 0.2 | 0.0 | 0.0 | 0.0 | 0.0 | 0.0 | 0.0 | 0.0 | 0.0 |
| IL-2 + | | | | | | | | | |
| IL-12 | | | | | | | | | |
| LAK cells | 0.1 | 0.3 | 0.0 | 0.0 | 0.0 | 0.0 | 0.0 | 0.0 | 0.0 |
| IL-2 + | | | | | | | | | |
| IFN gamma | | | | | | | | | |
| LAK cells | 0.0 | 0.0 | 0.0 | 0.0 | 0.0 | 0.0 | 0.0 | 0.0 | 0.0 |
| IL-2 + | | | | | | | | | |
| IL-18 | | | | | | | | | |
| LAK cells | 4.5 | 4.0 | 15.7 | 15.1 | 6.3 | 4.3 | 6.1 | 41.5 | 0.0 |
| PMA/ | | | | | | | | | |
| ionomycin | | | | | | | | | |
| NK Cells | 0.9 | 0.1 | 0.0 | 3.4 | 2.5 | 0.0 | 0.0 | 0.0 | 0.0 |
| IL-2 rest | | | | | | | | | |
| Two Way | 1.4 | 1.1 | 0.0 | 2.2 | 1.3 | 0.7 | 0.9 | 2.1 | 0.0 |
| MLR 3 day | | | | | | | | | |
| Two Way | 4.5 | 0.9 | 0.0 | 0.8 | 0.9 | 0.9 | 0.0 | 5.8 | 0.0 |
| MLR 5 day | | | | | | | | | |
| Two Way | 2.3 | 0.7 | 13.2 | 1.1 | 2.6 | 2.6 | 2.9 | 5.8 | 0.0 |
| MLR 7 day | | | | | | | | | |
| PBMC rest | 0.1 | 0.0 | 0.0 | 0.0 | 0.0 | 0.0 | 0.0 | 0.0 | 0.0 |
| PBMC | 0.6 | 0.0 | 0.0 | 1.3 | 0.0 | 0.3 | 0.0 | 0.0 | 0.0 |
| PWM | | | | | | | | | |
| PBMC | 0.3 | 0.2 | 0.0 | 0.6 | 0.7 | 0.0 | 0.0 | 0.0 | 0.0 |
| PHA-L | | | | | | | | | |
| Ramos (B | 0.1 | 0.0 | 0.0 | 0.0 | 0.0 | 0.0 | 0.0 | 0.0 | 0.0 |
| cell) none | | | | | | | | | |
| Ramos (B | 0.0 | 0.0 | 0.0 | 0.7 | 0.2 | 0.0 | 0.0 | 0.0 | 0.0 |
| cell) | | | | | | | | | |
| ionomycin | | | | | | | | | |
| B | 0.5 | 0.0 | 0.0 | 0.0 | 0.0 | 0.0 | 0.0 | 0.0 | 0.0 |
| lymphocytes | | | | | | | | | |
| PWM | | | | | | | | | |
| B | 0.2 | 0.0 | 0.0 | 0.9 | 0.0 | 0.6 | 0.0 | 0.0 | 0.0 |
| lymphocytes | | | | | | | | | |
| CD40L | | | | | | | | | |
| and IL-4 | | | | | | | | | |
| EOL-1 | 3.7 | 2.6 | 9.1 | 29.1 | 8.1 | 10.2 | 0.0 | 3.6 | 0.0 |
| dbcAMP | | | | | | | | | |
| EOL-1 | 1.6 | 0.7 | 0.0 | 0.0 | 2.7 | 1.6 | 1.0 | 0.0 | 0.0 |
| dbcAMP | | | | | | | | | |
| PMA/ | | | | | | | | | |
| ionomycin | | | | | | | | | |
| Dendritic | 5.6 | 3.1 | 13.8 | 4.1 | 5.3 | 4.2 | 0.7 | 100.0 | 0.0 |
| cells none | | | | | | | | | |
| Dendritic | 1.6 | 0.3 | 0.0 | 1.0 | 0.7 | 1.6 | 0.0 | 2.4 | 0.0 |
| cells LPS | | | | | | | | | |
| Dendritic | 2.0 | 1.6 | 3.3 | 0.5 | 0.2 | 0.3 | 1.6 | 4.3 | 0.0 |
| cells anti- | | | | | | | | | |
| CD40 | | | | | | | | | |
| Monocytes | 0.2 | 0.0 | 0.0 | 0.4 | 0.0 | 0.0 | 0.0 | 0.0 | 0.0 |
| rest | | | | | | | | | |
| Monocytes | 2.2 | 3.3 | 0.0 | 5.7 | 1.8 | 1.0 | 0.0 | 1.6 | 0.4 |
| LPS | | | | | | | | | |
| Macrophages | 0.9 | 1.8 | 0.0 | 0.6 | 0.6 | 1.4 | 0.0 | 6.9 | 0.0 |
| rest | | | | | | | | | |
| Macrophages | 7.5 | 4.0 | 0.0 | 5.4 | 6.3 | 2.1 | 0.8 | 6.5 | 0.0 |
| LPS | | | | | | | | | |
| HUVEC | 0.1 | 0.0 | 0.0 | 0.0 | 0.0 | 0.0 | 0.0 | 0.0 | 0.0 |
| none | | | | | | | | | |
| HUVEC | 0.0 | 0.0 | 0.0 | 0.0 | 0.3 | 0.0 | 0.0 | 0.0 | 0.0 |
| starved | | | | | | | | | |
| HUVEC | 0.0 | 0.0 | 0.0 | 0.0 | 0.5 | 0.0 | 0.0 | 0.0 | 0.0 |
| IL-1beta | | | | | | | | | |
| HUVEC | 0.2 | 0.0 | 0.0 | 0.0 | 0.0 | 0.0 | 0.0 | 0.0 | 0.0 |
| IFN gamma | | | | | | | | | |
| HUVEC TNF | 0.0 | 0.0 | 0.0 | 0.0 | 0.0 | 0.0 | 0.6 | 0.0 | 0.0 |
| alpha + | | | | | | | | | |
| IFN gamma | | | | | | | | | |
| HUVEC TNF | 0.6 | 0.0 | 0.0 | 0.0 | 0.4 | 0.0 | 0.0 | 0.0 | 0.0 |
| alpha + | | | | | | | | | |
| IL4 | | | | | | | | | |
| HUVEC | 0.0 | 0.0 | 0.0 | 0.4 | 0.3 | 0.0 | 0.0 | 0.0 | 0.0 |
| IL-11 | | | | | | | | | |
| Lung | 0.2 | 0.3 | 0.0 | 0.4 | 0.0 | 0.0 | 0.0 | 0.0 | 0.0 |
| Microvas- | | | | | | | | | |
| cular | | | | | | | | | |
| EC none | | | | | | | | | |
| Lung | 0.1 | 0.0 | 0.0 | 0.0 | 0.0 | 0.0 | 0.0 | 0.0 | 0.0 |
| Microvas- | | | | | | | | | |
| cular EC | | | | | | | | | |
| TNFalpha + | | | | | | | | | |
| IL-1beta | | | | | | | | | |
| Micro- | 0.1 | 0.0 | 0.0 | 0.0 | 0.0 | 0.0 | 0.0 | 0.0 | 0.0 |
| vascular | | | | | | | | | |
| Dermal | | | | | | | | | |
| EC none | | | | | | | | | |
| Micros- | 0.1 | 0.0 | 0.0 | 0.0 | 0.0 | 0.0 | 0.0 | 0.0 | 0.0 |
| vasular | | | | | | | | | |
| Dermal EC | | | | | | | | | |
| TNFalpha + | | | | | | | | | |
| IL-1beta | | | | | | | | | |
| Bronchial | 0.0 | 0.0 | 0.0 | 0.0 | 0.0 | 0.0 | 0.0 | 0.0 | 0.0 |
| epithelium | | | | | | | | | |
| TNFalpha + | | | | | | | | | |
| IL1beta | | | | | | | | | |
| Small | 0.0 | 0.0 | 0.0 | 0.0 | 0.0 | 0.0 | 0.0 | 0.0 | 0.0 |
| airway | | | | | | | | | |
| epithelium | | | | | | | | | |
| none | | | | | | | | | |
| Small | 0.3 | 0.0 | 0.0 | 0.0 | 0.0 | 0.0 | 0.0 | 0.0 | 0.0 |
| airway | | | | | | | | | |
| epithelium | | | | | | | | | |
| TNFalpha + | | | | | | | | | |
| IL-1beta | | | | | | | | | |
| Coronery | 0.1 | 0.6 | 0.0 | 0.0 | 0.0 | 0.0 | 0.5 | 0.0 | 0.3 |
| artery SMC | | | | | | | | | |
| rest | | | | | | | | | |
| Coronery | 0.4 | 0.9 | 6.2 | 0.3 | 1.5 | 0.5 | 0.0 | 0.0 | 0.0 |
| artery SMC | | | | | | | | | |
| TNFalpha + | | | | | | | | | |
| IL-1beta | | | | | | | | | |
| Astrocytes | 67.8 | 97.3 | 100.0 | 100.0 | 100.0 | 100.0 | 100.0 | 1.0 | 54.3 |
| rest | | | | | | | | | |
| Astrocytes | 100.0 | 100.0 | 74.2 | 97.3 | 74.7 | 65.1 | 97.9 | 1.4 | 100.0 |
| TNFalpha + | | | | | | | | | |
| IL-1beta | | | | | | | | | |
| KU-812 | 0.1 | 0.0 | 0.0 | 0.0 | 0.4 | 0.0 | 0.0 | 0.4 | 0.0 |
| (Basophil) | | | | | | | | | |
| rest | | | | | | | | | |
| KU-812 | 0.0 | 0.0 | 0.0 | 0.0 | 0.0 | 0.0 | 0.0 | 0.0 | 0.0 |
| (Basophil) | | | | | | | | | |
| PMA/ | | | | | | | | | |
| ionomycin | | | | | | | | | |
| CCD1106 | 0.2 | 0.0 | 0.0 | 0.0 | 0.8 | 0.0 | 0.0 | 0.0 | 0.0 |
| (Keratino- | | | | | | | | | |
| cytes) | | | | | | | | | |
| none | | | | | | | | | |
| CCD1106 | 0.3 | 0.0 | 0.0 | 0.0 | 0.0 | 0.0 | 0.0 | 0.0 | 0.0 |
| (Keratino- | | | | | | | | | |
| cytes) | | | | | | | | | |
| TNFalpha + | | | | | | | | | |
| IL-1beta | | | | | | | | | |
| Liver | 2.3 | 7.2 | 4.6 | 2.6 | 6.7 | 2.2 | 5.1 | 15.3 | 0.6 |
| cirrhosis | | | | | | | | | |
| NCI-H292 | 0.3 | 0.3 | 0.0 | 1.7 | 0.6 | 0.0 | 0.0 | 0.0 | 0.0 |
| none | | | | | | | | | |
| NCI-H292 | 0.3 | 0.0 | 0.0 | 0.0 | 0.5 | 0.0 | 0.0 | 0.0 | 0.0 |
| IL-4 | | | | | | | | | |
| NCI-H292 | 0.3 | 0.0 | 0.0 | 0.7 | 0.5 | 0.0 | 0.0 | 1.4 | 0.0 |
| IL-9 | | | | | | | | | |
| NCI-H292 | 0.6 | 0.6 | 0.0 | 0.9 | 0.9 | 0.0 | 0.0 | 0.9 | 0.0 |
| IL-13 | | | | | | | | | |
| NCI-H292 | 0.2 | 0.0 | 0.0 | 0.5 | 0.6 | 0.6 | 0.0 | 0.0 | 0.0 |
| IFN gamma | | | | | | | | | |
| HPAEC | 0.0 | 0.3 | 0.0 | 0.0 | 0.0 | 0.0 | 0.0 | 0.0 | 0.0 |
| none | | | | | | | | | |
| HPAEC | 0.0 | 0.3 | 0.0 | 0.0 | 0.0 | 1.1 | 0.0 | 0.0 | 0.0 |
| TNF | | | | | | | | | |
| alpha + | | | | | | | | | |
| IL-1 beta | | | | | | | | | |
| Lung | 29.7 | 62.9 | 31.4 | 95.9 | 65.5 | 28.5 | 62.9 | 3.1 | 26.2 |
| fibroblast | | | | | | | | | |
| none | | | | | | | | | |
| Lung | 16.0 | 36.9 | 22.2 | 48.6 | 39.8 | 19.3 | 25.2 | 0.4 | 28.3 |
| fibroblast | | | | | | | | | |
| TNF | | | | | | | | | |
| alpha + | | | | | | | | | |
| IL-1 beta | | | | | | | | | |
| Lung | 26.1 | 28.7 | 19.1 | 27.4 | 21.2 | 25.9 | 23.3 | 0.9 | 16.0 |
| fibroblast | | | | | | | | | |
| IL-4 | | | | | | | | | |
| Lung | 28.5 | 42.0 | 23.5 | 24.0 | 26.8 | 25.9 | 20.4 | 2.0 | 9.3 |
| fibroblast | | | | | | | | | |
| IL-9 | | | | | | | | | |
| Lung | 31.6 | 14.6 | 4.5 | 11.9 | 10.4 | 16.0 | 15.0 | 1.3 | 4.3 |
| fibroblast | | | | | | | | | |
| IL-13 | | | | | | | | | |
| Lung | 20.4 | 32.8 | 15.7 | 55.9 | 46.3 | 25.0 | 29.9 | 1.0 | 25.2 |
| fibroblast | | | | | | | | | |
| IFN gamma | | | | | | | | | |
| Dermal | 2.5 | 2.9 | 0.0 | 6.0 | 6.3 | 2.3 | 5.6 | 1.1 | 0.0 |
| fibroblast | | | | | | | | | |
| CCD1070 | | | | | | | | | |
| rest | | | | | | | | | |
| Dermal | 1.1 | 1.3 | 0.0 | 2.7 | 0.8 | 5.1 | 0.8 | 0.0 | 1.1 |
| fibroblast | | | | | | | | | |
| CCD1070 | | | | | | | | | |
| TNF alpha | | | | | | | | | |
| Dermal | 1.9 | 2.9 | 0.0 | 5.6 | 1.3 | 1.4 | 0.7 | 0.0 | 1.6 |
| fibroblast | | | | | | | | | |
| CCD1070 | | | | | | | | | |
| IL-1 beta | | | | | | | | | |
| Dermal | 9.3 | 20.3 | 8.5 | 30.6 | 20.2 | 13.6 | 20.0 | 0.0 | 4.9 |
| fibroblast | | | | | | | | | |
| IFN gamma | | | | | | | | | |
| Dermal | 10.7 | 14.6 | 4.1 | 30.8 | 19.8 | 13.9 | 22.7 | 1.4 | 13.5 |
| fibroblast | | | | | | | | | |
| IL-4 | | | | | | | | | |
| Dermal | 24.8 | 42.3 | 8.0 | 54.3 | 46.7 | 19.8 | 20.7 | 1.6 | 15.8 |
| Fibroblasts | | | | | | | | | |
| rest | | | | | | | | | |
| Neutrophils | 0.7 | 0.0 | 0.0 | 0.9 | 0.4 | 0.0 | 1.2 | 0.0 | 0.0 |
| TNFa + LPS | | | | | | | | | |
| Neutrophils | 0.1 | 0.0 | 0.0 | 0.0 | 0.3 | 0.0 | 0.0 | 0.0 | 0.0 |
| rest | | | | | | | | | |
| Colon | 7.9 | 4.7 | 4.0 | 4.6 | 9.5 | 7.0 | 7.9 | 1.8 | 4.8 |
| Lung | 2.2 | 1.2 | 0.0 | 2.8 | 4.6 | 1.3 | 1.6 | 0.8 | 0.0 |
| Thymus | 3.1 | 0.8 | 0.0 | 0.0 | 0.4 | 0.0 | 2.0 | 0.0 | 0.0 |
| Kidney | 4.2 | 4.4 | 4.9 | 7.8 | 9.7 | 5.3 | 10.2 | 50.0 | 0.6 |

TABLE ARR
general oncology screening panel_v_2.4

| | Rel. Exp. (%) | Rel. Exp. (%) |
|---|---|---|
| | Ag4983, Run | Ag6442, Run |
| Tissue Name | 260281959 | 264979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell carcinoma 3 | 5.6 | 8.3 |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate adenocarcinoma 1 | 9.2 | 7.5 |
| Prostate adenocarcinoma 2 | 3.5 | 8.0 |
| Prostate adenocarcinoma 3 | 14.3 | 9.0 |
| Prostate adenocarcinoma 4 | 16.4 | 9.1 |
| Prostate NAT 5 | 16.8 | 9.9 |
| Prostate adenocarcinoma 6 | 3.2 | 7.7 |
| Prostate adenocarcinoma 7 | 9.2 | 17.3 |
| Prostate adenocarcinoma 8 | 3.0 | 0.0 |
| Prostate adenocarcinoma 9 | 27.0 | 33.9 |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0

Summary: Ag4983/Ag6431/Ag6428/Ag6431/Ag6435/Ag6440/Ag6442/Ag6446/Ag6447 This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

General_screening_panel_v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag6442 Highest expression of this gene is seen in skeletal muscle (CT-28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers.

This expression pattern is consistent with the expression seen in panel 1.4. See panel 1.4 for further discussion on the utility of these genes.

General_screening_panel_v1.6

Summary: Ag6413/Ag6425/Ag6428/Ag6430/Ag6431/Ag6440/Ag6442/Ag6446/Ag6964 Eight experiments with seven different probe and primer sets are in very good agreement. Highest expression of this gene is detected in kidney, ovarian cancer IGROV-1 cell line, lung cancer LX-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Panel 4.1D

Summary: Ag4983/Ag6413/Ag6428/Ag6430/Ag6431/Ag6433/Ag6439/Ag6442 Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AS. CG56054-12: Integrin Alpha 7-Like Protein.

Expression of gene CG56054-12 was assessed using the primer-probe sets Ag4983, Ag6442, Ag6424, Ag6425, Ag6428, Ag6430, Ag6431, Ag6439, Ag6413 and Ag6964, described in Tables ASA, ASB, ASC, ASD, ASE, ASF, ASG, ASH, ASI and ASJ. Results of the RTQ-PCR runs are shown in Tables ASK, ASL, ASM, ASN, ASO and ASP.

TABLE ASA

Probe Name Ag4983

| | | | | |
|---|---|---|---|---|
| | | | Start | |
| Primers | Sequences | Length | Position | SEQ ID No |

| | | | | |
|---|---|---|---|---|
| Forward | 5′-ccaggtcaccttctacctcatc-3′ | 22 | 2435 | 570 |
| Probe | TET-5′- | 24 | 2457 | 571 |
| | cttagcacctccgggatcagcatt-3′-TAMRA | | | |
| Reverse | 5′-aacagcagctctacctccagtt-3′ | 22 | 2491 | 572 |

TABLE ASB

Probe Name Ag6442

| | | | | |
|---|---|---|---|---|
| | | | Start | |
| Primers | Sequences | Length | Position | SEQ ID No |

| | | | | |
|---|---|---|---|---|
| Forward | 5′-gatgtggacagtagggatagga-3′ | 22 | 2874 | 573 |
| Probe | TET-5′- | 21 | 2913 | 574 |
| | ccacctgagcagcaggagcct-3′-TAMRA | | | |
| Reverse | 5′-gcgcagtccagggtg-3′ | 15 | 2999 | 575 |

TABLE ASC

Probe Name Ag6424

| | | | | |
|---|---|---|---|---|
| | | | Start | |
| Primers | Sequences | Length | Position | SEQ ID No |

| | | | | |
|---|---|---|---|---|
| Forward | 5′-ttgggttctgccagca-3′ | 16 | 742 | 576 |
| Probe | TET-5′- | 20 | 761 | 577 |
| | cacagctgccgccttctccc-3′-TAMRA | | | |
| Reverse | 5′-aaaagcaaccccttccaa-3′ | 18 | 824 | 578 |

TABLE ASD

Probe Name Ag6425

| | | | | |
|---|---|---|---|---|
| | | | | SEQ |
| | | | Start | ID |
| Primers | Sequences | Length | Position | No |

| | | | | |
|---|---|---|---|---|
| Forward | 5′-cggatgcacaccccat-3′ | 16 | 3389 | 579 |
| Probe | TET-5′- | 18 | 3421 | 580 |
| | catcccgagctgggcccc-3′- | | | |
| | TAMRA | | | |
| Reverse | 5′-gccctggatgcccat-3′ | 15 | 3440 | 581 |

TABLE ASE

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1394 | 582 |
| Probe | TET-5′-ccttcacaggtggagggc-3′-TAMRA | 21 | 1434 | 583 |
| Reverse | 5′-agggagtagccgaagctct-3′ | 19 | 1471 | 584 |

TABLE ASE

Probe Name Ag6430

| | | | Start | |
|---|---|---|---|---|
| Primers | Sequences | Length | Position | SEQ ID No |
| Forward | 5′-gtgaccaacattgatagctcaga-3′ | 23 | 843 | 585 |
| Probe | TET-5′-ccccgaccagctggtgtataaaactttg-3′-TAMRA | 28 | 866 | 586 |
| Reverse | 5′-ggagccggtcagca-3′ | 15 | 899 | 587 |

TABLE ASG

Probe Name Ag6431

| | | | Start | |
|---|---|---|---|---|
| Primers | Sequences | Length | Position | SEQ ID No |
| Forward | 5′-aaacatcaccctggactgc-3′ | 19 | 2993 | 588 |
| Probe | TET-5′-tggtgttcagctgcccactctacag-3′-TAMRA | 25 | 3034 | 589 |
| Reverse | 5′-ccgcgcggtcaaa-3′ | 13 | 3060 | 590 |

TABLE ASH

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 3250 | 591 |
| Probe | TET-5′-ccctggtgggtcatcctcctg-3′-TAMRA | 21 | 3270 | 592 |
| Reverse | 5′-gaagaatcccatcttccacag-3′ | 21 | 3336 | 593 |

TABLE ASI

Probe Name Ag6413

| | | | | |
|---|---|---|---|---|
| Primers | Sequences | Length | Start Position | SEQ ID No |

| | | | | |
|---|---|---|---|---|
| Forward | 5′-ggtgaagacaagatctgccag-3′ | 21 | 2073 | 594 |
| Probe | TET-5′-tgtacccgggtcagcgacacg-3′-TAMRA | 21 | 2124 | 595 |
| Reverse | 5′-gtgttgttccatccacatc-3′ | 20 | 2166 | 596 |

TABLE ASJ

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatgca-3′ | 15 | 3079 | 597 |
| Probe | TET-5′-actctacagctttgaccgcgcgg-3′-TAMRA | 23 | 3050 | 598 |
| Reverse | 5′-gccaactgtgtggtggttca-3′ | 19 | 3024 | 599 |

TABLE ASK
CNS_neurodegeneration_v1.0

| | Rel. | Rel. | Rel. | Rel. | Rel. | Rel. | Rel. |
|---|---|---|---|---|---|---|---|
| | Exp. (%) | Exp. (%) | Exp. (%) | Exp. (%) | Exp. (%) | Exp. (%) | Exp. (%) |
| | Ag4983, | Ag6413, | Ag6428, | Ag6430, | Ag6431, | Ag6439, | Ag6442, |
| Tissue | Run | Run | Run | Run | Run | Run | Run |
| Name | 218649223 | 269253983 | 266937081 | 266937085 | 268030722 | 269254002 | 264979298 |
| AD 1 Hippo | 23.7 | 24.8 | 18.0 | 20.0 | 18.8 | 21.6 | 19.2 |
| AD 2 Hippo | 41.2 | 52.9 | 32.3 | 48.0 | 28.7 | 28.9 | 49.7 |
| AD 3 Hippo | 8.9 | 6.4 | 3.7 | 11.6 | 7.5 | 6.1 | 20.4 |
| AD 4 Hippo | 14.8 | 25.5 | 10.7 | 17.1 | 18.8 | 17.6 | 5.6 |
| AD 5 Hippo | 44.8 | 41.8 | 53.2 | 39.2 | 38.4 | 42.6 | 57.4 |
| AD 6 Hippo | 100.0 | 100.0 | 100.0 | 100.0 | 100.0 | 100.0 | 90.1 |
| Control 2 | 24.3 | 36.1 | 18.7 | 17.9 | 29.5 | 32.5 | 28.5 |
| Hippo | | | | | | | |
| Control 4 | 42.9 | 43.8 | 27.0 | 38.4 | 32.3 | 37.9 | 86.5 |
| Hippo | | | | | | | |
| Control | 14.2 | 11.4 | 4.6 | 10.2 | 6.0 | 6.4 | 0.0 |
| (Path) 3 | | | | | | | |
| Hippo | | | | | | | |
| AD 1 | 23.3 | 15.9 | 12.9 | 12.1 | 17.1 | 24.5 | 16.8 |
| Temporal | | | | | | | |
| Ctx | | | | | | | |
| AD 2 | 41.5 | 47.3 | 31.0 | 36.6 | 39.8 | 27.5 | 21.6 |
| Temporal | | | | | | | |
| Ctx | | | | | | | |
| AD 3 | 9.5 | 9.8 | 6.0 | 11.7 | 11.3 | 9.0 | 5.7 |
| Temporal | | | | | | | |
| Ctx | | | | | | | |
| AD 4 | 30.6 | 39.0 | 20.2 | 15.6 | 25.3 | 30.4 | 8.7 |
| Temporal | | | | | | | |
| Ctx | | | | | | | |
| AD 5 Inf | 45.4 | 37.1 | 39.2 | 43.8 | 36.3 | 41.8 | 73.7 |
| Temporal | | | | | | | |
| Ctx | | | | | | | |
| AD 5 Sup | 51.1 | 39.0 | 42.0 | 56.6 | 32.3 | 38.7 | 55.9 |
| Temporal | | | | | | | |
| Ctx | | | | | | | |
| AD 6 Inf | 38.2 | 59.9 | 49.3 | 40.9 | 46.7 | 47.6 | 76.8 |
| Temporal | | | | | | | |
| Ctx | | | | | | | |
| AD 6 Sup | 43.8 | 48.6 | 48.3 | 44.1 | 50.3 | 50.3 | 59.9 |
| Temporal | | | | | | | |
| Ctx | | | | | | | |
| Control 1 | 12.2 | 23.0 | 12.9 | 11.9 | 15.6 | 24.0 | 46.7 |
| Temporal | | | | | | | |
| Ctx | | | | | | | |
| Control 2 | 14.2 | 32.5 | 18.2 | 16.7 | 17.4 | 14.9 | 50.0 |
| Temporal | | | | | | | |
| Ctx | | | | | | | |
| Control 3 | 15.1 | 15.3 | 9.6 | 13.0 | 14.5 | 16.5 | 9.5 |
| Temporal | | | | | | | |
| Ctx | | | | | | | |
| Control 3 | 23.7 | 25.0 | 15.2 | 18.9 | 13.1 | 23.8 | 13.6 |
| Temporal | | | | | | | |
| Ctx | | | | | | | |
| Control | 26.1 | 47.0 | 27.0 | 32.5 | 30.6 | 39.8 | 46.0 |
| (Path) 1 | | | | | | | |
| Temporal | | | | | | | |
| Ctx | | | | | | | |
| Control | 24.5 | 25.9 | 16.0 | 19.5 | 20.4 | 24.8 | 0.0 |
| (Path) 2 | | | | | | | |
| Temporal | | | | | | | |
| Ctx | | | | | | | |
| Control | 11.7 | 16.0 | 7.5 | 12.9 | 10.9 | 11.9 | 31.0 |
| (Path) 3 | | | | | | | |
| Temporal | | | | | | | |
| Ctx | | | | | | | |
| Control | 21.9 | 27.4 | 17.1 | 19.8 | 18.2 | 21.6 | 39.5 |
| (Path) 4 | | | | | | | |
| Temporal | | | | | | | |
| Ctx | | | | | | | |
| AD 1 | 16.0 | 11.9 | 10.2 | 16.2 | 11.5 | 16.0 | 6.3 |
| Occipital Ctx | | | | | | | |
| AD 2 | 0.0 | 0.0 | 0.0 | 0.0 | 0.0 | 0.0 | 0.0 |
| Occipital Ctx | | | | | | | |
| (Missing) | | | | | | | |
| AD 3 | 10.7 | 6.0 | 6.4 | 11.7 | 8.8 | 10.2 | 4.9 |
| Occipital Ctx | | | | | | | |
| AD 4 | 18.9 | 23.7 | 13.0 | 12.6 | 17.9 | 18.6 | 11.1 |
| Occipital Ctx | | | | | | | |
| AD 5 | 24.8 | 28.3 | 25.3 | 16.7 | 22.5 | 22.7 | 42.3 |
| Occipital Ctx | | | | | | | |
| AD 6 | 20.6 | 31.9 | 20.2 | 17.8 | 17.0 | 22.1 | 14.8 |
| Occipital Ctx | | | | | | | |
| Control 1 | 9.5 | 14.4 | 6.0 | 11.3 | 8.7 | 7.2 | 8.8 |
| Occipital Ctx | | | | | | | |
| Control 2 | 31.9 | 42.6 | 26.4 | 24.8 | 33.2 | 29.3 | 82.4 |
| Occipital Ctx | | | | | | | |
| Control 3 | 18.8 | 13.0 | 10.7 | 16.4 | 17.1 | 19.2 | 8.8 |
| Occipital Ctx | | | | | | | |
| Control 4 | 18.2 | 17.0 | 12.0 | 12.1 | 12.6 | 13.6 | 24.0 |
| Occipital Ctx | | | | | | | |
| Control | 38.2 | 52.5 | 35.6 | 32.8 | 36.1 | 39.5 | 100.0 |
| (Path) 1 | | | | | | | |
| Occipital Ctx | | | | | | | |
| Control | 9.6 | 14.1 | 6.7 | 9.6 | 7.9 | 7.0 | 9.3 |
| (Path) 2 | | | | | | | |
| Occipital Ctx | | | | | | | |
| Control | 4.8 | 8.7 | 5.4 | 8.4 | 6.0 | 5.9 | 4.1 |
| (Path) 3 | | | | | | | |
| Occipital Ctx | | | | | | | |
| Control | 16.2 | 13.2 | 13.2 | 15.9 | 10.2 | 11.4 | 32.8 |
| (Path) 4 | | | | | | | |
| Occipital Ctx | | | | | | | |
| Control 1 | 14.4 | 21.9 | 8.8 | 15.2 | 16.3 | 15.7 | 9.2 |
| Parietal Ctx | | | | | | | |
| Control 2 | 32.8 | 28.9 | 34.4 | 39.5 | 28.3 | 37.1 | 28.1 |
| Parietal Ctx | | | | | | | |
| Control 3 | 20.6 | 19.8 | 11.5 | 14.5 | 8.7 | 10.8 | 9.1 |
| Parietal Ctx | | | | | | | |
| Control | 35.4 | 62.4 | 34.2 | 33.4 | 39.2 | 37.9 | 69.3 |
| (Path) 1 | | | | | | | |
| Parietal Ctx | | | | | | | |
| Control | 22.1 | 23.8 | 19.6 | 20.0 | 22.5 | 18.7 | 37.6 |
| (Path) 2 | | | | | | | |
| Parietal Ctx | | | | | | | |
| Control | 11.2 | 15.4 | 3.9 | 15.0 | 7.1 | 12.0 | 10.4 |
| (Path) 3 | | | | | | | |
| Parietal Ctx | | | | | | | |
| Control | 31.2 | 34.2 | 24.8 | 28.3 | 8.8 | 27.9 | 27.5 |
| (Path) 4 | | | | | | | |
| Parietal Ctx | | | | | | | |

TABLE ASL
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 13.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro; met) SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE ASM
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro; met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE ASN
General_screening_panel_v1.6

| | Rel. | Rel. | Rel. | Rel. | Rel. | Rel. | Rel. | Rel. | Rel. |
|---|---|---|---|---|---|---|---|---|---|
| | Exp. (%) | Exp. (%) | Exp. (%) | Exp. (%) | Exp. (%) | Exp. (%) | Exp. (%) | Exp. (%) | Exp. (%) |
| | Ag6413, | Ag6424, | Ag6425, | Ag6428, | Ag6430, | Ag6431, | Ag6431, | Ag6439, | Ag6964, |
| Tissue | Run | Run | Run | Run | Run | Run | Run | Run | Run |
| Name | 277249371 | 277221719 | 277221721 | 277222439 | 277222443 | 277633568 | 278389390 | 277223175 | 278388946 |
| Adipose | 25.9 | 0.0 | 2.6 | 20.0 | 8.2 | 17.4 | 13.8 | 17.3 | 18.8 |
| Melanoma* | 0.5 | 0.0 | 0.0 | 2.0 | 0.5 | 0.8 | 0.9 | 0.4 | 0.7 |
| Hs688(A).T | | | | | | | | | |
| Melanoma* | 2.7 | 0.0 | 0.2 | 4.1 | 0.6 | 2.5 | 2.2 | 2.9 | 2.4 |
| Hs688(B).T | | | | | | | | | |
| Melanoma* | 0.3 | 0.0 | 0.0 | 0.7 | 0.7 | 0.4 | 0.4 | 0.4 | 0.7 |
| M14 | | | | | | | | | |
| Melanoma* | 0.0 | 0.0 | 0.0 | 0.1 | 0.0 | 0.0 | 0.0 | 0.0 | 0.1 |
| LOXIMVI | | | | | | | | | |
| Melanoma* | 15.2 | 0.0 | 2.2 | 30.4 | 22.5 | 18.2 | 14.6 | 18.3 | 15.9 |
| SK-MEL-5 | | | | | | | | | |
| Squamous | 0.0 | 0.0 | 0.0 | 0.1 | 0.3 | 0.1 | 0.2 | 0.0 | 0.1 |
| cell | | | | | | | | | |
| carcinoma | | | | | | | | | |
| SCC-4 | | | | | | | | | |
| Testis Pool | 5.2 | 0.0 | 3.5 | 8.8 | 4.2 | 10.4 | 9.0 | 9.1 | 9.9 |
| Prostate ca.* | 1.9 | 0.0 | 0.5 | 2.5 | 1.0 | 1.9 | 1.8 | 1.3 | 4.3 |
| (bone met) | | | | | | | | | |
| PC-3 | | | | | | | | | |
| Prostate Pool | 8.1 | 0.0 | 1.0 | 11.5 | 8.5 | 11.3 | 12.1 | 28.5 | 10.0 |
| Placenta | 0.5 | 0.0 | 0.0 | 0.7 | 0.1 | 0.1 | 0.1 | 0.5 | 0.4 |
| Uterus Pool | 2.2 | 0.0 | 1.5 | 4.5 | 2.6 | 4.6 | 4.5 | 5.3 | 4.1 |
| Ovarian ca. | 0.9 | 0.0 | 0.3 | 1.1 | 0.8 | 0.7 | 1.1 | 1.6 | 4.0 |
| OVCAR-3 | | | | | | | | | |
| Ovarian ca. | 0.8 | 0.0 | 0.2 | 1.7 | 1.5 | 0.8 | 0.9 | 1.3 | 1.7 |
| SK-OV-3 | | | | | | | | | |
| Ovarian ca. | 0.2 | 0.0 | 0.0 | 0.9 | 0.5 | 0.4 | 0.8 | 0.9 | 0.5 |
| OVCAR-4 | | | | | | | | | |
| Ovarian ca. | 1.6 | 0.0 | 1.3 | 2.9 | 1.5 | 1.3 | 1.7 | 1.4 | 7.9 |
| OVCAR-5 | | | | | | | | | |
| Ovarian ca. | 100.0 | 100.0 | 100.0 | 77.9 | 90.8 | 84.7 | 97.9 | 69.3 | 75.8 |
| IGROV-1 | | | | | | | | | |
| Ovarian ca. | 13.6 | 5.6 | 21.9 | 14.0 | 11.9 | 15.6 | 14.6 | 17.3 | 16.7 |
| OVCAR-8 | | | | | | | | | |
| Ovary | 2.7 | 0.0 | 0.3 | 5.2 | 2.1 | 3.1 | 2.3 | 2.8 | 2.4 |
| Breast ca. | 0.3 | 0.0 | 0.0 | 0.3 | 0.4 | 0.1 | 0.2 | 0.5 | 0.5 |
| MCF-7 | | | | | | | | | |
| Breast ca. | 0.1 | 0.0 | 0.0 | 0.4 | 0.4 | 0.2 | 0.2 | 0.2 | 0.3 |
| MDA-MB- | | | | | | | | | |
| 231 | | | | | | | | | |
| Breast ca. | 0.5 | 0.0 | 0.0 | 0.5 | 0.3 | 0.1 | 0.5 | 0.6 | 0.4 |
| BT 549 | | | | | | | | | |
| Breast ca. | 0.0 | 0.0 | 0.0 | 0.5 | 0.3 | 0.2 | 0.3 | 0.4 | 0.5 |
| T47D | | | | | | | | | |
| Breast ca. | 0.6 | 0.0 | 0.0 | 0.7 | 0.7 | 0.6 | 0.6 | 0.6 | 0.8 |
| MDA-N | | | | | | | | | |
| Breast Pool | 15.0 | 0.0 | 4.1 | 21.8 | 19.5 | 14.6 | 10.7 | 12.2 | 16.7 |
| Trachea | 4.5 | 0.0 | 0.7 | 8.4 | 2.9 | 4.8 | 4.2 | 4.7 | 5.6 |
| Lung | 2.8 | 0.0 | 0.7 | 2.3 | 1.3 | 4.2 | 3.2 | 3.9 | 5.1 |
| Fetal Lung | 3.9 | 0.0 | 0.3 | 9.1 | 4.0 | 5.0 | 4.8 | 5.3 | 6.1 |
| Lung ca. | 2.0 | 2.0 | 0.9 | 3.5 | 2.7 | 3.3 | 2.6 | 4.0 | 2.3 |
| NCI-N417 | | | | | | | | | |
| Lung ca. | 3.5 | 3.1 | 2.7 | 6.5 | 7.0 | 5.0 | 3.5 | 4.9 | 44.1 |
| LX-1 | | | | | | | | | |
| Lung ca. | 0.1 | 0.0 | 0.0 | 0.3 | 0.5 | 0.1 | 0.2 | 0.1 | 0.1 |
| NCI-H146 | | | | | | | | | |
| Lung ca. | 4.0 | 2.3 | 0.4 | 6.8 | 6.3 | 5.3 | 4.5 | 4.5 | 3.8 |
| SHP-77 | | | | | | | | | |
| Lung ca. | 0.3 | 0.0 | 2.6 | 0.9 | 0.3 | 0.0 | 0.4 | 0.6 | 4.7 |
| A549 | | | | | | | | | |
| Lung ca. | 0.2 | 0.0 | 0.0 | 0.9 | 0.7 | 0.6 | 0.3 | 0.4 | 0.5 |
| NCI-H526 | | | | | | | | | |
| Lung ca. | 2.9 | 0.0 | 1.0 | 4.6 | 4.5 | 4.8 | 3.2 | 2.9 | 10.3 |
| NCI-H23 | | | | | | | | | |
| Lung ca. | 0.0 | 0.0 | 0.0 | 0.2 | 0.2 | 0.1 | 0.3 | 0.0 | 0.3 |
| NCI-H460 | | | | | | | | | |
| Lung ca. | 0.5 | 0.0 | 0.0 | 0.5 | 0.6 | 1.0 | 0.6 | 0.5 | 0.7 |
| HOP-62 | | | | | | | | | |
| Lung ca. | 1.7 | 0.0 | 0.6 | 2.3 | 2.4 | 1.7 | 1.3 | 3.3 | 8.9 |
| NCI-H522 | | | | | | | | | |
| Liver | 0.1 | 0.0 | 0.0 | 0.0 | 0.1 | 0.0 | 0.0 | 0.1 | 2.0 |
| Fetal Liver | 0.3 | 0.0 | 0.3 | 1.1 | 0.6 | 0.6 | 0.5 | 0.8 | 8.2 |
| Liver ca. | 0.1 | 0.0 | 0.3 | 0.2 | 0.1 | 0.0 | 0.2 | 0.1 | 2.4 |
| HepG2 | | | | | | | | | |
| Kidney Pool | 27.9 | 6.5 | 0.0 | 47.0 | 34.9 | 33.9 | 28.1 | 43.2 | 32.8 |
| Fetal Kidney | 1.4 | 0.0 | 0.0 | 4.9 | 5.1 | 4.1 | 4.0 | 5.8 | 11.5 |
| Renal ca. | 0.2 | 0.0 | 0.0 | 0.2 | 0.2 | 0.3 | 0.1 | 0.3 | 0.9 |
| 786-0 | | | | | | | | | |
| Renal ca. | 0.0 | 0.0 | 1.8 | 0.2 | 0.1 | 0.0 | 0.3 | 0.5 | 8.5 |
| A498 | | | | | | | | | |
| Renal ca. | 1.5 | 0.0 | 0.5 | 2.5 | 0.7 | 1.7 | 1.5 | 1.2 | 2.5 |
| ACHN | | | | | | | | | |
| Renal ca. | 0.3 | 0.0 | 0.0 | 0.5 | 0.3 | 0.2 | 0.2 | 0.6 | 0.3 |
| UO-31 | | | | | | | | | |
| Renal ca. | 1.9 | 0.0 | 0.4 | 3.1 | 2.5 | 2.0 | 1.9 | 2.1 | 4.6 |
| TK-10 | | | | | | | | | |
| Bladder | 4.2 | 0.0 | 0.0 | 5.9 | 3.0 | 5.5 | 5.1 | 8.3 | 6.7 |
| Gastric ca. | 0.9 | 0.0 | 0.0 | 1.7 | 1.7 | 0.9 | 1.2 | 1.1 | 6.7 |
| (liver met.) | | | | | | | | | |
| NCI-N87 | | | | | | | | | |
| Gastric ca. | 0.4 | 0.0 | 0.5 | 0.8 | 0.4 | 0.2 | 0.3 | 0.4 | 0.9 |
| KATO III | | | | | | | | | |
| Colon ca. | 0.0 | 0.0 | 1.5 | 0.2 | 0.0 | 0.2 | 0.2 | 0.3 | 1.2 |
| SW-948 | | | | | | | | | |
| Colon ca. | 20.9 | 9.5 | 5.2 | 41.8 | 39.0 | 27.0 | 23.3 | 23.0 | 33.7 |
| SW480 | | | | | | | | | |
| Colon ca.* | 13.3 | 7.7 | 4.8 | 16.4 | 15.5 | 12.8 | 10.3 | 6.1 | 25.0 |
| (SW480 | | | | | | | | | |
| met) SW620 | | | | | | | | | |
| Colon ca. | 0.2 | 0.0 | 0.0 | 0.0 | 0.0 | 0.2 | 0.2 | 0.0 | 0.3 |
| HT29 | | | | | | | | | |
| Colon ca. | 2.1 | 1.6 | 0.2 | 3.2 | 3.8 | 2.5 | 2.0 | 2.1 | 4.3 |
| HCT-116 | | | | | | | | | |
| Colon ca. | 15.0 | 10.4 | 3.6 | 27.0 | 22.2 | 19.1 | 16.7 | 18.3 | 38.2 |
| CaCo-2 | | | | | | | | | |
| Colon cancer | 9.0 | 0.0 | 3.3 | 11.0 | 6.5 | 11.9 | 7.6 | 7.7 | 20.4 |
| tissue | | | | | | | | | |
| Colon ca. | 1.3 | 0.0 | 3.0 | 2.5 | 1.7 | 2.0 | 1.5 | 1.8 | 6.0 |
| SW1116 | | | | | | | | | |
| Colon ca. | 0.1 | 0.0 | 0.4 | 0.3 | 0.2 | 0.2 | 0.0 | 0.2 | 0.8 |
| Colo-205 | | | | | | | | | |
| Colon ca. | 0.8 | 0.0 | 3.6 | 1.4 | 1.3 | 1.5 | 1.5 | 1.4 | 2.6 |
| SW-48 | | | | | | | | | |
| Colon Pool | 20.3 | 0.0 | 5.0 | 28.1 | 28.7 | 23.2 | 18.7 | 25.5 | 20.6 |
| Small | 14.0 | 0.0 | 1.7 | 17.1 | 10.5 | 11.2 | 13.0 | 12.8 | 10.4 |
| Intestine | | | | | | | | | |
| Pool | | | | | | | | | |
| Stomach | 8.1 | 0.0 | 2.3 | 14.3 | 6.2 | 9.5 | 9.3 | 8.5 | 10.7 |
| Pool | | | | | | | | | |
| Bone | 6.8 | 0.0 | 1.6 | 14.3 | 11.3 | 10.2 | 8.7 | 18.7 | 12.5 |
| Marrow Pool | | | | | | | | | |
| Fetal Heart | 10.1 | 0.0 | 2.3 | 25.5 | 24.3 | 24.5 | 21.8 | 33.7 | 20.7 |
| Heart Pool | 28.7 | 5.2 | 7.0 | 29.7 | 23.0 | 25.9 | 17.2 | 33.7 | 26.1 |
| Lymph Node | 17.6 | 0.0 | 6.1 | 33.7 | 30.4 | 22.1 | 23.7 | 19.9 | 24.7 |
| Pool | | | | | | | | | |
| Fetal | 31.9 | 36.9 | 5.2 | 54.3 | 46.7 | 48.6 | 46.3 | 19.1 | 50.7 |
| Skeletal | | | | | | | | | |
| Muscle | | | | | | | | | |
| Skeletal | 17.4 | 12.3 | 9.2 | 29.3 | 21.5 | 29.5 | 25.9 | 22.1 | 32.3 |
| Muscle Pool | | | | | | | | | |
| Spleen Pool | 0.9 | 0.0 | 0.0 | 1.9 | 2.0 | 2.0 | 1.7 | 2.7 | 3.1 |
| Thymus | 4.4 | 0.0 | 2.0 | 10.4 | 7.5 | 8.1 | 9.4 | 7.7 | 7.0 |
| Pool | | | | | | | | | |
| CNS cancer | 9.8 | 1.6 | 1.5 | 14.9 | 6.1 | 10.7 | 10.0 | 10.9 | 14.1 |
| (glio/astro) | | | | | | | | | |
| U87-MG | | | | | | | | | |
| CNS cancer | 3.5 | 0.0 | 0.3 | 4.7 | 2.9 | 3.8 | 3.1 | 3.8 | 5.8 |
| (glio/astro) | | | | | | | | | |
| U-118-MG | | | | | | | | | |
| CNS cancer | 1.9 | 0.0 | 0.0 | 2.6 | 1.7 | 2.1 | 1.0 | 1.4 | 2.6 |
| (neuro; met) | | | | | | | | | |
| SK-N-AS | | | | | | | | | |
| CNS cancer | 0.1 | 0.0 | 0.0 | 0.0 | 0.2 | 0.1 | 0.2 | 0.1 | 0.1 |
| (astro) SF- | | | | | | | | | |
| 539 | | | | | | | | | |
| CNS cancer | 8.1 | 1.9 | 1.1 | 14.9 | 5.9 | 6.5 | 10.0 | 11.7 | 9.7 |
| (astro) SNB- | | | | | | | | | |
| 75 | | | | | | | | | |
| CNS cancer | 79.6 | 84.1 | 79.0 | 100.0 | 100.0 | 100.0 | 100.0 | 100.0 | 100.0 |
| (glio) SNB- | | | | | | | | | |
| 19 | | | | | | | | | |
| CNS cancer | 8.2 | 1.8 | 0.0 | 11.3 | 9.0 | 8.0 | 7.8 | 8.2 | 14.8 |
| (glio) SF- | | | | | | | | | |
| 295 | | | | | | | | | |
| Brain | 3.7 | 2.3 | 0.8 | 7.7 | 6.9 | 6.2 | 4.8 | 8.0 | 5.3 |
| (Amygdala) | | | | | | | | | |
| Pool | | | | | | | | | |
| Brain | 12.0 | 6.6 | 0.4 | 19.8 | 11.1 | 10.7 | 9.7 | 8.8 | 9.7 |
| (cerebellum) | | | | | | | | | |
| Brain (fetal) | 4.2 | 3.0 | 0.7 | 12.7 | 11.5 | 6.6 | 5.6 | 6.8 | 6.4 |
| Brain | 7.5 | 3.1 | 3.2 | 11.7 | 11.0 | 8.6 | 6.9 | 11.03 | 10.2 |
| (Hippocam- | | | | | | | | | |
| pus) Pool | | | | | | | | | |
| Cerebral | 9.7 | 1.7 | 0.6 | 11.0 | 7.5 | 7.5 | 0.7 | 11.6 | 8.7 |
| Cortex Pool | | | | | | | | | |
| Brain | 7.4 | 1.8 | 2.2 | 11.7 | 8.5 | 10.4 | 4.7 | 10.0 | 9.3 |
| (Substantia | | | | | | | | | |
| nigra) Pool | | | | | | | | | |
| Brain | 7.6 | 0.0 | 2.7 | 13.2 | 10.0 | 9.3 | 0.2 | 9.7 | 8.7 |
| (Thalamus) | | | | | | | | | |
| Pool | | | | | | | | | |
| Brain | 6.1 | 0.0 | 0.4 | 10.6 | 8.0 | 5.8 | 0.3 | 5.6 | 8.7 |
| (whole) | | | | | | | | | |
| Spinal Cord | 10.1 | 3.2 | 2.3 | 14.7 | 12.8 | 11.0 | 7.6 | 12.2 | 9.0 |
| Pool | | | | | | | | | |
| Adrenal | 3.5 | 0.0 | 0.3 | 9.9 | 6.1 | 3.9 | 3.7 | 4.8 | 4.1 |
| Gland | | | | | | | | | |
| Pituitary | 0.9 | 0.0 | 0.0 | 1.1 | 0.8 | 1.2 | 1.1 | 1.4 | 0.5 |
| gland Pool | | | | | | | | | |
| Salivary | 0.9 | 0.0 | 0.0 | 1.8 | 1.1 | 1.3 | 0.9 | 1.1 | 1.0 |
| Gland | | | | | | | | | |
| Thyroid | 2.0 | 0.0 | 0.3 | 3.1 | 0.8 | 2.5 | 2.5 | 1.9 | 2.3 |
| (female) | | | | | | | | | |
| Pancreatic | 0.5 | 0.0 | 0.0 | 0.8 | 0.8 | 0.7 | 0.6 | 0.7 | 2.2 |
| ca. CAPAN2 | | | | | | | | | |
| Pancreas | 1.2 | 0.0 | 0.0 | 2.0 | 1.1 | 1.1 | 1.6 | 3.2 | 2.3 |
| Pool | | | | | | | | | |

TABLE ASO
Panel 4.1D

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag4983, | Ag6413, | Ag6425, | Ag6428, | Ag6431, | Ag6439, |
| | Run | Run | Run | Run | Run | Run |
| Tissue Name | 218623570 | 269239947 | 268713999 | 268767535 | 268767577 | 268760823 |
| Secondary Th1 act | 0.1 | 0.3 | 0.0 | 1.3 | 0.7 | 0.0 |
| Secondary Th2 act | 0.5 | 0.3 | 0.0 | 1.2 | 0.8 | 0.0 |
| Secondary Tr1 act | 0.0 | 0.0 | 0.0 | 0.0 | 0.7 | 0.0 |
| Secondary Th1 rest | 0.1 | 0.0 | 0.0 | 0.0 | 0.0 | 0.0 |
| Secondary Th2 rest | 0.3 | 0.0 | 0.0 | 0.0 | 0.0 | 0.0 |
| Secondary Tr1 rest | 0.1 | 0.3 | 0.0 | 0.4 | 0.0 | 0.0 |
| Primary Th1 act | 0.0 | 0.0 | 0.0 | 0.0 | 0.0 | 0.0 |
| Primary Th2 act | 0.2 | 0.4 | 0.0 | 0.3 | 0.4 | 0.0 |
| Primary Tr1 act | 0.1 | 0.0 | 0.0 | 0.7 | 0.7 | 0.0 |
| Primary Th1 rest | 0.0 | 0.0 | 0.0 | 0.1 | 0.3 | 1.2 |
| Primary Th2 rest | 0.0 | 0.0 | 0.0 | 0.4 | 0.2 | 0.0 |
| Primary Tr1 rest | 0.3 | 0.0 | 0.0 | 0.0 | 0.0 | 0.0 |
| CD45RA CD4 | 0.4 | 2.8 | 0.0 | 5.4 | 2.4 | 2.6 |
| lymphocyte act | | | | | | |
| CD45RO CD4 | 0.1 | 2.2 | 0.0 | 1.5 | 0.7 | 2.3 |
| lymphocyte act | | | | | | |
| CD8 lymphocyte act | 0.4 | 0.9 | 0.0 | 0.7 | 0.0 | 0.0 |
| Secondary CD8 | 0.1 | 0.0 | 0.0 | 8.8 | 0.0 | 0.0 |
| lymphocyte rest | | | | | | |
| Secondary CD8 | 0.0 | 0.1 | 0.0 | 0.4 | 0.3 | 0.0 |
| lymphocyte act | | | | | | |
| CD4 lymphocyte | 0.1 | 0.0 | 0.0 | 0.5 | 0.4 | 0.0 |
| none | | | | | | |
| 2ry | 0.3 | 0.2 | 0.0 | 0.0 | 0.0 | 1.2 |
| Th1/Th2/Tr1_anti- | | | | | | |
| CD95 CH11 | | | | | | |
| LAK cells rest | 5.6 | 5.0 | 2.7 | 11.8 | 3.8 | 15.2 |
| LAK cells IL-2 | 0.4 | 0.3 | 0.0 | 0.0 | 0.0 | 0.0 |
| LAK cells IL-2 + | 0.2 | 0.0 | 0.0 | 0.0 | 0.0 | 0.0 |
| IL-12 | | | | | | |
| LAK cells IL-2 + | 0.1 | 0.3 | 0.0 | 0.0 | 0.0 | 0.0 |
| IFN gamma | | | | | | |
| LAK cells IL-2 + | 0.0 | 0.0 | 0.0 | 0.0 | 0.0 | 0.0 |
| IL-18 | | | | | | |
| LAK cells | 4.5 | 4.0 | 15.7 | 15.1 | 6.3 | 9.0 |
| PMA/ionomycin | | | | | | |
| NK Cells IL-2 rest | 0.9 | 0.1 | 0.0 | 3.4 | 2.5 | 1.4 |
| Two Way MLR 3 | 1.4 | 1.1 | 0.0 | 2.2 | 1.3 | 1.4 |
| day | | | | | | |
| Two Way MLR 5 | 4.5 | 0.9 | 0.0 | 0.8 | 0.9 | 0.0 |
| day | | | | | | |
| Two Way MLR 7 | 2.3 | 0.7 | 13.2 | 1.1 | 2.6 | 3.7 |
| day | | | | | | |
| PBMC rest | 0.1 | 0.0 | 0.0 | 0.0 | 0.0 | 0.0 |
| PBMC PWM | 0.6 | 0.0 | 0.0 | 1.3 | 0.0 | 0.0 |
| PBMC PHA-L | 0.3 | 0.2 | 0.0 | 0.6 | 0.7 | 0.0 |
| Ramos (B cell) none | 0.1 | 0.0 | 0.0 | 0.0 | 0.0 | 0.0 |
| Ramos (B cell) | 0.0 | 0.0 | 0.0 | 0.7 | 0.2 | 0.0 |
| ionomycin | | | | | | |
| B lymphocytes | 0.5 | 0.0 | 0.0 | 0.0 | 0.0 | 0.0 |
| PWM | | | | | | |
| B lymphocytes | 0.2 | 0.0 | 0.0 | 0.9 | 0.0 | 0.0 |
| CD40L and IL-4 | | | | | | |
| EOL-1 dbcAMP | 3.7 | 2.6 | 9.1 | 29.1 | 8.1 | 68.8 |
| EOL-1 dbcAMP | 1.6 | 0.7 | 0.0 | 0.0 | 2.7 | 1.8 |
| PMA/ionomycin | | | | | | |
| Dendritic cells none | 5.6 | 3.1 | 13.8 | 4.1 | 5.3 | 0.0 |
| Dendritic cells LPS | 1.6 | 0.3 | 0.0 | 1.0 | 0.7 | 0.0 |
| Dendritic cells anti- | 2.0 | 1.6 | 3.3 | 0.5 | 0.2 | 0.0 |
| CD40 | | | | | | |
| Monocytes rest | 0.2 | 0.0 | 0.0 | 0.4 | 0.0 | 0.0 |
| Monocytes LPS | 2.2 | 3.3 | 0.0 | 5.7 | 1.8 | 2.6 |
| Macrophages rest | 0.9 | 1.8 | 0.0 | 0.6 | 0.6 | 0.0 |
| Macrophages LPS | 7.5 | 4.0 | 0.0 | 5.4 | 6.3 | 9.2 |
| HUVEC none | 0.1 | 0.0 | 0.0 | 0.0 | 0.0 | 0.0 |
| HUVEC starved | 0.0 | 0.0 | 0.0 | 0.0 | 0.3 | 0.0 |
| HUVEC IL-1beta | 0.0 | 0.0 | 0.0 | 0.0 | 0.5 | 0.0 |
| HUVEC IFN | 0.2 | 0.0 | 0.0 | 0.0 | 0.0 | 0.0 |
| gamma | | | | | | |
| HUVEC TNF | 0.0 | 0.0 | 0.0 | 0.0 | 0.0 | 0.0 |
| alpha + IFN gamma | | | | | | |
| HUVEC TNF | 0.6 | 0.0 | 0.0 | 0.0 | 0.4 | 0.0 |
| alpha + IL4 | | | | | | |
| HUVEC IL-11 | 0.0 | 0.0 | 0.0 | 0.4 | 0.3 | 0.0 |
| Lung Microvascular | 0.2 | 0.3 | 0.0 | 0.4 | 0.0 | 0.0 |
| EC none | | | | | | |
| Lung Microvascular | 0.1 | 0.0 | 0.0 | 0.0 | 0.0 | 0.0 |
| EC TNFalpha + IL- | | | | | | |
| 1beta | | | | | | |
| Microvascular | 0.1 | 0.0 | 0.0 | 0.0 | 0.0 | 0.0 |
| Dermal EC none | | | | | | |
| Microsvasular | 0.1 | 0.0 | 0.0 | 0.0 | 0.0 | 0.0 |
| Dermal EC | | | | | | |
| TNFalpha + IL- | | | | | | |
| 1beta | | | | | | |
| Bronchial | 0.0 | 0.0 | 0.0 | 0.0 | 0.0 | 0.0 |
| epithelium | | | | | | |
| TNFalpha + IL1beta | | | | | | |
| Small airway | 0.0 | 0.0 | 0.0 | 0.0 | 0.0 | 0.0 |
| epithelium none | | | | | | |
| Small airway | 0.3 | 0.0 | 0.0 | 0.0 | 0.0 | 0.0 |
| epithelium | | | | | | |
| TNFalpha + IL- | | | | | | |
| 1beta | | | | | | |
| Coronery artery | 0.1 | 0.6 | 0.0 | 0.0 | 0.0 | 0.0 |
| SMC rest | | | | | | |
| Coronery artery | 0.4 | 0.9 | 6.2 | 0.3 | 1.5 | 0.0 |
| SMC TNFalpha + | | | | | | |
| IL-1beta | | | | | | |
| Astrocytes rest | 67.8 | 97.3 | 100.0 | 100.0 | 100.0 | 100.0 |
| Astrocytes | 100.0 | 100.0 | 74.2 | 97.3 | 74.7 | 95.9 |
| TNFalpha + IL- | | | | | | |
| 1beta | | | | | | |
| KU-812 (Basophil) | 0.1 | 0.0 | 0.0 | 0.0 | 0.4 | 0.0 |
| rest | | | | | | |
| KU-812 (Basophil) | 0.0 | 0.0 | 0.0 | 0.0 | 0.0 | 0.0 |
| PMA/ionomycin | | | | | | |
| CCD1106 | 0.2 | 0.0 | 0.0 | 0.0 | 0.8 | 0.0 |
| (Keratinocytes) none | | | | | | |
| CCD1106 | 0.3 | 0.0 | 0.0 | 0.0 | 0.0 | 0.0 |
| (Keratinocytes) | | | | | | |
| TNFalpha + IL- | | | | | | |
| 1beta | | | | | | |
| Liver cirrhosis | 2.3 | 7.2 | 4.6 | 2.6 | 6.7 | 8.5 |
| NCI-H292 none | 0.3 | 0.3 | 0.0 | 1.7 | 0.6 | 0.0 |
| NCI-H292 IL-4 | 0.3 | 0.0 | 0.0 | 0.0 | 0.5 | 0.0 |
| NCI-H292 IL-9 | 0.3 | 0.0 | 0.0 | 0.7 | 0.5 | 0.0 |
| NCI-H292 IL-13 | 0.6 | 0.6 | 0.0 | 0.9 | 0.9 | 0.0 |
| NCI-H292 IFN | 0.2 | 0.0 | 0.0 | 0.5 | 0.6 | 0.0 |
| gamma | | | | | | |
| HPAEC none | 0.0 | 0.3 | 0.0 | 0.0 | 0.0 | 0.0 |
| HPAEC TNF | 0.0 | 0.3 | 0.0 | 0.0 | 0.0 | 0.0 |
| alpha + IL-1 beta | | | | | | |
| Lung fibroblast none | 29.7 | 62.9 | 31.4 | 95.9 | 65.5 | 94.0 |
| Lung fibroblast TNF | 16.0 | 36.9 | 22.2 | 48.6 | 39.8 | 62.9 |
| alpha + IL-1 beta | | | | | | |
| Lung fibroblast IL-4 | 26.1 | 28.7 | 19.1 | 27.4 | 21.2 | 34.9 |
| Lung fibroblast IL-9 | 28.5 | 42.0 | 23.5 | 24.0 | 26.8 | 96.6 |
| Lung fibroblast IL- | 31.6 | 14.6 | 4.5 | 11.9 | 10.4 | 13.4 |
| 13 | | | | | | |
| Lung fibroblast IFN | 20.4 | 32.8 | 15.7 | 55.9 | 46.3 | 89.5 |
| gamma | | | | | | |
| Dermal fibroblast | 2.5 | 2.9 | 0.0 | 6.0 | 6.3 | 4.1 |
| CCD1070 rest | | | | | | |
| Dermal fibroblast | 1.1 | 1.3 | 0.0 | 2.7 | 0.8 | 2.3 |
| CCD1070 TNF | | | | | | |
| alpha | | | | | | |
| Dermal fibroblast | 1.9 | 2.9 | 0.0 | 5.6 | 1.3 | 0.0 |
| CCD1070 IL-1 beta | | | | | | |
| Dermal fibroblast | 9.3 | 20.3 | 8.5 | 30.6 | 20.2 | 26.6 |
| IFN gamma | | | | | | |
| Dermal fibroblast | 10.7 | 14.6 | 4.1 | 30.8 | 19.8 | 25.5 |
| IL-4 | | | | | | |
| Dermal Fibroblasts | 24.8 | 42.3 | 8.0 | 54.3 | 46.7 | 47.3 |
| rest | | | | | | |
| Neutrophils | 0.7 | 0.0 | 0.0 | 0.9 | 0.4 | 0.0 |
| TNFa + LPS | | | | | | |
| Neutrophils rest | 0.1 | 0.0 | 0.0 | 0.0 | 0.3 | 0.0 |
| Colon | 7.9 | 4.7 | 4.0 | 4.6 | 9.5 | 8.4 |
| Lung | 2.2 | 1.2 | 0.0 | 2.8 | 4.6 | 2.1 |
| Thymus | 3.1 | 0.8 | 0.0 | 0.0 | 0.4 | 2.4 |
| Kidney | 4.2 | 4.4 | 4.9 | 7.8 | 9.7 | 5.2 |

TABLE ASP
general oncology screening panel_v_2.4

| | Rel. Exp. (%) | Rel. Exp. (%) |
|---|---|---|
| | Ag4983, Run | Ag6442, Run |
| Tissue Name | 260281959 | 264979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell carcinoma 3 | 5.6 | 8.3 |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate adenocarcinoma 1 | 9.2 | 7.5 |
| Prostate adenocarcinoma 2 | 3.5 | 8.0 |
| Prostate adenocarcinoma 3 | 14.3 | 9.0 |
| Prostate adenocarcinoma 4 | 16.4 | 9.1 |
| Prostate NAT 5 | 16.8 | 9.9 |
| Prostate adenocarcinoma 6 | 3.2 | 7.7 |
| Prostate adenocarcinoma 7 | 9.2 | 17.3 |
| Prostate adenocarcinoma 8 | 3.0 | 0.0 |
| Prostate adenocarcinoma 9 | 27.0 | 33.9 |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0

Summary: Ag4983/Ag6413/Ag6428/Ag6430/Ag6431/Ag6439/Ag6442 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6424/Ag6425 Expression of this gene is low/undetectable (CTs>35) across all of the samples on this panel (data not shown).

General_screening_panel_v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal, breast, ovarian, prostate, melanoma and brain cancers. Thus, expression of this gene-could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening_panel_v1.6 Summary: Ag6413/Ag6424/Ag6425/Ag6428/Ag6430/Ag6431/Ag6439/Ag6442 Eight experiments with seven different probe and primer sets are in very good agreement. Highest expression of this gene is detected in a ovarian cancer IGROV-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Panel 4.1D

Summary: Ag4983/Ag6413/Ag6428/Ag6430/Ag6431/Ag6439/Ag6442 Seven experiments with different probe and primer sets are in excellent agreement. Highest expression of this gene is detected in both resting and cytokine activated astrocytes (CTs=22-34.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

Ag6424 Expression of this gene is low/undetectable (CTs>35) across all of the samples on this panel (data not shown).

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AT. CG56054-13: Integrin Alpha 7-Like Protein.

Expression of gene CG56054-13 was assessed using the primer-probe sets Ag4983, Ag6442, Ag6424, Ag6425, Ag6428, Ag6430, Ag6431, Ag6440, Ag6446, Ag6413 and Ag6964, described in Tables ATA, ATB, ATC, ATD, ATE, ATF, ATG, ATH, ATI, ATJ and ATK. Results of the RTQ-PCR runs are shown in Tables ATL, ATM, ATN, ATO, ATP and ATQ.

TABLE ATA

Probe Name Ag4983

| | | | Start | |
|---|---|---|---|---|
| Primers | Sequences | Length | Position | SEQ ID No |
| Forward | 5′-ccaggtcaccttctacctcatc-3′ | 22 | 2330 | 600 |
| Probe | TET-5′-cttagcaccccgggatcagcatt-3′-TAMRA | 24 | 2352 | 601 |
| Reverse | 5′-aacagcagctctacctccagtt-3′ | 22 | 2386 | 602 |

TABLE ATB

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3′ | 22 | 2769 | 603 |
| Probe | TET-5′-ccacctgagcagcaggagcct-3′-TAMRA | 21 | 2808 | 604 |
| Reverse | 5′-gcgcagtccagggtg-3′ | 15 | 2894 | 605 |

TABLE ATC

Probe Name Ag6424

| | | | | |
|---|---|---|---|---|
| Primers | Sequences | Length | Start Position | SEQ ID No |

| | | | | |
|---|---|---|---|---|
| Forward | 5′-ttgggttctgccagca-3′ | 16 | 637 | 606 |
| Probe | TET-5′-cacagctgccgccttctccc-3′-TAMRA | 20 | 656 | 607 |
| Reverse | 5′-aaaagcaaccccttccaa-3′ | 18 | 719 | 608 |

TABLE ATD

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 3504 | 609 |
| Probe | TET-5′-catcccgagctgggcccc-3′-TAMRA | 18 | 3536 | 610 |
| Reverse | 5′-gccctggatgcccat-3′ | 15 | 3555 | 611 |

TABLE ATE

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1289 | 612 |
| Probe | TET-5′-ccttcacaggtgctggagggc-3′-TAMRA | 21 | 1329 | 613 |
| Reverse | 5′-agggagtagccgaagctct-3′ | 19 | 1366 | 614 |

TABLE ATE

Probe Name Ag6430

| | | | Start | |
|---|---|---|---|---|
| Primers | Sequences | Length | Position | SEQ ID No |
| Forward | 5′-gtgaccaacattgatagctcaga-3′ | 23 | 738 | 615 |
| Probe | TET-5′-ccccgagccagctggtgtataaaactttg3′ | 28 | 761 | 616 |
| Reverse | 5′-gggagccggtcagca-3′ | 15 | 794 | 617 |

TABLE ATG

Probe Name Ag6431

| | | | Start | |
|---|---|---|---|---|
| Primers | Sequences | Length | Position | SEQ ID No |
| Forward | 5′-aaacatcaccctggactgc-3′ | 19 | 2888 | 618 |
| Probe | TET-5′-tggtgttcagctgcccactctacag-3′-TAMRA | 25 | 2929 | 619 |
| Reverse | 5′-ccgcgcggtcaaa-3′ | 13 | 2955 | 620 |

TABLE ATH

Probe Name Ag6440

| | | | | |
|---|---|---|---|---|
| Primers | Sequences | Length | Start Position | SEQ ID No |

| | | | | |
|---|---|---|---|---|
| Forward | 5′-accatcctgaggaacaactg-3′ | 20 | 3461 | 621 |
| Probe | TET-5′-ctgacgggcatcccgagct-3′-TAMRA | 19 | 3528 | 622 |
| Reverse | 5′-ccctggatgcccatc-3′ | 15 | 3554 | 623 |

TABLE ATI

Probe Name Ag6446

| | | | Start | |
|---|---|---|---|---|
| Primers | Sequences | Length | Position | SEQ ID No |
| Forward | 5′-gcttcttccatcggagca-3′ | 18 | 3244 | 624 |
| Probe | TET-5′-caactatcaccgggcctgtctggc-3′-TAMRA | 24 | 3284 | 625 |
| Reverse | 5′-catggctgaaggctgca-3′ | 17 | 3310 | 626 |

TABLE ATI

Probe Name Ag6413

| | | | | |
|---|---|---|---|---|
| Primers | Sequences | Length | Start Position | SEQ ID No |

| | | | | |
|---|---|---|---|---|
| Forward | 5′-ggtgaagacaagatctgccag-3′ | 21 | 1968 | 627 |
| Probe | TET-5′-tgtacccgggtcagcgacacg-3′-TAMRA | 21 | 2019 | 628 |
| Reverse | 5′-gctgttgttccatccacatc-3′ | 20 | 2061 | 629 |

TABLE ATK

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatgca-3′ | 15 | 2974 | 630 |
| Probe | TET-5′-actctacagctttgaccgcgcgg-3′-TAMRA | 23 | 2945 | 631 |
| Reverse | 5′-gccaactgtgtggtgttca-3′ | 19 | 2919 | 632 |

TABLE ATL
CNS_neurodegeneration_v1.0

| | Rel. | Rel. | Rel. | Rel. | Rel. | Rel. | Rel. | Rel. |
|---|---|---|---|---|---|---|---|---|
| | Exp. (%) | Exp. (%) | Exp. (%) | Exp. (%) | Exp. (%) | Exp. (%) | Exp. (%) | Exp. (%) |
| | Ag4983, | Ag6413, | Ag6428, | Ag6430, | Ag6431, | Ag6440, | Ag6442, | Ag6446, |
| Tissue | Run | Run | Run | Run | Run | Run | Run | Run |
| Name | 218649223 | 269253983 | 266937081 | 266937085 | 268030722 | 269254003 | 264979298 | 269254006 |
| AD 1 | 23.7 | 24.8 | 18.0 | 20.0 | 18.8 | 18.9 | 19.2 | 42.9 |
| Hippo | | | | | | | | |
| AD 2 | 41.2 | 52.9 | 32.3 | 48.0 | 28.7 | 61.1 | 49.7 | 41.8 |
| Hippo | | | | | | | | |
| AD 3 | 8.9 | 6.4 | 3.7 | 11.6 | 7.5 | 9.7 | 20.4 | 23.7 |
| Hippo | | | | | | | | |
| AD 4 | 14.8 | 25.5 | 10.7 | 17.1 | 18.8 | 23.3 | 5.6 | 29.9 |
| Hippo | | | | | | | | |
| AD 5 | 44.8 | 41.8 | 53.2 | 39.2 | 38.4 | 34.6 | 57.4 | 67.8 |
| Hippo | | | | | | | | |
| AD 6 | 100.0 | 100.0 | 100.0 | 100.0 | 100.0 | 100.0 | 90.1 | 100.0 |
| Hippo | | | | | | | | |
| Control 2 | 24.3 | 36.1 | 18.7 | 17.9 | 29.5 | 29.9 | 28.5 | 39.2 |
| Hippo | | | | | | | | |
| Control 4 | 42.9 | 43.8 | 27.0 | 38.4 | 32.3 | 54.7 | 86.5 | 62.4 |
| Hippo | | | | | | | | |
| Control | 14.2 | 11.4 | 4.6 | 10.2 | 6.0 | 5.8 | 0.0 | 14.6 |
| (Path) 3 | | | | | | | | |
| Hippo | | | | | | | | |
| AD 1 | 23.3 | 15.9 | 12.9 | 12.1 | 17.1 | 12.6 | 16.8 | 72.7 |
| Temporal | | | | | | | | |
| Ctx | | | | | | | | |
| AD 2 | 41.5 | 47.3 | 31.0 | 36.6 | 39.8 | 59.0 | 21.6 | 43.2 |
| Temporal | | | | | | | | |
| Ctx | | | | | | | | |
| AD 3 | 9.5 | 9.8 | 6.0 | 11.7 | 11.3 | 17.1 | 5.7 | 36.3 |
| Temporal | | | | | | | | |
| Ctx | | | | | | | | |
| AD 4 | 30.6 | 39.0 | 20.2 | 15.6 | 25.3 | 29.9 | 8.7 | 43.2 |
| Temporal | | | | | | | | |
| Ctx | | | | | | | | |
| AD 5 Inf | 45.4 | 37.1 | 39.2 | 43.8 | 36.3 | 41.8 | 73.7 | 63.3 |
| Temporal | | | | | | | | |
| Ctx | | | | | | | | |
| AD 5 Sup | 51.1 | 39.0 | 42.0 | 56.6 | 32.3 | 39.2 | 55.9 | 95.3 |
| Temporal | | | | | | | | |
| Ctx | | | | | | | | |
| AD 6 Inf | 38.2 | 59.9 | 49.3 | 40.9 | 46.7 | 48.6 | 76.8 | 45.1 |
| Temporal | | | | | | | | |
| Ctx | | | | | | | | |
| AD 6 Sup | 43.8 | 48.6 | 48.3 | 44.1 | 50.3 | 17.0 | 59.9 | 30.6 |
| Temporal | | | | | | | | |
| Ctx | | | | | | | | |
| Control 1 | 12.2 | 23.0 | 12.9 | 11.9 | 15.6 | 23.3 | 46.7 | 5.9 |
| Temporal | | | | | | | | |
| Ctx | | | | | | | | |
| Control 2 | 14.2 | 32.5 | 18.2 | 16.7 | 17.4 | 43.5 | 50.0 | 13.6 |
| Temporal | | | | | | | | |
| Ctx | | | | | | | | |
| Control 3 | 15.1 | 15.3 | 9.6 | 13.0 | 14.5 | 9.2 | 9.5 | 12.5 |
| Temporal | | | | | | | | |
| Ctx | | | | | | | | |
| Control 3 | 23.7 | 25.0 | 15.2 | 18.9 | 13.1 | 30.1 | 13.6 | 26.6 |
| Temporal | | | | | | | | |
| Ctx | | | | | | | | |
| Control | 26.1 | 47.0 | 27.0 | 32.5 | 30.6 | 51.1 | 46.0 | 21.2 |
| (Path) 1 | | | | | | | | |
| Temporal | | | | | | | | |
| Ctx | | | | | | | | |
| Control | 24.5 | 25.9 | 16.0 | 19.5 | 20.4 | 7.2 | 0.0 | 27.2 |
| (Path) 2 | | | | | | | | |
| Temporal | | | | | | | | |
| Ctx | | | | | | | | |
| Control | 11.7 | 16.0 | 7.5 | 12.9 | 10.9 | 9.9 | 31.0 | 24.5 |
| (Path) 3 | | | | | | | | |
| Temporal | | | | | | | | |
| Ctx | | | | | | | | |
| Control | 21.9 | 27.4 | 17.1 | 19.8 | 18.2 | 14.9 | 39.5 | 19.2 |
| (Path) 4 | | | | | | | | |
| Temporal | | | | | | | | |
| Ctx | | | | | | | | |
| AD 1 | 16.0 | 11.9 | 10.2 | 16.2 | 11.5 | 5.8 | 6.3 | 39.5 |
| Occipital | | | | | | | | |
| Ctx | | | | | | | | |
| AD 2 | 0.0 | 0.0 | 0.0 | 0.0 | 0.0 | 0.0 | 0.0 | 0.0 |
| Occipital | | | | | | | | |
| Ctx | | | | | | | | |
| (Missing) | | | | | | | | |
| AD 3 | 10.7 | 6.0 | 6.4 | 11.7 | 8.8 | 7.8 | 4.9 | 19.3 |
| Occipital | | | | | | | | |
| Ctx | | | | | | | | |
| AD 4 | 18.9 | 23.7 | 13.0 | 12.6 | 7.9 | 35.4 | 11.1 | 25.3 |
| Occipital | | | | | | | | |
| Ctx | | | | | | | | |
| AD 5 | 24.8 | 28.3 | 25.3 | 16.7 | 22.5 | 16.6 | 42.3 | 25.2 |
| Occipital | | | | | | | | |
| Ctx | | | | | | | | |
| AD 6 | 20.6 | 31.9 | 20.2 | 17.8 | 17.0 | 23.5 | 14.8 | 9.7 |
| Occipital | | | | | | | | |
| Ctx | | | | | | | | |
| Control 1 | 9.5 | 14.4 | 6.0 | 11.3 | 8.7 | 15.2 | 8.8 | 6.5 |
| Occipital | | | | | | | | |
| Ctx | | | | | | | | |
| Control 2 | 31.9 | 42.6 | 26.4 | 24.8 | 33.2 | 35.8 | 82.4 | 8.1 |
| Occipital | | | | | | | | |
| Ctx | | | | | | | | |
| Control 3 | 18.8 | 13.0 | 10.7 | 16.4 | 17.1 | 4.4 | 8.8 | 15.8 |
| Occipital | | | | | | | | |
| Ctx | | | | | | | | |
| Control 4 | 18.2 | 17.0 | 12.0 | 12.1 | 12.6 | 12.9 | 24.0 | 23.3 |
| Occipital | | | | | | | | |
| Ctx | | | | | | | | |
| Control | 38.2 | 52.5 | 35.6 | 32.8 | 36.1 | 22.4 | 100.0 | 23.3 |
| (Path) 1 | | | | | | | | |
| Occipital | | | | | | | | |
| Ctx | | | | | | | | |
| Control | 9.6 | 14.1 | 6.7 | 9.6 | 7.9 | 5.0 | 9.3 | 15.6 |
| (Path) 2 | | | | | | | | |
| Occipital | | | | | | | | |
| Ctx | | | | | | | | |
| Control | 4.8 | 8.7 | 5.4 | 8.4 | 6.0 | 6.7 | 4.1 | 4.5 |
| (Path) 3 | | | | | | | | |
| Occipital | | | | | | | | |
| Ctx | | | | | | | | |
| Control | 16.2 | 13.2 | 13.2 | 15.9 | 10.2 | 11.9 | 32.8 | 5.9 |
| (Path) 4 | | | | | | | | |
| Occipital | | | | | | | | |
| Ctx | | | | | | | | |
| Control 1 | 14.4 | 21.9 | 8.8 | 15.2 | 16.3 | 33.2 | 9.2 | 5.7 |
| Parietal Ctx | | | | | | | | |
| Control 2 | 32.8 | 28.9 | 34.4 | 39.5 | 28.3 | 17.4 | 28.1 | 74.2 |
| Parietal Ctx | | | | | | | | |
| Control 3 | 20.6 | 19.8 | 11.5 | 14.5 | 8.7 | 21.6 | 9.1 | 8.6 |
| Parietal Ctx | | | | | | | | |
| Control | 35.4 | 62.4 | 34.2 | 33.4 | 39.2 | 47.3 | 69.3 | 24.0 |
| (Path) 1 | | | | | | | | |
| Parietal Ctx | | | | | | | | |
| Control | 22.1 | 23.8 | 19.6 | 20.0 | 22.5 | 17.1 | 37.6 | 23.7 |
| (Path) 2 | | | | | | | | |
| Parietal Ctx | | | | | | | | |
| Control | 11.2 | 15.4 | 3.9 | 15.0 | 7.1 | 11.7 | 10.4 | 11.0 |
| (Path) 3 | | | | | | | | |
| Parietal Ctx | | | | | | | | |
| Control | 31.2 | 34.2 | 24.8 | 28.3 | 8.8 | 29.3 | 27.5 | 27.0 |
| (Path) 4 | | | | | | | | |
| Parietal Ctx | | | | | | | | |

TABLE ATM
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 13.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro; met) SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE ATN
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro; met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE ATO
General_screening_panel_v1.6

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag6413, | Ag6424, | Ag6425, | Ag6428, | Ag6430, |
| Tissue | Run | Run | Run | Run | Run |
| Name | 277249371 | 277221719 | 277221721 | 277222439 | 277222443 |
| Adipose | 25.9 | 0.0 | 2.6 | 20.0 | 8.2 |
| Melanoma* | 0.5 | 0.0 | 0.0 | 2.0 | 0.5 |
| Hs688(A).T | | | | | |
| Melanoma* | 2.7 | 0.0 | 0.2 | 4.1 | 0.6 |
| Hs688(B).T | | | | | |
| Melanoma* | 0.3 | 0.0 | 0.0 | 0.7 | 0.7 |
| M14 | | | | | |
| Melanoma* | 0.0 | 0.0 | 0.0 | 0.1 | 0.0 |
| LOXIMVI | | | | | |
| Melanoma* | 15.2 | 0.0 | 2.2 | 30.4 | 22.5 |
| SK-MEL-5 | | | | | |
| Squamous | 0.0 | 0.0 | 0.0 | 0.1 | 0.3 |
| cell | | | | | |
| carcinoma | | | | | |
| SCC-4 | | | | | |
| Testis Pool | 5.2 | 0.0 | 3.5 | 8.8 | 4.2 |
| Prostate | 1.9 | 0.0 | 0.5 | 2.5 | 1.0 |
| ca.* (bone | | | | | |
| met) PC-3 | | | | | |
| Prostate | 8.1 | 0.0 | 1.0 | 11.5 | 8.5 |
| Pool | | | | | |
| Placenta | 0.5 | 0.0 | 0.0 | 0.7 | 0.1 |
| Uterus Pool | 2.2 | 0.0 | 1.5 | 4.5 | 2.6 |
| Ovarian ca. | 0.9 | 0.0 | 0.3 | 1.1 | 0.8 |
| OVCAR-3 | | | | | |
| Ovarian ca. | 0.8 | 0.0 | 0.2 | 1.7 | 1.5 |
| SK-OV-3 | | | | | |
| Ovarian ca. | 0.2 | 0.0 | 0.0 | 0.9 | 0.5 |
| OVCAR-4 | | | | | |
| Ovarian ca. | 1.6 | 0.0 | 1.3 | 2.9 | 1.5 |
| OVCAR-5 | | | | | |
| Ovarian ca. | 100.0 | 100.0 | 100.0 | 77.9 | 90.8 |
| IGROV-1 | | | | | |
| Ovarian ca. | 13.6 | 5.6 | 21.9 | 14.0 | 11.9 |
| OVCAR-8 | | | | | |
| Ovary | 2.7 | 0.0 | 0.3 | 5.2 | 2.1 |
| Breast ca. | 0.3 | 0.0 | 0.0 | 0.3 | 0.4 |
| MCF-7 | | | | | |
| Breast ca. | 0.1 | 0.0 | 0.0 | 0.4 | 0.4 |
| MDA-MB- | | | | | |
| 231 | | | | | |
| Breast ca. | 0.5 | 0.0 | 0.0 | 0.5 | 0.3 |
| BT 549 | | | | | |
| Breast ca. | 0.0 | 0.0 | 0.0 | 0.5 | 0.3 |
| T47D | | | | | |
| Breast ca. | 0.6 | 0.0 | 0.0 | 0.7 | 0.7 |
| MDA-N | | | | | |
| Breast Pool | 15.0 | 0.0 | 4.1 | 21.8 | 19.5 |
| Trachea | 4.5 | 0.0 | 0.7 | 8.4 | 2.9 |
| Lung | 2.8 | 0.0 | 0.7 | 2.3 | 1.3 |
| Fetal Lung | 3.9 | 0.0 | 0.3 | 9.1 | 4.0 |
| Lung ca. | 2.0 | 2.0 | 0.9 | 3.5 | 2.7 |
| NCI-N417 | | | | | |
| Lung ca. | 3.5 | 3.1 | 2.7 | 6.5 | 7.0 |
| LX-1 | | | | | |
| Lung ca. | 0.1 | 0.0 | 0.0 | 0.3 | 0.5 |
| NCI-H146 | | | | | |
| Lung ca. | 4.0 | 2.3 | 0.4 | 6.8 | 6.3 |
| SHP-77 | | | | | |
| Lung ca. | 0.3 | 0.0 | 2.6 | 0.9 | 0.3 |
| A549 | | | | | |
| Lung ca. | 0.2 | 0.0 | 0.0 | 0.9 | 0.7 |
| NCI-H526 | | | | | |
| Lung ca. | 2.9 | 0.0 | 1.0 | 4.6 | 4.5 |
| NCI-H23 | | | | | |
| Lung ca. | 0.0 | 0.0 | 0.0 | 0.2 | 0.2 |
| NCI-H460 | | | | | |
| Lung ca. | 0.5 | 0.0 | 0.0 | 0.5 | 0.6 |
| HOP-62 | | | | | |
| Lung ca. | 1.7 | 0.0 | 0.6 | 2.3 | 2.4 |
| NCI-H522 | | | | | |
| Liver | 0.1 | 0.0 | 0.0 | 0.0 | 0.1 |
| Fetal Liver | 0.3 | 0.0 | 0.3 | 1.1 | 0.6 |
| Liver ca. | 0.1 | 0.0 | 0.3 | 0.2 | 0.1 |
| HepG2 | | | | | |
| Kidney | 27.9 | 6.5 | 0.0 | 47.0 | 34.9 |
| Pool | | | | | |
| Fetal | 1.4 | 0.0 | 0.0 | 4.9 | 5.1 |
| Kidney | | | | | |
| Renal ca. | 0.2 | 0.0 | 0.0 | 0.2 | 0.2 |
| 786-0 | | | | | |
| Renal ca. | 0.0 | 0.0 | 1.8 | 0.2 | 0.1 |
| A498 | | | | | |
| Renal ca. | 1.5 | 0.0 | 0.5 | 2.5 | 0.7 |
| ACHN | | | | | |
| Renal ca. | 0.3 | 0.0 | 0.0 | 0.5 | 0.3 |
| UO-31 | | | | | |
| Renal ca. | 1.9 | 0.0 | 0.4 | 3.1 | 2.5 |
| TK-10 | | | | | |
| Bladder | 4.2 | 0.0 | 0.0 | 5.9 | 3.0 |
| Gastric ca. | 0.9 | 0.0 | 0.0 | 1.7 | 1.7 |
| (liver met.) | | | | | |
| NCI-N87 | | | | | |
| Gastric ca. | 0.4 | 0.0 | 0.5 | 0.8 | 0.4 |
| KATO III | | | | | |
| Colon ca. | 0.0 | 0.0 | 1.5 | 0.2 | 0.0 |
| SW-948 | | | | | |
| Colon ca. | 20.9 | 9.5 | 5.2 | 41.8 | 39.0 |
| SW480 | | | | | |
| Colon ca.* | 13.3 | 7.7 | 4.8 | 16.4 | 15.5 |
| (SW480 met) | | | | | |
| SW620 | | | | | |
| Colon ca. | 0.2 | 0.0 | 0.0 | 0.0 | 0.0 |
| HT29 | | | | | |
| Colon ca. | 2.1 | 1.6 | 0.2 | 3.2 | 3.8 |
| HCT-116 | | | | | |
| Colon ca. | 15.0 | 10.4 | 3.6 | 27.0 | 22.2 |
| CaCo-2 | | | | | |
| Colon | 9.0 | 0.0 | 3.3 | 11.0 | 6.5 |
| cancer | | | | | |
| tissue | | | | | |
| Colon ca. | 1.3 | 0.0 | 3.0 | 2.5 | 1.7 |
| SW1116 | | | | | |
| Colon ca. | 0.1 | 0.0 | 0.4 | 0.3 | 0.2 |
| Colo-205 | | | | | |
| Colon ca. | 0.8 | 0.0 | 3.6 | 1.4 | 1.3 |
| SW-48 | | | | | |
| Colon Pool | 20.3 | 0.0 | 5.0 | 28.1 | 28.7 |
| Small | 14.0 | 0.0 | 1.7 | 17.1 | 10.5 |
| Intestine | | | | | |
| Pool | | | | | |
| Stomach | 8.1 | 0.0 | 2.3 | 14.3 | 6.2 |
| Pool | | | | | |
| Bone | 6.8 | 0.0 | 1.6 | 14.3 | 11.3 |
| Marrow | | | | | |
| Pool | | | | | |
| Fetal Heart | 10.1 | 0.0 | 2.3 | 25.5 | 24.3 |
| Heart Pool | 28.7 | 5.2 | 7.0 | 29.7 | 23.0 |
| Lymph | 17.6 | 0.0 | 6.1 | 33.7 | 30.4 |
| Node Pool | | | | | |
| Fetal | 31.9 | 36.9 | 5.2 | 54.3 | 46.7 |
| Skeletal | | | | | |
| Muscle | | | | | |
| Skeletal | 17.4 | 12.3 | 9.2 | 29.3 | 21.5 |
| Muscle | | | | | |
| Pool | | | | | |
| Spleen Pool | 0.9 | 0.0 | 0.0 | 1.9 | 2.0 |
| Thymus | 4.4 | 0.0 | 2.0 | 10.4 | 7.5 |
| Pool | | | | | |
| CNS cancer | 9.8 | 1.6 | 1.5 | 14.9 | 6.1 |
| (glio/astro) | | | | | |
| U87-MG | | | | | |
| CNS cancer | 3.5 | 0.0 | 0.3 | 4.7 | 2.9 |
| (glio/astro) | | | | | |
| U-118-MG | | | | | |
| CNS cancer | 1.9 | 0.0 | 0.0 | 2.6 | 1.7 |
| (neuro; met) | | | | | |
| SK-N-AS | | | | | |
| CNS cancer | 0.1 | 0.0 | 0.0 | 0.0 | 0.2 |
| (astro) SF- | | | | | |
| 539 | | | | | |
| CNS cancer | 8.1 | 1.9 | 1.1 | 14.9 | 5.9 |
| (astro) | | | | | |
| SNB-75 | | | | | |
| CNS cancer | 79.6 | 84.1 | 79.0 | 100.0 | 100.0 |
| (glio) SNB- | | | | | |
| 19 | | | | | |
| CNS cancer | 8.2 | 1.8 | 0.0 | 11.3 | 9.0 |
| (glio) SF- | | | | | |
| 295 | | | | | |
| Brain | 3.7 | 2.3 | 0.8 | 7.7 | 6.9 |
| (Amygdala) | | | | | |
| Pool | | | | | |
| Brain | 12.0 | 6.6 | 0.4 | 19.8 | 11.1 |
| (cere- | | | | | |
| bellum) | | | | | |
| Brain | 4.2 | 3.0 | 0.7 | 12.7 | 11.5 |
| (fetal) | | | | | |
| Brain | 7.5 | 3.1 | 3.2 | 11.7 | 11.0 |
| (Hippocam- | | | | | |
| pus) Pool | | | | | |
| Cerebral | 9.7 | 1.7 | 0.6 | 11.0 | 7.5 |
| Cortex Pool | | | | | |
| Brain | 7.4 | 1.8 | 2.2 | 11.7 | 8.5 |
| (Substantia | | | | | |
| nigra) Pool | | | | | |
| Brain | 7.6 | 0.0 | 2.7 | 13.2 | 10.0 |
| (Thalamus) | | | | | |
| Pool | | | | | |
| Brain | 6.1 | 0.0 | 0.4 | 10.6 | 8.0 |
| (whole) | | | | | |
| Spinal Cord | 10.1 | 3.2 | 2.3 | 14.7 | 12.8 |
| Pool | | | | | |
| Adrenal | 3.5 | 0.0 | 0.3 | 9.9 | 6.1 |
| Gland | | | | | |
| Pituitary | 0.9 | 0.0 | 0.0 | 1.1 | 0.8 |
| gland Pool | | | | | |
| Salivary | 0.9 | 0.0 | 0.0 | 1.8 | 1.1 |
| Gland | | | | | |
| Thyroid | 2.0 | 0.0 | 0.3 | 3.1 | 0.8 |
| (female) | | | | | |
| Pancreatic | 0.5 | 0.0 | 0.0 | 0.8 | 0.8 |
| ca. | | | | | |
| CAPAN2 | | | | | |
| Pancreas | 1.2 | 0.0 | 0.0 | 2.0 | 1.1 |
| Pool | | | | | |
| | Rel. | Rel. | Rel. | Rel. | Rel. |
| | Exp. (%) | Exp. (%) | Exp. (%) | Exp. (%) | Exp. (%) |
| | Ag6431, | Ag6431, | Ag6440, | Ag6446, | Ag6964, |
| Tissue | Run | Run | Run | Run | Run |
| Name | 277633568 | 278389390 | 277223177 | 277250179 | 278388946 |
| Adipose | 17.4 | 13.8 | 3.7 | 1.7 | 18.8 |
| Melanoma* | 0.8 | 0.9 | 0.0 | 0.1 | 0.7 |
| Hs688(A).T | | | | | |
| Melanoma* | 2.5 | 2.2 | 0.8 | 0.1 | 2.4 |
| Hs688(B).T | | | | | |
| Melanoma* | 0.4 | 0.4 | 0.0 | 0.1 | 0.7 |
| M14 | | | | | |
| Melanoma* | 0.0 | 0.0 | 0.0 | 0.1 | 0.1 |
| LOXIMVI | | | | | |
| Melanoma* | 18.2 | 14.6 | 3.0 | 6.8 | 15.9 |
| SK-MEL-5 | | | | | |
| Squamous | 0.1 | 0.2 | 0.0 | 0.0 | 0.1 |
| cell | | | | | |
| carcinoma | | | | | |
| SCC-4 | | | | | |
| Testis Pool | 10.4 | 9.0 | 3.0 | 5.8 | 9.9 |
| Prostate | 1.9 | 1.8 | 1.2 | 7.7 | 4.3 |
| ca.* (bone | | | | | |
| met) PC-3 | | | | | |
| Prostate | 11.3 | 12.1 | 2.1 | 1.9 | 10.0 |
| Pool | | | | | |
| Placenta | 0.1 | 0.1 | 0.0 | 0.9 | 0.4 |
| Uterus Pool | 4.6 | 4.5 | 2.3 | 0.3 | 4.1 |
| Ovarian ca. | 0.7 | 1.1 | 0.4 | 4.8 | 4.0 |
| OVCAR-3 | | | | | |
| Ovarian ca. | 0.8 | 0.9 | 0.5 | 2.5 | 1.7 |
| SK-OV-3 | | | | | |
| Ovarian ca. | 0.4 | 0.8 | 0.0 | 0.5 | 0.5 |
| OVCAR-4 | | | | | |
| Ovarian ca. | 1.3 | 1.7 | 4.2 | 15.6 | 7.9 |
| OVCAR-5 | | | | | |
| Ovarian ca. | 84.7 | 97.9 | 100.0 | 5.4 | 75.8 |
| IGROV-1 | | | | | |
| Ovarian ca. | 15.6 | 14.6 | 18.2 | 4.2 | 16.7 |
| OVCAR-8 | | | | | |
| Ovary | 3.1 | 2.3 | 0.8 | 0.2 | 2.4 |
| Breast ca. | 0.1 | 0.2 | 0.3 | 0.9 | 0.5 |
| MCF-7 | | | | | |
| Breast ca. | 0.2 | 0.2 | 0.0 | 0.2 | 0.3 |
| MDA-MB- | | | | | |
| 231 | | | | | |
| Breast ca. | 0.1 | 0.5 | 0.0 | 0.2 | 0.4 |
| BT 549 | | | | | |
| Breast ca. | 0.2 | 0.3 | 0.3 | 0.7 | 0.5 |
| T47D | | | | | |
| Breast ca. | 0.6 | 0.6 | 0.3 | 0.0 | 0.8 |
| MDA-N | | | | | |
| Breast Pool | 14.6 | 10.7 | 3.5 | 2.0 | 16.7 |
| Trachea | 4.8 | 4.2 | 1.4 | 0.5 | 5.6 |
| Lung | 4.2 | 3.2 | 5.3 | 0.5 | 5.1 |
| Fetal Lung | 5.0 | 4.8 | 2.9 | 0.5 | 6.1 |
| Lung ca. | 3.3 | 2.6 | 2.0 | 0.4 | 2.3 |
| NCI-N417 | | | | | |
| Lung ca. | 5.0 | 3.5 | 6.3 | 100.0 | 44.1 |
| LX-1 | | | | | |
| Lung ca. | 0.1 | 0.2 | 0.0 | 0.1 | 0.1 |
| NCI-H146 | | | | | |
| Lung ca. | 5.3 | 4.5 | 0.8 | 0.1 | 3.8 |
| SHP-77 | | | | | |
| Lung ca. | 0.0 | 0.4 | 2.2 | 14.3 | 4.7 |
| A549 | | | | | |
| Lung ca. | 0.6 | 0.3 | 0.3 | 0.0 | 0.5 |
| NCI-H526 | | | | | |
| Lung ca. | 4.8 | 3.2 | 2.3 | 15.9 | 10.3 |
| NCI-H23 | | | | | |
| Lung ca. | 0.1 | 0.3 | 0.0 | 0.1 | 0.3 |
| NCI-H460 | | | | | |
| Lung ca. | 1.0 | 0.6 | 0.0 | 0.2 | 0.7 |
| HOP-62 | | | | | |
| Lung ca. | 1.7 | 1.3 | 2.5 | 27.7 | 8.9 |
| NCI-H522 | | | | | |
| Liver | 0.0 | 0.0 | 0.4 | 5.3 | 2.0 |
| Fetal Liver | 0.6 | 0.5 | 0.8 | 23.0 | 8.2 |
| Liver ca. | 0.0 | 0.2 | 0.9 | 7.3 | 2.4 |
| HepG2 | | | | | |
| Kidney | 33.9 | 28.1 | 14.6 | 5.3 | 32.8 |
| Pool | | | | | |
| Fetal | 4.1 | 4.0 | 3.4 | 20.2 | 11.5 |
| Kidney | | | | | |
| Renal ca. | 0.3 | 0.1 | 0.0 | 1.7 | 0.9 |
| 786-0 | | | | | |
| Renal ca. | 0.0 | 0.3 | 3.8 | 23.0 | 8.5 |
| A498 | | | | | |
| Renal ca. | 1.7 | 1.5 | 0.5 | 3.8 | 2.5 |
| ACHN | | | | | |
| Renal ca. | 0.2 | 0.2 | 0.0 | 0.7 | 0.3 |
| UO-31 | | | | | |
| Renal ca. | 2.0 | 1.9 | 0.5 | 6.4 | 4.6 |
| TK-10 | | | | | |
| Bladder | 5.5 | 5.1 | 0.9 | 3.2 | 6.7 |
| Gastric ca. | 0.9 | 1.2 | 0.8 | 17.8 | 6.7 |
| (liver met.) | | | | | |
| NCI-N87 | | | | | |
| Gastric ca. | 0.2 | 0.3 | 0.4 | 1.3 | 0.9 |
| KATO III | | | | | |
| Colon ca. | 0.2 | 0.2 | 2.2 | 6.1 | 1.2 |
| SW-948 | | | | | |
| Colon ca. | 27.0 | 23.3 | 6.3 | 39.0 | 33.7 |
| SW480 | | | | | |
| Colon ca.* | 12.8 | 10.3 | 7.2 | 71.2 | 25.0 |
| (SW480 met) | | | | | |
| SW620 | | | | | |
| Colon ca. | 0.2 | 0.2 | 0.3 | 3.5 | 0.3 |
| HT29 | | | | | |
| Colon ca. | 2.5 | 2.0 | 0.6 | 6.4 | 4.3 |
| HCT-116 | | | | | |
| Colon ca. | 19.1 | 16.7 | 6.5 | 78.5 | 38.2 |
| CaCo-2 | | | | | |
| Colon | 11.9 | 7.6 | 4.4 | 21.9 | 20.4 |
| cancer | | | | | |
| tissue | | | | | |
| Colon ca. | 2.0 | 1.5 | 2.1 | 19.5 | 6.6 |
| SW1116 | | | | | |
| Colon ca. | 0.2 | 0.0 | 1.3 | 3.0 | 0.8 |
| Colo-205 | | | | | |
| Colon ca. | 1.5 | 1.5 | 3.0 | 4.2 | 2.6 |
| SW-48 | | | | | |
| Colon Pool | 23.2 | 18.7 | 8.1 | 3.1 | 20.6 |
| Small | 11.2 | 13.0 | 2.0 | 2.5 | 10.4 |
| Intestine | | | | | |
| Pool | | | | | |
| Stomach | 9.5 | 9.3 | 4.2 | 1.1 | 10.7 |
| Pool | | | | | |
| Bone | 10.2 | 8.7 | 3.5 | 1.1 | 12.5 |
| Marrow | | | | | |
| Pool | | | | | |
| Fetal Heart | 24.5 | 21.8 | 8.6 | 2.7 | 20.7 |
| Heart Pool | 25.9 | 17.2 | 10.7 | 3.4 | 26.1 |
| Lymph | 22.1 | 23.7 | 6.7 | 2.8 | 24.7 |
| Node Pool | | | | | |
| Fetal | 48.6 | 46.3 | 19.2 | 57.0 | 50.7 |
| Skeletal | | | | | |
| Muscle | | | | | |
| Skeletal | 29.5 | 25.9 | 22.7 | 24.3 | 32.3 |
| Muscle | | | | | |
| Pool | | | | | |
| Spleen Pool | 2.0 | 1.7 | 0.6 | 2.6 | 3.1 |
| Thymus | 8.1 | 9.4 | 3.1 | 1.4 | 7.0 |
| Pool | | | | | |
| CNS cancer | 10.7 | 10.0 | 2.2 | 6.3 | 14.1 |
| (glio/astro) | | | | | |
| U87-MG | | | | | |
| CNS cancer | 3.8 | 3.1 | 0.8 | 5.1 | 5.8 |
| (glio/astro) | | | | | |
| U-118-MG | | | | | |
| CNS cancer | 2.1 | 1.0 | 0.5 | 3.9 | 2.6 |
| (neuro; met) | | | | | |
| SK-N-AS | | | | | |
| CNS cancer | 0.1 | 0.2 | 0.2 | 0.3 | 0.1 |
| (astro) SF- | | | | | |
| 539 | | | | | |
| CNS cancer | 6.5 | 10.0 | 2.8 | 2.4 | 9.7 |
| (astro) | | | | | |
| SNB-75 | | | | | |
| CNS cancer | 100.0 | 100.0 | 97.9 | 5.2 | 100.0 |
| (glio) SNB- | | | | | |
| 19 | | | | | |
| CNS cancer | 8.0 | 7.8 | 1.5 | 14.9 | 14.8 |
| (glio) SF- | | | | | |
| 295 | | | | | |
| Brain | 6.2 | 4.8 | 4.4 | 1.1 | 5.3 |
| (Amygdala) | | | | | |
| Pool | | | | | |
| Brain | 10.7 | 9.7 | 1.2 | 1.4 | 9.7 |
| (cere- | | | | | |
| bellum) | | | | | |
| Brain | 6.6 | 5.6 | 2.1 | 1.1 | 6.4 |
| (fetal) | | | | | |
| Brain | 8.6 | 6.9 | 4.3 | 2.0 | 10.2 |
| (Hippocam- | | | | | |
| pus) Pool | | | | | |
| Cerebral | 7.5 | 0.7 | 2.0 | 2.0 | 8.7 |
| Cortex Pool | | | | | |
| Brain | 10.4 | 4.7 | 2.0 | 1.1 | 9.3 |
| (Substantia | | | | | |
| nigra) Pool | | | | | |
| Brain | 9.3 | 0.2 | 2.8 | 3.2 | 8.7 |
| (Thalamus) | | | | | |
| Pool | | | | | |
| Brain | 5.8 | 0.3 | 1.9 | 1.9 | 8.7 |
| (whole) | | | | | |
| Spinal Cord | 11.0 | 7.6 | 4.2 | 2.9 | 9.0 |
| Pool | | | | | |
| Adrenal | 3.9 | 3.7 | 0.9 | 0.7 | 4.1 |
| Gland | | | | | |
| Pituitary | 1.2 | 1.1 | 0.6 | 0.4 | 0.5 |
| gland Pool | | | | | |
| Salivary | 1.3 | 0.9 | 0.0 | 0.2 | 1.0 |
| Gland | | | | | |
| Thyroid | 2.5 | 2.5 | 1.3 | 0.8 | 2.3 |
| (female) | | | | | |
| Pancreatic | 0.7 | 0.6 | 0.6 | 4.6 | 2.2 |
| ca. | | | | | |
| CAPAN2 | | | | | |
| Pancreas | 1.1 | 1.6 | 1.0 | 2.6 | 2.3 |
| Pool | | | | | |

TABLE ATP
Panel 4.1D

| | Rel. | Rel. | Rel. | Rel. | Rel. | Rel. |
|---|---|---|---|---|---|---|
| | Exp. (%) | Exp. (%) | Exp. (%) | Exp. (%) | Exp. (%) | Exp. (%) |
| | Ag4983, | Ag6413, | Ag6425, | Ag6428, | Ag6430, | Ag6431, |
| | Run | Run | Run | Run | Run | Run |
| Tissue Name | 218623570 | 269239947 | 268713999 | 268767535 | 268767563 | 268767577 |
| Secondary Th1 act | 0.1 | 0.3 | 0.0 | 1.3 | 0.0 | 0.7 |
| Secondary Th2 act | 0.5 | 0.3 | 0.0 | 1.2 | 0.0 | 0.8 |
| Secondary Tr1 act | 0.0 | 0.0 | 0.0 | 0.0 | 0.0 | 0.7 |
| Secondary Th1 rest | 0.1 | 0.0 | 0.0 | 0.0 | 0.0 | 0.0 |
| Secondary Th2 rest | 0.3 | 0.0 | 0.0 | 0.0 | 0.0 | 0.0 |
| Secondary Tr1 rest | 0.1 | 0.3 | 0.0 | 0.4 | 0.0 | 0.0 |
| Primary Th1 act | 0.0 | 0.0 | 0.0 | 0.0 | 0.0 | 0.0 |
| Primary Th2 act | 0.2 | 0.4 | 0.0 | 0.3 | 0.0 | 0.4 |
| Primary Tr1 act | 0.1 | 0.0 | 0.0 | 0.7 | 0.0 | 0.7 |
| Primary Th1 rest | 0.0 | 0.0 | 0.0 | 0.1 | 0.0 | 0.3 |
| Primary Th2 rest | 0.0 | 0.0 | 0.0 | 0.4 | 0.0 | 0.2 |
| Primary Tr1 rest | 0.3 | 0.0 | 0.0 | 0.0 | 0.0 | 0.0 |
| CD45RA CD4 | 0.4 | 2.8 | 0.0 | 5.4 | 0.0 | 2.4 |
| lymphocyte act | | | | | | |
| CD45RO CD4 | 0.1 | 2.2 | 0.0 | 1.5 | 0.0 | 0.7 |
| lymphocyte act | | | | | | |
| CD8 lymphocyte act | 0.4 | 0.9 | 0.0 | 0.7 | 0.0 | 0.0 |
| Secondary CD8 | 0.1 | 0.0 | 0.0 | 8.8 | 0.0 | 0.0 |
| lymphocyte rest | | | | | | |
| Secondary CD8 | 0.0 | 0.1 | 0.0 | 0.4 | 0.0 | 0.3 |
| lymphocyte act | | | | | | |
| CD4 lymphocyte | 0.1 | 0.0 | 0.0 | 0.5 | 0.0 | 0.4 |
| none | | | | | | |
| 2ry | 0.3 | 0.2 | 0.0 | 0.0 | 0.0 | 0.0 |
| Th1/Th2/Tr1_anti- | | | | | | |
| CD95 CH11 | | | | | | |
| LAK cells rest | 5.6 | 5.0 | 2.7 | 11.8 | 0.1 | 3.8 |
| LAK ceils IL-2 | 0.4 | 0.3 | 0.0 | 0.0 | 0.0 | 0.0 |
| LAK cells IL-2 + | 0.2 | 0.0 | 0.0 | 0.0 | 0.0 | 0.0 |
| IL-12 | | | | | | |
| LAK cells IL-2 + | 0.1 | 0.3 | 0.0 | 0.0 | 0.0 | 0.0 |
| IFN gamma | | | | | | |
| LAK cells IL-2 + | 0.0 | 0.0 | 0.0 | 0.0 | 0.0 | 0.0 |
| IL-18 | | | | | | |
| LAK cells | 4.5 | 4.0 | 15.7 | 15.1 | 0.1 | 6.3 |
| PMA/ionomycin | | | | | | |
| NK Cells IL-2 rest | 0.9 | 0.1 | 0.0 | 3.4 | 0.0 | 2.5 |
| Two Way MLR 3 | 1.4 | 1.1 | 0.0 | 2.2 | 0.0 | 1.3 |
| day | | | | | | |
| Two Way MLR 5 | 4.5 | 0.9 | 0.0 | 0.8 | 0.0 | 0.9 |
| day | | | | | | |
| Two Way MLR 7 | 2.3 | 0.7 | 13.2 | 1.1 | 0.0 | 2.6 |
| day | | | | | | |
| PBMC rest | 0.1 | 0.0 | 0.0 | 0.0 | 0.0 | 0.0 |
| PBMC PWM | 0.6 | 0.0 | 0.0 | 1.3 | 0.0 | 0.0 |
| PBMC PHA-L | 0.3 | 0.2 | 0.0 | 0.6 | 0.0 | 0.7 |
| Ramos (B cell) none | 0.1 | 0.0 | 0.0 | 0.0 | 0.0 | 0.0 |
| Ramos (B cell) | 0.0 | 0.0 | 0.0 | 0.7 | 0.0 | 0.2 |
| ionomycin | | | | | | |
| B lymphocytes | 0.5 | 0.0 | 0.0 | 0.0 | 0.0 | 0.0 |
| PWM | | | | | | |
| B lymphocytes | 0.2 | 0.0 | 0.0 | 0.9 | 0.0 | 0.0 |
| CD40L and IL-4 | | | | | | |
| EOL-1 dbcAMP | 3.7 | 2.6 | 9.1 | 29.1 | 0.1 | 8.1 |
| EOL-1 dbcAMP | 1.6 | 0.7 | 0.0 | 0.0 | 0.0 | 2.7 |
| PMA/ionomycin | | | | | | |
| Dendritic cells none | 5.6 | 3.1 | 13.8 | 4.1 | 0.0 | 5.3 |
| Dendritic cells LPS | 1.6 | 0.3 | 0.0 | 1.0 | 0.0 | 0.7 |
| Dendritic cells anti- | 2.0 | 1.6 | 3.3 | 0.5 | 0.0 | 0.2 |
| CD40 | | | | | | |
| Monocytes rest | 0.2 | 0.0 | 0.0 | 0.4 | 0.0 | 0.0 |
| Monocytes LPS | 2.2 | 3.3 | 0.0 | 5.7 | 0.0 | 1.8 |
| Macrophages rest | 0.9 | 1.8 | 0.0 | 0.6 | 0.0 | 0.6 |
| Macrophages LPS | 7.5 | 4.0 | 0.0 | 5.4 | 0.1 | 6.3 |
| HUVEC none | 0.1 | 0.0 | 0.0 | 0.0 | 0.0 | 0.0 |
| HUVEC starved | 0.0 | 0.0 | 0.0 | 0.0 | 0.0 | 0.3 |
| HUVEC IL-1beta | 0.0 | 0.0 | 0.0 | 0.0 | 0.0 | 0.5 |
| HUVEC IFN | 0.2 | 0.0 | 0.0 | 0.0 | 0.0 | 0.0 |
| gamma | | | | | | |
| HUVEC TNF | 0.0 | 0.0 | 0.0 | 0.0 | 0.0 | 0.0 |
| alpha + | | | | | | |
| IFN gamma | | | | | | |
| HUVEC TNF | 0.6 | 0.0 | 0.0 | 0.0 | 0.0 | 0.4 |
| alpha + IL4 | | | | | | |
| HUVEC IL-11 | 0.0 | 0.0 | 0.0 | 0.4 | 0.0 | 0.3 |
| Lung Microvascular | 0.2 | 0.3 | 0.0 | 0.4 | 0.0 | 0.0 |
| EC none | | | | | | |
| Lung Microvascular | 0.1 | 0.0 | 0.0 | 0.0 | 0.0 | 0.0 |
| EC TNFalpha + IL- | | | | | | |
| 1beta | | | | | | |
| Microvascular | 0.1 | 0.0 | 0.0 | 0.0 | 0.0 | 0.0 |
| Dermal EC none | | | | | | |
| Microsvasular | 0.1 | 0.0 | 0.0 | 0.0 | 0.0 | 0.0 |
| Dermal EC | | | | | | |
| TNFalpha + IL- | | | | | | |
| 1beta | | | | | | |
| Bronchial | 0.0 | 0.0 | 0.0 | 0.0 | 0.0 | 0.0 |
| epithelium | | | | | | |
| TNFalpha + | | | | | | |
| IL1beta | | | | | | |
| Small airway | 0.0 | 0.0 | 0.0 | 0.0 | 0.0 | 0.0 |
| epithelium none | | | | | | |
| Small airway | 0.3 | 0.0 | 0.0 | 0.0 | 0.0 | 0.0 |
| epithelium | | | | | | |
| TNFalpha + IL- | | | | | | |
| 1beta | | | | | | |
| Coronery artery | 0.1 | 0.6 | 0.0 | 0.0 | 0.0 | 0.0 |
| SMC rest | | | | | | |
| Coronery artery | 0.4 | 0.9 | 6.2 | 0.3 | 0.0 | 1.5 |
| SMC TNFalpha + | | | | | | |
| IL-1beta | | | | | | |
| Astrocytes rest | 67.8 | 97.3 | 100.0 | 100.0 | 12.0 | 100.0 |
| Astrocytes | 100.0 | 100.0 | 74.2 | 97.3 | 100.0 | 74.7 |
| TNFalpha + IL- | | | | | | |
| 1beta | | | | | | |
| KU-812 (Basophil) | 0.1 | 0.0 | 0.0 | 0.0 | 0.0 | 0.4 |
| rest | | | | | | |
| KU-812 (Basophil) | 0.0 | 0.0 | 0.0 | 0.0 | 0.0 | 0.0 |
| PMA/ionomycin | | | | | | |
| CCD1106 | 0.2 | 0.0 | 0.0 | 0.0 | 0.0 | 0.8 |
| (Keratinocytes) none | | | | | | |
| CCD1106 | 0.3 | 0.0 | 0.0 | 0.0 | 0.0 | 0.0 |
| (Keratinocytes) | | | | | | |
| TNFalpha + | | | | | | |
| IL-1beta | | | | | | |
| Liver cirrhosis | 2.3 | 7.2 | 4.6 | 2.6 | 0.0 | 6.7 |
| NCI-H292 none | 0.3 | 0.3 | 0.0 | 1.7 | 0.0 | 0.6 |
| NCI-H292 IL-4 | 0.3 | 0.0 | 0.0 | 0.0 | 0.0 | 0.5 |
| NCI-H292 IL-9 | 0.3 | 0.0 | 0.0 | 0.7 | 0.0 | 0.5 |
| NCI-H292 IL-13 | 0.6 | 0.6 | 0.0 | 0.9 | 0.0 | 0.9 |
| NCI-H292 IFN | 0.2 | 0.0 | 0.0 | 0.5 | 0.0 | 0.6 |
| gamma | | | | | | |
| HPAEC none | 0.0 | 0.3 | 0.0 | 0.0 | 0.0 | 0.0 |
| HPAEC TNF | 0.0 | 0.3 | 0.0 | 0.0 | 0.0 | 0.0 |
| alpha + | | | | | | |
| IL-1 beta | | | | | | |
| Lung fibroblast none | 29.7 | 62.9 | 31.4 | 95.9 | 0.2 | 65.5 |
| Lung fibroblast TNF | 16.0 | 36.9 | 22.2 | 48.6 | 0.1 | 39.8 |
| alpha + IL-1 beta | | | | | | |
| Lung fibroblast IL-4 | 26.1 | 28.7 | 19.1 | 27.4 | 0.1 | 21.2 |
| Lung fibroblast IL-9 | 28.5 | 42.0 | 23.5 | 24.0 | 0.1 | 26.8 |
| Lung fibroblast IL- | 31.6 | 14.6 | 4.5 | 11.9 | 0.0 | 10.4 |
| 13 | | | | | | |
| Lung fibroblast IFN | 20.4 | 32.8 | 15.7 | 55.9 | 0.2 | 46.3 |
| gamma | | | | | | |
| Dermal fibroblast | 2.5 | 2.9 | 0.0 | 6.0 | 0.0 | 6.3 |
| CCD1070 rest | | | | | | |
| Dermal fibroblast | 1.1 | 1.3 | 0.0 | 2.7 | 0.0 | 0.8 |
| CCD1070 TNF | | | | | | |
| alpha | | | | | | |
| Dermal fibroblast | 1.9 | 2.9 | 0.0 | 5.6 | 0.0 | 1.3 |
| CCD1070 IL-1 beta | | | | | | |
| Dermal fibroblast | 9.3 | 20.3 | 8.5 | 30.6 | 0.1 | 20.2 |
| IFN gamma | | | | | | |
| Dermal fibroblast | 10.7 | 14.6 | 4.1 | 30.8 | 0.1 | 19.8 |
| IL-4 | | | | | | |
| Dermal Fibroblasts | 24.8 | 42.3 | 8.0 | 54.3 | 0.1 | 46.7 |
| rest | | | | | | |
| Neutrophils | 0.7 | 0.0 | 0.0 | 0.9 | 0.0 | 0.4 |
| TNFa + LPS | | | | | | |
| Neutrophils rest | 0.1 | 0.0 | 0.0 | 0.0 | 0.0 | 0.3 |
| Colon | 7.9 | 4.7 | 4.0 | 4.6 | 0.0 | 9.5 |
| Lung | 2.2 | 1.2 | 0.0 | 2.8 | 0.0 | 4.6 |
| Thymus | 3.1 | 0.8 | 0.0 | 0.0 | 0.0 | 0.4 |
| Kidney | 4.2 | 4.4 | 4.9 | 7.8 | 0.1 | 9.7 |

TABLE ATQ
general oncology screening panel_v_2.4

| | Rel. Exp. (%) | Rel. Exp. (%) |
|---|---|---|
| | Ag4983, Run | Ag6442, Run |
| Tissue Name | 260281959 | 264979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell carcinoma 3 | 5.6 | 8.3 |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate | 9.2 | 7.5 |
| adenocarcinoma 1 | | |
| Prostate | 3.5 | 8.0 |
| adenocarcinoma 2 | | |
| Prostate | 14.3 | 9.0 |
| adenocarcinoma 3 | | |
| Prostate | 16.4 | 9.1 |
| adenocarcinoma 4 | | |
| Prostate NAT 5 | 16.8 | 9.9 |
| Prostate | 3.2 | 7.7 |
| adenocarcinoma 6 | | |
| Prostate | 9.2 | 17.3 |
| adenocarcinoma 7 | | |
| Prostate | 3.0 | 0.0 |
| adenocarcinoma 8 | | |
| Prostate | 27.0 | 33.9 |
| adenocarcinoma 9 | | |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0 Summary: Ag4983/Ag64113/Ag6428/Ag6430/Ag6431/Ag6440/Ag6442/Ag6446 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6424/Ag6425 Expression of this gene is low/undetectable (CTs>35) across all of the samples on this panel (data not shown).

General_screening_panel_v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening_panel_v1.6 Summary: Ag6413/Ag6424/Ag6425/Ag6428/Ag6431/Ag6440/Ag6446/Ag6964 Highest expression of this gene is detected in skeletal muscle, ovarian cancer IGROV-1 cell line, lung cancer LX-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Panel 4.1D Summary: Ag4983/Ag6413/Ag6425/Ag6428/Ag6431 Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

Ag6424/Ag6440 Expression of this gene is low/undetectable (CTs>35) across all of the samples on this panel (data not shown).

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AU. CG56054-14: Integrin Alpha 7-Like Protein.

Expression of gene CG56054-14 was assessed using the primer-probe sets Ag4983, Ag6442, Ag6428, Ag6429, Ag6431, Ag6435, Ag6439, Ag6447, Ag6413 and Ag6964, described in Tables AUA, AUB, AUC, AUD, AUE, AUF, AUG, AUH, AUI and AUJ. Results of the RTQ-PCR runs are shown in Tables AUK, AUL, AUM, AUN, AUO and AUP.

TABLE AUA

Probe Name Ag4983

| | | | Start | |
|---|---|---|---|---|
| Primers | Sequences | Length | Position | SEQ ID No |
| Forward | 5′-ccaggtcaccttctacctcatc-3′ | 22 | 2342 | 633 |
| Probe | TET-5′-cttagcacctccgggatcagcatt-3′-TAMRA | 24 | 2364 | 634 |
| Reverse | 5′-aacagcagctctacctccagtt-3′ | 22 | 2398 | 635 |

TABLE AUB

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3′ | 22 | 2781 | 636 |
| Probe | TET-5′-ccacctgagcagcagaggct-3′-TAMRA | 21 | 2820 | 637 |
| Reverse | 5′-gcgcagtccagggtg-3′ | 15 | 2906 | 638 |

TABLE AUC

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1301 | 639 |
| Probe | TET-5′-ccttcacaqgtgctggagggc-3′-TAMRA | 21 | 1341 | 640 |
| Reverse | 5′-agggagtagccgaagctct-3′ | 19 | 1378 | 641 |

TABLE AUD

Probe Name Ag6429

| | | | | |
|---|---|---|---|---|
| Primers | Sequences | Length | Start Position | SEQ ID No |

| | | | | |
|---|---|---|---|---|
| Forward | 5′-ccgtgccccagtaccat-3′ | 17 | 3289 | 642 |
| Probe | TET-5′-cgggcaccatcctgaggaacaac-3′-TAMRA | 23 | 3355 | 643 |
| Reverse | 5′-gggcccagccaggat-3′ | 15 | 3391 | 644 |

TABLE AUE

Probe Name Ag6431

| | | | Start | |
|---|---|---|---|---|
| Primers | Sequences | Length | Position | SEQ ID No |
| Forward | 5′-aaacatcaccctggactgc-3′ | 19 | 2900 | 645 |
| Probe | TET-5′-ggtgttcagctgcccactctacag-3′-TAMRA | 25 | 2941 | 646 |
| Reverse | 5′-ccgcgcggtcaaa-3′ | 13 | 2967 | 647 |

TABLE AUF

Probe Name Ag6435

| | | | | |
|---|---|---|---|---|
| Primers | Sequences | Length | Start Position | SEQ ID No |

| | | | | |
|---|---|---|---|---|
| Forward | 5′-ggccagggtggagct-3′ | 15 | 731 | 648 |
| Probe | TET-5′-acctggcacacctggacgacg-3′-TAMRA | 21 | 766 | 649 |
| Reverse | 5′-cagggaccgggatga-3′ | 15 | 829 | 650 |

TABLE AUG

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 3157 | 651 |
| Probe | TET-5′-ccctggtgggtcatcctcctg-3′-TAMRA | 21 | 3177 | 652 |
| Reverse | 5′-gaagaatcccatcttccacag-3′ | 21 | 3243 | 653 |

TABLE AUH

Probe Name Ag6447

| | | | Start | |
|---|---|---|---|---|
| Primers | Sequences | Length | Position | SEQ ID No |
| Forward | 5′-gacgacggtccctacga-3′ | 17 | 780 | 654 |
| Probe | TET-5′-tcatcccggtccctgccaa-3′-TAMRA | 19 | 829 | 655 |
| Reverse | 5′-gtcaatagagaagccaaagtagct-3′ | 24 | 849 | 656 |

TABLE AUI

Probe Name Ag6413

| | | | | |
|---|---|---|---|---|
| Primers | Sequences | Length | Start Position | SEQ ID No |

| | | | | |
|---|---|---|---|---|
| Forward | 5′-ggtgaagacaagatctgccag-3′ | 21 | 1980 | 657 |
| Probe | TET-5′-tgtacccgggtcagcgacacg-3′-TAMRA | 21 2031 | 658 | |
| Reverse | 5′-gctgttgttccatccacatc-3′ | 20 | 6073 | 659 |

TABLE AUJ

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatgca-3′ | 15 | 2986 | 660 |
| Probe | TET-5′-actctacagctttgaccgcgcgg-3′-TAMRA | 23 | 2957 | 661 |
| Reverse | 5′-gccaactgtgtggtgttaa-3′ | 19 | 2931 | 662 |

TABLE AUK
CNS_neurodegeneration_v1.0

| | Rel. Exp. | Rel. Exp. | Rel. Exp. | Rel. Exp. | Rel. Exp. | Rel. Exp. | Rel. Exp. | Rel. Exp. |
|---|---|---|---|---|---|---|---|---|
| | (%) Ag4983, | (%) Ag6413, | (%) Ag6428, | (%) Ag6431, | (%) Ag6435, | (%) Ag6439, | (%) Ag6442, | (%) Ag6447, |
| Tissue | Run | Run | Run | Run | Run | Run | Run | Run |
| Name | 218649223 | 269253983 | 266937081 | 268030722 | 269253997 | 269254002 | 264979298 | 269254007 |
| AD 1 | 23.7 | 24.8 | 18.0 | 18.8 | 17.1 | 21.6 | 19.2 | 18.8 |
| Hippo | | | | | | | | |
| AD 2 | 41.2 | 52.9 | 32.3 | 28.7 | 27.9 | 28.9 | 49.7 | 10.4 |
| Hippo | | | | | | | | |
| AD 3 | 8.9 | 6.4 | 3.7 | 7.5 | 4.8 | 6.1 | 20.4 | 0.0 |
| Hippo | | | | | | | | |
| AD 4 | 14.8 | 25.5 | 10.7 | 18.8 | 18.3 | 17.6 | 5.6 | 4.6 |
| Hippo | | | | | | | | |
| AD 5 | 44.8 | 41.8 | 53.2 | 38.4 | 46.7 | 42.6 | 57.4 | 11.0 |
| Hippo | | | | | | | | |
| AD 6 | 100.0 | 100.0 | 100.0 | 100.0 | 100.0 | 100.0 | 90.1 | 100.0 |
| Hippo | | | | | | | | |
| Control 2 | 24.3 | 36.1 | 18.7 | 29.5 | 8.5 | 32.5 | 28.5 | 3.1 |
| Hippo | | | | | | | | |
| Control 4 | 42.9 | 43.8 | 27.0 | 32.3 | 29.9 | 37.9 | 86.5 | 43.8 |
| Hippo | | | | | | | | |
| Control | 14.2 | 11.4 | 4.6 | 6.0 | 5.2 | 6.4 | 0.0 | 5.3 |
| (Path) 3 | | | | | | | | |
| Hippo | | | | | | | | |
| AD 1 | 23.3 | 15.9 | 12.9 | 17.1 | 12.8 | 24.5 | 16.8 | 9.0 |
| Temporal | | | | | | | | |
| Ctx | | | | | | | | |
| AD 2 | 41.5 | 47.3 | 31.0 | 39.8 | 45.1 | 27.5 | 21.6 | 21.0 |
| Temporal | | | | | | | | |
| Ctx | | | | | | | | |
| AD 3 | 9.5 | 9.8 | 6.0 | 11.3 | 4.1 | 9.0 | 5.7 | 3.9 |
| Temporal | | | | | | | | |
| Ctx | | | | | | | | |
| AD 4 | 30.6 | 39.0 | 20.2 | 25.3 | 6.8 | 30.4 | 8.7 | 7.7 |
| Temporal | | | | | | | | |
| Ctx | | | | | | | | |
| AD 5 Inf | 45.4 | 37.1 | 39.2 | 36.3 | 1.6 | 41.8 | 73.7 | 23.7 |
| Temporal | | | | | | | | |
| Ctx | | | | | | | | |
| AD 5 Sup | 51.1 | 39.0 | 42.0 | 32.3 | 33.2 | 38.7 | 55.9 | 11.4 |
| Temporal | | | | | | | | |
| Ctx | | | | | | | | |
| AD 6 Inf | 38.2 | 59.9 | 49.3 | 46.7 | 52.1 | 47.6 | 76.8 | 88.9 |
| Temporal | | | | | | | | |
| Ctx | | | | | | | | |
| AD 6 Sup | 43.8 | 48.6 | 48.3 | 50.3 | 37.6 | 50.3 | 59.9 | 61.1 |
| Temporal | | | | | | | | |
| Ctx | | | | | | | | |
| Control 1 | 12.2 | 23.0 | 12.9 | 15.6 | 6.7 | 24.0 | 46.7 | 2.8 |
| Temporal | | | | | | | | |
| Ctx | | | | | | | | |
| Control 2 | 14.2 | 32.5 | 18.2 | 17.4 | 7.3 | 14.9 | 50.0 | 16.0 |
| Temporal | | | | | | | | |
| Ctx | | | | | | | | |
| Control 3 | 15.1 | 15.3 | 9.6 | 14.5 | 4.4 | 16.5 | 9.5 | 3.1 |
| Temporal | | | | | | | | |
| Ctx | | | | | | | | |
| Control 3 | 23.7 | 25.0 | 15.2 | 13.1 | 11.7 | 23.8 | 13.6 | 13.6 |
| Temporal | | | | | | | | |
| Ctx | | | | | | | | |
| Control | 26.1 | 47.0 | 27.0 | 30.6 | 24.8 | 39.8 | 46.0 | 13.8 |
| (Path) 1 | | | | | | | | |
| Temporal | | | | | | | | |
| Ctx | | | | | | | | |
| Control | 24.5 | 25.9 | 16.0 | 20.4 | 9.8 | 24.8 | 0.0 | 2.6 |
| (Path) 2 | | | | | | | | |
| Temporal | | | | | | | | |
| Ctx | | | | | | | | |
| Control | 11.7 | 16.0 | 7.5 | 10.9 | 3.5 | 11.9 | 31.0 | 6.3 |
| (Path) 3 | | | | | | | | |
| Temporal | | | | | | | | |
| Ctx | | | | | | | | |
| Control | 21.9 | 27.4 | 17.1 | 18.2 | 14.8 | 21.6 | 39.5 | 7.0 |
| (Path) 4 | | | | | | | | |
| Temporal | | | | | | | | |
| Ctx | | | | | | | | |
| AD 1 | 16.0 | 11.9 | 10.2 | 11.5 | 15.0 | 16.0 | 6.3 | 0.0 |
| Occipital | | | | | | | | |
| Ctx | | | | | | | | |
| AD 2 | 0.0 | 0.0 | 0.0 | 0.0 | 0.0 | 0.0 | 0.0 | 0.0 |
| Occipital | | | | | | | | |
| Ctx | | | | | | | | |
| (Missing) | | | | | | | | |
| AD 3 | 10.7 | 6.0 | 6.4 | 8.8 | 8.0 | 10.2 | 4.9 | 0.0 |
| Occipital | | | | | | | | |
| Ctx | | | | | | | | |
| AD 4 | 18.9 | 23.7 | 13.0 | 17.9 | 6.8 | 18.6 | 11.1 | 3.5 |
| Occipital | | | | | | | | |
| Ctx | | | | | | | | |
| AD 5 | 24.8 | 28.3 | 25.3 | 22.5 | 12.7 | 22.7 | 42.3 | 3.8 |
| Occipital | | | | | | | | |
| Ctx | | | | | | | | |
| AD 6 | 20.6 | 31.9 | 20.2 | 17.0 | 5.9 | 22.1 | 14.8 | 8.5 |
| Occipital | | | | | | | | |
| Ctx | | | | | | | | |
| Control 1 | 9.5 | 14.4 | 6.0 | 8.7 | 4.1 | 7.2 | 8.8 | 1.3 |
| Occipital | | | | | | | | |
| Ctx | | | | | | | | |
| Control 2 | 31.9 | 42.6 | 26.4 | 33.2 | 20.3 | 29.3 | 82.4 | 13.7 |
| Occipital | | | | | | | | |
| Ctx | | | | | | | | |
| Control 3 | 18.8 | 13.0 | 10.7 | 17.1 | 7.5 | 19.2 | 8.8 | 5.0 |
| Occipital | | | | | | | | |
| Ctx | | | | | | | | |
| Control 4 | 18.2 | 17.0 | 12.0 | 12.6 | 3.3 | 13.6 | 24.0 | 1.3 |
| Occipital | | | | | | | | |
| Ctx | | | | | | | | |
| Control | 38.2 | 52.5 | 35.6 | 36.1 | 25.9 | 39.5 | 100.0 | 12.1 |
| (Path) 1 | | | | | | | | |
| Occipital | | | | | | | | |
| Ctx | | | | | | | | |
| Control | 9.6 | 14.1 | 6.7 | 7.9 | 7.4 | 7.0 | 9.3 | 13.2 |
| (Path) 2 | | | | | | | | |
| Occipital | | | | | | | | |
| Ctx | | | | | | | | |
| Control | 4.8 | 8.7 | 5.4 | 6.0 | 2.3 | 5.9 | 4.1 | 9.4 |
| (Path) 3 | | | | | | | | |
| Occipital | | | | | | | | |
| Ctx | | | | | | | | |
| Control | 16.2 | 13.2 | 13.2 | 10.2 | 21.0 | 11.4 | 32.8 | 20.4 |
| (Path) 4 | | | | | | | | |
| Occipital | | | | | | | | |
| CtX | | | | | | | | |
| Control 1 | 14.4 | 21.9 | 8.8 | 16.3 | 12.5 | 15.7 | 9.2 | 5.0 |
| Parietal | | | | | | | | |
| Ctx | | | | | | | | |
| Control 2 | 32.8 | 28.9 | 34.4 | 28.3 | 41.2 | 37.1 | 28.1 | 25.5 |
| Parietal | | | | | | | | |
| Ctx | | | | | | | | |
| Control 3 | 20.6 | 19.8 | 11.5 | 8.7 | 13.2 | 10.8 | 9.1 | 16.7 |
| Parietal | | | | | | | | |
| Ctx | | | | | | | | |
| Control | 35.4 | 62.4 | 34.2 | 39.2 | 22.5 | 37.9 | 69.3 | 4.2 |
| (Path) 1 | | | | | | | | |
| Parietal | | | | | | | | |
| Ctx | | | | | | | | |
| Control | 22.1 | 23.8 | 19.6 | 22.5 | 26.8 | 18.7 | 37.6 | 14.4 |
| (Path) 2 | | | | | | | | |
| Parietal | | | | | | | | |
| Ctx | | | | | | | | |
| Control | 11.2 | 15.4 | 3.9 | 7.1 | 7.5 | 12.0 | 10.4 | 5.9 |
| (Path) 3 | | | | | | | | |
| Parietal | | | | | | | | |
| Ctx | | | | | | | | |
| Control | 31.2 | 34.2 | 24.8 | 8.8 | 20.6 | 27.9 | 27.5 | 9.4 |
| (Path) 4 | | | | | | | | |
| Parietal | | | | | | | | |
| Ctx | | | | | | | | |

TABLE AUL
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 13.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro;met) SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE AUM
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5. 2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro;met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE AUN
General_screening_panel_v1.6

| | Rel. Exp. (%) | Rel. Exp. (%) | Rel. Exp. (%) | Rel. Exp. (%) | Rel. Exp. (%) | Rel. Exp. (%) | Rel. Exp. (%) |
|---|---|---|---|---|---|---|---|
| | Ag6413, | Ag6428, | Ag6431, | Ag6431, | Ag6435, | Ag6439, | Ag6964, |
| | Run | Run | Run | Run | Run | Run | Run |
| Tissue Name | 277249371 | 277222439 | 277633568 | 278389390 | 277223167 | 277223175 | 278388946 |
| Adipose | 25.9 | 20.0 | 17.4 | 13.8 | 13.2 | 17.3 | 18.8 |
| Melanoma* | 0.5 | 2.0 | 0.8 | 0.9 | 0.9 | 0.4 | 0.7 |
| Hs688(A).T | | | | | | | |
| Melanoma* | 2.7 | 4.1 | 2.5 | 2.2 | 1.9 | 2.9 | 2.4 |
| Hs688(B).T | | | | | | | |
| Melanoma* | 0.3 | 0.7 | 0.4 | 0.4 | 0.0 | 0.4 | 0.7 |
| M14 | | | | | | | |
| Melanoma* | 0.0 | 0.1 | 0.0 | 0.0 | 0.0 | 0.0 | 0.1 |
| LOXIMVI | | | | | | | |
| Melanoma* SK- | 15.2 | 30.4 | 18.2 | 14.6 | 4.4 | 18.3 | 15.9 |
| MEL-5 | | | | | | | |
| Squamous cell | 0.0 | 0.1 | 0.1 | 0.2 | 0.0 | 0.0 | 0.1 |
| carcinoma SCC- | | | | | | | |
| 4 | | | | | | | |
| Testis Pool | 5.2 | 8.8 | 10.4 | 9.0 | 10.0 | 9.1 | 9.9 |
| Prostate ca.* | 1.9 | 2.5 | 1.9 | 1.8 | 1.8 | 1.3 | 4.3 |
| (bone met) PC-3 | | | | | | | |
| Prostate Pool | 8.1 | 11.5 | 11.3 | 12.1 | 10.0 | 28.5 | 10.0 |
| Placenta | 0.5 | 0.7 | 0.1 | 0.1 | 0.3 | 0.5 | 0.4 |
| Uterus Pool | 2.2 | 4.5 | 4.6 | 4.5 | 16.2 | 5.3 | 4.1 |
| Ovarian ca. | 0.9 | 1.1 | 0.7 | 1.1 | 0.4 | 1.6 | 4.0 |
| OVCAR-3 | | | | | | | |
| Ovarian ca. SK- | 0.8 | 1.7 | 0.8 | 0.9 | 0.9 | 1.3 | 1.7 |
| OV-3 | | | | | | | |
| Ovarian ca. | 0.2 | 0.9 | 0.4 | 0.8 | 0.0 | 0.9 | 0.5 |
| OVCAR-4 | | | | | | | |
| Ovarian ca. | 1.6 | 2.9 | 1.3 | 1.7 | 0.3 | 1.4 | 7.9 |
| OVCAR-5 | | | | | | | |
| Ovarian ca. | 100.0 | 77.9 | 84.7 | 97.9 | 27.0 | 69.3 | 75.8 |
| IGROV-1 | | | | | | | |
| Ovarian ca. | 13.6 | 14.0 | 15.6 | 14.6 | 7.6 | 17.3 | 16.7 |
| OVCAR-8 | | | | | | | |
| Ovary | 2.7 | 5.2 | 3.1 | 2.3 | 4.5 | 2.8 | 2.4 |
| Breast ca. MCF-7 | 0.3 | 0.3 | 0.1 | 0.2 | 0.0 | 0.5 | 0.5 |
| Breast ca. | 0.1 | 0.4 | 0.2 | 0.2 | 0.0 | 0.2 | 0.3 |
| MDA-MB-231 | | | | | | | |
| Breast ca. BT | 0.5 | 0.5 | 0.1 | 0.5 | 0.0 | 0.6 | 0.4 |
| 549 | | | | | | | |
| Breast ca. T47D | 0.0 | 0.5 | 0.2 | 0.3 | 0.0 | 0.4 | 0.5 |
| Breast ca. | 0.6 | 0.7 | 0.6 | 0.6 | 0.7 | 0.6 | 0.8 |
| MDA-N | | | | | | | |
| Breast Pool | 15.0 | 21.8 | 14.6 | 10.7 | 42.9 | 12.2 | 16.7 |
| Trachea | 4.5 | 8.4 | 4.8 | 4.2 | 8.3 | 4.7 | 5.6 |
| Lung | 2.8 | 2.3 | 4.2 | 3.2 | 3.9 | 3.9 | 5.1 |
| Fetal Lung | 3.9 | 9.1 | 5.0 | 4.8 | 8.0 | 5.3 | 6.1 |
| Lung ca. NCI- | 2.0 | 3.5 | 3.3 | 2.6 | 0.2 | 4.0 | 2.3 |
| N417 | | | | | | | |
| Lung ca. LX-1 | 3.5 | 6.5 | 5.0 | 3.5 | 0.9 | 4.9 | 44.1 |
| Lung ca. NCI- | 0.1 | 0.3 | 0.1 | 0.2 | 0.0 | 0.1 | 0.1 |
| H146 | | | | | | | |
| Lung ca. SHP-77 | 4.0 | 6.8 | 5.3 | 4.5 | 0.2 | 4.5 | 3.8 |
| Lung ca. A549 | 0.3 | 0.9 | 0.0 | 0.4 | 0.0 | 0.6 | 4.7 |
| Lung ca. NCI- | 0.2 | 0.9 | 0.6 | 0.3 | 0.0 | 0.4 | 0.5 |
| H526 | | | | | | | |
| Lung ca. NCI- | 2.9 | 4.6 | 4.8 | 3.2 | 0.6 | 2.9 | 10.3 |
| H23 | | | | | | | |
| Lung ca. NCI- | 0.0 | 0.2 | 0.1 | 0.3 | 0.0 | 0.0 | 0.3 |
| H460 | | | | | | | |
| Lung ca. HOP- | 0.5 | 0.5 | 1.0 | 0.6 | 0.0 | 0.5 | 0.7 |
| 62 | | | | | | | |
| Lung ca. NCI- | 1.7 | 2.3 | 1.7 | 1.3 | 0.0 | 3.3 | 8.9 |
| H522 | | | | | | | |
| Liver | 0.1 | 0.0 | 0.0 | 0.0 | 0.0 | 0.1 | 2.0 |
| Fetal Liver | 0.3 | 1.1 | 0.6 | 0.5 | 0.3 | 0.8 | 8.2 |
| Liver ca. HepG2 | 0.1 | 0.2 | 0.0 | 0.2 | 0.0 | 0.1 | 2.4 |
| Kidney Pool | 27.9 | 47.0 | 33.9 | 28.1 | 100.0 | 43.2 | 32.8 |
| Fetal Kidney | 1.4 | 4.9 | 4.1 | 4.0 | 12.1 | 5.8 | 11.5 |
| Renal ca. 786-0 | 0.2 | 0.2 | 0.3 | 0.1 | 0.0 | 0.3 | 0.9 |
| Renal ca. A498 | 0.0 | 0.2 | 0.0 | 0.3 | 0.0 | 0.5 | 8.5 |
| Renal ca. | 1.5 | 2.5 | 1.7 | 1.5 | 0.0 | 1.2 | 2.5 |
| ACHN | | | | | | | |
| Renal ca. UO- | 0.3 | 0.5 | 0.2 | 0.2 | 0.0 | 0.6 | 0.3 |
| 31 | | | | | | | |
| Renal ca. TK-10 | 1.9 | 3.1 | 2.0 | 1.9 | 0.7 | 2.1 | 4.6 |
| Bladder | 4.2 | 5.9 | 5.5 | 5.1 | 6.6 | 8.3 | 6.7 |
| Gastric ca. (liver | 0.9 | 1.7 | 0.9 | 1.2 | 0.0 | 1.1 | 6.7 |
| met.) NCI-N87 | | | | | | | |
| Gastric ca. | 0.4 | 0.8 | 0.2 | 0.3 | 0.3 | 0.4 | 0.9 |
| KATO III | | | | | | | |
| Colon ca. SW- | 0.0 | 0.2 | 0.2 | 0.2 | 0.0 | 0.3 | 1.2 |
| 948 | | | | | | | |
| Colon ca. | 20.9 | 41.8 | 27.0 | 23.3 | 4.4 | 23.0 | 33.7 |
| SW480 | | | | | | | |
| Colon ca.* | 13.3 | 16.4 | 12.8 | 10.3 | 1.7 | 6.1 | 25.0 |
| (SW480 met) | | | | | | | |
| SW620 | | | | | | | |
| Colon ca. HT29 | 0.2 | 0.0 | 0.2 | 0.2 | 0.0 | 0.0 | 0.3 |
| Colon ca. HCT- | 2.1 | 3.2 | 2.5 | 2.0 | 0.5 | 2.1 | 4.3 |
| 116 | | | | | | | |
| Colon ca. CaCo-2 | 15.0 | 27.0 | 19.1 | 16.7 | 7.6 | 18.3 | 38.2 |
| Colon cancer | 9.0 | 11.0 | 11.9 | 7.6 | 5.6 | 7.7 | 20.4 |
| tissue | | | | | | | |
| Colon ca. | 1.3 | 2.5 | 2.0 | 1.5 | 1.1 | 1.8 | 6.0 |
| SW1116 | | | | | | | |
| Colon ca. Colo-205 | 0.1 | 0.3 | 0.2 | 0.0 | 0.0 | 0.2 | 0.8 |
| Colon ca. SW-48 | 0.8 | 1.4 | 1.5 | 1.5 | 0.0 | 1.4 | 2.6 |
| Colon Pool | 20.3 | 28.1 | 23.2 | 18.7 | 44.8 | 25.5 | 20.6 |
| Small Intestine | 14.0 | 17.1 | 11.2 | 13.0 | 26.8 | 12.8 | 10.4 |
| Pool | | | | | | | |
| Stomach Pool | 8.1 | 14.3 | 9.5 | 9.3 | 24.0 | 8.5 | 10.7 |
| Bone Marrow | 6.8 | 14.3 | 10.2 | 8.7 | 25.9 | 18.7 | 12.5 |
| Pool | | | | | | | |
| Fetal Heart | 10.1 | 25.5 | 24.5 | 21.8 | 31.6 | 33.7 | 20.7 |
| Heart Pool | 28.7 | 29.7 | 25.9 | 17.2 | 23.5 | 33.7 | 26.1 |
| Lymph Node | 17.6 | 33.7 | 22.1 | 23.7 | 64.6 | 19.9 | 24.7 |
| Pool | | | | | | | |
| Fetal Skeletal | 31.9 | 54.3 | 48.6 | 46.3 | 46.7 | 19.1 | 50.7 |
| Muscle | | | | | | | |
| Skeletal Muscle | 17.4 | 29.3 | 29.5 | 25.9 | 24.7 | 22.1 | 32.3 |
| Pool | | | | | | | |
| Spleen Pool | 0.9 | 1.9 | 2.0 | 1.7 | 2.4 | 2.7 | 3.1 |
| Thymus Pool | 4.4 | 10.4 | 8.1 | 9.4 | 18.4 | 7.7 | 7.0 |
| CNS cancer | 9.8 | 14.9 | 10.7 | 10.0 | 5.8 | 10.9 | 14.1 |
| (glio/astro) | | | | | | | |
| U87-MG | | | | | | | |
| CNS cancer | 3.5 | 4.7 | 3.8 | 3.1 | 1.5 | 3.8 | 5.8 |
| (glio/astro) U- | | | | | | | |
| 118-MG | | | | | | | |
| CNS cancer | 1.9 | 2.6 | 2.1 | 1.0 | 0.7 | 1.4 | 2.6 |
| (neuro; met) SK- | | | | | | | |
| N-AS | | | | | | | |
| CNS cancer | 0.1 | 0.0 | 0.1 | 0.2 | 0.2 | 0.1 | 0.1 |
| (astro) SF-539 | | | | | | | |
| CNS cancer | 8.1 | 14.9 | 6.5 | 10.0 | 3.1 | 11.7 | 9.7 |
| (astro) SNB-75 | | | | | | | |
| CNS cancer | 79.6 | 100.0 | 100.0 | 100.0 | 12.8 | 100.0 | 100.0 |
| (glio) SNB-19 | | | | | | | |
| CNS cancer | 8.2 | 11.3 | 8.0 | 7.8 | 0.0 | 8.2 | 14.8 |
| (glio) SF-295 | | | | | | | |
| Brain | 3.7 | 7.7 | 6.2 | 4.8 | 7.9 | 8.0 | 5.3 |
| (Amygdala) | | | | | | | |
| Pool | | | | | | | |
| Brain | 12.0 | 19.8 | 10.7 | 9.7 | 1.8 | 8.8 | 9.7 |
| (cerebellum) | | | | | | | |
| Brain (fetal) | 4.2 | 12.7 | 6.6 | 5.6 | 8.4 | 6.8 | 6.4 |
| Brain | 7.5 | 11.7 | 8.6 | 6.9 | 9.9 | 11.0 | 10.2 |
| (Hippocampus) | | | | | | | |
| Pool | | | | | | | |
| Cerebral Cortex | 9.7 | 11.0 | 7.5 | 0.7 | 1.8 | 11.6 | 8.7 |
| Pool | | | | | | | |
| Brain | 7.4 | 11.7 | 10.4 | 4.7 | 4.2 | 10.0 | 9.3 |
| (Substantia | | | | | | | |
| nigra) Pool | | | | | | | |
| Brain | 7.6 | 13.2 | 9.3 | 0.2 | 9.1 | 9.7 | 8.7 |
| (Thalamus) Pool | | | | | | | |
| Brain (whole) | 6.1 | 10.6 | 5.8 | 0.3 | 3.3 | 5.6 | 8.7 |
| Spinal Cord | 10.1 | 14.7 | 11.0 | 7.6 | 13.1 | 12.2 | 9.0 |
| Pool | | | | | | | |
| Adrenal Gland | 3.5 | 9.9 | 3.9 | 3.7 | 7.4 | 4.8 | 4.1 |
| Pituitary gland | 0.9 | 1.1 | 1.2 | 1.1 | 1.8 | 1.4 | 0.5 |
| Pool | | | | | | | |
| Salivary Gland | 0.9 | 1.8 | 1.3 | 0.9 | 2.3 | 1.1 | 1.0 |
| Thyroid | 2.0 | 3.1 | 2.5 | 2.5 | 3.3 | 1.9 | 2.3 |
| (female) | | | | | | | |
| Pancreatic ca. | 0.5 | 0.8 | 0.7 | 0.6 | 0.5 | 0.7 | 2.2 |
| CAPAN2 | | | | | | | |
| Pancreas Pool | 1.2 | 2.0 | 1.1 | 1.6 | 3.5 | 3.2 | 2.3 |

TABLE AUO
Panel 4.1D

| | Rel. Exp. (%) | Rel. Exp. (%) | Rel. Exp. (%) | Rel. Exp. (%) | Rel. Exp. (%) | Rel. Exp. (%) | Rel. Exp. (%) |
|---|---|---|---|---|---|---|---|
| | Ag4983, | Ag6413, | Ag6428, | Ag6431, | Ag6435, | Ag6439, | Ag6447, |
| | Run | Run | Run | Run | Run | Run | Run |
| Tissue Name | 218623570 | 269239947 | 268767535 | 268767577 | 268713480 | 268760823 | 268761806 |
| Secondary Th1 | 0.1 | 0.3 | 1.3 | 0.7 | 0.0 | 0.0 | 0.0 |
| act | | | | | | | |
| Secondary Th2 | 0.5 | 0.3 | 1.2 | 0.8 | 0.0 | 0.0 | 0.0 |
| act | | | | | | | |
| Secondary Tr1 | 0.0 | 0.0 | 0.0 | 0.7 | 0.0 | 0.0 | 0.0 |
| act | | | | | | | |
| Secondary Th1 | 0.1 | 0.0 | 0.0 | 0.0 | 0.0 | 0.0 | 0.0 |
| rest | | | | | | | |
| Secondary Th2 | 0.3 | 0.0 | 0.0 | 0.0 | 0.7 | 0.0 | 0.0 |
| rest | | | | | | | |
| Secondary Tr1 | 0.1 | 0.3 | 0.4 | 0.0 | 0.0 | 0.0 | 0.0 |
| rest | | | | | | | |
| Primary Th1 act | 0.0 | 0.0 | 0.0 | 0.0 | 0.0 | 0.0 | 0.0 |
| Primary Th2 act | 0.2 | 0.4 | 0.3 | 0.4 | 0.7 | 0.0 | 0.0 |
| Primary Tr1 act | 0.1 | 0.0 | 0.7 | 0.7 | 0.0 | 0.0 | 0.0 |
| Primary Th1 rest | 0.0 | 0.0 | 0.1 | 0.3 | 0.0 | 1.2 | 0.0 |
| Primary Th2 rest | 0.0 | 0.0 | 0.4 | 0.2 | 0.0 | 0.0 | 0.0 |
| Primary Tr1 rest | 0.3 | 0.0 | 0.0 | 0.0 | 0.0 | 0.0 | 0.0 |
| CD45RA CD4 | 0.4 | 2.8 | 5.4 | 2.4 | 0.8 | 2.6 | 0.0 |
| lymphocyte act | | | | | | | |
| CD45RO CD4 | 0.1 | 2.2 | 1.5 | 0.7 | 1.6 | 2.3 | 0.0 |
| lymphocyte act | | | | | | | |
| CD8 lymphocyte | 0.4 | 0.9 | 0.7 | 0.0 | 0.0 | 0.0 | 0.0 |
| act | | | | | | | |
| Secondary CD8 | 0.1 | 0.0 | 8.8 | 0.0 | 0.0 | 0.0 | 0.0 |
| lymphocyte rest | | | | | | | |
| Secondary CD8 | 0.0 | 0.1 | 0.4 | 0.3 | 0.0 | 0.0 | 0.0 |
| lymphocyte act | | | | | | | |
| CD4 lymphocyte | 0.1 | 0.0 | 0.5 | 0.4 | 0.0 | 0.0 | 0.0 |
| none | | | | | | | |
| 2ry | 0.3 | 0.2 | 0.0 | 0.0 | 0.0 | 1.2 | 0.0 |
| Th1/Th2/Tr1_{—} | | | | | | | |
| anti-CD95 CH11 | | | | | | | |
| LAK cells rest | 5.6 | 5.0 | 11.8 | 3.8 | 6.1 | 15.2 | 0.0 |
| LAK cells IL-2 | 0.4 | 0.3 | 0.0 | 0.0 | 0.0 | 0.0 | 0.0 |
| LAK cells IL- | 0.2 | 0.0 | 0.0 | 0.0 | 0.0 | 0.0 | 0.0 |
| 2 + IL-12 | | | | | | | |
| LAK cells IL- | 0.1 | 0.3 | 0.0 | 0.0 | 0.0 | 0.0 | 0.0 |
| 2 + IFN gamma | | | | | | | |
| LAK cells IL-2 + | 0.0 | 0.0 | 0.0 | 0.0 | 0.0 | 0.0 | 0.0 |
| L-18 | | | | | | | |
| LAK cells | 4.5 | 4.0 | 15.1 | 6.3 | 6.1 | 9.0 | 0.0 |
| PMA/ionomycin | | | | | | | |
| NK Cells IL-2 | 0.9 | 0.1 | 3.4 | 2.5 | 0.0 | 1.4 | 0.0 |
| rest | | | | | | | |
| Two Way MLR 3 | 1.4 | 1.1 | 2.2 | 1.3 | 0.9 | 1.4 | 0.0 |
| day | | | | | | | |
| Two Way MLR 5 | 4.5 | 0.9 | 0.8 | 0.9 | 0.0 | 0.0 | 0.0 |
| day | | | | | | | |
| Two Way MLR 7 | 2.3 | 0.7 | 1.1 | 2.6 | 2.9 | 3.7 | 0.0 |
| day | | | | | | | |
| PBMC rest | 0.1 | 0.0 | 0.0 | 0.0 | 0.0 | 0.0 | 0.0 |
| PBMC PWM | 0.6 | 0.0 | 1.3 | 0.0 | 0.0 | 0.0 | 0.0 |
| PBMC PHA-L | 0.3 | 0.2 | 0.6 | 0.7 | 0.0 | 0.0 | 0.0 |
| Ramos (B cell) | 0.1 | 0.0 | 0.0 | 0.0 | 0.0 | 0.0 | 0.0 |
| none | | | | | | | |
| Ramos (B cell) | 0.0 | 0.0 | 0.7 | 0.2 | 0.0 | 0.0 | 0.0 |
| ionomycin | | | | | | | |
| B lymphocytes | 0.5 | 0.0 | 0.0 | 0.0 | 0.0 | 0.0 | 0.0 |
| PWM | | | | | | | |
| B lymphocytes | 0.2 | 0.0 | 0.9 | 0.0 | 0.0 | 0.0 | 0.0 |
| CD40L and IL-4 | | | | | | | |
| EOL-1 dbcAMP | 3.7 | 2.6 | 29.1 | 8.1 | 0.0 | 68.8 | 0.0 |
| EOL-1 dbcAMP | 1.6 | 0.7 | 0.0 | 2.7 | 1.0 | 1.8 | 0.0 |
| PMA/ionomycin | | | | | | | |
| Dendritic cells | 5.6 | 3.1 | 4.1 | 5.3 | 0.7 | 0.0 | 0.0 |
| none | | | | | | | |
| Dendritic cells | 1.6 | 0.3 | 1.0 | 0.7 | 0.0 | 0.0 | 0.0 |
| LPS | | | | | | | |
| Dendritic cells | 2.0 | 1.6 | 0.5 | 0.2 | 1.6 | 0.0 | 0.0 |
| anti-CD40 | | | | | | | |
| Monocytes rest | 0.2 | 0.0 | 0.4 | 0.0 | 0.0 | 0.0 | 0.0 |
| Monocytes LPS | 2.2 | 3.3 | 5.7 | 1.8 | 0.0 | 2.6 | 0.4 |
| Macrophages rest | 0.9 | 1.8 | 0.6 | 0.6 | 0.0 | 0.0 | 0.0 |
| Macrophages LPS | 7.5 | 4.0 | 5.4 | 6.3 | 0.8 | 9.2 | 0.0 |
| HUVEC none | 0.1 | 0.0 | 0.0 | 0.0 | 0.0 | 0.0 | 0.0 |
| HUVEC starved | 0.0 | 0.0 | 0.0 | 0.3 | 0.0 | 0.0 | 0.0 |
| HUVEC IL-1beta | 0.0 | 0.0 | 0.0 | 0.5 | 0.0 | 0.0 | 0.0 |
| HUVEC IFN | 0.2 | 0.0 | 0.0 | 0.0 | 0.0 | 0.0 | 0.0 |
| gamma | | | | | | | |
| HUVEC TNF | 0.0 | 0.0 | 0.0 | 0.0 | 0.6 | 0.0 | 0.0 |
| alpha + IFN | | | | | | | |
| gamma | | | | | | | |
| HUVEC TNF | 0.6 | 0.0 | 0.0 | 0.4 | 0.0 | 0.0 | 0.0 |
| alpha + IL4 | | | | | | | |
| HUVEC IL-11 | 0.0 | 0.0 | 0.4 | 0.3 | 0.0 | 0.0 | 0.0 |
| Lung | 0.2 | 0.3 | 0.4 | 0.0 | 0.0 | 0.0 | 0.0 |
| Microvascular EC | | | | | | | |
| none | | | | | | | |
| Lung | 0.1 | 0.0 | 0.0 | 0.0 | 0.0 | 0.0 | 0.0 |
| Microvascular EC | | | | | | | |
| TNFalpha + IL- | | | | | | | |
| 1beta | | | | | | | |
| Microvascular | 0.1 | 0.0 | 0.0 | 0.0 | 0.0 | 0.0 | 0.0 |
| Dermal EC none | | | | | | | |
| Microsvasular | 0.1 | 0.0 | 0.0 | 0.0 | 0.0 | 0.0 | 0.0 |
| Dermal EC | | | | | | | |
| TNFalpha + IL- | | | | | | | |
| 1beta | | | | | | | |
| Bronchial | 0.0 | 0.0 | 0.0 | 0.0 | 0.0 | 0.0 | 0.0 |
| epithelium | | | | | | | |
| TNFalpha + | | | | | | | |
| IL1beta | | | | | | | |
| Small airway | 0.0 | 0.0 | 0.0 | 0.0 | 0.0 | 0.0 | 0.0 |
| epithelium none | | | | | | | |
| Small airway | 0.3 | 0.0 | 0.0 | 0.0 | 0.0 | 0.0 | 0.0 |
| epithelium | | | | | | | |
| TNFalpha + IL- | | | | | | | |
| 1beta | | | | | | | |
| Coronery artery | 0.1 | 0.6 | 0.0 | 0.0 | 0.5 | 0.0 | 0.3 |
| SMC rest | | | | | | | |
| Coronery artery | 0.4 | 0.9 | 0.3 | 1.5 | 0.0 | 0.0 | 0.0 |
| SMC TNFalpha + | | | | | | | |
| IL-1beta | | | | | | | |
| Astrocytes rest | 67.8 | 97.3 | 100.0 | 100.0 | 100.0 | 100.0 | 54.3 |
| Astrocytes | 100.0 | 100.0 | 97.3 | 74.7 | 97.9 | 95.9 | 100.0 |
| TNFalpha + IL- | | | | | | | |
| 1beta | | | | | | | |
| KU-812 | 0.1 | 0.0 | 0.0 | 0.4 | 0.0 | 0.0 | 0.0 |
| (Basophil) rest | | | | | | | |
| KU-812 | 0.0 | 0.0 | 0.0 | 0.0 | 0.0 | 0.0 | 0.0 |
| (Basophil) | | | | | | | |
| PMA/ionomycin | | | | | | | |
| CCD1106 | 0.2 | 0.0 | 0.0 | 0.8 | 0.0 | 0.0 | 0.0 |
| (Keratinocytes) | | | | | | | |
| none | | | | | | | |
| CCD1106 | 0.3 | 0.0 | 0.0 | 0.0 | 0.0 | 0.0 | 0.0 |
| (Keratinocytes) | | | | | | | |
| TNFalpha + IL- | | | | | | | |
| 1beta | | | | | | | |
| Liver cirrhosis | 2.3 | 7.2 | 2.6 | 6.7 | 5.1 | 8.5 | 0.6 |
| NCI-H292 none | 0.3 | 0.3 | 1.7 | 0.6 | 0.0 | 0.0 | 0.0 |
| NCI-H292 IL-4 | 0.3 | 0.0 | 0.0 | 0.5 | 0.0 | 0.0 | 0.0 |
| NCI-H292 IL-9 | 0.3 | 0.0 | 0.7 | 0.5 | 0.0 | 0.0 | 0.0 |
| NCI-H292 IL-13 | 0.6 | 0.6 | 0.9 | 0.9 | 0.0 | 0.0 | 0.0 |
| NCI-H292 IFN | 0.2 | 0.0 | 0.5 | 0.6 | 0.0 | 0.0 | 0.0 |
| gamma | | | | | | | |
| HPAEC none | 0.0 | 0.3 | 0.0 | 0.0 | 0.0 | 0.0 | 0.0 |
| HPAEC TNF | 0.0 | 0.3 | 0.0 | 0.0 | 0.0 | 0.0 | 0.0 |
| alpha + IL-1beta | | | | | | | |
| Lung fibroblast | 29.7 | 62.9 | 95.9 | 65.5 | 62.9 | 94.0 | 26.2 |
| none | | | | | | | |
| Lung fibroblast | 16.0 | 36.9 | 48.6 | 39.8 | 25.2 | 62.9 | 28.3 |
| TNF alpha + IL-1 | | | | | | | |
| beta | | | | | | | |
| Lung fibroblast | 26.1 | 28.7 | 27.4 | 21.2 | 23.3 | 34.9 | 16.0 |
| IL-4 | | | | | | | |
| Lung fibroblast | 28.5 | 42.0 | 24.0 | 26.8 | 20.4 | 96.6 | 9.3 |
| IL-9 | | | | | | | |
| Lung fibroblast | 31.6 | 14.6 | 11.9 | 10.4 | 15.0 | 13.4 | 4.3 |
| IL-13 | | | | | | | |
| Lung fibroblast | 20.4 | 32.8 | 55.9 | 46.3 | 29.9 | 89.5 | 25.2 |
| IFN gamma | | | | | | | |
| Dermal fibroblast | 2.5 | 2.9 | 6.0 | 6.3 | 5.6 | 4.1 | 0.0 |
| CCD1070 rest | | | | | | | |
| Dermal fibroblast | 1.1 | 1.3 | 2.7 | 0.8 | 0.8 | 2.3 | 1.1 |
| CCD1070 TNF | | | | | | | |
| alpha | | | | | | | |
| Dermal fibroblast | 1.9 | 2.9 | 5.6 | 1.3 | 0.7 | 0.0 | 1.6 |
| CCD1070 IL-1 | | | | | | | |
| beta | | | | | | | |
| Dermal fibroblast IFN gamma | 9.3 | 20.3 | 30.6 | 20.2 | 20.0 | 26.6 | 4.9 |
| Dermal fibroblast IL-4 | 10.7 | 14.6 | 30.8 | 19.8 | 22.7 | 25.5 | 13.5 |
| Dermal | 24.8 | 42.3 | 54.3 | 46.7 | 20.7 | 47.3 | 15.8 |
| Fibroblasts rest | | | | | | | |
| Neutrophils | 0.7 | 0.0 | 0.9 | 0.4 | 1.2 | 0.0 | 0.0 |
| TNFa + LPS | | | | | | | |
| Neutrophils rest | 0.1 | 0.0 | 0.0 | 0.3 | 0.0 | 0.0 | 0.0 |
| Colon | 7.9 | 4.7 | 4.6 | 9.5 | 7.9 | 8.4 | 4.8 |
| Lung | 2.2 | 1.2 | 2.8 | 4.6 | 1.6 | 2.1 | 0.0 |
| Thymus | 3.1 | 0.8 | 0.0 | 0.4 | 2.0 | 2.4 | 0.0 |
| Kidney | 4.2 | 4.4 | 7.8 | 9.7 | 10.2 | 5.2 | 0.6 |

TABLE AUP
general oncology screening panel_v_2.4

| | Rel. Exp. (%) | Rel. Exp. (%) |
|---|---|---|
| | Ag4983, Run | Ag6442, Run |
| Tissue Name | 260281959 | 64979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell | 5.6 | 8.3 |
| carcinoma 3 | | |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate | 9.2 | 7.5 |
| adenocarcinoma 1 | | |
| Prostate | 3.5 | 8.0 |
| adenocarcinoma 2 | | |
| Prostate | 14.3 | 9.0 |
| adenocarcinoma 3 | | |
| Prostate | 16.4 | 9.1 |
| adenocarcinoma 4 | | |
| Prostate NAT 5 | 16.8 | 9.9 |
| Prostate | 3.2 | 7.7 |
| adenocarcinoma 6 | | |
| Prostate | 9.2 | 17.3 |
| adenocarcinoma 7 | | |
| Prostate | 3.0 | 0.0 |
| adenocarcinoma 8 | | |
| Prostate | 27.0 | 33.9 |
| adenocarcinoma 9 | | |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0 Summary: Ag4983/Ag6413/Ag6428/Ag6431/Ag6435/Ag6439/Ag6442/Ag6447 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6429 Expression of this gene is low/undetectable (CTs>35) across all of the samples on this panel (data not shown).

General_screening_panel_v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening_panel_v1.6 Summary: Ag6413/Ag6428/Ag6431/Ag6435/Ag6439 Six experiments with seven different probe and primer sets are in very good agreement. Highest expression of this gene is detected in a ovarian cancer IGROV-1 cell line and brain cancer SNB-19 cell lines (CTs=25-28.5). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6429/Ag6447 Expression of this gene is low/undetectable (CTs>34.9) across all of the samples on this panel (data not shown).

Panel 4.1D

Summary: Ag4983/Ag6413/Ag6428/Ag6431/Ag6435/Ag6439/Ag6447 Seven experiments with different probe and primer sets are in excellent agreement. Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AV. CG56054-15: Integrin Alpha 7-Like Protein.

Expression of gene CG56054-15 was assessed using the primer-probe sets Ag6425, Ag6428, Ag6432, Ag6435 and Ag6447, described in Tables AVA, AVB, AVC, AVD and AVE. Results of the RTQ-PCR runs are shown in Tables AVF, AVG and AVH.

TABLE AVA

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 1888 | 663 |
| Probe | TET-5′-catcccgagctgggcccc-3′-TAMRA | 18 | 1920 | 664 |
| Reverse | 5′-gccctggatgcccat-3′ | 15 | 1939 | 665 |

TABLE AVB

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1301 | 666 |
| Probe | TET-5′-ccttcacaggtgctggagggc-3′-TAMRA | 21 | 1341 | 667 |
| Reverse | 5′-agggagtagccgaagctct-3′ | 19 | 1378 | 668 |

TABLE AVC

Probe Name Ag6432

| | | | Start | |
|---|---|---|---|---|
| Primers | Sequences | Length | Position | SEQ ID No |
| Forward | 5′-gaccttgtcctacagtctccagac-3′ | 24 | 1841 | 669 |
| Probe | TET-5′-tgcacaccccatcctggctgct-3′-TAMRA | 22 | 1892 | 670 |
| Reverse | 5′-gctcgggatgcccgt-3′ | 15 | 1915 | 671 |

TABLE AVD

Probe Name Ag6435

| | | | | |
|---|---|---|---|---|
| Primers | Sequences | Length | Start Position | SEQ ID No |

| | | | | |
|---|---|---|---|---|
| Forward | 5′-ggccagggtggagct-3′ | 15 | 731 | 672 |
| Probe | TET-5′-acctggcacacctggacgacg-3′-TAMRA | 21 | 766 | 673 |
| Reverse | 5′-cagggaccgggatga-3′ | 15 | 829 | 674 |

TABLE AVE

Probe Name Ag6447

| | | | Start | |
|---|---|---|---|---|
| Primers | Sequences | Length | Position | SEQ ID No |
| Forward | 5′-gacgacggtccctacga-3′ | 17 | 780 | 675 |
| Probe | TET-5′-tcatcccggtccctgccaa-3′-TAMRA | 19 | 829 | 676 |
| Reverse | 5′-gtcaatagagaagccaaagtagct-3′ | 24 | 849 | 677 |

TABLE AVF
CNS_neurodegeneration_v1.0

| | Rel. Exp. | Rel. Exp. | Rel. Exp. |
|---|---|---|---|
| | (%) Ag6428, | (%) Ag6435, | (%) Ag6447, |
| | Run | Run | Run |
| Tissue Name | 266937081 | 269253997 | 269254007 |
| AD 1 Hippo | 18.0 | 17.1 | 18.8 |
| AD 2 Hippo | 32.3 | 27.9 | 10.4 |
| AD 3 Hippo | 3.7 | 4.8 | 0.0 |
| AD 4 Hippo | 10.7 | 18.3 | 4.6 |
| AD 5 Hippo | 53.2 | 46.7 | 11.0 |
| AD 6 Hippo | 100.0 | 100.0 | 100.0 |
| Control 2 Hippo | 18.7 | 8.5 | 3.1 |
| Control 4 Hippo | 27.0 | 29.9 | 43.8 |
| Control (Path) 3 Hippo | 4.6 | 5.2 | 5.3 |
| AD 1 Temporal Ctx | 12.9 | 12.8 | 9.0 |
| AD 2 Temporal Ctx | 31.0 | 45.1 | 21.0 |
| AD 3 Temporal Ctx | 6.0 | 4.1 | 3.9 |
| AD 4 Temporal Ctx | 20.2 | 6.8 | 7.7 |
| AD 5 Inf Temporal Ctx | 39.2 | 1.6 | 23.7 |
| AD 5 Sup Temporal Ctx | 42.0 | 33.2 | 11.4 |
| AD 6 Inf Temporal Ctx | 49.3 | 52.1 | 88.9 |
| AD 6 Sup Temporal Ctx | 48.3 | 37.6 | 61.1 |
| Control 1 Temporal Ctx | 12.9 | 6.7 | 2.8 |
| Control 2 Temporal Ctx | 18.2 | 7.3 | 16.0 |
| Control 3 Temporal Ctx | 9.6 | 4.4 | 3.1 |
| Control 3 Temporal Ctx | 15.2 | 11.7 | 13.6 |
| Control (Path) 1 | 27.0 | 24.8 | 13.8 |
| Temporal Ctx | | | |
| Control (Path) 2 | 16.0 | 9.8 | 2.6 |
| Temporal Ctx | | | |
| Control (Path) 3 | 7.5 | 3.5 | 6.3 |
| Temporal Ctx | | | |
| Control (Path) 4 | 17.1 | 14.8 | 7.0 |
| Temporal Ctx | | | |
| AD 1 Occipital Ctx | 10.2 | 15.0 | 0.0 |
| AD 2 Occipital Ctx | 0.0 | 0.0 | 0.0 |
| (Missing) | | | |
| AD 3 Occipital Ctx | 6.4 | 8.0 | 0.0 |
| AD 4 Occipital Ctx | 13.0 | 6.8 | 3.5 |
| Ad 5 Occipital Ctx | 25.3 | 12.7 | 3.8 |
| AD 6 Occipital Ctx | 20.2 | 5.9 | 8.5 |
| Control 1 Occipital Ctx | 6.0 | 4.1 | 1.3 |
| Control 2 Occipital Ctx | 26.4 | 20.3 | 13.7 |
| Control 3 Occipital Ctx | 10.7 | 7.5 | 5.0 |
| Control 4 Occipital Ctx | 12.0 | 3.3 | 1.3 |
| Control (Path) 1 | 35.6 | 25.9 | 12.1 |
| Occipital Ctx | | | |
| Control (Path) 2 | 6.7 | 7.4 | 13.2 |
| Occipital Ctx | | | |
| Control (Path) 3 | 5.4 | 2.3 | 9.4 |
| Occipital Ctx | | | |
| Control (Path) 4 | 13.2 | 21.0 | 20.4 |
| Occipital Ctx | | | |
| Control 1 Parietal Ctx | 8.8 | 12.5 | 5.0 |
| Control 2 Parietal Ctx | 34.4 | 41.2 | 25.5 |
| Control 3 Parietal Ctx | 11.5 | 13.2 | 16.7 |
| Control (Path) 1 | 34.2 | 22.5 | 4.2 |
| Parietal Ctx | | | |
| Control (Path) 2 | 19.6 | 26.8 | 14.4 |
| Parietal Ctx | | | |
| Control (Path) 3 | 3.9 | 7.5 | 5.9 |
| Parietal Ctx | | | |
| Control (Path) 4 | 24.8 | 20.6 | 9.4 |
| Parietal Ctx | | | |

TABLE AVG
General_screening_panel_v1.6

| | Rel. Exp. (%) | Rel. Exp. (%) | Rel. Exp. (%) |
|---|---|---|---|
| | Ag6425, | Ag6428, | Ag6435, |
| | Run | Run | Run |
| Tissue Name | 277221721 | 277222439 | 277223167 |
| Adipose | 2.6 | 20.0 | 13.2 |
| Melanoma* | 0.0 | 2.0 | 0.9 |
| Hs688(A).T | | | |
| Melanoma* | 0.2 | 4.1 | 1.9 |
| Hs688(B).T | | | |
| Melanoma* | 0.0 | 0.7 | 0.0 |
| M14 | | | |
| Melanoma* | 0.0 | 0.1 | 0.0 |
| LOXIMVI | | | |
| Melanoma* SK- | 2.2 | 30.4 | 4.4 |
| MEL-5 | | | |
| Squamous cell | 0.0 | 0.1 | 0.0 |
| carcinoma SCC- | | | |
| 4 | | | |
| Testis Pool | 3.5 | 8.8 | 10.0 |
| Prostate ca.* | 0.5 | 2.5 | 1.8 |
| (bone met) PC-3 | | | |
| Prostate Pool | 1.0 | 11.5 | 10.0 |
| Placenta | 0.0 | 0.7 | 0.3 |
| Uterus Pool | 1.5 | 4.5 | 16.2 |
| Ovarian ca. | 0.3 | 1.1 | 0.4 |
| OVCAR-3 | | | |
| Ovarian ca. SK- | 0.2 | 1.7 | 0.9 |
| OV-3 | | | |
| Ovarian ca. | 0.0 | 0.9 | 0.0 |
| OVCAR-4 | | | |
| Ovarian ca. | 1.3 | 2.9 | 0.3 |
| OVCAR-5 | | | |
| Ovarian ca. | 100.0 | 77.9 | 27.0 |
| IGROV-1 | | | |
| Ovarian ca. | 21.9 | 14.0 | 7.6 |
| OVCAR-8 | | | |
| Ovary | 0.3 | 5.2 | 4.5 |
| Breast ca. MCF-7 | 0.0 | 0.3 | 0.0 |
| Breast ca. | 0.0 | 0.4 | 0.0 |
| MDA-MB-231 | | | |
| Breast ca. BT | 0.0 | 0.5 | 0.0 |
| 549 | | | |
| Breast ca. T47D | 0.0 | 0.5 | 0.0 |
| Breast ca. | 0.0 | 0.7 | 0.7 |
| MDA-N | | | |
| Breast Pool | 4.1 | 21.8 | 42.9 |
| Trachea | 0.7 | 8.4 | 8.3 |
| Lung | 0.7 | 2.3 | 3.9 |
| Fetal Lung | 0.3 | 9.1 | 8.0 |
| Lung ca. NCI- | 0.9 | 3.5 | 0.2 |
| N417 | | | |
| Lung ca. LX-1 | 2.7 | 6.5 | 0.9 |
| Lung ca. NCI- | 0.0 | 0.3 | 0.0 |
| H146 | | | |
| Lung ca. SHP-77 | 0.4 | 6.8 | 0.2 |
| Lung ca. A549 | 2.6 | 0.9 | 0.0 |
| Lung ca. NCI- | 0.0 | 0.9 | 0.0 |
| H526 | | | |
| Lung ca. NCI- | 1.0 | 4.6 | 0.6 |
| H23 | | | |
| Lung ca. NCI- | 0.0 | 0.2 | 0.0 |
| H460 | | | |
| Lung ca. HOP- | 0.0 | 0.5 | 0.0 |
| 62 | | | |
| Lung ca. NCI- | 0.6 | 2.3 | 0.0 |
| H522 | | | |
| Liver | 0.0 | 0.0 | 0.0 |
| Fetal Liver | 0.3 | 1.1 | 0.3 |
| Liver ca. | 0.3 | 0.2 | 0.0 |
| HepG2 | | | |
| Kidney Pool | 0.0 | 47.0 | 100.0 |
| Fetal Kidney | 0.0 | 4.9 | 12.1 |
| Renal ca. 786-0 | 0.0 | 0.2 | 0.0 |
| Renal ca. A498 | 1.8 | 0.2 | 0.0 |
| Renal ca. | 0.5 | 2.5 | 0.0 |
| ACHN | | | |
| Renal ca. UO-31 | 0.0 | 0.5 | 0.0 |
| Renal ca. TK- | 0.4 | 3.1 | 0.7 |
| 10 | | | |
| Bladder | 0.0 | 5.9 | 6.6 |
| Gastric ca. | 0.0 | 1.7 | 0.0 |
| (liver met.) | | | |
| NCI-N87 | | | |
| Gastric ca. | 0.5 | 0.8 | 0.3 |
| KATO III | | | |
| Colon ca. SW- | 1.5 | 0.2 | 0.0 |
| 948 | | | |
| Colon ca. | 5.2 | 41.8 | 4.4 |
| SW480 | | | |
| Colon ca.* | 4.8 | 16.4 | 1.7 |
| (SW480 met) | | | |
| SW620 | | | |
| Colon ca. | 0.0 | 0.0 | 0.0 |
| HT29 | | | |
| Colon ca. | 0.2 | 3.2 | 0.5 |
| HCT-116 | | | |
| Colon ca. | 3.6 | 27.0 | 7.6 |
| CaCo-2 | | | |
| Colon cancer | 3.3 | 11.0 | 5.6 |
| tissue | | | |
| Colon ca. | 3.0 | 2.5 | 1.1 |
| SW1116 | | | |
| Colon ca. | 0.4 | 0.3 | 0.0 |
| Colo-205 | | | |
| Colon ca. SW- | 3.6 | 1.4 | 0.0 |
| 48 | | | |
| Colon Pool | 5.0 | 28.1 | 44.8 |
| Small Intestine | 1.7 | 17.1 | 26.8 |
| Pool | | | |
| Stomach Pool | 2.3 | 14.3 | 24.0 |
| Bone Marrow | 1.6 | 14.3 | 25.9 |
| Pool | | | |
| Fetal Heart | 2.3 | 25.5 | 31.6 |
| Heart Pool | 7.0 | 29.7 | 23.5 |
| Lymph Node | 6.1 | 33.7 | 64.6 |
| Pool | | | |
| Fetal Skeletal | 5.2 | 54.3 | 46.7 |
| Muscle | | | |
| Skeletal | 9.2 | 29.3 | 24.7 |
| Muscle Pool | | | |
| Spleen Pool | 0.0 | 1.9 | 2.4 |
| Thymus Pool | 2.0 | 10.4 | 18.4 |
| CNS cancer | 1.5 | 14.9 | 5.8 |
| (glio/astro) | | | |
| U87-MG | | | |
| CNS cancer | 0.3 | 4.7 | 1.5 |
| (glio/astro) U- | | | |
| 118-MG | | | |
| CNS cancer | 0.0 | 2.6 | 0.7 |
| (neuro; met) | | | |
| SK-N-AS | | | |
| CNS cancer | 0.0 | 0.0 | 0.2 |
| (astro) SF-539 | | | |
| CNS cancer | 1.1 | 14.9 | 3.1 |
| (astro) SNB-75 | | | |
| CNS cancer | 79.0 | 100.0 | 12.8 |
| (glio) SNB-19 | | | |
| CNS cancer | 0.0 | 11.3 | 0.0 |
| (glio) SF-295 | | | |
| Brain | 0.8 | 7.7 | 7.9 |
| (Amygdala) | | | |
| Pool | | | |
| Brain | 0.4 | 19.8 | 1.8 |
| (cerebellum) | | | |
| Brain (fetal) | 0.7 | 12.7 | 8.4 |
| Brain | 3.2 | 11.7 | 9.9 |
| (Hippocampus) | | | |
| Pool | | | |
| Cerebral | 0.6 | 11.0 | 1.8 |
| Cortex Pool | | | |
| Brain | 2.2 | 11.7 | 4.2 |
| (Substantia | | | |
| nigra) Pool | | | |
| Brain | 2.7 | 13.2 | 9.1 |
| (Thalamus) | | | |
| Pool | | | |
| Brain (whole) | 0.4 | 10.6 | 3.3 |
| Spinal Cord | 2.3 | 14.7 | 13.1 |
| Pool | | | |
| Adrenal Gland | 0.3 | 9.9 | 7.4 |
| Pituitary gland | 0.0 | 1.1 | 1.8 |
| Pool | | | |
| Salivary Gland | 0.0 | 1.8 | 2.3 |
| Thyroid | 0.3 | 3.1 | 3.3 |
| (female) | | | |
| Pancreatic ca. | 0.0 | 0.8 | 0.5 |
| CAPAN2 | | | |
| Pancreas Pool | 0.0 | 2.0 | 3.5 |

TABLE AVH
Panel 4.1D

| | Rel. Exp. | Rel. Exp. | Rel. Exp. | Rel. Exp. |
|---|---|---|---|---|
| | (%) Ag6425, | (%) Ag6428, | (%) Ag6435, | (%) Ag6447, |
| | Run | Run | Run | Run |
| Tissue Name | 268713999 | 268767535 | 268713480 | 268761806 |
| Secondary Th1 act | 0.0 | 1.3 | 0.0 | 0.0 |
| Secondary Th2 act | 0.0 | 1.2 | 0.0 | 0.0 |
| Secondary Tr1 act | 0.0 | 0.0 | 0.0 | 0.0 |
| Secondary Th1 rest | 0.0 | 0.0 | 0.0 | 0.0 |
| Secondary Th2 rest | 0.0 | 0.0 | 0.7 | 0.0 |
| Secondary Tr1 rest | 0.0 | 0.4 | 0.0 | 0.0 |
| Primary Th1 act | 0.0 | 0.0 | 0.0 | 0.0 |
| Primary Th2 act | 0.0 | 0.3 | 0.7 | 0.0 |
| Primary Tr1 act | 0.0 | 0.7 | 0.0 | 0.0 |
| Primary Th1 rest | 0.0 | 0.1 | 0.0 | 0.0 |
| Primary Th2 rest | 0.0 | 0.4 | 0.0 | 0.0 |
| Primary Tr1 rest | 0.0 | 0.0 | 0.0 | 0.0 |
| CD45RA CD4 lymphocyte act | 0.0 | 5.4 | 0.8 | 0.0 |
| CD45RO CD4 lymphocyte act | 0.0 | 1.5 | 1.6 | 0.0 |
| CD8 lymphocyte act | 0.0 | 0.7 | 0.0 | 0.0 |
| Secondary CD8 lymphocyte rest | 0.0 | 8.8 | 0.0 | 0.0 |
| Secondary CD8 lymphocyte act | 0.0 | 0.4 | 0.0 | 0.0 |
| CD4 lymphocyte none | 0.0 | 0.5 | 0.0 | 0.0 |
| 2ry Th1/Th2/Tr1_anti-CD95 CH11 | 0.0 | 0.0 | 0.0 | 0.0 |
| LAK cells rest | 2.7 | 11.8 | 6.1 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN gamma | 0.0 | 0.0 | 0.0 | 0.0 |
| LAK cells IL-2 + IL-18 | 0.0 | 0.0 | 0.0 | 0.0 |
| LAK cells PMA/ionomycin | 15.7 | 15.1 | 6.1 | 0.0 |
| NK Cells IL-2 rest | 0.0 | 3.4 | 0.0 | 0.0 |
| Two Way MLR 3 day | 0.0 | 2.2 | 0.9 | 0.0 |
| Two Way MLR 5 day | 0.0 | 0.8 | 0.0 | 0.0 |
| Two Way MLR 7 day | 13.2 | 1.1 | 2.9 | 0.0 |
| PBMC rest | 0.0 | 0.0 | 0.0 | 0.0 |
| PBMC PWM | 0.0 | 1.3 | 0.0 | 0.0 |
| PBMC PHA-L | 0.0 | 0.6 | 0.0 | 0.0 |
| Ramos (B cell) none | 0.0 | 0.0 | 0.0 | 0.0 |
| Ramos (B cell) ionomycin | 0.0 | 0.7 | 0.0 | 0.0 |
| B lymphocytes PWM | 0.0 | 0.0 | 0.0 | 0.0 |
| B lymphocytes CD40L and IL-4 | 0.0 | 0.9 | 0.0 | 0.0 |
| EOL-1 dbcAMP | 9.1 | 29.1 | 0.0 | 0.0 |
| EOL-1 dbcAMP PMA/ionomycin | 0.0 | 0.0 | 1.0 | 0.0 |
| Dendritic cells none | 13.8 | 4.1 | 0.7 | 0.0 |
| Dendritic cells LPS | 0.0 | 1.0 | 0.0 | 0.0 |
| Dendritic cells anti-CD40 | 3.3 | 0.5 | 1.6 | 0.0 |
| Monocytes rest | 0.0 | 0.4 | 0.0 | 0.0 |
| Monocytes LPS | 0.0 | 5.7 | 0.0 | 0.4 |
| Macrophages rest | 0.0 | 0.6 | 0.0 | 0.0 |
| Macrophages LPS | 0.0 | 5.4 | 0.8 | 0.0 |
| HUVEC none | 0.0 | 0.0 | 0.0 | 0.0 |
| HUVEC starved | 0.0 | 0.0 | 0.0 | 0.0 |
| HUVEC IL-1beta | 0.0 | 0.0 | 0.0 | 0.0 |
| HUVEC IFN gamma | 0.0 | 0.0 | 0.0 | 0.0 |
| HUVEC TNF alpha + IFN gamma | 0.0 | 0.0 | 0.6 | 0.0 |
| HUVEC TNF alpha + IL4 | 0.0 | 0.0 | 0.0 | 0.0 |
| HUVEC IL-11 | 0.0 | 0.4 | 0.0 | 0.0 |
| Lung Microvascular EC none | 0.0 | 0.4 | 0.0 | 0.0 |
| Lung Microvascular EC | 0.0 | 0.0 | 0.0 | 0.0 |
| TNFalpha + IL-1beta | | | | |
| Microvascular Dermal EC none | 0.0 | 0.0 | 0.0 | 0.0 |
| Microsvasular Dermal EC | 0.0 | 0.0 | 0.0 | 0.0 |
| TNFalpha + IL-1beta | | | | |
| Bronchial epithelium | 0.0 | 0.0 | 0.0 | 0.0 |
| TNFalpha + IL1beta | | | | |
| Small airway epithelium none | 0.0 | 0.0 | 0.0 | 0.0 |
| Small airway epithelium | 0.0 | 0.0 | 0.0 | 0.0 |
| TNFalpha + IL-1beta | | | | |
| Coronery artery SMC rest | 0.0 | 0.0 | 0.5 | 0.3 |
| Coronery artery SMC | 6.2 | 0.3 | 0.0 | 0.0 |
| TNFalpha + IL-1beta | | | | |
| Astrocytes rest | 100.0 | 100.0 | 100.0 | 54.3 |
| Astrocytes TNFalpha + IL-1beta | 74.2 | 97.3 | 97.9 | 100.0 |
| KU-812 (Basophil) rest | 0.0 | 0.0 | 0.0 | 0.0 |
| KU-812 (Basophil) PMA/ionomycin | 0.0 | 0.0 | 0.0 | 0.0 |
| CCD1106 (Keratinocytes) none | 0.0 | 0.0 | 0.0 | 0.0 |
| CCD1106 (Keratinocytes) | 0.0 | 0.0 | 0.0 | 0.0 |
| TNFalpha + IL-1beta | | | | |
| Liver cirrhosis | 4.6 | 2.6 | 5.1 | 0.6 |
| NCI-H292 none | 0.0 | 1.7 | 0.0 | 0.0 |
| NCI-H292 IL-4 | 0.0 | 0.0 | 0.0 | 0.0 |
| NCI-H292 IL-9 | 0.0 | 0.7 | 0.0 | 0.0 |
| NCI-H292 IL-13 | 0.0 | 0.9 | 0.0 | 0.0 |
| NCI-H292 IFN gamma | 0.0 | 0.5 | 0.0 | 0.0 |
| HPAEC none | 0.0 | 0.0 | 0.0 | 0.0 |
| HPAEC TNF alpha + IL-1beta | 0.0 | 0.0 | 0.0 | 0.0 |
| Lung fibroblast none | 31.4 | 95.9 | 62.9 | 26.2 |
| Lung fibroblast | 22.2 | 48.6 | 25.2 | 28.3 |
| TNF alpha + IL-1 beta | | | | |
| Lung fibroblast IL-4 | 19.1 | 27.4 | 23.3 | 16.0 |
| Lung fibroblast IL-9 | 23.5 | 24.0 | 20.4 | 9.3 |
| Lung fibroblast IL-13 | 4.5 | 11.9 | 15.0 | 4.3 |
| Lung fibroblast IFN gamma | 15.7 | 55.9 | 29.9 | 25.2 |
| Dermal fibroblast CCD1070 rest | 0.0 | 6.0 | 5.6 | 0.0 |
| Dermal fibroblast CCD1070 TNF alpha | 0.0 | 2.7 | 0.8 | 1.1 |
| Dermal fibroblast CCD1070 IL-1 beta | 0.0 | 5.6 | 0.7 | 1.6 |
| Dermal fibroblast IFN gamma | 8.5 | 30.6 | 20.0 | 4.9 |
| Dermal fibroblast IL-4 | 4.1 | 30.8 | 22.7 | 13.5 |
| Dermal Fibroblasts rest | 8.0 | 54.3 | 20.7 | 15.8 |
| Neutrophils TNFa + LPS | 0.0 | 0.9 | 1.2 | 0.0 |
| Neutrophils rest | 0.0 | 0.0 | 0.0 | 0.0 |
| Colon | 4.0 | 4.6 | 7.9 | 4.8 |
| Lung | 0.0 | 2.8 | 1.6 | 0.0 |
| Thymus | 0.0 | 0.0 | 2.0 | 0.0 |
| Kidney | 4.9 | 7.8 | 10.2 | 0.6 |

CNS_neurodegeneration_v1.0 Summary: Ag6428/Ag6435/Ag6447 Three experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6432, Ag6425 Expression of this gene is low/undetectable (CTs>35) across all of the samples on this panel (data not shown).

General_screening_panel_v1.6 Summary: Ag6425/Ag6428/Ag6435 Four experiments with seven different probe and primer sets are in very good agreement. Highest expression of this gene is detected in kidney, a ovarian cancer IGROV-1 cell line and brain cancer SNB-19 cell lines (CTs=25-30). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6432/Ag6447 Expression of this gene is low/undetectable (CTs>34.9) across all of the samples on this panel (data not shown).

Panel 4.1D Summary: Ag6425/Ag6428/Ag6435/Ag6447 Four experiments with different probe and primer sets are in excellent agreement. Highest expression of this gene is detected in both resting and cytokine activated astrocytes (CTs31-34.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Ag6432 Expression of this gene is low/undetectable (CTs>35) across all of the samples on this panel (data not shown).

AW. CG56054-16: Integrin Alpha 7-Like Protein.

Expression of gene CG56054-16 was assessed using the primer-probe sets Ag6427, Ag6434, Ag6435 and Ag6447, described in Tables AWA, AWB, AWC and AWD. Results of the RTQ-PCR runs are shown in Tables AWE, AWF and AWG.

TABLE AWA

Probe Name Ag6427

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1301 | 678 |
| Probe | TET-5′-ccttcacaggtgctggagggc-3′ | 21 | 1341 | 679 |
| Reverse | 5′-ccctggatgcccatc-3′ | 15 | 1391 | 680 |

TABLE AWB

Probe Name Ag6434

| | | | Start | |
|---|---|---|---|---|
| Primers | Sequences | Length | Postion | SEQ ID No |
| Forward | 5′-cctttgatggtgatgqgaa-3′ | 19 | 1279 | 681 |
| Probe | TET-5′-cttcatctaccatqggagcagcctg-3′-TAMRA | 25 | 1301 | 682 |
| Reverse | 5′-gctcgggatgcccac-3′ | 15 | 1368 | 683 |

TABLE AWC

Probe Name Ag6435

| | | | | |
|---|---|---|---|---|
| Primers | Sequences | Length | Start Postion | SEQ ID No |

| | | | | |
|---|---|---|---|---|
| Forward | 5′-ggccagggtggagct-3′ | 15 | 731 | 684 |
| Probe | TET-5′-acctggcacacctggacgacg-3′-TAMRA | 21 | 766 | 685 |
| Reverse | 5′-cagggaccgggatga-3′ | 15 | 829 | 686 |

TABLE AWD

Probe Name Ag6447

| | | | Start | |
|---|---|---|---|---|
| Primers | Sequences | Length | Position | SEQ ID No |
| Forward | 5′gacgacggtccctacga-3′ | 17 | 780 | 687 |
| Probe | TET-5′-tcatcccggtccctgccaa-3′-TAMRA | 19 | 829 | 688 |
| Reverse | 5′-gtcaatagagaagccaaagtagct-3′ | 24 | 849 | 689 |

TABLE AWE
CNS_neurodegeneration_v1.0

| | Rel. Exp. (%) | Rel. Exp. (%) | Rel. Exp. (%) |
|---|---|---|---|
| | Ag6434, | Ag6435, | Ag6447, |
| | Run | Run | Run |
| Tissue Name | 269253996 | 269253997 | 269254007 |
| AD 1 Hippo | 17.3 | 17.1 | 18.8 |
| AD 2 Hippo | 33.0 | 27.9 | 10.4 |
| AD 3 Hippo | 3.4 | 4.8 | 0.0 |
| AD 4 Hippo | 9.0 | 18.3 | 4.6 |
| AD 5 hippo | 66.4 | 46.7 | 11.0 |
| AD 6 Hippo | 100.0 | 100.0 | 100.0 |
| Control 2 | 23.3 | 8.5 | 3.1 |
| Hippo | | | |
| Control 4 | 26.6 | 29.9 | 43.8 |
| Hippo | | | |
| Control | 7.0 | 5.2 | 5.3 |
| (Path) 3 | | | |
| Hippo | | | |
| AD 1 | 13.7 | 12.8 | 9.0 |
| Temporal | | | |
| Ctx | | | |
| AD 2 | 35.8 | 45.1 | 21.0 |
| Temporal | | | |
| Ctx | | | |
| AD 3 | 7.2 | 4.1 | 3.9 |
| Temporal | | | |
| Ctx | | | |
| AD 4 | 6.7 | 6.8 | 7.7 |
| Temporal | | | |
| ctx | | | |
| AD 5 Inf | 21.9 | 1.6 | 23.7 |
| Temporal | | | |
| Ctx | | | |
| AD 5 | 31.6 | 33.2 | 11.4 |
| Sup Temporal | | | |
| Ctx | | | |
| AD 6 Inf | 52.9 | 52.1 | 88.9 |
| Temporal | | | |
| Ctx | | | |
| AD 6 Sup | 71.2 | 37.6 | 61.1 |
| Temporal | | | |
| Ctx | | | |
| Control 1 | 10.3 | 6.7 | 2.8 |
| Temporal | | | |
| Ctx | | | |
| Control 2 | 16.2 | 7.3 | 16.0 |
| Temporal | | | |
| Ctx | | | |
| Control 3 | 8.5 | 4.4 | 3.1 |
| Temporal | | | |
| Ctx | | | |
| Control 4 Temporal | 13.6 | 11.7 | 13.6 |
| Ctx | | | |
| Control | 29.9 | 24.8 | 13.8 |
| (Path) 1 | | | |
| Temporal | | | |
| Ctx | | | |
| Control | 13.2 | 9.8 | 2.6 |
| (Path) 2 | | | |
| Temporal | | | |
| Ctx | | | |
| Control | 9.2 | 3.5 | 6.3 |
| (Path) 3 | | | |
| Temporal | | | |
| Ctx | | | |
| Control | 13.8 | 14.8 | 7.0 |
| (Path) 4 | | | |
| Temporal | | | |
| Ctx | | | |
| AD 1 | 8.4 | 15.0 | 0.0 |
| Occipital Ctx | | | |
| AD 2 | 0.0 | 0.0 | 0.0 |
| Occipital Ctx | | | |
| (Missing) | | | |
| AD 3 | 3.8 | 8.0 | 0.0 |
| Occipital Ctx | | | |
| AD 4 | 1.4 | 6.8 | 3.5 |
| Occipital Ctx | | | |
| AD 5 | 21.3 | 12.7 | 3.8 |
| Occipital Ctx | | | |
| AD 6 | 15.5 | 5.9 | 8.5 |
| Occipital Ctx | | | |
| Control 1 | 5.5 | 4.1 | 1.3 |
| Occipital Ctx | | | |
| Control 2 | 33.7 | 20.3 | 13.7 |
| Occipital Ctx | | | |
| Control 3 | 3.0 | 7.5 | 5.0 |
| Occipital Ctx | | | |
| Control 4 | 8.1 | 3.3 | 1.3 |
| OccipitaI Ctx | | | |
| Control | 39.0 | 25.9 | 12.1 |
| (Path) 1 | | | |
| Occipital Ctx | | | |
| Control | 4.2 | 7.4 | 13.2 |
| (Path) 2 | | | |
| Occipital Ctx | | | |
| Control | 3.2 | 2.3 | 9.4 |
| (Path) 3 | | | |
| Occipital Ctx | | | |
| Control | 9.3 | 21.0 | 20.4 |
| (Path) 4 | | | |
| Occipital Ctx | | | |
| Control 1 | 10.1 | 12.5 | 5.0 |
| Parietal Ctx | | | |
| Control 2 | 43.5 | 41.2 | 25.5 |
| Parietal Ctx | | | |
| Control 3 | 15.9 | 13.2 | 16.7 |
| Parietal Ctx | | | |
| Control | 24.8 | 22.5 | 4.2 |
| (Path) 1 | | | |
| Parietal Ctx | | | |
| Control | 22.1 | 26.8 | 14.4 |
| (Path) 2 | | | |
| Parietal Ctx | | | |
| Control | 9.3 | 7.5 | 5.9 |
| (Path) 3 | | | |
| Parietal Ctx | | | |
| Control | 34.6 | 20.6 | 9.4 |
| (Path) 4 | | | |
| Parietal Ctx | | | |

TABLE AWF
General_screening_panel_v1.6

| | Rel.Exp. (%) | Rel.Exp. (%) |
|---|---|---|
| | Ag6434, Run | Ag6435, Run |
| Tissue Name | 277222451 | 277223167 |
| Adipose | 9.5 | 13.2 |
| Melanoma* Hs688(A).T | 0.9 | 0.9 |
| Melanoma* Hs688(B).T | 3.7 | 1.9 |
| Melanoma* M14 | 0.7 | 0.0 |
| Melanoma* LOXIMVI | 0.0 | 0.0 |
| Melanoma* SK-MEL-5 | 14.7 | 4.4 |
| Squamous cell carcinoma SCC-4 | 0.0 | 0.0 |
| Testis Pool | 5.7 | 10.0 |
| Prostate ca.* (bone met) PC-3 | 1.5 | 1.8 |
| Prostate Pool | 4.2 | 10.0 |
| Placenta | 0.5 | 0.3 |
| Uterus Pool | 2.5 | 16.2 |
| Ovarian ca. OVCAR-3 | 0.8 | 0.4 |
| Ovarian ca. SK-OV-3 | 0.8 | 0.9 |
| Ovarian ca. OVCAR-4 | 0.5 | 0.0 |
| Ovarian ca. OVCAR-5 | 2.9 | 0.3 |
| Ovarian ca. IGROV-1 | 73.7 | 27.0 |
| Ovarian ca. OVCAR-8 | 20.7 | 7.6 |
| Ovary | 4.0 | 4.5 |
| Breast ca. MCF-7 | 0.5 | 0.0 |
| Breast ca. MDA-MB-231 | 0.5 | 0.0 |
| Breast ca. BT 549 | 0.5 | 0.0 |
| Breast ca. T47D | 0.0 | 0.0 |
| Breast ca. MDA-N | 0.0 | 0.7 |
| Breast Pool | 9.6 | 42.9 |
| Trachea | 5.3 | 8.3 |
| Lung | 1.3 | 3.9 |
| Fetal Lung | 5.0 | 8.0 |
| Lung ca. NCI-N417 | 3.0 | 0.2 |
| Lung ca. LX-1 | 4.3 | 0.9 |
| Lung ca. NCI-H146 | 0.0 | 0.0 |
| Lung ca. SHP-77 | 4.9 | 0.2 |
| Lung ca. A549 | 0.7 | 0.0 |
| Lung ca. NCI-H526 | 0.0 | 0.0 |
| Lung ca. NCI-H23 | 3.1 | 0.6 |
| Lung ca. NCI-H460 | 0.0 | 0.0 |
| Lung ca. HOP-62 | 0.0 | 0.0 |
| Lung ca. NCI-H522 | 1.4 | 0.0 |
| Liver | 0.0 | 0.0 |
| Fetal Liver | 0.5 | 0.3 |
| Liver ca. HepG2 | 0.5 | 0.0 |
| Kidney Pool | 22.8 | 100.0 |
| Fetal Kidney | 2.4 | 12.1 |
| Renal ca. 786-0 | 0.0 | 0.0 |
| Renal ca. A498 | 0.0 | 0.0 |
| Renal ca. ACHN | 0.7 | 0.0 |
| Renal ca. UO-31 | 0.0 | 0.0 |
| Renal ca. TK-10 | 3.0 | 0.7 |
| Bladder | 3.4 | 6.6 |
| Gastric ca. (liver met.) | 1.1 | 0.0 |
| NCI-N87 | | |
| Gastric ca. KATO III | 0.0 | 0.3 |
| Colon ca. SW-948 | 0.0 | 0.0 |
| Colon ca. SW480 | 28.3 | 4.4 |
| Colon ca.* (SW480 met) SW620 | 11.7 | 1.7 |
| Colon ca. HT29 | 0.0 | 0.0 |
| Colon ca. HCT-116 | 5.0 | 0.5 |
| Colon ca. CaCo-2 | 14.9 | 7.6 |
| Colon cancer tissue | 9.2 | 5.6 |
| Colon ca. SW1116 | 2.2 | 1.1 |
| Colon ca. Colo-205 | 0.0 | 0.0 |
| Colon ca. SW-48 | 1.4 | 0.0 |
| Colon Pool | 14.2 | 44.8 |
| Small Intestine Pool | 7.4 | 26.8 |
| Stomach Pool | 9.2 | 24.0 |
| Bone Marrow Pool | 4.6 | 25.9 |
| Fetal Heart | 11.3 | 31.6 |
| Heart Pool | 15.2 | 23.5 |
| Lymph Node Pool | 14.1 | 64.6 |
| Fetal Skeletal Muscle | 33.0 | 46.7 |
| Skeletal Muscle Pool | 21.2 | 24.7 |
| Spleen Pool | 1.2 | 2.4 |
| Thymus Pool | 6.1 | 18.4 |
| CNS cancer (glio/astro) | 10.4 | 5.8 |
| U87-MG | | |
| CNS cancer (glio/astro) | 3.4 | 1.5 |
| U-118-MG | | |
| CNS cancer (neuro; met) | 1.8 | 0.7 |
| SK-N-AS | | |
| CNS cancer (astro) | 0.0 | 0.2 |
| SF-539 | | |
| CNS cancer (astro) | 12.0 | 3.1 |
| SNB-75 | | |
| CNS cancer (glio) | 100.0 | 12.8 |
| SNB-19 | | |
| CNS cancer (glio) | 7.7 | 0.0 |
| SF-295 | | |
| Brain (Amygdala) Pool | 5.5 | 7.9 |
| Brain (cerebellum) | 11.0 | 1.8 |
| Brain (fetal) | 6.9 | 8.4 |
| Brain (Hippocampus) Pool | 8.5 | 9.9 |
| Cerebral Cortex Pool | 6.8 | 1.8 |
| Brain (Substantia nigra) Pool | 5.2 | 4.2 |
| Brain (Thalamus) Pool | 6.8 | 9.1 |
| Brain (whole) | 6.8 | 3.3 |
| Spinal Cord Pool | 6.4 | 13.1 |
| Adrenal Gland | 8.4 | 7.4 |
| Pituitary gland Pool | 0.6 | 1.8 |
| Salivary Gland | 1.6 | 2.3 |
| Thyroid (female) | 2.6 | 3.3 |
| Pancreatic ca. CAPAN2 | 0.9 | 0.5 |
| Pancreas Pool | 0.8 | 3.5 |

TABLE AWG
Panel 4.1D

| | Rel. Exp. (%) | Rel. Exp. (%) | Rel. Exp. (%) |
|---|---|---|---|
| | Ag6434, | Ag6435, | Ag6447, |
| | Run | Run | Run |
| Tissue Name | 268713326 | 268713480 | 268761806 |
| Secondary Th1 | 0.0 | 0.0 | 0.0 |
| act | | | |
| Secondary Th2 | 0.0 | 0.0 | 0.0 |
| act | | | |
| Secondary Tr1 | 0.0 | 0.0 | 0.0 |
| act | | | |
| Secondary Th1 | 0.0 | 0.0 | 0.0 |
| rest | | | |
| Secondary Th2 | 0.0 | 0.7 | 0.0 |
| rest | | | |
| Secondary Tr1 | 0.0 | 0.0 | 0.0 |
| rest | | | |
| Primary Th1 act | 0.0 | 0.0 | 0.0 |
| Primary Th2 act | 0.0 | 0.7 | 0.0 |
| Primary Tr1 act | 0.0 | 0.0 | 0.0 |
| Primary Th1 rest | 0.0 | 0.0 | 0.0 |
| Primary Th2 rest | 0.0 | 0.0 | 0.0 |
| Primary Tr1 rest | 0.0 | 0.0 | 0.0 |
| CD45RA CD4 | 0.0 | 0.8 | 0.0 |
| lymphocyte act | | | |
| CD45RO CD4 | 3.9 | 1.6 | 0.0 |
| lymphocyte act | | | |
| CD8 | 0.0 | 0.0 | 0.0 |
| Lymphocyte act | | | |
| Secondary CD8 | 0.0 | 0.0 | 0.0 |
| lymphocyte rest | | | |
| Secondary CD8 | 0.0 | 0.0 | 0.0 |
| lymphocyte act | | | |
| CD4 | 0.0 | 0.0 | 0.0 |
| lymphocyte | | | |
| none | | | |
| 2ry | 0.0 | 0.0 | 0.0 |
| Th1/Th2/Tr1_{—} | | | |
| anti-CD95 CH11 | | | |
| LAK cells rest | 7.9 | 6.1 | 0.0 |
| LAK cells IL-2 | 0.0 | 0.0 | 0.0 |
| LAK cells IL- | 0.0 | 0.0 | 0.0 |
| 2 + IL-12 | | | |
| LAK cells IL- | 0.0 | 0.0 | 0.0 |
| 2 + IFN gamma | | | |
| LAK cells IL-2 + | 0.0 | 0.0 | 0.0 |
| IL-18 | | | |
| LAK cells | 7.0 | 6.1 | 0.0 |
| PMA/ionomycin | | | |
| NK Cells IL-2 | 0.0 | 0.0 | 0.0 |
| rest | | | |
| Two Way MLR | 0.0 | 0.9 | 0.0 |
| 3 day | | | |
| Two Way MLR | 0.0 | 0.0 | 0.0 |
| 5 day | | | |
| Two Way MLR | 0.0 | 2.9 | 0.0 |
| 7day | | | |
| PBMC rest | 0.0 | 0.0 | 0.0 |
| PBMC PWM | 0.0 | 0.0 | 0.0 |
| PBMC PHA-L | 0.0 | 0.0 | 0.0 |
| Ramos (B cell) | 0.0 | 0.0 | 0.0 |
| none | | | |
| Ramos (B cell) | 0.0 | 0.0 | 0.0 |
| ionomycin | | | |
| B lymphocytes | 0.0 | 0.0 | 0.0 |
| PWM | | | |
| B lymphocytes | 0.0 | 0.0 | 0.0 |
| CD40L and IL-4 | | | |
| EOL-1 dbcAMP | 4.4 | 0.0 | 0.0 |
| EOL-1 dbcAMP | 0.0 | 1.0 | 0.0 |
| PMA/ionomycin | | | |
| Dendritic cells | 4.5 | 0.7 | 0.0 |
| none | | | |
| Dendritic cells | 0.0 | 0.0 | 0.0 |
| LPS | | | |
| Dendritic cells | 0.0 | 1.6 | 0.0 |
| anti-CD40 | | | |
| Monocytes rest | 0.0 | 0.0 | 0.0 |
| Monocytes LPS | 5.9 | 0.0 | 0.4 |
| Macrophages | 0.0 | 0.0 | 0.0 |
| rest | | | |
| Macrophages | 9.1 | 0.8 | 0.0 |
| LPS | | | |
| HUVEC none | 0.0 | 0.0 | 0.0 |
| HUVEC starved | 0.0 | 0.0 | 0.0 |
| HUVEC IL-1beta | 0.0 | 0.0 | 0.0 |
| HUVEC IFN | 0.0 | 0.0 | 0.0 |
| gamma | | | |
| HUVEC TNF | 0.0 | 0.6 | 0.0 |
| alpha + IFN | | | |
| gamma | | | |
| HUVEC TNF | 0.0 | 0.0 | 0.0 |
| alpha + IL4 | | | |
| HUVEC IL-11 | 0.0 | 0.0 | 0.0 |
| Lung | 0.0 | 0.0 | 0.0 |
| Microvascular | | | |
| EC none | | | |
| Lung | 0.0 | 0.0 | 0.0 |
| Microvascular | | | |
| EC TNFalpha + | | | |
| IL-1beta | | | |
| Microvascular | 0.0 | 0.0 | 0.0 |
| Dermal EC none | | | |
| Microsvasular | 0.0 | 0.0 | 0.0 |
| Dermal EC | | | |
| TNFalpha + IL- | | | |
| 1beta | | | |
| Bronchial | 0.0 | 0.0 | 0.0 |
| epithelium | | | |
| TNFalpha + | | | |
| IL1beta | | | |
| Small airway | 0.0 | 0.0 | 0.0 |
| epithelium none | | | |
| Small airway | 0.0 | 0.0 | 0.0 |
| epithelium | | | |
| TNFalpha + IL- | | | |
| 1beta | | | |
| Coronery artery | 0.0 | 0.5 | 0.3 |
| SMC rest | | | |
| Coronery artery | 0.0 | 0.0 | 0.0 |
| SMC TNFalpha + | | | |
| IL-1beta | | | |
| Astrocytes rest | 100.0 | 100.0 | 54.3 |
| Astrocytes | 97.3 | 97.9 | 100.0 |
| TNFalpha + IL- | | | |
| 1beta | | | |
| KU-812 | 0.0 | 0.0 | 0.0 |
| (Basophil) rest | | | |
| KU-812 | 0.0 | 0.0 | 0.0 |
| (Basophil) | | | |
| PMA/ionomycin | | | |
| CCD1106 | 0.0 | 0.0 | 0.0 |
| (Keratinocytes) | | | |
| none | | | |
| CCD1106 | 0.0 | 0.0 | 0.0 |
| (Keratinocytes) | | | |
| TNFalpha + IL- | | | |
| 1beta | | | |
| Liver cirrhosis | 3.4 | 5.1 | 0.6 |
| NCI-H292 none | 0.0 | 0.0 | 0.0 |
| NCI-H292 IL-4 | 0.0 | 0.0 | 0.0 |
| NCI-H292 IL-9 | 0.0 | 0.0 | 0.0 |
| NCI-H292 IL-13 | 0.0 | 0.0 | 0.0 |
| NCI-H292 IFN | 0.0 | 0.0 | 0.0 |
| gamma | | | |
| HPAEC none | 0.0 | 0.0 | 0.0 |
| HPAEC TNF | 0.0 | 0.0 | 0.0 |
| alpha + IL-1beta | | | |
| Lung fibroblast | 72.7 | 62.9 | 26.2 |
| none | | | |
| Lung fibroblast | 36.6 | 25.2 | 28.3 |
| TNF alpha + IL-1 | | | |
| beta | | | |
| Lung fibroblast | 62.4 | 23.3 | 16.0 |
| IL-4 | | | |
| Lung fibroblast | 52.5 | 20.4 | 9.3 |
| IL-9 | | | |
| Lung fibroblast | 14.6 | 15.0 | 4.3 |
| 1L-13 | | | |
| Lung fibroblast | 41.5 | 29.9 | 25.2 |
| IFN gamma | | | |
| Dermal fibroblast | 5.1 | 5.6 | 0.0 |
| CCD1070 rest | | | |
| Dermal fibroblast | 7.2 | 0.8 | 1.1 |
| CCD1070 TNF | | | |
| alpha | | | |
| Dermal fibroblast | 0.0 | 0.7 | 1.6 |
| CCD1070 IL-1 | | | |
| beta | | | |
| Dermal fibroblast | 24.5 | 20.0 | 4.9 |
| IFN gamma | | | |
| Dermal fibroblast | 28.7 | 22.7 | 13.5 |
| IL-4 | | | |
| Dermal | 44.4 | 20.7 | 15.8 |
| Fibroblasts rest | | | |
| Neutrophils | 0.0 | 1.2 | 0.0 |
| TNFa + LPS | | | |
| Neutrophils rest | 0.0 | 0.0 | 0.0 |
| Colon | 4.1 | 7.9 | 4.8 |
| Lung | 0.0 | 1.6 | 0.0 |
| Thymus | 0.0 | 2.0 | 0.0 |
| Kidney | 8.1 | 10.2 | 0.6 |

CNS_neurodegeneration_v1.0 Summary: Ag6434/Ag6435/Ag6447 Three experiments with different probe and primer sets are in good agreements. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6427 Expression of this gene is low/undetectable (CTs>35) across all of the samples on this panel (data not shown).

General_screening_panel_v1.6 Summary: Ag6434 Highest expression of this gene is detected in a brain cancer SNB-19 cell lines (CT=31.9). In addition, moderate to low levels of expression of this gene is also seen in some of the colon, ovarian and brain cancer cell lines. Thus, expression of this gene may be used as a marker to detect the presence of colon, ovarian and brain cancers. Furthermore, therapeutic modulation of this gene may be useful in the treatment of these cancers.

Ag6435 Highest expression of this gene is detected in kidney (CT=30.6). Moderate levels of expression of this gene is seen in normal tissues represented by breast, testis, prostate, uterus, gastrointestinal tract, and tissues with metabolic/endocrine functions including adipose, heart, skeletal muscle, and adernal gland. Therefore, therapeutic modulation of this gene or its protein product may be useful in the treatment of diseases associated with these tissues, including obesity, diabetes and inflammatory bowel disease. In addition, moderate to low levels of expression of this gene is also seen in some regions of central nervous system, and some brain, colon and ovarian cancer cell lines.

Ag6427/Ag6447 Expression of this gene is low/undetectable (CTs>34.9) across all of the samples on this panel (data not shown).

Panel 4.1D Summary: Ag6434/Ag6435/Ag6447 Three experiments with different probe and primer sets are in excellent agreement. Highest expression of this gene is detected in both resting and cytokine activated astrocytes (CTs=31-34.8). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Ag6427 Expression of this gene is low/undetectable (CTs>35) across all of the samples on this panel (data not shown).

AX. CG56054-17: Integrin Alpha 7-Like Protein.

Expression of gene CG56054-17 was assessed using the primer-probe sets Ag6425, Ag6426, Ag6435, Ag6439, Ag6440 and Ag6447, described in Tables AXA, AXB, AXC, AXD, AXE and AXF. Results of the RTQ-PCR runs are shown in Tables AXG, AXH and AXI.

TABLE AXA

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 1499 | 690 |
| Probe | TET-5′-catcccgagctgggcccc-3′-TAMRA | 18 | 1531 | 691 |
| Reverse | 5′-gccctggatgcccat-3′ | 15 | 1550 | 692 |

TABLE AXB

Probe Name Ag6426

| | | | | |
|---|---|---|---|---|
| Primers | Sequences | Length | Start Position | SEQ ID No |

| | | | | |
|---|---|---|---|---|
| Forward | 5′-gtcactgggctgggatct-3′ | 18 | 1156 | 693 |
| Probe | TET-5′-ctctccggctctgcggctc-3′-TAMRA | 19 | 1254 | 695 |
| Reverse | 5′-actccttctgccaccaca-3′ | 18 | 1254 | 695 |

TABLE AXC

Probe Name Ag6435

| | | | | |
|---|---|---|---|---|
| Primers | Sequences | Length | Start Position | SEQ ID No |

| | | | | |
|---|---|---|---|---|
| Froward | 5′-ggccagggtggagct-3′ | 15 | 731 | 696 |
| Probe | TET-5′-acctggcacacctggacgacg-3′-TAMRA | 21 | 766 | 697 |
| Reverse | 5′-cagggaccgggatga-3′ | 115 | 829 | 698 |

TABLE AXD

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 1253 | 699 |
| Probe | TET-5′-ccctggtgggtcatcctcctg-3′-TAMRA | 21 | 1273 | 700 |
| Reverse | 5′-gaagaatcccatcttccacag-3′ | 21 | 1339 | 701 |

TABLE AXE

Probe Name Ag6440

| | | | | |
|---|---|---|---|---|
| Primers | Sequences | Length | Start Position | SEQ ID No |

| | | | | |
|---|---|---|---|---|
| Forward | 5′-accatcctgaggaacaactg-3′ | 20 | 1456 | 702 |
| Probe | TET-5′ctgacgggcatcccgagct-3′-TAMRA | 19 | 1523 | 703 |
| Reverse | 5′-ccctggatgcccatc-3′ | 15 | 1549 | 704 |

TABLE AXF

Probe Name Ag6447

| | | | Start | |
|---|---|---|---|---|
| Primers | Sequences | Length | Position | SEQ ID No |
| Forward | 5′-gacgacggtccctacga-3′ | 17 | 780 | 705 |
| Probe | TET-5′-tcatcccqgtccctgccaa-3′-TAMRA | 19 | 829 | 706 |
| Reverse | 5′-gtcaatagagaagccaaagtagct-3′ | 24 | 849 | 707 |

TABLE AXG
CNS_neurodegeneration_v1.0

| | Rel. Exp. | Rel. Exp. | Rel. Exp. | Rel. Exp. | Rel. Exp. |
|---|---|---|---|---|---|
| | (%) Ag6425, | (%) Ag6435, | (%) Ag6439, | (%) Ag6440, | (%) Ag6447, |
| | Run | Run | Run | Run | Run |
| Tissue Name | 266937076 | 269253997 | 269254002 | 269254003 | 269254007 |
| AD 1 Hippo | 24.1 | 17.1 | 21.6 | 18.9 | 18.8 |
| AD 2 Hippo | 48.0 | 27.9 | 28.9 | 61.1 | 10.4 |
| AD 3 Hippo | 6.5 | 4.8 | 6.1 | 9.7 | 0.0 |
| AD 4 Hippo | 13.8 | 18.3 | 17.6 | 23.3 | 4.6 |
| AD 5 Hippo | 52.9 | 46.7 | 42.6 | 34.6 | 11.0 |
| AD 6 Hippo | 100.0 | 100.0 | 100.0 | 100.0 | 100.0 |
| Control 2 Hippo | 10.6 | 8.5 | 32.5 | 29.9 | 3.1 |
| Control 4 Hippo | 51.8 | 29.9 | 37.9 | 54.7 | 43.8 |
| Control (Path) 3 Hippo | 9.8 | 5.2 | 6.4 | 5.8 | 5.3 |
| AD 1 Temporal Ctx | 10.1 | 12.8 | 24.5 | 12.6 | 9.0 |
| AD 2 Temporal Ctx | 33.7 | 45.1 | 27.5 | 59.0 | 21.0 |
| AD 3 Temporal Ctx | 0.0 | 4.1 | 9.0 | 17.1 | 3.9 |
| AD 4 Temporal Ctx | 12.8 | 6.8 | 30.4 | 29.9 | 7.7 |
| AD 5 Inf Temporal Ctx | 59.0 | 1.6 | 41.8 | 41.8 | 23.7 |
| AD 5 Sup Temporal Ctx | 21.9 | 33.2 | 38.7 | 39.2 | 11.4 |
| AD 6 Inf Temporal Ctx | 73.7 | 52.1 | 47.6 | 48.6 | 88.9 |
| AD 6 Sup Temporal Ctx | 50.3 | 37.6 | 50.3 | 17.0 | 61.1 |
| Control 1 Temporal Ctx | 11.9 | 6.7 | 24.0 | 23.3 | 2.8 |
| Control 2 Temporal Ctx | 18.6 | 7.3 | 14.9 | 43.5 | 16.0 |
| Control 3 Temporal Ctx | 6.0 | 4.4 | 16.5 | 9.2 | 3.1 |
| Control 3 Temporal Ctx | 25.7 | 11.7 | 23.8 | 30.1 | 13.6 |
| Control (Path) 1 | 18.0 | 24.8 | 39.8 | 51.1 | 13.8 |
| Temporal Ctx | | | | | |
| Control (Path) 2 | 18.4 | 9.8 | 24.8 | 7.2 | 2.6 |
| Temporal Ctx | | | | | |
| Control (Path) 3 | 5.6 | 3.5 | 11.9 | 9.9 | 6.3 |
| Temporal Ctx | | | | | |
| Control (Path) 4 | 16.8 | 14.8 | 21.6 | 14.9 | 7.0 |
| Temporal Ctx | | | | | |
| AD 1 Occipital Ctx | 11.9 | 15.0 | 16.0 | 5.8 | 0.0 |
| AD 2 Occipital Ctx | 0.0 | 0.0 | 0.0 | 0.0 | 0.0 |
| (Missing) | | | | | |
| AD 3 Occipital Ctx | 8.3 | 8.0 | 10.2 | 7.8 | 0.0 |
| AD 4 Occipital Ctx | 5.8 | 6.8 | 18.6 | 35.4 | 3.5 |
| AD 5 Occipital Ctx | 25.2 | 12.7 | 22.7 | 16.6 | 3.8 |
| AD 6 Occipital Ctx | 19.8 | 5.9 | 22.1 | 23.5 | 8.5 |
| Control 1 Occipital Ctx | 6.6 | 4.1 | 7.2 | 15.2 | 1.3 |
| Control 2 Occipital Ctx | 15.7 | 20.3 | 29.3 | 35.8 | 13.7 |
| Control 3 Occipital Ctx | 5.7 | 7.5 | 19.2 | 4.4 | 5.0 |
| Control 4 Occipital Ctx | 21.6 | 3.3 | 13.6 | 12.9 | 1.3 |
| Control (Path) 1 | 28.3 | 25.9 | 39.5 | 22.4 | 12.1 |
| Occipital Ctx | | | | | |
| Control (Path) 2 | 49.7 | 7.4 | 7.0 | 5.0 | 13.2 |
| Occipital Ctx | | | | | |
| Control (Path) 3 | 0.0 | 2.3 | 5.9 | 6.7 | 9.4 |
| Occipital Ctx | | | | | |
| Control (Path) 4 | 6.6 | 21.0 | 11.4 | 11.9 | 20.4 |
| Occipital Ctx | | | | | |
| Control 1 Parietal Ctx | 8.8 | 12.5 | 15.7 | 33.2 | 5.0 |
| Control 2 Parietal Ctx | 14.5 | 41.2 | 37.1 | 17.4 | 25.5 |
| Control 3 Parietal Ctx | 19.9 | 13.2 | 10.8 | 21.6 | 16.7 |
| Control (Path) 1 | 37.6 | 22.5 | 37.9 | 47.3 | 4.2 |
| Parietal Ctx | | | | | |
| Control (Path) 2 | 16.6 | 26.8 | 18.7 | 17.1 | 14.4 |
| Parietal Ctx | | | | | |
| Control (Path) 3 | 0.0 | 7.5 | 12.0 | 11.7 | 5.9 |
| Parietal Ctx | | | | | |
| Control (Path) 4 | 18.2 | 20.6 | 27.9 | 29.3 | 9.4 |
| Parietal Ctx | | | | | |

TABLE AXH
General_screening_panel_v1.6

| | Rel. Exp. | Rel. Exp. (%) | Rel. Exp. | Rel. Exp. |
|---|---|---|---|---|
| | (%) Ag6425, | (%) Ag6435, | (%) Ag6439, | (%) Ag6440, |
| | Run | Run | Run | Run |
| Tissue Name | 277221721 | 277223167 | 277223175 | 277223177 |
| Adipose | 2.6 | 13.2 | 17.3 | 3.7 |
| Melanoma* Hs688(A).T | 0.0 | 0.9 | 0.4 | 0.0 |
| Melanoma* Hs688(B).T | 0.2 | 1.9 | 2.9 | 0.8 |
| Melanoma* M14 | 0.0 | 0.0 | 0.4 | 0.0 |
| Melanoma* LOXIMVI | 0.0 | 0.0 | 0.0 | 0.0 |
| Melanoma* SK-MEL-5 | 2.2 | 4.4 | 18.3 | 3.0 |
| Squamous cell carcinoma SCC-4 | 0.0 | 0.0 | 0.0 | 0.0 |
| Testis Pool | 3.5 | 10.0 | 9.1 | 3.0 |
| Prostate ca.* (bone met) PC-3 | 0.5 | 1.8 | 1.3 | 1.2 |
| Prostate Pool | 1.0 | 10.0 | 28.5 | 2.1 |
| Placenta | 0.0 | 0.3 | 0.5 | 0.0 |
| Uterus Pool | 1.5 | 16.2 | 5.3 | 2.3 |
| Ovarian ca. OVCAR-3 | 0.3 | 0.4 | 1.6 | 0.4 |
| Ovarian ca. SK-OV-3 | 0.2 | 0.9 | 1.3 | 0.5 |
| Ovarian ca. OVCAR-4 | 0.0 | 0.0 | 0.9 | 0.0 |
| Ovarian ca. OVCAR-5 | 1.3 | 0.3 | 1.4 | 4.2 |
| Ovarian ca. IGROV-1 | 100.0 | 27.0 | 69.3 | 100.0 |
| Ovarian ca. OVCAR-8 | 21.9 | 7.6 | 17.3 | 18.2 |
| Ovary | 0.3 | 4.5 | 2.8 | 0.8 |
| Breast ca. MCF-7 | 0.0 | 0.0 | 0.5 | 0.3 |
| Breast ca. MDA-MB-231 | 0.0 | 0.0 | 0.2 | 0.0 |
| Breast ca. BT 549 | 0.0 | 0.0 | 0.6 | 0.0 |
| Breast ca. T47D | 0.0 | 0.0 | 0.4 | 0.3 |
| Breast ca. MDA-N | 0.0 | 0.7 | 0.6 | 03 |
| Breast Pool | 4.1 | 42.9 | 12.2 | 3.5 |
| Trachea | 0.7 | 8.3 | 4.7 | 1.4 |
| Lung | 0.7 | 3.9 | 3.9 | 5.3 |
| Fetal Lung | 0.3 | 8.0 | 5.3 | 2.9 |
| Lung ca. NCI-N417 | 0.9 | 0.2 | 4.0 | 2.0 |
| Lung ca. LX-1 | 2.7 | 0.9 | 4.9 | 6.3 |
| Lung ca. NCI-H146 | 0.0 | 0.0 | 0.1 | 0.0 |
| Lung ca. SHP-77 | 0.4 | 0.2 | 4.5 | 0.8 |
| Lung ca. A549 | 2.6 | 0.0 | 0.6 | 2.2 |
| Lung ca. NCI-H526 | 0.0 | 0.0 | 0.4 | 0.3 |
| Lung ca. NCI-H23 | 1.0 | 0.6 | 2.9 | 2.3 |
| Lung ca. NCI-H460 | 0.0 | 0.0 | 0.0 | 0.0 |
| Lung ca. HOP-62 | 0.0 | 0.0 | 0.5 | 0.0 |
| Lung ca. NCI-H522 | 0.6 | 0.0 | 3.3 | 2.5 |
| Liver | 0.0 | 0.0 | 0.1 | 0.4 |
| Fetal Liver | 0.3 | 0.3 | 0.8 | 0.8 |
| Liver ca. HepG2 | 0.3 | 0.0 | 0.1 | 0.9 |
| Kidney Pool | 0.0 | 100.0 | 43.2 | 14.6 |
| Fetal Kidney | 0.0 | 12.1 | 5.8 | 3.4 |
| Renal ca. 786-0 | 0.0 | 0.0 | 0.3 | 0.0 |
| Renal ca. A498 | 1.8 | 0.0 | 0.5 | 3.8 |
| Renal ca. ACHN | 0.5 | 0.0 | 1.2 | 0.5 |
| Renal ca. UO-31 | 0.0 | 0.0 | 0.6 | 0.0 |
| Renal ca. TK-10 | 0.4 | 0.7 | 2.1 | 0.5 |
| Bladder | 0.0 | 6.6 | 8.3 | 0.9 |
| Gastric ca. (liver met.) | 0.0 | 0.0 | 1.1 | 0.8 |
| NCI-N87 | | | | |
| Gastric ca. KATO III | 0.5 | 0.3 | 0.4 | 0.4 |
| Colon ca. SW-948 | 1.5 | 0.0 | 0.3 | 2.2 |
| Colon ca. SW480 | 5.2 | 4.4 | 23.0 | 6.3 |
| Colon ca.* (SW480 | 4.8 | 1.7 | 6.1 | 7.2 |
| met) SW620 | | | | |
| Colon ca. HT29 | 0.0 | 0.0 | 0.0 | 0.3 |
| Colon ca. HCT-H6 | 0.2 | 0.5 | 2.1 | 0.6 |
| Colon ca. CaCo-2 | 3.6 | 7.6 | 18.3 | 6.5 |
| Colon cancer tissue | 3.3 | 5.6 | 7.7 | 4.4 |
| Colon ca. SW1116 | 3.0 | 1.1 | 1.8 | 2.1 |
| Colon ca. Colo-205 | 0.4 | 0.0 | 0.2 | 1.3 |
| Colon ca. SW-48 | 3.6 | 0.0 | 1.4 | 3.0 |
| Colon Pool | 5.0 | 44.8 | 25.5 | 8.1 |
| Small Intestine Pool | 1.7 | 26.8 | 12.8 | 2.0 |
| Stomach Pool | 2.3 | 24.0 | 8.5 | 4.2 |
| Bone Marrow Pool | 1.6 | 25.9 | 18.7 | 3.5 |
| Fetal Heart | 2.3 | 31.6 | 33.7 | 8.6 |
| Heart Pool | 7.0 | 23.5 | 33.7 | 10.7 |
| Lymph Node Pool | 6.1 | 64.6 | 19.9 | 6.7 |
| Fetal Skeletal Muscle | 5.2 | 46.7 | 19.1 | 19.2 |
| Skeletal Muscle Pool | 9.2 | 24.7 | 22.1 | 22.7 |
| Spleen Pool | 0.0 | 2.4 | 2.1 | 0.6 |
| Thymus Pool | 2.0 | 18.4 | 7.7 | 3.1 |
| CNS cancer (glio/astro) U87-MG | 1.5 | 5.8 | 10.9 | 2.2 |
| CNS cancer (glio/astro) U-118-MG | 0.3 | 1.5 | 3.8 | 0.8 |
| CNS cancer (neuro; met) SK-N-AS | 0.0 | 0.7 | 1.4 | 0.5 |
| CNS cancer (astro) SF-539 | 0.0 | 0.2 | 0.1 | 0.2 |
| CNS cancer (astro) SNB-75 | 1.1 | 3.1 | 11.7 | 2.8 |
| CNS cancer (glio) SNB-19 | 79.0 | 12.8 | 100.0 | 97.9 |
| CNS cancer (glio) SF-295 | 0.0 | 0.0 | 8.2 | 1.5 |
| Brain (Amygdala) Pool | 0.8 | 7.9 | 8.0 | 4.4 |
| Brain (cerebellum) | 0.4 | 1.8 | 8.8 | 1.2 |
| Brain (fetal) | 0.7 | 8.4 | 6.8 | 2.1 |
| Brain (Hippocampus) Pool | 3.2 | 9.9 | 11.0 | 4.3 |
| Cerebral Cortex Pool | 0.6 | 1.8 | 11.6 | 2.0 |
| Brain (Substantia nigra) Pool | 2.2 | 4.2 | 10.0 | 2.0 |
| Brain (Thalamus) Pool | 2.7 | 9.1 | 9.7 | 2.8 |
| Brain (whole) | 0.4 | 3.3 | 5.6 | 1.9 |
| Spinal Cord Pool | 2.3 | 13.1 | 12.2 | 4.2 |
| Adrenal Gland | 0.3 | 7.4 | 4.8 | 0.9 |
| Pituitary gland Pool | 0.0 | 1.8 | 1.4 | 0.6 |
| Salivary Gland | 0.0 | 2.3 | 1.1 | 0.0 |
| Thyroid (female) | 0.3 | 3.3 | 1.9 | 1.3 |
| Pancreatic ca. CAPAN2 | 0.0 | 0.5 | 0.7 | 0.6 |
| Pancreas Pool | 0.0 | 3.5 | 3.2 | 1.0 |

TABLE AXI
Panel 4.1D

| | Rel. Exp. (%) | Rel. Exp. (%) | Rel. Exp. (%) | Rel. Exp. (%) |
|---|---|---|---|---|
| | Ag6425, Run | Ag6435, Run | Ag6439, Run | Ag6447, Run |
| Tissue Name | 268713999 | 268713480 | 268760823 | 268761806 |
| Secondary Th1 act | 0.0 | 0.0 | 0.0 | 0.0 |
| Secondary Th2 act | 0.0 | 0.0 | 0.0 | 0.0 |
| Secondary Tr1 act | 0.0 | 0.0 | 0.0 | 0.0 |
| Secondary Th1 rest | 0.0 | 0.0 | 0.0 | 0.0 |
| Secondary Th2 rest | 0.0 | 0.7 | 0.0 | 0.0 |
| Secondary Tr1 rest | 0.0 | 0.0 | 0.0 | 0.0 |
| Primary Th1 act | 0.0 | 0.0 | 0.0 | 0.0 |
| Primary Th2 act | 0.0 | 0.7 | 0.0 | 0.0 |
| Primary Tr1 act | 0.0 | 0.0 | 0.0 | 0.0 |
| Primary Th1 rest | 0.0 | 0.0 | 1.2 | 0.0 |
| Primary Th2 rest | 0.0 | 0.0 | 0.0 | 0.0 |
| Primary Tr1 rest | 0.0 | 0.0 | 0.0 | 0.0 |
| CD45RA CD4 | 0.0 | 0.8 | 2.6 | 0.0 |
| lymphocyte act | | | | |
| CD45RO CD4 | 0.0 | 1.6 | 2.3 | 0.0 |
| lymphocyte act | | | | |
| CD8 lymphocyte act | 0.0 | 0.0 | 0.0 | 0.0 |
| Secondary CD8 | 0.0 | 0.0 | 0.0 | 0.0 |
| lymphocyte rest | | | | |
| Secondary CD8 | 0.0 | 0.0 | 0.0 | 0.0 |
| lymphocyte act | | | | |
| CD4 lymphocyte none | 0.0 | 0.0 | 0.0 | 0.0 |
| 2ry Th1/Th2/Tr1_anti- | 0.0 | 0.0 | 1.2 | 0.0 |
| CD95 CH11 | | | | |
| LAK cells rest | 2.7 | 6.1 | 15.2 | 0.0 |
| LAK cells IL-2 | 0.0 | 0.0 | 0.0 | 0.0 |
| LAK cells IL-2 + IL-12 | 0.0 | 0.0 | 0.0 | 0.0 |
| LAK cells IL-2 + IFN | 0.0 | 0.0 | 0.0 | 0.0 |
| gamma | | | | |
| LAK cells IL-2 + IL-18 | 0.0 | 0.0 | 0.0 | 0.0 |
| LAK cells | 15.7 | 6.1 | 9.0 | 0.0 |
| PMA/ionomycin | | | | |
| NK Cells IL-2 rest | 0.0 | 0.0 | 1.4 | 0.0 |
| Two Way MLR 3 day | 0.0 | 0.9 | 1.4 | 0.0 |
| Two Way MLR 5 day | 0.0 | 0.0 | 0.0 | 0.0 |
| Two Way MLR 7 day | 13.2 | 2.9 | 3.7 | 0.0 |
| PBMC rest | 0.0 | 0.0 | 0.0 | 0.0 |
| PBMC PWM | 0.0 | 0.0 | 0.0 | 0.0 |
| PBMC PHA-L | 0.0 | 0.0 | 0.0 | 0.0 |
| Ramos (B cell) none | 0.0 | 0.0 | 0.0 | 0.0 |
| Ramos (B cell) ionomycin | 0.0 | 0.0 | 0.0 | 0.0 |
| B lymphocytes PWM | 0.0 | 0.0 | 0.0 | 0.0 |
| B lymphocytes CD40L | 0.0 | 0.0 | 0.0 | 0.0 |
| and IL-4 | | | | |
| EOL-1 dbcAMP | 9.1 | 0.0 | 68.8 | 0.0 |
| EOL-1 dbcAMP | 0.0 | 1.0 | 1.8 | 0.0 |
| PMA/ionomycin | | | | |
| Dendritic cells none | 13.8 | 0.7 | 0.0 | 0.0 |
| Dendritic cells LPS | 0.0 | 0.0 | 0.0 | 0.0 |
| Dendritic cells anti- | 3.3 | 1.6 | 0.0 | 0.0 |
| CD40 | | | | |
| Monocytes rest | 0.0 | 0.0 | 0.0 | 0.0 |
| Monocytes LPS | 0.0 | 0.0 | 2.6 | 0.4 |
| Macrophages rest | 0.0 | 0.0 | 0.0 | 0.0 |
| Macrophages LPS | 0.0 | 0.8 | 9.2 | 0.0 |
| HUVEC none | 0.0 | 0.0 | 0.0 | 0.0 |
| HUVEC starved | 0.0 | 0.0 | 0.0 | 0.0 |
| HUVEC IL-1beta | 0.0 | 0.0 | 0.0 | 0.0 |
| HUVEC IFN gamma | 0.0 | 0.0 | 0.0 | 0.0 |
| HUVEC TNF alpha + IFN | 0.0 | 0.6 | 0.0 | 0.0 |
| gamma | | | | |
| HUVEC TNF alpha + IL4 | 0.0 | 0.0 | 0.0 | 0.0 |
| HUVEC IL-11 | 0.0 | 0.0 | 0.0 | 0.0 |
| Lung Microvascular EC | 0.0 | 0.0 | 0.0 | 0.0 |
| none | | | | |
| Lung Microvascular EC | 0.0 | 0.0 | 0.0 | 0.0 |
| TNFalpha + IL-1beta | | | | |
| Microvascular Dermal ECnone | 0.0 | 0.0 | 0.0 | 0.0 |
| Microsvasular Dermal EC | 0.0 | 0.0 | 0.0 | 0.0 |
| TNFalpha + IL-1beta | | | | |
| Bronchial epithelium | 0.0 | 0.0 | 0.0 | 0.0 |
| TNFalpha + IL1beta | | | | |
| Small airway epithelium none | 0.0 | 0.0 | 0.0 | 0.0 |
| Small airway epithelium | 0.0 | 0.0 | 0.0 | 0.0 |
| TNFalpha + IL-1beta | | | | |
| Coronery artery SMC rest | 0.0 | 0.5 | 0.0 | 0.3 |
| Coronery artery SMC | 6.2 | 0.0 | 0.0 | 0.0 |
| TNFalpha + IL-1beta | | | | |
| Astrocytes rest | 100.0 | 100.0 | 100.0 | 54.3 |
| Astrocytes TNFalpha + | 74.2 | 97.9 | 95.9 | 100.0 |
| IL-1beta | | | | |
| KU-812 (Basophil) rest | 0.0 | 0.0 | 0.0 | 0.0 |
| KU-812 (Basophil) | 0.0 | 0.0 | 0.0 | 0.0 |
| PMA/ionomycin | | | | |
| CCD1106 (Keratinocytes) | 0.0 | 0.0 | 0.0 | 0.0 |
| none | | | | |
| CCD1106 (Keratinocytes) | 0.0 | 0.0 | 0.0 | 0.0 |
| TNFalpha + IL-1beta | | | | |
| Liver cirrhosis | 4.6 | 5.1 | 8.5 | 0.6 |
| NCI-H292 none | 0.0 | 0.0 | 0.0 | 0.0 |
| NCI-H292 IL-4 | 0.0 | 0.0 | 0.0 | 0.0 |
| NCI-H292 IL-9 | 0.0 | 0.0 | 0.0 | 0.0 |
| NCI-H292 IL-13 | 0.0 | 0.0 | 0.0 | 0.0 |
| NCI-H292 IFN gamma | 0.0 | 0.0 | 0.0 | 0.0 |
| HPAEC none | 0.0 | 0.0 | 0.0 | 0.0 |
| HPAEC TNF alpha + IL-1 | 0.0 | 0.0 | 0.0 | 0.0 |
| beta | | | | |
| Lung fibroblast none | 31.4 | 62.9 | 94.0 | 26.2 |
| Lung fibroblast TNF | 22.2 | 25.2 | 62.9 | 28.3 |
| alpha + IL-1 beta | | | | |
| Lung fibroblast IL-4 | 19.1 | 23.3 | 34.9 | 16.0 |
| Lung fibroblast IL-9 | 23.5 | 20.4 | 96.6 | 9.3 |
| Lung fibroblast IL-13 | 4.5 | 15.0 | 13.4 | 4.3 |
| Lung fibroblast IFN | 15.7 | 29.9 | 89.5 | 25.2 |
| gamma | | | | |
| Dermal fibroblast | 0.0 | 5.6 | 4.1 | 0.0 |
| CCD1070 rest | | | | |
| Dermal fibroblast | 0.0 | 0.8 | 2.3 | 1.1 |
| CCD1070 TNF alpha | | | | |
| Dermal fibroblast | 0.0 | 0.7 | 0.0 | 1.6 |
| CCD1070 IL-1 beta | | | | |
| Dermal fibroblast IFN | 8.5 | 20.0 | 26.6 | 4.9 |
| gamma | | | | |
| Dermal fibroblast IL-4 | 4.1 | 22.7 | 25.5 | 13.5 |
| Dermal Fibroblasts | 8.0 | 20.7 | 47.3 | 15.8 |
| rest | | | | |
| Neutrophils TNFa + | 0.0 | 1.2 | 0.0 | 0.0 |
| LPS | | | | |
| Neutrophils rest | 0.0 | 0.0 | 0.0 | 0.0 |
| Colon | 4.0 | 7.9 | 8.4 | 4.8 |
| Lung | 0.0 | 1.6 | 2.1 | 0.0 |
| Thymus | 0.0 | 2.0 | 2.4 | 0.0 |
| Kidney | 4.9 | 10.2 | 5.2 | 0.6 |

CNS_neurodegeneration_v1.0 Summary: Ag6425/Ag6435/Ag6439/Ag6440/Ag6447 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6426 Expression of this gene is low/undetectable (CTs>34.9) across all of the samples on this panel (data not shown).

General_screening_panel_v1.6 Summary: Ag6425/Ag6435/Ag6439/Ag6440 Four experiments with seven different probe and primer sets are in very good agreement. Highest expression of this gene is detected in kidney, a ovarian cancer IGROV-1 cell line and brain cancer SNB-19 cell lines (CTs=25-30). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6447 Expression of this gene is low/undetectable (CTs>35) across all of the samples on this panel (data not shown).

Panel 4.1D Summary: Ag6425/Ag6435/Ag6439/Ag6447 Four experiments with different probe and primer sets are in excellent agreement. Highest expression of this gene is detected in both resting and cytokine activated astrocytes (CTs=31-34.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Ag6426/Ag6440 Expression of this gene is low/undetectable (CTs>35) across all of the samples on this panel (data not shown).

AY. CG56054-18: Integrin Alpha 7-Like Protein.

Expression of gene CG56054-18 was assessed using the primer-probe sets Ag4983, Ag6442, Ag6425, Ag6428, Ag6431, Ag6435, Ag6439, Ag6447, Ag6413 and Ag6964, described in Tables AYA, AYB, AYC, AYD, AYE, AYF, AYG, AYH, AYI and AYJ. Results of the RTQ-PCR runs are shown in Tables AYK, AYL, AYM, AYN, AYO and AYP.

TABLE AYA

Probe Name Ag4983

| | | | Start | |
|---|---|---|---|---|
| Primers | Sequences | Length | Position | SEQ ID No |
| Forward | 5′-ccaggtcaccttctacctcatc-3′ | 22 | 2342 | 708 |
| Probe | TET-5′-cttagcacctccgggatcagcatt-3′-TAMRA | 24 | 2364 | 709 |
| Reverse | 5′-aacagcagctctacctccagtt-3′ | 22 | 2398 | 710 |

TABLE AYB

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3′ | 22 | 2781 | 711 |
| Probe | TET-5′-ccacctgagcagcaggagcct-3′-TAMRA | 21 | 2820 | 712 |
| Reverse | 5′-gcgcagtccagggtg-3′ | 15 | 2966 | 713 |

TABLE AYC

Probe Name Ag6425

| | | | | |
|---|---|---|---|---|
| Primers | Sequences | Length | Start Position | SEQ ID No |

| | | | | |
|---|---|---|---|---|
| Forward | 5′-cggatgcacaccccat-3′ | 16 | 3296 | 714 |
| Probe | TET-5′-catcccqagctgggcccc-3′-TAMRA | 18 | 3328 | 715 |
| Reverse | 5′-gccctggatgcccat-3′ | 15 | 3347 | 716 |

TABLE AYD

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgqgagca-3′ | 20 | 1301 | 717 |
| Probe | TET-5′-ccttcacaggtgctggagggc-3′-TAMRA | 21 | 1341 | 718 |
| Reverse | 5′-agggagtaqccgaagctct-3′ | 19 | 1378 | 719 |

TABLE AYE

Probe Name Ag6431

| | | | Start | |
|---|---|---|---|---|
| Primer | Sequence | Length | Position | SEQ ID No |
| Forward | 5′-aaacatcaccctggactgc-3′ | 19 | 2900 | 720 |
| Probe | TET-5′-tggtgttcagctgcccacectacag-3′-TAMRA | 25 | 2941 | 721 |
| Reverse | 5′-ccgcgcggtcaaa-3′ | 13 | 2967 | 722 |

TABLE AYF

Probe Name Ag6435

| | | | | |
|---|---|---|---|---|
| Primers | Sequences | Length | Start Position | SEQ ID No |

| | | | | |
|---|---|---|---|---|
| Forward | 5′-ggccagggtggagct-3′ | 15 | 731 | 723 |
| Probe | TET-5′-acctggcacacctggacgacg-3′-TAMRA | 21 | 766 | 724 |
| Reverse | 5′-cagggaccgggatga-3′ | 15 | 829 | 725 |

TABLE AYG

Probe Name Ag6439

| | | | | |
|---|---|---|---|---|
| Primers | Sequences | Length | Start Position | SEQ ID No |

| | | | | |
|---|---|---|---|---|
| Forward | 5′-ctgtggtggcagaaggagt-3′ | 19 | 3157 | 726 |
| Probe | TET-5′-ccctggtgggtcatcctcctg-3′-TAMRA | 21 | 3177 | 727 |
| Reverse | 5′-gaagaatcccatcttccacag-3′ | 21 | 3243 | 728 |

TABLE AYI

Probe Name Ag6447

| | | | Start | |
|---|---|---|---|---|
| Primers | Sequences | Length | Position | SEQ ID No |
| Forward | 5′-gacgacggtccctacga-3′ | 17 | 780 | 729 |
| Probe | TET-5′-tcatcccggtccctqccaa-3′-TAMRA | 19 | 829 | 730 |
| Reverse | gtcaatagagaagccaaagtagct-3′ | 24 | 849 | 731 |

TABLE AYI

Probe Name Ag6413

| | | | | |
|---|---|---|---|---|
| Primers | Sequences | Length | Start Position | SEQ ID No |

| | | | | |
|---|---|---|---|---|
| Forward | 5′-ggtgaagacaagatctgccag-3′- | 21 | 1980 | 732 |
| Probe | TET-5′-tgtgaagacaagatctgccag-3′-TAMRA | 21 | 2031 | 733 |
| Reverse | 5′-gctgttgttccatccacatc-3′ | 20 | 2073 | 734 |

TABLE AYJ

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatqca-3′ | 15 | 2986 | 735 |
| Probe | TET-5′-actctacagctttgaccgcgcgg-3′-TAMRA | 23 | 2957 | 736 |
| Reverse | 5′-gccaactgtgtggtgttca-3′ | 19 | 2931 | 737 |

TABLE AYK
CNS_neurodegeneration_v1.0

| | Rel. | Rel. | Rel. | Rel. | Rel. | Rel. | Rel. | Rel. | Rel. |
|---|---|---|---|---|---|---|---|---|---|
| | Exp. (%) | Exp. (%) | Exp. (%) | Exp. (%) | Exp. (%) | Exp. (%) | Exp. (%) | Exp. (%) | Exp. (%) |
| | Ag4983, | Ag6413, | Ag6425, | Ag6428, | Ag6431, | Ag6435, | Ag6439, | Ag6442, | Ag6447, |
| Tissue | Run | Run | Run | Run | Run | Run | Run | Run | Run |
| Name | 218649223 | 269253983 | 266937076 | 266937081 | 268030722 | 269253997 | 269254002 | 264979298 | 269254007 |
| AD 1 | 23.7 | 24.8 | 24.1 | 18.0 | 18.8 | 17.1 | 21.6 | 19.2 | 18.8 |
| Hippo | | | | | | | | | |
| AD 2 | 41.2 | 52.9 | 48.0 | 32.3 | 28.7 | 27.9 | 28.9 | 49.7 | 10.4 |
| Hippo | | | | | | | | | |
| AD 3 | 8.9 | 6.4 | 6.5 | 3.7 | 7.5 | 4.8 | 6.1 | 20.4 | 0.0 |
| Hippo | | | | | | | | | |
| AD 4 | 14.8 | 25.5 | 13.8 | 10.7 | 18.8 | 18.3 | 17.6 | 5.6 | 4.6 |
| Hippo | | | | | | | | | |
| AD 5 | 44.8 | 41.8 | 52.9 | 53.2 | 38.4 | 46.7 | 42.6 | 57.4 | 11.0 |
| Hippo | | | | | | | | | |
| AD 6 | 100.0 | 100.0 | 100.0 | 100.0 | 100.0 | 100.0 | 100.0 | 90.1 | 100.0 |
| Hippo | | | | | | | | | |
| Control 2 | 24.3 | 36.1 | 10.6 | 18.7 | 29.5 | 8.5 | 32.5 | 28.5 | 3.1 |
| Hippo | | | | | | | | | |
| Control 4 | 42.9 | 43.8 | 51.8 | 27.0 | 32.3 | 29.9 | 37.9 | 86.5 | 43.8 |
| Hippo | | | | | | | | | |
| Control | 14.2 | 11.4 | 9.8 | 4.6 | 6.0 | 5.2 | 6.4 | 0.0 | 5.3 |
| (Path) 3 | | | | | | | | | |
| Hippo | | | | | | | | | |
| AD 1 | 23.3 | 15.9 | 10.1 | 12.9 | 17.1 | 12.8 | 24.5 | 16.8 | 9.0 |
| Temporal | | | | | | | | | |
| Ctx | | | | | | | | | |
| AD 2 | 41.5 | 47.3 | 33.7 | 31.0 | 39.8 | 45.1 | 27.5 | 21.6 | 21.0 |
| Temporal | | | | | | | | | |
| Ctx | | | | | | | | | |
| AD 3 | 9.5 | 9.8 | 0.0 | 6.0 | 11.3 | 4.1 | 9.0 | 5.7 | 3.9 |
| Temporal | | | | | | | | | |
| Ctx | | | | | | | | | |
| AD 4 | 30.6 | 39.0 | 12.8 | 20.2 | 25.3 | 6.8 | 30.4 | 8.7 | 7.7 |
| Temporal | | | | | | | | | |
| Ctx | | | | | | | | | |
| AD 5 Inf | 45.4 | 37.1 | 59.0 | 39.2 | 36.3 | 1.6 | 41.8 | 73.7 | 23.7 |
| Temporal | | | | | | | | | |
| Ctx | | | | | | | | | |
| AD 5 Sup | 51.1 | 39.0 | 21.9 | 42.0 | 32.3 | 33.2 | 38.7 | 55.9 | 11.4 |
| Temporal | | | | | | | | | |
| Ctx | | | | | | | | | |
| AD 6 Inf | 38.2 | 59.9 | 73.7 | 49.3 | 46.7 | 52.1 | 47.6 | 76.8 | 88.9 |
| Temporal | | | | | | | | | |
| Ctx | | | | | | | | | |
| AD 6 Sup | 43.8 | 48.6 | 50.3 | 48.3 | 50.3 | 37.6 | 50.3 | 59.9 | 61.1 |
| Temporal | | | | | | | | | |
| Ctx | | | | | | | | | |
| Control 1 | 12.2 | 23.0 | 11.9 | 12.9 | 15.6 | 6.7 | 24.0 | 46.7 | 2.8 |
| Temporal | | | | | | | | | |
| Ctx | | | | | | | | | |
| Control 2 | 14.2 | 32.5 | 18.6 | 18.2 | 17.4 | 7.3 | 14.9 | 50.0 | 16.0 |
| Temporal | | | | | | | | | |
| Ctx | | | | | | | | | |
| Control 3 | 15.1 | 15.3 | 6.0 | 9.6 | 14.5 | 4.4 | 16.5 | 9.5 | 3.1 |
| Temporal | | | | | | | | | |
| Ctx | | | | | | | | | |
| Control 3 | 23.7 | 25.0 | 25.7 | 15.2 | 13.1 | 11.7 | 23.8 | 13.6 | 13.6 |
| Temporal | | | | | | | | | |
| Ctx | | | | | | | | | |
| Control | 26.1 | 47.0 | 18.0 | 27.0 | 30.6 | 24.8 | 39.8 | 46.0 | 13.8 |
| (Path) 1 | | | | | | | | | |
| Temporal | | | | | | | | | |
| Ctx | | | | | | | | | |
| Control | 24.5 | 25.9 | 18.4 | 16.0 | 20.4 | 9.8 | 24.8 | 0.0 | 2.6 |
| (Path) 2 | | | | | | | | | |
| Temporal | | | | | | | | | |
| Ctx | | | | | | | | | |
| Control | 11.7 | 16.0 | 5.6 | 7.5 | 10.9 | 3.5 | 11.9 | 31.0 | 6.3 |
| (Path) 3 | | | | | | | | | |
| Temporal | | | | | | | | | |
| Ctx | | | | | | | | | |
| Control | 21.9 | 27.4 | 16.8 | 17.1 | 18.2 | 14.8 | 21.6 | 39.5 | 7.0 |
| (Path) 4 | | | | | | | | | |
| Temporal | | | | | | | | | |
| Ctx | | | | | | | | | |
| AD 1 | 16.0 | 11.9 | 11.9 | 10.2 | 11.5 | 15.0 | 16.0 | 6.3 | 0.0 |
| Occipital | | | | | | | | | |
| Ctx | | | | | | | | | |
| AD 2 | 0.0 | 0.0 | 0.0 | 0.0 | 0.0 | 0.0 | 0.0 | 0.0 | 0.0 |
| Occipital | | | | | | | | | |
| Ctx | | | | | | | | | |
| (Missing) | | | | | | | | | |
| AD 3 | 10.7 | 6.0 | 8.3 | 6.4 | 8.8 | 8.0 | 10.2 | 4.9 | 0.0 |
| Occipital | | | | | | | | | |
| Ctx | | | | | | | | | |
| AD 4 | 18.9 | 23.7 | 5.8 | 13.0 | 17.9 | 6.8 | 18.6 | 11.1 | 3.5 |
| Occipital | | | | | | | | | |
| Ctx | | | | | | | | | |
| AD 5 | 24.8 | 28.3 | 25.2 | 25.3 | 22.5 | 12.7 | 22.7 | 42.3 | 3.8 |
| Occipital | | | | | | | | | |
| Ctx | | | | | | | | | |
| AD 6 | 20.6 | 31.9 | 19.8 | 20.2 | 17.0 | 5.9 | 22.1 | 14.8 | 8.5 |
| Occipital | | | | | | | | | |
| Ctx | | | | | | | | | |
| Control 1 | 9.5 | 14.4 | 6.6 | 6.0 | 8.7 | 4.1 | 7.2 | 8.8 | 1.3 |
| Occipital | | | | | | | | | |
| Ctx | | | | | | | | | |
| Control 2 | 31.9 | 42.6 | 15.7 | 26.4 | 33.2 | 20.3 | 29.3 | 82.4 | 13.7 |
| Occipital | | | | | | | | | |
| Ctx | | | | | | | | | |
| Control 3 | 18.8 | 13.0 | 5.7 | 10.7 | 17.1 | 7.5 | 19.2 | 8.8 | 5.0 |
| Occipital | | | | | | | | | |
| Ctx | | | | | | | | | |
| Control 4 | 18.2 | 17.0 | 21.6 | 12.0 | 12.6 | 3.3 | 13.6 | 24.0 | 1.3 |
| Occipital | | | | | | | | | |
| Ctx | | | | | | | | | |
| Control | 38.2 | 52.5 | 28.3 | 35.6 | 36.1 | 25.9 | 39.5 | 100.0 | 12.1 |
| (Path) 1 | | | | | | | | | |
| Occipital | | | | | | | | | |
| Ctx | | | | | | | | | |
| Control | 9.6 | 14.1 | 49.7 | 6.7 | 7.9 | 7.4 | 7.0 | 9.3 | 13.2 |
| (Path) 2 | | | | | | | | | |
| Occipital | | | | | | | | | |
| Ctx | | | | | | | | | |
| Control | 4.8 | 8.7 | 0.0 | 5.4 | 6.0 | 2.3 | 5.9 | 4.1 | 9.4 |
| (Path) 3 | | | | | | | | | |
| Occipital | | | | | | | | | |
| Ctx | | | | | | | | | |
| Control | 16.2 | 13.2 | 6.6 | 13.2 | 10.2 | 21.0 | 11.4 | 32.8 | 20.4 |
| (Path) 4 | | | | | | | | | |
| Occipital | | | | | | | | | |
| Ctx | | | | | | | | | |
| Control 1 | 14.4 | 21.9 | 8.8 | 8.8 | 16.3 | 12.5 | 15.7 | 9.2 | 5.0 |
| Parietal | | | | | | | | | |
| Ctx | | | | | | | | | |
| Control 2 | 32.8 | 28.9 | 14.5 | 34.4 | 28.3 | 41.2 | 37.1 | 28.1 | 25.5 |
| Parietal | | | | | | | | | |
| Ctx | | | | | | | | | |
| Control 3 | 20.6 | 19.8 | 19.9 | 11.5 | 8.7 | 13.2 | 10.8 | 9.1 | 16.7 |
| Parietal | | | | | | | | | |
| Ctx | | | | | | | | | |
| Control | 35.4 | 62.4 | 37.6 | 34.2 | 39.2 | 22.5 | 37.9 | 69.3 | 4.2 |
| (Path) 1 | | | | | | | | | |
| Parietal | | | | | | | | | |
| Ctx | | | | | | | | | |
| Control | 22.1 | 23.8 | 16.6 | 19.6 | 22.5 | 26.8 | 18.7 | 37.6 | 14.4 |
| (Path) 2 | | | | | | | | | |
| Parietal | | | | | | | | | |
| Ctx | | | | | | | | | |
| Control | 11.2 | 15.4 | 0.0 | 3.9 | 7.1 | 7.5 | 12.0 | 10.4 | 5.9 |
| (Path) 3 | | | | | | | | | |
| Parietal | | | | | | | | | |
| Ctx | | | | | | | | | |
| Control | 31.2 | 34.2 | 18.2 | 24.8 | 8.8 | 20.6 | 27.9 | 27.5 | 9.4 |
| (Path) 4 | | | | | | | | | |
| Parietal | | | | | | | | | |
| Ctx | | | | | | | | | |

TABLE AYL
General_screening_panel_v1.4

| | Rel. Exp. (%) |
|---|---|
| | Ag4983, Run |
| Tissue Name | 218328386 |
| Adipose | 25.3 |
| Melanoma* Hs688(A).T | 1.0 |
| Melanoma* Hs688(B).T | 2.9 |
| Melanoma* M14 | 0.7 |
| Melanoma* LOXIMVI | 0.0 |
| Melanoma* SK-MEL-5 | 29.9 |
| Squamous cell carcinoma SCC-4 | 0.1 |
| Testis Pool | 10.7 |
| Prostate ca.* (bone met) PC-3 | 2.9 |
| Prostate Pool | 18.4 |
| Placenta | 0.4 |
| Uterus Pool | 10.4 |
| Ovarian ca. OVCAR-3 | 1.2 |
| Ovarian ca. SK-OV-3 | 1.7 |
| Ovarian ca. OVCAR-4 | 0.6 |
| Ovarian ca. OVCAR-5 | 2.1 |
| Ovarian ca. IGROV-1 | 87.7 |
| Ovarian ca. OVCAR-8 | 10.6 |
| Ovary | 4.7 |
| Breast ca. MCF-7 | 0.4 |
| Breast ca. MDA-MB-231 | 0.4 |
| Breast ca. BT 549 | 0.6 |
| Breast ca. T47D | 5.1 |
| Breast ca. MDA-N | 1.0 |
| Breast Pool | 18.2 |
| Trachea | 8.9 |
| Lung | 3.7 |
| Fetal Lung | 7.2 |
| Lung ca. NCI-N417 | 2.3 |
| Lung ca. LX-1 | 9.7 |
| Lung ca. NCI-H146 | 0.3 |
| Lung ca. SHP-77 | 8.1 |
| Lung ca. A549 | 0.7 |
| Lung ca. NCI-H526 | 0.4 |
| Lung ca. NCI-H23 | 6.4 |
| Lung ca. NCI-H460 | 0.2 |
| Lung ca. HOP-62 | 0.9 |
| Lung ca. NCI-H522 | 2.2 |
| Liver | 0.2 |
| Fetal Liver | 0.6 |
| Liver ca. HepG2 | 0.3 |
| Kidney Pool | 41.8 |
| Fetal Kidney | 4.9 |
| Renal ca. 786-0 | 0.3 |
| Renal ca. A498 | 0.4 |
| Renal ca. ACHN | 2.1 |
| Renal ca. UO-31 | 0.6 |
| Renal ca. TK-10 | 3.0 |
| Bladder | 7.0 |
| Gastric ca. (liver met.) NCI-N87 | 1.9 |
| Gastric ca. KATO III | 0.7 |
| Colon ca. SW-948 | 0.1 |
| Colon ca. SW480 | 45.4 |
| Colon ca.* (SW480 met) SW620 | 17.1 |
| Colon ca. HT29 | 0.5 |
| Colon ca. HCT-116 | 5.3 |
| Colon ca. CaCo-2 | 21.8 |
| Colon cancer tissue | 12.7 |
| Colon ca. SW1116 | 2.4 |
| Colon ca. Colo-205 | 0.4 |
| Colon ca. SW-48 | 1.5 |
| Colon Pool | 31.4 |
| Small Intestine Pool | 12.1 |
| Stomach Pool | 13.6 |
| Bone Marrow Pool | 13.2 |
| Fetal Heart | 24.1 |
| Heart Pool | 34.9 |
| Lymph Node Pool | 26.4 |
| Fetal Skeletal Muscle | 55.1 |
| Skeletal Muscle Pool | 82.4 |
| Spleen Pool | 3.3 |
| Thymus Pool | 10.2 |
| CNS cancer (glio/astro) U87-MG | 14.9 |
| CNS cancer (glio/astro) U-118-MG | 5.1 |
| CNS cancer (neuro; met)SK-N-AS | 2.6 |
| CNS cancer (astro) SF-539 | 0.2 |
| CNS cancer (astro) SNB-75 | 11.9 |
| CNS cancer (glio) SNB-19 | 100.0 |
| CNS cancer (glio) SF-295 | 14.6 |
| Brain (Amygdala) Pool | 8.0 |
| Brain (cerebellum) | 11.5 |
| Brain (fetal) | 10.8 |
| Brain (Hippocampus) Pool | 11.6 |
| Cerebral Cortex Pool | 12.9 |
| Brain (Substantia nigra) Pool | 15.9 |
| Brain (Thalamus) Pool | 13.7 |
| Brain (whole) | 7.7 |
| Spinal Cord Pool | 14.9 |
| Adrenal Gland | 7.9 |
| Pituitary gland Pool | 1.3 |
| Salivary Gland | 1.6 |
| Thyroid (female) | 3.0 |
| Pancreatic ca. CAPAN2 | 1.5 |
| Pancreas Pool | 16.0 |

TABLE AYM
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.)NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro; met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE AYN
General_screening_panel_v1.6

| | Rel. Exp. | Rel. Exp. | Rel. Exp. | Rel. Exp. | Rel. Exp. | Rel. Exp. | Rel. Exp. | Rel. Exp. |
|---|---|---|---|---|---|---|---|---|
| | (%) Ag6413, | (%) Ag6425, | (%) Ag6428, | (%) Ag6431, | (%) Ag6431, | (%) Ag6435, | (%) Ag6439, | (%) Ag6964, |
| | Run | Run | Run | Run | Run | Run | Run | Run |
| Tissue Name | 277249371 | 277221721 | 277222439 | 277633568 | 278389390 | 277223167 | 277223175 | 278388946 |
| Adipose | 25.9 | 2.6 | 20.0 | 17.4 | 13.8 | 13.2 | 17.3 | 18.8 |
| Melanoma* | 0.5 | 0.0 | 2.0 | 0.8 | 0.9 | 0.9 | 0.4 | 0.7 |
| Hs688(A).T | | | | | | | | |
| Melanoma* | 2.7 | 0.2 | 4.1 | 2.5 | 2.2 | 1.9 | 2.9 | 2.4 |
| Hs688(B).T | | | | | | | | |
| Melanoma* | 0.3 | 0.0 | 0.7 | 0.4 | 0.4 | 0.0 | 0.4 | 0.7 |
| M14 | | | | | | | | |
| Melanoma* | 0.0 | 0.0 | 0.1 | 0.0 | 0.0 | 0.0 | 0.0 | 0.1 |
| LOXIMVI | | | | | | | | |
| Melanoma* | 15.2 | 2.2 | 30.4 | 18.2 | 14.6 | 4.4 | 18.3 | 15.9 |
| SK-MEL-5 | | | | | | | | |
| Squamous cell | 0.0 | 0.0 | 0.1 | 0.1 | 0.2 | 0.0 | 0.0 | 0.1 |
| carcinoma | | | | | | | | |
| SCC-4 | | | | | | | | |
| Testis Pool | 5.2 | 3.5 | 8.8 | 10.4 | 9.0 | 10.0 | 9.1 | 9.9 |
| Prostate ca.* | 1.9 | 0.5 | 2.5 | 1.9 | 1.8 | 1.8 | 1.3 | 4.3 |
| (bone met) | | | | | | | | |
| PC-3 | | | | | | | | |
| Prostate Pool | 8.1 | 1.0 | 11.5 | 11.3 | 12.1 | 10.0 | 28.5 | 10.0 |
| Placenta | 0.5 | 0.0 | 0.7 | 0.1 | 0.1 | 0.3 | 0.5 | 0.4 |
| Uterus Pool | 2.2 | 1.5 | 4.5 | 4.6 | 4.5 | 16.2 | 5.3 | 4.1 |
| Ovarian ca. | 0.9 | 0.3 | 1.1 | 0.7 | 1.1 | 0.4 | 1.6 | 4.0 |
| OVCAR-3 | | | | | | | | |
| Ovarian ca. | 0.8 | 0.2 | 1.7 | 0.8 | 0.9 | 0.9 | 1.3 | 1.7 |
| SK-OV-3 | | | | | | | | |
| Ovarian ca. | 0.2 | 0.0 | 0.9 | 0.4 | 0.8 | 0.0 | 0.9 | 0.5 |
| OVCAR-4 | | | | | | | | |
| Ovarian ca. | 1.6 | 1.3 | 2.9 | 1.3 | 1.7 | 0.3 | 1.4 | 7.9 |
| OVCAR-5 | | | | | | | | |
| Ovarian ca. | 100.0 | 100.0 | 77.9 | 84.7 | 97.9 | 27.0 | 69.3 | 75.8 |
| IGROV-1 | | | | | | | | |
| Ovarian ca. | 13.6 | 21.9 | 14.0 | 15.6 | 14.6 | 7.6 | 17.3 | 16.7 |
| OVCAR-8 | | | | | | | | |
| Ovary | 2.7 | 0.3 | 5.2 | 3.1 | 2.3 | 4.5 | 2.8 | 2.4 |
| Breast ca. | 0.3 | 0.0 | 0.3 | 0.1 | 0.2 | 0.0 | 0.5 | 0.5 |
| MCF-7 | | | | | | | | |
| Breast ca. | 0.1 | 0.0 | 0.4 | 0.2 | 0.2 | 0.0 | 0.2 | 0.3 |
| MDA-MB- | | | | | | | | |
| 231 | | | | | | | | |
| Breast ca. | 0.5 | 0.0 | 0.5 | 0.1 | 0.5 | 0.0 | 0.6 | 0.4 |
| BT 549 | | | | | | | | |
| Breast ca. | 0.0 | 0.0 | 0.5 | 0.2 | 0.3 | 0.0 | 0.4 | 0.5 |
| T47D | | | | | | | | |
| Breast ca. | 0.6 | 0.0 | 0.7 | 0.6 | 0.6 | 0.7 | 0.6 | 0.8 |
| MDA-N | | | | | | | | |
| Breast Pool | 15.0 | 4.1 | 21.8 | 14.6 | 10.7 | 42.9 | 12.2 | 16.7 |
| Trachea | 4.5 | 0.7 | 8.4 | 4.8 | 4.2 | 8.3 | 4.7 | 5.6 |
| Lung | 2.8 | 0.7 | 2.3 | 4.2 | 3.2 | 3.9 | 3.9 | 5.1 |
| Fetal Lung | 3.9 | 0.3 | 9.1 | 5.0 | 4.8 | 8.0 | 5.3 | 6.1 |
| Lung ca. NCI- | 2.0 | 0.9 | 3.5 | 3.3 | 2.6 | 0.2 | 4.0 | 2.3 |
| N417 | | | | | | | | |
| Lung ca. LX-1 | 3.5 | 2.7 | 6.5 | 5.0 | 3.5 | 0.9 | 4.9 | 44.1 |
| Lung ca. NCI- | 0.1 | 0.0 | 0.3 | 0.1 | 0.2 | 0.0 | 0.1 | 0.1 |
| H146 | | | | | | | | |
| Lung ca. SHP- | 4.0 | 0.4 | 6.8 | 5.3 | 4.5 | 0.2 | 4.5 | 3.8 |
| 77 | | | | | | | | |
| Lung ca. | 0.3 | 2.6 | 0.9 | 0.0 | 0.4 | 0.0 | 0.6 | 4.7 |
| A549 | | | | | | | | |
| Lung ca. NCI- | 0.2 | 0.0 | 0.9 | 0.6 | 0.3 | 0.0 | 0.4 | 0.5 |
| H526 | | | | | | | | |
| Lung ca. NCI- | 2.9 | 1.0 | 4.6 | 4.8 | 3.2 | 0.6 | 2.9 | 10.3 |
| H23 | | | | | | | | |
| Lung ca. NCI- | 0.0 | 0.0 | 0.2 | 0.1 | 0.3 | 0.0 | 0.0 | 0.3 |
| H460 | | | | | | | | |
| Lung ca. | 0.5 | 0.0 | 0.5 | 1.0 | 0.6 | 0.0 | 0.5 | 0.7 |
| HOP-62 | | | | | | | | |
| Lung ca. NCI- | 1.7 | 0.6 | 2.3 | 1.7 | 1.3 | 0.0 | 3.3 | 8.9 |
| H522 | | | | | | | | |
| Liver | 0.1 | 0.0 | 0.0 | 0.0 | 0.0 | 0.0 | 0.1 | 2.0 |
| Fetal Liver | 0.3 | 0.3 | 1.1 | 0.6 | 0.5 | 0.3 | 0.8 | 8.2 |
| Liver ca. | 0.1 | 0.3 | 0.2 | 0.0 | 0.2 | 0.0 | 0.1 | 2.4 |
| HepG2 | | | | | | | | |
| Kidney Pool | 27.9 | 0.0 | 47.0 | 33.9 | 28.1 | 100.0 | 43.2 | 32.8 |
| Fetal Kidney | 1.4 | 0.0 | 4.9 | 4.1 | 4.0 | 12.1 | 5.8 | 11.5 |
| Renal ca. | 0.2 | 0.0 | 0.2 | 0.3 | 0.1 | 0.0 | 0.3 | 0.9 |
| 786-0 | | | | | | | | |
| Renal ca. | 0.0 | 1.8 | 0.2 | 0.0 | 0.3 | 0.0 | 0.5 | 8.5 |
| A498 | | | | | | | | |
| Renal ca. | 1.5 | 0.5 | 2.5 | 1.7 | 1.5 | 0.0 | 1.2 | 2.5 |
| ACHN | | | | | | | | |
| Renal ca. | 0.3 | 0.0 | 0.5 | 0.2 | 0.2 | 0.0 | 0.6 | 0.3 |
| UO-31 | | | | | | | | |
| Renal ca. | 1.9 | 0.4 | 3.1 | 2.0 | 1.9 | 0.7 | 2.1 | 4.6 |
| TK-10 | | | | | | | | |
| Bladder | 4.2 | 0.0 | 5.9 | 5.5 | 5.1 | 6.6 | 8.3 | 6.7 |
| Gastric ca. | 0.9 | 0.0 | 1.7 | 0.9 | 1.2 | 0.0 | 1.1 | 6.7 |
| (liver met.) | | | | | | | | |
| NCI-N87 | | | | | | | | |
| Gastric ca. | 0.4 | 0.5 | 0.8 | 0.2 | 0.3 | 0.3 | 0.4 | 0.9 |
| KATO III | | | | | | | | |
| Colon ca. | 0.0 | 1.5 | 0.2 | 0.2 | 0.2 | 0.0 | 0.3 | 1.2 |
| SW-948 | | | | | | | | |
| Colon ca. | 20.9 | 5.2 | 41.8 | 27.0 | 23.3 | 4.4 | 23.0 | 33.7 |
| SW480 | | | | | | | | |
| Colon ca.* | 13.3 | 4.8 | 16.4 | 12.8 | 10.3 | 1.7 | 6.1 | 25.0 |
| (SW480 met) | | | | | | | | |
| SW620 | | | | | | | | |
| Colon ca. | 0.2 | 0.0 | 0.0 | 0.2 | 0.2 | 0.0 | 0.0 | 0.3 |
| HT29 | | | | | | | | |
| Colon ca. | 2.1 | 0.2 | 3.2 | 2.5 | 2.0 | 0.5 | 2.1 | 4.3 |
| HCT-116 | | | | | | | | |
| Colon ca. | 5.0 | 3.6 | 27.0 | 19.1 | 16.7 | 7.6 | 18.3 | 38.2 |
| CaCo-2 | | | | | | | | |
| Colon cancer | 9.0 | 3.3 | 11.0 | 11.9 | 7.6 | 5.6 | 7.7 | 20.4 |
| tissue | | | | | | | | |
| Colon ca. | 1.3 | 3.0 | 2.5 | 2.0 | 1.5 | 1.1 | 1.8 | 6.0 |
| SW1116 | | | | | | | | |
| Colon ca. | 0.1 | 0.4 | 0.3 | 0.2 | 0.0 | 0.0 | 0.2 | 0.8 |
| Colo-205 | | | | | | | | |
| Colon ca. | 0.8 | 3.6 | 1.4 | 1.5 | 1.5 | 0.0 | 1.4 | 2.6 |
| SW-48 | | | | | | | | |
| Colon Pool | 20.3 | 5.0 | 28.1 | 23.2 | 18.7 | 44.8 | 25.5 | 20.6 |
| Small | 14.0 | 1.7 | 17.1 | 11.2 | 13.0 | 26.8 | 12.8 | 10.4 |
| Intestine Pool | | | | | | | | |
| Stomach Pool | 8.1 | 2.3 | 14.3 | 9.5 | 9.3 | 24.0 | 8.5 | 10.7 |
| Bone Marrow | 6.8 | 1.6 | 14.3 | 10.2 | 8.7 | 25.9 | 18.7 | 12.5 |
| Pool | | | | | | | | |
| Fetal Heart | 10.1 | 2.3 | 25.5 | 24.5 | 21.8 | 31.6 | 33.7 | 20.7 |
| Heart Pool | 28.7 | 7.0 | 29.7 | 25.9 | 17.2 | 23.5 | 33.7 | 26.1 |
| Lymph Node | 17.6 | 6.1 | 33.7 | 22.1 | 23.7 | 64.6 | 19.9 | 24.7 |
| Pool | | | | | | | | |
| Fetal Skeletal | 31.9 | 5.2 | 54.3 | 48.6 | 46.3 | 46.7 | 19.1 | 50.7 |
| Muscle | | | | | | | | |
| Skeletal | 17.4 | 9.2 | 29.3 | 29.5 | 25.9 | 24.7 | 22.1 | 32.3 |
| Muscle Pool | | | | | | | | |
| Spleen Pool | 0.9 | 0.0 | 1.9 | 2.0 | 1.7 | 2.4 | 2.7 | 3.1 |
| Thymus Pool | 4.4 | 2.0 | 10.4 | 8.1 | 9.4 | 18.4 | 7.7 | 7.0 |
| CNS cancer | 9.8 | 1.5 | 14.9 | 10.7 | 10.0 | 5.8 | 10.9 | 14.1 |
| (glio/astro) | | | | | | | | |
| U87-MG | | | | | | | | |
| CNS cancer | 3.5 | 0.3 | 4.7 | 3.8 | 3.1 | 1.5 | 3.8 | 5.8 |
| (glio/astro) | | | | | | | | |
| U-118-MG | | | | | | | | |
| CNS cancer | 1.9 | 0.0 | 2.6 | 2.1 | 1.0 | 0.7 | 1.4 | 2.6 |
| (neuro; met) | | | | | | | | |
| SK-N-AS | | | | | | | | |
| CNS cancer | 0.1 | 0.0 | 0.0 | 0.1 | 0.2 | 0.2 | 0.1 | 0.1 |
| (astro) SF-539 | | | | | | | | |
| CNS cancer | 8.1 | 1.1 | 14.9 | 6.5 | 10.0 | 3.1 | 11.7 | 9.7 |
| (astro) SNB- | | | | | | | | |
| 75 | | | | | | | | |
| CNS cancer | 79.6 | 79.0 | 100.0 | 100.0 | 100.0 | 12.8 | 100.0 | 100.0 |
| (glio) SNB-19 | | | | | | | | |
| CNS cancer | 8.2 | 0.0 | 11.3 | 8.0 | 7.8 | 0.0 | 8.2 | 14.8 |
| (glio) SF-295 | | | | | | | | |
| Brain | 3.7 | 0.8 | 7.7 | 6.2 | 4.8 | 7.9 | 8.0 | 5.3 |
| (Amygdala) | | | | | | | | |
| Pool | | | | | | | | |
| Brain | 12.0 | 0.4 | 19.8 | 10.7 | 9.7 | 1.8 | 8.8 | 9.7 |
| (cerebellum) | | | | | | | | |
| Brain (fetal) | 4.2 | 0.7 | 12.7 | 6.6 | 5.6 | 8.4 | 6.8 | 6.4 |
| Brain | 7.5 | 3.2 | 11.7 | 8.6 | 6.9 | 9.9 | 11.0 | 10.2 |
| (Hippocampus) | | | | | | | | |
| Pool | | | | | | | | |
| Cerebral | 9.7 | 0.6 | 11.0 | 7.5 | 0.7 | 1.8 | 11.6 | 8.7 |
| Cortex Pool | | | | | | | | |
| Brain | 7.4 | 2.2 | 11.7 | 10.4 | 4.7 | 4.2 | 10.0 | 9.3 |
| (Substantia | | | | | | | | |
| nigra) Pool | | | | | | | | |
| Brain | 7.6 | 2.7 | 13.2 | 9.3 | 0.2 | 9.1 | 9.7 | 8.7 |
| (Thalamus) | | | | | | | | |
| Pool | | | | | | | | |
| Brain (whole) | 6.1 | 0.4 | 10.6 | 5.8 | 0.3 | 3.3 | 5.6 | 8.7 |
| Spinal Cord | 10.1 | 2.3 | 14.7 | 11.0 | 7.6 | 13.1 | 12.2 | 9.0 |
| Pool | | | | | | | | |
| Adrenal Gland | 3.5 | 0.3 | 9.9 | 3.9 | 3.7 | 7.4 | 4.8 | 4.1 |
| Pituitary gland | 0.9 | 0.0 | 1.1 | 1.2 | 1.1 | 1.8 | 1.4 | 0.5 |
| Pool | | | | | | | | |
| Salivary | 0.9 | 0.0 | 1.8 | 1.3 | 0.9 | 2.3 | 1.1 | 1.0 |
| Gland | | | | | | | | |
| Thyroid | 2.0 | 0.3 | 3.1 | 2.5 | 2.5 | 3.3 | 1.9 | 2.3 |
| (female) | | | | | | | | |
| Pancreatic ca. | 0.5 | 0.0 | 0.8 | 0.7 | 0.6 | 0.5 | 0.7 | 2.2 |
| CAPAN2 | | | | | | | | |
| Pancreas Pool | 1.2 | 0.0 | 2.0 | 1.1 | 1.6 | 3.5 | 3.2 | 2.3 |

TABLE AYO
Panel 4.1D

| | Rel. Exp. | Rel. Exp. | Rel. Exp. | Rel. Exp. | Rel. Exp. | Rel. Exp. | Rel. Exp. | Rel. Exp. |
|---|---|---|---|---|---|---|---|---|
| | (%) Ag4983, | (%) Ag6413, | (%) Ag6425, | (%) Ag6428, | (%) Ag6431, | (%) Ag6435, | (%) Ag6439, | (%) Ag6447, |
| | Run | Run | Run | Run | Run | Run | Run | Run |
| Tissue Name | 218623570 | 269239947 | 268713999 | 268767535 | 268767577 | 268713480 | 268760823 | 268761806 |
| Secondary Th1 | 0.1 | 0.3 | 0.0 | 1.3 | 0.7 | 0.0 | 0.0 | 0.0 |
| act | | | | | | | | |
| Secondary Th2 | 0.5 | 0.3 | 0.0 | 1.2 | 0.8 | 0.0 | 0.0 | 0.0 |
| act | | | | | | | | |
| Secondary Tr1 | 0.0 | 0.0 | 0.0 | 0.0 | 0.7 | 0.0 | 0.0 | 0.0 |
| act | | | | | | | | |
| Secondary Th1 | 0.1 | 0.0 | 0.0 | 0.0 | 0.0 | 0.0 | 0.0 | 0.0 |
| rest | | | | | | | | |
| Secondary Th2 | 0.3 | 0.0 | 0.0 | 0.0 | 0.0 | 0.7 | 0.0 | 0.0 |
| rest | | | | | | | | |
| Secondary Tr1 | 0.1 | 0.3 | 0.0 | 0.4 | 0.0 | 0.0 | 0.0 | 0.0 |
| rest | | | | | | | | |
| Primary Th1 act | 0.0 | 0.0 | 0.0 | 0.0 | 0.0 | 0.0 | 0.0 | 0.0 |
| Primary Th2 act | 0.2 | 0.4 | 0.0 | 0.3 | 0.4 | 0.7 | 0.0 | 0.0 |
| Primary Tr1 act | 0.1 | 0.0 | 0.0 | 0.7 | 0.7 | 0.0 | 0.0 | 0.0 |
| Primary Th1 | 0.0 | 0.0 | 0.0 | 0.1 | 0.3 | 0.0 | 1.2 | 0.0 |
| rest | | | | | | | | |
| Primary Th2 | 0.0 | 0.0 | 0.0 | 0.4 | 0.2 | 0.0 | 0.0 | 0.0 |
| rest | | | | | | | | |
| Primary Tr1 | 0.3 | 0.0 | 0.0 | 0.0 | 0.0 | 0.0 | 0.0 | 0.0 |
| rest | | | | | | | | |
| CD45RA CD4 | 0.4 | 2.8 | 0.0 | 5.4 | 2.4 | 0.8 | 2.6 | 0.0 |
| lymphocyte act | | | | | | | | |
| CD45RO CD4 | 0.1 | 2.2 | 0.0 | 1.5 | 0.7 | 1.6 | 2.3 | 0.0 |
| lymphocyte act | | | | | | | | |
| CD8 | 0.4 | 0.9 | 0.0 | 0.7 | 0.0 | 0.0 | 0.0 | 0.0 |
| lymphocyte act | | | | | | | | |
| Secondary CD8 | 0.1 | 0.0 | 0.0 | 8.8 | 0.0 | 0.0 | 0.0 | 0.0 |
| lymphocyte rest | | | | | | | | |
| Secondary CD8 | 0.0 | 0.1 | 0.0 | 0.4 | 0.3 | 0.0 | 0.0 | 0.0 |
| lymphocyte act | | | | | | | | |
| CD4 lymphocyte | 0.1 | 0.0 | 0.0 | 0.5 | 0.4 | 0.0 | 0.0 | 0.0 |
| none | | | | | | | | |
| 2ry | 0.3 | 0.2 | 0.0 | 0.0 | 0.0 | 0.0 | 1.2 | 0.0 |
| Th1/Th2/Tr1_{—} | | | | | | | | |
| anti-CD95 CH11 | | | | | | | | |
| LAK cells rest | 5.6 | 5.0 | 2.7 | 11.8 | 3.8 | 6.1 | 15.2 | 0.0 |
| LAK cells IL-2 | 0.4 | 0.3 | 0.0 | 0.0 | 0.0 | 0.0 | 0.0 | 0.0 |
| LAK cells | 0.2 | 0.0 | 0.0 | 0.0 | 0.0 | 0.0 | 0.0 | 0.0 |
| IL-2 + IL-12 | | | | | | | | |
| LAK cells | 0.1 | 0.3 | 0.0 | 0.0 | 0.0 | 0.0 | 0.0 | 0.0 |
| IL-2 + IFN gamma | | | | | | | | |
| LAK cells IL-2 + | 0.0 | 0.0 | 0.0 | 0.0 | 0.0 | 0.0 | 0.0 | 0.0 |
| IL-18 | | | | | | | | |
| LAK cells | 4.5 | 4.0 | 15.7 | 15.1 | 6.3 | 6.1 | 9.0 | 0.0 |
| PMA/ionomycin | | | | | | | | |
| NK Cells IL-2 | 0.9 | 0.1 | 0.0 | 3.4 | 2.5 | 0.0 | 1.4 | 0.0 |
| rest | | | | | | | | |
| Two Way MLR | 1.4 | 1.1 | 0.0 | 2.2 | 1.3 | 0.9 | 1.4 | 0.0 |
| 3 day | | | | | | | | |
| Two Way MLR | 4.5 | 0.9 | 0.0 | 0.8 | 0.9 | 0.0 | 0.0 | 0.0 |
| 5 day | | | | | | | | |
| Two Way MLR | 2.3 | 0.7 | 13.2 | 1.1 | 2.6 | 2.9 | 3.7 | 0.0 |
| 7 day | | | | | | | | |
| PBMC rest | 0.1 | 0.0 | 0.0 | 0.0 | 0.0 | 0.0 | 0.0 | 0.0 |
| PBMC PWM | 0.6 | 0.0 | 0.0 | 1.3 | 0.0 | 0.0 | 0.0 | 0.0 |
| PBMC PHA-L | 0.3 | 0.2 | 0.0 | 0.6 | 0.7 | 0.0 | 0.0 | 0.0 |
| Ramos (B cell) | 0.1 | 0.0 | 0.0 | 0.0 | 0.0 | 0.0 | 0.0 | 0.0 |
| none | | | | | | | | |
| Ramos (B cell) | 0.0 | 0.0 | 0.0 | 0.7 | 0.2 | 0.0 | 0.0 | 0.0 |
| ionomycin | | | | | | | | |
| B lymphocytes | 0.5 | 0.0 | 0.0 | 0.0 | 0.0 | 0.0 | 0.0 | 0.0 |
| PWM | | | | | | | | |
| B lymphocytes | 0.2 | 0.0 | 0.0 | 0.9 | 0.0 | 0.0 | 0.0 | 0.0 |
| CD40L and IL-4 | | | | | | | | |
| EOL-1 | 3.7 | 2.6 | 9.1 | 29.1 | 8.1 | 0.0 | 68.8 | 0.0 |
| dbcAMP | | | | | | | | |
| EOL-1 | 1.6 | 0.7 | 0.0 | 0.0 | 2.7 | 1.0 | 1.8 | 0.0 |
| dbcAMP | | | | | | | | |
| PMA/ionomycin | | | | | | | | |
| Dendritic cells | 5.6 | 3.1 | 13.8 | 4.1 | 5.3 | 0.7 | 0.0 | 0.0 |
| none | | | | | | | | |
| Dendritic cells | 1.6 | 0.3 | 0.0 | 1.0 | 0.7 | 0.0 | 0.0 | 0.0 |
| LPS | | | | | | | | |
| Dendritic cells | 2.0 | 1.6 | 3.3 | 0.5 | 0.2 | 1.6 | 0.0 | 0.0 |
| anti-CD40 | | | | | | | | |
| Monocytes rest | 0.2 | 0.0 | 0.0 | 0.4 | 0.0 | 0.0 | 0.0 | 0.0 |
| Monocytes LPS | 2.2 | 3.3 | 0.0 | 5.7 | 1.8 | 0.0 | 2.6 | 0.4 |
| Macrophages | 0.9 | 1.8 | 0.0 | 0.6 | 0.6 | 0.0 | 0.0 | 0.0 |
| rest | | | | | | | | |
| Macrophages | 7.5 | 4.0 | 0.0 | 5.4 | 6.3 | 0.8 | 9.2 | 0.0 |
| LPS | | | | | | | | |
| HUVEC none | 0.1 | 0.0 | 0.0 | 0.0 | 0.0 | 0.0 | 0.0 | 0.0 |
| HUVEC | 0.0 | 0.0 | 0.0 | 0.0 | 0.3 | 0.0 | 0.0 | 0.0 |
| starved | | | | | | | | |
| HUVEC IL- | 0.0 | 0.0 | 0.0 | 0.0 | 0.5 | 0.0 | 0.0 | 0.0 |
| 1beta | | | | | | | | |
| HUVEC IFN | 0.2 | 0.0 | 0.0 | 0.0 | 0.0 | 0.0 | 0.0 | 0.0 |
| gamma | | | | | | | | |
| HUVEC TNF | 0.0 | 0.0 | 0.0 | 0.0 | 0.0 | 0.6 | 0.0 | 0.0 |
| alpha + IFN | | | | | | | | |
| gamma | | | | | | | | |
| HUVEC TNF | 0.6 | 0.0 | 0.0 | 0.0 | 0.4 | 0.0 | 0.0 | 0.0 |
| alpha + IL4 | | | | | | | | |
| HUVEC IL-11 | 0.0 | 0.0 | 0.0 | 0.4 | 0.3 | 0.0 | 0.0 | 0.0 |
| Lung | 0.2 | 0.3 | 0.0 | 0.4 | 0.0 | 0.0 | 0.0 | 0.0 |
| Microvascular | | | | | | | | |
| EC none | | | | | | | | |
| Lung | 0.1 | 0.0 | 0.0 | 0.0 | 0.0 | 0.0 | 0.0 | 0.0 |
| Microvascular | | | | | | | | |
| EC TNFalpha + | | | | | | | | |
| IL-1beta | | | | | | | | |
| Microvascular | 0.1 | 0.0 | 0.0 | 0.0 | 0.0 | 0.0 | 0.0 | 0.0 |
| Dermal EC | | | | | | | | |
| none | | | | | | | | |
| Microsvasular | 0.1 | 0.0 | 0.0 | 0.0 | 0.0 | 0.0 | 0.0 | 0.0 |
| Dermal EC | | | | | | | | |
| TNFalpha + IL- | | | | | | | | |
| 1beta | | | | | | | | |
| Bronchial | 0.0 | 0.0 | 0.0 | 0.0 | 0.0 | 0.0 | 0.0 | 0.0 |
| epithelium | | | | | | | | |
| TNFalpha + | | | | | | | | |
| IL1beta | | | | | | | | |
| Small airway | 0.0 | 0.0 | 0.0 | 0.0 | 0.0 | 0.0 | 0.0 | 0.0 |
| epithelium none | | | | | | | | |
| Small airway | 0.3 | 0.0 | 0.0 | 0.0 | 0.0 | 0.0 | 0.0 | 0.0 |
| epithelium | | | | | | | | |
| TNFalpha + IL- | | | | | | | | |
| 1beta | | | | | | | | |
| Coronery artery | 0.1 | 0.6 | 0.0 | 0.0 | 0.0 | 0.5 | 0.0 | 0.3 |
| SMC rest | | | | | | | | |
| Coronery artery | 0.4 | 0.9 | 6.2 | 0.3 | 1.5 | 0.0 | 0.0 | 0.0 |
| SMC TNFalpha + | | | | | | | | |
| IL-1beta | | | | | | | | |
| Astrocytes rest | 67.8 | 97.3 | 100.0 | 100.0 | 100.0 | 100.0 | 100.0 | 54.3 |
| Astrocytes | 100.0 | 100.0 | 74.2 | 97.3 | 74.7 | 97.9 | 95.9 | 100.0 |
| TNFalpha + IL- | | | | | | | | |
| 1beta | | | | | | | | |
| KU-812 | 0.1 | 0.0 | 0.0 | 0.0 | 0.4 | 0.0 | 0.0 | 0.0 |
| (Basophil) rest | | | | | | | | |
| KU-812 | 0.0 | 0.0 | 0.0 | 0.0 | 0.0 | 0.0 | 0.0 | 0.0 |
| (Basophil) | | | | | | | | |
| PMA/ionomycin | | | | | | | | |
| CCD1106 | 0.2 | 0.0 | 0.0 | 0.0 | 0.8 | 0.0 | 0.0 | 0.0 |
| (Keratinocytes) | | | | | | | | |
| none | | | | | | | | |
| CCD1106 | 0.3 | 0.0 | 0.0 | 0.0 | 0.0 | 0.0 | 0.0 | 0.0 |
| (Keratinocytes) | | | | | | | | |
| TNFalpha + IL- | | | | | | | | |
| 1beta | | | | | | | | |
| Liver cirrhosis | 2.3 | 7.2 | 4.6 | 2.6 | 6.7 | 5.1 | 8.5 | 0.6 |
| NCI-H292 none | 0.3 | 0.3 | 0.0 | 1.7 | 0.6 | 0.0 | 0.0 | 0.0 |
| NCI-H292 IL-4 | 0.3 | 0.0 | 0.0 | 0.0 | 0.5 | 0.0 | 0.0 | 0.0 |
| NCI-H292 IL-9 | 0.3 | 0.0 | 0.0 | 0.7 | 0.5 | 0.0 | 0.0 | 0.0 |
| NCI-H292 IL-13 | 0.6 | 0.6 | 0.0 | 0.9 | 0.9 | 0.0 | 0.0 | 0.0 |
| NCI-H292 IFN | 0.2 | 0.0 | 0.0 | 0.5 | 0.6 | 0.0 | 0.0 | 0.0 |
| gamma | | | | | | | | |
| HPAEC none | 0.0 | 0.3 | 0.0 | 0.0 | 0.0 | 0.0 | 0.0 | 0.0 |
| HPAEC TNF | 0.0 | 0.3 | 0.0 | 0.0 | 0.0 | 0.0 | 0.0 | 0.0 |
| alpha + IL-1 | | | | | | | | |
| beta | | | | | | | | |
| Lung fibroblast | 29.7 | 62.9 | 31.4 | 95.9 | 65.5 | 62.9 | 94.0 | 26.2 |
| none | | | | | | | | |
| Lung fibroblast | 16.0 | 36.9 | 22.2 | 48.6 | 39.8 | 25.2 | 62.9 | 28.3 |
| TNF alpha + | | | | | | | | |
| IL-1 beta | | | | | | | | |
| Lung fibroblast | 26.1 | 28.7 | 19.1 | 27.4 | 21.2 | 23.3 | 34.9 | 16.0 |
| IL-4 | | | | | | | | |
| Lung fibroblast | 28.5 | 42.0 | 23.5 | 24.0 | 26.8 | 20.4 | 96.6 | 9.3 |
| IL-9 | | | | | | | | |
| Lung fibroblast | 31.6 | 14.6 | 4.5 | 11.9 | 10.4 | 15.0 | 13.4 | 4.3 |
| IL-13 | | | | | | | | |
| Lung fibroblast | 20.4 | 32.8 | 15.7 | 55.9 | 46.3 | 29.9 | 89.5 | 25.2 |
| IFN gamma | | | | | | | | |
| Dermal | 2.5 | 2.9 | 0.0 | 6.0 | 6.3 | 5.6 | 4.1 | 0.0 |
| fibroblast | | | | | | | | |
| CCD1070 rest | | | | | | | | |
| Dermal | 1.1 | 1.3 | 0.0 | 2.7 | 0.8 | 0.8 | 2.3 | 1.1 |
| fibroblast | | | | | | | | |
| CCD1070 TNF | | | | | | | | |
| alpha | | | | | | | | |
| Dermal | 1.9 | 2.9 | 0.0 | 5.6 | 1.3 | 0.7 | 0.0 | 1.6 |
| fibroblast | | | | | | | | |
| CCD1070 IL-1 | | | | | | | | |
| beta | | | | | | | | |
| Dermal | 9.3 | 20.3 | 8.5 | 30.6 | 20.2 | 20.0 | 26.6 | 4.9 |
| fibroblast IFN | | | | | | | | |
| gamma | | | | | | | | |
| Dermal | 10.7 | 14.6 | 4.1 | 30.8 | 19.8 | 22.7 | 25.5 | 13.5 |
| fibroblast IL-4 | | | | | | | | |
| Dermal | 24.8 | 42.3 | 8.0 | 54.3 | 46.7 | 20.7 | 47.3 | 15.8 |
| Fibroblasts rest | | | | | | | | |
| Neutrophils | 0.7 | 0.0 | 0.0 | 0.9 | 0.4 | 1.2 | 0.0 | 0.0 |
| TNFa + LPS | | | | | | | | |
| Neutrophils rest | 0.1 | 0.0 | 0.0 | 0.0 | 0.3 | 0.0 | 0.0 | 0.0 |
| Colon | 7.9 | 4.7 | 4.0 | 4.6 | 9.5 | 7.9 | 8.4 | 4.8 |
| Lung | 2.2 | 1.2 | 0.0 | 2.8 | 4.6 | 1.6 | 2.1 | 0.0 |
| Thymus | 3.1 | 0.8 | 0.0 | 0.0 | 0.4 | 2.0 | 2.4 | 0.0 |
| Kidney | 4.2 | 4.4 | 4.9 | 7.8 | 9.7 | 10.2 | 5.2 | 0.6 |

TABLE AYP
general oncology screening panel_v_2.4

| | Rel. Exp. (%) | Rel. Exp. (%) |
|---|---|---|
| | Ag4983, Run | Ag6442, Run |
| Tissue Name | 260281959 | 264979180 |
| Colon cancer 1 | 12.1 | 22.7 |
| Colon NAT 1 | 100.0 | 100.0 |
| Colon cancer 2 | 6.5 | 0.0 |
| Colon NAT 2 | 8.0 | 15.1 |
| Colon cancer 3 | 7.4 | 2.8 |
| Colon NAT 3 | 39.8 | 40.1 |
| Colon malignant cancer 4 | 15.0 | 9.5 |
| Colon NAT 4 | 3.5 | 0.9 |
| Lung cancer 1 | 1.4 | 6.6 |
| Lung NAT 1 | 0.6 | 0.0 |
| Lung cancer 2 | 26.6 | 15.9 |
| Lung NAT 2 | 2.7 | 0.0 |
| Squamous cell carcinoma 3 | 5.6 | 8.3 |
| Lung NAT 3 | 0.8 | 0.0 |
| Metastatic melanoma 1 | 27.2 | 49.0 |
| Melanoma 2 | 2.5 | 1.1 |
| Melanoma 3 | 2.3 | 13.8 |
| Metastatic melanoma 4 | 33.9 | 24.0 |
| Metastatic melanoma 5 | 34.6 | 31.4 |
| Bladder cancer 1 | 1.3 | 2.1 |
| Bladder NAT 1 | 0.0 | 0.0 |
| Bladder cancer 2 | 8.7 | 19.3 |
| Bladder NAT 2 | 1.7 | 1.4 |
| Bladder NAT 3 | 0.2 | 4.8 |
| Bladder NAT 4 | 27.0 | 66.0 |
| Prostate adenocarcinoma 1 | 9.2 | 7.5 |
| Prostate adenocarcinoma 2 | 3.5 | 8.0 |
| Prostate adenocarcinoma 3 | 14.3 | 9.0 |
| Prostate adenocarcinoma 4 | 16.4 | 9.1 |
| Prostate NAT 5 | 16.8 | 9.9 |
| Prostate adenocarcinoma 6 | 3.2 | 7.7 |
| Prostate adenocarcinoma 7 | 9.2 | 17.3 |
| Prostate adenocarcinoma 8 | 3.0 | 0.0 |
| Prostate adenocarcinoma 9 | 27.0 | 33.9 |
| Prostate NAT 10 | 3.8 | 4.9 |
| Kidney cancer 1 | 24.0 | 16.5 |
| Kidney NAT 1 | 15.6 | 7.2 |
| Kidney cancer 2 | 91.4 | 73.7 |
| Kidney NAT 2 | 22.1 | 19.2 |
| Kidney cancer 3 | 27.0 | 21.3 |
| Kidney NAT 3 | 9.3 | 11.4 |
| Kidney cancer 4 | 20.0 | 25.7 |
| Kidney NAT 4 | 8.2 | 14.9 |

CNS_neurodegeneration_v1.0 Summary: Ag4983/Ag6413/Ag6425/Ag6428/Ag6431/Ag6435/Ag6439/Ag6442/Ag6447 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

General_screening_panel v1.4 Summary: Ag4983 Highest expression of this gene is detected in a brain cancer SNB-19 cell line (CT=28). Moderate to low levels of expression of this gene is also seen in a number of cancer cell lines derived from gastric, colon, lung, renal breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

General_screening_panel_v1.5 Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening_panel_v1.6

Summary: Ag6413/Ag6425/Ag6428/Ag6431/Ag6435/Ag6439/Ag6964 Eight experiments with seven different probe and primer sets are in very good agreement. Highest expression of this gene is detected in a ovarian cancer IGROV-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6442 Expression of this gene is low/undetectable (CTs>34.9) across all of the samples on this panel (data not shown).

Panel 4.1D

Summary: Ag4983/Ag6425/Ag6428/Ag6431/Ag6435/Ag6439/Ag6447 Seven experiments with different probe and primer sets are in excellent agreement. Highest expression of this gene is detected in both resting and cytokine activated astrocytes (CTs=22-34.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

general oncology screening panel_v_{—}2.4 Summary: Ag4983/Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

AZ. CG56054-19: Integrin Alpha 7-Like Protein.

Expression of gene CG56054-19 was assessed using the primer-probe sets Ag6442, Ag6424, Ag6425, Ag6428, Ag6430, Ag6431, Ag6439, Ag6440, Ag6391 and Ag6964, described in Tables AZA, AZB, AZC, AZD, AZE, AZF, AZG, AZH, AZI and AZJ. Results of the RTQ-PCR runs are shown in Tables AZK, AZL, AZM, AZN and AZO.

TABLE AZA

Probe Name Ag6442

| | | | | |
|---|---|---|---|---|
| Primers | Sequences | Length | Start Position | SEQ ID No |

| | | | | |
|---|---|---|---|---|
| Forward | 5′-gatgtggacagtagggatagga-3 | 22 | 1951 | 738 |
| Probe | TET-5′-ccacctgagcagcaggagcct-3′-TAMRA | 21 | 1990 | 739 |
| Reverse | 5′-gcgcagtccagggtg-3′ | 15 | 2076 | 740 |

TABLE AZB

Probe Name Ag6424

| | | | | |
|---|---|---|---|---|
| Primers | Sequences | Length | Start Position | SEQ ID No |

| | | | | |
|---|---|---|---|---|
| Forward | 5′-ttgggttctgccagca-3′ | 16 | 641 | 741 |
| Probe | TET-5′-cacagctgccgccttctccc-3′-TAMRA | 20 | 660 | 742 |
| Reverse | 5′-aaaagcaaccccttccaa-3′ | 18 | 723 | 743 |

TABLE AZC

Probe Name Ag6425

| | | | | |
|---|---|---|---|---|
| Primers | Sequences | Length | Start Position | SEQ ID No |

| | | | | |
|---|---|---|---|---|
| Forward | 5′-cqgatgcacaccccat-3′ | 16 | 2573 | 744 |
| Probe | TET-5′-catcccgaqctgggcccc-3′-TAMRA | 18 | 2605 | 745 |
| Reverse | 5′-gccctggatgcccat-3′ | 15 | 2624 | 746 |

TABLE AZD

Probe Name Ag6428

| | | | | |
|---|---|---|---|---|
| Primers | Sequences | Length | Start Position | SEQ ID No |

| | | | | |
|---|---|---|---|---|
| Forward | 5′-cttcatctaccatgggagca-3′ | 20 | 1293 | 747 |
| Probe | TET-5′-ccttcacaggtgctggagggc-3′-TAMRA | 21 | 1333 | 748 |
| Reverse | 5′-agggagtagccgaagctct-3′ | 19 | 1370 | 749 |

TABLE AZE

Probe Name Ag6430

| | | | Start | |
|---|---|---|---|---|
| Primers | Sequences | Length | Position | SEQ ID No |
| Forward | 5′-gtgaccaacattgatagctcaga-3′ | 23 | 742 | 750 |
| Probe | TET-5′-ccccgaccagctggtgtataaaactttg-3′-TAMRA | 28 | 765 | 751 |
| Reverse | 5′-gggagccggtcagca-3′ | 15 | 798 | 752 |

TABLE AZF

Probe Name Ag6431

| | | | Start | |
|---|---|---|---|---|
| Primers | Sequences | Length | Position | SEQ ID No |
| Forward | 5′-aaacatcaacctggactgc-3′ | 19 | 2070 | 753 |
| Probe | TET-5′-tggtgttcagctgcccactctacag-3′-TAMRA | 25 | 2111 | 754 |
| Reverse | 5′-ccgcgcggtcaaa-3′ | 13 | 2137 | 755 |

TABLE AZG

Probe Name Ag6439

| | | | Start | |
|---|---|---|---|---|
| Primers | Sequences | Length | Position | SEQ ID No |
| Forward | 5′-ctgtggtggcagaaggagt-3′ | 19 | 2327 | 756 |
| Probe | TET-5′-ccctggtgggtcatcctcctg-3′-TAMRA | 21 | 2347 | 757 |
| Reverse | 5′-gaagaatcccatcttccacag-3′ | 21 | 2413 | 758 |

TABLE AZH

Probe Name Ag6440

| | | | Start | |
|---|---|---|---|---|
| Primers | Sequences | Length | Position | SEQ ID No |
| Forward | 5′-accatcctgaggaacaactg-3′ | 20 | 2530 | 759 |
| Probe | TET-5′-ctgacgggcatcccgagct-3′-TAMRA | 19 | 2597 | 760 |
| Reverse | 5′-ccctggatgcccatc-3′ | 15 | 2623 | 761 |

TABLE AZI

Probe Name Ag6391

| | | | | |
|---|---|---|---|---|
| Primers | Sequences | Length | Start Position | SEQ ID No |

| | | | | |
|---|---|---|---|---|
| Forward | 5′-tgcctccagggcctg-3′ | 15 | 1892 | 762 |
| Probe | TET-5′-ctcccaggcccaacatcctcca-3′-TAMRA | 22 | 1925 | 763 |
| Reverse | 5′-cgcctcctatccctactgtc- | 20 | 1957 | 764 |

TABLE AZJ

Probe Name Ag6964

| | | | | |
|---|---|---|---|---|
| Primers | Sequences | Length | Start Position | SEQ ID No |

| | | | | |
|---|---|---|---|---|
| Forward | 5′-ggccccagacatgca-3′ | 15 | 2156 | 765 |
| Probe | TET-5′-actctacagctttgaccgcgcgg-3′-TAMRA | 23 | 2127 | 766 |
| Reverse | 5′gccaactgtgtggtgttca-3′ | | 2101 | 767 |

TABLE AZK
CNS_neurodegeneration_v1.0

| | Rel. Exp. | Rel. Exp. | Rel. Exp. | Rel. Exp. | Rel. Exp. | Rel. Exp. | Rel. Exp. |
|---|---|---|---|---|---|---|---|
| | (%) Ag6425, | (%) Ag6428, | (%) Ag6430, | (%) Ag6431, | (%) Ag6439, | (%) Ag6440, | (%) Ag6442, |
| | Run | Run | Run | Run | Run | Run | Run |
| Tissue Name | 266937076 | 266937081 | 266937085 | 268030722 | 269254002 | 269254003 | 264979298 |
| AD 1 Hippo | 24.1 | 18.0 | 20.0 | 18.8 | 21.6 | 18.9 | 19.2 |
| AD 2 Hippo | 48.0 | 32.3 | 48.0 | 28.7 | 28.9 | 61.1 | 49.7 |
| AD 3 Hippo | 6.5 | 3.7 | 11.6 | 7.5 | 6.1 | 9.7 | 20.4 |
| AD 4 Hippo | 13.8 | 10.7 | 17.1 | 18.8 | 17.6 | 23.3 | 5.6 |
| AD 5 Hippo | 52.9 | 53.2 | 39.2 | 38.4 | 42.6 | 34.6 | 57.4 |
| AD 6 Hippo | 100.0 | 100.0 | 100.0 | 100.0 | 100.0 | 100.0 | 90.1 |
| Control 2 | 10.6 | 18.7 | 17.9 | 29.5 | 32.5 | 29.9 | 28.5 |
| Hippo | | | | | | | |
| Control 4 | 51.8 | 27.0 | 38.4 | 32.3 | 37.9 | 54.7 | 86.5 |
| Hippo | | | | | | | |
| Control | 9.8 | 4.6 | 10.2 | 6.0 | 6.4 | 5.8 | 0.0 |
| (Path) 3 | | | | | | | |
| Hippo | | | | | | | |
| AD 1 | 10.1 | 12.9 | 12.1 | 17.1 | 24.5 | 12.6 | 16.8 |
| Temporal | | | | | | | |
| Ctx | | | | | | | |
| AD 2 | 33.7 | 31.0 | 36.6 | 39.8 | 27.5 | 59.0 | 21.6 |
| Temporal | | | | | | | |
| Ctx | | | | | | | |
| AD 3 | 0.0 | 6.0 | 11.7 | 11.3 | 9.0 | 17.1 | 5.7 |
| Temporal | | | | | | | |
| Ctx | | | | | | | |
| AD 4 | 12.8 | 20.2 | 15.6 | 25.3 | 30.4 | 29.9 | 8.7 |
| Temporal | | | | | | | |
| Ctx | | | | | | | |
| AD 5 Inf | 59.0 | 39.2 | 43.8 | 36.3 | 41.8 | 41.8 | 73.7 |
| Temporal | | | | | | | |
| Ctx | | | | | | | |
| AD 5 Sup | 21.9 | 42.0 | 56.6 | 32.3 | 38.7 | 39.2 | 55.9 |
| Temporal | | | | | | | |
| Ctx | | | | | | | |
| AD 6 Inf | 73.7 | 49.3 | 40.9 | 46.7 | 47.6 | 48.6 | 76.8 |
| Temporal | | | | | | | |
| Ctx | | | | | | | |
| AD 6 Sup | 50.3 | 48.3 | 44.1 | 50.3 | 50.3 | 17.0 | 59.9 |
| Temporal | | | | | | | |
| Ctx | | | | | | | |
| Control 1 | 11.9 | 12.9 | 11.9 | 15.6 | 24.0 | 23.3 | 46.7 |
| Temporal | | | | | | | |
| Ctx | | | | | | | |
| Control 2 | 18.6 | 18.2 | 16.7 | 17.4 | 14.9 | 43.5 | 50.0 |
| Temporal | | | | | | | |
| Ctx | | | | | | | |
| Control 3 | 6.0 | 9.6 | 13.0 | 14.5 | 16.5 | 9.2 | 9.5 |
| Temporal | | | | | | | |
| Ctx | | | | | | | |
| Control 3 | 25.7 | 15.2 | 18.9 | 13.1 | 23.8 | 30.1 | 13.6 |
| Temporal | | | | | | | |
| Ctx | | | | | | | |
| Control | 18.0 | 27.0 | 32.5 | 30.6 | 39.8 | 51.1 | 46.0 |
| (Path) 1 | | | | | | | |
| Temporal | | | | | | | |
| Ctx | | | | | | | |
| Control | 18.4 | 16.0 | 19.5 | 20.4 | 24.8 | 7.2 | 0.0 |
| (Path) 2 | | | | | | | |
| Temporal | | | | | | | |
| Ctx | | | | | | | |
| Control | 5.6 | 7.5 | 12.9 | 10.9 | 11.9 | 9.9 | 31.0 |
| (Path) 3 | | | | | | | |
| Temporal | | | | | | | |
| Ctx | | | | | | | |
| Control | 16.8 | 17.1 | 19.8 | 18.2 | 21.6 | 14.9 | 39.5 |
| (Path) 4 | | | | | | | |
| Temporal | | | | | | | |
| Ctx | | | | | | | |
| AD 1 | 11.9 | 10.2 | 16.2 | 11.5 | 16.0 | 5.8 | 6.3 |
| Occipital Ctx | | | | | | | |
| AD 2 | 0.0 | 0.0 | 0.0 | 0.0 | 0.0 | 0.0 | 0.0 |
| Occipital Ctx | | | | | | | |
| (Missing) | | | | | | | |
| AD 3 | 8.3 | 6.4 | 11.7 | 8.8 | 10.2 | 7.8 | 4.9 |
| Occipital Ctx | | | | | | | |
| AD 4 | 5.8 | 13.0 | 12.6 | 17.9 | 18.6 | 35.4 | 11.1 |
| Occipital Ctx | | | | | | | |
| AD 5 | 25.2 | 25.3 | 16.7 | 22.5 | 22.7 | 16.6 | 42.3 |
| Occipital Ctx | | | | | | | |
| AD 6 | 19.8 | 20.2 | 17.8 | 17.0 | 22.1 | 23.5 | 14.8 |
| Occipital Ctx | | | | | | | |
| Control 1 | 6.6 | 6.0 | 11.3 | 8.7 | 7.2 | 15.2 | 8.8 |
| Occipital Ctx | | | | | | | |
| Control 2 | 15.7 | 26.4 | 24.8 | 33.2 | 29.3 | 35.8 | 82.4 |
| Occipital Ctx | | | | | | | |
| Control 3 | 5.7 | 10.7 | 16.4 | 17.1 | 19.2 | 4.4 | 8.8 |
| Occipital Ctx | | | | | | | |
| Control 4 | 21.6 | 12.0 | 12.1 | 12.6 | 13.6 | 12.9 | 24.0 |
| Occipital Ctx | | | | | | | |
| Control | 28.3 | 35.6 | 32.8 | 36.1 | 39.5 | 22.4 | 100.0 |
| (Path) 1 | | | | | | | |
| Occipital Ctx | | | | | | | |
| Control | 49.7 | 6.7 | 9.6 | 7.9 | 7.0 | 5.0 | 9.3 |
| (Path) 2 | | | | | | | |
| Occipital Ctx | | | | | | | |
| Control | 0.0 | 5.4 | 8.4 | 6.0 | 5.9 | 6.7 | 4.1 |
| (Path) 3 | | | | | | | |
| Occipital Ctx | | | | | | | |
| Control | 6.6 | 13.2 | 15.9 | 10.2 | 11.4 | 11.9 | 32.8 |
| (Path) 4 | | | | | | | |
| Occipital Ctx | | | | | | | |
| Control 1 | 8.8 | 8.8 | 15.2 | 16.3 | 15.7 | 33.2 | 9.2 |
| Parietal Ctx | | | | | | | |
| Control 2 | 14.5 | 34.4 | 39.5 | 28.3 | 37.1 | 17.4 | 28.1 |
| Parietal Ctx | | | | | | | |
| Control 3 | 19.9 | 11.5 | 14.5 | 8.7 | 10.8 | 21.6 | 9.1 |
| Parietal Ctx | | | | | | | |
| Control | 37.6 | 34.2 | 33.4 | 39.2 | 37.9 | 47.3 | 69.3 |
| (Path) 1 | | | | | | | |
| Parietal Ctx | | | | | | | |
| Control | 16.6 | 19.6 | 20.0 | 22.5 | 18.7 | 17.1 | 37.6 |
| (Path) 2 | | | | | | | |
| Parietal Ctx | | | | | | | |
| Control | 0.0 | 3.9 | 15.0 | 7.1 | 12.0 | 11.7 | 10.4 |
| (Path) 3 | | | | | | | |
| Parietal Ctx | | | | | | | |
| Control | 18.2 | 24.8 | 28.3 | 8.8 | 27.9 | 29.3 | 27.5 |
| (Path) 4 | | | | | | | |
| Parietal Ctx | | | | | | | |

TABLE AZL
General_screening_panel_v1.5

| | Rel. Exp. (%) |
|---|---|
| | Ag6442, Run |
| Tissue Name | 264979530 |
| Adipose | 3.2 |
| Melanoma* Hs688(A).T | 0.5 |
| Melanoma* Hs688(B).T | 0.5 |
| Melanoma* M14 | 0.7 |
| Melanoma* LOXIMVI | 0.0 |
| Melanoma* SK-MEL-5 | 8.9 |
| Squamous cell carcinoma SCC-4 | 0.0 |
| Testis Pool | 3.5 |
| Prostate ca.* (bone met) PC-3 | 0.1 |
| Prostate Pool | 3.1 |
| Placenta | 0.4 |
| Uterus Pool | 5.4 |
| Ovarian ca. OVCAR-3 | 0.4 |
| Ovarian ca. SK-OV-3 | 0.1 |
| Ovarian ca. OVCAR-4 | 0.3 |
| Ovarian ca. OVCAR-5 | 0.8 |
| Ovarian ca. IGROV-1 | 66.0 |
| Ovarian ca. OVCAR-8 | 11.2 |
| Ovary | 2.0 |
| Breast ca. MCF-7 | 0.1 |
| Breast ca. MDA-MB-231 | 0.2 |
| Breast ca. BT 549 | 0.4 |
| Breast ca. T47D | 0.0 |
| Breast ca. MDA-N | 0.5 |
| Breast Pool | 7.4 |
| Trachea | 2.4 |
| Lung | 3.5 |
| Fetal Lung | 3.8 |
| Lung ca. NCI-N417 | 1.6 |
| Lung ca. LX-1 | 1.4 |
| Lung ca. NCI-H146 | 0.4 |
| Lung ca. SHP-77 | 2.0 |
| Lung ca. A549 | 0.2 |
| Lung ca. NCI-H526 | 0.6 |
| Lung ca. NCI-H23 | 2.0 |
| Lung ca. NCI-H460 | 0.1 |
| Lung ca. HOP-62 | 0.6 |
| Lung ca. NCI-H522 | 1.1 |
| Liver | 0.2 |
| Fetal Liver | 0.2 |
| Liver ca. HepG2 | 0.0 |
| Kidney Pool | 15.6 |
| Fetal Kidney | 1.0 |
| Renal ca. 786-0 | 0.2 |
| Renal ca. A498 | 0.2 |
| Renal ca. ACHN | 0.2 |
| Renal ca. UO-31 | 0.4 |
| Renal ca. TK-10 | 0.8 |
| Bladder | 2.1 |
| Gastric ca. (liver met.) NCI-N87 | 0.7 |
| Gastric ca. KATO III | 0.2 |
| Colon ca. SW-948 | 0.1 |
| Colon ca. SW480 | 17.7 |
| Colon ca.* (SW480 met) SW620 | 7.9 |
| Colon ca. HT29 | 0.5 |
| Colon ca. HCT-116 | 2.4 |
| Colon ca. CaCo-2 | 10.2 |
| Colon cancer tissue | 10.7 |
| Colon ca. SW1116 | 1.3 |
| Colon ca. Colo-205 | 0.0 |
| Colon ca. SW-48 | 0.7 |
| Colon Pool | 6.3 |
| Small Intestine Pool | 5.2 |
| Stomach Pool | 4.3 |
| Bone Marrow Pool | 3.3 |
| Fetal Heart | 7.6 |
| Heart Pool | 13.3 |
| Lymph Node Pool | 7.1 |
| Fetal Skeletal Muscle | 16.5 |
| Skeletal Muscle Pool | 100.0 |
| Spleen Pool | 1.9 |
| Thymus Pool | 5.5 |
| CNS cancer (glio/astro) U87-MG | 7.4 |
| CNS cancer (glio/astro) U-118-MG | 2.6 |
| CNS cancer (neuro; met) SK-N-AS | 1.2 |
| CNS cancer (astro) SF-539 | 0.2 |
| CNS cancer (astro) SNB-75 | 6.7 |
| CNS cancer (glio) SNB-19 | 63.7 |
| CNS cancer (glio) SF-295 | 4.0 |
| Brain (Amygdala) Pool | 5.0 |
| Brain (cerebellum) | 3.3 |
| Brain (fetal) | 1.9 |
| Brain (Hippocampus) Pool | 5.7 |
| Cerebral Cortex Pool | 4.6 |
| Brain (Substantia nigra) Pool | 5.1 |
| Brain (Thalamus) Pool | 3.7 |
| Brain (whole) | 3.2 |
| Spinal Cord Pool | 9.0 |
| Adrenal Gland | 3.1 |
| Pituitary gland Pool | 0.7 |
| Salivary Gland | 0.7 |
| Thyroid (female) | 1.0 |
| Pancreatic ca. CAPAN2 | 0.5 |
| Pancreas Pool | 8.8 |

TABLE AZM
General_screening_panel_v1.6

| | Rel. Exp. | Rel. Exp. | Rel. Exp. | Rel. Exp. | Rel. Exp. | Rel. Exp. | Rel. Exp. | Rel. Exp. | Rel. Exp. |
|---|---|---|---|---|---|---|---|---|---|
| | (%) Ag6424, | (%) Ag6425, | (%) Ag6428, | (%) Ag6430, | (%) Ag6431, | (%) Ag6431, | (%) Ag6439, | (%) Ag6440, | (%) Ag6964, |
| | Run | Run | Run | Run | Run | Run | Run | Run | Run |
| Tissue Name | 277221719 | 277221721 | 277222439 | 277222443 | 77633568 | 278389390 | 277223175 | 2772231 | 278388946 |
| Adipose | 0.0 | 2.6 | 20.0 | 8.2 | 17.4 | 13.8 | 17.3 | 3.7 | 18.8 |
| Melanoma* | 0.0 | 0.0 | 2.0 | 0.5 | 0.8 | 0.9 | 0.4 | 0.0 | 0.7 |
| Hs688(A).T | | | | | | | | | |
| Melanoma* | 0.0 | 0.2 | 4.1 | 0.6 | 2.5 | 2.2 | 2.9 | 0.8 | 2.4 |
| Hs688(B).T | | | | | | | | | |
| Melanoma* | 0.0 | 0.0 | 0.7 | 0.7 | 0.4 | 0.4 | 0.4 | 0.0 | 0.7 |
| M14 | | | | | | | | | |
| Melanoma* | 0.0 | 0.0 | 0.1 | 0.0 | 0.0 | 0.0 | 0.0 | 0.0 | 0.1 |
| LOXIMVI | | | | | | | | | |
| Melanoma* | 0.0 | 2.2 | 30.4 | 22.5 | 18.2 | 14.6 | 18.3 | 3.0 | 15.9 |
| SK-MEL-5 | | | | | | | | | |
| Squamous | 0.0 | 0.0 | 0.1 | 0.3 | 0.1 | 0.2 | 0.0 | 0.0 | 0.1 |
| cell | | | | | | | | | |
| carcinoma | | | | | | | | | |
| SCC-4 | | | | | | | | | |
| Testis Pool | 0.0 | 3.5 | 8.8 | 4.2 | 10.4 | 9.0 | 9.1 | 3.0 | 9.9 |
| Prostate ca.* | 0.0 | 0.5 | 2.5 | 1.0 | 1.9 | 1.8 | 1.3 | 1.2 | 4.3 |
| (bone met) | | | | | | | | | |
| PC-3 | | | | | | | | | |
| Prostate Pool | 0.0 | 1.0 | 11.5 | 8.5 | 11.3 | 12.1 | 28.5 | 2.1 | 10.0 |
| Placenta | 0.0 | 0.0 | 0.7 | 0.1 | 0.1 | 0.1 | 0.5 | 0.0 | 0.4 |
| Uterus Pool | 0.0 | 1.5 | 4.5 | 2.6 | 4.6 | 4.5 | 5.3 | 2.3 | 4.1 |
| Ovarian ca. | 0.0 | 0.3 | 1.1 | 0.8 | 0.7 | 1.1 | 1.6 | 0.4 | 4.0 |
| OVCAR-3 | | | | | | | | | |
| Ovarian ca. | 0.0 | 0.2 | 1.7 | 1.5 | 0.8 | 0.9 | 1.3 | 0.5 | 1.7 |
| SK-OV-3 | | | | | | | | | |
| Ovarian ca. | 0.0 | 0.0 | 0.9 | 0.5 | 0.4 | 0.8 | 0.9 | 0.0 | 0.5 |
| OVCAR-4 | | | | | | | | | |
| Ovarian ca. | 0.0 | 1.3 | 2.9 | 1.5 | 1.3 | 1.7 | 1.4 | 4.2 | 7.9 |
| OVCAR-5 | | | | | | | | | |
| Ovarian ca. | 100.0 | 100.0 | 77.9 | 90.8 | 84.7 | 97.9 | 69.3 | 100.0 | 75.8 |
| IGROV-1 | | | | | | | | | |
| Ovarian ca. | 5.6 | 21.9 | 14.0 | 11.9 | 15.6 | 14.6 | 17.3 | 18.2 | 16.7 |
| OVCAR-8 | | | | | | | | | |
| Ovary | 0.0 | 0.3 | 5.2 | 2.1 | 3.1 | 2.3 | 2.8 | 0.8 | 2.4 |
| Breast ca. | 0.0 | 0.0 | 0.3 | 0.4 | 0.1 | 0.2 | 0.5 | 0.3 | 0.5 |
| MCF-7 | | | | | | | | | |
| Breast ca. | 0.0 | 0.0 | 0.4 | 0.4 | 0.2 | 0.2 | 0.2 | 0.0 | 0.3 |
| MDA-MB- | | | | | | | | | |
| 231 | | | | | | | | | |
| Breast ca. | 0.0 | 0.0 | 0.5 | 0.3 | 0.1 | 0.5 | 0.6 | 0.0 | 0.4 |
| BT 549 | | | | | | | | | |
| Breast ca. | 0.0 | 0.0 | 0.5 | 0.3 | 0.2 | 0.3 | 0.4 | 0.3 | 0.5 |
| T47D | | | | | | | | | |
| Breast ca. | 0.0 | 0.0 | 0.7 | 0.7 | 0.6 | 0.6 | 0.6 | 0.3 | 0.8 |
| MDA-N | | | | | | | | | |
| Breast Pool | 0.0 | 4.1 | 21.8 | 19.5 | 14.6 | 10.7 | 12.2 | 3.5 | 16.7 |
| Trachea | 0.0 | 0.7 | 8.4 | 2.9 | 4.8 | 4.2 | 4.7 | 1.4 | 5.6 |
| Lung | 0.0 | 0.7 | 2.3 | 1.3 | 4.2 | 3.2 | 3.9 | 5.3 | 5.1 |
| Fetal Lung | 0.0 | 0.3 | 9.1 | 4.0 | 5.0 | 4.8 | 5.3 | 2.9 | 6.1 |
| Lung ca. | 2.0 | 0.9 | 3.5 | 2.7 | 3.3 | 2.6 | 4.0 | 2.0 | 2.3 |
| NCI-N417 | | | | | | | | | |
| Lung ca. | 3.1 | 2.7 | 6.5 | 7.0 | 5.0 | 3.5 | 4.9 | 6.3 | 44.1 |
| LX-1 | | | | | | | | | |
| Lung ca. | 0.0 | 0.0 | 0.3 | 0.5 | 0.1 | 0.2 | 0.1 | 0.0 | 0.1 |
| NCI-H146 | | | | | | | | | |
| Lung ca. | 2.3 | 0.4 | 6.8 | 6.3 | 5.3 | 4.5 | 4.5 | 0.8 | 3.8 |
| SHP-77 | | | | | | | | | |
| Lung ca. | 0.0 | 2.6 | 0.9 | 0.3 | 0.0 | 0.4 | 0.6 | 2.2 | 4.7 |
| A549 | | | | | | | | | |
| Lung ca. | 0.0 | 0.0 | 0.9 | 0.7 | 0.6 | 0.3 | 0.4 | 0.3 | 0.5 |
| NCI-H526 | | | | | | | | | |
| Lung ca. | 0.0 | 1.0 | 4.6 | 4.5 | 4.8 | 3.2 | 2.9 | 2.3 | 10.3 |
| NCI-H23 | | | | | | | | | |
| Lung ca. | 0.0 | 0.0 | 0.2 | 0.2 | 0.1 | 0.3 | 0.0 | 0.0 | 0.3 |
| NCI-H460 | | | | | | | | | |
| Lung ca. | 0.0 | 0.0 | 0.5 | 0.6 | 1.0 | 0.6 | 0.5 | 0.0 | 0.7 |
| HOP-62 | | | | | | | | | |
| Lung ca. | 0.0 | 0.6 | 2.3 | 2.4 | 1.7 | 1.3 | 3.3 | 2.5 | 8.9 |
| NCI-H522 | | | | | | | | | |
| Liver | 0.0 | 0.0 | 0.0 | 0.1 | 0.0 | 0.0 | 0.1 | 0.4 | 2.0 |
| Fetal Liver | 0.0 | 0.3 | 0.1 | 0.6 | 0.6 | 0.5 | 0.8 | 0.8 | 8.2 |
| Liver ca. | 0.0 | 0.3 | 0.2 | 0.1 | 0.0 | 0.2 | 0.1 | 0.9 | 2.4 |
| HepG2 | | | | | | | | | |
| Kidney Pool | 6.5 | 0.0 | 47.0 | 34.9 | 33.9 | 28.1 | 43.2 | 14.6 | 32.8 |
| Fetal Kidney | 0.0 | 0.0 | 4.9 | 5.1 | 4.1 | 4.0 | 5.8 | 3.4 | 11.5 |
| Renal ca. | 0.0 | 0.0 | 0.2 | 0.2 | 0.3 | 0.1 | 0.3 | 0.0 | 0.9 |
| 786-0 | | | | | | | | | |
| Renal ca. | 0.0 | 1.8 | 0.2 | 0.1 | 0.0 | 0.3 | 0.5 | 3.8 | 8.5 |
| A498 | | | | | | | | | |
| Renal ca. | 0.0 | 0.5 | 2.5 | 0.7 | 1.7 | 1.5 | 1.2 | 0.5 | 2.5 |
| ACHN | | | | | | | | | |
| Renal ca. | 0.0 | 0.0 | 0.5 | 0.3 | 0.2 | 0.2 | 0.6 | 0.0 | 0.3 |
| UO-31 | | | | | | | | | |
| Renal ca. | 0.0 | 0.4 | 3.1 | 2.5 | 2.0 | 1.9 | 2.1 | 0.5 | 4.6 |
| TK-10 | | | | | | | | | |
| Bladder | 0.0 | 0.0 | 5.9 | 3.0 | 5.5 | 5.1 | 8.3 | 0.9 | 6.7 |
| Gastric ca. | 0.0 | 0.0 | 1.7 | 1.7 | 0.9 | 1.2 | 1.1 | 0.8 | 6.7 |
| (liver met.) | | | | | | | | | |
| NCI-N87 | | | | | | | | | |
| Gastric ca. | 0.0 | 0.5 | 0.8 | 0.4 | 0.2 | 0.3 | 0.4 | 0.4 | 0.9 |
| KATO III | | | | | | | | | |
| Colon ca. | 0.0 | 1.5 | 0.2 | 0.0 | 0.2 | 0.2 | 0.3 | 2.2 | 1.2 |
| SW-948 | | | | | | | | | |
| Colon ca. | 9.5 | 5.2 | 41.8 | 39.0 | 27.0 | 23.3 | 23.0 | 6.3 | 33.7 |
| SW480 | | | | | | | | | |
| Colon ca.* | 7.7 | 4.8 | 16.4 | 15.5 | 12.8 | 10.3 | 6.1 | 7.2 | 25.0 |
| (SW480 | | | | | | | | | |
| met) SW620 | | | | | | | | | |
| Colon ca. | 0.0 | 0.0 | 0.0 | 0.0 | 0.2 | 0.2 | 0.0 | 0.3 | 0.3 |
| HT29 | | | | | | | | | |
| Colon ca. | 1.6 | 0.2 | 3.2 | 3.8 | 2.5 | 2.0 | 2.1 | 0.6 | 4.3 |
| HCT-116 | | | | | | | | | |
| Colon ca. | 10.4 | 3.6 | 27.0 | 22.2 | 19.1 | 16.7 | 18.3 | 6.5 | 38.2 |
| CaCo-2 | | | | | | | | | |
| Colon cancer | 0.0 | 3.3 | 11.0 | 6.5 | 11.9 | 7.6 | 7.7 | 4.4 | 20.4 |
| tissue | | | | | | | | | |
| Colon ca. | 0.0 | 3.0 | 2.5 | 1.7 | 2.0 | 1.5 | 1.8 | 2.1 | 6.0 |
| SW1116 | | | | | | | | | |
| Colon ca. | 0.0 | 0.4 | 0.3 | 0.2 | 0.2 | 0.0 | 0.2 | 1.3 | 0.8 |
| Colo-205 | | | | | | | | | |
| Colon ca. | 0.0 | 3.6 | 1.4 | 1.3 | 1.5 | 1.5 | 1.4 | 3.0 | 2.6 |
| SW-48 | | | | | | | | | |
| Colon Pool | 0.0 | 5.0 | 28.1 | 28.7 | 23.2 | 18.7 | 25.5 | 8.1 | 20.6 |
| Small | 0.0 | 1.7 | 17.1 | 10.5 | 11.2 | 13.0 | 12.8 | 2.0 | 10.4 |
| Intestine | | | | | | | | | |
| Pool | | | | | | | | | |
| Stomach | 0.0 | 2.3 | 14.3 | 6.2 | 9.5 | 9.3 | 8.5 | 4.2 | 10.7 |
| Pool | | | | | | | | | |
| Bone | 0.0 | 1.6 | 14.3 | 11.3 | 10.2 | 8.7 | 18.7 | 3.5 | 12.5 |
| Marrow Pool | | | | | | | | | |
| Fetal Heart | 0.0 | 2.3 | 25.5 | 24.3 | 24.5 | 21.8 | 33.7 | 8.6 | 20.7 |
| Heart Pool | 5.2 | 7.0 | 29.7 | 23.0 | 25.9 | 17.2 | 33.7 | 10.7 | 26.1 |
| Lymph Node | 0.0 | 6.1 | 33.7 | 30.4 | 22.1 | 23.7 | 19.9 | 6.7 | 24.7 |
| Pool | | | | | | | | | |
| Fetal | 36.9 | 5.2 | 54.3 | 46.7 | 48.6 | 46.3 | 19.1 | 19.2 | 50.7 |
| Skeletal | | | | | | | | | |
| Muscle | | | | | | | | | |
| Skeletal | 12.3 | 9.2 | 29.3 | 21.5 | 29.5 | 25.9 | 22.1 | 22.7 | 32.3 |
| Muscle Pool | | | | | | | | | |
| Spleen Pool | 0.0 | 0.0 | 1.9 | 2.0 | 2.0 | 1.7 | 2.7 | 0.6 | 3.1 |
| Thymus | 0.0 | 2.0 | 10.4 | 7.5 | 8.1 | 9.4 | 7.7 | 3.1 | 7.0 |
| Pool | | | | | | | | | |
| CNS cancer | 1.6 | 1.5 | 14.9 | 6.1 | 10.7 | 10.0 | 10.9 | 2.2 | 14.1 |
| (glio/astro) | | | | | | | | | |
| U87-MG | | | | | | | | | |
| CNS cancer | 0.0 | 0.3 | 4.7 | 2.9 | 3.8 | 3.1 | 3.8 | 0.8 | 5.8 |
| (glio/astro) | | | | | | | | | |
| U-118-MG | | | | | | | | | |
| CNS cancer | 0.0 | 0.0 | 2.6 | 1.7 | 2.1 | 1.0 | 1.4 | 0.5 | 2.6 |
| (neuro; met) | | | | | | | | | |
| SK-N-AS | | | | | | | | | |
| CNS cancer | 0.0 | 0.0 | 0.0 | 0.2 | 0.1 | 0.2 | 0.1 | 0.2 | 0.1 |
| (astro) SF- | | | | | | | | | |
| 539 | | | | | | | | | |
| CNS cancer | 1.9 | 1.1 | 14.9 | 5.9 | 6.5 | 10.0 | 11.7 | 2.8 | 9.7 |
| (astro) SNB- | | | | | | | | | |
| 75 | | | | | | | | | |
| CNS cancer | 84.1 | 79.0 | 100.0 | 100.0 | 100.0 | 100.0 | 100.0 | 97.9 | 100.0 |
| (glio) SNB- | | | | | | | | | |
| 19 | | | | | | | | | |
| CNS cancer | 1.8 | 0.0 | 11.3 | 9.0 | 8.0 | 7.8 | 8.2 | 1.5 | 14.8 |
| (glio) SF- | | | | | | | | | |
| 295 | | | | | | | | | |
| Brain | 2.3 | 0.8 | 7.7 | 6.9 | 6.2 | 4.8 | 8.0 | 4.4 | 5.3 |
| (Amygdala) | | | | | | | | | |
| Pool | | | | | | | | | |
| Brain | 6.6 | 0.4 | 19.8 | 11.1 | 10.7 | 9.7 | 8.8 | 1.2 | 9.7 |
| (cerebellum) | | | | | | | | | |
| Brain (fetal) | 3.0 | 0.7 | 12.7 | 11.5 | 6.6 | 5.6 | 6.8 | 2.1 | 6.4 |
| Brain | 3.1 | 3.2 | 11.7 | 11.0 | 8.6 | 6.9 | 11.0 | 4.3 | 10.2 |
| (Hippocampus) | | | | | | | | | |
| Pool | | | | | | | | | |
| Cerebral | 1.7 | 0.6 | 11.0 | 7.5 | 7.5 | 0.7 | 11.6 | 2.0 | 8.7 |
| Cortex Pool | | | | | | | | | |
| Brain | 1.8 | 2.2 | 11.7 | 8.5 | 10.4 | 4.7 | 10.0 | 2.0 | 9.3 |
| (Substantia | | | | | | | | | |
| nigra) Pool | | | | | | | | | |
| Brain | 0.0 | 2.7 | 13.2 | 10.0 | 9.3 | 0.2 | 9.7 | 2.8 | 8.7 |
| (Thalamus) | | | | | | | | | |
| Pool | | | | | | | | | |
| Brain | 0.0 | 0.4 | 10.6 | 8.0 | 5.8 | 0.3 | 5.6 | 1.9 | 8.7 |
| (whole) | | | | | | | | | |
| Spinal Cord | 3.2 | 2.3 | 14.7 | 12.8 | 11.0 | 7.6 | 12.2 | 4.2 | 9.0 |
| Pool | | | | | | | | | |
| Adrenal | 0.0 | 0.3 | 9.9 | 6.1 | 3.9 | 3.7 | 4.8 | 0.9 | 4.1 |
| Gland | | | | | | | | | |
| Pituitary | 0.0 | 0.0 | 1.1 | 0.8 | 1.2 | 1.1 | 1.4 | 0.6 | 0.5 |
| gland Pool | | | | | | | | | |
| Salivary | 0.0 | 0.0 | 1.8 | 1.1 | 1.3 | 0.9 | 1.1 | 0.0 | 1.0 |
| Gland | | | | | | | | | |
| Thyroid | 0.0 | 0.3 | 3.1 | 0.8 | 2.5 | 2.5 | 1.9 | 1.3 | 2.3 |
| (female) | | | | | | | | | |
| Pancreatic | 0.0 | 0.0 | 0.8 | 0.8 | 0.7 | 0.6 | 0.7 | 0.6 | 2.2 |
| ca. CAPAN2 | | | | | | | | | |
| Pancreas | 0.0 | 0.0 | 2.0 | 1.1 | 1.1 | 1.6 | 3.2 | 1.0 | 2.3 |
| Pool | | | | | | | | | |

TABLE AZN
Panel 4.1D

| | Rel. Exp. | Rel. Exp. | Rel. Exp. | Rel. Exp. | Rel. Exp. | Rel. Exp. |
|---|---|---|---|---|---|---|
| | (%) Ag6425, | (%) Ag6428, | (%) Ag6430, | (%) Ag6431, | (%) Ag6439, | (%) Ag6440, |
| | Run | Run | Run | Run | Run | Run |
| Tissue Name | 268713999 | 268767535 | 268767563 | 268767577 | 268760823 | 268760825 |
| Secondary Th1 act | 0.0 | 1.3 | 0.0 | 0.7 | 0.0 | 0.0 |
| Secondary Th2 act | 0.0 | 1.2 | 0.0 | 0.8 | 0.0 | 0.0 |
| Secondary Tr1 act | 0.0 | 0.0 | 0.0 | 0.7 | 0.0 | 0.0 |
| Secondary Th1 rest | 0.0 | 0.0 | 0.0 | 0.0 | 0.0 | 0.0 |
| Secondary Th2 rest | 0.0 | 0.0 | 0.0 | 0.0 | 0.0 | 0.0 |
| Secondary Tr1 rest | 0.0 | 0.4 | 0.0 | 0.0 | 0.0 | 0.0 |
| Primary Th1 act | 0.0 | 0.0 | 0.0 | 0.0 | 0.0 | 0.0 |
| Primary Th2 act | 0.0 | 0.3 | 0.0 | 0.4 | 0.0 | 0.0 |
| Primary Tr1 act | 0.0 | 0.7 | 0.0 | 0.7 | 0.0 | 0.0 |
| Primary Th1 rest | 0.0 | 0.1 | 0.0 | 0.3 | 1.2 | 0.0 |
| Primary Th2 rest | 0.0 | 0.4 | 0.0 | 0.2 | 0.0 | 0.0 |
| Primary Tr1 rest | 0.0 | 0.0 | 0.0 | 0.0 | 0.0 | 0.0 |
| CD45RA CD4 | 0.0 | 5.4 | 0.0 | 2.4 | 2.6 | 0.0 |
| lymphocyte act | | | | | | |
| CD45RO CD4 | 0.0 | 1.5 | 0.0 | 0.7 | 2.3 | 0.0 |
| lymphocyte act | | | | | | |
| CD8 lymphocyte act | 0.0 | 0.7 | 0.0 | 0.0 | 0.0 | 0.0 |
| Secondary CD8 | 0.0 | 8.8 | 0.0 | 0.0 | 0.0 | 0.0 |
| lymphocyte rest | | | | | | |
| Secondary CD8 | 0.0 | 0.4 | 0.0 | 0.3 | 0.0 | 0.0 |
| lymphocyte act | | | | | | |
| CD4 lymphocyte | 0.0 | 0.5 | 0.0 | 0.4 | 0.0 | 0.0 |
| none | | | | | | |
| 2ry | 0.0 | 0.0 | 0.0 | 0.0 | 1.2 | 0.0 |
| Th1/Th2/Tr1_anti- | | | | | | |
| CD95 CH11 | | | | | | |
| LAK cells rest | 2.7 | 11.8 | 0.1 | 3.8 | 15.2 | 0.0 |
| LAK cells IL-2 | 0.0 | 0.0 | 0.0 | 0.0 | 0.0 | 0.0 |
| LAK cells IL-2 + | 0.0 | 0.0 | 0.0 | 0.0 | 0.0 | 0.0 |
| IL-12 | | | | | | |
| LAK cells IL-2 + | 0.0 | 0.0 | 0.0 | 0.0 | 0.0 | 0.0 |
| IFN gamma | | | | | | |
| LAK cells IL-2 + | 0.0 | 0.0 | 0.0 | 0.0 | 0.0 | 0.0 |
| IL-18 | | | | | | |
| LAK cells | 15.7 | 15.1 | 0.1 | 6.3 | 9.0 | 52.9 |
| PMA/ionomycin | | | | | | |
| NK Cells IL-2 rest | 0.0 | 3.4 | 0.0 | 2.5 | 1.4 | 0.0 |
| Two Way MLR 3 | 0.0 | 2.2 | 0.0 | 1.3 | 1.4 | 0.0 |
| day | | | | | | |
| Two Way MLR 5 | 0.0 | 0.8 | 0.0 | 0.9 | 0.0 | 0.0 |
| day | | | | | | |
| Two Way MLR 7 | 13.2 | 1.1 | 0.0 | 2.6 | 3.7 | 0.0 |
| day | | | | | | |
| PBMC rest | 0.0 | 0.0 | 0.0 | 0.0 | 0.0 | 0.0 |
| PBMC PWM | 0.0 | 1.3 | 0.0 | 0.0 | 0.0 | 0.0 |
| PBMC PHA-L | 0.0 | 0.6 | 0.0 | 0.7 | 0.0 | 0.0 |
| Ramos (B cell) none | 0.0 | 0.0 | 0.0 | 0.0 | 0.0 | 0.0 |
| Ramos (B cell) | 0.0 | 0.7 | 0.0 | 0.2 | 0.0 | 0.0 |
| ionomycin | | | | | | |
| B lymphocytes | 0.0 | 0.0 | 0.0 | 0.0 | 0.0 | 0.0 |
| PWM | | | | | | |
| B lymphocytes | 0.0 | 0.9 | 0.0 | 0.0 | 0.0 | 0.0 |
| CD40L and IL-4 | | | | | | |
| EOL-1 dbcAMP | 9.1 | 29.1 | 0.1 | 8.1 | 68.8 | 0.0 |
| EOL-1 dbcAMP | 0.0 | 0.0 | 0.0 | 2.7 | 1.8 | 0.0 |
| PMA/ionomycin | | | | | | |
| Dendritic cells none | 13.8 | 4.1 | 0.0 | 5.3 | 0.0 | 0.0 |
| Dendritic cells LPS | 0.0 | 1.0 | 0.0 | 0.7 | 0.0 | 0.0 |
| Dendritic cells anti- | 3.3 | 0.5 | 0.0 | 0.2 | 0.0 | 0.0 |
| CD40 | | | | | | |
| Monocytes rest | 0.0 | 0.4 | 0.0 | 0.0 | 0.0 | 0.0 |
| Monocytes LPS | 0.0 | 5.7 | 0.0 | 1.8 | 2.6 | 0.0 |
| Macrophages rest | 0.0 | 0.6 | 0.0 | 0.6 | 0.0 | 0.0 |
| Macrophages LPS | 0.0 | 5.4 | 0.1 | 6.3 | 9.2 | 0.0 |
| HUVEC none | 0.0 | 0.0 | 0.0 | 0.0 | 0.0 | 0.0 |
| HUVEC starved | 0.0 | 0.0 | 0.0 | 0.3 | 0.0 | 0.0 |
| HUVEC IL-1beta | 0.0 | 0.0 | 0.0 | 0.5 | 0.0 | 0.0 |
| HUVEC IFN | 0.0 | 0.0 | 0.0 | 0.0 | 0.0 | 0.0 |
| gamma | | | | | | |
| HUVEC TNF alpha + | 0.0 | 0.0 | 0.0 | 0.0 | 0.0 | 0.0 |
| IFN gamma | | | | | | |
| HUVEC TNF alpha + | 0.0 | 0.0 | 0.0 | 0.4 | 0.0 | 0.0 |
| IL4 | | | | | | |
| HUVEC IL-11 | 0.0 | 0.4 | 0.0 | 0.3 | 0.0 | 0.0 |
| Lung Microvascular | 0.0 | 0.4 | 0.0 | 0.0 | 0.0 | 0.0 |
| EC none | | | | | | |
| Lung Microvascular | 0.0 | 0.0 | 0.0 | 0.0 | 0.0 | 0.0 |
| EC TNFalpha + | | | | | | |
| IL-1beta | | | | | | |
| Microvascular | 0.0 | 0.0 | 0.0 | 0.0 | 0.0 | 0.0 |
| Dermal EC none | | | | | | |
| Microsvasular | 0.0 | 0.0 | 0.0 | 0.0 | 0.0 | 0.0 |
| Dermal EC | | | | | | |
| TNFalpha + | | | | | | |
| IL-1beta | | | | | | |
| Bronchial | 0.0 | 0.0 | 0.0 | 0.0 | 0.0 | 0.0 |
| epithelium | | | | | | |
| TNFalpha + | | | | | | |
| IL1beta | | | | | | |
| Small airway | 0.0 | 0.0 | 0.0 | 0.0 | 0.0 | 0.0 |
| epithelium none | | | | | | |
| Small airway | 0.0 | 0.0 | 0.0 | 0.0 | 0.0 | 0.0 |
| epithelium | | | | | | |
| TNFalpha + | | | | | | |
| IL-1beta | | | | | | |
| Coronery artery | 0.0 | 0.0 | 0.0 | 0.0 | 0.0 | 0.0 |
| SMC rest | | | | | | |
| Coronery artery | 6.2 | 0.3 | 0.0 | 1.5 | 0.0 | 0.0 |
| SMC TNFalpha + | | | | | | |
| IL-1beta | | | | | | |
| Astrocytes rest | 100.0 | 100.0 | 12.0 | 100.0 | 100.0 | 100.0 |
| Astrocytes | 74.2 | 97.3 | 100.0 | 74.7 | 95.9 | 95.3 |
| TNFalpha + | | | | | | |
| IL-1beta | | | | | | |
| KU-812 (Basophil) | 0.0 | 0.0 | 0.0 | 0.4 | 0.0 | 0.0 |
| rest | | | | | | |
| KU-812 (Basophil) | 0.0 | 0.0 | 0.0 | 0.0 | 0.0 | 0.0 |
| PMA/ionomycin | | | | | | |
| CCD1106 | 0.0 | 0.0 | 0.0 | 0.8 | 0.0 | 0.0 |
| (Keratinocytes) none | | | | | | |
| CCD1106 | 0.0 | 0.0 | 0.0 | 0.0 | 0.0 | 0.0 |
| (Keratinocytes) | | | | | | |
| TNFalpha + | | | | | | |
| IL-1beta | | | | | | |
| Liver cirrhosis | 4.6 | 2.6 | 0.0 | 6.7 | 8.5 | 0.0 |
| NCI-H292 none | 0.0 | 1.7 | 0.0 | 0.6 | 0.0 | 0.0 |
| NCI-H292 IL-4 | 0.0 | 0.0 | 0.0 | 0.5 | 0.0 | 0.0 |
| NCI-H292 IL-9 | 0.0 | 0.7 | 0.0 | 0.5 | 0.0 | 0.0 |
| NCI-H292 IL-13 | 0.0 | 0.9 | 0.0 | 0.9 | 0.0 | 0.0 |
| NCI-H292 IFN | 0.0 | 0.5 | 0.0 | 0.6 | 0.0 | 0.0 |
| gamma | | | | | | |
| HPAEC none | 0.0 | 0.0 | 0.0 | 0.0 | 0.0 | 0.0 |
| HPAEC TNF alpha + | 0.0 | 0.0 | 0.0 | 0.0 | 0.0 | 0.0 |
| IL-1 beta | | | | | | |
| Lung fibroblast none | 31.4 | 95.9 | 0.2 | 65.5 | 94.0 | 54.3 |
| Lung fibroblast TNF | 22.2 | 48.6 | 0.1 | 39.8 | 62.9 | 52.5 |
| alpha + IL-1 beta | | | | | | |
| Lung fibroblast IL-4 | 19.1 | 27.4 | 0.1 | 21.2 | 34.9 | 0.0 |
| Lung fibroblast IL-9 | 23.5 | 24.0 | 0.1 | 26.8 | 96.6 | 66.0 |
| Lung fibroblast IL- | 4.5 | 11.9 | 0.0 | 10.4 | 13.4 | 0.0 |
| 13 | | | | | | |
| Lung fibroblast IFN | 15.7 | 55.9 | 0.2 | 46.3 | 89.5 | 47.0 |
| gamma | | | | | | |
| Dermal fibroblast | 0.0 | 6.0 | 0.0 | 6.3 | 4.1 | 0.0 |
| CCD1070 rest | | | | | | |
| Dermal fibroblast | 0.0 | 2.7 | 0.0 | 0.8 | 2.3 | 0.0 |
| CCD1070 TNF | | | | | | |
| alpha | | | | | | |
| Dermal fibroblast | 0.0 | 5.6 | 0.0 | 1.3 | 0.0 | 0.0 |
| CCD1070 IL-1 beta | | | | | | |
| Dermal fibroblast | 8.5 | 30.6 | 0.1 | 20.2 | 26.6 | 0.0 |
| IFN gamma | | | | | | |
| Dermal fibroblast | 4.1 | 30.8 | 0.1 | 19.8 | 25.5 | 0.0 |
| IL-4 | | | | | | |
| Dermal Fibroblasts | 8.0 | 54.3 | 0.1 | 46.7 | 47.3 | 0.0 |
| rest | | | | | | |
| Neutrophils | 0.0 | 0.9 | 0.0 | 0.4 | 0.0 | 0.0 |
| TNFa + LPS | | | | | | |
| Neutrophils rest | 0.0 | 0.0 | 0.0 | 0.3 | 0.0 | 0.0 |
| Colon | 4.0 | 4.6 | 0.0 | 9.5 | 8.4 | 0.0 |
| Lung | 0.0 | 2.8 | 0.0 | 4.6 | 2.1 | 0.0 |
| Thymus | 0.0 | 0.0 | 0.0 | 0.4 | 2.4 | 0.0 |
| Kidney | 4.9 | 7.8 | 0.1 | 9.7 | 5.2 | 0.0 |

TABLE AZO
general oncology screening panel_v_2.4

| | Rel. Exp. (%) |
|---|---|
| | Ag6442, Run |
| Tissue Name | 264979180 |
| Colon cancer 1 | 22.7 |
| Colon cancer NAT 1 | 100.0 |
| Colon cancer 2 | 0.0 |
| Colon cancer NAT 2 | 15.1 |
| Colon cancer 3 | 2.8 |
| Colon cancer NAT 3 | 40.1 |
| Colon malignant cancer 4 | 9.5 |
| Colon normal adjacent tissue 4 | 0.9 |
| Lung cancer 1 | 6.6 |
| Lung NAT 1 | 0.0 |
| Lung cancer 2 | 15.9 |
| Lung NAT 2 | 0.0 |
| Squamous cell carcinoma 3 | 8.3 |
| Lung NAT 3 | 0.0 |
| metastatic melanoma 1 | 49.0 |
| Melanoma 2 | 1.1 |
| Melanoma 3 | 13.8 |
| metastatic melanoma 4 | 24.0 |
| metastatic melanoma 5 | 31.4 |
| Bladder cancer 1 | 2.1 |
| Bladder cancer NAT 1 | 0.0 |
| Bladder cancer 2 | 19.3 |
| Bladder cancer NAT 2 | 1.4 |
| Bladder cancer NAT 3 | 4.8 |
| Bladder cancer NAT 4 | 66.0 |
| Prostate adenocarcinoma 1 | 7.5 |
| Prostate adenocarcinoma 2 | 8.0 |
| Prostate adenocarcinoma 3 | 9.0 |
| Prostate adenocarcinoma 4 | 9.1 |
| Prostate cancer NAT 5 | 9.9 |
| Prostate adenocarcinoma 6 | 7.7 |
| Prostate adenocarcinoma 7 | 17.3 |
| Prostate adenocarcinoma 8 | 0.0 |
| Prostate adenocarcinoma 9 | 33.9 |
| Prostate cancer NAT 10 | 4.9 |
| Kidney cancer 1 | 16.5 |
| KidneyNAT 1 | 7.2 |
| Kidney cancer 2 | 73.7 |
| Kidney NAT 2 | 19.2 |
| Kidney cancer 3 | 21.3 |
| Kidney NAT 3 | 11.4 |
| Kidney cancer 4 | 25.7 |
| Kidney NAT 4 | 14.9 |

CNS_neurodegeneration_v1.0

Summary: Ag6425/Ag6428/Ag6430/Ag6431/Ag6439/Ag6440/Ag6442 Seven experiments with different probe and primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. See Panel 1.4 for a discussion of this gene in treatment of central nervous system disorders.

Ag6424/Ag6391 Expression of this gene is low/undetectable (CTs>35) across all of the samples on this panel (data not shown).

General_screening_panel_v1.5 Summary: Ag6442 Highest expression of this gene is seen in skeletal muscle (CT=28). Expression of this gene is higher in adult (CT=28) as compared to the fetal skeletal muscle (CT=31). Therefore, expression of this gene may be used to distinguish fetal from adult skeletal muscle.

In addition moderate to low levels of expression of this gene is also seen in all the regions of central nervous system, in tissues with metabolic/endocrine functions and in a number of cancer cell lines derived from melanoma, brain, colon, lung, and ovarian cancers. This expression pattern is consistent with the expression seen in panel 1.4. See panel 1.4 for further discussion on the utility of these genes.

General_screening panel_v1.6 Summary: Ag6424/Ag6425/Ag6428/Ag6430/Ag6431/Ag6439/Ag6440/Ag6964 Nine experiments with seven different probe and primer sets are in very good agreement. Highest expression of this gene is detected in a ovarian cancer IGROV-1 cell line and brain cancer SNB-19 cell lines (CTs=25-33.7). In addition, consistent with expression seen in panel 1.4, moderate to low levels of expression of this gene is also seen in all the regions of central nervous system, tissues with metabolic/endocrine functions, and number of cancer cell lines. See panel 1.4 for further discussion of this gene.

Ag6391 Expression of this gene is low/undetectable (CTs>35) across all of the samples on this panel (data not shown).

Panel 4.1D Summary: Ag6425/Ag6428/Ag6430/Ag6431/Ag6439/Ag6440 Seven experiments with different probe and primer sets are in excellent agreement. Highest expression of this gene is detected in both resting and cytokine activated astrocytes (CTs=22-33.5). Therefore, therapeutic modulation of this gene or the design of therapeutics with the encoded protein could be important in the treatment of multiple sclerosis or other inflammatory diseases of the CNS.

In addition, moderate to low levels of expression of this gene is also seen in resting and cytokine treated lung and dermal fibroblasts, as well as in normal tissues represented by colon, lung, thymus and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Low levels of expression of this gene is also seen in liver cirrhosis. Therefore, antibodies or small molecule therapeutics could reduce or inhibit fibrosis that occurs in liver cirrhosis.

Ag6424 Expression of this gene is low/undetectable (CTs>35) across all of the samples on this panel (data not shown).

general oncology screening panel_v_{—}2.4 Summary: Ag6442 Two experiments with different probe and primer sets are in excellent agreement. Highest expression of this gene is seen in normal colon (CTs=29-32). Expression of this gene in normal colon is higher than in the corresponding cancer samples (CTs=32-34). Therefore, expression of this gene may be used to distinguish between these two samples.

Moderate expression of this gene is seen in both normal and cancer samples derived from colon, lung, bladder, prostate and kidney, as well as, in melanomas. Expression of this gene seems to be higher in kidney and lung cancers as compared to the corresponding normal adjacent samples. Therefore, expression of this gene may be used as marker to detect the presence of lung and kidney cancers. Furthermore, therapeutic modulation of this gene may be useful in the treatment of melanoma, colon, lung, bladder, prostate and kidney cancers.

BA. CG88634-01: KIAA1219-Like Protein.

Expression of gene CG88634-01 was assessed using the primer-probe set Ag3649, described in Table BAA. Results of the RTQ-PCR runs are shown in Tables BAB, BAC, BAD and BAE.

TABLE BAA

Probe Name Ag3649

| | | | Start | |
|---|---|---|---|---|
| Primers | Sequences | Length | Position | SEQ ID No |
| Forward | 5′-ccgcaagaattgaatcagtatc-3′ | 22 | 1055 | 768 |
| Probe | TET-5 -cctgccttaaacatctgcctcaaata-3′-TAMRA | 26 | 1077 | 769 |
| Reverse | 5′-catccaccagacagctgatt-3′ | 20 | 1123 | 770 |

TABLE BAB
CNS_neurodegeneration_v1.0

| | Rel. Exp. (%) |
|---|---|
| | Ag3649, Run |
| Tissue Name | 211019464 |
| AD 1 Hippo | 12.1 |
| AD 2 Hippo | 28.3 |
| AD 3 Hippo | 6.7 |
| AD 4 Hippo | 7.5 |
| AD 5 hippo | 100.0 |
| AD 6 Hippo | 47.6 |
| Control 2 Hippo | 25.2 |
| Control 4 Hippo | 9.2 |
| Control (Path) 3 Hippo | 7.5 |
| AD 1 Temporal Ctx | 18.6 |
| AD 2 Temporal Ctx | 30.4 |
| AD 3 Temporal Ctx | 5.9 |
| AD 4 Temporal Ctx | 24.5 |
| AD 5 Inf Temporal Ctx | 92.0 |
| AD 5 Sup Temporal Ctx | 48.0 |
| AD 6 Inf Temporal Ctx | 51.4 |
| AD 6 Sup Temporal Ctx | 47.6 |
| Control 1 Temporal Ctx | 6.9 |
| Control 2 Temporal Ctx | 28.1 |
| Control 3 Temporal Ctx | 12.9 |
| Control 4 Temporal Ctx | 6.9 |
| Control (Path) 1 Temporal Ctx | 57.0 |
| Control (Path) 2 Temporal Ctx | 36.3 |
| Control (Path) 3 Temporal Ctx | 4.5 |
| Control (Path) 4 Temporal Ctx | 40.9 |
| AD 1 Occipital Ctx | 12.2 |
| AD 2 Occipital Ctx (Missing) | 0.0 |
| AD 3 Occipital Ctx | 6.7 |
| AD 4 Occipital Ctx | 24.7 |
| AD 5 Occipital Ctx | 20.0 |
| AD 6 Occipital Ctx | 46.3 |
| Control 1 Occipital Ctx | 6.7 |
| Control 2 Occipital Ctx | 52.1 |
| Control 3 Occipital Ctx | 17.0 |
| Control 4 Occipital Ctx | 6.6 |
| Control (Path) 1 Occipital Ctx | 73.2 |
| Control (Path) 2 Occipital Ctx | 10.9 |
| Control (Path) 3 Occipital Ctx | 3.3 |
| Control (Path) 4 Occipital Ctx | 18.4 |
| Control 1 Parietal Ctx | 5.3 |
| Control 2 Parietal Ctx | 41.5 |
| Control 3 Parietal Ctx | 17.3 |
| Control (Path) 1 Parietal Ctx | 65.1 |
| Control (Path) 2 Parietal Ctx | 24.8 |
| Control (Path) 3 Parietal Ctx | 5.3 |
| Control (Path) 4 Parietal Ctx | 49.0 |

TABLE BAC
General_screening_panel_v1.4

| | Rel. Exp. (%) |
|---|---|
| | Ag3649, Run |
| Tissue Name | 219798089 |
| Adipose | 14.5 |
| Melanoma* Hs688(A).T | 31.9 |
| Melanoma* Hs688(B).T | 25.9 |
| Melanoma* M14 | 31.6 |
| Melanoma* LOXIMVI | 23.8 |
| Melanoma* SK-MEL-5 | 44.8 |
| Squamous cell carcinoma SCC-4 | 15.6 |
| Testis Pool | 19.9 |
| Prostate ca.* (bone met) PC-3 | 47.6 |
| Prostate Pool | 19.3 |
| Placenta | 21.5 |
| Uterus Pool | 12.3 |
| Ovarian ca. OVCAR-3 | 73.7 |
| Ovarian ca. SK-OV-3 | 51.8 |
| Ovarian ca. OVCAR-4 | 25.2 |
| Ovarian ca. OVCAR-5 | 44.1 |
| Ovarian ca. IGROV-1 | 18.8 |
| Ovarian ca. OVCAR-8 | 11.0 |
| Ovary | 15.5 |
| Breast ca. MCF-7 | 35.6 |
| Breast ca. MDA-MB-231 | 77.4 |
| Breast ca. BT 549 | 89.5 |
| Breast ca. T47D | 85.9 |
| Breast ca. MDA-N | 25.2 |
| Breast Pool | 27.0 |
| Trachea | 23.5 |
| Lung | 5.7 |
| Fetal Lung | 51.4 |
| Lung ca. NCI-N417 | 5.1 |
| Lung ca. LX-1 | 45.4 |
| Lung ca. NCI-H146 | 19.1 |
| Lung ca. SHP-77 | 28.5 |
| Lung ca. A549 | 19.9 |
| Lung ca. NCI-H526 | 12.0 |
| Lung ca. NCI-H23 | 64.6 |
| Lung ca. NCI-H460 | 19.2 |
| Lung ca. HOP-62 | 8.2 |
| Lung ca. NCI-H522 | 27.5 |
| Liver | 3.0 |
| Fetal Liver | 16.6 |
| Liver ca. HepG2 | 27.0 |
| Kidney Pool | 26.1 |
| Fetal Kidney | 34.2 |
| Renal ca. 786-0 | 25.5 |
| Renal ca. A498 | 12.7 |
| Renal ca. ACHN | 21.9 |
| Renal ca. UO-31 | 0.0 |
| Renal ca. TK-10 | 46.0 |
| Bladder | 26.4 |
| Gastric ca. (liver met.) NCI-N87 | 99.3 |
| Gastric ca. KATO III | 83.5 |
| Colon ca. SW-948 | 5.9 |
| Colon ca. SW480 | 63.7 |
| Colon ca.* (SW480 met) SW620 | 41.2 |
| Colon ca. HT29 | 20.6 |
| Colon ca. HCT-116 | 26.2 |
| Colon ca. CaCo-2 | 87.1 |
| Colon cancer tissue | 30.6 |
| Colon ca. SW1116 | 7.9 |
| Colon ca. Colo-205 | 7.0 |
| Colon ca. SW-48 | 8.8 |
| Colon Pool | 28.3 |
| Small Intestine Pool | 22.2 |
| Stomach Pool | 11.4 |
| Bone Marrow Pool | 16.0 |
| Fetal Heart | 26.8 |
| Heart Pool | 14.0 |
| Lymph Node Pool | 29.7 |
| Fetal Skeletal Muscle | 14.2 |
| Skeletal Muscle Pool | 13.5 |
| Spleen Pool | 17.0 |
| Thymus Pool | 25.0 |
| CNS cancer (glio/astro) U87-MG | 62.0 |
| CNS cancer (glio/astro) U-118-MG | 76.3 |
| CNS cancer (neuro; met) SK-N-AS | 63.7 |
| CNS cancer (astro) SF-539 | 30.8 |
| CNS cancer (astro) SNB-75 | 100.0 |
| CNS cancer (glio) SNB-19 | 18.8 |
| CNS cancer (glio) SF-295 | 84.1 |
| Brain (Amygdala) Pool | 10.6 |
| Brain (cerebellum) | 65.5 |
| Brain (fetal) | 36.3 |
| Brain (Hippocampus) Pool | 11.4 |
| Cerebral Cortex Pool | 19.9 |
| Brain (Substantia nigra) Pool | 10.0 |
| Brain (Thalamus) Pool | 22.4 |
| Brain (whole) | 20.9 |
| Spinal Cord Pool | 11.3 |
| Adrenal Gland | 25.3 |
| Pituitary gland Pool | 11.2 |
| Salivary Gland | 9.5 |
| Thyroid (female) | 6.0 |
| Pancreatic ca. CAPAN2 | 33.4 |
| Pancreas Pool | 28.1 |

TABLE BAD
Panel 4.1D

| | Rel. Exp. (%) |
|---|---|
| | Ag3649, Run |
| Tissue Name | 169975759 |
| Secondary Th1 act | 57.4 |
| Secondary Th2 act | 67.8 |
| Secondary Tr1 act | 81.8 |
| Secondary Th1 rest | 25.5 |
| Secondary Th2 rest | 47.6 |
| Secondary Tr1 rest | 41.8 |
| Primary Th1 act | 44.8 |
| Primary Th2 act | 54.3 |
| Primary Tr1 act | 49.7 |
| Primary Th1 rest | 36.3 |
| Primary Th2 rest | 41.5 |
| Primary Tr1 rest | 55.1 |
| CD45RA CD4 lymphocyte act | 38.4 |
| CD45RO CD4 lymphocyte act | 56.3 |
| CD8 lymphocyte act | 50.0 |
| Secondary CD8 lymphocyte rest | 51.4 |
| Secondary CD8 lymphocyte act | 29.9 |
| CD4 lymphocyte none | 42.0 |
| 2ry Th1/Th2/Tr1_anti-CD95 CH11 | 45.4 |
| LAK cells rest | 48.0 |
| LAK cells IL-2 | 54.7 |
| LAK cells IL-2 + IL-12 | 46.3 |
| LAK cells IL-2 + IFN gamma | 61.6 |
| LAK cells IL-2 + IL-18 | 65.5 |
| LAK cells PMA/ionomycin | 46.0 |
| NK Cells IL-2 rest | 65.1 |
| Two Way MLR 3 day | 64.6 |
| Two Way MLR 5 day | 45.1 |
| Two Way MLR 7 day | 30.4 |
| PBMC rest | 29.9 |
| PBMC PWM | 31.2 |
| PBMC PHA-L | 40.3 |
| Ramos (B cell) none | 47.3 |
| Ramos (B cell) ionomycin | 49.7 |
| B lymphocytes PWM | 28.5 |
| B lymphocytes CD40L and IL-4 | 55.1 |
| EOL-1 dbcAMP | 46.7 |
| EOL-1 dbcAMP PMA/ionomycin | 66.0 |
| Dendritic cells none | 44.8 |
| Dendritic cells LPS | 36.6 |
| Dendritic cells anti-CD40 | 53.2 |
| Monocytes rest | 58.2 |
| Monocytes LPS | 80.7 |
| Macrophages rest | 41.2 |
| Macrophages LPS | 28.7 |
| HUVEC none | 28.9 |
| HUVEC starved | 52.1 |
| HUVEC IL-1beta | 55.5 |
| HUVEC IFN gamma | 66.4 |
| HUVEC TNF alpha + IFN gamma | 50.0 |
| HUVEC TNF alpha + IL4 | 40.3 |
| HUVEC IL-11 | 32.3 |
| Lung Microvascular EC none | 96.6 |
| Lung Microvascular EC | 97.3 |
| TNFalpha + IL-1beta | |
| Microvascular Dermal EC none | 55.9 |
| Microsvasular Dermal EC | 60.3 |
| TNFalpha + IL-1beta | |
| Bronchial epithelium | 47.3 |
| TNFalpha + IL1beta | |
| Small airway epithelium none | 23.7 |
| Small airway epithelium | 45.1 |
| TNFalpha + IL-1beta | |
| Coronery artery SMC rest | 33.7 |
| Coronery artery SMC | 40.1 |
| TNFalpha + IL-1beta | |
| Astrocytes rest | 40.1 |
| Astrocytes TNFalpha + IL-1beta | 29.9 |
| KU-812 (Basophil) rest | 27.7 |
| KU-812 (Basophil) PMA/ionomycin | 48.0 |
| CCD1106 (Keratinocytes) none | 79.0 |
| CCD1106 (Keratinocytes) | 100.0 |
| TNFalpha + IL-1beta | |
| Liver cirrhosis | 17.2 |
| NC1-H292 none | 41.5 |
| NCI-H292 IL-4 | 71.7 |
| NCI-H292 IL-9 | 100.0 |
| NCI-H292 IL-13 | 75.3 |
| NCI-H292 IFN gamma | 72.2 |
| HPAEC none | 43.8 |
| HPAEC TNF alpha + IL-1 beta | 92.0 |
| Lung fibroblast none | 56.3 |
| Lung fibroblast TNF alpha + IL-1 beta | 27.9 |
| Lung fibroblast IL-4 | 39.8 |
| Lung fibroblast IL-9 | 53.6 |
| Lung fibroblast IL-13 | 31.4 |
| Lung fibroblast IFN gamma | 42.9 |
| Dermal fibroblast CCD1070 rest | 55.9 |
| Dermal fibroblast CCD1070 TNF alpha | 83.5 |
| Dermal fibroblast CCD1070 IL-1 beta | 36.1 |
| Dermal fibroblast IFN gamma | 34.9 |
| Dermal fibroblast IL-4 | 61.6 |
| Dermal Fibroblasts rest | 37.1 |
| Neutrophils TNFa + LPS | 14.3 |
| Neutrophils rest | 64.6 |
| Colon | 19.9 |
| Lung | 30.4 |
| Thymus | 85.3 |
| Kidney | 45.7 |

TABLE BAE
general oncology screening panel_v_2.4

| | Rel. Exp. (%) |
|---|---|
| | Ag3649, Run |
| Tissue Name | 267777885 |
| Colon cancer 1 | 30.4 |
| Colon cancer NAT 1 | 7.7 |
| Colon cancer 2 | 29.7 |
| Colon cancer NAT 2 | 10.7 |
| Colon cancer 3 | 76.3 |
| Colon cancer NAT 3 | 17.6 |
| Colon malignant cancer 4 | 52.9 |
| Colon normal adjacent tissue 4 | 20.6 |
| Lung cancer 1 | 20.3 |
| Lung NAT 1 | 5.1 |
| Lung cancer 2 | 88.9 |
| Lung NAT 2 | 9.5 |
| Squamous cell carcinoma 3 | 39.8 |
| Lung NAT 3 | 4.4 |
| metastatic melanoma 1 | 31.6 |
| Melanoma 2 | 5.6 |
| Melanoma 3 | 4.8 |
| metastatic melanoma 4 | 46.7 |
| metastatic melanoma 5 | 84.7 |
| Bladder cancer 1 | 3.4 |
| Bladder cancer NAT 1 | 0.0 |
| Bladder cancer 2 | 23.7 |
| Bladder cancer NAT 2 | 2.0 |
| Bladder cancer NAT 3 | 4.8 |
| Bladder cancer NAT 4 | 14.8 |
| Prostate adenocarcinoma 1 | 65.5 |
| Prostate adenocarcinoma 2 | 11.0 |
| Prostate adenocarcinoma 3 | 36.6 |
| Prostate adenocarcinoma 4 | 38.4 |
| Prostate cancer NAT 5 | 12.4 |
| Prostate adenocarcinoma 6 | 16.4 |
| Prostate adenocarcinoma 7 | 20.6 |
| Prostate adenocarcinoma 8 | 13.6 |
| Prostate adenocarcinoma 9 | 62.9 |
| Prostate cancer NAT 10 | 10.9 |
| Kidney cancer 1 | 39.5 |
| Kidney NAT 1 | 23.8 |
| Kidney cancer 2 | 100.0 |
| Kidney NAT 2 | 31.6 |
| Kidney cancer 3 | 33.9 |
| Kidney NAT 3 | 8.5 |
| Kidney cancer 4 | 13.5 |
| Kidney NAT 4 | 6.3 |

CNS_neurodegeneration_v1.0 Summary: Ag3649 This panel does not show differential expression of this gene in Alzheimer's disease. However, this profile confirms the expression of this gene at moderate levels in the brain. See Panel 1.4 for discussion of this gene in the central nervous system.

General_screening_panel_v1.4 Summary: Ag3649 Highest expression of this gene is seen in a brain cancer cell line (CT=25). This gene is widely expressed in this panel, with high levels of expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at high to moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

In addition, this gene is expressed at much higher levels in fetal lung tissue (CT=26) when compared to expression in the adult counterpart (CT=29). Thus, expression of this gene may be used to differentiate between the fetal and adult source of this tissue.

This gene is also expressed at high to moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

Panel 4.1D Summary: Ag3649 Highest expression of this gene is seen in IL-9 treated NCI-H292 cells and TNF-a and IL-1b treated keratinocytes (CT=27.3). This gene is also expressed at hight to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

general oncology screening panel_v_{—}2.4 Summary: Ag3649 Highest expression of this gene is detected in kidney cancer (CT=27.6). Significant expression of this gene is detected both in normal and cancer samples derived from colon, kidney, bladder, lung, prostate and melanoma. Expression of this gene is higher in cancer samples as compared to the corresponding normal adjacent samples. Therefore, expression of this gene may be use as diagnostic marker for lung, colon, prostate, kidney and bladder cancer, as well as metastatic melanoma. In addition, therapeutic modulation of this gene through the use of antibodoy or small molecule drug may be beneficial in the treatment of melenoma, prostate, lung, colon, kidney and bladder cancers.

BB. CG97012-01 and CG97012-02: Seizure 6 Precursor Protein-Like Protein.

Expression of gene CG97012-01 and CG97012-02 was assessed using the primer-probe sets Ag1477 and Ag4105, described in Tables BBA and BBB. Results of the RTQ-PCR runs are shown in Tables BBC, BBD, BBE, BBF, BBG, BBH, BBI and BBJ.

TABLE BBA

Probe Name Ag 1477

| | | | Start | |
|---|---|---|---|---|
| Primers | Sequences | Length | Position | SEQ ID No |
| Forward | 5′-aatcctgaggggtacattgact-3′ | 22 | 859 | 771 |
| Probe | TET-5′-ccctcaacaactttctggagtggcaca-3′-TAMRA | 26 | 902 | 772 |
| Reverse | 5′-agccagttgtagactgtcacgtt-3′ | 22 | 931 | 773 |

TABLE BBB

Probe Name Ag4105

| | | | Start | |
|---|---|---|---|---|
| Primers | Sequences | Length | Position | SEQ ID No |
| Forward | 5′-aatcctgaggggtacattgact-3′ | 22 | 859 | 774 |
| Probe | TET-5′-ccctcaacaactttctggagtgcaca-3′-TAMRA | 26 | 902 | 775 |
| Reverse | 5′-agccagtgtagactgtcacgtt-3′ | 22 | 931 | 776 |

TABLE BBC
AI_comprehensive panel_v1.0

| | Rel. Exp. (%) |
|---|---|
| | Ag4105, |
| | Run |
| Tissue Name | 255325336 |
| 110967 COPD-F | 7.7 |
| 110980 COPD-F | 1.9 |
| 110968 COPD-M | 0.0 |
| 110977 COPD-M | 4.4 |
| 110989 Emphysema-F | 9.6 |
| 110992 Emphysema-F | 1.6 |
| 110993 Emphysema-F | 13.8 |
| 110994 Emphysema-F | 0.0 |
| 110995 Emphysema-F | 0.0 |
| 110996 Emphysema-F | 0.0 |
| 110997 Asthma-M | 4.5 |
| 111001 Asthma-F | 0.0 |
| 111002 Asthma-F | 1.8 |
| 111003 Atopic Asthma-F | 5.8 |
| 111004 Atopic Asthma-F | 6.9 |
| 111005 Atopic Asthma-F | 3.1 |
| 111006 Atopic Asthma-F | 0.0 |
| 111417 Allergy-M | 0.0 |
| 112347 Allergy-M | 15.2 |
| 112349 Normal Lung-F | 11.0 |
| 112357 Normal Lung-F | 2.2 |
| 112354 Normal Lung-M | 7.1 |
| 112374 Crohns-F | 25.0 |
| 112389 Match Control Crohns-F | 0.9 |
| 112375 Crohns-F | 8.3 |
| 112732 Match Control Crohns-F | 3.3 |
| 112725 Crohns-M | 7.4 |
| 112387 Match Control Crohns-M | 2.7 |
| 112378 Crohns-M | 20.9 |
| 112390 Match Control Crohns-M | 6.9 |
| 112726 Crohns-M | 15.3 |
| 112731 Match Control Crohns-M | 5.9 |
| 112380 Ulcer Col-F | 2.1 |
| 112734 Match Control Ulcer Col-F | 2.0 |
| 112384 Ulcer Col-F | 2.1 |
| 112737 Match Control Ulcer Col-F | 1.4 |
| 112386 Ulcer Col-F | 4.2 |
| 112738 Match Control Ulcer Col-F | 1.9 |
| 112381 Ulcer Col-M | 5.8 |
| 112735 Match Control Ulcer Col-M | 39.0 |
| 112382 Ulcer Col-M | 11.1 |
| 112394 Match Control Ulcer Col-M | 2.0 |
| 112383 Ulcer Col-M | 4.9 |
| 112736 Match Control Ulcer Col-M | 0.0 |
| 112423 Psoriasis-F | 5.5 |
| 112427 Match Control Psoriasis-F | 1.3 |
| 112418 Psoriasis-M | 1.1 |
| 112723 Match Control Psoriasis-M | 3.9 |
| 112419 Psoriasis-M | 0.0 |
| 112424 Match Control Psoriasis-M | 1.2 |
| 112420 Psoriasis-M | 8.2 |
| 112425 Match Control Psoriasis-M | 5.8 |
| 104689 (MF) OA Bone-Backus | 6.7 |
| 104690 (MF) Adj “Normal” Bone-Backus | 1.9 |
| 104691 (MF) OA Synovium-Backus | 49.3 |
| 104692 (BA) OA Cartilage-Backus | 36.6 |
| 104694 (BA) OA Bone-Backus | 7.9 |
| 104695 (BA) Adj “Normal” Bone-Backus | 3.7 |
| 104696 (BA) OA Synovium-Backus | 22.1 |
| 104700 (SS) OA Bone-Backus | 4.2 |
| 104701 (SS) Adj “Normal” Bone-Backus | 6.6 |
| 104702 (SS) OA Synovium-Backus | 15.1 |
| 117093 OA Cartilage Rep7 | 5.8 |
| 112672 OA Bone5 | 3.7 |
| 112673 OA Synovium5 | 0.0 |
| 112674 OA Synovial Fluid cells5 | 1.7 |
| 117100 OA Cartilage Rep 14 | 0.0 |
| 112756 OA Bone9 | 100.0 |
| 112757 OA Synovium9 | 6.0 |
| 112758 OA Synovial Fluid Cells9 | 6.3 |
| 117125 RA Cartilage Rep2 | 1.8 |
| 113492 Bone2 RA | 14.8 |
| 113493 Synovium2 RA | 3.0 |
| 113494 Syn Fluid Cells RA | 11.6 |
| 113499 Cartilage4 RA | 10.7 |
| 113500 Bone4 RA | 7.1 |
| 113501 Synovium4 RA | 5.4 |
| 113502 Syn Fluid Cells4 RA | 2.0 |
| 113495 Cartilage3 RA | 2.0 |
| 113496 Bone3 RA | 14.5 |
| 113497 Synovium3 RA | 6.8 |
| 113498 Syn Fluid Cells3 RA | 10.5 |
| 117106 Normal Cartilage Rep20 | 0.0 |
| 113663 Bone3 Normal | 8.3 |
| 113664 Synovium3 Normal | 8.8 |
| 113665 Syn Fluid Cells3 Normal | 8.2 |
| 117107 Normal Cartilage Rep22 | 2.3 |
| 113667 Bone4 Normal | 9.4 |
| 113668 Synovium4 Normal | 4.7 |
| 113669 Syn Fluid Cells4 Normal | 0.0 |

TABLE BBD
Ardais Panel v.1.0

| | Rel. Exp. (%), |
|---|---|
| | Ag1477, |
| | Run |
| Tissue Name | 263245998 |
| 136799_Lung cancer(362) | 5.4 |
| 136800_Lung NAT(363) | 49.3 |
| 136813_Lung cancer(372) | 5.4 |
| 136814_Lung NAT(373) | 6.2 |
| 136815_Lung cancer(374) | 18.0 |
| 136816_Lung NAT(375) | 62.4 |
| 136791_Lung cancer(35A) | 3.8 |
| 136795_Lung cancer(35E) | 1.9 |
| 136797_Lung cancer(360) | 0.0 |
| 136794_lung NAT(35D) | 24.5 |
| 136818_Lung NAT(377) | 33.2 |
| 136787_lung cancer(356) | 2.4 |
| 136788_lung NAT(357) | 17.8 |
| 136804_Lung cancer(369) | 9.9 |
| 136805_Lung NAT(36A) | 12.8 |
| 136806_Lung cancer(36B) | 1.7 |
| 136807_Lung NAT(36C) | 19.6 |
| 136789_lung cancer(358) | 15.0 |
| 136802_Lung cancer(365) | 21.2 |
| 136803_Lung cancer(368) | 7.7 |
| 136811_Lung cancer(370) | 5.1 |
| 136810_Lung NAT(36F) | 100.0 |

TABLE BBE
CNS_neurodegeneration v1.0

| | Rel. Exp. (%) | Rel. Exp. (%) |
|---|---|---|
| | Ag1477, | Ag4105, |
| | Run | Run |
| Tissue Name | 206941434 | 206943848 |
| AD 1 Hippo | 11.9 | 10.6 |
| AD 2 Hippo | 33.7 | 23.3 |
| AD 3 Hippo | 8.1 | 7.0 |
| AD 4 Hippo | 5.1 | 5.0 |
| AD 5 hippo | 100.0 | 100.0 |
| AD 6 Hippo | 49.7 | 40.9 |
| Control 2 Hippo | 61.1 | 47.0 |
| Control 4 Hippo | 4.1 | 3.8 |
| Control (Path) 3 Hippo | 3.7 | 3.3 |
| AD 1 Temporal Ctx | 6.1 | 4.7 |
| AD 2 Temporal Ctx | 27.0 | 23.8 |
| AD 3 Temporal Ctx | 3.1 | 2.6 |
| AD 4 Temporal Ctx | 14.4 | 10.3 |
| AD 5 Inf Temporal Ctx | 66.9 | 71.2 |
| AD 5 Sup Temporal Ctx | 36.1 | 32.3 |
| AD 6 Inf Temporal Ctx | 25.7 | 25.3 |
| AD 6 Sup Temporal Ctx | 27.4 | 22.5 |
| Control 1 Temporal Ctx | 4.5 | 3.9 |
| Control 2 Temporal Ctx | 52.9 | 42.0 |
| Control 3 Temporal Ctx | 14.8 | 11.4 |
| Control 4 Temporal Ctx | 4.9 | 4.4 |
| Control (Path) 1 Temporal Ctx | 69.3 | 57.8 |
| Control (Path) 2 Temporal Ctx | 33.0 | 23.7 |
| Control (Path) 3 Temporal Ctx | 2.6 | 2.6 |
| Control (Path) 4 Temporal Ctx | 29.3 | 17.9 |
| AD 1 Occipital Ctx | 7.9 | 7.7 |
| AD 2 Occipital Ctx (Missing) | 0.0 | 0.0 |
| AD 3 Occipital Ctx | 3.9 | 3.2 |
| AD 4 Occipital Ctx | 17.2 | 10.5 |
| AD 5 Occipital Ctx | 15.1 | 55.5 |
| AD 6 Occipital Ctx | 69.3 | 14.1 |
| Control 1 Occipital Ctx | 1.2 | 0.8 |
| Control 2 Occipital Ctx | 88.3 | 84.7 |
| Control 3 Occipital Ctx | 16.2 | 13.4 |
| Control 4 Occipital Ctx | 2.3 | 2.3 |
| Control (Path) 1 Occipital Ctx | 92.7 | 73.7 |
| Control (Path) 2 Occipital Ctx | 5.4 | 7.0 |
| Control (Path) 3 Occipital Ctx | 1.1 | 0.4 |
| Control (Path) 4 Occipital Ctx | 14.1 | 10.6 |
| Control 1 Parietal Ctx | 3.9 | 2.2 |
| Control 2 Parietal Ctx | 15.8 | 19.2 |
| Control 3 Parietal Ctx | 17.4 | 14.9 |
| Control (Path) 1 Parietal Ctx | 99.3 | 92.7 |
| Control (Path) 2 Parietal Ctx | 23.7 | 16.0 |
| Control (Path) 3 Parietal Ctx | 2.1 | 2.7 |
| Control (Path) 4 Parietal Ctx | 58.2 | 29.5 |

TABLE BBF
General_screening_panel_v1.4

| | Rel. Exp. (%) | Rel. Exp. (%) |
|---|---|---|
| | Ag1477, | Ag4105, |
| | Run | Run |
| Tissue Name | 213323518 | 212714156 |
| Adipose | 0.2 | 0.1 |
| Melanoma* Hs688(A).T | 0.0 | 0.0 |
| Melanoma* Hs688(B).T | 0.0 | 0.0 |
| Melanoma* M14 | 0.0 | 0.0 |
| Melanoma*LOXIMVI | 0.0 | 0.0 |
| Melanoma* SK-MEL-5 | 0.0 | 0.0 |
| Squamous cell carcinoma SCC-4 | 0.0 | 0.0 |
| Testis Pool | 0.2 | 0.1 |
| Prostate ca.* (bone met) PC-3 | 0.0 | 0.0 |
| Prostate Pool | 0.2 | 0.1 |
| Placenta | 0.0 | 0.0 |
| Uterus Pool | 0.0 | 0.0 |
| Ovarian ca. OVCAR-3 | 0.0 | 0.0 |
| Ovarian ca. SK-OV-3 | 6.5 | 0.0 |
| Ovarian ca. OVCAR-4 | 0.0 | 0.0 |
| Ovarian ca. OVCAR-5 | 0.0 | 0.0 |
| Ovarian ca. IGROV-1 | 0.0 | 0.0 |
| Ovarian ca. OVCAR-8 | 0.0 | 0.0 |
| Ovary | 0.1 | 0.1 |
| Breast ca. MCF-7 | 0.0 | 0.0 |
| Breast ca. MDA-MB-231 | 0.0 | 0.0 |
| Breast ca. BT 549 | 0.0 | 0.0 |
| Breast ca. T47D | 0.0 | 0.1 |
| Breast ca. MDA-N | 0.0 | 0.0 |
| Breast Pool | 0.0 | 0.1 |
| Trachea | 0.2 | 0.3 |
| Lung | 0.0 | 0.0 |
| Fetal Lung | 0.6 | 0.3 |
| Lung ca. NCI-N417 | 30.1 | 11.4 |
| Lung ca. LX-1 | 0.0 | 0.0 |
| Lung ca. NCI-H146 | 22.2 | 18.3 |
| Lung ca. SHP-77 | 9.0 | 4.3 |
| Lung ca. A549 | 0.0 | 0.0 |
| Lung ca. NCI-H526 | 11.0 | 4.3 |
| Lung ca. NCI-H23 | 1.3 | 0.7 |
| Lung ca. NCI-H460 | 0.2 | 0.1 |
| Lung ca. HOP-62 | 0.0 | 0.0 |
| Lung ca. NCI-H522 | 0.0 | 0.0 |
| Liver | 0.0 | 0.0 |
| Fetal Liver | 0.1 | 0.2 |
| Liver ca. HepG2 | 0.0 | 0.0 |
| Kidney Pool | 0.1 | 0.0 |
| Fetal Kidney | 0.1 | 0.0 |
| Renal ca. 786-0 | 0.0 | 0.0 |
| Renal ca. A498 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Bladder | 0.6 | 0.3 |
| Gastric ca. (liver met.) NCI-N87 | 0.0 | 0.0 |
| Gastric ca. KATO III | 0.0 | 0.0 |
| Colon ca. SW-948 | 0.0 | 0.0 |
| Colon ca. SW480 | 0.0 | 0.0 |
| Colon ca.* (SW480 met) SW620 | 0.0 | 0.0 |
| Colon ca. HT29 | 0.0 | 0.0 |
| Colon ca. HCT-116 | 0.0 | 0.0 |
| Colon ca. CaCo-2 | 0.0 | 0.0 |
| Colon cancer tissue | 0.0 | 0.0 |
| Colon ca. SW1116 | 0.0 | 0.0 |
| Colon ca. Colo-205 | 0.0 | 0.0 |
| Colon ca. SW-48 | 0.0 | 0.0 |
| Colon Pool | 0.1 | 0.0 |
| Small Intestine Pool | 0.4 | 0.2 |
| Stomach Pool | 6.0 | 0.1 |
| Bone Marrow Pool | 0.1 | 0.1 |
| Fetal Heart | 0.1 | 0.0 |
| Heart Pool | 0.1 | 0.0 |
| Lymph Node Pool | 0.3 | 0.1 |
| Fetal Skeletal Muscle | 0.2 | 0.0 |
| Skeletal Muscle Pool | 0.0 | 0.0 |
| Spleen Pool | 0.1 | 0.1 |
| Thymus Pool | 0.2 | 0.2 |
| CNS cancer (glio/astro) U87-MG | 0.0 | 0.0 |
| CNS cancer (glio/astro) U-118-MG | 1.2 | 1.0 |
| CNS cancer (neuro; met) SK-N-AS | 0.0 | 0.0 |
| CNS cancer (astro) SF-539 | 0.0 | 0.0 |
| CNS cancer (astro) SNB-75 | 0.3 | 0.1 |
| CNS cancer (glio) SNB-19 | 0.0 | 0.0 |
| CNS cancer (glio) SF-295 | 0.0 | 0.0 |
| Brain (Amygdala) Pool | 21.8 | 9.3 |
| Brain (cerebellum) | 87.1 | 54.3 |
| Brain (fetal) | 100.0 | 100.0 |
| Brain (Hippocampus) Pool | 28.9 | 25.5 |
| Cerebral Cortex Pool | 40.9 | 15.9 |
| Brain (Substantia nigra) Pool | 29.3 | 12.1 |
| Brain (Thalamus) Pool | 37.9 | 14.7 |
| Brain (whole) | 43.5 | 26.4 |
| Spinal Cord Pool | 7.5 | 4.0 |
| Adrenal Gland | 4.3 | 1.5 |
| Pituitary gland Pool | 2.9 | 2.9 |
| Salivary Gland | 0.2 | 0.1 |
| Thyroid (female) | 0.1 | 0.2 |
| Pancreatic ca. CAPAN2 | 0.0 | 0.0 |
| Pancreas Pool | 0.6 | 0.2 |

TABLE BBG
Panel 3D

| | Rel. Exp. (%) |
|---|---|
| | Ag1477, |
| | Run |
| Tissue Name | 215538905 |
| Daoy- Medulloblastoma | 0.0 |
| TE671- Medulloblastoma | 0.0 |
| D283 Med-Medulloblastoma | 0.0 |
| PFSK-1- Primitive Neuroectodermal | 0.0 |
| XF-498- CNS | 0.7 |
| SNB-78- Glioma | 0.0 |
| SF-268- Glioblastoma | 0.0 |
| T98G- Glioblastoma | 0.0 |
| SK-N-SH- Neuroblastoma (metastasis) | 3.8 |
| SF-295- Glioblastoma | 0.0 |
| Cerebellum | 34.6 |
| Cerebellum | 50.7 |
| NCI-H292- Mucoepidermoid lung carcinoma | 0.3 |
| DMS-114- Small cell lung cancer | 0.6 |
| DMS-79- Small cell lung cancer | 100.0 |
| NCI-H146- Small cell lung cancer | 61.1 |
| NCI-H526- Small cell lung cancer | 49.0 |
| NCI-N417- Small cell lung cancer | 78.5 |
| NCI-H82- Small cell lung cancer | 23.7 |
| NCI-H157- Squamous cell lung cancer (metastasis) | 0.0 |
| NCI-H1155- Large cell lung cancer | 18.8 |
| NCI-H1299- Large cell lung cancer | 0.0 |
| NCI-H727- Lung carcinoid | 1.7 |
| NCI-UMC-11- Lung carcinoid | 0.0 |
| LX-1- Small cell lung cancer | 0.0 |
| Colo-205- Colon cancer | 0.0 |
| KM12- Colon cancer | 0.1 |
| KM20L2- Colon cancer | 0.0 |
| NCI-H716- Colon cancer | 0.0 |
| SW-48- Colon adenocarcinoma | 0.0 |
| SW1116- Colon adenocarcinoma | 0.0 |
| LS 174T- Colon adenocarcinoma | 0.0 |
| SW-948- Colon adenocarcinoma | 0.0 |
| SW-480- Colon adenocarcinoma | 0.0 |
| NCI-SNU-5- Gastric carcinoma | 0.0 |
| KATO III- Gastric carcinoma | 0.0 |
| NCI-SNU-16- Gastric carcinoma | 0.0 |
| NCI-SNU-1- Gastric carcinoma | 0.0 |
| RF-1- Gastric adenocarcinoma | 0.2 |
| RF-48- Gastric adenocarcinoma | 0.0 |
| MKN-45- Gastric carcinoma | 2.7 |
| NCI-N87- Gastric carcinoma | 0.0 |
| OVCAR-5- Ovarian carcinoma | 0.0 |
| RL95-2- Uterine carcinoma | 0.0 |
| HelaS3- Cervical adenocarcinoma | 0.0 |
| Ca Ski- Cervical epidermoid carcinoma | 0.0 |
| (metastasis) | |
| ES-2- Ovarian clear cell carcinoma | 0.0 |
| Ramos- Stimulated with PMA/ionomycin 6 h | 0.0 |
| Ramos- Stimulated with PMA/ionomycin 14 h | 0.0 |
| MEG-01- Chronic myelogenous leukemia | 0.0 |
| (megokaryoblast) | |
| Raji- Burkitt's lymphoma | 0.0 |
| Daudi- Burkitt's lymphoma | 0.0 |
| U266- B-cell plasmacytoma | 0.0 |
| CA46- Burkitt's lymphoma | 0.0 |
| RL- non-Hodgkin's B-cell lymphoma | 0.0 |
| JM1- pre-B-cell lymphoma | 0.0 |
| Jurkat- T cell leukemia | 0.0 |
| TF-1- Erythroleukemia | 0.0 |
| HUT 78- T-cell lymphoma | 0.0 |
| U937- Histiocytic lymphoma | 0.0 |
| KU-812- Myelogenous leukemia | 0.0 |
| 769-P- Clear cell renal carcinoma | 0.0 |
| Caki-2- Clear cell renal carcinoma | 0.0 |
| SW 839- Clear cell renal carcinoma | 0.0 |
| G401- Wilms' tumor | 0.0 |
| Hs766T- Pancreatic carcinoma (LN metastasis) | 0.0 |
| CAPAN-1 -Pancreatic | 0.0 |
| adenocarcinoma (liver metastasis) | |
| SU86.86- Pancreatic | 0.0 |
| carcinoma (liver metastasis) | |
| BxPC-3- Pancreatic adenocarcinoma | 0.0 |
| HPAC- Pancreatic adenocarcinoma | 0.0 |
| MIA PaCa-2- Pancreatic carcinoma | 0.0 |
| CFPAC-1- Pancreatic ductal adenocarcinoma | 0.0 |
| PANC-1- Pancreatic | 0.0 |
| epithelioid ductal carcinoma | |
| T24- Bladder carcinma (transitional cell) | 0.0 |
| 5637- Bladder carcinoma | 0.0 |
| HT-1197- Bladder carcinoma | 0.0 |
| UM-UC-3- Bladder carcinma (transitional cell) | 0.0 |
| A204- Rhabdomyosarcoma | 0.0 |
| HT-1080- Fibrosarcoma | 0.0 |
| MG-63- Osteosarcoma | 0.0 |
| SK-LMS-1- Leiomyosarcoma (vulva) | 0.0 |
| SJRH30- Rhabdomyosarcoma (met to bone marrow) | 0.0 |
| A431- Epidermoid carcinoma | 0.0 |
| WM266-4- Melanoma | 0.0 |
| DU145- Prostate carcinoma (brain metastasis) | 0.0 |
| MDA-MB-468- Breast adenocarcinoma | 0.0 |
| SCC-4- Squamous cell carcinoma of tongue | 0.0 |
| SCC-9- Squamous cell carcinoma of tongue | 0.0 |
| SCC-15- Squamous cell carcinoma of tongue | 0.0 |
| CAL 27- Squamous cell carcinoma of tongue | 0.0 |

TABLE BBH
Panel 4.1D

| | Rel. Exp. (%) | Rel. Exp. (%) |
|---|---|---|
| | Ag1477, | Ag4105, |
| | Run | Run |
| Tissue Name | 200923970 | 175180105 |
| Secondary Th1 act | 0.0 | 0.0 |
| Secondary Th2 act | 0.0 | 0.0 |
| Secondary Tr1 act | 4.5 | 0.0 |
| Secondary Th1 rest | 0.0 | 0.0 |
| Secondary Th2 rest | 3.3 | 0.0 |
| Secondary Tr1 rest | 0.0 | 0.0 |
| Primary Th1 act | 0.0 | 0.0 |
| Primary Th2 act | 0.0 | 0.0 |
| Primary Tr1 act | 1.5 | 0.0 |
| Primary Th1 rest | 0.0 | 0.0 |
| Primary Th2 rest | 0.0 | 0.0 |
| Primary Tr1 rest | 0.0 | 0.0 |
| CD45RA CD4 lymphocyte act | 0.0 | 4.0 |
| CD45RO CD4 lymphocyte act | 0.0 | 0.0 |
| CD8 lymphocyte act | 0.0 | 3.9 |
| Secondary CD8 lymphocyte rest | 0.0 | 1.4 |
| Secondary CD8 lymphocyte act | 0.0 | 0.0 |
| CD4 lymphocyte none | 5.6 | 2.6 |
| 2ry Th1/Th2/Tr1 anti-CD95 CH11 | 2.6 | 1.4 |
| LAK cells rest | 0.0 | 2.8 |
| LAK cells IL-2 | 4.1 | 1.1 |
| LAK cells IL-2 + IL-12 | 0.0 | 0.0 |
| LAK cells IL-2 + IFN gamma | 0.0 | 0.0 |
| LAK cells IL-2 + IL-18 | 0.0 | 0.0 |
| LAK cells PMA/ionomycin | 0.0 | 0.0 |
| NK Cells IL-2 rest | 11.7 | 0.0 |
| Two Way MLR 3 day | 4.7 | 0.0 |
| Two Way MLR 5 day | 0.0 | 0.0 |
| Two Way MLR 7 day | 0.0 | 0.0 |
| PBMC rest | 26.8 | 8.5 |
| PBMC PWM | 0.0 | 0.0 |
| PBMC PHA-L | 0.0 | 0.0 |
| Ramos (B cell) none | 0.0 | 0.0 |
| Ramos (B cell) ionomycin | 0.0 | 0.0 |
| B lymphocytes PWM | 0.0 | 0.0 |
| B lymphocytes CD40L and IL-4 | 0.0 | 0.0 |
| EOL-1 dbcAMP | 0.0 | 0.0 |
| EOL-1 dbcAMP PMA/ionomycin | 0.0 | 0.0 |
| Dendritic cells none | 35.1 | 11.5 |
| Dendritic cells LPS | 9.4 | 2.3 |
| Dendritic cells anti-CD40 | 24.5 | 14.1 |
| Monocytes rest | 93.3 | 37.6 |
| Monocytes LPS | 5.6 | 2.5 |
| Macrophages rest | 0.0 | 0.0 |
| Macrophages LPS | 1.7 | 0.0 |
| HUVEC none | 0.0 | 0.0 |
| HUVEC starved | 0.0 | 0.0 |
| HUVEC IL-1beta | 0.0 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 0.0 | 0.9 |
| Lung Microvascular EC none | 0.0 | 7.0 |
| Lung Microvascular | 0.0 | 0.0 |
| EC TNFalpha + IL-1beta | | |
| Microvascular Dermal EC none | 0.0 | 0.0 |
| Microsvasular Dermal | 0.0 | 0.0 |
| EC TNFalpha + IL-1beta | | |
| Bronchial epithelium | 0.0 | 0.0 |
| TNFalpha + IL1beta | | |
| Small airway epithelium none | 0.0 | 0.0 |
| Small airway epithelium TNFalpha + | 0.0 | 0.0 |
| IL-1beta | | |
| Coronery artery SMC rest | 0.0 | 0.0 |
| Coronery artery SMC | 0.0 | 0.0 |
| TNFalpha + IL-1beta | | |
| Astrocytes rest | 4.1 | 0.0 |
| Astrocytes TNFalpha + IL-1beta | 0.0 | 0.0 |
| KU-8l2 (Basophil) rest | 0.0 | 0.0 |
| KU-812 (Basophil) PMA/ionomycin | 0.0 | 0.0 |
| CCD1106 (Keratinocytes) none | 0.0 | 0.0 |
| CCD1106 (Keratinocytes) | 0.0 | 0.0 |
| TNFalpha + IL-1beta | | |
| Liver cirrhosis | 0.0 | 0.0 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NC1-H292 IFN gamma | 0.0 | 0.0 |
| HPAEC none | 0.0 | 0.0 |
| HPAEC TNF alpha + IL-1 beta | 0.0 | 0.0 |
| Lung fibroblast none | 0.0 | 0.0 |
| Lung fibroblast | 0.0 | 0.0 |
| TNF alpha + IL-1 beta | | |
| Lung fibroblast IL-4 | 0.0 | 0.0 |
| Lung fibroblast IL-9 | 7.2 | 0.0 |
| Lung fibroblast IL-13 | 0.0 | 2.2 |
| Lung fibroblast IFN gamma | 0.0 | 0.0 |
| Dermal fibroblast CCD1070 rest | 0.0 | 0.6 |
| Dermal fibroblast CCD1070 TNF alpha | 0.0 | 0.0 |
| Dermal fibroblast CCD1070 IL-1 beta | 0.0 | 0.0 |
| Dermal fibroblast IFN gamma | 3.5 | 0.0 |
| Dermal fibroblast IL-4 | 0.0 | 0.0 |
| Dermal Fibroblasts rest | 1.6 | 0.0 |
| Neutrophils TNFa + LPS | 7.5 | 0.0 |
| Neutrophils rest | 3.8 | 3.1 |
| Colon | 17.6 | 5.8 |
| Lung | 1.2 | 4.5 |
| Thymus | 49.3 | 13.8 |
| Kidney | 100.0 | 100.0 |

TABLE BBI
Panel CNS_1.1

| | Rel. Exp. (%) |
|---|---|
| | Ag1477, |
| | Run |
| Tissue Name | 204172546 |
| Cing Gyr Depression2 | 4.2 |
| Cing Gyr Depression | 2.3 |
| Cing Gyr PSP2 | 1.3 |
| Cing Gyr PSP | 2.9 |
| Cing Gyr Huntington's2 | 4.4 |
| Cing Gyr Huntington's | 27.5 |
| Cing Gyr Parkinson's2 | 10.4 |
| Cing Gyr Parkinson's | 9.8 |
| Cing Gyr Alzheimer's2 | 3.4 |
| Cing Gyr Alzheimer's | 9.6 |
| Cing Gyr Control2 | 19.8 |
| Cing Gyr Control | 24.1 |
| Temp Pole Depression2 | 2.4 |
| Temp Pole PSP2 | 2.9 |
| Temp Pole PSP | 0.9 |
| Temp Pole Huntington's | 13.6 |
| Temp Pole Parkinson's2 | 9.4 |
| Temp Pole Parkinson's | 7.2 |
| Temp Pole Alzheimer's2 | 1.3 |
| Temp Pole Alzheimer's | 2.2 |
| Temp Pole Control2 | 15.8 |
| Temp Pole Control | 6.2 |
| Glob Palladus Depression | 0.8 |
| Glob Palladus PSP2 | 1.5 |
| Glob Palladus PSP | 0.5 |
| Glob Palladus Parkinson's2 | 0.8 |
| Glob Palladus Parkinson's | 13.4 |
| Glob Palladus Alzheimer's2 | 1.3 |
| Glob Palladus Alzheimer's | 3.1 |
| Glob Palladus Control2 | 1.9 |
| Glob Palladus Control | 1.0 |
| Sub Nigra Depression2 | 1.5 |
| Sub Nigra Depression | 0.8 |
| Sub Nigra PSP2 | 1.2 |
| Sub Nigra Huntington's2 | 10.6 |
| Sub Nigra Huntington's | 15.1 |
| Sub Nigra Parkinson's2 | 12.5 |
| Sub Nigra Alzheimer's2 | 2.0 |
| Sub Nigra Control2 | 9.0 |
| Sub Nigra Control | 8.4 |
| BA17 Depression2 | 6.7 |
| BA17 Depression | 2.0 |
| BA17 PSP2 | 3.6 |
| BA17 PSP | 11.1 |
| BA17 Huntington's2 | 3.4 |
| BA17 Huntington's | 13.7 |
| BA17 Parkinson's2 | 13.5 |
| BA17 Parkinson's | 7.7 |
| BA17 Alzheimer's2 | 1.7 |
| BA17 Control2 | 21.5 |
| BA17 Control | 16.6 |
| BA9 Depression2 | 3.0 |
| BA9 Depression | 2.2 |
| BA9 PSP2 | 2.6 |
| BA9 PSP | 5.1 |
| BA9 Huntington's2 | 2.0 |
| BA9 Huntington's | 16.0 |
| BA9 Parkinson's2 | 33.2 |
| BA9 Parkinson's | 10.2 |
| BA9 Alzheimer's2 | 3.6 |
| BA9 Alzheimer's | 1.1 |
| BA9 Control2 | 100.0 |
| BA9 Control | 8.7 |
| BA7 Depression | 3.6 |
| BA7 PSP2 | 7.8 |
| BA7 PSP | 19.3 |
| BA7 Huntington's2 | 7.6 |
| BA7 Huntington's | 13.1 |
| BA7 Parkinson's2 | 16.8 |
| BA7 Parkinson's | 2.7 |
| BA7 Alzheimer's2 | 1.3 |
| BA7 Control2 | 14.4 |
| BA7 Control | 14.5 |
| BA4 Depression2 | 3.1 |
| BA4 Depression | 6.8 |
| BA4 PSP2 | 15.1 |
| BA4 PSP | 4.6 |
| BA4 Huntington's2 | 0.9 |
| BA4 Huntington's | 21.6 |
| BA4 Parkinson's2 | 40.3 |
| BA4 Parkinson's | 17.1 |
| BA4 Alzheimer's2 | 2.0 |
| BA4 Control2 | 26.4 |
| BA4 Control | 12.5 |

TABLE BBJ
general oncology screening panel_v_2.4

| | Rel. Exp. (%) |
|---|---|
| | Ag1477, |
| | Run |
| Tissue Name | 259733191 |
| Colon cancer 1 | 2.8 |
| Colon NAT 1 | 12.6 |
| Colon cancer 2 | 10.9 |
| Colon NAT 2 | 0.0 |
| Colon cancer 3 | 0.0 |
| Colon NAT 3 | 30.6 |
| Colon malignant cancer 4 | 0.0 |
| Colon NAT 4 | 1.3 |
| Lung cancer 1 | 5.8 |
| Lung NAT 1 | 0.0 |
| Lung cancer 2 | 42.0 |
| Lung NAT 2 | 0.0 |
| Squamous cell carcinoma 3 | 0.0 |
| Lung NAT 3 | 0.0 |
| Metastatic melanoma 1 | 5.3 |
| Melanoma 2 | 2.1 |
| Melanoma 3 | 0.9 |
| Metastatic melanoma 4 | 11.8 |
| Metastatic melanoma 5 | 17.7 |
| Bladder cancer 1 | 1.3 |
| Bladder NAT 1 | 0.0 |
| Bladder cancer 2 | 0.0 |
| Bladder NAT 2 | 0.0 |
| Bladder NAT 3 | 0.0 |
| Bladder NAT 4 | 1.4 |
| Prostate adenocarcinoma 1 | 100.0 |
| Prostate adenocarcinoma 2 | 4.9 |
| Prostate adenocarcinoma 3 | 17.7 |
| Prostate adenocarcinoma 4 | 0.0 |
| Prostate NAT 5 | 8.2 |
| Prostate adenocarcinoma 6 | 7.0 |
| Prostate adenocarcinoma 7 | 2.8 |
| Prostate adenocarcinoma 8 | 5.6 |
| Prostate adenocarcinoma 9 | 48.6 |
| Prostate NAT 10 | 0.0 |
| Kidney cancer 1 | 0.0 |
| Kidney NAT 1 | 6.1 |
| Kidney cancer 2 | 9.7 |
| Kidney NAT 2 | 0.0 |
| Kidney cancer 3 | 1.8 |
| Kidney NAT 3 | 0.0 |
| Kidney cancer 4 | 0.0 |
| Kidney NAT 4 | 0.0 |

AI_comprehensive panel_v1.0 Summary: Ag4105 Highest expression in an sample from OA bone (CT=31.4). Low to moderate levels of expression of this gene are detected in samples derived from osteoarthritic (OA) bone and adjacent bone as well as OA cartilage and OA synovium. Low level expression is also detected in cartilage, bone, and synovial fluid samples from rheumatoid arthritis patients. Low level expression is also detected in samples derived from normal lung samples, COPD lung, emphysema, allergy, Crohn's disease (normal matched control and diseased), and ulcerative colitis (normal matched control and diseased). Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis.

Ardais Panel v.1.0 Summary: Ag1477 Highest expression of this gene is seen in normal lung tissue adjacent to a tumor (CT=31.6). In addition, this gene is expressed at low but significant levels in both lung tumor and normal tissue. The expression in normal adjacent tissue is however, higher compared to the tumor tissue. Therefore, therapeutic modulation of this gene or its protein product may be useful in the treatment of lung cancer

CNS_neurodegeneration_v1.0 Summary: Ag1477/Ag4105 Two experiments with the same probe and primer set produce results that are in excellent agreement. This panel confirms expression of this gene at high levels in the brain, with highest expression detected in the hippocampus of an Alzheiiner's patient (CTs=25-26). In addition, this gene appears to be slightly down-regulated in the temporal cortex of Alzheimer's disease patients. Therefore, up-regulation of this gene or its protein product, or treatment with specific agonists for this receptor may be of use in reversing the dementia, memory loss, and neuronal death associated with this disease.

General_screening_panel_v1.4 Summary: Ag1477/Ag4105 Two experiments with the same probe and primer set produce results that are in excellent agreement. Highest expression of this gene is detected in the fetal brain (CT=25-26). In addition, high to moderate levels of expression of this gene are seen in all regions of the CNS examined, including the hippocampus, thalamus, substantia nigra, amygdala, cerebellum and cerebral cortex. Therefore, therapeutic modulation of the expression or function of this gene may be useful in the treatment of neurological disorders, such as Alzheimer's disease, Parkinson's disease, schizophrenia, multiple sclerosis, stroke and epilepsy.

In addition, this gene is expressed in a cluster of cell line samples derived from lung cancer. This gene is homologous to seizure-related gene 6, a gene clearly involved in lung tumorogenesis. The genetic data from Nishioka et al. point to its genomic region as being involved in lung tumors. While the region itself is often deleted, the expression indicates that the deleted regions might be regulatory region(s) that normally repress the expression of this gene in lung tumor cells. Therefore, targeting this gene with a human monoclonal antibody that results in an inhibition of the activity of this protein, preferably as it relates to its apoptotic/survival activity in tumor cells, specifically lung tumor cells, may have a therapeutic effect on all solid tumor that depend on its activity, preferably on lung tumors.

### REFERENCES

1. Nishioka M. Oncogene Dec. 14, 2000; 19(54):6251-60

2. Shimizu-Nishikawa K. Biochem Biophys Res Commun Nov. 2, 1995;216(1):382-9

Panel 3D Summary: Ag1477 Expression in this panel is consistent with expression in Panel 1.4, with expression detected in samples derived from cerebellum and lung cancer cell lines only.

Panel 4.1D Summary: Ag1477/Ag4105 Two experiments with the same probe and primer set produce results that are in excellent agreement. Highest expression is seen in the kidney, with moderate to low levels of expression seen in resting monocytes, and dendritic cells. The transcript is more highly expressed in resting monocytes and dendritic cells than in treated cells of these types. Thus, the protein encoded by this transcript may be important in monocytic and dendritic cell differentiation and activation. Therefore, regulating the expression of this transcript or the function of the protein it encodes may alter the types and levels of monocytic cells regulated by cytokine and chemokine production and T cell activation. Therapeutics designed with the protein encoded by this transcript could therefore be important for the treatment of asthma, emphysema, inflammatory bowel disease, arthritis and psoriasis.

Panel 5D Summary: Ag1477 Expression of this gene is low/undetectable in all samples on this panel (CTs>35).

Panel CNS_{—}1.1 Summary: Ag1477 This panel confirms the expression of this gene at moderate levels in the brain. See Panels 1.4 and CNS_neurodegeneration_v1.0 for discussion of this gene in the central nervous system.

general oncology screening panel_v_{—}2.4 Summary: Ag1477 Highest expression of this gene is seen in prostate cancer (CT=33). Low but significant levels of expression are also seen in a lung cancer and normal colon. Hence the product of this gene can be used as a marker and therapeutic modulation may lead to treatment of cancer.

BC. CG97012-03: Seizure 6 Precursor Protein-Like Protein.

Expression of gene CG97012-03 was assessed using the primer-probe set Ag6660, described in Table BCA. Results of the RTQ-PCR runs are shown in Tables BCB and BCC.

TABLE BCA

Probe Name Ag6660

| | | | | |
|---|---|---|---|---|
| Primers | Sequences | Length | Start Position | SEQ ID No |

| | | | | |
|---|---|---|---|---|
| Forward | 5′-tatgacatcgtggggagtga-3′ | 20 | 1159 | 777 |
| 0Probe | TET-5′-ctcacctgccagtgggacctcag-3′-TAMRA | 23 | 1183 | 778 |
| Reverse | 5′-gactcctccgttttctcacaa-3′ | 21 | 1227 | 779 |

TABLE BCB
CNS_neurodegeneration_v1.0

| | Rel. Exp. (%) |
|---|---|
| | Ag6660, |
| | Run |
| Tissue Name | 276247136 |
| AD 1 Hippo | 14.8 |
| AD 2 Hippo | 31.0 |
| AD 3 Hippo | 0.0 |
| AD 4 Hippo | 8.2 |
| AD 5 Hippo | 63.3 |
| AD 6 Hippo | 66.0 |
| Control 2 Hippo | 76.3 |
| Control 4 Hippo | 3.9 |
| Control (Path) 3 Hippo | 2.2 |
| AD 1 Temporal Ctx | 2.7 |
| AD 2 Temporal Ctx | 20.0 |
| AD 3 Temporal Ctx | 2.7 |
| AD 4 Temporal Ctx | 11.8 |
| AD 5 Inf Temporal Ctx | 83.5 |
| AD 5 Sup Temporal Ctx | 53.6 |
| AD 6 Inf Temporal Ctx | 29.5 |
| AD 6 Sup Temporal Ctx | 31.0 |
| Control 1 Temporal Ctx | 0.4 |
| Control 2 Temporal Ctx | 47.3 |
| Control 3 Temporal Ctx | 8.2 |
| Control 3 Temporal Ctx | 4.4 |
| Control (Path) 1 Temporal Ctx | 80.1 |
| Control (Path) 2 Temporal Ctx | 32.8 |
| Control (Path) 3 Temporal Ctx | 2.0 |
| Control (Path) 4 Temporal Ctx | 26.4 |
| AD 1 Occipital Ctx | 3.3 |
| AD 2 Occipital Ctx (Missing) | 0.0 |
| AD 3 Occipital Ctx | 2.9 |
| AD 4 Occipital Ctx | 11.7 |
| AD 5 Occipital Ctx | 47.6 |
| AD 6 Occipital Ctx | 11.9 |
| Control 1 Occipital Ctx | 0.8 |
| Control 2 Occipital Ctx | 71.2 |
| Control 3 Occipital Ctx | 8.7 |
| Control 4 Occipital Ctx | 0.0 |
| Control (Path) 1 Occipital Ctx | 100.0 |
| Control (Path) 2 Occipital Ctx | 9.3 |
| Control (Path) 3 Occipital Ctx | 1.4 |
| Control (Path) 4 Occipital Ctx | 10.9 |
| Control 1 Parietal Ctx | 1.8 |
| Control 2 Parietal Ctx | 22.8 |
| Control 3 Parietal Ctx | 13.9 |
| Control (Path) 1 Parietal Ctx | 87.1 |
| Control (Path) 2 Parietal Ctx | 17.3 |
| Control (Path) 3 Parietal Ctx | 1.4 |
| Control (Path) 4 Parietal Ctx | 48.3 |

TABLE BCC
General_screening_panel_v1.6

| | Rel. Exp. (%) |
|---|---|
| | Ag6660, |
| | Run |
| Tissue Name | 277258095 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.0 |
| Lung ca. NCI-N417 | 12.2 |
| Lung ca. LX-1 | 0.0 |
| Lung ca. NCI-H146 | 18.7 |
| Lung ca. SHP-77 | 0.7 |
| Lung ca. A549 | 0.0 |
| Lung ca. NCI-H526 | 4.9 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.2 |
| Renal ca. ACHN | 0.0 |
| Renal ca. U0-31 | 0.0 |
| Renal ca. TK-10 | 0.0 |
| Bladder | 0.1 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.2 |
| Thymus Pool | 0.1 |
| CNS cancer (glio/astro) U87-MG | 0.0 |
| CNS cancer (glio/astro) U-118-MG | 1.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 31.6 |
| Brain (cerebellum) | 99.3 |
| Brain (fetal) | 100.0 |
| Brain (Hippocampus) Pool | 39.2 |
| Cerebral Cortex Pool | 53.6 |
| Brain (Substantia nigra) Pool | 21.0 |
| Brain (Thalamus) Pool | 73.2 |
| Brain (whole) | 67.4 |
| Spinal Cord Pool | 3.6 |
| Adrenal Gland | 1.0 |
| Pituitary gland Pool | 1.6 |
| Salivary Gland | 0.0 |
| Thyroid (female) | 0.0 |
| Pancreatic ca. CAPAN2 | 0.0 |
| Pancreas Pool | 0.0 |

CNS_neurodegeneration_v1.0 Summary: Ag6660 This panel confirms the expression of this gene at low levels in the brains of an independent group of individuals. However, no differential expression of this gene was detected between Alzheimer's diseased postmortem brains and those of non-demented controls in this experiment. See Panel 1.6 for a discussion of this gene in treatment of central nervous system disorders.

General_screening_panel_v1.6 Summary: Ag6660 Highest expression of this gene is detected in fetal brain and cerebellum (CTs=28.8). In addition, moderate levels of expression of this gene is mainly seen in all the regions of central nervous system including amygdala, hippocampus, substantia nigra, thalamus, cerebellum, cerebral cortex, and spinal cord. This gene codes for a variant of Seizure related gene 6 like (SEZ-6/SEZ6L). The expression pattern of this gene is similar to the the one reported in mouse (Shimizu-Nishikawa et al., 1995, Brain Res Mol Brain Res 28:201-10, PMID: 7723619; Biochem Biophys Res Commun 216(1):382-9, PMID: 7488116). Therefore, therapeutic modulation of this gene product may be useful in the treatment of central nervous system disorders such as Alzheimer's disease, Parkinson's disease, epilepsy, multiple sclerosis, schizophrenia and depression.

Moderate levels of expression of this gene is also seen in three of the lung cancer cell lines. Furthermore, genetic and/or epigenetic SEZ6L alterations are involved in the development and/or progression in a subset of lung cancer (Nishioka et al., 2000, Oncogene 19(54):6251-60, PMID: 11175339). Therefore, therapeutic modulation of this gene product through the use of antibodies or small molecule targe may be useful in the treatment of lung cancer.

Panel 4.1D Summary: Ag6660 Expression of this gene is low/undetectable (CTs>35) across all of the samples on this panel (data not shown).

BD. CG99754-01: RIKEN-Like Protein

Expression of gene CG99754-01 was assessed using the primer-probe sets Gpcr07 and Ag07Gpcr, described in Tables BDA and BDB. Results of the RTQ-PCR runs are shown in Tables BDC, BDD, BDE, BDF and BDG.

TABLE BDA
Probe Name Gpcr07

| | | | Start | |
|---|---|---|---|---|
| Primers | Sequences | Length | Position | SEQ ID No |
| Forward | 5′-ctggaggttggcgacaatg-3′ | 19 | 511 | 780 |
| Probe | TET-5′-cctcgtctacatctctcaccgccc-3′-TAMRA | 25 | 531 | 781 |
| Reverse | 5′-ctgctccaggctgttgagg-3′ | 19 | 564 | 782 |

TABLE BDB

Probe Name Ag07Gpcr

| | | | Start | |
|---|---|---|---|---|
| Primers | Sequences | Length | Position | SEQ ID No |
| Forward | 5′-ctggaggttggcgacaatg-3′ | 19 | 511 | 783 |
| Probe | TET-5′-cctcgtctacatctctcaccgcgcc-3′-TAMRA | 25 | 531 | 784 |
| Reverse | 5′-ctgctccaggctgttgagg-3′ | 19 | 564 | 785 |

TABLE BDC
CNS_neurodegeneration_v1.0

| | Rel. Exp. (%) | Rel. Exp. (%) |
|---|---|---|
| | Ag07Gpcr, Run | Gpcr07, Run |
| Tissue Name | 206989718 | 224996509 |
| AD 1 Hippo | 26.2 | 8.5 |
| AD 2 Hippo | 64.2 | 30.4 |
| AD 3 Hippo | 3.5 | 9.4 |
| AD 4 Hippo | 11.3 | 8.2 |
| AD 5 hippo | 59.9 | 78.5 |
| AD 6 Hippo | 81.2 | 40.9 |
| Control 2 Hippo | 53.2 | 39.5 |
| Control 4 Hippo | 11.2 | 6.0 |
| Control (Path) 3 | 3.3 | 4.5 |
| Hippo | | |
| AD 1 Temporal | 17.2 | 11.7 |
| Ctx | | |
| AD 2 Temporal | 34.2 | 30.1 |
| Ctx | | |
| AD 3 Temporal | 1.7 | 7.7 |
| Ctx | | |
| AD 4 Temporal | 28.3 | 18.7 |
| Ctx | | |
| AD 5 Inf | 95.3 | 66.9 |
| Temporal Ctx | | |
| AD 5 | 53.6 | 47.3 |
| SupTemporal | | |
| Ctx | | |
| AD 6 Inf | 55.9 | 36.6 |
| Temporal Ctx | | |
| AD 6 Sup | 78.5 | 37.6 |
| Temporal Ctx | | |
| Control 1 | 5.3 | 7.2 |
| Temporal Ctx | | |
| Control 2 | 61.1 | 56.3 |
| Temporal Ctx | | |
| Control 3 | 28.1 | 18.6 |
| Temporal Ctx | | |
| Control 4 | 15.1 | 9.6 |
| Temporal Ctx | | |
| Control (Path) 1 | 100.0 | 85.9 |
| Temporal Ctx | | |
| Control (Path) 2 | 57.4 | 43.2 |
| Temporal Ctx | | |
| Control (Path) 3 | 2.0 | 5.6 |
| Temporal Ctx | | |
| Control (Path) 4 | 42.9 | 41.2 |
| Temporal Ctx | | |
| AD 1 Occipital | 14.3 | 15.6 |
| Ctx | | |
| AD 2 Occipital | 0.0 | 0.0 |
| Ctx (Missing) | | |
| AD 3 Occipital | 3.6 | 5.0 |
| Ctx | | |
| AD 4 Occipital | 23.8 | 18.8 |
| Ctx | | |
| AD 5 Occipital | 28.5 | 16.8 |
| Ctx | | |
| AD 6 Occipital | 57.0 | 57.0 |
| Ctx | | |
| Control 1 | 0.0 | 4.6 |
| Occipital Ctx | | |
| Control 2 | 46.0 | 68.8 |
| Occipital Ctx | | |
| Control 3 | 21.5 | 18.8 |
| Occipital Ctx | | |
| Control 4 | 3.4 | 6.2 |
| Occipital Ctx | | |
| Control (Path) 1 | 94.0 | 100.0 |
| Occipital Ctx | | |
| Control (Path) 2 | 19.8 | 10.9 |
| Occipital Ctx | | |
| Control (Path) 3 | 1.3 | 3.8 |
| Occipital Ctx | | |
| Control (Path) 4 | 28.9 | 19.1 |
| Occipital Ctx | | |
| Control 1 | 7.5 | 6.6 |
| Parietal Ctx | | |
| Control 2 | 42.3 | 42.3 |
| Parietal Ctx | | |
| Control 3 | 19.1 | 14.0 |
| Parietal Ctx | | |
| Control (Path) 1 | 80.7 | 97.9 |
| Parietal Ctx | | |
| Control (Path) 2 | 30.6 | 27.4 |
| Parietal Ctx | | |
| Control (Path) 3 | 2.0 | 4.0 |
| Parietal Ctx | | |
| Control (Path) 4 | 48.3 | 46.7 |
| Parietal Ctx | | |

TABLE BDD
Panel 1

| | Rel. Exp. (%) |
|---|---|
| | Gpcr07, Run |
| Tissue Name | 109664812 |
| Endothelial cells | 0.0 |
| Endothelial cells (treated) | 0.0 |
| Pancreas | 1.3 |
| Pancreatic ca. CAPAN 2 | 0.0 |
| Adrenal gland | 1.7 |
| Thyroid | 1.3 |
| Salivary gland | 3.0 |
| Pituitary gland | 2.5 |
| Brain (fetal) | 52.5 |
| Brain (whole) | 46.3 |
| Brain (amygdala) | 83.5 |
| Brain (cerebellum) | 19.1 |
| Brain (hippocampus) | 100.0 |
| Brain (substantia nigra) | 43.5 |
| Brain (thalamus) | 59.5 |
| Brain (hypothalamus) | 0.7 |
| Spinal cord | 7.5 |
| glio/astro U87-MG | 0.0 |
| glio/astro U-118-MG | 0.0 |
| astrocytoma SW1783 | 0.0 |
| neuro*; met SK-N-AS | 0.1 |
| astrocytoma SF-539 | 0.8 |
| astrocytoma SNB-75 | 0.0 |
| glioma SNB-19 | 1.5 |
| glioma U251 | 0.2 |
| glioma SF-295 | 0.0 |
| Heart | 5.3 |
| Skeletal muscle | 2.0 |
| Bone marrow | 0.7 |
| Thymus | 0.5 |
| Spleen | 3.4 |
| Lymph node | 0.6 |
| Colon (ascending) | 1.6 |
| Stomach | 1.3 |
| Small intestine | 2.2 |
| Colon ca. SW480 | 0.5 |
| Colon ca.* SW620 | 0.1 |
| (SW480 met) | |
| Colon ca. HT29 | 0.3 |
| Colon ca. HCT-116 | 11.3 |
| Colon ca. CaCo-2 | 0.7 |
| Colon ca. HCT-15 | 0.0 |
| Colon ca. HCC-2998 | 0.4 |
| Gastric ca. * (liver met) NCI-N87 | 0.4 |
| Bladder | 2.7 |
| Trachea | 1.5 |
| Kidney | 2.9 |
| Kidney (fetal) | 51.8 |
| Renal ca. 786-0 | 0.0 |
| Renal ca. A498 | 0.3 |
| Renal ca. RXF 393 | 0.0 |
| Renal ca. ACHN | 0.0 |
| Renal ca. UO-31 | 0.0 |
| Renal ca. TK-10 | 0.0 |
| Liver | 2.1 |
| Liver (fetal) | 3.8 |
| Liver ca. (hepatoblast) HepG2 | 0.0 |
| Lung | 0.0 |
| Lung (fetal) | 0.7 |
| Lung ca. (small cell) LX-1 | 0.0 |
| Lung ca. (small cell) NCI-H69 | 3.7 |
| Lung ca. (s. cell var) SHP-77 | 0.2 |
| Lung ca. (large cell)NCI-H460 | 2.1 |
| Lung ca. (non-sm. cell) A549 | 4.2 |
| Lung ca. (non-s. cell) NCI-H23 | 6.8 |
| Lung ca. (non-s cell) HOP-62 | 0.1 |
| Lung ca. (non-s. cl) NCI-H522 | 3.8 |
| Lung ca. (squam.) SW 900 | 0.3 |
| Lung ca. (squam.) NCI- H596 | 1.6 |
| Mammary gland | 6.4 |
| Breast ca.* (pl. ef) MCF-7 | 1.3 |
| Breast ca.* (pl. ef) MDA-MB-231 | 0.0 |
| Breast ca.* (pl. ef) T47D | 2.6 |
| Breast ca. BT-549 | 0.0 |
| Breast ca. MDA-N | 1.3 |
| Ovary | 5.6 |
| Ovarian ca. OVCAR-3 | 6.2 |
| Ovarian ca. OVCAR-4 | 0.3 |
| Ovarian ca. OVCAR-5 | 1.1 |
| Ovarian ca. OVCAR-8 | 8.0 |
| Ovarian ca. IGROV-1 | 2.1 |
| Ovarian ca. (ascites) SK-OV-3 | 0.1 |
| Uterus | 2.2 |
| Placenta | 2.8 |
| Prostate | 1.5 |
| Prostate ca.* (bone met) PC-3 | 0.0 |
| Testis | 6.3 |
| Melanoma Hs688(A).T | 0.0 |
| Melanoma* (met) Hs688(B).T | 0.1 |
| Melanoma UACC-62 | 9.8 |
| Melanoma M14 | 3.3 |
| Melanoma LOX IMVI | 0.0 |
| Melanoma* (met) SK-MEL-5 | 4.2 |
| Melanoma SK-MEL-28 | 2.9 |

TABLE BDE
Panel 1.2

| | Rel. Exp. (%) | Rel. Exp. (%) |
|---|---|---|
| | Gpcr07, Run | Gpcr07, Run |
| Tissue Name | 124273559 | 126539429 |
| Endothelial cells | 0.0 | 0.1 |
| Heart (Fetal) | 2.8 | 2.8 |
| Pancreas | 0.3 | 0.9 |
| Pancreatic ca. CAPAN 2 | 0.0 | 0.0 |
| Adrenal Gland | 1.7 | 1.5 |
| Thyroid | 0.2 | 0.7 |
| Salivary gland | 1.3 | 1.0 |
| Pituitary gland | 0.4 | 0.6 |
| Brain (fetal) | 17.2 | 28.1 |
| Brain (whole) | 34.9 | 39.2 |
| Brain (amygdala) | 40.6 | 29.7 |
| Brain (cerebellum) | 5.3 | 9.9 |
| Brain (hippocampus) | 45.7 | 51.1 |
| Brain (thalamus) | 10.4 | 15.8 |
| Cerebral Cortex | 100.0 | 100.0 |
| Spinal cord | 3.8 | 3.1 |
| glio/astro U87-MG | 0.0 | 0.0 |
| glio/astro U-118-MG | 0.0 | 0.0 |
| astrocytoma | 0.0 | 0.0 |
| SW1783 | | |
| neuro*; met SK-N-AS | 0.0 | 0.0 |
| astrocytoma SF-539 | 0.2 | 0.2 |
| astrocytoma SNB-75 | 0.0 | 0.0 |
| glioma SNB-19 | 0.4 | 0.3 |
| glioma U251 | 0.0 | 0.1 |
| glioma SF-295 | 0.0 | 0.0 |
| Heart | 2.8 | 1.6 |
| Skeletal Muscle | 0.6 | 0.6 |
| Bone marrow | 0.1 | 0.1 |
| Thymus | 0.1 | 0.1 |
| Spleen | 0.5 | 0.6 |
| Lymph node | 0.4 | 0.3 |
| Colorectal Tissue | 0.4 | 0.3 |
| Stomach | 0.9 | 0.8 |
| Small intestine | 1.7 | 1.5 |
| Colon ca. SW480 | 0.1 | 0.1 |
| Colon ca.* SW620 | 0.1 | 0.1 |
| (SW480 met) | | |
| Colon ca. HT29 | 0.0 | 0.0 |
| Colon ca. HCT-116 | 2.7 | 2.6 |
| Colon ca. CaCo-2 | 0.2 | 0.2 |
| Colon ca. Tissue | 0.4 | 0.3 |
| (ODO3866) | | |
| Colon ca. HCC-2998 | 0.1 | 0.1 |
| Gastric ca.* (liver | 0.2 | 0.2 |
| met) NCI-N87 | | |
| Bladder | 0.9 | 1.0 |
| Trachea | 0.5 | 0.4 |
| Kidney | 0.9 | 1.4 |
| Kidney (fetal) | 3.9 | 3.6 |
| Renal ca. 786-0 | 0.0 | 0.0 |
| Renal ca. A498 | 0.0 | 0.1 |
| Renal ca. RXF 393 | 0.0 | 0.0 |
| Renal ca. ACHN | 0.0 | 0.0 |
| Renal ca. UO-31 | 0.0 | 0.0 |
| Renal ca. TK-10 | 0.0 | 0.0 |
| Liver | 0.7 | 0.6 |
| Liver (fetal) | 1.3 | 1.0 |
| Liver ca. | 0.0 | 0.0 |
| (hepatoblast) HepG2 | | |
| Lung | 0.1 | 0.1 |
| Lung (fetal) | 0.6 | 0.8 |
| Lung ca. (small cell) | 0.0 | 0.1 |
| LX-1 | | |
| Lung ca. (small cell) | 0.7 | 0.5 |
| NCI-H69 | | |
| Lung ca. (s. cell var.) | 0.1 | 0.1 |
| SHP-77 | | |
| Lung ca. (large | 0.8 | 0.5 |
| cell) NCI-H460 | | |
| Lung ca. (non-sm. | 2.0 | 1.0 |
| cell) A549 | | |
| Lung ca. (non-s. cell) | 1.7 | 0.9 |
| NCI-H23 | | |
| Lung ca. (non-s. cell) | 0.0 | 0.0 |
| HOP-62 | | |
| Lung ca. (non-s. cl) | 1.4 | 1.5 |
| NCI-H522 | | |
| Lung ca. (squam.) | 0.1 | 0.1 |
| SW 900 | | |
| Lung ca. (squam.) | 0.4 | 0.5 |
| NCI-H596 | | |
| Mammary gland | 0.8 | 1.3 |
| Breast ca.* (pl. ef) | 0.2 | 0.2 |
| MCF-7 | | |
| Breast ca.* (pl. ef) | 0.0 | 0.0 |
| MDA-MB-231 | | |
| Breast ca.* (pl. ef) | 0.3 | 0.6 |
| T47D | | |
| Breast ca. BT-549 | 0.0 | 0.0 |
| Breast ca. MDA-N | 0.3 | 0.2 |
| Ovary | 2.5 | 1.6 |
| Ovarian ca. | 0.9 | 1.1 |
| OVCAR-3 | | |
| Ovarian ca. | 0.2 | 0.1 |
| OVCAR-4 | | |
| Ovarian ca. | 0.4 | 0.4 |
| OVCAR-5 | | |
| Ovarian ca. | 0.6 | 0.7 |
| OVCAR-8 | | |
| Ovarian ca. IGROV-1 | 0.4 | 0.5 |
| Ovarian ca. (ascites) | 0.0 | 0.1 |
| SK-OV-3 | | |
| Uterus | 1.1 | 1.1 |
| Placenta | 1.2 | 1.1 |
| Prostate | 0.7 | 0.4 |
| Prostate ca.* (bone | 0.0 | 0.0 |
| met) PC-3 | | |
| Testis | 1.4 | 1.7 |
| Melanoma | 0.0 | 0.0 |
| Hs688(A).T | | |
| Melanoma* (met) | 0.0 | 0.0 |
| Hs688(B).T | | |
| Melanoma UACC- | 3.0 | 3.3 |
| 62 | | |
| Melanoma M14 | 0.4 | 0.7 |
| Melanoma LOX | 0.0 | 0.0 |
| IMVI | | |
| Melanoma* (met) | 1.0 | 0.9 |
| SK-MEL-5 | | |

TABLE BDF
Panel 4.1D

| | Rel. Exp. (%) |
|---|---|
| | Ag07Gpcr, Run |
| Tissue Name | 181981925 |
| Secondary Th1 act | 0.0 |
| Secondary Th2 act | 0.0 |
| Secondary Tr1 act | 1.1 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 2.0 |
| Primary Th1 rest | 1.5 |
| Primary Th2 rest | 0.0 |
| Primary Tr1 rest | 0.0 |
| CD45RA CD4 lymphocyte act | 1.1 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 1.0 |
| CD4 lymphocyte none | 0.0 |
| 2ry Th1/Th2/Tr1_anti-CD95 CH11 | 0.0 |
| LAK cells rest | 6.7 |
| LAK cells IL-2 | 0.0 |
| LAK cells IL-2 + IL-12 | 0.0 |
| LAK cells IL-2 + IFN gamma | 0.1 |
| LAK cells IL-2 + IL-18 | 0.0 |
| LAK cells PMA/ionomycin | 1.1 |
| NK Cells IL-2 rest | 0.0 |
| Two Way MLR 3 day | 0.0 |
| Two Way MLR 5 day | 0.0 |
| Two Way MLR 7 day | 2.4 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.2 |
| B lymphocytes CD40L and IL-4 | 0.0 |
| EOL-1 dbcAMP | 0.0 |
| EOL-1 dbcAMP PMA/ionomycin | 1.7 |
| Dendritic cells none | 35.6 |
| Dendritic cells LPS | 30.6 |
| Dendritic cells anti-CD40 | 31.0 |
| Monocytes rest | 0.0 |
| Monocytes LPS | 4.2 |
| Macrophages rest | 28.3 |
| Macrophages LPS | 1.3 |
| HUVEC none | 0.0 |
| HUVEC starved | 3.3 |
| HUVEC IL-1beta | 4.6 |
| HUVEC IFN gamma | 3.1 |
| HUVEC TNF alpha + IFN gamma | 0.0 |
| HUVEC TNF alpha + IL4 | 2.1 |
| HUVEC IL-11 | 1.9 |
| Lung Microvascular EC none | 31.9 |
| Lung Microvascular EC | 7.1 |
| TNFalpha + IL-1beta | |
| Microvascular Dermal EC none | 57.0 |
| Microsvasular Dermal EC | 34.2 |
| TNFalpha + IL-1beta | |
| Bronchial epithelium | 0.0 |
| TNFalpha + IL1beta | |
| Small airway epithelium none | 1.7 |
| Small airway epithelium | 1.7 |
| TNFalpha + IL-1beta | |
| Coronery artery SMC rest | 0.0 |
| Coronery artery SMC | 0.9 |
| TNFalpha + IL-1beta | |
| Astrocytes rest | 1.2 |
| Astrocytes TNFalpha + IL-1beta | 6.4 |
| KU-812 (Basophil) rest | 0.0 |
| KU-812 (Basophil) PMA/ionomycin | 2.0 |
| CCD1106 (Keratinocytes) none | 0.7 |
| CCD1106 (Keratinocytes) | 0.9 |
| TNFalpha + IL-1beta | |
| Liver cirrhosis | 9.4 |
| NCI-H292 none | 1.1 |
| NCI-H292 IL-4 | 0.9 |
| NCI-H292 IL-9 | 0.9 |
| NCI-H292 IL-13 | 1.9 |
| NCI-H292 IFN gamma | 0.9 |
| HPAEC none | 0.0 |
| HPAEC TNF alpha + IL-1 beta | 0.0 |
| Lung fibroblast none | 0.0 |
| Lung fibroblast TNF alpha + IL-1 beta | 1.7 |
| Lung fibroblast IL-4 | 1.1 |
| Lung fibroblast IL-9 | 0.0 |
| Lung fibroblast IL-13 | 2.0 |
| Lung fibroblast IFN gamma | 0.0 |
| Dermal fibroblast CCD1070 rest | 4.8 |
| Dermal fibroblast CCD1070 TNF alpha | 1.0 |
| Dermal fibroblast CCD1070 IL-1beta | 0.0 |
| Dermal fibroblast IFN gamma | 0.0 |
| Dermal fibroblast IL-4 | 1.8 |
| Dermal Fibroblasts rest | 1.8 |
| Neutrophils TNFa + LPS | 2.2 |
| Neutrophils rest | 0.7 |
| Colon | 8.5 |
| Lung | 100.0 |
| Thymus | 11.2 |
| Kidney | 77.4 |

TABLE BDG
Panel CNS_1

| | Rel. Exp. (%) |
|---|---|
| | Gpcr07, Run |
| Tissue Name | 182328460 |
| BA4 Control | 30.8 |
| BA4 Control2 | 70.2 |
| BA4 Alzheimer's2 | 7.1 |
| BA4 Parkinson's | 23.8 |
| BA4 Parkinson's2 | 49.7 |
| BA4 Huntington's | 21.3 |
| BA4 Huntington's2 | 17.0 |
| BA4 PSP | 10.1 |
| BA4 PSP2 | 38.4 |
| BA4 Depression | 34.2 |
| BA4 Depression2 | 10.4 |
| BA7 Control | 33.0 |
| BA7 Control2 | 30.4 |
| BA7 Alzheimer's2 | 9.0 |
| BA7 Parkinson's | 11.7 |
| BA7 Parkinson's2 | 17.2 |
| BA7 Huntington's | 36.3 |
| BA7 Huntington's2 | 21.8 |
| BA7 PSP | 35.8 |
| BA7 PSP2 | 23.7 |
| BA7 Depression | 10.4 |
| BA9 Control | 29.3 |
| BA9 Control2 | 100.0 |
| BA9 Alzheimer's | 7.1 |
| BA9 Alzheimer's2 | 20.3 |
| BA9 Parkinson's | 36.6 |
| BA9 Parkinson's2 | 39.8 |
| BA9 Huntington's | 40.1 |
| BA9 Huntington's2 | 17.3 |
| BA9 PSP | 19.9 |
| BA9 PSP2 | 8.4 |
| BA9 Depression | 15.9 |
| BA9 Depression2 | 12.9 |
| BA17 Control | 40.1 |
| BA17 Control2 | 77.4 |
| BA17 Alzheimer's2 | 11.9 |
| BA17 Parkinson's | 29.3 |
| BA17 Parkinson's2 | 26.4 |
| BA17 Huntington's | 21.8 |
| BA17 Huntington's2 | 18.8 |
| BA17 Depression | 15.5 |
| BA17 Depression2 | 28.5 |
| BA17 PSP | 35.8 |
| BA17 PSP2 | 11.2 |
| Sub Nigra Control | 11.3 |
| Sub Nigra Control2 | 2.4 |
| Sub Nigra Alzheimer's2 | 5.2 |
| Sub Nigra Parkinson's2 | 11.7 |
| Sub Nigra Huntington's | 16.5 |
| Sub Nigra Huntington's2 | 6.2 |
| Sub Nigra PSP2 | 2.8 |
| Sub Nigra Depression | 4.0 |
| Sub Nigra Depression2 | 3.8 |
| Glob Palladus Control | 3.0 |
| Glob Palladus Control2 | 6.3 |
| Glob Palladus Alzheimer's | 5.2 |
| Glob Palladus Alzheimer's2 | 4.6 |
| Glob Palladus Parkinson's | 32.8 |
| Glob Palladus Parkinson's2 | 4.3 |
| Glob Palladus PSP | 2.4 |
| Glob Palladus PSP2 | 5.7 |
| Glob Palladus Depression | 3.7 |
| Temp Pole Control | 21.6 |
| Temp Pole Control2 | 81.2 |
| Temp Pole Alzheimer's | 6.4 |
| Temp Pole Alzheimer's2 | 16.0 |
| Temp Pole Parkinson's | 29.5 |
| Temp Pole Parkinson's2 | 27.0 |
| Temp Pole Huntington's | 33.0 |
| Temp Pole PSP | 57.1 |
| Temp Pole PSP2 | 10.4 |
| Temp Pole Depression2 | 12.1 |
| Cing Gyr Control | 49.0 |
| Cing Gyr Control2 | 76.3 |
| Cing Gyr Alzheimer's | 16.8 |
| Cing Gyr Alzheimer's2 | 17.1 |
| Cing Gyr Parkinson's | 23.5 |
| Cing Gyr Parkinson's2 | 17.7 |
| Cing Gyr Huntington's | 40.6 |
| Cing Gyr Huntington's2 | 12.2 |
| Cing Gyr PSP | 11.8 |
| Cing Gyr PSP2 | 6.3 |
| Cing Gyr Depression | 12.5 |
| Cing Gyr Depression2 | 15.2 |

CNS_neurodegeneration_v1.0 Summary: Ag07Gpcr/Gpcr07 Two runs with the same probe and primer set produce results that are in excellent agreement. This profile confirms the expression of this gene at moderate levels in the brain. This gene appears to be slightly down-regulated in the temporal cortex of Alzheimer's disease patients. Therefore, up-regulation of this gene or its protein product, or treatment with specific agonists for this receptor may be of use in reversing the dementia, memory loss, and neuronal death associated with this disease.

Panel 1 Summary: Gpcr07 Highest expression of this gene is seen in the hippocampus (CT=23), with high levels of detection seen in all regions of the CNS examined. This gene encodes a leucine-rich repeat protein. Leucine rich repeats (LRR) mediate reversible protein-protein interactions and have diverse cellular functions, including cellular adhesion and signaling. Several of these proteins, such as connectin, slit, chaoptin, and Toll have pivotal roles in neuronal development in Drosophila and may play significant but distinct roles in neural development and in the adult nervous system of humans (Ref. 1). In Drosophilia, the LRR region of axon guidance proteins has been shown to be critical for their function (especially in axon repulsion). Since the leucine-rich-repeat protein encoded by this gene shows high expression in the cerebral cortex, it is an excellent candidate neuronal guidance protein for axons, dendrites and/or growth cones in general. Therefore, therapeutic modulation of the levels of this protein, or possible signaling via this protein, may be of utility in enhancing/directing compensatory synaptogenesis and fiber growth in the CNS in response to neuronal death (stroke, head trauma), axon lesion (spinal cord injury), or neurodegeneration (Alzheimer's, Parkinson's, Huntington's, vascular dementia or any neurodegenerative disease).

Moderate to high levels of expression are also seen in cell lines derived from kidney, breast, colon, melanoma, ovarian cancer, lung cancer, and brain cancer. Therefore, therapeutic modulation of the expression or function of this gene product may be effective in the treatment of these cancers.

Among metabolically relevant tissues, this gene expression is seen in skeletal muscle, thyroid, pancreas, adrenal, heart, adult and fetal liver, and pituitary gland. This observation suggests that therapeutic modulation may aid the treatment of metabolic diseases such as obesity and diabetes as well as neuroendocrine disorders. Glycoprotein hormones influence the development and function of the ovary, testis and thyroid by binding to specific high-affinity receptors. The extracellular domains of these receptors are members of the leucine-rich repeat (LRR) protein superfamily and are responsible for the high-affinity binding.

In addition, this gene is expressed at much higher levels in fetal kidney tissue (CT=24) when compared to expression in the adult counterpart (CT=28). Thus, expression of this gene may be used to differentiate between the fetal and adult source of this tissue.

### REFERENCES

1. Jiang X., Dreano M., Buckler D. R., Cheng S., Ythier A., Wu H., Hendrickson W. A., el Tayar N. (1995) Structure 3: 1341-1353.

2. Battye R., Stevens A., Perry R. L., Jacobs J. R. (2001) J. Neurosci. 21: 4290-4298.

3. Itoh A., Miyabayashi T., Ohno M., Sakano S. 1998 Brain Res. Mol. Brain Res. 62: 175-186.

Panel 1.2 Summary: Ag07Gpcr/Gpcr07 Two runs with the same probe and primer set produce results that are in excellent agreement. Highest expression of this gene is seen in the cerebral cortex (CTs=21-22). High levels of expression are seen throughout the CNS, consistent with Panel 1.

Panel 4.1D Summary: Ag07Gpcr Highest expression of this gene is seen in the lung (CT=29.5). Moderate expression is also seen in the kidney, treated and untreated lung and microvascular dermal endothelial cells, treated and untreated dendritic cells, and macrophages. Therefore, therapeutic modulation of this gene may be used for the treatment of autoimmune and inflammatory diseases such as asthma, allergies, inflammatory bowel disease, lupus erythematosus, psoriasis, rheumatoid arthritis, and osteoarthritis.

Panel CNS_{—}1 Summary: Gpcr07 This panel confirms the expression of this gene at high levels in the brain. See Panels 1 and CNS_neurodegeneration_v1.0 for discussion of this gene in the central nervous system.

BE. CG99777-02: CD30 Ligand-Like Protein.

Expression of gene CG99777-02 was assessed using the primer-probe sets Ag6623, Ag6747 and Ag6919, described in Tables BEA, BEB and BEC. Results of the RTQ-PCR runs are shown in Tables BED, BEE and BEF. Note that CG99777-02 represents a full-length physical clone.

TABLE BEA

Probe Name Ag6623

| | | | Start | |
|---|---|---|---|---|
| Primers | Sequences | Length | Position | SEQ ID No |
| Forward | 5′-agaaagcgcctctctaccatac-3′ | 22 | 745 | 786 |
| Probe | TET-5′-tatttcatccctccaaacacttgggc-3′-TAMRA | 26 | 769 | 787 |
| Reverse | 5′-gaggagaatccttcttggtctaaa-3′ | 24 | 806 | 788 |

TABLE BEB

Probe Name Ag6747

| | | | Start | |
|---|---|---|---|---|
| Primers | Sequences | Length | Position | SEQ ID No |
| Forward | 5′-aaaggagtggcaaagcatct-3′ | 20 | 320 | 789 |
| Probe | TET-5′-catggagaatgccatctttgttccaa-3′-TAMRA | 26 | 358 | 790 |
| Reverse | 5′-ccagattcccatcctgatatc-3′ | 21 | 390 | 791 |

TABLE BEC

Probe Name Ag6919

| | | | Start | SEQ ID |
|---|---|---|---|---|
| Primers | Sequences | Length | Position | No |
| Forward | 5′-cctcaaggagtggcaaagc-3′ | 20 | 316 | 792 |
| Probe | TET-5′-caaaaccaagttgtcttggaacaaagatgqcattctcc-3′-TAMRA | 38 | 343 | 793 |
| Reverse | 5′-ccagattcccatcctgatatctga-3′ | 24 | 387 | 794 |

TABLE BED
AI_comprehensive panel_v1.0

| | Rel. Exp. (%) |
|---|---|
| | Ag6919, Run |
| Tissue Name | 308380234 |
| 110967 COPD-F | 6.9 |
| 110980 COPD-F | 9.0 |
| 110968 COPD-M | 7.0 |
| 110977 COPD-M | 13.0 |
| 110989 Emphysema-F | 7.8 |
| 110992 Emphysema-F | 4.5 |
| 110993 Emphysema-F | 3.4 |
| 110994 Emphysema-F | 2.6 |
| 110995 Emphysema-F | 7.1 |
| 110996 Emphysema-F | 1.3 |
| 110997 Asthma-M | 3.3 |
| 111001 Asthma-F | 11.3 |
| 111002 Asthma-F | 9.9 |
| 111003 Atopic Asthma-F | 8.8 |
| 111004 Atopic Asthma-F | 8.0 |
| 111005 Atopic Asthma-F | 3.8 |
| 111006 Atopic Asthma-F | 0.8 |
| 111417 Allergy-M | 4.4 |
| 112347 Allergy-M | 0.0 |
| 112349 Normal Lung-F | 0.0 |
| 112357 Normal Lung-F | 6.7 |
| 112354 Normal Lung-M | 3.5 |
| 112374 Crohns-F | 4.7 |
| 112389 Match Control Crohns-F | 8.2 |
| 112375 Crohns-F | 3.3 |
| 112732 Match Control Crohns-F | 50.0 |
| 112725 Crohns-M | 1.9 |
| 112387 Match Control Crohns-M | 3.7 |
| 112378 Crohns-M | 0.2 |
| 112390 Match Control Crohns-M | 6.7 |
| 112726 Crohns-M | 12.6 |
| 112731 Match Control Crohns-M | 5.1 |
| 112380 Ulcer Col-F | 2.3 |
| 112734 Match Control Ulcer Col-F | 100.0 |
| 112384 Ulcer Col-F | 6.8 |
| 112737 Match Control Ulcer Col-F | 4.0 |
| 112386 Ulcer Col-F | 4.0 |
| 112738 Match Control Ulcer Col-F | 9.2 |
| 112381 Ulcer Col-M | 0.2 |
| 112735 Match Control Ulcer Col-M | 0.8 |
| 112382 Ulcer Col-M | 7.3 |
| 112394 Match Control Ulcer Col-M | 1.4 |
| 112383 Ulcer Col-M | 4.8 |
| 112736 Match Control Ulcer Col-M | 3.9 |
| 112423 Psoriasis-F | 5.0 |
| 112427 Match Control Psoriasis-F | 10.0 |
| 112418 Psoriasis-M | 3.5 |
| 112723 Match Control Psoriasis-M | 2.7 |
| 112419 Psoriasis-M | 8.8 |
| 112424 Match Control Psoriasis-M | 3.3 |
| 112420 Psoriasis-M | 17.9 |
| 112425 Match Control Psoriasis-M | 9.6 |
| 104689 (MF) OA Bone-Backus | 50.3 |
| 104690 (MF) Adj “Normal” Bone-Backus | 27.9 |
| 104691 (MF) OA Synovium-Backus | 47.3 |
| 104692 (BA) OA Cartilage-Backus | 0.0 |
| 104694 (BA) OA Bone-Backus | 32.8 |
| 104695 (BA) Adj “Normal” Bone-Backus | 27.2 |
| 104696 (BA) OA Synovium-Backus | 82.4 |
| 104700 (SS) OA Bone-Backus | 30.6 |
| 104701 (SS) Adj “Normal” Bone-Backus | 40.9 |
| 104702 (SS) OA Synovium-Backus | 66.4 |
| 117093 OA Cartilage Rep7 | 6.3 |
| 112672 OA Bone5 | 11.0 |
| 112673 OA Synovium5 | 5.8 |
| 112674 OA Synovial Fluid cells5 | 5.1 |
| 117100 OA Cartilage Rep14 | 2.8 |
| 112756 OA Bone9 | 3.2 |
| 112757 OA Synovium9 | 2.3 |
| 112758 OA Synovial Fluid Cells9 | 6.8 |
| 117125 RA Cartilage Rep2 | 7.1 |
| 113492 Bone2 RA | 16.5 |
| 113493 Synovium2 RA | 5.3 |
| 113494 Syn Fluid Cells RA | 6.0 |
| 113499 Cartilage4 RA | 9.9 |
| 113500 Bone4 RA | 5.5 |
| 113501 Synovium4 RA | 5.8 |
| 113502 Syn Fluid Cells4 RA | 6.2 |
| 113495 Cartilage3 RA | 7.8 |
| 113496 Bone3 RA | 9.5 |
| 113497 Synovium3 RA | 7.5 |
| 113498 Syn Fluid Cells3 RA | 11.3 |
| 117106 Normal Cartilage Rep20 | 0.8 |
| 113663 Bone3 Normal | 0.0 |
| 113664 Synovium3 Normal | 0.0 |
| 113665 Syn Fluid Cells3 Normal | 0.1 |
| 117107 Normal Cartilage Rep22 | 1.3 |
| 113667 Bone4 Normal | 1.8 |
| 113668 Synovium4 Normal | 3.0 |
| 113669 Syn Fluid Cells4 Normal | 4.7 |

TABLE BEE
General_screening_panel_v1.6

| | Rel. Exp. (%) |
|---|---|
| | Ag6919, Run |
| Tissue Name | 278388682 |
| Adipose | 23.7 |
| Melanoma* Hs688(A).T | 0.0 |
| Melanoma* Hs688(B).T | 0.0 |
| Melanoma* M14 | 0.0 |
| Melanoma* LOXIMVI | 1.4 |
| Melanoma* SK-MEL-5 | 0.0 |
| Squamous cell carcinoma SCC-4 | 0.0 |
| Testis Pool | 2.6 |
| Prostate ca.* (bone met) PC-3 | 0.0 |
| Prostate Pool | 5.2 |
| Placenta | 5.7 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 9.9 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 7.3 |
| Trachea | 12.7 |
| Lung | 0.0 |
| Fetal Lung | 15.5 |
| Lung ca. NCI-N417 | 0.0 |
| Lung ca. LX-1 | 0.0 |
| Lung ca. NCI-H146 | 0.0 |
| Lung ca. SHP-77 | 4.7 |
| Lung ca. A549 | 0.0 |
| Lung ca. NCI-H526 | 100.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 5.6 |
| Liver ca. HepG2 | 0.0 |
| Kidney Pool | 13.9 |
| Fetal Kidney | 3.4 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 16.4 |
| Gastric ca. (liver met.) NCI-N87 | 0.8 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 2.4 |
| Colon cancer tissue | 15.5 |
| Colon ca. SW1116 | 0.0 |
| Colon ca. Colo-205 | 0.0 |
| Colon ca. SW-48 | 0.0 |
| Colon Pool | 15.0 |
| Small Intestine Pool | 4.3 |
| Stomach Pool | 5.6 |
| Bone Marrow Pool | 2.6 |
| Fetal Heart | 1.4 |
| Heart Pool | 2.8 |
| Lymph Node Pool | 8.7 |
| Fetal Skeletal Muscle | 0.0 |
| Skeletal Muscle Pool | 1.7 |
| Spleen Pool | 11.5 |
| Thymus Pool | 46.3 |
| CNS cancer (glio/astro) U87-MG | 2.2 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 2.4 |
| Brain (cerebellum) | 1.8 |
| Brain (fetal) | 0.0 |
| Brain (Hippocampus) Pool | 1.7 |
| Cerebral Cortex Pool | 0.0 |
| Brain (Substantia nigra) Pool | 0.0 |
| Brain (Thalamus) Pool | 1.0 |
| Brain (whole) | 1.7 |
| Spinal Cord Pool | 4.3 |
| Adrenal Gland | 5.2 |
| Pituitary gland Pool | 0.0 |
| Salivary Gland | 1.8 |
| Thyroid (female) | 0.8 |
| Pancreatic ca. CAPAN2 | 0.0 |
| Pancreas Pool | 1.7 |

TABLE BEF
Panel 4.1D

| | Rel.Exp. (%) | Rel.Exp. (%) |
|---|---|---|
| | Ag6747, Run | Ag6919, Run |
| Tissue Name | 277641366 | 306067437 |
| Secondary Th1 act | 8.1 | 8.1 |
| Secondary Th2 act | 12.3 | 17.6 |
| Secondary Tr1 act | 3.3 | 5.1 |
| Secondary Th1 rest | 3.8 | 3.4 |
| Secondary Th2 rest | 4.0 | 3.9 |
| Secondary Tr1 rest | 6.0 | 7.1 |
| Primary Th1 act | 47.3 | 9.7 |
| Primary Th2 act | 100.0 | 59.5 |
| Primary Tr1 act | 82.4 | 100.0 |
| Primary Th1 rest | 1.3 | 1.2 |
| Primary Th2 rest | 1.6 | 1.7 |
| Primary Tr1 rest | 0.5 | 0.5 |
| CD45RA CD4 lymphocyte act | 6.0 | 6.8 |
| CD45RO CD4 lymphocyte act | 12.4 | 19.1 |
| CD8 lymphocyte act | 1.3 | 1.1 |
| Secondary CD8 lymphocyte rest | 1.9 | 1.2 |
| Secondary CD8 lymphocyte act | 1.0 | 0.4 |
| CD4 lymphocyte none | 3.1 | 2.4 |
| 2ry Th1/Th2/Tr1_anti-CD95 CH11 | 2.3 | 3.0 |
| LAK cells rest | 3.6 | 3.3 |
| LAK cells IL-2 | 1.2 | 1.2 |
| LAK cells IL-2 + IL-12 | 0.3 | 0.3 |
| LAK cells IL-2 + IFN gamma | 1.8 | 2.1 |
| LAK cells IL-2 + IL-18 | 0.8 | 0.9 |
| LAK cells PMA/ionomycin | 12.9 | 18.7 |
| NK Cells IL-2 rest | 6.4 | 5.4 |
| Two Way MLR 3 day | 2.1 | 1.6 |
| Two Way MLR 5 day | 0.4 | 0.6 |
| Two Way MLR 7 day | 0.6 | 0.5 |
| PBMC rest | 1.1 | 2.1 |
| PBMC PWM | 0.7 | 0.3 |
| PBMC PHA-L | 1.0 | 1.2 |
| Ramos (B cell) none | 3.6 | 4.7 |
| Ramos (B cell) ionomycin | 15.4 | 15.1 |
| B lymphocytes PWM | 2.6 | 3.0 |
| B lymphocytes CD40L and IL-4 | 4.8 | 4.1 |
| EOL-1 dbcAMP | 0.0 | 0.0 |
| EOL-1 dbcAMP PMA/ionomycin | 0.1 | 0.0 |
| Dendritic cells none | 1.6 | 2.7 |
| Dendritic cells LPS | 1.3 | 1.2 |
| Dendritic cells anti-CD40 | 1.1 | 1.4 |
| Monocytes rest | 2.2 | 3.3 |
| Monocytes LPS | 9.4 | 9.4 |
| Macrophages rest | 1.1 | 0.3 |
| Macrophages LPS | 0.5 | 0.5 |
| HUVEC none | 0.0 | 0.0 |
| HUVEC starved | 0.0 | 0.0 |
| HUVEC IL-1beta | 0.1 | 0.0 |
| HUVEC IFN gamma | 0.0 | 0.0 |
| HUVEC TNF alpha + IFN gamma | 0.0 | 0.0 |
| HUVEC TNF alpha + IL4 | 0.0 | 0.0 |
| HUVEC IL-11 | 1.4 | 0.0 |
| Lung Microvascular EC none | 0.0 | 0.0 |
| Lung Microvascular EC | 0.0 | 0.0 |
| TNFalpha + IL-1beta | | |
| Microvascular Dermal EC none | 0.0 | 0.0 |
| Microsvasular Dermal EC | 0.0 | 0.0 |
| TNFalpha + IL-1beta | | |
| Bronchial epithelium | 0.0 | 0.0 |
| TNFalpha + IL1beta | | |
| Small airway epithelium none | 0.0 | 0.0 |
| Small airway epithelium | 0.0 | 0.0 |
| TNFalpha + IL-1beta | | |
| Coronery artery SMC rest | 0.0 | 0.0 |
| Coronery artery SMC | 0.0 | 0.0 |
| TNFalpha + IL-1beta | | |
| Astrocytes rest | 0.0 | 0.0 |
| Astrocytes TNFalpha + IL-1beta | 0.0 | 0.0 |
| KU-812 (Basophil) rest | 0.0 | 0.0 |
| KU-812 (Basophil) PMA/ionomycin | 0.0 | 0.0 |
| CCD1106 (Keratinocytes) none | 0.0 | 0.0 |
| CCD1106 (Keratinocytes) | 0.0 | 0.0 |
| TNFalpha + IL-1beta | | |
| Liver cirrhosis | 0.2 | 0.3 |
| NCI-H292 none | 0.0 | 0.0 |
| NCI-H292 IL-4 | 0.0 | 0.0 |
| NCI-H292 IL-9 | 0.0 | 0.0 |
| NCI-H292 IL-13 | 0.0 | 0.0 |
| NCI-H292 IFN gamma | 0.0 | 0.0 |
| HPAEC none | 0.0 | 0.0 |
| HPAEC TNF alpha + IL-1 beta | 0.2 | 0.0 |
| Lung fibroblast none | 0.0 | 0.0 |
| Lung fibroblast TNFalpha + | 0.0 | 0.0 |
| IL-1 beta | | |
| Lung fibroblast IL-4 | 0.0 | 0.0 |
| Lung fibroblast IL-9 | 0.0 | 0.0 |
| Lung fibroblast IL-13 | 0.0 | 0.0 |
| Lung fibroblast IFN gamma | 0.0 | 0.0 |
| Dermal fibroblast CCD1070 rest | 0.0 | 0.0 |
| Dermal fibroblast CCD1070 TNF alpha | 6.3 | 4.6 |
| Dermal fibroblast CCD1070 IL-1 beta | 0.0 | 0.0 |
| Dermal fibroblast IFN gamma | 0.0 | 0.0 |
| Dermal fibroblast IL-4 | 0.0 | 0.0 |
| Dermal Fibroblasts rest | 0.0 | 0.0 |
| Neutrophils TNFa + LPS | 3.7 | 4.9 |
| Neutrophils rest | 2.1 | 3.3 |
| Colon | 0.1 | 0.0 |
| Lung | 0.1 | 0.0 |
| Thymus | 1.5 | 3.0 |
| Kidney | 0.1 | 0.0 |

AI_comprehensive panel_v1.0 Summary: Ag6919 Highest expression of this gene is seen in a normal tissue adjacent to ulcerative colitis (CT=29.5). This gene is widely expressed in this panel, with moderate levels of expression in a cluster of OA samples. Thus, expression of this gene could be used to differentiate between the OA samples and other samples on this panel, and as a marker of OA. Furthermore, therapeutic modulation of the expression or function of this gene may be useful in the treatment of OA.

General_screening_panel_v1.6 Summary: Ag6919 Expression of this gene is restricted to a sample derived from a lung cancer cell line and from the thymus(CTs=33-34). Thus, expression of this gene could be used to differentiate between these samples and other samples on this panel and as a marker to detect the presence of lung cancer. Furthermore, therapeutic modulation of the expression or function of this gene may be effective in the treatment of lung cancer.

Panel 4.1D Summary: Ag6747/Ag6919 Expression is highest in acutely activated T cells (CTs=25-30). This gene is expressed at higher levels during primary activation of Th2 and Tr1 cells. Thus, this gene may be important for early Th2 cell differentiation and Th2 related immune disorders such as asthma. This gene encodes a protein with homology to CD30-L, a member of the tumor necrosis factor receptor superfamily expressed on the surface of activated T cells. Thus based on this expression profile, therapeutics designed with the protein encoded by this transcript could be important in the regulation of T cell function. In addition, therapeutic regulation of the transcript or the protein encoded by the transcript could be important in immune modulation and in the treatment of T cell-mediated diseases such as asthma, arthritis, psoriasis, inflammatory bowel disease, and lupus.

Ag6623 Expression of this gene is low/undetectable in all samples on this panel (CTs>35).

### Example D

### Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTools™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al, Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV1b SNP Data (CG108440-02) Three polymorphic variants of NOV1b have been identified and are shown in Table 41A.

TABLE 41A
NOV1b SNP Data

| | Nucleotides | | | Amino Acids | | |
|---|---|---|---|---|---|---|
| Variant | Position | Initial | Modified | Position | Initial | Modified |
| 13380440 | 1921 | C | T | 639 | Ser | Phe |
| 13378327 | 4730 | A | G | 1575 | Glu | Glu |
| 13378325 | 6395 | T | C | 2130 | Tyr | Tyr |

NOV4a SNP Data (CG1344340-01)

One polymorphic variant of NOV4a has been identified and is shown in Table 41B.

TABLE 41B
NOV4a SNP Data

| | Nucleotides | | | Amino Acids | | |
|---|---|---|---|---|---|---|
| Variant | Position | Initial | Modified | Position | Initial | Modified |
| 13380480 | 652 | G | A | 144 | Trp | End |

NOV8b SNP Data (CG137793-02)

Twenty polymorphic variants of NOV8b have been identified and are shown in Table 41C.

TABLE 41C
NOV8b SNP Data

| | Nucleotides | | | Amino Acids | | |
|---|---|---|---|---|---|---|
| Variant | Position | Initial | Modified | Position | Initial | Modified |
| 13380443 | 52 | T | C | 9 | Thr | Thr |
| 13380444 | 65 | G | A | 14 | Ala | Thr |
| 13380445 | 80 | G | A | 19 | Ala | Thr |
| 13380446 | 146 | A | G | 41 | Ile | Val |
| 13380447 | 168 | C | T | 48 | Thr | Ile |
| 13380448 | 190 | T | C | 55 | Asn | Asn |
| 13380449 | 211 | C | T | 62 | Thr | Thr |
| 13380450 | 234 | T | C | 70 | Leu | Pro |
| 13380451 | 241 | A | G | 72 | Glu | Glu |
| 13380452 | 242 | G | T | 73 | Glu | End |
| 13380459 | 355 | T | C | 110 | Gly | Gly |
| 13380460 | 486 | C | T | 154 | Thr | Ile |
| 13380461 | 497 | G | A | 158 | Ala | Thr |
| 13380462 | 579 | T | C | 185 | Ile | Thr |
| 13380463 | 586 | T | C | 187 | Thr | Thr |
| 13380464 | 597 | T | C | 191 | Val | Ala |
| 13380465 | 609 | T | C | 195 | Leu | Ser |
| 13380466 | 649 | C | A | 208 | Asn | Lys |
| 13380467 | 665 | A | G | 214 | Asn | Asp |
| 13380468 | 694 | A | G | 0 | | |

NOV16a SNP Data (CG138751-01)

Two polymorphic variants of NOV16a have been identified and are shown in Table 41D.

TABLE 41D
SNP Data

| | Nucleotides | | | Amino Acids | | |
|---|---|---|---|---|---|---|
| Variant | Position | Initial | Modified | Position | Initial | Modified |
| 13380684 | 70 | T | C | 4 | Ser | Pro |
| 13380685 | 411 | C | T | 117 | Tyr | Tyr |

NOV17b SNP Data (CG139062-02)

Five polymorphic variants of NOV17b have been identified and are shown in Table 41E.

TABLE 41E
NOV17b SNP Data

| | Nucleotides | | | Amino Acids | | |
|---|---|---|---|---|---|---|
| Variant | Position | Initial | Modified | Position | Initial | Modified |
| 13380469 | 680 | G | A | 89 | Gly | Gly |
| 13380470 | 801 | A | G | 130 | Arg | Gly |
| 13380471 | 1178 | T | C | 255 | Tyr | Tyr |
| 13380475 | 2684 | C | T | 757 | Tyr | Tyr |
| 13380476 | 3945 | G | T | 0 | | |

NOV20a SNP Data (CG140305-01)

Two polymorphic variants of NOV20a have been identified and are shown in Table 41F.

TABLE 41F
NOV20a SNP Data

| | Nucleotides | | | Amino Acids | | |
|---|---|---|---|---|---|---|
| Variant | Position | Initial | Modified | Position | Initial | Modified |
| 13380503 | 218 | G | T | 57 | Gly | Val |
| 13380501 | 404 | T | C | 119 | Val | Ala |

NOV22a SNP Data (CG140843-01)

One polymorphic variant of NOV22a has been identified and is shown in Table 41G.

TABLE 41G
NOV22a SNP Data

| | Nucleotides | | | Amino Acids | | |
|---|---|---|---|---|---|---|
| Variant | Position | Initial | Modified | Position | Initial | Modified |
| 13380504 | 217 | A | G | 57 | Pro | Pro |

NOV23a SNP Data (CG141540-01)

Six plymorphic variants of NOV23a have been identified and are shown in Table 41H.

TABLE 41H
NOV23a SNP Data

| | Nucleotides | | | Amino Acids | | |
|---|---|---|---|---|---|---|
| Variant | Position | Initial | Modified | Position | Initial | Modified |
| 13380496 | 132 | A | G | 22 | Thr | Thr |
| 13377809 | 184 | A | G | 40 | Arg | Gly |
| 13377810 | 227 | C | T | 54 | Pro | Leu |
| 13378253 | 258 | C | A | 64 | Arg | Arg |
| 13377812 | 823 | T | C | 253 | Trp | Arg |
| 13378251 | 1044 | G | A | 326 | Thr | Thr |

NOV24a SNP Data (CG14580-01)

Two polymorphic variants of NOV24a have been identified and are shown in Table 41I.

TABLE 41I
NOV24a SNP Data

| | Nucleotides | | | Amino Acids | | |
|---|---|---|---|---|---|---|
| Variant | Position | Initial | Modified | Position | Initial | Modified |
| 13380498 | 197 | G | A | 46 | Gly | Glu |
| 13380497 | 1693 | G | A | 545 | Ala | Thr |

NOV26a SNP Data (CG142003-01)

One polymorphic variant of NOV26a has been identified and is shown in Table 41J.

TABLE 41J
NOV26a SNP Data

| | Nucleotides | | | Amino Acids | | |
|---|---|---|---|---|---|---|
| Variant | Position | Initial | Modified | Position | Initial | Modified |
| 13380544 | 375 | G | A | 125 | Val | Met |

NOV29c SNP Data (CG171681-02)

Two polymorphic variants of NOV29c have been identified and are shown in Table 41K.

TABLE 41K
NOV29c SNP Data

| | Nucleotides | | | Amino Acids | | |
|---|---|---|---|---|---|---|
| Variant | Position | Initial | Modified | Position | Initial | Modified |
| 13380521 | 361 | A | G | 96 | Lys | Arg |
| 13380522 | 1646 | G | C | 0 | | |

NOV32a SNP Data (CG52423-01)

Twenty polymorphic variants of NOV32a have been identified and are shown in Table 41L.

TABLE 41L
NOV32a SNP Data

| | Nucleotides | | | Amino Acids | | |
|---|---|---|---|---|---|---|
| Variant | Position | Initial | Modified | Position | Initial | Modified |
| 13380557 | 26 | C | T | 0 | | |
| 13380556 | 54 | A | G | 1 | Met | Val |
| 13380555 | 60 | G | A | 3 | Ala | Thr |
| 13380545 | 357 | A | G | 102 | Asn | Asp |
| 13380546 | 562 | A | G | 170 | Asn | Ser |
| 13380547 | 739 | A | G | 229 | Glu | Gly |
| 13380548 | 760 | A | G | 236 | Gln | Arg |
| 13380549 | 774 | C | T | 241 | Gln | End |
| 13380550 | 796 | A | G | 248 | Asp | Gly |
| 13380551 | 843 | A | G | 264 | Ile | Val |
| 13380552 | 868 | T | C | 272 | Leu | Pro |
| 13380553 | 892 | T | C | 280 | Leu | Pro |
| 13380554 | 893 | G | A | 280 | Leu | Leu |
| 13380542 | 1066 | A | G | 338 | Gln | Arg |
| 13380558 | 1073 | G | A | 340 | Ala | Ala |
| 13374369 | 1133 | C | G | 360 | Ser | Ser |
| 13380559 | 1151 | G | A | 366 | Ala | Ala |
| 13380560 | 1288 | A | G | 412 | Lys | Arg |
| 13380515 | 1303 | T | C | 417 | Met | Thr |
| 13374368 | 1357 | C | T | 435 | Ala | Val |

NOV34b SNP Data (CG55698-02)

Four polymorphic variants of NOV34b have been identified and are shown in Table 41M

TABLE 41M
NOV34b SNP Data

| | Nucleotides | | | Amino Acids | | |
|---|---|---|---|---|---|---|
| Variant | Position | Initial | Modified | Position | Initial | Modified |
| 13380563 | 170 | G | A | 52 | Val | Met |
| 13380564 | 210 | A | G | 65 | Asp | Gly |
| 13380565 | 218 | C | T | 68 | Arg | Cys |
| 13380566 | 249 | C | T | 0 | | |

NOV35c SNP Data (CG55832-02)

Twelve polymorphic variants of NOV35c have been identified and are shown in Table 41N.

TABLE 41N
NOV35c SNP Data

| | Nucleotides | | | Amino Acids | | |
|---|---|---|---|---|---|---|
| Variant | Position | Initial | Modified | Position | Initial | Modified |
| 13380671 | 330 | A | G | 92 | Val | Val |
| 13380672 | 638 | A | G | 195 | His | Arg |
| 13380673 | 750 | G | A | 232 | Val | Val |
| 13380674 | 1260 | T | C | 402 | Cys | Cys |
| 13378289 | 2090 | G | A | 679 | Arg | Gln |
| 13380675 | 2100 | G | A | 682 | Glu | Glu |
| 13380676 | 3342 | A | G | 1096 | Thr | Thr |
| 13380677 | 3813 | A | G | 1253 | Arg | Arg |
| 13380678 | 4118 | C | T | 1355 | Thr | Met |
| 13378291 | 4162 | C | G | 1370 | Gln | Glu |
| 13380705 | 4397 | T | C | 1448 | Val | Ala |
| 13380704 | 5269 | A | G | 0 | | |

NOV37a SNP Data (CG88634-01)

Two polymorphic variants of NOV37a have been identified and are shown in Table 41O.

TABLE 41O
NOV37a SNP Data

| | Nucleotides | | | Amino Acids | | |
|---|---|---|---|---|---|---|
| Variant | Position | Initial | Modified | Position | Initial | Modified |
| 13380706 | 165 | T | C | 22 | Leu | Pro |
| 13380707 | 272 | A | C | 58 | Lys | Gln |

NOV38a SNP Data (CG97012-01)

Two polymorphic variants of NOV38a have been identified and are shown in Table 41P.

TABLE 41P
NOV38a SNP Data

| | Nucleotides | | | Amino Acids | | |
|---|---|---|---|---|---|---|
| Variant | Position | Initial | Modified | Position | Initial | Modified |
| 13380698 | 828 | G | A | 276 | Gln | Gln |
| 13380699 | 1271 | A | G | 424 | Glu | Gly |

NOV39a SNP Data (CG99754-01)

Six polymorphic variants of NOV39a have been identified and are shown in Table 41Q.

TABLE 41Q
NOV39a SNP Data

| | Nucleotides | | | Amino Acids | | |
|---|---|---|---|---|---|---|
| Variant | Position | Initial | Modified | Position | Initial | Modified |
| 13374727 | 314 | T | C | 100 | Ile | Thr |
| 13374728 | 362 | G | A | 116 | Arg | Gln |
| 13380709 | 545 | C | T | 177 | Ser | Phe |
| 13380710 | 711 | G | C | 232 | Leu | Leu |
| 13380711 | 1355 | C | T | 447 | Pro | Leu |
| 13380712 | 1886 | C | T | 0 | | |

NOV40b SNP Data (CG99777-02)

Three polymorphic variants of NOV40b have been identified and are shown in Table 41R.

TABLE 41R
NOV40b SNP Data

| | Nucleotides | | | Amino Acids | | |
|---|---|---|---|---|---|---|
| Variant | Position | Initial | Modified | Position | Initial | Modified |
| 13378112 | 466 | A | G | 126 | Gln | Gln |
| 13378113 | 498 | A | G | 137 | Glu | Gly |
| 13378114 | 641 | G | A | 185 | Asp | Asn |

NOV30b SAGE Expression Data

Construction of the mammalian expression vector pCEP4/Sec. The oligonucleotide primers, pSec-V5-His Forward (CTCGTC CTCGAG GGT AAG CCT ATC CCT AAC; SEQ ID NO:795) and the pSec-V5-His Reverse (CTCGTCGGGCCCCTGATCAGCGGGTTTAAAC; SEQ ID NO:796), were designed to amplify a fragment from the pcDNA3.1-V5His (Invitrogen, Carlsbad, Calif.) expression vector. The PCR product was digested with XhoI and ApaI and ligated into the XhoI/ApaI digestedpSecTag2 B vector (Invitrogen, Carlsbad Calif.). The correct structure of the resulting vector, pSecV5His, was verified by DNA sequence analysis. The vector pSecV5His was digested with PmeI and NheI, and the PmeI-NheI fragment was ligated into the BamHI/Klenow and NheI treated vector pCEP4 (Invitrogen, Carlsbad, Calif.). The resulting vector was named as pCEP4/Sec.

Expression of CG51117-05 in human embryonic kidney 293 cells. A 1.6 kb BamHI-XhoI fragment containing the CG5117-05 sequence was subcloned into BamHI-XhoI digested pCEP4/Sec to generate plasmid 163. The resulting plasmid 163 was transfected into 293 cells using the LipofectaininePlus reagent following the manufacturer's instructions (Gibco/BRL). The cell pellet and supernatant were harvested 7211 post transfection and examined for CG51117-05 expression by Western blot (reducing conditions) using an anti-V5 antibody. FIG. 1 shows that CG51117-05 is expressed as an approximately 66 kDa protein, secreted by 293 cells.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO:2n, wherein n is an integer between 1 and 127.

2. An isolated polypeptide comprising an amino acid sequence selected from the group consisting of SEQ ID NO:2n, wherein n is an integer between 1 and 127.

3. An isolated polypeptide comprising an amino acid sequence which is at least 95% identical to an amino acid sequence selected from the group consisting of SEQ ID NO:2n, wherein n is an integer between 1 and 127.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 127.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide,
thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 127 or a biologically active fragment thereof.

20. An isolated nucleic acid molecule comprising a nucleic acid sequence selected from the group consisting of SEQ ID NO:2n−1, wherein n is an integer between 1 and 127.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 127.

23. An isolated nucleic acid molecule encoding the mature form of a polypeptide having an amino acid sequence selected from the group consisting of SEQ ID NO:2n, wherein n is an integer between 1 and 127.

24. An isolated nucleic acid molecule comprising a nucleic acid selected from the group consisting of 2n−1, wherein n is an integer between 1 and 127.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 127, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 127.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 127.

42. The method of claim 41 wherein the cell is a bacterial cell.

43. The method of claim 41 wherein the cell is an insect cell.

44. The method of claim 41 wherein the cell is a y east cell.

45. The method of claim 41 wherein the cell is a mammalian cell.
